(19) United States
(12) Patent Application Publication
(10) Pub. No.: US 2023/0115871 A1
(43) Pub. Date: Apr. 13, 2023

(54) A CONJUGATE OF A CYTOTOXIC AGENT TO A CELL BINDING MOLECULE WITH BRANCHED LINKERS

(71) Applicant: Hangzhou DAC Biotech Co., Ltd, Hangzhou, Zhejiang (CN)

(72) Inventors: Robert Yongxin ZHAO, Lexington, MA (US); Qingliang YANG, Hangzhou, Zhejiang (CN); Linyao ZHAO, Hangzhou, Zhejiang (CN); Yuanyuan HUANG, Hangzhou, Zhejiang (CN); Hangbo YE, Hangzhou, Zhejiang (CN); Shun GAI, Hangzhou, Zhejiang (CN); Junxiang JIA, Hangzhou, Zhejiang (CN); Lu BAI, Hangzhou, Zhejiang (CN); Wenjun LI, Hangzhou, Zhejiang (CN); Zhixiang GUO, Hangzhou, Zhejiang (CN); Chen LIN, Hangzhou, Zhejiang (CN); Jun ZHENG, Hangzhou, Zhejiang (CN); Huihui GUO, Hangzhou, Zhejiang (CN); Minjun CAO, Hangzhou, Zhejiang (CN); Xiangfei KONG, Hangzhou, Zhejiang (CN); Yong DU, Hangzhou, Zhejiang (CN); Yifang XU, Hangzhou, Zhejiang (CN); Xiaomai ZHOU, Hangzhou, Zhejiang (CN); Hongsheng XIE, Hangzhou, Zhejiang (CN); Xiuzhen ZHANG, Hangzhou, Zhejiang (CN); Miaomiao CHEN, Hangzhou, Zhejiang (CN); Xiaolei LIU, Hangzhou, Zhejiang (CN); Xiang CAI, Hangzhou, Zhejiang (CN); Binbin CHEN, Hangzhou, Zhejiang (CN); Yanlei YANG, Hangzhou, Zhejiang (CN); Lingli ZHANG, Hangzhou, Zhejiang (CN)

(73) Assignee: Hangzhou DAC Biotech Co., Ltd, Hangzhou, Zhejiang (CN)

(21) Appl. No.: 17/622,360

(22) PCT Filed: Jun. 24, 2019
(86) PCT No.: PCT/CN2019/092614

§ 371 Date: Dec. 23, 2021

(51) Int. Cl. A61K 47/68

(52) U.S. Cl. CPC A61K 47/6889; A61K 47/6803; A61K 47/6835

(57) ABSTRACT

The present invention relates to the conjugation of cytotoxic drug to a cell-binding molecule with a side-chain linker. It provides side-chain linkage methods of making a conjugate of a cytotoxic molecule to a cell-binding ligand, as well as methods of using the conjugate in targeted treatment of cancer, infection and immunological disorders.

### FIELD OF THE INVENTION

The present invention relates to the conjugation of a cytotoxic agent to a cell-binding molecule with branch linkers for having better pharmacokinetics in delivery of the conjugate compound, resulting in much precise targeted treatment of abnormal cells. It also relates to a branch-linkage method of conjugation of a cytotoxic agent to a cell-binding ligand, as well as methods of using the conjugate in targeted prophylaxis or treatment of cancer, infection and immunological disorders.

### BACKGROUND OF THE INVENTION

An antibody-drug conjugate (ADC) which consists of a monoclonal antibody (mAb) linked to a cytotoxic drug via a specialized linking molecule is becoming one of the major biotherapeutics for the treatment of cancers, infection, autoimmune disorder and the other drug resistant diseases (Lambert, J. M. and Berkenblit, A., Annu Rev Med. 2018, 69:191-207; Mariathasan, S. and Tan, M., Trends Mol Med. 2017, 23(2):135-149; Kern, J. C. et al. J. Am. Chem. Soc. 2016, 138, 1430-1445; Lee, H. et al, Bioconjug Chem. 2017, 28(4): 1084-1092). Because the antibody containing 100s-time larger in size structure have in general much longer half-life in blood circulation than that of the cytotoxic drug. Thus, the likelihood of systemic exposure and toxicity of the regular cytotoxic drug once linked to an antibody is greatly reduced. Moreover, ADC is able to pose more precisely a feature of delivering and releasing cytotoxic agents at the tumor site or within the target tumor cells. Therefore, the therapeutic window which is tumor to-normal tissue selectivity and specificity for the cytotoxic drug is much improved. Currently, there are five ADCs approved by the US FDA: gemtuzumab ozogamicin, brentuximab vedotin, trastuzumab emtansine, inotuzumab ozogamicin, and moxetumomab pasudotox, and over 100 new promising agents are under development.

Among the ADC complexes of a cytotoxic drug, a releasable linker, an antibody, and a technology to conjugate the components at the antibody site, it has been known that the linker significantly affects the potency, selectivity and the pharmacokinetics of the resulting ADC conjugate, as well as it can overcome multi-drug resistant ailment cells that overexpress effluxing transporter proteins (Zhao, R. Y. et al (2011) J. Med. Chem. 54, 3606; Acchionea, M. et al (2012) mAbs, 4, 362; Doronina, S. et al, (2006) Bioconjug Chem, 17, 114; Hamann, P. et al. (2005) Bioconjug Chem. 16, 346). Thus, the optimizing linker is of crucial importance for improving the spectrum of the therapeutic potential and safety profiles of ADCs.

Because the linker for ADCs has to be degradable, the conjugated cytotoxic drugs could be potentially released in the blood circulation stream, and therefore increase systemic toxicity and decrease effectiveness. This type off-target toxicity, plus poor permeability/internalization, metabolic liabilities, and target specificity on tumor cells cause over 40 ADC drugs failed in the clinic trials during the past 4 decades. The off-target toxicity also hurdles the expansive application of the approved ADC drugs. For instance, in clinical practice Ado-trastuzumab emtansine (T-DM1, Kadcyla®) which is used stable (none-cleavable) MCC linker has shown great benefit to patients who have HER2-positive metastatic breast cancer (mBC) or who have already been treated for mBC or developed HER2 tumor recurrence within six months of adjuvant therapy (Peddi, P. and Hurvitz, S., Ther. Adv. Med. Oncol. 2014, 6(5), 202-209; Piwko C, et al, Clin Drug Investig. 2015, 35(8), 487-93; Lambert, J. and Chari, R., J. Med. Chem. 2014, 57, 6949-64). But, T-DM1 had failed in clinic trial as first-line treatment for patients with HER2 positive unresectable locally advanced or metastatic breast cancer and as the second line treatment of HER2-positive advanced gastric cancer due to a little benefit to patients when comparison the side toxicity to the efficacy (Ellis, P. A., et al, J. Clin. Oncol. 2015, 33, (suppl; abstr 507 of 2015 ASCO Annual Meeting); Shen, K. et al, Sci Rep. 2016; 6: 23262; de Goeij, B. E. and Lambert, J. M. Curr Opin Immunol 2016, 40, 14-23; Barrios, C. H. et al, J Clin Oncol 2016, 34, (suppl; abstr 593 of 2016 ASCO Annual Meeting).

To address issues of the off-target toxicity, research and development into ADC chemistry and design are now expanding the scopes of the linker-payload compartments and conjugate chemistry beyond the sole potent payloads, and especially to address activity of the linker-payload of ADCs toward targets/target diseases (Lambert, J. M. Ther Deliv 2016, 7, 279-82; Zhao, R. Y. et al, 2011, J. Med. Chem. 54, 3606-23). Nowadays many drug developers and academic institutions are highly focusing on establishing novel reliable specific conjugation linkers and methods for site-specific ADC conjugation, which seem to have longer circulation half-life, higher efficacy, potentially decreased off-target toxicity, and a narrow range of in vivo pharmacokinetic (PK) properties of ADCs as well as better batch-to-batch consistency in ADC production (Hamblett, K. J. et al, Clin. Cancer Res. 2004, 10, 7063-70; Adem, Y. T. et al, Bioconjugate Chem. 2014, 25, 656-664; Boylan, N. J. Bioconjugate Chem. 2013, 24, 1008-1016; Strop, P., et al 2013 Chem. Biol. 20, 161-67; Wakankar, A. mAbs, 2011, 3, 161-172). These specific conjugation methods reported so far include incorporation of engineered cysteines (Junutula, J. R. et al. Nat. Biotechnol. 2008, 26, 925-32; Junutula, J. R., et al 2010 Clin. Cancer Res. 16, 4769; U.S. Pat. Nos. 8,309,300; 7,855,275; 7,521,541; 7,723,485, WO2008/141044), selenocysteines (Hofer, T., et al. Biochemistry 2009, 48, 12047-57; Li, X., et al. Methods 2014, 65, 133-8; U.S. Pat. No. 8,916,159 for US National Cancer Institute), cysteine containing tag with perfluoroaromatic reagents (Zhang, C. et al. Nat. Chem. 2015, 8, 1-9), thiolfucose (Okeley, N. M., et al 2013 Bioconjugate Chem. 24, 1650), non-natural amino acids (Axup, J. Y., et al, Proc. Nat. Acad. Sci. USA. 2012, 109, 16101-6; Zimmerman, E. S., et al., 2014, Bioconjug. Chem. 25, 351-361; Wu, P., et al, 2009 Proc. Natl. Acad. Sci. 106, 3000-5; Rabuka, D., et al, Nat. Protoc. 2012, 7, 1052-67; U.S. Pat. No. 8,778,631 and US Pat Appl. 20100184135, WO2010/081110 for Sutro Biopharma; WO2006/069246, 2007/059312, U.S. Pat. Nos. 7,332,571, 7,696,312, and 7,638,299 for Ambrx; WO2007/130453, U.S. Pat. Nos. 7,632,492 and 7,829,659 for Allozyne), conjugation to reduced intermolecular disulfides by re-bridging dibromomalemides (Jones, M. W. et al. J. Am. Chem. Soc. 2012, 134, 1847-52), bis-sulfone reagents (Badescu, G. et al. Bioconjug. Chem. 2014, 25, 1124-36; WO2013/190272, WO2014/064424 for PolyTherics Ltd). dibromopyridazinediones (Maruani, A. et al. Nat. Commun. 2015, 6, 6645), galactosyl- and sialyltransferases (Zhou, Q. et al. Bioconjug. Chem. 2014, 25, 510-520; US Pat Appl 20140294867 for Sanofi-Genzyme), formylglycine generating enzyme (FGE) (Drake, P. M. et al. Bioconj. Chem. 2014, 25, 1331-41; Carrico, I. S. et al U.S. Pat. Nos. 7,985,783; 8,097,701; 8,349,910, and US Pat Appl 20140141025, 20100210543 for Redwood Bioscience), phosphopantetheinyl transferases (PPTases) (Grunewald, J. et al. Bioconjug. Chem. 2015, 26, 2554-62), sortase A (Beerli, R. R., et al. PLoS One 2015, 10, e0131177), genetically introduced glutamine tag with Streptoverticillium mobaraense transglutaminase (mTG) (Strop, P., Bioconj. Chem., 2014, 25, 855-62; Strop, P., et al., Chem. Biol. 2013, 20, 161-7; U.S. Pat. No. 8,871,908 for Rinat-Pfizer) or with microbial transglutaminase (MTGase) (Dennler, P., et al, 2014, Bioconjug. Chem. 25, 569-78; Siegmund, V. et al. Angew. Chemie—Int. Ed. 2015, 54, 13420-4; US pat appl 20130189287 for Innate Pharma; U.S. Pat. No. 7,893,019 for Bio-Ker S.r.l. (IT)), an enzyme/bacterium forming an isopeptide bond-peptide bonds that form outside of the protein main chain (Kang, H. J., et al. Science 2007, 318, 1625-8; Zakeri, B. et al. Proc. Natl. Acad. Sci. USA 2012, 109, E690-7; Zakeri, B. & Howarth, M. J. Am. Chem. Soc. 2010, 132, 4526-7).

We have disclosed several conjugation methods of rebridging a pair of thiols of the reduced inter chain disulfide bonds of a native antibody, such as using bromo maleimide and dibromomaleimide linkers (WO2014/009774), 2,3-disubstituted succinic/2-monosubstituted/2,3-disubstituted fumaric or maleic linkers (WO2015/155753, WO20160596228), acetylenedicarboxylic linkers (WO2015/151080, WO20160596228) or hydrazine linkers (WO2015/151081). The ADCs made with these linkers and methods have demonstrated better therapeutic index windows than the traditionally unselective conjugation via the cysteine or lysine residues on an antibody. Here we disclose the invention of conjugates of a cytotoxic drug containing a long side chain linker. The long side chain linker can prevent an antibody-drug conjugate from hydrolysis by a hydrolase, e.g. a proteinase or an esterase, and make the conjugate more stable during the targeted delivery and minimize exposure to non-target cells, tissues or organs in the circulation, resulting in longer half-life in the blood stream, less the off-target toxicity d and wider therapeutic windows of the conjugate.

### SUMMARY OF THE INVENTION

The present invention provides branched-linkage of a cytotoxic agent to an antibody. It also provides a method of conjugation of a cytotoxic agent analog to an antibody with the side chain-linker.

In one aspect of the present invention, a conjugate containing a side chain-linkage is represented by Formula (I):

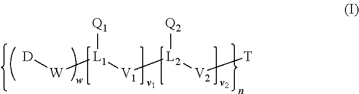

wherein

“ [symbol] ” represents a single bond; n is 1 to 30;

T is a cell-binding agent/molecule, selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and a binding peptide, protein, small molecule attached on albumin, a polymer, a dendrimer, a liposome, a nanoparticle, a vesicle, or a (viral) capsid;

L₁ and L₂ are a chain of atoms selected from C, N, O, S, Si, and P, preferably having 0˜500 atoms, which covalently connects to W and V₁, and V₁ and V₂. The atoms used in forming the L₁ and L₂ may be combined in all chemically relevant ways, such as forming alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof. Preferably L₁ and L₂ are, the same or different, independently selected from O, NH, N, S, P, NNH, NHNH, N(R₃), N(R₃)N(R_{3′}), CH, CO, C(O)NH, C(O)O, NHC(O)NH, NHC(O)O, polyethyleneoxy unit of formula (OCH₂CH₂)ₚOR₃, or (OCH₂CH—(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 1000, or combination thereof; C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or (Aa)ᵣ, r=1-12 (one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;

W is a stretcher unit, normally a self-immolative spacer, a peptidyl unit, a hydrazone, a disulfide, a thioether, an ester, or an amide bond; w is 1 or 2 or 3;

V₁ and V₂ are independently a spacer unit and selected from O, NH, S, C₁-C₈ alkyl, C₂-C₈ heteroalkyl, alkenyl, or alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12 (one to 12 amino acid units), which is composed from a natural or unnatural amino acid, or the same or different sequences of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000; and v₁ and v₂ are independently 0, 1 or 2, but v₁ and v₂ are not 0 at the same time; when v₁ or v₂ is 0, it means that one of the side chain Q₁ or Q₂ fragment is absent.

Q₁ and Q₂ are independently represented by Formula (I-q1):

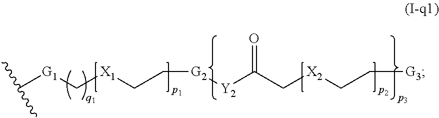

wherein [symbol] is the connecting site linked to L₁ or L₂; G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, (Aa)ᵣ, (r=1-12); X₁ and X₂ are independently O, CH₂, S, NH, N(R₁₂), ⁺NH(R₁₂), ⁺N(R₁₂)(R₁₃), C(O), OC(O), OC(O)O, NHSO₂NH, NHP(O)(NH)₂, SO₂NH, P(O)(NH)₂, NHS(O)NH, NHP(O)(OH)(NH), OC(O)NH, NHC(O)NH; Y₂ is O. NH, NR₁, CH₂. S. Ar; G₃ is OH, SH, OR₁, SR₁, OC(O)R₁, NHC(O)R₁₂, C(O)R₁₂, CH₃, NH₂, NR₁₂, ⁺NH(R₁₂), ⁺N(R₁₂)(R₁₃), C(O)OH, C(O)NH₂, NHC(O)NH₂, BH₂, BR₁₂R₁₃, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁, Ar, ArR₁₂, ArOH, ArNH₂, ArSH, ArNHR₁₂, or (Aa)_{q1}; p₁, p₂ and p₃ are independently 0-100 but are not 0 at the same time; q₁ and q₂ are independently 0-24; Preferably Q₁ and Q₂ are independently, lineal or branched, a C₂-C₉₀ polycarboxylacid or a C₂-C₉₀ polyalkylamine, a C₆-C₉₀ oligosaachride or polysaccharide, a C₆-C₉₀ zwitterionic betaines or zwitterionic poly(sulfobetaine)) (PSB)s that consist of a quarternary ammonium cation and a sulfonate anion, biodegradable polymer (such as composed of poly (lactic/glycolic) acid (PLGA), poly(acrylates), chitosans, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), Poly(ethylene glycol)(PEG), poly(propylene glycol)(PPG), poly(lactide-co-glycolide), poly(ethylene glycol)-modified peptides, poly(ethylene glycol)-modified lipids, poly(ethylene glycol)-modified alkylcarboxic acid, poly(ethylene glycol)-modified alkylamine, poly(lactide)-co-glycolide, polysarcosine, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quarternary ammonium; D is a cytotoxic agent that is independently selected from calicheamicins, camptothecins, maytansinoids, taxanes, daunorubicin/doxorubicin, vinca alkaloids, auristatins, eribulins, pyrrolobenzodiazepines (PBDs), duocarmycins, kinase inhibitors, MEK inhibitors, KSP inhibitors, nicotinamide phosphoribosyltransferase (NAMPT) inhibitors, immunotoxins, analogs or prodrugs thereof.

In another aspect of the present invention, a conjugate containing a side chain-linkage is represented by Formula (II), and (III):

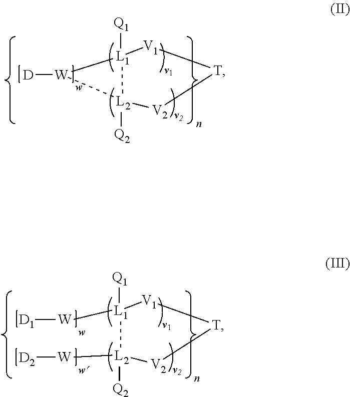

wherein D, W, L₁, L₂, Q₁, Q₂, V₁, V₂, v₁, v₂, n, T are defined the same as in Formula (I); w and w′ are independently 1, 2 or 3; and - - - - - is a single bond, double bond or absent; D₁ and D₂ are the same or different, and they are defined the same as D.

In another aspect of the present invention, the side chain-linkage compound is represented by Formula (IV), which can readily react to a cell-binding molecule T to form a conjugate of Formula (I):

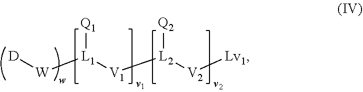

wherein D, W, w, L₁, L₂, Q₁, Q₂, V₁, V₂, v₁, v₂, and n, are defined the same as in Formula (I); Lv1 is a function group described below.

In another aspect of the present invention, the side chain-linkage compound is represented by Formula (V), and (VI) which can readily react to a pair of sites on a cell-binding molecule T to form a conjugate of Formula (II), and (III) respectively:

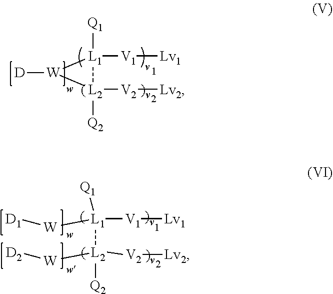

wherein D, D₁, D₂, W, w, w′, L₁, L₂, Q₁, Q₂, V₁, V₂, v₁, v₂, - - - - - - and n, are defined the same as above.

Lv₁ and Lv₂ represent the same or different reacting group that can be reacted with a thiol, amine, carboxylic acid, selenol, phenol or hydroxyl group on a cell-binding molecule.

Lv₁ and Lv₂ are independently selected from OH; F; Cl; Br; I; nitrophenol; N-hydroxysuccinimide (NHS); phenol; dinitrophenol; pentafluorophenol; tetrafluorophenol; difluorophenol; mono-fluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions, or for Mitsunobu reactions. The examples of condensation reagents are: EDC (N-(3-Dimethylaminopropyl)-N′-ethylcarbodiimide), DCC (Dicyclohexyl-carbodiimide), N,N′-Diisopropylcarbodiimide (DIC), N-Cyclohexyl-N′-(2-morpholino-ethyl)carbodiimide metho-p-toluenesulfonate (CMC, or CME-CDI), 1,1′-Carbonyldiimidazole (CDI), TBTU (O-(Benzotriazol-1-yl)-N,N,N′,N′-tetramethyluronium tetrafluoroborate), N,N,N′,N′-Tetramethyl-O-(1H-benzotriazol-1-yl)-uronium hexafluoro-phosphate (HBTU), (Benzotriazol-1-yloxy)tris(dimethylamino)-phosphonium hexafluorophosphate (BOP), (Benzotriazol-1-yloxy)tripyrrolidinophosphonium hexafluorophosphate (PyBOP), Diethyl cyanophosphonate (DEPC), Chloro-N,N,N′,N′-tetramethylformamidiniumhexafluorophosphate, 1-[Bis(dimethylamino)methylene]-1H-1,2,3-triazolo[4,5-b]pyridinium 3-oxid hexafluorophosphate (HATU), 1-[(Dimethylamino)(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetramethylene)formamidinium hexafluorophosphate (BTFFH), N,N,N′,N′-Tetramethyl-S-(1-oxido-2-pyridyl)thiuronium hexafluorophosphate, O-(2-Oxo-1(2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate (HOTU), (1-Cyano-2-ethoxy-2-oxoethylidenaminooxy) dimethylamino-morpholino-carbenium hexafluorophosphate (COMU), O-(Benzotriazol-1-yl)-N,N,N′,N′-bis(tetramethylene)uronium hexafluorophosphate (HBPyU), N-Benzyl-N′-cyclohexyl-carbodiimide (with, or without polymer-bound), Dipyrrolidino(N-succinimidyl-oxy)carbenium hexafluoro-phosphate (HSPyU), Chlorodipyrrolidinocarbenium hexafluorophosphate (PyClU), 2-Chloro-1,3-dimethylimidazolidinium tetrafluoroborate(CIB), (Benzotriazol-1-yloxy)dipiperidino-carbenium hexafluorophosphate (HBPipU), O-(6-Chlorobenzotriazol-1-yl)-N,N,N′,N′-tetramethyluronium tetrafluoroborate (TCTU), Bromotris(dimethylamino)-phosphonium hexafluorophosphate (BroP), Propylphosphonic anhydride (PPACA, T3P©), 2-Morpholinoethyl isocyanide (MEI), N,N,N′,N′-Tetramethyl-O—(N-succinimidyl)uronium hexafluorophosphate (HSTU), 2-Bromo-1-ethyl-pyridinium tetrafluoroborate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholiniumchloride (MMTM, DMTMM), N,N,N′,N′-Tetramethyl-O—(N-succinimidyl)uronium tetrafluoroborate (TSTU), O-(3,4-Dihydro-4-oxo-1,2,3-benzotriazin-3-yl)-N,N,N′,N′-tetramethyluronium tetrafluoro-borate (TDBTU), 1,1′-(Azodicarbonyl)-dipiperidine (ADD), Di-(4-chlorobenzyl)azodicarboxylate (DCAD), Di-tert-butyl azodicarboxylate (DBAD), Diisopropyl azodicarboxylate (DIAD), Diethyl azodicarboxylate (DEAD). In addition, Lv₁ and Lv₂ can be an anhydride, formed by acid themselves or formed with other C₁˜C₈ acid anhydrides; The present invention further relates to a method of making a cell-binding molecule-drug conjugate of Formula (I) and Formula (II) as well the application of the conjugates of Formula (I) and Formula (II).

### BRIEF DESCRIPTION OF THE DRAWINGS

FIG. 1 shows the synthesis of a component of a tubulysin analog containing a linker.

FIG. 2 shows the synthesis of a component of a linear linker.

FIG. 3 shows the synthesis of a tubulysin analog having side-chain linkers.

FIG. 4 shows the synthesis of a tubulysin analog having side-chain linkers.

FIG. 5 shows the synthesis of a fragment of tubulysin analog containing a side-chain linker.

FIG. 6 shows the synthesis of conjugates of tubulysin analogs containing side-chain linkers.

FIG. 7 shows the syntheses of exatecan and fragments of a side-chain linker.

FIG. 8 shows the synthesis of conjugates of exatecan containing side-chain linkers.

FIG. 9 shows the synthesis of components of a linear linker.

FIG. 10 shows the general synthesis of drug-linker components containing side-chain linkers.

FIG. 11 shows the synthesis of a conjugate of drug containing a side-chain linker and a maytansinoid-linker component having a side-chain linker.

FIG. 12 shows the synthesis of conjugates of a maytansinoid and an exatecan containing side-chain linkers.

FIG. 13 shows the synthesis of a conjugate of MMAE analog containing a side-chain linker.

FIG. 14 shows the synthesis of a conjugate of MMAF analog containing a side-chain linker.

FIG. 15 shows the synthesis of conjugatable eribulin containing side-chain linkers.

FIG. 16 shows the syntheses of a conjugate of eribulin containing side-chain linkers and a conjugatable CBI-dimer containing side-chain linkers.

FIG. 17 shows the synthesis of a conjugate of a CBI-dimer containing side-chain linkers and a conjugate of a topotecan analog containing side-chain linkers.

FIG. 18 shows the synthesis of a conjugate of a tubulysin analog containing side-chain linkers and a conjugate of a MMAE analog containing side-chain linkers.

FIG. 19 shows the synthesis of a conjugate of a tubulysin analog containing side-chain linkers.

FIG. 20 shows the comparison of the anti-tumor effect of conjugate compounds 49 (C-30), 51 (C-48), C-173, C-238, C-312, 132 (C-131), 135 (C-134), C-321, and C-322, with T-DM1 using human gastric tumor N87 cell model, i.v., one injection at dosing of 6 mg/kg.

FIG. 21 shows an acute toxicity study on ADC conjugates 49 (C-30), 51 (C-48), C-173, C-238, C-312, 132 (C-131), 135 (C-134), C-321, and C-322, and T-DM1 through observing changes in body weight (BW) of mice in 12 days.

### DETAILED DESCRIPTION OF THE INVENTION

### Definitions

“Alkyl” refers to an aliphatic hydrocarbon group or univalent groups derived from alkane by removal of one or two hydrogen atoms from carbon atoms. It may be straight or branched having C₁-C₈ (1 to 8 carbon atoms) in the chain. “Branched” means that one or more lower C numbers of alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl, cyclopentyl, cyclohexyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methyl-hexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, n-heptyl, isoheptyl, n-octyl, and isooctyl. A C₁-C₈ alkyl group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)₂R′, —S(O)R′, —OH, -halogen, —N₃, —NH₂, —NH(R′), —N(R′)₂ and —CN; where each R′ is independently selected from —C₁-C₈ alkyl and aryl.

“Halogen” refers to fluorine, chlorine, bromine or iodine atom; preferably fluorine and chlorine atom.

“Heteroalkyl” refers to C₂-C₈ alkyl in which one to four carbon atoms are independently replaced with a heteroatom from the group consisting of O, S and N.

“Carbocycle” refers to a saturated or unsaturated ring having 3 to 8 carbon atoms as a monocycle or 7 to 13 carbon atoms as a bicycle. Monocyclic carbocycles have 3 to 6 ring atoms, more typically 5 or 6 ring atoms. Bicyclic carbocycles have 7 to 12 ring atoms, arranged as a bicycle [4,5], [5,5], [5,6] or [6,6] system, or 9 or 10 ring atoms arranged as a bicycle [5,6] or [6,6] system. Representative C₃-C₈ carbocycles include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl.

A “C₃-C₈ carbocycle” refers to a 3-, 4-, 5-, 6-, 7- or 8-membered saturated or unsaturated nonaromatic carbocyclic ring. A C₃-C₈ carbocycle group can be unsubstituted or substituted with one or more groups including, but not limited to, —C₁-C₈ alkyl, —O—(C₁-C₈ alkyl), -aryl, —C(O)R′, —OC(O)R′, —C(O)OR′, —C(O)NH₂, —C(O)NHR′, —C(O)N(R′)₂, —NHC(O)R′, —SR′, —S(O)R′, —S(O)₂R′, —OH, -halogen, —N₃, —NH₂, —NH(R′), —N(R′)₂ and —CN; where each R′ is independently selected from —C₁-C₈ alkyl and aryl.

“Alkenyl” refers to an aliphatic hydrocarbon group containing a carbon-carbon double bond which may be straight or branched having 2 to 8 carbon atoms in the chain. Exemplary alkenyl groups include ethenyl, propenyl, n-butenyl, i-butenyl, 3-methylbut-2-enyl, n-pentenyl, hexylenyl, heptenyl, octenyl.

“Alkynyl” refers to an aliphatic hydrocarbon group containing a carbon-carbon triple bond which may be straight or branched having 2 to 8 carbon atoms in the chain. Exemplary alkynyl groups include ethynyl, propynyl, n-butynyl, 2-butynyl, 3-methylbutynyl, 5-pentynyl, n-pentynyl, hexylynyl, heptynyl, and octynyl.

“Alkylene” refers to a saturated, branched or straight chain or cyclic hydrocarbon radical of 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

“Alkynylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkyne. Typical alkynylene radicals include, but are not limited to: acetylene, propargyl and 4-pentynyl.

“Aryl” or Ar refers to an aromatic or hetero aromatic group, composed of one or several rings, comprising three to fourteen carbon atoms, preferentially six to ten carbon atoms. The term of “hetero aromatic group” refers one or several carbon on aromatic group, preferentially one, two, three or four carbon atoms are replaced by O, N, Si, Se, P or S, preferentially by O, S, and N. The term aryl or Ar also refers to an aromatic group, wherein one or several H atoms are replaced independently by —R′, -halogen, —OR′, or —SR′, —NR′R″, —N═NR′, —N═R′, —NR′R″, —NO₂, —S(O)R′, —S(O)₂R′, —S(O)₂OR′, —OS(O)₂OR′, —PR′R″, —P(O)R′R″, —P(OR′)(OR″), —P(O)(OR′)(OR″) or —OP(O)(OR′)(OR″) wherein R′, R″ are independently H, alkyl, alkenyl, alkynyl, heteroalkyl, aryl, arylalkyl, carbonyl, or pharmaceutical salts.

“Heterocycle” refers to a ring system in which one to four of the ring carbon atoms are independently replaced with a heteroatom from the group of O, N, S, Se, B, Si and P. Preferable heteroatoms are O, N and S. Heterocycles are also described in The Handbook of Chemistry and Physics, 78th Edition, CRC Press, Inc., 1997-1998, p. 225 to 226, the disclosure of which is hereby incorporated by reference. Preferred nonaromatic heterocyclic include epoxy, aziridinyl, thiiranyl, pyrrolidinyl, pyrazolidinyl, imidazolidinyl, oxiranyl, tetrahydrofuranyl, dioxolanyl, tetrahydropyranyl, dioxanyl, dioxolanyl, piperidyl, piperazinyl, morpholinyl, pyranyl, imidazolinyl, pyrrolinyl, pyrazolinyl, thiazolidinyl, tetrahydrothiopyranyl, dithianyl, thiomorpholinyl, dihydropyranyl, tetrahydropyranyl, dihydropyranyl, tetrahydropyridyl, dihydropyridyl, tetrahydropyrimidinyl, dihydrothiopyranyl, azepanyl, as well as the fused systems resulting from the condensation with a phenyl group.

The term “heteroaryl” or aromatic heterocycles refers to a 3 to 14, preferably 5 to 10 membered aromatic hetero, mono-, bi-, or multi-cyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1,2,4-thiadiazolyl, isothiazolyl, triazolyl, tetrazolyl, isoquinolyl, benzothienyl, isobenzofuryl, pyrazolyl, carbazolyl, benzimidazolyl, isoxazolyl, pyridyl-N-oxide, as well as the fused systems resulting from the condensation with a phenyl group.

“Alkyl”, “cycloalkyl”, “alkenyl”, “alkynyl”, “aryl”, “heteroaryl”, “heterocyclic” and the like refer also to the corresponding “alkylene”, “cycloalkylene”, “alkenylene”, “alkynylene”, “arylene”, “heteroarylene”, “heterocyclene” and the likes which are formed by the removal of two hydrogen atoms.

“Arylalkyl” refers to an acyclic alkyl radical in which one of the hydrogen atoms bonded to a carbon atom, typically a terminal or sp³ carbon atom, is replaced with an aryl radical. Typical arylalkyl groups include, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like.

“Heteroarylalkyl” refers to an acyclic alkyl radical in which one of the hydrogen atoms bonded to a carbon atom, typically a terminal or sp³ carbon atom, is replaced with a heteroaryl radical. Examples of heteroarylalkyl groups are 2-benzimidazolylmethyl, 2-furylethyl.

Examples of a “hydroxyl protecting group” includes, methoxymethyl ether, 2-methoxyethoxymethyl ether, tetrahydropyranyl ether, benzyl ether, p-methoxybenzyl ether, trimethylsilyl ether, triethylsilyl ether, triisopropylsilyl ether, t-butyldimethylsilyl ether, triphenylmethylsilyl ether, acetate ester, substituted acetate esters, pivaloate, benzoate, methanesulfonate and p-toluenesulfonate.

“Leaving group” refers to a functional group that can be substituted by another functional group. Such leaving groups are well known in the art, and examples include, a halide (e.g., chloride, bromide, and iodide), methanesulfonyl (mesyl), p-toluenesulfonyl (tosyl), trifluoromethylsulfonyl (triflate), and trifluoromethylsulfonate. A preferred leaving group is selected from nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

The following abbreviations may be used herein and have the indicated definitions: Boc, tert-butoxy carbonyl; BroP, bromotrispyrrolidinophosphonium hexafluorophosphate; CDI, 1,1′-carbonyldiimidazole; DCC, dicyclohexylcarbodiimide; DCE, dichloroethane; DCM, dichloromethane; DEAD is diethylazodicarboxylate, DIAD, diisopropylazodicarboxylate; DIBAL-H, diisobutyl-aluminium hydride; DIPEA or DEA, diisopropylethylamine; DEPC, diethyl phosphorocyanidate; DMA, N,N-dimethyl acetamide; DMAP, 4-(N, N-dimethylamino)pyridine; DMF, N,N-dimethylformamide; DMSO, dimethylsulfoxide; DTPA is diethylenetriaminepentaacetic acid; DTT, dithiothreitol; EDC, 1-(3-dimethylaminopropyl)-3-ethylcarbodiimide hydrochloride; ESI-MS, electrospray mass spectrometry; EtOAc is ethyl acetate; Fmoc is N-(9-fluorenylmethoxycarbonyl); HATU, O-(7-azabenzotriazol-1-yl)-N, N, N′, N′-tetramethyluronium hexafluorophosphate; HOBt, 1-hydroxybenzotriazole; HPLC, high pressure liquid chromatography; NHS, N-Hydroxysuccinimide; MeCN is acetonitrile; MeOH is methanol; MMP, 4-methylmorpholine; PAB, p-aminobenzyl; PBS, phosphate-buffered saline (pH 7.0˜7.5); Ph is phenyl; phe is L-phenylalanine; PyBrop is bromo-tris-pyrrolidino-phosphonium hexafluorophosphate; PEG, polyethylene glycol; SEC, size-exclusion chromatography; TCEP, tris(2-carboxyethyl)phosphine; TFA, trifluoroacetic acid; THF, tetrahydrofuran; Val, valine; TLC is thin layer chromatography; UV is ultraviolet.

The “amino acid(s)” can be natural and/or unnatural amino acids, preferably alpha-amino acids. Natural amino acids are those encoded by the genetic code, which are alanine, arginine, asparagine, aspartic acid, cysteine, glutamic acid, glutamine, glycine, histidine, isoleucine, leucine, lysine, methionine, phenylalanine, proline, serine, threonine, tyrosine. tryptophan and valine. The unnatural amino acids are derived forms of proteinogenic amino acids. Examples include hydroxyproline, lanthionine, 2-aminoisobutyric acid, dehydroalanine, gamma-aminobutyric acid (the neurotransmitter), ornithine, citrulline, beta alanine (3-aminopropanoic acid), gamma-carboxyglutamate, selenocysteine (present in many noneukaryotes as well as most eukaryotes, but not coded directly by DNA), pyrrolysine (found only in some archaea and one bacterium), N-formylmethionine (which is often the initial amino acid of proteins in bacteria, mitochondria, and chloroplasts), 5-hydroxytryptophan, L-dihydroxyphenylalanine, triiodothyronine, L-3,4-dihydroxyphenylalanine (DOPA), and O-phosphoserine. The term amino acid also includes amino acid analogs and mimetics. Analogs are compounds having the same general H₂N(R)CHCO₂H structure of a natural amino acid, except that the R group is not one found among the natural amino acids. Examples of analogs include homoserine, norleucine, methionine-sulfoxide, and methionine methyl sulfonium. Preferably, an amino acid mimetic is a compound that has a structure different from the general chemical structure of an alpha-amino acid but functions in a manner similar to one. The term “unnatural amino acid” is intended to represent the “D” stereochemical form, the natural amino acids being of the “L” form. When 1-8 amino acids are used in this patent application, amino acid sequence is then preferably a cleavage recognition sequence for a protease. Many cleavage recognition sequences are known in the art. See, e.g., Matayoshi et al. Science 247: 954 (1990); Dunn et al. Meth. Enzymol. 241: 254 (1994); Seidah et al. Meth. Enzymol. 244: 175 (1994); Thornberry, Meth. Enzymol. 244: 615 (1994); Weber et al. Meth. Enzymol. 244: 595 (1994); Smith et al. Meth. Enzymol. 244: 412 (1994); and Bouvier et al. Meth. Enzymol. 248: 614 (1995); the disclosures of which are incorporated herein by reference. In particular, the sequence is selected from the group consisting of Val-Cit, Ala-Val, Ala-Ala, Val-Val, Val-Ala-Val, Lys-Lys, Ala-Asn-Val, Val-Leu-Lys, Cit-Cit, Val-Lys, Ala-Ala-Asn, Asp-Lys, Asp-Glu, Glu-Lys, Lys, Cit, Ser, and Glu.

The “glycoside” is a molecule in which a sugar group is bonded through its anomeric carbon to another group via a glycosidic bond. Glycosides can be linked by an O- (an O-glycoside), N- (a glycosylamine), S- (a thioglycoside), or C- (a C-glycoside) glycosidic bond. Its core the empirical formula is Cₘ(H₂O)ₙ (where m could be different from n, and m and n are <36), Glycoside herein includes glucose (dextrose), fructose (levulose) allose, altrose, mannose, gulose, iodose, galactose, talose, galactosamine, glucosamine, sialic acid, N-acetylglucosamine, sulfoquinovose (6-deoxy-6-sulfo-D-glucopyranose), ribose, arabinose, xylose, lyxose, sorbitol, mannitol, sucrose, lactose, maltose, trehalose, maltodextrins, raffinose, Glucuronic acid (glucuronide), and stachyose. It can be in D form or L form, 5 atoms cyclic furanose forms, 6 atoms cyclic pyranose forms, or acyclic form, α-isomer (the —OH of the anomeric carbon below the plane of the carbon atoms of Haworth projection), or a β-isomer (the —OH of the anomeric carbon above the plane of Haworth projection). It is used herein as a monosaccharide, disaccharide, polyols, or oligosaccharides containing 3-6 sugar units.

The term “antibody,” as used herein, refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce auto-immune antibodies associated with an autoimmune disease. The immunoglobulin disclosed herein can be of any type (e.g. IgG, IgE, IgM, IgD, IgA and IgY), class (e.g., IgG1, IgG2, IgG3, IgG4, IgA1 and IgA2) or subclass of immunoglobulin molecule. The immunoglobulins can be derived from any species. Preferably, however, the immunoglobulin is of human, murine, or rabbit origin. Antibodies useful in the invention are preferably monoclonal, and include, but are not limited to, polyclonal, monoclonal, bispecific, human, humanized or chimeric antibodies, single chain antibodies, Fv, Fab fragments, F(ab′) fragments, F(ab′)₂ fragments, fragments produced by a Fab expression library, anti-idiotypic (anti-Id) antibodies, CDR's, and epitope-binding fragments of any of the above which immunospecifically bind to cancer cell antigens, viral antigens or microbial antigens.

An “enantiomer”, also known as an “optical isomer”, is one of two stereoisomers that are mirror images of each other that are non-superposable (not identical), much as one's left and right hands are the same except for being reversed along one axis (the hands cannot be made to appear identical simply by reorientation). A single chiral atom or similar structural feature in a compound causes that compound to have two possible structures which are non-superposable, each a mirror image of the other. The presence of multiple chiral features in a given compound increases the number of geometric forms possible, though there may be some perfect-mirror-image pairs. Enantiopure compounds refer to samples having, within the limits of detection, molecules of only one chirality. When present in a symmetric environment, enantiomers have identical chemical and physical properties except for their ability to rotate plane-polarized light (+/−) by equal amounts but in opposite directions (although the polarized light can be considered an asymmetric medium). They are sometimes called optical isomers for this reason. A mixture of equal parts of an optically active isomer and its enantiomer is termed racemic and has zero net rotation of plane-polarized light because the positive rotation of each (+) form is exactly counteracted by the negative rotation of a (−) one. Enantiomer members often have different chemical reactions with other enantiomer substances. Since many biological molecules are enantiomers, there is sometimes a marked difference in the effects of two enantiomers on biological organisms. In drugs, for example, often only one of a drug's enantiomers is responsible for the desired physiologic effects, while the other enantiomer is less active, inactive, or sometimes even productive of adverse effects. Owing to this discovery, drugs composed of only one enantiomer (“enantiopure”) can be developed to enhance the pharmacological efficacy and sometimes eliminate some side effects.

Isotopes are variants of a particular chemical element which differs in neutron number. All isotopes of a given element have the same number of protons in each atom. Each atomic number identifies a specific element, but not the isotope; an atom of a given element may have a wide range in its number of neutrons. The number of nucleons (both protons and neutrons) in the nucleus is the atom's mass number, and each isotope of a given element has a different mass number. For example, carbon-12, carbon-13 and carbon-14 are three isotopes of the element carbon with mass numbers 12, 13 and 14 respectively. The atomic number of carbon is 6, which means that every carbon atom has 6 protons, so that the neutron numbers of these isotopes are 6, 7 and 8 respectively. Hydrogen atom has three isotopes of protium (H), deuterium (²H), and tritium (³H), which deuterium has twice the mass of protium and tritium has three times the mass of protium. Isotopic substitution can be used to determine the mechanism of a chemical reaction and via the kinetic isotope effect. Isotopic substitution can be used to study how the body affects a specific xenobiotic/chemical after administration through the mechanisms of absorption and distribution, as well as the metabolic changes of the substance in the body (e.g. by metabolic enzymes such as cytochrome P450 or glucuronosyltransferase enzymes), and the effects and routes of excretion of the metabolites of the drug. This study is called pharmacokinetics (PK). Isotopic substitution can be used to study of the biochemical and physiologic effects of drugs. The effects can include those manifested within animals (including humans), microorganisms, or combinations of organisms (for example, infection). This study is called pharmacodynamics (PD). The effects can include those manifested within animals (including humans), microorganisms, or combinations of organisms (for example, infection). Both together influence dosing, benefit, and adverse effects of the drug. isotopes can contain a stable (non-radioactive) or an unstable element. Isotopic substitution of a drug may have a different therapeutical efficacy of the original drug.

“Pharmaceutically” or “pharmaceutically acceptable” refer to molecular entities and compositions that do not produce an adverse, allergic or other untoward reaction when administered to an animal, or a human, as appropriate.

“Pharmaceutically acceptable solvate” or “solvate” refer to an association of one or more solvent molecules and a disclosed compound. Examples of solvents that form pharmaceutically acceptable solvates include, but are not limited to, water, isopropanol, ethanol, methanol, DMSO, ethyl acetate, acetic acid and ethanolamine.

“Pharmaceutically acceptable excipient” includes any carriers, diluents, adjuvants, or vehicles, such as preserving or antioxidant agents, fillers, disintegrating agents, wetting agents, emulsifying agents, suspending agents, solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions as suitable therapeutic combinations.

As used herein, “pharmaceutical salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucuronic, glutamic, benzoic, salicylic, toluenesulfonic, oxalic, fumaric, maleic, lactic and the like. Further addition salts include ammonium salts such as tromethamine, meglumine, epolamine, etc., metal salts such as sodium, potassium, calcium, zinc or magnesium.

The pharmaceutical salts of the present invention can be synthesized from the parent compound which contains a basic or acidic moiety by conventional chemical methods. Generally, such salts can be prepared via reaction the free acidic or basic forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, the disclosure of which is hereby incorporated by reference.

“Administering” or “administration” refers to any mode of transferring, delivering, introducing or transporting a pharmaceutical drug or other agent to a subject. Such modes include oral administration, topical contact, intravenous, intraperitoneal, intramuscular, intralesional, intranasal, subcutaneous or intrathecal administration. Also contemplated by the present invention is utilization of a device or instrument in administering an agent. Such device may utilize active or passive transport and may be slow-release or fast-release delivery device.

In the context of cancer, the term “treating” includes any or all of: preventing growth of tumor cells or cancer cells, preventing replication of tumor cells or cancer cells, lessening of overall tumor burden and ameliorating one or more symptoms associated with the disease.

In the context of an autoimmune disease, the term “treating” includes any or all of: preventing replication of cells associated with an autoimmune disease state including, but not limited to, cells capable of producing an autoimmune antibody, lessening the autoimmune-antibody burden and ameliorating one or more symptoms of an autoimmune disease.

In the context of an infectious disease, the term “treating” includes any or all of: preventing the growth, multiplication or replication of the pathogen that causes the infectious disease and ameliorating one or more symptoms of an infectious disease.

Examples of a “mammal” or “animal” include, but are not limited to, a human, rat, mouse, guinea pig, monkey, pig, goat, cow, horse, dog, cat, bird and fowl.

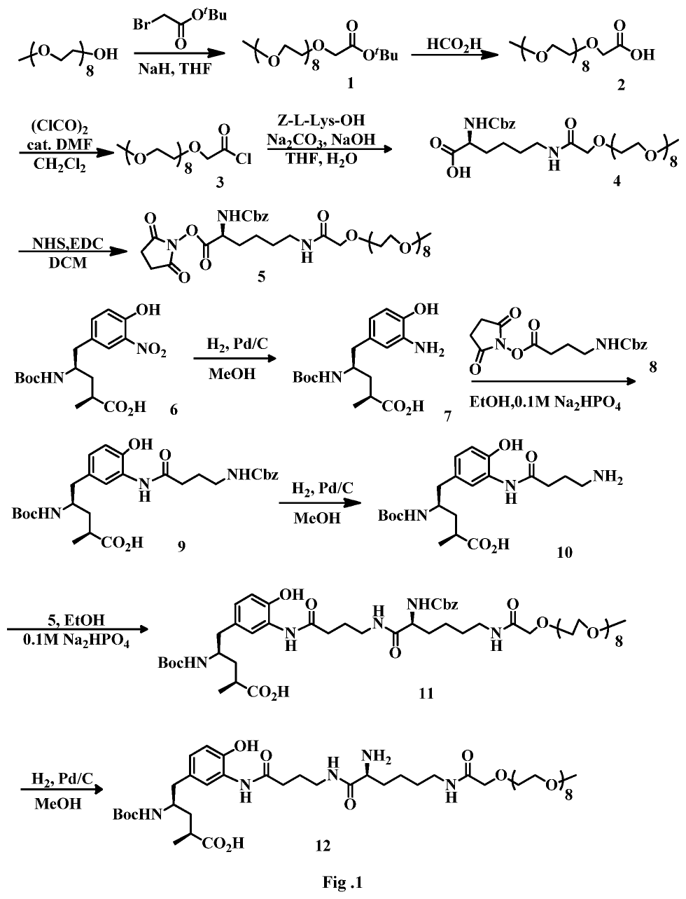

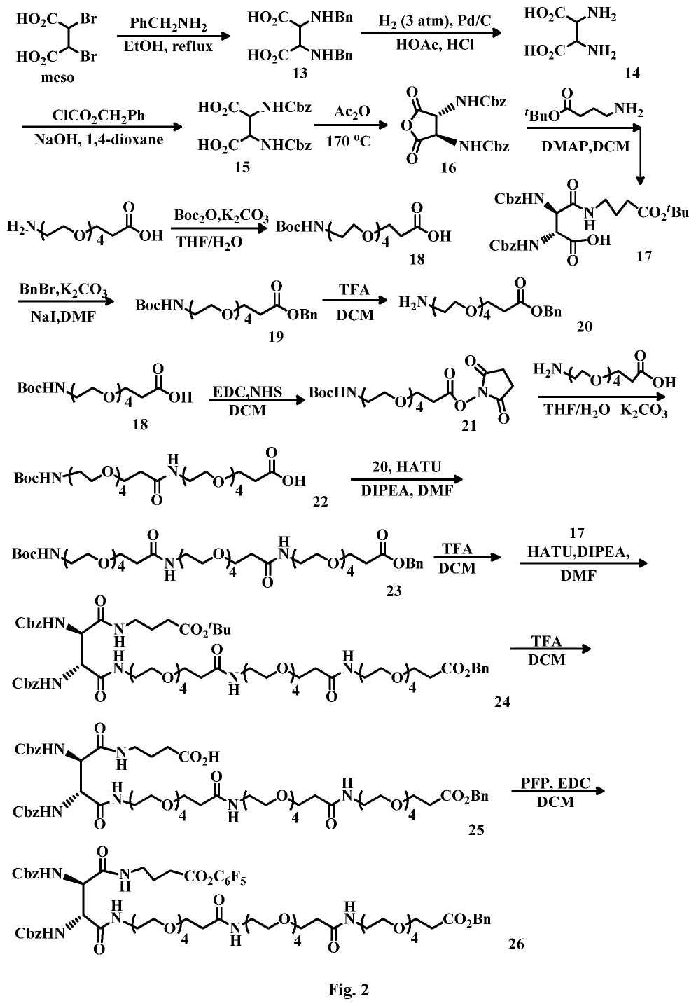

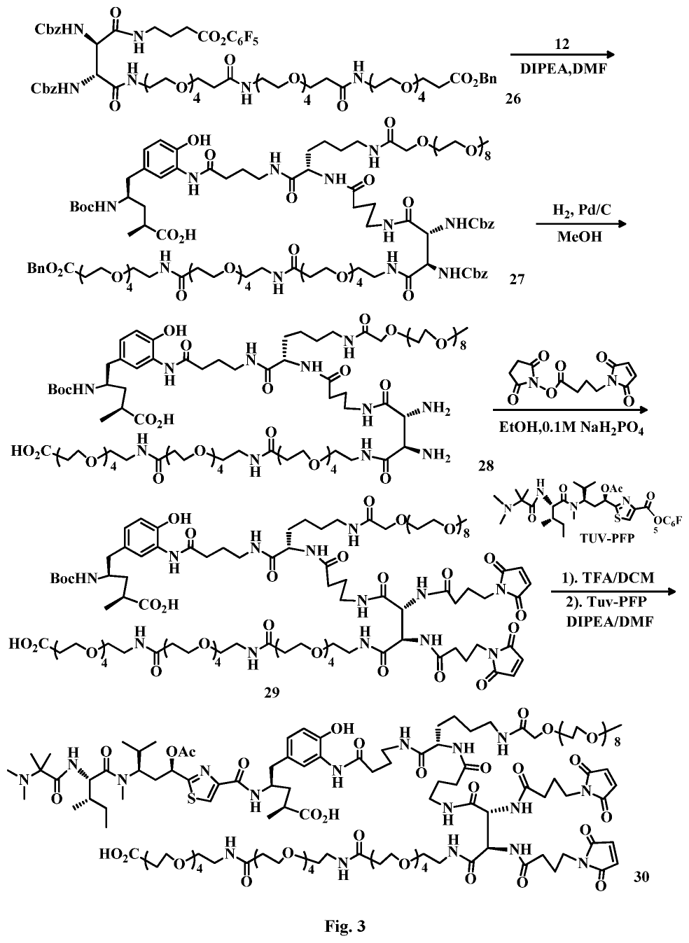

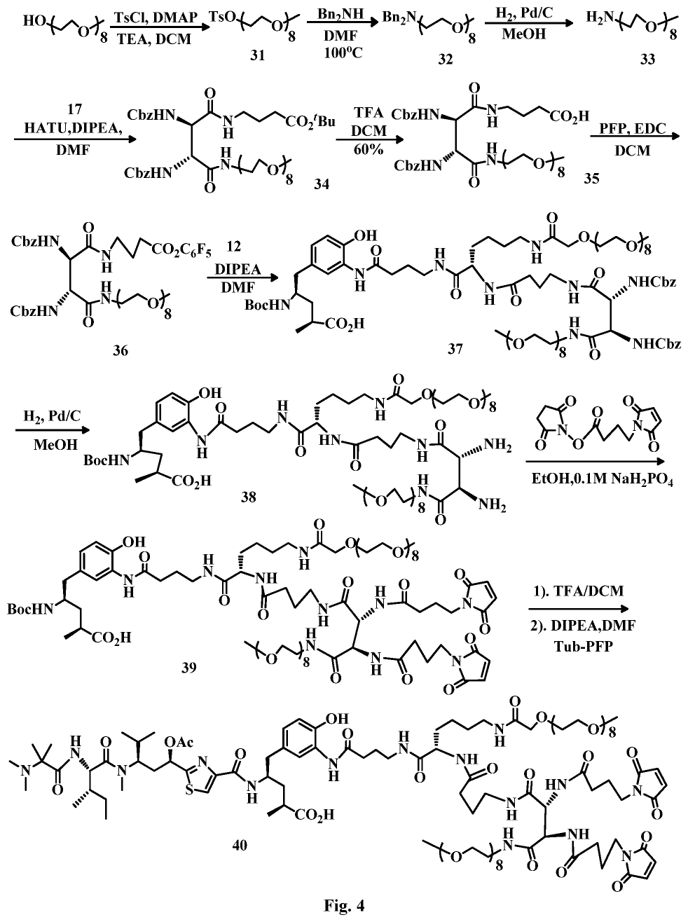

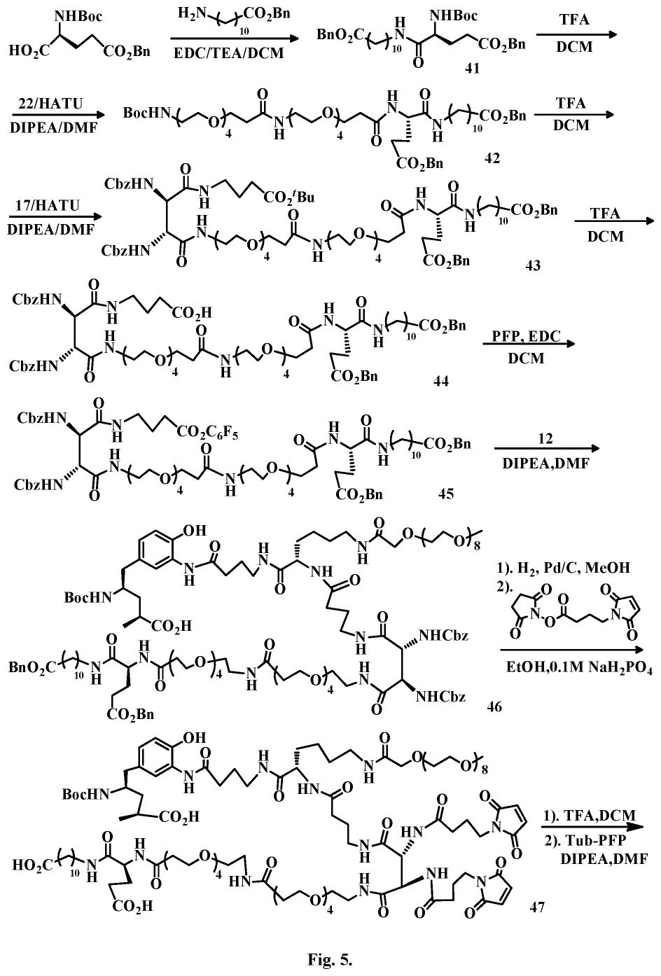

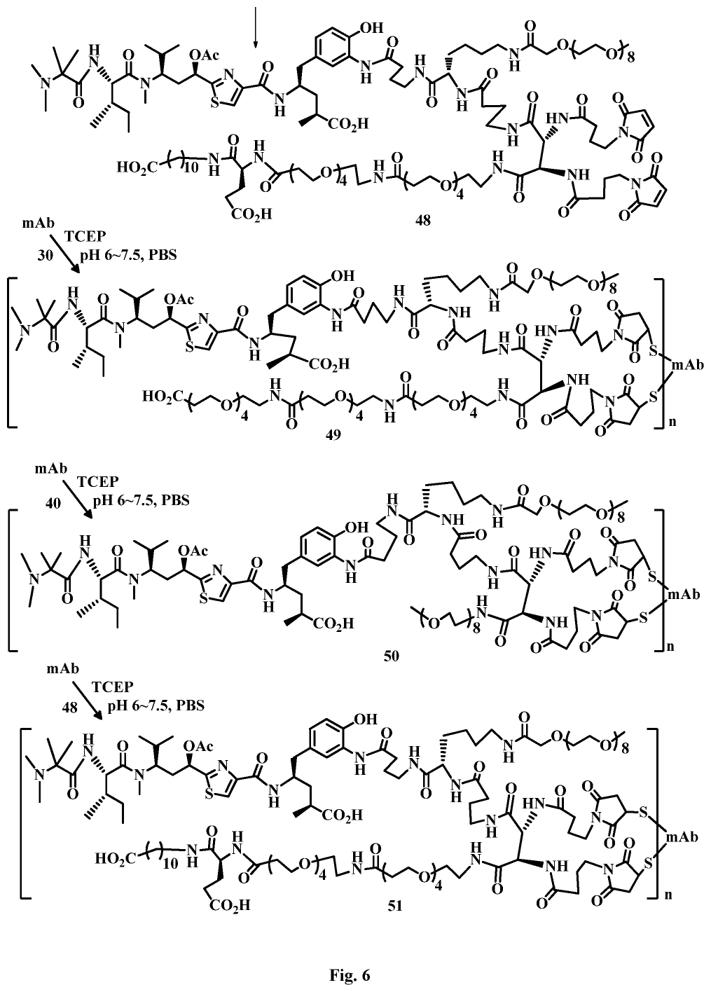

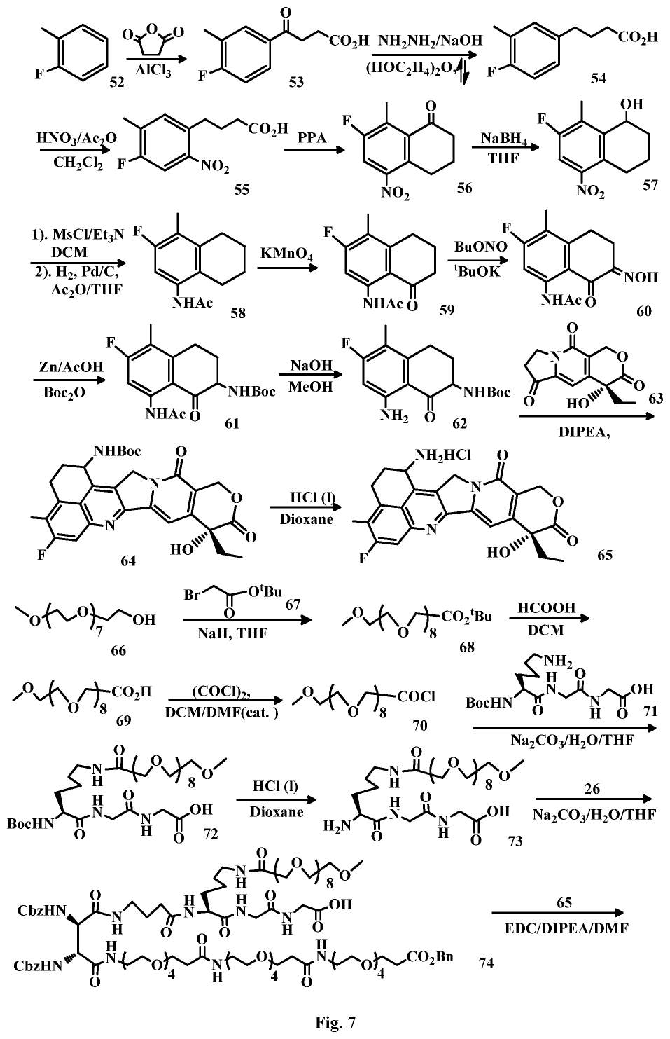

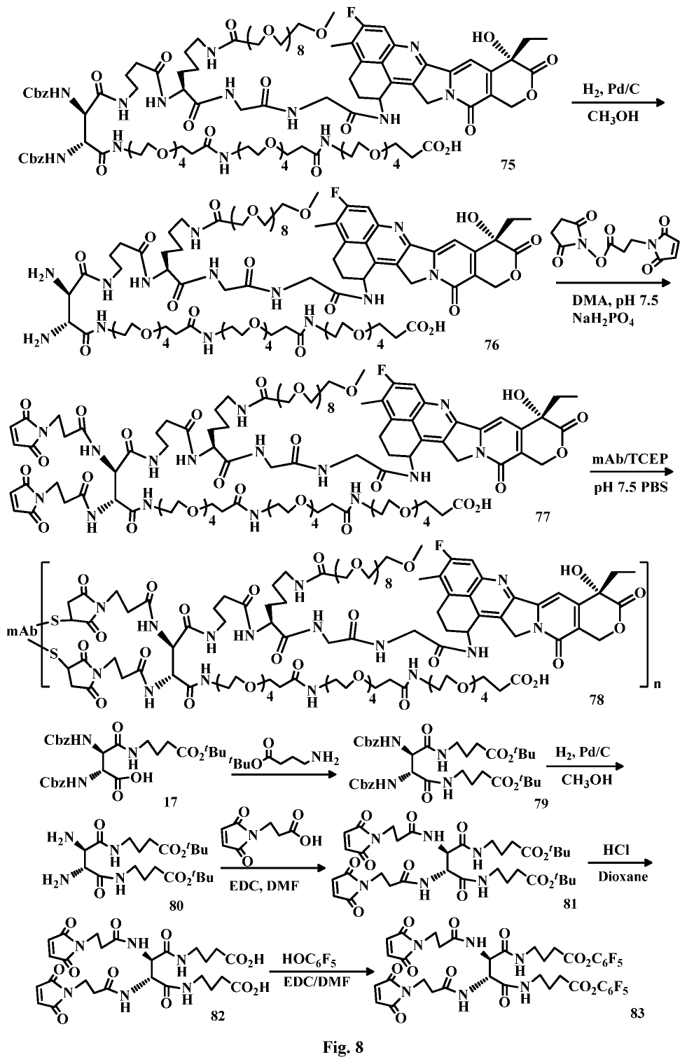

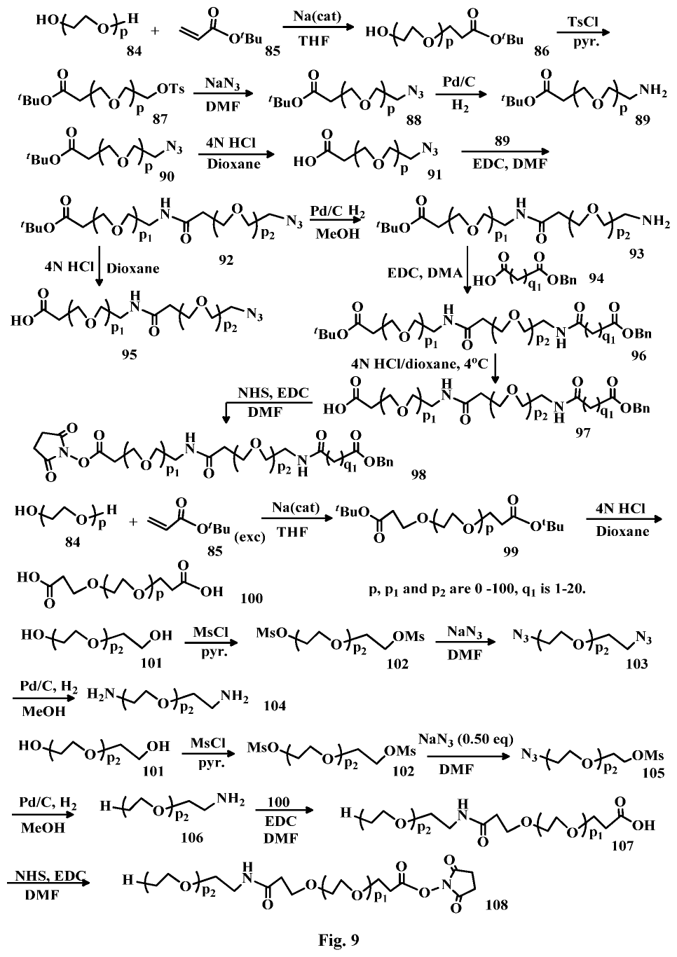

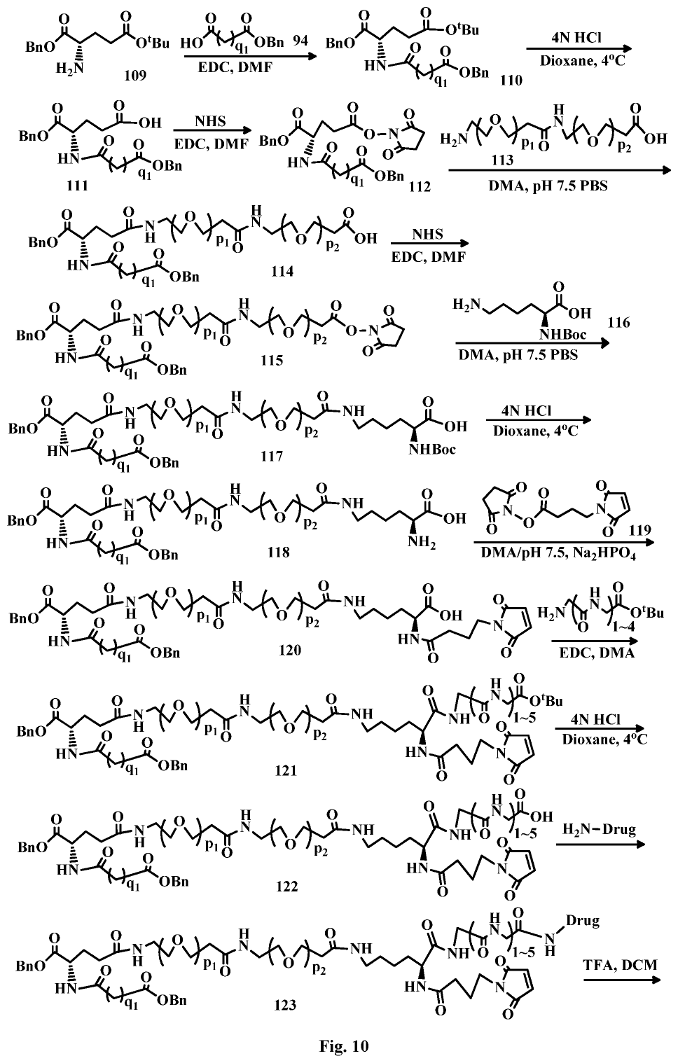

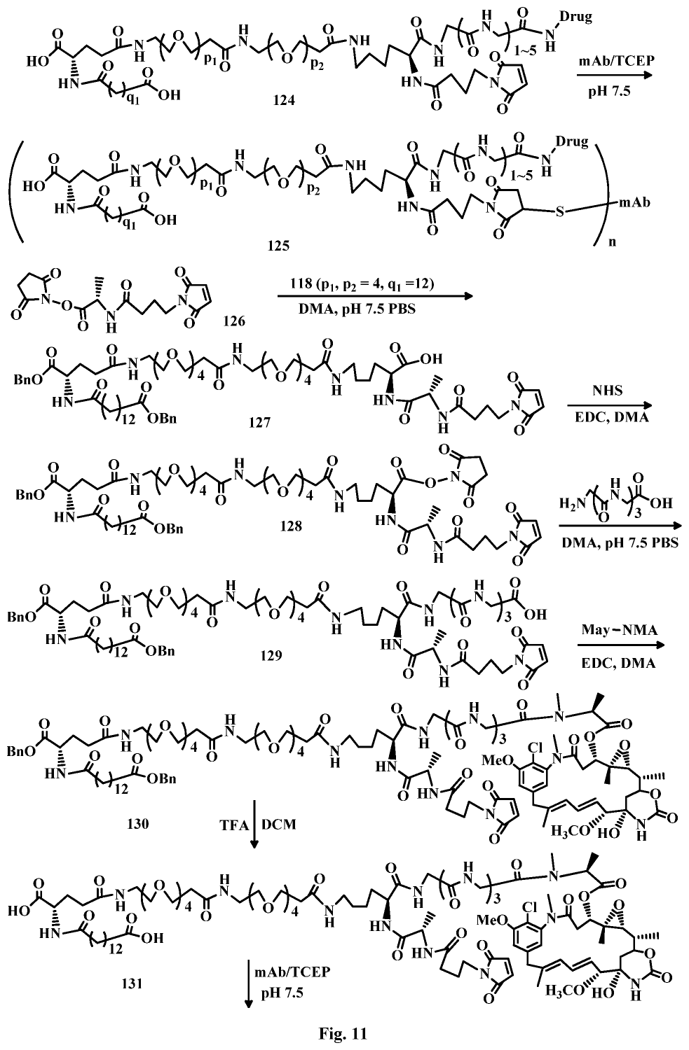

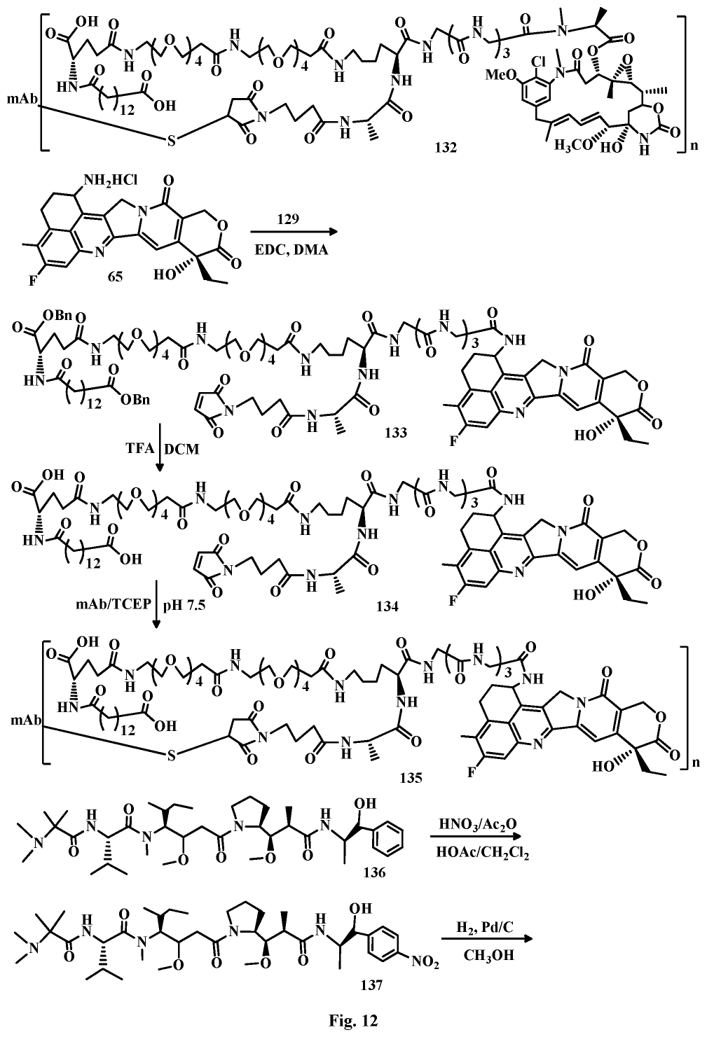

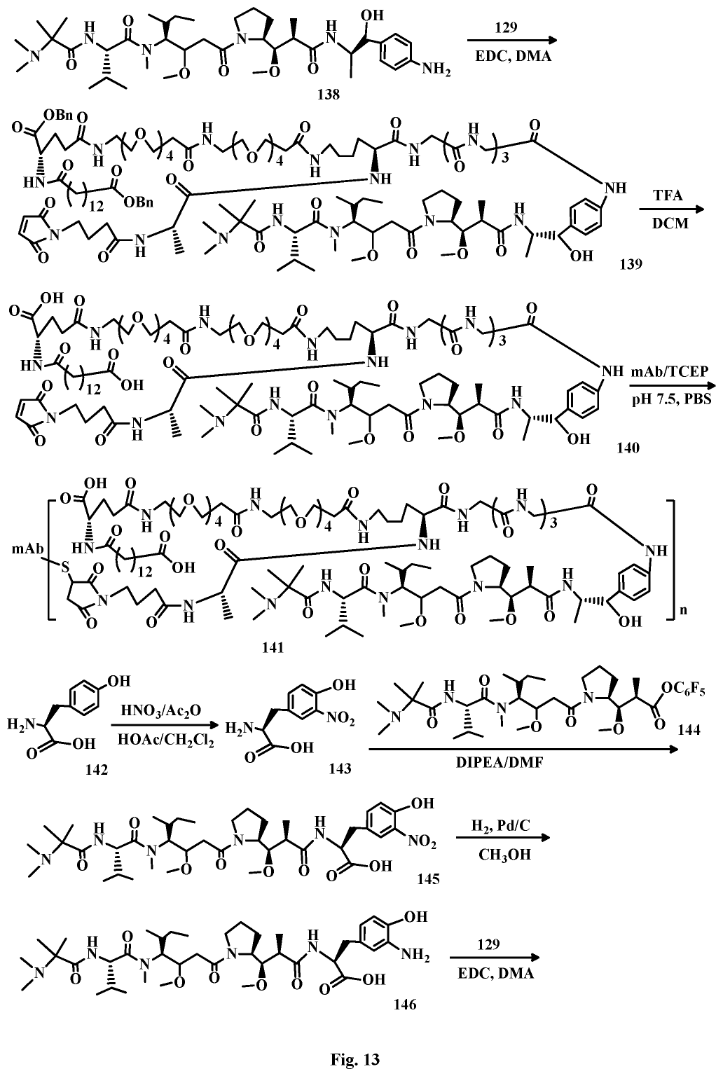

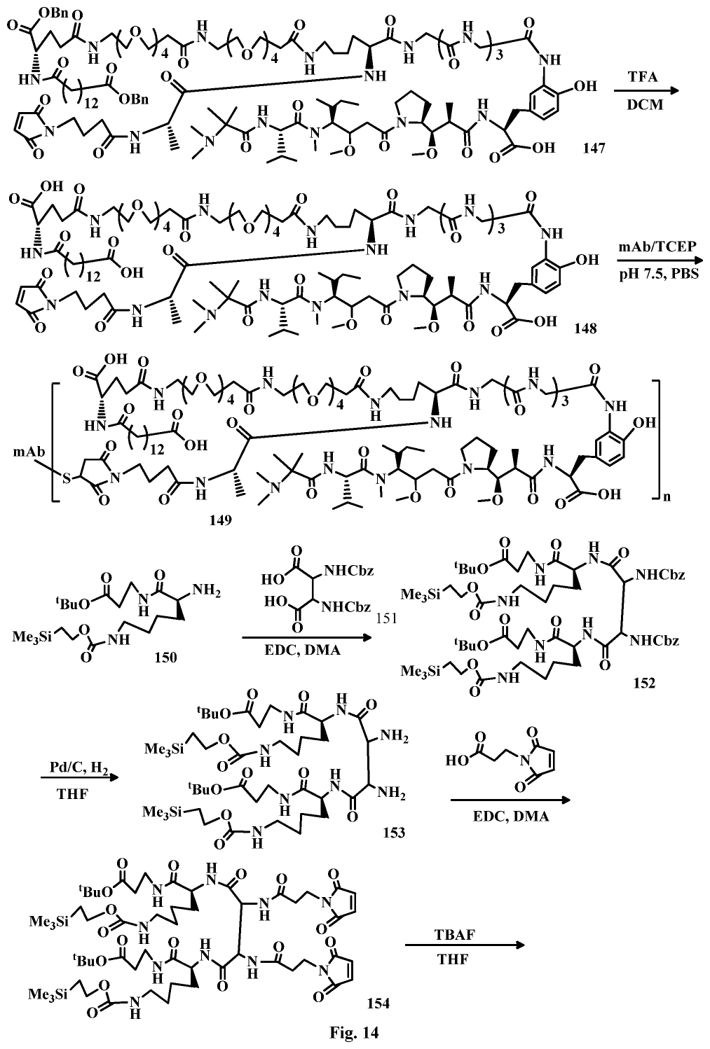

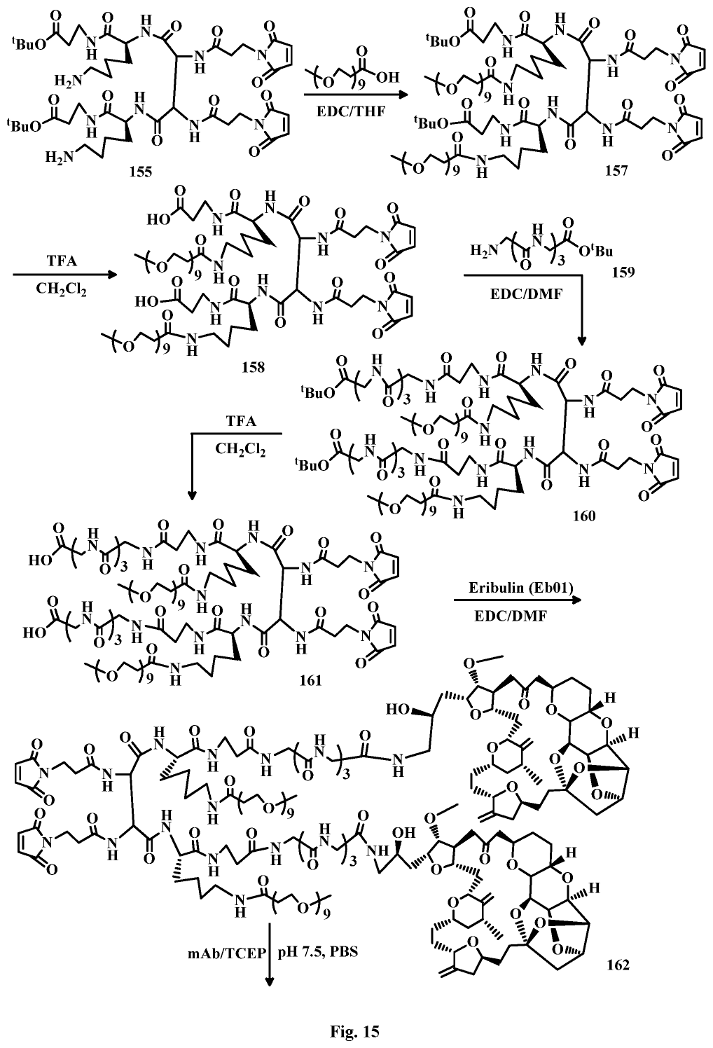

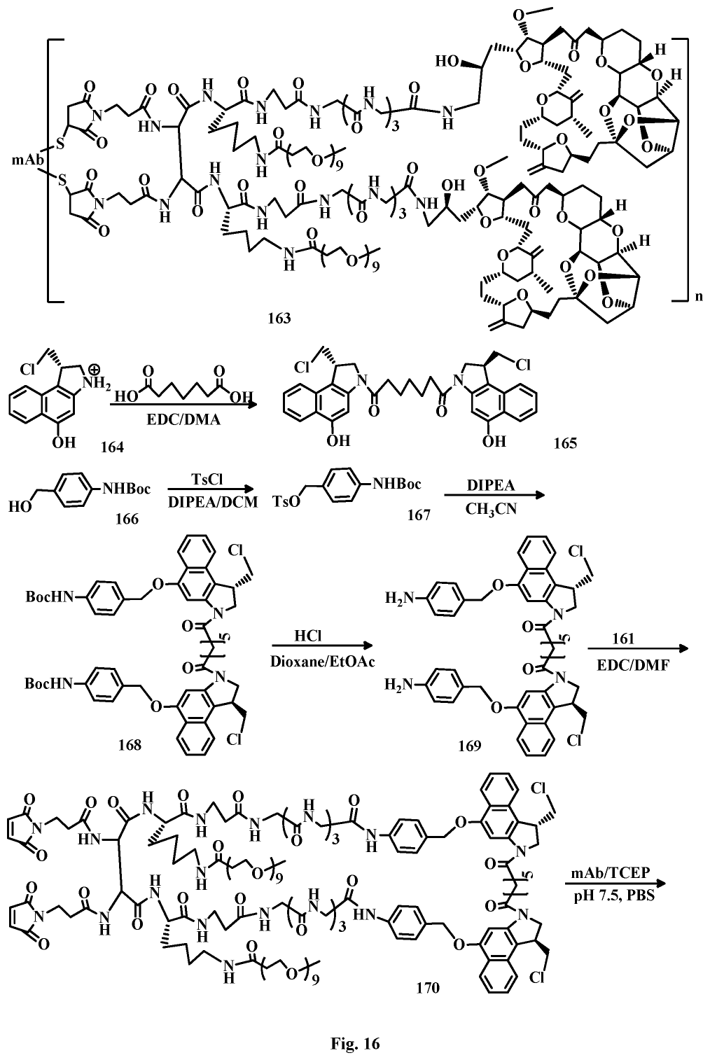

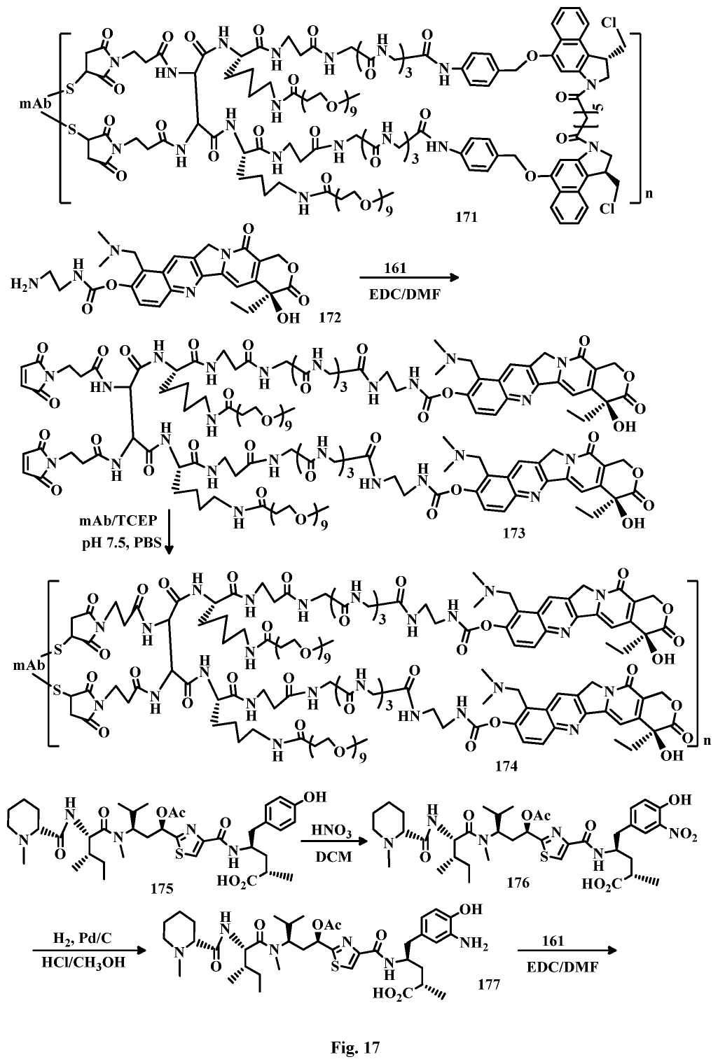

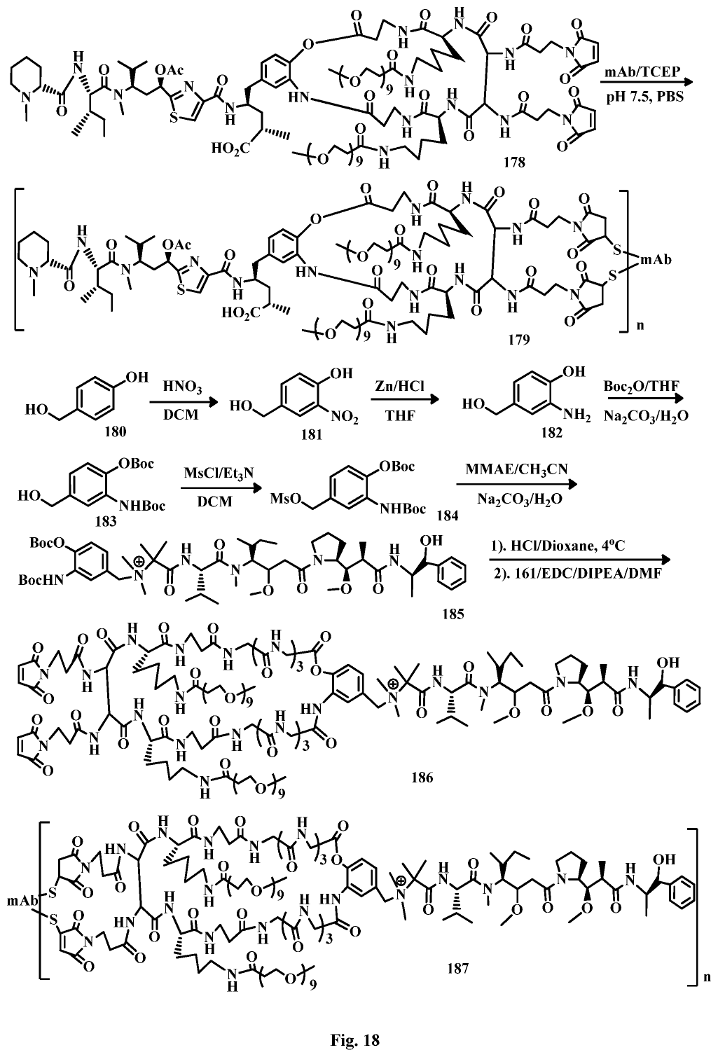

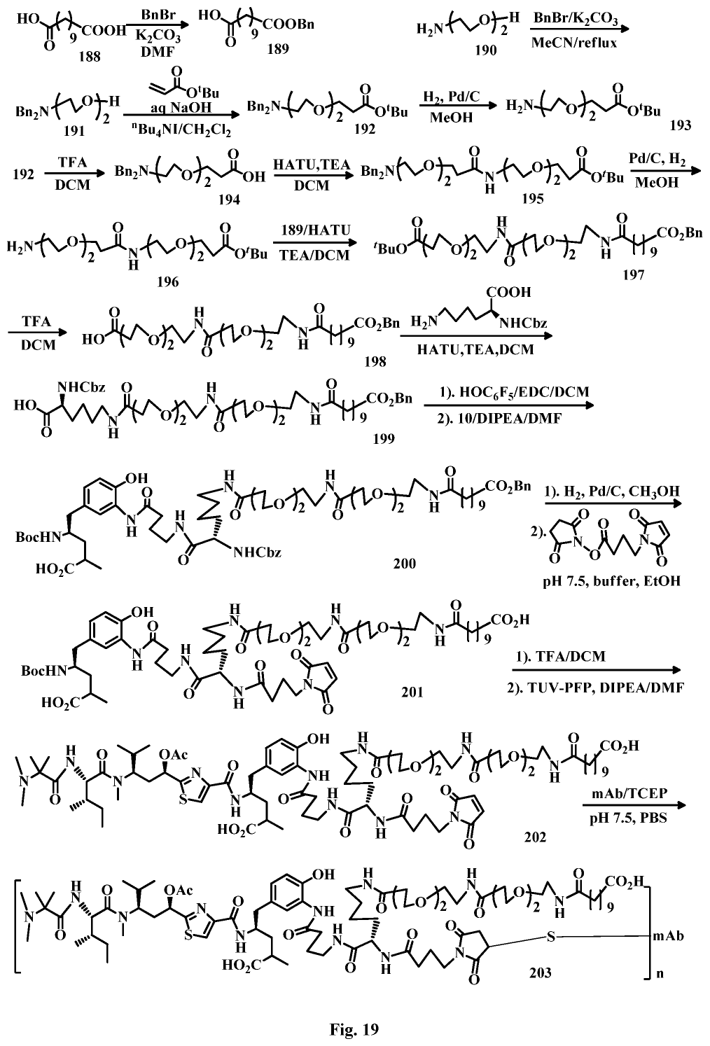

The novel conjugates disclosed herein use the bridge linkers. Examples of some suitable linkers and their synthesis are shown in FIGS. 1 to 26.

A Conjugate of a Cell-Binding Agent-A Cytotoxic Molecule Via the Side Chain-Linkage

In one aspect of the present invention, a conjugate containing a side chain-linkage is represented by Formula (I), (II), and (III):

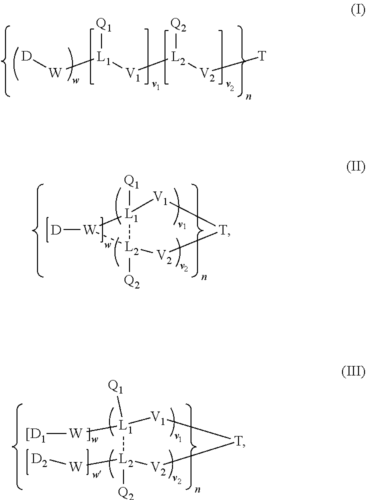

“ [symbol] ” represents a single bond; [symbol] is a single bond, double bond or absent; n is 1 to 30; w and w′ are independently 1, 2 or 3; and.

T is a cell-binding agent/molecule, selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and/or a cell-binding peptide, protein, or small molecule attached on albumin, a polymer, a dendrimer, a liposome, a nanoparticle, a vesicle, or on a (viral) capsid;

L₁ and L₂ are a chain of atoms selected from C, N, O, S, Si, and P, preferably having 0˜500 atoms, which covalently connects to W and V₁, and V₁ and V₂. The atoms used in forming the L₁ and L₂ may be combined in all chemically relevant ways, such as forming alkylene, alkenylene, and alkynylene, ethers, polyoxyalkylene, esters, amines, imines, polyamines, hydrazines, hydrazones, amides, ureas, semicarbazides, carbazides, alkoxyamines, alkoxylamines, urethanes, amino acids, peptides, acyloxylamines, hydroxamic acids, or combination above thereof. Preferably L₁ and L₂ are, the same or different, independently selected from O, NH, N, S, P, NNH, NHNH, N(R₃), N(R₃)N(R_{3′}), CH, CO, C(O)NH, C(O)O, NHC(O)NH, NHC(O)O, polyethyleneoxy unit of formula (OCH₂CH₂)ₚOR₃, or (OCH₂CH—(CH₃))ₚOR₃, or NH(CH₂CH₂O)ₚR₃, or NH(CH₂CH(CH₃)O)ₚR₃, or N[(CH₂CH₂O)ₚR₃]—[(CH₂CH₂O)_{p′}R_{3′}], or (OCH₂CH₂)ₚCOOR₃, or CH₂CH₂(OCH₂CH₂)ₚCOOR₃, wherein p and p′ are independently an integer selected from 0 to about 1000, or combination thereof; C₁-C₈ of alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or (Aa)ᵣ, r=1-12 (one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;

W is a stretcher unit having C₁-C₁₈, normally a self-immolative spacer, a peptidyl unit, a hydrazone, a disulfide, a thioether, an ester, or an amide bond;

V₁ and V₂ are independently a spacer unit and selected from O, NH, S, C₁-C₈ alkyl, C₂-C₈ heteroalkyl, alkenyl, or alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12 (one to 12 amino acid units), which is composed from a natural or unnatural amino acid, or the same or different sequences of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000; and v₁ and v₂ are independently 0, 1 or 2, but v₁ and v₂ are not 0 at the same time; when v₁ or v₂ is 0, it means one of the side chain Q₁ or Q₂ fragment is absent.

Q₁ and Q₂ are independently represented by Formula (I-q1):

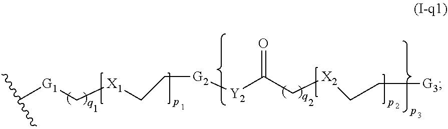

wherein [symbol] is the site linked to L₁ or L₂; G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1}; G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH₂, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing the same or different sequence of natural or unnatural amino acids; X₁ and X₂ are independently O, CH₂, S, S(O), NHNH, NH, N(R₁₂), ⁺NH(R₁₂), ⁺N(R₁₂)(R_{12′}), C(O), OC(O), OC(O)O, OC(O)NH, NHC(O)NH; Y₂ is O. NH, NR₁₂, CH₂. S, NHNH, Ar; p₁, p₂ and p₃ are independently 0-100 but are not 0 at the same time; q₁ and q₂ are independently 0-24; R₁₂, R_{12′}, R₁₃ and R_{13′}, are independently H, C₁˜C₈ alkyl; C₂˜C₈ heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl;

Preferably Q₁ and Q₂ are independently a C₂-C₁₀₀ polycarboxylacid, a C₂-C₉₀ polyalkylamine, a C₆-C₉₀ oligosaachride or polysaccharide, a C₆-C₁₀₀ zwitterionic betaines or zwitterionic poly(sulfobetaine)) (PSB)s that consist of a quarternary ammonium cation and/or a sulfonate anion, a C₆-C₁₀₀ biodegradable polymer, such as composed of poly (lactic/glycolic acid) (PLGA), poly(acrylates), chitosans, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), Poly(ethylene glycol)(PEG), poly(propylene glycol)(PPG), poly(lactide-co-glycolide), poly(ethylene glycol)-modified peptides, poly(ethylene glycol)-containing an amino acid or peptides, poly(ethylene glycol)-modified lipids, poly-glycine, poly-N-methyl-glycine, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified

alkylphosphate, or poly(ethylene glycol)-modified alkyl quarternary ammonium;

Example structures of Q₁ and Q₂ are shown below:

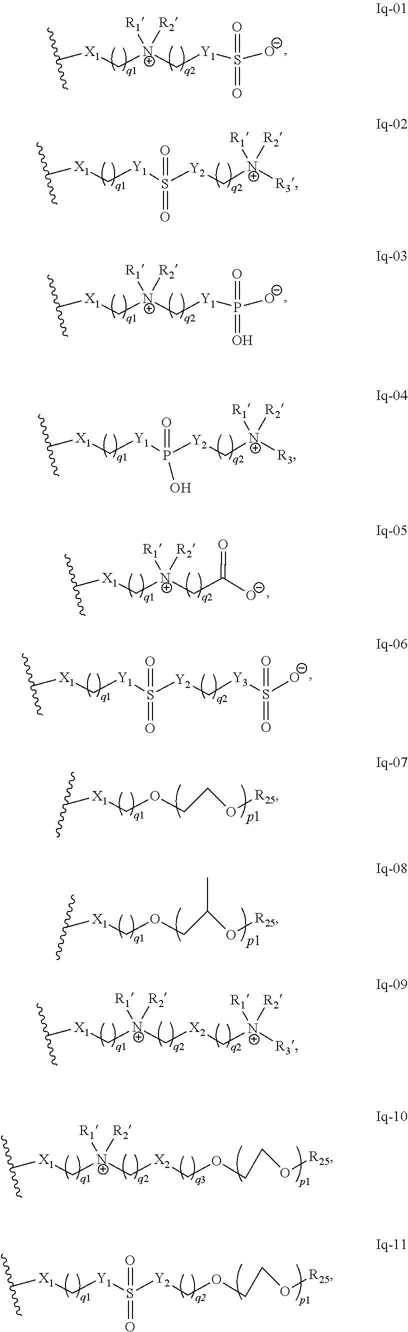

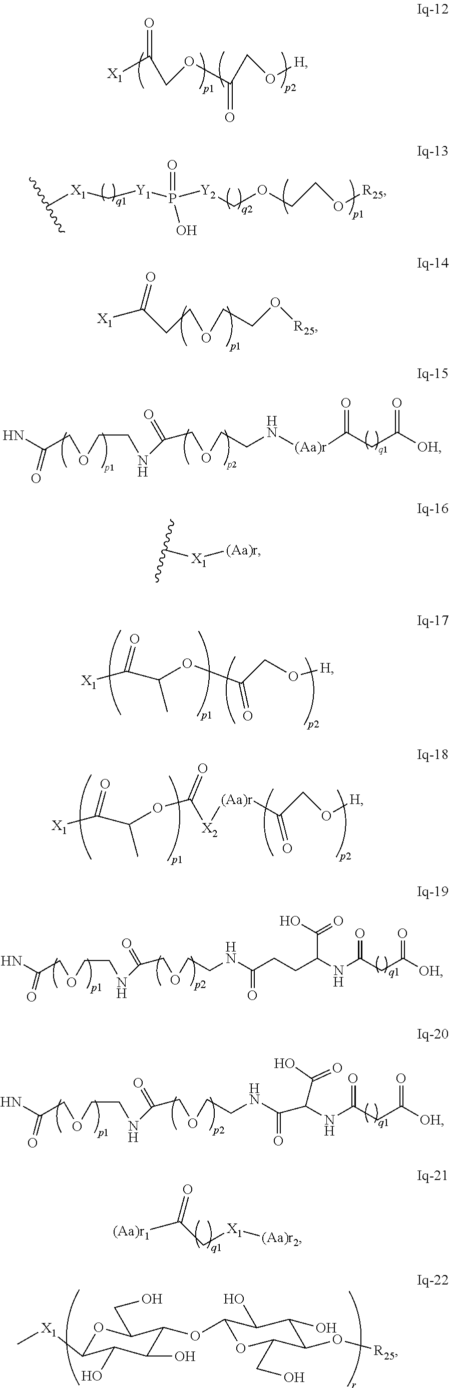

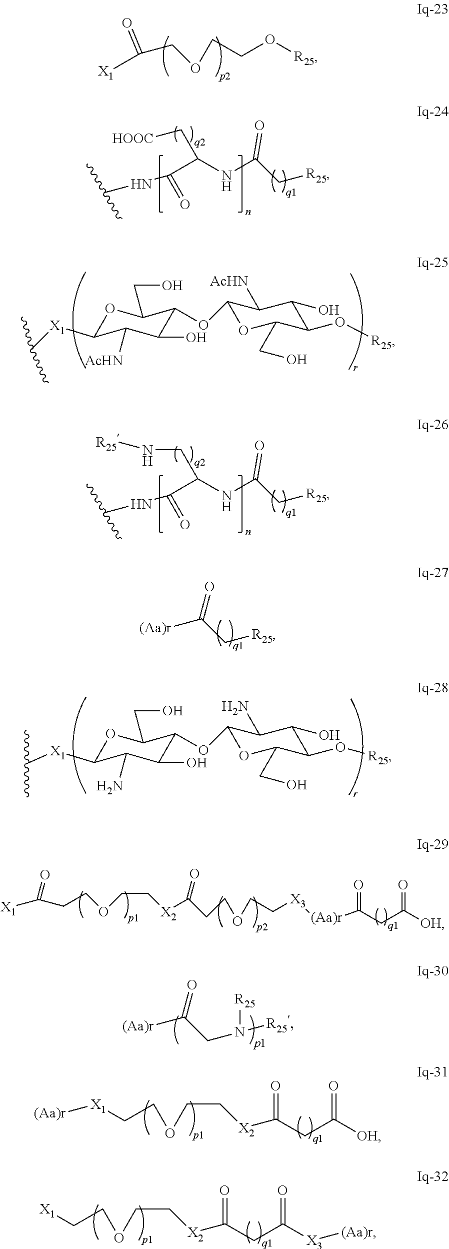

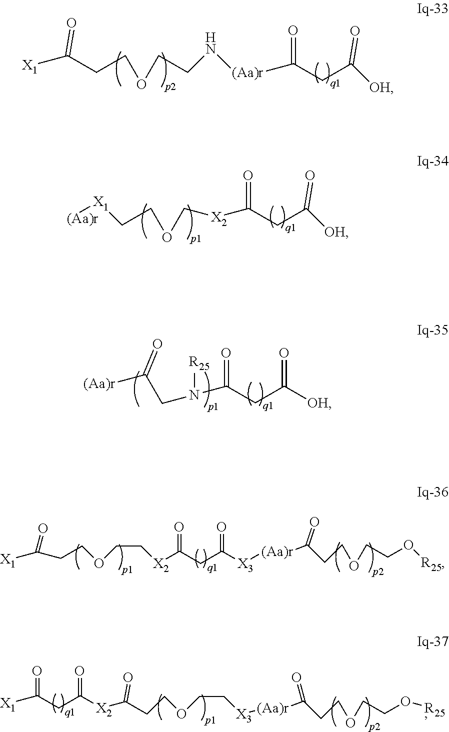

wherein R₂₅ and R_{25′} are independently selected from H; HC(O), CH₃C(O), CH₃C(NH), NHCH₃, COOH, CONH₂, CONHCH₃, C₁-C₁₈ alkyl, C₁-C₁₈ alkyl, alkyl-Y₁—SO₃H, C₁-C₁₈ alkyl-Y₁—PO₃H₂, C₁-C₁₈ alkyl-Y₁—CO₂H, C₁-C₁₈ alkyl-Y₁—N⁺R₁₂R₁₃R₁₃′R₁₄, C₁-C₁₈ alkyl-Y₁—CONH₂, C₂-C₁₈ alkylene, C₂-C₁₈ ester, C₂-C₁₈ ether, C₂-C₁₈ amine, C₂-C₁₈ alkyl carboxylamide, C₃-C₁₈ Aryl, C₃-C₁₈ cyclic alkyl, C₃-C₁₈ hyterocyclic, 1-24 amino acids; C₂-C₁₈ lipid, a C₂-C₁₈ fatty acid or a C₂-C₁₈ fatty ammonium lipid; X₁ and X₂ are independently selected from NH, N(R_{12′}), O, CH₂, S, C(O), S(O), S(O₂), P(O)(OH), NHNH, CH═CH, Ar or (Aa)q₁, q₁=0-24 (0-24 amino acids, q₁=0 means absent); X₁, X₂, X₃, X₄, Y₁, Y₂ and Y₃ are independently selected from NH, N(R₁₂′), O, C(O), CH₂, S, S(O), NHNH, C(O), OC(O), OC(O)O, OC(O)NH, NHC(O)NH, Ar or Ar or (Aa)q₁, X₁, X₂, X₃, X₄, Y₁, Y₂ and Y₃ can be independently absent; p₁, p₂ and p₃ are independently 0-100 but are not 0 at the same time; q₁, q₂ and q₃ are independently 0-24; R₁₂, R₁₃, R₁₃′ and R₁₄′ are independently selected from H and C₁-C₆ alkyl; Aa is natural or unnatural amino acid; Ar or (Aa)q₁, is the same or different sequence of peptides; q₁=0 means (Aa)q₁ absent; D is a cytotoxic agent that is independently selected from calicheamicins, maytansinoids, camptothecins, taxanes, anthracyclines (daunorubicin/doxorubicin), vinca alkaloids, auristatins, eribulins, (pyrrolo)benzodiazepines (PBDs), CC-106/duocarmycins, tubulysins, amatoxins (such as amanitins), protein kinase inhibitors, MEK inhibitors, KSP inhibitors, nicotinamide phosphoribosyltransferase (NAMPT) inhibitors, immunotoxins, analogs or prodrugs of these compounds above thereof; D₁ and D₂ are the same or different, and they are defined the same as D;

Calicheamicins and their related enediyne antibiotics that are described in: Nicolaou, K. C. et al, Science 1992, 256, 1172-1178; Proc. Natl. Acad. Sci USA. 1993, 90, 5881-8), U.S. Pat. Nos. 4,970,198; 5,053,394; 5,108,912; 5,264,586; 5,384,412; 5,606,040; 5,712,374; 5,714,586; 5,739,116; 5,770,701; 5,770,710; 5,773,001; 5,877,296; 6,015,562; 6,124,310; 8,153,768. The structure of calicheamicins is preferred the following formula:

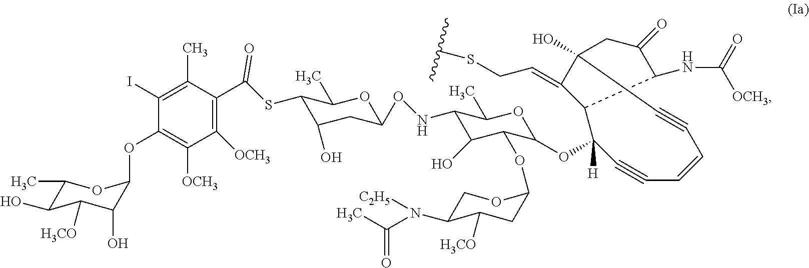 [SMILES: *SC/C=C1\[C@@H]2C(NC(=O)OC)C(=O)C[C@@]1(O)C#C/C=C\C#C[C@@H]2OC1OC(C)C(NOC2CC(O)C(SC(=O)c3c(C)c(I)c(OC4OC(C)C(O)C(OC)C4O)c(OC)c3OC)C(C)O2)C(O)C1OC1CC(OC)C(N(CC)C(C)=O)CO1]

or a isotope of a chemical element, or a pharmaceutically acceptable salt, hydrates, or hydrated salt; or a polymorphic crystalline structure; or an optical isomer, racemate, diastereomer or enantiomer thereof,

wherein [symbol] is the site linked to W;

Maytansinoids, including maytansinol and its analogues are described in U.S. Pat. Nos. 4,256,746, 4,361,650, 4,307,016, 4,294,757, 4,294,757, 4,371,533, 4,424,219, 4,331,598, 4,450,254, 4,364,866, 4,313,946, 4,315,929 4,362,663, 4,322,348, 4,371,533, 4,424,219, 5,208,020, 5,416,064, 5,208,020; 5,416,064; 6,333.410; 6,441,163; 6,716,821, 7,276,497, 7,301,019, 7,303,749, 7,368,565, 7,411,063, 7,851,432, and 8,163,888. The structure of maytansinoids is preferred the following formula:

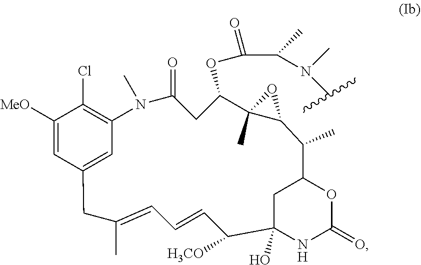

wherein [symbol] is the site linked to W.

A camptothecin (CPTs) and its derivatives, which are topoisomerase inhibitors to prevent DNA re-ligation and therefore to causes DNA damage resulting in apoptosis, are described in: Shang, X. F. et al, Med Res Rev. 2018, 38(3):775-828; Botella, P. and Rivero-Buceta, E. J Control Release. 2017, 247: 28-54; Martino, E. et al, Bioorg Med Chem Lett. 2017, 27(4):701-707; Lu, A., et al, Acta Pharmacol Sin 2007, 28(2): 307-314. It includes SN-38, Topotecan, Irinotecan (CPT-11), Silatecan (DB-67, AR-67), Cositecan (BNP-1350), Etirinotecan, Exatecan, Lurtotecan, Gimatecan (ST1481), Belotecan (CKD-602), Rubitecan and several others (Shang, X. F. et al, Med Res Rev. 2018, 38(3):775-828). So far three CPT analogues, topotecan, irinotecan, and belotecan have been approved and are used in cancer chemotherapy (Palakurthi, S., Expert Opin Drug Deliv. 2015; 12(12):1911-21; Shang, X. F. et al, Med Res Rev. 2018, 38(3):775-828) and both SN-38 and Exatecan have been successfully used as payloads for ADC conjugates in the clinical trials (Ocean, A. J. et al, Cancer. 2017, 123(19): 3843-3854; Starodub, A. N., et al, Clin Cancer Res. 2015, 21(17): 3870-8; Cardillo, T. M., et al, Bioconjug Chem. 2015, 26(5): 919-31; Ogitani, Y. et al, Bioorg Med Chem Lett. 2016, 26(20): 5069-5072; Takegawa, N. et al, Int J Cancer. 2017 Oct. 15; 141(8):1682-1689. U.S. Pat. Nos. 7,591,994; 7,999,083, 8,080,250, 8,268,317; US patent applications 20130090458, 20140099258, 20150297748, 20160279259).

The structure of Camptothecin (CPT) is illustrated below formula:

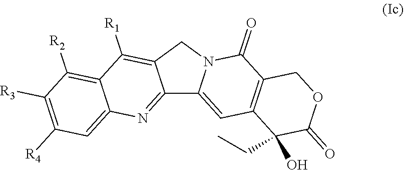

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R₁, R₂ and R₄ are independently selected from H, F, Cl, Br, CN, NO₂, C₁˜C₈ alkyl; O—C₁˜C₈ alkyl; NH—C₁˜C₈ alkyl; C₂-C₈ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 2-8 carbon atoms of esters, ether, amide, carbonate, urea, or carbamate; R₃ is H, OH, NH₂, C₁˜C₈ alkyl; O—C₁˜C₈ alkyl; NH—C₁˜C₈ alkyl; C₂-C₈ of heteroalkyl, alkylcycloalkyl, heterocycloalkyl; or 2-8 carbon atoms of esters, ether, amide, carbonate, urea, or carbamate; or R₁R₂, R₂R₃ and R₃R₄ independently form a 5˜7 membered carbocyclic, heterocyclic, heterocycloalkyl, aromatic or heteroaromatic ring system.

The structures of camptothecins are preferred the following formula:

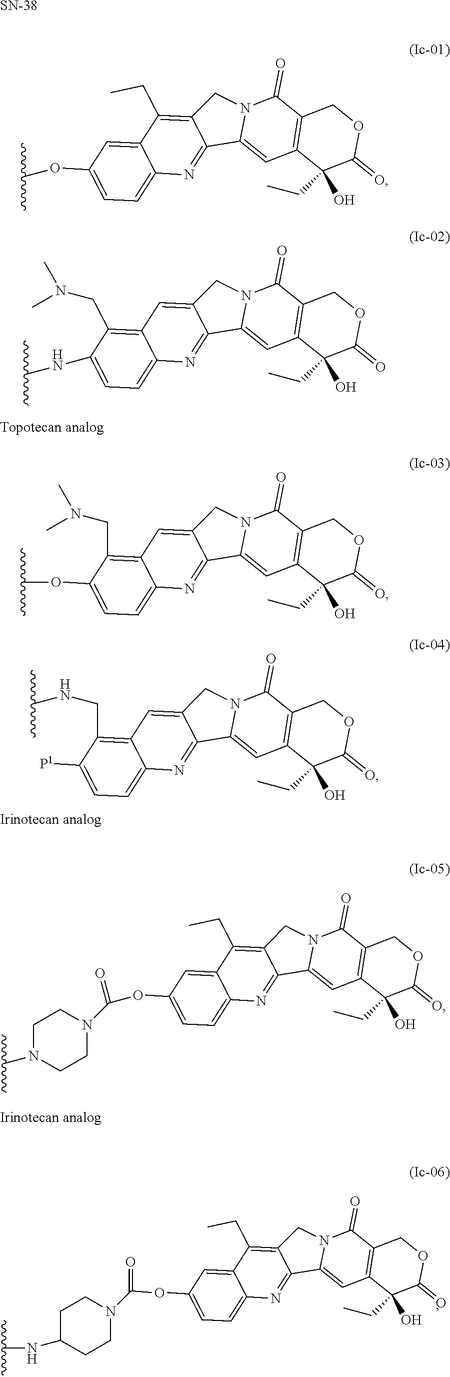

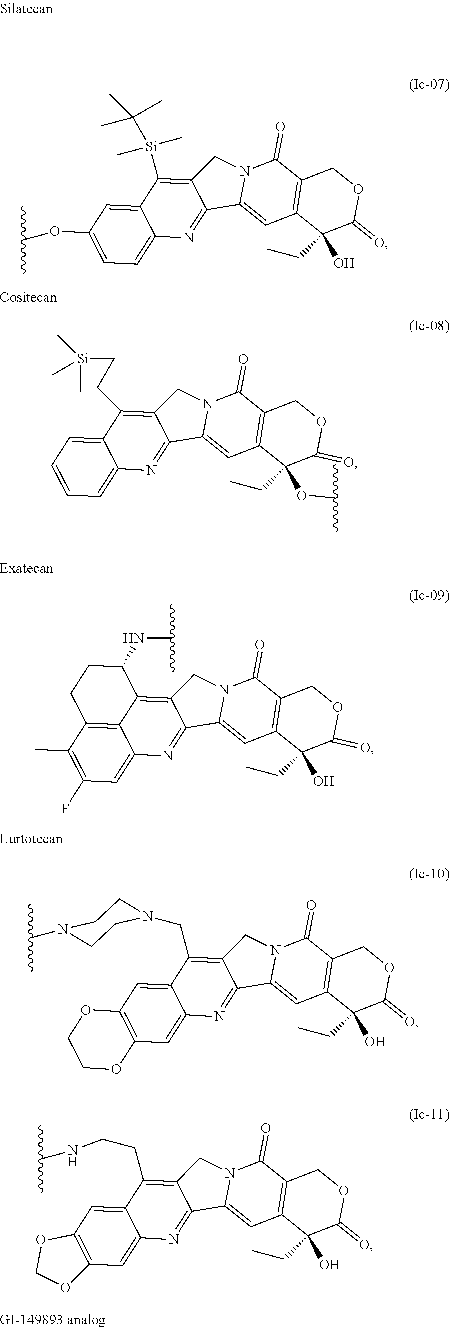 [SMILES: *Oc1ccc2nc3c(c([Si](C)(C)C(C)(C)C)c2c1)Cn1c-3cc2c(c1=O)COC(=O)[C@]2(O)CC ; *O[C@]1(CC)C(=O)OCc2c1cc1n(c2=O)Cc2c-1nc1ccccc1c2CC[Si](C)(C)C ; *N[C@H]1CCc2c(C)c(F)cc3nc4c(c1c23)Cn1c-4cc2c(c1=O)COC(=O)[C@]2(O)CC ; *N1CCN(Cc2c3c(nc4cc5c(cc24)OCCO5)-c2cc4c(c(=O)n2C3)COC(=O)[C@]4(O)CC)CC1 ; *NCCc1c2c(nc3cc4c(cc13)OCO4)-c1cc3c(c(=O)n1C2)COC(=O)[C@]3(O)CC]

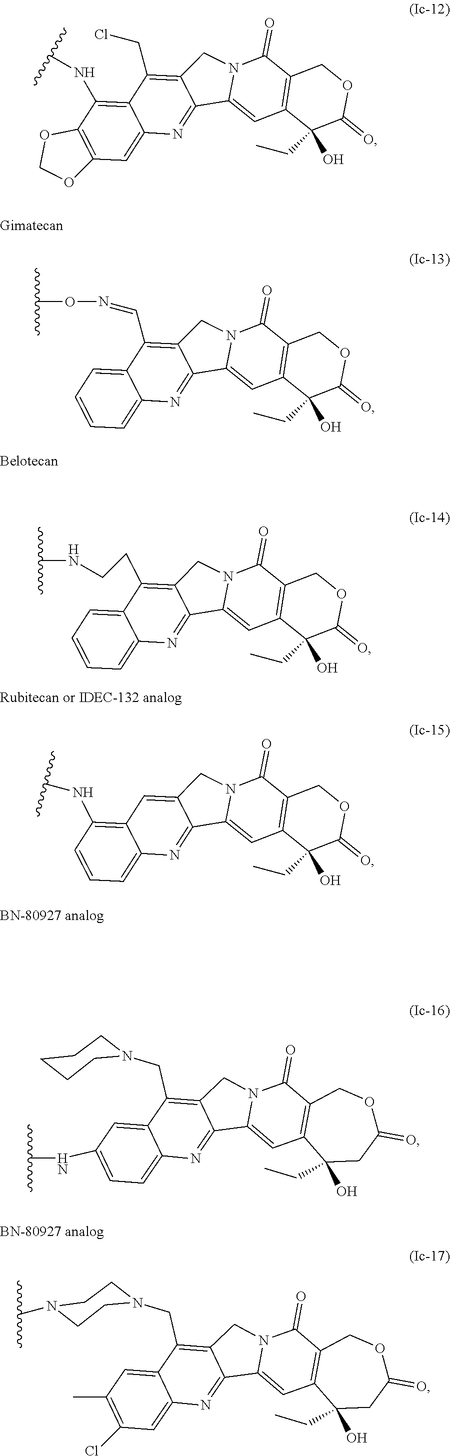 [SMILES: *Nc1c2c(cc3nc4c(c(CCl)c13)Cn1c-4cc3c(c1=O)COC(=O)[C@]3(O)CC)OCO2 ; *O/N=C\c1c2c(nc3ccccc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *Nc1cccc2nc3c(cc12)Cn1c-3cc2c(c1=O)COC(=O)[C@]2(O)CC ; *Nc1ccc2nc3c(c(CN4CCCCC4)c2c1)Cn1c-3cc2c(c1=O)COC(=O)C[C@]2(O)CC ; *N1CCN(Cc2c3c(nc4cc(Cl)c(C)cc24)-c2cc4c(c(=O)n2C3)COC(=O)C[C@]4(O)CC)CC1]

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein [symbol] is the connecting site linked to W; P¹ is H, OH, NH₂, COOH, C(O)NH₂, OCH₂OP(O)(OR¹⁸)₂, OC(O)OP(O)(OR¹⁸)₂, OPO(OR¹⁸)₂, NHPO(OR¹⁸)₂, OC(O)R¹⁸, OP(O)(OR¹⁸)OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, C₁-C₈ of linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸); R¹⁷ and R¹⁸ are independently H, linear or branched alkyl or heteroalkyl; C₂-C₈ of linear or branched alkenyl, alkynyl, alkylcycloalkyl, heterocycloalkyl; C₃-C₈ linear or branched of aryl, Ar-alkyl, heterocyclic, carbocyclic, cycloalkyl, heteroalkylcycloalkyl, alkylcarbonyl, heteroaryl; carbonate (—C(O)OR¹⁷), carbamate (—C(O)NR¹⁷R¹⁸).

Taxanes, which includes Paclitaxel (Taxol), a cytotoxic natural product, and docetaxel (Taxotere), a semi-synthetic derivative, and their analogs which are preferred for conjugation are exampled in: K C. Nicolaou et al., J. Am. Chem. Soc. 117, 2409-20, (1995); Ojima et al, J. Med. Chem. 39:3889-3896 (1996); 40:267-78 (1997); 45, 5620-3 (2002); Ojima et al., Proc. Natl. Acad. Sci., 96:4256-61 (1999); Kim et al., Bull. Korean Chem. Soc., 20, 1389-90 (1999); Miller, et al. J. Med. Chem., 47, 4802-5 (2004); U.S. Pat. No. 5,475,011 5,728,849, 5,811,452; 6,340,701; 6,372,738; 6,391,913, 6,436,931; 6,589,979; 6,596,757; 6,706,708; 7,008,942; 7,186,851; 7,217,819; 7,276,499; 7,598,290; and 7,667,054. The structures of taxanes are preferred the following formula:

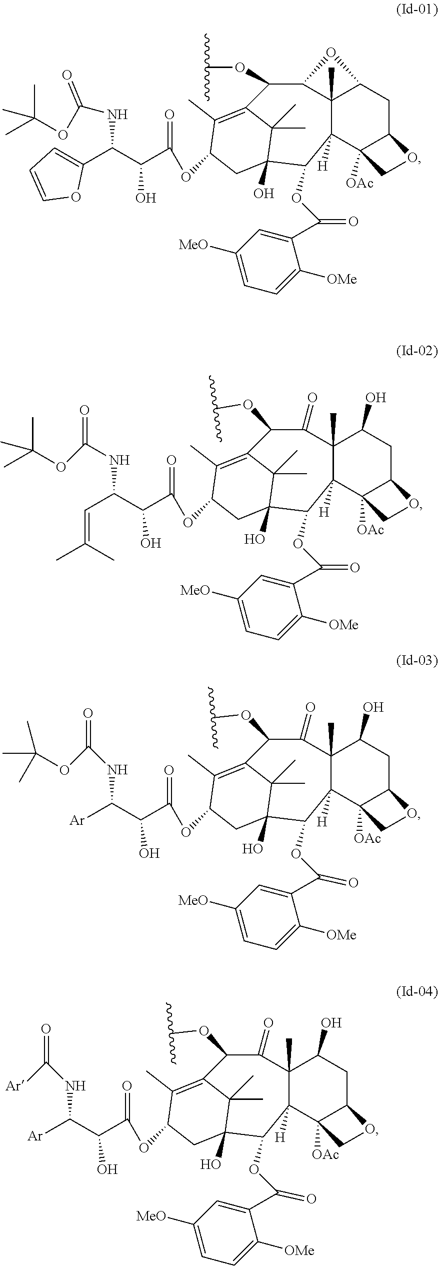

wherein [symbol] is the connecting site linked to W; Ar and Ar′ are independently aryl or heteroaryl.

Anthracyclines are mammalian DNA topoisomerases II inhibitors that are able to stabilize enzyme-DNA complexes wherein DNA strands are cut and covalently linked to the protein. These anticancer agents maintain a prominent role in treating many forms of solid tumors and acute leukemias during the last several decades. However, anthracyclines cause cardiovascular morbidity and mortality (Sagi, J. C., et al, Pharmacogenomics. 2016, 17(9), 1075-87; McGowan, J. V., et al, Cardiovasc Drugs Ther. 2017, 31(1), 63-75). Thus, to enhance specific activity of such molecules while reducing the cardiotoxicity, researchers actively are using the conjugation of anthracyclines to a cell-binding molecule as a general approach for improving the therapeutic index of these drugs, (Mollaev, M. et al, Int J Pharm. 2018 Dec. 29. pii: S0378-5173(18) 30991-8; Rossin, R., et al, Bioconjug Chem. 2016, 27(7):1697-706; Dal Corso, A., et al, J Control Release. 2017, 264:211-218). The structures of anthracyclines are preferred the following formula:

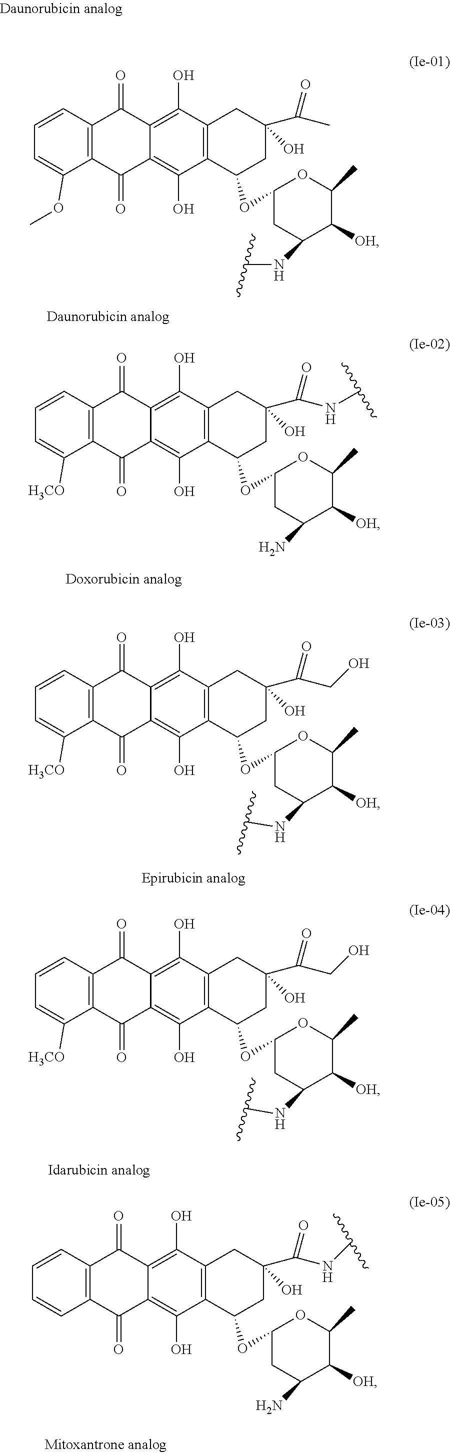

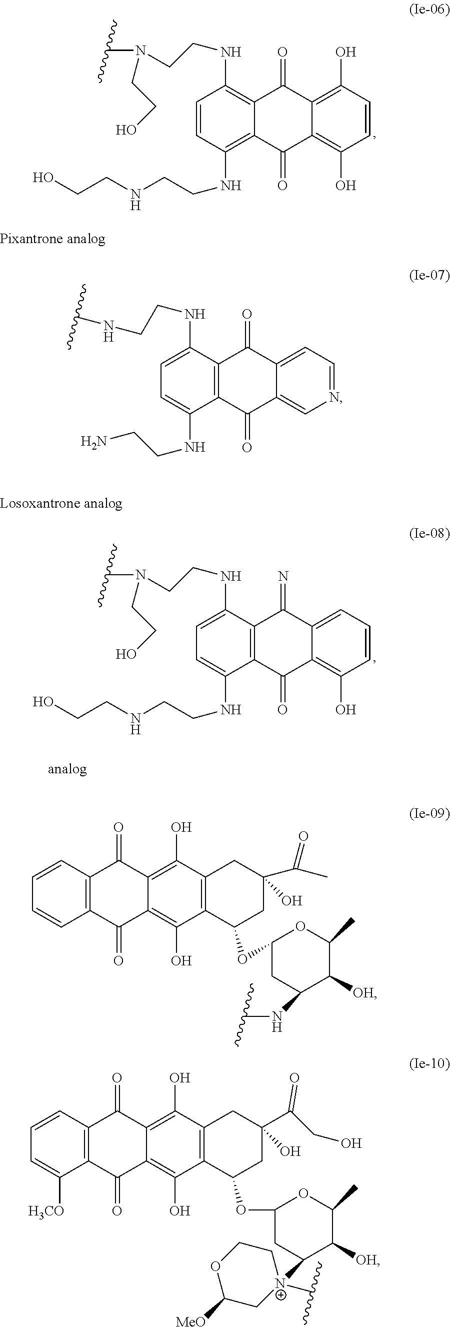

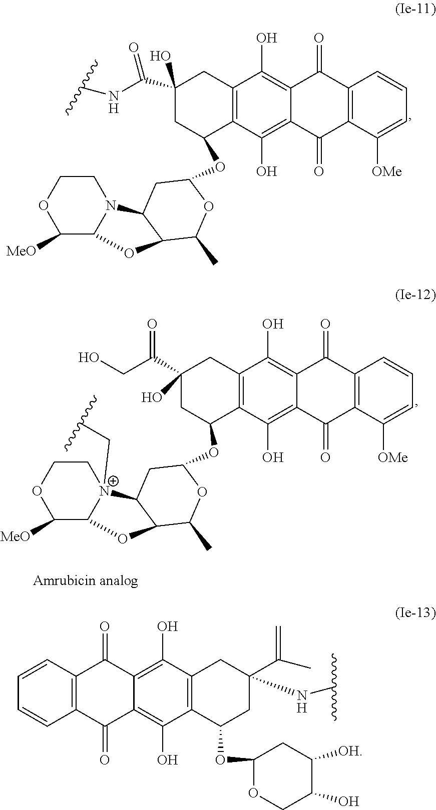

wherein [symbol] is the connecting site.

Vinca alkaloids are a set of anti-mitotic and anti-microtubule alkaloid agents that work by inhibiting the ability of cancer cells to divide. Vinca alkaloids include vinblastine, vincristine, vindesine, leurosine, vinorelbine, catharanthine, vindoline, vincaminol, vineridine, minovincine, methoxyminovincine, minovincinine, vincadifformine, desoxyvincaminol, vincamajine, vincamine, vinpocetine, and vinburnine. The structures of vinca alkaloids are preferred vinblastine, vincristine having the following formula:

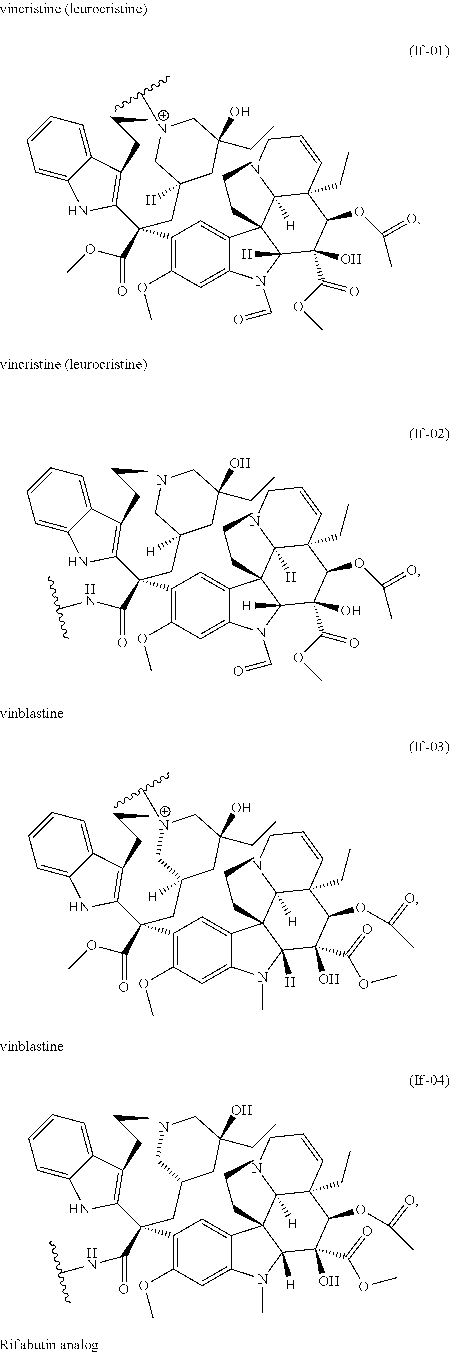

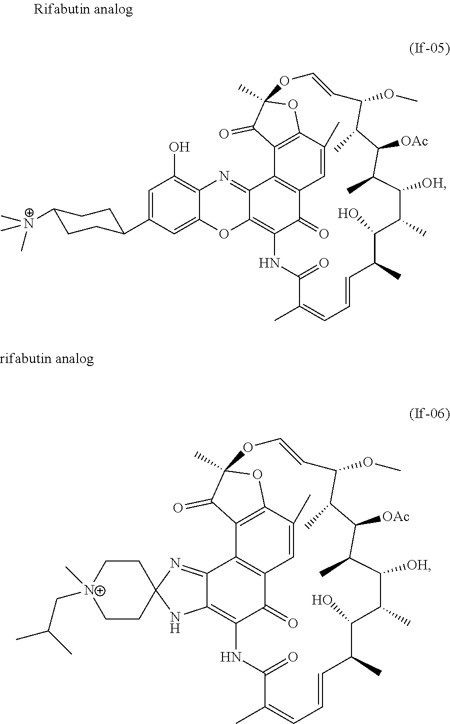 [SMILES: CO[C@H]1/C=C/O[C@@]2(C)Oc3c(C)cc4c(=O)c(c5oc6cc(C7CCC([N+](C)(C)C)CC7)cc(O)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C ; CO[C@H]1/C=C/O[C@@]2(C)Oc3c(C)cc4c(c3C2=O)C2=NC3(CC[N+](C)(CC(C)C)CC3)NC2=C(NC(=O)/C(C)=C\C=C\[C@H](C)[C@H](O)[C@@H](C)[C@@H](O)[C@@H](C)[C@H](OC(C)=O)[C@@H]1C)C4=O]

Auristatin or dolastatin analogs are preferred in conjugation containing the bis-linkers of this patent. The auristatins (e. g. auristatin E (AE) auristatin EB (AEB), auristatin EFP (AEFP), monomethyl auristatin E (MMAE), Monomethylauristatin (MMAF), Auristatin F phenylene diamine (AFP) and a phenylalanine variant of MMAE) which are synthetic analogs of dolastatins, are described in Int. J. Oncol. 15: 367-72 (1999); Molecular Cancer Therapeutics, vol. 3, No. 8, pp. 921-32 (2004); U.S. application Ser. Nos. 11/134,826, 20060074008, 2006022925. U.S. Pat. Nos. 4,414,205, 4,753,894, 4,764,368, 4,816,444, 4,879,278, 4,943,628, 4,978,744, 5,122,368, 5,165,923, 5,169,774, 5,286,637, 5,410,024, 5,521,284, 5,530,097, 5,554,725, 5,585,089, 5,599,902, 5,629,197, 5,635,483, 5,654,399, 5,663,149, 5,665,860, 5,708,146, 5,714,586, 5,741,892, 5,767,236, 5,767,237, 5,780,588, 5,821,337, 5,840,699, 5,965,537, 6,004,934, 6,033,876, 6,034,065, 6,048,720, 6,054,297, 6,054,561, 6,124,431, 6,143,721, 6,162,930, 6,214,345, 6,239,104, 6,323,315, 6,342,219, 6,342,221, 6,407,213, 6,569,834, 6,620,911, 6,639,055, 6,884,869, 6,913,748, 7,090,843, 7,091,186, 7,097,840, 7,098,305, 7,098,308, 7,498,298, 7,375,078, 7,462,352, 7,553,816, 7,659,241, 7,662,387, 7,745,394, 7,754,681, 7,829,531, 7,837,980, 7,837,995, 7,902,338, 7,964,566, 7,964,567, 7,851,437, 7,994,135. The structures of auristatin analogs are preferred the following formula (Ih-01), (Ih-02), (Ih-03), (Ih-04), (Ih-05), (Ih-06), and (Ih-07):

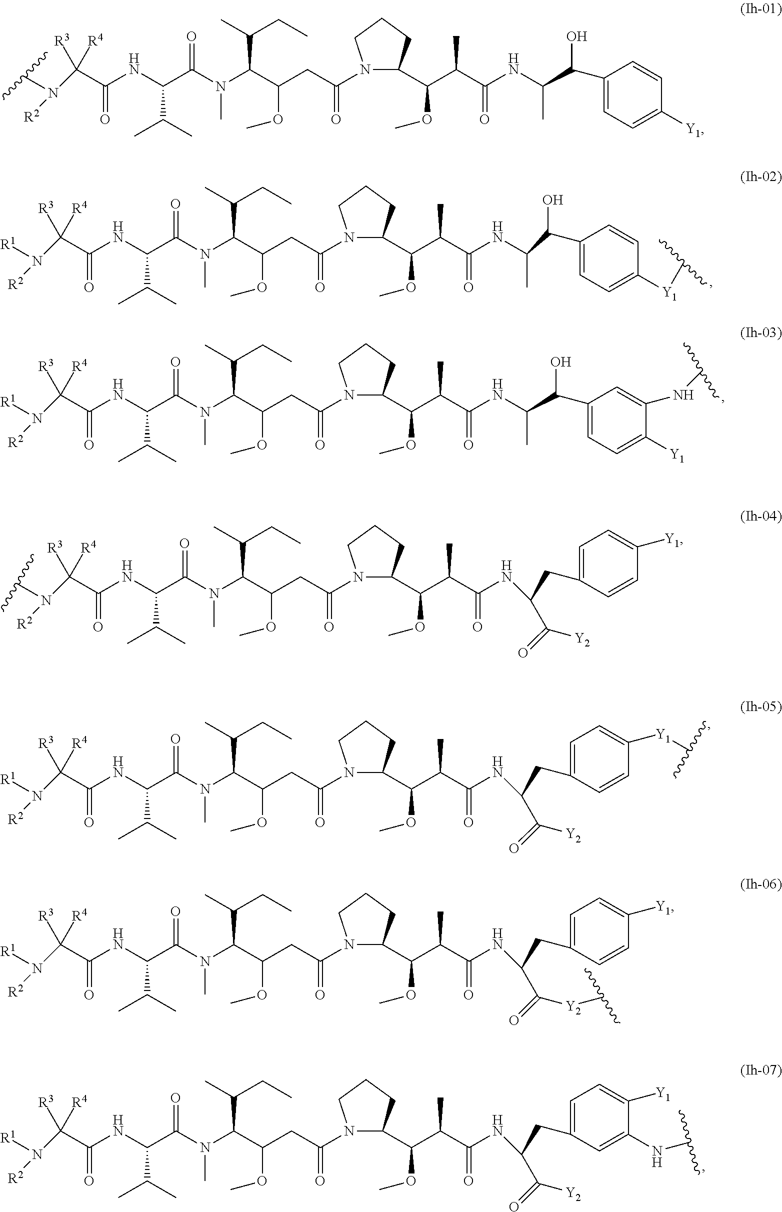

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein R¹, R², R³, R⁴ and R⁵ are independently H; C₁-C₈ lineal or branched alkyl, aryl, heteroaryl, heteroalkyl, alkylcycloalkyl, ester, ether, amide, amines, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000. The two Rs: R¹R², R²R³, R¹R³ or R³R⁴ can form 3˜8 member cyclic ring of alkyl, aryl, heteroaryl, heteroalkyl, or alkylcycloalkyl group; X₃ is H, CH₃ or X₁′R₁′, wherein X₁′ is NH, N(CH₃), NHNH, O, or S, and R₁′ is H or C₁-C₈ lineal or branched alkyl, aryl, heteroaryl, heteroalkyl, alkylcycloalkyl, acyloxylamines; R₃′ is H or C₁-C₆ lineal or branched alkyl; Z₃′ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃; Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting “ [symbol] ”; or OH, NH₂, NHNH₂, NHR₅, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different aminoacids; p is 1-5000; R₁, R₂, R₃, R₄, R₅, R₅′, Z₁, Z₂, and n are defined the same above.

Eribulin which is binding predominantly to a small number of high affinity sites at the plus ends of existing microtubules has both cytotoxic and non-cytotoxic mechanisms of action. Its cytotoxic effects are related to its antimitotic activities, wherein apoptosis of cancer cells is induced following prolonged and irreversible mitotic blockade (Kuznetsov, G. et al, Cancer Research. 2004, 64 (16): 5760-6; Towle, M. J, et al, Cancer Research. 2010, 71 (2): 496-505). In addition to its cytotoxic, antimitotic-based mechanisms, preclinical studies in human breast cancer models have shown that eribulin also exerts complex effects on the biology of surviving cancer cells and residual tumors that appear unrelated to its antimitotic effects. Eribulin has been approved by US FDA for the treatment of metastatic breast cancer who have received at least two prior chemotherapy regimens for late-stage disease, including both anthracycline- and taxane-based chemotherapies, as well as for the treatment of liposarcoma (a specific type of soft tissue sarcoma) that cannot be removed by surgery (unresectable) or is advanced (metastatic). Eribulin has been used as payload for ADC conjugates (US20170252458). The structure of Eribulin is preferred the following formula, Eb01:

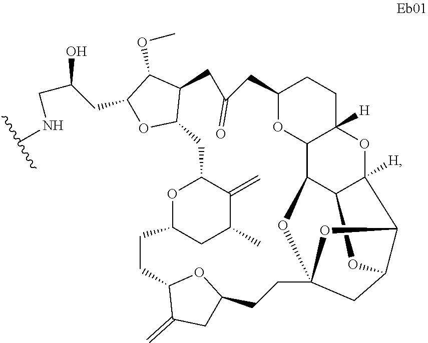

An Inhibitor of nicotinamide phosphoribosyltransferases (NAMPT) can be an interesting ADC payload due to their unique mechanisms of high potent activity (Sampath D, et al, Pharmacol Ther 2015; 151, 16-31). NAMPT regulates nicotinamide adenine dinucleotide (NAD) levels in cells wherein NAD plays as an essential redox cofactor to support energy and anabolic metabolism. NAD has several essential roles in metabolism. It acts as a coenzyme in redox reactions, as a donor of ADP-ribose moieties in ADP-ribosylation reactions, as a precursor of the second messenger molecule cyclic ADP-ribose, as well as acting as a substrate for bacterial DNA ligases and a group of enzymes called sirtuins that use NAD⁺ to remove acetyl groups from proteins. In addition to these metabolic functions, NAD⁺ emerges as an adenine nucleotide that can be released from cells spontaneously and by regulated mechanisms (Smyth L. M, et al, J. Biol. Chem. 2004, 279 (47), 48893-903; Billington R. A, et al, Mol Med. 2006, 12, 324-7), and can therefore have important extracellular roles (Billington R. A, et al, Mol Med. 2006, 12, 324-7). When inhibitors of NAMPT present, NAD levels decline below the level needed for metabolism resulting in energy crisis and therefore cell death. So far, clinical NAMPT inhibitor candidates FK-866, CHS-828, and GMX-1777 advanced to clinical trials but each encountered dose-limiting toxicities prior to any objective responses (Holen K., et al, Invest New Drugs 2008, 26, 45-51; Hovstadius, P., et al, Clin Cancer Res 2002, 8, 2843-50; Pishvaian, M. J., et al, J Clin Oncol 2009, 27, 3581). Thus using ADCs for targeting delivery of NAMPT inhibitors might circumvent the systemic toxicities to achieve much broader therapeutic index. The structures of NAMPT inhibitors are preferred the following formula, NP01, NP02, NP03, NP04, NP05, NP06, NP07, NP08, and NP09:

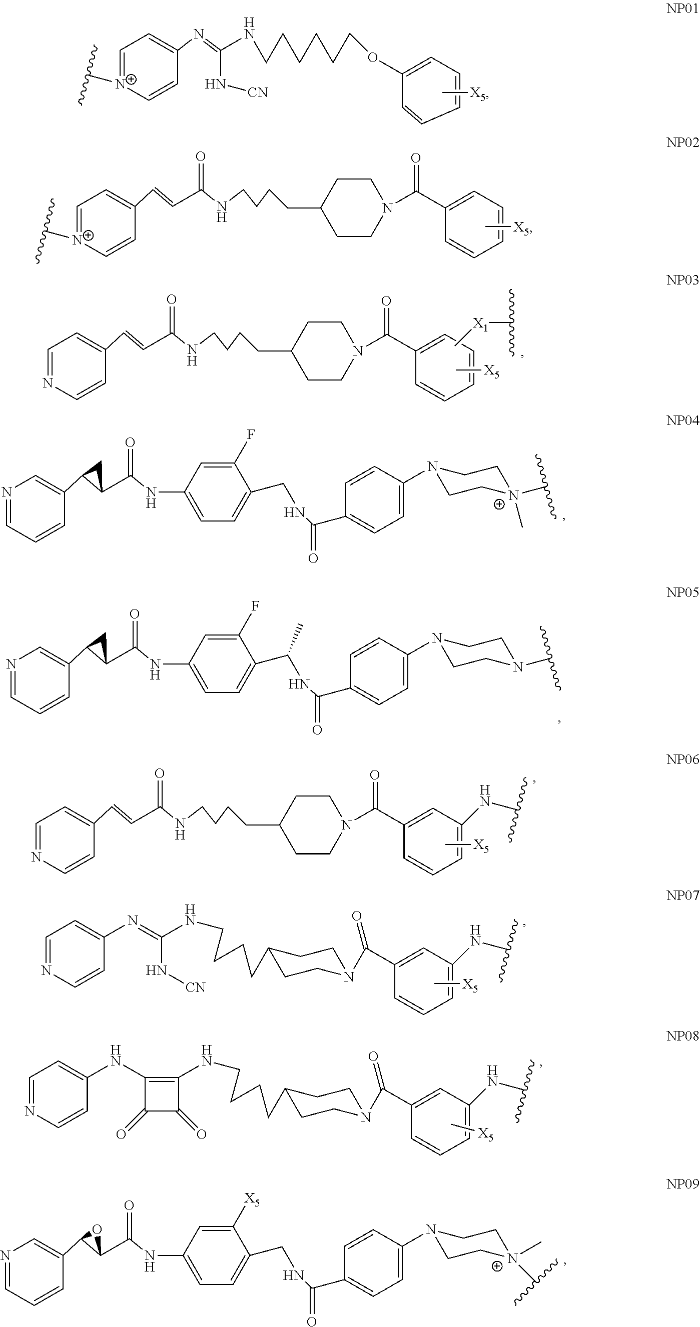

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein “ [symbol] ” is the same above; X₅ is F, Cl, Br, I, OH, OR₁, R₁, OPO₃H₂, OSO₃H, NHR₁, OCOR₁, NHCOR₁.

A benzodiazepine dimer and its analog: (e. g. a dimer of pyrrolobenzodiazepine (PBD) or (tomaymycin), indolinobenzodiazepine, imidazobenzothiadiazepine, or oxazolidinobenzodiazepines) which is preferred cytotoxic agent according to the present invention is exampled in: U.S. Pat. Nos. 8,163,736; 8,153,627; 8,034,808; 7,834,005; 7,741,319; 7,704,924; 7,691,848; 7,678,787; 7,612,062; 7,608,615; 7,557,099; 7,528,128; 7,528,126; 7,511,032; 7,429,658; 7,407,951; 7,326,700; 7,312,210; 7,265,105; 7,202,239; 7,189,710; 7,173,026; 7,109,193; 7,067,511; 7,064,120; 7,056,913; 7,049,311; 7,022,699; 7,015,215; 6,979,684; 6,951,853; 6,884,799; 6,800,622; 6,747,144; 6,660,856; 6,608,192; 6,562,806; 6,977,254; 6,951,853; 6,909,006; 6,344,451; 5,880,122; 4,935,362; 4,764,616; 4,761,412; 4,723,007; 4,723,003; 4,683,230; 4,663,453; 4,508,647; 4,464,467; 4,427,587; 4,000,304; US patent appl. 20100203007, 20100316656, 20030195196. Examples of the structures of the conjugate of the antibody-benzodiazepine dimers via the present linker are illustrated below PB01, PB02, PB03, PB04, PB05, PB06, PB07, PB08, PB09, PB10, PB11, PB12, PB13, PB14, PB15, and PB16.

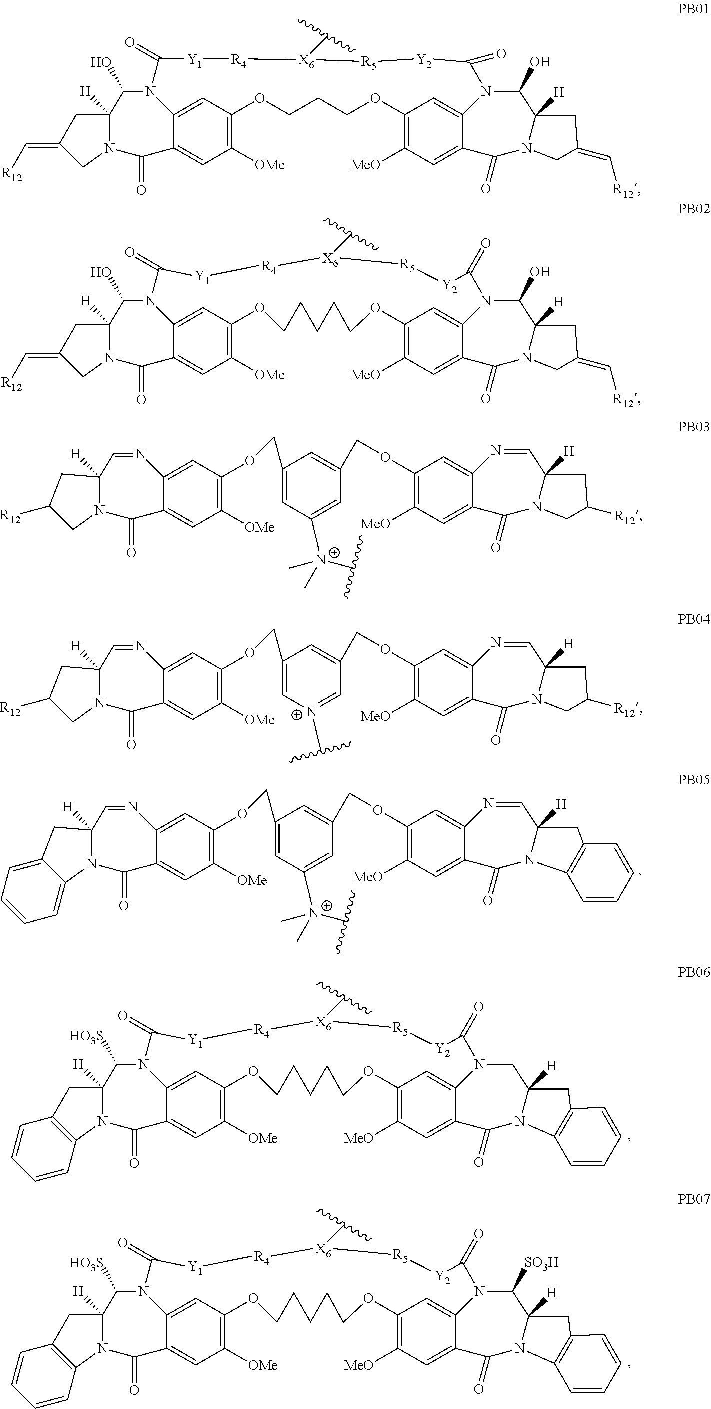

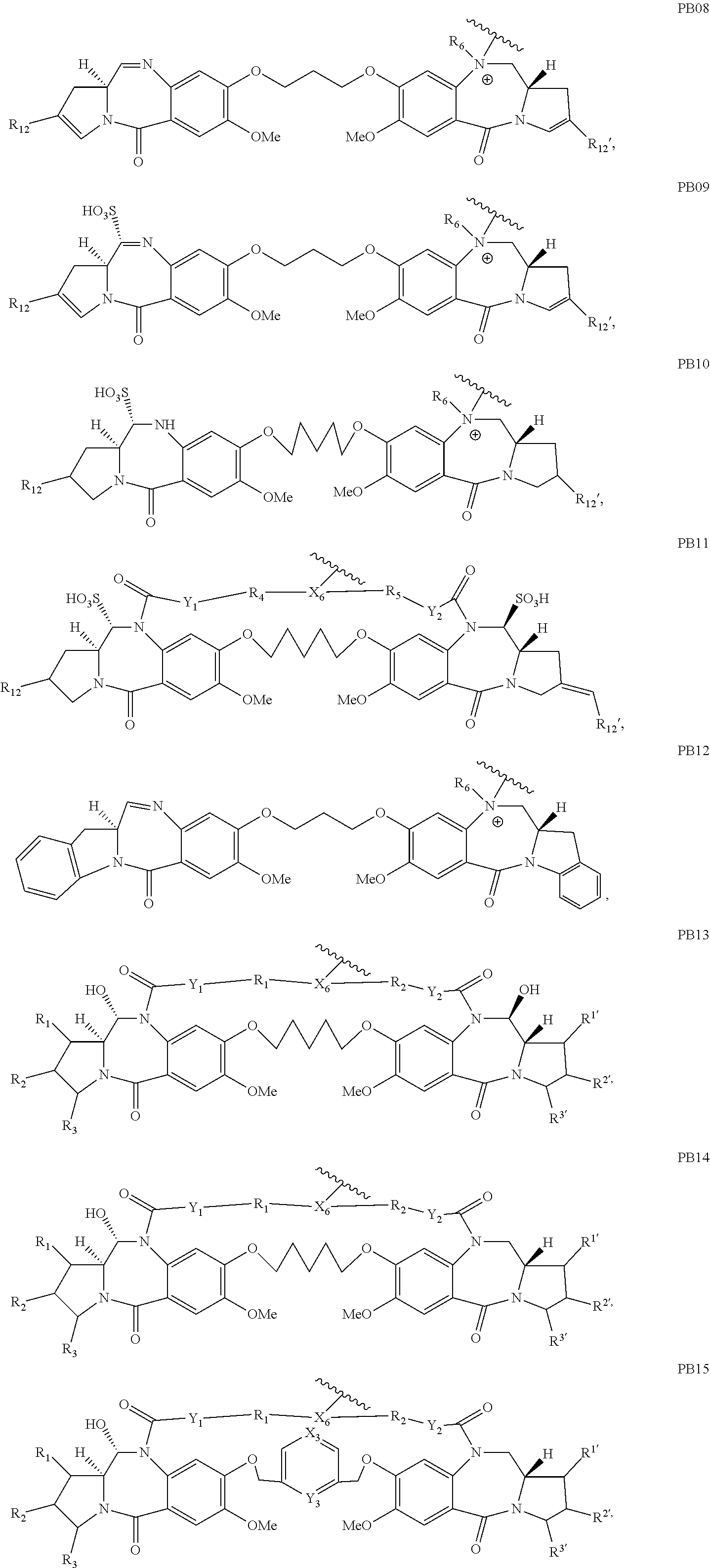

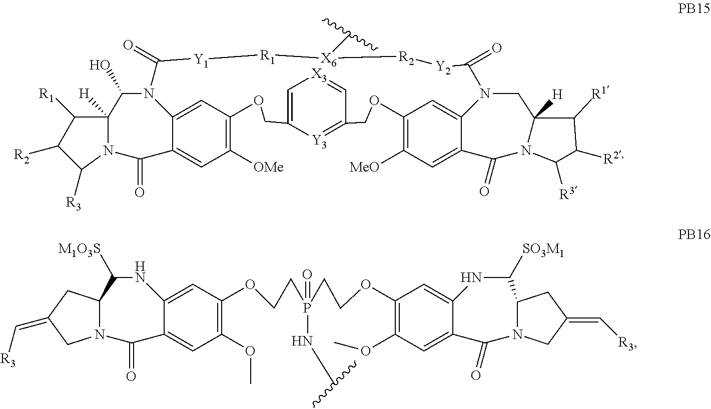

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, X₂, Y₁, Y₂, R₄, R₅, R₅′, Z₁, Z₂, and n are defined the same above; Preferably X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R¹, R², R³, R^{1′}, R^{2′}, and R^{3′} are independently H; F; Cl; ═O; ═S; OH; SH; C₁-C₈ lineal or branched alkyl, aryl, alkenyl, heteroaryl, heteroalkyl, alkylcycloalkyl, ester (COOR₅ or —OC(O)R₅), ether (OR₅), amide (CONR₅), carbamate (OCONR₅), amines (NHR₅, NR₅R₅′), heterocycloalkyl, or acyloxylamines (—C(O)NHOH, —ONHC(O)R₅); or peptides containing 1-20 natural or unnatural aminoacids, or polyethyleneoxy unit of formula (OCH₂CH₂)ₚ or (OCH₂CH(CH₃))ₚ, wherein p is an integer from 1 to about 5000. The two Rs: R¹R², R²R³, R¹R³, R^{1′}R^{2′}, R^{2′}R^{3′}, or R^{1′}R^{3′} can independently form 3˜8 member cyclic ring of alkyl, aryl, heteroaryl, heteroalkyl, or alkylcycloalkyl group; X₃ and Y₃ are independently N, NH, CH₂ or CR₅, wherein R₄, R₅, R₆, R₁₂ and R₁₂′ are independently H, OH, NH₂, NH(CH₃), NHNH₂, COOH, SH, OZ₃, SZ₃, F, Cl, or C₁-C₈ lineal or branched alkyl, aryl, heteroaryl, heteroalkyl, alkylcycloalkyl, acyloxylamines; Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside, etc.), NH-glycoside, S-glycoside or CH₂-glycoside; M₁ and M₂ are independently H, Na, K, Ca, Mg, NH₄, NR₁R₂R₃.

An CC-1065 analog and doucarmycin analogs are also preferred to be used for a conjugate containing bis-bridge linkage of the present patent. The examples of the CC-1065 analogues and doucarmycin analogs as well as their synthesis are described in: e.g. Warpehoski, et al, J. Med. Chem. 31:590-603 (1988); D. Boger et al., J. Org. Chem; 66; 6654-61, 2001; U.S. Pat. Nos. 4,169,888, 4,391,904, 4,671,958, 4,816,567, 4,912,227, 4,923,990, 4,952,394, 4,975,278, 4,978,757, 4,994,578, 5,037,993, 5,070,092, 5,084,468, 5,101,038, 5,117,006, 5,137,877, 5,138,059, 5,147,786, 5,187,186, 5,223,409, 5,225,539, 5,288,514, 5,324,483, 5,332,740, 5,332,837, 5,334,528, 5,403,484, 5,427,908, 5,475,092, 5,495,009, 5,530,101, 5,545,806, 5,547,667, 5,569,825, 5,571,698, 5,573,922, 5,580,717, 5,585,089, 5,585,499, 5,587,161, 5,595,499, 5,606,017, 5,622,929, 5,625,126, 5,629,430, 5,633,425, 5,641,780, 5,660,829, 5,661,016, 5,686,237, 5,693,762, 5,703,080, 5,712,374, 5,714,586, 5,739,116, 5,739,350, 5,770,429, 5,773,001, 5,773,435, 5,786,377 5,786,486, 5,789,650, 5,814,318, 5,846,545, 5,874,299, 5,877,296, 5,877,397, 5,885,793, 5,939,598, 5,962,216, 5,969,108, 5,985,908, 6,060,608, 6,066,742, 6,075,181, 6,103,236, 6,114,598, 6,130,237, 6,132,722, 6,143,901, 6,150,584, 6,162,963, 6,172,197, 6,180,370, 6,194,612, 6,214,345, 6,262,271, 6,281,354, 6,310,209, 6,329,497, 6,342,480, 6,486,326, 6,512,101, 6,521,404, 6,534,660, 6,544,731, 6,548,530, 6,555,313, 6,555,693, 6,566,336, 6,586,618, 6,593,081, 6,630,579, 6,756,397, 6,759,509, 6,762,179, 6,884,869, 6,897,034, 6,946,455, 7,049,316, 7,087,600, 7,091,186, 7,115,573, 7,129,261, 7,214,663, 7,223,837, 7,304,032, 7,329,507, 7,329,760, 7,388,026, 7,655,660, 7,655,661, 7,906,545, and 8,012,978. Examples of the structures of the conjugate of the antibody-CC-1065 analogs via the linker of the patent are illustrated below CC01, CC02, CC03, CC04, CC05, CC06 and CC07:

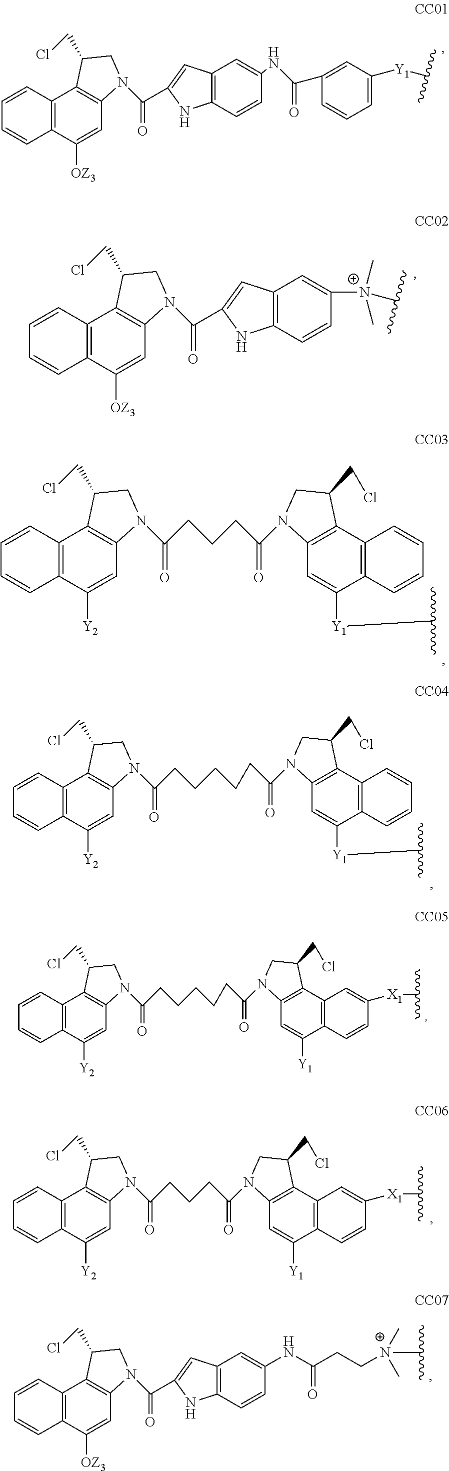

wherein X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) and C(O)NR₁ when linked to the connecting site “ [symbol] ”; or OH, NH₂, NHNH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH and C(O)NHR₁ when not linked to the connecting site “ [symbol] ”; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein R₁, R₂, R₃, M₁, M₂, and n are defined the same above;

A tubulysin and its analogs that are preferred for conjugation in the present invention are well known in the art and can be isolated from natural sources according to known methods or prepared synthetically according to known methods (e. g. Balasubramanian, R., et al. J. Med. Chem., 2009, 52, 238-40; Pando, O., et al. J. Am. Chem. Soc., 2011, 133, 7692-5; Reddy, J. A., et al. Mol. Pharmaceutics, 2009, 6, 1518-25; Raghavan, B., et al. J. Med. Chem., 2008, 51, 1530-33; Patterson, A. W., et al. J. Org. Chem., 2008, 73, 4362-9; Pando, O., et al. Org. Lett., 2009, 11 (24), 5567-9; Wipf, P., et al. Org. Lett., 2007, 9 (8), 1605-7; Peltier, H. M., et al. J. Am. Chem. Soc., 2006, 128, 16018-9; Chandrasekhar, S., et al J. Org. Chem., 2009, 74, 9531-4; Liu, Y., et al. Mol. Pharmaceutics, 2012, 9, 168-75; Friestad, G. K., et al. Org. Lett., 2009, 11, 1095-8; Kubicek, K., et al., Angew Chem Int Ed Engl, 2010.49: 4809-12; Chai, Y., et al., Chem Biol, 2010, 17: 296-309; Ullrich, A., et al., Angew Chem Int Ed Engl, 2009, 48, 4422-5; Sani, M., et al. Angew Chem Int Ed Engl, 2007, 46, 3526-9; Domling, A., et al., Angew Chem Int Ed Engl, 2006, 45, 7235-9; Patent applications: Zanda, M., et al, Can. Pat. Appl. CA 2710693 (2011); Chai, Y., et al. Eur. Pat. Appl. 2174947 (2010), WO 2010034724; Leamon, C. et al, WO2010033733, WO 2009002993; Ellman, J., et al, PCT WO2009134279; WO 2009012958, US appl. 20110263650, 20110021568; Matschiner, G., et al, WO2009095447; Vlahov, I., et al, WO2009055562, WO 2008112873; Low, P., et al, WO2009026177; Richter, W., WO2008138561; Kjems, J., et al, WO 2008125116; Davis, M.; et al, WO2008076333; Diener, J.; et al, U.S. Pat. Appl. 20070041901, WO2006096754; Matschiner, G., et al, WO2006056464; Vaghefi, F., et al, WO2006033913; Doemling, A., Ger. Offen. DE102004030227, WO2004005327, WO2004005326, WO2004005269; Stanton, M., et al, U.S. Pat. Appl. Publ. 20040249130; Hoefle, G., et al, Ger. Offen. DE10254439, DE10241152, DE10008089; Leung, D., et al, WO2002077036; Reichenbach, H., et al, Ger. Offen. DE19638870; Wolfgang, R., US20120129779; Chen, H., US appl. 20110027274. The preferred structures of tubulysins for conjugation of cell binding molecules are described in the patent application of PCT/IB2012/053554. Examples of the structures of the conjugates of the antibody-tubulysin analogs via the linker are Tb01, Tb02, Tb03, Tb04, Tb05, Tb06 Tb07, Tb08, Tb09, and T10 illustrated below:

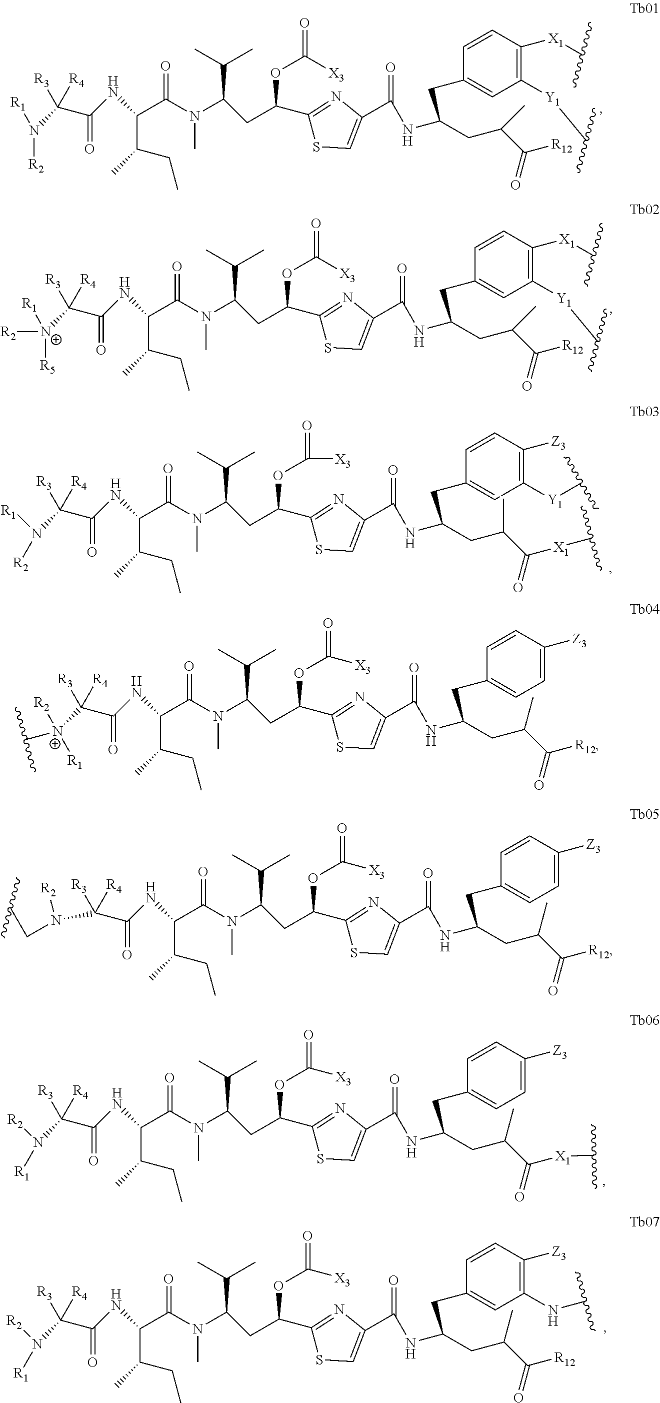

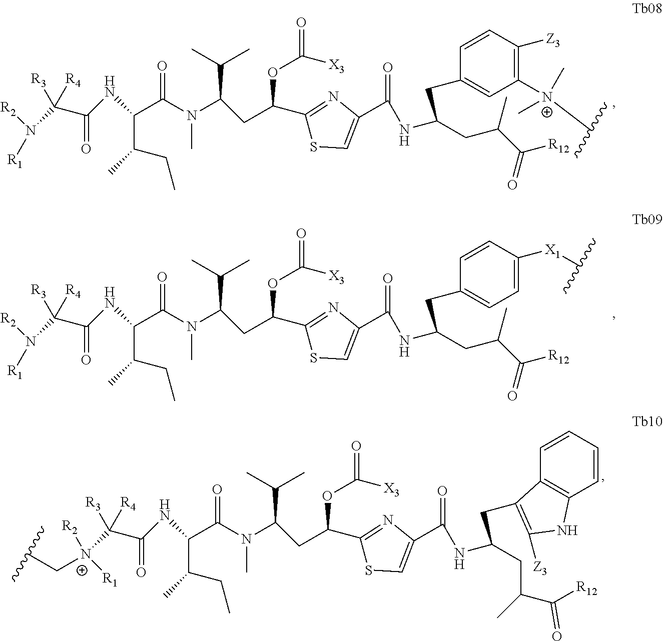

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; mAb is antibody, preferably monoclonal antibody; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂, wherein Aa is 1-8 aminoacids; n and mi are independently 1-20; p is 1-5000; Preferably R₁, R₁′, R₂, R₃, and R₄ are independently H, C₁-C₈ lineal or branched alkyl, amide, or amines; C₂-C₈ aryl, alkenyl, alkynyl, heteroaryl, heteroalkyl, alkylcycloalkyl, ester, ether, heterocycloalkyl, or acyloxylamines; or peptides containing 1-8 aminoacids, or polyethyleneoxy unit having formula (OCH₂CH₂)ₚ or (OCH₂CH(CH₃))ₚ, wherein p is an integer from 1 to about 5000; The two Rs: R₁R₂, R₂R₃, R₁R₃ or R₃R₄ can form 3˜8 member cyclic ring of alkyl, aryl, heteroaryl, heteroalkyl, or alkylcycloalkyl group; X₃ is H, CH₃, CH₂CH₃, C₃H₇, or X₁′R₁′, wherein X₁′ is NH, N(CH₃), NHNH, O, or S; R₁′ is H or C₁-C₈ lineal or branched alkyl, aryl, heteroaryl, heteroalkyl, alkylcycloalkyl, or acyloxylamines; R₃′ is H or C₁-C₆ lineal or branched alkyl; Z₃ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, O-glycoside (glucoside, galactoside, mannoside, glucuronoside/glucuronide, alloside, fructoside, etc), NH-glycoside, S-glycoside or CH₂-glycoside; M₁ and M₂ are independently H, Na, K, Ca, Mg, NH₄, NR₁R₂R₃; An amatoxin and its analogs which are a subgroup of at least ten toxic compounds originally found in several genera of poisonous mushrooms, most notably Amanita phalloides and several other mushroom species, are also preferred for conjugation of the present patent. These ten amatoxins, named α-Amanitin, β-Amanitin, γ-Amanitin, ε-Amanitin, Amanullin, Amanullinic acid, Amaninamide, Amanin, Proamanullin, are rigid bicyclic peptides that are synthesized as 35-amino-acid proproteins, from which the final eight amino acids are cleaved by a prolyl oligopeptidase (Litten, W. 1975 Scientific American232 (3): 90-101; H. E. Hallen, et al 2007 Proc. Nat. Aca. Sci. USA 104, 19097-101; K. Baumann, et al, 1993 Biochemistry 32 (15): 4043-50; Karlson-Stiber C, Persson H. 2003, Toxicon 42 (4): 339-49; Horgen, P. A. et al. 1978 Arch. Microbio. 118 (3): 317-9). Amatoxins kill cells by inhibiting RNA polymerase II (Pol II), shutting down gene transcription and protein biosynthesis (Brodner, O. G. and Wieland, T. 1976 Biochemistry, 15(16): 3480-4; Fiume, L., Curr Probl Clin Biochem, 1977, 7: 23-8; Karlson-Stiber C, Persson H. 2003, Toxicon 42(4): 339-49; Chafin, D. R., Guo, H. & Price, D. H. 1995 J. Biol. Chem. 270 (32): 19114-19; Wieland (1983) Int. J. Pept. Protein Res. 22(3): 257-76.). Amatoxins can be produced from collected Amanita phalloides mushrooms (Yocum, R. R. 1978 Biochemistry 17(18): 3786-9; Zhang, P. et al, 2005, FEMS Microbiol. Lett. 252(2), 223-8), or from fermentation using a basidiomycete (Muraoka, S. and Shinozawa T., 2000 J. Biosci. Bioeng. 89(1): 73-6) or from fermentation using A. fissa (Guo, X W., et al, 2006 Wei Sheng Wu Xue Bao 46(3): 373-8), or from culturing Galerina fasciculata or Galerina helvoliceps, a strain belonging to the genus (WO/1990/009799, JP11137291). However the yields from these isolation and fermentation were quite low (less than 5 mg/L culture). Several preparations of amatoxins and their analogs have been reported in the past three decades (W. E. Savige, A. Fontana, Chem. Commun. 1976, 600-1; Zanotti, G., et al, Int J Pept Protein Res, 1981. 18(2): 162-8; Wieland, T., et al, Eur. J. Biochem. 1981, 117, 161-4; P. A. Bartlett, et al, Tetrahedron Lett. 1982, 23, 619-22; Zanotti, G., et al., Biochim Biophys Acta, 1986. 870(3): 454-62; Zanotti, G., et al., Int. J. Peptide Protein Res. 1987, 30, 323-9; Zanotti, G., et al., Int. J. Peptide Protein Res. 1987, 30, 450-9; Zanotti, G., et al., Int J Pept Protein Res, 1988. 32(1): 9-20; G. Zanotti, T. et al, Int. J. Peptide Protein Res. 1989, 34, 222-8; Zanotti, G., et al., Int J Pept Protein Res, 1990. 35(3): 263-70; Mullersman, J. E. and J. F. Preston, 3rd, Int J Pept Protein Res, 1991. 37(6): 544-51; Mullersman, J. E., et al, Int J Pept Protein Res, 1991. 38(5): 409-16; Zanotti, G., et al, Int J Pept Protein Res, 1992. 40(6): 551-8; Schmitt, W. et al, J. Am. Chem. Soc. 1996, 118, 4380-7; Anderson, M. O., et al, J. Org. Chem., 2005, 70(12): 4578-84; J. P. May, et al, J. Org. Chem. 2005, 70, 8424-30; F. Brueckner, P. Cramer, Nat. Struct. Mol. Biol. 2008, 15, 811-8; J. P. May, D. M. Perrin, Chem. Eur. J. 2008, 14, 3404-9; J. P. May, et al, Chem. Eur. J. 2008, 14, 3410-17; Q. Wang, et al, Eur. J. Org. Chem. 2002, 834-9; May, J. P. and D. M. Perrin, Biopolymers, 2007. 88(5): 714-24; May, J. P., et al., Chemistry, 2008. 14(11): 3410-7; S. De Lamo Marin, et al, Eur. J. Org. Chem. 2010, 3985-9; Pousse, G., et al., Org Lett, 2010. 12(16): 3582-5; Luo, H., et al., Chem Biol, 2014. 21(12): 1610-7; Zhao, L., et al., Chembiochem, 2015. 16(10): 1420-5) and most of these preparations were by partial synthesis. Because of their extreme potency and unique mechanism of cytotoxicity, amatoxins have been used as payloads for conjugations (Fiume, L., Lancet, 1969. 2 (7625): 853-4; Barbanti-Brodano, G. and L. Fiume, Nat New Biol, 1973. 243(130): 281-3; Bonetti, E., M. et al, Arch Toxicol, 1976. 35(1): p. 69-73; Davis, M. T., Preston, J. F. Science 1981, 213, 1385-1388; Preston, J. F., et al, Arch Biochem Biophys, 1981. 209(1): 63-71; H. Faulstich, et al, Biochemistry 1981, 20, 6498-504; Barak, L. S., et al., Proc Natl Acad Sci USA, 1981. 78(5): 3034-8; Faulstich, H. and L. Fiume, Methods Enzymol, 1985. 112: 225-37; Zhelev, Z., A. et al, Toxicon, 1987. 25(9): 981-7; Khalacheva, K., et al, Eksp Med Morfol, 1990. 29(3): 26-30; U. Bermbach, H. Faulstich, Biochemistry 1990, 29, 6839-45; Mullersman, J. E. and J. F. Preston, Int. J. Peptide Protein Res. 1991, 37, 544-51; Mullersman, J. E. and J. F. Preston, Biochem Cell Biol, 1991. 69(7): 418-27; J. Anderl, H. Echner, H. Faulstich, Beilstein J. Org. Chem. 2012, 8, 2072-84; Moldenhauer, G., et al, J. Natl. Cancer Inst. 2012, 104, 622-34; A. Moshnikova, et al; Biochemistry 2013, 52, 1171-8; Zhao, L., et al., Chembiochem, 2015. 16(10): 1420-5; Zhou, B., et al., Biosens Bioelectron, 2015. 68: 189-96; WO2014/043403, US20150218220, EP 1661584). We have been working on the conjugation of amatoxins for a while. Examples of the structures of the conjugate of the antibody-amatoxins via the linker are preferred the following structures of Am01, Am02, and Am03:

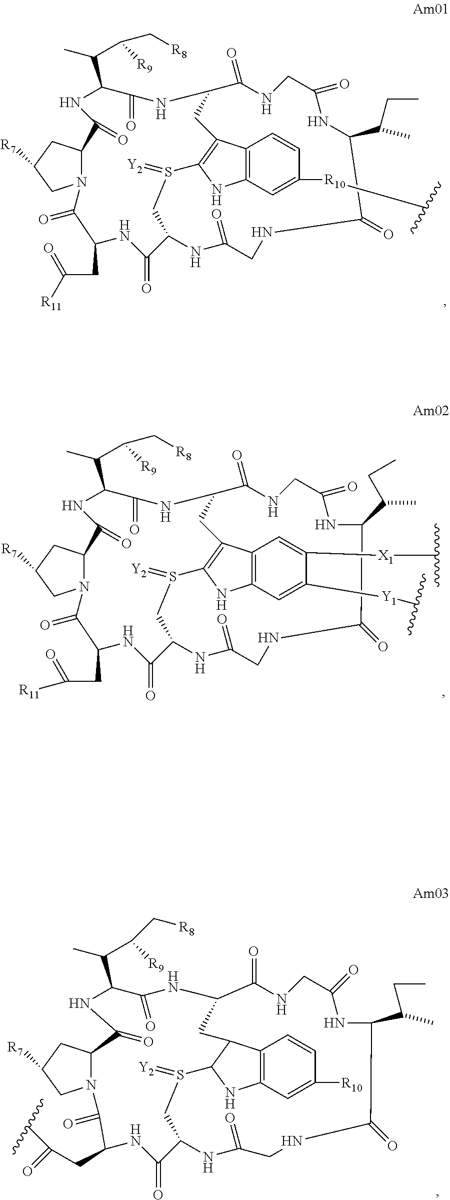

or an isotope of one or more chemical elements, or pharmaceutically acceptable salts, hydrates, or hydrated salts; or the polymorphic crystalline structures of these compounds; or the optical isomers, racemates, diastereomers or enantiomers; wherein X₁, and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) and C(O)NR₁; R₇, R₈, and R₉ are independently H, OH, OR₁, NH₂, NHR₁, C₁-C₆ alkyl, or absent; Y₂ is O, O₂, NR₁, NH, or absent; R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH-(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R₁′, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, or NH(CH₂CH₂O)ₚCH₂CH₂NHPO₃H₂, wherein (Aa)ᵣ is 1-8 aminoacids; n and mi are independently 1-20; p is 1-5000; R₁ and Ar, are the same defined in Formula (I).

Protein kinase inhibitors that block the action of an enzyme to add a phosphate (PO₄) group to serine, threonine, or tyrosine amino acids on a protein, and can modulate the protein function. The protein kinase inhibitors can be used to treat diseases due to hyperactive protein kinases (including mutant or overexpressed kinases) in cancer or to modulate cell functions to overcome other disease drivers. The structures of protein kinase inhibitors are preferred to selected from Adavosertib, Afatinib, Axitinib, Bafetinib, Bosutinib, Cobimetinib, Crizotinib, Cabozantinib, Dasatinib, Entrectinib, Erdafitinib, Erlotinib, Erlotinib, Fostamatinib, Gefitinib, Ibrutinib, Imatinib, Lapatinib, Lenvatinib, Mubritinib, Nilotinib, Pazopanib, Pegaptanib, Ponatinib, Rebastinib, Regorafenib, Ruxolitinib, Sorafenib, Sunitinib, SU6656, Tofacitinib, Vandetanib, and Vemurafenib, having the following formula, PK01˜PK29:

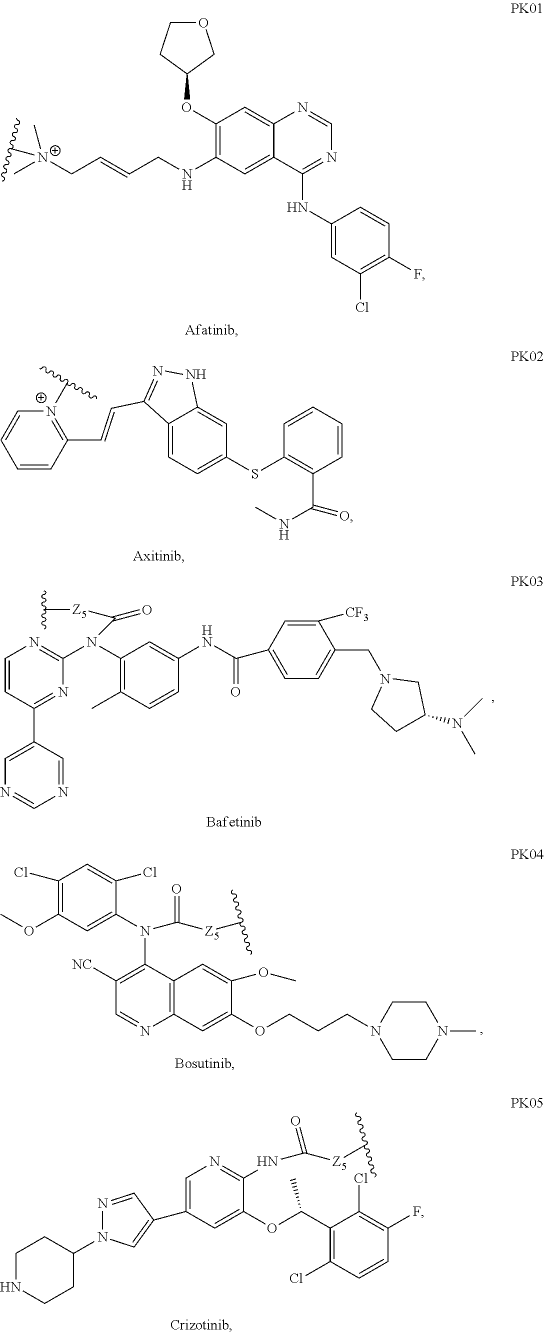

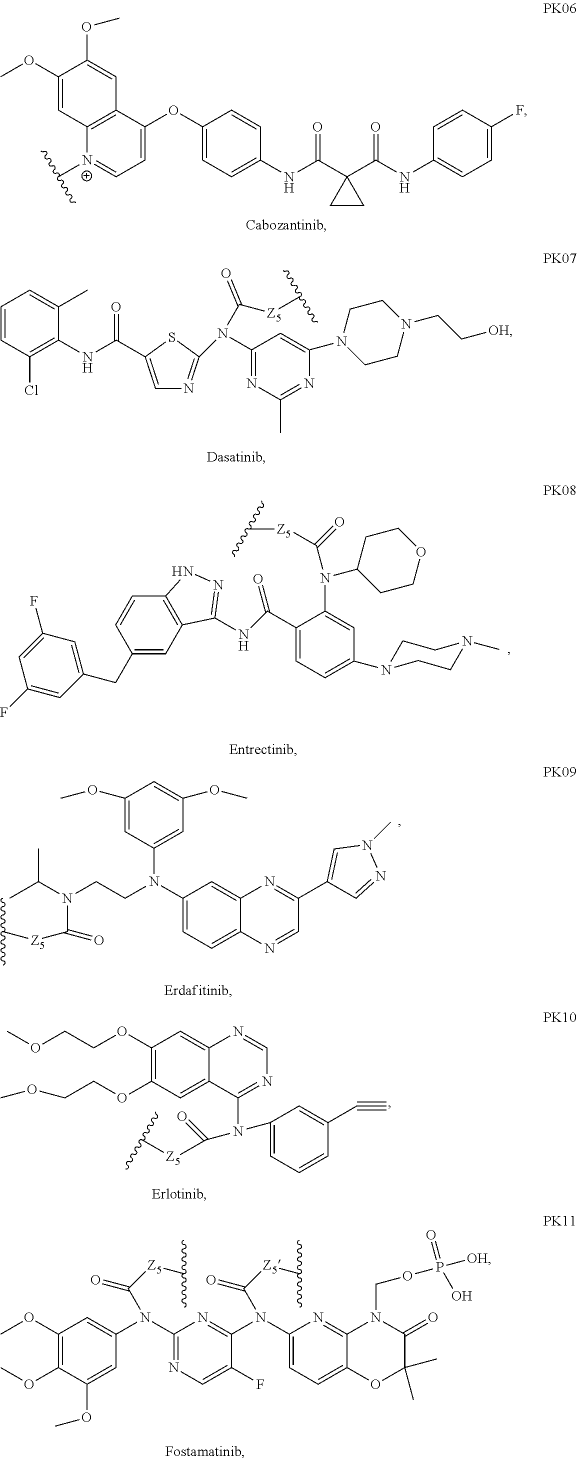

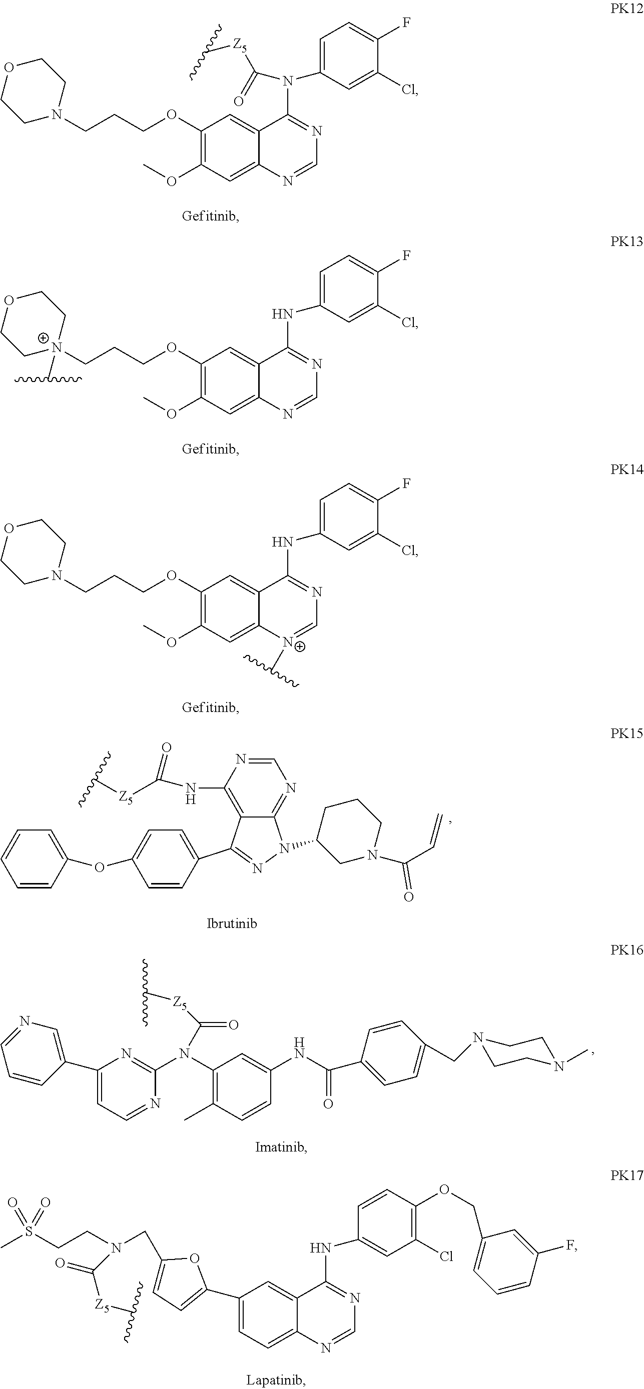

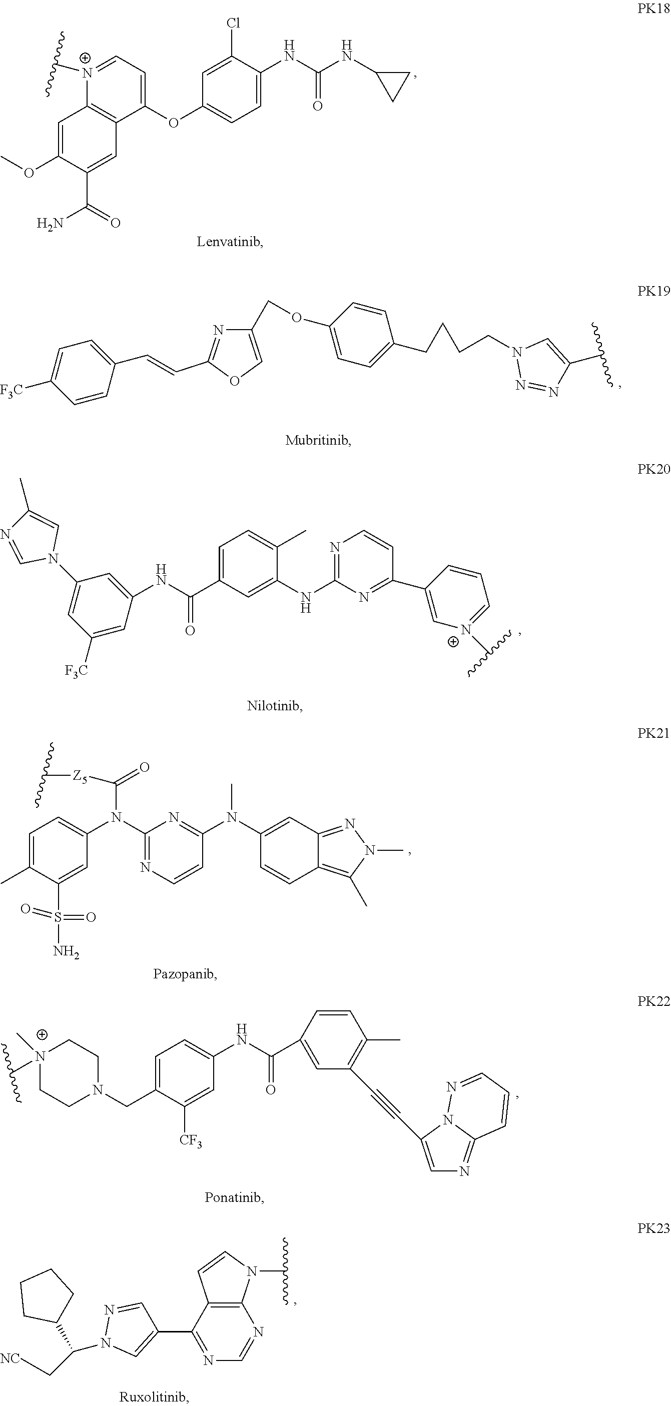

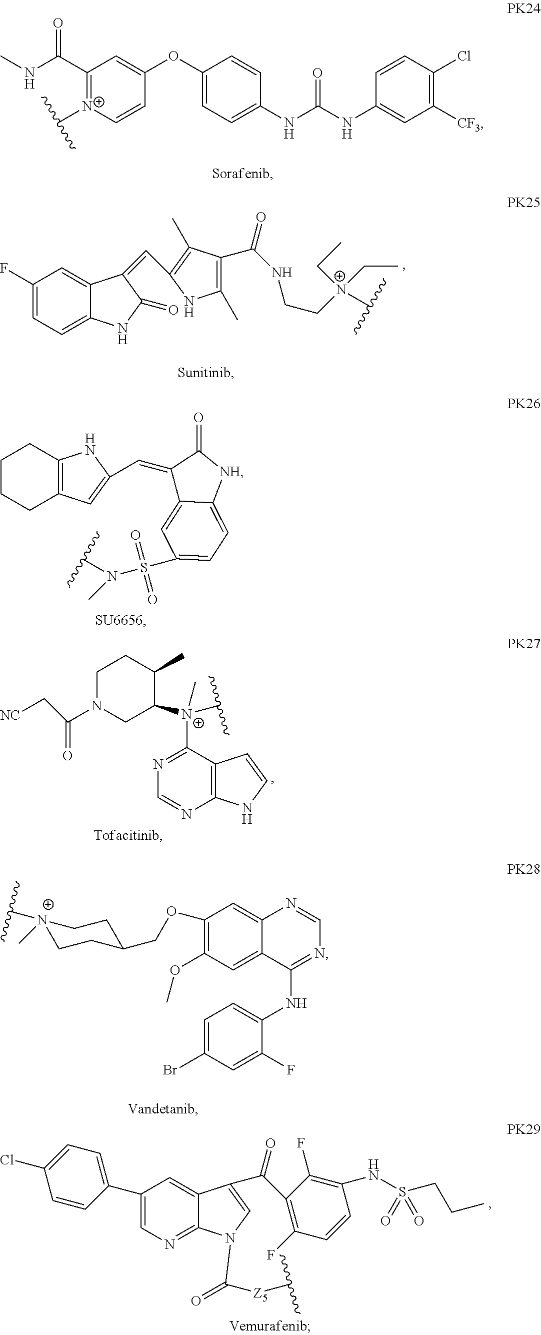

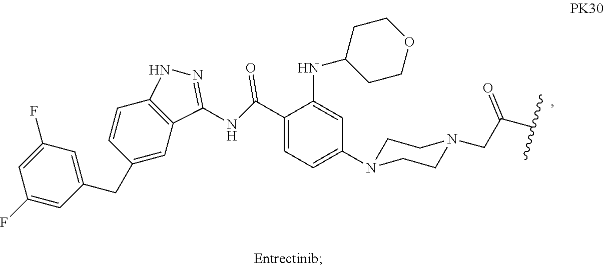 [SMILES: *C(=O)CN1CCN(c2ccc(C(=O)Nc3n[nH]c4ccc(Cc5cc(F)cc(F)c5)cc34)c(NC3CCOCC3)c2)CC1]

A MEK inhibitor inhibits the mitogen-activated protein kinases MEK1 and/or MEK2 which is often overactive in some cancers. MEK inhibitors are especially used for treatment of BRAF-mutated melanoma, and KRAS/BRAF mutated colorectal cancer, breast cancer, and non-small cell lung cancer (NSCLC). MEK inhibitors are selected from PD0325901, selumetinib (AZD6244), cobimetinib (XL518), refametinib, trametinib (GSK1120212), pimasertib, Binimetinib (MEK162), AZD8330, RO4987655, RO5126766, WX-554, E6201, GDC-0623, PD-325901 and TAK-733. The preferred MEK inhibitors are selected from Trametinib (GSK1120212), Cobimetinib (XL518), Binimetinib (MEK162), selumetinib having the following formula:

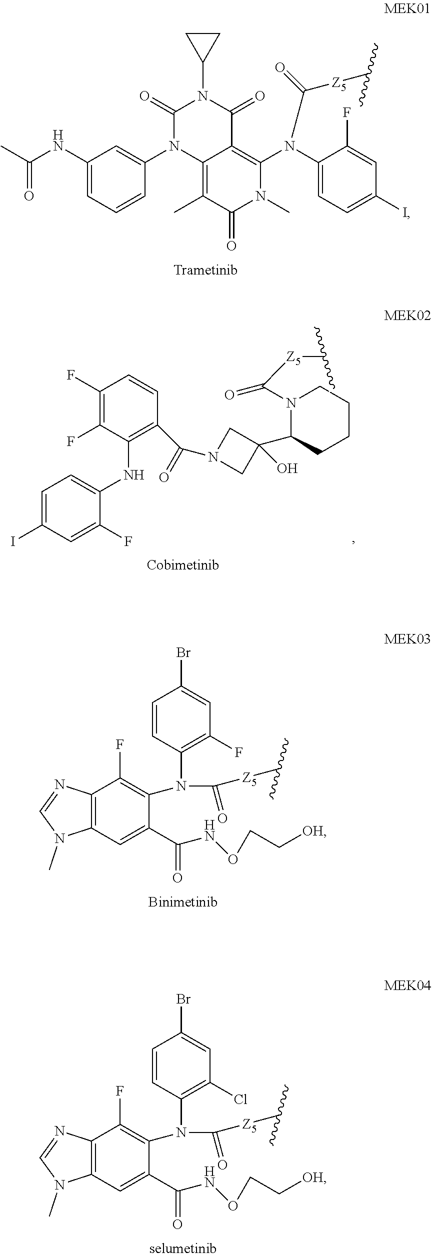

wherein Z₅ is selected from O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) and C(O)NR₁;

A proteinase inhibitor that are used as a payload is preferably selected from: Carfilzomib, Clindamycin, Retapamulin, Indibulin, as shown in the following structures:

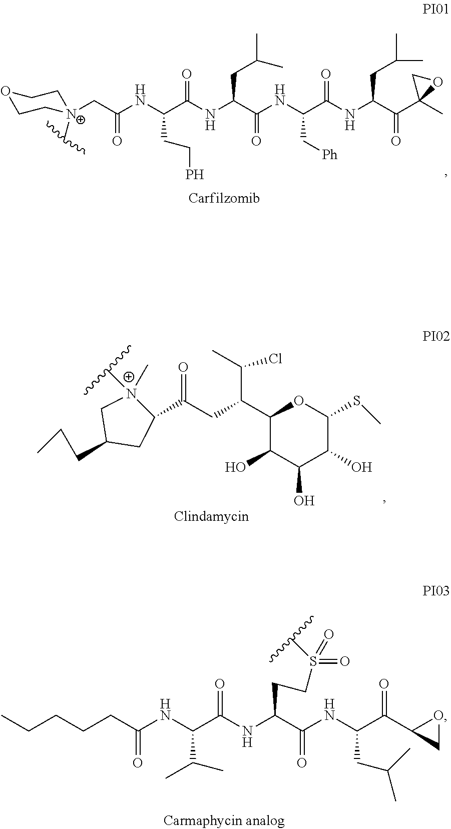

An immunotoxin herein is a macromolecular drug which is usually a cytotoxic protein derived from a bacterial or plant protein, such as Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, etc. It also can be a highly toxic bacterial pore-forming protoxin that requires proteolytic processing for activation. An example of this protoxin is proaerolysin and its genetically modified form, topsalysin. Topsalysin is a modified recombinant protein that has been engineered to be selectively activated by an enzyme in the prostate, leading to localized cell death and tissue disruption without damaging neighboring tissue and nerves; An immunotoxin herein is preferably conjugated via the side-chain linker of the application through an amino acid having free amino, thiol or carboxyl acid group; and more preferably through N-terminal amino acid.

Additionally W, L₁, L₂, V₁, and V₂, may independently be composed of one or more linker components of 6-maleimidocaproyl (“MC”), maleimidopropanoyl (“MP”), valine-citrulline (“val-cit” or “vc”), alanine-phenylalanine (“ala-phe” or “af”), p-aminobenzyloxy-carbonyl (“PAB”), 4-thiopentanoate (“SPP”), 4-(N-maleimidomethyl)cyclohexane-1 carboxylate (“MCC”), (4-acetyl)amino-benzoate (“SIAB”), 4-thio-butyrate (SPDB), 4-thio-2-hydroxysulfonyl-butyrate (2-Sulfo-SPDB), as the structures shown below or natural or unnatural peptides having 1˜12 natural or unnatural amino acid unites. The natural amino acid is preferably selected from aspartic acid, glutamic acid, arginine, histidine, lysine, serine, threonine, asparagine, glutamine, cysteine, selenocysteine, tyrosine, phenylalanine, glycine, proline, tryptophan, and alanine;

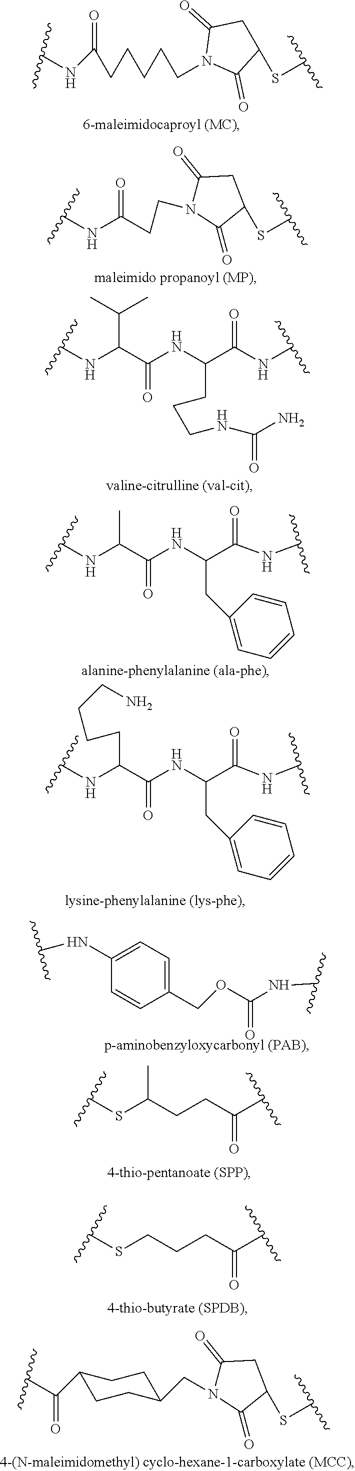

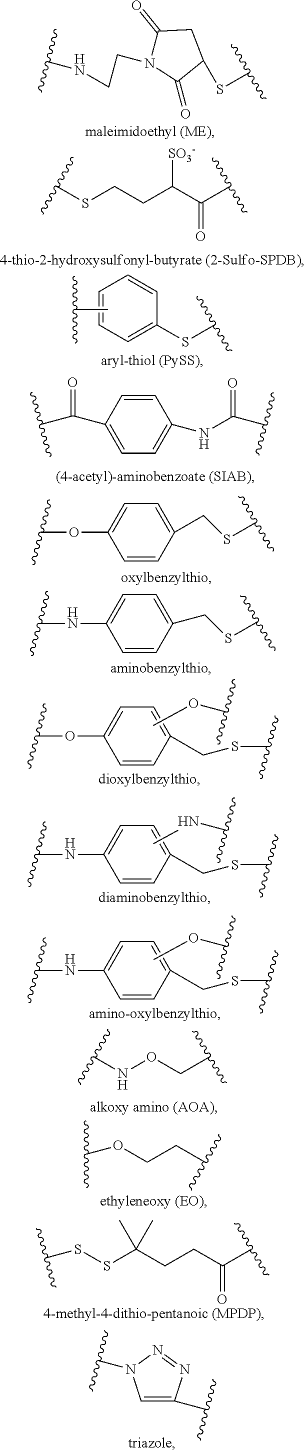

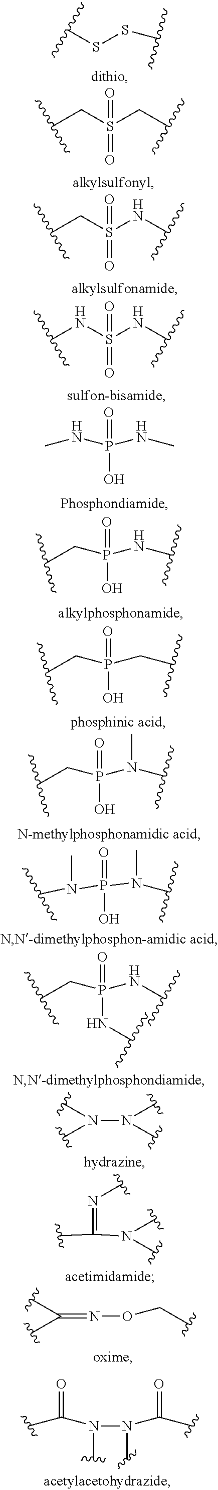

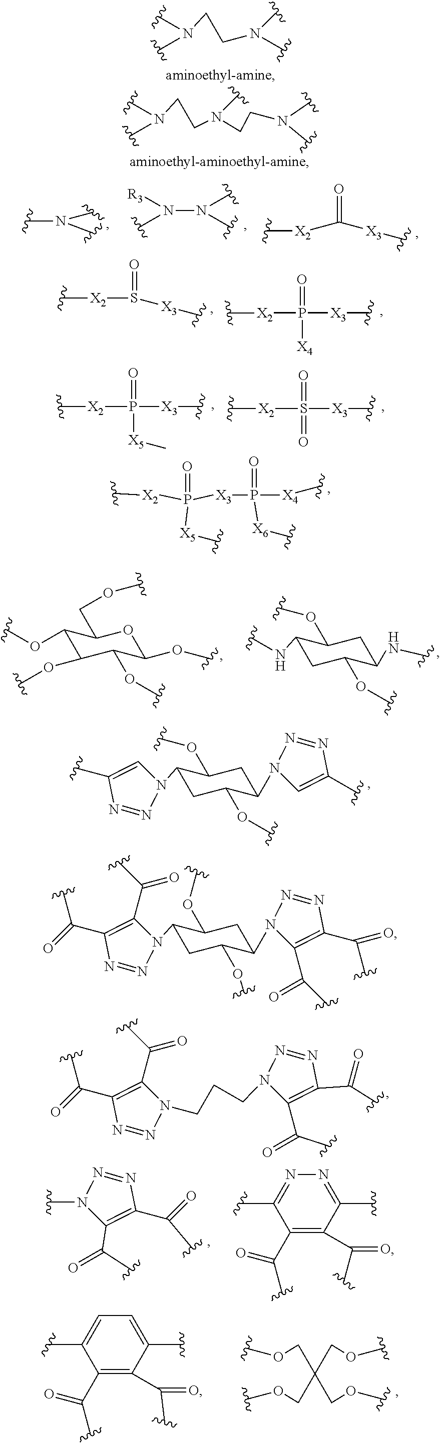

and L- or D-, natural or unnatural peptides containing 1-20 amino acids; wherein [symbol] is the site of linkage; Preferably X₂, X₃, X₄, X₅, or X₆, are independently selected from NH; NHNH; N(R₁₂); N(R₁₂)N(R_{12′}); O; S; C₁-C₆ of alkyl; C₂-C₆ of heteroalkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1˜8 amino acids; wherein R₁₂ and R_{12′} are independently H; C₁-C₈ of alkyl; C₂-C₈ of hetero-alkyl, alkylcycloalkyl, heterocycloalkyl; C₃-C₈ of aryl, Ar-alkyl, heterocyclic, carbocyclic, cycloalkyl, heteroalkylcycloalkyl, alkylcarbonyl, heteroaryl; or 1-8 carbon atoms of esters, ether, or amide; or polyethyleneoxy unit of formula (OCH₂CH₂)ₚ or (OCH₂CH(CH₃))ₚ, wherein p is an integer from 0 to about 1000, or combination above thereof;

W, L₁, L₂ V₁, and V₂ may also independently contain a self-immolative or a non-self-immolative component, peptidic units, a hydrazone bond, a disulfide, an ester, an oxime, an amide, or a thioether bond. The self-immolative unit includes, but is not limited to, aromatic compounds that are electronically similar to the para-aminobenzylcarbamoyl (PAB) groups such as 2-aminoimidazol-5-methanol derivatives, heterocyclic PAB analogs, beta-glucuronide, and ortho or para-aminobenzylacetals;

Preferably, the self-immolative linker component has one of the following structures:

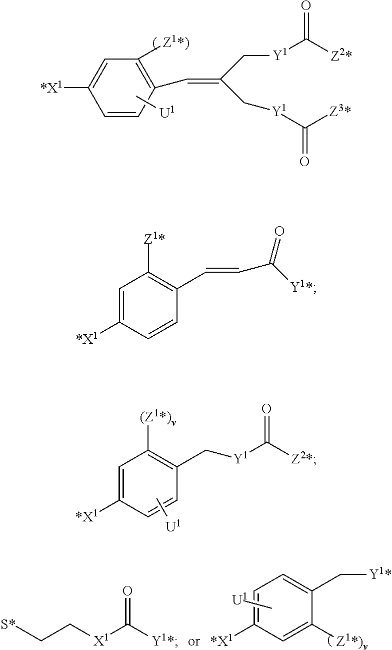

wherein the (*) atom is the point of attachment of additional spacer or releasable linker units, or the cytotoxic agent, and/or the binding molecule (CBA); X¹, Y¹, Z² and Z³ are independently NH, O, or S; Z¹ is independently H, NHR₁, OR₁, SR₁, COX₁R₁, wherein X₁ and R₁ are defined above; v is 0 or 1; U¹ is independently H, OH, C₁˜C₆ alkyl, (OCH₂CH₂)ₙ, F, Cl, Br, I, OR⁵, SR⁵, NR⁵R⁵′, N═NR₅, N═R₅, NR⁵R^{5′}, NO₂, SOR⁵R^{5′}, SO₂R⁵, SO₃R⁵, OSO₃R⁵, PR⁵R^{5′}, POR⁵R^{5′}, PO₂R⁵R^{5′}, OPO(OR⁵)(OR^{5′}), or OCH₂PO(OR⁵(OR^{5′}), wherein R⁵ and R^{5′} are independently selected from H, C₁˜C₈ of alkyl; C₂˜C₈ of alkenyl, alkynyl, heteroalkyl, or amino acid; C₃˜C₈ of aryl, heterocyclic, carbocyclic, cycloalkyl, heterocycloalkyl, heteroaralkyl, alkylcarbonyl, or glycoside; or pharmaceutical cation salts;

W, L₁, L₂ V₁, and V₂ may also independently contain non-self-immolative linker component having one of the following structures:

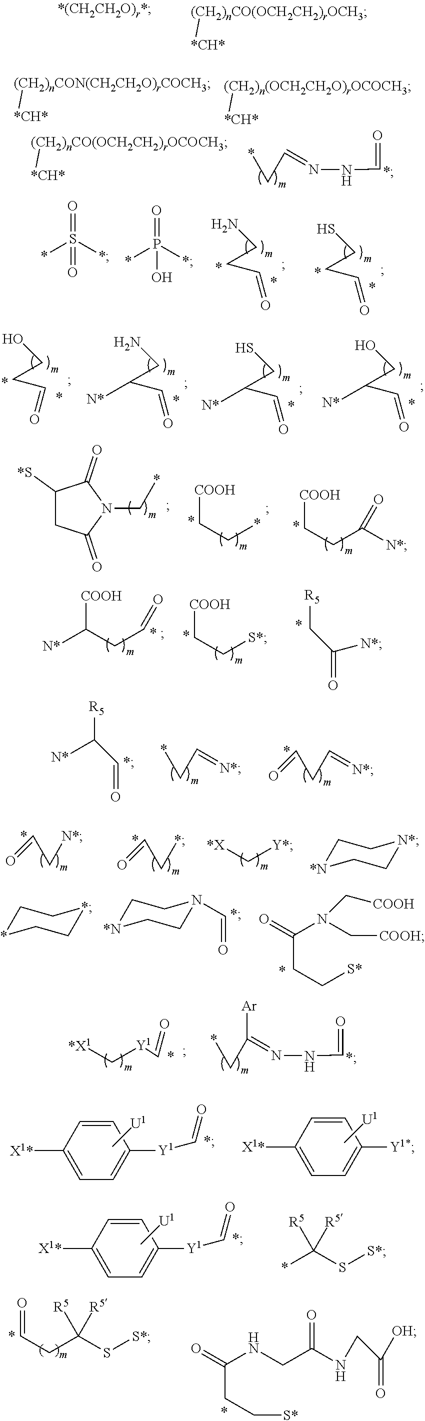

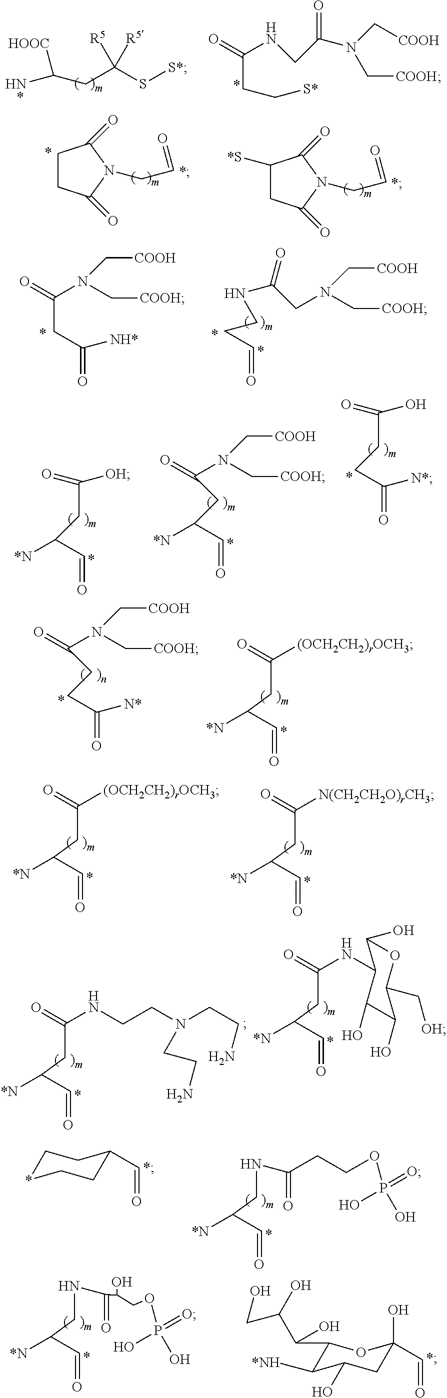

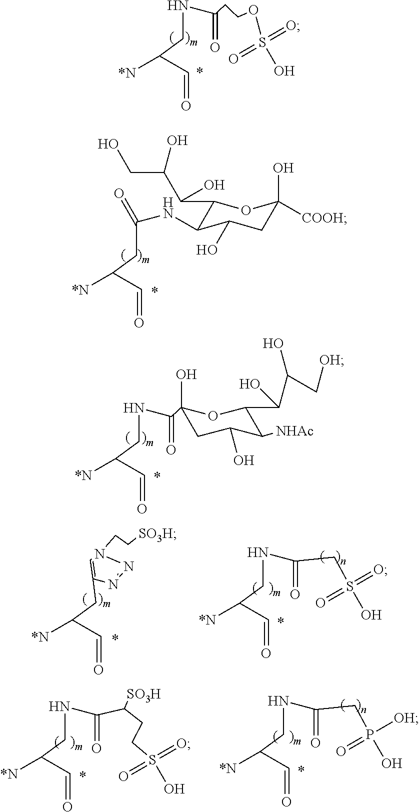

wherein the (*) atom is the point of attachment of additional spacer or releasable linkers, the cytotoxic agents, and/or the binding molecules; X¹, Y¹, U¹, R⁵, R^{5′} are defined as above; r is 0˜100; m and n are 0˜20 independently; Further preferably, W, L₁, L₂ V₁, and V₂ may independently be a releasable linker component. The term releasable refers to a linker that includes at least one bond that can be broken under physiological conditions, such as a pH-labile, acid-labile, base-labile, oxidatively labile, metabolically labile, biochemically labile or enzyme-labile bond. It is appreciated that such physiological conditions resulting in bond breaking do not necessarily include a biological or metabolic process, and instead may include a standard chemical reaction, such as a hydrolysis or substitution reaction, for example, an endosome having a lower pH than cytosolic pH, and/or disulfide bond exchange reaction with a intracellular thiol, such as a millimolar range of abundant of glutathione inside the malignant cells;

Examples of the releasable components of W, L₁, L₂, V₁, and V₂ independently include, but not limited: —(CR₁₅R₁₆)ₘ(Aar)r(CR₁₇R₁₈)ₙ(OCH₂CH₂)ₜ—, —(CR₁₅R₁₆)ₘ(CR₁₇R₁₈)ₙ(Aa)ᵣ(OCH₂CH₂)ₜ—, - (Aa)ᵣ-(CR₁₅R₁₆)ₘ(CR₁₇R₁₈)ₙ(OCH₂CH₂)ₜ—, —(CR₁₅R₁₆)ₘ(CR₁₇R₁₈)ₙ(OCH₂CH₂)ᵣ(Aa)ₜ-, —(CR₁₅R₁₆)ₘ(CR₁₇═CR₁₈)(CR₁₉R₂₀)ₙ(Aa)ₜ(OCH₂CH₂)ᵣ—, —(CR₁₅R₁₆)ₘ(NR₁₁CO)(Aa)ₜ(CR₁₉R₂₀)ₙ—(OCH₂CH₂)ᵣ—, —(CR₁₅R₁₆)ₘ(Aa)ₜ(NR₂₁CO)(CR₁₉R₂₀)ₙ(OCH₂CH₂)ᵣ—, —(CR₁₅R₁₆)ₘ(OCO)(Aa)ₜ-(CR₁₉R₂₀)ₙ(OCH₂CH₂)ᵣ—, —(CR₁₅R₁₆)ₘ(OCNR₁₇)(Aa)ₜ(CR₁₉R₂₀)ₙ(OCH₂CH₂)ᵣ—, —(CR₁₅R₁₆)ₘ—(CO)(Aa)ₜ-(CR₁₉R₂₀)ₙ(OCH₂CH₂)ᵣ—, —(CR₁₅R₁₆)ₘ(NR₂₁CO)(Aa)ₜ(CR₁₉R₂₀)ₙ(OCH₂CH₂)ₜ—, —(CR₁₅R₁₆)ₘ—(OCO)(Aa)ₜ(CR₁₉R₂₀)ₙ—(OCH₂CH₂)ᵣ—, —(CR₁₅R₁₆)ₘ(OCNR₁₇)(Aa)ₜ(CR₁₉R₂₀)ₙ—(OCH₂CH₂)ᵣ—, —(CR₁₅R₁₆)ₘ(CO)(Aa)ₜ(CR₁₉R₂₀)ₙ—(OCH₂CH₂)ᵣ—, —(CR₁₅R₁₆)ₘ-phenyl-CO(Aa)ₜ-(CR₁₇R₁₈)ₙ—, —(CR₁₅R₁₆)ₘ-furyl-CO(Aa)ₜ(CR₁₇R₁₈)ₙ—, —(CR₁₅R₁₆)ₘ-oxazolyl-CO(Aa)ₜ(CR₁₇R₁₈)ₙ—, —(CR₁₅R₁₆)ₘ-thiazolyl-CO(Aa)ₜ(CCR₁₇R₁₈)ₙ—, —(CR₁₅R₁₆)ₜ-thienyl-CO(CR₁₇R₁₈)ₙ—, —(CR₁₅R₁₆)ₜ-imidazolyl-CO—(CR₁₇R₁₈)ₙ—, —(CR₁₅R₁₆)ₜ-morpholino-CO(Aa)ₜ-(CR₁₇R₁₈)ₙ—, —(CR₁₅R₁₆)ₜ-piperazino-CO(Aa)ₜ(CR₁₇R₁₈)ₙ—, —(CR₁₅R₁₆)ₜ—N-methylpiperazin-CO(Aa)ₜ(CR₁₇R₁₈)ₙ—, —(CR₁₅R₁₆)ₘ-(Aa)ₜphenyl-, —(CR₁₅R₁₆)ₘ-(Aa)ₜfuryl-, —(CR₁₅R₁₆)ₘ-oxazolyl(Aa)ₜ-, —(CR₁₅R₁₆)ₘ-thiazolyl(Aa)ₜ-, —(CR₁₅R₁₆)ₘ-thienyl-(Aa)ₜ-, —(CR₁₅R₁₆)ₘ-imidazolyl(Aa)ₜ-, —(CR₁₅R₁₆)ₘ-morpholino-(Aa)ₜ-, —(C₁₅R₁₆)ₘ-piperazino-(Aa)ₜ-, —(CR₁₅R₁₆)ₘ—N-methylpiperazino-(Aa)ₜ-,

—K(CR₁₅R₁₆)ₘ(Aa)r(CR₁₇R₁₈)ₙ(OCH₂CH₂)ₜ—, —K(CR₁₅R₁₆)ₘ(CR₁₇R₁₈)ₙ(Aa)ᵣ(OCH₂CH₂)ₜ—, —K(Aa)ᵣ-(CR₁₅R₁₆)ₘ(CR₁₇R₁₈)ₙ(OCH₂CH₂)ₜ—, —K(CR₁₅R₁₆)ₘ(CR₁₇R₁₈)ₙ(OCH₂CH₂)ᵣ(Aa)ₜ-, —K(CR₁₅R₁₆)ₘ(CR₁₇═CR₁₈)(CR₁₉R₂₀)ₙ(Aa)ₜ(OCH₂CH₂)ᵣ, —K(CR₁₅R₁₆)ₘ(NR₁₁CO)(Aa)ₜ-(CR₁₉R₂₀)ₙ(OCH₂CH₂)ᵣ—, —K(CR₁₅R₁₆)ₘ(Aa)ₜ(NR₂₁CO)(CR₁₉R₂₀)ₙ(OCH₂CH₂)ᵣ—, —K(CR₁₅R₁₆)ₘ(O—CO)(Aa)ₜ(CR₁₉R₂₀)ₙ(OCH₂CH₂)ᵣ—, —K(CR₁₅R₁₆)ₘ(OCNR₁₇)(Aa)ₜ(CR₁₉R₂₀)ₙ(OCH₂CH₂)ᵣ—, —K(CR₁₅R₁₆)ₘ(CO)(Aa)ₜ-(CR₁₉R₂₀)ₙ(OCH₂CH₂)ᵣ—, —K(CR₁₅R₁₆)ₘ(NR₂₁CO)(Aa)ₜ(CR₁₉R₂₀)ₙ—(OCH₂CH₂)ᵣ—, —K(CR₁₅R₁₆)ₘ—(OCO)(Aa)ₜ(CR₁₉R₂₀)ₙ(OCH₂CH₂)ᵣ—, —K(CR₁₅R₁₆)ₘ(OCNR₁₇)(Aa)ₜ-(CR₁₉R₂₀)ₙ((OCH₂CH₂)ᵣ—, —K—(CR₁₅R₁₆)ₘ(CO)(Aa)ₜ(CR₁₉R₂₀)ₙ(OCH₂CH₂)ᵣ—, —K(CR₁₅R₁₆)ₘ-phenyl-CO(Aa)ₜ(CR₁₇R₁₈)ₙ—, —K—(CR₁₅R₁₆)ₘ-furyl-CO(Aa)ₜ(CR₁₇R₁₈)ₙ—, —K(CR₁₅R₁₆)ₘ-oxazolyl-CO(Aa)ₜ(CR₁₇R₁₈)ₙ—, —K(CR₁₅R₁₆)ₘ-thiazolyl-CO(Aa)ₜ-(CR₁₇R₁₈)ₘ—, —K(CR₁₅R₁₆)ₜ-thienyl-CO(CR₁₇R₁₈)ₙ—, —K(CR₁₅R₁₆)ₜimidazolyl-CO—(CR₁₇R₁₈)ₙ—, —K(CR₁₅R₁₆)ₜmorpholino-CO(Aa)ₜ-(CR₁₇R₁₈)ₙ—, —K(CR₁₅R₁₆)ₜ-piperazino-CO(Aa)ₜ-(CR₁₇R₁₈)ₘ—, —K(CR₁₅R₁₆)ₜ—N-methylpiperazin-CO(Aa)ₜ(CR₁₇R₁₈)ₙ—, —K(CR₁₅R₁₆)ₘ-(Aa)ₜphenyl, —K—(CR₁₅R₁₆)ₘ-(Aa)ₜfuryl-, —K(CR₁₅R₁₆)ₘ-oxazolyl-(Aa)ₜ-, —K(CR₁₅R₁₆)ₘ-thiazolyl(Aa)ₜ-, —K(CR₁₅R₁₆)ₘ-thienyl-(Aa)ₜ-, —K(CR₁₅R₁₆)ₘ-imidazolyl(Aa)ₜ-, —K(C₁₅R₁₆)ₘ-morpholino(Aa)ₜ-, —K(CR₁₅R₁₆)ₘpiperazino(Aa)ₜG, —K(CR₁₅R₁₆)ₘ

—N-methylpiperazino(Aa)ₜ-; wherein m, Aa, n, R₁₃, R₁₄, and R₁₅ are described above; t and r here are 0-100 independently; R₁₆, R₁₇, R₁₈, R₁₉, and R₂₀ are independently chosen from H; halide; C₁˜C₈ of alkyl or heteroalkkyl, C₂˜C₈ of aryl, alkenyl, alkynyl, ether, ester, amine or amide, C₃˜C₈ of aryl, which optionally substituted by one or more halide, CN, NR₁₂R_{12′}, CF₃, OR₁₂, Aryl, heterocycle, S(O)R₁₂, SO₂R₁₂, —CO₂H, —SO₃H, —OR₁₂, —CO₂R₁₂, —CONR₁₂, —PO₂R₁₂R₁₃, —PO₃H or P(O)R₁₂R_{12′}R₁₃; K is NR₁₂, —SS—, —C(═O)—, —C(═O)NH—, —C(═O)O—, —C═NH—O—, —C═N—NH—, —C(═O)NH—NH—, O, S, Se, B, Het (heterocyclic or heteroaromatic ring having C₃-C₁₂); or peptides containing the same or different 1-20 amino acids More preferably, components of W, L₁, L₂ V₁, and V₂ are independently linear alkyl having from 1-6 carbon atoms, or polyethyleneoxy unit of formula (OCH₂CH₂)ₚ, p=1˜5000, or a peptide containing 1˜4 units of amino acids (L or D form), or combination above.

Alternatively, any one or more of W, Q₁, Q₂, L₁, L₂, V₁, or V₂, can be independently absent but Q₁, and Q₂ are not absent at the same time.

Generally stated, in another aspect, when V₁ and/or V₂ linked to the cell-binding molecule, T, or when L₁ and/or L₂ directly linked to T (wherein V₁, and V₂, are absent), the conjugation linkage could have one or more of the following structures:

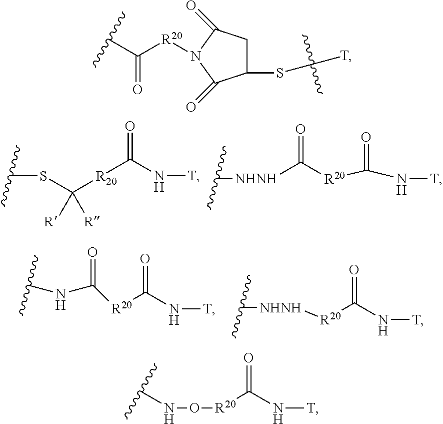

wherein R²⁰ and R²¹ are independently C₁˜C₈ alkyl; C₂˜C₈ heteroalkyl, or heterocyclic; C₃˜C₈ aryl, Ar-alkyl, cycloalkyl, alkylcycloalkyl, heterocycloalkyl, heteroalkylcycloalkyl, carbocyclic, or alkylcarbonyl; or C₂-C₁₀₀ polyethylene glycol having formula of (CH₂CH₂O)ₚ, p is defined above; or absent.

In another further aspect, Q₁ and Q₂ are preferably selected from a polyglycine or a polyalkylene glycol containing a C₂-C₁₈ lipid, or a C₂-C₁₈ fatty acid, or a C₂-C₁₈ fatty ammonium lipid. The polyalkylene glycol chain not only helps the conjugate more hydrophilic during the production, but also prevents the conjugate linker from hydrolysis by a hydrolase, e.g. a proteinase or an esterase. The lipid can help the conjugate to bind to an albumin in mammal bloods and then leads to the conjugate slowly dissociation from this complex during the blood circulation. Thus the side chain linker of the present patent application makes the conjugate more stable in the circulation. Polyalkylene glycols here include, but are not limited to, poly(ethylene glycols) (PEGs), poly(propylene glycol) and copolymers of ethylene oxide and propylene oxide; particularly preferred are PEGs, and more particularly preferred are monofunctionally activated hydroxyPEGs (e.g., hydroxyl PEGs activated at a single terminus, including reactive esters of hydroxyPEG-monocarboxylic acids, hydroxyPEG-monoaldehydes, hydroxyPEG-monoamines, hydroxyPEG-monohydrazides, hydroxyPEG-monocarbazates, hydroxyl PEG-monoiodo-acetamides, hydroxyl PEG-monomaleimides, hydroxyl PEG-monoorthopyridyl disulfides, hydroxyPEG-monooximes, hydroxyPEG-monophenyl carbonates, hydroxyl PEG-monophenyl glyoxals, hydroxyl PEG-monothiazolidine-2-thiones, hydroxyl PEG-monothioesters, hydroxyl PEG-monothiols, hydroxyl PEG-monotriazines and hydroxyl PEG-monovinylsulfones). The polyalkylene glycol has a molecular weight of from about 10 Daltons to about 200 kDa, preferably about 88 Da to about 40 kDa; two branch chains each with a molecular weight of about 88 Da to about 40 kDa; and more preferably two branches, each of about 88 Da to about 20 kDa. In one particular embodiment, the polyalkylene glycol is poly(ethylene) glycol and has a molecular weight of about 10 kDa; about 20 kDa, or about 40 kDa. In specific embodiments, the PEG is a PEG 10 kDa (linear or branched), a PEG 20 kDa (linear or branched), or a PEG 40 kDa (linear or branched). A number of US patents have disclosed the preparation of linear or branched “non-antigenic” PEG polymers and derivatives or conjugates thereof, e.g., U.S. Pat. Nos. 5,428,128; 5,621,039; 5,622,986; 5,643,575; 5,728,560; 5,730,990; 5,738,846; 5,811,076; 5,824,701; 5,840,900; 5,880,131; 5,900,402; 5,902,588; 5,919,455; 5,951,974; 5,965,119; 5,965,566; 5,969,040; 5,981,709; 6,011,042; 6,042,822; 6,113,906; 6,127,355; 6,132,713; 6,177,087, and 6,180,095.

A cell-binding agent/molecule, T, can be any kind presently known, or that become known, of a molecule that binds to, complexes with, or reacts with a moiety of a cell population sought to be therapeutically or otherwise biologically modified. Preferably the cell-binding agent/molecule is an immunotherapeutic protein, an antibody, a single chain antibody; an antibody fragment that binds to the target cell; a monoclonal antibody; a single chain monoclonal antibody; or a monoclonal antibody fragment that binds the target cell; a chimeric antibody; a chimeric antibody fragment that binds to the target cell; a domain antibody; a domain antibody fragment that binds to the target cell; adnectins that mimic antibodies; DARPins; a lymphokine; a hormone; a vitamin; a growth factor; a colony stimulating factor; or a nutrient-transport molecule (a transferrin); a binding peptides having over four amino acids, or protein, or antibody, or small cell-binding molecule or ligand attached on albumin, polymers, dendrimers, liposomes, nanoparticles, vesicles, or (viral) capsids;

Examples of Formula (I), (II), and (III) are illustrated below:

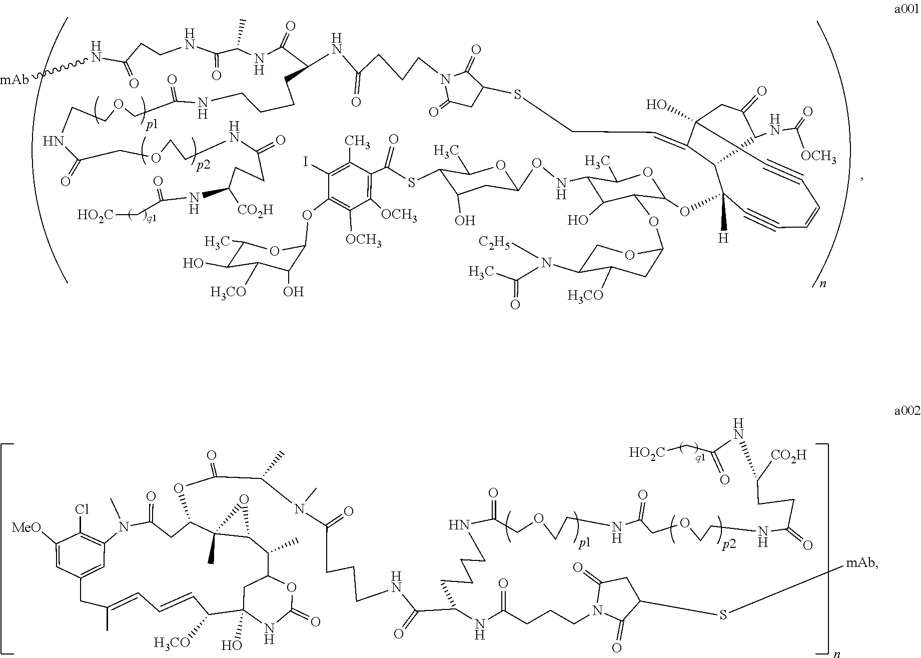

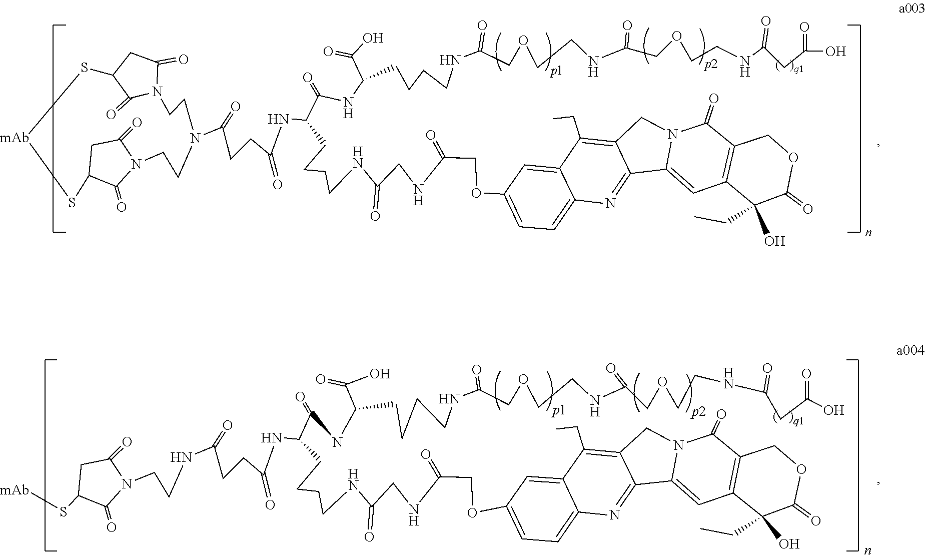

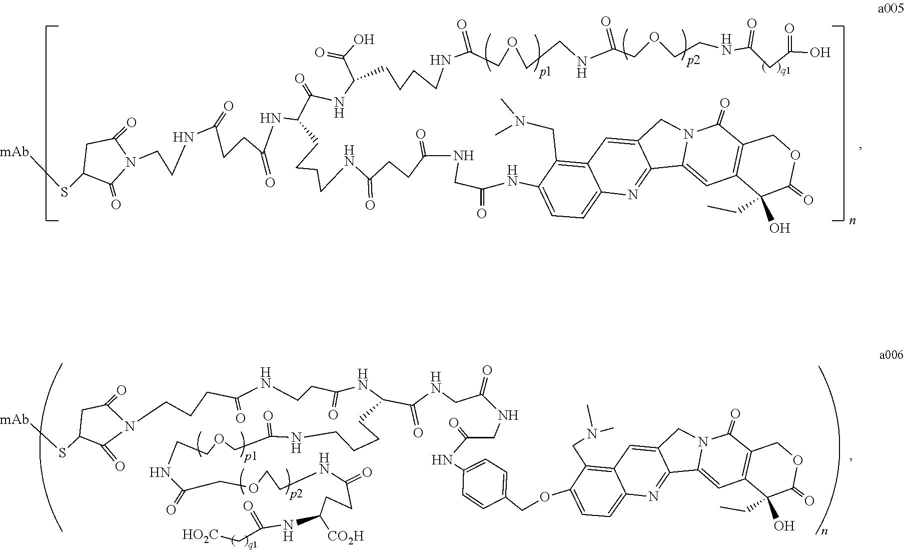

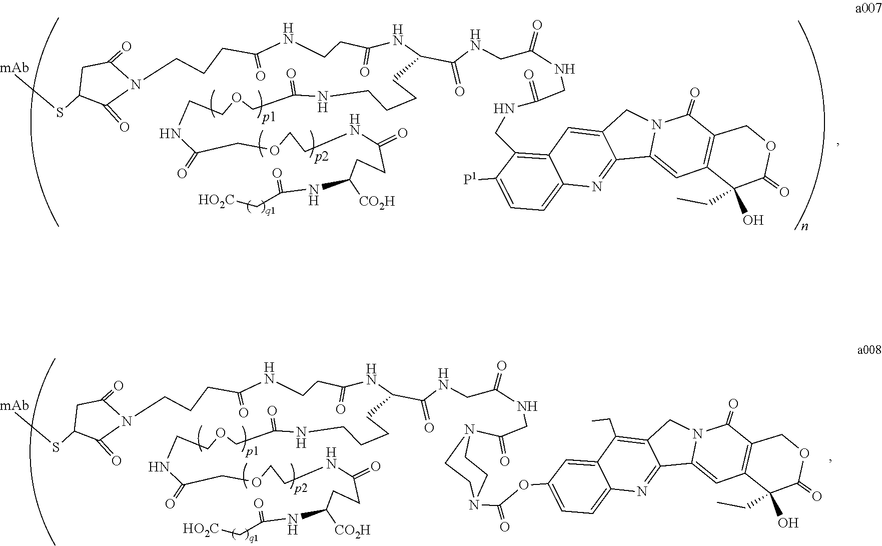

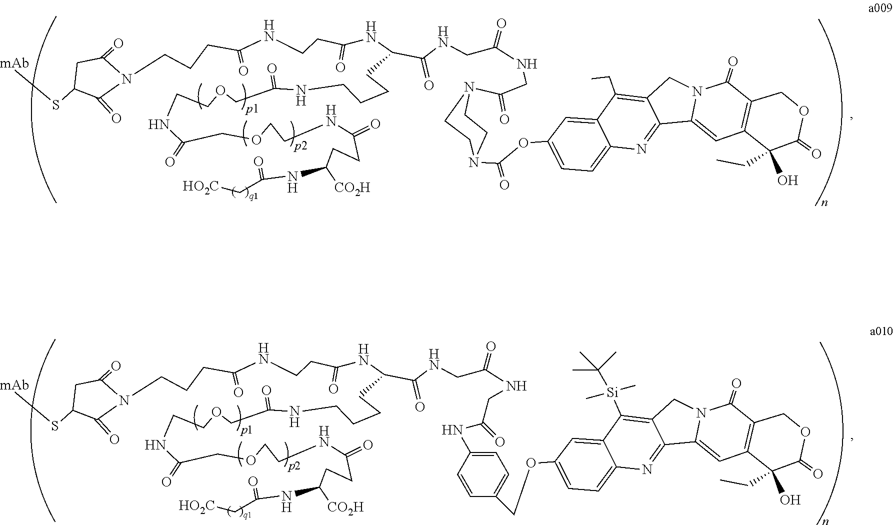

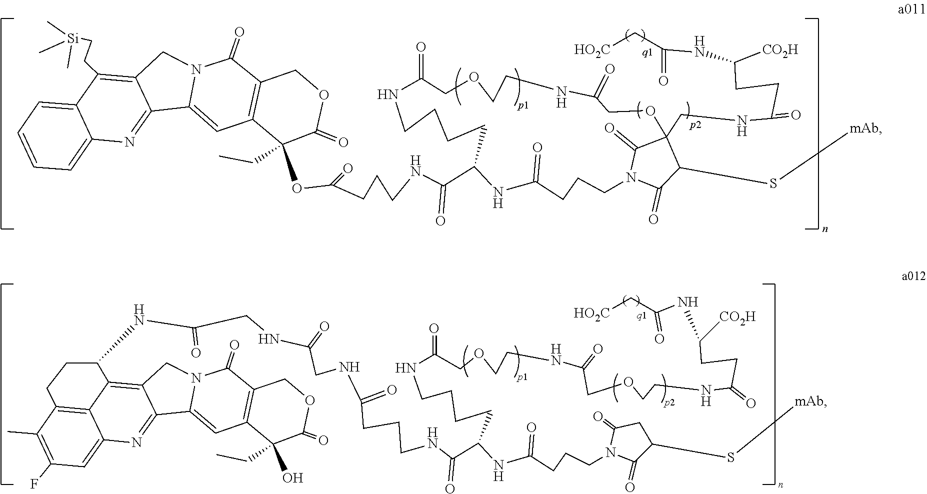

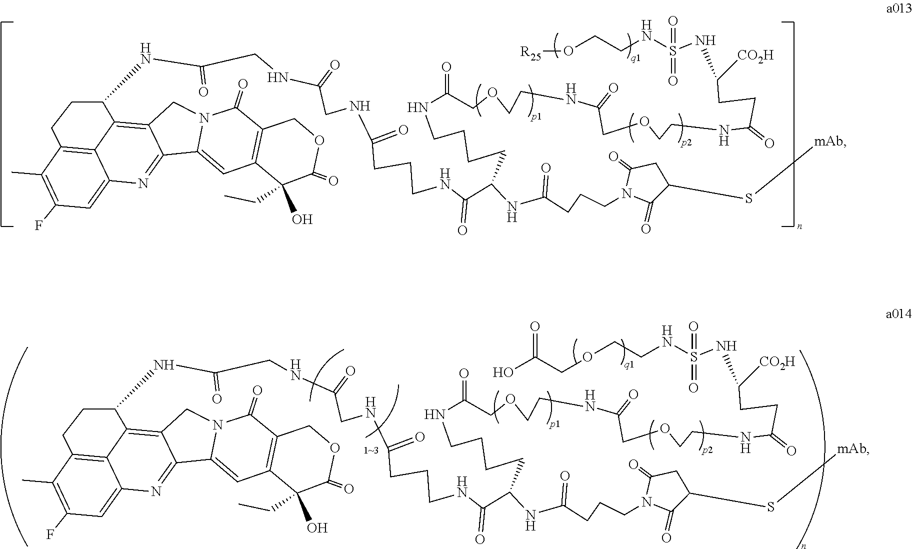

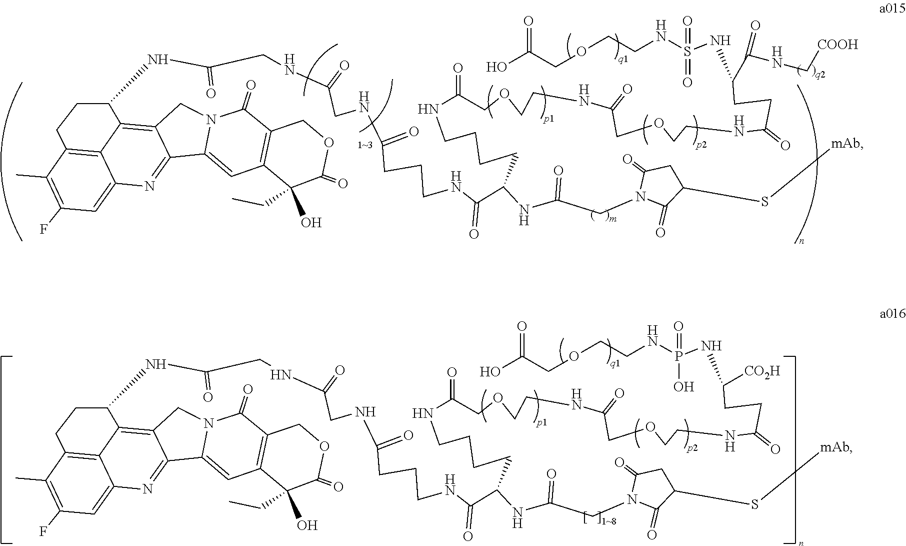

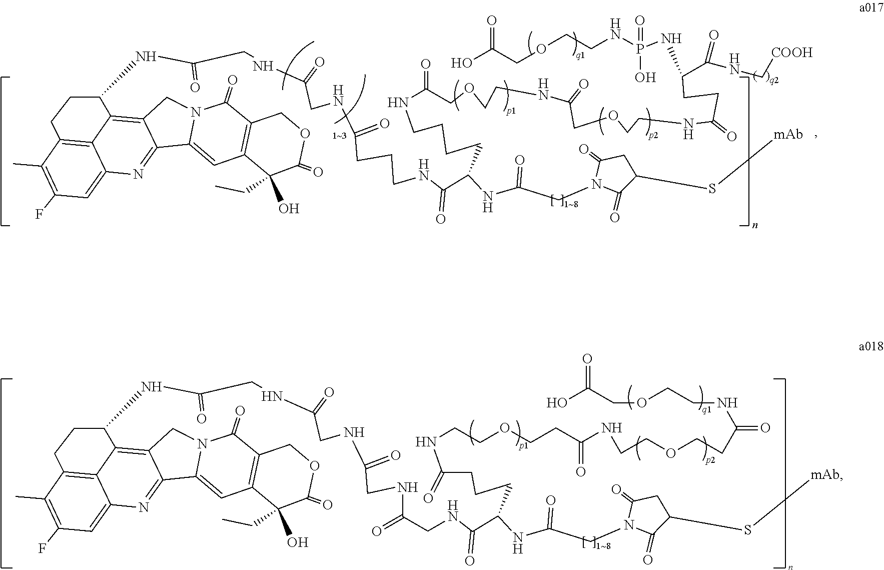

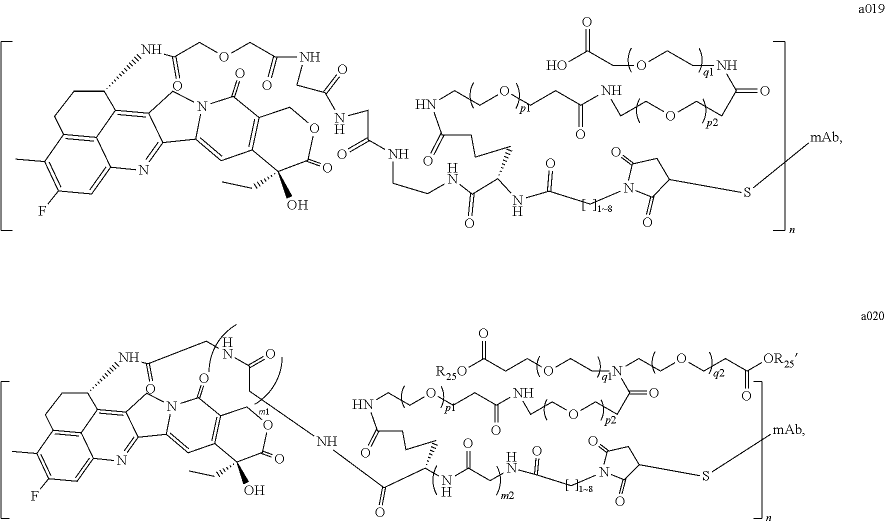

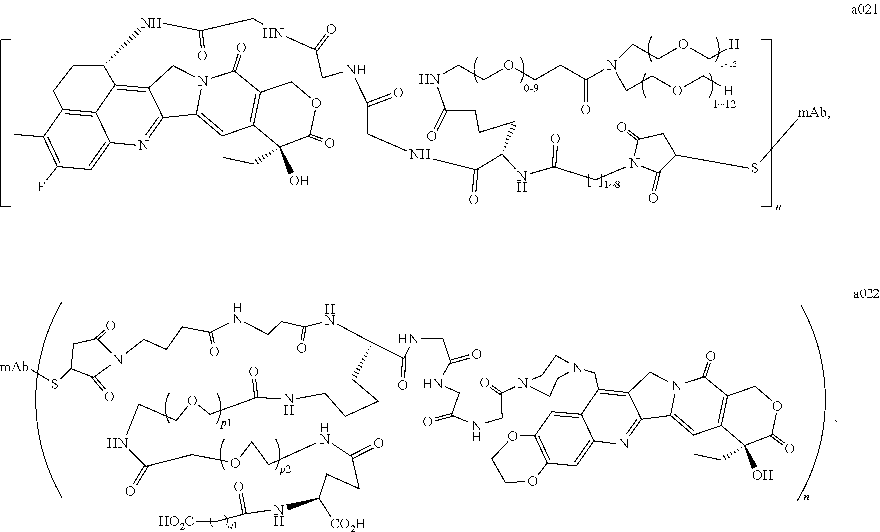

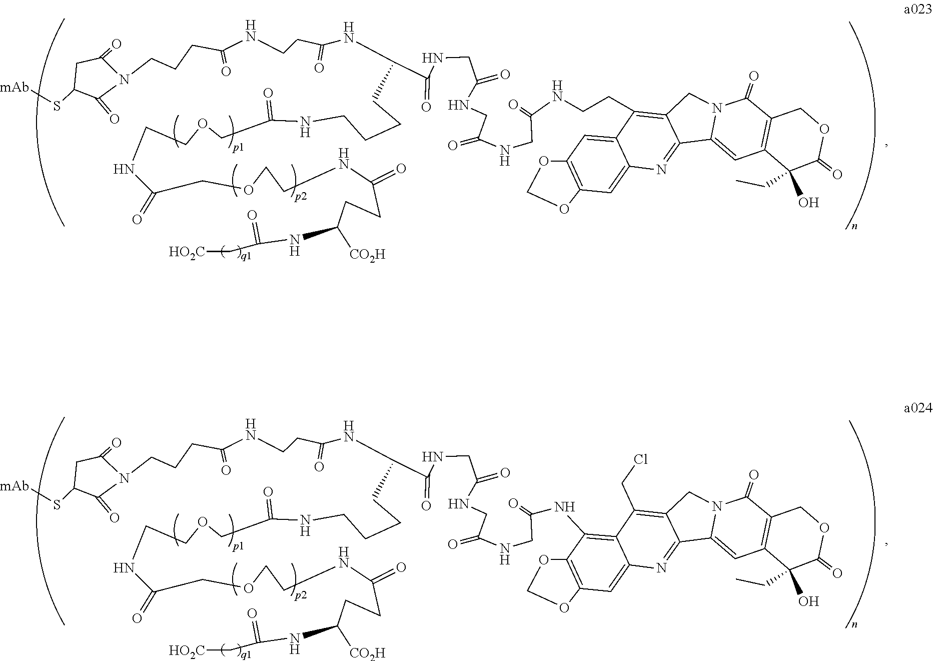

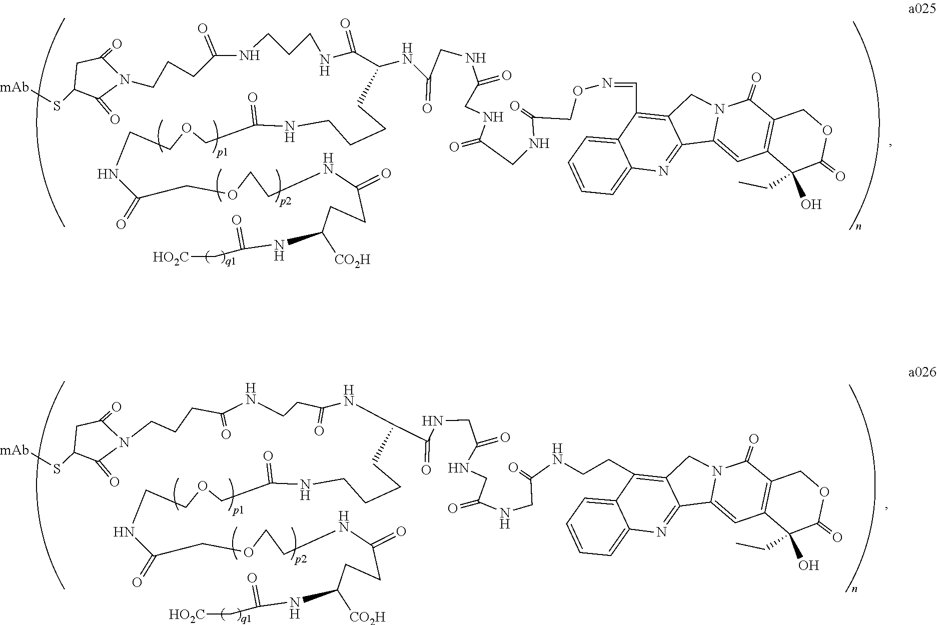

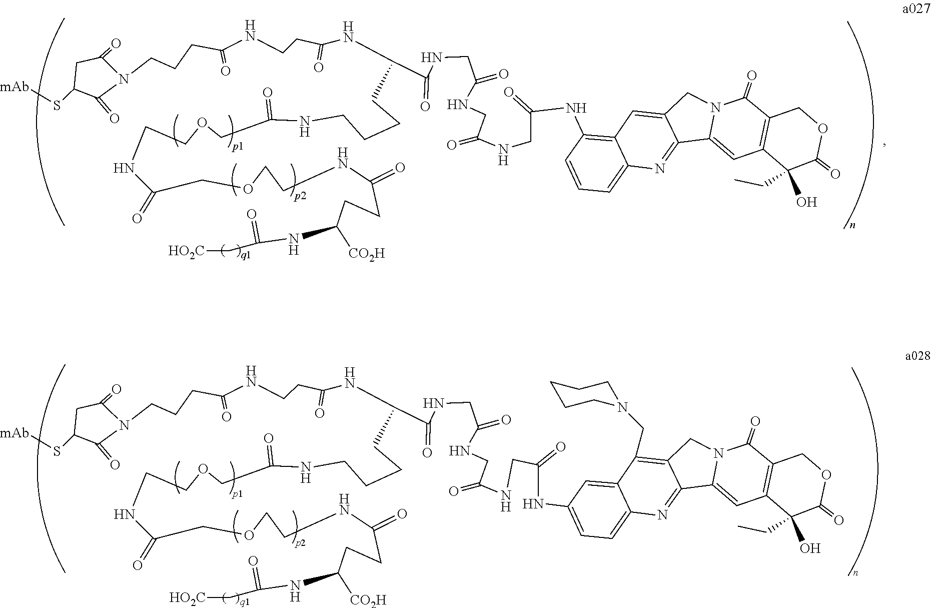

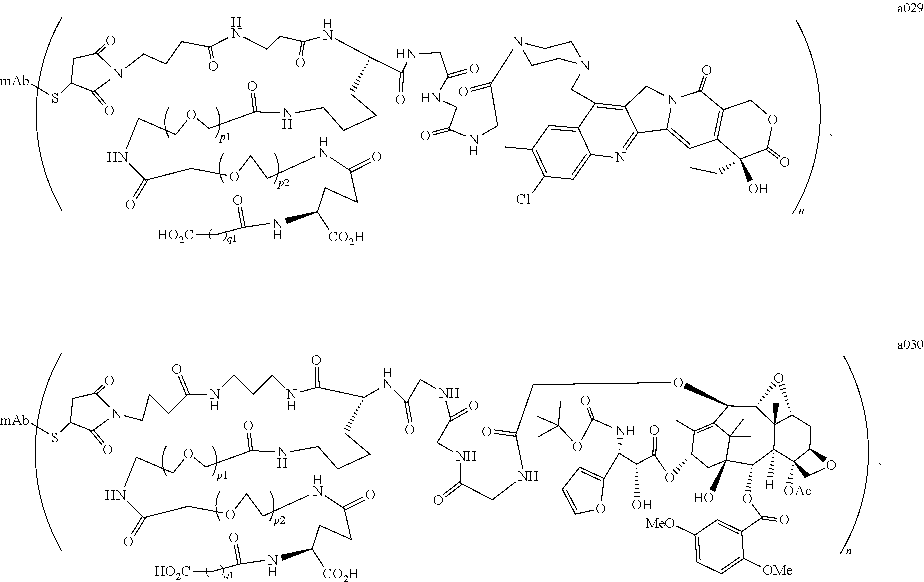

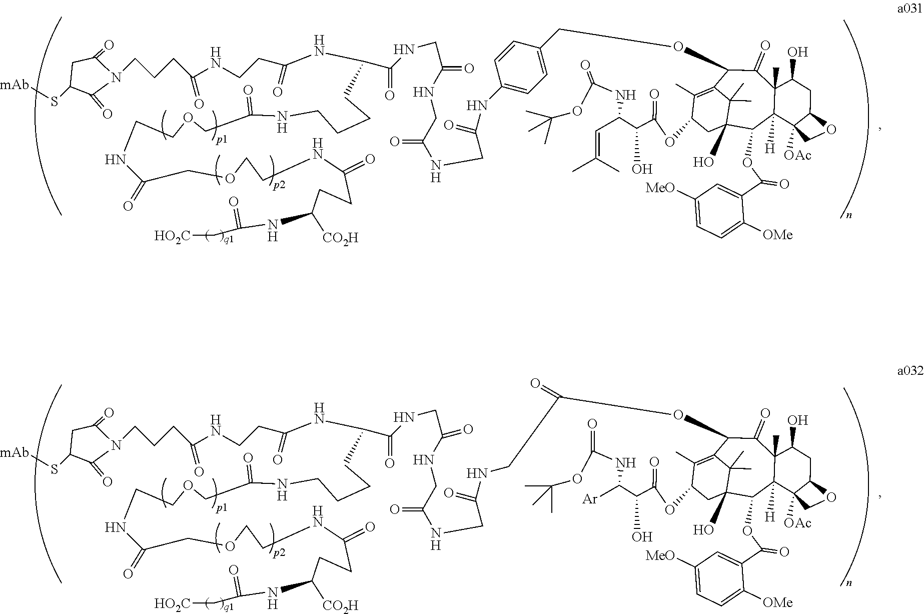

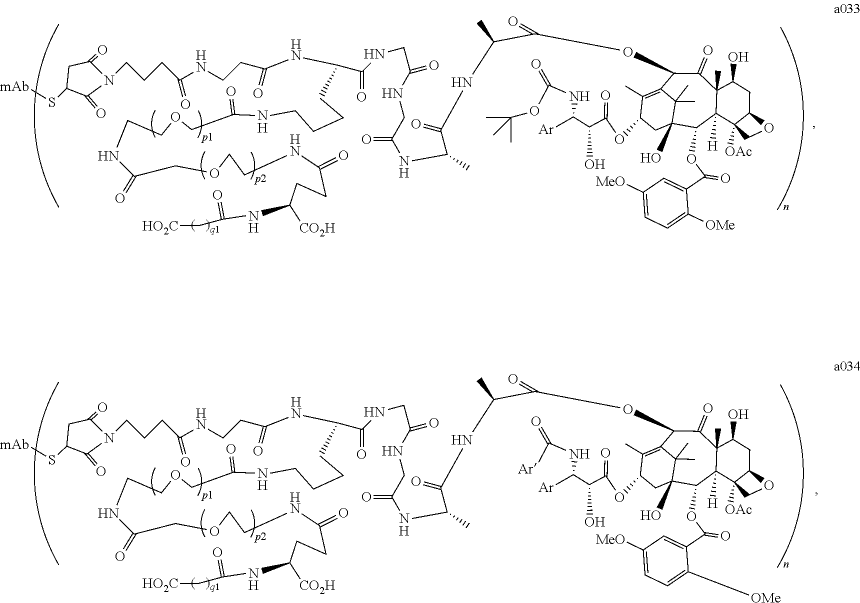

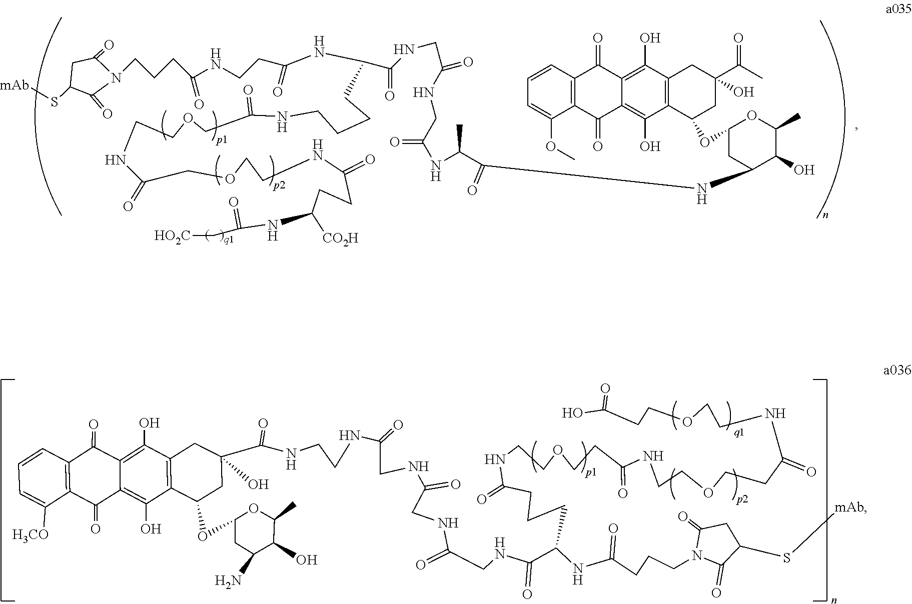

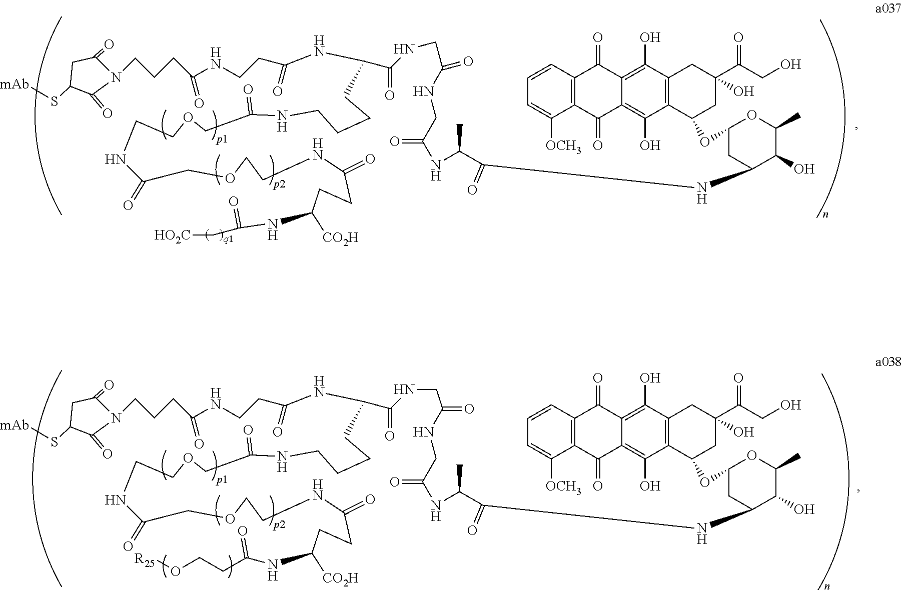

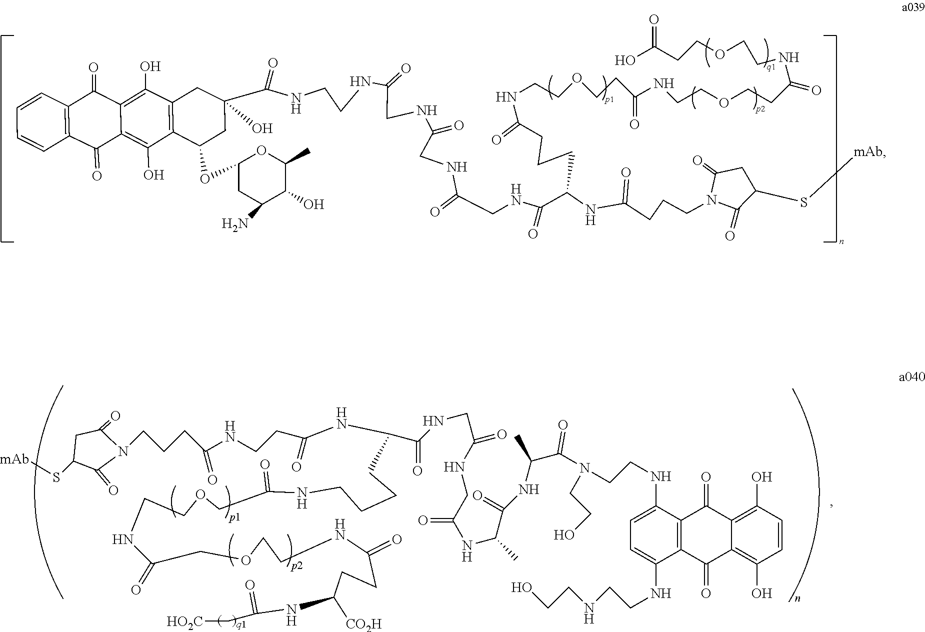

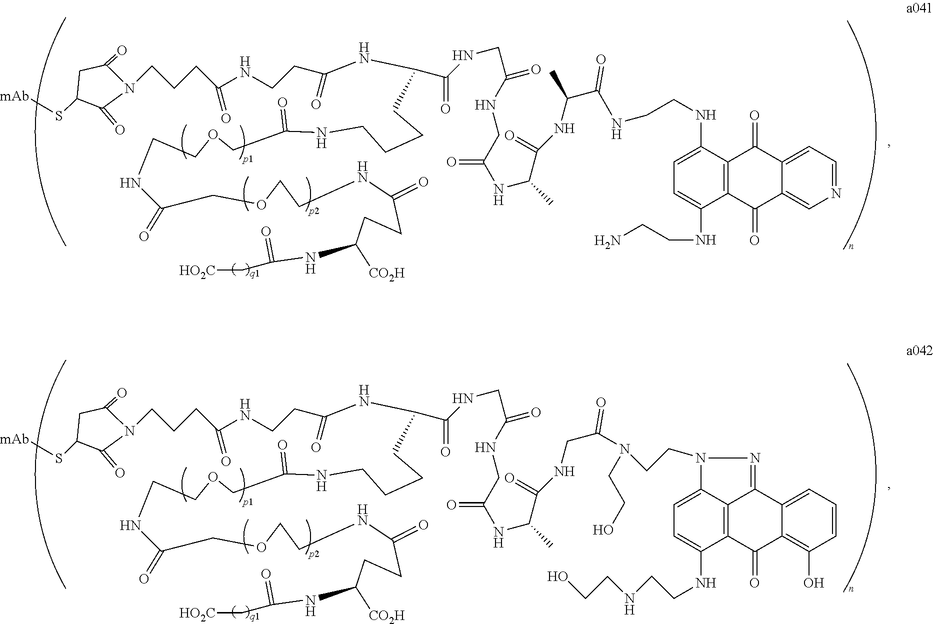

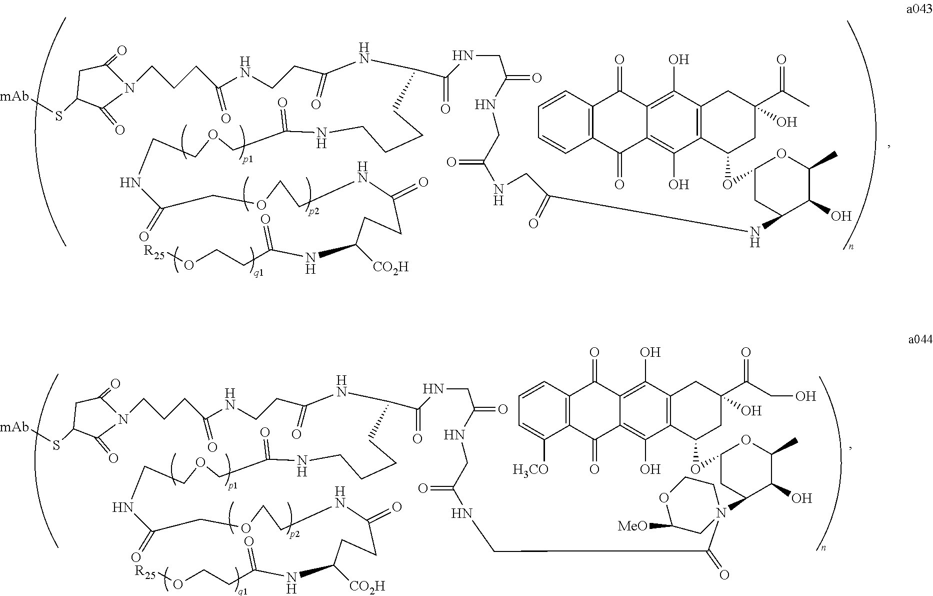

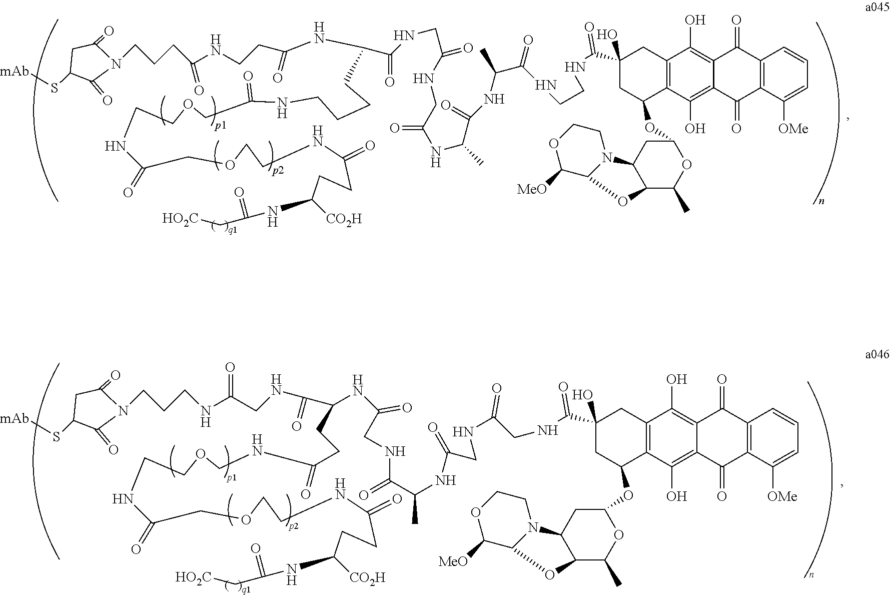

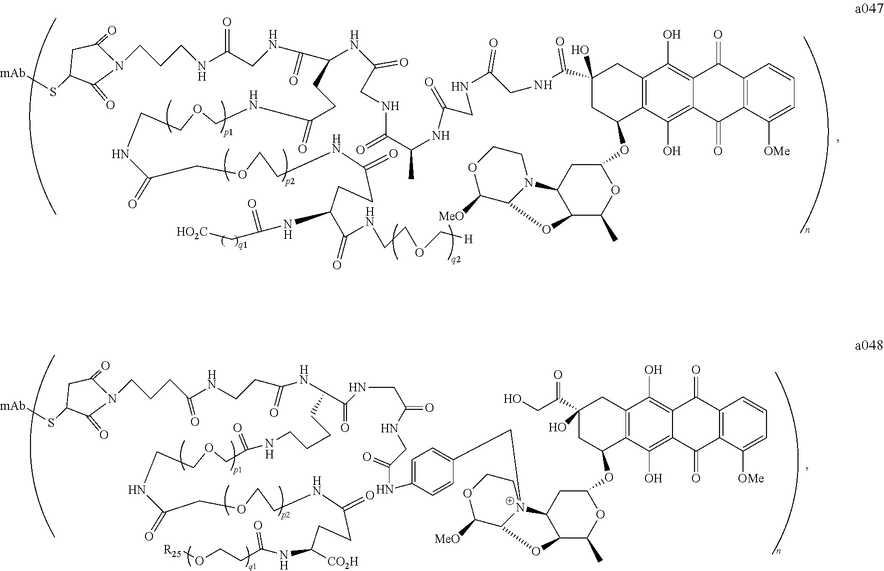

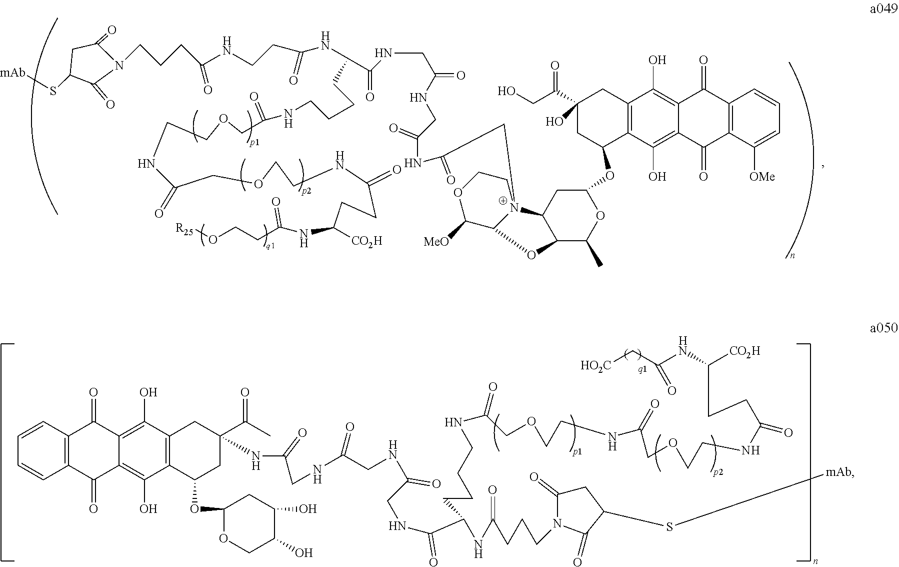

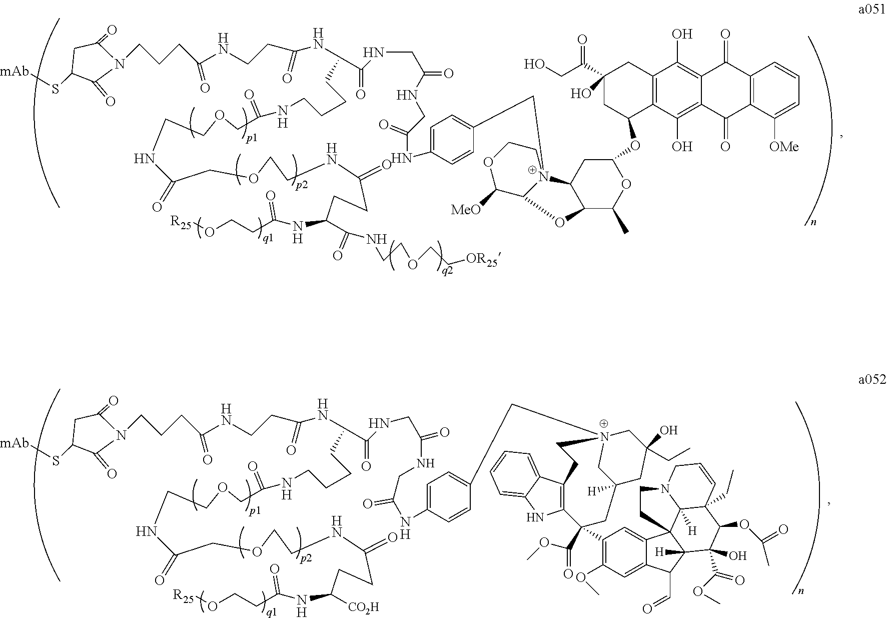

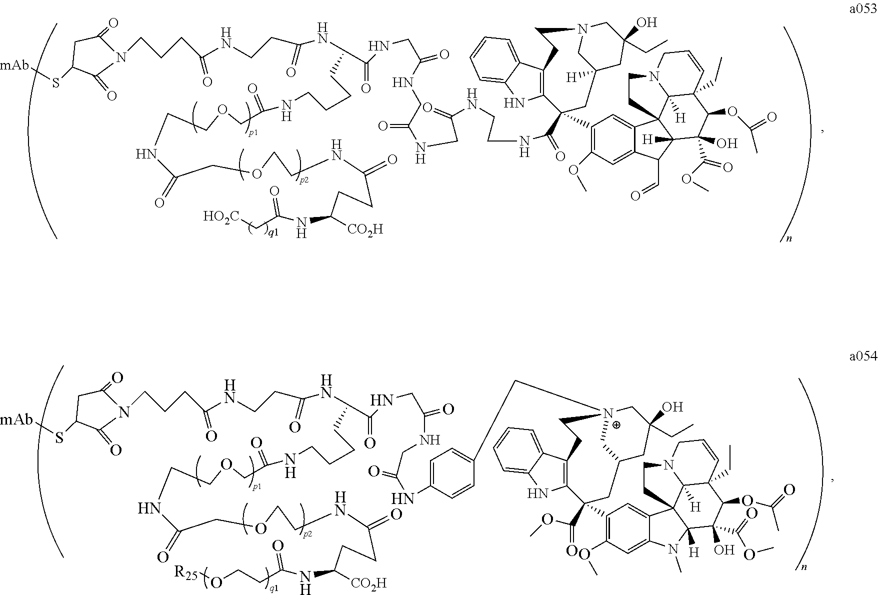

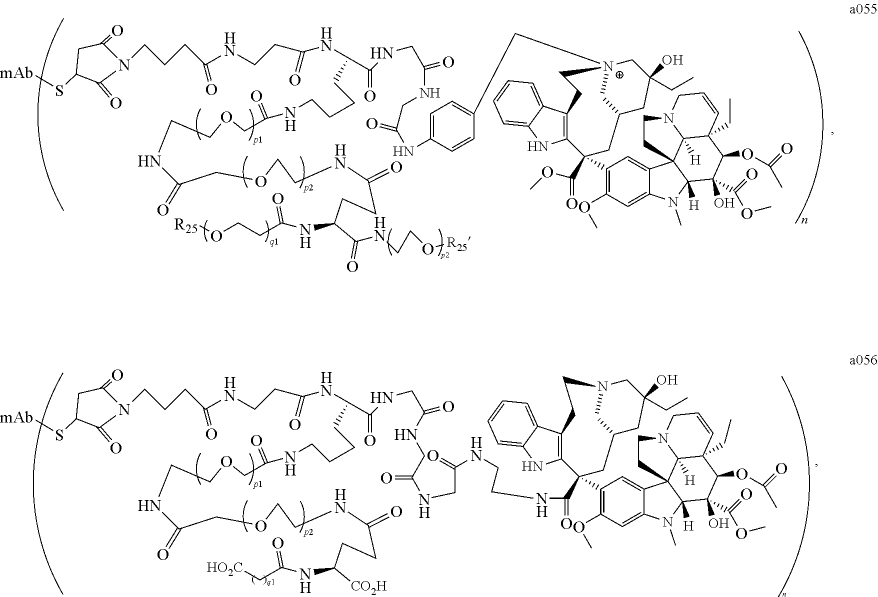

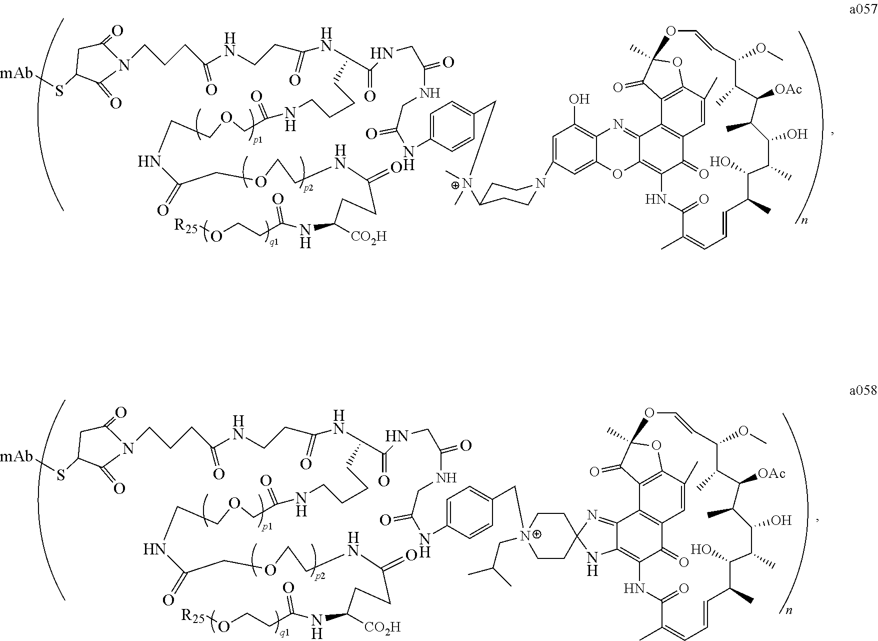

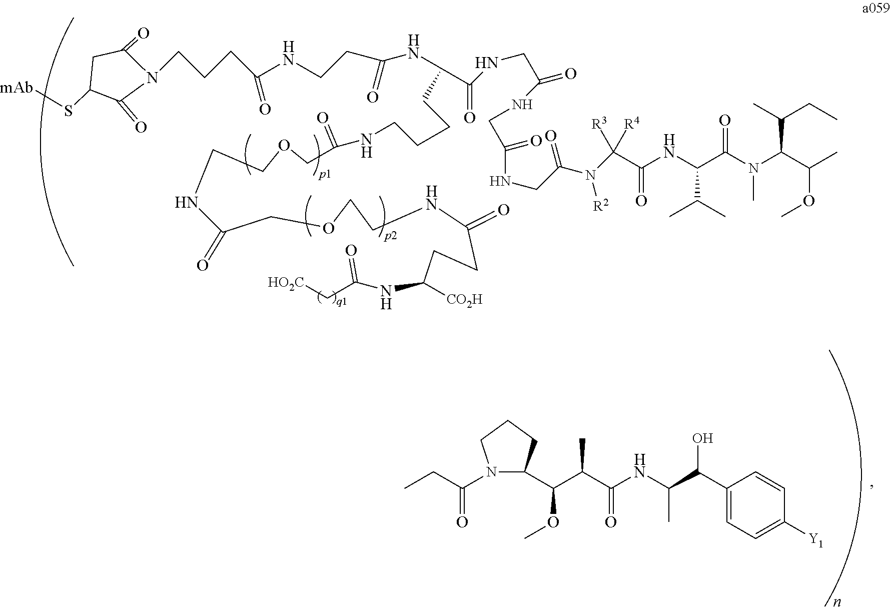

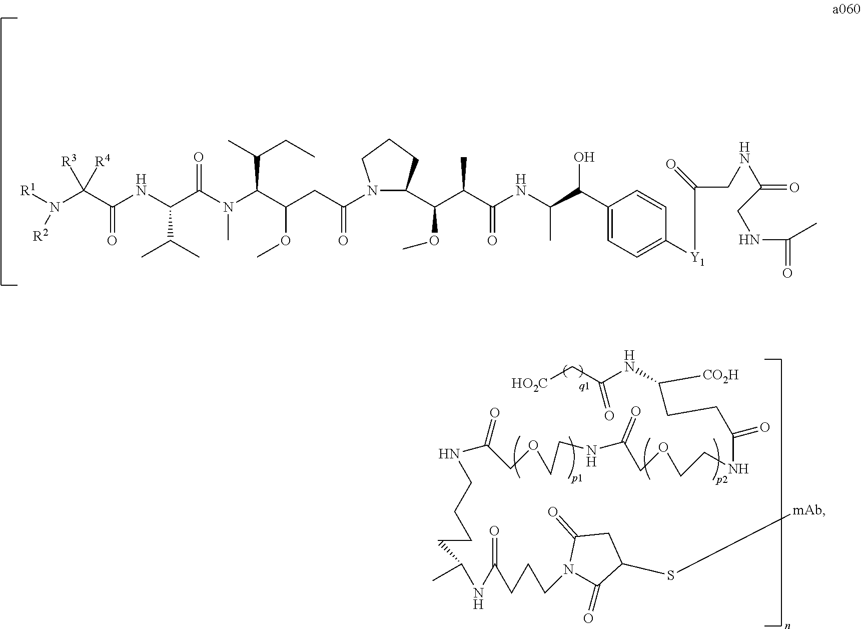

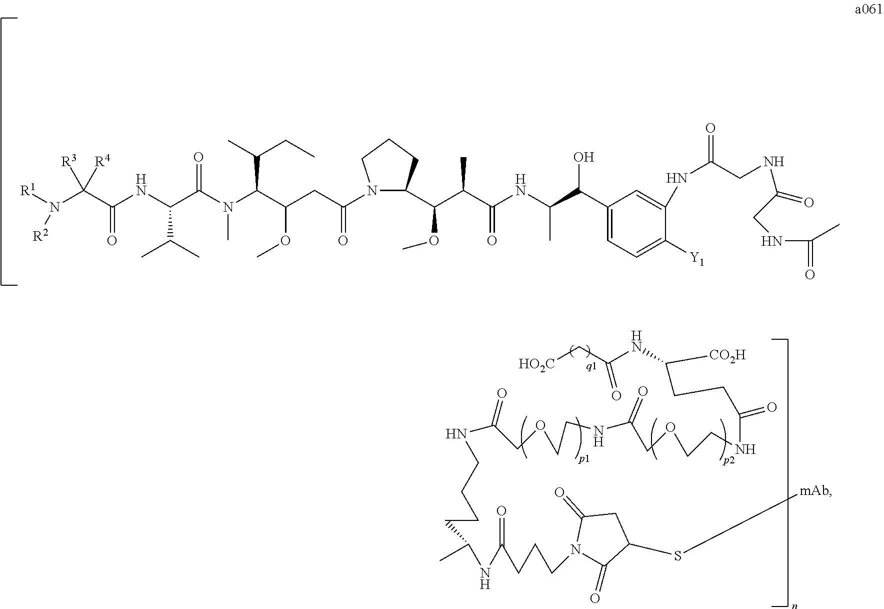

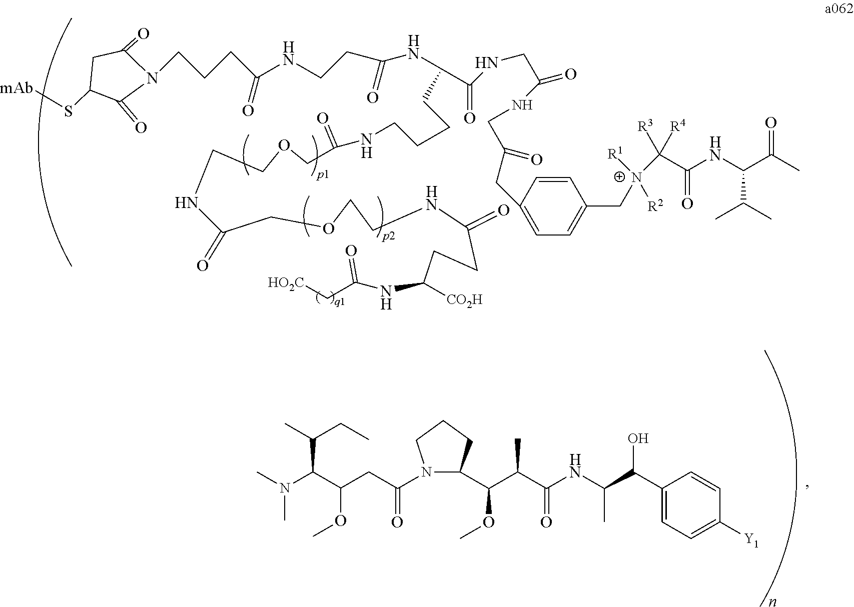

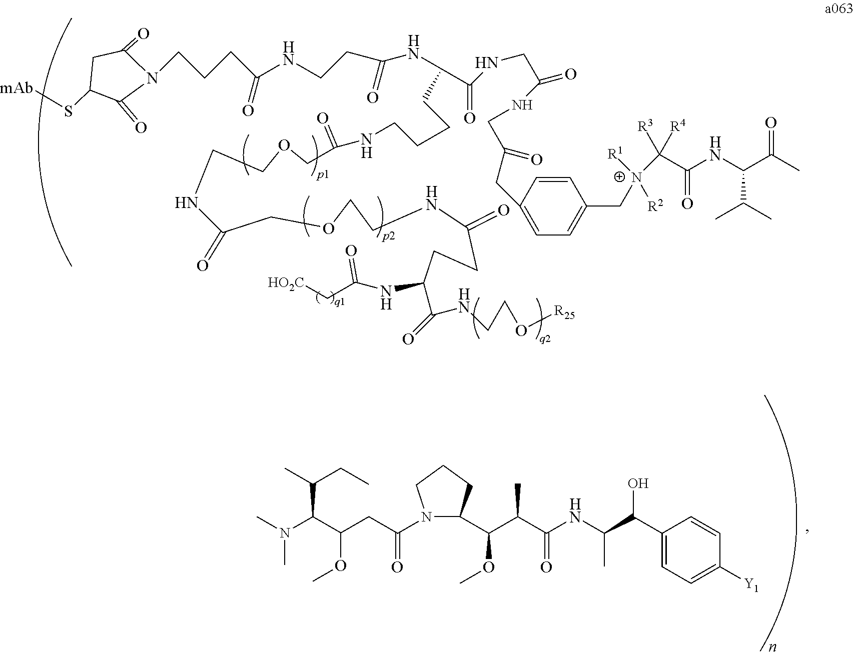

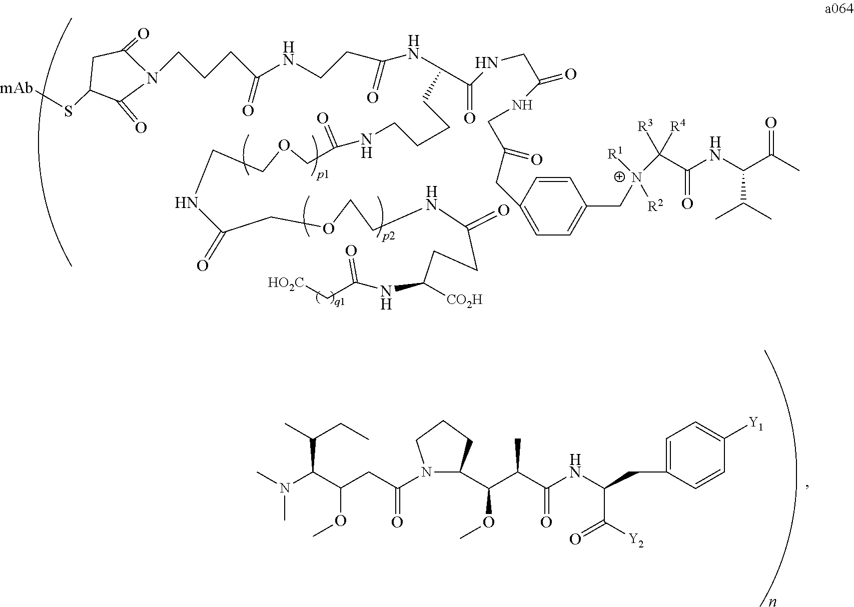

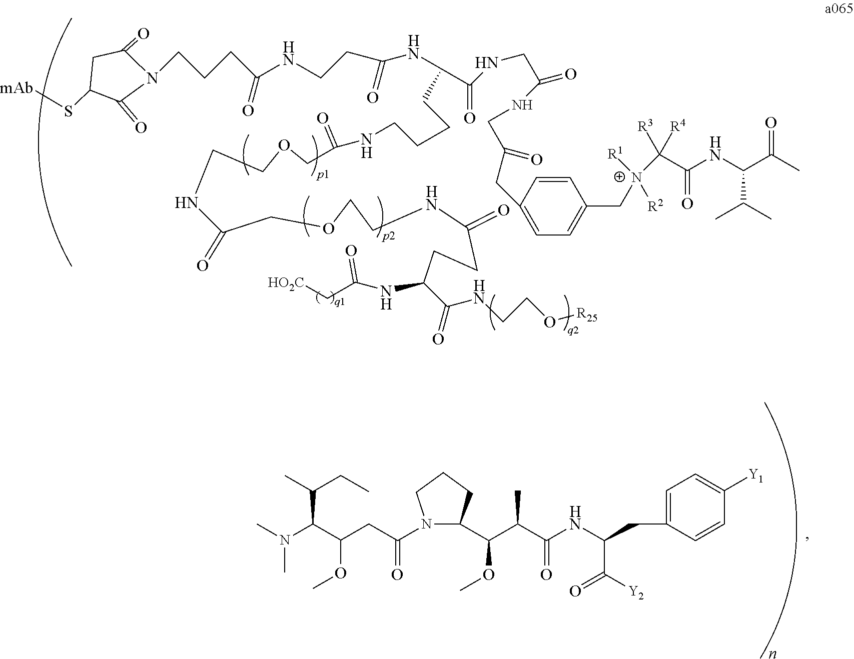

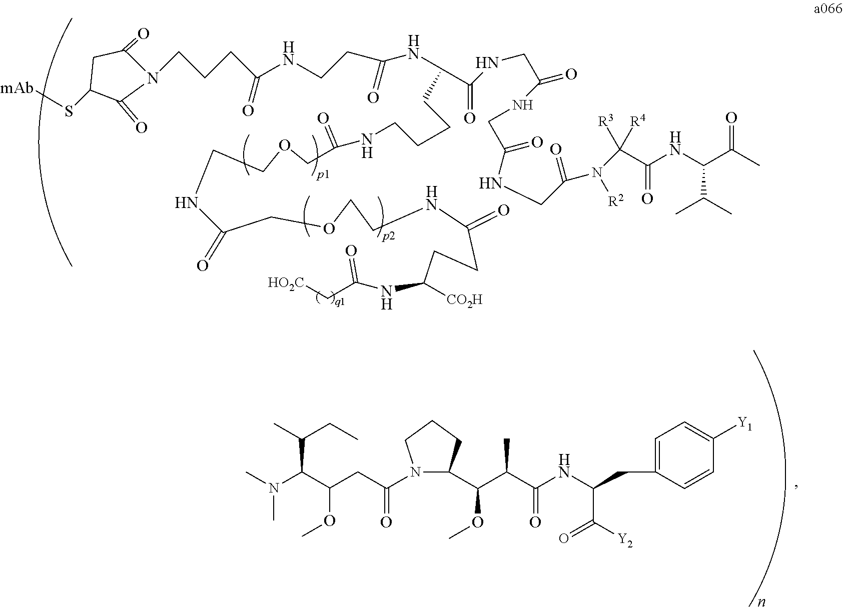

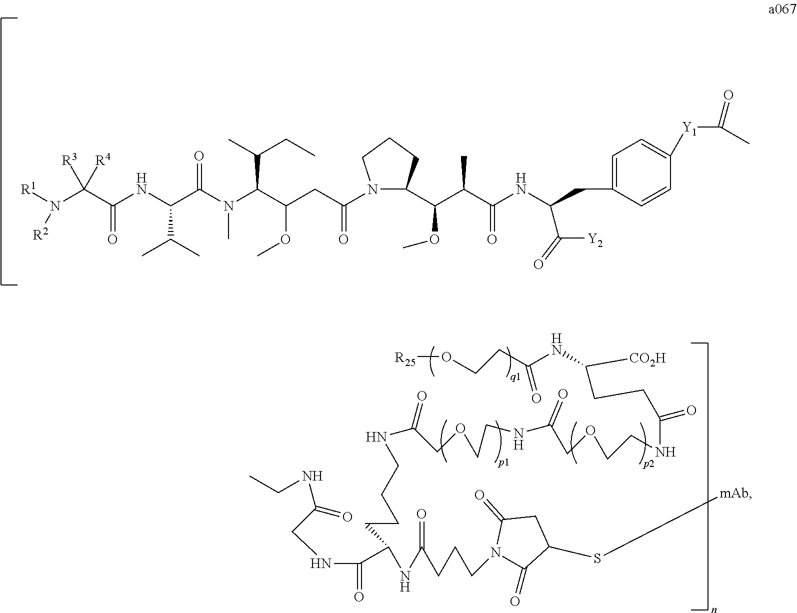

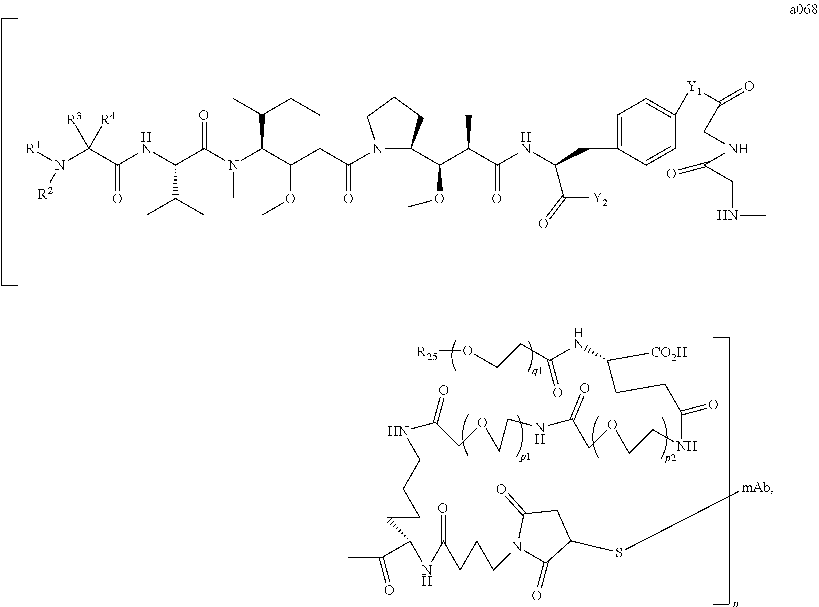

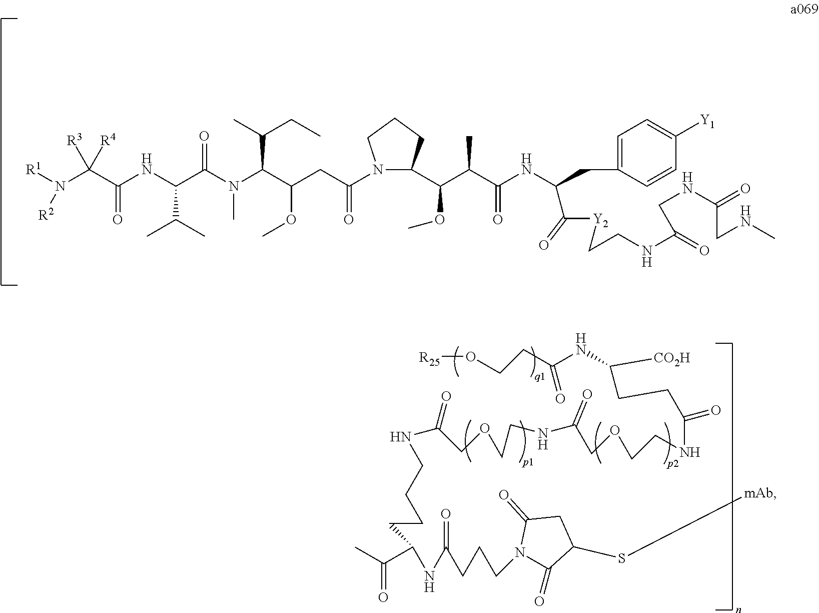

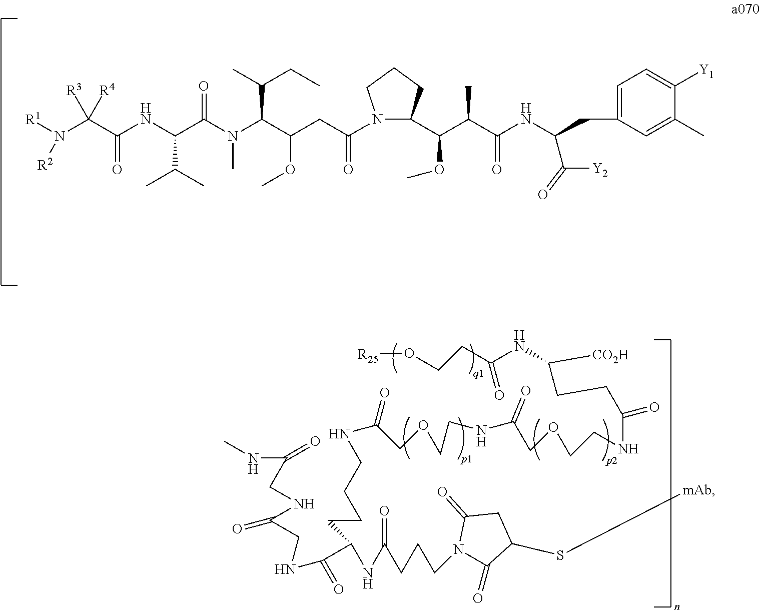

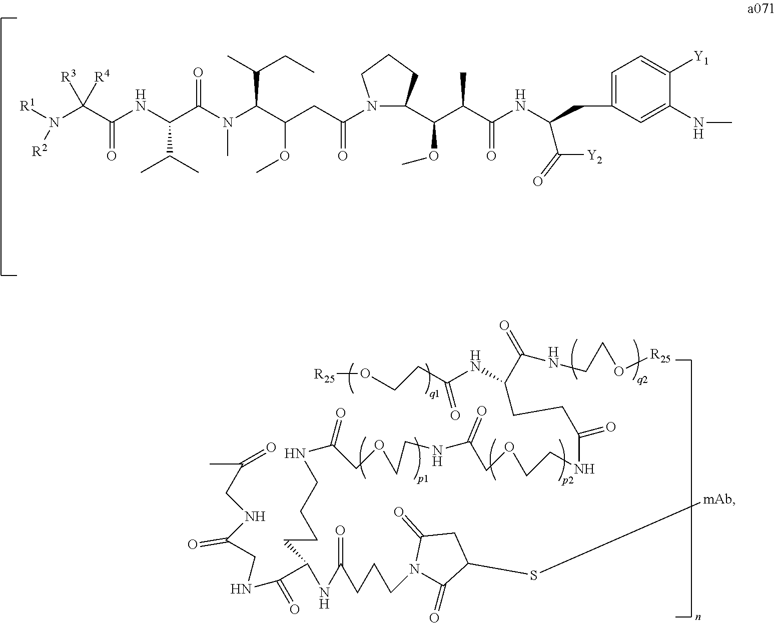

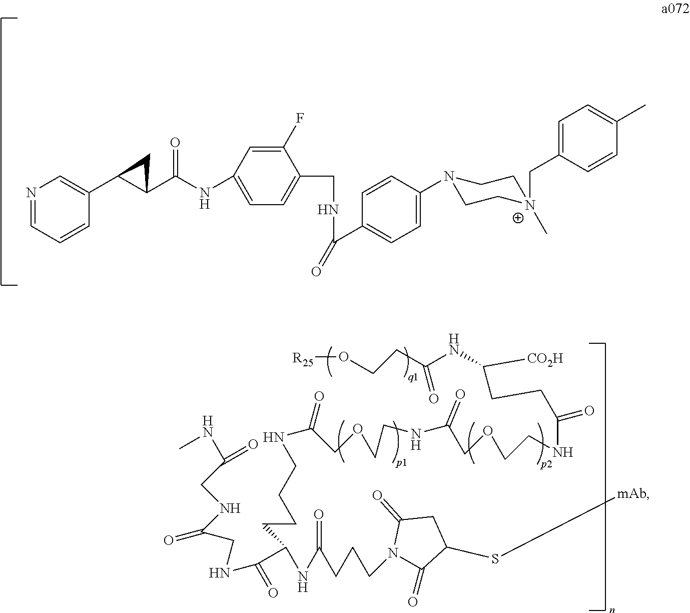

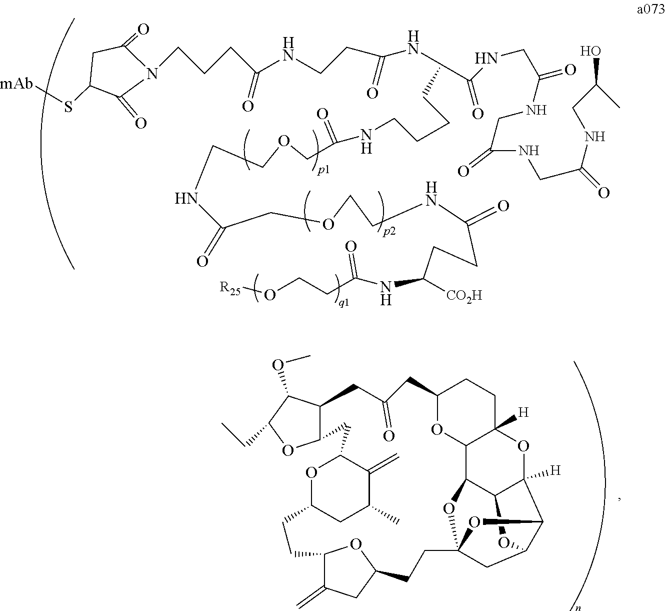

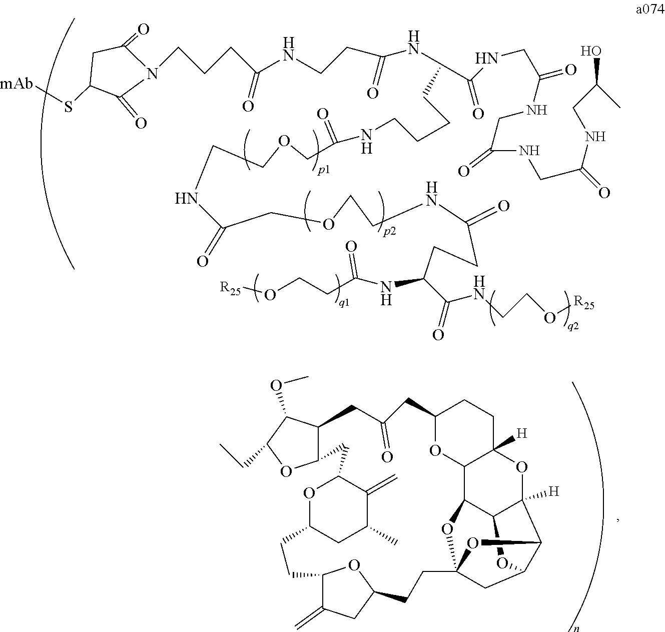

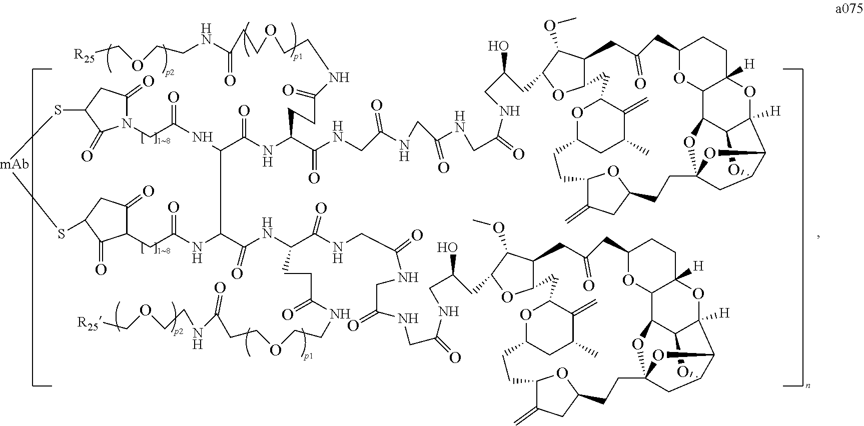

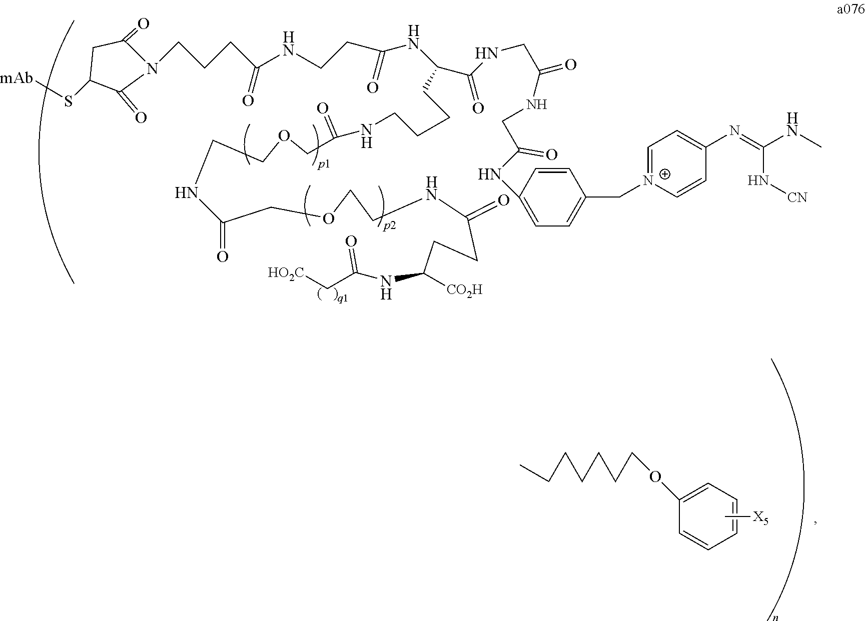

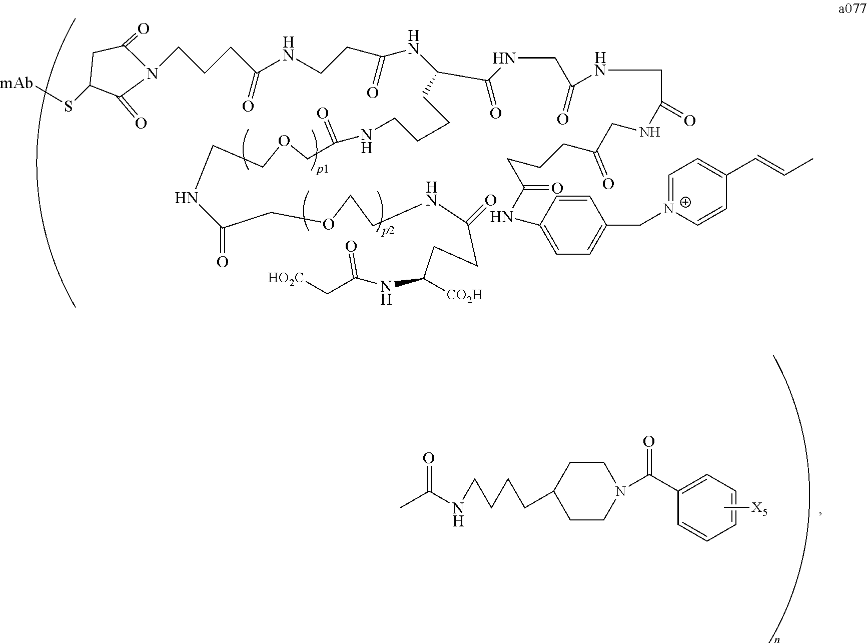

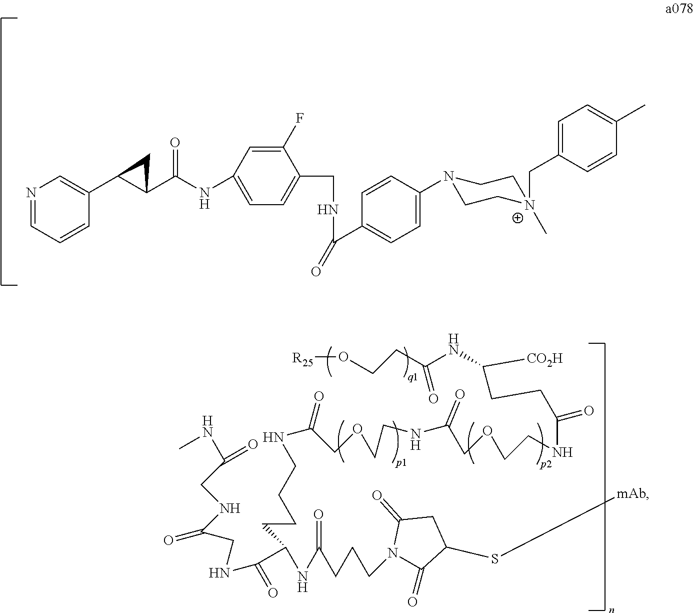

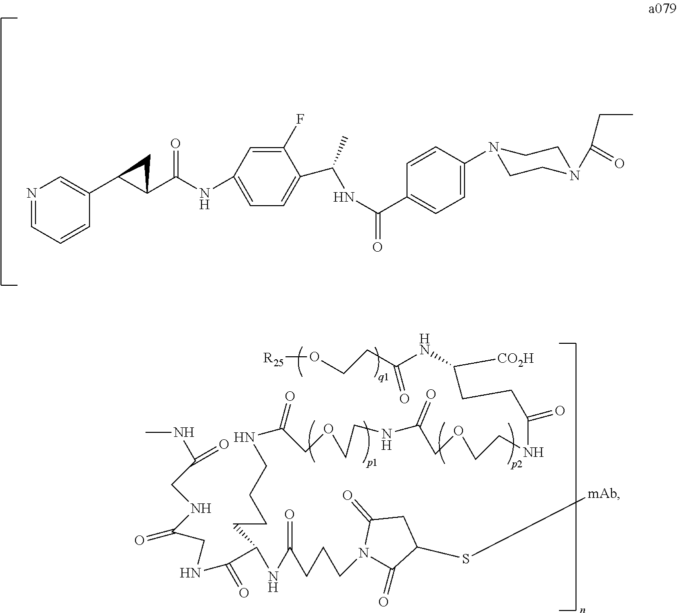

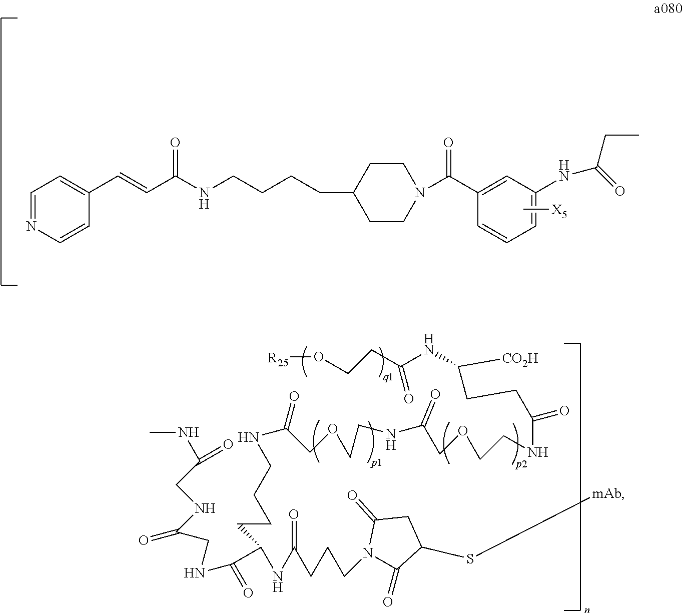

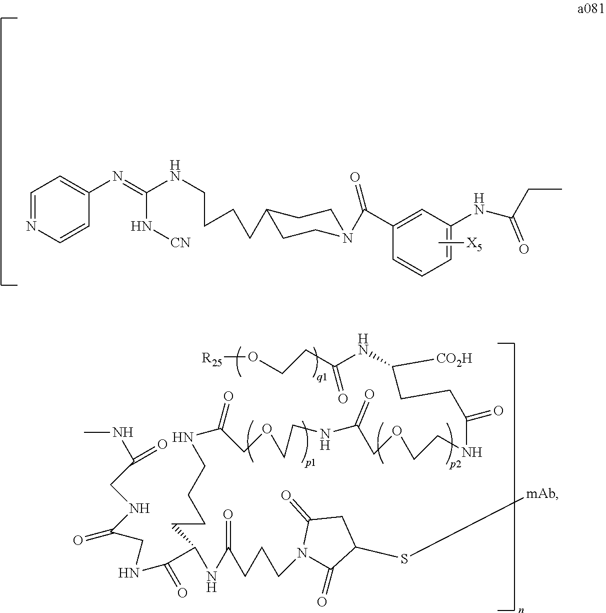

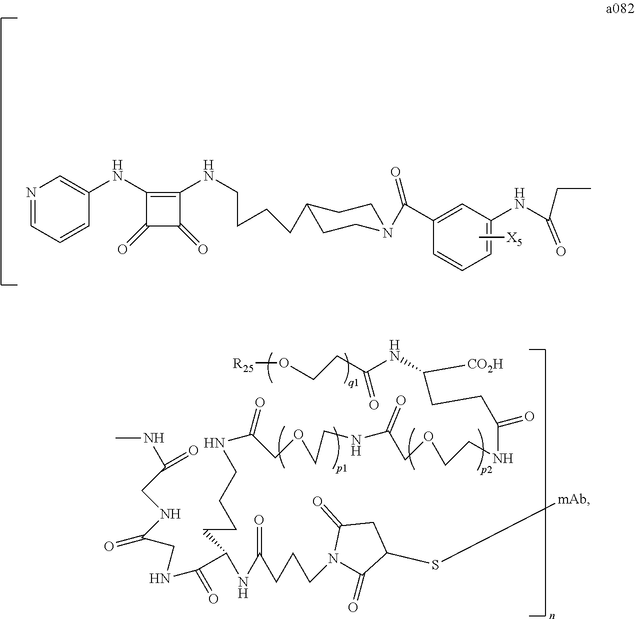

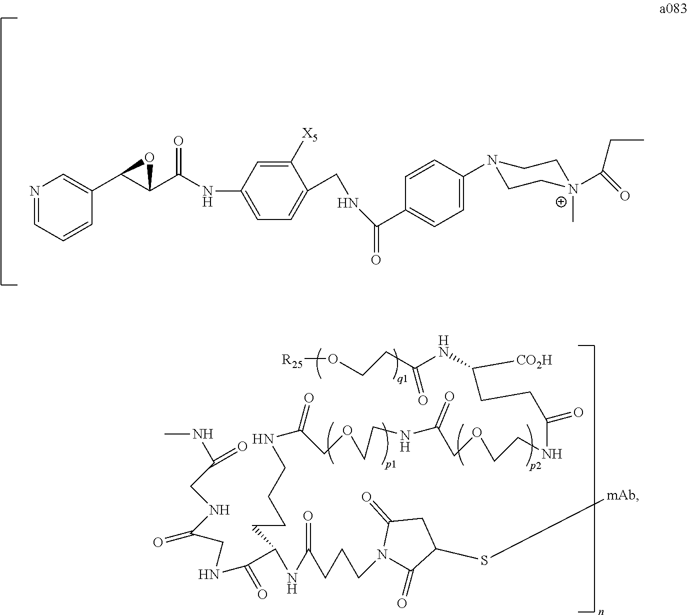

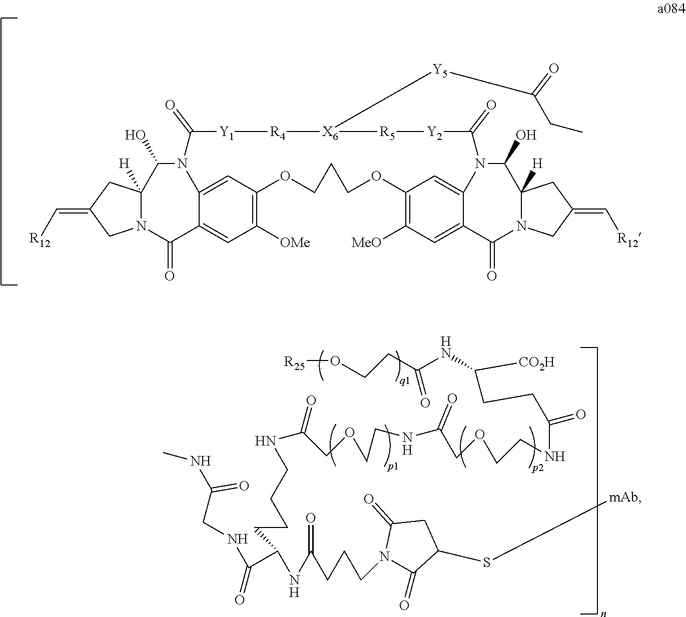

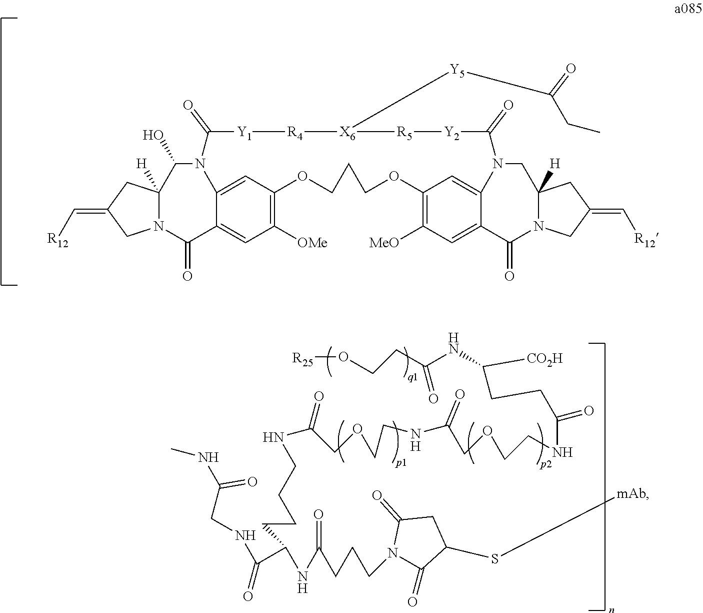

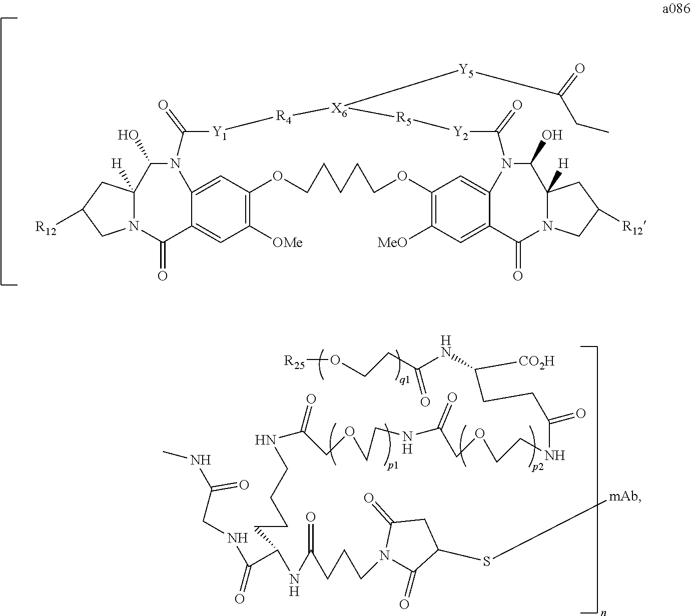

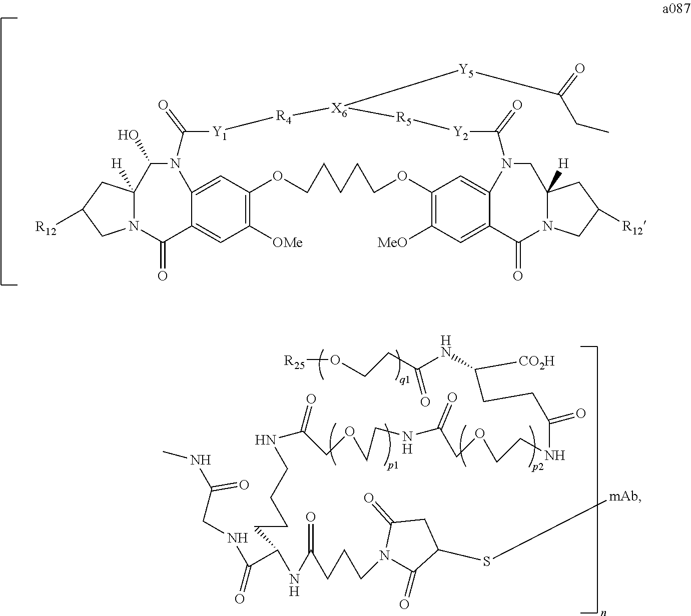

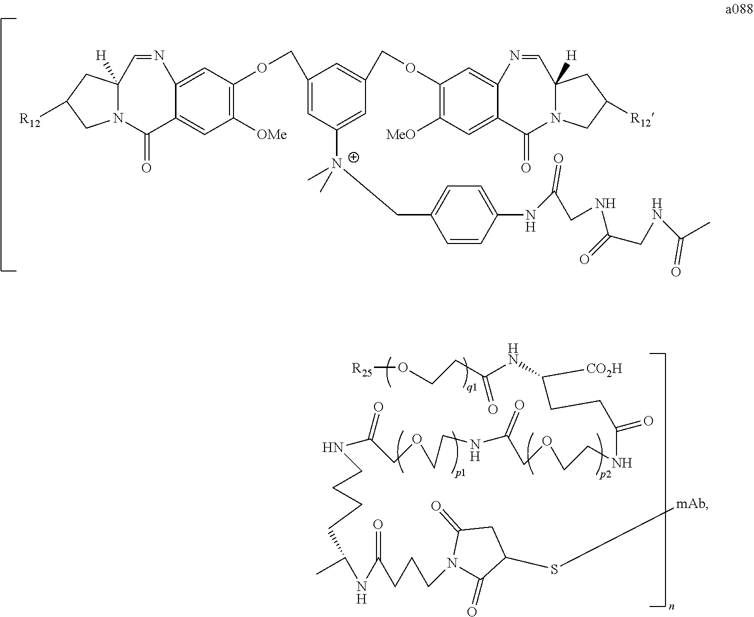

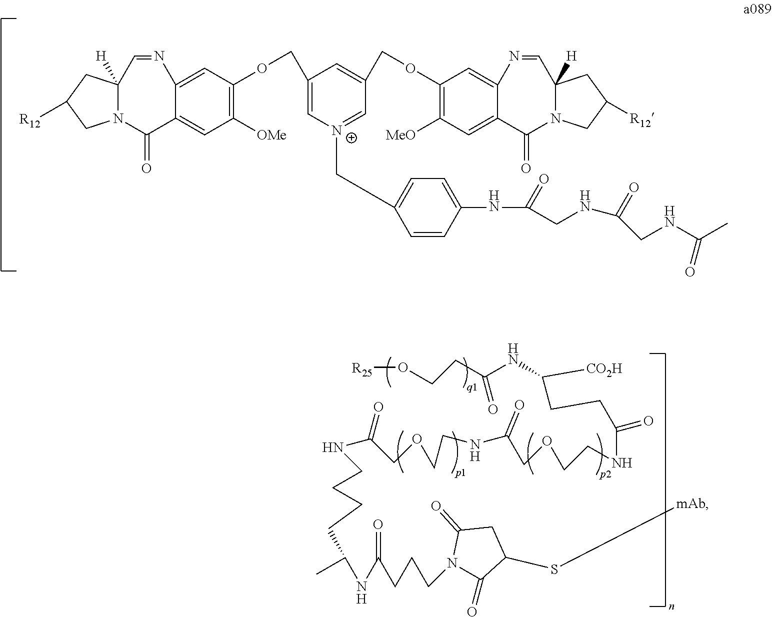

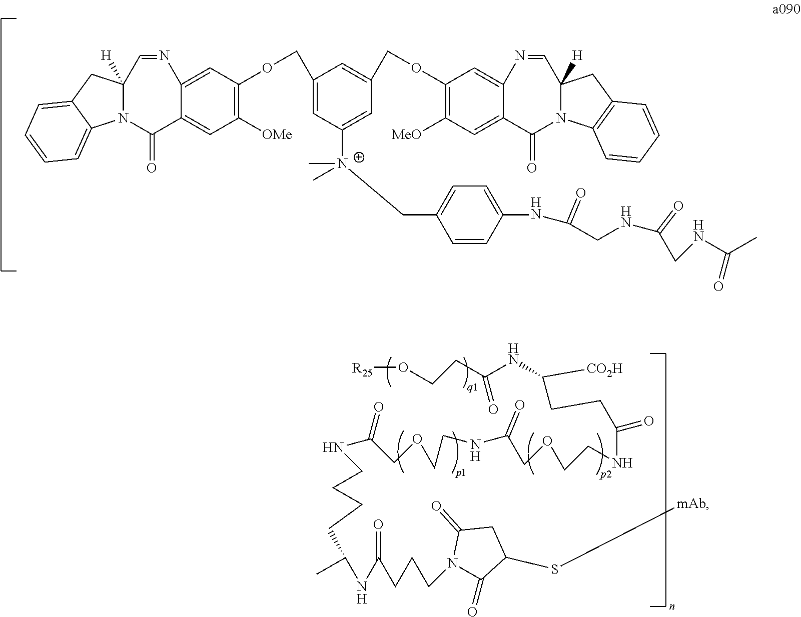

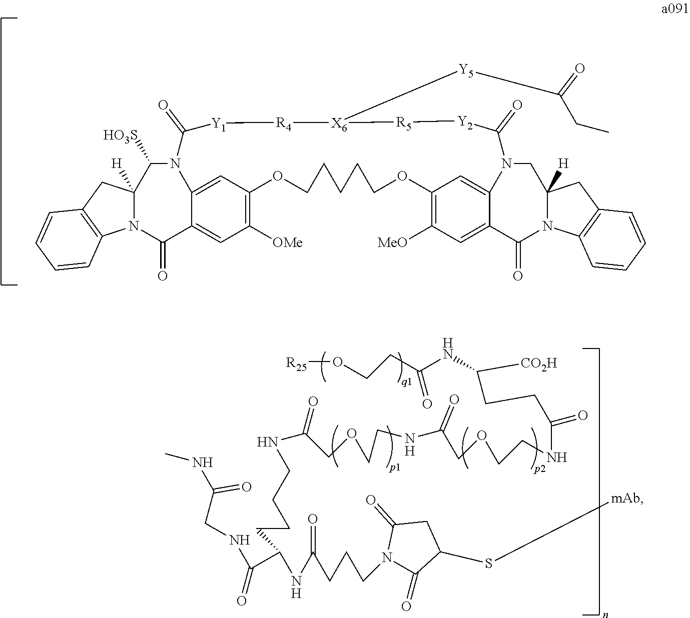

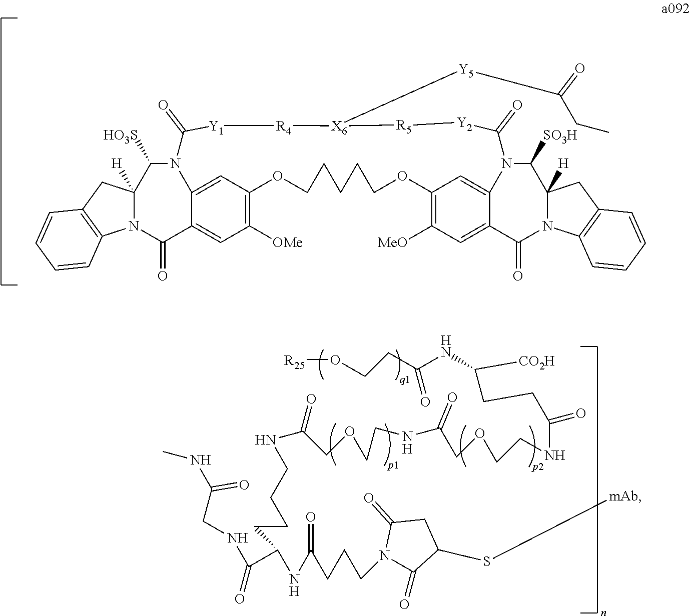

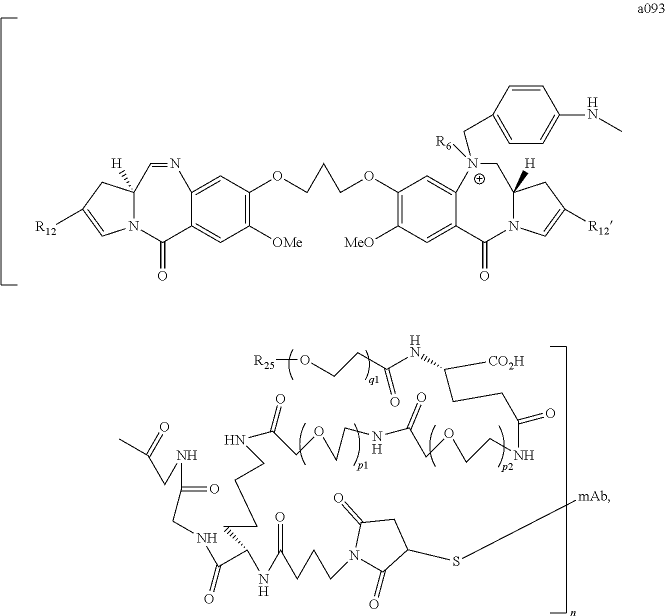

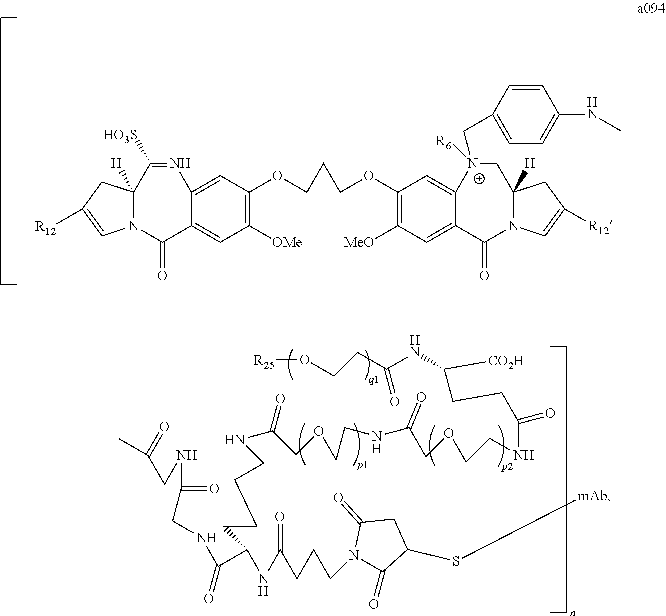

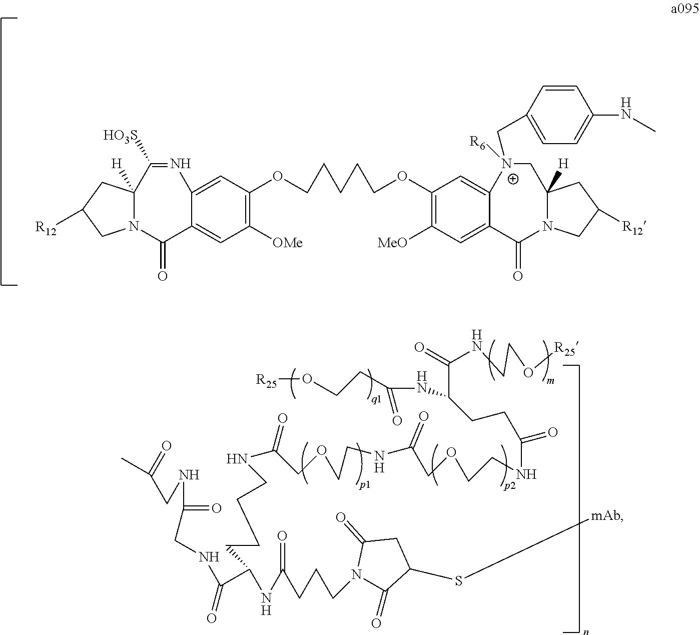

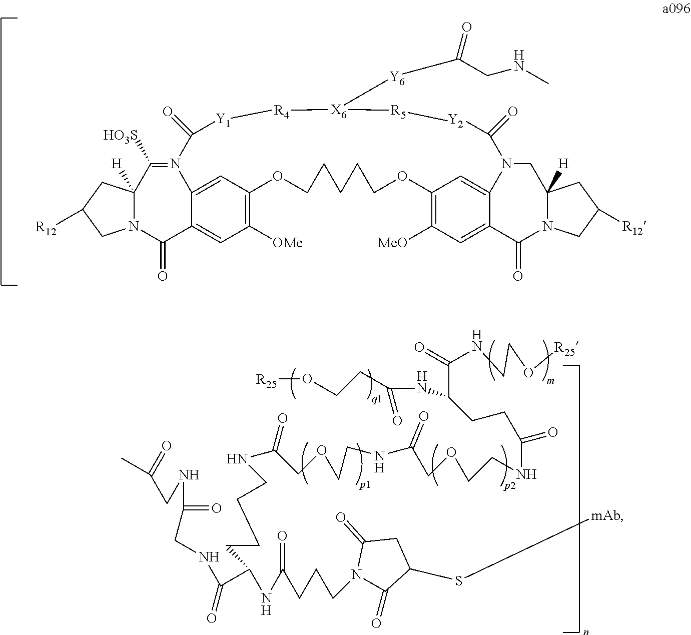

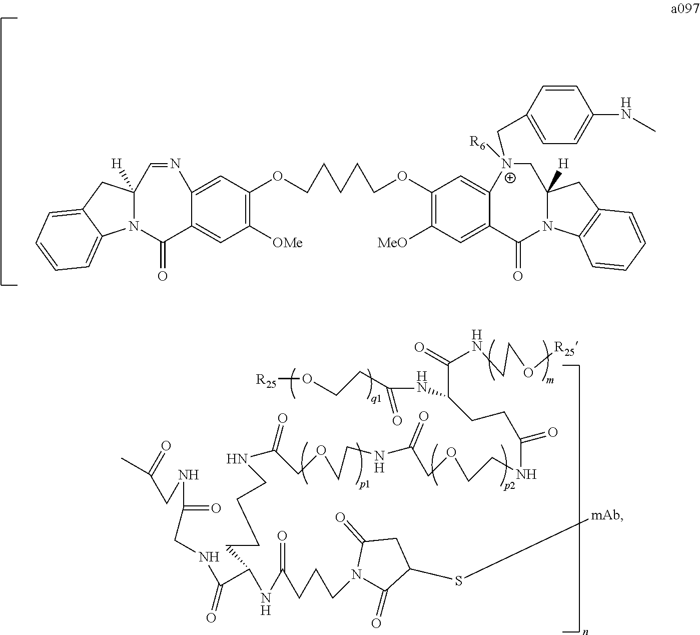

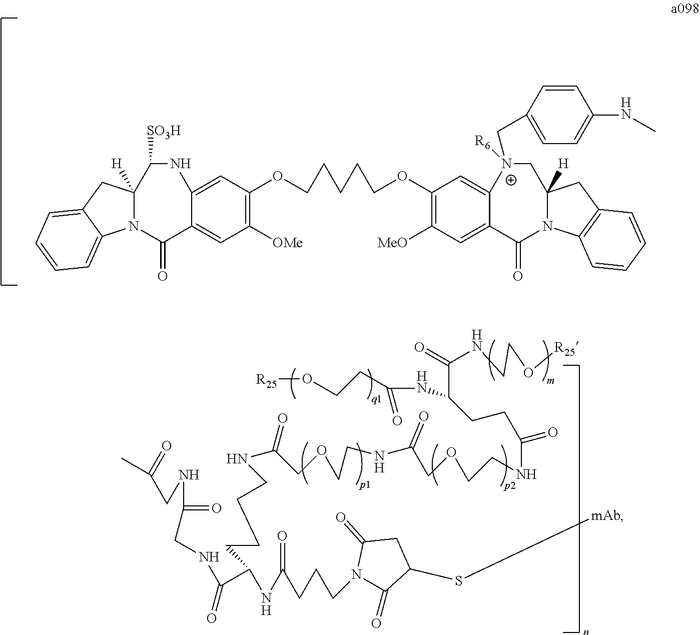

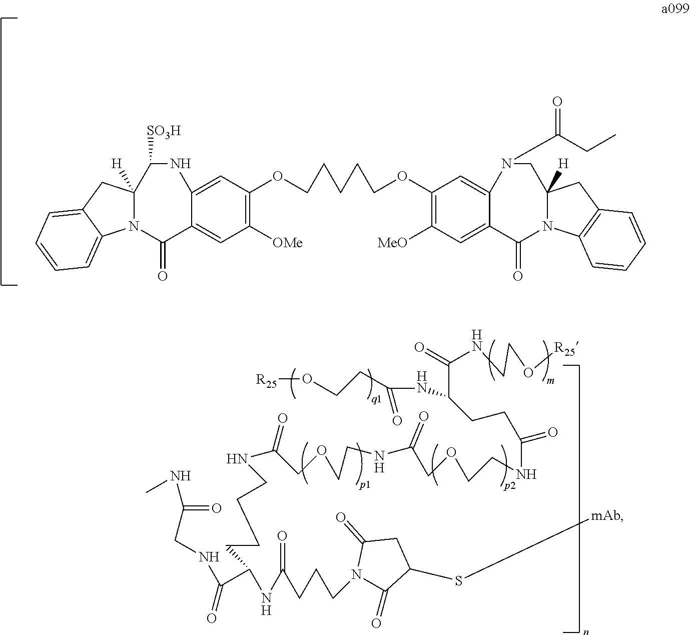

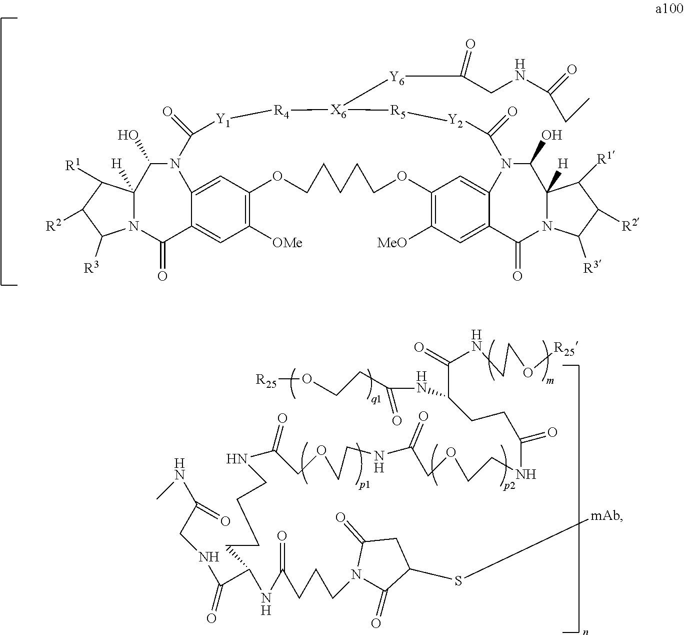

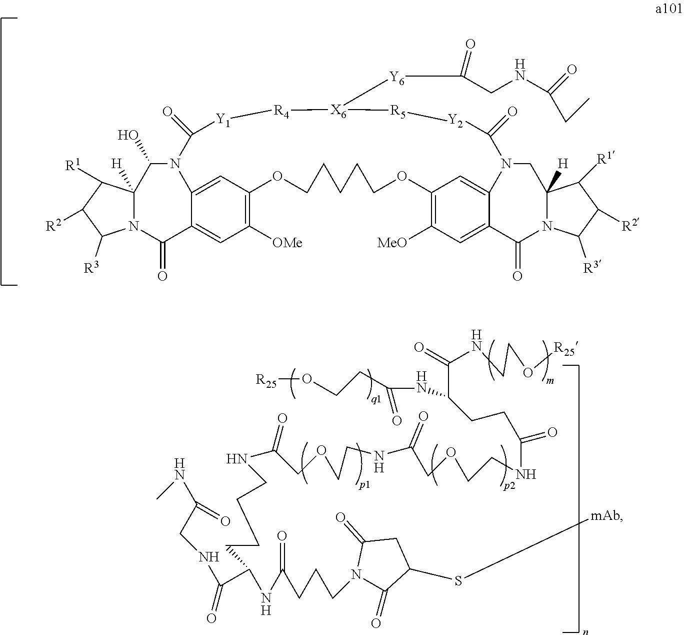

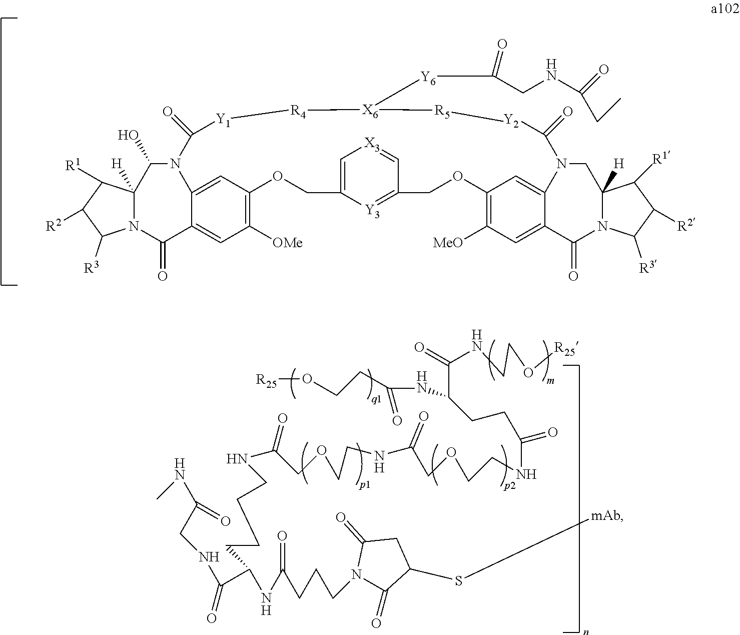

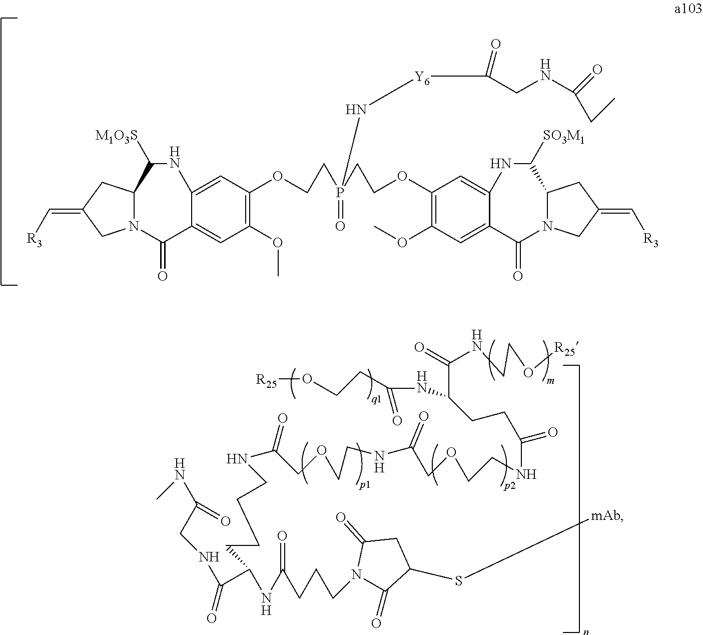

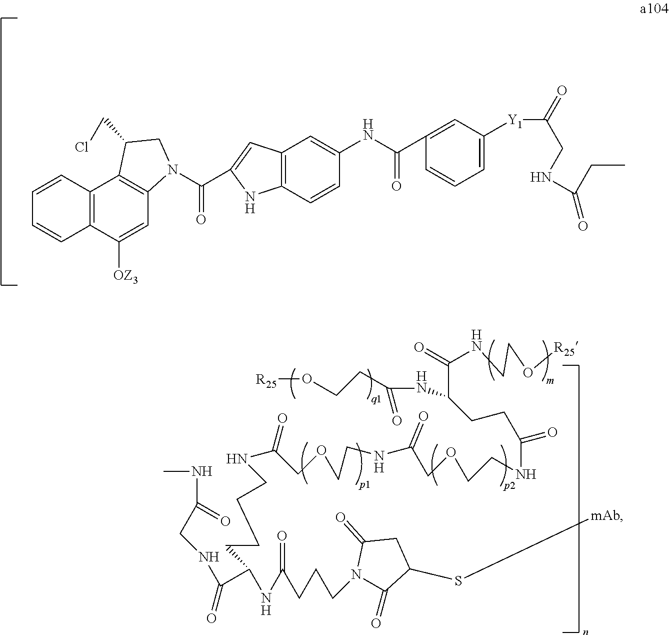

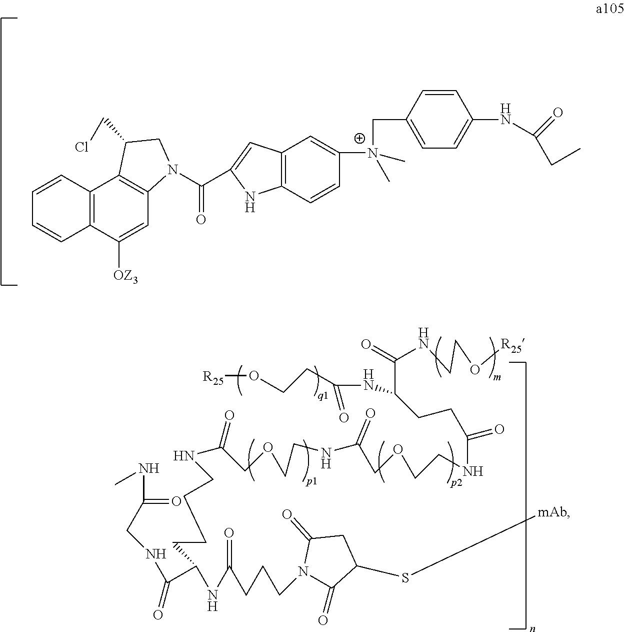

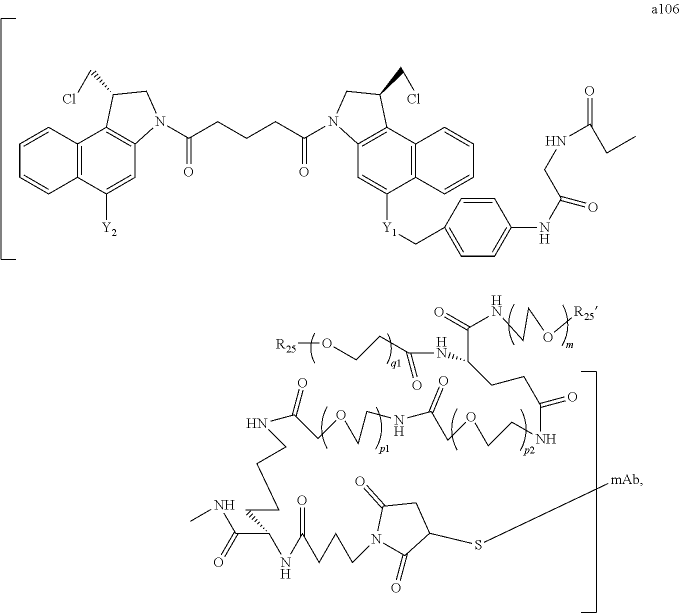

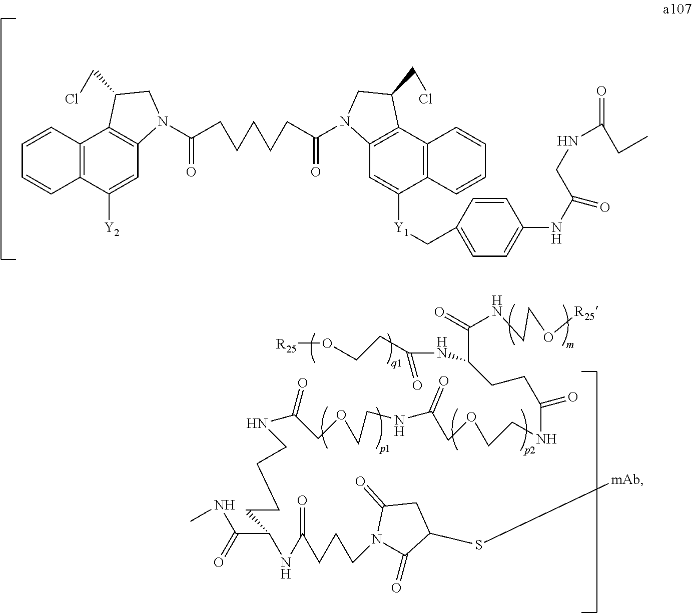

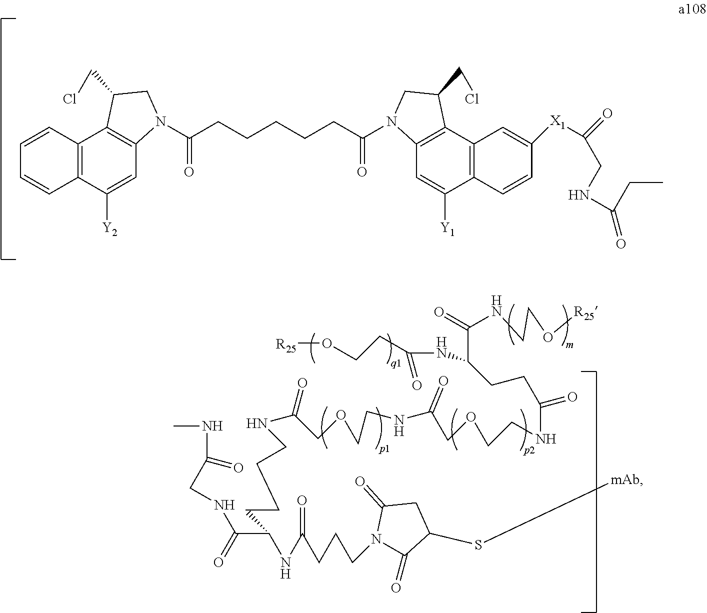

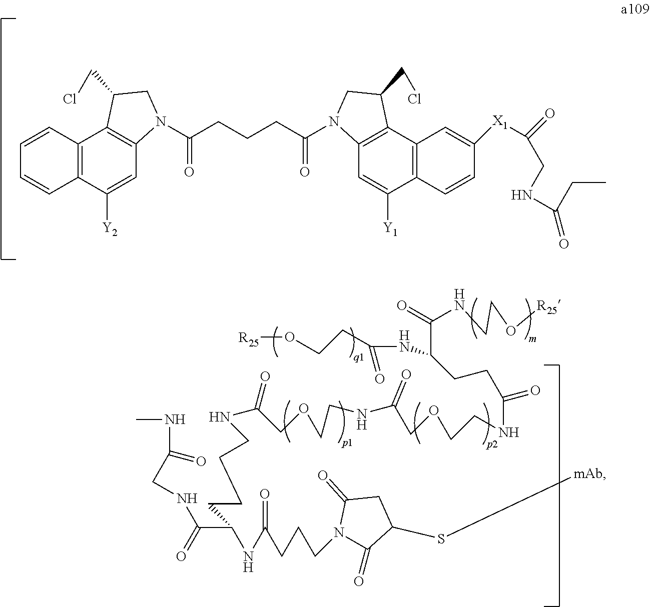

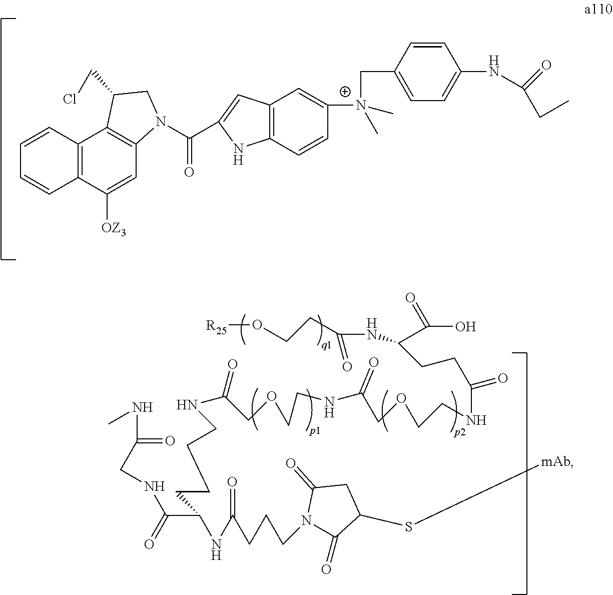

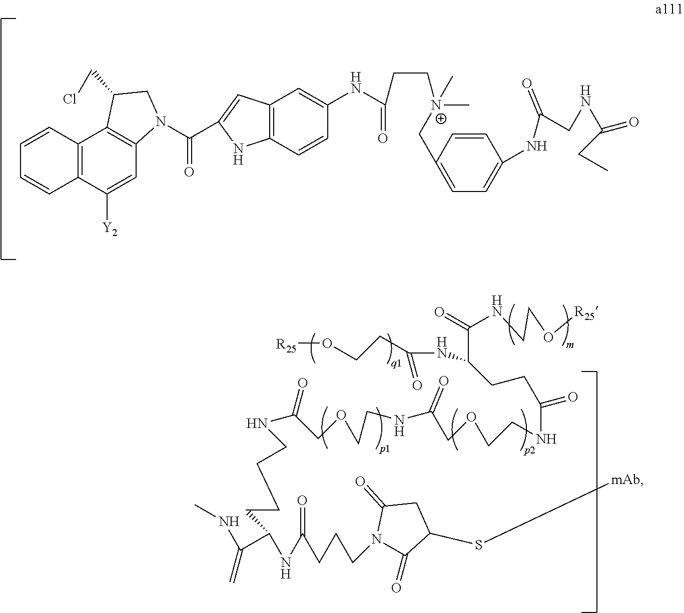

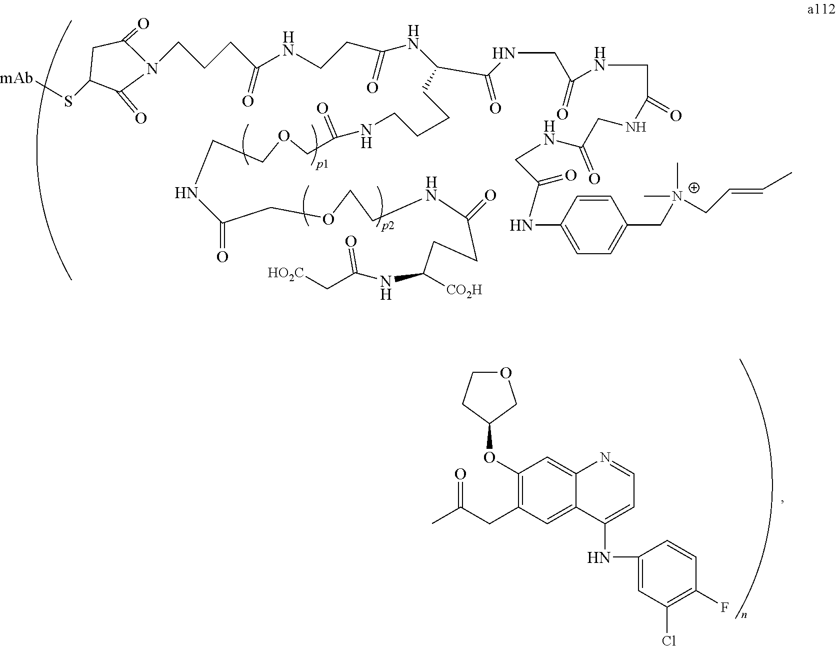

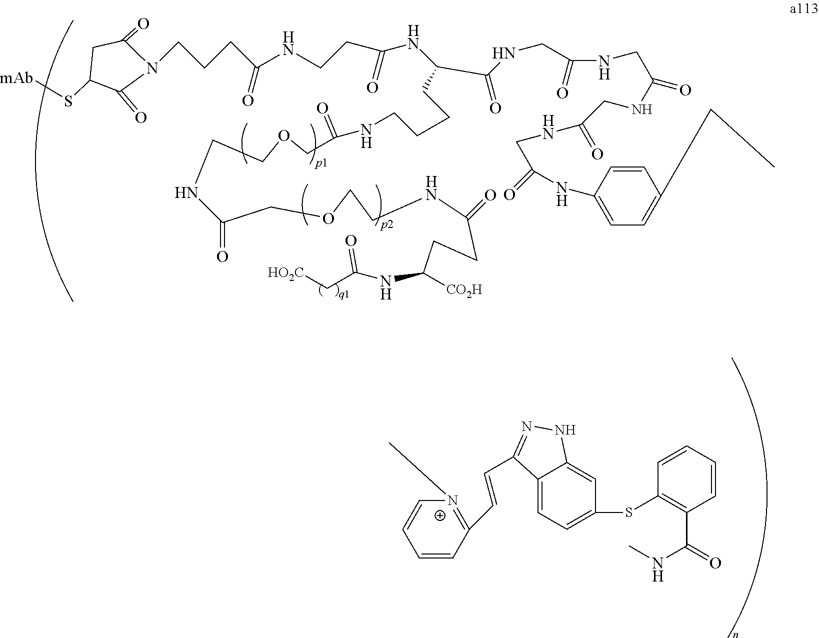

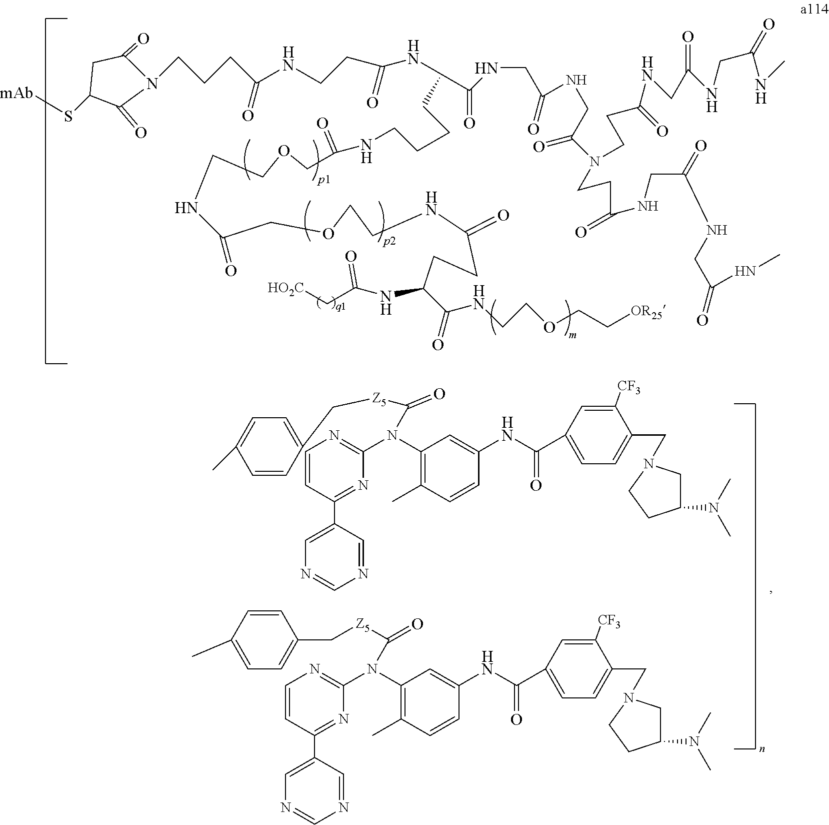

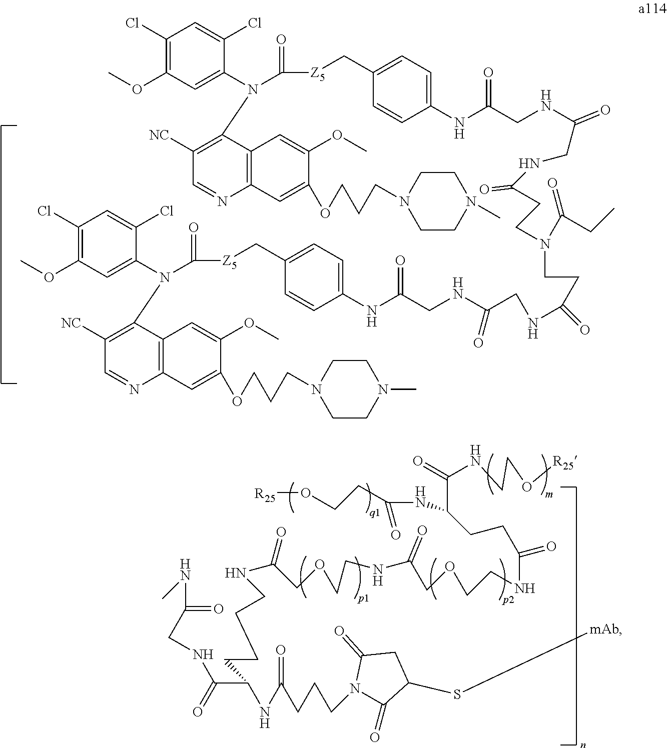

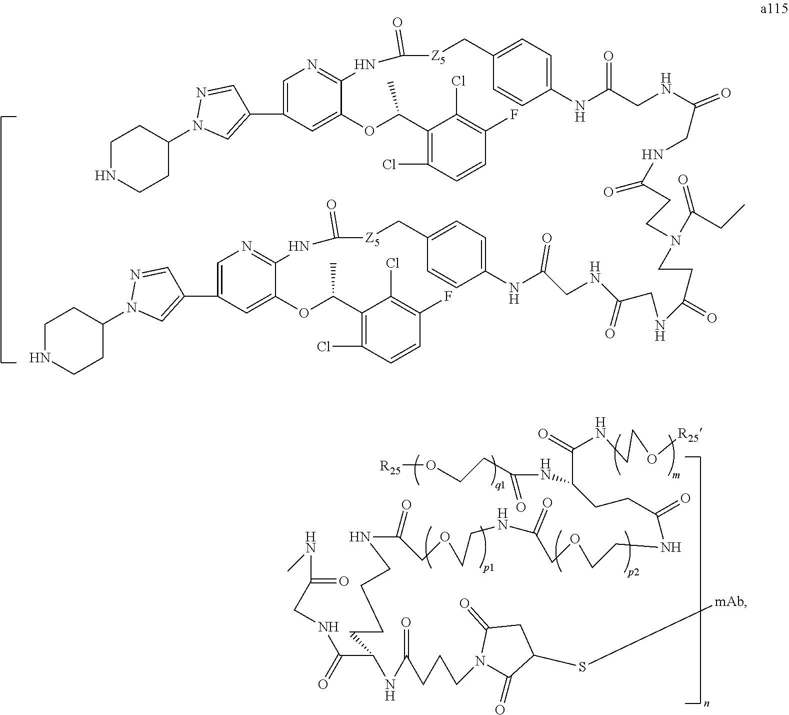

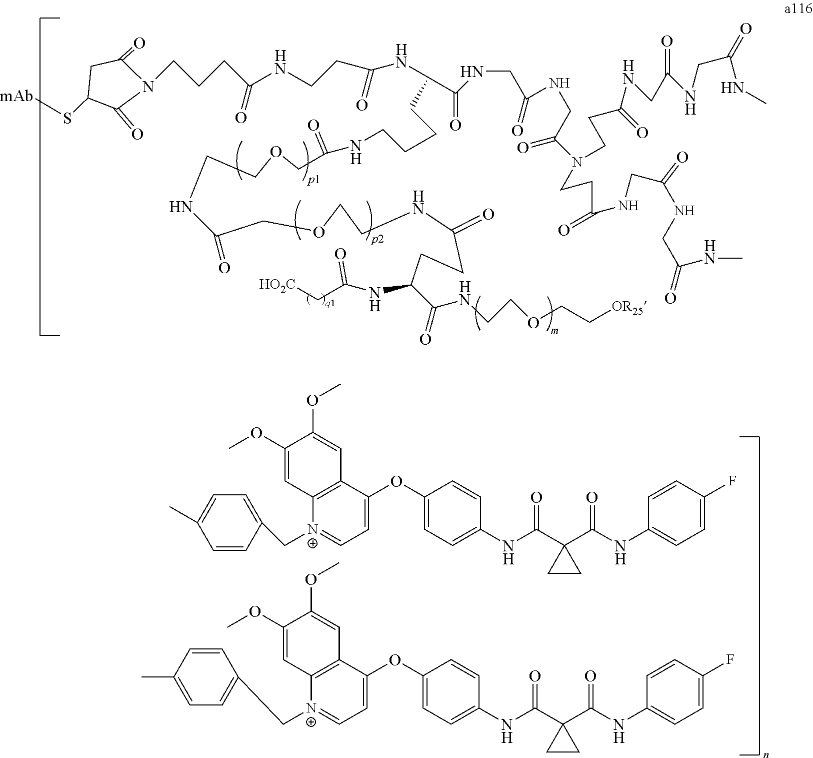

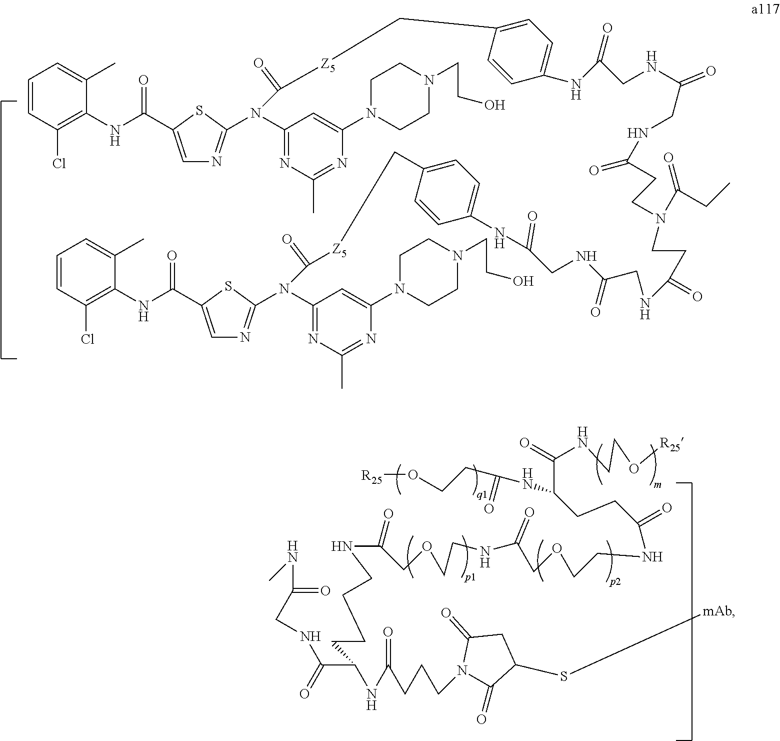

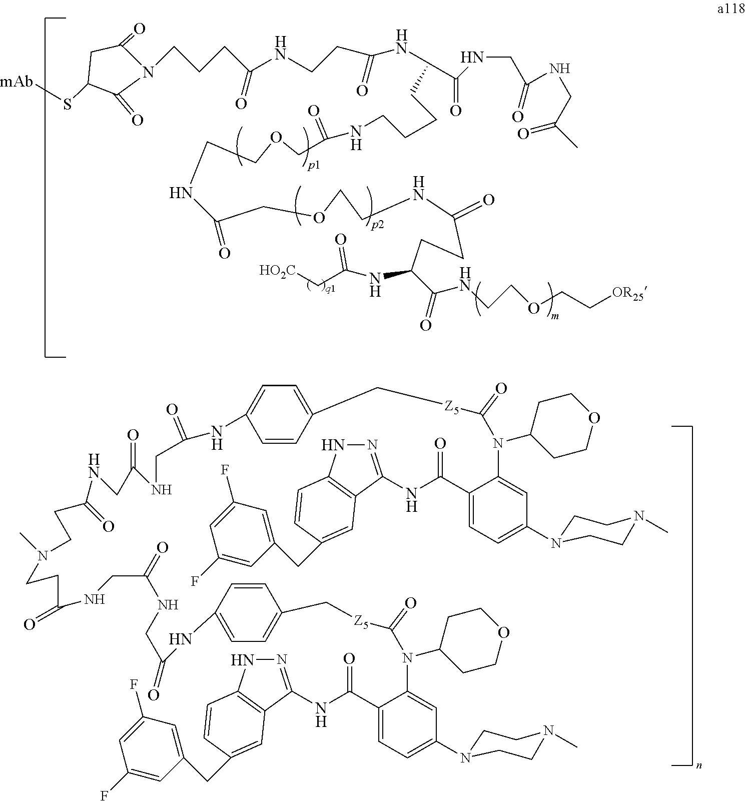

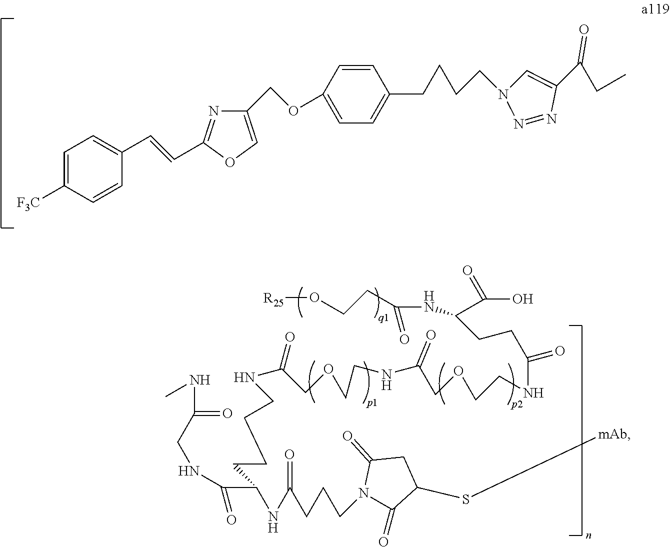

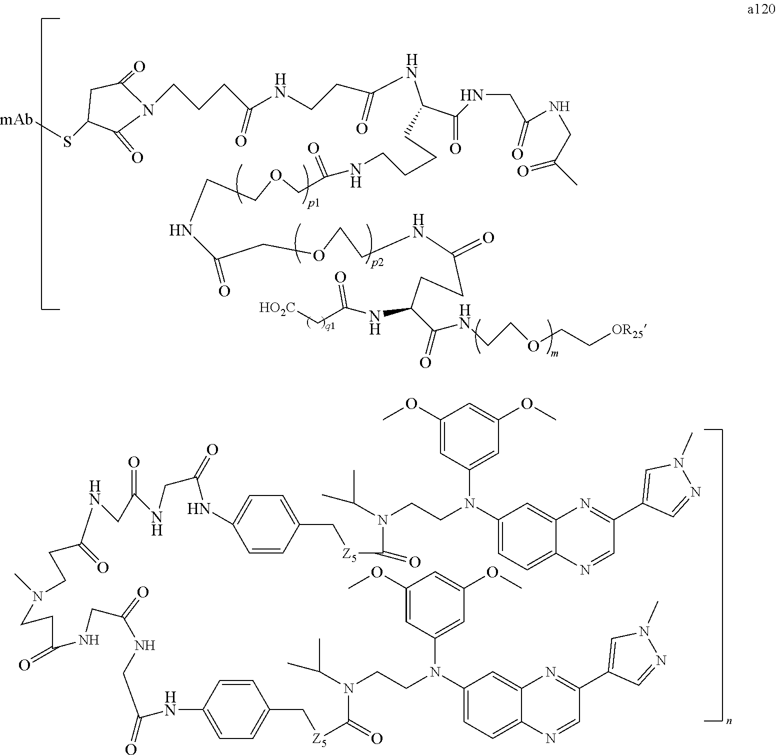

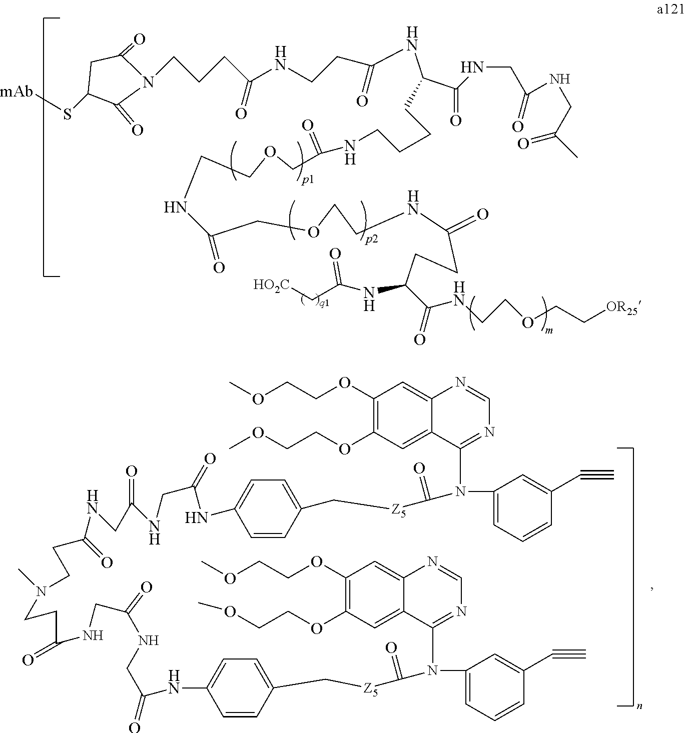

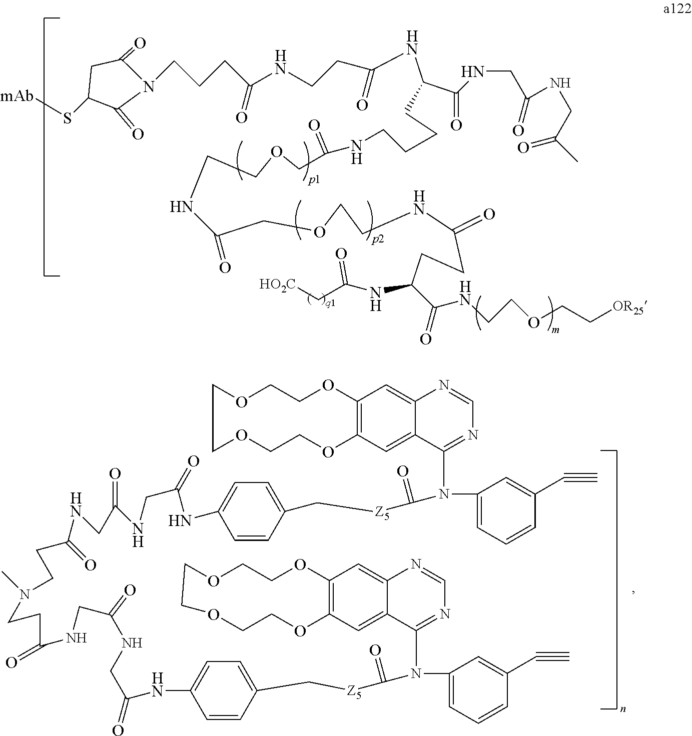

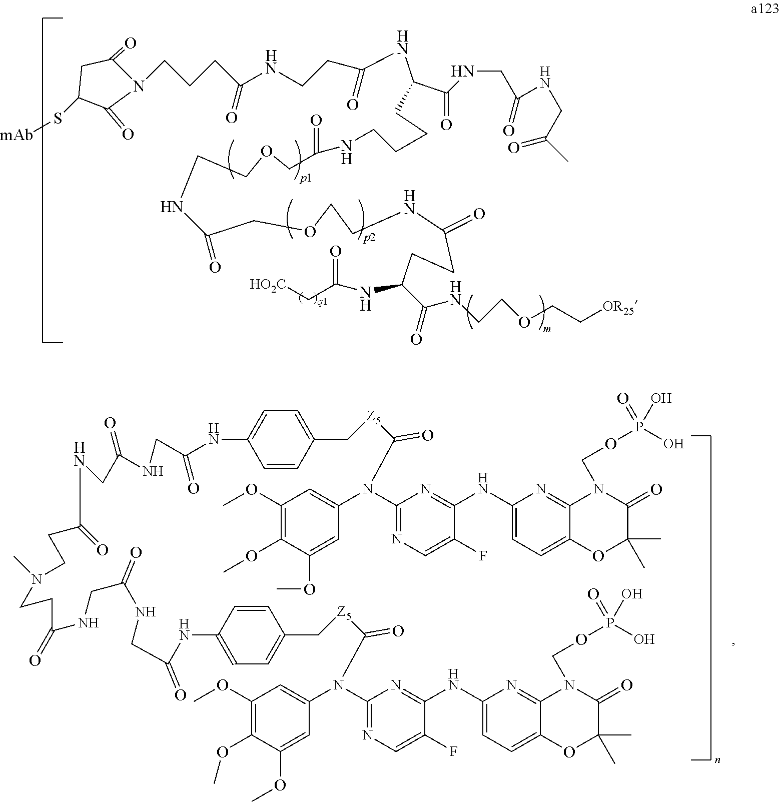

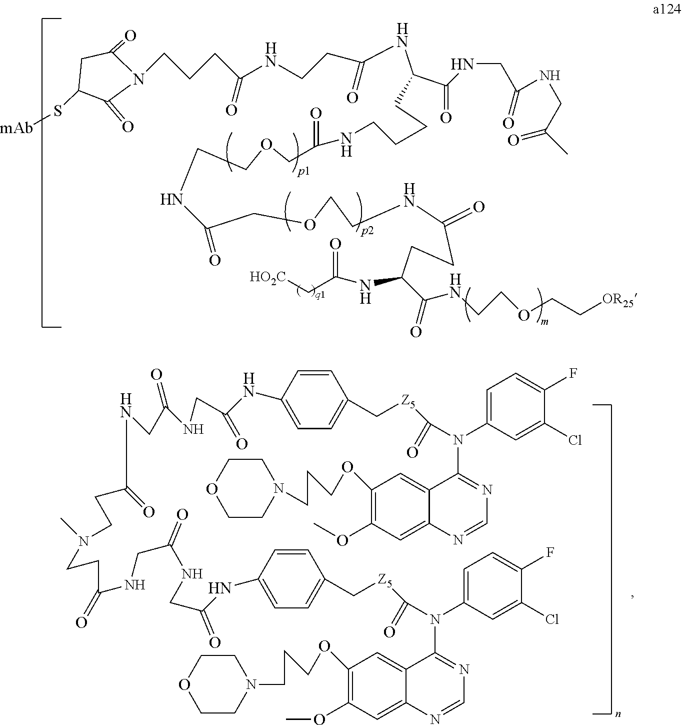

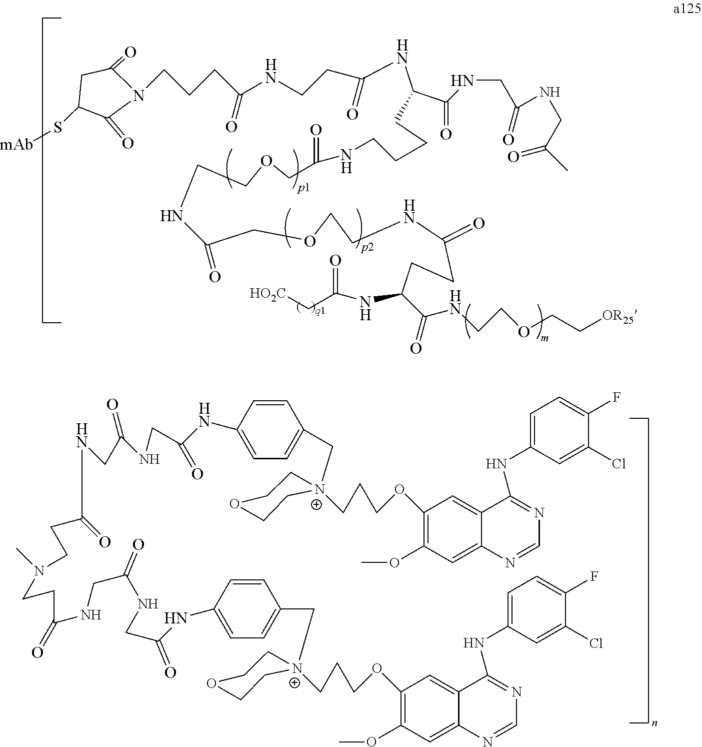

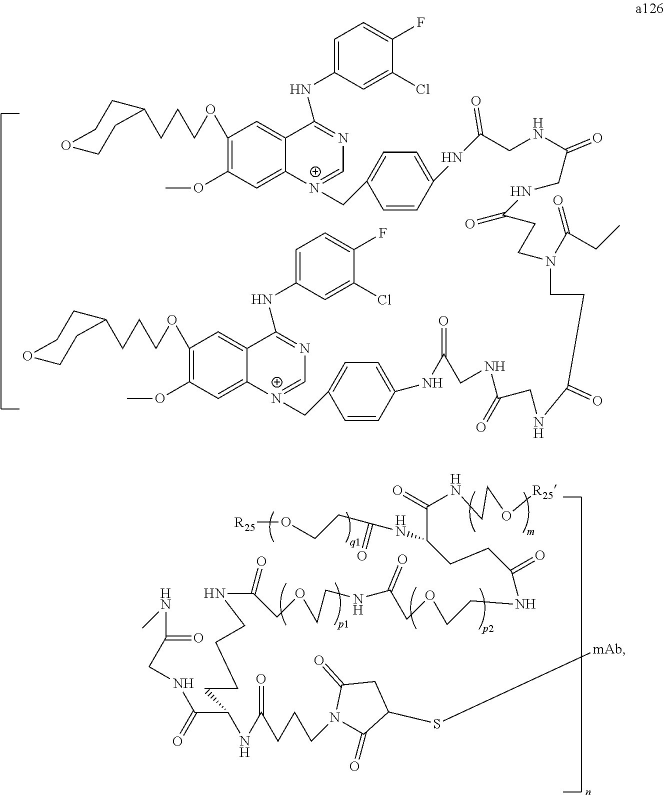

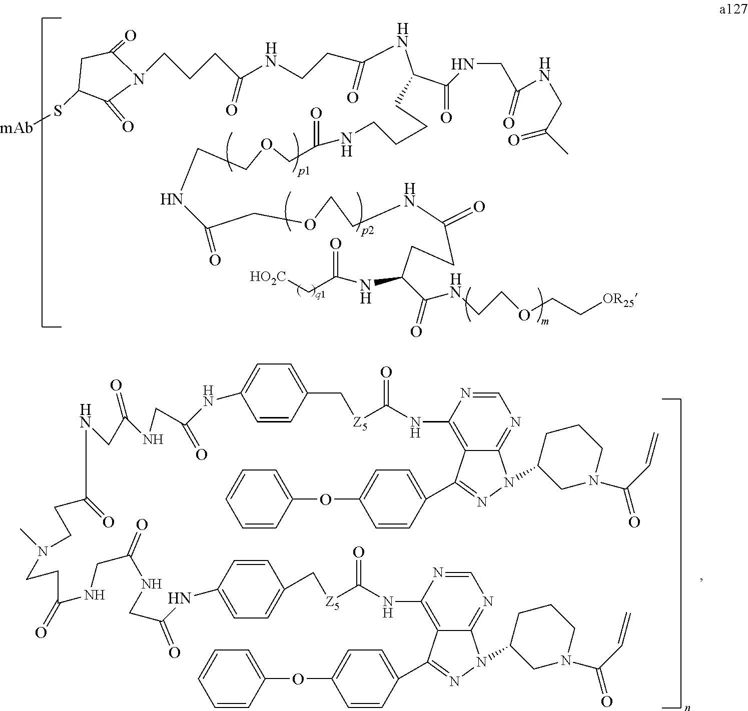

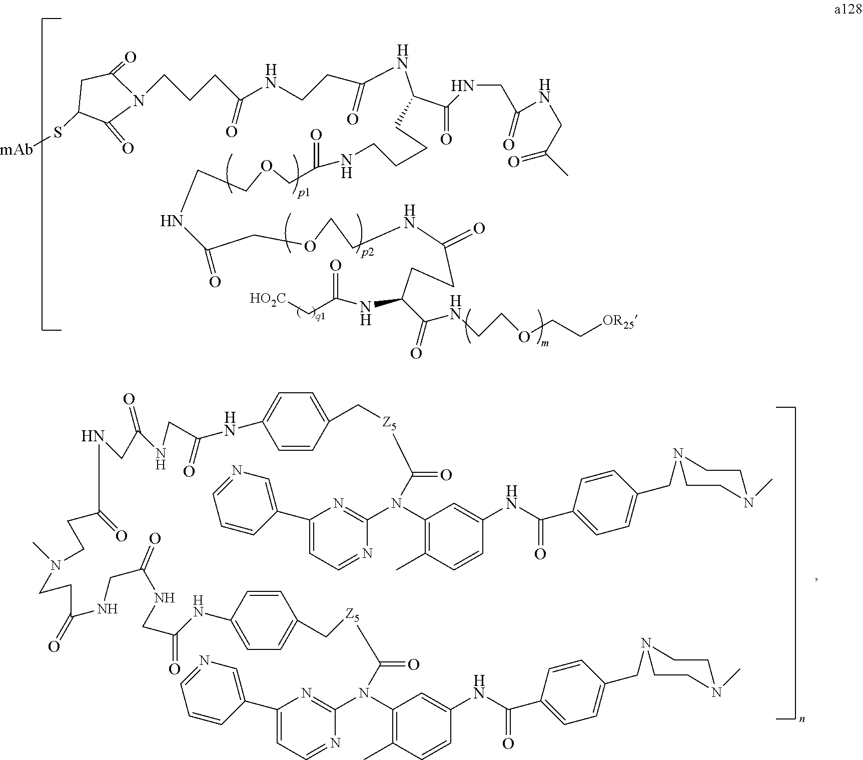

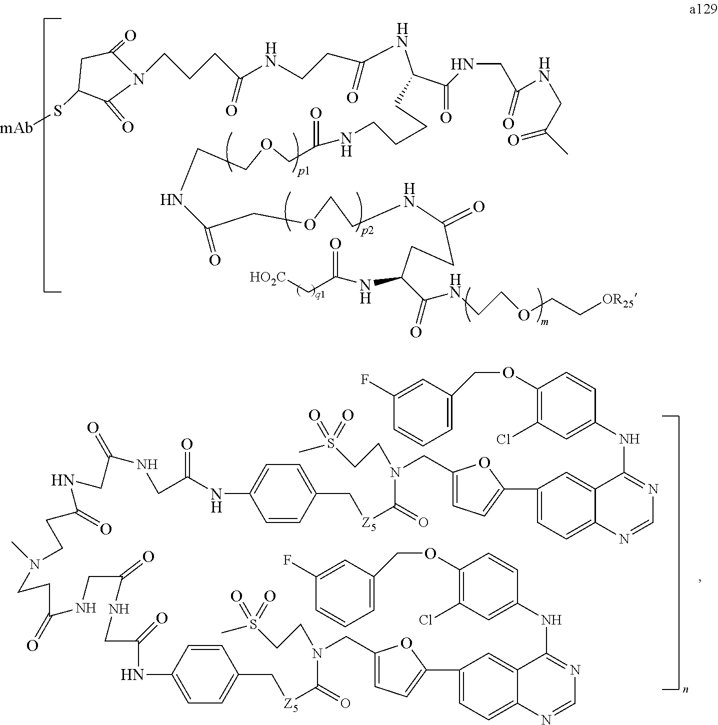

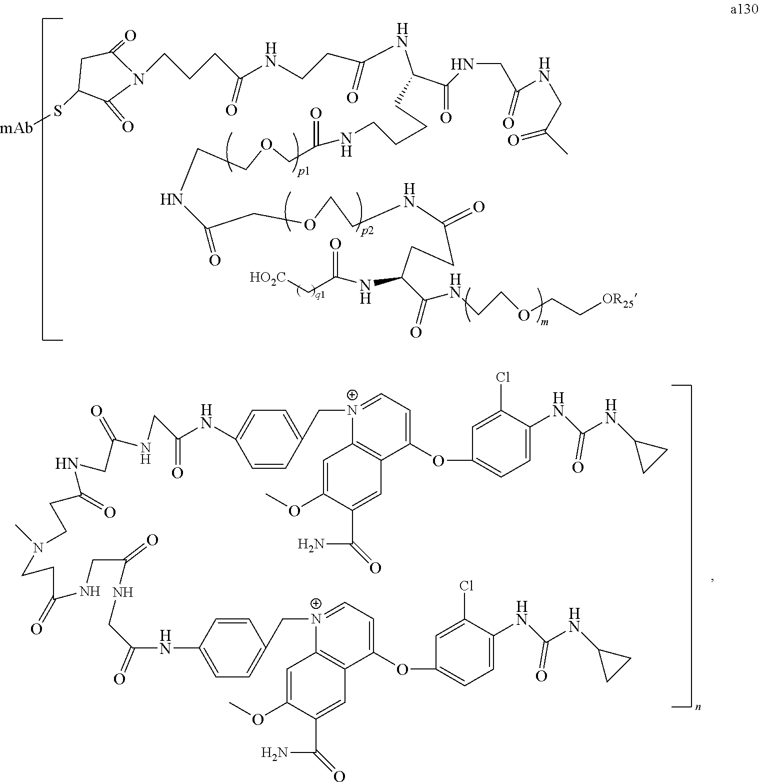

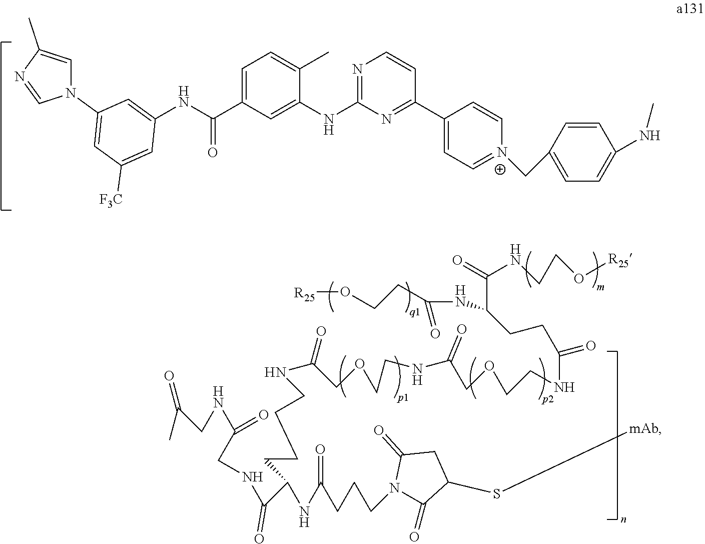

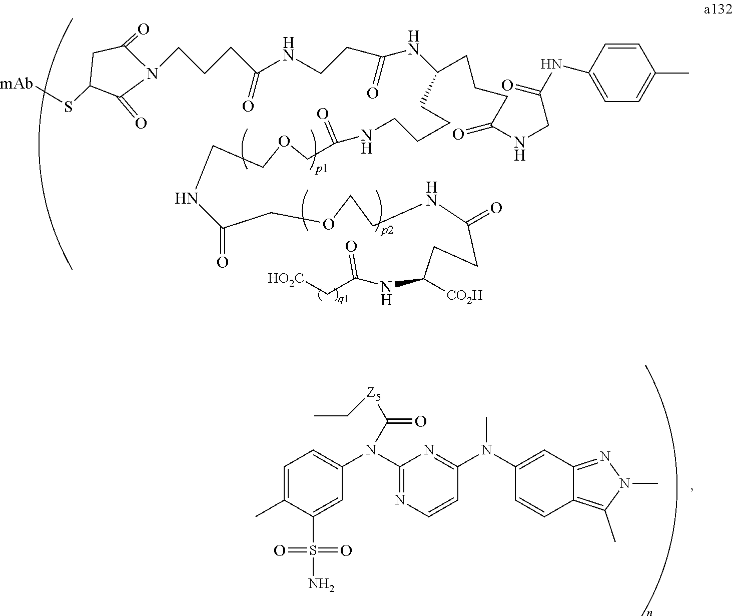

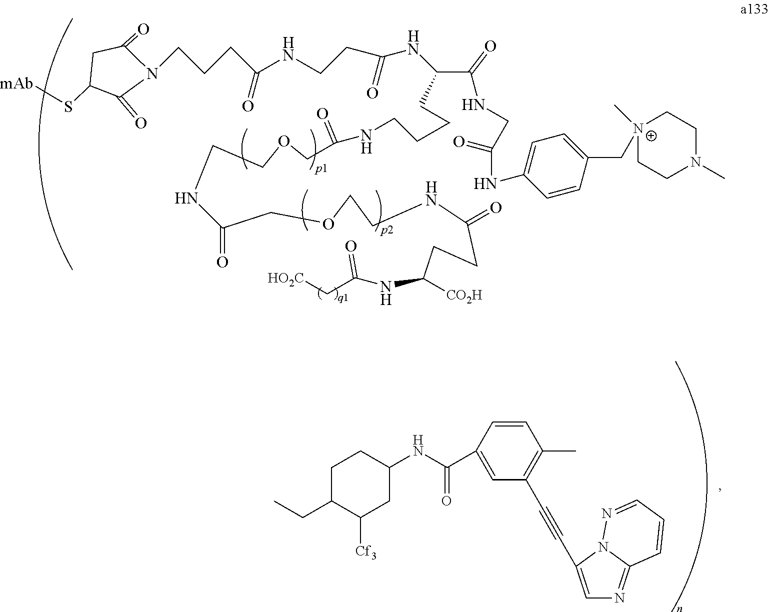

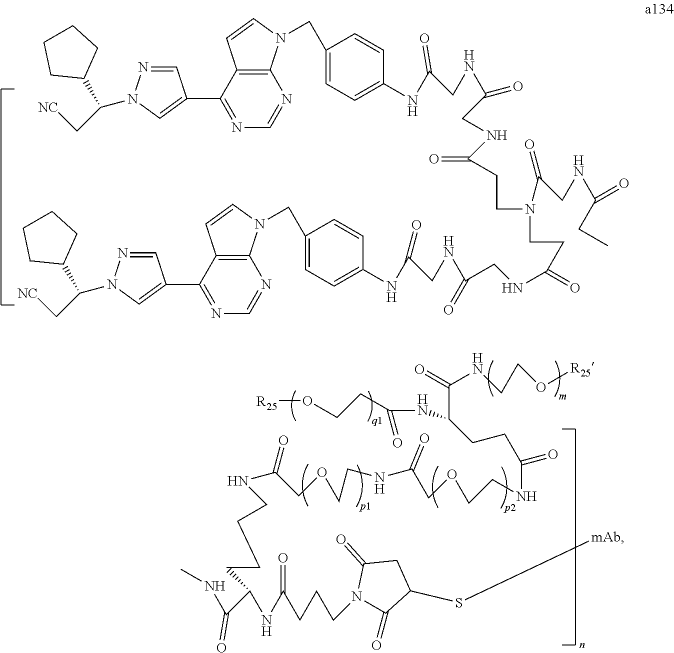

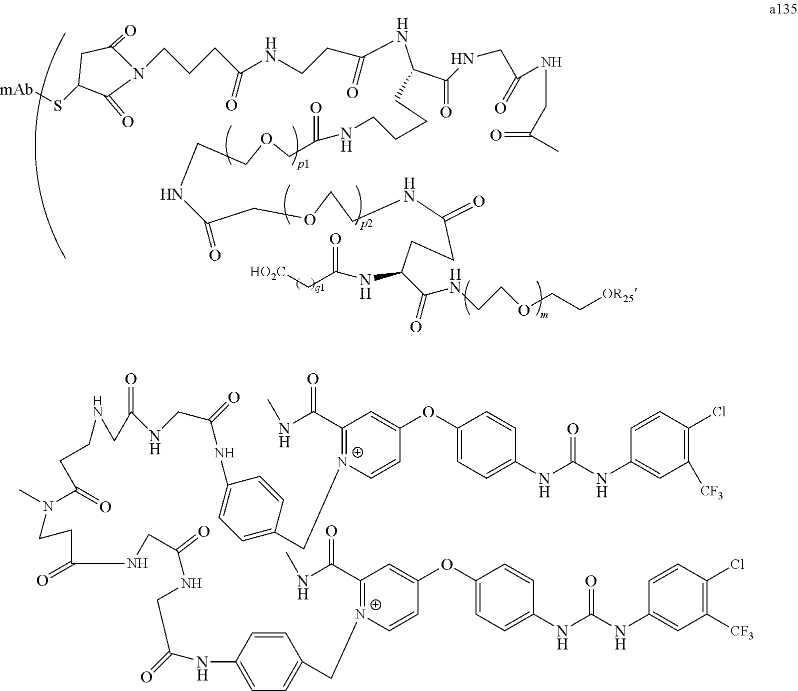

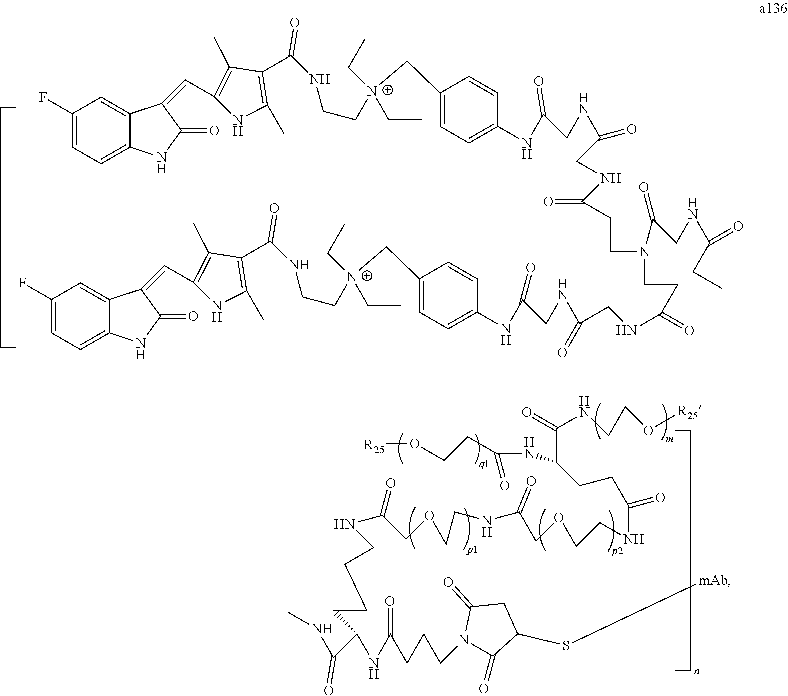

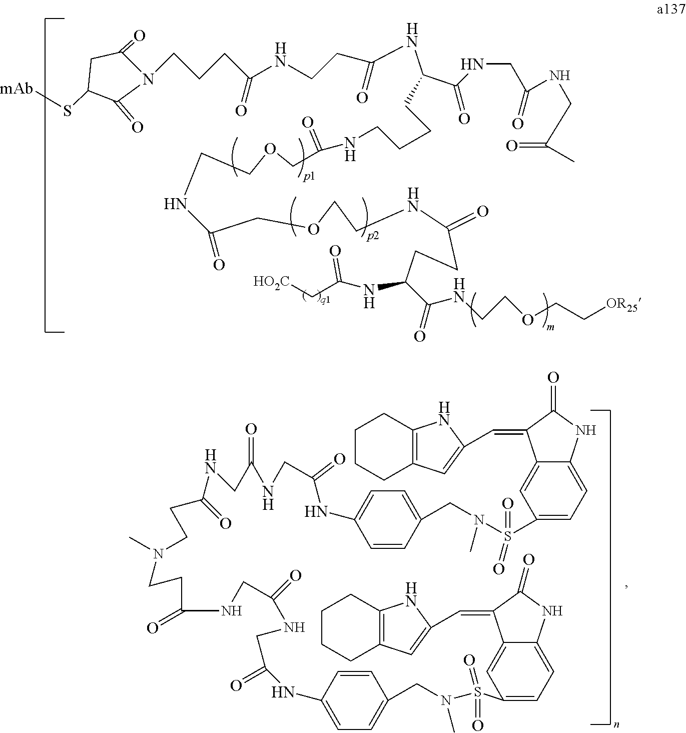

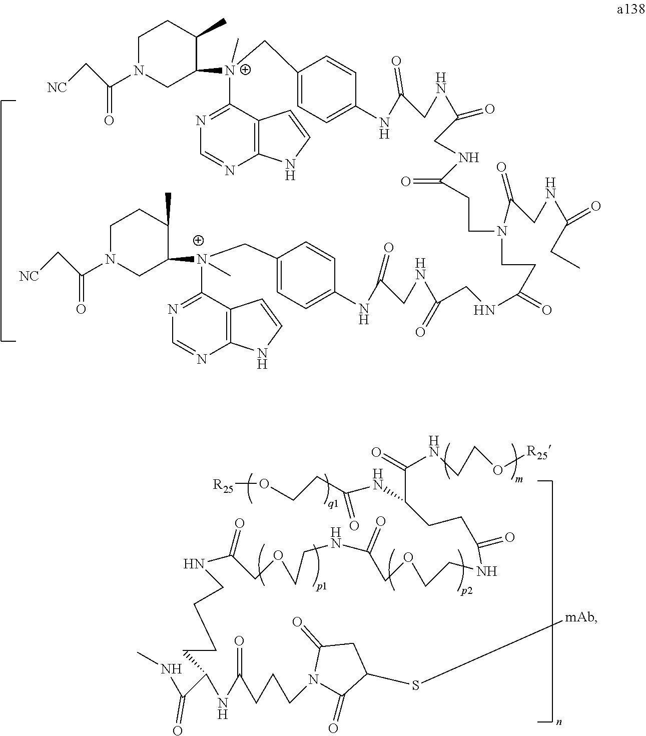

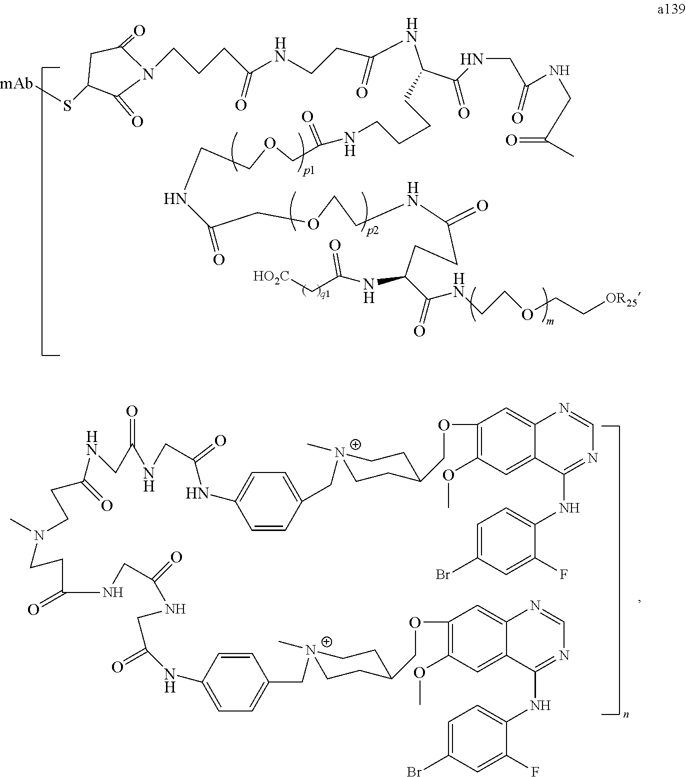

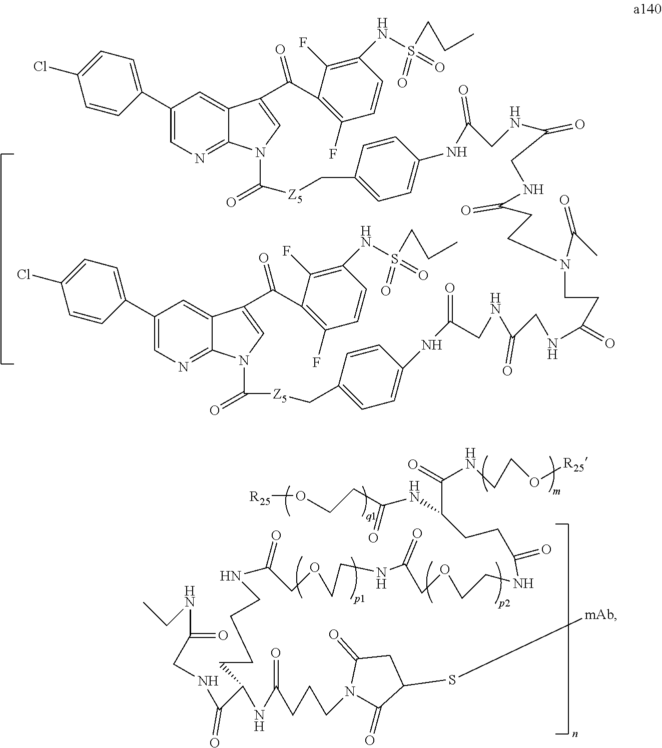

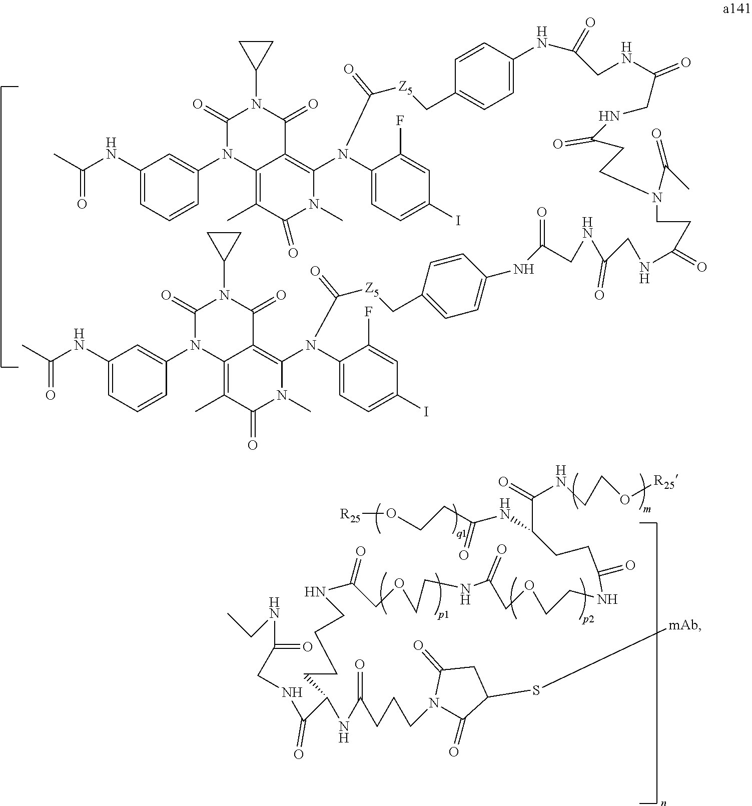

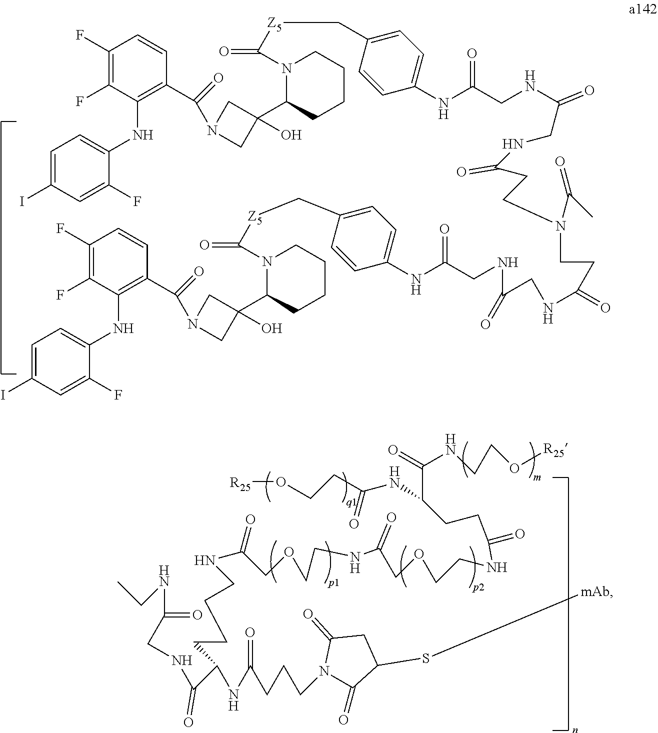

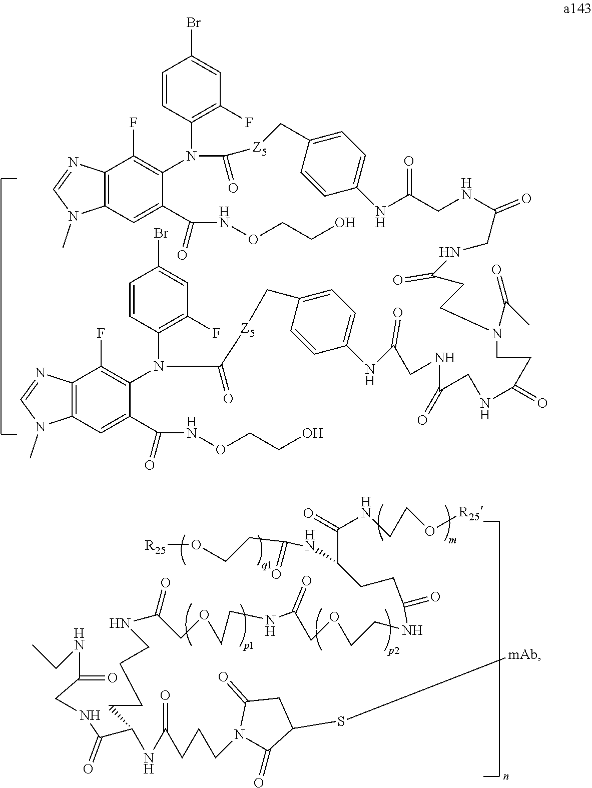

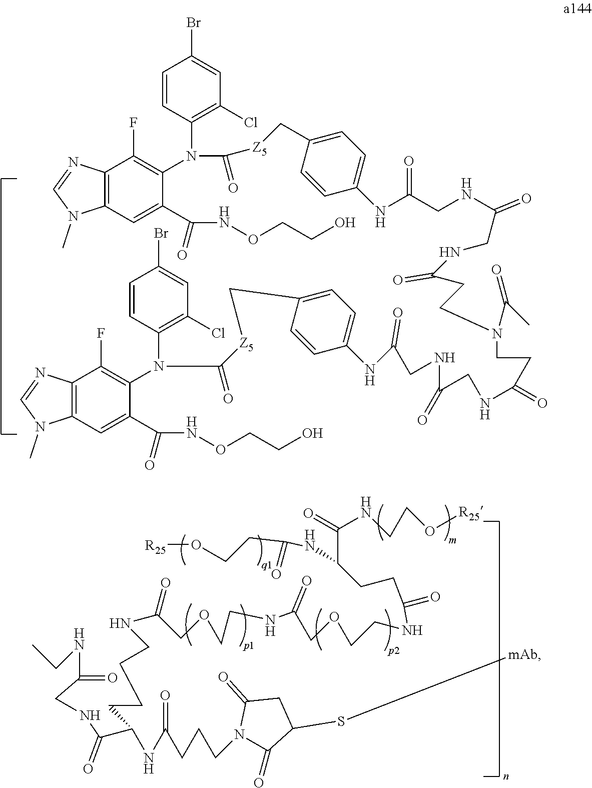

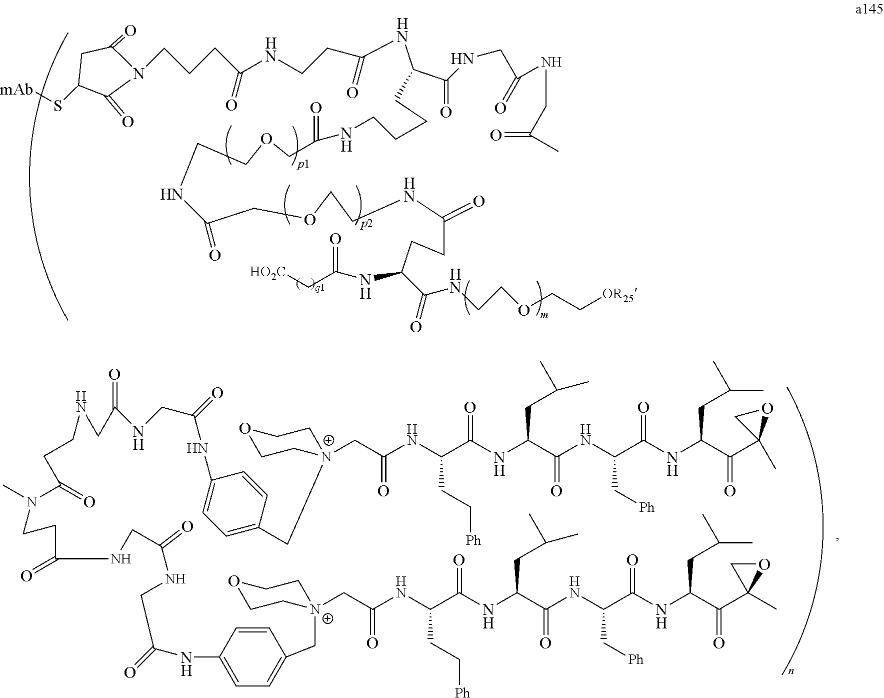

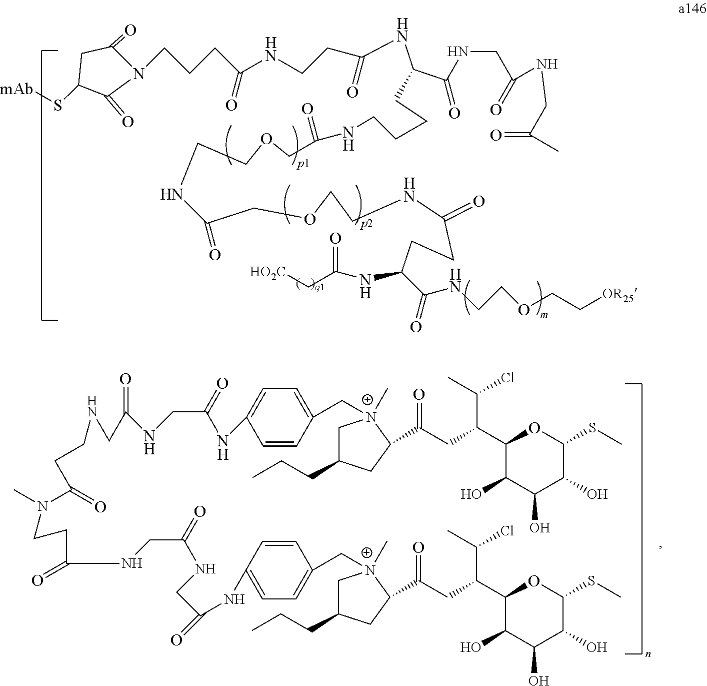

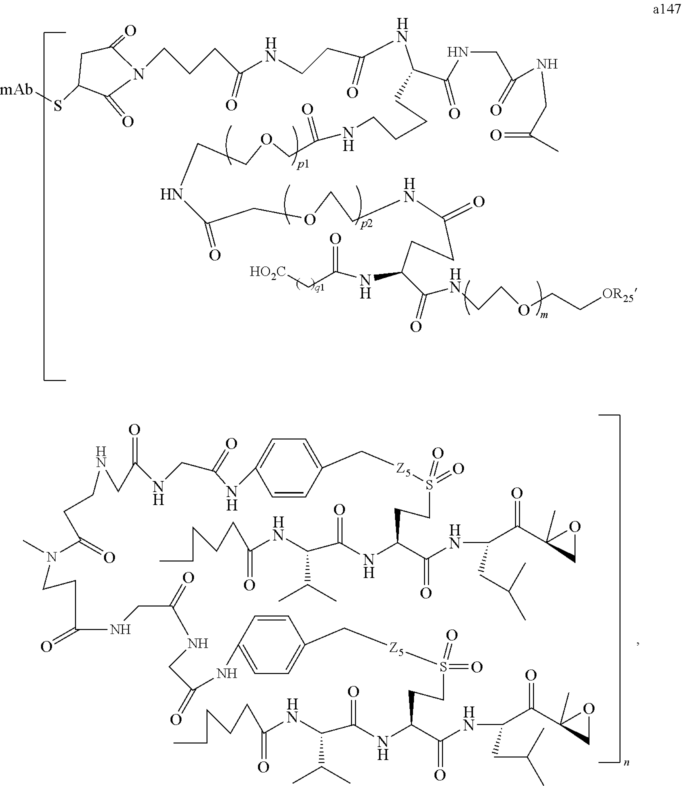

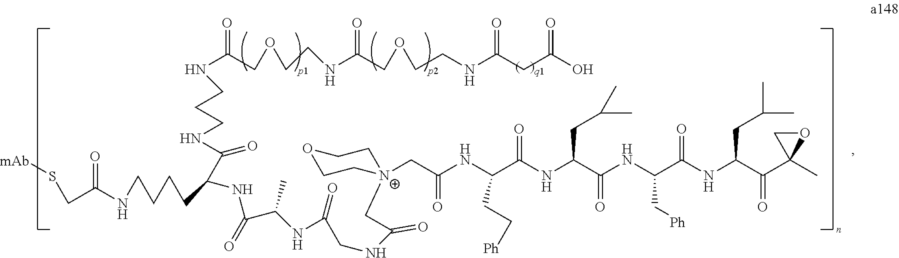

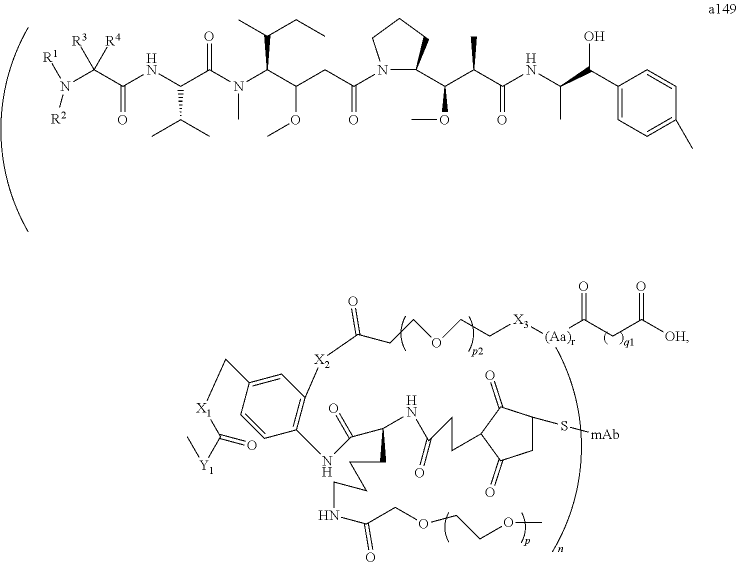

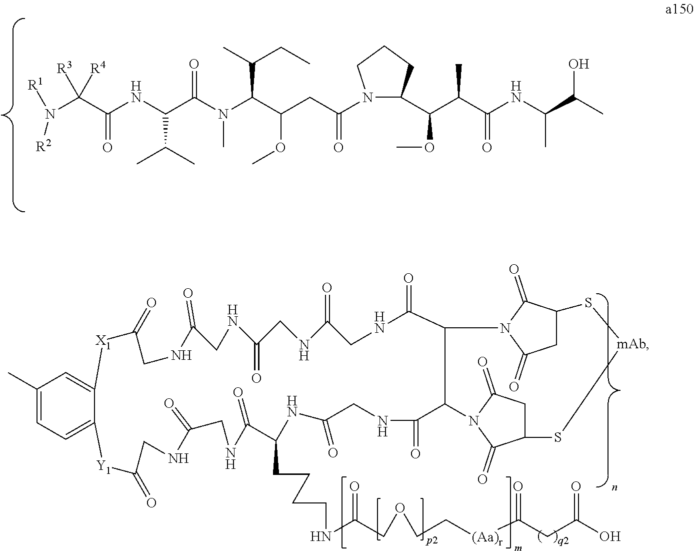

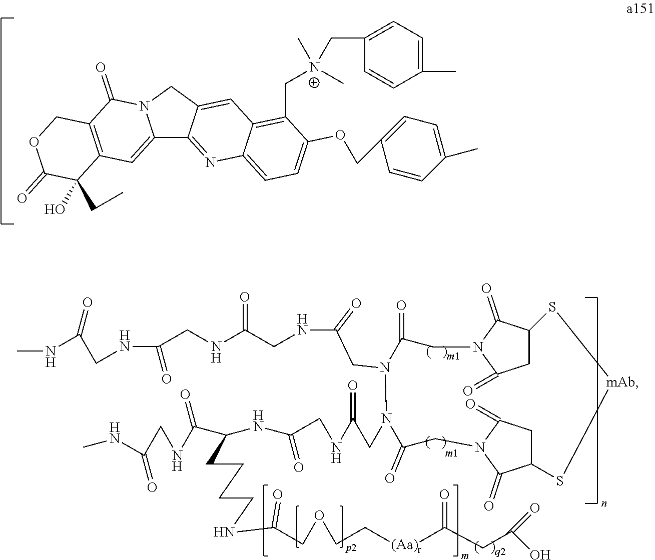

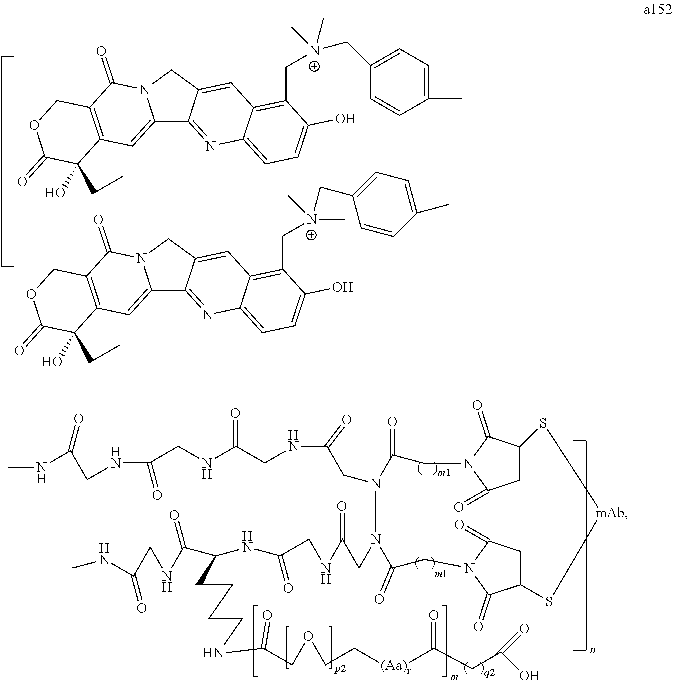

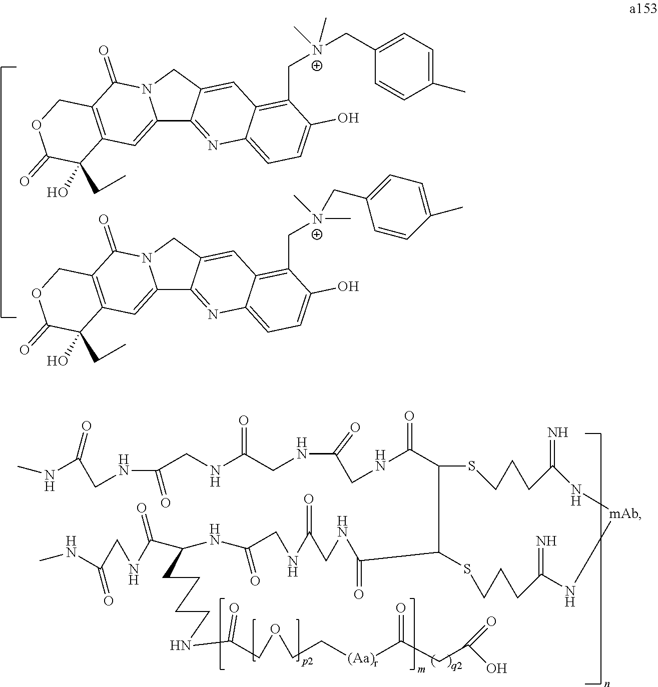

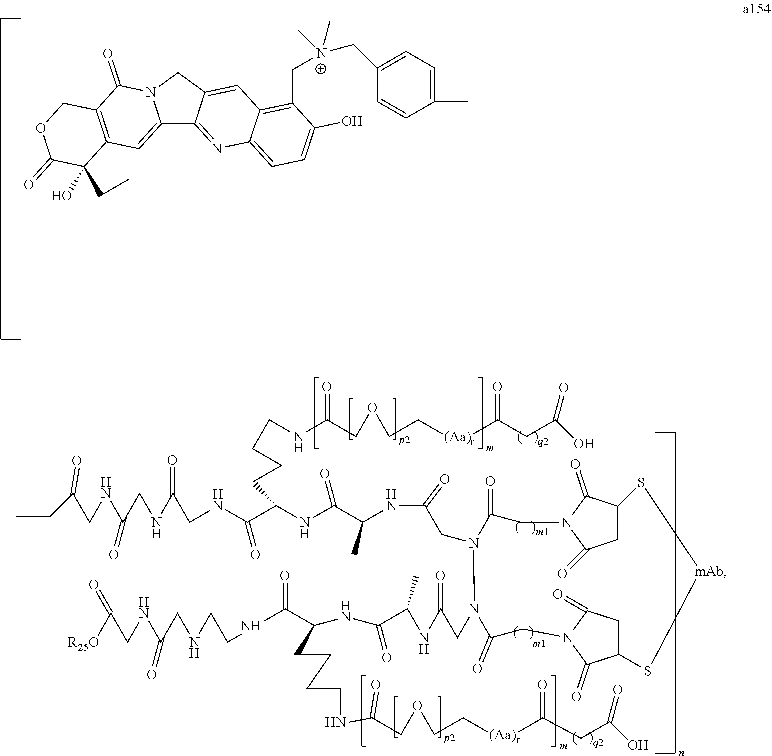

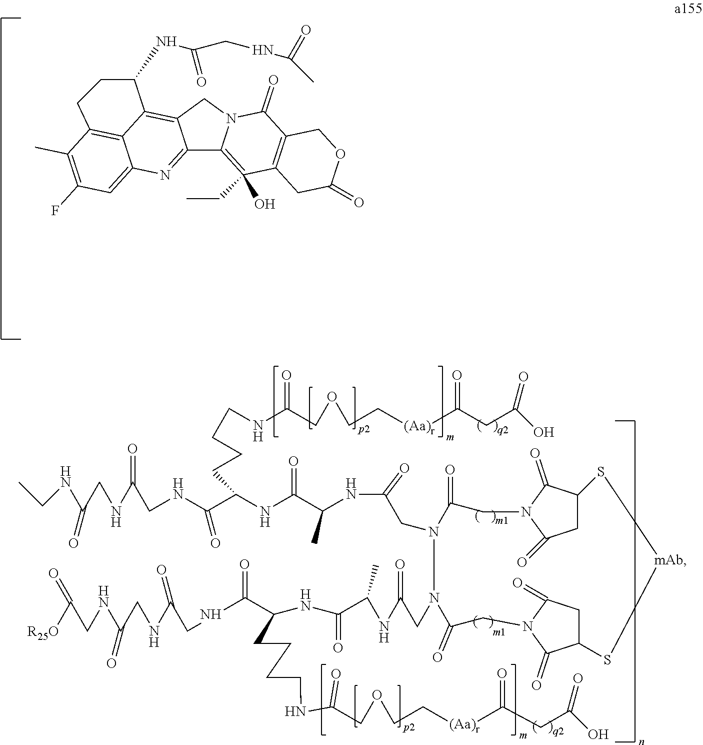

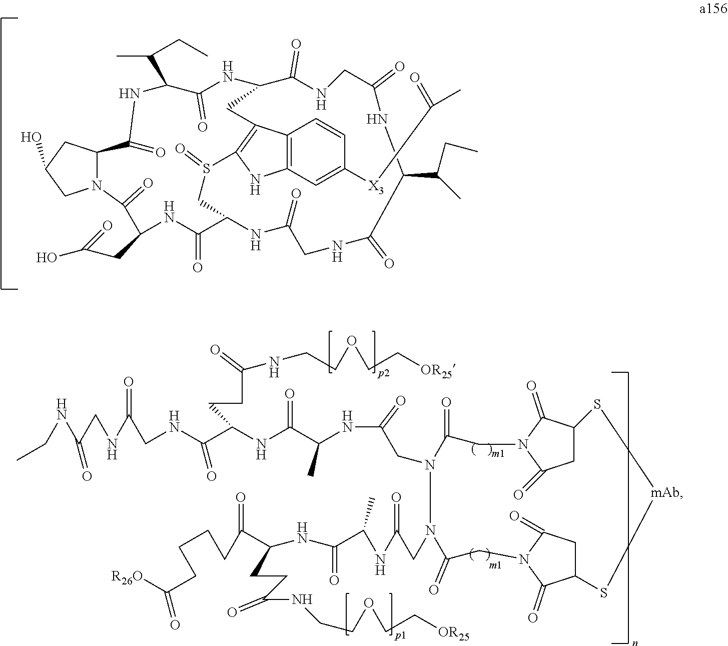

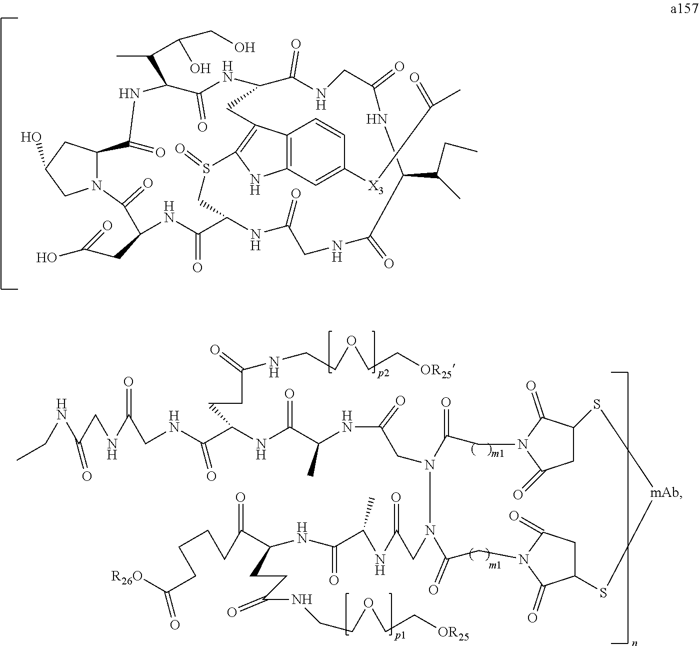

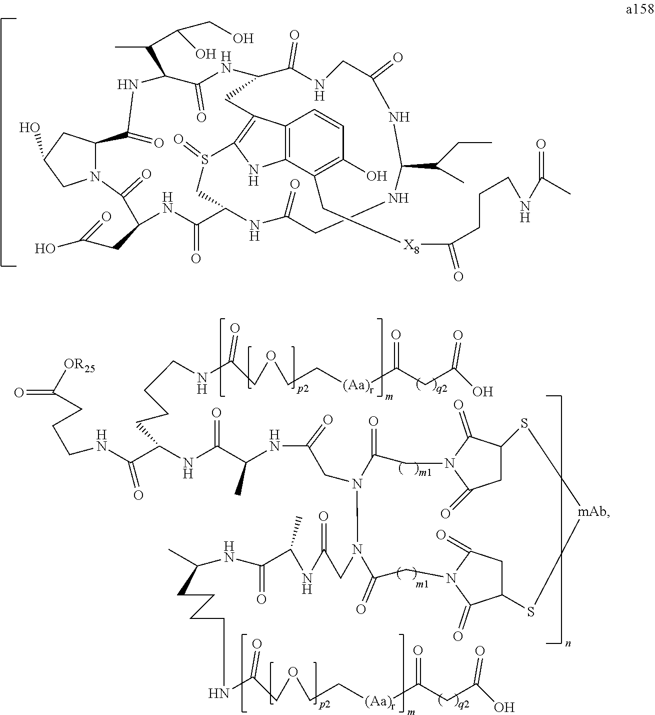

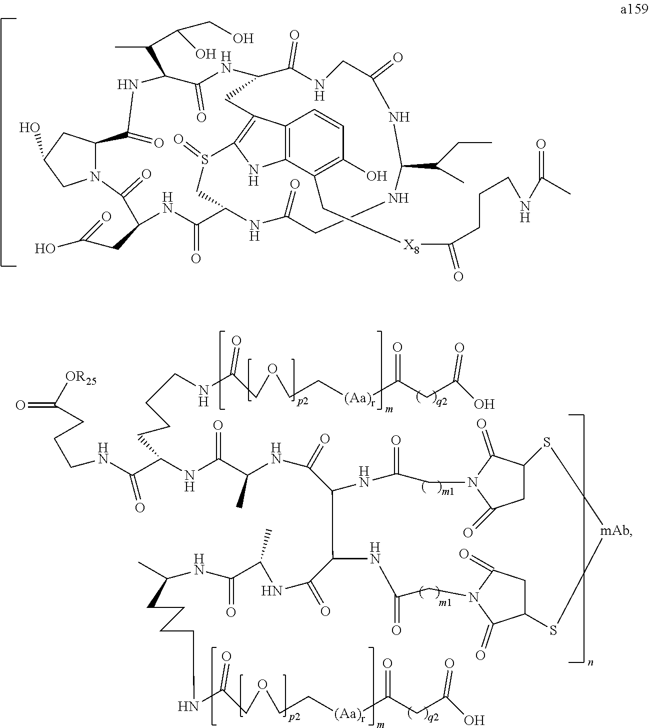

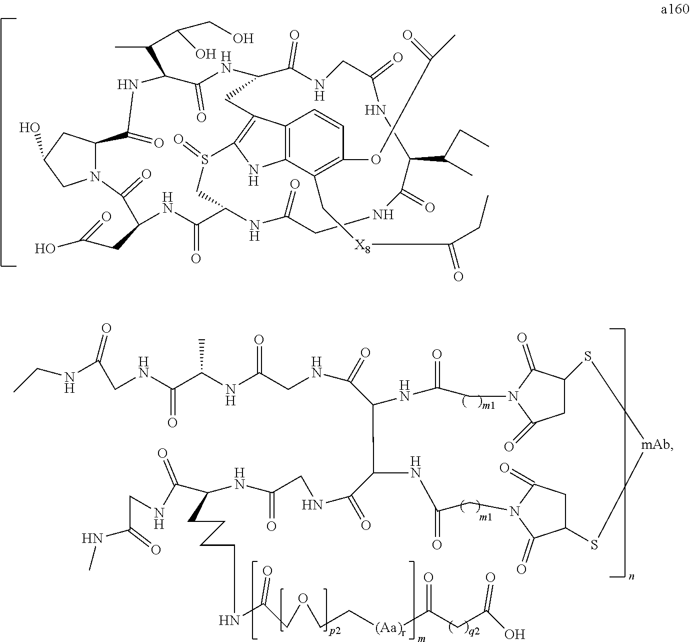

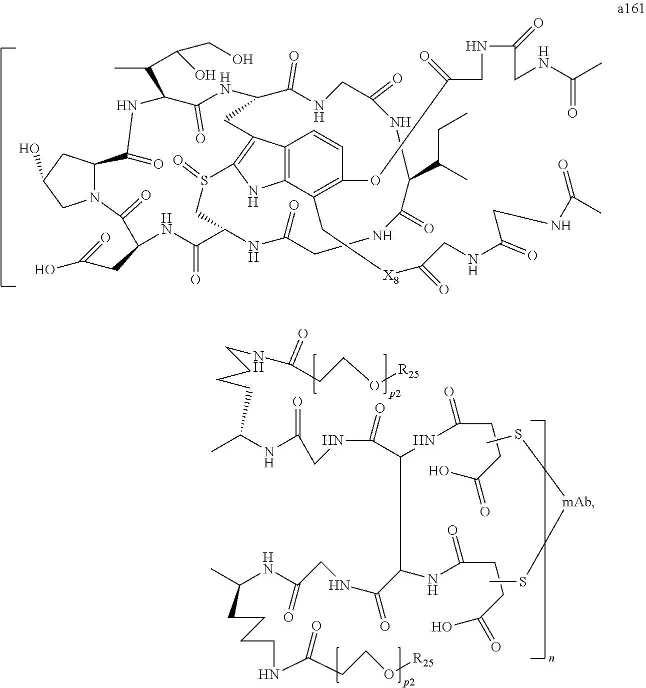

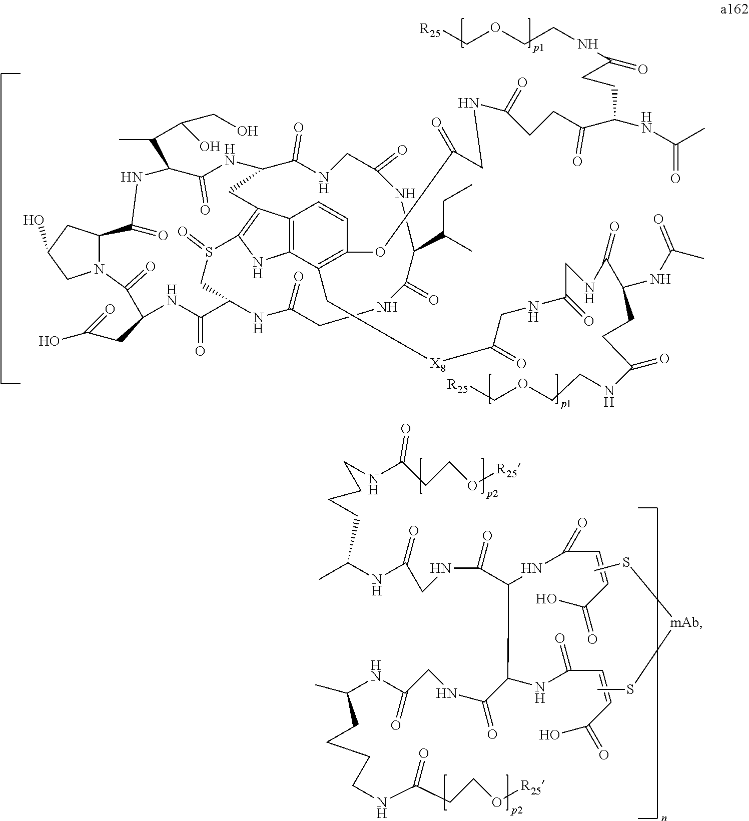

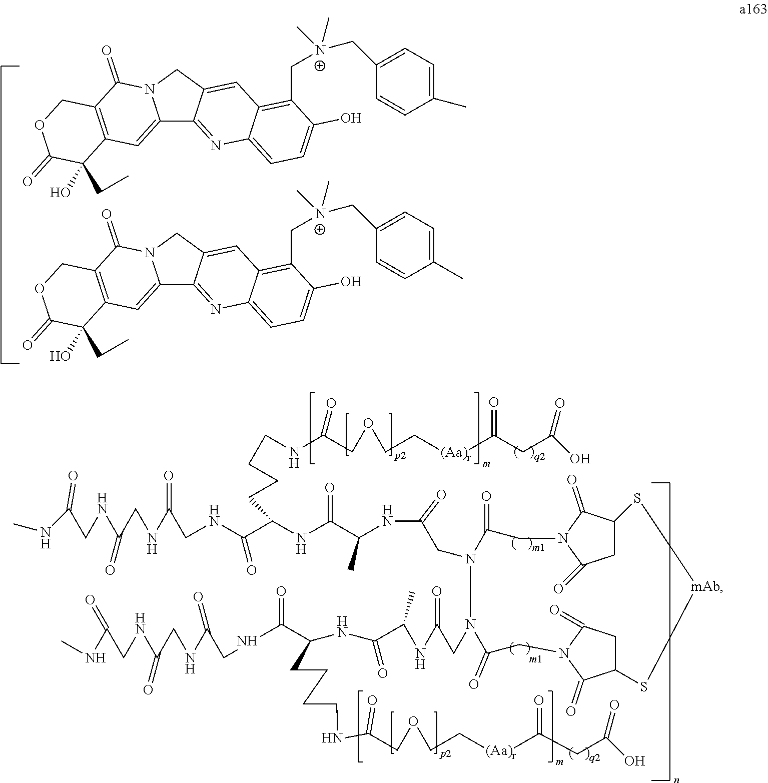

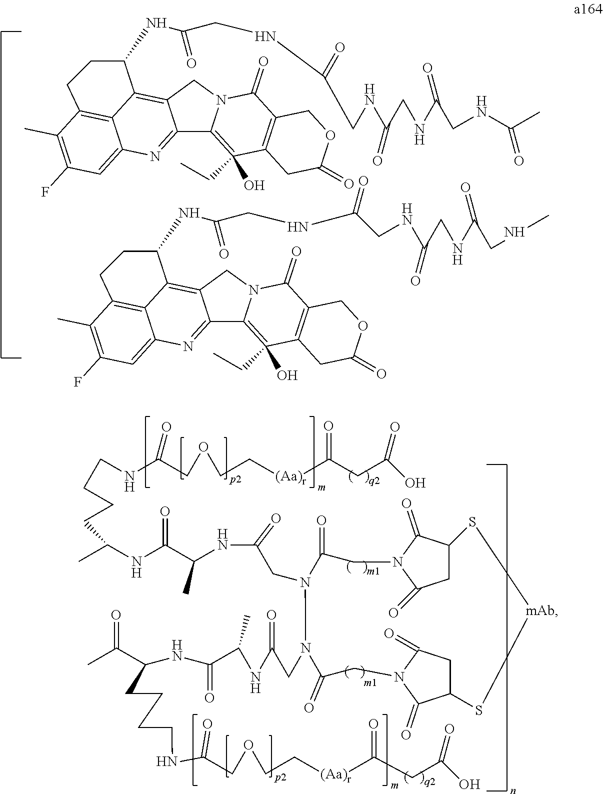

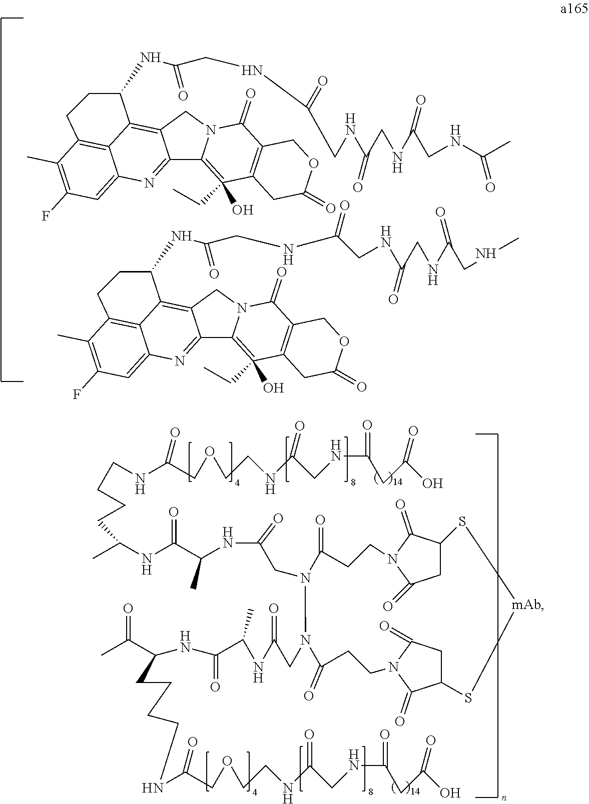

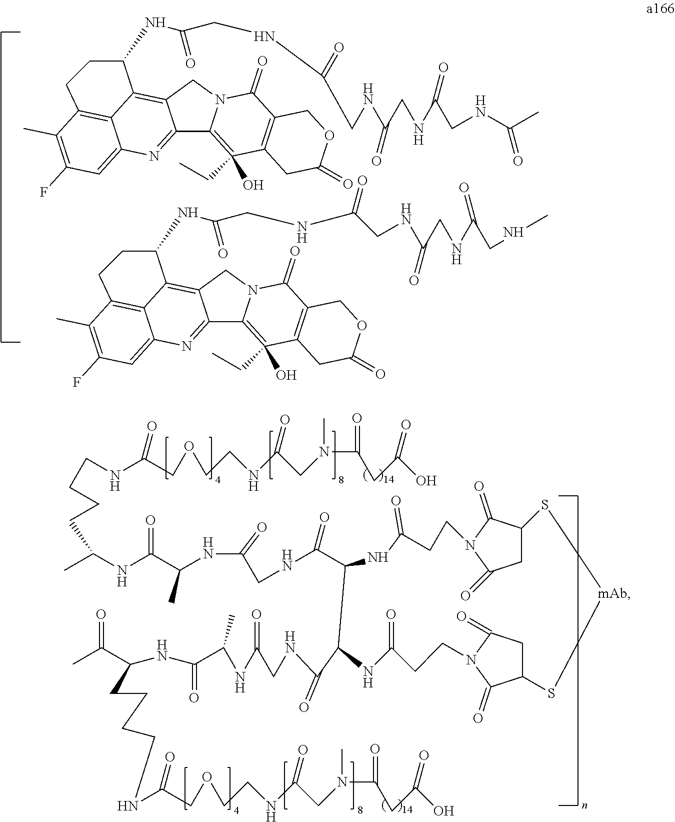

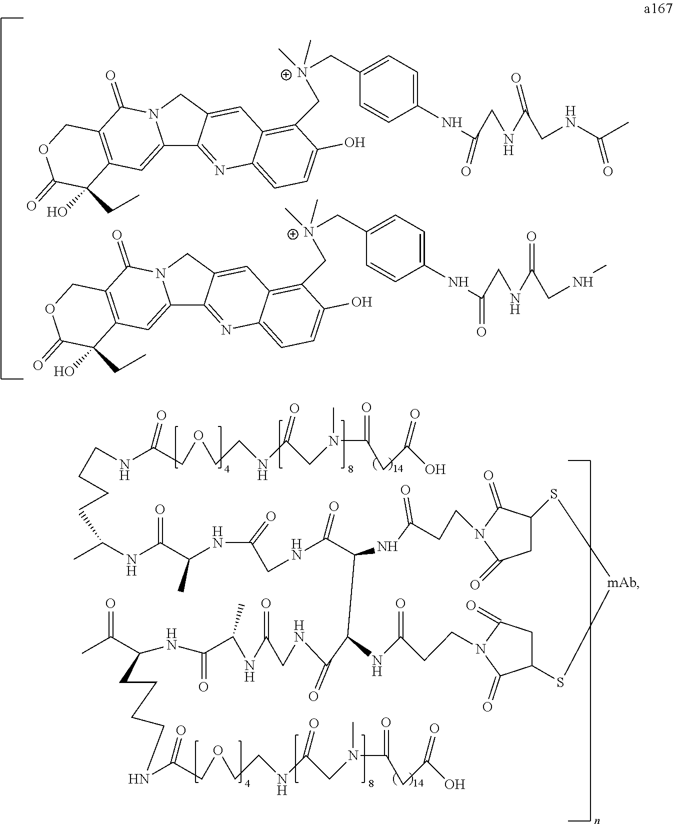

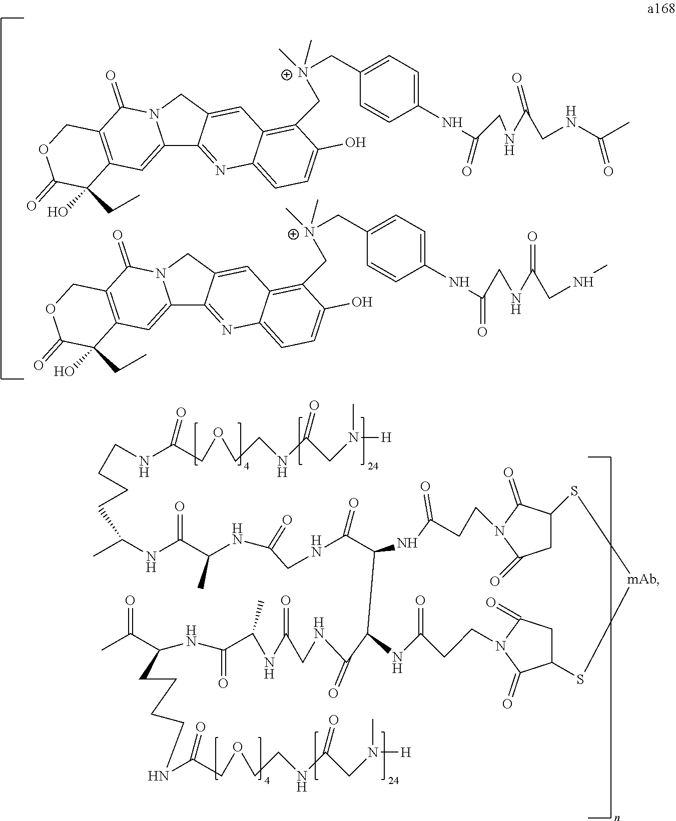

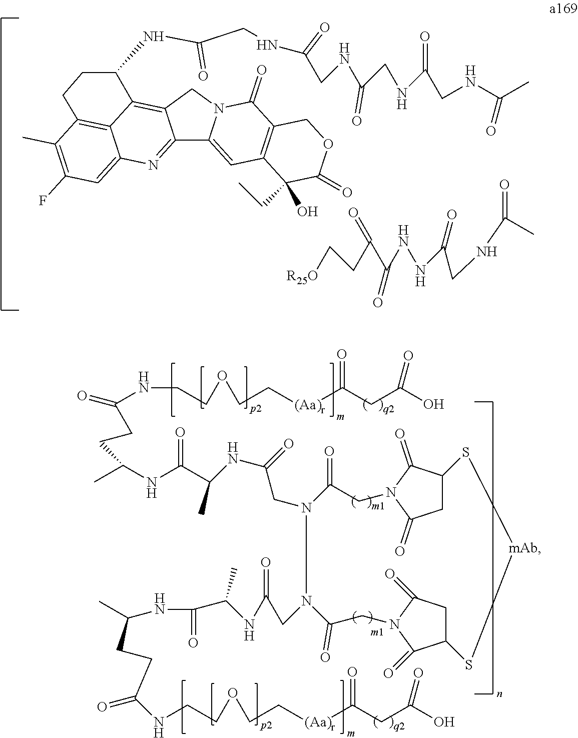

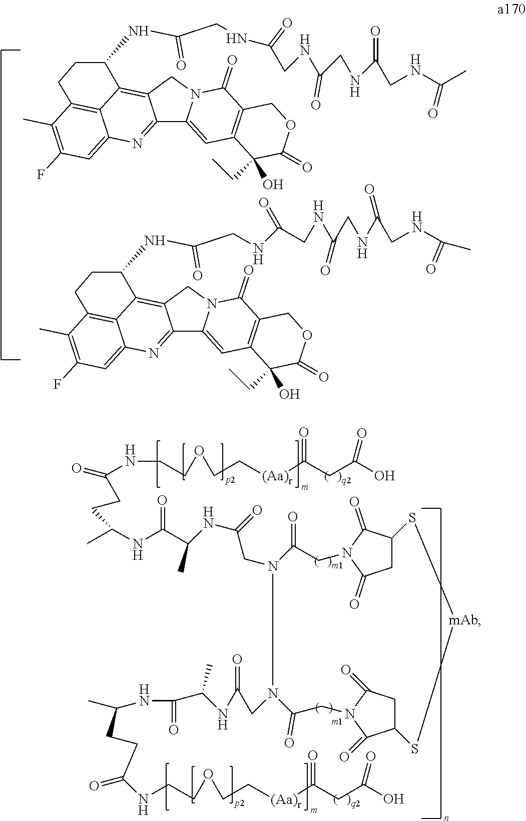

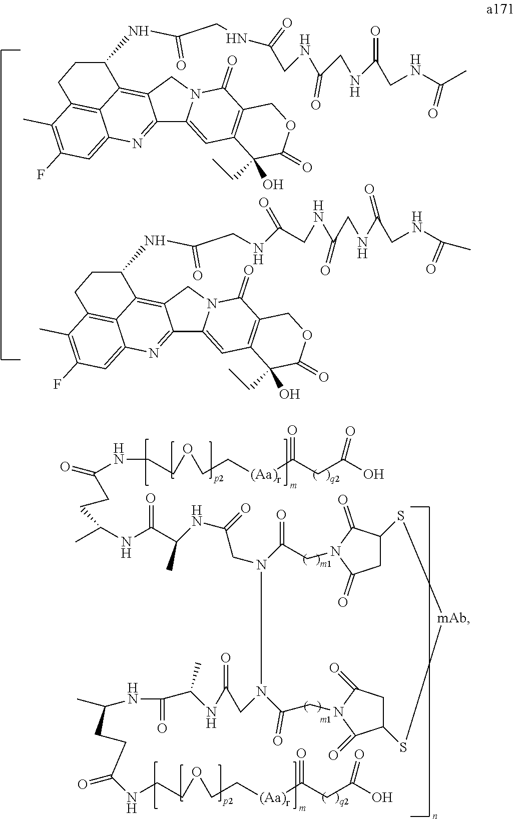

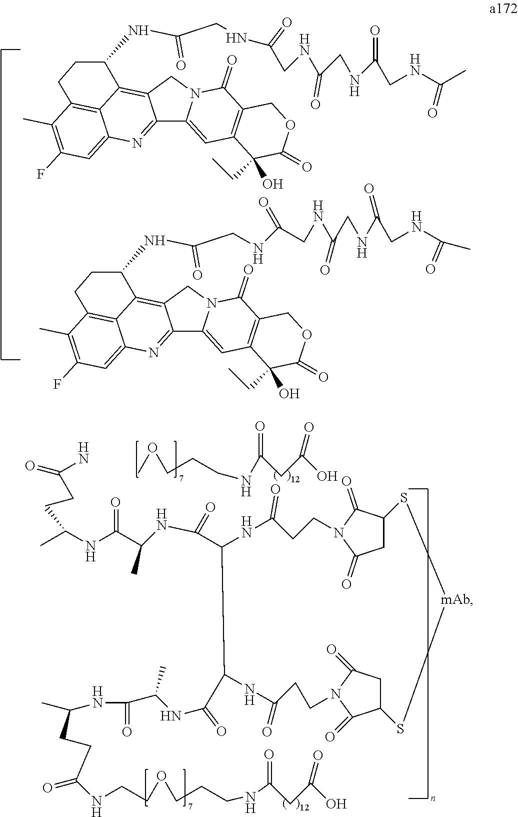

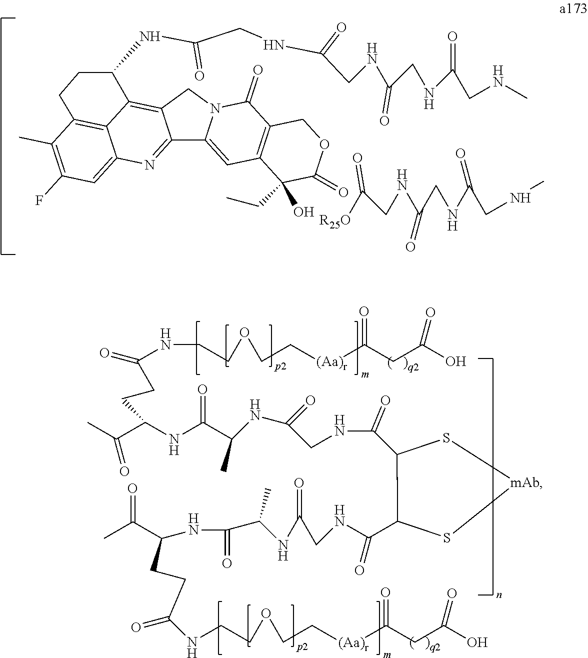

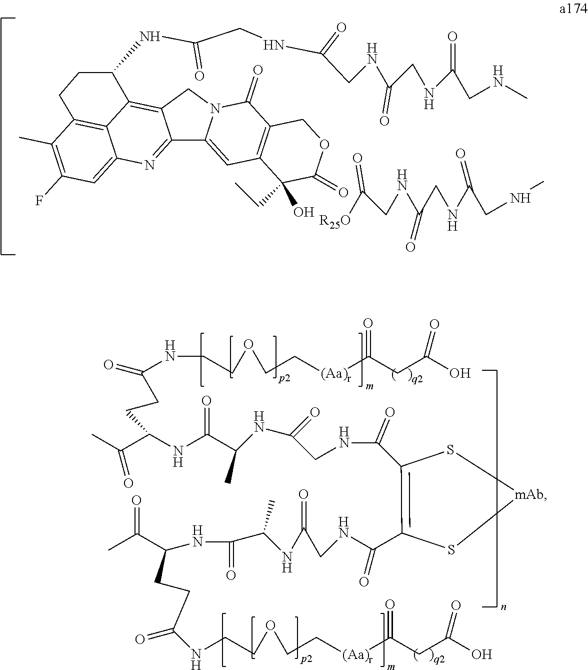

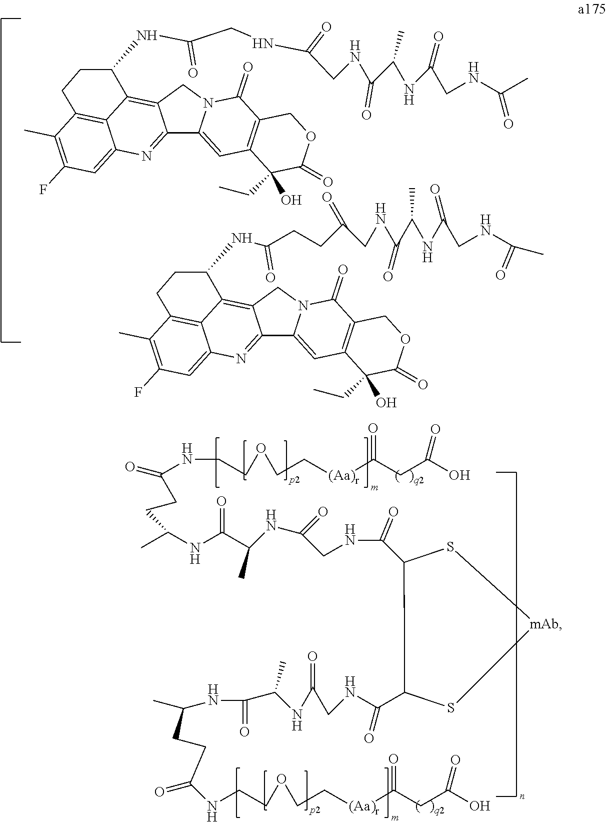

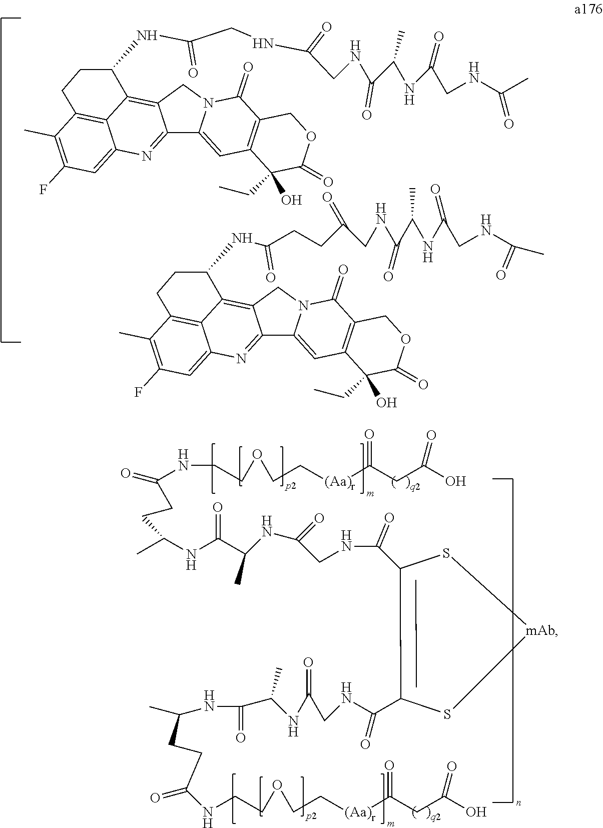

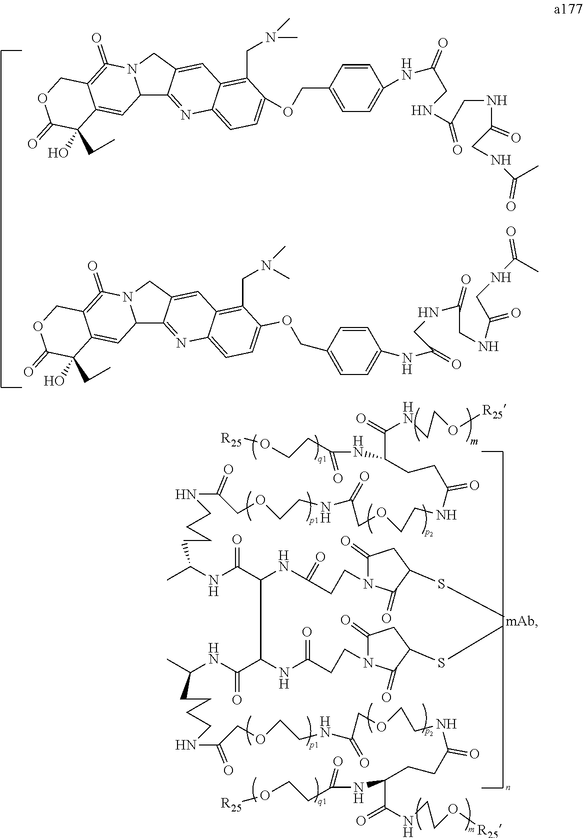

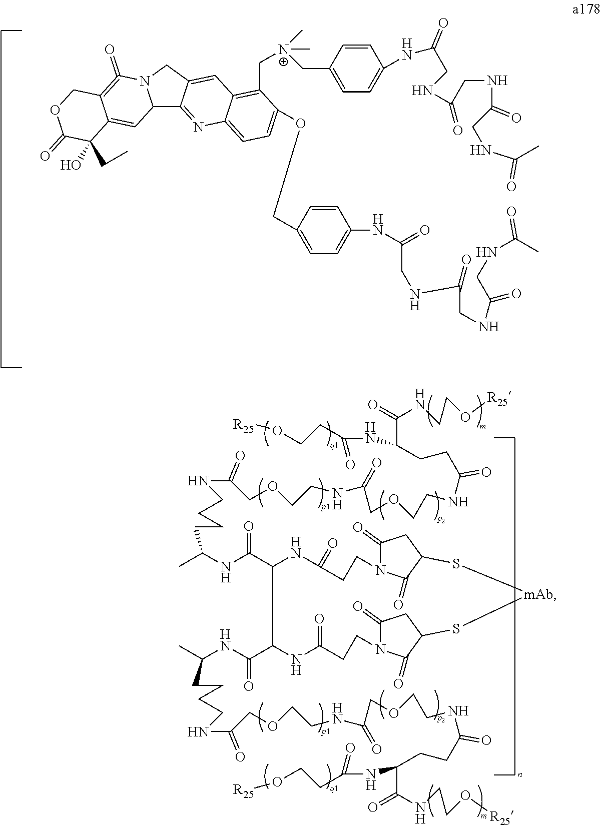

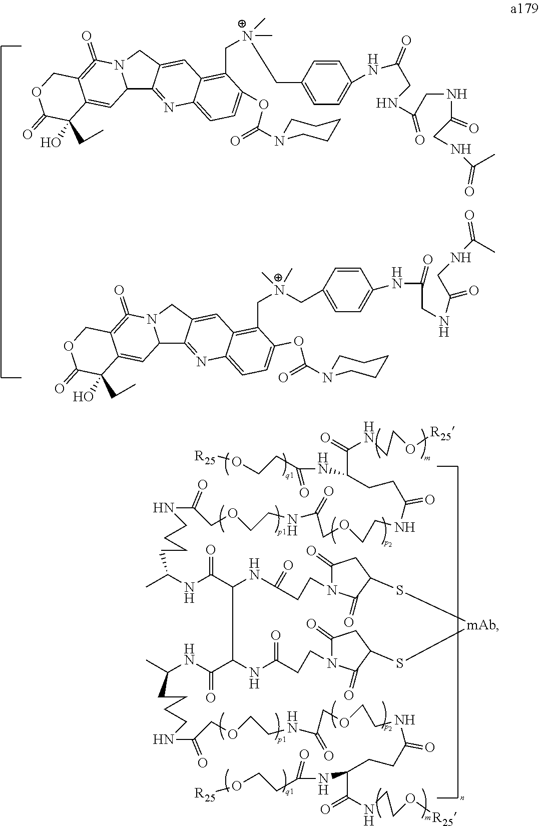

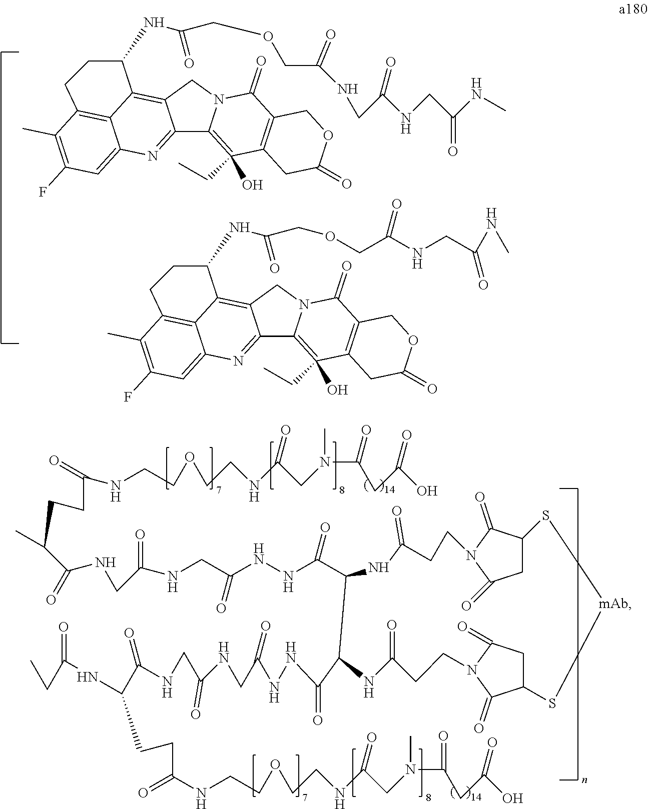

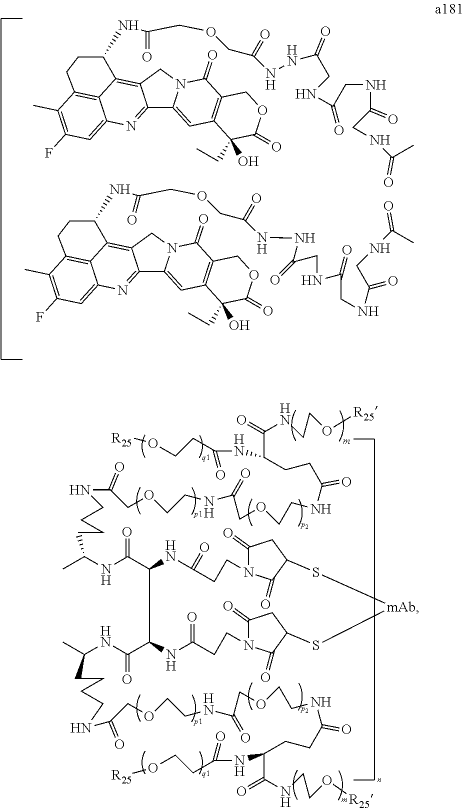

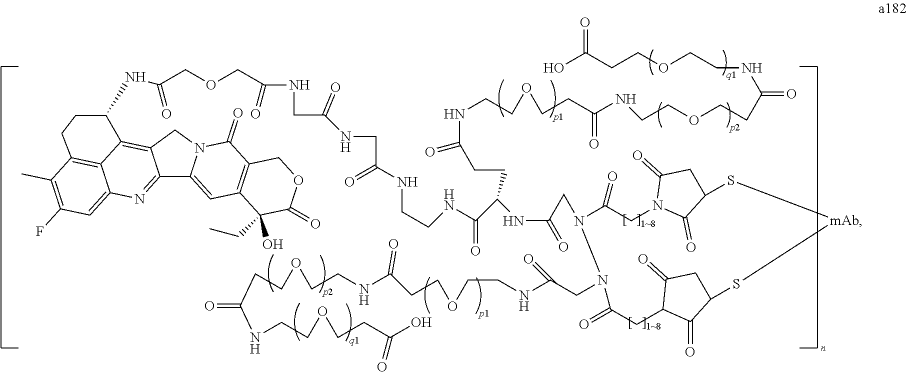

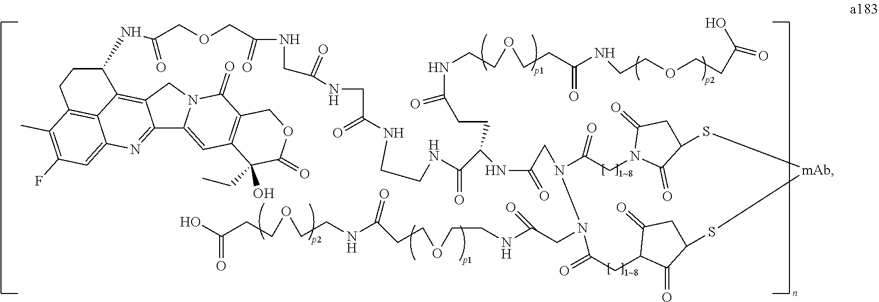

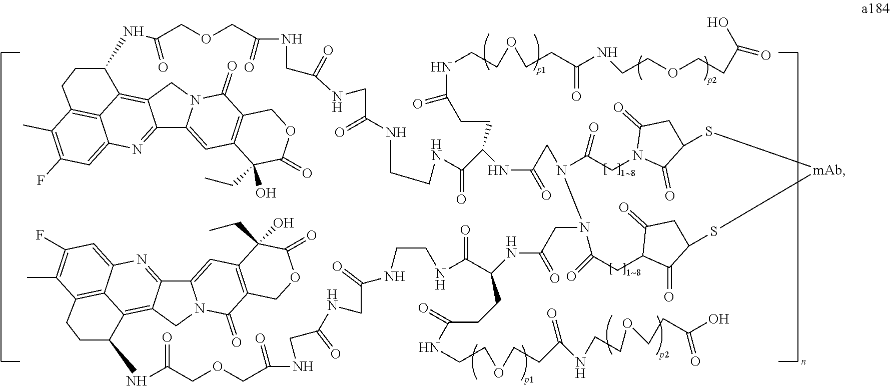

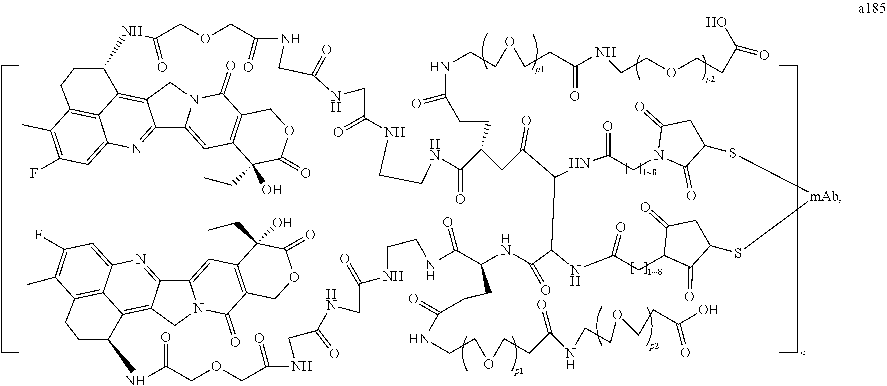

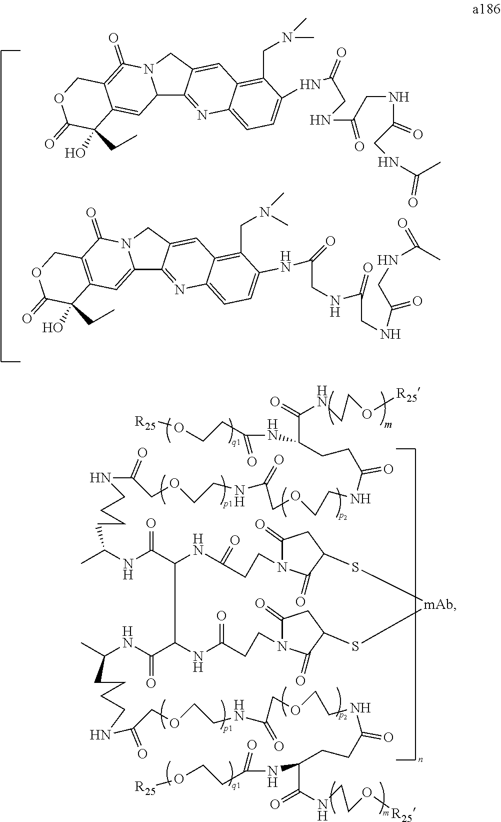

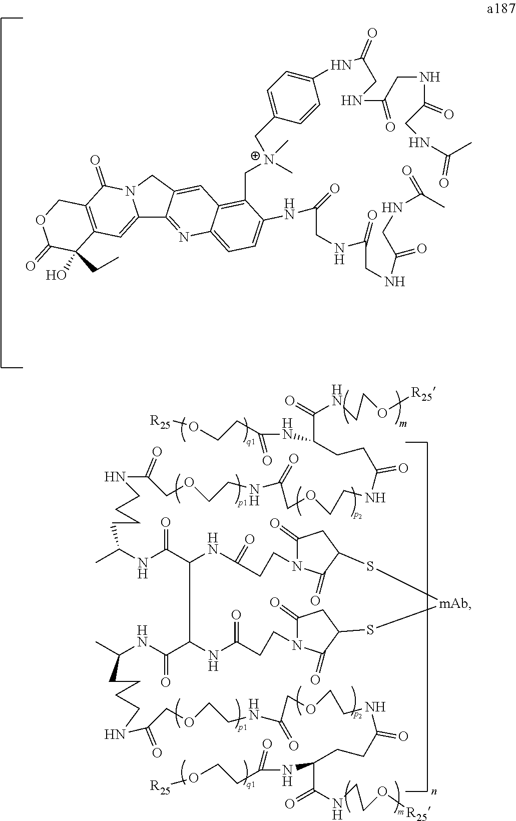

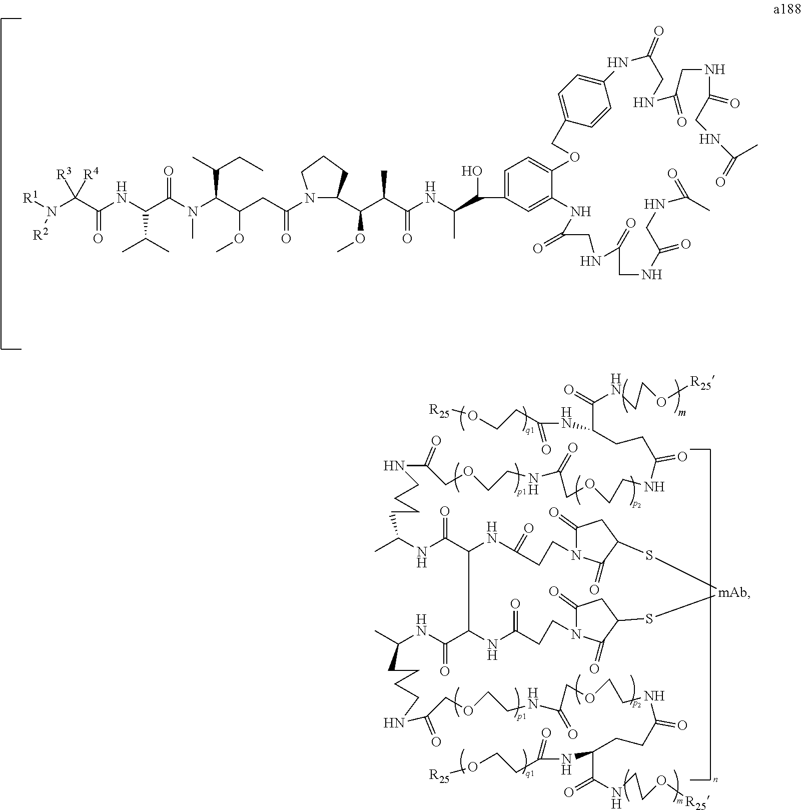

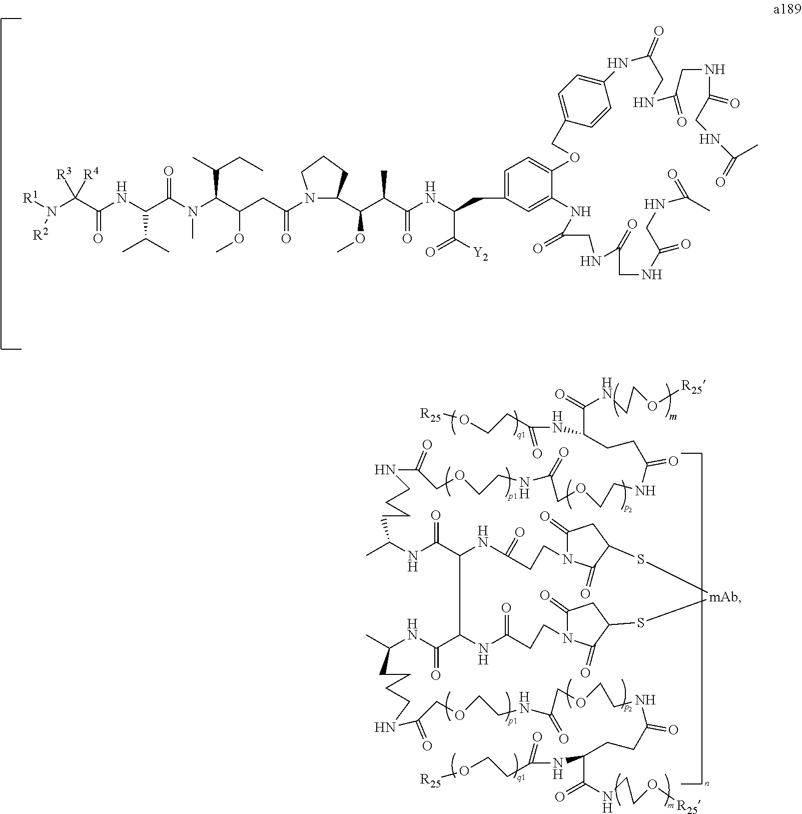

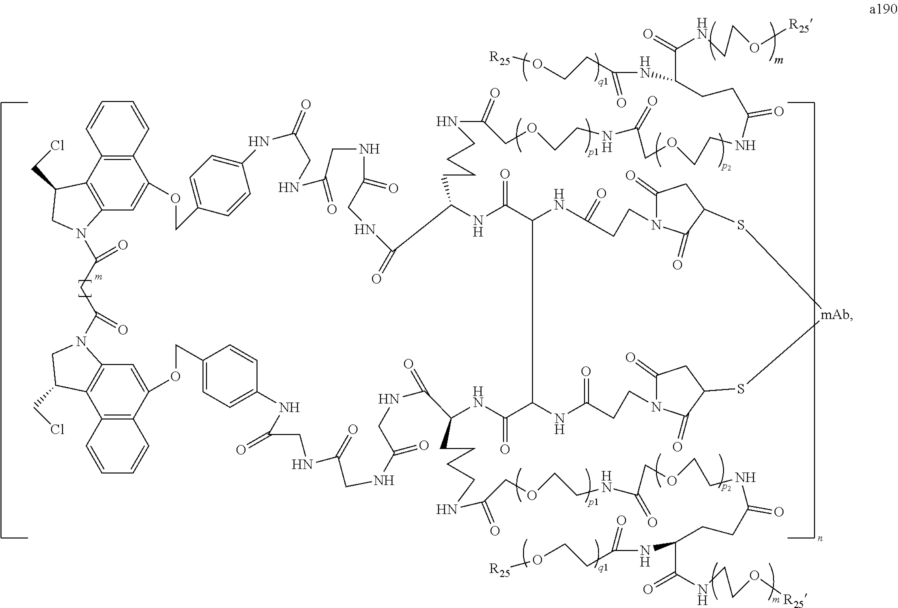

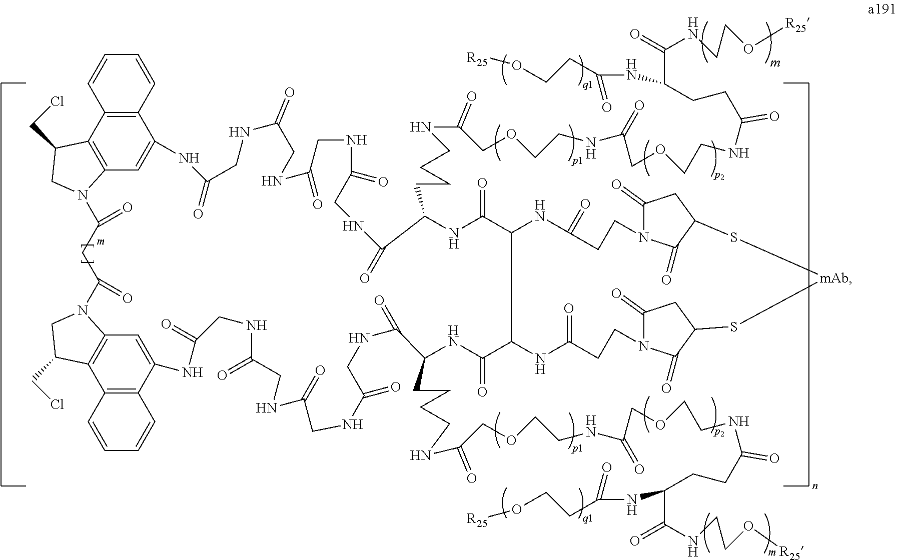

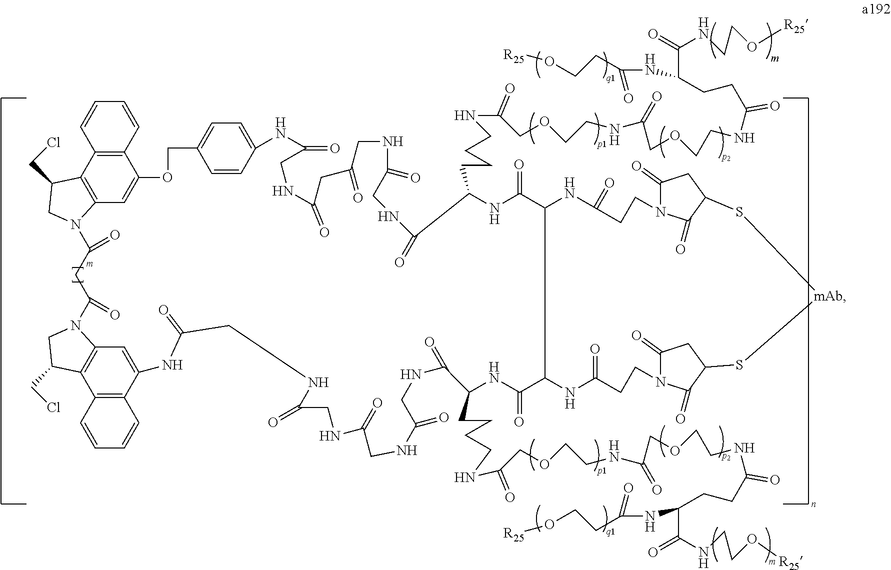

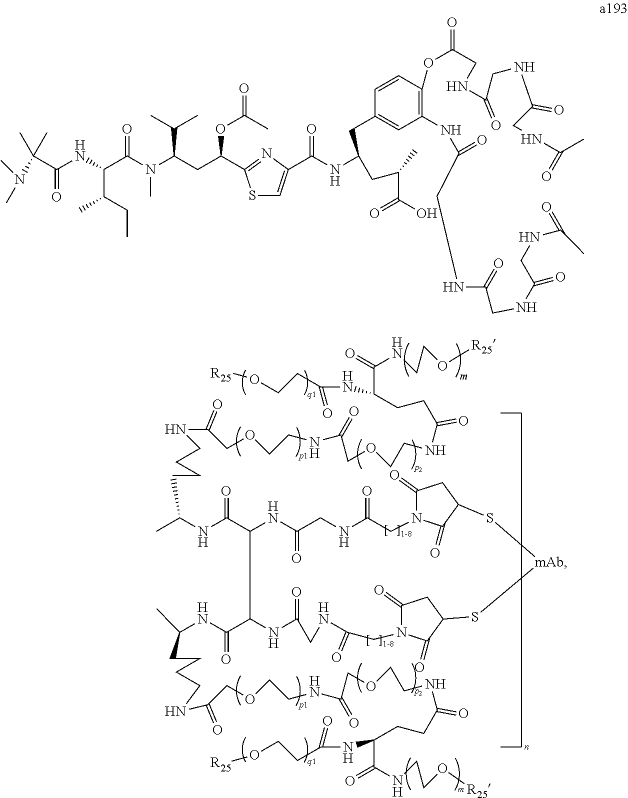

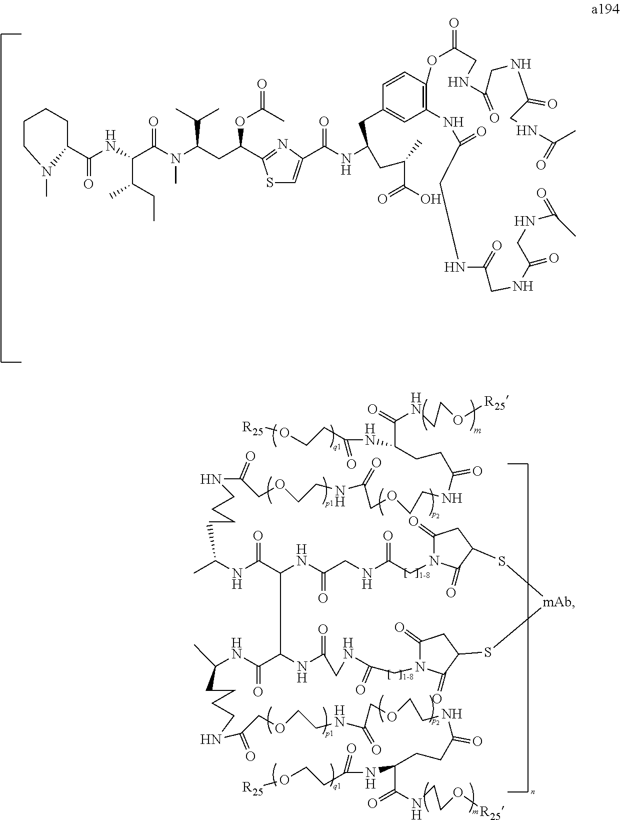

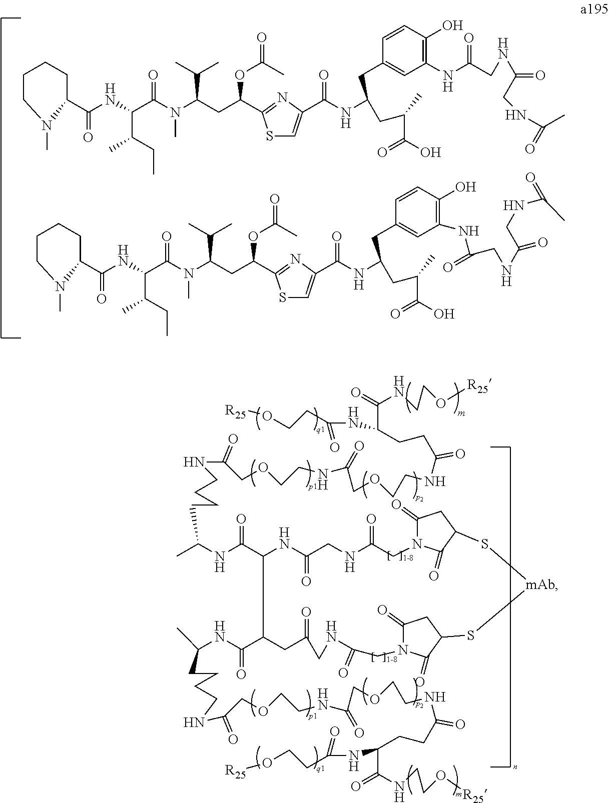

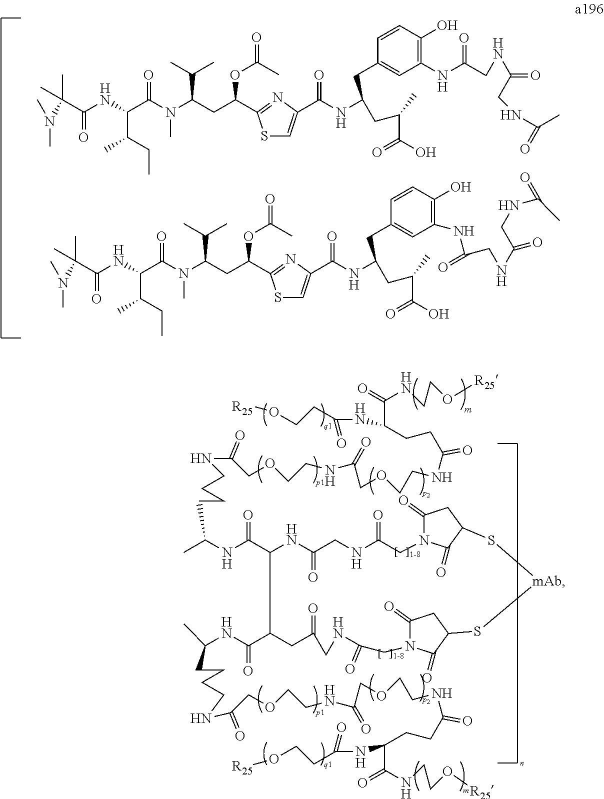

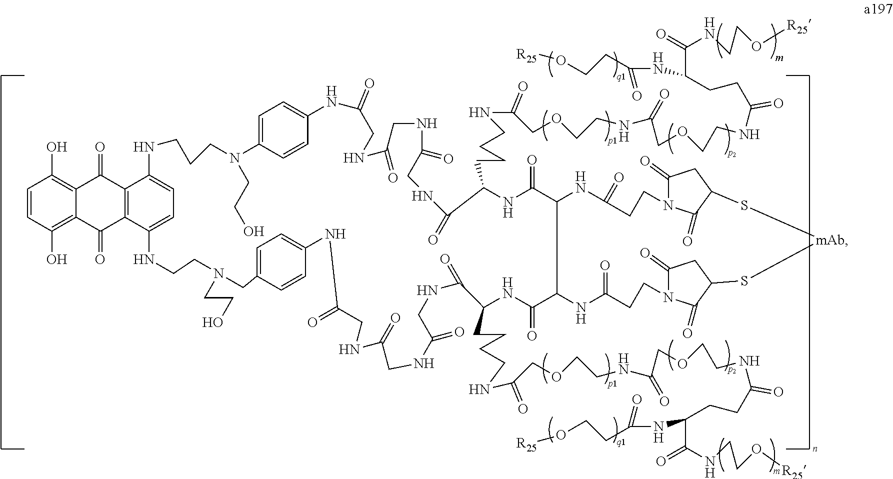

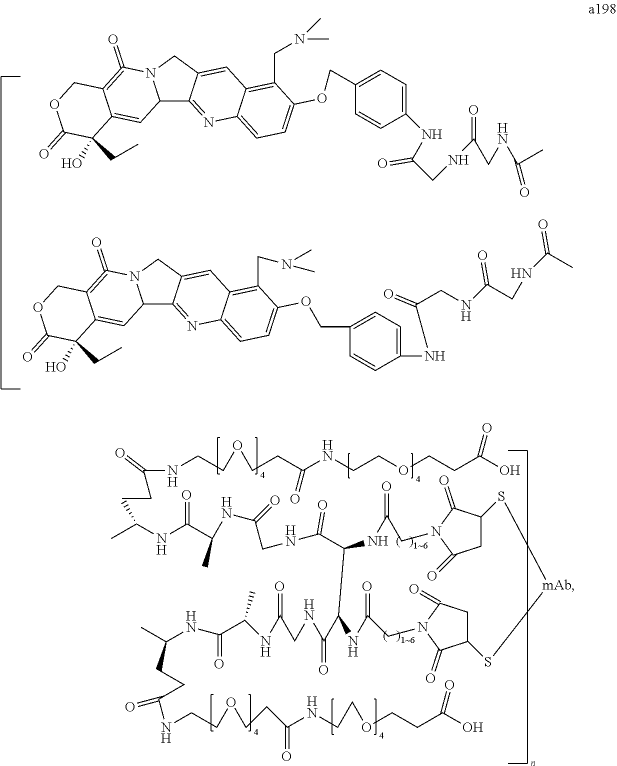

or one or more isotope of chemical elements, pharmaceutically acceptable salts, hydrates, or hydrated salts; or the polymorphic crystalline structures of these compounds; or the optical isomers, racemates, diastereomers or enantiomers; wherein X₈ is O, S, NH, NHNH, NHR₁₂, SR₁₂, SSR₁₂, SSCH(CH₃)R₁₂, SSC(CH₃)₂R₁₂, or R₁₂; R¹, R², R³, R⁴, R⁵, R₄, R₅, R₇, R₈, R₉, R₁₀, X₁, X₂, X₃, X₄, X₅, X₆, Y₁, Y₂, Y₃, Y₅, R₁₂, R_{12′}, R₁₃, R₁₃′, R₂₅, R₂₅′, p₁, p₂, q₁, q₂, m, m₁, n, and mAb are described the same above; Aa is natural or unnatural amino acid; r is 0-12; (Aa)ᵣ is a peptide containing the same or different sequence of amino acids when r>2; r=0 means (Aa)r absent.

In another aspect of the present invention, the side chain-linkage compound is represented by Formula (IV), (V) and (VI) which can readily react to a cell-binding molecule T, or to a modified cell-binding molecule T to form a conjugate of Formula (I), (II) and (III) respectively:

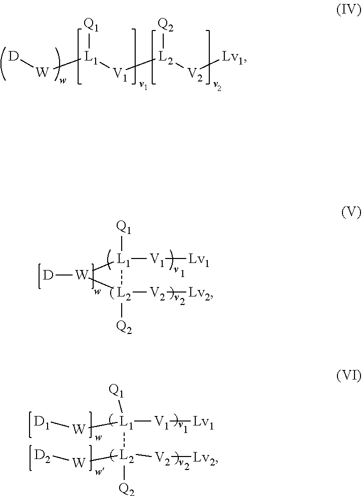

wherein D, D₁, D₂, W, w, w′, L₁, L₂, Q₁, Q₂, V₁, V₂, v₁, v₂, [symbol] and n, are defined the same as in Formula (I) above;

Lv₁ and Lv₂ are independently a reacting group that can be reacted with a thiol, amine, carboxylic acid, selenol, phenol or hydroxyl group on a cell-binding molecule. Such reacting groups are, but are not limited to, a halide (e.g., fluoride, chloride, bromide, and iodide), methanesulfonyl (mesyl), toluenesulfonyl (tosyl), trifluoromethyl-sulfonyl (triflate), trifluoro-methylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluoro-phenoxyl, pentachloro-phenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or an intermediate molecule generated with a condensation reagent for Mitsunobu reactions. The examples of condensation reagents are: EDC (N-(3-Dimethyl-aminopropyl)-N′-ethylcarbodiimide), DCC (Dicyclohexyl-carbodiimide), N,N′-Diisopropyl-carbodiimide (DIC), N-Cyclohexyl-N′-(2-morpholino-ethyl)carbodiimide metho-p-toluenesulfonate (CMC, or CME-CDI), 1,1′-Carbonyldiimidazole (CDI), TBTU (O-(Benzotriazol-1-yl)-N,N,N′,N′-tetramethyluronium tetrafluoroborate), N,N,N′,N′-Tetramethyl-O-(1H-benzotriazol-1-yl)-uronium hexafluorophosphate (HBTU), (Benzotriazol-1-yloxy)tris-(dimethylamino)-phosphonium hexafluorophosphate (BOP), (Benzotriazol-1-yloxy)tripyrrolidinophosphonium hexafluorophosphate (PyBOP), Diethyl cyanophosphonate (DEPC), Chloro-N,N,N′,N′-tetramethylformamidiniumhexafluorophosphate, 1-[Bis(dimethylamino)methylene]-1H-1,2,3-triazolo[4,5-b]pyridinium 3-oxid hexafluorophosphate (HATU), 1-[(Dimethylamino)(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetramethylene)formamidinium hexafluorophosphate (BTFFH), N,N,N′,N′-Tetramethyl-S-(1-oxido-2-pyridyl)thiuronium hexafluorophosphate, O-(2-Oxo-1(2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate (HOTU), (1-Cyano-2-ethoxy-2-oxoethylidenaminooxy)dimethylamino-morpholino-carbenium hexafluorophosphate (COMU), O-(Benzotriazol-1-yl)-N,N,N′,N′-bis(tetramethylene)uronium hexafluorophosphate (HBPyU), N-Benzyl-N′-cyclohexyl-carbodiimide (with, or without polymer-bound), Dipyrrolidino(N-succinimidyl-oxy)carbenium hexafluoro-phosphate (HSPyU), Chlorodipyrrolidinocarbenium hexafluoro-phosphate (PyClU), 2-Chloro-1,3-dimethylimidazolidinium tetrafluoroborate(CIB), (Benzotriazol-1-yloxy)dipiperidino-carbenium hexafluorophosphate (HBPipU), O-(6-Chlorobenzotriazol-1-yl)-N,N,N′,N′-tetramethyluronium tetrafluoroborate (TCTU), Bromotris(dimethylamino)-phosphonium hexafluorophosphate (BroP), Propylphosphonic anhydride (PPACA, T3P©), 2-Morpholinoethyl isocyanide (MEI), N,N,N′,N′-Tetramethyl-O—(N-succinimidyl)uronium hexafluorophosphate (HSTU), 2-Bromo-1-ethyl-pyridinium tetrafluoro-borate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholiniumchloride (MMTM, DMTMM), N,N,N′,N′-Tetramethyl-O—(N-succinimidyl)uronium tetrafluoroborate (TSTU), O-(3,4-Dihydro-4-oxo-1,2,3-benzotriazin-3-yl)-N,N,N′,N′-tetramethyluronium tetrafluoro-borate (TDBTU), 1,1′-(Azodicarbonyl)-dipiperidine (ADD), Di-(4-chlorobenzyl)-azodicarboxylate (DCAD), Di-tert-butyl azodicarboxylate (DBAD), Diisopropyl azodicarboxylate (DIAD), Diethyl azodicarboxylate (DEAD). In addition, Lv₁ and Lv₂ can be an anhydride, formed by acid themselves or formed with other C₁˜C₈ acid anhydrides;

Preferably Lv₁ and Lv₂ are independently selected from, a halide (e.g., fluoride, chloride, bromide, and iodide), methanesulfonyl (mesyl), toluenesulfonyl (tosyl), trifluoromethyl-sulfonyl (triflate), trifluoromethylsulfonate, nitrophenoxyl, N-succinimidyloxyl (NHS), phenoxyl; dinitrophenoxyl; pentafluorophenoxyl, tetrafluorophenoxyl, trifluorophenoxyl, difluorophenoxyl, monofluoro-phenoxyl, pentachlorophenoxyl, 1H-imidazole-1-yl, chlorophenoxyl, dichlorophenoxyl, trichlorophenoxyl, tetrachlorophenoxyl, N-(benzotriazol-yl)oxyl, 2-ethyl-5-phenylisoxazolium-3′-sulfonyl, phenyloxadiazole-sulfonyl (-sulfone-ODA), 2-ethyl-5-phenylisoxazolium-yl, phenyloxadiazol-yl (ODA), oxadiazol-yl, unsaturated carbon (a double or a triple bond between carbon-carbon, carbon-nitrogen, carbon-sulfur, carbon-phosphorus, sulfur-nitrogen, phosphorus-nitrogen, oxygen-nitrogen, or carbon-oxygen), or one of the following structure:

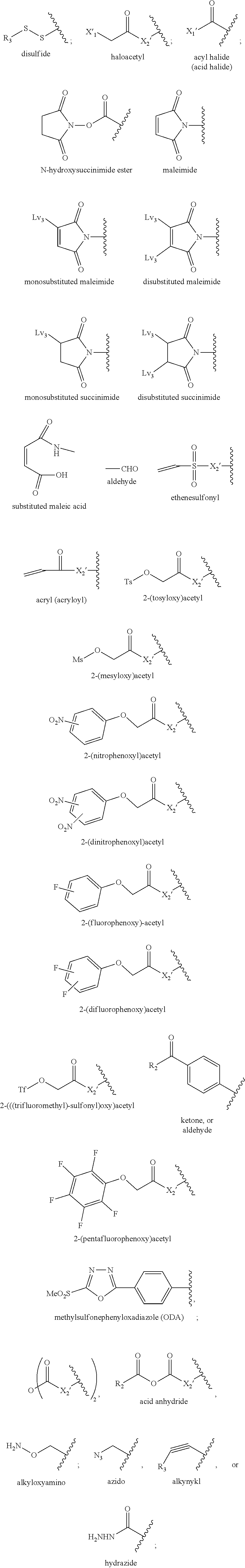

wherein X₁′ is F, Cl, Br, I or Lv₃; X₂′ is O, NH, N(R₁), or CH₂; R₃ is independently H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydrides formed its self, or formed with the other anhydride, e.g. acetyl anhydride, formyl anhydride; or an intermediate molecule generated with a condensation reagent for peptide coupling reactions or for Mitsunobu reactions.

Examples of Formula (IV), (V) and (VI) are shown below:

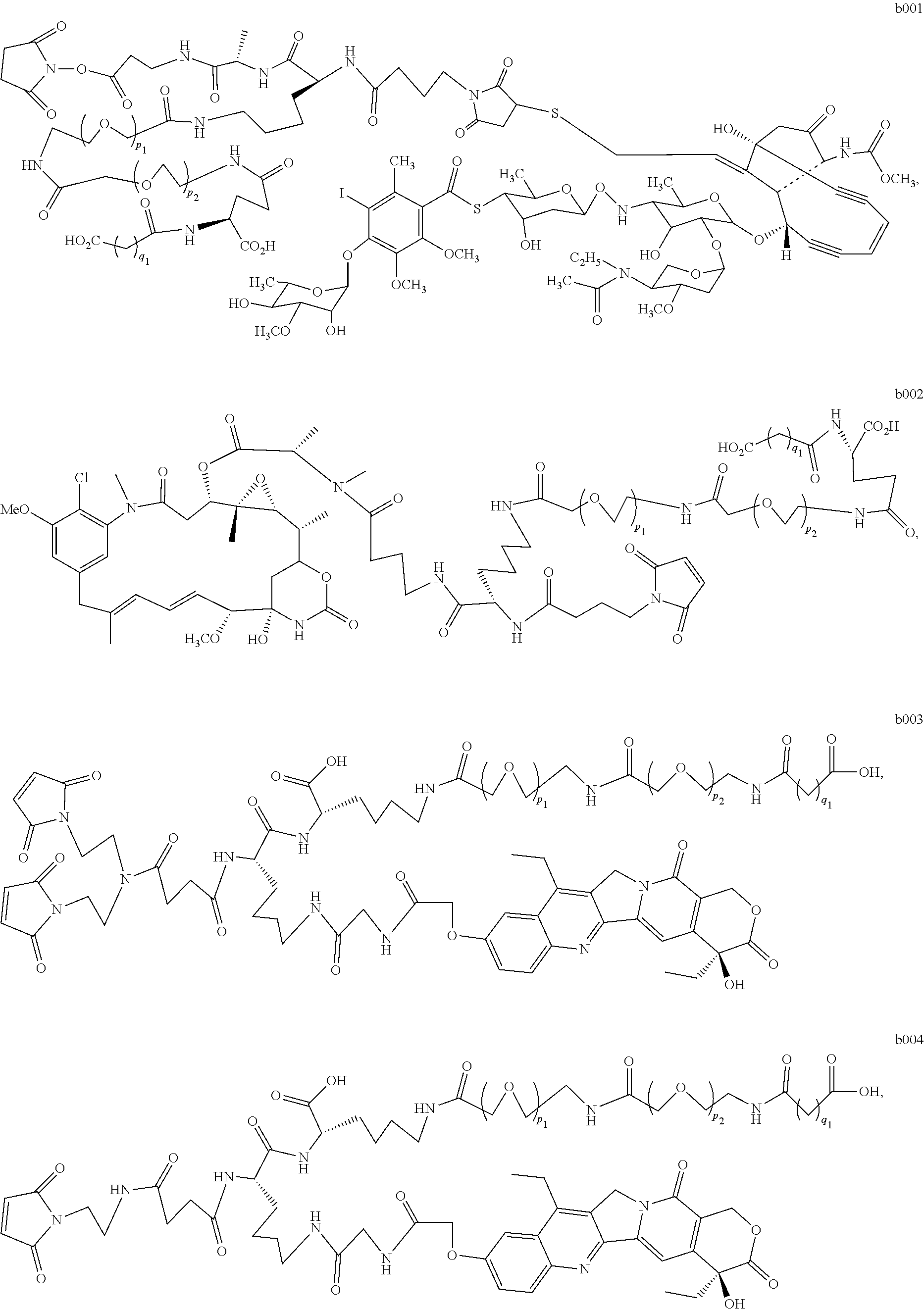

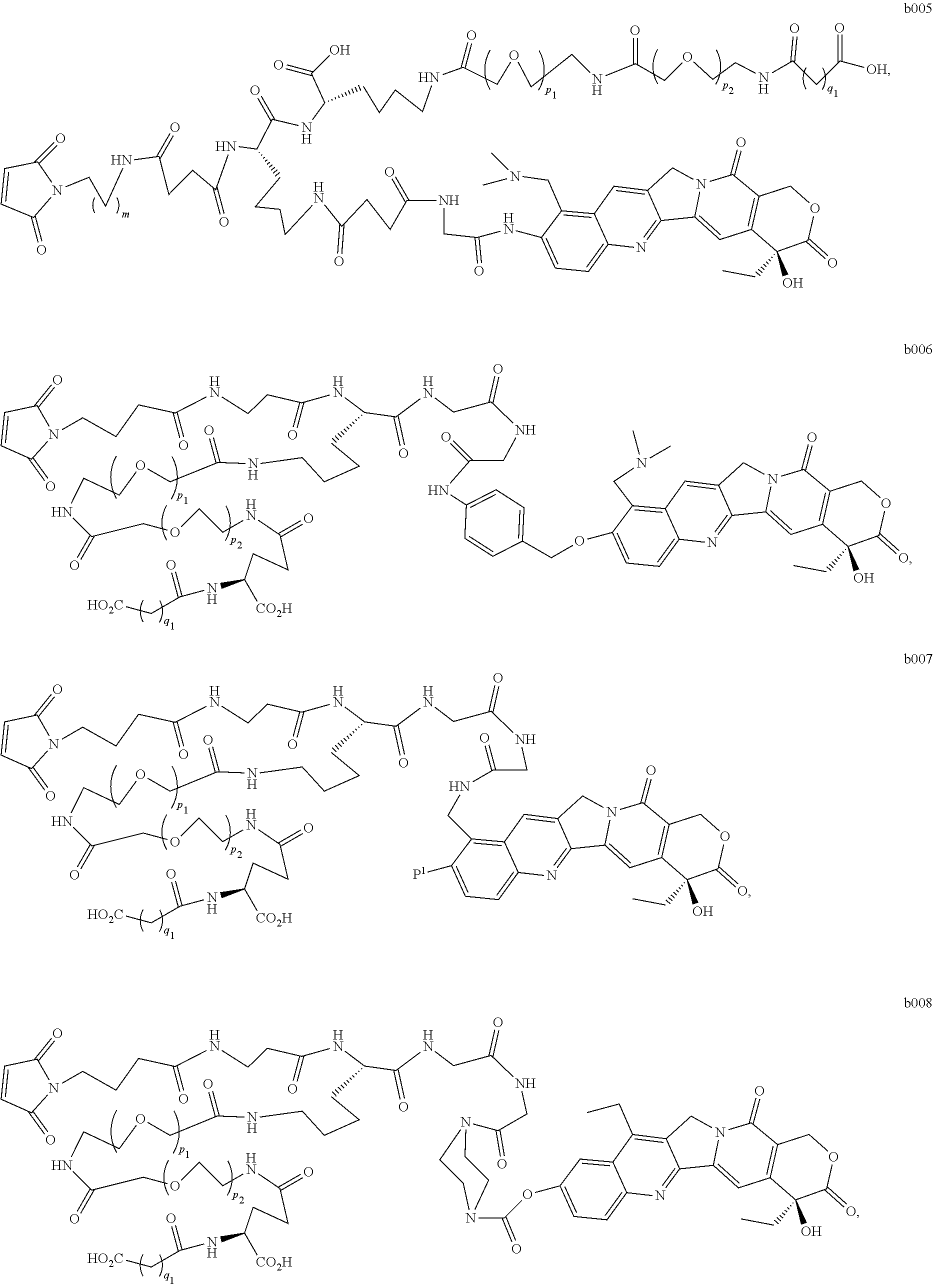

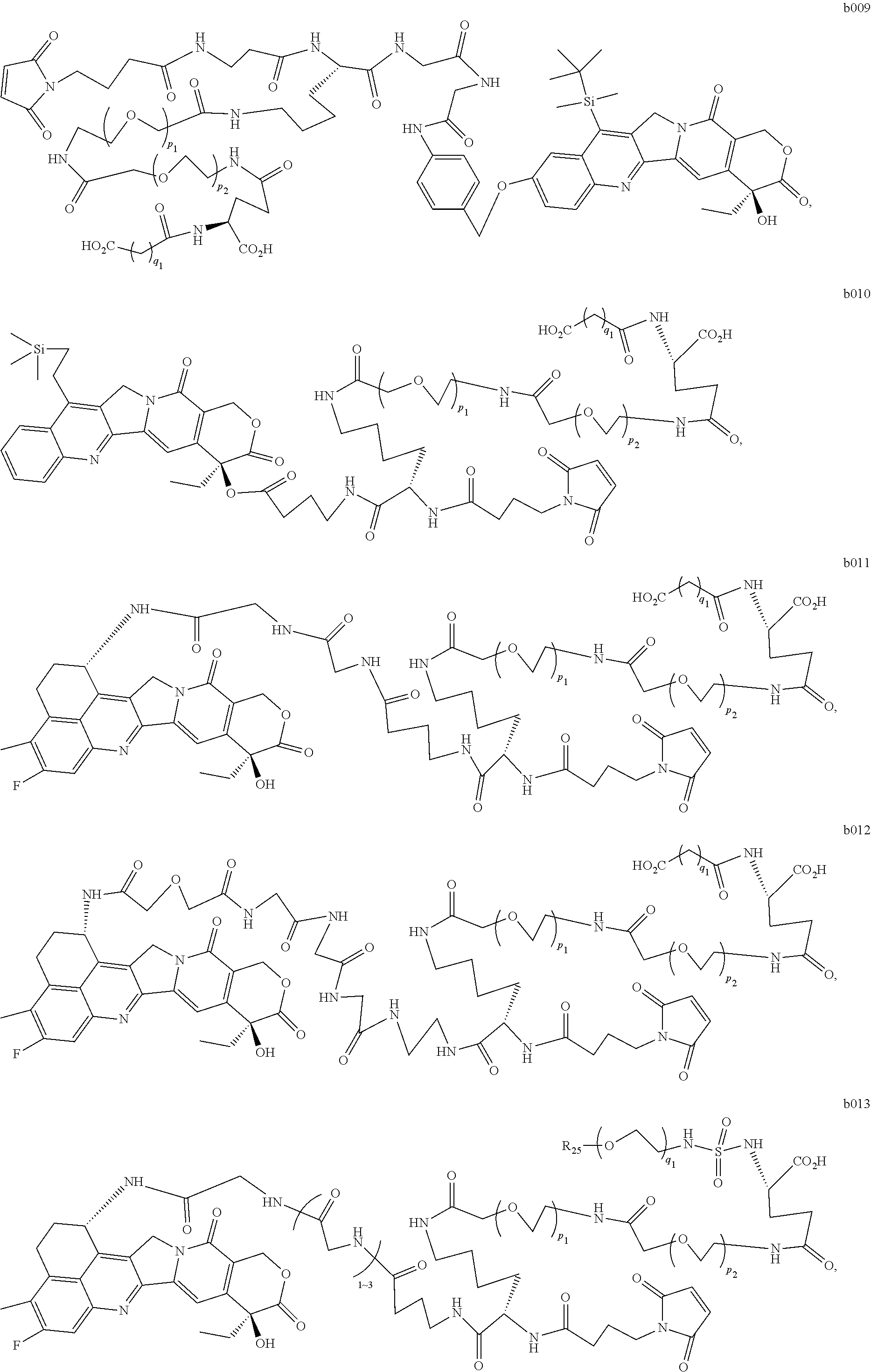

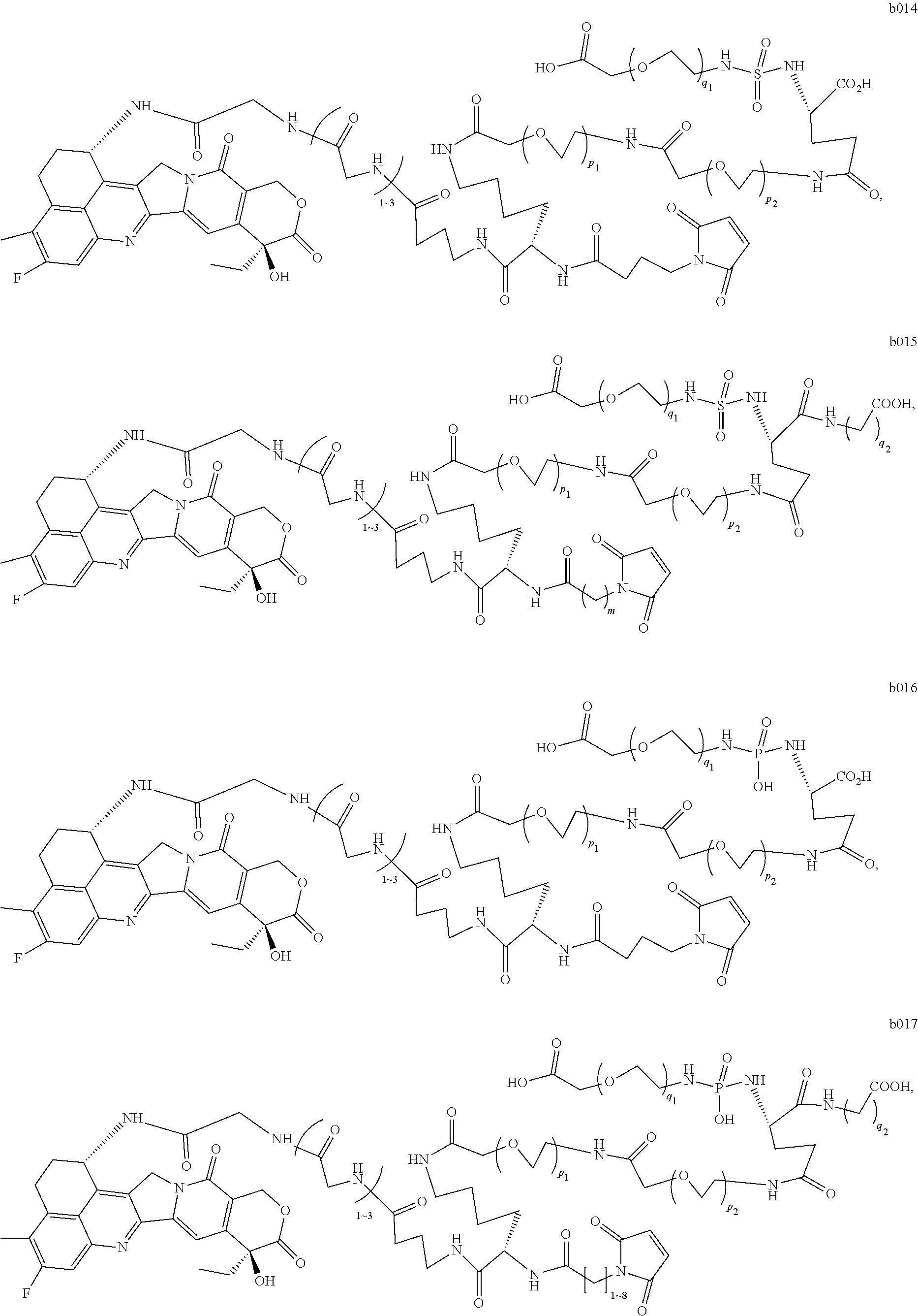

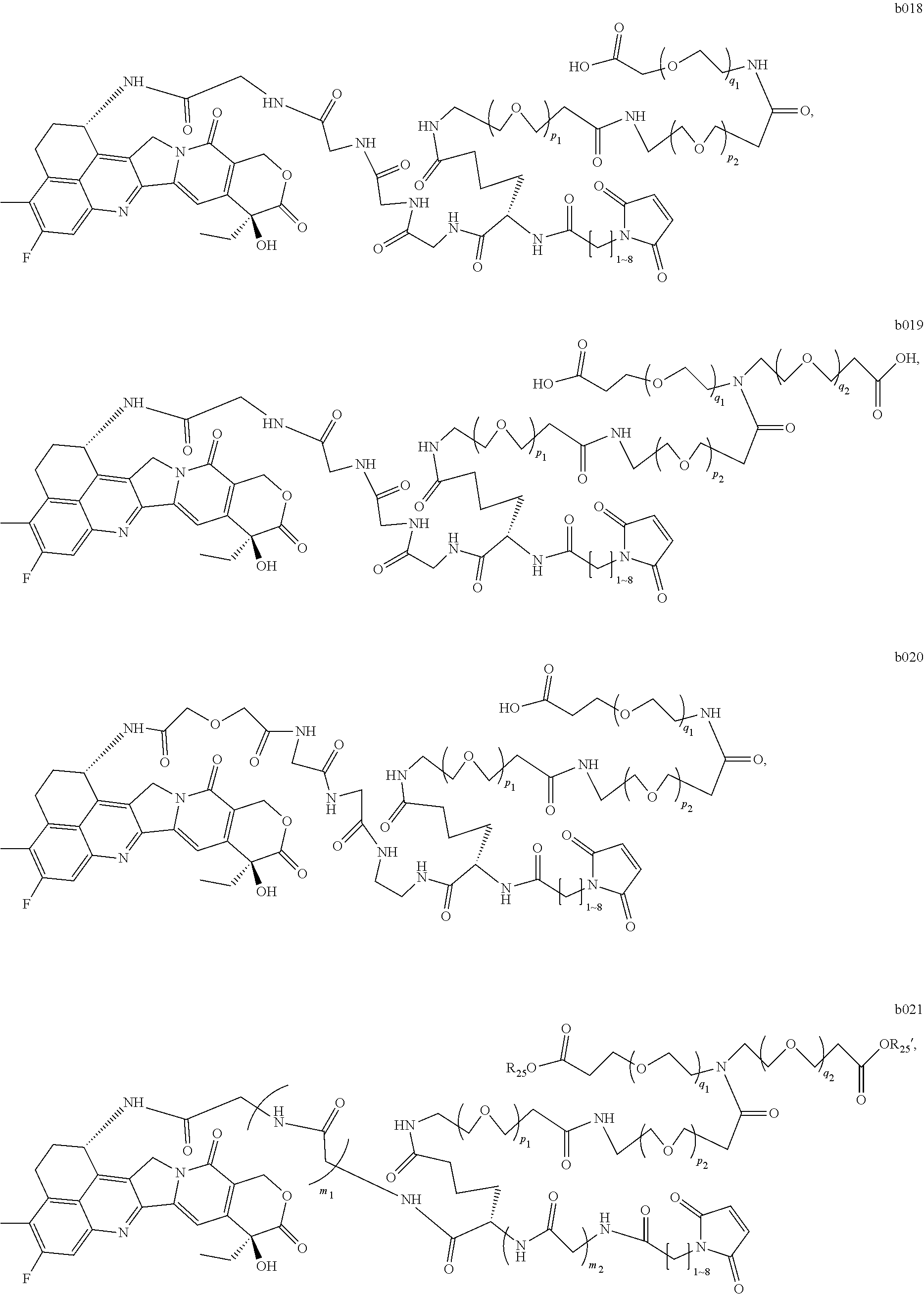

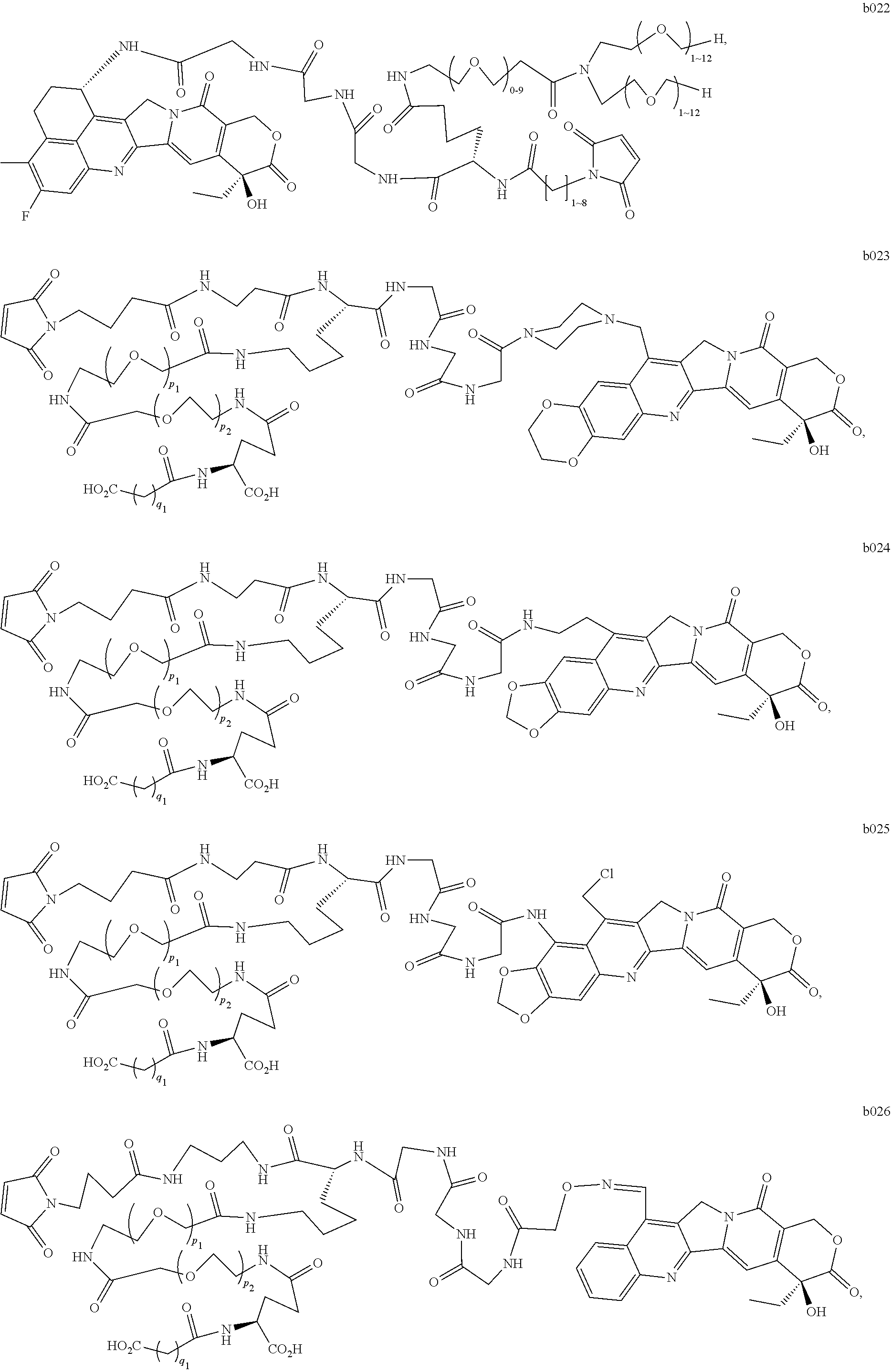

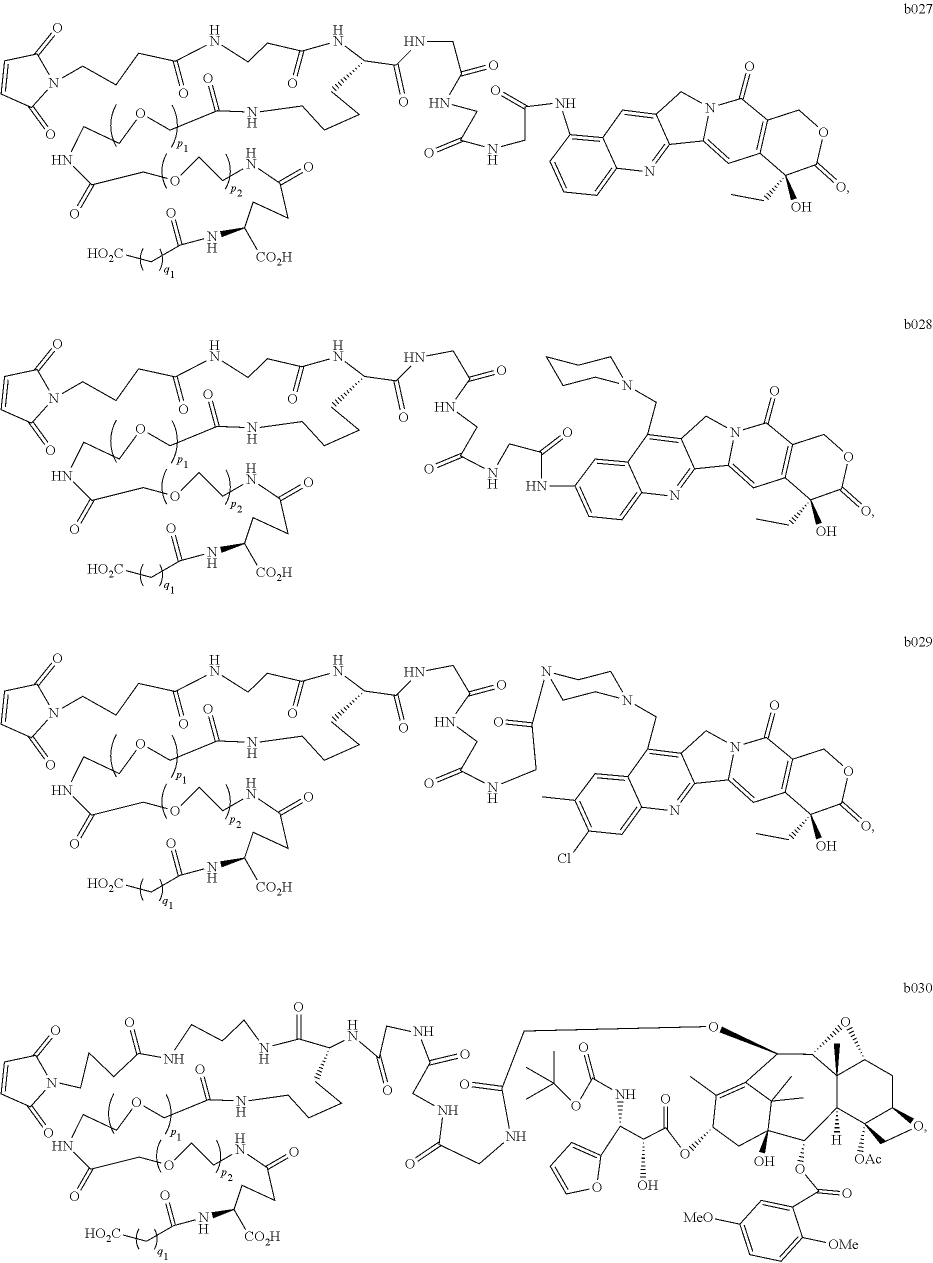

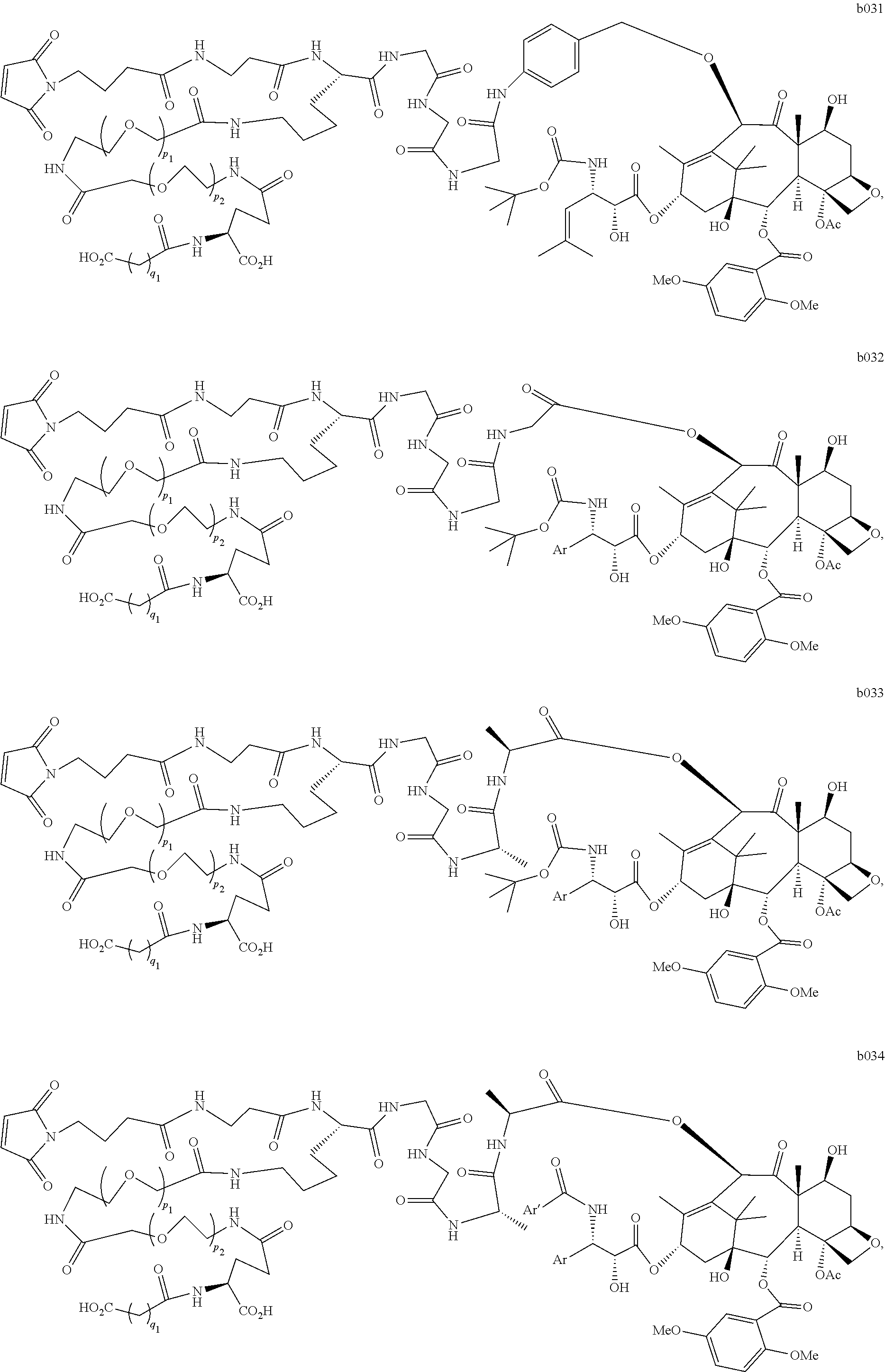

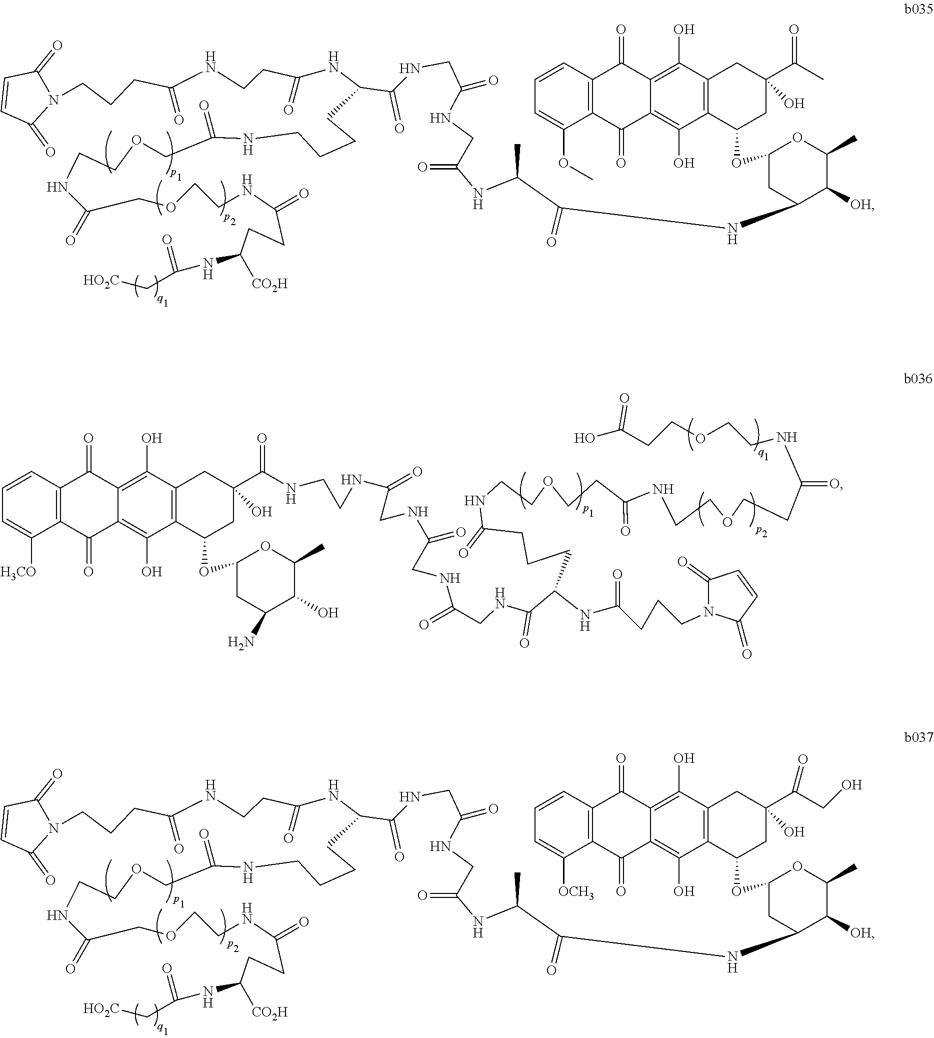

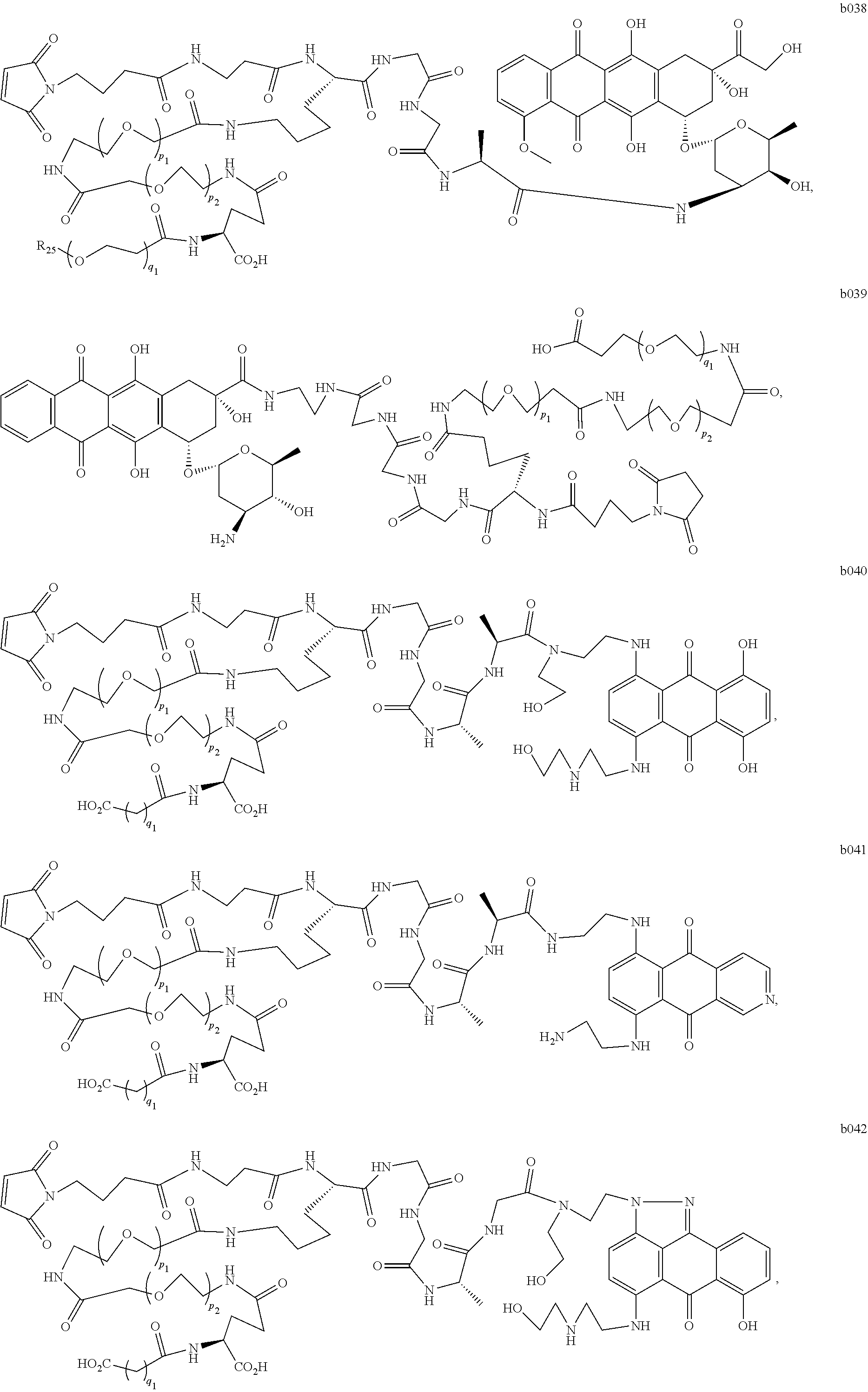

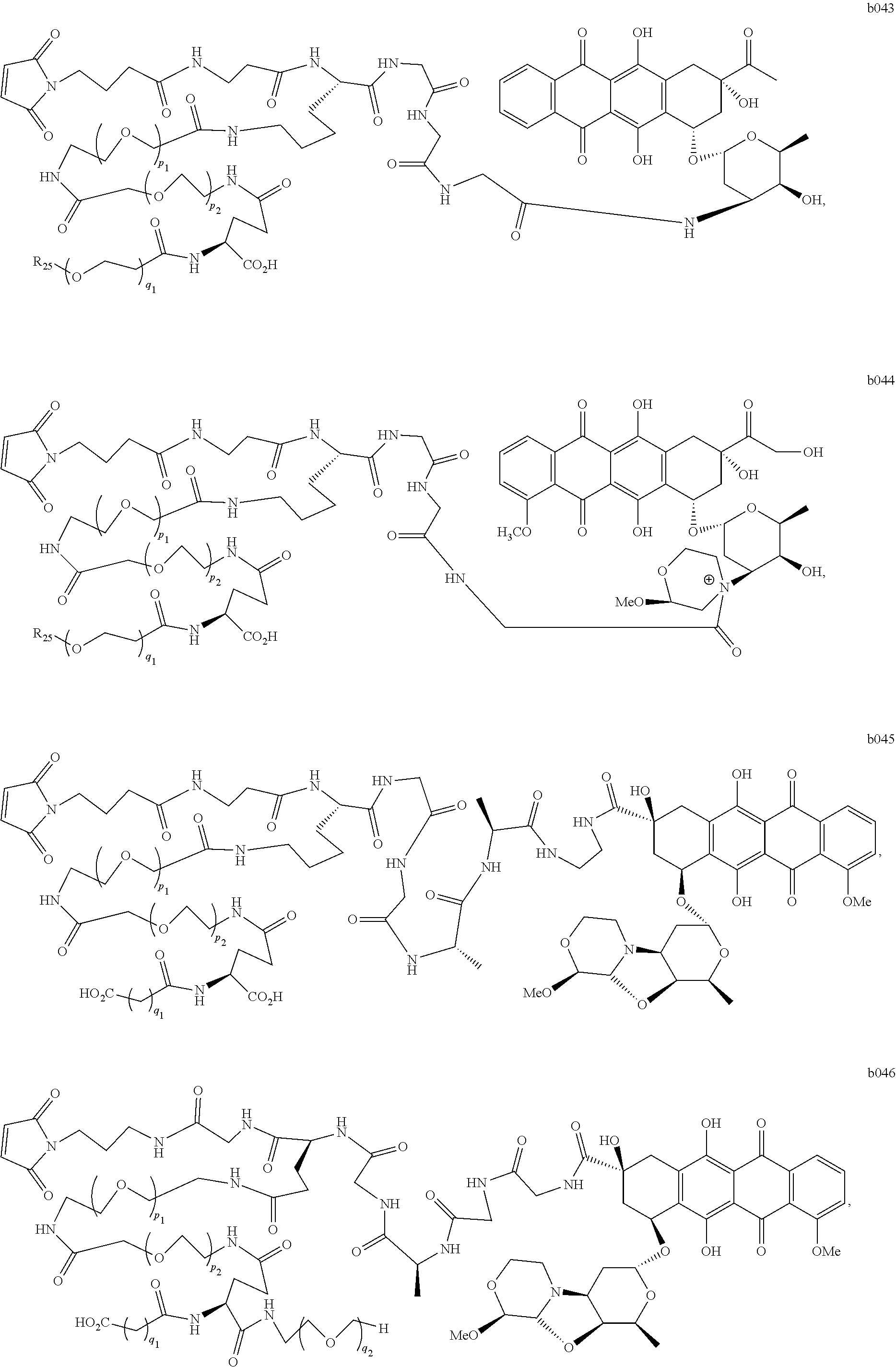

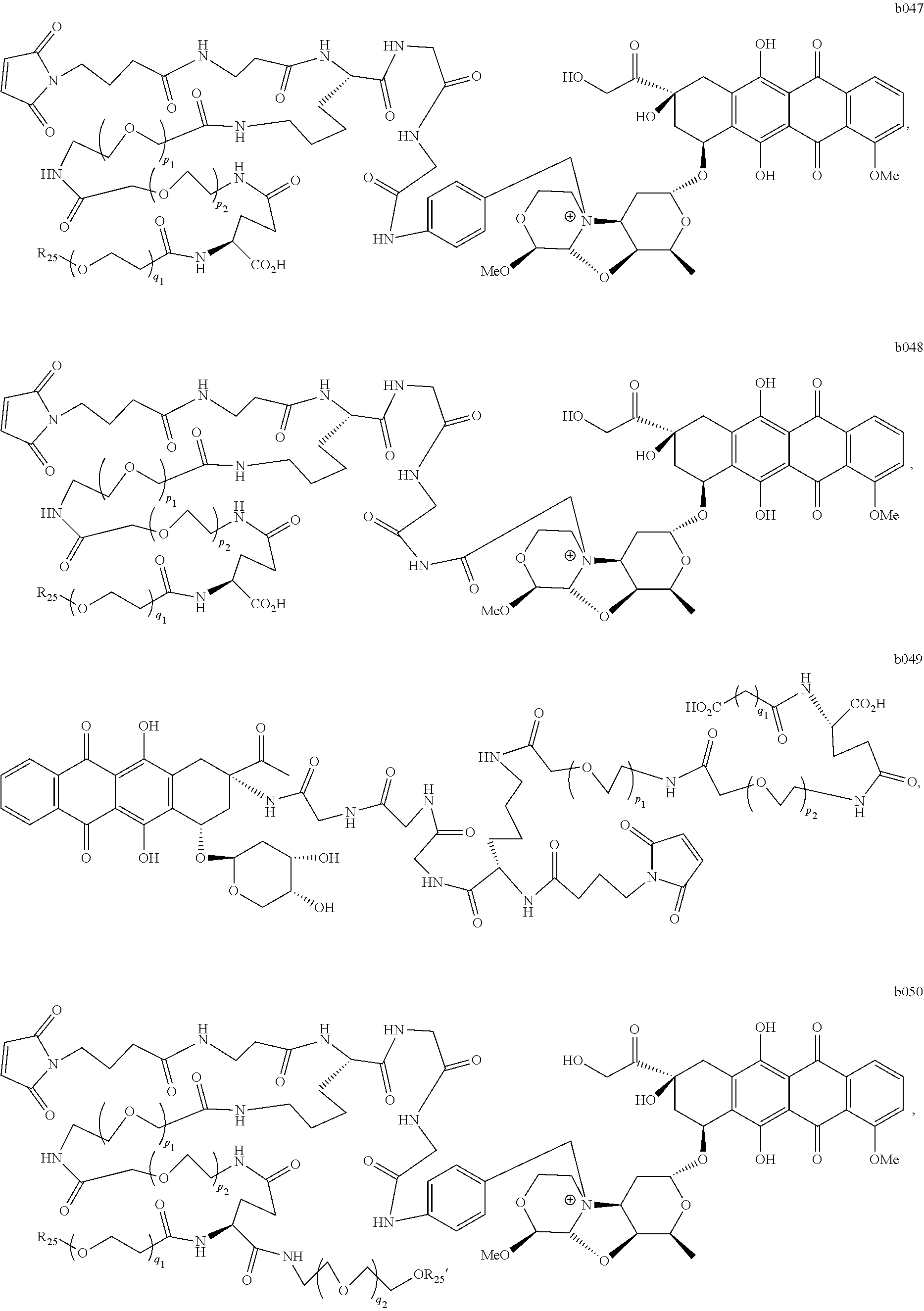

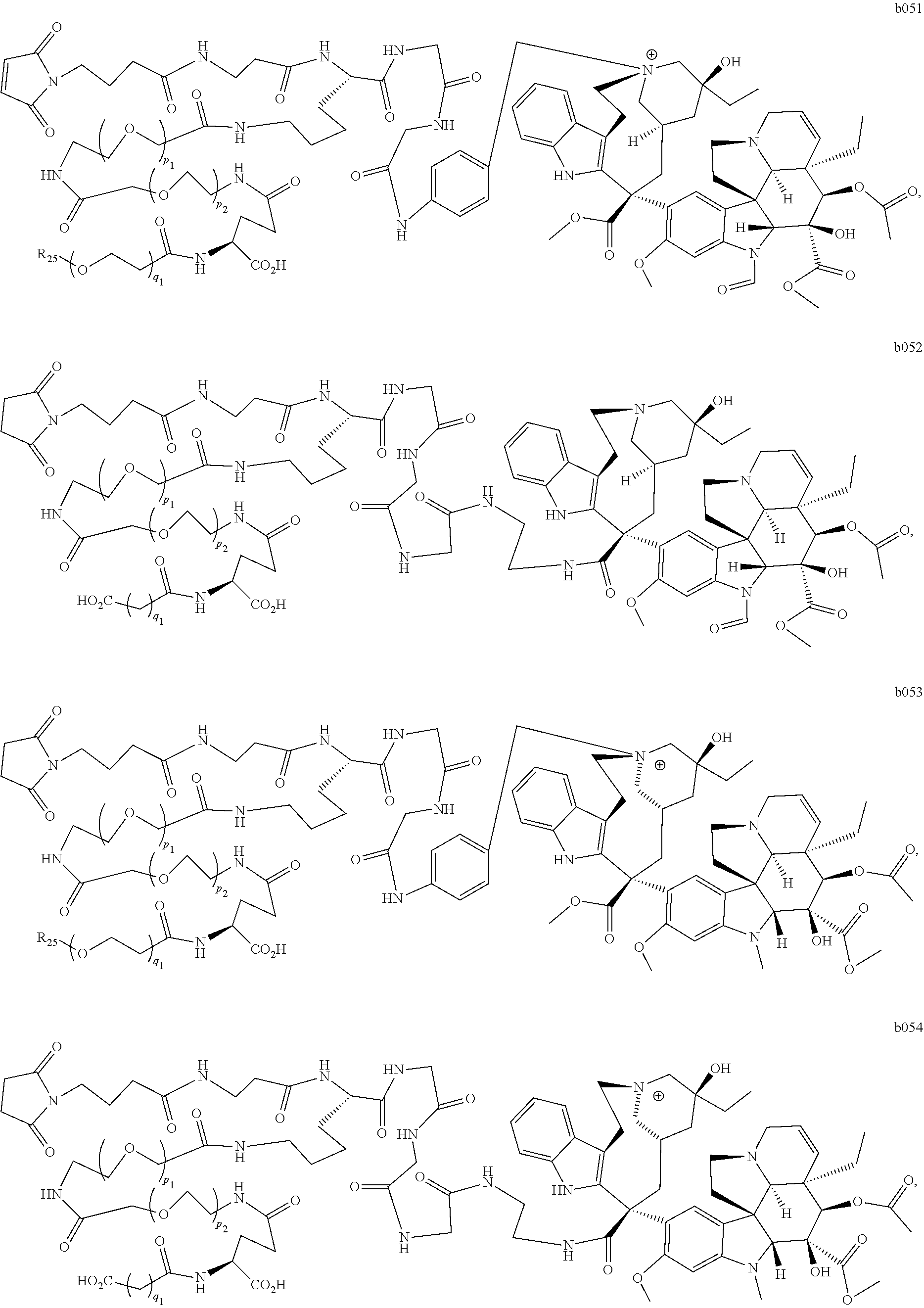

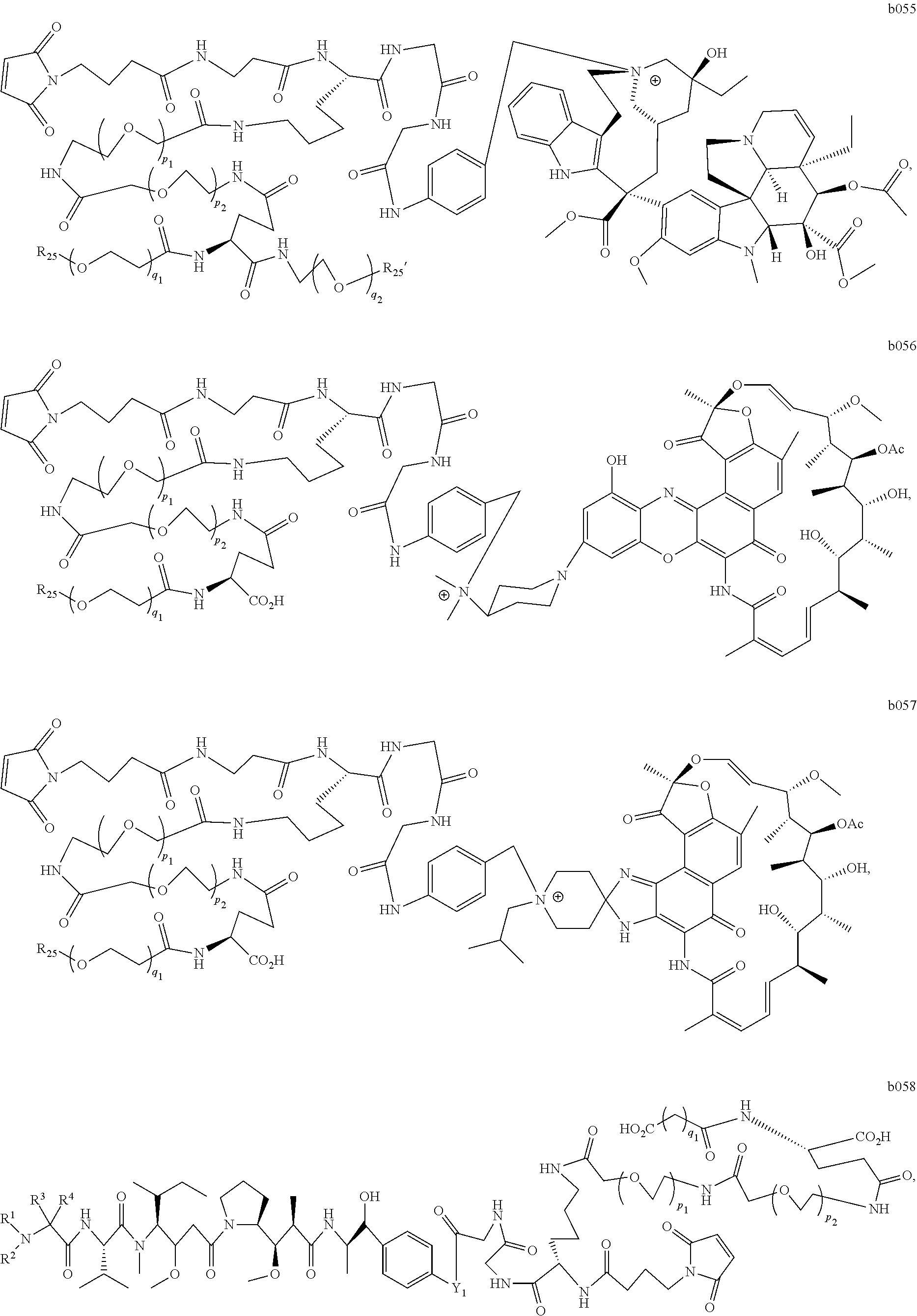

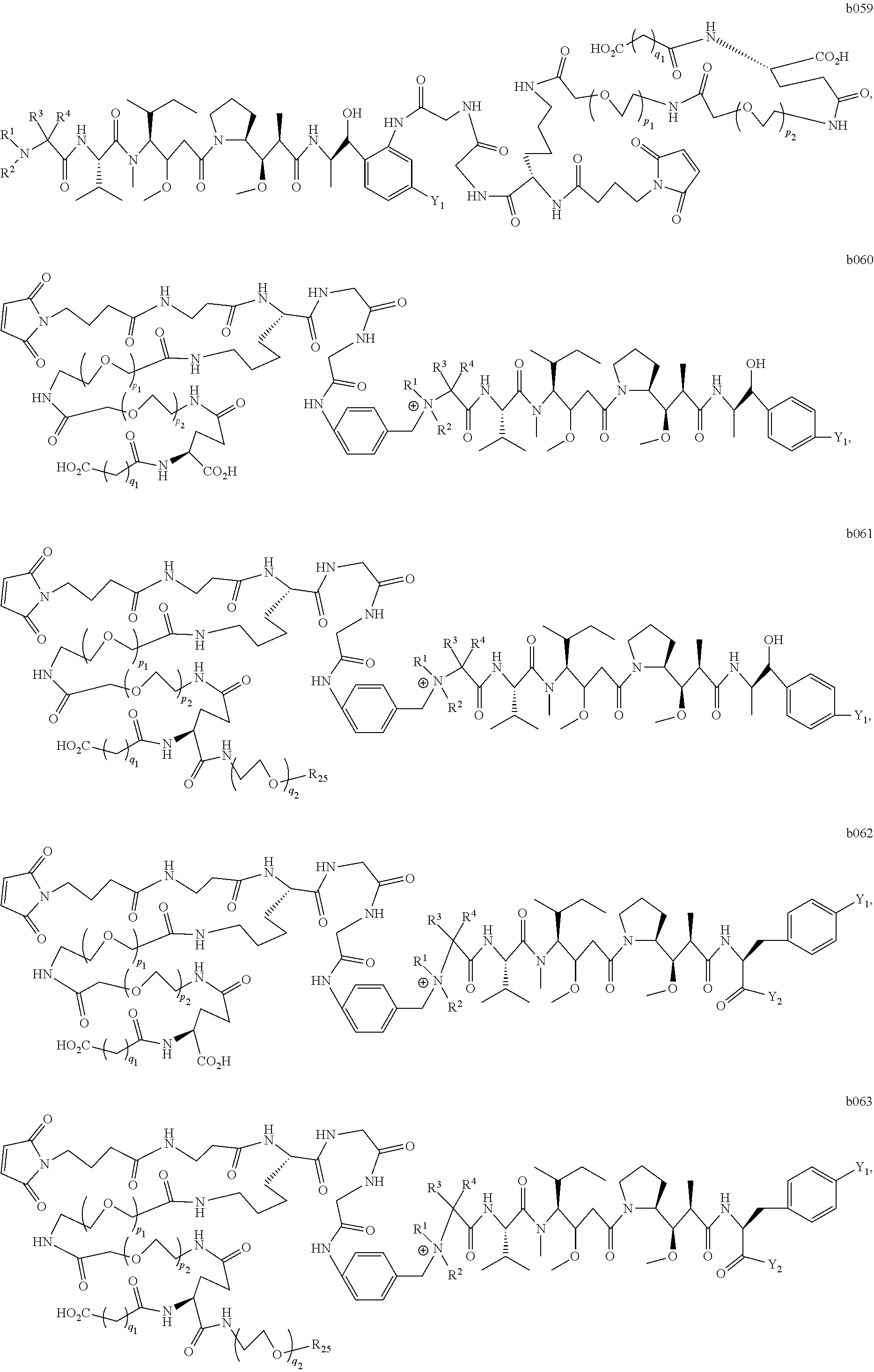

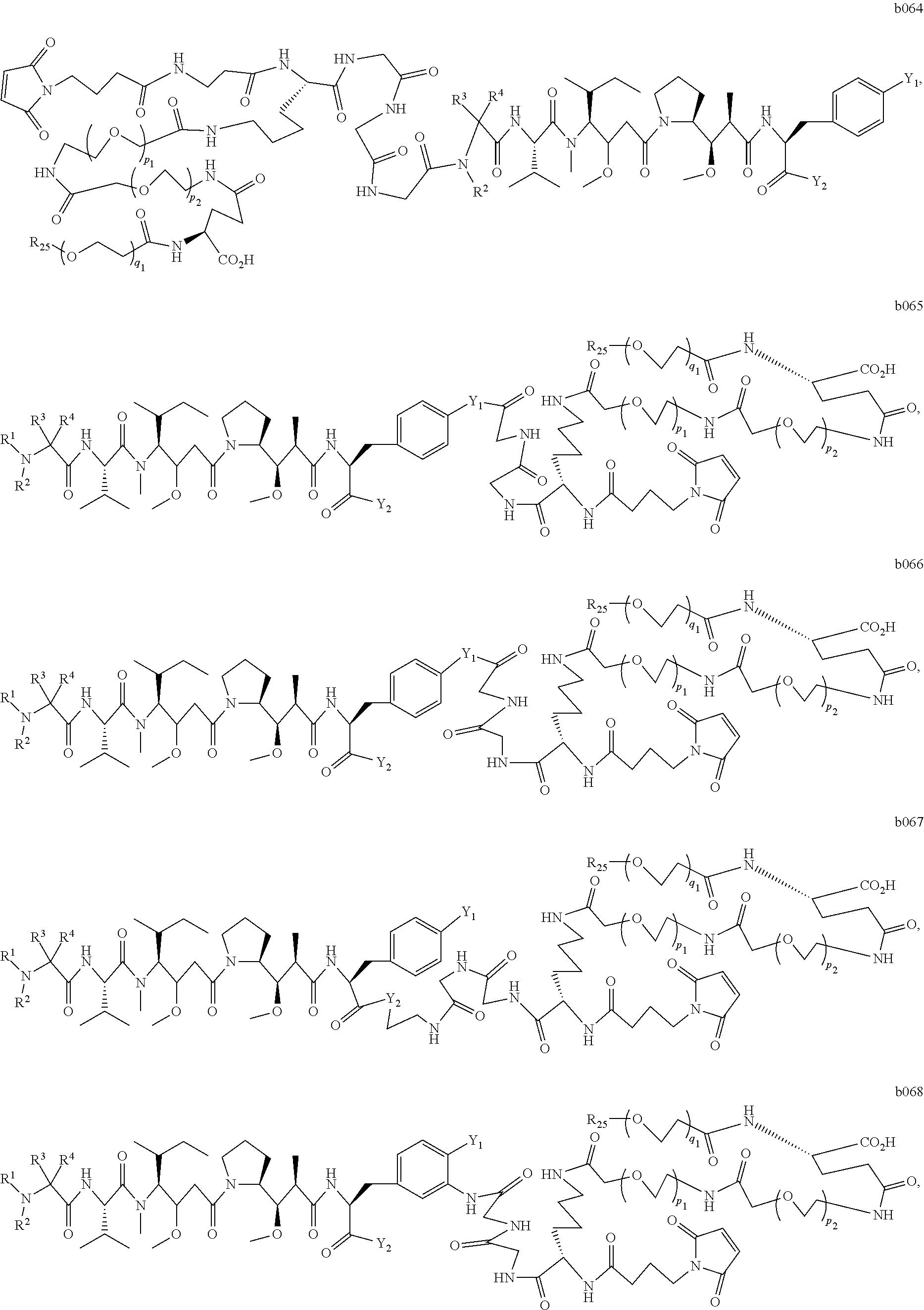

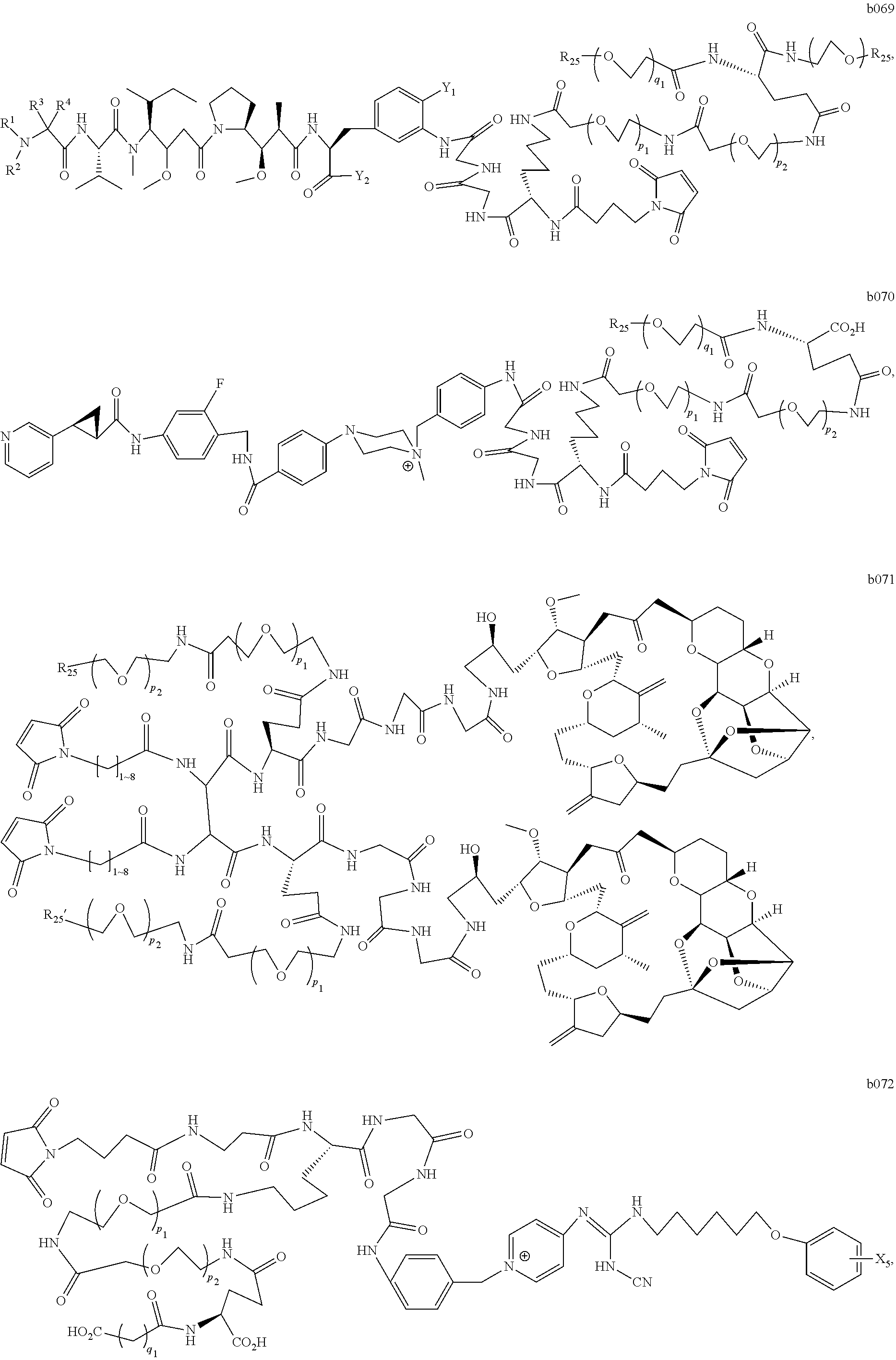

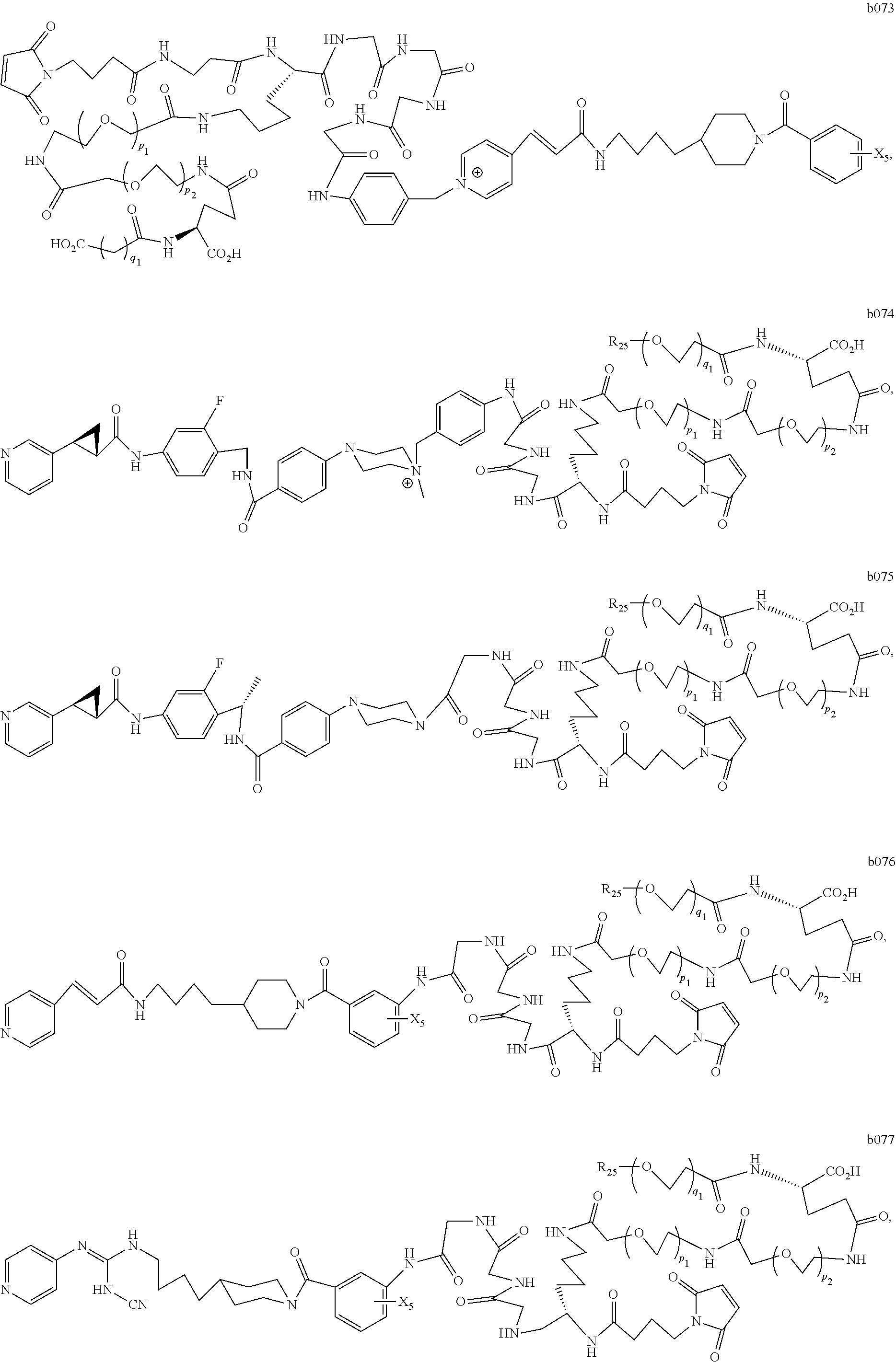

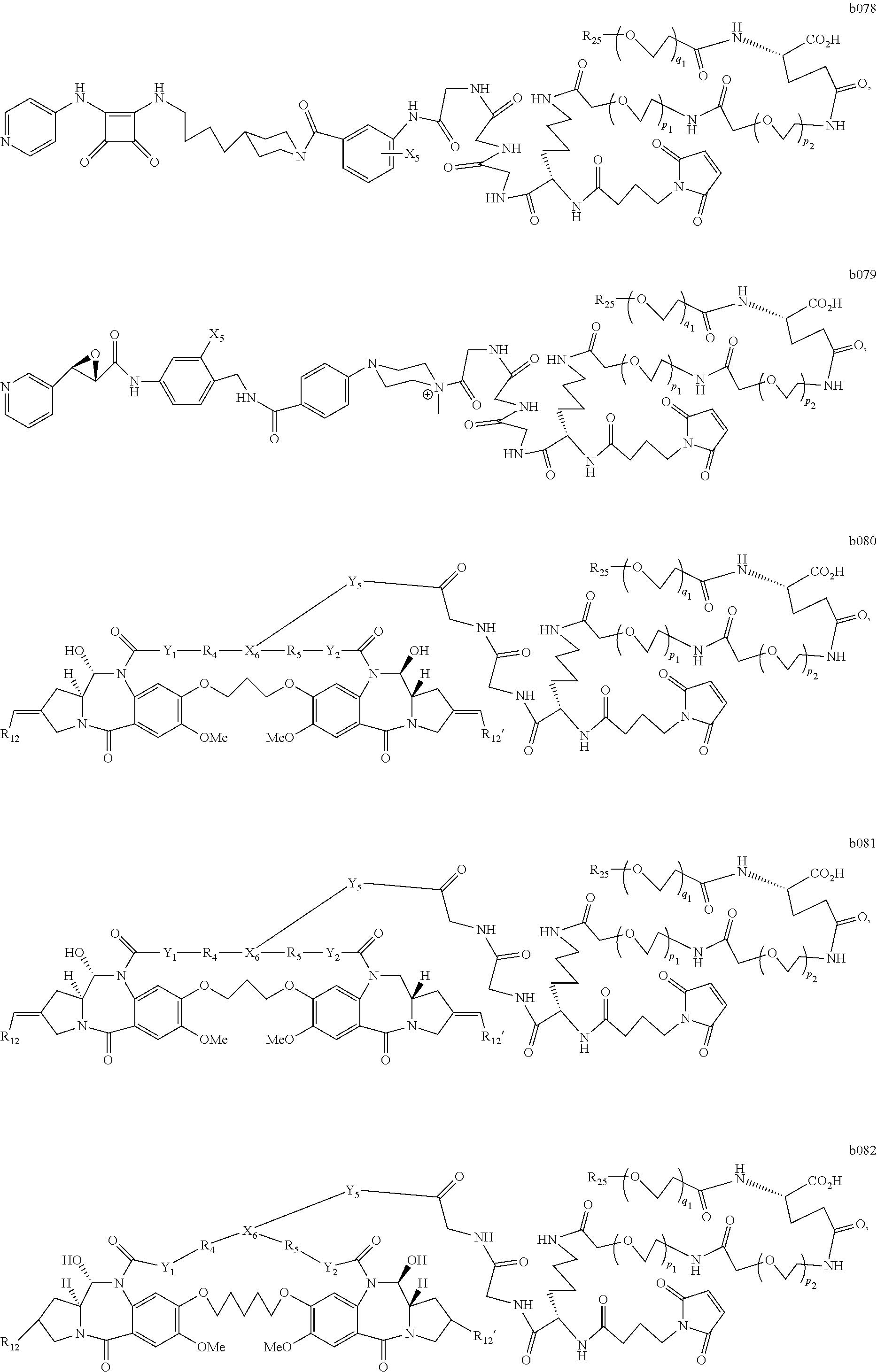

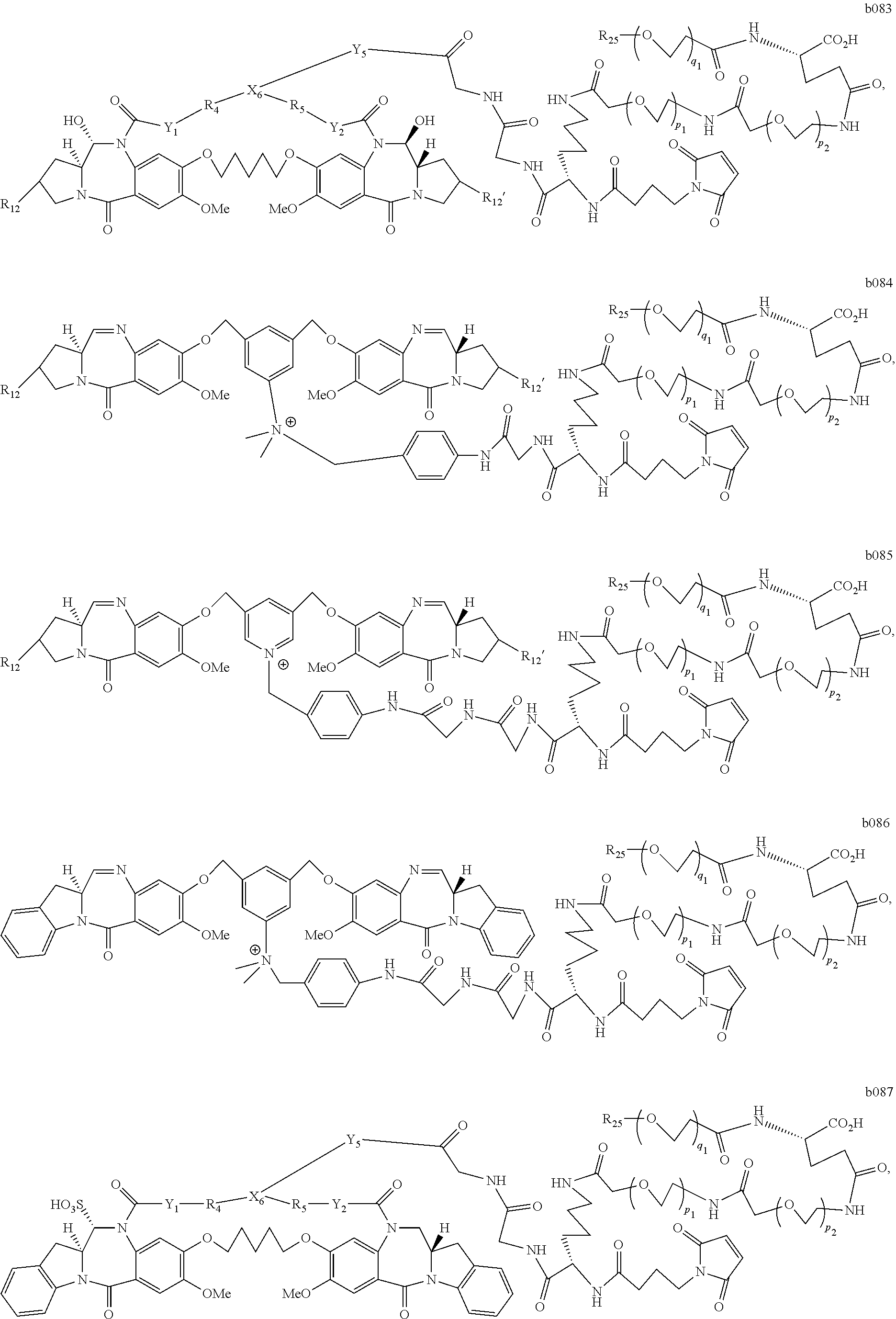

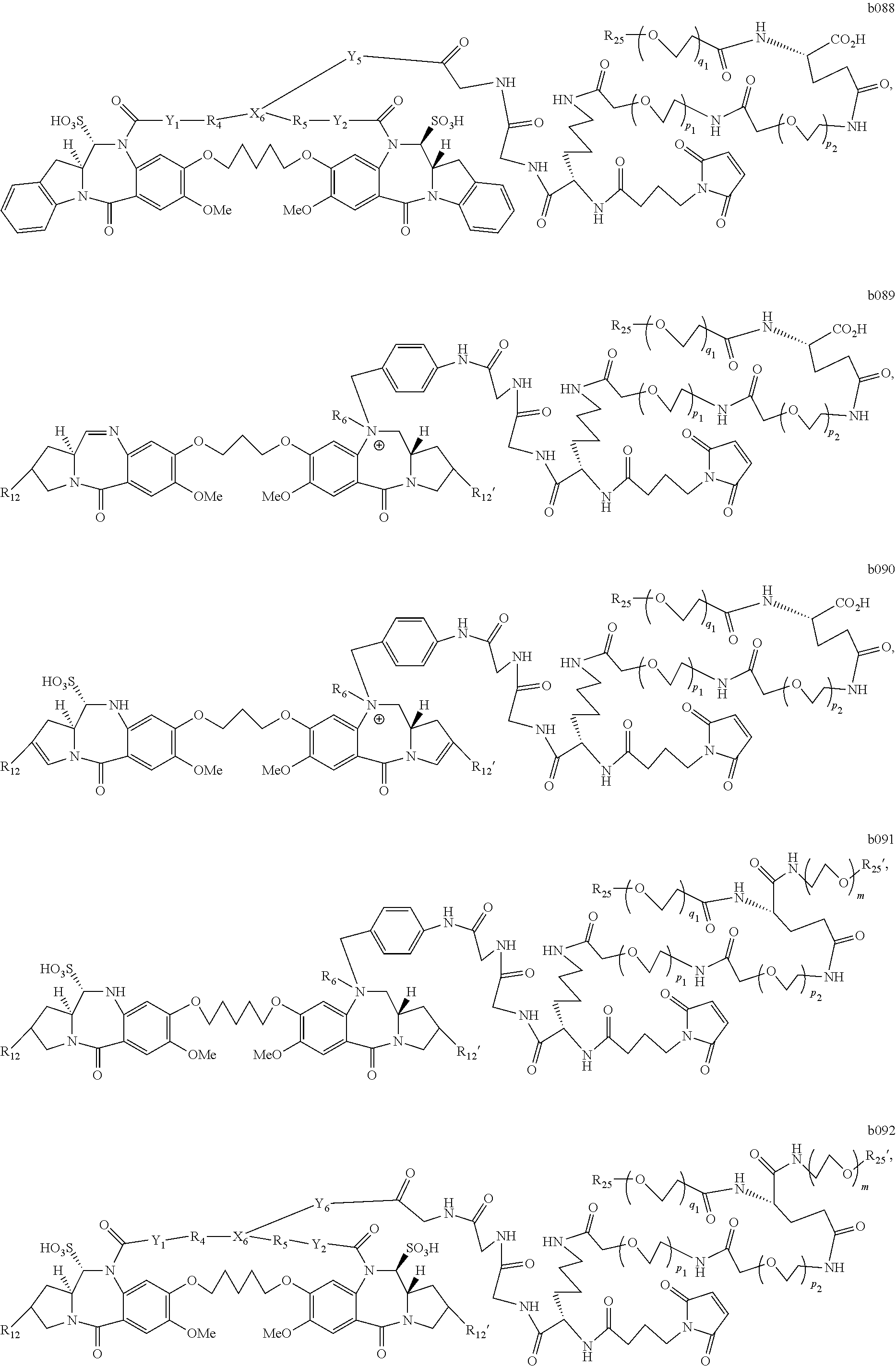

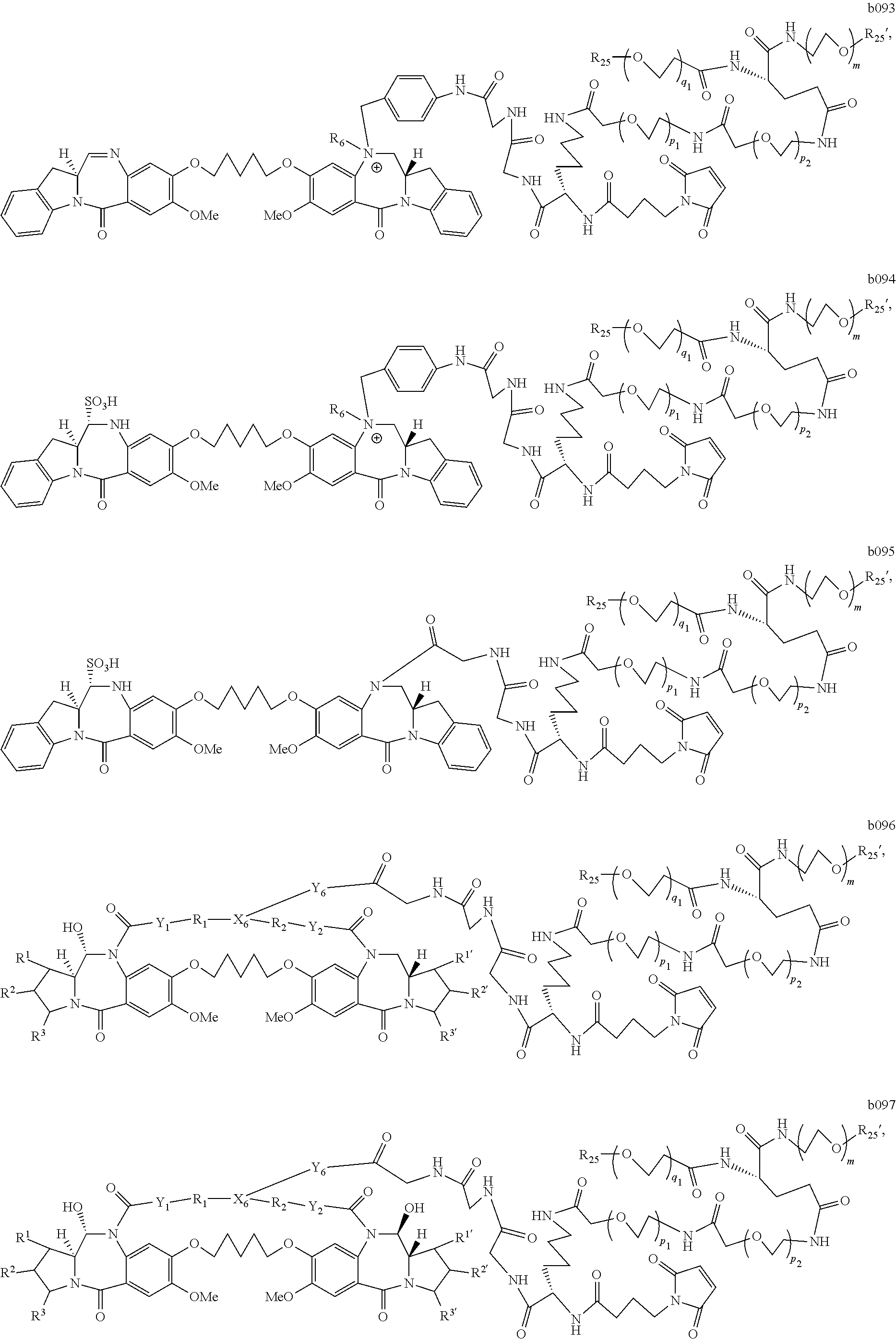

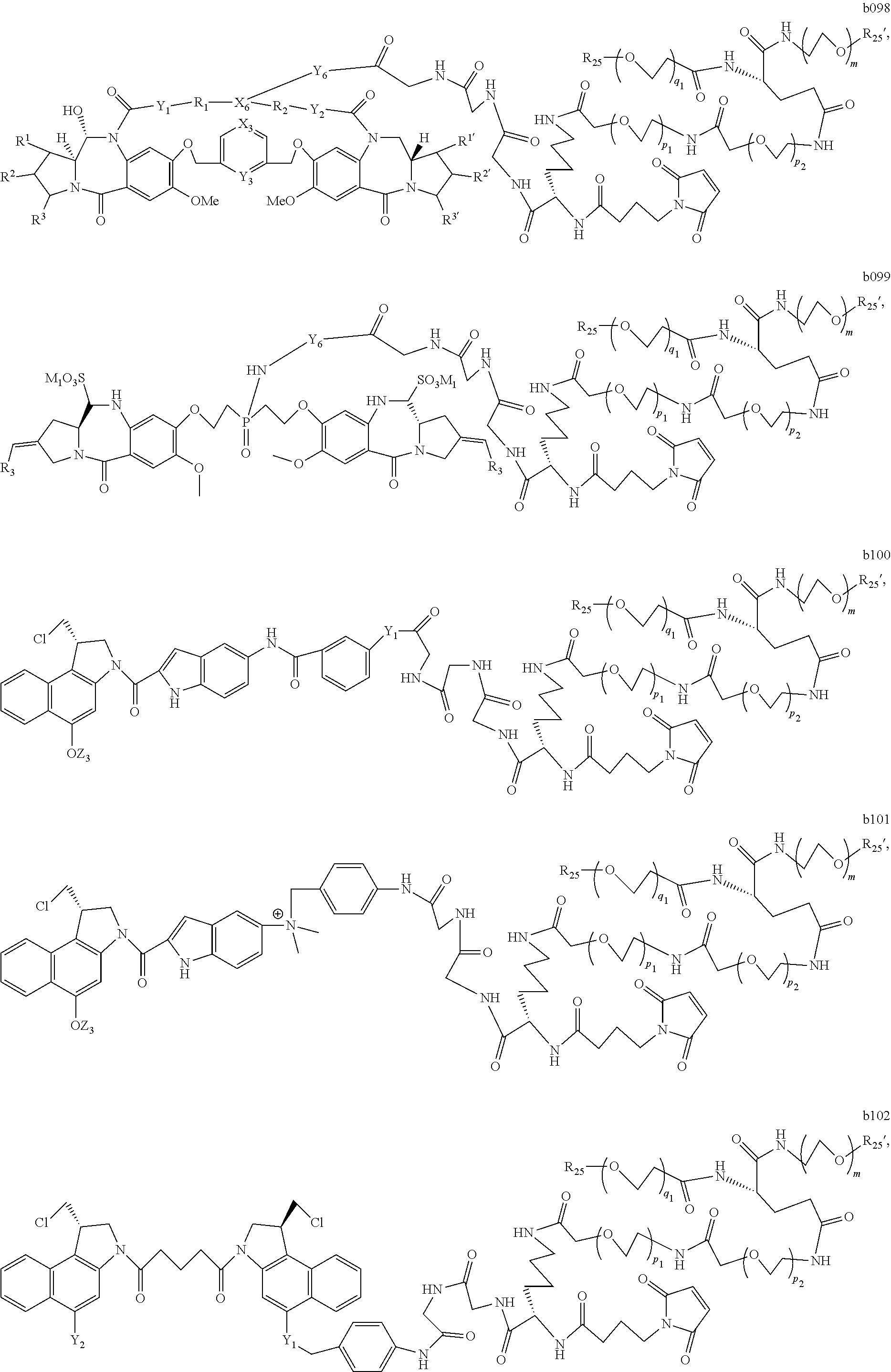

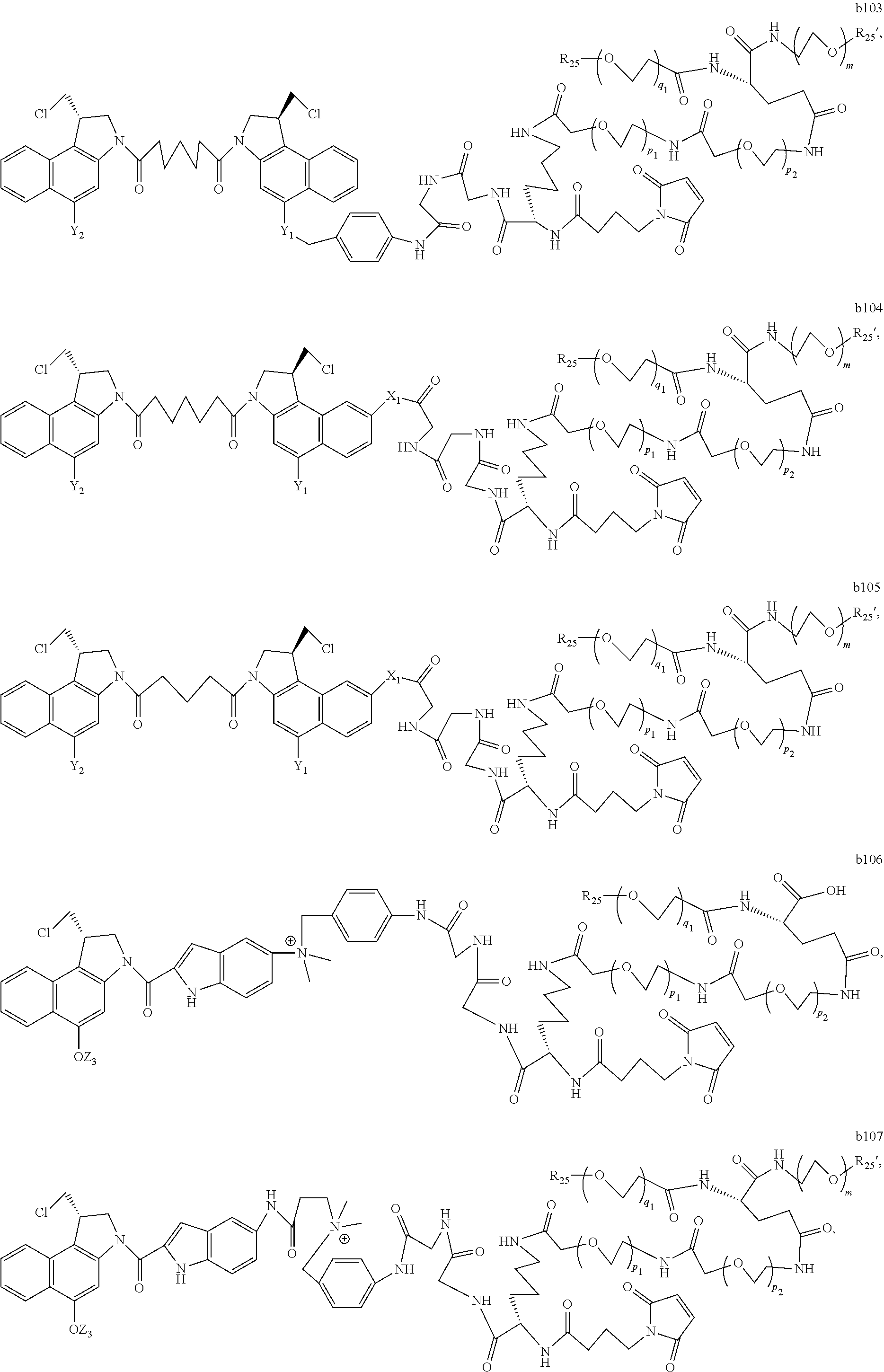

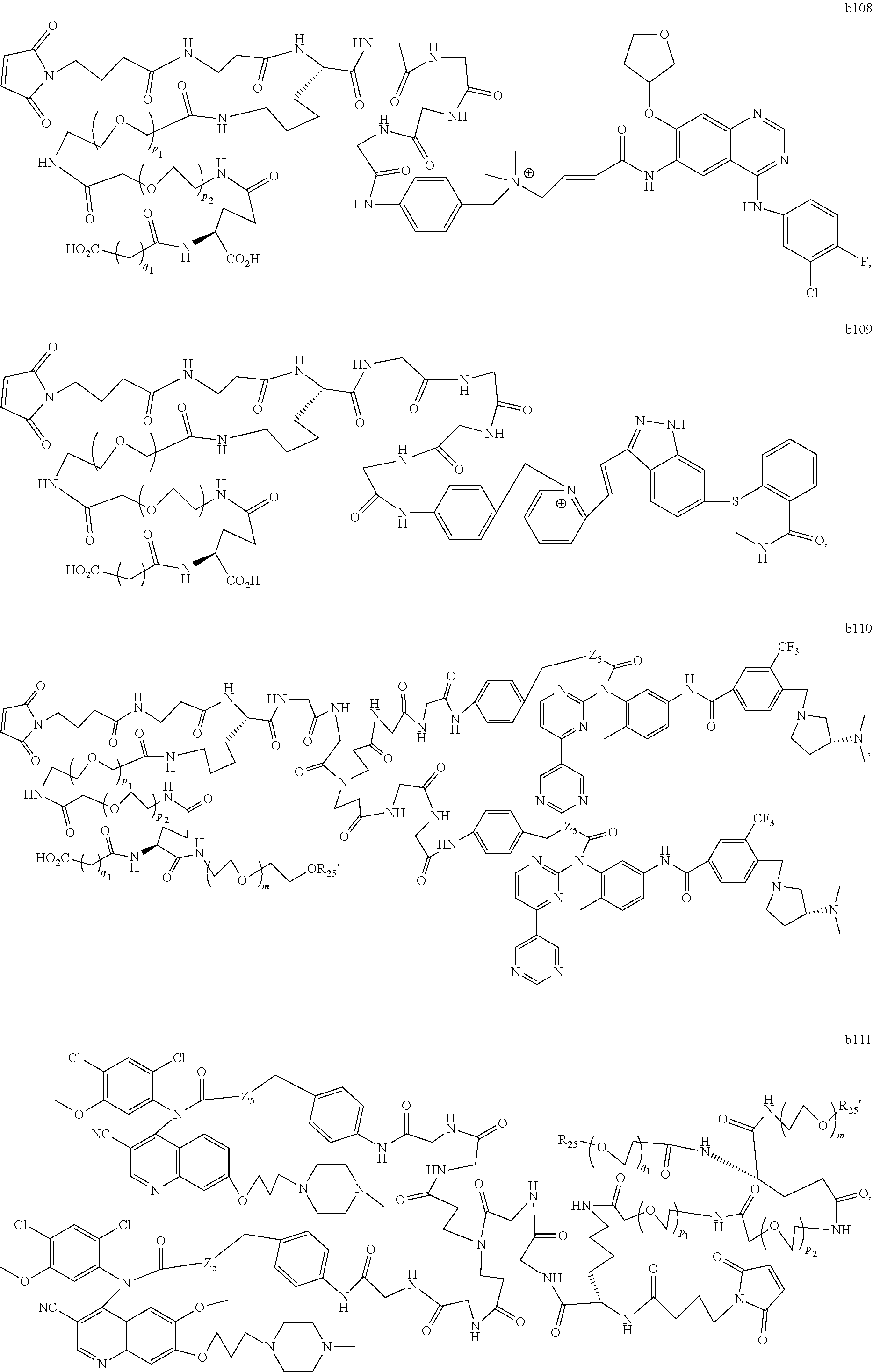

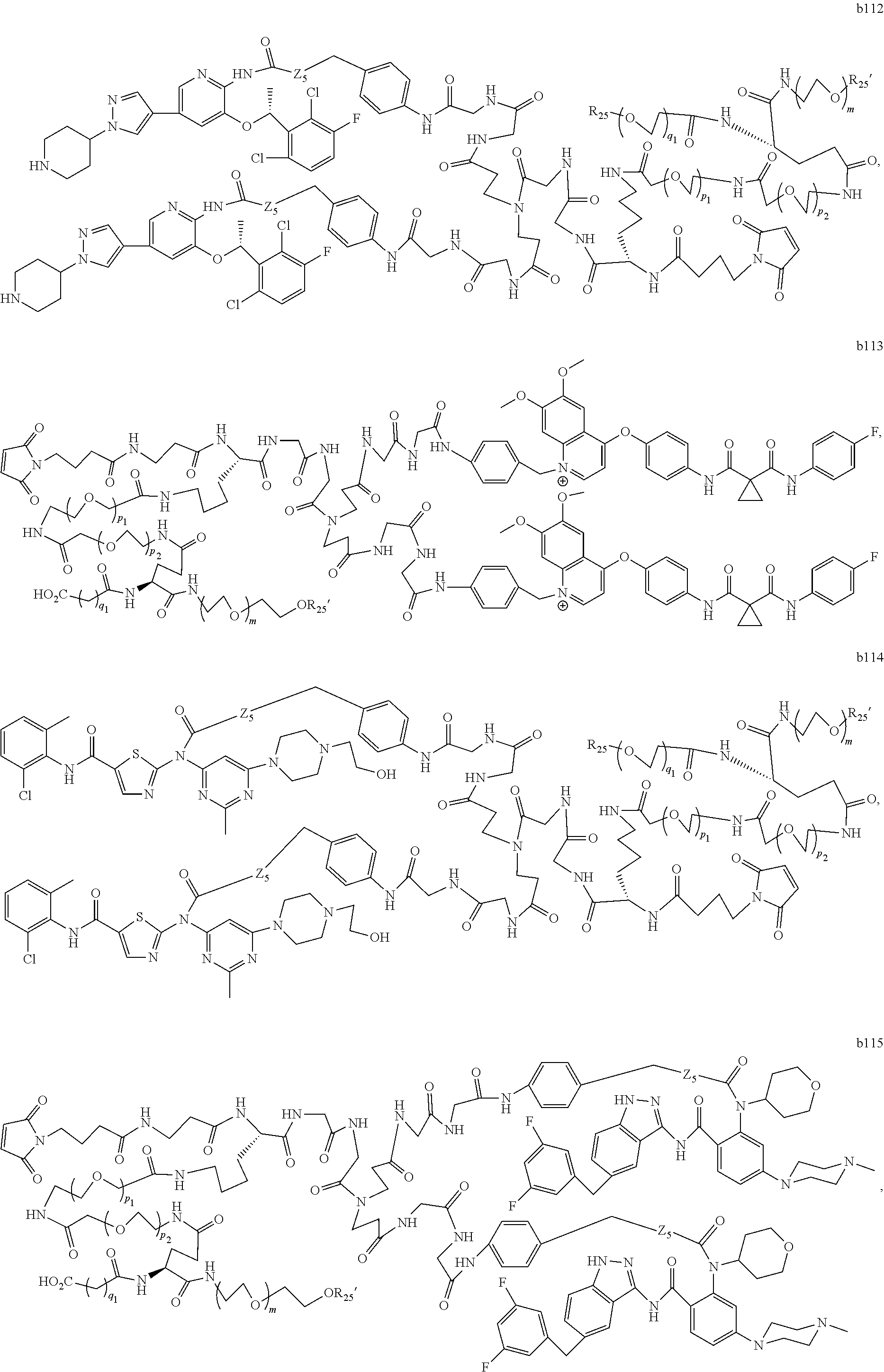

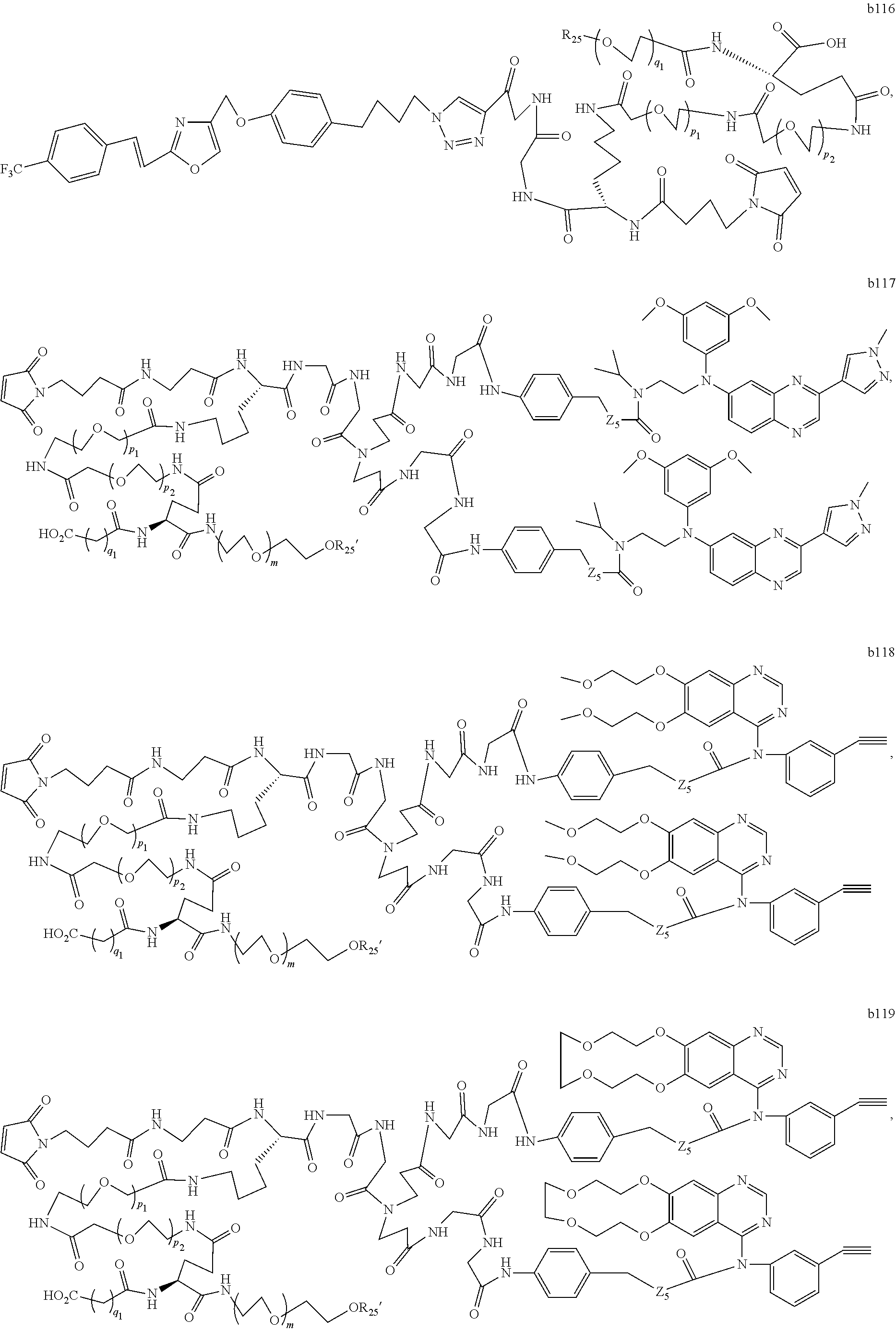

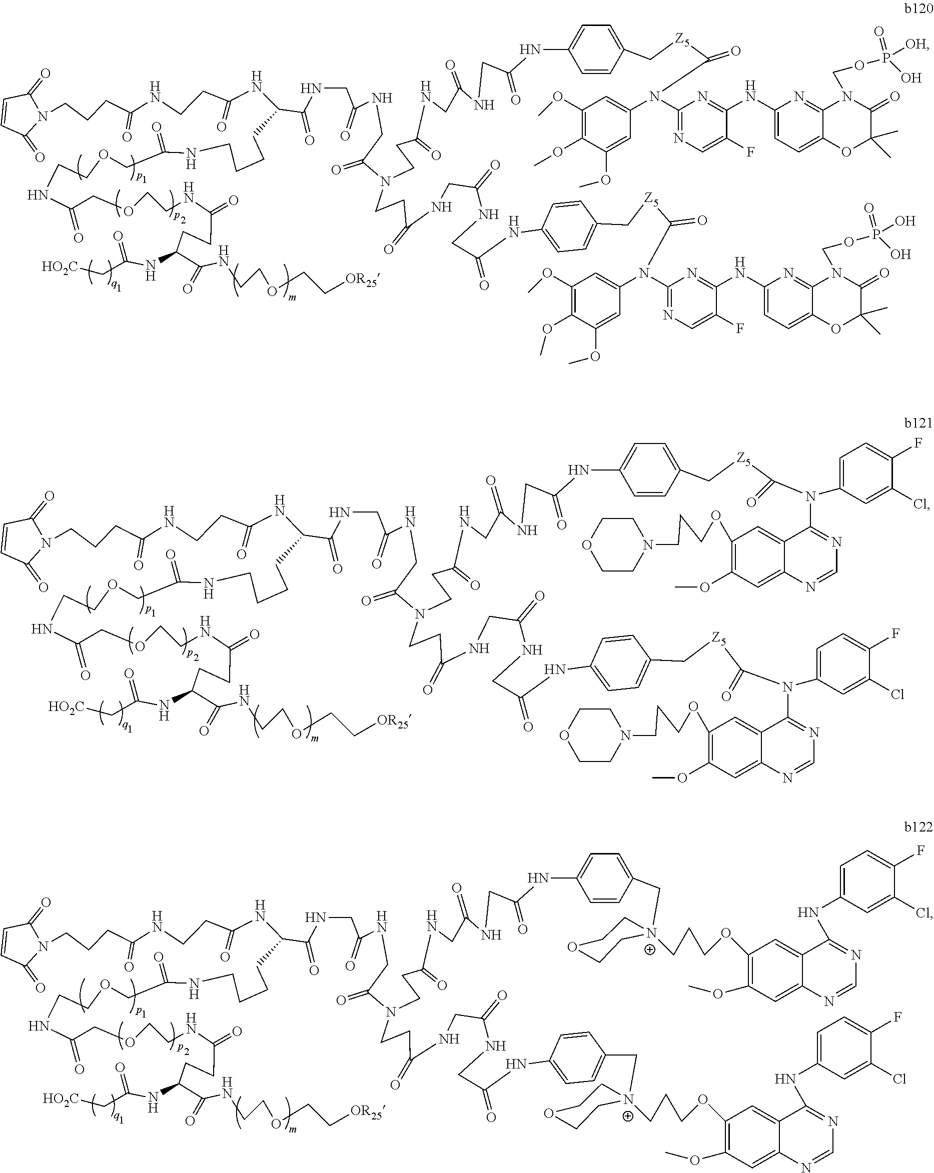

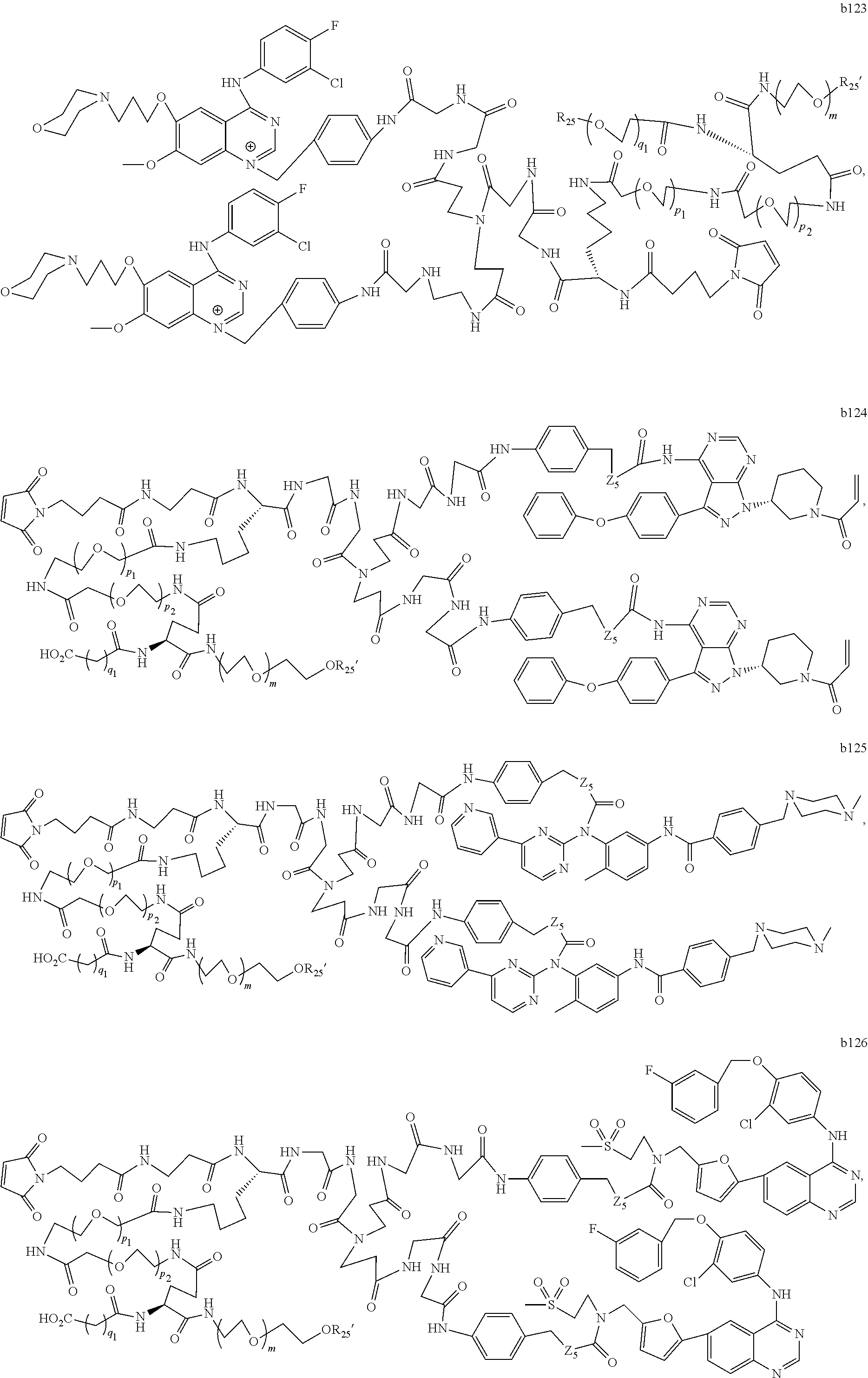

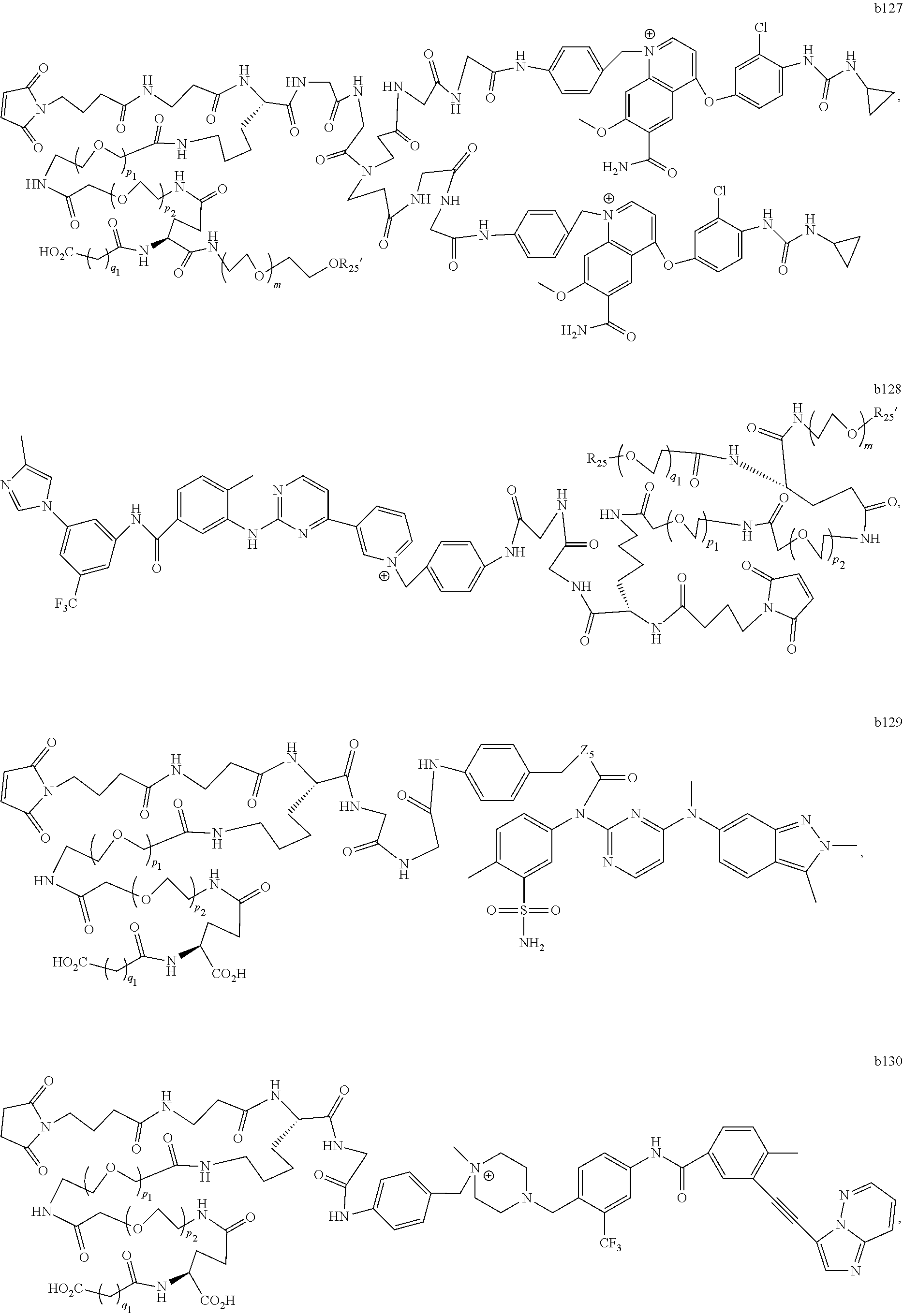

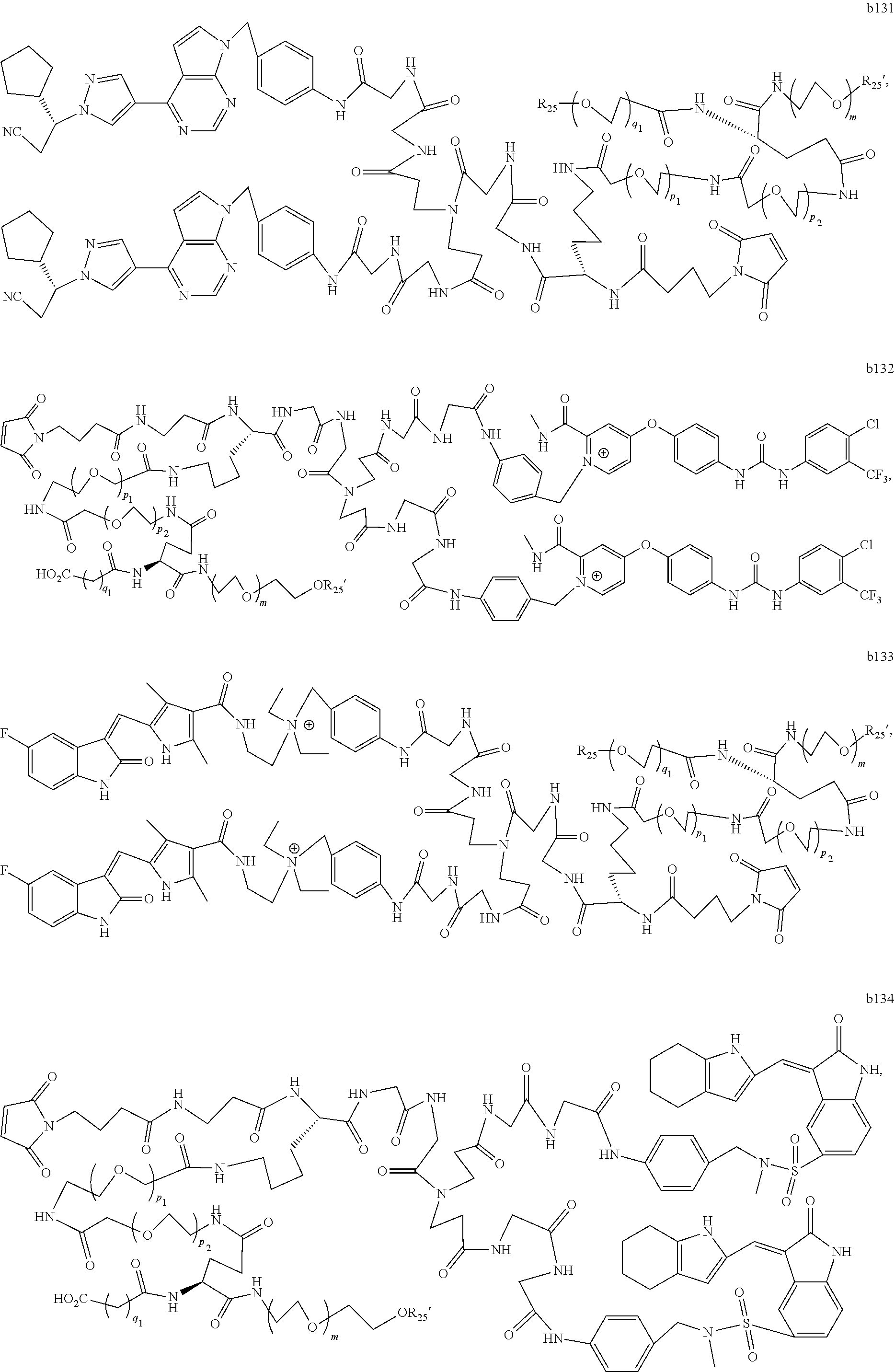

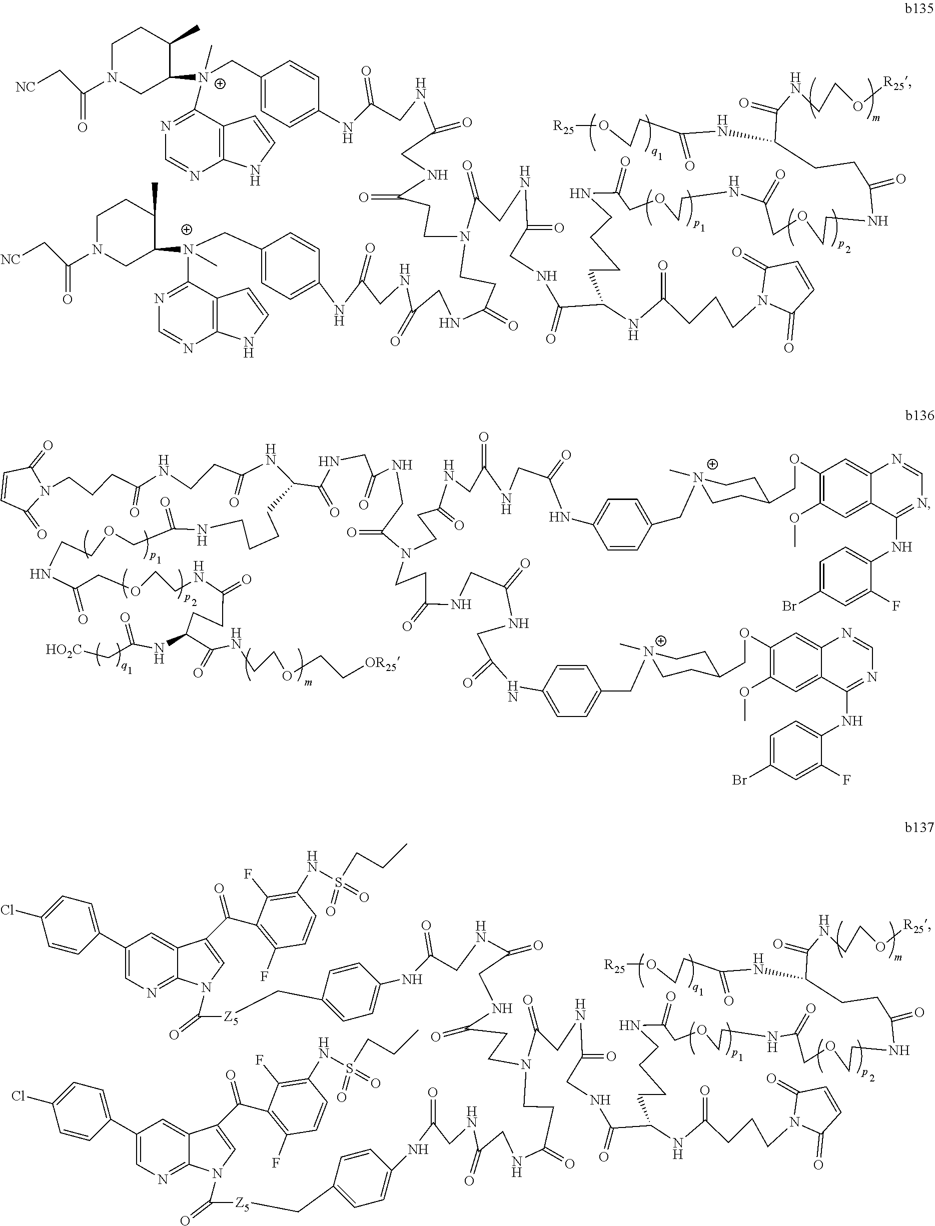

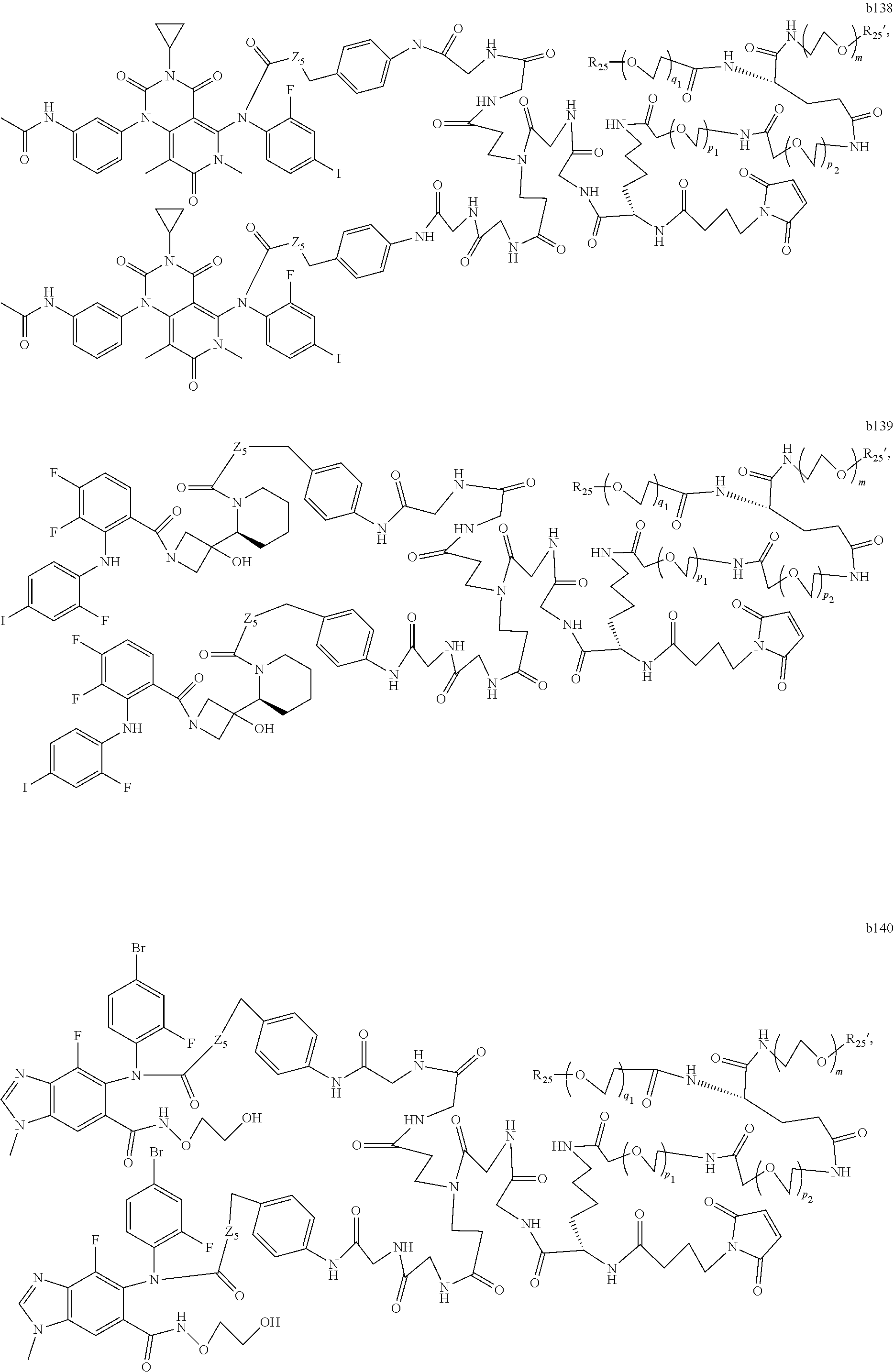

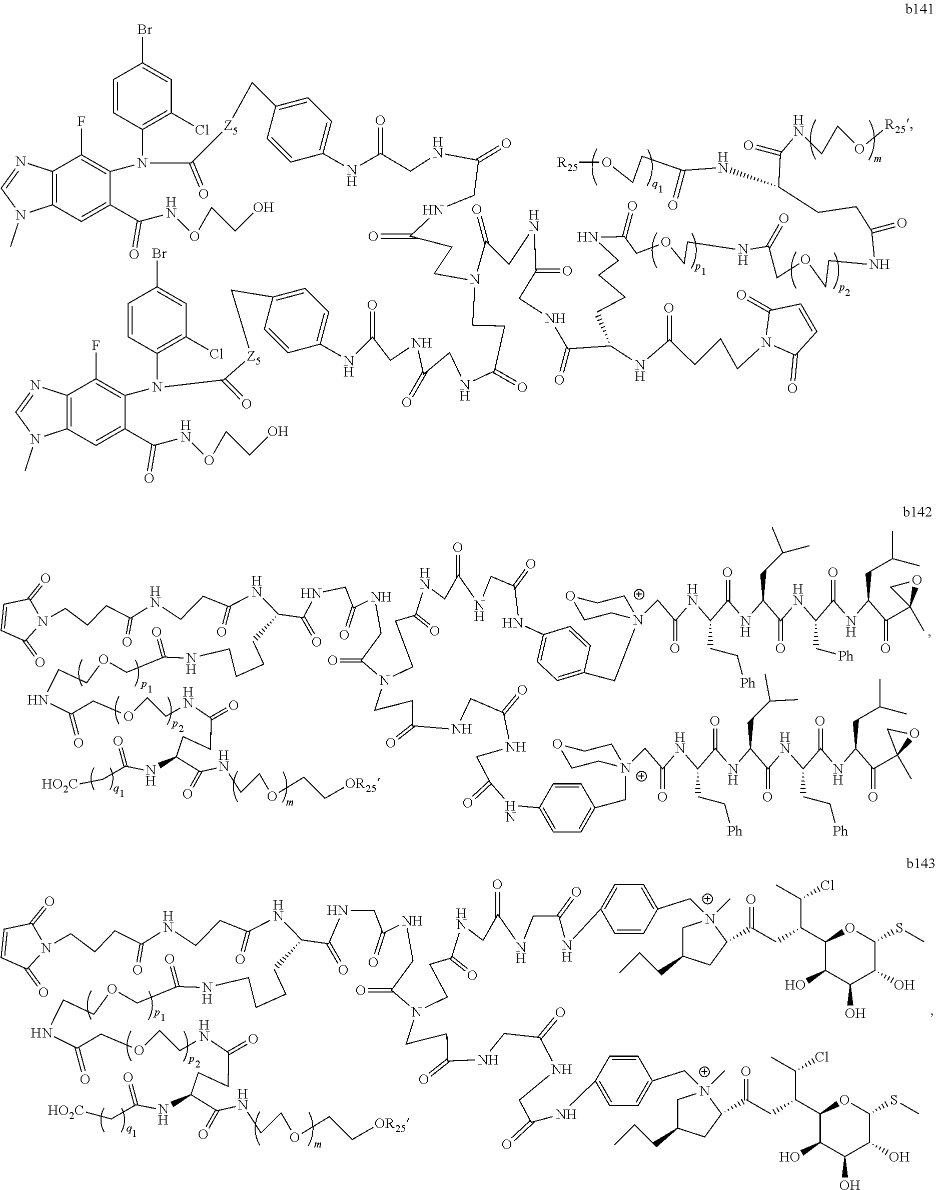

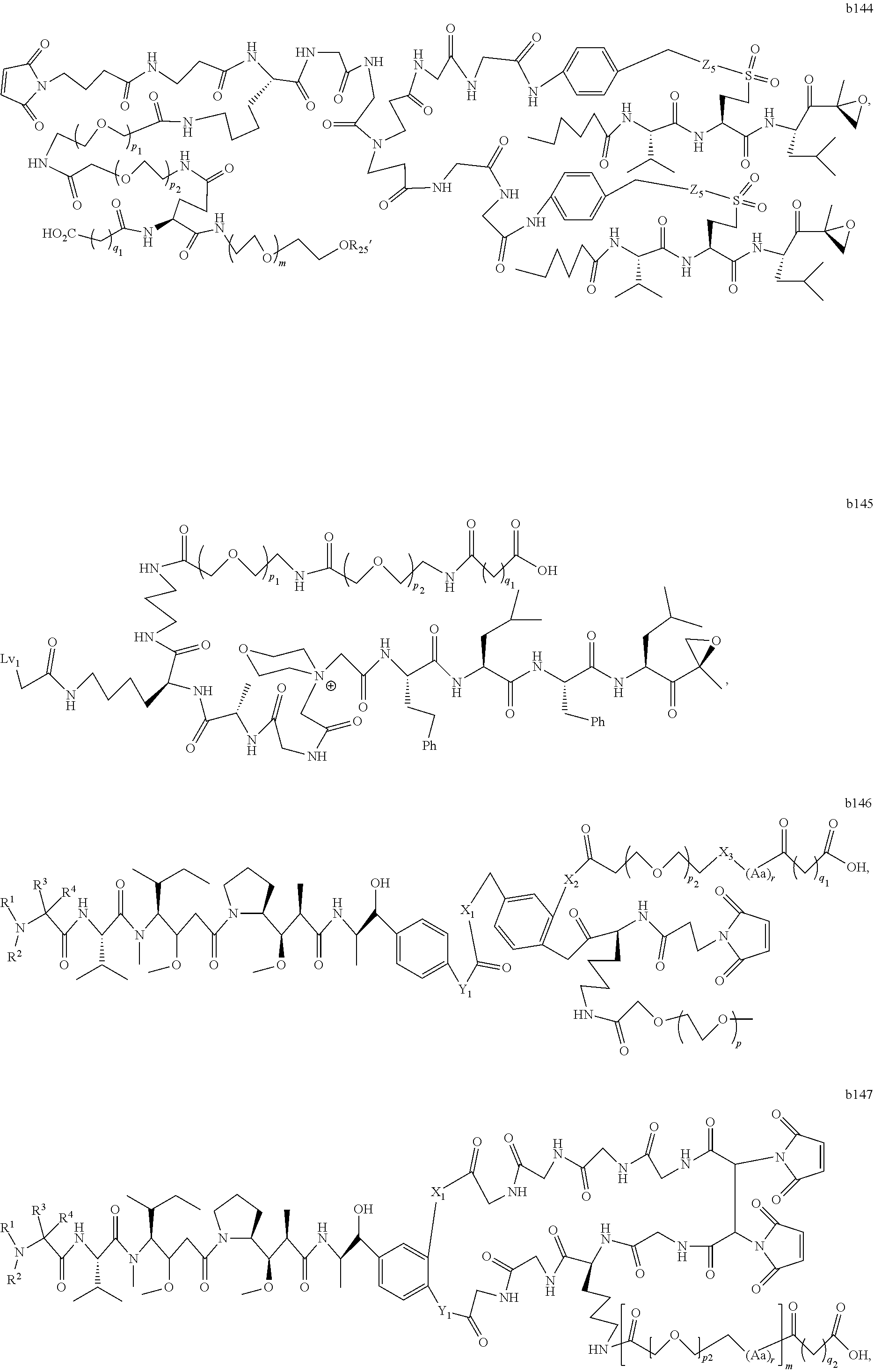

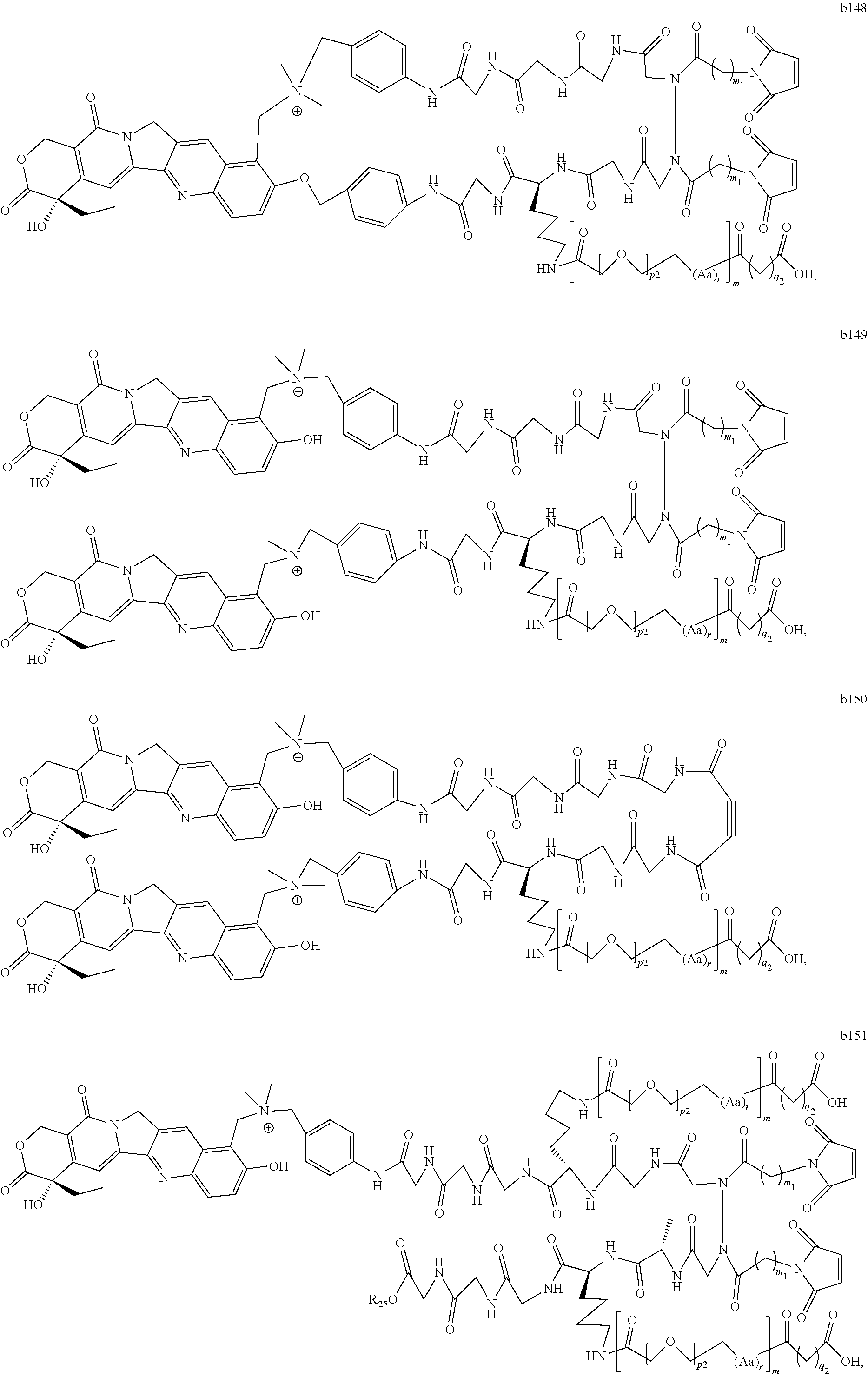

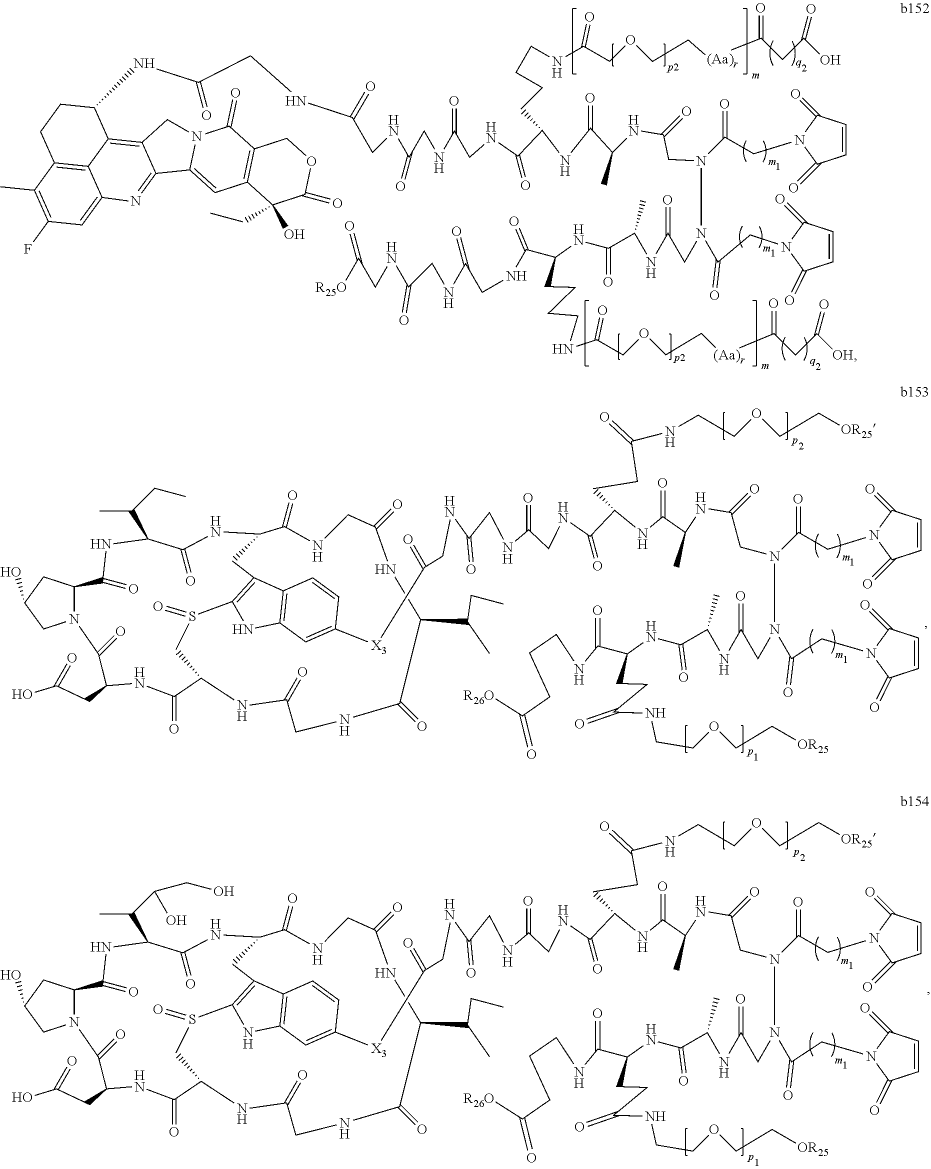

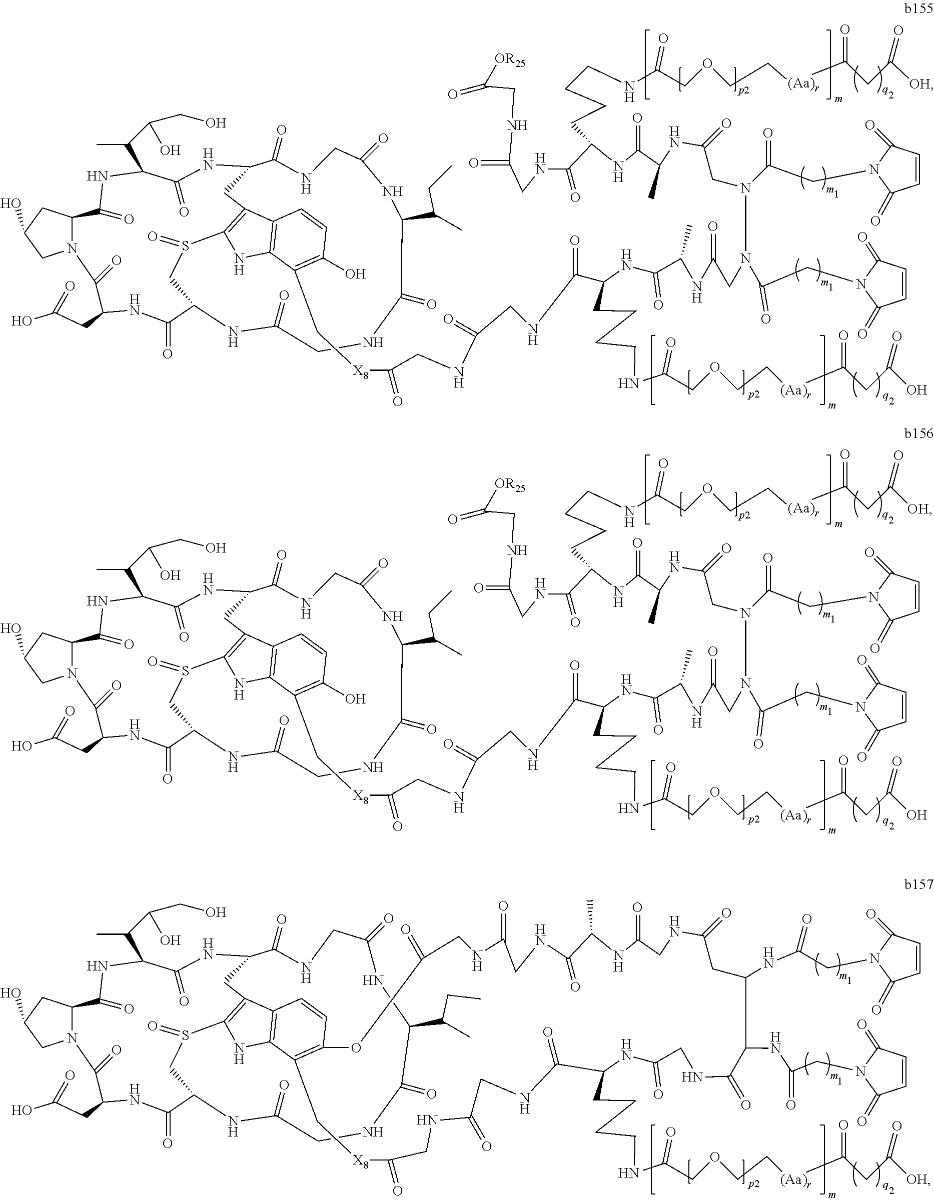

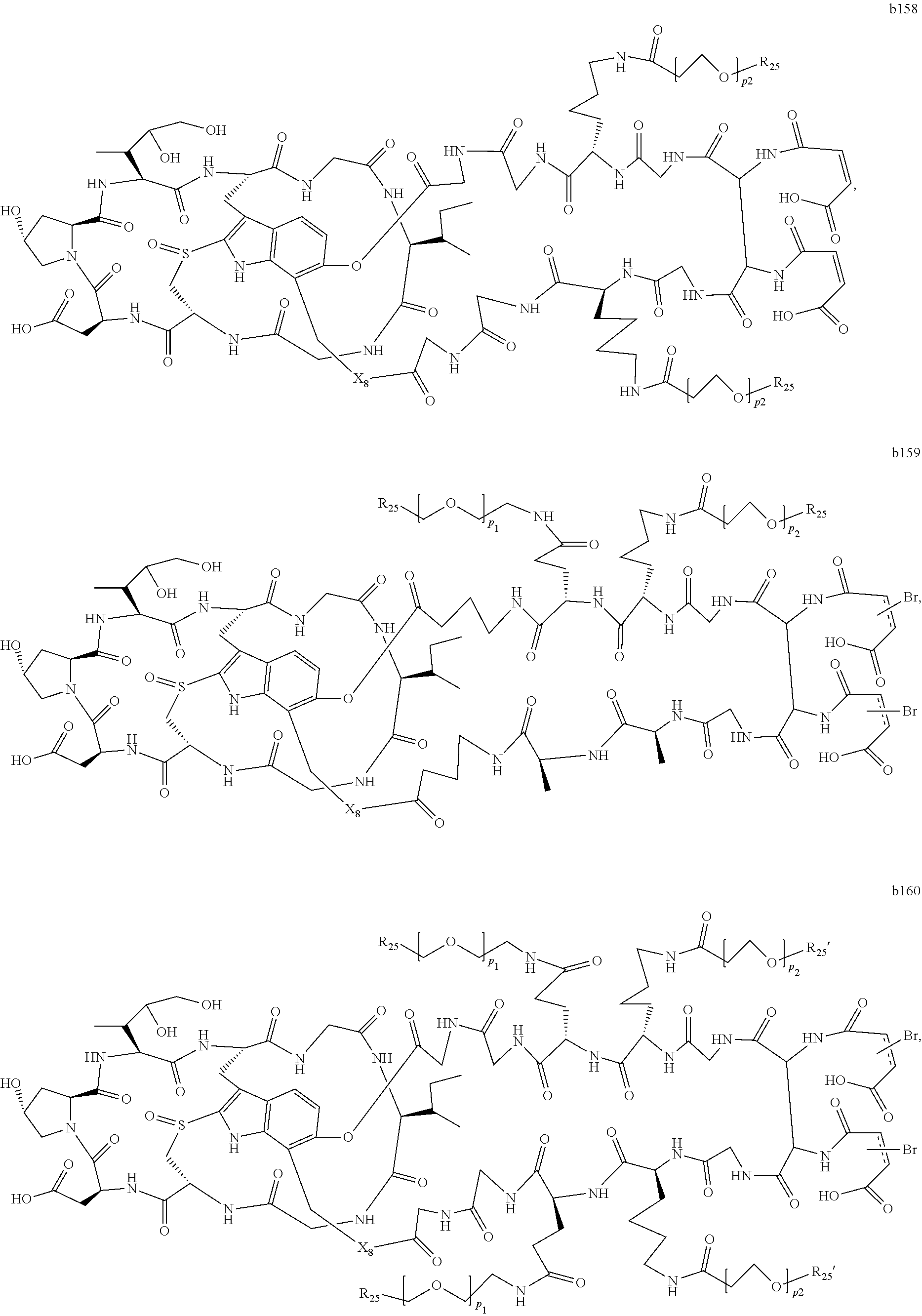

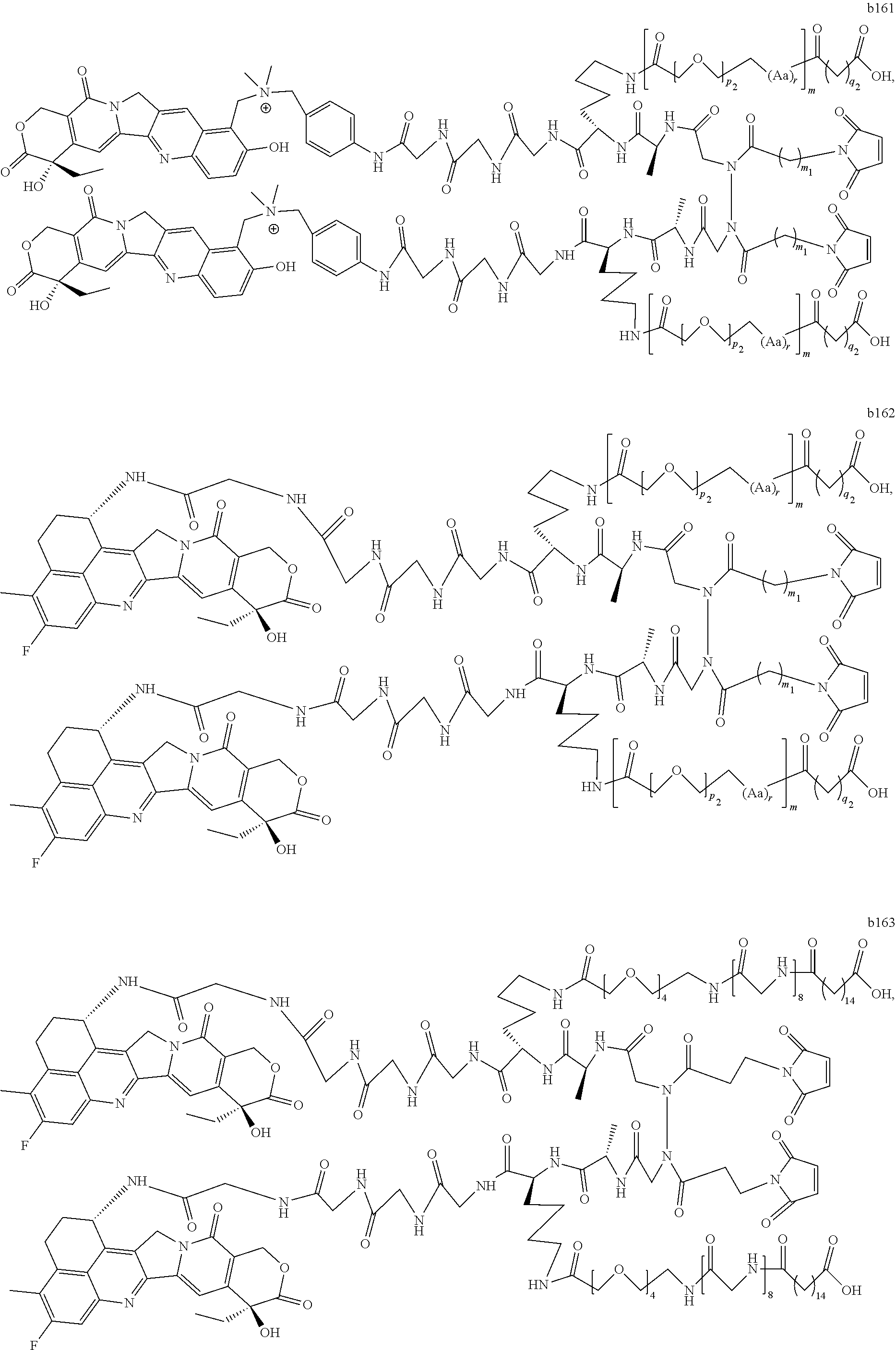

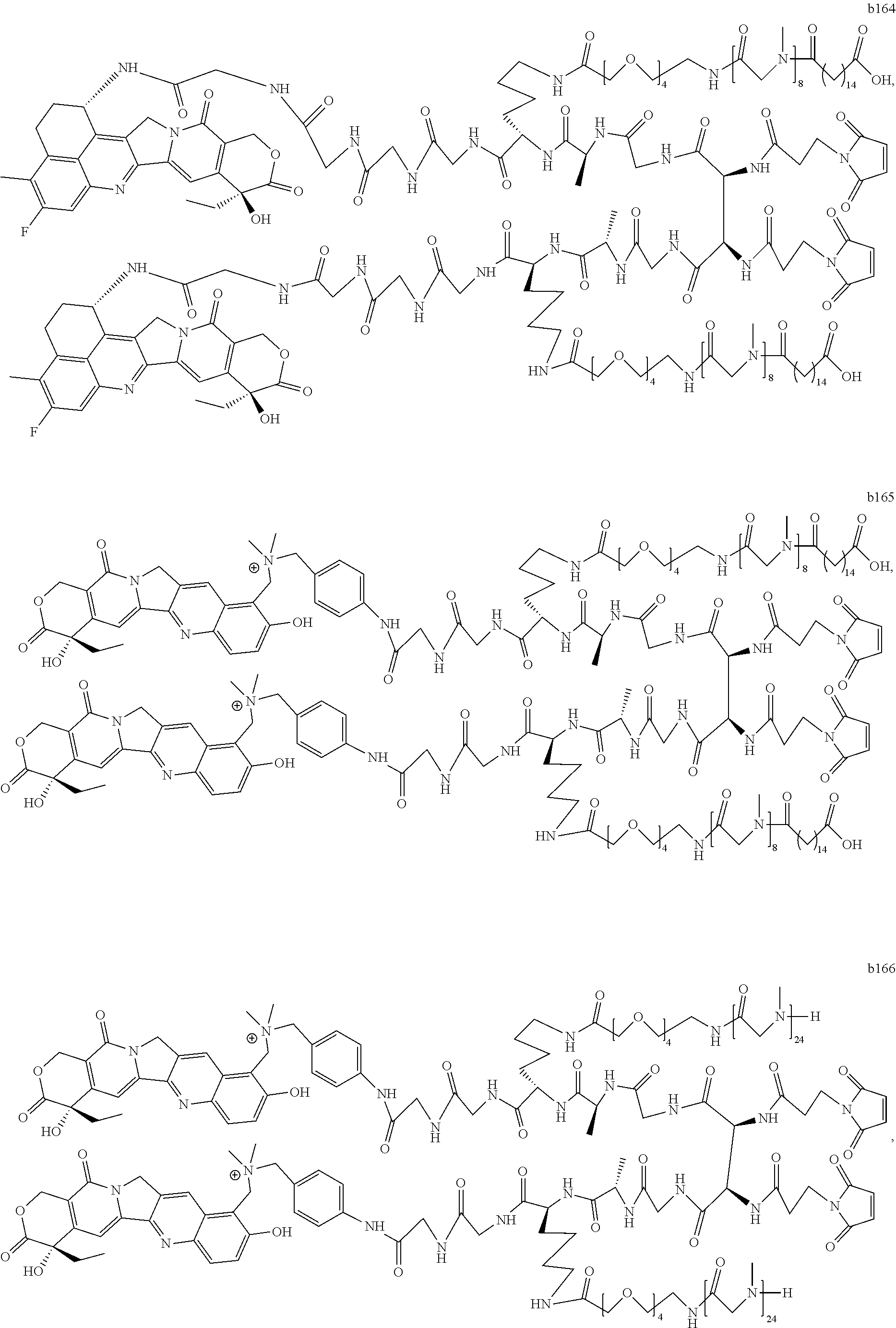

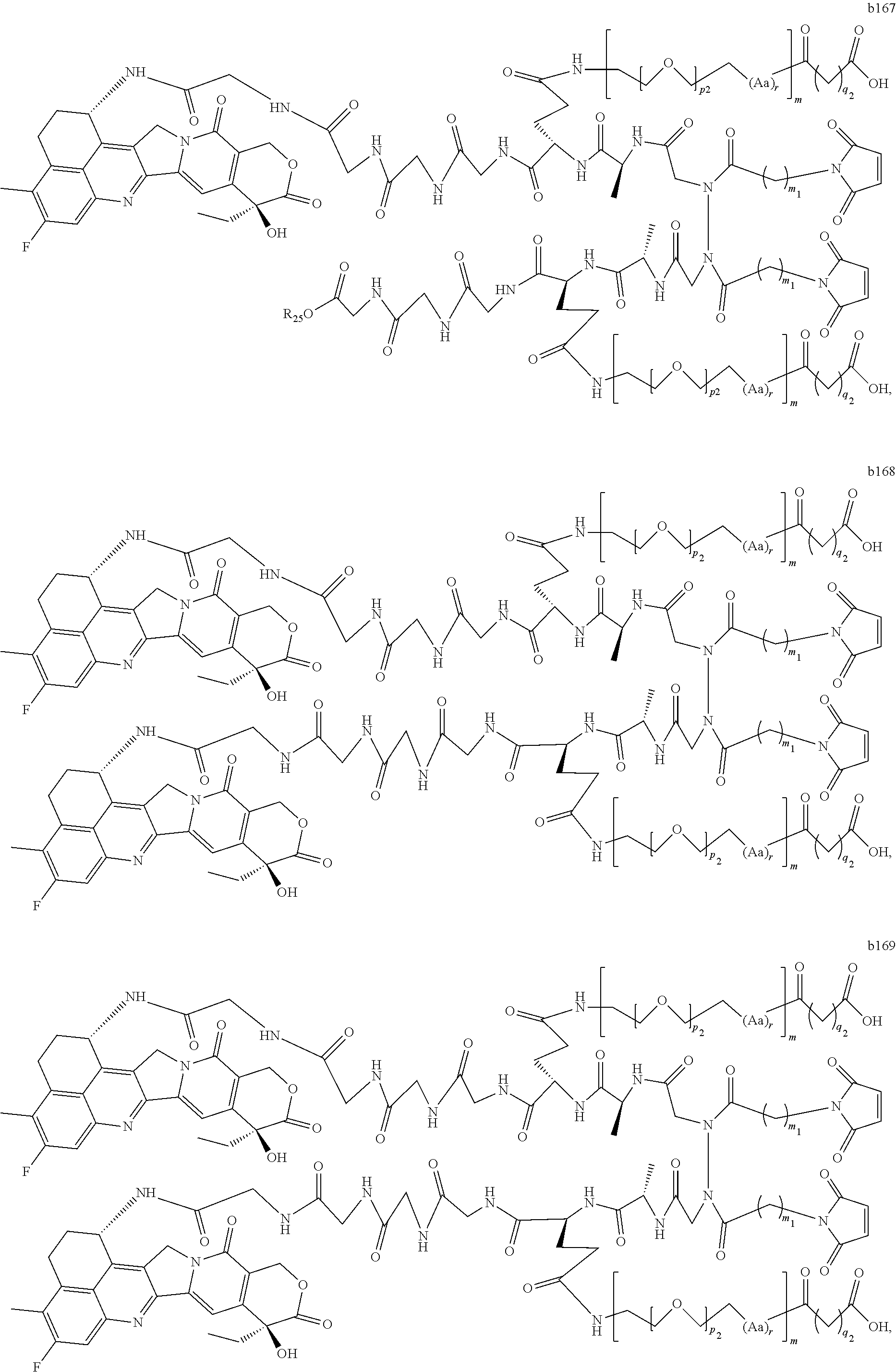

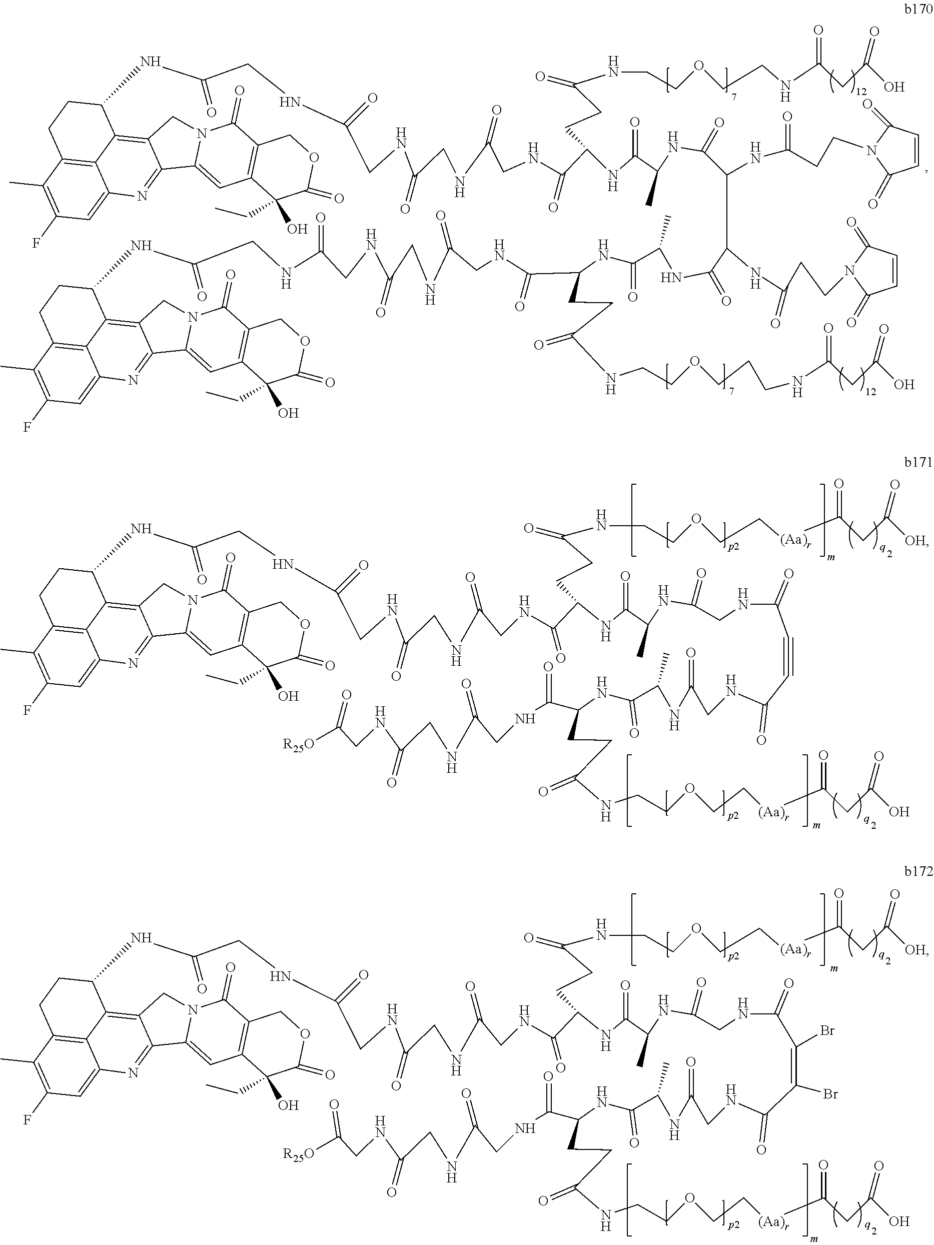

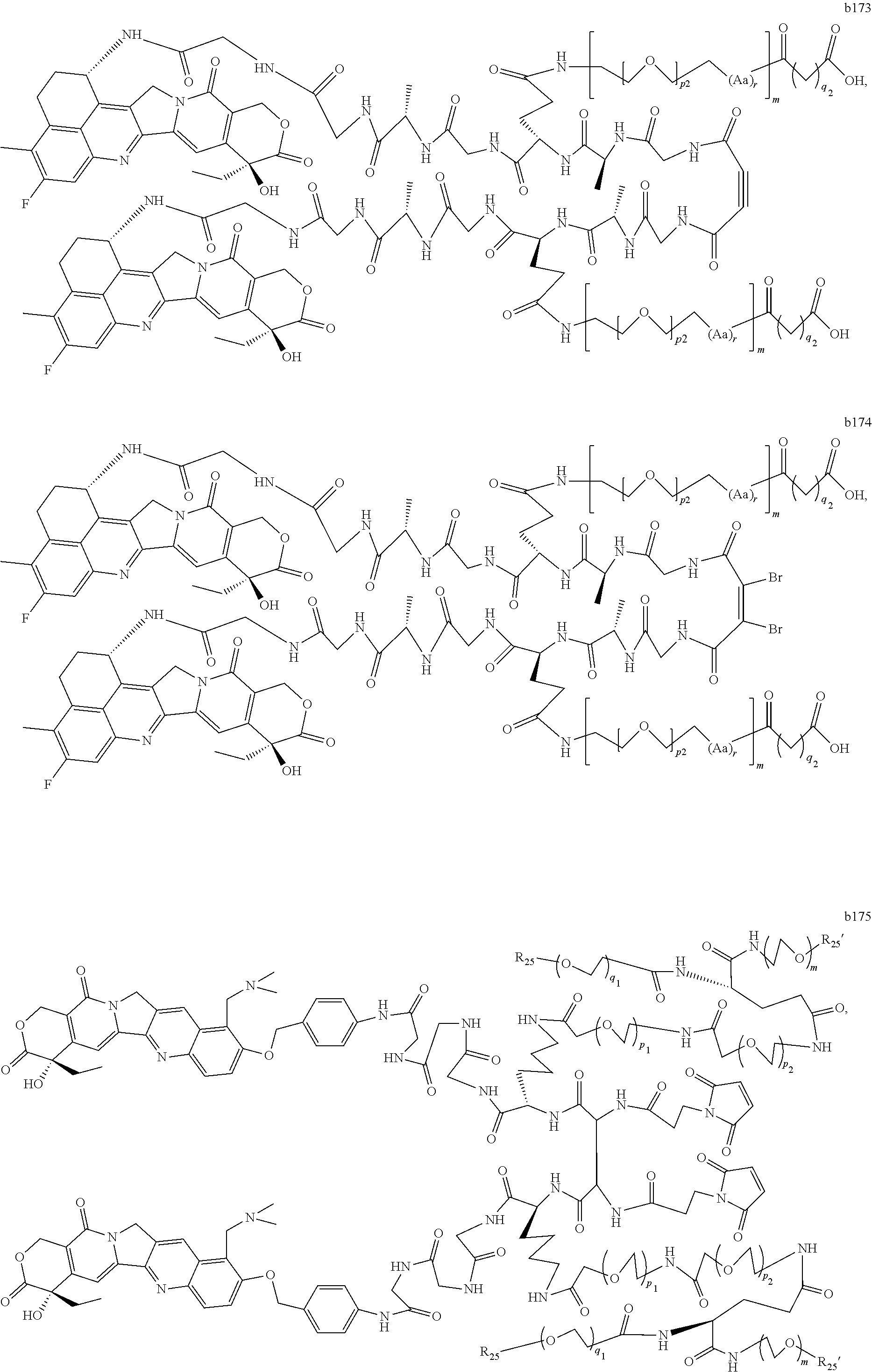

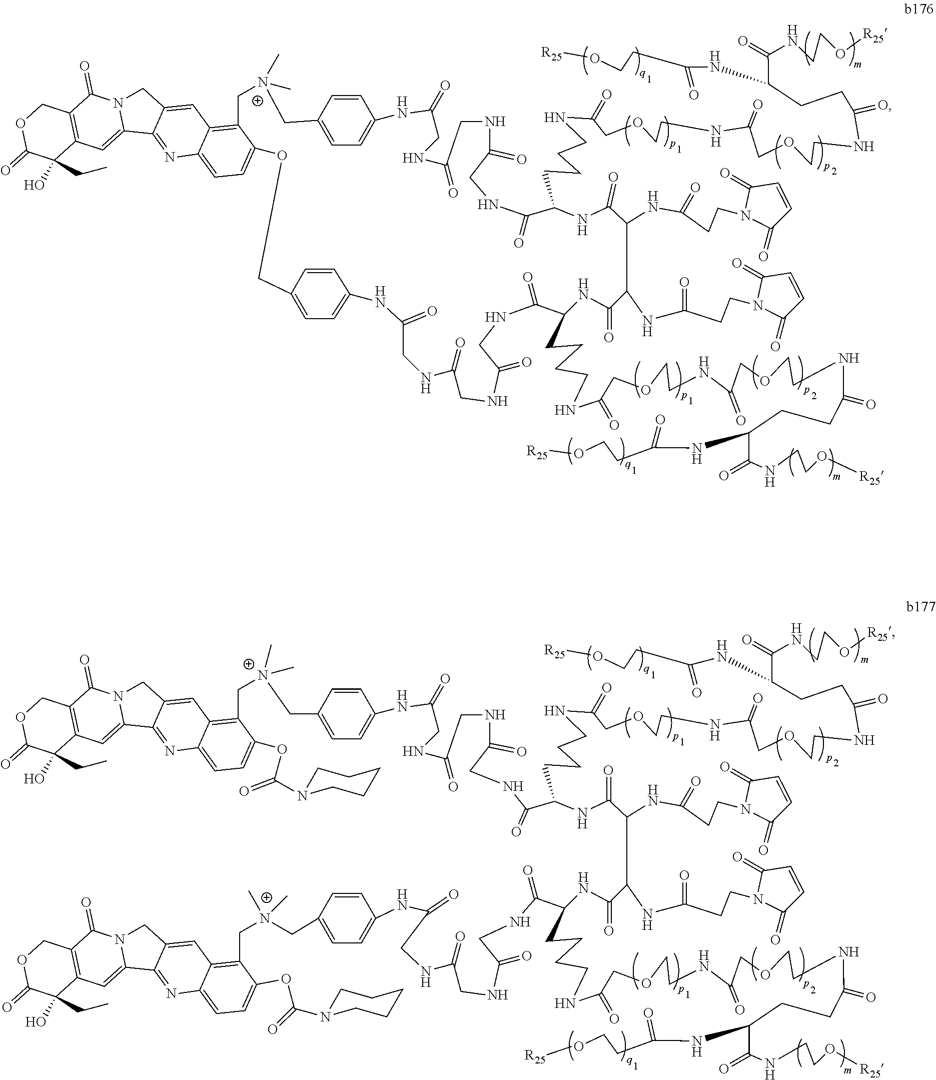

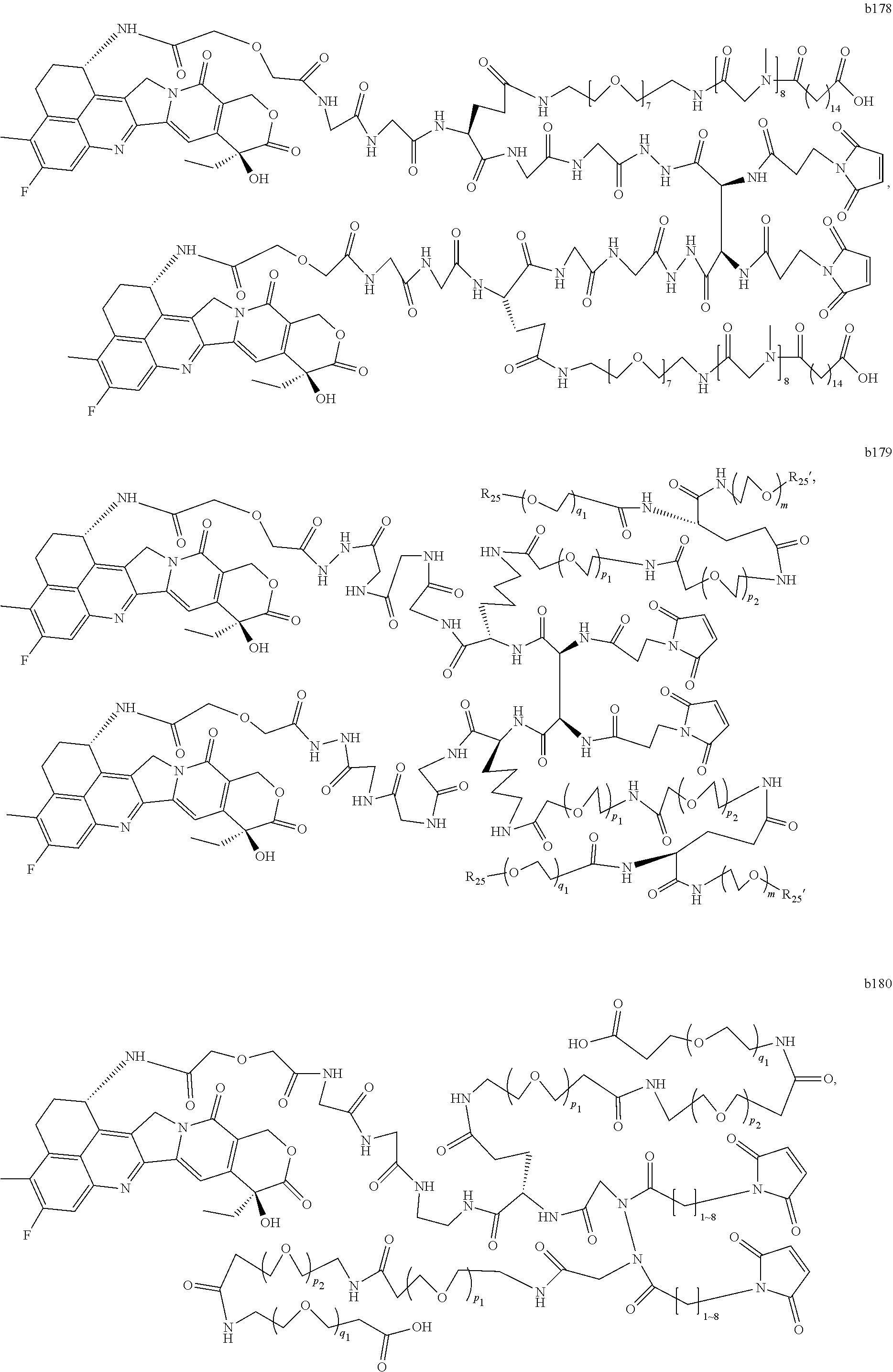

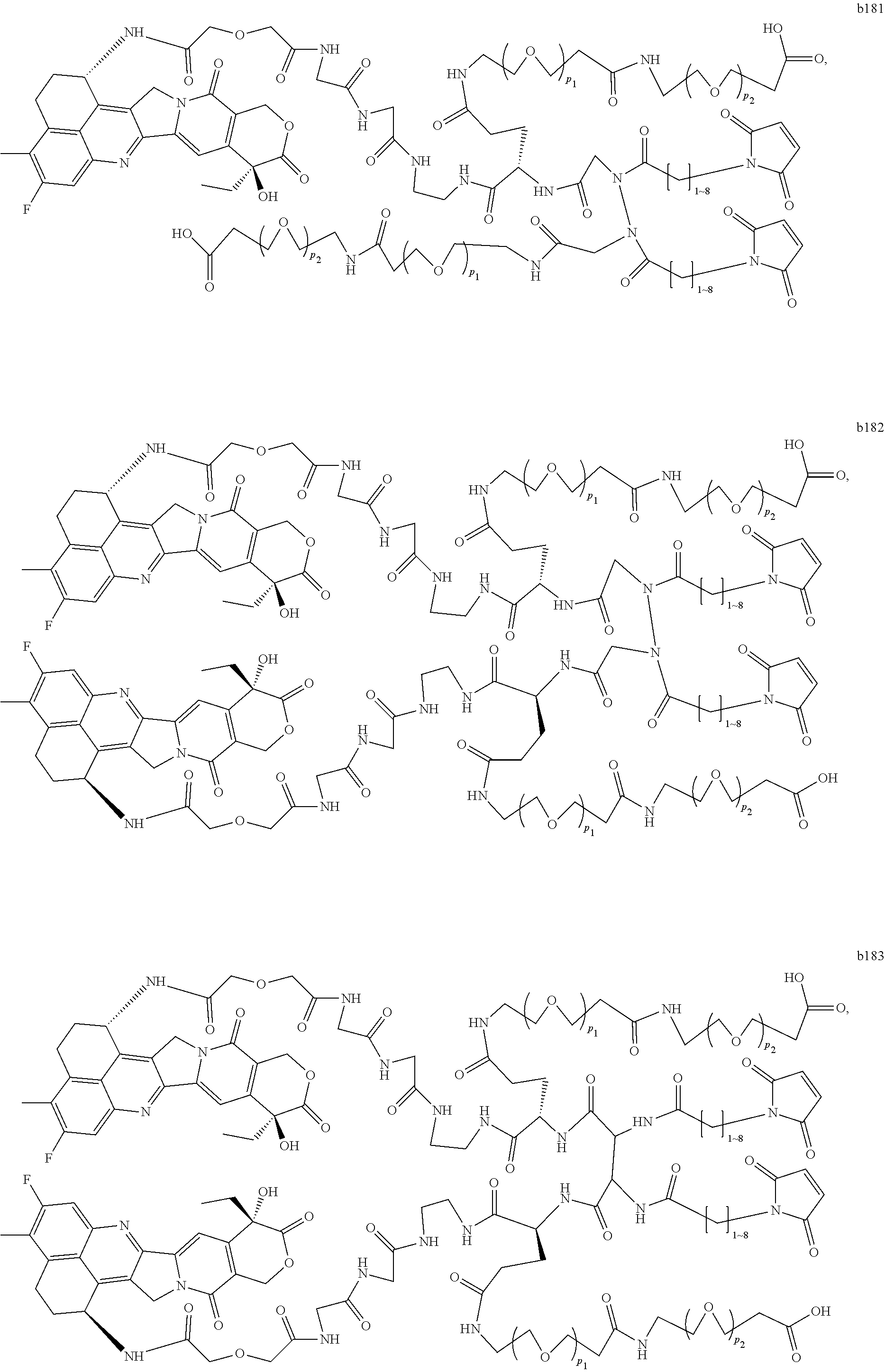

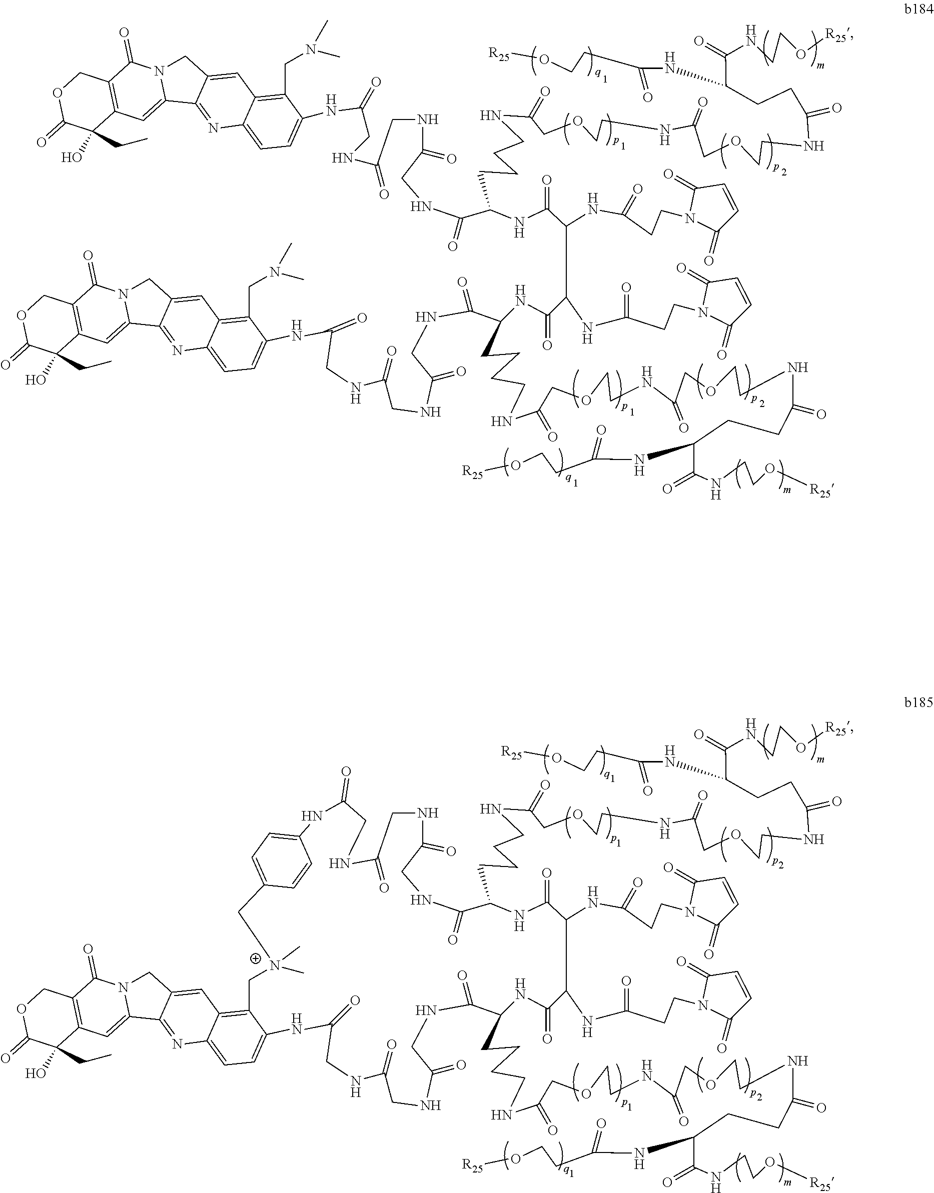

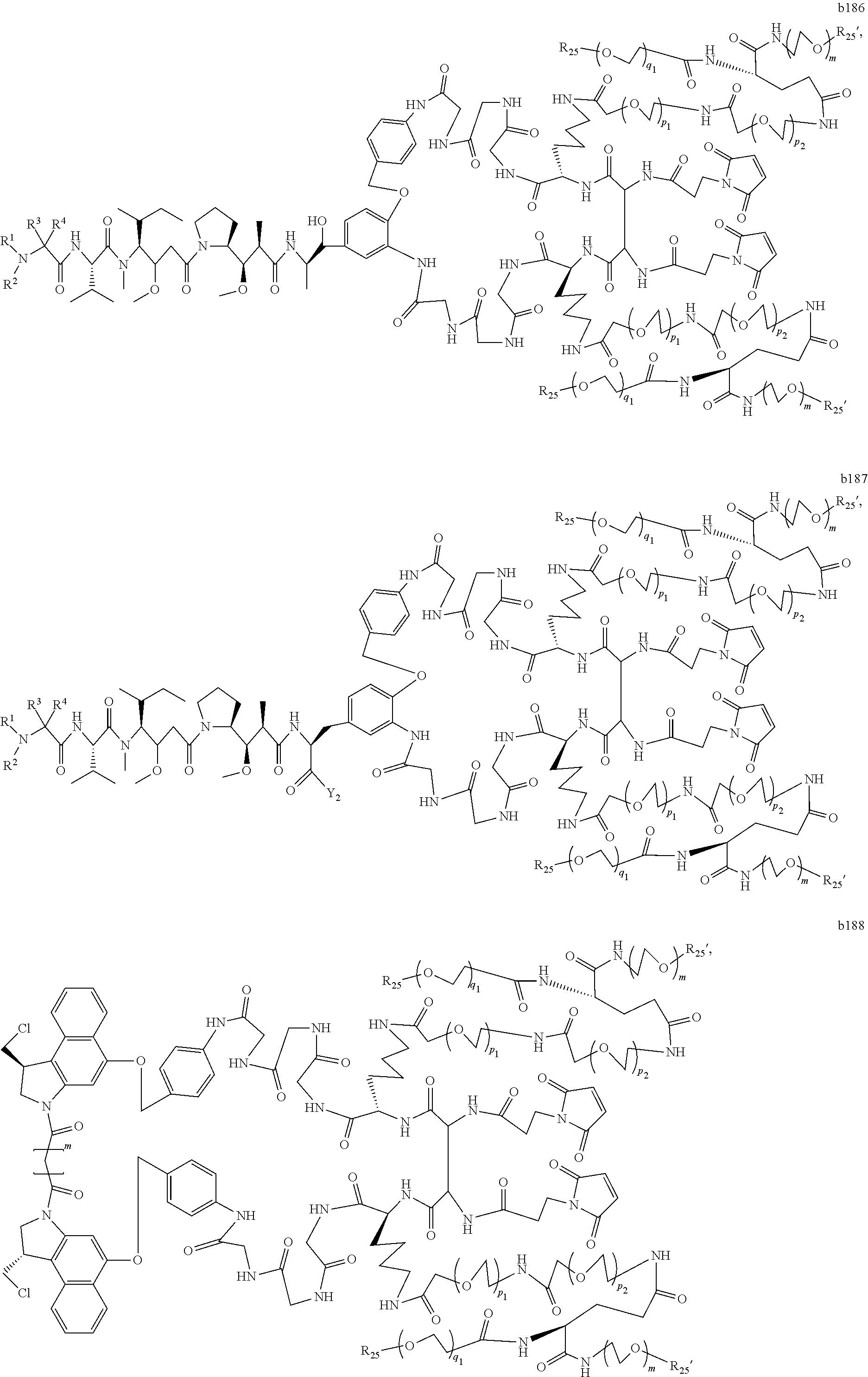

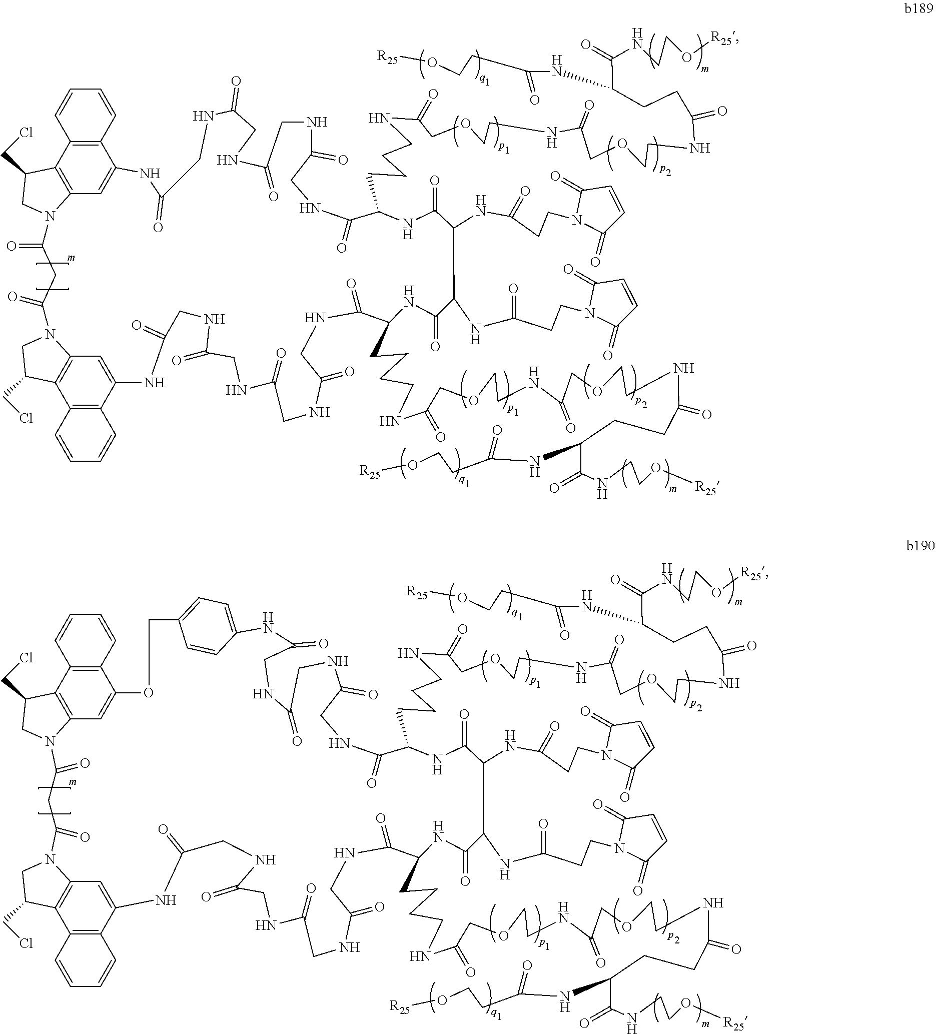

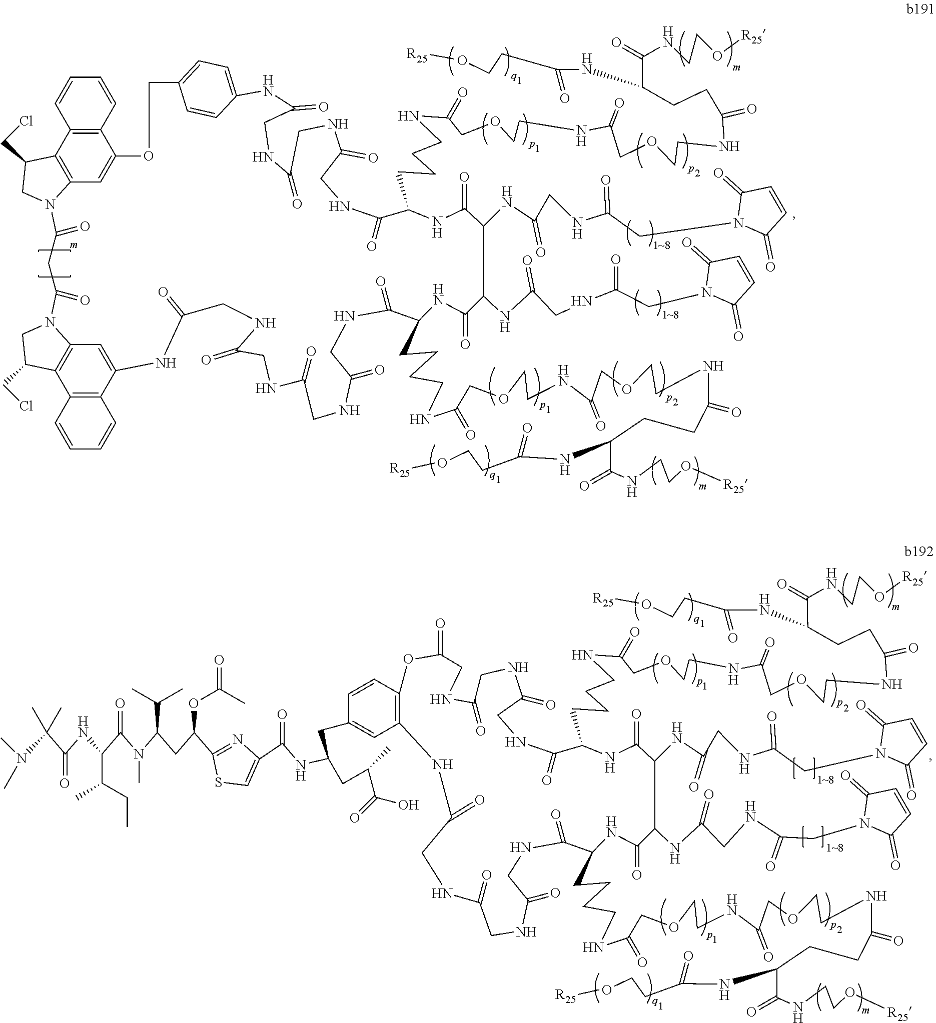

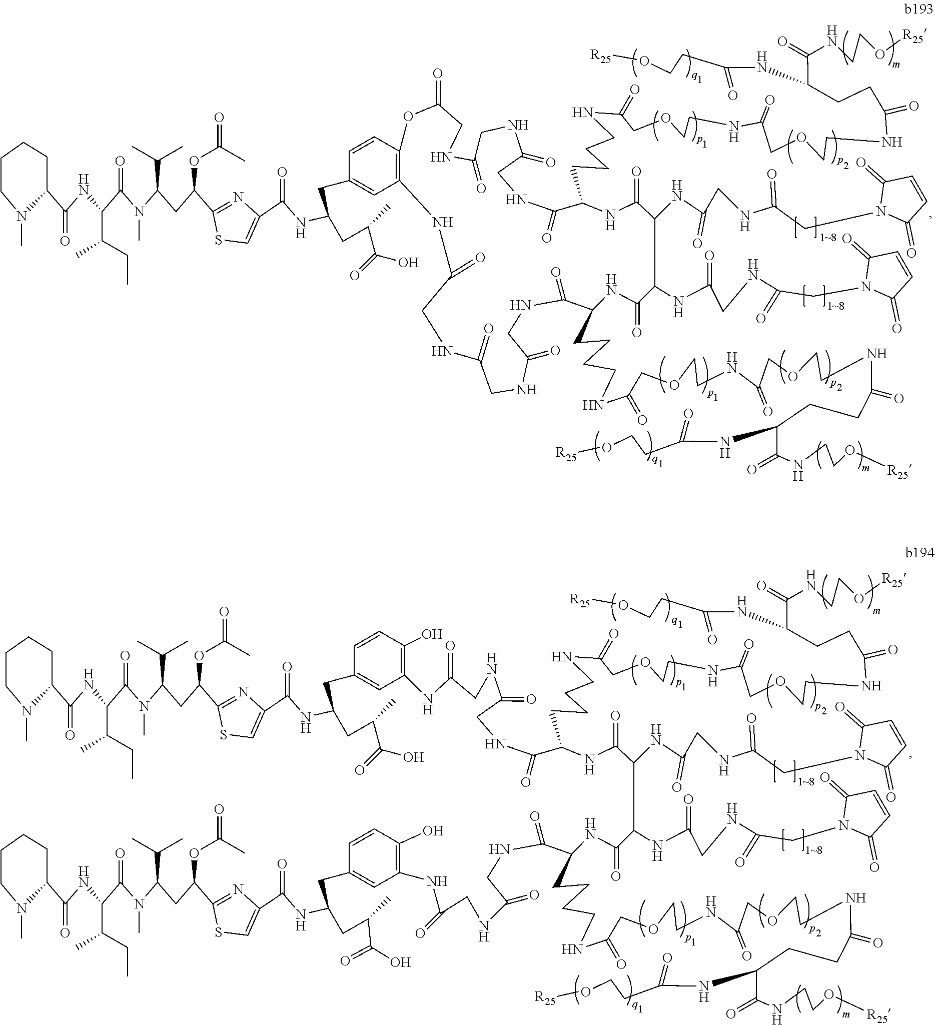

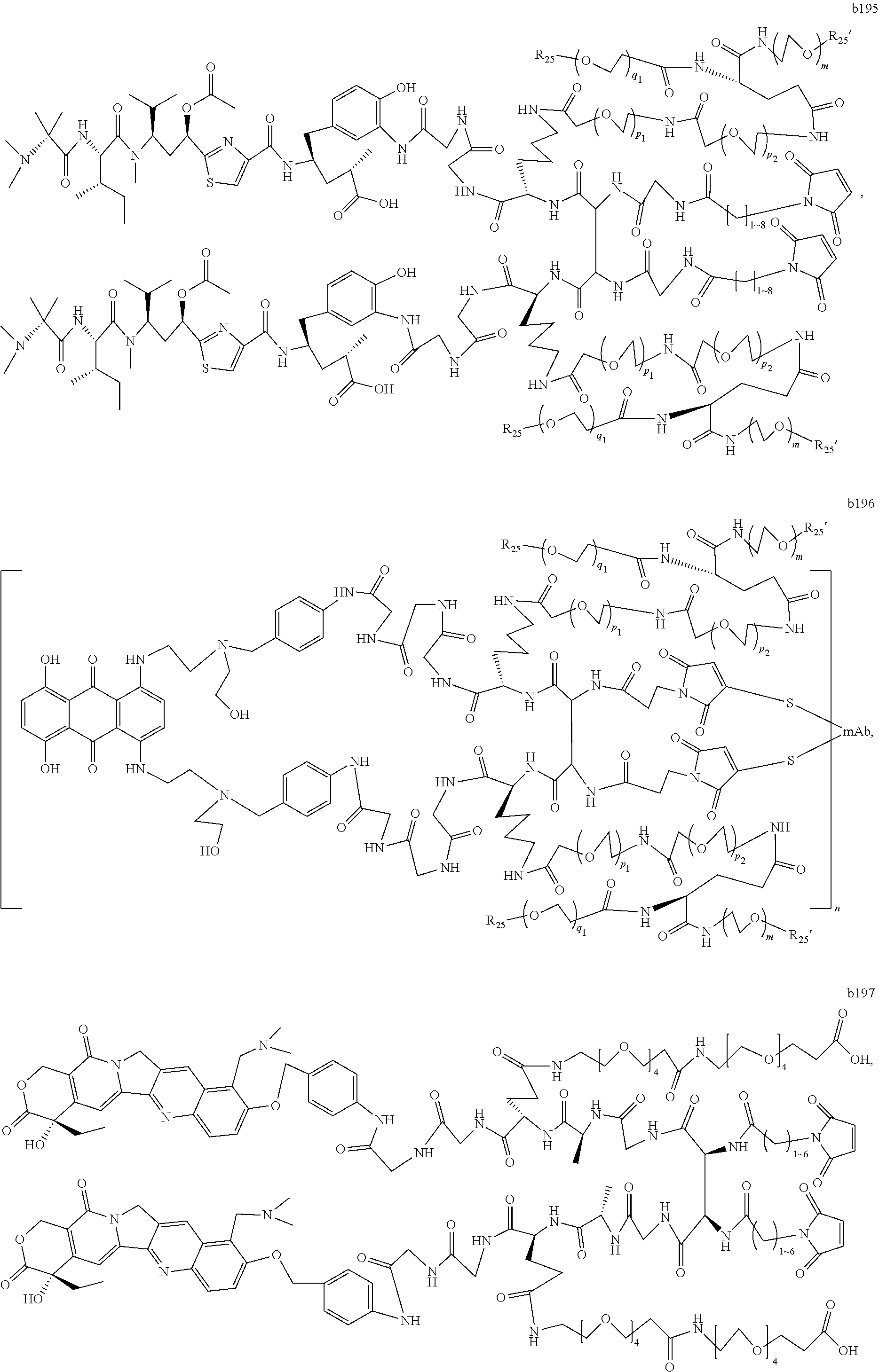

or one or more isotope of chemical elements, pharmaceutically acceptable salts, hydrates, or hydrated salts; or the polymorphic crystalline structures of these compounds; or the optical isomers, racemates, diastereomers or enantiomers; wherein X₈ is O, S, NH, NHNH, NHR₁₂, SR₁₂, SSR₁₂, SSCH(CH₃)R₁₂, SSC(CH₃)₂R₁₂, or R₁₂; R¹, R², R³, R⁴, R⁵, R₄, R₅, R₇, R₈, R₉, R₁₀, X₁, X₂, X₃, X₄, X₅, X₆, Y₁, Y₂, Y₃, Y₅, R₁₂, R_{12′}, R₁₃, R₁₃′, R₂₅, R₂₅′, Z₂, Z₃, p. p₁, p₂, p₃, q₁, q₂, Lv₁, Lv₂, Lv₃, Lv_{3′}, m, m₁, n, and mAb are described the same above; Aa is natural or unnatural amino acid; r is 0-12; (Aa)ᵣ is a peptide containing the same or different sequence of amino acids when r>2; r=0 means (Aa)r absent.

Preferably Lv₁, Lv₂, Lv₃ and Lv₃′ react to thiols of a cell-binding agent/molecule. The thiols are more preferably pairs of sulfur atoms reduced from the inter chain disulfide bonds of the cell-binding agent by a reducing agent selected from dithiothreitol (DTT), dithioerythritol (DTE), L-glutathione (GSH), tris (2-carboxyethyl) phosphine (TCEP), 2-mercaptoethylamine (β-MEA), or/and beta mercaptoethanol (β-ME, 2-ME). The thiol of a cell-binding agent/molecule can also be generated through Traut's reagent or a thiolactone, wherein the Traut's reagent or a thiolactone react to an amine of the cell-binding agent/molecule to form a thiol, following by simultaneously or sequentially react to Lv₁, Lv₂, Lv₃ or Lv_{3′}.

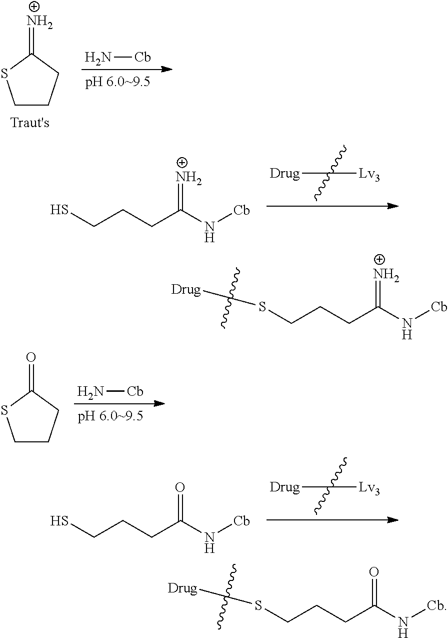

The present invention further relates to a method of making a cell-binding molecule-amatoxin analog conjugate of Formula (I), (II) and (III) as well the application of the conjugates of Formula (I), (II) and (III).

The Preparation of a Conjugate of a Drug to a Cell Binding Molecule Via a Side Chain-Linkage

The preparation of the conjugates of an amatoxin analog to a cell binding molecules of the present invention and the synthetic routes to produce the conjugates via side chain-linkage are shown in FIGS. 1˜19 and in the experimental section.

The conjugates of Formula (I), (II) and (III) can be prepared through the intermediate compounds of Formula (IV), (V) and (VI) respectively. In general, the compounds of Formula (IV), (V) and (VI) are synthesized to have the function groups of maleimido, Lv₁ and Lv₂ that can be readily reacted to a cell-binding molecule or to a modified cell-binding molecule. The synthesis of the compounds of Formula (IV), (V) and (VI) and some of preparations of Formula (I), (II) and (III) are structurally shown in the FIGS. 1˜19.

To synthesize the conjugate of Formula (I), in general, a function group Lv₁ on Formula (IV) reacts one, two or more residues of a cell binding molecule at 0-60° C., pH 5˜ 9 aqueous media with or without addition of 0˜30% of water mixable (miscible) organic solvents, such as DMA, DMF, ethanol, methanol, acetone, acetonitrile, THF, isopropanol, dioxane, propylene glycol, or ethylene diol, following by dialysis or chromatographic purification to form a conjugate compound of Formula (I). Some of the residue (reacting group for conjugation) of the cell-binding molecule can be obtained through protein engineering.

The conjugates of the Formula (II) and (III) can also be obtained through the reaction of the function group Lv₁, and Lv₂ of linkers of the Formula (V) and (VI) to two or more residues of a cell binding molecule, preferably a pair of free thiols generated through reduction of disulfide bonds of the cell-binding molecule at 0-60° C., pH 5˜9 aqueous media with or without addition of 0˜30% of water mixable (miscible) organic solvents, to form the conjugate molecule. The pairs of thiols are preferred pairs of disulfide bonds reduced from the inter chain disulfide bonds of the cell-binding agent by a reducing agent which can selected from dithiothreitol (DTT), dithioerythritol (DTE), L-glutathione (GSH), tris (2-carboxyethyl) phosphine (TCEP), 2-mercaptoethylamine (P-MEA), or/and beta mercaptoethanol (β-ME, 2-ME) at pH 4˜9 aqueous media with or without addition of 0˜30% of water mixable (miscible) organic solvents.

The reactive groups of Lv₁, and Lv₂ on Formula (IV) (V) and (VI), which can be independently disulfide, thiol, thioester, maleimido, halogen substituted maleimides, haloacetyl, azide, 1-yne, ketone, aldehyde, alkoxyamino, triflate, carbonylimidazole, tosylate, mesylate, 2-ethyl-5-phenylisoxazolium-3′-sulfonate, or carboxyl acid esters of nitrophenol, N-hydroxysuccinimide (NHS), phenol; dinitrophenol, pentafluorophenol, tetrafluorophenol, difluorophenol, monofluorophenol, pentachlorophenol, dichlorophenol, tetrachlorophenol, 1-hydroxybenzotriazole, anhydrides, or hydrazide groups, or other acid ester derivatives, can react to one, two or more groups on a cell-binding molecule/agent, simultaneously or sequentially at 0-60° C., pH 4˜9.5 aqueous media with or without addition of 0˜30% of water mixable (miscible) organic solvents, to yield a conjugate of the Formula (I), (II) and (III), after column purification or dialysis. The reactive groups of Lv₁ and Lv₂ on Formula (IV), (V) and Formula (VI) react to the modified cell-binding molecule in different ways accordingly. For example, a linkage containing disulfide bonds in a cell-binding agent-amatoxin analog conjugate of Formula (I) is achieved by a disulfide exchange between the disulfide bond in the modified cell-binding agent and Lv₁ and Lv₂ having a free thiol group, or by a disulfide exchange between a free thiol group in the modified cell-binding agent and a disulfide bond on Lv₁ and/or Lv₂. In order to swift the disulfide exchange reaction, the disulfide group normally are a group of disulfanylpyridine, disulfanyl-nitropyridine, disulfanyl-nitrobenzene, disulfanyl-nitrobenzoic acid, or disulfanyl-dinitrobenzene, etc. A linkage containing thioether bonds in the conjugates of Formula (I) (II) and (III) is achieved by reaction of the maleimido or haloacetyl or ethylsulfonyl either on a modified cell-binding agent or a compound of Formula (IV), (V) and (VI) to a free thiol group on a compound of Formula (IV), (V) and Formula (VI) or on a modified cell-binding agent respectively; A linkage containing a bond of an acid labile hydrazone in the conjugates can be achieved by reaction of a carbonyl group of the drug of Formula (IV), (V) and (VI) or of cell-binding molecule with the hydrazide moiety on a modified cell-binding molecule or on the drug of Formula (IV), (V) and (VI) accordingly, by methods known in the art (see, for example, P. Hamann et al., Cancer Res. 53, 3336-34, 1993; B. Laguzza et al., J. Med. Chem., 32; 548-55, 1959; P. Trail et al., Cancer Res., 57; 100-5, 1997); A linkage containing a bond of triazole in the conjugates can be achieved by reaction of a 1-yne group of the drug of Formula (IV), (V) and (VI) or of cell-binding molecule with the azido moiety on the other counter part accordingly, through the click chemistry (Huisgen cycloaddition) (Lutz, J-F. et al, 2008, Adv. Drug Del. Rev. 60, 958-70; Sletten, E. M. et al 2011, AccChem. Research 44, 666-76). A linkage containing a bond of oxime in the conjugates linked via oxime is achieved by reaction of a group of a ketone or aldehyde group of the drug of Formula (IV), (V) and (VI) or of a cell-binding molecule with a group of oxyamine on the other counter part respectively. A thiol-containing cell-binding molecule can react with the drug molecule linker of of Formula (IV), (V) and (VI) bearing a maleimido, or a haloacetyl, or an ethylsulfonyl substituent at pH 5.5˜9.0 in aqueous buffer to give a thioether linkage conjugate of Formula (I), (II) and (III). A thiol-containing cell-binding molecule can undergo disulfide exchange with a drug linker of Formula (IV), (V) and (VI) bearing a pyridyldithio moiety to give a conjugate having a disulfide bond linkage. A cell-binding molecule bearing a hydroxyl group or a thiol group can be reacted with a drug linker of Formula (IV), (V) and (VI) bearing a halogen, particularly the alpha halide of carboxylates, in the presence of a mild base, e.g. pH 8.0˜9.5, to give a modified drug bearing an ether or thiol ether linkage. A hydroxyl or an amino group on a cell-binding molecule can be condensed with a cross drug linker of Formula (IV), (V) and (VI) bearing a carboxyl group, in the presence of a dehydrating agent, such as EDC or DCC, to give ester linkage. A cell-binding molecule containing an amino group can condensate with a group of carboxyl ester of NHS, imidazole, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxyben-zotriazole; tosylate; mesylate; or 2-ethyl-5-phenylisoxazolium-3′-sulfonate on the drug-linker of Formula (IV), (V) and (VI) to give a conjugate via amide bond linkage.

The synthetic conjugate may be purified by standard biochemical means, such as gel filtration on a Sephadex G25 or Sephacryl S300 column, adsorption chromatography, and ion exchange or by dialysis. In some cases, a small molecule as a cell-binding agent (e.g. folic acid, melanocyte stimulating hormone, EGF etc) conjugated with a small molecular drugs can be purified by chromatography such as by HPLC, medium pressure column chromatography or ion exchange chromatography.

In order to achieve a higher yield of conjugation reaction for the Formula (I), (II) or (III) with a pair of free thiols on the cell-binding molecule, preferably on an antibody, a small percentage of water miscible organic solvents, or phase transfer agents, may be required to add to the reaction mixture. To cross-linking reagent (linker) of Formula (IV), (V) or (VI) can be first dissolved in a polar organic solvent that is miscible with water, for example in different alcohols, such as methanol, ethanol, and propanol, acetone, acetonitrile, tetrahydrofuran (THF), 1,4-dioxane, dimethyl formamide (DMF), dimethyl acetamide (DMA), or dimethylsulfoxide (DMSO) at a high concentration, for example 1-800 mM. Meanwhile, the cell-binding molecule, such as antibody dissolved in an aqueous buffer pH 4.0˜9.5, preferably pH 6.0˜8.5, at 1˜50 mg/ml concentration was treated with 0.5˜20 equivalent of TCEP or DTT for 20 min to 48 hour. After the reduction, DTT can be removed by SEC chromatographic purification. TCEP can be optionally removed by SEC chromatography too, or staying in the reaction mixture for the next step reaction without further purification, but preferably TCEP is neutralized with azide compounds, such as 4-azidobenzoic acid, 4-(azidomethyl)benzoic acid, or azido-polyethelene glycoyl (e. g. 2-(2-(2-(2-azidoethoxy)ethoxy)ethoxy)ethanol). Furthermore, the reduction of antibodies or the other cell-binding agents with TCEP can be performed along with existing a drug-linker molecule of Formula (IV), (V) or (VI), for which the cross-linking conjugation of the cell-binding molecules can be achieved simultaneously along with the TCEP reduction.

The aqueous solutions for the modification of cell-binding agents are buffered between pH 4 and 9, preferably between 6.0 and 8.0 and can contain any non-nucleophilic buffer salts useful for these pH ranges. Typical buffers include phosphate, acetate, triethanolamine HCl, HEPES, and MOPS buffers, which can contain additional components, such as cyclodextrins, hydroxypropyl-β-cyclodextrin, polyethylene glycols, sucrose and salts, for examples, NaCl and KCl. After the addition of the drug-linker of Formula (IV), (V) or (VI) into the solution containing the reduced cell-binding molecules, the reaction mixture is incubated at a temperature of from 0° C. to 50° C., preferably at 15° C.-37.5° C. The progress of the reaction can be monitored by measuring the decrease in the absorption at a certain UV wavelength, such as at 252 nm, or increase in the absorption at a certain UV wavelength, such as 280 nm, or the other appropriate wavelength. After the reaction is complete, isolation of the modified cell-binding agent can be performed in a routine way, using for example a gel filtration chromatography, an ion exchange chromatography, an adsorptive chromatography or column chromatography over silica gel or alumina, crystallization, preparatory thin layer chromatography, ion exchange chromatography, or HPLC.

The extent of modification can be assessed by measuring the absorbance of the nitropyridine thione, dinitropyridine dithione, pyridine thione, carboxylamidopyridine dithione and dicarboxyl-amidopyridine dithione group released via UV spectra. For the conjugation without a chromophore group, the modification or conjugation reaction can be monitored by LC-MS, preferably by HPLC-MS/MS, UPLC-QTOF mass spectrometry, or Capilary electrophoresis-mass spectrometry (CE-MS). The side chain cross-linkers described herein have diverse functional groups that can react with any cell-binding molecules, particularly a modified cell-binding molecule that possess a suitable substituent. For examples, the modified cell-binding molecules bearing an amino or hydroxyl substituent can react with drugs bearing an N-hydroxysuccinimide (NHS) ester, the modified cell-binding molecules bearing a thiol substituent can react with drugs bearing a maleimido or haloacetyl group. Additionally, the modified cell-binding molecules bearing a carbonyl (ketone or aldehyde) substituent either through protein engineering, enzymatical reaction or chemical modification can react with drugs bearing a hydrazide or an alkoxyamine. One skilled in the art can readily determine which modified drug-linker to be used based on the known reactivity of the available functional group on the modified cell-binding molecules.

Cell-Binding Agents

The cell-binding molecule, T or Cb or mAb, that comprises the conjugates and the modified cell-binding agents of the present invention may be of any kind presently known, or that become known, molecule that binds to, complexes with, or reacts with a moiety of a cell population sought to be therapeutically or otherwise biologically modified.

The cell binding molecules/agents include, but are not limited to, large molecular weight proteins such as, for example, antibody, an antibody-like protein, full-length antibodies (polyclonal antibodies, monoclonal antibodies, dimers, multimers, multispecific antibodies (e.g., bispecific antibodies); single chain antibodies; fragments of antibodies such as Fab, Fab′, F(ab′)₂, Fᵥ, [Parham, J. Immunol. 131, 2895-902 (1983)], fragments produced by a Fab expression library, anti-idiotypic (anti-Id) antibodies, CDR's, diabody, triabody, tetrabody, miniantibody, small immune proteins (SIP), and epitope-binding fragments of any of the above which immuno-specifically bind to cancer cell antigens, viral antigens, microbial antigens or a protein generated by the immune system that is capable of recognizing, binding to a specific antigen or exhibiting the desired biological activity (Miller et al (2003) J. of Immunology 170: 4854-61); interferons (such as type I, II, III); peptides; lymphokines such as IL-2, IL-3, IL-4, IL-5, IL-6, IL-6R, IL-10, IL-11, IL-16, IL-17, GM-CSF, interferon-gamma (IFN-7); hormones such as insulin, TRH (thyrotropin releasing hormones), MSH (melanocyte-stimulating hormone), steroid hormones, such as androgens and estrogens, melanocyte-stimulating hormone (MSH); growth factors and colony-stimulating factors such as epidermal growth factors (EGF), granulocyte-macrophage colony-stimulating factor (GM-CSF), transforming growth factors (TGF), such as TGFα, TGFβ, insulin and insulin like growth factors (IGF-I, IGF-II) G-CSF, M-CSF and GM-CSF [Burgess, Immunology Today, 5, 155-8 (1984)]; vaccinia growth factors (VGF); fibroblast growth factors (FGFs); smaller molecular weight proteins, poly-peptide, peptides and peptide hormones, such as bombesin, gastrin, gastrin-releasing peptide; platelet-derived growth factors; interleukin and cytokines, such as interleukin-2 (IL-2), interleukin-6 (IL-6), leukemia inhibitory factors, granulocyte-macrophage colony-stimulating factor (GM-CSF); vitamins, such as folate; apoproteins and glycoproteins, such as transferrin [O'Keefe et al, 260 J. Biol. Chem. 932-7 (1985)]; sugar-binding proteins or lipoproteins, such as lectins; cell nutrient-transport molecules; and small molecular inhibitors, such as prostate-specific membrane antigen (PSMA) inhibitors and small molecular tyrosine kinase inhibitors (TKI), non-peptides or any other cell binding molecule or substance, such as bioactive polymers (Dhar, et al, Proc. Natl. Acad. Sci. 2008, 105, 17356-61); fusion proteins; kinase inhibitors; gene-targeting agents; bioactive dendrimers (Lee, et al, Nat. Biotechnol. 2005, 23, 1517-26; Almutairi, et al; Proc. Natl. Acad. Sci. 2009, 106, 685-90); nanoparticles (Liong, et al, ACS Nano, 2008, 2, 1309-12; Medarova, et al, Nat. Med. 2007, 13, 372-7; Javier, et al, Bioconjugate Chem. 2008, 19, 1309-12); liposomes (Medinai, et al, Curr. Phar. Des. 2004, 10, 2981-9); viral capsides (Flenniken, et al, Viruses Nanotechnol. 2009, 327, 71-93).

In general, a monoclonal antibody is preferred as a cell-surface binding agent if an appropriate one is available. And the antibody may be murine, human, humanized, chimeric, or derived from other species.

Production of antibodies used in the present invention involves in vivo or in vitro procedures or combinations thereof. Methods for producing polyclonal anti-receptor peptide antibodies are well-known in the art, such as in U.S. Pat. No. 4,493,795 (to Nestor et al). A monoclonal antibody is typically made by fusing myeloma cells with the spleen cells from a mouse that has been immunized with the desired antigen (Kohler, G.; Milstein, C. (1975). Nature 256: 495-7). The detailed procedures are described in “Antibodies—A Laboratory Manual”, Harlow and Lane, eds., Cold Spring Harbor Laboratory Press, New York (1988), which is incorporated herein by reference. Particularly monoclonal antibodies are produced by immunizing mice, rats, hamsters or any other mammal with the antigen of interest such as the intact target cell, antigens isolated from the target cell, whole virus, attenuated whole virus, and viral proteins. Splenocytes are typically fused with myeloma cells using polyethylene glycol (PEG) 6000. Fused hybrids are selected by their sensitivity to HAT (hypoxanthine-aminopterin-thymine). Hybridomas producing a monoclonal antibody useful in practicing this invention are identified by their ability to immunoreact specified receptors or inhibit receptor activity on target cells.

A monoclonal antibody used in the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques, such as using protein-A affinity chromatography; anion, cation, hydrophobic, or size exclusive chromatographies (particularly by affinity for the specific antigen after protein A, and sizing column chromatography); centrifugation, differential solubility, or by any other standard technique for the purification of proteins.

Media useful for the preparation of these compositions are both well-known in the art and commercially available and include synthetic culture media. An exemplary synthetic medium is Dulbecco's minimal essential medium (DMEM; Dulbecco et al., Virol. 8, 396 (1959)) supplemented with 4.5 gm/l glucose, 0˜20 mM glutamine, 0˜20% fetal calf serum, several ppm amount of heavy metals, such as Cu, Mn, Fe, or Zn, etc, or/and the other heavy metals added in their salt forms, and with an anti-foaming agent, such as polyoxyethylene-polyoxypropylene block copolymer.

In addition, antibody-producing cell lines can also be created by techniques other than fusion, such as direct transformation of B lymphocytes with oncogenic DNA, or transfection with an oncovirus, such as Epstein-Barr virus (EBV, also called human herpesvirus 4 (HHV-4)) or Kaposi's sarcoma-associated herpesvirus (KSHV). See, U.S. Pat. Nos. 4,341,761; 4,399,121; 4,427,783; 4,444,887; 4,451,570; 4,466,917; 4,472,500; 4,491,632; 4,493,890. A monoclonal antibody may also be produced via an anti-receptor peptide or peptides containing the carboxyl terminal as described well-known in the art. See Niman et al., Proc. Natl. Acad. Sci. USA, 80: 4949-53 (1983); Geysen et al., Proc. Natl. Acad. Sci. USA, 82: 178-82 (1985); Lei et al. Biochemistry 34(20): 6675-88, (1995). Typically, the anti-receptor peptide or a peptide analog is used either alone or conjugated to an immunogenic carrier, as the immunogen for producing anti-receptor peptide monoclonal antibodies.

There are also a number of other well-known techniques for making monoclonal antibodies as binding molecules in this invention. Particularly useful are methods of making fully human antibodies. One method is phage display technology which can be used to select a range of human antibodies binding specifically to the antigen using methods of affinity enrichment. Phage display has been thoroughly described in the literature and the construction and screening of phage display libraries are well known in the art, see, e.g., Dente et al, Gene. 148(1):7-13 (1994); Little et al, Biotechnol Adv. 12(3): 539-55 (1994); Clackson et al., Nature 352: 264-8 (1991); Huse et al., Science 246: 1275-81 (1989).

Monoclonal antibodies derived by hybridoma technique from another species than human, such as mouse, can be humanized to avoid human anti-mouse antibodies when infused into humans. Among the more common methods of humanization of antibodies are complementarity-determining region grafting and resurfacing. These methods have been extensively described, see e.g. U.S. Pat. Nos. 5,859,205 and 6,797,492; Liu et al, Immunol Rev. 222: 9-27 (2008); Almagro et al, Front Biosci. 13: 1619-33 (2008); Lazar et al, Mol Immunol. 44(8): 1986-98 (2007); Li et al, Proc. Natl. Acad. Sci. USA. 103(10): 3557-62 (2006) each incorporated herein by reference. Fully human antibodies can also be prepared by immunizing transgenic mice, rabbits, monkeys, or other mammals, carrying large portions of the human immunoglobulin heavy and light chains, with an immunogen. Examples of such mice are: the Xenomouse. (Abgenix/Amgen), the HuMAb-Mouse (Medarex/BMS), the VelociMouse (Regeneron), see also U.S. Pat. Nos. 6,596,541, 6,207,418, 6,150,584, 6,111,166, 6,075,181, 5,922,545, 5,661,016, 5,545,806, 5,436,149 and 5,569,825. In human therapy, murine variable regions and human constant regions can also be fused to construct called “chimeric antibodies” that are considerably less immunogenic in man than murine mAbs (Kipriyanov et al, Mol Biotechnol. 26: 39-60 (2004); Houdebine, Curr Opin Biotechnol. 13: 625-9 (2002) each incorporated herein by reference). In addition, site-directed mutagenesis in the variable region of an antibody can result in an antibody with higher affinity and specificity for its antigen (Brannigan et al, Nat Rev Mol Cell Biol. 3: 964-70, (2002)); Adams et al, J Immunol Methods. 231: 249-60 (1999)) and exchanging constant regions of a mAb can improve its ability to mediate effector functions of binding and cytotoxicity.

Antibodies immunospecific for a malignant cell antigen can also be obtained commercially or produced by any method known to one of skill in the art such as, e.g., chemical synthesis or recombinant expression techniques. The nucleotide sequence encoding antibodies immune-specific for a malignant cell antigen can be obtained commercially, e.g., from the GenBank database or a database like it, the literature publications, or by routine cloning and sequencing.

Apart from an antibody, a peptide or protein that bind/block/target or in some other way interact with the epitopes or corresponding receptors on a targeted cell can be used as a binding molecule. These peptides or proteins could be any random peptide or proteins that have an affinity for the epitopes or corresponding receptors and they don't necessarily have to be of the immune-globulin family. These peptides can be isolated by similar techniques as for phage display antibodies (Szardenings, J Recept Signal Transduct Res. 2003, 23(4): 307-49). The use of peptides from such random peptide libraries can be similar to antibodies and antibody fragments. The binding molecules of peptides or proteins may be conjugated on or linked to a large molecules or materials, such as, but is not limited, an albumin, a polymer, a liposome, a nano particle, a dendrimer, as long as such attachment permits the peptide or protein to retain its antigen binding specificity.

Examples of antibodies used for conjugation of drugs via the linkers of this prevention for treating cancer, autoimmune disease, and/or infectious disease include, but are not limited to, 3F8 (anti-GD2), Abagovomab (anti CA-125), Abciximab (anti CD41 (integrin alpha-IIb), Adalimumab (anti-TNF-α), Adecatumumab (anti-EpCAM, CD326), Afelimomab (anti-TNF-α); Afutuzumab (anti-CD20), Alacizumab pegol (anti-VEGFR2), ALD518 (anti-IL-6), Alemtuzumab (Campath, MabCampath, anti-CD52), Altumomab (anti-CEA), Anatumomab (anti-TAG-72), Anrukinzumab (IMA-638, anti-IL-13), Apolizumab (anti-HLA-DR), Arcitumomab (anti-CEA), Aselizumab (anti-L-selectin (CD62L), Atlizumab (tocilizumab, Actemra, RoActemra, anti-IL-6 receptor), Atorolimumab (anti-Rhesus factor), Bapineuzumab (anti-beta amyloid), Basiliximab (Simulect, antiCD25 (a chain of IL-2 receptor), Bavituximab (anti-phosphatidylserine), Bectumomab (LymphoScan, anti-CD22), Belimumab (Benlysta, LymphoStat-B, anti-BAFF), Benralizumab (anti-CD125), Bertilimumab (anti-CCL11 (eotaxin-1)), Besilesomab (Scintimun, anti-CEA-related antigen), Bevacizumab (Avastin, anti-VEGF-A), Biciromab (FibriScint, anti-fibrin II beta chain), Bivatuzumab (anti-CD44 v6), Blinatumomab (BiTE, anti-CD19), Brentuximab (cAC10, anti-CD30 TNFRSF8), Briakinumab (anti-IL-12, IL-23) Canakinumab (Ilaris, anti-IL-1), Cantuzumab (C242, anti-CanAg), Capromab, Catumaxomab (Removab, anti-EpCAM, anti-CD3), CC49 (anti-TAG-72), Cedelizumab (anti-CD4), Certolizumab pegol (Cimzia anti-TNF-α), Cetuximab (Erbitux, IMC-C225, anti-EGFR), Citatuzumab bogatox (anti-EpCAM), Cixutumumab (anti-IGF-1), Clenoliximab (anti-CD4), Clivatuzumab (anti-MUC1), Conatumumab (anti-TRAIL-R2), CR6261 (anti-Influenza A hemagglutinin), Dacetuzumab (anti-CD40), Daclizumab (Zenapax, anti-CD25 (a chain of IL-2 receptor)), Daratumumab (anti-CD38 (cyclic ADP ribose hydrolase), Denosumab (Prolia, anti-RANKL), Detumomab (anti-B-lymphoma cell), Dorlimomab, Dorlixizumab, Ecromeximab (anti-GD3 ganglioside), Eculizumab (Soliris, anti-C5), Edobacomab (anti-endotoxin), Edrecolomab (Panorex, MAb17-1A, anti-EpCAM), Efalizumab (Raptiva, anti-LFA-1 (CD11a), Efungumab (Mycograb, anti-Hsp90), Elotuzumab (anti-SLAMF7), Elsilimomab (anti-IL-6), Enlimomab pegol (anti-ICAM-1 (CD54)), Epitumomab (anti-episialin), Epratuzumab (anti-CD22), Erlizumab (anti-ITGB2 (CD18)), Ertumaxomab (Rexomun, anti-HER2/neu, CD3), Etaracizumab (Abegrin, anti-integrin αᵥβ₃), Exbivirumab (anti-hepatitis B surface antigen), Fanolesomab (NeutroSpec, anti-CD15), Faralimomab (anti-interferon receptor), Farletuzumab (anti-folate receptor 1), Felvizumab (anti-respiratory syncytial virus), Fezakinumab (anti-IL-22), Figitumumab (anti-IGF-1 receptor), Fontolizumab (anti-IFN-7), Foravirumab (anti-rabies virus glycoprotein), Fresolimumab (anti-TGF-β), Galiximab (anti-CD80), Gantenerumab (anti-beta amyloid), Gavilimomab (anti-CD147 (basigin)), Gemtuzumab (anti-CD33), Girentuximab (anti-carbonic anhydrase 9), Glembatumumab (CR011, anti-GPNMB), Golimumab (Simponi, anti-TNF-α), Gomiliximab (anti-CD23 (IgE receptor)), Ibalizumab (anti-CD4), Ibritumomab (anti-CD20), Igovomab (Indimacis-125, anti-CA-125), Imciromab (Myoscint, anti-cardiac myosin), Infliximab (Remicade, anti-TNF-α), Intetumumab (anti-CD51), Inolimomab (anti-CD25 (a chain of IL-2 receptor)), Inotuzumab (anti-CD22), Ipilimumab (anti-CD152), Iratumumab (anti-CD30 (TNFRSF8)), Keliximab (anti-CD4), Labetuzumab (CEA-Cide, anti-CEA), Lebrikizumab (anti-IL-13), Lemalesomab (anti-NCA-90 (granulocyte antigen)), Lerdelimumab (anti-TGF beta 2), Lexatumumab (anti-TRAIL-R2), Libivirumab (anti-hepatitis B surface antigen), Lintuzumab (anti-CD33), Lucatumumab (anti-CD40), Lumiliximab (anti-CD23 (IgE receptor), Mapatumumab (anti-TRAIL-R1), Maslimomab (anti-T-cell receptor), Matuzumab (anti-EGFR), Mepolizumab (Bosatria, anti-IL-5), Metelimumab (anti-TGF beta 1), Milatuzumab (anti-CD74), Minretumomab (anti-TAG-72), Mitumomab (BEC-2, anti-GD3 ganglioside), Morolimumab (anti-Rhesus factor), Motavizumab (Numax, anti-respiratory syncytial virus), Muromonab-CD3 (Orthoclone OKT3, anti-CD3), Nacolomab (anti-C242), Naptumomab (anti-5T4), Natalizumab (Tysabri, anti-integrin α₄), Nebacumab (anti-endotoxin), Necitumumab (anti-EGFR), Nerelimomab (anti-TNF-α), Nimotuzumab (Theracim, Theraloc, anti-EGFR), Nofetumomab, Ocrelizumab (anti-CD20), Odulimomab (Afolimomab, anti-LFA-1 (CD11a)), Ofatumumab (Arzerra, anti-CD20), Olaratumab (anti-PDGF-R α), Omalizumab (Xolair, anti-IgE Fc region), Oportuzumab (anti-EpCAM), Oregovomab (OvaRex, anti-CA-125), Otelixizumab (anti-CD3), Pagibaximab (anti-lipoteichoic acid), Palivizumab (Synagis, Abbosynagis, anti-respiratory syncytial virus), Panitumumab (Vectibix, ABX-EGF, anti-EGFR), Panobacumab (anti-Pseudomonas aeruginosa), Pascolizumab (anti-IL-4), Pemtumomab (Theragyn, anti-MUC1), Pertuzumab (Omnitarg, 2C4, anti-HER2/neu), Pexelizumab (anti-C5), Pintumomab (anti-adenocarcinoma antigen), Priliximab (anti-CD4), Pritumumab (anti-vimentin), PRO 140 (anti-CCR5), Racotumomab (1E10, anti-(N-glycolylneuraminic acid (NeuGc, NGNA)-gangliosides GM3)), Rafivirumab (anti-rabies virus glycoprotein), Ramucirumab (anti-VEGFR2), Ranibizumab (Lucentis, anti-VEGF-A), Raxibacumab (anti-anthrax toxin, protective antigen), Regavirumab (anti-cytomegalovirus glycoprotein B), Reslizumab (anti-IL-5), Rilotumumab (anti-HGF), Rituximab (MabThera, Rituxanmab, anti-CD20), Robatumumab (anti-IGF-1 receptor), Rontalizumab (anti-IFN-α), Rovelizumab (LeukArrest, anti-CD11, CD18), Ruplizumab (Antova, anti-CD154 (CD40L)), Satumomab (anti-TAG-72), Sevirumab (anti-cytomegalovirus), Sibrotuzumab (anti-FAP), Sifalimumab (anti-IFN-α), Siltuximab (anti-IL-6), Siplizumab (anti-CD2), (Smart) MI95 (anti-CD33), Solanezumab (anti-beta amyloid), Sonepcizumab (anti-sphingosine-1-phosphate), Sontuzumab (anti-episialin), Stamulumab (anti-myostatin), Sulesomab (LeukoScan, (anti-NCA-90 (granulocyte antigen), Tacatuzumab (anti-alpha-fetoprotein), Tadocizumab (anti-integrin α_{IIb}β₃), Talizumab (anti-IgE), Tanezumab (anti-NGF), Taplitumomab (anti-CD19), Tefibazumab (Aurexis, (anti-clumping factor A), Telimomab, Tenatumomab (anti-tenascin C), Teneliximab (anti-CD40), Teplizumab (anti-CD3), TGN1412 (anti-CD28), Ticilimumab (Tremelimumab, (anti-CTLA-4), Tigatuzumab (anti-TRAIL-R2), TNX-650 (anti-IL-13), Tocilizumab (Atlizumab, Actemra, RoActemra, (anti-IL-6 receptor), Toralizumab (anti-CD154 (CD40L)), Tositumomab (anti-CD20), Trastuzumab (Herceptin, (anti-HER2/neu), Tremelimumab (anti-CTLA-4), Tucotuzumab celmoleukin (anti-EpCAM), Tuvirumab (anti-hepatitis B virus), Urtoxazumab (anti-Escherichia coli), Ustekinumab (Stelara, anti-IL-12, IL-23), Vapaliximab (anti-AOC3 (VAP-1)), Vedolizumab, (anti-integrin α₄β₇), Veltuzumab (anti-CD20), Vepalimomab (anti-AOC3 (VAP-1), Visilizumab (Nuvion, anti-CD3), Vitaxin (anti-vascular integrin avb3), Volociximab (anti-integrin α₅β₁), Votumumab (HumaSPECT, anti-tumor antigen CTAA16.88), Zalutumumab (HuMax-EGFr, (anti-EGFR), Zanolimumab (HuMax-CD4, anti-CD4), Ziralimumab (anti-CD147 (basigin)), Zolimomab (anti-CD5), Etanercept (Enbrel®), Alefacept (Amevive®), Abatacept (Orencia®), Rilonacept (Arcalyst), 14F7 [anti-IRP-2 (Iron Regulatory Protein 2)], 14G2a (anti-GD2 ganglioside, from Nat. Cancer Inst. for melanoma and solid tumors), J591 (anti-PSMA, Weill Cornell Medical School for prostate cancers), 225.285 [anti-HMW-MAA (High molecular weight-melanoma-associated antigen), Sorin Radiofarmaci S.R.L. (Milan, Italy) for melanoma], COL-1 (anti-CEACAM3, CGM1, from Nat. Cancer Inst. USA for colorectal and gastric cancers), CYT-356 (Oncoltad®, for prostate cancers), HNK20 (OraVax Inc. for respiratory syncytial virus), ImmuRAIT (from Immunomedics for NHL), Lym-1 (anti-HLA-DR10, Peregrine Pharm. for Cancers), MAK-195F [anti-TNF (tumor necrosis factor; TNFA, TNF-alpha; TNFSF2), from Abbott/Knoll for Sepsis toxic shock], MEDI-500 [T10B9, anti-CD3, TRαβ (T cell receptor alpha/beta), complex, from MedImmune Inc for Graft-versus-host disease], RING SCAN [anti-TAG 72 (tumour associated glycoprotein 72), from Neoprobe Corp. for Breast, Colon and Rectal cancers], Avicidin (anti-EPCAM (epithelial cell adhesion molecule), anti-TACSTD1 (Tumor-associated calcium signal transducer 1), anti-GA733-2 (gastrointestinal tumor-associated protein 2), anti-EGP-2 (epithelial glycoprotein 2); anti-KSA; KS1/4 antigen; M4S; tumor antigen 17-1A; CD326, from NeoRx Corp. for Colon, Ovarian, Prostate cancers and NHL]; LymphoCide (Immunomedics, NJ), Smart ID10 (Protein Design Labs), Oncolym (Techniclone Inc, CA), Allomune (BioTransplant, CA), anti-VEGF (Genentech, CA); CEAcide (Immunomedics, NJ), IMC-1C11 (ImClone, NJ) and Cetuximab (ImClone, NJ).

Other antibodies as cell binding molecules/ligands include, but are not limited to, are antibodies against the following antigens: Aminopeptidase N (CD13), Annexin A1, B7-H3 (CD276, various cancers), CA125 (ovarian), CA15-3 (carcinomas), CA19-9 (carcinomas), L6 (carcinomas), Lewis Y (carcinomas), Lewis X (carcinomas), alpha fetoprotein (carcinomas), CA242 (colorectal), placental alkaline phosphatase (carcinomas), prostate specific antigen (prostate), prostatic acid phosphatase (prostate), epidermal growth factor (carcinomas), CD2 (Hodgkin's disease, NHL lymphoma, multiple myeloma), CD3 epsilon (T cell lymphoma, lung, breast, gastric, ovarian cancers, autoimmune diseases, malignant ascites), CD19 (B cell malignancies), CD20 (non-Hodgkin's lymphoma), CD22 (leukemia, lymphoma, multiple myeloma, SLE), CD30 (Hodgkin's lymphoma), CD33 (leukemia, autoimmune diseases), CD38 (multiple myeloma), CD40 (lymphoma, multiple myeloma, leukemia (CLL)), CD51 (Metastatic melanoma, sarcoma), CD52 (leukemia), CD56 (small cell lung cancers, ovarian cancer, Merkel cell carcinoma, and the liquid tumor, multiple myeloma), CD66e (cancers), CD70 (metastatic renal cell carcinoma and non-Hodgkin lymphoma), CD74 (multiple myeloma), CD80 (lymphoma), CD98 (cancers), mucin (carcinomas), CD221 (solid tumors), CD227 (breast, ovarian cancers), CD262 (NSCLC and other cancers), CD309 (ovarian cancers), CD326 (solid tumors), CEACAM3 (colorectal, gastric cancers), CEACAM5 (carcinoembryonic antigen; CEA, CD66e) (breast, colorectal and lung cancers), DLL3 (delta-like-3), DLL4 (delta-like-4), EGFR (Epidermal Growth Factor Receptor, various cancers), CTLA4 (melanoma), CXCR4 (CD184, Heme-oncology, solid tumors), Endoglin (CD105, solid tumors), EPCAM (epithelial cell adhesion molecule, bladder, head, neck, colon, NHL prostate, and ovarian cancers), ERBB2 (Epidermal Growth Factor Receptor 2; lung, breast, prostate cancers), FCGR1 (autoimmune diseases), FOLR (folate receptor, ovarian cancers), GD2 ganglioside (cancers), G-28 (a cell surface antigen glyvolipid, melanoma), GD3 idiotype (cancers), Heat shock proteins (cancers), HER1 (lung, stomach cancers), HER2 (breast, lung and ovarian cancers), HLA-DR10 (NHL), HLA-DRB (NHL, B cell leukemia), human chorionic gonadotropin (carcinoma), IGF1R (insulin-like growth factor 1 receptor, solid tumors, blood cancers), IL-2 receptor (interleukin 2 receptor, T-cell leukemia and lymphomas), IL-6R (interleukin 6 receptor, multiple myeloma, RA, Castleman's disease, IL6 dependent tumors), Integrins (αvβ3, α5β1, α6β4, αllβ3, α5β5, αvβ5, for various cancers), MAGE-1 (carcinomas), MAGE-2 (carcinomas), MAGE-3 (carcinomas), MAGE 4 (carcinomas), anti-transferrin receptor (carcinomas), p97 (melanoma), MS4A1 (membrane-spanning 4-domains subfamily A member 1, Non-Hodgkin's B cell lymphoma, leukemia), MUC1 or MUC1-KLH (breast, ovarian, cervix, bronchus and gastrointestinal cancer), MUC16 (CA125) (Ovarian cancers), CEA (colorectal), gp100 (melanoma), MART1 (melanoma), MPG (melanoma), MS4A1 (membrane-spanning 4-domains subfamily A, small cell lung cancers, NHL), Nucleolin, Neu oncogene product (carcinomas), P21 (carcinomas), Paratope of anti-(N-glycolylneuraminic acid, Breast, Melanoma cancers), PLAP-like testicular alkaline phosphatase (ovarian, testicular cancers), PSMA (prostate tumors), PSA (prostate), ROBO4, TAG 72 (tumour associated glycoprotein 72, AML, gastric, colorectal, ovarian cancers), T cell transmembrane protein (cancers), Tie (CD202b), TNFRSF10B (tumor necrosis factor receptor superfamily member 10B, cancers), TNFRSF13B (tumor necrosis factor receptor superfamily member 13B, multiple myeloma, NHL, other cancers, RA and SLE), TPBG (trophoblast glycoprotein, Renal cell carcinoma), TRAIL-R1 (Tumor necrosis apoprosis Inducing ligand Receptor 1, lymphoma, NHL, colorectal, lung cancers), VCAM-1 (CD106, Melanoma), VEGF, VEGF-A, VEGF-2 (CD309) (various cancers). Some other tumor associated antigens recognized by antibodies have been reviewed (Gerber, et al, mAbs 1:3, 247-53 (2009); Novellino et al, Cancer Immunol Immunother. 54(3), 187-207 (2005). Franke, et al, Cancer Biother Radiopharm. 2000, 15, 459-76).

The cell-binding agents, more preferred antibodies, can be any agents that are able to against tumor cells, virus infected cells, microorganism infected cells, parasite infected cells, autoimmune cells, activated cells, myeloid cells, activated T-cells, B cells, or melanocytes. More specifically the cell binding agents can be any agent/molecule that is able to against any one of the following antigens or receptors: CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD12w, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202(a, b), CD203, CD203c, CD204, CD205, CD206, CD207, CD208, CD209, CD210, CDw210a, CDw210b, CD211, CD212, CD213, CD213a1, CD213a2, CD214, CD215, CD216, CD217, CD218, CD218a, CD218, CD219b, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CR, CR1, CRGF, CRIPTO, CXCR5, LY64, TDGF1, 4-1BB, APO2, ASLG659, BMPR1B, 4-1BB, 5AC, 5T4 (Trophoblast glycoprotein, TPBG, 5T4, Wnt-Activated Inhibitory Factor 1 or WAIF1), Adenocarcinomaantigen, AGS-5, AGS-22M6, Activin receptor-like kinase 1, AFP, AKAP-4, ALK, Alpha intergrin, Alpha v beta6, Amino-peptidase N, Amyloid beta, Androgen receptor, Angiopoietin 2, Angiopoietin 3, Annexin A1, Anthrax toxin-protective antigen, Anti-transferrin receptor, AOC3 (VAP-1), B7-H3, Bacillus anthracisanthrax, BAFF (B-cell activating factor), B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11(C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD115), CSF2 (colony stimulating factor 2, Granulocyte-macrophage colony-stimulating factor (GM-CSF)), CTLA4 (cytotoxic T-lymphocyte associated protein 4), CTAA16.88 tumor antigen, CXCR4 (CD184), C-X-C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxintype-1, E. coli shiga toxintype-2, ED-B, EGFL7 (EGF-like domain-containing protein 7), EGFR, EGFRII, EGFRvIII, Endoglin (CD105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation proteinalpha), FCGR1, alpha-Fetoprotein, Fibrin II, beta chain, Fibronectin extra domain-B, FOLR (folate receptor), Folate receptor alpha, Folate hydrolase, Fos-related antigen 1, F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influeza hemagglutinin, IgE, Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-16, IL-17, IL-17A, IL-20, IL-22, IL-23, IL31RA, ILGF2 (Insulin-like growth factor 2), Integrins (α4, α_{IIb}β₃, αvβ3, α₄β₇, α5β1, α6β4, α7β7, αllβ3, α5β5, αvβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (meso-thelin), MUC1 (Mucin 1, cell surfaceassociated (MUC1) orpolymorphic epithelial mucin (PEM)), MUC1-KLH, MUC16 (CA125), MCP1(monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1, CD279), PDGF-Rα (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R₁ (Tumor necrosis apoprosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TROP-2, TRP-2, Tyrosinase, VCAM-1 (CD106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

In another specific embodiment, the cell-binding molecule can be a ligand or a receptor agonist selected from: folate derivatives (binding to the folate receptor, a protein over-expressed in ovarian cancer and in other malignancies) (Low, P. S. et al 2008, Acc. Chem. Res. 41, 120-9); glutamic acid urea derivatives (binding to the prostate specific membrane antigen, a surface marker of prostate cancer cells) (Hillier, S. M. et al, 2009, Cancer Res. 69, 6932-40); Somatostatin (also known as growth hormone-inhibiting hormone (GHIH) or somatotropin release-inhibiting factor (SRIF)) or somatotropin release-inhibiting hormone) and its analogues such as octreotide (Sandostatin) and lanreotide (Somatuline) (particularly for neuroendocrine tumors, GH-producing pituitary adenoma, paraganglioma, nonfunctioning pituitary adenoma, pheochromocytomas) (Ginj, M., et al, 2006, Proc. Natl. Acad. Sci. U.S.A. 103, 16436-41); Somatostatin receptor subtypes (sst1, sst2, sst3, sst4, and sst5) in GH-secreting pituitaryadenomas (Reubi J. C., Landolt, A. M. 1984 J. Clin. Endocrinol Metab 59: 1148-51; Reubi J. C., Landolt A. M. 1987 J Clin Endocrinol Metab 65: 65-73; Moyse E, et al, J Clin Endocrinol Metab 61: 98-103), gastroenteropancreatic tumors (Reubi J. C., et al, 1987 J Clin Endocrinol Metab 65: 1127-34; Reubi, J. C, et al, 1990 Cancer Res 50: 5969-77), pheochromocytomas (Epel-baum J, et al 1995 J Clin Endocrinol Metab 80:1837-44; Reubi J. C., et al, 1992 J Clin Endocrinol Metab 74: 1082-9), neuroblastomas (Prevost G, 1996 Neuroendocrinology 63:188-197; Moertel, C. L, et al 1994 Am J Clin Path 102:752-756), medullary thyroid cancers (Reubi, J. C, et al 1991 Lab Invest 64:567-573) small cell lung cancers (Sagman U, et al, 1990 Cancer 66:2129-2133), meningiomas, medulloblastomas, or gliomas (Reubi J. C., et al 1986 J Clin Endocrinol Metab 63: 433-8; Reubi J. C., et al 1987 Cancer Res 47: 5758-64; Fruhwald, M. C, et al 1999 Pediatr Res 45: 697-708), breast carcinomas (Reubi J. C., et al 1990 Int J Cancer 46: 416-20; Srkalovic G, et al 1990 J Clin Endocrinol Metab 70: 661-669), lymphomas (Reubi J. C., et al 1992, Int J Cancer 50: 895-900), renal cell cancers (Reubi J. C., et al 1992, Cancer Res 52: 6074-6078), mesenchymal tumors (Reubi J. C., et al 1996 Cancer Res 56: 1922-31), prostatic (Reubi J. C., et al 1995, J. Clin. Endocrinol Metab 80: 2806-14; et al 1989, Prostate 14:191-208; Halmos G, et al J. Clin. Endocrinol Metab 85: 2564-71), ovarian (Halmos, G, et al, 2000 J Clin Endocrinol Metab 85: 3509-12; Reubi J. C., et al 1991 Am J Pathol 138:1267-72), gastric (Reubi J. C., et al 1999, Int J Cancer 81: 376-86; Miller, G. V, 1992 Br J Cancer 66: 391-95), hepatocellular (Kouroumalis E, et al 1998 Gut 42: 442-7; Reubi J. C., et al 1999 Gut 45: 66-774) and nasopharyngeal carcinomas (Loh K. S, et al, 2002 Virchows Arch 441: 444-8); Aromatic sulfonamides (specific to carbonic anhydrase IX) (a marker of hypoxia and of renal cell carcinoma) (Neri, D., et al, Nat. Rev. Drug Discov. 2011, 10, 767-7); Pituitary adenylate cyclase activating peptides (PACAP) (PAC1) for pheochromocytomas and paragangliomas; Vasoactive intestinal peptides (VIP) and their receptor subtypes (VPAC1, VPAC2); α-Melanocyte-stimulating hormone (α-MSH) receptors; Cholecystokinin (CCK)/gastrin receptors and their receptor subtypes (CCK1 (formerly CCK-A) and CCK2; Bombesin(Pyr-Gln-Arg-Leu-Gly-Asn-Gln-Trp-Ala-Val-Gly-His-Leu-Met-NH₂)/gastrin-releasing peptide (GRP) and their receptor subtypes (BB1, GRP receptor subtype (BB2), the BB3 and BB4) (Ohlsson, B., et al, 1999, Scand. J. Gastroenterology 34(12): 1224-9; Weber, H. C, 2009, Cur. Opin. Endocri. Diab. Obesity 16(1): 66-71, Gonzalez N, et al, 2008, Cur. Opin. Endocri. Diab. Obesity 15(1), 58-64); Neurotensin receptors and its receptor subtypes (NTR1, NTR2, NTR3); Substance P receptors and their receptor subtypes (such as NK1 receptor for Glial tumors, Hennig I. M., et al 1995 Int. J. Cancer 61, 786-792); Neuropeptide Y (NPY) receptors and its receptor subtypes (Y1-Y6); Homing Peptides include RGD (Arg-Gly-Asp), NGR (Asn-Gly-Arg), the dimeric and multimeric cyclic RGD peptides (e.g. cRGDfV) (Laakkonen P, Vuorinen K. 2010, Integr Biol (Camb). 2(7-8): 326-337; Chen K, Chen X. 2011, Theranostics. 1:189-200; Garanger E, et al, Anti-Cancer Agents Med Chem. 7 (5): 552-558; Kerr, J. S. et al, Anticancer Research, 19(2A), 959-968; Thumshirn, G, et al, 2003 Chem. Eur. J. 9, 2717-2725), and TAASGVRSMH or LTLRWVGLMS (chondroitin sulfate proteoglycan NG2 receptor) and F3 peptides (31 amino acid peptide that binds to cell surface-expressed nucleolin receptor) (Zitzmann, S., 2002 Cancer Res., 62, 18, pp. 5139-5143, Temminga, K., 2005, Drug Resistance Updates, 8, 381-402; P. Laakkonen and K. Vuorinen, 2010 Integrative Biol, 2(7-8), 326-337; M. A. Burg, 1999 Cancer Res., 59(12), 2869-2874; K. Porkka, et al 2002, Proc. Nat. Acad. Sci. USA 99(11), 7444-9); Cell Penetrating Peptides (CPPs) (Nakase I, et al, 2012, J. Control Release. 159(2), 181-188); Peptide Hormones, such as luteinizing hormone-releasing hormone (LHRH) agonists and antagonists, and gonadotropin-releasing hormone (GnRH) agonist, acts by targeting follicle stimulating hormone (FSH) and luteinising hormone (LH), as well as testosterone production, e.g. buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloro-Phe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂) (Thundimadathil, J., J. Amino Acids, 2012, 967347, doi:10.1155/2012/967347; Boccon-Gibod, L.; et al, 2011, Therapeutic Advances in Urology 3(3): 127-140; Debruyne, F., 2006, Future Oncology, 2(6), 677-696; Schally A. V; Nagy, A. 1999 Eur J Endocrinol 141:1-14; Koppan M, et al 1999 Prostate 38:151-158); and Pattern Recognition Receptors (PRRs), such as Toll-like receptors (TLRs), C-type lectins and Nodlike Receptors (NLRs) (Fukata, M., et al, 2009, Semin. Immunol. 21, 242-253; Maisonneuve, C., et al, 2014, Proc. Natl. Acad. Sci. U.S.A. 111, 1-6; Botos, I., et al, 2011, Structure 19, 447-459; Means, T. K., et al, 2000, Life Sci. 68, 241-258) that range in size from small molecules (imiquimod, guanosine and adenosine analogs) to large and complex biomacromolecules such as lipopolysaccharide (LPS), nucleic acids (CpG DNA, polyI:C) and lipopeptides (Pam3CSK4) (Kasturi, S. P., et al, 2011, Nature 470, 543-547; Lane, T., 2001, J. R. Soc. Med. 94, 316; Hotz, C., and Bourquin, C., 2012, Oncoimmunology 1, 227-228; Dudek, A. Z., et al, 2007, Clin. Cancer Res. 13, 7119-25); Calcitonin receptors which is a 32-amino-acid neuropeptide involved in the regulation of calcium levels largely through its effects on osteoclasts and on the kidney (Zaidi M, et al, 1990 Crit Rev Clin Lab Sci 28, 109-174; Gorn, A. H., et al 1995 J Clin Invest 95:2680-91); And integrin receptors and their receptor subtypes (such as α_{V}β₁, α_{V}β₃, α_{V}β₅, α_{V}β₆, α₆β₄, α₇β₁, α_{L}β₂, α_{IIb}β₃, etc.) which generally play important roles in angiogenesis are expressed on the surfaces of a variety of cells, in particular, of osteoclasts, endothelial cells and tumor cells (Ruoslahti, E. et al, 1994 Cell 77, 477-8; Albelda, S. M. et al, 1990 Cancer Res., 50, 6757-64). Short peptides, GRGDSPK and Cyclic RGD pentapeptides, such as cyclo(RGDfV) (L1) and its derives [cyclo(-N(Me)R-GDfV), cyclo(R-Sar-DfV), cyclo-(RG-N(Me)D-fV), cyclo(RGD-N(Me)f-V), cyclo(RGDf-N(Me)V-)(Cilengitide)] have shown high binding affinities of the intergrin receptors (Dechantsreiter, M. A. et al, 1999 J. Med. Chem. 42, 3033-40, Goodman, S. L., et al, 2002 J. Med. Chem. 45, 1045-51).

The cell-binding molecule/ligands or cell receptor agonists can be Ig-based and non-Ig-based protein scaffold molecules. The Ig-Based scaffolds can be selected, but not limited, from Nanobody (a derivative of VHH (camelid Ig)) (Muyldermans S., 2013 Annu Rev Biochem. 82, 775-97); Domain antibodies (dAb, a derivative of VH or VL domain) (Holt, L. J, et al, 2003, Trends Biotechnol. 21, 484-90); Bispecific T cell Engager (BiTE, a bispecific diabody) (Baeuerle, P. A, et al, 2009, Curr. Opin. Mol. Ther. 11, 22-30); Dual Affinity ReTargeting (DART, a bispecific diabody) (Moore P. A. P, et al. 2011, Blood 117(17), 4542-51); Tetravalent tandem antibodies (TandAb, a dimerized bispecific diabody) (Cochlovius, B, et al. 2000, Cancer Res. 60(16):4336-4341). The Non-Ig scaffolds can be selected, but not limited, from Anticalin (a derivative of Lipocalins) (Skerra A. 2008, FEBS J., 275(11): 2677-83; Beste G, et al, 1999 Proc. Nat. Acad. USA. 96(5):1898-903; Skerra, A. 2000 Biochim Biophys Acta, 1482(1-2): 337-50; Skerra, A. 2007, Curr Opin Biotechnol. 18(4): 295-304; Skerra, A. 2008, FEBS J. 275(11):2677-83); Adnectins (10th FN3 (Fibronectin)) (Koide, A, et al, 1998 J. Mol. Biol., 284(4):1141-51; Batori V, 2002, Protein Eng. 15(12): 1015-20; Tolcher, A. W, 2011, Clin. Cancer Res. 17(2): 363-71; Hackel, B. J, 2010, Protein Eng. Des. Sel. 23(4): 211-19); Designed Ankyrin Repeat Proteins (DARPins) (a derivative of ankrin repeat (AR) proteins) (Boersma, Y. L, et al, 2011 Curr Opin Biotechnol. 22(6): 849-57), e.g. DARPin C9, DARPin Ec4 and DARPin E69_LZ3_E01 (Winkler J, et al, 2009 Mol Cancer Ther. 8(9), 2674-83; Patricia M-K. M., et al, Clin Cancer Res. 2011; 17(1):100-10; Boersma Y. L, et al, 2011 J. Biol. Chem. 286(48), 41273-85); Avimers (a domain A/low-density lipoprotein (LDL) receptor) (Boersma Y. L, 2011 J. Biol. Chem. 286(48): 41273-41285; Silverman J, et al, 2005 Nat. Biotechnol., 23(12):1556-61).

Examples of the small molecule structures of the cell-binding molecules/ligands or cell receptor agonists of the patent application are the following: LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αᵥβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor,), LB23 (for an androgen receptor), LB24 (Cilengitide/cyclo(-RGDfV-) for an αᵥ intergrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a neucleoside analog), LB53 (Erlotinib analog) and LB54 (Lapatinib analog) which are shown in the following structures:

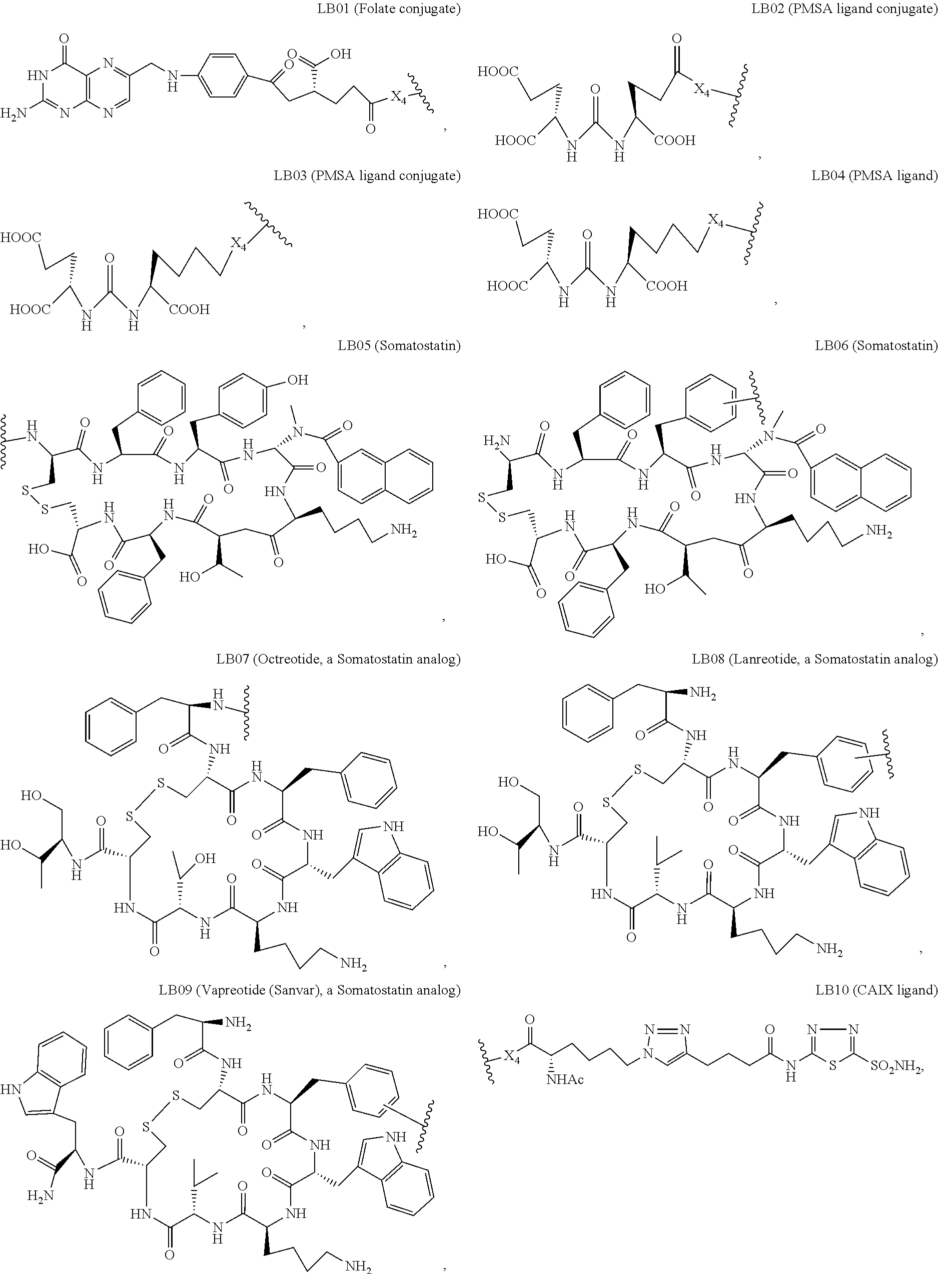

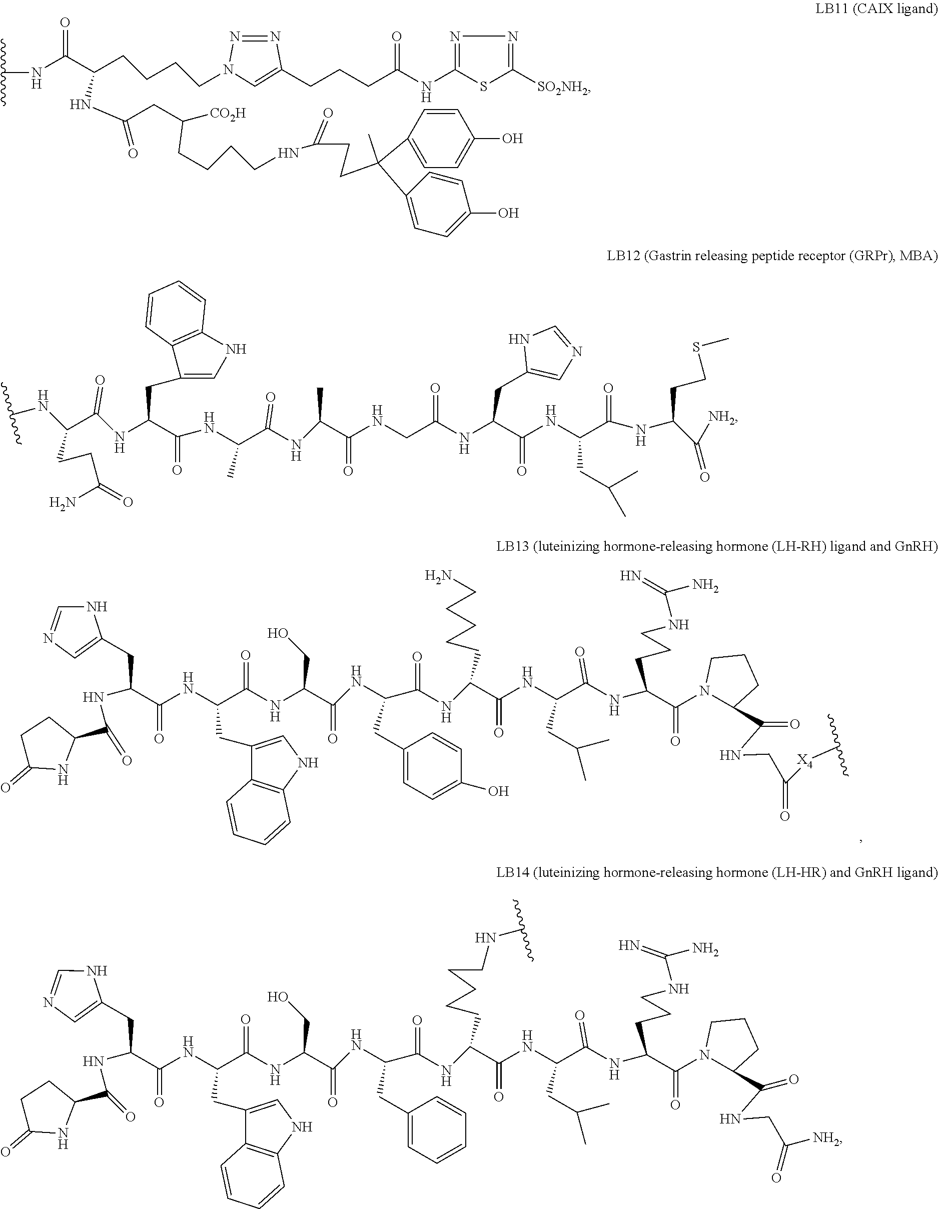

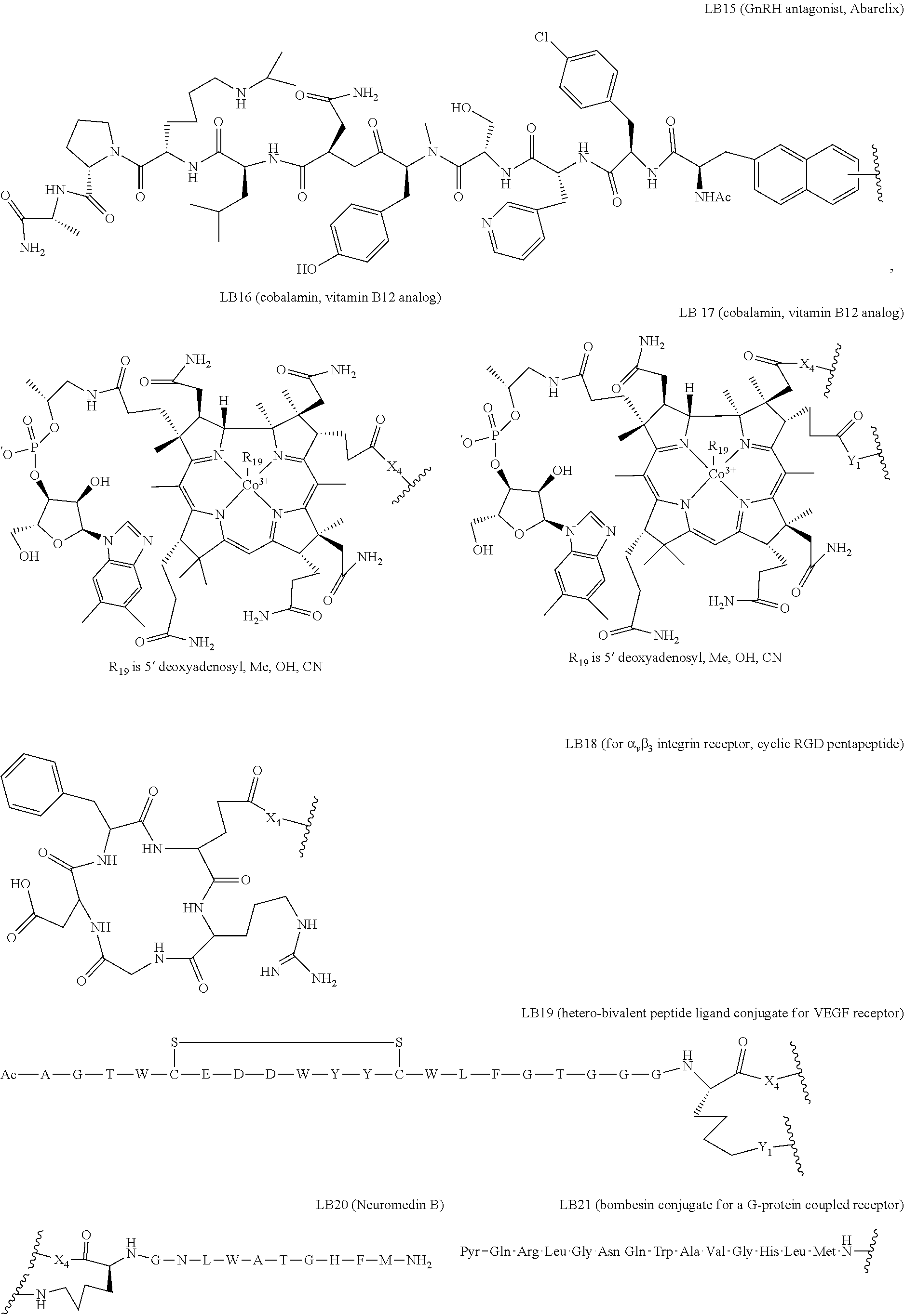

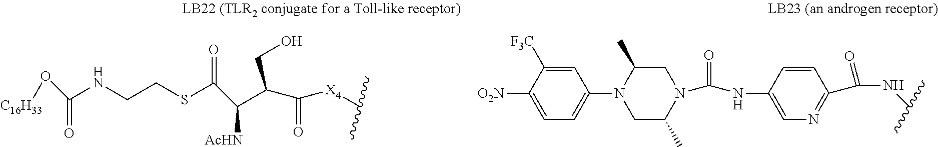

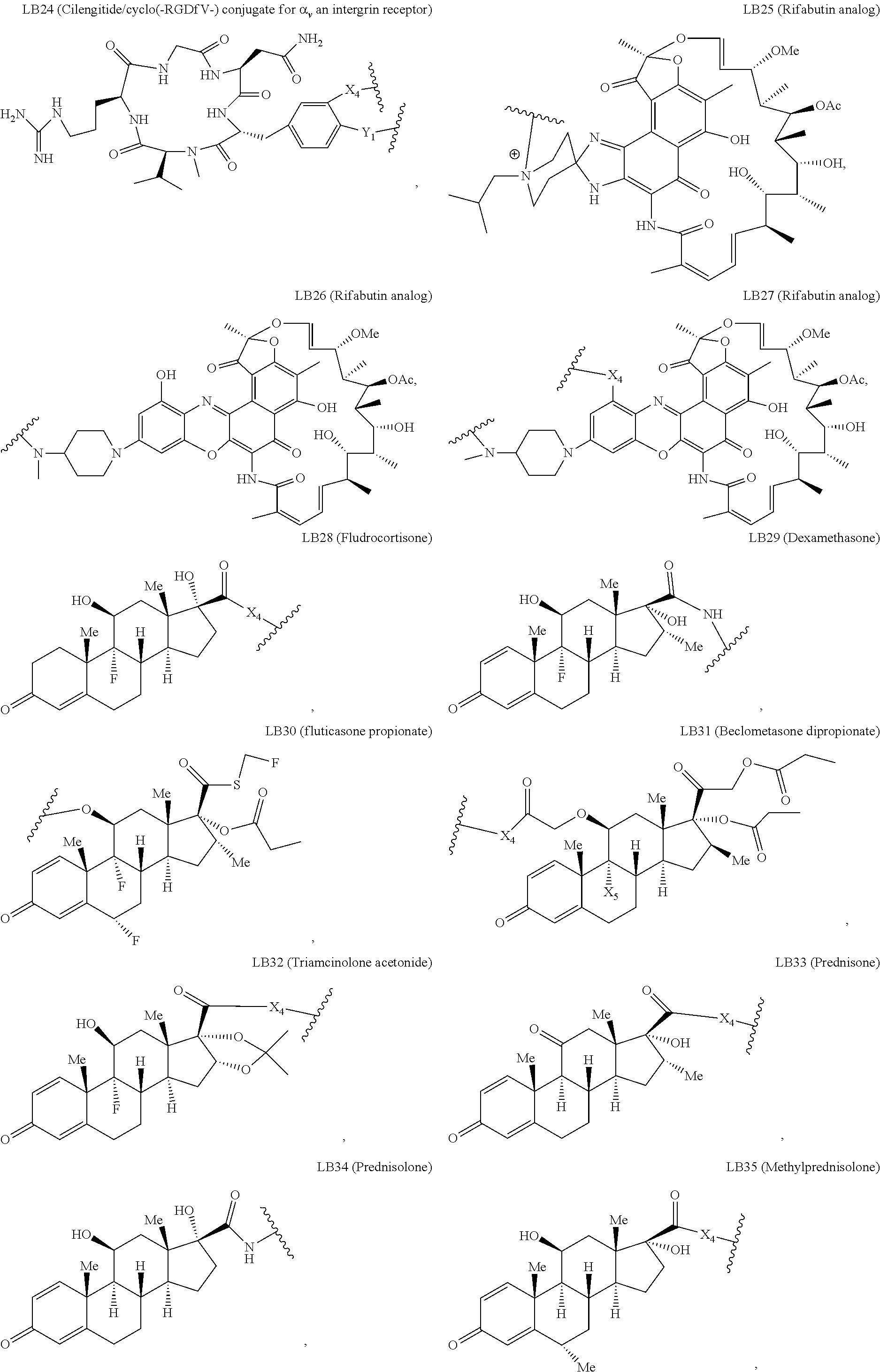

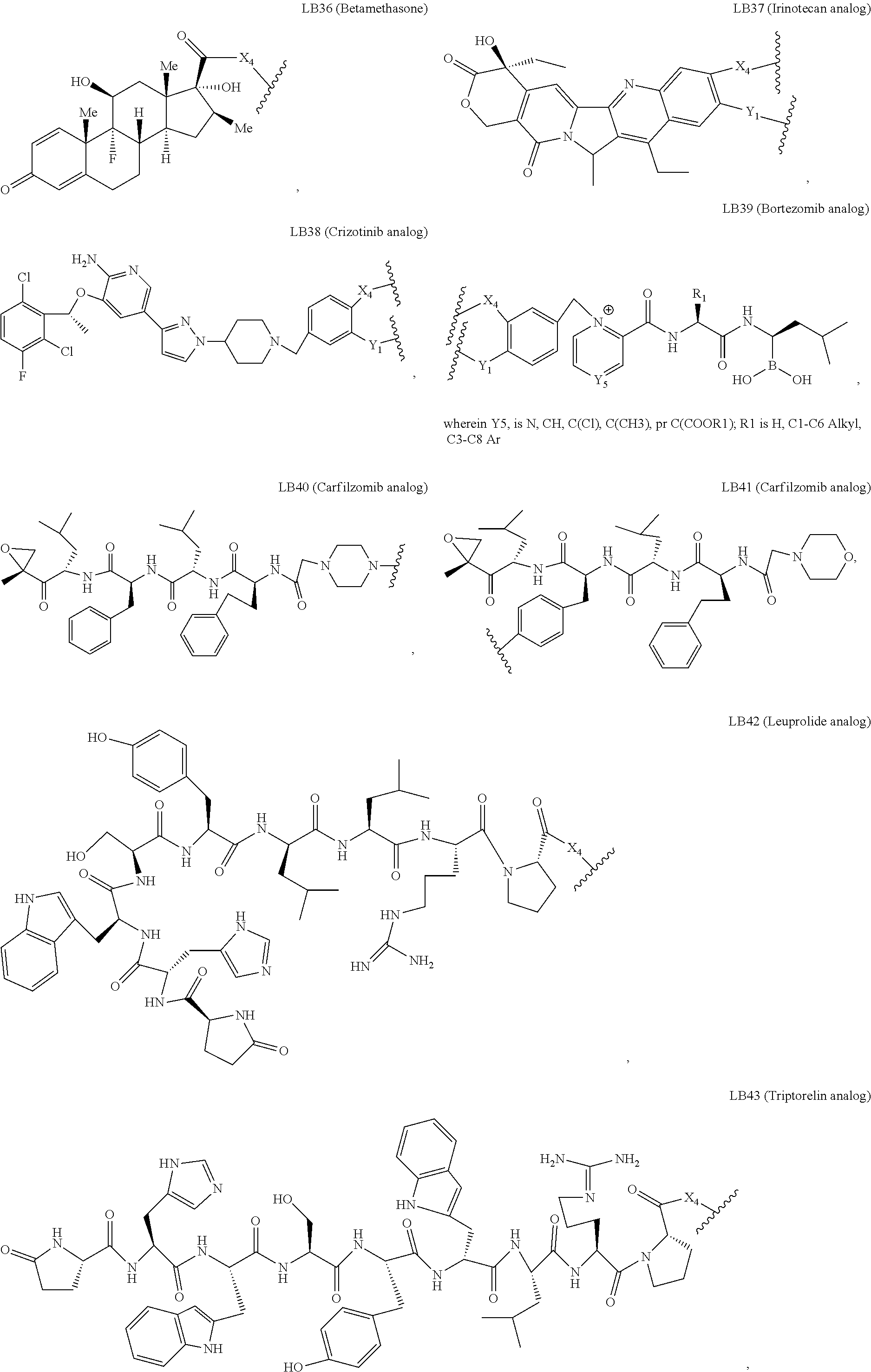

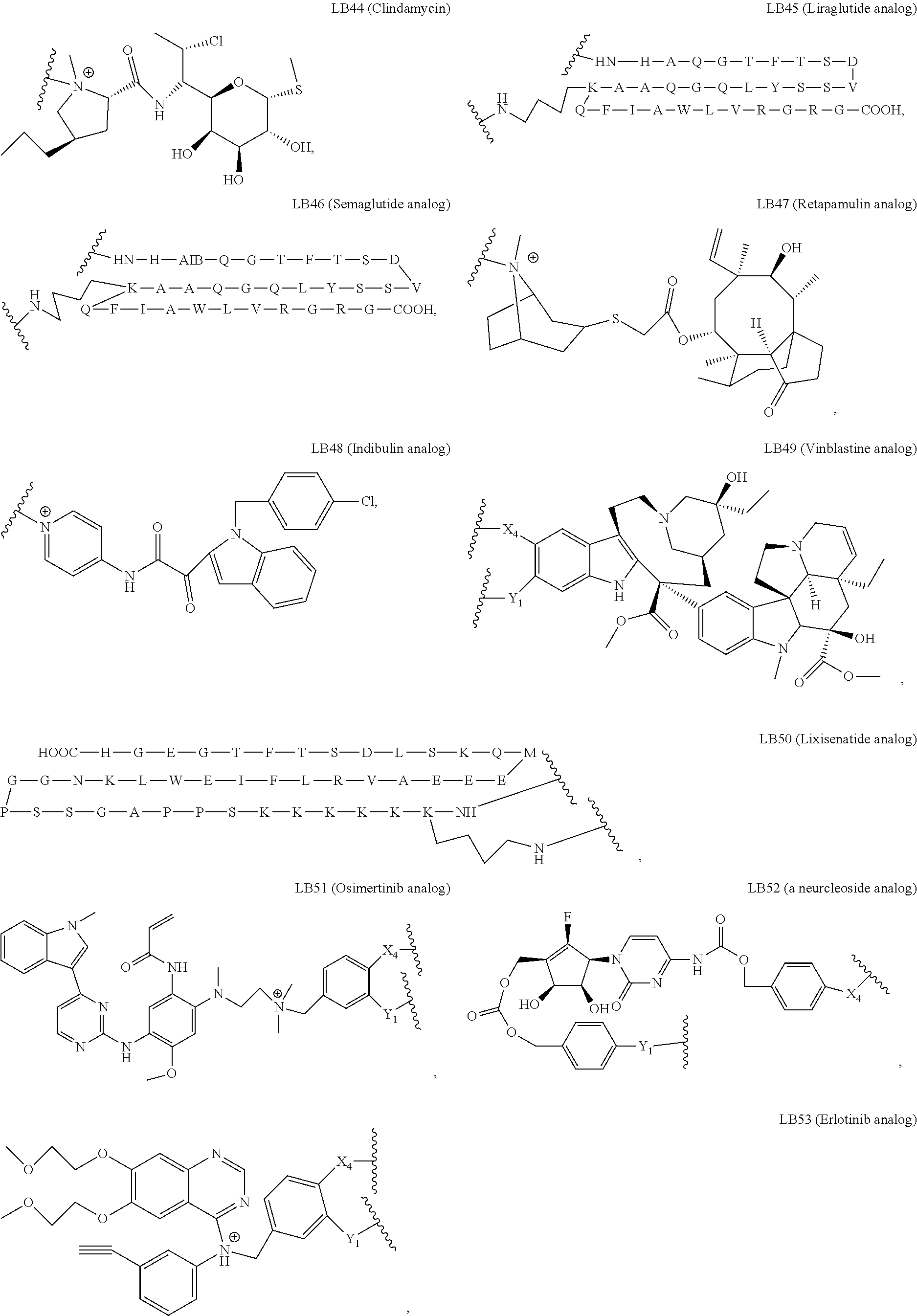

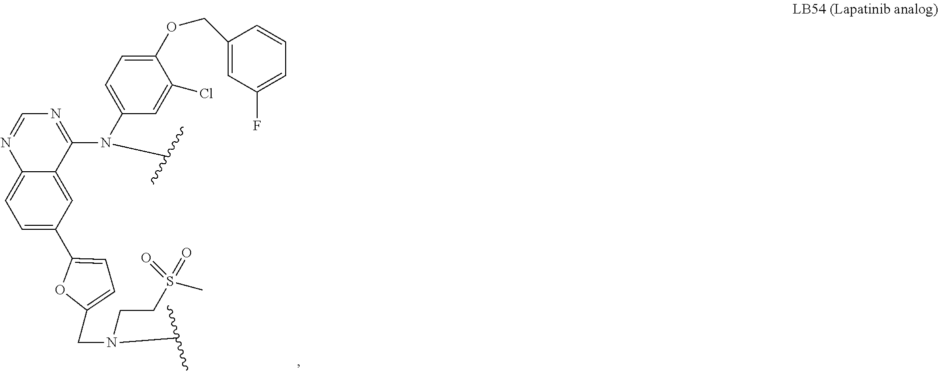 [SMILES: *N(CCS(C)(=O)=O)Cc1ccc(-c2ccc3ncnc(N(*)c4ccc(OCc5cccc(F)c5)c(Cl)c4)c3c2)o1]

wherein “ [symbol] ” is the site to link the side chain linker of the present patent; X₄, and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) and C(O)NR₁; X₁ is H, CH₂, OH, O, C(O), C(O)NH, C(O)N(R₁), R₁, NHR₁, NR₁, C(O)R₁ or C(O)O; X₅ is H, CH₃, F, or Cl; M₁ and M₂ are independently H, Na, K, Ca, Mg, NH₄, N(R₁₂R_{12′}R₁₃ R_{13′}); R₁₂, R_{12′}, R₁₃ and R_{13′} are defined in Formula (I);

In another embodiment, the above ligands can be used as payloads to conjugate to a cell-binding molecule (e. g. an antibody) via the side chain linkers of this application for the targeted treatment or prevention of cancer, infection and autoimmune disease.

In yet another embodiment, one, two or more DNA, RNA, mRNA, small interfering RNA (siRNA), microRNA (miRNA), and PIWI interacting RNAs (piRNA) can be conjugated to a cell-binding molecule via a side-chain linker of this patent. Small RNAs (siRNA, miRNA, piRNA) and long non-coding antisense RNAs are known responsible for epigenetic changes within cells (Goodchild, J (2011), Methods in molecular biology (Clifton, N.J.). 764: 1-15). DNA, RNA, mRNA, siRNA, miRNA or piRNA herein can be single or double strands with nucleotide units from 3 to 1 million and some of their nucleotide can be none natural (synthetic) forms, such as oligonucleotide with phosphorothioate linkage as example of Fomivirsen, or the nucleotides are linked with phosphorothioate linkages rather than the phosphodiester linkages of natural RNA and DNA, and the sugar parts are deoxyribose in the middle part of the molecule and 2′-O-methoxyethyl-modified ribose at the two ends as example Mipomersen, or oligonucleotide made with peptide nucleic acid (PNA), Morpholino, Phosphorothioate, Thiophosphoramidate, or with 2′-O-Methoxyethyl (MOE), 2′-O-Methyl, 2′-Fluoro, Locked Nucleic Acid (LNA), or Bicyclic Nucleic Acid (BNA) of ribose sugar, or nucleic acids are modified to remove the 2′-3′ carbon bond in the sugar ring (Whitehead, K. A.; et al (2011), Annual Review of Chemical and Biomolecular Engineering 2: 77-96; Bennett, C. F.; Swayze, E. E. (2010), Annu. Rev. Pharmacol. Toxicol. 50: 259-29). Preferably, oligonucleotide range in length is from approximately 8 to over 200 nucleotides. Examples of the structures of the nucleotides for conjugates are illustrated below:

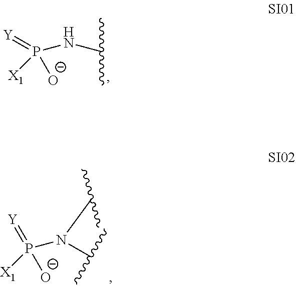

wherein X₁, “ [symbol] ” are the same defined in Formula (I) or above; [symbol] is single or double strands of DNA, RNA, mRNA, siRNA, miRNA, or piRNA; Y is preferably O, S, NH or CH₂.

Application of the Conjugate

In a specific embodiment, the cell-binding ligand-drug conjugates via the side chain linkers of this invention are used for the targeted treatment of cancers. The targeted cancers include, but are not limited, Adrenocortical Carcinoma, Anal Cancer, Bladder Cancer, Brain Tumor (Adult, Brain Stem Glioma, Childhood, Cerebellar Astrocytoma, Cerebral Astrocytoma, Ependymoma, Medulloblastoma, Supratentorial Primitive Neuroectodermal and Pineal Tumors, Visual Pathway and Hypothalamic Glioma), Breast Cancer, Carcinoid Tumor, Gastrointestinal, Carcinoma of Unknown Primary, Cervical Cancer, Colon Cancer, Endometrial Cancer, Esophageal Cancer, Extrahepatic Bile Duct Cancer, Ewings Family of Tumors (PNET), Extracranial Germ Cell Tumor, Eye Cancer, Intraocular Melanoma, Gallbladder Cancer, Gastric Cancer (Stomach), Germ Cell Tumor, Extragonadal, Gestational Trophoblastic Tumor, Head and Neck Cancer, Hypopharyngeal Cancer, Islet Cell Carcinoma, Kidney Cancer (renal cell cancer), Laryngeal Cancer, Leukemia (Acute Lymphoblastic, Acute Myeloid, Chronic Lymphocytic, Chronic Myelogenous, Hairy Cell), Lip and Oral Cavity Cancer, Liver Cancer, Lung Cancer (Non-Small Cell, Small Cell, Lymphoma (AIDS-Related, Central Nervous System, Cutaneous T-Cell, Hodgkin's Disease, Non-Hodgkin's Disease, Malignant Mesothelioma, Melanoma, Merkel Cell Carcinoma, Metasatic Squamous Neck Cancer with Occult Primary, Multiple Myeloma, and Other Plasma Cell Neoplasms, Mycosis Fungoides, Myelodysplastic Syndrome, Myeloproliferative Disorders, Nasopharyngeal Cancer, Neuroblastoma, Oral Cancer, Oropharyngeal Cancer, Osteosarcoma, Ovarian Cancer (Epithelial, Germ Cell Tumor, Low Malignant Potential Tumor), Pancreatic Cancer (Exocrine, Islet Cell Carcinoma), Paranasal Sinus and Nasal Cavity Cancer, Parathyroid Cancer, Penile Cancer, Pheochromocytoma Cancer, Pituitary Cancer, Plasma Cell Neoplasm, Prostate Cancer Rhabdomyosarcoma, Rectal Cancer, Renal Cell Cancer (kidney cancer), Renal Pelvis and Ureter (Transitional Cell), Salivary Gland Cancer, Sezary Syndrome, Skin Cancer, Skin Cancer (Cutaneous T-Cell Lymphoma, Kaposi's Sarcoma, Melanoma), Small Intestine Cancer, Soft Tissue Sarcoma, Stomach Cancer, Testicular Cancer, Thymoma (Malignant), Thyroid Cancer, Urethral Cancer, Uterine Cancer (Sarcoma), Unusual Cancer of Childhood, Vaginal Cancer, Vulvar Cancer, Wilms' Tumor.

In another specific embodiment, the cell-binding-drug conjugates of this invention are used in accordance with the compositions and methods for the treatment or prevention of an autoimmune disease. The autoimmune diseases include, but are not limited, Achlorhydra Autoimmune Active Chronic Hepatitis, Acute Disseminated Encephalomyelitis, Acute hemorrhagic leukoencephalitis, Addison's Disease, Agammaglobulinemia, Alopecia areata, Amyotrophic Lateral Sclerosis, Ankylosing Spondylitis, Anti-GBM/TBM Nephritis, Antiphospholipid syndrome, Antisynthetase syndrome, Arthritis, Atopic allergy, Atopic Dermatitis, Autoimmune Aplastic Anemia, Autoimmune cardiomyopathy, Autoimmune hemolytic anemia, Autoimmune hepatitis, Autoimmune inner ear disease, Autoimmune lymphoproliferative syndrome, Autoimmune peripheral neuropathy, Autoimmune pancreatitis, Autoimmune polyendocrine syndrome Types I, II, & III, Autoimmune progesterone dermatitis, Autoimmune thrombocytopenic purpura, Autoimmune uveitis, Balo disease/Balo concentric sclerosis, Bechets Syndrome, Berger's disease, Bickerstaffs encephalitis, Blau syndrome, Bullous Pemphigoid, Castleman's disease, Chagas disease, Chronic Fatigue Immune Dysfunction Syndrome, Chronic inflammatory demyelinating polyneuropathy, Chronic recurrent multifocal ostomyelitis, Chronic lyme disease, Chronic obstructive pulmonary disease, Churg-Strauss syndrome, Cicatricial Pemphigoid, Coeliac Disease, Cogan syndrome, Cold agglutinin disease, Complement component 2 deficiency, Cranial arteritis, CREST syndrome, Crohns Disease (a type of idiopathic inflammatory bowel diseases), Cushing's Syndrome, Cutaneous leukocytoclastic angiitis, Dego's disease, Dercum's disease, Dermatitis herpetiformis, Dermatomyositis, Diabetes mellitus type 1, Diffuse cutaneous systemic sclerosis, Dressler's syndrome, Discoid lupus erythematosus, Eczema, Endometriosis, Enthesitis-related arthritis, Eosinophilic fasciitis, Epidermolysis bullosa acquisita, Erythema nodosum, Essential mixed cryoglobulinemia, Evan's syndrome, Fibrodysplasia ossificans progressiva, Fibromyalgia, Fibromyositis, Fibrosing aveolitis, Gastritis, Gastrointestinal pemphigoid, Giant cell arteritis, Glomerulonephritis, Goodpasture's syndrome, Graves' disease, Guillain-Barre syndrome, Hashimoto's encephalitis, Hashimoto's thyroiditis, Haemolytic anaemia, Henoch-Schonlein purpura, Herpes gestationis, Hidradenitis suppurativa, Hughes syndrome (See Antiphospholipid syndrome), Hypogamma-globulinemia, Idiopathic Inflammatory Demyelinating Diseases, Idiopathic pulmonary fibrosis, Idiopathic thrombocytopenic purpura (See Autoimmune thrombocytopenic purpura), IgA nephropathy (Also Berger's disease), Inclusion body myositis, Inflammatory demyelinating polyneuopathy, Interstitial cystitis, Irritable Bowel Syndrome, Juvenile idiopathic arthritis, Juvenile rheumatoid arthritis, Kawasaki's Disease, Lambert-Eaton myasthenic syndrome, Leukocytoclastic vasculitis, Lichen planus, Lichen sclerosus, Linear IgA disease (LAD), Lou Gehrig's Disease (Also Amyotrophic lateral sclerosis), Lupoid hepatitis, Lupus erythematosus, Majeed syndrome, Meniere's disease, Microscopic polyangiitis, Miller-Fisher syndrome, Mixed Connective Tissue Disease, Morphea, Mucha-Habermann disease, Muckle-Wells syndrome, Multiple Myeloma, Multiple Sclerosis, Myasthenia gravis, Myositis, Narcolepsy, Neuromyelitis optica (Devic's Disease), Neuromyotonia, Occular cicatricial pemphigoid, Opsoclonus myoclonus syndrome, Ord thyroiditis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria, Parry Romberg syndrome, Parsonnage-Turner syndrome, Pars planitis, Pemphigus, Pemphigus vulgaris, Pernicious anaemia, Perivenous encephalomyelitis, POEMS syndrome, Polyarteritis nodosa, Polymyalgia rheumatica, Polymyositis, Primary biliary cirrhosis, Primary sclerosing cholangitis, Progressive inflammatory neuropathy, Psoriasis, Psoriatic Arthritis, Pyoderma gangrenosum, Pure red cell aplasia, Rasmussen's encephalitis, Raynaud phenomenon, Relapsing polychondritis, Reiter's syndrome, Restless leg syndrome, Retroperitoneal fibrosis, Rheumatoid arthritis, Rheumatoid fever, Sarcoidosis, Schizophrenia, Schmidt syndrome, Schnitzler syndrome, Scleritis, Scleroderma, Sjogren's syndrome, Spondyloarthropathy, Sticky blood syndrome, Still's Disease, Stiff person syndrome, Subacute bacterial endocarditis, Susac's syndrome, Sweet syndrome, Sydenham Chorea, Sympathetic ophthalmia, Takayasu's arteritis, Temporal arteritis (giant cell arteritis), Tolosa-Hunt syndrome, Transverse Myelitis, Ulcerative Colitis (a type of idiopathic inflammatory bowel diseases), Undifferentiated connective tissue disease, Undifferentiated spondyloarthropathy, Vasculitis, Vitiligo, Wegener's granulomatosis, Wilson's syndrome, Wiskott-Aldrich syndrome

In another specific embodiment, a binding molecule used for the conjugate via the side chain-linkers of this invention for the treatment or prevention of an autoimmune disease can be, but are not limited to, anti-elastin antibody; Abys against epithelial cells antibody; Anti-Basement Membrane Collagen Type IV Protein antibody; Anti-Nuclear Antibody; Anti ds DNA; Anti ss DNA, Anti Cardiolipin Antibody IgM, IgG; anti-celiac antibody; Anti Phospholipid Antibody IgK, IgG; Anti SM Antibody; Anti Mitochondrial Antibody; Thyroid Antibody; Microsomal Antibody, T-cells antibody; Thyroglobulin Antibody, Anti SCL-70; Anti-Jo; Anti-U.sub.IRNP; Anti-La/SSB; Anti SSA; Anti SSB; Anti Perital Cells Antibody; Anti Histones; Anti RNP; C-ANCA; P-ANCA; Anti centromere; Anti-Fibrillarin, and Anti GBM Antibody, Anti-ganglioside antibody; Anti-Desmogein 3 antibody; Anti-p62 antibody; Anti-sp100 antibody; Anti-Mitochondrial(M2) antibody; Rheumatoid factor antibody; Anti-MCV antibody; Anti-topoisomerase antibody; Anti-neutrophil cytoplasmic(cANCA) antibody.

In certain preferred embodiments, the binding molecule for the conjugate in the present invention, can bind to both a receptor and a receptor complex expressed on an activated lymphocyte which is associated with an autoimmune disease. The receptor or receptor complex can comprise an immunoglobulin gene superfamily member (e.g. CD2, CD3, CD4, CD8, CD19, CD20, CD22, CD28, CD30, CD33, CD37, CD38, CD56, CD70, CD79, CD79b, CD90, CD125, CD137, CD138, CD147, CD152/CTLA-4, PD-1, or ICOS), a TNF receptor superfamily member (e.g. CD27, CD40, CD95/Fas, CD134/OX40, CD137/4-1BB, INF-R1, TNFR-2, RANK, TACI, BCMA, osteoprotegerin, Apo2/TRAIL-R1, TRAIL-R2, TRAIL-R3, TRAIL-R4, and APO-3), an integrin, a cytokine receptor, a chemokine receptor, a major histocompatibility protein, a lectin (C-type, S-type, or I-type), or a complement control protein.

In another specific embodiment, useful cell binding ligands that are immunospecific for a viral or a microbial antigen are humanized or human monoclonal antibodies. As used herein, the term “viral antigen” includes, but is not limited to, any viral peptide, polypeptide protein (e.g. HIV gp120, HIV nef, RSV F glycoprotein, influenza virus neuramimidase, influenza virus hemagglutinin, HTLV tax, herpes simplex virus glycoprotein (e.g. gB, gC, gD, and gE) and hepatitis B surface antigen) that is capable of eliciting an immune response. As used herein, the term “microbial antigen” includes, but is not limited to, any microbial peptide, polypeptide, protein, saccharide, polysaccharide, or lipid molecule (e.g., a bacteria, fungi, pathogenic protozoa, or yeast polypeptides including, e.g., LPS and capsular polysaccharide 5/8) that is capable of eliciting an immune response. Examples of antibodies available 1 for the viral or microbial infection include, but are not limited to, Palivizumab which is a humanized anti-respiratory syncytial virus monoclonal antibody for the treatment of RSV infection; PRO542 which is a CD4 fusion antibody for the treatment of HIV infection; Ostavir which is a human antibody for the treatment of hepatitis B virus; PROTVIR which is a humanized IgG.sub.1 antibody for the treatment of cytomegalovirus; and anti-LPS antibodies.

The cell binding molecules-drug conjugates via the side chain-linkers of this invention can be used in the treatment of infectious diseases. These infectious diseases include, but are not limited to, Acinetobacter infections, Actinomycosis, African sleeping sickness (African trypanosomiasis), AIDS (Acquired immune deficiency syndrome), Amebiasis, Anaplasmosis, Anthrax, Arcano-bacterium haemolyticum infection, Argentine hemorrhagic fever, Ascariasis, Aspergillosis, Astrovirus infection, Babesiosis, Bacillus cereus infection, Bacterial pneumonia, Bacterial vaginosis, Bacteroides infection, Balantidiasis, Baylisascaris infection, BK virus infection, Black piedra, Blastocystis hominis infection, Blastomycosis, Bolivian hemorrhagic fever, Borrelia infection, Botulism (and Infant botulism), Brazilian hemorrhagic fever, Brucellosis, Burkholderia infection, Buruli ulcer, Calicivirus infection (Norovirus and Sapovirus), Campylobacteriosis, Candidiasis (Moniliasis; Thrush), Cat-scratch disease, Cellulitis, Chagas Disease (American trypanosomiasis), Chancroid, Chickenpox, Chlamydia, Chlamydophila pneumoniae infection, Cholera, Chromoblastomycosis, Clonorchiasis, Clostridium difficile infection, Coccidioido-mycosis, Colorado tick fever, Common cold (Acute viral rhinopharyngitis; Acute coryza), Creutzfeldt-Jakob disease, Crimean-Congo hemorrhagic fever, Cryptococcosis, Cryptosporidiosis, Cutaneous larva migrans, Cyclosporiasis, Cysticercosis, Cytomegalovirus infection, Dengue fever, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum, Fasciolopsiasis, Fasciolosis, Fatal familial insomnia, Filariasis, Food poisoning by Clostridium perfringens, Free-living amebic infection, Fusobacterium infection, Gas gangrene (Clostridial myonecrosis), Geotrichosis, Gerstmann-Straussler-Scheinker syndrome, Giardiasis, Glanders, Gnathosto-miasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome, Helicobacter pylori infection, Hemolytic-uremic syndrome, Hemorrhagic fever with renal syndrome, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis, Human metapneumovirus infection, Human monocytic ehrlichiosis, Human papillomavirus infection, Human parainfluenza virus infection, Hymenolepiasis, Epstein-Barr Virus Infectious Mononucleosis (Mono), Influenza, Isosporiasis, Kawasaki disease, Keratitis, Kingella kingae infection, Kuru, Lassa fever, Legionellosis (Legionnaires' disease), Legionellosis (Pontiac fever), Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever, Measles, Melioidosis (Whitmore's disease), Meningitis, Meningococcal disease, Metagonimiasis, Microsporidiosis, Molluscum contagiosum, Mumps, Murine typhus (Endemic typhus), Mycoplasma pneumonia, Mycetoma, Myiasis, Neonatal conjunctivitis (Ophthalmia neonatorum), (New) Variant Creutzfeldt-Jakob disease (vCJD, nvCJD), Nocardiosis, Onchocerciasis (River blindness), Paracoccidioidomycosis (South American blastomycosis), Paragonimiasis, Pasteurellosis, Pediculosis capitis (Head lice), Pediculosis corporis (Body lice), Pediculosis pubis (Pubic lice, Crab lice), Pelvic inflammatory disease, Pertussis (Whooping cough), Plague, Pneumococcal infection, Pneumocystis pneumonia, Pneumonia, Poliomyelitis, Prevotella infection, Primary amoebic meningoencephalitis, Progressive multifocal leukoencephalopathy, Psittacosis, Q fever, Rabies, Rat-bite fever, Respiratory syncytial virus infection, Rhinosporidiosis, Rhinovirus infection, Rickettsial infection, Rickettsial-pox, Rift Valley fever, Rocky mountain spotted fever, Rotavirus infection, Rubella, Salmonellosis, SARS (Severe Acute Respiratory Syndrome), Scabies, Schistosomiasis, Sepsis, Shigellosis (Bacillary dysentery), Shingles (Herpes zoster), Smallpox (Variola), Sporotrichosis, Staphylococcal food poisoning, Staphylococcal infection, Strongyloidiasis, Syphilis, Taeniasis, Tetanus (Lockjaw), Tinea barbae (Barber's itch), Tinea capitis (Ringworm of the Scalp), Tinea corporis (Ringworm of the Body), Tinea cruris (Jock itch), Tinea manuum (Ringworm of the Hand), Tinea nigra, Tinea pedis (Athlete's foot), Tinea unguium (Onychomycosis), Tinea versicolor (Pityriasis versicolor), Toxocariasis (Ocular Larva Migrans), Toxocariasis (Visceral Larva Migrans), Toxoplasmosis, Trichinellosis, Trichomoniasis, Trichuriasis (Whipworm infection), Tuberculosis, Tularemia, Ureaplasma urealyticum infection, Venezuelan equine encephalitis, Venezuelan hemorrhagic fever, Viral pneumonia, West Nile Fever, White piedra (Tinea blanca), Yersinia pseudotuberculosis infection, Yersiniosis, Yellow fever, Zygomycosis.

The conjugate of the invention is further preferred to be able to against pathogenic strains including, but are not limit, Acinetobacter baumannii, Actinomyces israelii, Actinomyces gerencseriae and Propionibacterium propionicus, Trypanosoma brucei, HIV (Human immunodeficiency virus), Entamoeba histolytica, Anaplasma genus, Bacillus anthracis, Arcanobacterium haemolyticum, Junin virus, Ascaris lumbricoides, Aspergillus genus, Astroviridae family, Babesia genus, Bacillus cereus, multiple bacteria, Bacteroides genus, Balantidium coli, Baylisascaris genus, BK virus, Piedraia hortae, Blastocystis hominis, Blastomyces dermatitides, Machupo virus, Borrelia genus, Clostridium botulinum, Sabia, Brucella genus, usually Burkholderia cepacia and other Burkholderia species, Mycobacterium ulcerans, Caliciviridae family, Campylobacter genus, usually Candida albicans and other Candida species, Bartonella henselae, Group A Streptococcus and Staphylococcus, Trypanosoma cruzi, Haemophilus ducreyi, Varicella zoster virus (VZV), Chlamydia trachomatis, Chlamydophila pneumoniae, Vibrio cholerae, Fonsecaea pedrosoi, Clonorchis sinensis, Clostridium difficile, Coccidioides immitis and Coccidioides posadasii, Colorado tick fever virus, rhinoviruses, coronaviruses, CJD prion, Crimean-Congo hemorrhagic fever virus, Cryptococcus neoformans, Cryptosporidium genus, Ancylostoma braziliense; multiple parasites, Cyclospora cayetanensis, Taenia solium, Cytomegalovirus, Dengue viruses (DEN-1, DEN-2, DEN-3 and DEN-4)—Flaviviruses, Dientamoeba fragilis, Corynebacterium diphtheriae, Diphyllobothrium, Dracunculus medinensis, Ebolavirus, Echinococcus genus, Ehrlichia genus, Enterobius vermicularis, Enterococcus genus, Enterovirus genus, Rickettsia prowazekii, Parvovirus B19, Human herpesvirus 6 and Human herpesvirus 7, Fasciolopsis buski, Fasciola hepatica and Fasciola gigantica, FFI prion, Filarioidea superfamily, Clostridium perfringens, Fusobacterium genus, Clostridium perfringens; other Clostridium species, Geotrichum candidum, GSS prion, Giardia intestinalis, Burkholderia mallei, Gnathostoma spinigerum and Gnathostoma hispidum, Neisseria gonorrhoeae, Klebsiella granulomatis, Streptococcus pyogenes, Streptococcus agalactiae, Haemophilus influenzae, Enteroviruses, mainly Coxsackie A virus and Enterovirus 71, Sin Nombre virus, Helicobacter pylori, Escherichia coli O157.H7, Bunyaviridae family, Hepatitis A Virus, Hepatitis B Virus, Hepatitis C Virus, Hepatitis D Virus, Hepatitis E Virus, Herpes simplex virus 1, Herpes simplex virus 2, Histoplasma capsulatum, Ancylostoma duodenale and Necator americanus, Hemophilus influenzae, Human bocavirus, Ehrlichia ewingii, Anaplasma phagocytophilum, Human metapneumovirus, Ehrlichia chaffeensis, Human papillomavirus, Human parainfluenza viruses, Hymenolepis nana and Hymenolepis diminuta, Epstein-Barr Virus, Orthomy-xoviridae family, Isospora belli, Kingella kingae, Klebsiella pneumoniae, Klebsiella ozaenas, Klebsiella rhinoscleromotis, Kuru prion, Lassa virus, Legionella pneumophila, Legionella pneumophila, Leishmania genus, Mycobacterium leprae and Mycobacterium lepromatosis, Leptospira genus, Listeria monocytogenes, Borrelia burgdorferi and other Borrelia species, Wuchereria bancrofti and Brugia malayi, Lymphocytic choriomeningitis virus (LCMV), Plasmodium genus, Marburg virus, Measles virus, Burkholderia pseudomallei, Neisseria meningitides, Metagonimus yokagawai, Microsporidia phylum, Molluscum contagiosum virus (MCV), Mumps virus, Rickettsia typhi, Mycoplasma pneumoniae, numerous species of bacteria (Actinomycetoma) and fungi (Eumycetoma), parasitic dipterous fly larvae, Chlamydia trachomatis and Neisseria gonorrhoeae, vCJD prion, Nocardia asteroides and other Nocardia species, Onchocerca volvulus, Paracoccidioides brasiliensis, Paragonimus westermani and other Paragonimus species, Pasteurella genus, Pediculus humanus capitis, Pediculus humanus corporis, Phthirus pubis, Bordetella pertussis, Yersinia pestis, Streptococcus pneumoniae, Pneumocystis jirovecii, Poliovirus, Prevotella genus, Naegleria fowleri, JC virus, Chlamydophila psittaci, Coxiella burnetii, Rabies virus, Streptobacillus moniliformis and Spirillum minus, Respiratory syncytial virus, Rhinosporidium seeberi, Rhinovirus, Rickettsia genus, Rickettsia akari, Rift Valley fever virus, Rickettsia rickettsii, Rotavirus, Rubella virus, Salmonella genus, SARS coronavirus, Sarcoptes scabiei, Schistosoma genus, Shigella genus, Varicella zoster virus, Variola major or Variola minor, Sporothrix schenckii, Staphylococcus genus, Staphylococcus genus, Staphylococcus aureus, Streptococcus pyogenes, Strongyloides stercoralis, Treponema pallidum, Taenia genus, Clostridium tetani, Trichophyton genus, Trichophyton tonsurans, Trichophyton genus, Epidermophyton floccosum, Trichophyton rubrum, and Trichophyton mentagrophytes, Trichophyton rubrum, Hortaea werneckii, Trichophyton genus, Malassezia genus, Toxocara canis or Toxocara cati, Toxoplasma gondii, Trichinella spiralis, Trichomonas vaginalis, Trichuris trichiura, Mycobacterium tuberculosis, Francisella tularensis, Ureaplasma urealyticum, Venezuelan equine encephalitis virus, Vibrio colerae, Guanarito virus, West Nile virus, Trichosporon beigelii, Yersinia pseudotuberculosis, Yersinia enterocolitica, Yellow fever virus, Mucorales order (Mucormycosis) and Entomophthorales order (Entomophthora-mycosis), Pseudomonas aeruginosa, Campylobacter (Vibrio) fetus, Aeromonas hydrophila, Edwardsiella tarda, Yersinia pestis, Shigella dysenteriae, Shigella flexneri, Shigella sonnei, Salmonella typhimurium, Treponema pertenue, Treponema carateneum, Borrelia vincentii, Borrelia burgdorferi, Leptospira icterohemorrhagiae, Pneumocystis carinii, Brucella abortus, Brucella suis, Brucella melitensis, Mycoplasma spp., Rickettsia prowazeki, Rickettsia tsutsugumushi, Clamydia spp.; pathogenic fungi (Aspergillus fumigatus, Candida albicans, Histoplasma capsulatum); protozoa (Entomoeba histolytica, Trichomonas tenas, Trichomonas hominis, Tryoanosoma gambiense, Trypanosoma rhodesiense, Leishmania donovani, Leishmania tropica, Leishmania braziliensis, Pneumocystis pneumonia, Plasmodium vivax, Plasmodium falciparum, Plasmodium malaria); or Helminiths (Schistosoma japonicum, Schistosoma mansoni, Schistosoma haematobium, and hookworms).

Further conjugates of this invention are for treatment of viral disease which include, but are not limited to, pathogenic viruses, such as, Poxyiridae, Herpesviridae, Adenoviridae, Papovaviridae, Enteroviridae, Picornaviridae, Parvoviridae, Reoviridae, Retroviridae, influenza viruses, parainfluenza viruses, mumps, measles, respiratory syncytial virus, rubella, Arboviridae, Rhabdoviridae, Arenaviridae, Non-A/Non-B Hepatitis virus, Rhinoviridae, Coronaviridae, Rotoviridae, Oncovirus [such as, HBV (Hepatocellular carcinoma), HPV (Cervical cancer, Anal cancer), Kaposi's sarcoma-associated herpesvirus (Kaposi's sarcoma), Epstein-Barr virus (Nasopharyngeal carcinoma, Burkitt's lymphoma, Primary central nervous system lymphoma), MCPyV (Merkel cell cancer), SV40 (Simian virus 40), HCV (Hepatocellular carcinoma), HTLV-I (Adult T-cell leukemia/lymphoma)], Immune disorders caused virus: [such as Human Immunodeficiency Virus (AIDS)]; Central nervous system virus: [such as, JCV (Progressive multifocal leukoencephalopathy), MeV (Subacute sclerosing panencephalitis), LCV (Lymphocytic choriomeningitis), Arbovirus encephalitis, Orthomyxoviridae (probable) (Encephalitis lethargica), RV (Rabies), Chandipura virus, Herpesviral meningitis, Ramsay Hunt syndrome type II; Poliovirus (Poliomyelitis, Post-polio syndrome), HTLV-I (Tropical spastic paraparesis)]; Cytomegalovirus (Cytomegalovirus retinitis, HSV (Herpetic keratitis)); Cardiovascular virus [such as CBV (Pericarditis, Myocarditis)]; Respiratory system/acute viral nasopharyngitis/viral pneumonia: [Epstein-Barr virus (EBV infection/Infectious mononucleosis), Cytomegalovirus; SARS coronavirus (Severe acute respiratory syndrome) Orthomyxoviridae: Influenzavirus A/B/C (Influenza/Avian influenza), Paramyxovirus: Human parainfluenza viruses (Parainfluenza), RSV (Human respiratory syncytialvirus), hMPV]; Digestive system virus [MuV (Mumps), Cytomegalovirus (Cytomegalovirus esophagitis); Adenovirus (Adenovirus infection); Rotavirus, Norovirus, Astrovirus, Coronavirus; HBV (Hepatitis B virus), CBV, HAV (Hepatitis A virus), HCV (Hepatitis C virus), HDV (Hepatitis D virus), HEV (Hepatitis E virus), HGV (Hepatitis G virus)]; Urogenital virus [such as, BK virus, MuV (Mumps)].

According to a further object, the present invention also concerns pharmaceutical compositions comprising the conjugate of the invention together with a pharmaceutically acceptable carrier, diluent, or excipient for treatment of cancers, infections or autoimmune disorders. The method for treatment of cancers, infections and autoimmune disorders can be practiced in vitro, in vivo, or ex vivo. Examples of in vitro uses include treatments of cell cultures in order to kill all cells except for desired variants that do not express the target antigen; or to kill variants that express undesired antigen. Examples of ex vivo uses include treatments of hematopoietic stem cells (HSC) prior to the performance of the transplantation (HSCT) into the same patient in order to kill diseased or malignant cells. For instance, clinical ex vivo treatment to remove tumour cells or lymphoid cells from bone marrow prior to autologous transplantation in cancer treatment or in treatment of autoimmune disease, or to remove T cells and other lymphoid cells from allogeneic bone marrow or tissue prior to transplant in order to prevent graft-versus-host disease, can be carried out as follows. Bone marrow is harvested from the patient or other individual and then incubated in medium containing serum to which is added the conjugate of the invention, concentrations range from about 1 pM to 0.1 mM, for about 15 minutes to about 48 hours at about 37° C. The exact conditions of concentration and time of incubation (=dose) are readily determined by the skilled clinicians. After incubation, the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

A Chemotheropeutic Drug/Cytotoxic Agent for Synergy or are for Use as a Payload for a Conjugate with the Linker

Chemotheropeutic drug that can be used as a payload for conjugation of the present invention or along with a conjugate of the present invention for synergic treatment are small molecule drugs including cytotoxic agents. A “small molecule drug” is broadly used herein to refer to an organic, inorganic, or organometallic compound that may have a molecular weight of, for example, 100 to 2500, more suitably from 200 to 2000. Small molecule drugs are well characterized in the art, such as in WO05058367A2, U.S. Pat. No. 4,956,303, and in: Chessum, N., et al, Prog Med Chem. 2015, 54: 1-63; Eder, J., et al, Nat Rev Drug Discov. 2014, 13(8): 577-87; Zhang, M.-Q., et al, Curr Opin Biotechnol. 2007, 18(6): 478-88; among others and are incorporated in their entirety by reference. The drugs include known drugs and those that may become known drugs.

A cytotoxic drug that is known includes, but not limited to,

1). Chemotherapeutic agents: a). Alkylating agents: such as Nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 (including its adozelesin, carzelesin and bizelesin synthetic analogues); Duocarmycin (including the synthetic analogues, KW-2189 and CBI-TMI); Benzodiazepine dimers (e.g., dimmers of pyrrolobenzodiazepine (PBD) or tomaymycin, indolinobenzodiazepines, imidazobenzothiadiazepines, or oxazolidino-benzodiazepines); Nitrosoureas: (carmustine, lomustine, chlorozotocin, fotemustine, nimustine, ranimustine); Alkylsulphonates: (busulfan, treosulfan, improsulfan and piposulfan); Triazenes: (dacarbazine); Platinum containing compounds: (carboplatin, cisplatin, oxaliplatin); aziridines, such as benzodopa, carboquone, meturedopa, and uredopa; ethylenimines and methylamelamines including altretamine, triethylenemel-amine, trietylenephosphoramide, triethylenethio-phosphaoramide and trimethylolomel-amine]; b). Plant Alkaloids: such as Vinca alkaloids: (vincristine, vinblastine, vindesine, vinorelbine, navelbin); Taxoids: (paclitaxel, docetaxol) and their analogs, Maytansinoids (DM1, DM2, DM3, DM4, maytansine and ansamitocins) and their analogs, cryptophycins (particularly cryptophycin 1 and cryptophycin 8); epothilones, eleutherobin, discodermolide, bryostatins, dolostatins, auristatins, amatoxins, cephalostatins; pancratistatin; a sarcodictyin; spongistatin; c). DNA Topoisomerase Inhibitors: such as [Epipodophyllins: (9-aminocamptothecin, camptothecin, crisnatol, daunomycin, etoposide, etoposide phosphate, irinotecan, mitoxantrone, novantrone, retinoic acids (retinols), teniposide, topotecan, 9-nitrocamptothecin (RFS 2000)); mitomycins: (mitomycin C)]; d). Anti-metabolites: such as {[Anti-folate: DHFR inhibitors: (methotrexate, trimetrexate, denopterin, pteropterin, aminopterin (4-aminopteroic acid) or the other folic acid analogues); IMP dehydrogenase Inhibitors: (mycophenolic acid, tiazofurin, ribavirin, EICAR); Ribonucleotide reductase Inhibitors: (hydroxyurea, deferoxamine)]; [Pyrimidine analogs: Uracil analogs: (ancitabine, azacitidine, 6-azauridine, capecitabine (Xeloda), carmofur, cytarabine, dideoxyuridine, doxifluridine, enocitabine, 5-Fluorouracil, floxuridine, ratitrexed (Tomudex)); Cytosine analogs: (cytarabine, cytosine arabinoside, fludarabine); Purine analogs: (azathioprine, fludarabine, mercaptopurine, thiamiprine, thioguanine)]; folic acid replenisher, such as frolinic acid}; e). Hormonal therapies: such as {Receptor antagonists: [Anti-estrogen: (megestrol, raloxifene, tamoxifen); LHRH agonists: (goscrclin, leuprolide acetate); Anti-androgens: (bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (CB 1093, EB 1089 KH 1060, cholecalciferol, ergocalciferol); Photodynamic therapies: (verteporfin, phthalocyanine, photosensitizer Pc4, demethoxy-hypocrellin A); Cytokines: (Interferon-alpha, Interferon-gamma, tumor necrosis factor (TNFs), human proteins containing a TNF domain)]}; f). Kinase inhibitors, such as BIBW 2992 (anti-EGFR/Erb2), imatinib, gefitinib, pegaptanib, sorafenib, dasatinib, sunitinib, erlotinib, nilotinib, lapatinib, axitinib, pazopanib. vandetanib, E7080 (anti-VEGFR2), mubritinib, ponatinib (AP24534), bafetinib (INNO-406), bosutinib (SKI-606), cabozantinib, vismodegib, iniparib, ruxolitinib, CYT387, axitinib, tivozanib, sorafenib, bevacizumab, cetuximab, Trastuzumab, Ranibizumab, Panitumumab, ispinesib; g). A poly (ADP-ribose) polymerase (PARP) inhibitors, such as olaparib, niraparib, iniparib, talazoparib, veliparib, veliparib, CEP 9722 (Cephalon's), E7016 (Eisai's), BGB-290 (BeiGene's), 3-aminobenzamide.

h). antibiotics, such as the enediyne antibiotics (e.g. calicheamicins, especially calicheamicin γ1, δ1, α1 and β1, see, e.g., J. Med. Chem., 39 (11), 2103-2117 (1996), Angew Chem Intl. Ed. Engl. 33:183-186 (1994); dynemicin, including dynemicin A and deoxydynemicin; esperamicin, kedarcidin, C-1027, maduropeptin, as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; i). Others: such as Polyketides (acetogenins), especially bullatacin and bullatacinone; gemcitabine, epoxomicins (e. g. carfilzomib), bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors (such as Lovastatin), Dopaminergic neurotoxins (such as 1-methyl-4-phenylpyridinium ion), Cell cycle inhibitors (such as staurosporine), Actinomycins (such as Actinomycin D, dactinomycin), Bleomycins (such as bleomycin A2, bleomycin B2, peplomycin), Anthracyclines (such as daunorubicin, doxorubicin (adriamycin), idarubicin, epirubicin, pirarubicin, zorubicin, mtoxantrone, MDR inhibitors (such as verapamil), Ca²⁺ ATPase inhibitors (such as thapsigargin), Histone deacetylase inhibitors (Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A.; Anti-adrenals, such as aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK©; razoxane; rhizoxin; sizofiran; spirogermanium; tenuazonic acid; triaziquone; 2, 2′,2″-trichlorotriethylamine; trichothecenes (especially T-2 toxin, verrucarin A, roridin A and anguidine); urethane, siRNA, antisense drugs, and a nucleolytic enzyme.

2). An anti-autoimmune disease agent includes, but is not limited to, cyclosporine, cyclosporine A, aminocaproic acid, azathioprine, bromocriptine, chlorambucil, chloroquine, cyclophosphamide, corticosteroids (e.g. amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus.

3). An anti-infectious disease agent includes, but is not limited to, a). Aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin; b). Amphenicols: azidamfenicol, chloramphenicol, florfenicol, thiamphenicol; c). Ansamycins: geldanamycin, herbimycin; d). Carbapenems: biapenem, doripenem, ertapenem, imipenem/cilastatin, meropenem, panipenem; e). Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (cefoxitin, cefotetan, cefmetazole), oxacephem (flomoxef, latamoxef); f). Glycopeptides: bleomycin, vancomycin (oritavancin, telavancin), teicoplanin (dalbavancin), ramoplanin; g). Glycylcyclines: e. g. tigecycline; g). β-Lactamase inhibitors: penam (sulbactam, tazobactam), clavam (clavulanic acid); i). Lincosamides: clindamycin, lincomycin; j). Lipopeptides: daptomycin, A54145, calcium-dependent antibiotics (CDA); k). Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin; l). Monobactams: aztreonam, tigemonam; m). Oxazolidinones: linezolid; n). Penicillins: amoxicillin, ampicillin (pivampicillin, hetacillin, bacampicillin, metampicillin, talampicillin), azidocillin, azlocillin, benzylpenicillin, benzathine benzylpenicillin, benzathine phenoxymethyl-penicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin; o). Polypeptides: bacitracin, colistin, polymyxin B; p). Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin; q). Streptogramins: pristinamycin, quinupristin/dalfopristin); r). Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole); s). Steroid antibacterials: e.g. fusidic acid; t). Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (e.g. tigecycline); u). Other types of antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (e. g. fosfomycin), nitrofurantoin, paclitaxel, platensimycin, pyrazinamide, quinupristin/dalfopristin, rifampicin (rifampin), tazobactam tinidazole, uvaricin;

4). Anti-viral drugs: a). Entry/fusion inhibitors: aplaviroc, maraviroc, vicriviroc, gp41 (enfuvirtide), PRO 140, CD4 (ibalizumab); b). Integrase inhibitors: raltegravir, elvitegravir, globoidnan A; c). Maturation inhibitors: bevirimat, vivecon; d). Neuraminidase inhibitors: oseltamivir, zanamivir, peramivir; e). Nucleosides &nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′, 3′-dideoxynucleoside analogues (e.g. 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (e.g. β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT); f). Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine; g). Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir; h). Other types of anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib.

5). The radioisotopes for radiotherapy. Examples of radioisotopes (radionuclides) are ³H, ¹¹C, ¹⁴C, ¹⁸F, ³²P, ³⁵S, ⁶⁴Cu, ⁶⁸Ga, ⁸⁶Y, ⁹⁹Tc, ¹¹¹In, ¹²³I, ¹²⁴I, ¹²⁵I, ¹³¹I, ¹³³Xe, ¹⁷⁷Lu, ²¹¹At, or ²¹³Bi. Radioisotope labeled antibodies are useful in receptor targeted imaging experiments or can be for targeted treatment such as with the antibody-radioisotope conjugates (Wu et al (2005) Nature Biotechnology 23(9): 1137-46). The cell binding molecules, e.g. an antibody can be labeled with ligand reagents that bind, chelate or otherwise complex a radioisotope metal, using the techniques described in Current Protocols in Immunology, Volumes 1 and 2, Coligen et al, Ed. Wiley-Interscience, New York, Pubs. (1991). Chelating ligands which may complex a metal ion include DOTA, DOTP, DOTMA, DTPA and TETA (Macrocyclics, Dallas, Tex. USA).

6). Another cell-binding molecule-drug conjugate as a synergy therapy. The preferred synergic conjugate can be a conjugate having a cytotoxic agent of a tubulysin analog, maytansinoid analog, taxanoid (taxane) analog, CC-1065 analog, daunorubicin and doxorubicin compound, amatoxin analog, benzodiazepine dimer (e.g., dimers of pyrrolobenzodiazepine (PBD), tomaymycin, anthramycin, indolinobenzodiazepines, imidazobenzothiadiazepines, or oxazolidinobenzodiazepines), calicheamicins and the enediyne antibiotic compound, actinomycin, azaserine, bleomycins, epirubicin, tamoxifen, idarubicin, dolastatins, auristatins (e.g. monomethyl auristatin E, MMAE, MMAF, auristatin PYE, auristatin TP, Auristatins 2-AQ, 6-AQ, EB (AEB), and EFP (AEFP)), duocarmycins, geldanamycins, methotrexates, thiotepa, vindesines, vincristines, hemiasterlins, nazumamides, microginins, radiosumins, alterobactins, microsclerodermins, theonellamides, esperamicins, PNU-159682, and their analogues and derivatives above thereof.

7). The pharmaceutically acceptable salts, acids or derivatives of any of the above drugs.

In yet another embodiment, an immunotoxin can be conjugated to a cell-binding molecule via the linkers of the invention. An immunotoxin herein is a macromolecular drug which is usually a cytotoxic protein derived from a bacterial or plant protein, such as Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, etc. It also can be a highly toxic bacterial pore-forming protoxin that requires proteolytic processing for activation. An example of this protoxin is proaerolysin and its genetically modified form, topsalysin. Topsalysin is a modified recombinant protein that has been engineered to be selectively activated by an enzyme in the prostate, leading to localized cell death and tissue disruption without damaging neighboring tissue and nerves.

In another synergistic immunotherapy, an antibody of a checkpoint inhibitor, TCR (T cell receptors) T cells, or CARs (chimeric antigen receptors) T cells, or of B cell receptor (BCR), Natural killer (NK) cells, or the cytotoxic cells, or an antibody of anti-CD3, CD4, CD8, CD16 (FcγRIII), CD19, CD20, CD22, CD25, CD27, CD30, CD33, CD37, CD38, CD40, CD40L, CD45RA, CD45RO, CD56, CD57, CD57^{bright}, CD70, CD79, CD79b, CD123, CD125, CD138, TNFβ, Fas ligand, MHC class I molecules (HLA-A, B, C), VEGF, or NKR-P1 is preferred to use along with the conjugates of the present patent for synergistic therapy.

Formulation and Application

The conjugates of the patent application are formulated to liquid, or suitable to be lyophilized and subsequently be reconstituted to a liquid formulation. The conjugate in a liquid formula or in the formulated lyophilized powder may take up 0.01%-99% by weight as major gradient in the formulation. In general, a liquid formulation comprising 0.1 g/L˜300 g/L of concentration of the conjugate active ingredient for delivery to a patient without high levels of antibody aggregation may include one or more polyols (e.g. sugars), a buffering agent with pH 4.5 to 7.5, a surfactant (e.g. polysorbate 20 or 80), an antioxidant (e.g. ascorbic acid and/or methionine), a tonicity agent (e.g. mannitol, sorbitol or NaCl), chelating agents such as EDTA; metal complexes (e.g. Zn-protein complexes); biodegradable polymers such as polyesters; a preservative (e.g. benzyl alcohol) and/or a free amino acid.

Suitable buffering agents for use in the formulations include, but are not limited to, organic acid salts such as sodium, potassium, ammonium, or trihydroxyethylamino salts of citric acid, ascorbic acid, gluconic acid, carbonic acid, tartaric acid, succinic acid, acetic acid or phtalic acid; Tris, tromethamine hydrochloride, sulfate or phosphate buffer. In addition, amino acid cationic components can also be used as buffering agent. Such amino acid component includes without limitation arginine, glycine, glycylglycine, and histidine. The arginine buffers include arginine acetate, arginine chloride, arginine phosphate, arginine sulfate, arginine succinate, etc. In one embodiment, the arginine buffer is arginine acetate. Examples of histidine buffers include histidine chloride-arginine chloride, histidine acetate-arginine acetate, histidine phosphate-arginine phosphate, histidine sulfate-arginine sulfate, histidine succinate-argine succinate, etc. The formulations of the buffers have a pH of 4.5 to pH 7.5, preferably from about 4.5 to about 6.5, more preferably from about 5.0 to about 6.2. In some embodiments, the concentration of the organic acid salts in the buffer is from about 10 mM to about 500 mM.

A “polyol” that may optionally be included in the formulation is a substance with multiple hydroxyl groups. Polyols can be used as stabilizing excipients and/or isotonicity agents in both liquid and lyophilized formulations. Polyols can protect biopharmaceuticals from both physical and chemical degradation pathways. Preferentially excluded co-solvents increase the effective surface tension of solvent at the protein interface whereby the most energetically favorable structural conformations are those with the smallest surface areas. Polyols include sugars (reducing and nonreducing sugars), sugar alcohols and sugar acids. A “reducing sugar” is one which contains a hemiacetal group that can reduce metal ions or react covalently with lysine and other amino groups in proteins and a “nonreducing sugar” is one which does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Sugar alcohols are selected from mannitol, xylitol, erythritol, maltitol, lactitol, erythritol, threitol, sorbitol and glycerol. Sugar acids include L-gluconate and metallic salts thereof. The polyol in the liquid formula or in the formulated lyophilized solid can be 0.0%-20% by weight. Preferably, a nonreducing sugar, sucrose or trehalose at a concentration of about from 0.1% to 15% is chosen in the formulation, wherein trehalose being preferred over sucrose, because of the solution stability of trehalose.

A surfactant optionally in the formulations is selected from polysorbate (polysorbate 20, polysorbate 40, polysorbate 65, polysorbate 80, polysorbate 81, polysorbate 85 and the like); poloxamer (e.g. poloxamer 188, poly(ethylene oxide)-poly(propylene oxide), poloxamer 407 or polyethylene-polypropylene glycol and the like); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamido-propyl-betaine (e.g. lauroamidopropyl); myristamidopropyl-, palmidopropyl-, or isostearamido-propyl-dimethylamine; sodium methyl cocoyl-, or disodium methyl oleyl-taurate; dodecyl betaine, dodecyl dimethylamine oxide, cocamidopropyl betaine and coco ampho glycinate; and the MONAQUAT™ series (e.g. isostearyl ethylimidonium ethosulfate); polyethyl glycol, polypropyl glycol, and copolymers of ethylene and propylene glycol (e.g. Pluronics, PF68 etc); etc. Preferred surfactants are polyoxyethylene sorbitan fatty acid esters e.g. polysorbate 20, 40, 60 or 80 (Tween 20, 40, 60 or 80). The concentration of a surfactant in the formulation is range from 0.0% to about 2.0% by weight. In certain embodiments, the surfactant concentration is from about 0.01% to about 0.2%. In one embodiment, the surfactant concentration is about 0.02%.

A “preservative” optionally in the formulations is a compound that essentially reduces bacterial action therein. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The preservative in the liquid formula or in the formulated lyophilized powder can be 0.0%-5.0% by weight. In one embodiment, the preservative herein is benzyl alcohol.

Suitable free amino acids as a bulky material, or tonicity agent, or osmotic pressure adjustment in the formulation, is selected from, but are not limited to, one or more of arginine, cystine, glycine, lysine, histidine, ornithine, isoleucine, leucine, alanine, glycine glutamic acid or aspartic acid. The inclusion of a basic amino acid is preferred i.e. arginine, lysine and/or histidine. If a composition includes histidine then this may act both as a buffering agent and a free amino acid, but when a histidine buffer is used it is typical to include a non-histidine free amino acid e.g. to include histidine buffer and lysine. An amino acid may be present in its D- and/or L-form, but the L-form is typical. The amino acid may be present as any suitable salt e.g. a hydrochloride salt, such as arginine-HCl. The amino acid in the liquid formula or in the formulated lyophilized powder can be 0.0%-30% by weight.

The formulations can optionally comprise methionine, glutathione, cysteine, cystine or ascorbic acid as an antioxidant at a concentration of about up to 5 mg/ml in the liquid formula or 0.0%-5.0% by weight in the formulated lyophilized powder; The formulations can optionally comprise metal chelating agent, e.g., EDTA, EGTA, etc., at a concentration of about up to 2 mM in the liquid formula or 0.0%-0.3% by weight in the formulated lyophilized powder.

The final formulation can be adjusted to the preferred pH with a buffer adjusting agent (e.g. an acid, such as HCl, H₂SO₄, acetic acid, H₃PO₄, citric acid, etc, or a base, such as NaOH, KOH, NH₄OH, ethanolamine, diethanolamine or triethanol amine, sodium phosphate, potassium phosphate, trisodium citrate, tromethamine, etc) and the formulation should be controlled “isotonic” which is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example. The isotonic agent is selected from mannitol, sorbitol, sodium acetate, potassium chloride, sodium phosphate, potassium phosphate, trisodium citrate, or NaCl. In general, both the buffer salts and the isotonic agent may take up to 30% by weight in the formulation.

Other excipients which may be useful in either a liquid or lyophilized formulation of the patent application include, for example, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluoronic F-127, cellulose, cyclodextrin, (2-Hydroxypropyl)-β-cyclodextrin, dextran (10, 40 and/or 70 kD), polydextrose, maltodextrin, ficoll, gelatin, hydroxypropylmeth, sodium phosphate, potassium phosphate, ZnCl₂, zinc, zinc oxide, sodium citrate, trisodium citrate, tromethamine, copper, fibronectin, heparin, human serum albumin, protamine, glycerin, glycerol, EDTA, metacresol, benzyl alcohol, phenol, polyhydric alcohols, or polyalcohols, hydrogenated forms of carbohydrate having a carbonyl group reduced to a primary or secondary hydroxyl group.

Other contemplated excipients, which may be utilized in the aqueous pharmaceutical compositions of the patent application include, for example, flavoring agents, antimicrobial agents, sweeteners, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4^{th} edition, Rowe et al., Eds., American Pharmaceuticals Association (2003); and Remington: the Science and Practice of Pharmacy, 21^{th} edition, Gennaro, Ed., Lippincott Williams & Wilkins (2005).

A pharmaceutical container or vessel is used to hold the pharmaceutical formulation of any of conjugates of the patent application. The vessel is a vial, bottle, pre-filled syringe, pre-filled or auto-injector syringe. The liquid formula can be freeze-dried or drum-dryed to a form of cake or powder in a borosilicate vial or soda lime glass vial. The solid powder can also be prepared by efficient spray drying, and then packed to a vial or a pharmaceutical container for storage and distribution.

In a further embodiment, the invention provides a method for preparing a formulation comprising the steps of: (a) lyophilizing the formulation comprising the conjugates, excipients, and a buffer system; and (b) reconstituting the lyophilized mixture of step (a) in a reconstitution medium such that the reconstituted formulation is stable. The formulation of step (a) may further comprise a stabilizer and one or more excipients selected from a group comprising bulking agent, salt, surfactant and preservative as hereinabove described. As reconstitution media, several diluted organic acids or water, i.e. sterile water, bacteriostatic water for injection (BWFI) or may be used. The reconstitution medium may be selected from water, i.e. sterile water, bacteriostatic water for injection (BWFI) or the group consisting of acetic acid, propionic acid, succinic acid, sodium chloride, magnesium chloride, acidic solution of sodium chloride, acidic solution of magnesium chloride and acidic solution of arginine, in an amount from about 10 to about 250 mM.

A liquid pharmaceutical formulation of the conjugates of the patent application should exhibit a variety of pre-defined characteristics. One of the major concerns in liquid drug products is stability, as proteins/antibodies tend to form soluble and insoluble aggregates during manufacturing and storage. In addition, various chemical reactions can occur in solution (deamidation, oxidation, clipping, isomerization etc.) leading to an increase in degradation product levels and/or loss of bioactivity. Preferably, a conjugate in either liquid or loyphilizate formulation should exhibit a shelf life of more than 6 months at 25° C. More preferred a conjugate in either liquid or loyphilizate formulation should exhibit a shelf life of more than 12 months at 25° C. Most preferred liquid formulation should exhibit a shelf life of about 24 to 36 months at 2-8° C. and the loyphilizate formulation should exhibit a shelf life of about preferably up to 60 months at 2-8° C. Both liquid and loyphilizate formulations should exhibit a shelf life for at least two years at −20° C., or −70° C.

In certain embodiments, the formulation is stable following freezing (e. g., −20° C., or −70° C.) and thawing of the formulation, for example following 1, 2 or 3 cycles of freezing and thawing. Stability can be evaluated qualitatively and/or quantitatively in a variety of different ways, including evaluation of drug/antibody(protein) ratio and aggregate formation (for example using UV, size exclusion chromatography, by measuring turbidity, and/or by visual inspection); by assessing charge heterogeneity using cation exchange chromatography, image capillary isoelectric focusing (icIEF) or capillary zone electrophoresis; amino-terminal or carboxy-terminal sequence analysis; mass spectrometric analysis, or matrix-assisted laser desorption ionization/time-of-flight mass spectrometry (MALDI/TOF MS), or HPLC-MS/MS; SDS-PAGE analysis to compare reduced and intact antibody; peptide map (for example tryptic or LYS-C) analysis; evaluating biological activity or antigen binding function of the antibody; etc. Instability may involve any one or more of: aggregation, deamidation (e.g. Asn deamidation), oxidation (e.g. Met oxidation), isomerization (e.g. Asp isomeriation), clipping/hydrolysis/fragmentation (e.g. hinge region fragmentation), succinimide formation, unpaired cysteine(s), N-terminal extension, C-terminal processing, glycosylation differences, etc.

A stable conjugate should also “retains its biological activity” in a pharmaceutical formulation, if the biological activity of the conjugate at a given time, e. g. 12 month, within about 20%, preferably about 10% (within the errors of the assay) of the biological activity exhibited at the time the pharmaceutical formulation was prepared as determined in an antigen binding assay, and/or in vitro, cytotoxic assay, for example.

For clinical in vivo use, the conjugate via the linkers of the invention will be supplied as solutions or as a lyophilized solid that can be redissolved in sterile water for injection. Examples of suitable protocols of conjugate administration are as follows. Conjugates are given daily, weekly, biweekly, triweekly, once every four weeks or monthly for 8˜54 weeks as an i.v. bolus. Bolus doses are given in 50 to 1000 ml of normal saline to which human serum albumin (e.g. 0.5 to 1 mL of a concentrated solution of human serum albumin, 100 mg/mL) can optionally be added. Dosages will be about 50 μg to 20 mg/kg of body weight per week, i.v. (range of 10 μg to 200 mg/kg per injection). 4˜54 weeks after treatment, the patient may receive a second course of treatment. Specific clinical protocols with regard to route of administration, excipients, diluents, dosages, times, etc., can be determined by the skilled clinicians.

Examples of medical conditions that can be treated according to the in vivo or ex vivo methods of killing selected cell populations include malignancy of any types of cancer, autoimmune diseases, graft rejections, and infections (viral, bacterial or parasite).

The amount of a conjugate which is required to achieve the desired biological effect, will vary depending upon a number of factors, including the chemical characteristics, the potency, and the bioavailability of the conjugates, the type of disease, the species to which the patient belongs, the diseased state of the patient, the route of administration, all factors which dictate the required dose amounts, delivery and regimen to be administered.

In general terms, the conjugates via the linkers of this invention may be provided in an aqueous physiological buffer solution containing 0.1 to 10% w/v conjugates for parenteral administration. Typical dose ranges are from 1 μg/kg to 0.1 g/kg of body weight daily; weekly, biweekly, triweekly, or monthly, a preferred dose range is from 0.01 mg/kg to 20 mg/kg of body weight weekly, biweekly, triweekly, or monthly, an equivalent dose in a human. The preferred dosage of drug to be administered is likely to depend on such variables as the type and extent of progression of the disease or disorder, the overall health status of the particular patient, the relative biological efficacy of the compound selected, the formulation of the compound, the route of administration (intravenous, intramuscular, or other), the pharmacokinetic properties of the conjugates by the chosen delivery route, and the speed (bolus or continuous infusion) and schedule of administrations (number of repetitions in a given period of time).

The conjugates via the linkers of the present invention are also capable of being administered in unit dose forms, wherein the term “unit dose” means a single dose which is capable of being administered to a patient, and which can be readily handled and packaged, remaining as a physically and chemically stable unit dose comprising either the active conjugate itself, or as a pharmaceutically acceptable composition, as described hereinafter. As such, typical total daily/weekly/biweekly/monthly dose ranges are from 0.01 to 100 mg/kg of body weight. By way of general guidance, unit doses for humans range from 1 mg to 3000 mg per day, or per week, per two weeks (biweekly), triweekly, or per month. Preferrably the unit dose range is from 1 to 500 mg administered one to four times a month and even more preferably from 1 mg to 100 mg, once a week, or once a biweek, or once a triweek. Conjugates provided herein can be formulated into pharmaceutical compositions by admixture with one or more pharmaceutically acceptable excipients. Such unit dose compositions may be prepared for use by oral administration, particularly in the form of tablets, simple capsules or soft gel capsules; or intranasal, particularly in the form of powders, nasal drops, or aerosols; or dermally, for example, topically in ointments, creams, lotions, gels or sprays, or via transdermal patches.

In yet another embodiment, a pharmaceutical composition comprising a therapeutically effective amount of the conjugate of Formula (I), (II) or (III) or any conjugates described through the present patent can be administered concurrently with the other therapeutic agents such as the chemotherapeutic agent, the radiation therapy, immunotherapy agents, autoimmune disorder agents, anti-infectious agents or the other conjugates for synergistically effective treatment or prevention of a cancer, or an autoimmune disease, or an infectious disease. The synergistic agents are preferably selected from one or several of the following drugs: Abatacept, Abiraterone acetate, Abraxane, Acetaminophen/hydrocodone, Acalabrutinib, aducanumab, Adalimumab, ADXS31-142, ADXS-HER2, afatinib dimaleate, aldesleukin, alectinib, alemtuzumab, Alitretinoin, ado-trastuzumab emtansine, Amphetamine/dextroamphetamine, anastrozole, Aripiprazole, anthracyclines, Aripiprazole, Atazanavir, Atezolizumab, Atorvastatin, Avelumab, Axicabtagene ciloleucel, axitinib, belinostat, BCG Live, Bevacizumab, bexarotene, blinatumomab, Bortezomib, bosutinib, brentuximab vedotin, brigatinib, Budesonide, Budesonide/formoterol, Buprenorphine, Cabazitaxel, Cabozantinib, capmatinib, Capecitabine, carfilzomib, chimeric antigen receptor-engineered T (CAR-T) cells, Celecoxib, ceritinib, Cetuximab, Chidamide, Ciclosporin, Cinacalcet, crizotinib, Cobimetinib, Cosentyx, crizotinib, CTL019, Dabigatran, dabrafenib, dacarbazine, daclizumab, dacomotinib, daptomycin, Daratumumab, Darbepoetin alfa, Darunavir, dasatinib, denileukin diftitox, Denosumab, Depakote, Dexlansoprazole, Dexmethylphenidate, Dexamethasone, DigniCap Cooling System, Dinutuximab, Doxycycline, Duloxetine, Duvelisib, durvalumab, elotuzumab, Emtricitabine/Rilpivirine/Tenofovir, disoproxil fumarate, Emtricitbine/tenofovir/efavirenz, Enoxaparin, ensartinib, Enzalutamide, Epoetin alfa, erlotinib, Esomeprazole, Eszopiclone, Etanercept, Everolimus, exemestane, everolimus, exenatide ER, Ezetimibe, Ezetimibe/simvastatin, Fenofibrate, Filgrastim, fingolimod, Fluticasone propionate, Fluticasone/salmeterol, fulvestrant, gazyva, gefitinib, Glatiramer, Goserelin acetate, Icotinib, Imatinib, Ibritumomab tiuxetan, ibrutinib, idelalisib, ifosfamide, Infliximab, imiquimod, ImmuCyst, Immuno BCG, iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144, lorlatinib, Memantine, Methylphenidate, Metoprolol, Mekinist, mericitabine/Rilpivirine/Tenofovir, Modafinil, Mometasone, Mycidac-C, Necitumumab, neratinib, Nilotinib, niraparib, Nivolumab, ofatumumab, obinutuzumab, olaparib, Olmesartan, Olmesartan/hydrochlorothiazide, Omalizumab, Omega-3 fatty acid ethyl esters, Oncorine, Oseltamivir, Osimertinib, Oxycodone, palbociclib, Palivizumab, panitumumab, panobinostat, pazopanib, pembrolizumab, PD-1 antibody, PD-L1 antibody, Pemetrexed, pertuzumab, Pneumococcal conjugate vaccine, pomalidomide, Pregabalin, ProscaVax, Propranolol, Quetiapine, Rabeprazole, radium 223 chloride, Raloxifene, Raltegravir, ramucirumab, Ranibizumab, regorafenib, Rituximab, Rivaroxaban, romidepsin, Rosuvastatin, ruxolitinib phosphate, Salbutamol, savolitinib, semaglutide, Sevelamer, Sildenafil, siltuximab, Sipuleucel-T, Sitagliptin, Sitagliptin/metformin, Solifenacin, solanezumab, Sonidegib, Sorafenib, Sunitinib, tacrolimus, tacrimus, Tadalafil, tamoxifen, Tafinlar, Talimogene laherparepvec, talazoparib, Telaprevir, talazoparib, Temozolomide, temsirolimus, Tenofovir/emtricitabine, tenofovir disoproxil fumarate, Testosterone gel, Thalidomide, TICE BCG, Tiotropium bromide, Tisagenlecleucel, toremifene, trametinib, Trastuzumab, Trabectedin (ecteinascidin 743), trametinib, tremelimumab, Trifluridine/tipiracil, Tretinoin, Uro-BCG, Ustekinumab, Valsartan, veliparib, vandetanib, vemurafenib, venetoclax, vorinostat, ziv-aflibercept, Zostavax, and their analogs, derivatives, pharmaceutically acceptable salts, carriers, diluents, or excipients thereof, or a combination above thereof.

The drugs/cytotoxic agents used for conjugation via a branched linker of the present patent can be any analogues and/or derivatives of amatoxin described in the present patent. One skilled in the art of drugs/cytotoxic agents will readily understand that each of the amatoxin described herein can be modified in such a manner that the resulting compound still retains the specificity and/or activity of the starting compound. The skilled artisan will also understand that many of these analogs or derivative compounds can be used in place of the drug analogs described herein. Thus, the drug analogs of the present invention include many analogues and derivatives that may not be described in detail thereof.

All references cited herein and in the examples that follow are expressly incorporated by reference in their entireties.

### EXAMPLES

The invention is further described in the following examples, which are not intended to limit the scope of the invention. Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ), or The Shanghai Cell Culture Institute of Chinese Academy of Science, unless otherwise specified. Cell culture reagents were obtained from Invitrogen Corp., unless otherwise specified. All anhydrous solvents were commercially obtained and stored in Sure-Seal bottles under nitrogen. PEG compounds were purchased from Biomatrik Inc, Jiaxing, China. Topotecan, Maytansinol, MMAE, MMAF, Exatecan, Eribulin, and their derivatives or major components were bought from several commercial sources, such as from Chengdu Tianyuan Natural Product Co., Ltd, Chengdu, China; Brightgene Biomedical Co., Suzhou, China; etc. Experimental animals were purchased from National Resource Center of Model Mice via (iemiPharmnatech.Co., Ltd, Najing, China and Shanghai SLAC Laboratory Animal Co., Ltd., Shanghai, China; T-DM1 was purchased from Roche via a pharmacy in Hong Kong, China. All other reagents and solvents were purchased as the highest grade available and used without further purification. The preparative HPLC separations were performed with Varain PreStar HPLC. NMR spectra were recorded on Bruker 500 MHz Instrument. Chemical shifts (delta) are reported in parts per million (ppm) referenced to tetramethylsilane at 0.00 and coupling constants (J) are reported in Hz. The mass spectral data were acquired on a Waters Xevo G2 QTOF mass spectrum equipped with Waters Acquity UPLC separations module and Acquity TUV detector. In general, the UPLC separation was run on C₈ column with mobile phase A, 1% formic acid and phase B, 100% CH₃CN.

### Example 1. Synthesis of tert-butyl 2,5,8,11,14,17,20,23,26-nonaoxaoctacosan-28-oate (1)

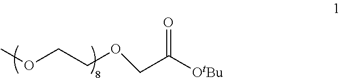

NaH (60%, 8.0 g, 200 mmol) was added to a solution of mPEG₈-OH (38.4 g, 100 mmol) in THF (1.0 L). After stirring at r.t. for 30 min, tert-butyl 2-bromoacetate (48.8 g, 250 mmol) was added to the mixture, and stirred at r.t. for 1 h. The mixture was then poured onto ice water, extracted with DCM, and the organic layer was washed with brine, dried over anhydrous sodium sulfate. Purification by column chromatography (0% to 5% methanol/dichloromethane) yielded compound 1 as a yellow oil (27.6 g, 59% yield). ESI MS m/z 499.40 ([M+H]⁺).

### Example 2. Synthesis of 2,5,8,11,14,17,20,23,26-nonaoxaoctacosan-28-oic acid (2)

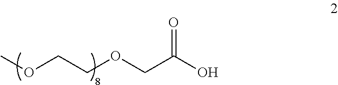

Compound 1 (29.4 g, 59.0 mmol) was dissolved in DCM (400 mL), and then formic acid (600 mL) was added. The resulting solution was stirred at 25° C. overnight. All volatiles were removed under vacuum, which afforded the title product as a yellow oil (26.1 g, >100% yield). ESI m/z calcd. for C₁₉H₃₉O₁₁ [M+H]⁺: 443.24, found 443.25.

### Example 3. Synthesis of Compound 3

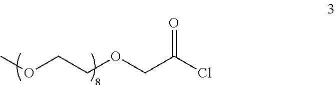

To the solution of compound 2 (59.0 mmol) dissolved in DCM (600 mL), (COCl)₂ (100 mL) and DMF (41 g, 0.59 mmol) were added. The resulting solution was stirred at r.t. for 4 h. All volatiles were removed under vacuum to yield the title product as a yellow oil. ESI MS m/z 461.38 ([M+H]⁺).

### Example 4. Synthesis of Compound 4

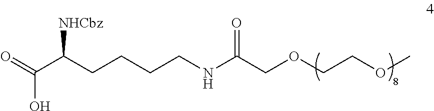

Z-L-Lys-OH (33.1 g, 118.0 mmol), Na₂CO₃ (18.7 g, 177.1 mmol) and NaOH (4.7 g, 118.0 mmol) were dissolved in water (700 mL). The mixture was cooled to 0° C., to which a solution of compound 3 (59.0 mmol) in THF (20 mL) was added. The resulting mixture was stirred at r.t. for 1 h. THF was removed under vacuum, and concentrated HCl was added to the aqueous solution until pH reached 3 under ice cooling. After extraction with DCM, the organic layer was washed with brine, dried over sodium sulfate and concentrated to give the title product as a yellow oil (44 g, 99% yield). ESI m/z calcd. for C₃₃H₅₇N₂O₁₄ [M+H]⁺: 705.40, found 705.39.

### Example 5. Synthesis of Compound 5

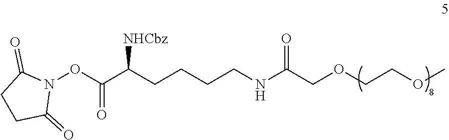

Compound 4 (20 g, 28.4 mmol, 1.0 eq) was dissolved in 350 mL of anhydrous DCM and cooled over ice water bath. NHS (3.9 g, 34.1 mmol, 1.2 eq) and EDC (27 g, 142.0 mmol, 5.0 eq) were added in sequence. The reaction was allowed to stir at r.t. overnight and then washed with water (200 mL×2), brine (200 mL×1), dried over anhydrous sodium sulfate, concentrated. The residue was dissolved in a small amount of DCM, and loaded onto a silica gel column and eluted with 2:49:49 to 4:48:48 MeOH/EA/DCM/. The product was obtained as a yellow oil (14.2 g, 62% yield). ESI m/z calcd. for C₃₇H₆₀N₃O₁₆ [M+H]⁺: 802.4, found: 802.4.

### Example 6. Synthesis of (2S,4R)-5-(3-amino-4-hydroxyphenyl)-4-((tert-butoxycarbonyl)-amino)-2-methylpentanoic acid (Compound 7)

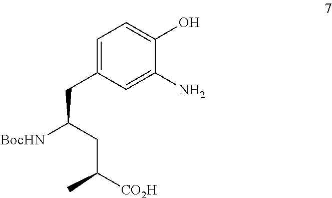 [SMILES: C[C@@H](C[C@H](Cc1ccc(O)c(N)c1)NC(=O)OC(C)(C)C)C(=O)O]

A mixture of (2S,4R)-4-((tert-butoxycarbonyl)amino)-5-(4-hydroxy-3-nitrophenyl)-2-methylpentanoic acid (Compound 6) (15 g, 0.041 mol, 1.0 eq) and palladium on carbon (2.0 g, 10 wt %) in 150 mL of methanol was stirred at r.t. under a H₂ balloon for 4 h. The catalyst was filtered off and washed with methanol. The filtrate was concentrated to give 13.8 g of crude material which was used directly in the next step (yield>100%). ESI m/z calcd. for C₁₇H₂₇N₂O₅ [M+H]⁺: 339.2, found: 339.2.

### Example 7. Synthesis of Compound 9

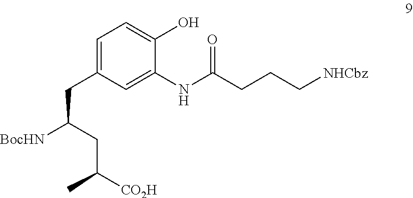 [SMILES: C[C@@H](C[C@H](Cc1ccc(O)c(NC(=O)CCCNC(=O)OCc2ccccc2)c1)NC(=O)OC(C)(C)C)C(=O)O]

To the crude product from the previous step (13.8 g, 0.041 mol, 1.0 eq) dissolved in 2 mL of ethanol and 0.2 mL of 0.1M NaH₂PO₄, 2,5-dioxopyrrolidin-1-yl 4-(((benzyloxy)-carbonyl)amino)-butanoate (15.0 g, 0.054 mol, 1.1 eq) was added. The reaction mixture was stirred overnight, concentrated and re-dissolved in DCM, dried over anhydrous sodium sulfate, filtered, concentrated, and purified by silica gel column (0-5% MeOH/DCM) to give a yellow oil (14.9 g, 66% yield). ESI m/z calcd. for C₂₉H₄₀N₃O₈ [M+H]⁺: 558.3, found: 558.3.

### Example 8. Synthesis of Compound 10

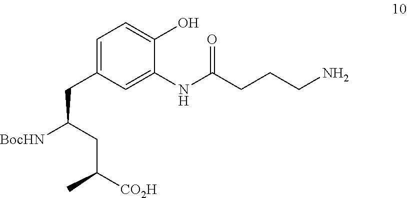 [SMILES: C[C@@H](C[C@H](Cc1ccc(O)c(NC(=O)CCCN)c1)NC(=O)OC(C)(C)C)C(=O)O]

Compound 9 (8.7 g, 15.08 mmol, 1.0 eq) and and palladium on carbon (1.0 g, 10 wt %) in 100 mL of methanol was stirred under a H₂ balloon at r.t. overnight. The catalyst was filtered off and washed with methanol. The filtrate was evaporated in vacuo to give 6.4 g of crude material which was used directly in the next step (yield>100%). ESI m/z calcd. for C₂₁H₃₄N₃O₆ [M+H]⁺: 424.2, found: 424.2.

### Example 9. Synthesis of Compound 11

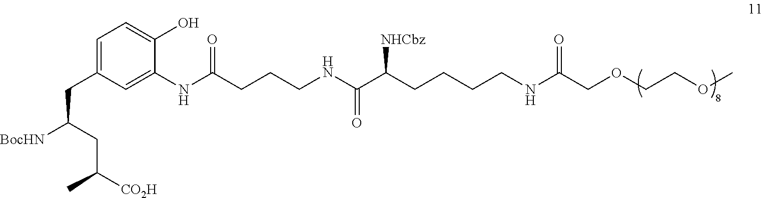

Compound 10 (6.4 g, 15.1 mmol, 1.0 eq) in the mixture of 40 mL of ethanol and 10 mL of 0.1M NaH₂PO₄ was added compound 5 (12.7 g, 15.9 mmol, 1.05 eq). The reaction mixture was stirred overnight, concentrated and dissolved in DCM, dried over anhydrous sodium sulfate, filtered, concentrated, and purified by silica gel column (3-5% MeOH/DCM) to give a white foam (11.7 g, 70% yield). ESI m/z calcd. for C₅₄H₈₈N₅O₁₉ [M+H]⁺: 1110.6, found: 1110.6.

### Example 10. Synthesis of Compound 12

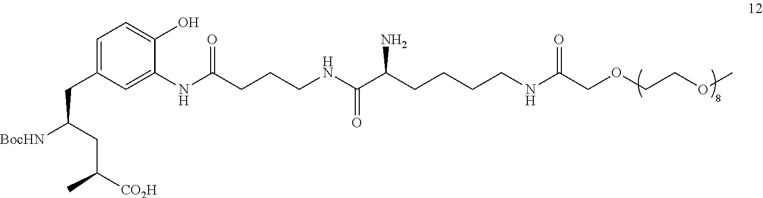

A mixture of compound 11 (4.2 g, 3.79 mmol, 1.0 eq) and palladium on carbon (0.4 g, 10 wt %) in 5 mL of methanol was stirred under H₂ balloon at r.t. overnight. The catalyst was filtered off and washed with methanol. The filtrate was evaporated to give 0.32 g of crude material which was used directly in the next step (yield 87%). ESI m/z calcd. for C₄₆H₈₂N₅O₁₇ [M+H]⁺: 1997.1, found: 1997.1.

### Example 11. Synthesis of meso-2,3-bis(benzylamino)succinic acid (Compound 13)

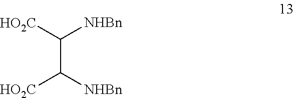

To a solution of meso-2,3-dibromosuccinic acid (50 g, 181 mmol) in EtOH (400 mL) was added benzylamine (150 mL) dropwise. After completion of addition, the mixture was heated to 90° C. and stirred overnight. The mixture was cooled to r.t. and diluted with water. 6 N HCl was added until pH 4 was reached, to give white precipitates. The precipitates were filtered, rinsed with water and dried to give meso-2,3-bis(benzylamino)succinic acid (50 g, 152 mmol, 84%).

### Example 12. Synthesis of meso-2,3-diaminosuccinic acid

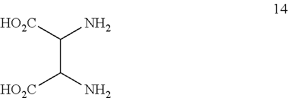

To a solution of meso-2,3-bis(benzylamino)succinic acid (18 g, 55 mmol) in AcOH (100 mL) and HCl (100 mL) was added Pd/C (3 g, 10 wt %), and the mixture was stirred under 1 atm hydrogen atmosphere at 50° C. for 48 h. The catalyst was removed by filtration and washed with water. The filtrate was concentrated and the residue was dissolved in 1N NaOH (200 mL). Acetic acid was added until pH 5 was reached, to give white precipitates. The precipitates were filtered, rinsed with water and dried to give meso-2,3-diaminosuccinic acid (8.7 g, >100%).

### Example 13. Synthesis of meso-2,3-bis(((benzyloxy)carbonyl)amino)succinic acid

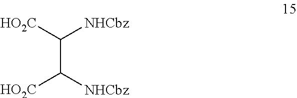 [SMILES: O=C(NC(C(=O)O)C(NC(=O)OCc1ccccc1)C(=O)O)OCc1ccccc1]

To a solution of meso-2,3-diaminosuccinic acid (31.74 g, 214 mmol) in THF (220 mL) and 4 N NaOH (214 mL) was added benzyl chloroformate (61 mL, 428 mmol) dropwise at 0° C. After completion of the addition, the mixture was allowed to warm to r.t. and stirred for 2 h. The reaction was diluted with water (1600 mL) and washed with ethyl acetate (2×1500 mL). The aqueous layer was separated and acidified with con.HCl until pH 2 was reached. The resultant solution was stirred for 1 h and stood at 5° C. to give white precipitates. The precipitates were filtered, rinsed with water and dried to give meso-2,3-bis(((benzyloxy)carbonyl)amino)succinic acid (52.2 g, 125 mmol, 59%).

### Example 14. Synthesis of dibenzyl ((3R,4S)-2,5-dioxotetrahydrofuran-3,4-diyl)dicarbamate

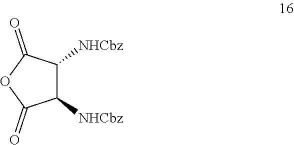 [SMILES: O=C(N[C@H]1C(=O)OC(=O)[C@@H]1NC(=O)OCc1ccccc1)OCc1ccccc1]

The solution of meso-2,3-bis(((benzyloxy)carbonyl)amino)succinic acid (5.0 g, 12 mmol) in Ac₂O (37.5 mL) was refluxed for 20 min, cooled and concentrated to give an anhydride. The diastereomeric mixture was treat with CHCl₃ (37 mL), the insoluble meso-isomer was filtered and the filtrate was treated with petroleum ether to give crystals of dibenzyl ((3R,4S)-2,5-dioxotetrahydrofuran-3,4-diyl)dicarbamate (racemic mixture, 2.0 g, 5 mmol, 42%).

### Example 15. Synthesis of Compound 17

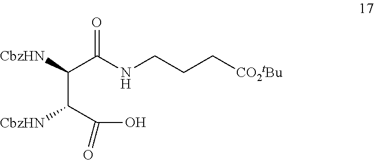 [SMILES: CC(C)(C)OC(=O)CCCNC(=O)[C@H](NC(=O)OCc1ccccc1)[C@@H](NC(=O)OCc1ccccc1)C(=O)O]

To a mixture of compound 16 (4.25 g, 10.68 mmol, 1.0 eq) and DMAP (13 mg, 0.11 mmol, 0.01 eq) in 20 mL of dry DCM, a solution of t-butyl aminobutyrate (1.78 g, 11.21 mmol, 1.05 eq) in 10 mL of anhydrous DCM was added. After the addition was completed, compound 16 was completely dissolved and the reaction was allowed to stir at r.t. overnight. The crude product was loaded on a silica gel column and eluted with 3-5% MeOH/DCM. Fractions were combined and evaporated, the residue was triturated with PE/DCM (1:1) to afford 3.3 g of a white solid (yield 55.9%). ESI m/z calcd. for C₂₈H₃₆N₃O₉ [M+H]⁺: 558.2, found: 558.2.

### Example 16. Synthesis of Compound 18

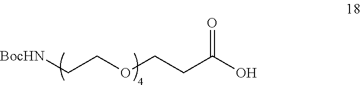

In a 500 mL flask, H₂N-PEG₄-CH₂CH₂CO₂H (3.0 g, 11.3 mmol, 1.0 eq) and K₂CO₃ (4.7 g, 33.93 mmol, 3.0 eq) were dissolved in 50 mL of water, and cooled over an ice water bath. Boc₂O (3.2 g, 14.7 mmol, 1.3) in 50 mL of THF was added dropwise. The reaction was allowed to warm to r.t. and stirred overnight. The reaction mixture was adjusted to pH 4-5 with 1N KHSO₄ and extracted with DCM (200 mL×1, 100 mL×3), washed with water (500 mL×1), and brine (500 mL×1), dried over anhydrous sodium sulfate, and concentrated. The residue was dissolved in a small amount of DCM and then loaded on a silica gel column, eluted with 24% MeOH/DCM, and the fractions were combined and concentrated to give 3.8 g of colorless oil (yield 93%). ESI m/z calcd. for C₁₆H₃₂NO₈ [M+H]⁺: 366.2, found: 366.2.

### Example 17. Synthesis of Compound 19

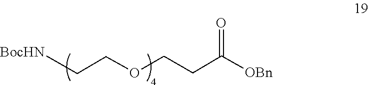

In a 50 mL single-necked flask, BocHN-PEG₄-CH₂CH₂CO₂H (0.81 g, 2.22 mmol, 1.0 eq), K₂CO₃ (0.92 g, 6.66 mmol, 3.0 eq) and NaI (0.033 g, 0.222 mmol, 0.1 eq) were mixed in 10 mL of DMF, cooled over an ice water bath, and BnBr (0.57 g, 3.33 mmol, 1.5 eq) was added dropwise, and the mixture was warmed to r.t. and stirred overnight. The reaction mixture was diluted with 100 mL of water, extracted with DCM (100 mL×2), washed with water (200 mL×1), and brine (200 mL×1), dry over anhydrous sodium sulfate, and concentrated. The residue was dissolved in a small amount of DCM, loaded on silica gel column, eluted with is 70-90% EA/PE to give 0.69 g of colorless oil (69% yield). ESI m/z calcd. for C₂₃H₃₈NO₈ [M+H]⁺: 446.3, found: 446.3.

### Example 18. Synthesis of Compound 20

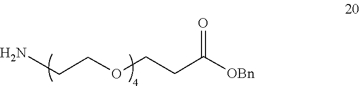

A solution of BocHN-PEG₄-CH₂CH₂CO₂Bn (0.69 g, 1.5 mmol, 1.0 eq) in 6 mL of DCM and 3 mL of TFA was stirred at r.t. for 30 min. The solvents were removed and the residue was co-evaporated with DCM for three times, placed on high vacuum pump. The crude product was used directly in the next reaction. ESI m/z calcd. for C₁₈H₃₀NO₆ [M+H]⁺: 356.2, found: 356.2.

### Example 19. Synthesis of Compound 21

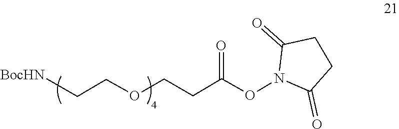

To a solution of BocHN-PEG₄-CH₂CH₂CO₂H (3.8 g, 10.4 mmol, 1.0 eq) in 50 mL of dry DCM, NHS (1.4 g, 12.5 mmol, 1.2 eq) and EDC (10.0 g, 52.0 mmol, 5.0 eq) were added. The reaction was stirred at r.t. overnight and then washed with water (50 mL×2), brine (100 mL×1), dried over anhydrous sodium sulfate, and concentrated. The crude product was used directly in the next step. ESI m/z calcd. for C₂₀H₃₅N₂O₁₀ [M+H]⁺: 463.2, found: 463.2.

### Example 20. Synthesis of Compound 22

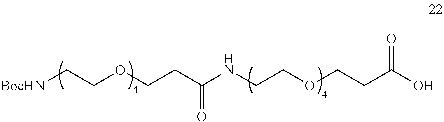

In a 300 mL flask, H₂N-PEG₄-CH₂CH₂CO₂H (2.8 g, 10.4 mmol, 1.0 eq) and K₂CO₃ (4.3 g, 31.2 mmol, 3.0 eq) were dissolved in 40 mL of water, cooled over an ice water bath, and the above crude NHS ester solution (3.8 g, 10.4 mmol, 1.0 eq) in 40 mL of THF was added dropwise, and the mixture was warmed to r.t. and stirred overnight. The reaction mixture was adjusted to pH 4-5 using 1N KHSO₄, extracted with DCM (150 mL×1, 100 mL×2), washed with water (200 mL×1), and brine (200 mL×1), dried over anhydrous sodium sulfate, and concentrated. The residue was dissolved in small amount of DCM, and the loaded on a silica gel column, eluted with 4-6% MeOH/DCM to give a colorless oil (5.18 g, 81% yield). ESI m/z calcd. for C₂₇H₅₃N₂O₁₃ [M+H]⁺: 613.3, found: 613.3.

### Example 21. Synthesis of Compound 23

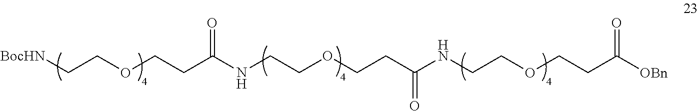

H₂N-PEG₄-CH₂CH₂CO₂Bn (crude product from the previous step) dissolved in 3 mL of DMF, cooled over ice/water bath, DIPEA (0.78 g, 6.0 mmol, 4.0 eq) was added dropwise, and followed by addition of a solution of compound 22 (0.93 g, 1.5 mmol, 1.0 eq) in 7 mL of DMF and HATU (1.72 g, 4.5 mmol, 3.0 eq). The reaction was stirred over the ice bath for 2 hours, and diluted with 100 mL of water, extracted with DCM (100 mL×3), washed with 1N KHSO₄ (200 mL×1), saturated sodium bicarbonate (200 mL×1), and brine (200 mL×1), dried over anhydrous sodium sulfate, and concentrated. The residue was dissolved in a small amount of DCM, loaded on a silica gel column, and eluted 0-5% MeOH/DCM. Fractions were combined and concentrated to give 1.0 g of light yellow oil (71% yield). ESI m/z calcd. for C₄₅H₈₀N₃O₁₈ [M+H]⁺: 950.5, found: 950.5.

### Example 22. Synthesis of Compound 24

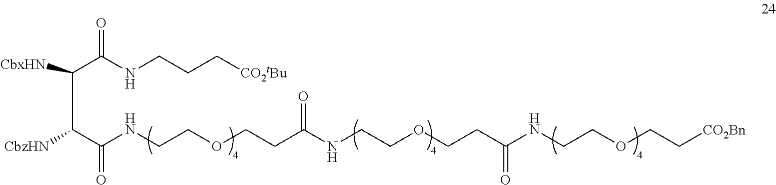

A solution of compound 23 (1.0 g, 1.03 mmol, 1.0 eq) in 6 mL of DCM, and 3 mL of TFA was stirred at r.t for 1 h. The solvents were removed and the residue was co-evaporated with DCM for three times, placed on high vacuum pump.

The crude product was re-dissolved in 10 mL of DMF, cooled over ice water bath. To which DIPEA (0.53 g, 4.12 mmol, 4.0 eq), compound 17 (0.56 g, 1.03 mmol, 1.0 eq) and HATU (1.17 g, 3.09 mmol, 3.0 eq) were added in sequence. After stirring over the ice bath for 1 hour, 100 mL of water was added, and a solid precipitated out. The solid was collected by filtration and washed with water, dissolved in DCM, dried over anhydrous sodium sulfate, filtered and concentrated. The residue was dissolved in a small amount of DCM, loaded on a silica gel column, and eluted 0-10% MeOH/DCM. Fractions were combined and concentrated to give 0.93 g of light yellow foam (yield 65%). ESI m/z calcd. for C₆₈H₁₀₇N₈O₂₆ [M+H]⁺: 1451.7, found: 1451.7.

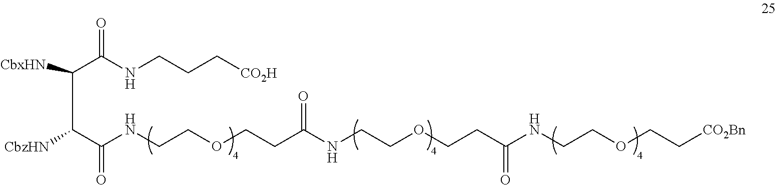

### Example 23. Synthesis of Compound 25

A solution of compound 24 (0.93 g, 0.67 mmol, 1.0 eq) in 6 mL of DCM, and 3 mL of TFA was stirred at r.t. for 1 h (the completion of the reaction was monitored by LC-MS). The solvents were removed and the residue was co-evaporated with DCM for three times, placed on high vacuum pump. The crude product was dissolved in a small amount of DCM and loaded on a silica gel column, and then eluted with 15-20% MeOH/DCM. Fractions were combined and concentrated to give 0.53 g of white foam (yield 60%) product. ESI m/z calcd. for C₆₄H₉₉N₈O₂₆ [M+H]⁺: 1395.7, found: 1395.7.

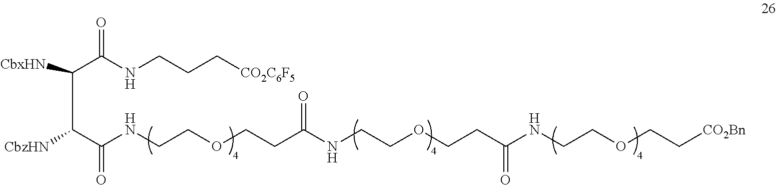

### Example 24. Synthesis of Compound 26

To compound 25 (0.53 g, 0.40 mmol, 1.0 eq) in 10 mL DCM, pentafluorophenol (0.081 g, 0.44 mmol, 1.1 eq) and EDC (0.38 g, 2.0 mmol, 5.0 eq) were added. The reaction mixture was stirred at r.t. overnight and then washed with cold water (5 mL×2) and brine (10 mL×1), dried over anhydrous sodium sulfate, filtered and concentrated. The crude product was used directly in the next step. ESI m/z calcd. for C₇₀H₉₈ F₅N₆O₂₆ [M+H]⁺: 1561.6, found: 1561.6.

### Example 25. Synthesis of Compound 27

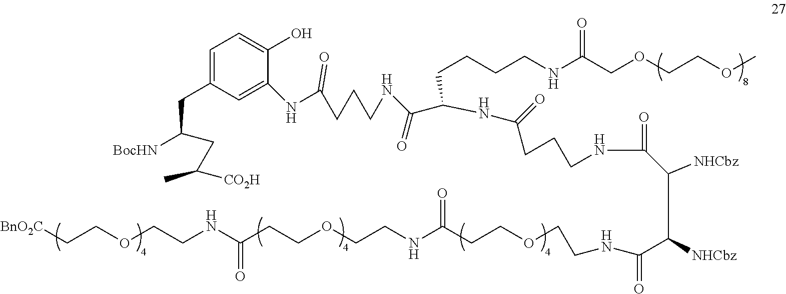

The crude product from the previous step (0.4 mmol, 1.0 eq) was dissolved in 10 mL DMF, cooled over ice water bath. To which compound 12 (0.39 g, 0.4 mmol, 1.0 eq) and DIPEA (0.15 g, 1.2 mmol, 3.0 eq) were added in sequence. After stirring over the ice bath for 1 hour, the reaction was concentrated, and re-dissolved in a small amount of DCM, loaded on a silica gel column and eluted with 0-20% MeOH/DCM to give a colorless oil (0.53 g, 58% yield). ESI m/z calcd. for C₁₁₀H₁₇₆N₁₁O₄₀ [M+H]⁺: 2291.2, found: 2291.2.

### Example 26. Synthesis of Compound 28

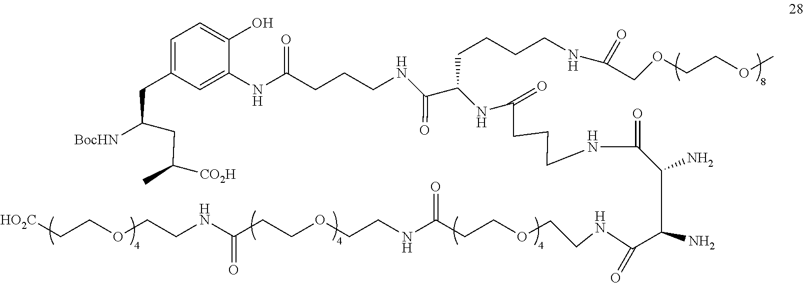

Compound 27 (0.53 g, 0.23 mmol, 1.0 eq) and dry palladium carbon (0.1 g, 10% wt) in 10 mL methanol was stirred under a H₂ balloon at r.t. overnight. The reaction mixture was filtered and the filtrate was evaporated to give 0.35 g of crude material, which was directly used for the next reaction (yield 80%). ESI m/z calcd. for C₈₇H₁₅₈N₁₁O₃ [M+H]⁺: 1933.1, found: 1933.1.

### Example 27. Synthesis of Compound 29

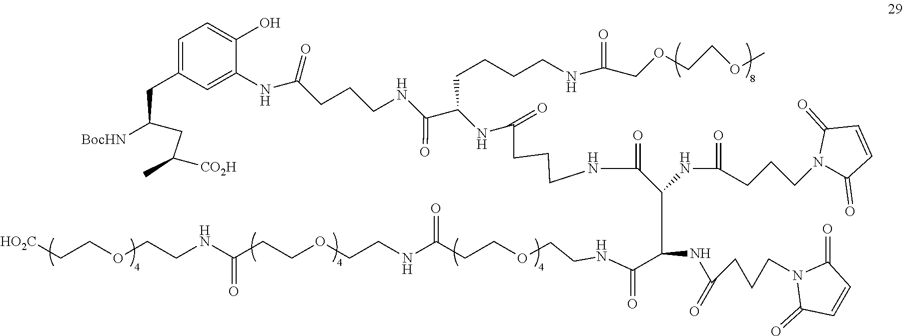

The crude product from the previous step (0.35 g, 0.18 mmol, 1.0 eq) re-dissolved in the mixture of 3 mL of ethanol, 0.2 mL of 0.1M NaH₂PO₄, N-(4-maleimidobutyryloxy) succinimide (0.20 g, 0.72 mmol, 4.0 eq) was added. The reaction mixture was stirred at r.t. overnight, and then concentrated and re-dissolved in DCM, dried over anhydrous sodium sulfate, filtered and concentrated. The residue was dissolved in a small amount of DCM, and loaded on silica gel column, eluted with 0-20% MeOH/DCM to give a colorless oil product (0.13 g, 33% yield). ESI m/z calcd. for C₁₀₃H₁₇₂N₁₃O₄₂ [M+H]⁺: 2263.2, found: 2263.2.

### Example 28. Synthesis of Compound 30

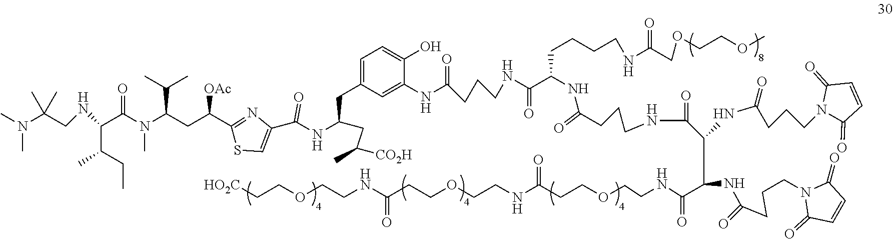

Compound 29 (0.13 g, 0.0574 mmol, 1.0 eq) was dissolved in 2 mL of DCM, and stirred with 2 mL of TFA at r.t. for 3 h. The solvents were removed and the residue was co-evaporated with DCM for three times, placed on high vacuum pump.

The crude product was re-dissolved in DMF and cooled over an ice water bath. Tub-pentafluorophenol (0.048 g, 0.0690 mmol, 1.0 eq) was added, followed by addition of DIPEA (0.022 g, 0.172 mmol, 3.0 eq). The reaction was stirred over the ice bath for 1 hour and then adjusted to pH 4-5 using formic acid. The mixture was concentrated, re-dissolved in a small amount of DCM, and loaded onto a silica gel column, and eluted with PE/EA and MeOH/DCM (all containing 0.10% formic acid). Fractions were combined and concentrated to give 0.1 g of yellow foam (70% yield). The product was further purified by preparative HPLC (45-50% MeCN/H₂O containing 0.1% formic acid). Fractions were combined and concentrated to give a colorless oil (0.030 g, 20% yield). ESI m/z calcd. for C₁₂₃H₂₀₄N₁₇O₄₅S [M+H]⁺: 2671.4, found: 2671.4.

### Example 29. Synthesis of Compound 31

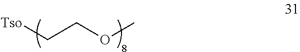

To a solution of mPEG₈-OH (10 g, 26 mmol, 1.0 eq) in 100 mL of anhydrous DCM, TEA (10.5 g, 104 mmol, 4.0 eq), DMAP (32 mg, 0.26 mmol, 0.01 eq) and TsCl (14.9 g, 78 mmol, 3.0 eq) were added in sequence over an ice water bath. The reaction was stirred at 0° C. for 10 min, then warmed to r.t. and stirred overnight. The reaction was washed with 1N HCl washing (100 mL×1), water (100 mL×1) and brine washing (100 mL×1), dried over anhydrous sodium sulfate, filtered and concentrated. The residue was dissolved in a small amount of DCM and loaded onto a silica gel column, eluted with EA/PE (5-100%) and 1-3% MeOH/DCM. Fractions were combined and concentrated to give a yellow oil (11.6 g, 83% yield). ESI m/z calcd. for C₂₄H₄₃O₁₁S [M+H]⁺: 539.2, found: 539.2.

### Example 30. Synthesis of Compound 32

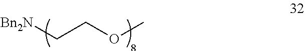

A mixture of compound 31 (11.6 g, 21.5 mmol, 1.0 eq) and dibenzylamine (5.5 g, 27.8 mmol, 1.5 eq) in 20 mL of anhydrous DMF was heated to 100° C. with stirring overnight. The reaction was diluted with 300 mL of DCM, washed with water (300 mL×3) and brine (300 mL×1), dried over anhydrous sodium sulfate, filtered and concentrated. The residue was purified on silica gel column (50-100% EA/PE) to give a light yellow oil (8.2 g, 66% yield). ESI m/z calcd. for C₃₁H₅₀NO₈ [M+H]⁺: 564.3, found: 564.3.

### Example 31. Synthesis of Compound 33

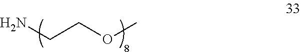

A solution mixture of compound 32 (8.6 g, 15.2 mmol, 1.0 eq) and dry palladium on carbon (0.9 g, 10 wt %) in 100 mL of anhydrous methanol was refluxed under a H₂ balloon overnight. The catalyst was filtered off and washed with methanol. The filtrate was evaporated to give 5.3 g of colorless oil (yield 90%). ESI m/z calcd. for C₁₇H₃₈NO₈ [M+H]⁺: 384.3, found: 384.3.

### Example 32. Synthesis of Compound 34

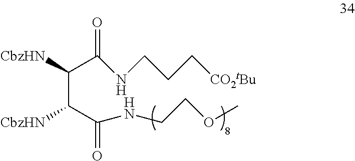

Compound 17 (1.6 g, 2.84 mmol, 1.0 eq) and compound 33 (1.2 g, 2.84 mmol, 1.0 eq) were dissolved in 5 mL of anhydrous DMF, to which HATU (3.2 g, 8.52 mmol, 3.0 eq) and DIPEA (1.5 g, 11.36 mmol, 4.0 eq) were added in sequence over an ice water bath. The reaction was stirred over the bath for 2 h, then 150 mL of water was added, and extracted with DCM (150 mL×1, 100 mL×1). The organic phase was washed with 1 N HCl (200 mL×1), saturated sodium bicarbonate (200 mL×1) and brine (200 mL×1), dried over anhydrous sodium sulfate, filtered and concentrated. The crude product was dissolved in a small amount of DCM and loaded on a silica gel column, and then eluted with 0-5% MeOH/DCM. Fractions were combined and concentrated to give 2.29 g of white solid (87% yield). ESI m/z calcd. for C₄₅H₇₁N₄O₁₆ [M+H]⁺: 923.5, found: 923.5.

### Example 33. Synthesis of Compound 35

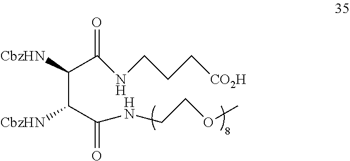

A solution of compound 34 (2.29 g, 2.48 mmol, 1.0 eq) in the mixture of 5 mL of DCM, and 5 mL of TFA was stirred at r.t. for 3 h. The solvents were removed and the residue was co-evaporated with DCM for three times, the residue was dissolved in a small amount of DCM, and loaded on a silica gel column, eluted with 5-8% MeOH/DCM. Fractions were combined and concentrated to give 2.09 g of white jelly solid (97% yield). ESI m/z calcd. for C₄₁H₆₃N₄O₁₆ [M+H]⁺: 867.4, found: 867.4.

### Example 34. Synthesis of Compound 36

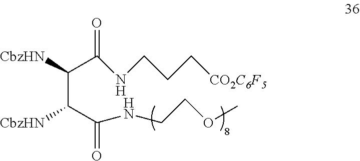

To compound 35 (1.5 g, 1.73 mmol, 1.0 eq) in 10 mL of DCM over an ice water bath, pentafluorophenol (0.35 g, 1.90 mmol, 1.1 eq) and EDC (1.7 g, 8.66 mmol, 5.0 eq) were added. The reaction was warmed to r.t. and stirred for 5 h, then washed with water (10 mL×2) and brine (20 mL×1), dried over anhydrous sodium sulfate, filtered and concentrated to give 1.07 g of crude product (60% yield). ESI m/z calcd. for C₄₇H₆₂ F₅N₄O₁₆ [M+H]⁺: 1033.4, found: 1033.4.

### Example 35. Synthesis of Compound 37

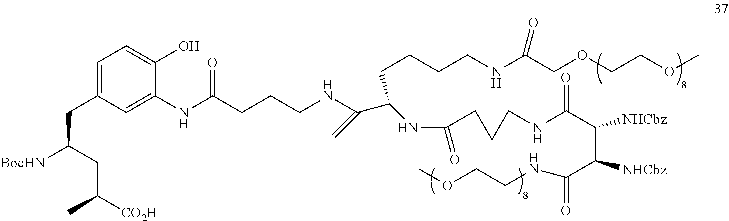

The above crude product (1.07 g, 1.0 mmol, 1.0 eq) in 10 mL of DMF over an ice water bath, compound 12 (0.92 g, 1.0 mmol, 1.0 eq) and DIPEA (0.39 g, 3.0 mmol, 3.0 eq) were added. The reaction was stirred over the bath for 1 h, and adjusted to pH 4-5 using 1N HCl, diluted with EA (100 mL), extracted with water (30 mL×5). The aqueous phase was concentrated and then re-dissolved in a small amount of DCM, loaded a silica gel column and eluted with 15-18% MeOH/DCM. Fractions were combined and concentrated to afford 0.88 g of colorless oil (51% yield). ESI m/z calcd. for C₈₇H₁₄₂N₉O₃₂ [M+H]⁺: 1825.0, found: 1825.0.

### Example 36. Synthesis of Compound 38

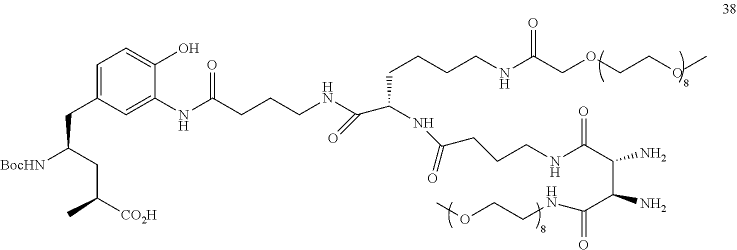

A mixture of compound 37 (0.88 g, 0.48 mmol, 1.0 eq) and palladium on carbon (0.1 g, 10 wt %) in 5 mL of methanol was stirred under a H₂ balloon at r.t. overnight. The catalyst was filtered and filtrated solution was concentrated to give 0.75 g of crude material, which was directly used for the next reaction (yield 80%). ESI m/z calcd. for C₇₁H₁₃₀N₉O₂₈ [M+H]⁺: 1556.9, found: 1556.9.

### Example 37. Synthesis of Compound 39

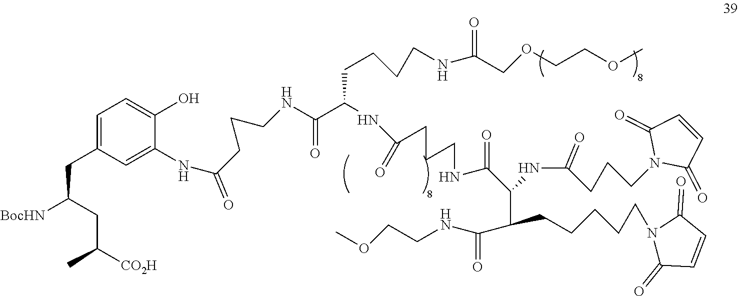

The crude product from the previous step (0.75 g, 0.48 mmol, 1.0 eq) dissolved in the mixture of 2 mL of ethanol and 0.2 mL of 0.1 M NaH₂PO₄, N-(4-maleimidobutyryloxy) succinimide (0.54 g, 1.92 mmol, 4.0 eq) was added. The reaction mixture was stirred at r.t. overnight, and then concentrated and re-dissolved in DCM, dried over anhydrous sodium sulfate, filtered and concentrated. The residue was dissolved in a small amount of DCM, and loaded on silica gel column, eluted with 0-20% MeOH/DCM to give a colorless oil (0.26 g, yield 29%). ESI m/z calcd. for C₈₇H₁₄₄N₁₁O₃₄ [M+H]⁺: 1887.0, found: 1887.0.

### Example 38. Synthesis of Compound 40

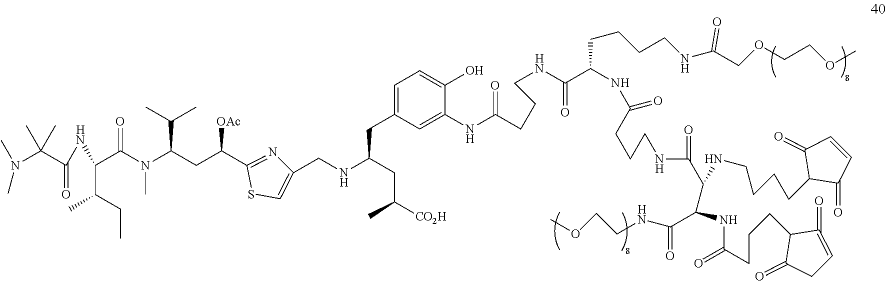

Compound 39 (0.26 g, 0.138 mmol, 1.0 eq) in 3 mL of DCM and 1 mL of TFA was stirred at r.t. for 1 h. The solvents were removed and the residue was co-evaporated with DCM for three times, placed on high vacuum pump.

The crude product was re-dissolved in 5 mL of DMF and cooled over an ice water bath. Tub-PFP (0.114 g, 0.166 mmol, 1.2 eq) and DIPEA (0.265 g, 2.07 mmol) were added in. The reaction was stirred over the ice bath for 1 hour and then adjusted to pH 4-5 using formic acid. The mixture was concentrated, re-dissolved in a small amount of DCM, and loaded onto a silica gel column, and eluted with PE/EA and MeOH/DCM (all containing 0.1% formic acid). Fractions were combined and concentrated to give 0.2 g of yellow foam product (63% yield). The product was further purified by preparative HPLC (45-50% MeCN/H2O containing 0.1% formic acid). Fractions were combined and concentrated to give a colorless oil product (0.10 g, 23% yield). ESI m/z calcd. for C₁₀₇H₁₇₆N₁₅O₃₇S [M+H]⁺: 2295.2, found: 2295.2.

### Example 39. Synthesis of Compound 41

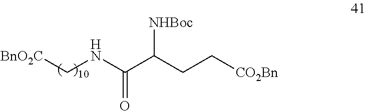

To a solution of benzyl 11-aminoundecanoate (2.91 g, 10.0 mmol) and Boc-Glu(OBzl)-OH (3.37 g, 10.0 mmol) in DMF (50 mL), EDC (1.91 g, 12.0 mmol) and TEA (3.5 mL, 25.0 mmol) were added. The reaction was stirred at r.t. for 8 h, diluted with water (100 ml) and extracted with EA (3×100 ml). The combined organic phases were washed once with 100 mL of brine, then dried over anhydrous sodium sulfate, filtered and concentrated. The residue was purified by SiO₂ column chromatography (EA/DCM, 1:15) to afford the title compound as a colorless oil (5.37 g, 88% yield).

### Example 40 Synthesis of Compound 42

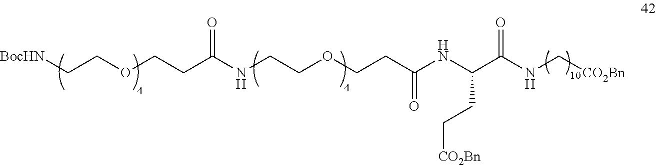

Compound 41 (0.64 g, 1.05 mmol, 1.0 eq) in the mixture of 5 mL of DCM and 2 mL of TFA was stirred at r.t. for 2 h, and then concentrated. The residue was co-evaporated with DCM for three times and placed under high vacuum pump. ESI m/z calcd. for C₃₀H₄₂N₂O₅ [M+H]⁺: 511.3, found: 511.3.

The above crude product was re-dissolved in 3 mL of DMF and cooled over an ice water bath. To which a solution of compound 22 (0.64 g, 1.05 mmol, 1.0 eq) in 7 mL of DMF was added, followed by addition of DIPEA (0.54 g, 4.20 mmol, 4.0 eq) and HATU (1.2 g, 3.15 mmol, 3.0 eq). The reaction was stirred over the bath for 1 h, then 100 mL of water was added, and extracted with DCM (150 mL×1, 100 mL×1). The organic phase was washed with 1 N KHSO₄ (200 mL×1), saturated sodium bicarbonate (200 mL×1) and brine (200 mL×1), dried over anhydrous sodium sulfate, filtered and concentrated. The crude product was dissolved in a small amount of DCM and loaded on a silica gel column, and then eluted with 0-10% MeOH/DCM. Fractions were combined and concentrated to give 0.94 g of light yellow oil (81% yield). ESI m/z calcd. for C₅₇H₉₂N₄O₁₇ [M+H]⁺: 1104.6, found: 1104.6.

### Example 41. Synthesis of Compound 43

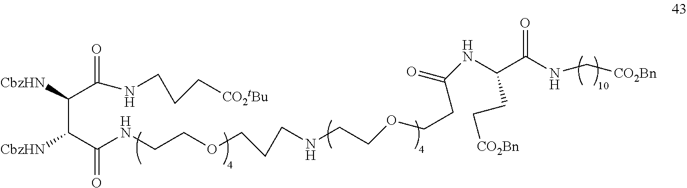

Compound 42 (0.94 g, 0.458 mmol, 1.0 eq) in the mixture of 7 mL of DCM, and 3 mL of TFA was stirred at r.t. for 2 h, and concentrated. The residue was co-evaporated with DCM for three times, then placed under high vacuum pump. ESI m/z calcd. for C₅₂H₈₄N₄O₁₅ [M+H]⁺: 1004.6, found: 1004.6.

The above crude compound was re-dissolved in 10 mL of DMF and cooled over an ice water bath, to which compound 17 (0.46 g, 0.85 mmol, 1.0 eq), DIPEA (0.44 g, 3.40 mmol, 4.0 eq) and HATU (0.97 g, 2.55 mmol, 3.0 eq) were added. The reaction was stirred over the bath for 1 h, then 100 mL of water was added, and a solid precipitated out. The solid was collected by filtrated and washed with water, dissolved in DCM and dried over anhydrous sodium sulfate, filtered and concentrated. The crude product was dissolved in a small amount of DCM and loaded on a silica gel column, and eluted with 0-10% MeOH/DCM. Fractions were combined and concentrated to give 1.13 g of light yellow oil product (87% yield). ESI m/z calcd. for C₈₀H₁₂₀N₉O₂₄ [M+H]⁺: 1590.8, found: 1590.8.

### Example 42. Synthesis of Compound 44

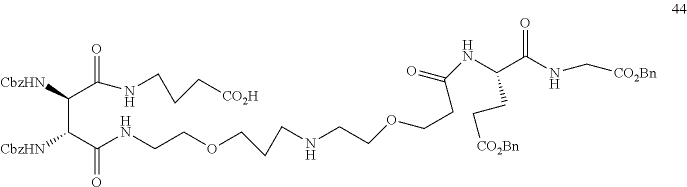 [SMILES: O=C(O)CCCNC(=O)[C@H](NC(=O)OCc1ccccc1)[C@@H](NC(=O)OCc1ccccc1)C(=O)NCCOCCCNCCOCCC(=O)N[C@@H](CCC(=O)OCc1ccccc1)C(=O)NCC(=O)OCc1ccccc1]

Compound 43 (1.13 g, 0.73 mmol, 1.0 eq) in the mixture of 7 mL of DCM, and 3 mL of TFA was stirred at r.t. for 3 h, and concentrated. The residue was co-evaporated with DCM for three times, dissolved in a small amount of DCM and loaded on a silica gel column, and eluted with 5-15% MeOH/DCM. Fractions were combined and concentrated to give 0.85 g of white foam product (78% yield). ESI m/z calcd. for C₇₆H₁₁₂N₉O₂₅ [M+H]⁺: 1550.8, found: 1550.8.

### Example 43. Synthesis of Compound 45

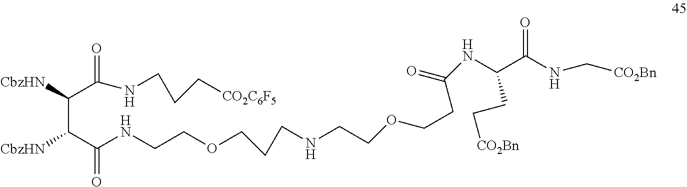

To compound 44 (0.85 g, 0.57 mmol, 1.0 eq) in 10 mL of DCM over an ice water bath, pentafluorophenol (0.11 g, 0.63 mmol, 1.1 eq) and EDC (0.55 g, 2.85 mmol, 5.0 eq) were added. The reaction mixture was warmed to r.t. and stirred overnight, then washed with ice water (10 mL×2) and cold brine (20 mL×1), dried over anhydrous sodium sulfate, filtered and concentrated. The crude product was used directly in the next step.

### Example 44. Synthesis of Compound 46

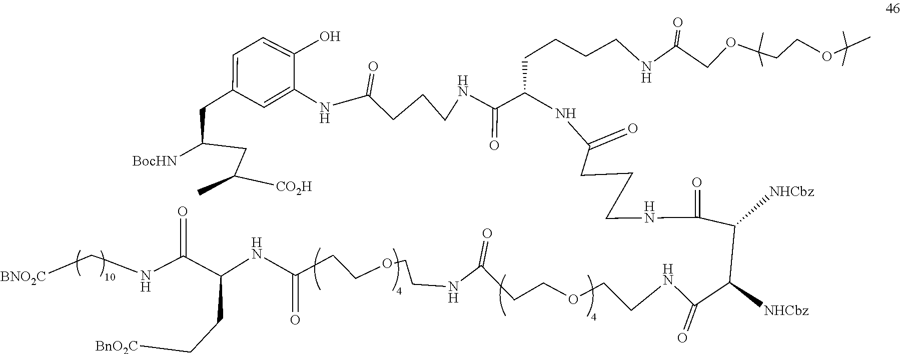

To the above crude product (0.94 g, 0.57 mmol, 1.0 eq) in 10 mL of DMF over an ice water bath, compound 12 (0.56 g, 0.57 mmol, 1.0 eq) and DIPEA (0.22 g, 1.71 mmol, 3.0 eq) were added. The reaction mixture was stirred over the bath for 3 h, and concentrated and then re-dissolved in a small amount of DCM, loaded a silica gel column and eluted with 12-20% MeOH/DCM. Fractions were combined and concentrated to afford 1.00 g of colorless oil (71% yield). ESI m/z calcd. for C₁₂₂H₁₈₉N₁₂O₃₉ [M+H]⁺: 2446.3, found: 2446.3.

### Example 45. Synthesis of Compound 47

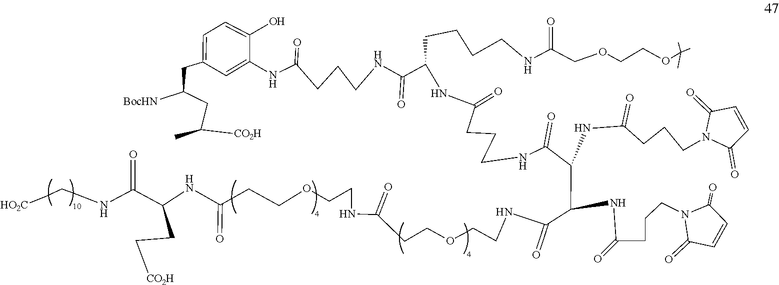

Compound 46 (0.53 g, 0.23 mmol, 1.0 eq) was dissolved in 5 mL of methanol, and dry palladium on carbon (0.1 g, 10 wt %) was added and the reaction was stirred under a H₂ balloon at r.t. overnight. The catalyst was filtered and filtrate was evaporated to give 0.71 g of crude material (yield 87%). ESI m/z calcd. for C₉₂H₁₆₅N₁₂O₃₅ [M+H]⁺: 1997.1, found: 1997.1.

The above crude product (0.71 g, 0.355 mmol, 1.0 eq) re-dissolved in 2 mL of ethanol and 0.2 mL of 0.1M NaH₂PO₄, N-(4-maleimidobutyryloxy) succinimide (0.40 g, 1.42 mmol, 4.0 eq) was added and the reaction mixture was stirred at r.t. overnight, concentrated and purified on prep C-18 HPLC column, eluted with 0-40% MeOH/H₂O to give a colorless oil (0.26 g, 33%). ESI m/z calcd. for C₁₀₈H₁₇₉N₁₄O₄₁ [M+H]⁺: 2328.2, found: 2328.2.

### Example 46. Synthesis of Compound 48

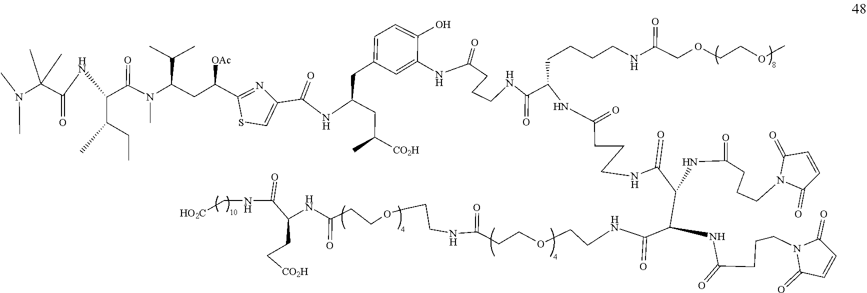

Compound 47 (0.26 g, 0.112 mmol, 1.0 eq) was stirred in the mixture of 2 mL of DCM and 2 mL of TFA at r.t. for 3 h. The solvents were removed and the residue was co-evaporated with DCM for three times, placed on high vacuum pump. ESI m/z calcd. for C₁₀₃H₁₇₁N₁₄O₃₉ [M+H]⁺: 2228.2, found: 2228.2.

The above crude product was re-dissolved in 5 mL of DMF and cooled over an ice water bath. Tub-PFP (0.0.93 g, 0.134 mmol, 1.2 eq) was added, followed by addition of DIPEA (0.043 g, 0.336 mmol, 3.0 eq). The reaction was stirred over the ice bath for 1 hour and then adjusted to pH 4-5 using formic acid. The mixture was concentrated, re-dissolved in a small amount of DCM, and loaded onto a silica gel column, and eluted with PE/EA and MeOH/DCM (containing 0.1% formic acid). Fractions were combined and concentrated to give 0.09 g of yellow foam. The product was dissolved in 50:50 MeOH/H₂O (2 mL) further purified by preparative HPLC (45-50% MeCN/H₂O containing 0.1% formic acid). Fractions were combined and concentrated to give a colorless oil (0.027 g, 15% yield). ESI m/z calcd. for C₁₂H₂₁₁N₁₈O₄₄S [M+H]⁺: 2736.4, found: 2736.4.

### Example 47. Synthesis of (S)-3,4-dimethyloxazolidine-2,5-dione (NCA)

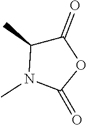

(S)-2-((tert-butoxycarbonyl)(methyl)amino)propanoic acid (5.0 g, 24.6 mmol) was dissolved in dichloromethane (95 mL) and allowed to stir in an ice/water bath for 15 minutes. After cooled triphosgene (8.7 g, 29.6 mmol, 1.2 eq) was added dropwise over approximately 5 minutes. After finish addition, the ice bath was removed, the reaction was allowed to proceed at room temperature for an addition 4 hrs. The solution was reduced under pressure and carbon tetrachloride (130 mL) was added to precipitate product. The white precipitate was collected by filtration and washed with the remaining carbon tetrachloride. After a few minutes the crystals were yellow and the yellow crystals were re-dissolved in a small amount of dichloromethane and the yellow gunk was removed by filtration. The filtrate (product) was then precipitated with carbon tetrachloride and filtrated. The crystals were allowed to dry on the filter for 3 hrs at ambient temperature to afford the title product (2.3 g, 69% yield). ESI MS, 130.05 (M+1)⁺.

### Example 48. Synthesis of May-NMA

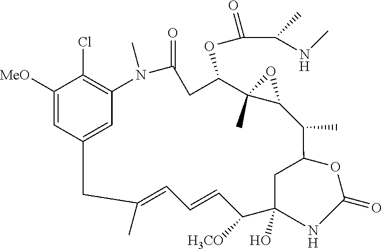 [SMILES: CN[C@@H](C)C(=O)O[C@H]1CC(=O)N(C)c2cc(cc(OC)c2Cl)C/C(C)=C/C=C/[C@@H](OC)[C@@]2(O)CC(OC(=O)N2)[C@@H](C)[C@H]2O[C@@]12C]

Maytansinol (200 mg, 0.354 mmol) was dissolved in DMF (5 ml) and THF (2.5 ml) and cooled in an ice/water bath. After a few minutes DIPEA (0.25 ml, 1.42 mmol, 4 eq) and zinc triflate (6 eq) were added with magnetic stirring, then NCA (183 mg, 1.42 mmol, 4 eq) was added and the reaction was stirred under argon at room temperature for 17 hours. The reaction was diluted with EtOAC (20 mL) and treated with a solution of brine: saturated sodium bicarbonate (1:1) (4.4 mL), the resulting solution was stirred for 10 minutes. The white precipitate was filtered off, and the resulting aqueous solution was re-extracted with EtOAC (20 mL×2), and then organic layer was washed with brine. The resulting organic layer was concentrated to afford crude product for the next step without further purification (210 mg, ˜91% yield, 87% pure by HPLC). ESI m/z calcd. for C₃₂H₄₅ClN₃O₉ [M+H]⁺: 650.28, found: 650.29.

### Example 49. Synthesis of (S)-tert-butyl 34-(((benzyloxy)carbonyl)amino)-28,35-dioxo-2,5,8,11,14,17,20,23,26-nonaoxa-29,36-diazatetracontan-40-oate (210)

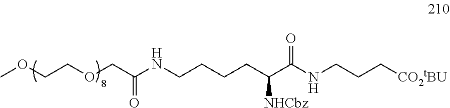

A mixture of tert-butyl 4-aminobutanoate (1.03 g, 6.12 mmol) and compound 4 (3.91 g, 5.56 mmol) in DMF (18 mL) at 0° C., HATU (2.32 g, 6.12 mmol) and TEA (1.2 mL, 8.34 mmol) were added in sequence. The reaction was stirred for 1 h, then diluted with water (300 mL), and extracted with ethyl acetate (3×250 mL). The organic solution was washed with brine, dried over anhydrous sodium sulfate, filtered, concentrated and purified by silica gel column chromatography (32:1 dichloromethane/methanol) to give the title compound (210) (5.10 g, 99% yield). ESI MS m/z 846.50 ([M+H]⁺).

### Example 50. Synthesis of (S)-tert-butyl 34-amino-28,35-dioxo-2,5,8,11,14,17,20,23,26-nonaoxa-29,36-diazatetracontan-40-oate (211)

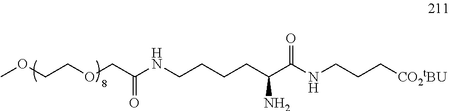

Compound 210 (1.0 g, 1.18 mmol) and Pd/C (10 wt %, 0.10 g) were added in a hydrogenation bottle having methanol (50 mL). The mixture was shaken for 2 h, filtered through Celite (filter aid), and the filtrate was concentrated to afford compound 211 (0.93 g, yield>100%). ESI MS m/z 712.50 ([M+H]⁺).

### Example 51. Synthesis of (S)-tert-butyl 34-(4-(2,5-dioxo-2,5-dihydro-1H-pyrrol-1-yl)butanamido)-28,35-dioxo-2,5,8,11,14,17,20,23,26-nonaoxa-29,36-diazatetracontan-40-oate (212)

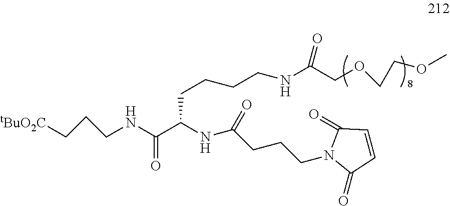

To a solution of compound 211 (0.93 g, 1.18 mmol) in 95% EtOH (50 mL) and NaH₂PO₄ solution (0.1 M, pH 5.0, 10 mL), N-succinimidyl 4-maleimido-butyrate (0.50 g, 1.77 mmol, 1.5 eq) was added. The mixture was stirred overnight, then concentrated and diluted with water (50 mL) and extracted with dichloromethane (80 mL×3), dried over anhydrous sodium sulfate, filtered, concentrated and purified by silica gel column chromatography (25:1 dichloromethane/methanol) to give the title compound as a light yellow oil (0.82 g, 80%). ESI MS m/z 877.52 ([M+H]⁺).

### Example 52. Synthesis of (S)-34-(4-(2,5-dioxo-2,5-dihydro-1H-pyrrol-1-yl)butanamido)-28,35-dioxo-2,5,8,11,14,17,20,23,26-nonaoxa-29,36-diazatetracontan-40-oic acid (213)

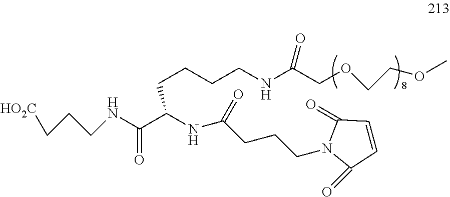

Compound 212 (0.82 g, 0.94 mmol) was dissolved in HCOOH (50 mL) and stirred at room temperature for 1 hour. The reaction mixture was concentrated and co-evaporated with toluene twice, and the residue was placed on a vacuum pump to give compound 213 (0.80 g, crude product). ESI MS m/z 820.45 ([M+H]⁺).

### Example 53. Synthesis of (S)-2,5-dioxopyrrolidin-1-yl 34-(4-(2,5-dioxo-2,5-dihydro-1H-pyrrol-1-yl)butanamido)-28,35-dioxo-2,5,8,11,14,17,20,23,26-nonaoxa-29,36-diazatetracontan-40-oate (214)

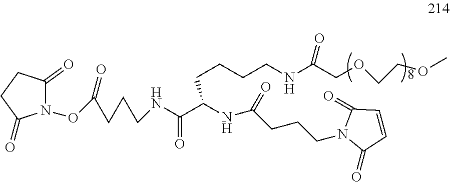

To a solution of compound 213 (0.80 g, crude, 0.94 mmol) in DMA (5.0 mL), NHS (0.12 g, 1.03 mmol) and EDC⋅HCl (0.27 g, 1.41 mmol) were added, and the reaction was stirred at r.t. for 2 h, then diluted with water (15 mL) and extracted with ethyl acetate (3×10 mL). The combined organic phase was washed with brine (10 mL), dried over anhydrous sodium sulfate, filtered and concentrated. The residue was purified by silica gel column (10-50% ethyl acetate/petroleum ether) to give a colorless oil compound (0.67 g, 78% yield). ESI MS m/z 918.55 ([M+H]⁺).

### Example 54. Synthesis of tert-butyl (2-(2,5-dioxo-2,5-dihydro-1H-pyrrol-1-yl)ethyl)carbamate (215)

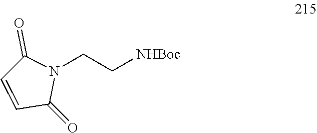 [SMILES: CC(C)(C)OC(=O)NCCN1C(=O)C=CC1=O]

A mixture of N-Boc-ethylenediamine (5.6 mL, 35.4 mmol, 1.1 eq.) and saturated NaHCO₃ (60 mL) was cooled to 0° C., to which N-methoxycarbonyl maleimide (5.00 g, 32.2 mmol, 1.0 eq.) was added in portions. After stirring at 0° C. for 30 min, the reaction was warmed to r.t. and stirred for 1 h. The precipitate was collected by filtration and washed with cold water, then dissolved in ethyl acetate and washed with brine, dried over anhydrous sodium sulfate and concentrated to give a white solid (6.69 g, 87% yield). ESI MS m/z 241.12 ([M+H]⁺).

### Example 55. Synthesis of tert-butyl (2-(1,3-dioxo-3a,4,7,7a-tetrahydro-1H-4,7-epoxyisoindol-2(3H)-yl)ethyl)carbamate (216)

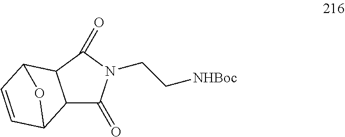 [SMILES: CC(C)(C)OC(=O)NCCN1C(=O)C2C3C=CC(O3)C2C1=O]

In a high pressure tube, a solution of compound 215 (6.00 g, 25.0 mmol), furan (18.0 mL) in toluene (120 mL) was heated to reflux and stirred for 16 h. The colorless solution turned yellow during reaction. The mixture was then cooled to r.t. and concentrated. The resulting white solid was triturated with ethyl ether to give compound 216 (6.5 g, 84% yield). ESI MS m/z 309.13 ([M+H]⁺).

### Example 57. Synthesis of 2-(2-aminoethyl)-3a,4,7,7a-tetrahydro-1H-4,7-epoxyisoindole-1,3(2H)-dione hydrochloride (217)

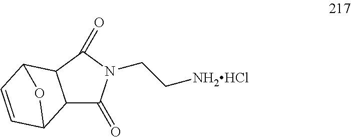

A solution of compound 216 (9.93 g, 32.2 mmol) in dioxane (15 mL) was treated with concentrated HCl (15 mL) at r.t. for 3 h. The reaction was concentrated and the resulting solid was collected by filtration, with washing of the filter cake with ethyl acetate. The solid was dried in an oven (50° C.) overnight to give compound 217 (6.94 g, 88% yield). ESI MS m/z 206.05 ([M+H]⁺).

### Example 58. Synthesis of Compound 218

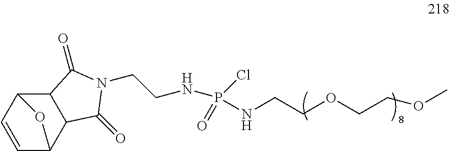

To a solution of compound 217 (1.22 g, 5 mmol) in THF (10 mL) at −10° C., POCl₃ (0.47 mL, 5 mmol) was added. After stirring for 10 min., 2,5,8,11,14,17,20,23,26-nonaoxaoctacosan-28-amine (2.14 g, 5 mmol) was added, followed by DIPEA (0.87 mL, 5 mmol). The reaction was warmed to 0° C. and stirred for 3 h, and then concentrated. The residue was diluted with dichloromethane (10 mL) and filtered over Celite, the filtrate was used in the next step directly. ESI MS m/z 716.29 ([M+H]⁺).

### Example 59. Synthesis of methyl 4-(bis(2-hydroxyethyl)amino)-4-oxobutanoate (219)

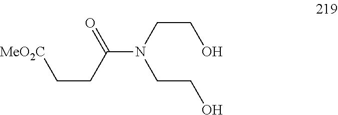 [SMILES: COC(=O)CCC(=O)N(CCO)CCO]

Dimethyl succinate (20.0 g, 136.9 mmol) and dihydroxyethylamine (7.20 g, 68.7 mmol) in a mixture of anhydrous toluene (500 ml) and pyridine (50 ml) were heated at 150° C. for 28 h. The mixture was concentrated and purified on silica gel column eluted with 5-25% ethyl acetate/dichloromethane to afford the title compound (12.5 g, 83% yield). ESI MS m/z 242.42 [M+Na]⁺.

### Example 60. Synthesis of methyl 4-(bis(2-((methylsulfonyl)oxy)ethyl) amino)-4-oxobutanoate (220)

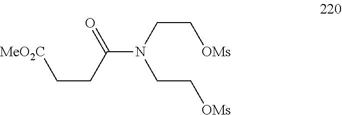 [SMILES: COC(=O)CCC(=O)N(CCOS(C)(=O)=O)CCOS(C)(=O)=O]

To a solution of methyl 4-(bis(2-hydroxyethyl)amino)-4-oxobutanoate (12.0 g, 49.56 mmol) in anhydrous pyridine (350 ml), methanesulfonyl chloride (20.0 g, 175.4 mmol) was added. After stirring overnight, the mixture was concentrated, diluted with ethyl acetate (350 ml), washed with cold 1 M NaH₂PO₄ (2×300 mL), dried over MgSO₄, filtered and evaporated to afford crude product (˜18.8 g, >100% yield). The crude product was used in the next step without further purification. ESI MS m/z 376.06 ([M+H]⁺).

### Example 61. Synthesis of 3,6-endoxo-Δ-tetrahydrophthalimide (221)

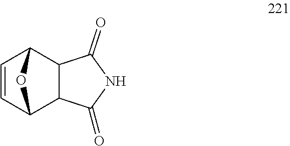 [SMILES: O=C1NC(=O)C2C1[C@@H]1C=C[C@H]2O1]

To a solution of maleimide (10.0 g, 103.0 mmol) in toluene (200 ml) was added furan (10.0 ml, 137.4 mmol). The mixture was heated in a 1 L auto Clave bomb at 100° C. for 8 h. The bomb was cooled to room temperature, and the solid was rinsed out with methanol, concentrated and crystallized in ethyl acetate/hexane to afford 16.7 g (99%) of the title compound. 1H NMR (CDCl₃): 11.12 (s, 1H), 6.68˜6.64 (m, 2H), 5.18˜5.13 (m, 2H), 2.97˜2.92 (m, 2H). ESI MS m/z [M+Na]⁺ 188.04.

### Example 62. Synthesis of Methyl 4-((2-((3aR,4R,7S,7aS)-1,3-dioxo-3a,4,7,7a-tetrahydro-1H-4,7-epoxyisoindol-2(3H)-yl)ethyl)(2-((4R,7S,7aS)-1,3-dioxo-3a,4,7,7a-tetrahydro-1H-4,7-epoxyisoindol-2(3H)-yl)ethyl)amino)-4-oxobutanoate (222)

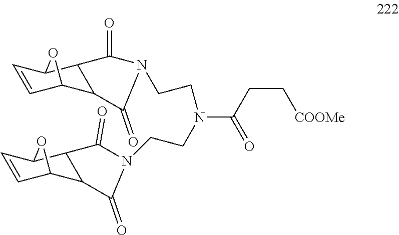 [SMILES: COC(=O)CCC(=O)N(CCN1C(=O)C2C3C=CC(O3)C2C1=O)CCN1C(=O)C2C3C=CC(O3)C2C1=O]

To a solution of methyl 4-(bis(2-((methylsulfonyl)oxy)ethyl)amino)-4-oxobutanoate (220, fresh made, 90% pure, 8.5 g, ˜20 mmol) in DMA (350 ml), 3,6-endoxo-A-tetrahydrophthalimide (10.2 g, 61.8 mmol), sodium carbonate (8.0 g, 75.5 mmol) and sodium iodide (0.3 g, 2.0 mmol) were added. The mixture was stirred at room temperature overnight, concentrated, diluted with ethyl acetate (350 ml), washed with sat'ed NaHCO₃ solution (300 ml), sat'ed NaCl solution (300 ml) and 1 M NaH₂PO₄ (300 ml). The organic layer was dried over sodium sulfate, filtered, evaporated, loaded on silica gel column and eluted with 10-30% ethyl acetate/hexane to afford the title compound (7.9 g, 77% yield). ESI MS m/z [M+Na]⁺ 536.4.

### Example 63. Synthesis of 4-(bis(2-(2,5-dioxo-2,5-dihydro-1H-pyrrol-1-yl)ethyl) amino)-4-oxobutanoic acid (223)

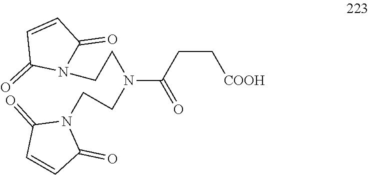 [SMILES: O=C(O)CCC(=O)N(CCN1C(=O)C=CC1=O)CCN1C(=O)C=CC1=O]

Compound 222 (3.0 g, 5.8 mmol) and trimethylstannanol (4.8 g, 26.4 mmol) in 1,2-dichloroethane (150 ml) were refluxed at 80° C. for 8 h, then cooled to room temperature and the residue was passed through a short silica gel column and eluted with dichloromethane/methanol to remove excess trimethyltin hydroxide. Then the pooled fractions were combined, concentrated and diluted with DMA and toluene, heated to 120° C. and stirred overnight. The reaction mixture was loaded on silica gel column and eluted with 5-10% methanol/dichloromethane to afford the title compound (1.62 g, 76% yield). ESI MS m/z [M+Na]⁺ 386.2.

### Example 64. Synthesis of (S)-tert-butyl 34-(4-(bis(2-(2,5-dioxo-2,5-dihydro-1H-pyrrol-1-yl)ethyl)amino)-4-oxobutanamido)-28,35-dioxo-2,5,8,11,14,17,20,23,26-nonaoxa-29,36-diazatetracontan-40-oate (224)

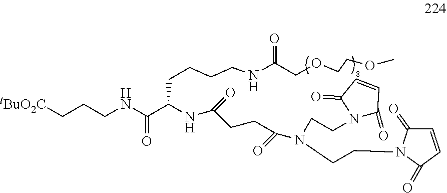

To a solution of compound 223 (1.62 g, 4.20 mmol) and compound 211 (2.71 g, 3.82 mmol) in DMA (20 mL), EDC⋅HCl (0.81 g, 4.20 mmol) was added. The reaction was stirred at r.t. overnight, then poured onto water (50 mL) and extracted with ethyl acetate (3×40 mL). The combined organic phase was washed with brine (40 mL), dried over anhydrous sodium sulfate, filtered and concentrated. The residue was purified by column chromatography (10-50% ethyl acetate/petroleum ether) to give a colorless oil (3.20 g, 80% yield). ESI MS m/z 1057.85 ([M+H]⁺).

### Example 65. Synthesis of (S)-34-(4-(bis(2-(2,5-dioxo-2,5-dihydro-1H-pyrrol-1-yl)ethyl)amino)-4-oxobutanamido)-28,35-dioxo-2,5,8,11,14,17,20,23,26-nonaoxa-29,36-diazatetracontan-40-oic acid (225)

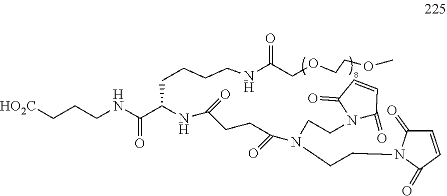

A solution of compound 224 (3.20 g, 3.03 mmol) in formic acid (10 mL) was stirred at r.t. overnight. The solution was then concentrated and co-evaporated with toluene three times to give a colorless oil (3.00 g, crude), which was used without further purification. ESI MS m/z 1001.50 ([M+H]⁺).

### Example 66. Synthesis of (S)-2,5-dioxopyrrolidin-1-yl 34-(4-(bis(2-(2,5-dioxo-2,5-dihydro-1H-pyrrol-1-yl)ethyl)amino)-4-oxobutanamido)-28,35-dioxo-2,5,8,11,14,17,20,23,26-nonaoxa-29,36-diazatetracontan-40-oate (226)

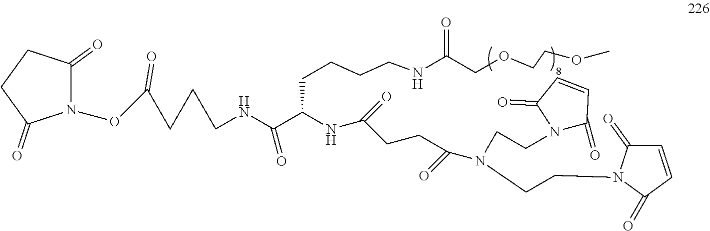

To a solution of compound 225 (3.00 g, crude, 3.03 mmol) in DMA (15.0 mL), NHS (0.38 g, 3.33 mmol) and EDC⋅HCl (0.87 g, 4.55 mmol) were added, and the reaction was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-50% ethyl acetate/petroleum ether) to give a colorless oil (2.90 g, 90% yield). ESI MS m/z 1098.50 ([M+H]⁺).

### Example 67. Synthesis of 14-(benzyloxy)-14-oxotetradecanoic acid (227)

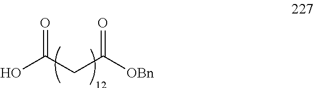

To a solution of tetradecanedioic acid (2.06 g, 8 mmol) in DMF (30 mL), K₂CO₃ (1.1 g, 8 mmol) and BnBr (1.36 g, 8 mmol) were added. The mixture was stirred at r.t. overnight, then concentrated and purified by column chromatography (ethyl acetate/petroleum ether) to afford the title compound 227 (1.2 g, 45% yield). ESI MS m/z 349.23 ([M+H]⁺).

### Example 68. Synthesis of tert-butyl 3-(2-(2-(2-hydroxyethoxy)ethoxy)ethoxy) propanoate (228)

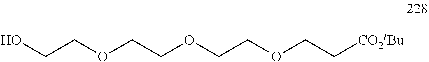 [SMILES: CC(C)(C)OC(=O)CCOCCOCCOCCO]

To a solution of 2,2′-(ethane-1,2-diylbis(oxy))diethanol (55.0 mL, 410.75 mmol, 3.0 eq.) in anhydrous THF (200 mL), sodium (0.1 g) was added. The mixture was stirred until Na disappeared and then tert-butyl acrylate (20.0 mL, 137.79 mmol, 1.0 eq.) was added dropwise. The mixture was stirred overnight and then quenched by HCl solution (20.0 mL, 1N) at 0° C. THF was removed by rotary evaporation, brine (300 mL) was added and the resulting mixture was extracted with ethyl acetate (3×100 mL). The organic layers were washed with brine (3×300 mL), dried over anhydrous sodium sulfate, filtered and concentrated to afford a colorless oil (30.20 g, 79.0% yield), which was used without further purification. MS ESI m/z 278.17 ([M+H]⁺).

### Example 69. Synthesis of tert-butyl 3-(2-(2-(2-(tosyloxy)ethoxy)ethoxy)ethoxy) propanoate (229)

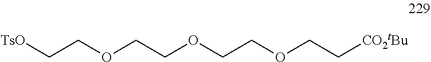 [SMILES: Cc1ccc(S(=O)(=O)OCCOCCOCCOCCC(=O)OC(C)(C)C)cc1]

To a solution of tert-butyl 3-(2-(2-(2-hydroxyethoxy)ethoxy)ethoxy) propanoate (30.20 g, 108.5 mmol, 1.0 eq.) and TsCl (41.37 g, 217.0 mmol, 2.0 eq.) in anhydrous DCM (220 mL) at 0° C., TEA (30.0 mL, 217.0 mmol, 2.0 eq.) was added. The mixture was stirred at room temperature overnight, and then washed with water (3×300 mL) and brine (300 mL), dried over anhydrous sodium sulfate, filtered, concentrated and purified by silica gel column chromatography (3:1 hexanes/ethyl acetate) to give a colorless oil (39.4 g, 84.0% yield). MS ESI m/z 433.28 ([M+H]⁺).

### Example 70. Synthesis of tert-butyl 3-(2-(2-(2-azidoethoxy)ethoxy)ethoxy) propanoate (230)

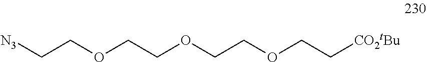 [SMILES: CC(C)(C)OC(=O)CCOCCOCCOCCN=[N+]=[N-]]

To a solution of tert-butyl 3-(2-(2-(2-(tosyloxy)ethoxy)ethoxy)ethoxy) propanoate (39.4 g, 91.1 mmol, 1.0 eq.) in anhydrous DMF (100 mL), NaN₃ (20.67 g, 316.6 mmol, 3.5 eq.) was added. The mixture was stirred at room temperature overnight. Water (500 mL) was added and extracted with ethyl acetate (3×300 mL). The combined organic layers were washed with water (3×900 mL) and brine (900 mL), dried over anhydrous sodium sulfate, filtered, concentrated and purified by silica gel column chromatography (5:1 hexanes/ethyl acetate) to give a light yellow oil (23.8 g, 85.53% yield). MS ESI m/z 326.2 ([M+Na]+).

### Example 71. Synthesis of tert-butyl 3-(2-(2-(2-aminoethoxy)ethoxy)ethoxy) propanoate (231)

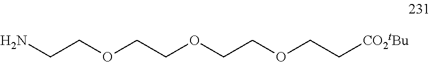 [SMILES: CC(C)(C)OC(=O)CCOCCOCCOCCN]

Raney-Ni (7.5 g, suspended in water) was washed with water (three times) and isopropyl alcohol (three times) and mixed with compound 230 (5.0 g, 16.5 mmol) in isopropyl alcohol. The mixture was stirred under a H₂ balloon at r.t. for 16 h and then filtered over a Celite pad, with washing of the pad with isopropyl alcohol. The filtrate was concentrated and purified by column chromatography (5-25% methanol/dichloromethane) to give a light yellow oil (2.60 g, 57% yield). MS ESI m/z 279.19 ([M+H]⁺).

### Example 72. Synthesis of 27-benzyl 1-tert-butyl 14-oxo-4,7,10-trioxa-13-azaheptacosane-1,27-dioate (232)

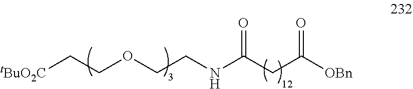

To a solution of compound 231 (2.60 g, 9.35 mmol) and compound 227 (3.91 g, 11.2 mmol) in dichloromethane (50 mL), EDC⋅HCl (2.15 g, 11.2 mmol) and DIPEA (3.6 mL, 20.6 mmol) were added. The reaction mixture was stirred at r.t. for 1 h, then diluted with 50 mL dichloromethane and poured into a separatory funnel containing 50 mL of water. The organic phase was separated, washed with brine (50 mL), dried over anhydrous sodium sulfate, filtered and concentrated. The residue was purified by column chromatography (0-10% methanol/dichloromethane) to afford the title compound 232 (4.94 g, 87% yield). ESI m/z 608.40 ([M+H]⁺).

### Example 73. Synthesis of 3,16-dioxo-1-phenyl-2,20,23,26-tetraoxa-17-azanonacosan-29-oic acid (233)

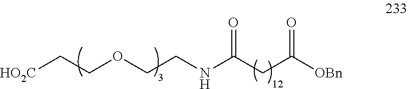

To a solution of compound 232 (4.94 g, 8.14 mmol) in dichloromethane (20 mL), TFA (20 mL) was added. The reaction was stirred at room temperature for 1 h, then concentrated to dryness and co-evaporated twice with dichloromethane, and the residue was placed on a pump to give compound 233 (4.50 g, crude product). ESI MS m/z 552.35 ([M+H]⁺).

### Example 74. Synthesis of 40-benzyl 1-tert-butyl 14,27-dioxo-4,7,10,17,20,23-hexaoxa-13,26-diazatetracontane-1,40-dioate (234)

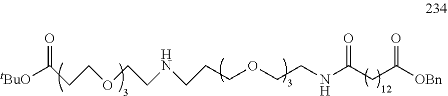

To a solution of compound 233 (4.50 g, crude, 8.14 mmol) and compound 231 (1.95 g, 7.00 mmol) in dichloromethane (50 mL), EDC⋅HCl (1.56 g, 8.14 mmol) and DIPEA (2.7 mL, 15.4 mmol) were added. The reaction mixture was stirred at r.t. for 1 h, then diluted with 50 mL dichloromethane and poured into a separatory funnel containing 50 mL of water. The organic phase was separated, washed with brine (50 mL), dried over anhydrous sodium sulfate, filtered and concentrated. The residue was purified by column chromatography (0-10% methanol/dichloromethane) to afford the title compound 234 (5.22 g, 92% yield). ESI m/z 811.52 ([M+H]⁺).

### Example 75. Synthesis of 3,16,29-trioxo-1-phenyl-2,20,23,26,33,36,39-heptaoxa-17,30-diazadotetracontan-42-oic acid (235)

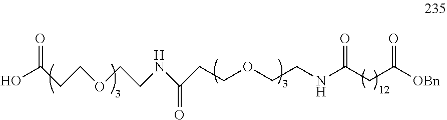

To a solution of compound 234 (5.22 g, 6.44 mmol) in dichloromethane (20 mL), TFA (5 mL) was added. The reaction was stirred at room temperature for 1 h, then concentrated to dryness and co-evaporated twice with dichloromethane, and the residue was placed on a pump to give compound 235 (4.90 g, crude product). ESI MS m/z 755.46 ([M+H]⁺).

### Example 76. Synthesis of 40-benzyl 1-(2,5-dioxopyrrolidin-1-yl) 14,27-dioxo-4,7,10,17,20,23-hexaoxa-13,26-diazatetracontane-1,40-dioate (236)

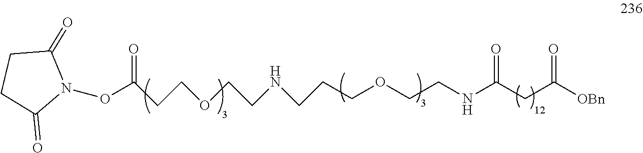

To a solution of compound 235 (4.90 g, crude, 6.44 mmol) in dichloromethane (30 mL), NHS (0.81 g, 7.08 mmol), EDC⋅HCl (1.85 g, 9.66 mmol), and DIPEA (2.8 mL, 16.1 mmol) were added. The reaction mixture was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified by silica gel column (10-50% ethyl acetate/petroleum ether) to give a colorless oil 236 (4.90 g, 90% yield). ESI MS m/z 852.48 ([M+H]⁺).

### Example 77. Synthesis of 1-((2,5-dioxopyrrolidin-1-yl)oxy)-1,14,27-trioxo-4,7,10,17,20,23-hexaoxa-13,26-diazatetracontan-40-oic acid (237)

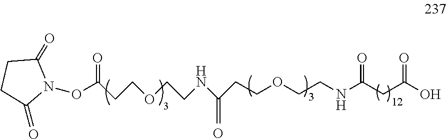

To a solution of compound 193 (4.90 g, 5.75 mmol) in THF (20 mL) in a hydrogenation bottle, Pd/C (10 wt %, 0.20 g) was added. The mixture was stirred under 1 atm H₂ overnight, filtered through Celite (filter aid), and the filtrated solution was concentrated to afford compound 237 (4.50 g, >100% yield). ESI MS m/z 762.44 ([M+H]⁺).

### Example 78. Synthesis of (6S,13S)-di-tert-butyl 9,10-bis(((benzyloxy)carbonyl)amino)-5,8,11,14-tetraoxo-6,13-bis(4-(((2-(trimethylsilyl)ethoxy)carbonyl)amino)butyl)-4,7,12,15-tetraazaoctadecane-1,18-dioate (152)

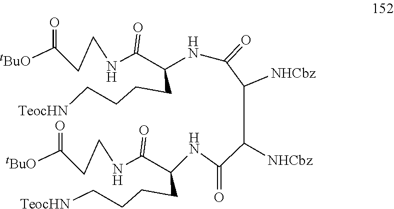

To a solution of (S)-tert-butyl 12-amino-2,2-dimethyl-6,13-dioxo-5-oxa-7,14-diaza-2-silaheptadecan-17-oate (6.02 g, 14.4 mmol) and 2,3-bis(((benzyloxy)carbonyl)amino)succinic acid (5.00 g, 12.0 mmol) in DMA (60 mL), EDC⋅HCl (2.76 g, 14.4 mmol) and DIPEA (4.7 mL, 26.4 mmol) were added. The reaction mixture was stirred at r.t. overnight, then diluted with 150 mL of dichloromethane and poured into a separatory funnel containing 100 mL of water. The organic phase was separated, washed with brine (2×50 mL), dried over anhydrous sodium sulfate, filtered and concentrated. The residue was purified by column chromatography (10-80% ethyl acetate/petroleum ether) to afford the title compound 152 (12.4 g, 85% yield). ESI MS m/z 1215.63 ([M+H]⁺).

### Example 79. Synthesis of (65,13 S)-di-tert-butyl 9,10-diamino-5,8,11,14-tetraoxo-6,13-bis(4-(((2-(trimethylsilyl)ethoxy)carbonyl)amino)butyl)-4,7,12,15-tetraazaoctadecane-1,18-dioate (153)

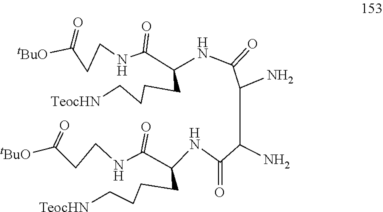

To a solution of compound 152 (12.4 g, 10.2 mmol) and Pd/C (10 wt %, 0.10 g) in methanol (50 mL) in a hydrogenation bottle, hydrogen (5 psi) was conducted in. The mixture was shaken for 2 h, filtered through Celite (filter aid), and the filtrate was concentrated to afford compound 153 (9.47 g, 98% yield) as a colorless oil. ESI MS m/z 947.56 ([M+H]⁺).

### Example 80. Synthesis of (6S,13S)-di-tert-butyl 9,10-bis(3-(2,5-dioxo-2,5-dihydro-1H-pyrrol-1-yl)propanamido)-5,8,11,14-tetraoxo-6,13-bis(4-(((2-(trimethylsilyl)ethoxy)carbonyl)-amino)butyl)-4,7,12,15-tetraazaoctadecane-1,18-dioate (154)

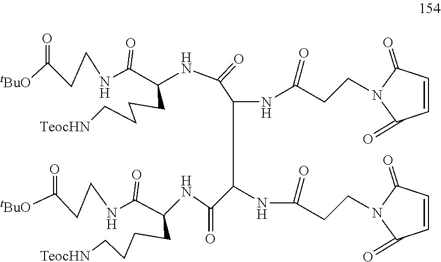

To a solution of compound 153 (9.47 g, 10.0 mmol) in dichloromethane (50 mL), NHS (1.39 g, 12.0 mmol), EDC⋅HCl (2.30 g, 12.0 mmol) and DIPEA (3.8 mL, 22.0 mmol) were added. The reaction mixture was stirred at r.t. for 2 h, then diluted with water (50 mL) and extracted with ethyl acetate (3×30 mL). The combined organic phase was washed with brine (30 mL), dried over anhydrous sodium sulfate, filtered and concentrated. The residue was purified on silica gel column (10-80% ethyl acetate/petroleum ether) to give a colorless oil (9.49 g, 76% yield). ESI MS m/z 1249.72 ([M+H]⁺).

### Example 81. Synthesis of (6S,13S)-di-tert-butyl 6,13-bis(4-aminobutyl)-9,10-bis(3-(2,5-dioxo-2,5-dihydro-1H-pyrrol-1-yl)propanamido)-5,8,11,14-tetraoxo-4,7,12,15-tetraazaoctadecane-1,18-dioate (155)

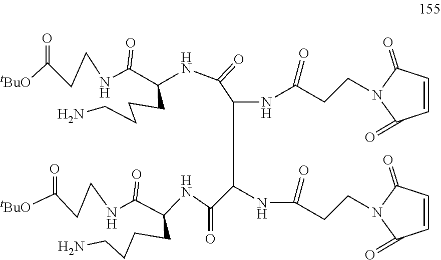 [SMILES: CC(C)(C)OC(=O)CCNC(=O)[C@H](CCCCN)NC(=O)C(NC(=O)CCN1C(=O)C=CC1=O)C(NC(=O)CCN1C(=O)C=CC1=O)C(=O)N[C@@H](CCCCN)C(=O)NCCC(=O)OC(C)(C)C]

To a solution of compound 154 (8.50 g, 6.80 mmol) in methanol (100 mL), NH₄F (0.80 g, 21.62 mmol) and a drop of 1.0 M HCl (˜0.010 ml) were added. The reaction was kept for stirring at r.t for 2 h, following by 50° C. for 2 h. The mixture was then diluted with DMF (30 ml), evaporated in vacuo and dried with oil vacuum pump to give the crude product (8.19 g, >100% yield) for next step without further purification. ESI MS m/z 961.53 ([M+H]⁺).

### Example 82. Synthesis of (6S,13S)-di-tert-butyl 9,10-bis(3-(2,5-dioxo-2,5-dihydro-1H-pyrrol-1-yl)propanamido)-5,8,11,14-tetraoxo-6,13-bis(29-oxo-2,5,8,11,14,17,20,23,26-nonaoxa-30-azatetratriacontan-34-yl)-4,7,12,15-tetraazaoctadecane-1,18-dioate (157)

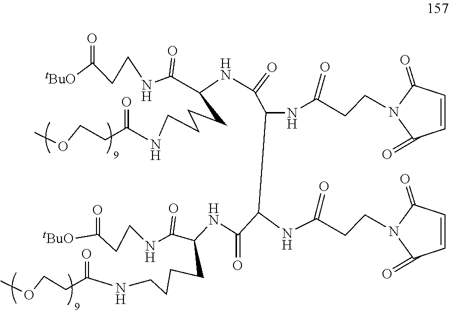

To a solution of the crud compound 155 (8.19 g, ˜ 6.80 mmol) in DMA (100 mL), 2,5,8,11,14,17,20,23,26-nonaoxanonacosan-29-oic acid (6.92 g, 15.17 mmol) and EDC⋅HCl (6.30 g, 33.15 mmol) were added. The reaction mixture was stirred at r.t. for 8 h, then concentrated, diluted with water (50 mL) and extracted with ethyl acetate (3×80 mL). The combined organic phase was dried over anhydrous sodium sulfate, filtered, concentrated and purified by silica gel column (10%-30% MeOH/DCM) to give a colorless oil (6.51 g, 52% yield in two steps). ESI MS m/z 1839.09 ([M+H]⁺).

### Example 83. Synthesis of (6S,13S)-9,10-bis(3-(2,5-dioxo-2,5-dihydro-1H-pyrrol-1-yl)propanamido)-5,8,11,14-tetraoxo-6,13-bis(29-oxo-2,5,8,11,14,17,20,23,26-nonaoxa-30-azatetratriacontan-34-yl)-4,7,12,15-tetraazaoctadecane-1,18-dioic acid (158)

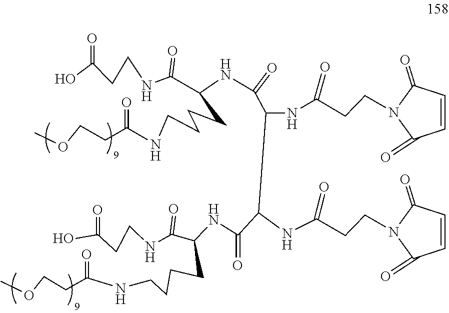

A solution of compound 157 (6.49 g, 3.53 mmol) in dioxane (30 mL) was treated with concentrated HCl (10 mL) at 0° C. for 30 min, then diluted with toluene (50 ml), concentrated and purified on a short silica gel column with elution of 10-25% methanol/dichloromethane to give the colorless oil product (5.47 g, 90% yield). ESI MS m/z 1725.88 ([M+H]⁺).

### Example 84. Synthesis of (18S,25S)-di-tert-butyl 21,22-bis(3-(2,5-dioxo-2,5-dihydro-1H-pyrrol-1-yl)propanamido)-4,7,10,13,17,20,23,26,30,33,36,39-dodecaoxo-18,25-bis(29-oxo-2,5,8,11,14,17,20,23,26-nonaoxa-30-azatetratriacontan-34-yl)-3,6,9,12,16,19,24,27,31,34,37,40-dodecaazadotetracontane-1,42-dioate (160)

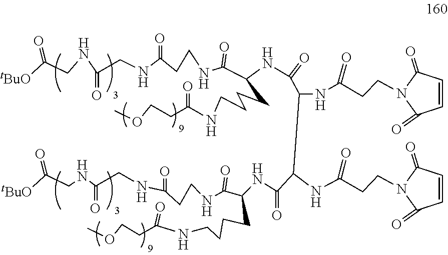

To a solution of the compound 158 (5.40 g, 3.13 mmol) in DMA (100 mL), tert-butyl 2-(2-(2-(2-aminoacetamido)acetamido)acetamido)acetate (gly-gly-gly-gly-O^{t}Bu) (2.50 g, 8.27 mmol) and EDC⋅HCl (5.50 g, 28.94 mmol) were added. The reaction mixture was stirred at r.t. for 8 h, then concentrated, diluted with water (50 mL) and extracted with ethyl acetate (3×80 mL). The combined organic phase was dried over anhydrous sodium sulfate, filtered, concentrated and purified by silica gel column (5%-20% MeOH/DCM) to give a colorless oil (5.95 g, 83% yield). ESI MS m/z 2294.52 ([M+H]⁺).

### Example 85. Synthesis of (18S,25S)-21,22-bis(3-(2,5-dioxo-2,5-dihydro-1H-pyrrol-1-yl)propanamido)-4,7,10,13,17,20,23,26,30,33,36,39-dodecaoxo-18,25-bis(29-oxo-2,5,8,11,14,17,20,23,26-nonaoxa-30-azatetratriacontan-34-yl)-3,6,9,12,16,19,24,27,31,34,37,40-dodecaazadotetracontane-1,42-dioic acid (161)

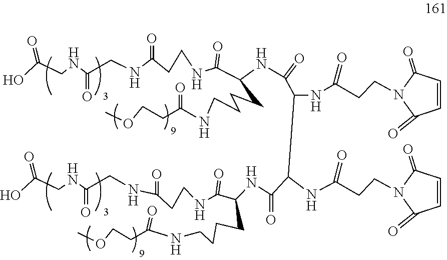

A solution of compound 160 (5.90 g, 2.57 mmol) in dioxane (30 mL) was treated with concentrated HCl (10 mL) at 0° C. for 30 min, then diluted with toluene (50 ml), concentrated and loaded on a short silica gel column and eluted with 10-30% methanol/dichloromethane to give a colorless oil (4.60 g, 82% yield). ESI MS m/z 2182.33 ([M+H]⁺).

### Example 86. Synthesis of bis((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) ((35S,35'S)-35,35′-((2,3-bis(3-(2,5-dioxo-2,5-dihydro-1H-pyrrol-1-yl)propanamido)succinyl)bis(azanediyl))-bis(29,36,40,43,46,49,52-heptaoxo-2,5,8,11,14,17,20,23,26-nonaoxa-30,37,41,44,47,50,53-heptaazapentapentacontane-55,35-diyl))dicarbamate (173)

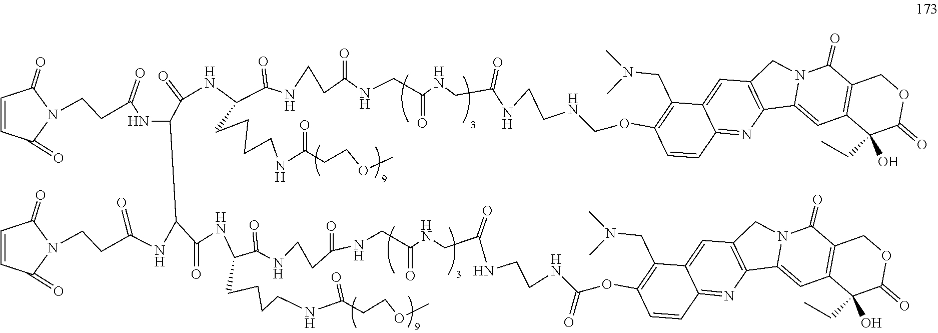

To a solution of the compound 161 (180.5 mg, 0.0825 mmol) in DMA (6 mL), (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2-aminoethyl)carbamate HCl salt (172) (145.0 mg, 0.267 mmol), EDC⋅HCl (120.2 mg, 0.632 mmol) and DIPEA (0.10 ml, 0.57 mmol) were added. The reaction mixture was stirred at r.t. for 8 h, then concentrated, diluted with water (50 mL) and extracted with ethyl acetate (3×30 mL). The combined organic phase was dried over anhydrous sodium sulfate, filtered, concentrated and purified by silica gel column (5%-15% MeOH/DCM) to give a colorless oil (212.3 mg, 82% yield). ESI MS m/z 3160.89 ([M+H]⁺).

### Example 87. Synthesis of 2,3-bis(3-(2,5-dioxo-2,5-dihydro-1H-pyrrol-1-yl)propanamido)-N1,N4-bis((35S)-52-(((9R)-9-ethyl-5-fluoro-9-hydroxy-4-methyl-10,13-dioxo-1,2,3,9,10,12,13,15-octahydrobenzo[de]pyrano[3′,4′:6,7]indolizino[1,2-b]quinolin-1-yl)amino)-29,36,40,43,46,49,52-heptaoxo-2,5,8,11,14,17,20,23,26-nonaoxa-30,37,41,44,47,50-hexaazadopentacontan-35-yl)succinamide (238)

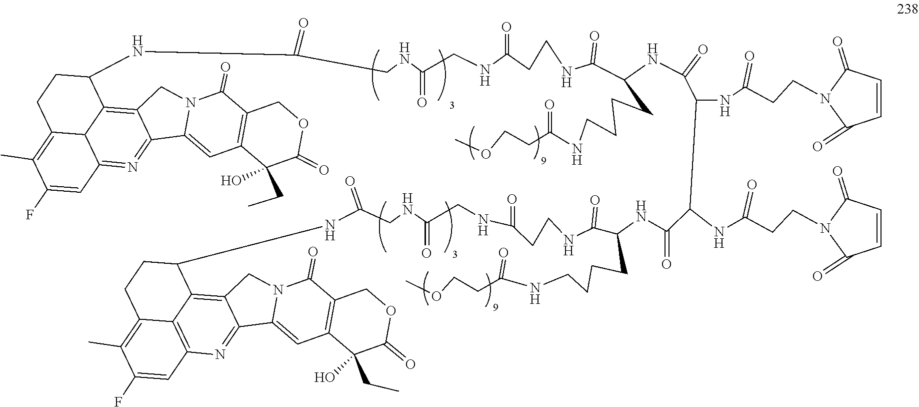

To a solution of the compound 161 (195.1 mg, 0.0894 mmol) in DMA (6 mL), (9R)-1-amino-9-ethyl-5-fluoro-9-hydroxy-4-methyl-2,3,12,15-tetrahydrobenzo[de]pyrano-[3′,4′:6,7]indolizino[1,2-b]quinoline-10,13(1H,9H)-dione HCl salt (65) (128.0 mg, 0.271 mmol), EDC⋅HCl (120.0 mg, 0.632 mmol) and DIPEA (0.10 ml, 0.57 mmol) were added. The reaction mixture was stirred at r.t. for 8 h, then concentrated, diluted with water (50 mL) and extracted with ethyl acetate (3×30 mL). The combined organic phase was dried over anhydrous sodium sulfate, filtered, concentrated and purified by silica gel column (5%-15% MeOH/DCM) to give a colorless oil (215.5 mg, 80% yield). ESI MS m/z 3016.38 ([M+H]⁺).

### Example 88. Synthesis of tert-butyl (2-isocyanatoethyl)carbamate (239)

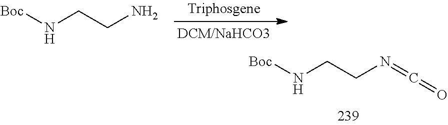 [SMILES: CC(C)(C)OC(=O)NCCN ; CC(C)(C)OC(=O)NCCN=C=O]

N-tert-butoxycarbonyl-1,2-ethanediamine (10.0 g, 0.062 mol) in a mixture of dichloromethane/saturated NaHCO₃ solution (100 mL/100 mL) at about 0° C., triphosgene (6.1 g, 0.02 mol) was added at one time. After the addition, the reaction was stirred at 0° C. for 1 hour. Two phases were separated and the dichloromethane phase was washed with water (30 mL), brine (30 mL), dried over sodium sulfate, filtered and concentrated to afford compound 239 (8.6 g, 74% yield).

### Example 89. Synthesis of tert-butyl 2-isocyanatoacetate (240)

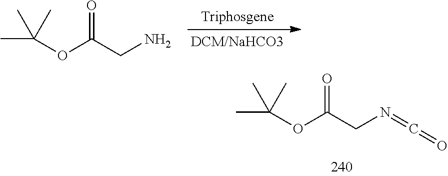

Glycine tert-butyl ester hydrochloride (10.0 g, 0.059 mol) in a mixture of dichloromethane/saturated NaHCO₃ solution (100 mL/100 mL) at about 0° C., triphosgene (5.9 g, 0.19 mol) was added at one time. After the addition, the reaction was stirred at 0° for 1 hour. Two phases were separated and the dichloromethane phase was washed with water (30 mL), brine (30 mL), dried over sodium sulfate, filtered and concentrated. The crude product was distilled (2 torr, 35° C.) to afford the colorless oil product (6.1 g, 65% yield).

### Example 90. Synthesis of (S)-tert-butyl (10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) ethane-1,2-diyldicarbamate (241)

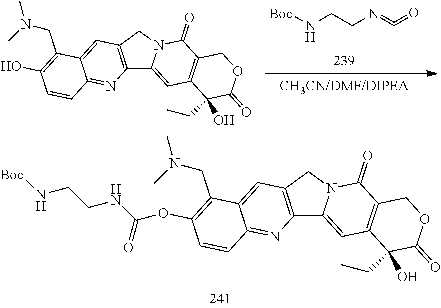

Topotecan (50 mg, 0.109 mmol) in a mixed solution of DMF/CH₃CN (1 mL/3 mL) at 0° C., DIPEA (34 mg, 0.27 mmol) and compound 239 (30 mg, 0.164 mmol) were added. The reaction mixture was stirred at 0° C. for 1 hour, then room temperature for another 1 hour, concentrated to dryness, triturated with 4 mL of ethyl acetate to give a yellow solid (241) (47 mg, 69% yield). MS-ESI m/z: [M+H]⁺ calcd for C₃₁H₃₇N₅O₈, 608.26; found, 608.26.

### Example 91. Synthesis of (S)-tert-butyl 2-((((10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)oxy)carbonyl)amino)acetate (242)

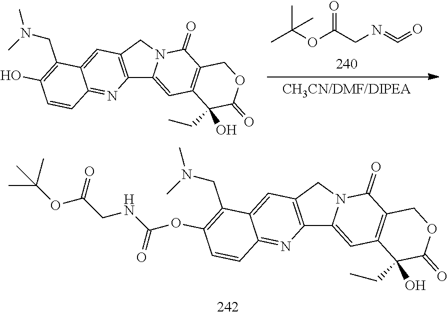

Topotecan (50 mg, 0.109 mmol) in a mixed solution of DMF/CH₃CN (1 mL/3 mL) at 0° C., DIPEA (34 mg, 0.27 mmol) and compound 240 (26 mg, 0.164 mmol) were added in sequence. The reaction mixture was stirred at 0° C. for 1 hour, then room temperature for another 1 hour, concentrated to dryness, triturated with 4 mL of ethyl acetate to give a yellow solid (242) (43 mg, 68% yield). MS-ESI m/z: [M+H]⁺ calcd for C₃₀H₃₄N₄O₈, 579.24; found, 579.24.

### Example 92. Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2-aminoethyl)carbamate (243)

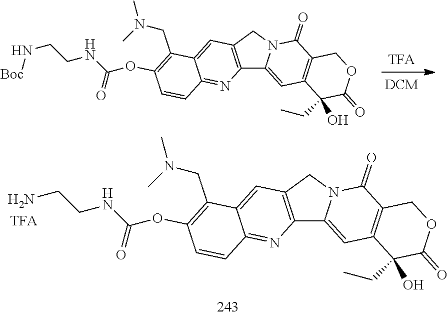 [SMILES: CC[C@@]1(O)C(=O)OCc2c1cc1n(c2=O)Cc2cc3c(CN(C)C)c(OC(=O)NCCNC(=O)OC(C)(C)C)ccc3nc2-1 ; CC[C@@]1(O)C(=O)OCc2c1cc1n(c2=O)Cc2cc3c(CN(C)C)c(OC(=O)NCCN)ccc3nc2-1]

Compound 241 (47 mg, 0.077 mmol) suspended in dichloromethane (3 mL) with stirring, TFA (1 mL) was added. The solution turned clear. After stirred for 0.5 hour, the mixture was diluted with toluene (5 mL) and evaporated to give the title compound 243 (47 mg, 100% yield). MS-ESI m/z: [M+H]⁺ calcd for C₂₆H₂₉N₅O₆, 508.21; found, 508.21.

### Example 93. Synthesis of (S)-2-((((10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl)oxy)carbonyl)amino)acetic acid (244)

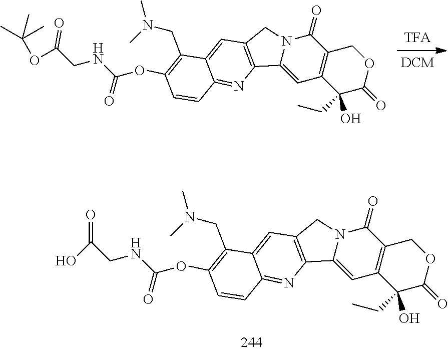 [SMILES: CC[C@@]1(O)C(=O)OCc2c1cc1n(c2=O)Cc2cc3c(CN(C)C)c(OC(=O)NCC(=O)OC(C)(C)C)ccc3nc2-1 ; CC[C@@]1(O)C(=O)OCc2c1cc1n(c2=O)Cc2cc3c(CN(C)C)c(OC(=O)NCC(=O)O)ccc3nc2-1]

Compound 242 (43 mg, 0.074 mmol) suspended in dichloromethane (3 mL) with stirring, TFA (1 mL) was added. After stirred for 0.5 hour, the mixture was diluted with toluene (5 mL) and evaporated to give the title compound 244 (39 mg, 100% yield). MS-ESI m/z: [M+H]⁺ calcd for C₂₆H₂₆N₄O₈ 523.18, found 523.18.

### Example 94. Synthesis of (S)-perfluorophenyl 30-(4-(2,5-dioxo-2,5-dihydro-1H-pyrrol-1-yl)butanamido)-27-oxo-2,5,8,11,14,17,20,23-octaoxa-26-azahentriacontan-31-oate

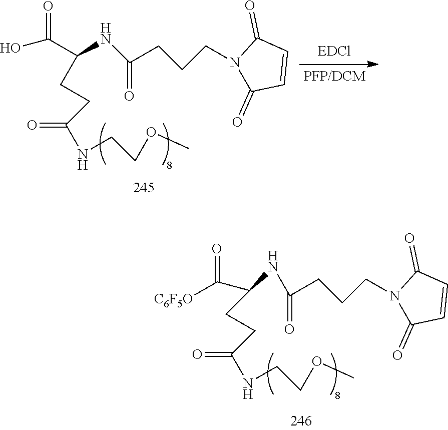

To (S)-30-(4-(2,5-dioxo-2,5-dihydro-1H-pyrrol-1-yl)butanamido)-27-oxo-2,5,8,11,14,17,20,23-octaoxa-26-azahentriacontan-31-oic acid (20 mg, 0.029 mmol) in dichloromethane (5 ml), EDC (11 mg, 0.059 mmol) and pentafluorophenol (10.8 mg, 0.059 mmol) were added. The reaction mixture was stirred at room temperature for 2 hours, concentrated and purified on SiO₂ column with elution of EtOAc/DCM (1:4) to give the title compound 246 (24 mg, 100% yield). MS-ESI m/z: [M+H]⁺ calcd for C₃₆H₅₀F₅N₃O₁₄, 844.32; found, 844.32.

### Example 95. Synthesis of(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl ((S)-30-(4-(2,5-dioxo-2,5-dihydro-1H-pyrrol-1-yl)butanamido)-27,31-dioxo-2,5,8,11,14,17,20,23-octaoxa-26,32-diazatetratriacontan-34-yl)carbamate (247)

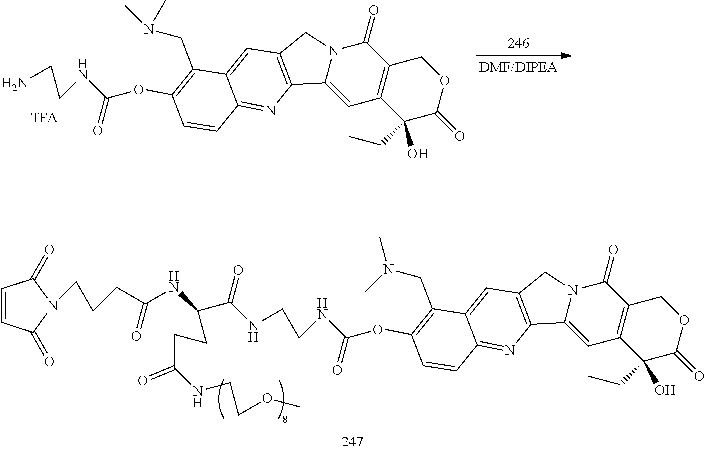

To a solution of compound 243 (18 mg, 0.029 mmol) and compound 246 (24 mg, 0.029 mmol) in DMF (4 mL) at about 0° C., DIPEA (75 mg, 0.58 mmol) was added. The reaction was allowed to warm to room temperature and stirred for 2 hour. After concentration, the residue was purified by HPLC (CH₃CN/H₂O, 20% to 80%) to afford the title compound 247 (15 mg, 45% yield). ESI m/z: [M+H]⁺ calcd for C₅₆H₇₈N₈O₁₉, 1167.54; found, 1167.54.

### Example 96. Synthesis of (S)-tert-butyl 2-((S)-2-aminopropanamido)propanoate (249)

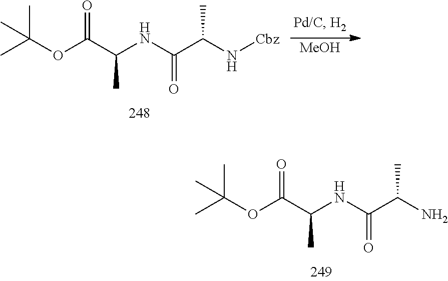 [SMILES: "MeOH" ; C[C@H](NC(=O)OCc1ccccc1)C(=O)N[C@@H](C)C(=O)OC(C)(C)C ; C[C@H](N)C(=O)N[C@@H](C)C(=O)OC(C)(C)C]

A solution of (S)-tert-butyl 2-((S)-2-(((benzyloxy)carbonyl)amino)propanamido)propanoate (10 g, 0.028 mol) and 10% palladium carbon (1.0 g) in methanol (100 mL) was stirred under hydrogen (5 psi) for 3 hours. The solid was filtered off and the filtrated solution was evaporated to give a colorless oil product (6.1 g, 100% yield). ESI m/z: [M+H]⁺ calcd for C₁₀H₂₀N₂O₃, 217.15; found, 217.15.

### Example 97. Synthesis of (30S,33S,36S)-tert-butyl 30-(((benzyloxy)carbonyl)amino)-33,36-dimethyl-27,31,34-trioxo-2,5,8,11,14,17,20,23-octaoxa-26,32,35-triazaheptatriacontan-37-oate (251)

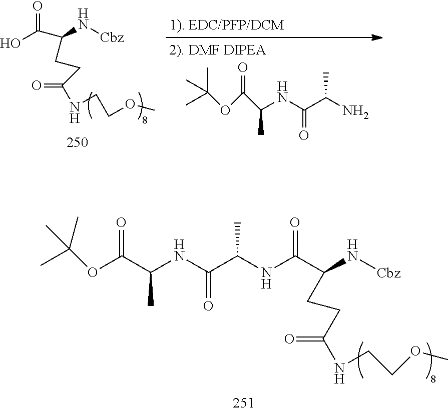

(S)-30-(((benzyloxy)carbonyl)amino)-27-oxo-2,5,8,11,14,17,20,23-octaoxa-26-azahentriacontan-31-oic acid (250) (100 mg, 0.154 mmol) in dichloromethane (5 mL), EDC (59 mg, 0.309 mmol) and pentafluorophenol (PFP) (57 mg, 0.309 mmol) were added. The mixture was stirred at room temperature for 2 hours, diluted with dichloromethane (20 mL), washed with water (5 mL), dried over sodium sulfate, filtered, and concentrated. The residue was re-dissolved in DMF (5 mL), followed by addition of compound 249 (49 mg, 0.23 mmol) and DIPEA (90 mg, 0.69 mmol). The mixture was stirred at room temperature for 1 hour, concentrated, and purified on a short SiO₂ column with elution of MeOH/CH₂Cl₂ (1:10) to give the title compound 251 (80 mg, 61% yield). ESI m/z: [M+H]⁺ calcd for C₄₀H₆N₄O₁₅, 845.47; found, 845.47.

### Example 98. Synthesis of (30S,33S,36S)-tert-butyl 30-amino-33,36-dimethyl-27,31,34-trioxo-2,5,8,11,14,17,20,23-octaoxa-26,32,35-triazaheptatriacontan-37-oate (252)

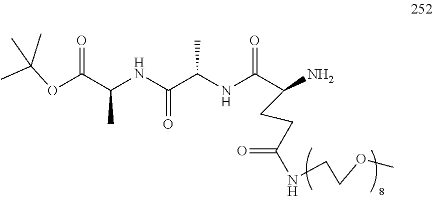

A solution of compound 251 (80 mg, 0.094 mmol) and 10% palladium carbon (10 mg) in methanol (5 mL) was stirred under hydrogen (5 psi) for 2 hours. The solid was filtered off and filtrate was concentrated to give a colorless oil product (252) (66 mg, 100% yield) for next step without further purification. MS-ESI m/z: [M+H]⁺ calcd for C₃₂H₆₂N₄O₁₃, 711.43; found, 711.43.

### Example 99. Synthesis of (30R,33S,36S)-tert-butyl 30-(4-(2,5-dioxo-2,5-dihydro-1H-pyrrol-1-yl)butanamido)-33,36-dimethyl-27,31,34-trioxo-2,5,8,11,14,17,20,23-octaoxa-26,32,35-triazaheptatriacontan-37-oate (253)

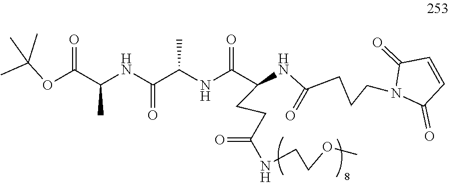

To compound 252 (66 mg, 0.094 mmol) in ethanol (5 mL), 2,5-dioxopyrrolidin-1-yl 4-(2,5-dioxo-2,5-dihydro-1H-pyrrol-1-yl)butanoate (39 mg, 0.141 mmol) and PBS (0.1 M, pH 7.5, 1.0 mL) were added. The reaction mixture was stirred overnight, concentrated and purified on a silica gel column (dichloromethane/MeOH=100:0 to 10:1) to afford the title compound 253 (37 mg, 45% yield). ESI m/z: [M+H]⁺ calcd for C₄₀H₆₉N₅O₁₆, 876.47; found, 876.47.

### Example 100. Synthesis of (30R,33S,36S)-perfluorophenyl 30-(4-(2,5-dioxo-2,5-dihydro-1H-pyrrol-1-yl)butanamido)-33,36-dimethyl-27,31,34-trioxo-2,5,8,11,14,17,20,23-octaoxa-26,32,35-triazaheptatriacontan-37-oate (254)

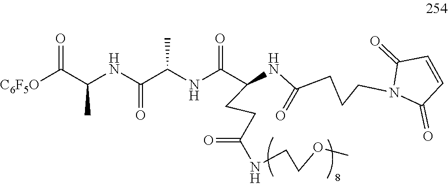

Compound 253 (50 mg, 0.057 mmol) in dichloromethane (3 mL) was treated with TFA (1 mL) at room temperature for 2 hours. The reaction mixture was evaporated to dryness and then re-dissolved in dichloromethane (5 mL), to which EDCI (16 mg, 0.084 mmol) and pentafluorophenol (15 mg, 0.084 mmol) were added. The mixture was stirred at room temperature for 4 hours, concentrated, and purified on a silica gel column (dichloromethane/EtOAc=100:10 to 3:1) to give the title compound 254 (41 mg, 73% yield). ESI m/z: [M+H]⁺ calcd for C₄₂H₆₀F₅N₅O₁₆, 986.40; found, 986.42.

### Example 101. Synthesis of(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl ((30R,33S,36S)-30-(4-(2,5-dioxo-2,5-dihydro-1H-pyrrol-1-yl)butanamido)-33,36-dimethyl-27,31,34,37-tetraoxo-2,5,8,11,14,17,20,23-octaoxa-26,32,35,38-tetraazatetracontan-40-yl)carbamate (255)

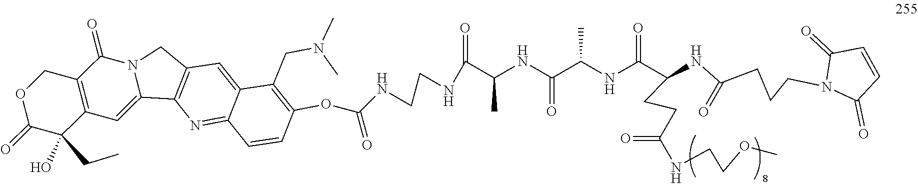

To a solution of compound 243 (25 mg, 0.042 mmol) and compound 254 (41 mg, 0.042 mmol) in DMF (5 mL) at 0° C., DIPEA (80 mg, 0.672 mmol) was added. The reaction mixture was stirred at 0° C. for 1 hour then at room temperature for another 1 hour. After concentration, the residue was purified by prep-HPLC (mobile phase: 10% to 80% acetonitrile/water) to afford the title compound 255 (23 mg, 43% yield). MS-ESI m/z: [M+H]⁺ calcd for C₆₂H₈₈N₁₀O₂₁, 1309.61; found, 1309.65.

### Example 102. Synthesis of di-tert-butyl 4,4′-(((2S,3S)-2,3-bis(((benzyloxy)carbonyl)amino)-succinyl)bis(azanediyl))dibutanoate (256)

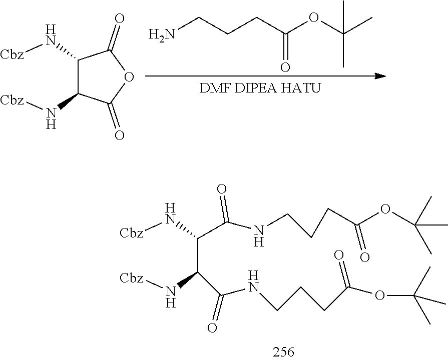 [SMILES: O=C(N[C@@H]1C(=O)OC(=O)[C@H]1NC(=O)OCc1ccccc1)OCc1ccccc1 ; CC(C)(C)OC(=O)CCCN ; CC(C)(C)OC(=O)CCCNC(=O)[C@@H](NC(=O)OCc1ccccc1)[C@H](NC(=O)OCc1ccccc1)C(=O)NCCCC(=O)OC(C)(C)C]

To solution of dibenzyl ((3S,4S)-2,5-dioxotetrahydrofuran-3,4-diyl)dicarbamate (200 mg, 0.5 mmol) in DMF (5 mL) at about 0° C., tert-butyl aminobutyrate (80 mg, 0.5 mmol) was added. The mixture was stirred at 0° C. for 30 min and then room temperature for 30 min. The reaction solution was re-cooled to about 0° C., followed by addition of DIPEA (64 mg, 0.5 mmol), tert-butyl aminobutyrate (80 mg, 0.5 mmol) and HATU (190 mg, 0.5 mmol). The reaction mixture was warmed to room temperature and stirred for 2 hour, then diluted with dichloromethane (50 mL), washed with saturated NaHCO₃ (20 mL), water (10 mL), dried over anhydrous sodium sulfate, filtered and concentrated. The residue was purified by column chromatography (dichloromethane/MeOH=100:0 to 10:1) to give the title compound 256 (262 mg, 75% yield). MS-ESI m/z: [M+H]⁺ calcd for C₃₆H₅₀N₄O₁₀, 699.35; found, 699.35.

### Example 103. Synthesis of di-tert-butyl 4,4′-(((2S,3S)-2,3-diaminosuccinyl)bis(azanediyl))-dibutanoate (257)

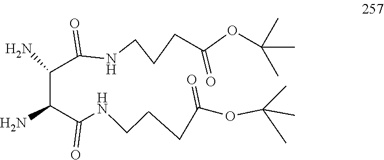 [SMILES: CC(C)(C)OC(=O)CCCNC(=O)[C@@H](N)[C@H](N)C(=O)NCCCC(=O)OC(C)(C)C]

A mixture of compound 256 (100 mg, 0.14 mmol) and 10% palladium carbon (10 mg) in methanol (5 mL) were stirred under hydrogen (5 psi) overnight. The solid was filtered off and filtrated solution was concentrated to give a colorless oil title compound (257) for the next step without purification (61 mg, 100% yield). MS-ESI m/z: [M+H]⁺ calcd for C₂₀H₃₈N₄O₆, 431.28; found, 431.28.

### Example 104. Synthesis of di-tert-butyl 4,4′-(((2S,3S)-2,3-bis(4-(2,5-dioxo-2,5-dihydro-1H-pyrrol-1-yl)butanamido)succinyl)bis(azanediyl))dibutanoate

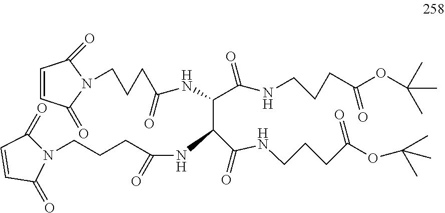 [SMILES: CC(C)(C)OC(=O)CCCNC(=O)[C@@H](NC(=O)CCCN1C(=O)C=CC1=O)[C@H](NC(=O)CCCN1C(=O)C=CC1=O)C(=O)NCCCC(=O)OC(C)(C)C]

To solution of compound 257 (61 mg, 0.14 mmol) in the mixture of ethanol (5 mL) and PBS (0.1 M, pH 7.5, 1.0 mL), 2,5-dioxopyrrolidin-1-yl 4-(2,5-dioxo-2,5-dihydro-1H-pyrrol-1-yl)butanoate (118 mg, 0.42 mmol) was added. The reaction mixture was stirred overnight, concentrated and purified on a silica gel column (dichloromethane/MeOH=100:0 to 10:1) to afford the title compound 258 (65 mg, 60% yield). MS-ESI m/z: [M+H]⁺ calcd for C₃₇H₅₆N₆O₁₂, 777.40; found, 777.41.

### Example 105. Synthesis of 4,4′-(((2S,3S)-2,3-bis(4-(2,5-dioxo-2,5-dihydro-1H-pyrrol-1-yl)butanamido)succinyl)bis(azanediyl))dibutanoic acid (259)

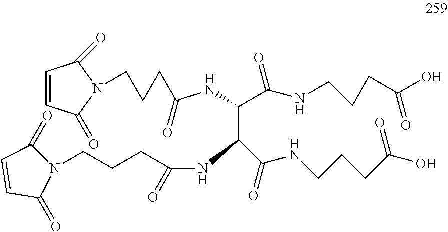 [SMILES: O=C(O)CCCNC(=O)[C@@H](NC(=O)CCCN1C(=O)C=CC1=O)[C@H](NC(=O)CCCN1C(=O)C=CC1=O)C(=O)NCCCC(=O)O]

Compound 258 (65 mg, 0.083 mmol) was dissolved in dichloromethane (6 mL), and treated with trifluoroacetic acid (2 mL) for 2 hours. The reaction mixture was diluted with toluene (5 ml), concentrated to give the title compound 259 (53 mg, 100% yield). MS-ESI m/z: [M+H]⁺ calcd for C₂₈H₃₆N₆O₁₂, 649.24; found, 649.24.

### Example 106. Synthesis of bis((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) ((10S,11S)-10,11-bis(4-(2,5-dioxo-2,5-dihydro-1H-pyrrol-1-yl)butanamido)-4,9,12,17-tetraoxo-3,8,13,18-tetraazaicosane-1,20-diyl)dicarbamate (260)

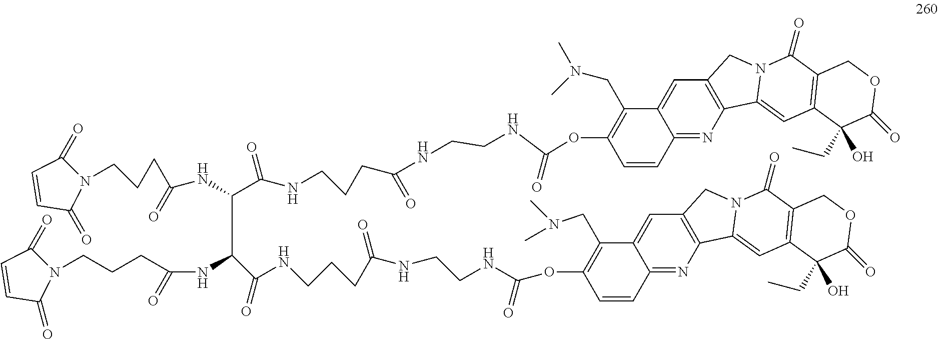 [SMILES: CC[C@@]1(O)C(=O)OCc2c1cc1n(c2=O)Cc2cc3c(CN(C)C)c(OC(=O)NCCNC(=O)CCCNC(=O)[C@@H](NC(=O)CCCN4C(=O)C=CC4=O)[C@H](NC(=O)CCCN4C(=O)C=CC4=O)C(=O)NCCCC(=O)NCCNC(=O)Oc4ccc5nc6c(cc5c4CN(C)C)Cn4c-6cc5c(c4=O)COC(=O)[C@]5(O)CC)ccc3nc2-1]

To a solution of compound 259 (53 mg, 0.083 mmol) in DMF at 0° C., EDC (31 mg, 0.16 mmol), HOBt (22 mg, 0.16 mmol), DIPEA (53 mg, 0.41 mmol) and compound 243 (100 mg, 0.16 mmol) were added. The reaction mixture was allowed to warm to room temperature, stirred for 2 hour, diluted with dichloromethane (50 mL), washed with water (10 mL) and brine (10 mL), dried over sodium sulfate, filtered and concentrated. The residue was purified by prep-HPLC (mobile phase: 10% to 80% CH₃CN/H₂O, with 0.1% formic acid) to afford the title compound 260 (55 mg, 42% yield). ESI m/z: [M+H]⁺ calcd for C₈₀H₉₄N₁₆O₂₀, 1599.68; found, 1599.68.

### Example 107. Synthesis of (2S,5S,8S,9S,12S,15S)-di-tert-butyl 8,9-bis(((benzyloxy)carbonyl)amino)-2,5,12,15-tetramethyl-4,7,10,13-tetraoxo-3,6,11,14-tetraazahexadecane-1,16-dioate (261)

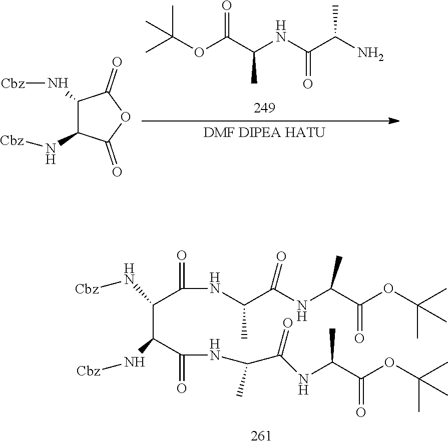

To a solution of compound 249 (200 mg, 0.5 mmol) in DMF (5 mL) at about 0° C., dibenzyl ((3S,4S)-2,5-dioxotetrahydrofuran-3,4-diyl)dicarbamate (216 mg, 1.0 mmol) was added. The mixture was stirred at 0° C. for 30 min, room temperature for 45 min, then re-cooled to about 0° C., followed by addition of DIPEA (64 mg, 0.5 mmol) and EDC (458 mg, 2.41 mmol). The reaction mixture was warmed to room temperature and stirred for 1 hour, then diluted with dichloromethane (50 mL), washed with saturated NaHCO₃ (20 mL), water (10 mL), dried over anhydrous sodium sulfate, filtered and concentrated. The residue was purified by column chromatography (100:0 to 10:1 dichloromethane/MeOH) to give compound 261 (264 mg, 65% yield). MS-ESI m/z: [M+H]⁺ calcd for C₄₀H₅₆N₆O₁₂, 813.40; found, 813.40.

### Example 108. Synthesis of (2S,5S,8S,9S,12S,15S)-di-tert-butyl 8,9-diamino-2,5,12,15-tetramethyl-4,7,10,13-tetraoxo-3,6,11,14-tetraazahexadecane-1,16-dioate (262)

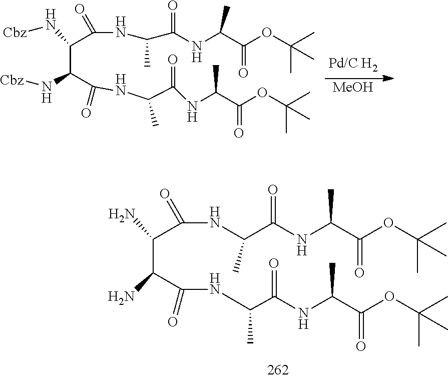 [SMILES: "MeOH" ; C[C@H](NC(=O)[C@@H](NC(=O)OCc1ccccc1)[C@H](NC(=O)OCc1ccccc1)C(=O)N[C@@H](C)C(=O)N[C@@H](C)C(=O)OC(C)(C)C)C(=O)N[C@@H](C)C(=O)OC(C)(C)C ; C[C@H](NC(=O)[C@@H](N)[C@H](N)C(=O)N[C@@H](C)C(=O)N[C@@H](C)C(=O)OC(C)(C)C)C(=O)N[C@@H](C)C(=O)OC(C)(C)C]

A mixture of compound 261 (264 mg, 0.32 mmol) and 10% palladium carbon (10 mg) in methanol (5 mL) was stirred under hydrogen overnight. The solid was filtered off and filtrate concentrated to give a colorless oil (177 mg, 100% yield). ESI m/z: [M+H]⁺ calcd for C₂₄H₄₄N₆O₈, 545.32; found, 545.32.

### Example 109. Synthesis of (2S,5S,8S,9S,12S,15S)-di-tert-butyl 8,9-bis(4-(2,5-dioxo-2,5-dihydro-1H-pyrrol-1-yl)butanamido)-2,5,12,15-tetramethyl-4,7,10,13-tetraoxo-3,6,11,14-tetraazahexadecane-1,16-dioate (263)

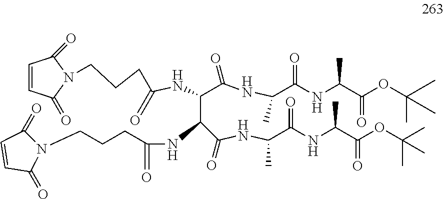 [SMILES: C[C@H](NC(=O)[C@@H](NC(=O)CCCN1C(=O)C=CC1=O)[C@H](NC(=O)CCCN1C(=O)C=CC1=O)C(=O)N[C@@H](C)C(=O)N[C@@H](C)C(=O)OC(C)(C)C)C(=O)N[C@@H](C)C(=O)OC(C)(C)C]

To a solution of compound 262 (177 mg, 0.32 mmol) in the mixture of ethanol (5 mL) and PBS (0.1 M, pH 7.5, 1.0 mL), 2,5-dioxopyrrolidin-1-yl 4-(2,5-dioxo-2,5-dihydro-1H-pyrrol-1-yl)butanoate (136 mg, 0.48 mmol) was added. The reaction mixture was stirred overnight, concentrated and purified on a silica gel column (dichloromethane/MeOH=100:0 to 10:1) to afford the title compound 263 (127 mg, 45% yield). MS-ESI m/z: [M+H]⁺ calcd for C₄₀H₅₈N₈O₁₄, 875.41; found 875.42.

### Example 110. Synthesis of (2S,5S,8S,9S,12S,15S)-8,9-bis(4-(2,5-dioxo-2,5-dihydro-1H-pyrrol-1-yl)butanamido)-2,5,12,15-tetramethyl-4,7,10,13-tetraoxo-3,6,11,14-tetraazahexadecane-1,16-dioic acid (264)

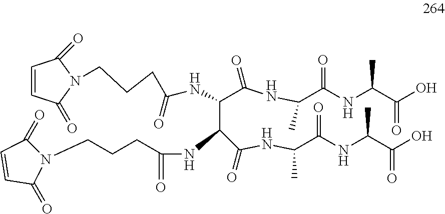 [SMILES: C[C@H](NC(=O)[C@H](C)NC(=O)[C@@H](NC(=O)CCCN1C(=O)C=CC1=O)[C@H](NC(=O)CCCN1C(=O)C=CC1=O)C(=O)N[C@@H](C)C(=O)N[C@@H](C)C(=O)O)C(=O)O]

Compound 263 (127 mg, 0.14 mmol) in dichloromethane (3 mL) was treated with trifluoroacetic acid (3 mL) for 2 hours. The reaction mixture was evaporated to give the product 264 (111 mg, 100% yield). MS-ESI m/z: [M+H]⁺ calcd for C₃₂H₄₂N₈O₁₄, 763.28; found, 763.28.

### Example 111. Synthesis of bis((S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl) ((5S,8S,11S,12S,15S,18S)-11,12-bis(4-(2,5-dioxo-2,5-dihydro-1H-pyrrol-1-yl)butanamido)-5,8,15,18-tetramethyl-4,7,10,13,16,19-hexaoxo-3,6,9,14,17,20-hexaazadocosane-1,22-diyl)dicarbamate (265)

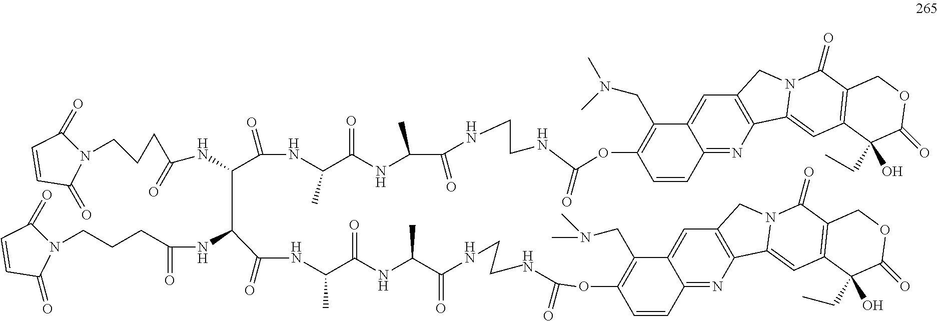

To a solution of compound 264 (61 mg, 0.08 mmol) in DMF (5 mL) at about 0° C., EDC (31 mg, 0.16 mmol), HOBt (22 mg, 0.16 mmol), DIPEA (53 mg, 0.41 mmol) and compound 243 (100 mg, 0.16 mmol) were added. The reaction was warmed to room temperature and stirred for 2 hour, then diluted with dichloromethane (50 mL), washed with saturated water (10 mL), brine (10 mL), dried over anhydrous sodium sulfate, filtered and concentrated. The residue was purified by prep-HPLC (10% to 80% CH₃CN/H₂O, with 0.1% formic acid) to give the title compound 265 (55 mg, 40% yield). MS-ESI m/z: [M+H]⁺ calcd for C₈₄H₉₆N₁₈O₂₄, 1741.68; found, 1741.68.

### Example 112. Synthesis of (2S,3S)-2,3-bis(((benzyloxy)carbonyl)amino)-4-((4-(tert-butoxy)-4-oxobutyl)amino)-4-oxobutanoic acid

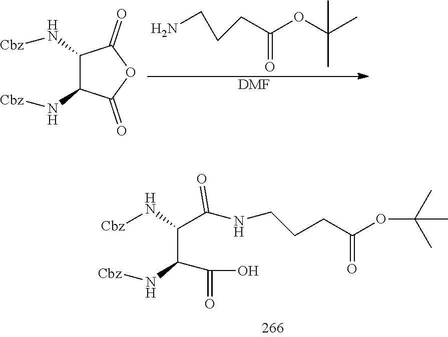

To a solution of tert-butyl aminobutyrate (80 mg, 0.5 mmol) in DMF (5 mL), dibenzyl ((3S,4S)-2,5-dioxotetrahydrofuran-3,4-diyl)dicarbamate (200 mg, 0.5 mmol) was added. The mixture was stirred at room temperature for 3 h. Concentration of the reaction mixture gave a crude product 266 without further purification (280 mg, 100% yield). MS-ESI m/z: [M+H]⁺ calcd for C₂₈H₃₅N₃O₉, 558.24; found, 558.24.

### Example 113. Synthesis of (28S,29S)-tert-butyl 28,29-bis(((benzyloxy)carbonyl)amino)-27,30-dioxo-2,5,8,11,14,17,20,23-octaoxa-26,31-diazapentatriacontan-35-oate (267)

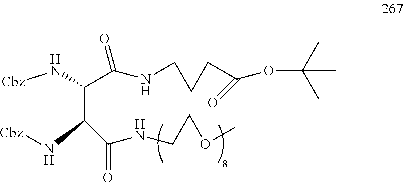

To a solution of compound 266 (280 mg, 0.5 mmol) and compound 31 (229 mg, 0.6 mmol) in DMF (10 mL) at 0° C., HATU (228 mg, 0.6 mmol) and DIPEA (77 mg, 0.6 mmol) were added. The reaction mixture was warmed to room temperature and stirred for 1 hour, diluted with dichloromethane (50 mL), washed with water (10 mL), saturated NaHCO₃ (10 mL), brine (10 mL), dried over anhydrous sodium sulfate, filtered, concentrated and purification on SiO2 column with EtOAc/DCM (1:3) to afford the title compound 267 (392 mg, 85% yield). MS-ESI m/z: [M+H]⁺ calcd for C₄₅H₇₀N₄O₁₆, 923.48; found, 923.48.

### Example 114. Synthesis of (28S,29S)-tert-butyl 28,29-diamino-27,30-dioxo-2,5,8,11,14,17,20,23-octaoxa-26,31-diazapentatriacontan-35-oate (268)

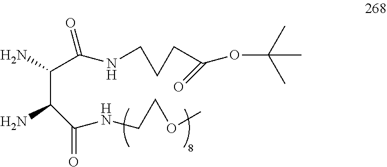

Compound 267 (129 mg, 0.14 mmol) and 10% palladium carbon (10 mg) in methanol (10 mL) was stirred under hydrogen (1.2 atmosphere pressure) overnight. The solid was filtered off and the filtrated solution was evaporated to give a colorless oil product (91 mg, 100% yield) for the next step without further purification. MS-ESI m/z: [M+H]⁺ calcd for C₂₉H₅₈N₄O₁₂, 655.41; found, 655.41.

### Example 115. Synthesis of (28S,29S)-tert-butyl 28,29-bis(4-(2,5-dioxo-2,5-dihydro-1H-pyrrol-1-yl)butanamido)-27,30-dioxo-2,5,8,11,14,17,20,23-octaoxa-26,31-diazapentatriacontan-35-oate (269)

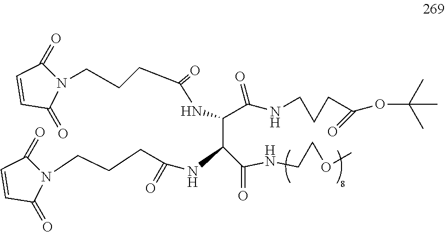

A mixture of compound 268 (91 mg, 0.14 mmol) and 2,5-dioxopyrrolidin-1-yl 4-(2,5-dioxo-2,5-dihydro-1H-pyrrol-1-yl)butanoate (118 mg, 0.42 mmol) in of ethanol (10 mL) PBS (0.1 M, pH 7.5, 3.0 mL) was stirred overnight, concentrated and purified on a silica gel column (dichloromethane/MeOH=100:0 to 10:1) to afford the title compound 269 (71 mg, 50% yield). MS-ESI m/z: [M+H]⁺ calcd for C₄₅H₇₂N₆O₁₈, 985.49; found, 985.49.

### Example 116. Synthesis of (28S,29S)-perfluorophenyl 28,29-bis(4-(2,5-dioxo-2,5-dihydro-1H-pyrrol-1-yl)butanamido)-27,30-dioxo-2,5,8,11,14,17,20,23-octaoxa-26,31-diazapentatriacontan-35-oate (270)

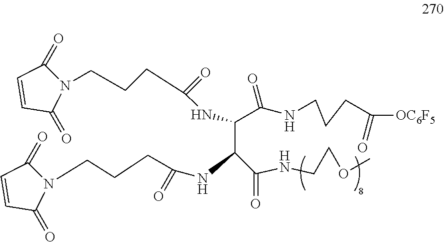

Compound 269 (71 mg, 0.07 mmol) in dichloromethane (5 mL) was treated with TFA (2 mL) at room temperature for 2 hours. The reaction mixture was concentrated and re-dissolved in dichloromethane (5 mL), to which EDCI (54 mg, 0.28 mmol), pentafluorophenol (26 mg, 0.14 mmol) and DIPEA (0.05 mL) were added. The mixture was stirred at room temperature for 4 hours, concentrated and purified on a silica gel column (dichloromethane/EtOAc=10:1 to 10:3) to give the title compound 270 (78 mg, 100% yield). MS-ESI m/z: [M+H]⁺ calcd for C₄₇H₆₃F₅N₆O₁₈, 1095.41; found, 1095.41.

### Example 117. Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl ((28S,29S)-28,29-bis(4-(2,5-dioxo-2,5-dihydro-1H-pyrrol-1-yl)butanamido)-27,30,35-trioxo-2,5,8,11,14,17,20,23-octaoxa-26,31,36-triazaoctatriacontan-38-yl)carbamate (271)

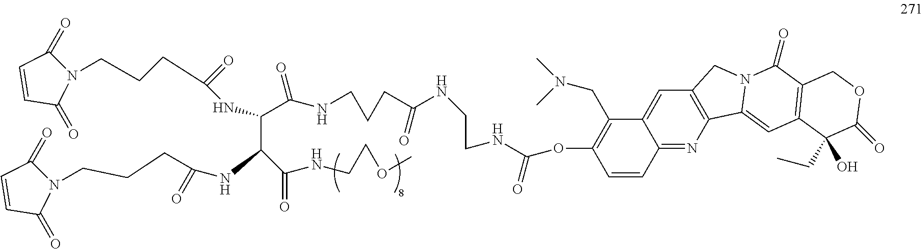

A mixture of compound 243 (42 mg, 0.07 mmol) and compound 270 (78 mg, 0.07 mmol) in DMF (5 mL) at 0° C., DIPEA (18 mg, 0.14 mmol) was added. The reaction mixture was allowed to warm to room temperature and stirred for 1 hour. After concentration, the residue was purified by prep-HPLC (mobile phase: 10% to 80% acetonitrile/water) to afford compound 271 (41 mg, 40% yield). MS-ESI m/z: [M+H]⁺ calcd for C₆₇H₉₁N₁₁O₂₃, 1418.63; found, 1418.63.

### Example 118. Synthesis of perfluorophenyl 4-(bis(2-(2,5-dioxo-2,5-dihydro-1H-pyrrol-1-yl)ethyl)amino)-4-oxobutanoate (272)

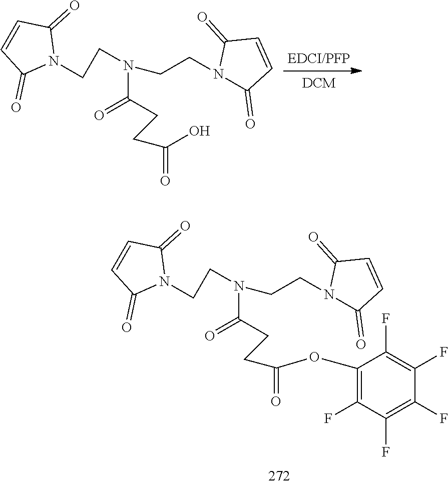 [SMILES: O=C(O)CCC(=O)N(CCN1C(=O)C=CC1=O)CCN1C(=O)C=CC1=O ; O=C(CCC(=O)N(CCN1C(=O)C=CC1=O)CCN1C(=O)C=CC1=O)Oc1c(F)c(F)c(F)c(F)c1F]

To a solution of 4-(bis(2-(2,5-dioxo-2,5-dihydro-1H-pyrrol-1-yl)ethyl)amino)-4-oxobutanoic acid (100 mg, 0.27 mmol) in dichloromethane (5 mL), EDC (210 mg, 1.10 mmol) and pentafluorophenol (101 mg, 0.55 mmol) were added. The mixture was stirred at room temperature for 3 hour, concentrated and purified on a silica gel column (dichloromethane/EtOAc=20:1 to 5:1) to give the title compound 272 (114 mg, 80% yield). MS-ESI m/z: [M+H]⁺ calcd for C₂₂H₁₆F₅N₃O₇, 530.09; found, 530.09.

### Example 119. Synthesis of (S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl (2-(4-(bis(2-(2,5-dioxo-2,5-dihydro-1H-pyrrol-1-yl)ethyl)amino)-4-oxobutanamido)ethyl)carbamate (273)

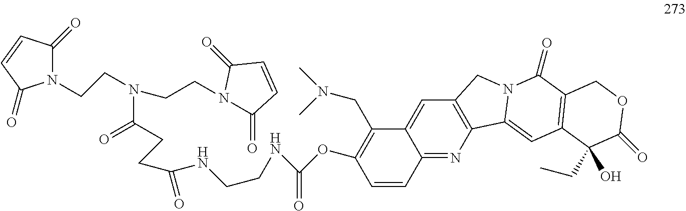 [SMILES: CC[C@@]1(O)C(=O)OCc2c1cc1n(c2=O)Cc2cc3c(CN(C)C)c(OC(=O)NCCNC(=O)CCC(=O)N(CCN4C(=O)C=CC4=O)CCN4C(=O)C=CC4=O)ccc3nc2-1]

To a mixture of compound 243 (20 mg, 0.033 mmol) and compound 272 (17 mg, 0.033 mmol) in DMF (5 mL) at 0° C., DIPEA (8.5 mg, 0.066 mmol) was added. The reaction mixture was stirred at 0° C. for 0.5 h, followed by at room temperature for 2 h. Then the reaction mixture was concentrated and purified by prep-HPLC (mobile phase: acetonitrile/water=10% to 80% with 0.1% formic acid) to afford the title compound 273 (12.6 mg, 45% yield). MS-ESI m/z: [M+H]⁺ calcd for C₄₂H₄₄N₈O₁₂, 853.31; found, 853.31.

### Example 120. Synthesis of (S)-2-((S)-2-(4-(bis(2-(2,5-dioxo-2,5-dihydro-1H-pyrrol-1-yl)ethyl)amino)-4-oxobutanamido)propanamido)propanoic acid (274)

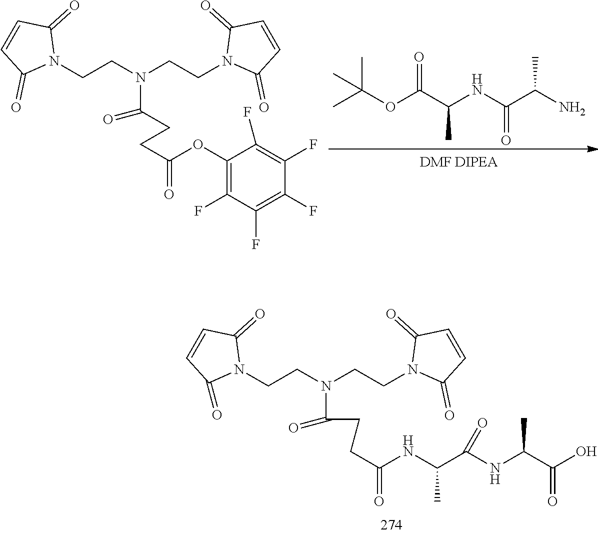

To a mixture of (S)-2-((S)-2-aminopropanamido)propanoic acid (20 mg, 0.094 mmol) and compound 272 (50 mg, 0.094 mmol) in DMF (5 mL) at 0° C., DIPEA (240 mg, 1.90 mmol) was added. The reaction mixture was stirred at 0° C. for 0.5 h, followed by at room temperature for 2 h. Then the reaction mixture was concentrated and purified on a short silica gel column (dichloromethane/CH₃OH=10:1 to 5:2) to give the title compound 274 (12.6 mg, 45% yield). MS-ESI m/z: [M+H]⁺ calcd for C₂₆H₃₅N₅O₉, 562.24; found, 562.24.

### Example 121. Synthesis of (S)-perfluorophenyl 2-((S)-2-(4-(bis(2-(2,5-dioxo-2,5-dihydro-1H-pyrrol-1-yl)ethyl)amino)-4-oxobutanamido)propanamido)propanoate (275)

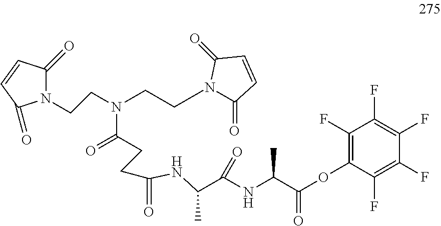

To a solution of compound 274 (47 mg, 0.084 mmol) in dichloromethane (5 mL), EDC (210 mg, 1.10 mmol) and pentafluorophenol (50.0 mg, 0.27 mmol) were added. The mixture was stirred at room temperature for 3 hour, concentrated and purified on a silica gel column (dichloromethane/EtOAc=20:1 to 5:1) to give the title compound 275 (44.6 mg, 79% yield). MS-ESI m/z: [M+H]⁺ calcd for C₂₈H₂₇F5N₅O9, 672.17; found, 672.17.

### Example 122. Synthesis of(S)-10-((dimethylamino)methyl)-4-ethyl-4-hydroxy-3,14-dioxo-3,4,12,14-tetrahydro-1H-pyrano[3′,4′:6,7]indolizino[1,2-b]quinolin-9-yl ((5S,8S)-16-(2,5-dioxo-2,5-dihydro-1H-pyrrol-1-yl)-14-(2-(2,5-dioxo-2,5-dihydro-1H-pyrrol-1-yl)ethyl)-5,8-dimethyl-4,7,10,13-tetraoxo-3,6,9,14-tetraazahexadecyl)carbamate (276)

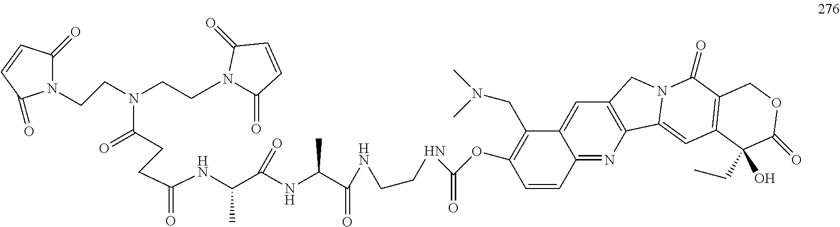

To a solution of compound 243 (40 mg, 0.065 mmol) and compound 275 (43.6 mg, 0.065 mmol) in DMF (5 mL) at 0° C., DIPEA (240 mg, 1.90 mmol) was added. The reaction mixture was stirred at 0° C. for 0.5 h, followed by at room temperature for 2 h. Then the reaction mixture was concentrated and purified by prep-HPLC (mobile phase: acetonitrile/water=10% to 80% with 0.1% formic acid) to afford compound 276 (32 mg, 50% yield). MS-ESI m/z: [M+H]⁺ calcd for C₄₈H₅₄N₁₀O₁₄, 995.38; found, 995.38.

### Example 123. Synthesis of(S)-1-(9-(((2-(tert-butoxy)-2-oxoethyl)carbamoyl)oxy)-4-ethyl-4-hydroxy-3,14-dioxo-3,4,12,14-tetrahydro-1H-pyrano[3′,4′:6,7]indolizino[1,2-b]quinolin-10-yl)-N,N-dimethyl-N-(4-nitrobenzyl)methanaminium (277)

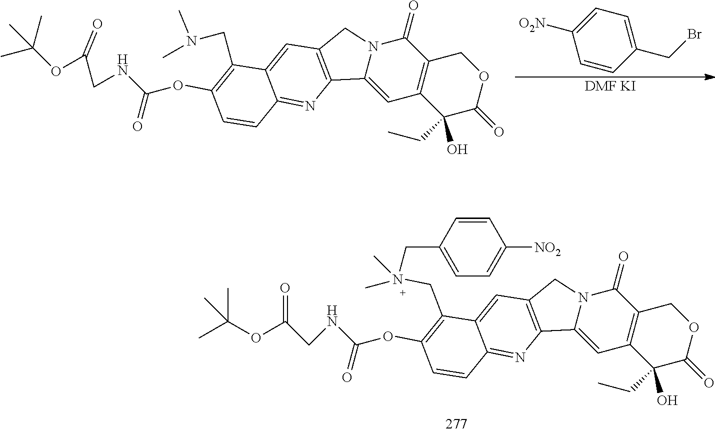

To a solution of compound 242 (50 mg, 0.069 mmol) in DMF (3 mL), p-nitrobenzyl bromide (32 mg, 0.138 mmol) and a catalytic amount of potassium iodide (2 mg) were added. The reaction mixture was heated to 60° C. for 4 hours and concentrated, triturated with ethyl acetate to give a yellow solid (25 mg, 50% yield). MS-ESI m/z: [M+H]⁺ calcd for C₃₇H₄₀N₅O₁₀, 714.28; found, 714.28.

### Example 124. Synthesis of (S)—N-(4-aminobenzyl)-1-(9-(((2-(tert-butoxy)-2-oxoethyl)carbamoyl)oxy)-4-ethyl-4-hydroxy-3,14-dioxo-3,4,12,14-tetrahydro-1H-pyrano[3′,4′:6,7]indolizino[1,2-b]quinolin-10-yl)-N,N-dimethylmethanaminium (278)

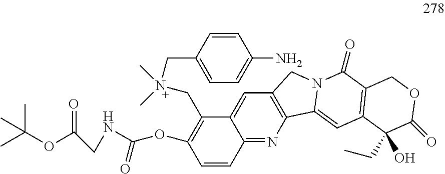 [SMILES: CC[C@@]1(O)C(=O)OCc2c1cc1n(c2=O)Cc2cc3c(C[N+](C)(C)Cc4ccc(N)cc4)c(OC(=O)NCC(=O)OC(C)(C)C)ccc3nc2-1]

A solution of compound 277 (100 mg, 0.14 mmol), hydrazine hydrate (7 mg, 0.14 mmol) and FeCl₃ (324 mg, 0.14 mmol) in ethanol (15 mL) were refluxed for 2 hours until the reaction was completed. After concentration, the residue was triturated with ethyl acetate to give a yellow solid product (81 mg, 85% yield). MS-ESI m/z: [M+H]⁺ calcd for C₃₇H₄₂N₅O₈, 684.30; found, 684.30.

### Example 125. Synthesis of 1-((S)-9-(((carboxymethyl)carbamoyl)oxy)-4-ethyl-4-hydroxy-3,14-dioxo-3,4,12,14-tetrahydro-1H-pyrano[3′,4′:6,7]indolizino[1,2-b]quinolin-10-yl)-N-(4-((30S,33S,36S)-30-(4-(2,5-dioxo-2,5-dihydro-1H-pyrrol-1-yl)butanamido)-33,36-dimethyl-27,31,34-trioxo-2,5,8,11,14,17,20,23-octaoxa-26,32,35-triazaheptatriacontanamido)benzyl)-N,N-dimethylmethanaminium (279)

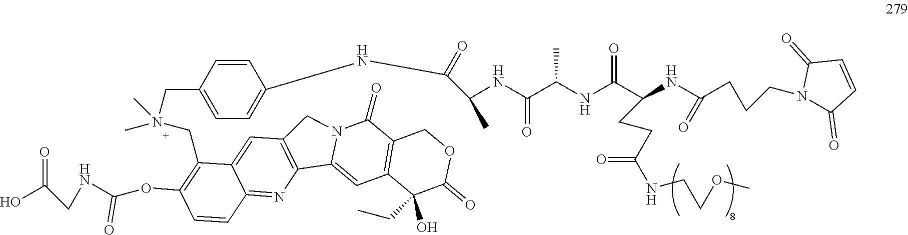

A solution of compound 278 (20 mg, 0.029 mmol) and compound 254 (34 mg, 0.034 mmol) in DMF (5 mL) at 0° C., DIPEA (75 mg, 0.58 mmol) was added. The reaction mixture was stirred at 0° C. for 0.5 h, followed by at room temperature for 2 h. Then the reaction mixture was concentrated, and re-dissolved in the mixture of dichloromethane (3 mL) and TFA (1 mL). After stirring for 1 hour, the reaction mixture was concentrated and purified by prep-HPLC (mobile phase: acetonitrile/water=10% to 80% with 0.1% formic acid) to afford the title compound 279 (12 mg, 30% yield). MS-ESI m/z: [M+H]⁺ calcd for C₆₉H₉₃N₁₀O₂₃, 1429.64; found, 1429.80.

### Example 126. Synthesis of N-(4-((28S,29S)-28,29-bis(4-(2,5-dioxo-2,5-dihydro-1H-pyrrol-1-yl)butanamido)-27,30-dioxo-2,5,8,11,14,17,20,23-octaoxa-26,31-diazapentatriacontanamido)-benzyl)-1-((S)-9-(((carboxymethyl)carbamoyl)oxy)-4-ethyl-4-hydroxy-3,14-dioxo-3,4,12,14-tetrahydro-1H-pyrano[3′,4′:6,7]indolizino[1,2-b]quinolin-10-yl)-N,N-dimethylmethanaminium (280)

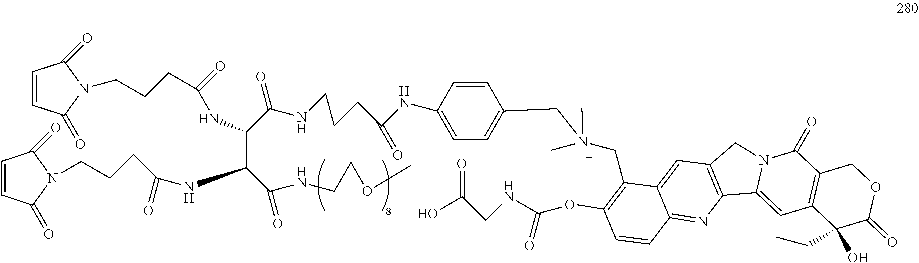

A solution of compound 278 (20 mg, 0.029 mmol) and compound 270 (37 mg, 0.034 mmol) in DMF (5 mL) at 0° C., DIPEA (75 mg, 0.58 mmol) was added. The reaction mixture was stirred at 0° C. for 0.5 h, followed by at room temperature for 2 h. Then the reaction mixture was concentrated, and re-dissolved in the mixture of dichloromethane (3 mL) and TFA (1 mL). After stirring for 0.5 hour, the reaction mixture was diluted with toluene (5 mL), concentrated and purified by prep-HPLC (mobile phase: acetonitrile/water=10% to 80% with 0.1% formic acid) to afford the title compound 280 (17 mg, 40% yield). MS-ESI m/z: [M+H]⁺ calcd for C₇₄H₉₆N₁₁O₂₅, 1538.66; found, 1538.66.

### Example 127. Synthesis of N-(4-((S)-2-((S)-2-(4-(bis(2-(2,5-dioxo-2,5-dihydro-1H-pyrrol-1-yl)ethyl)amino)-4-oxobutanamido)propanamido)propanamido)benzyl)-1-((S)-9-(((carboxymethyl)carbamoyl)oxy)-4-ethyl-4-hydroxy-3,14-dioxo-3,4,12,14-tetrahydro-1H-pyrano[3′,4′:6,7]indolizino[1,2-b]quinolin-10-yl)-N,N-dimethylmethanaminium (281)

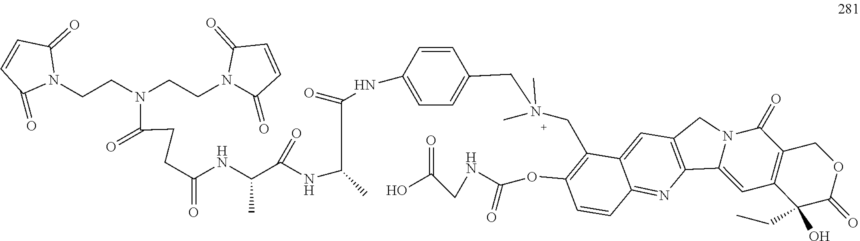

A solution of compound 278 (20 mg, 0.029 mmol) and compound 275 (23 mg, 0.034 mmol) in DMF (5 mL) at 0° C., DIPEA (75 mg, 0.58 mmol) was added. The reaction mixture was stirred at 0° C. for 0.5 h, followed by at room temperature for 2 h. Then the reaction mixture was concentrated, and re-dissolved in the mixture of dichloromethane (3 mL) and TFA (1 mL). After stirring for 0.5 h, the reaction mixture was diluted with toluene (5 mL), concentrated and purified by prep-HPLC (mobile phase: acetonitrile/water=10% to 80% with 0.1% formic acid) to afford the title compound 281 (12 mg, 35% yield). MS-ESI m/z: [M+H]⁺ calcd for C₅₅H₅₉N₁₀O₁₆, 1115.41; found, 1115.47.

### Example 128. Synthesis of 2-(1,3-dioxoisoindolin-2-yl)acetyl chloride (282)

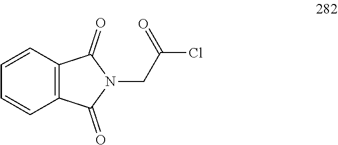 [SMILES: O=C(Cl)CN1C(=O)c2ccccc2C1=O]

To a solution of N-Phthaloylglycine (10.0 g, 48.7 mmol) in dichloromethane (100 mL) oxalyl chloride (6.3 mL, 73.1 mmol) was added, followed by addition of a drop of DMF. The reaction was stirred for 2 h and then concentrated to give compound 282 (10.8 g) as a yellow solid.

### Example 129. Synthesis of tert-butyl 2-(2-(1,3-dioxoisoindolin-2-yl)acetyl)hydrazinecarboxylate (283)

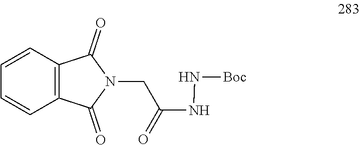 [SMILES: CC(C)(C)OC(=O)NNC(=O)CN1C(=O)c2ccccc2C1=O]

To a solution of Boc-hydrazine (7.08. g, 53.5 mmol) in dichloromethane (200 mL) at 0° C., Et₃N (13.5 mL, 97.4 mmol) and compound 282 (10.8 g, 48.7 mmol) was added in sequence. After stirred at r.t. for 30 min, the mixture was poured into ice-water (100 mL) and extracted with dichloromethane (3×100 mL). The combined organic phases were washed with water (100 mL) and brine (100 mL), dried over anhydrous sodium sulfate, filtered and concentrated to give a white solid product (15.5 g, 100% yield). ESI MS m/z 320.12 ([M+H]⁺).

### Example 130. Synthesis of 2-(1,3-dioxoisoindolin-2-yl)acetohydrazide (284)

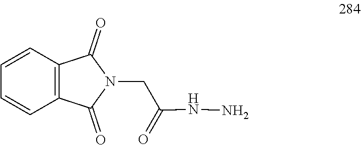 [SMILES: NNC(=O)CN1C(=O)c2ccccc2C1=O]

Compound 283 (15.5 g, 48.7 mmol) was dissolved in dichloromethane (150 mL) and treated with TFA (50 mL) at r.t. for 1 h, then evaporated in vacuo to give a white solid (10.6 g, 100% yield). ESI MS m/z 220.06 ([M+H]⁺).

### Example 131. Synthesis of 2-(1,3-dioxoisoindolin-2-yl)-N′-(2-(1,3-dioxoisoindolin-2-yl)acetyl)acetohydrazide (285)

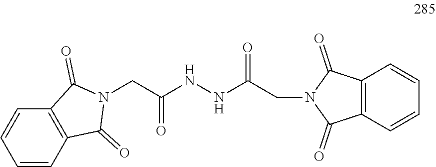 [SMILES: O=C(CN1C(=O)c2ccccc2C1=O)NNC(=O)CN1C(=O)c2ccccc2C1=O]

To a solution of compound 284 (10.6 g, 48.7 mmol) in dichloromethane (200 mL) at 0° C., Et₃N (13.5 mL, 97.4 mmol) and compound 282 (10.8 g, 48.7 mmol) were added. The reaction was warmed to r.t. and stirred overnight. The precipitate was collected by filtration and suspended in water (100 mL) and stirred for 20 min. The mixture was filtered again and a white solid (15.7 g, 80% yield) was collected. ESI MS m/z 407.09 ([M+H]⁺).

### Example 132. Synthesis of di-tert-butyl 2,2′-(1,2-bis(2-(1,3-dioxoisoindolin-2-yl)acetyl)hydrazine-1,2-diyl)diacetate (286)

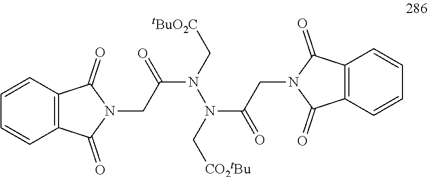 [SMILES: CC(C)(C)OC(=O)CN(C(=O)CN1C(=O)c2ccccc2C1=O)N(CC(=O)OC(C)(C)C)C(=O)CN1C(=O)c2ccccc2C1=O]

NaH (0.5 g, 12.3 mmol) was added to a solution of compound 285 (2.0 g, 4.92 mmol) in DMF (40 mL) at 0° C. in portions. The mixture was warmed to r.t. and stirred for 3 h. After that tert-butyl bromoacetate (2.0 g, 10.3 mmol) was added and the reaction was stirred overnight before pouring into ice-water (100 mL) and extracting with dichloromethane (3×50 mL). The combined organic phase was washed with water (50 mL), brine (50 mL), dried over anhydrous sodium sulfate, filtered and concentrated, purified by silica gel chromatography to give a white solid (1.5 g, 50% yield). ESI MS m/z 635.23 ([M+H]⁺).

### Example 133. Synthesis of di-tert-butyl 2,2′-(1,2-bis(2-aminoacetyl)hydrazine-1,2-diyl)diacetate (287)

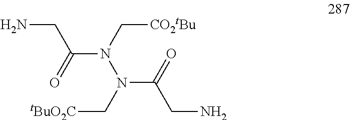 [SMILES: CC(C)(C)OC(=O)CN(C(=O)CN)N(CC(=O)OC(C)(C)C)C(=O)CN]

A mixture of compound 286 (1.5 g, 2.36 mmol) and hydrazine (442 mg, 7.08 mmol) in ethanol (30 mL) was refluxed for 1 h, then cooled to r.t. and filtered. The filtrate was concentrated and taken up in ethyl acetate (20 mL), filtered again. The filtrate was concentrated to give a white solid 287 (750 mg, 85% yield). ESI MS m/z 375.22 ([M+H]⁺).

### Example 134. Synthesis of di-tert-butyl 2,2′-(1,2-bis(2-(2,5-dioxo-2,5-dihydro-1H-pyrrol-1-yl)acetyl)hydrazine-1,2-diyl)diacetate (288)

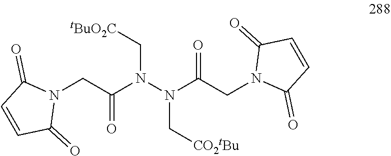 [SMILES: CC(C)(C)OC(=O)CN(C(=O)CN1C(=O)C=CC1=O)N(CC(=O)OC(C)(C)C)C(=O)CN1C(=O)C=CC1=O]

A solution of compound 287 (750 mg, 2 mmol) in THF (2 mL) and saturated NaHCO₃ aqueous solution (30 mL) at 0° C., N-methoxycarbonyl maleimide (622 mg, 4 mmol) was added. The reaction mixture was stirred at 0° C. for 1 h. A white solid was collected by filtration (854 mg, 80% yield). ESI MS m/z 535.20 ([M+H]⁺).

### Example 135. Synthesis of 2,2′-(1,2-bis(2-(2,5-dioxo-2,5-dihydro-1H-pyrrol-1-yl)acetyl)hydrazine-1,2-diyl)diacetic acid (289)

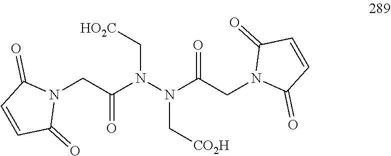 [SMILES: O=C(O)CN(C(=O)CN1C(=O)C=CC1=O)N(CC(=O)O)C(=O)CN1C(=O)C=CC1=O]

Compound 288 (854 mg, 1.6 mmol) was dissolved in dichloromethane (3 mL) and treated with TFA (3 mL) at r.t. for 2 h. The reaction was then evaporated to give compound 289 (675 mg, 100% yield). ESI MS m/z 423.07 ([M+H]⁺).

### Example 136. Synthesis of di-tert-butyl 4,4′-((2,2′-(1,2-bis(2-(2,5-dioxo-2,5-dihydro-1H-pyrrol-1-yl)acetyl)hydrazine-1,2-diyl)bis(acetyl))bis(azanediyl))dibutanoate (290)

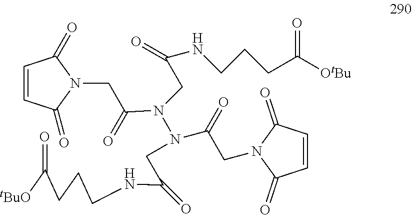 [SMILES: CC(C)(C)OC(=O)CCCNC(=O)CN(C(=O)CN1C(=O)C=CC1=O)N(CC(=O)NCCCC(=O)OC(C)(C)C)C(=O)CN1C(=O)C=CC1=O]

To a solution of compound 289 (200 mg, 0.47 mmol) in DMF (5 mL) at 0° C., tert-butyl 4-aminobutanoate (158 mg, 0.99 mmol) and EDC (189.7 mg, 0.99 mmol) were added. The reaction mixture was warmed to r.t. and stirred overnight, poured into ice-water, and extraction with dichloromethane (3×10 mL). The combined organic phase was washed with 1 N HCl (5 mL), water (5 mL), brine (5 mL), dried over anhydrous sodium sulfate, filtered and concentrated to give a white solid 290 (330 mg, 100% yield).

### Example 137. Synthesis of bis(2,5-dioxopyrrolidin-1-yl) 4,4′-((2,2′-(1,2-bis(2-(2,5-dioxo-2,5-dihydro-1H-pyrrol-1-yl)acetyl)hydrazine-1,2-diyl)bis(acetyl))bis(azanediyl))dibutanoate (291)

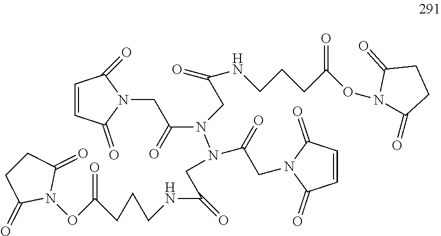 [SMILES: O=C(CN(C(=O)CN1C(=O)C=CC1=O)N(CC(=O)NCCCC(=O)ON1C(=O)CCC1=O)C(=O)CN1C(=O)C=CC1=O)NCCCC(=O)ON1C(=O)CCC1=O]

Compound 290 (330 mg, 0.47 mmol) was dissolved in dichloromethane (3 mL) and treated with TFA (3 mL) at r.t. for 2 h. The reaction was concentrated and re-dissolved in DMF (5 mL) and cooled to 0° C., NHS (113 mg, 0.98 mmol) and EDC (189 mg, 0.98 mmol) were added in sequence. The reaction was warmed to r.t. and stirred overnight, poured into ice-water, and extraction with dichloromethane (3×20 mL). The combined organic phase was washed with water (5 mL), brine (5 mL), dried over anhydrous sodium sulfate, filtered and concentrated to give a white solid 291 (369 mg, 100% yield). ESI MS m/z 787.21 ([M+H]⁺).

### Example 138. Synthesis of(S)-48-(((benzyloxy)carbonyl)amino)-3,16,29,42-tetraoxo-1-phenyl-2,20,23,26,33,36,39-heptaoxa-17,30,43-triazanonatetracontan-49-oic acid (292)

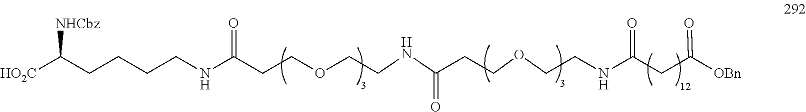

To a solution of compound 235 (1.00 g, 1.32 mmol) in dichloromethane (10 mL) at 0° C., HATU (0.50 g, 1.32 mmol) and TEA (0.06 mL, 1.32 mmol) were added. The reaction was stirred at 0° C. for 30 min, then Z-Lys-OH (0.40 g, 1.43 mmol) was added. The reaction was then stirred at r.t. for 1 h, then diluted with water (20 mL) and extracted with ethyl acetate (3×20 mL). The combined organic phase was washed with brine (30 mL), dried over anhydrous sodium sulfate, filtered and concentrated. The residue was purified by silica gel column (0-10% methanol/dichloromethane) to give a colorless oil 292 (1.28 g, 95% yield). ESI MS m/z 1017.60 ([M+H]⁺).

### Example 139. Synthesis of (S)-47-benzyl 1-(2,5-dioxopyrrolidin-1-yl) 2-(((benzyloxy)carbonyl)amino)-8,21,34-trioxo-11,14,17,24,27,30-hexaoxa-7,20,33-triazaheptatetracontane-1,47-dioate (293)

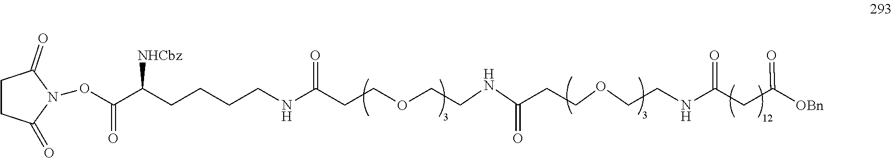

To a solution of compound 292 (1.28 g, 1.26 mmol) in dichloromethane (10 mL), NHS (0.17 g, 1.51 mmol) and EDC⋅HCl (0.29 g, 1.51 mmol) were added, followed by addition of TEA (0.38 mL, 2.77 mmol). The reaction was stirred at r.t. for 2 h, then diluted with water (20 mL) and extracted with ethyl acetate (3×15 mL). The combined organic phase was washed with brine (30 mL), dried over anhydrous sodium sulfate, filtered and concentrated. The residue was purified by silica gel column (0-10% methanol/dichloromethane) to give a colorless oil 293 (1.28 g, 91% yield). ESI MS m/z 1114.62 ([M+H]⁺).

### Example 140. Synthesis of di-tert-butyl 1,2-bis(2-(tert-butoxy)-2-oxoethyl)hydrazine-1,2-dicarboxylate (294)

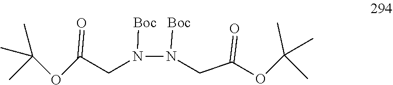 [SMILES: CC(C)(C)OC(=O)CN(C(=O)OC(C)(C)C)N(CC(=O)OC(C)(C)C)C(=O)OC(C)(C)C]

To a solution of di-tert-butyl hydrazine-1,2-dicarboxylate (8.01 g, 34.4 mmol) in DMF (150 ml), NaH (60% in oil, 2.76 g, 68.8 mmol) was added. After stirred at RT for 30 min, tert-butyl 2-bromoacetate (14.01 g, 72.1 mmol) was added. The mixture was stirred overnight, quenched with addition of methanol (3 ml), concentrated, diluted with EtOAc (100 ml) and water (100 ml), separated, and the aqueous layer was extracted with EtOAc (2×50 ml). The organic layers were combined, dried over MgSO₄, filtered, evaporated, and purified purified by SiO₂ column chromatography (EtOAc/Hexane1:5 to 1:3) to afforded the title compound (12.98 g, 82% yield) as a colorless oil. MS ESI m/z calcd for C₂₂H₄₁N₂O₈ [M+H]⁺ 461.28, found 461.40.

### Example 141. Synthesis of 2,2′-(hydrazine-1,2-diyl)diacetic acid (295)

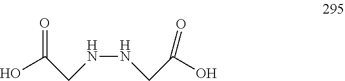

To a solution of di-tert-butyl 1,2-bis(2-(tert-butoxy)-2-oxoethyl)hydrazine-1,2-dicarboxylate (6.51 g, 14.14 mmol) in 1,4-dioxane (40 ml), HCl (12 M, 10 ml) was added. The mixture was stirred for 30 min, diluted with dioxane (20 ml) and toluene (40 ml), evaporated and co-evaporated with dioxane (20 ml) and toluene (40 ml) to dryness to afford the crude title product for the next step without further production (2.15 g, 103% yield, ˜93% pure). MS ESI m/z calcd for C₄H₉N₂O₄[M+H]⁺ 149.05, found 149.40.

### Example 142. Synthesis of 2,2′-(1,2-bis((E)-3-bromoacryloyl)hydrazine-1,2-diyl)diacetic acid (296)

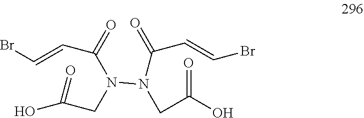 [SMILES: O=C(O)CN(C(=O)/C=C/Br)N(CC(=O)O)C(=O)/C=C/Br]

To a solution of 2,2′-(hydrazine-1,2-diyl)diacetic acid (1.10 g, 7.43 mmol) in the mixture of THF (50 ml) and NaH₂PO₄ (0.1 M, 80 ml, pH 6.0), (E)-3-bromoacryloyl bromide (5.01 g, 23.60 mmol) was added. The mixture was stirred for 6 h, concentrated and purified on SiO₂ column eluted with H₂O/CH₃CN (1:9) containing 3% formic acid to afford the title compound (2.35 g, 77% yield, ˜93% pure). MS ESI m/z calcd for C₁₀H₁₁Br₂N₂O₆ [M+H]⁺ 412.89, found 413.50.

### Example 143. Synthesis of 2,2′-(1,2-bis((E)-3-bromoacryloyl)hydrazine-1,2-diyl)diacetyl chloride (297)

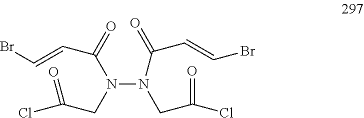 [SMILES: O=C(Cl)CN(C(=O)/C=C/Br)N(CC(=O)Cl)C(=O)/C=C/Br]

To a solution of 2,2′-(1,2-Bis((E)-3-bromoacryloyl)hydrazine-1,2-diyl)diacetic acid (210 mg, 0.509 mmol) in dichloroethane (15 ml), (COCl)₂ (505 mg, 4.01 mmol) was added, followed by addition of 0.040 ml of DMF. After stirred at RT for 2 h, the mixture was concentrated and co-evaporated with dichloroethane (2×20 ml) and toluene (2×15 ml) to dryness to afford the title crude product (which is not stable) for the next step without further purification (245 mg, 107% yield). MS ESI m/z calcd for C₁₀H₉Br₂Cl₂N₂O₄ [M+H]⁺ 448.82, 450.82, 452.82, 454.82, found 448.60, 450.60, 452.60, 454.60.

### Example 144. Synthesis of tert-butyl 2,8-dioxo-1,5-oxazocane-5-carboxylate (299)

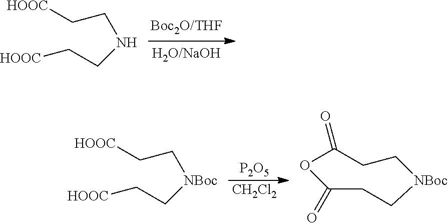

To a solution of 3,3′-azanediyldipropanoic acid (10.00 g, 62.08 mmol) in 1.0 M NaOH (300 ml) at 4° C., di-tert-butyl dicarbonate (22.10 g, 101.3 mmol) in 200 ml THF was added in 1 h. After addition, the mixture was kept to stirring for 2 h at 4° C. The mixture was carefully acidified to pH ˜4 with 0.2 M H₃PO₄, concentrated in vacuo, extracted with CH₂Cl₂, dried over Na₂SO₄, evaporated and purified with flash SiO₂ chromatography eluted with AcOH/MeOH/CH₂Cl₂ (0.01:1:5) to afford 3,3′-((tert-butoxycarbonyl)azanediyl)dipropanoic acid 298 (13.62 g, 84% yield). ESI MS m/z C₁₁H₁₉NO₆ [M+H]⁺, cacld. 262.27, found 262.40.

To a solution of 3,3′-((tert-butoxycarbonyl)azanediyl)dipropanoic acid (8.0 g, 30.6 mmol) in CH₂Cl₂ (500 ml) at 0° C., phosphorus pentoxide (8.70 g, 61.30 mmol) was added. The mixture was stirred at 0° C. for 2 h and then r.t. for 1 h, filtered through short SiO₂ column, and rinsed the column with EtOAc/CH₂Cl₂ (1:6). The filtrate was concentrated and triturated with EtOAc/hexane to afford the title compound 299 (5.64 g, 74% yield). ESI MS m/z C₁₁H₁₇NO₅ [M+H]⁺, cacld. 244.11, found 244.30.

### Example 145. Synthesis of 4-(2,5-dioxo-2,5-dihydro-1H-pyrrol-1-yl)butanoic acid (300)

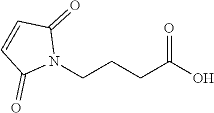 [SMILES: O=C(O)CCCN1C(=O)C=CC1=O]

To a solution of maleic anhydride (268 g, 2.73 mol) in acetic acid (1 L), 4-aminobutanoic acid (285 g, 2.76 mol) was added. After stirring at r.t. for 30 min, the reaction was refluxed for 1.5 h, cooled to r.t. and evaporated under vacuum to give a residue, which was taken up in EA, washed with water and brine, and dried over anhydrous Na₂SO₄, filtered and concentrated. The crude product was crystallized from EtOAc and PE to give a white solid (400 g, 80% yield). 1H NMR (500 MHz, CDCl3) δ 6.71 (s, 2H), 3.60 (t, J=6.7 Hz, 2H), 2.38 (t, J=7.3 Hz, 2H), 2.00-1.84 (m, 2H).

### Example 146. Synthesis of 2,5-dioxopyrrolidin-1-yl 4-(2,5-dioxo-2,5-dihydro-1H-pyrrol-1-yl)butanoate (301)

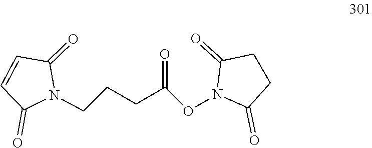 [SMILES: O=C(CCCN1C(=O)C=CC1=O)ON1C(=O)CCC1=O]

4-(2,5-dioxo-2,5-dihydro-1H-pyrrol-1-yl)butanoic acid (400 g, 2.18 mol, 1.0 eq.) was dissolved in CH₂Cl₂ (1.5 L), to which N-hydroxysuccinimide (276 g, 2.40 mmol, 1.1 eq.) and DIC (303 g, 2.40 mol, 1.1 eq.) were added at r.t. and stirred overnight. The reaction was concentrated and purified by column chromatography (1:2 petroleum ether/EtOAc) to give NHS ester as a white solid (382 g, 63% yield). ¹H NMR (500 MHz, CDCl₃) δ 6.74 (s, 2H), 3.67 (t, J=6.8 Hz, 2H), 2.85 (s, 4H), 2.68 (t, J=7.5 Hz, 2H), 2.13-2.03 (m, 2H).

### Example 147. Synthesis of tert-butyl 3-((2-aminoethyl)amino)propanoate (302)

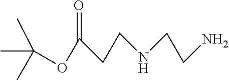

Tert-butyl acrylate (12.81 g, 0.10 mmol) and ethane-1,2-diamine (24.3 g, 0.40 mol) in THF (150 ml) was stirred at 45° C. for 24 h. The mixture was concentrated and purified on Al₂O₃ gel column eluted with Et₃N/MeOH/CH₂Cl₂ (5%:15%:80%) to afford the title compound (17.50 g, 92% yield). ESI MS m/z 189.20 ([M+H]⁺).

### Example 148. Synthesis of 3-((2-aminoethyl)amino)propanoic acid, HCl salt (303)

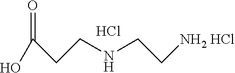

Tert-butyl 3-((2-aminoethyl)amino)propanoate (17.00 g, 90.33 mmol) in 1,4-dioxane (50 ml) was treated with HCl conc. (15 ml). The mixture was stirred at RT for 30 min, concentrated and diluted with pure water (150 ml) and EtOAc/Hexane (40 ml, 1:5). The mixture was separated, and the organic layer was extracted with water (2×10 ml). The aqueous layer was concentrated and dried over vacuum pump to afford the title compound (18.70 g, 100% yield, and 96% pure by LC-MS). ESI MS m/z 133.20 ([M+H]⁺).

### Example 149. 3-((2-(2,5-dioxo-2,5-dihydro-1H-pyrrol-1-yl)ethyl)amino)-propanoic acid (304)

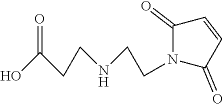 [SMILES: O=C(O)CCNCCN1C(=O)C=CC1=O]

To a solution of 3-((2-aminoethyl)amino)propanoic acid (18.70 g, 90.33 mmol) in THF (150 ml) at 0° C., maleic anhydride (8.85 g, 90.33 mmol) was added. The mixture was stirred at 0-4° C. for 4 h, concentrated to afford (Z)-4-((2-((2-carboxyethyl)amino)ethyl)amino)-4-oxobut-2-enoic acid in quantitative yield confirmed by LC-MS. Then the mixture were added toluene (150 ml) and DMA (50 ml) in, refluxed at 90° C. with Dean-Stark trap. After collected 30 ml solvent in the trap, HMDS (hexamethyldisilazane, 9.0 mL, 43.15 mmol) and ZnCl (16 mL, 1.0 M in diethyl ether) were added. The mixture was heated to 115-125° C., and toluene was collected through a Dean-Stark trap. The reaction mixture was fluxed at 120° C. for 6 h. During this period, 2×40 mL of dry toluene was added to keep the mixture volume around 50 mL. Then the mixture was cooled and 1 mL of 1:10 HCl (conc)/CH₃OH was added in. The mixture was evaporated, and purified on SiO₂ column eluted with water/CH₃CN (1:15), and dried over vacuum pump to afford the title compound 14.75 g (77.0% yield). ESI MS m/z 213.10 ([M+H]).

### Example 150. Synthesis of 2,5,8,11,14,17,20,23-octaoxapentacosan-25-yl 4-methylbenzenesulfonate (305)

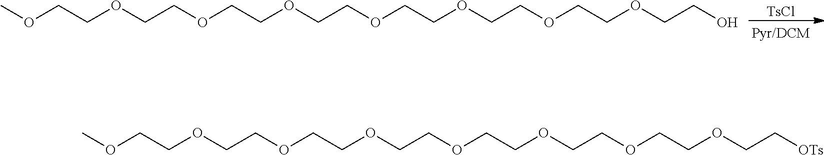 [SMILES: COCCOCCOCCOCCOCCOCCOCCOCCO ; "TsCl" ; COCCOCCOCCOCCOCCOCCOCCOCCOS(=O)(=O)c1ccc(C)cc1]

To a solution of 2,5,8,11,14,17,20,23-Octaoxapentacosan-25-ol (50.0 g, 0.130 mol) in DCM (200 ml) and pyridine (100 ml), TsCl (30.2 g, 0.159 mol) was added. The mixture was stirred overnight, evaporated and purified on SiO₂ column eluted with acetone/DCM (1:1 to 4:1), and dried over vacuum pump to afford the title compound 57.34 g (82.0% yield). ESI MS m/z 539.40 ([M+H]⁺).

### Example 151. Synthesis of S-2,5,8,11,14,17,20,23-octaoxapentacosan-25-yl ethanethioate (306)

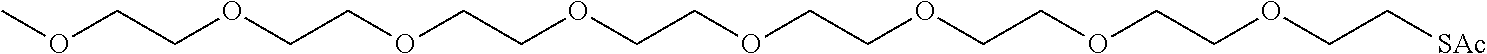

To a solution of 2,5,8,11,14,17,20,23-octaoxapentacosan-25-yl 4-methylbenzenesulfonate (57.30 g, 0.106 mol) in the mixture of THF (300 ml) and DIPEA (50 ml), HSAc (10.0 g, 0.131 mol) was added. The mixture was stirred overnight, evaporated and purified on SiO₂ column eluted with EtOAc/DCM (1:2 to 4:1), and dried over vacuum pump to afford the title compound 40.51 g (86% yield). ESI MS m/z 443.35 ([M+H]⁺).

### Example 152. Synthesis of 2,5,8,11,14,17,20,23-octaoxapentacosane-25-sulfonic acid (307)

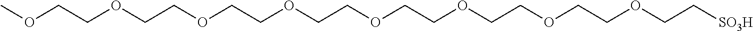 [SMILES: COCCOCCOCCOCCOCCOCCOCCOCCS(=O)(=O)O]

S-2,5,8,11,14,17,20,23-octaoxapentacosan-25-yl ethanethioate (40.40 g, 0.091 mol) in the mixture of acetic acid (200 ml) and 30% H₂O₂ (100 ml) was stirred at 35° C. overnight. The mixture was concentrated, diluted with pure water (200 ml) and toluene (150 ml), separated and the organic layer was extracted with water (2×25 ml). The aqueous solutions were combined, evaporated and dried over vacuum pump to afford the title compound 40.50 g (99% yield, 95% pure by LC-MS). ESI MS m/z 449.30 ([M+H]⁺).

### Example 153. Synthesis of 3,3-N,N-(2″-maleimidoethyl)(2′,5′,8′,11′,14′,17′,20′,23′,26′-nonaoxaoctacosane-28′-sulfin)aminopropanoic acid (308)

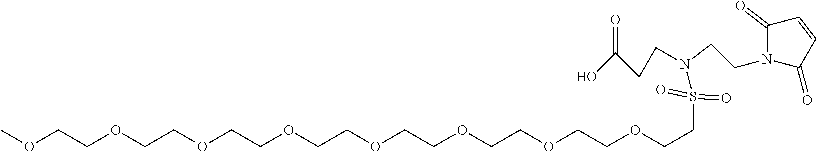 [SMILES: COCCOCCOCCOCCOCCOCCOCCOCCS(=O)(=O)N(CCC(=O)O)CCN1C(=O)C=CC1=O]

To a solution of 2,5,8,11,14,17,20,23-octaoxapentacosane-25-sulfonic acid (20.0 g, 44.62 mmol) in the mixture of THF (100 ml) and DCM (100 ml), (COCl)₂ (25.21 g, 200.19 mmol) and DMF (0.015 ml) was added in sequence. The mixture was stirred at RT for 2 h, concentrated, co-evaporated with DCM/toluene (1:1, 2×50 ml) and then redissolved in THF (50 ml). To the compound of 3-((2-(2,5-dioxo-2,5-dihydro-1H-pyrrol-1-yl)ethyl)amino)-propanoic acid (7.50 g, 35.36 mmol) in THF (100 ml) was added above sulfonyl chloride solution. The mixture was stirred overnight, evaporated in vacuo and purified on SiO₂ column eluted with MeOH/DCM (1:6 to 1:5), and dried over vacuum pump to afford the title compound 14.76 g (65% yield). ESI MS m/z 643.35 ([M+H]⁺).

### Example 154. Synthesis of N— N-succinimido 3,3-N,N-(2″-maleimidoethyl) (2′,5′,8′,11′,14′,17′,20′,23′,26′-nonaoxaoctacosane-28′-sulfin)aminopropanoate (309)

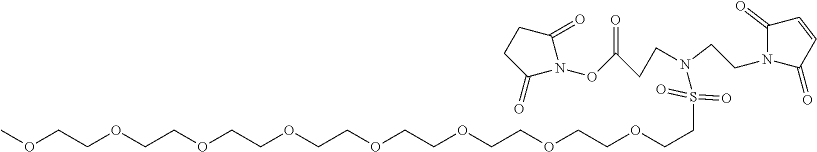 [SMILES: COCCOCCOCCOCCOCCOCCOCCOCCS(=O)(=O)N(CCC(=O)ON1C(=O)CCC1=O)CCN1C(=O)C=CC1=O]

A mixture of 3,3-N,N-(2″-maleimidoethyl)(2′,5′,8′,11′,14′,17′,20′,23′,26′-nonaoxaoctacosane-28′-sulfin)aminopropanoic acid (308) (7.50 g, 11.67 mmol), N-hydroxysuccinimide (1.50 g, 13.04 mmol) and EDC (10.10 g, 52.60 mmol) in THF (100 ml) was stirred overnight, evaporated in vacuo and purified on SiO₂ column eluted with EtOAc/DCM (1:4 to 2:1), and dried over vacuum pump to afford the title compound 6.30 g (73% yield). ESI MS m/z 740.40 ([M+H]⁺).

### Example 155. Synthesis of Compound 310

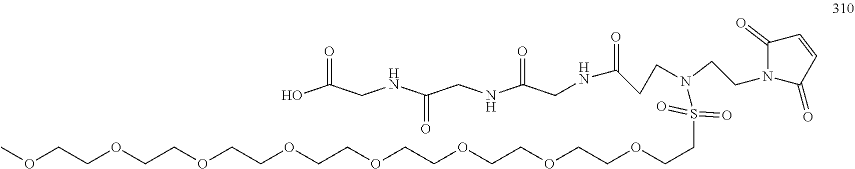 [SMILES: COCCOCCOCCOCCOCCOCCOCCOCCS(=O)(=O)N(CCC(=O)NCC(=O)NCC(=O)NCC(=O)O)CCN1C(=O)C=CC1=O]

A solution of 2-(2-(2-(2-aminoacetamido)acetamido)acetamido)acetic acid (gly-gly-gly) (0.50 g, 2.03 mmol) and compound 309 (1.65 g, 2.22 mmol) in DMF (15 mL) at 0° C., DIPEA (3 mL) was added. The reaction mixture was stirred at 0° C. for 0.5 h, followed by at room temperature for 4 h. Then the reaction mixture was concentrated, and purified by SiO₂ chromatography (mobile phase: acetonitrile/water=95:5 with 0.1% formic acid) to afford the title compound 310 (1.04 g, 63% yield). MS-ESI m/z: [M+H]⁺ calcd for C₃₂H₅₆N₅O₁₇S, 814.33; found, 814.46.

### Example 156. Synthesis of Compound 311

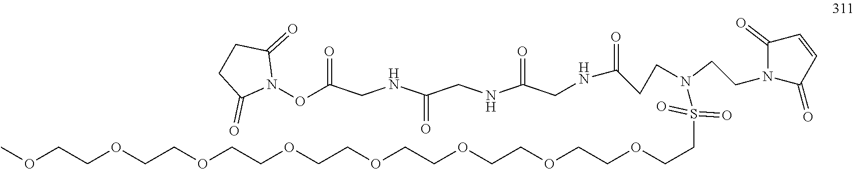 [SMILES: COCCOCCOCCOCCOCCOCCOCCOCCS(=O)(=O)N(CCC(=O)NCC(=O)NCC(=O)NCC(=O)ON1C(=O)CCC1=O)CCN1C(=O)C=CC1=O]

A mixture of compound 310 (0.70 g, 0.86 mmol), N-hydroxysuccinimide (0.20 g, 1.73 mmol) and EDC (1.21 g, 6.36 mmol) in THF (20 ml) was stirred overnight, evaporated in vacuo and purified on SiO₂ column eluted with EtOAc/DCM (1:4 to 2:1), and dried over vacuum pump to afford the title compound 0.540 g (69% yield). MS-ESI m/z: [M+H]⁺ calcd for C₃₆H₅₉N₆O₁₉S, 911.34; found, 911.42.

### Example 157. Synthesis of Compound 312

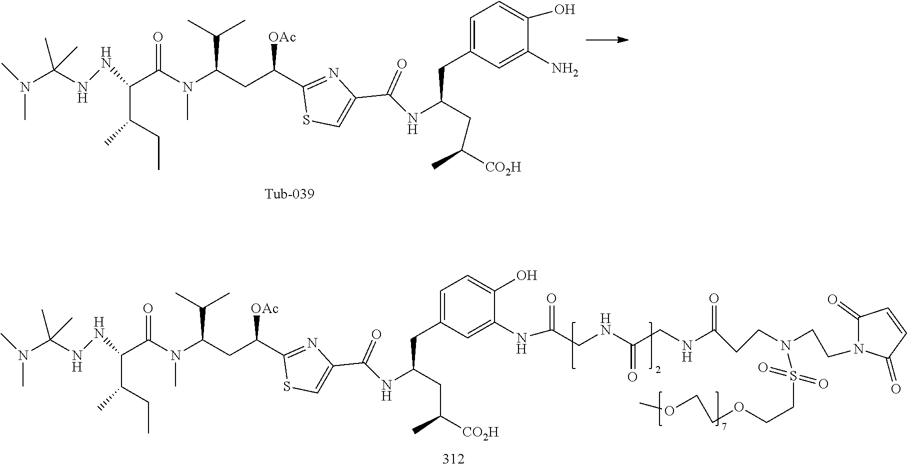

A solution of (2S,4R)-5-(3-amino-4-hydroxyphenyl)-4-(2-((6S,9R,11R)-6-((S)-sec-butyl)-9-isopropyl-2,3,3,8-tetramethyl-4,7,13-trioxo-12-oxa-2,5,8-triazatetradecan-11-yl)thiazole-4-carboxamido)-2-methylpentanoic acid, HCl salt (Tub-039, R. Zhao, et al, PCT/CN2017/120454; R. Zhao, et al, 14th PEGS Boston, Boston, Mass., USA, 3 May 2018) (83 mg, 0.106 mmol) and compound 311 (122 mg, 0.134 mmol) in DMF (8 mL) at 0° C., DIPEA (2 mL) was added. The reaction mixture was stirred at 0° C. for 0.5 h, followed by at room temperature for 4 h. Then the reaction mixture was concentrated, and purified by prep-HPLC (mobile phase: acetonitrile/water=10% to 80% with 0.1% formic acid) to afford compound 312 (95.5 mg, 58% yield). MS-ESI m/z: [M+H]⁺ calcd for C₆₉H₁₁₂N₁₁O₂₄S, 1542.72; found, 1542.76.

### Example 158. Synthesis of Compound 313

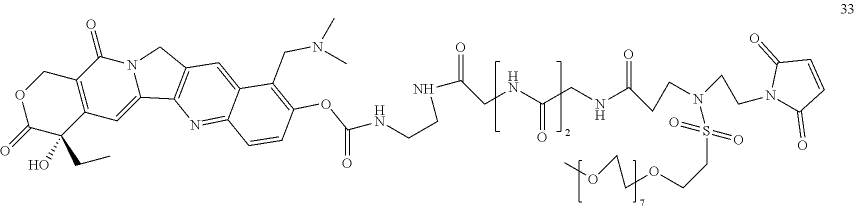

To a solution of compound 243 (40 mg, 0.065 mmol) and compound 311 (71.1 mg, 0.078 mmol) in DMF (5 mL) at 0° C., DIPEA (1 mL) was added. The reaction mixture was stirred at 0° C. for 0.5 h, followed by at room temperature for 4 h. Then the reaction mixture was concentrated and purified by prep-HPLC (mobile phase: acetonitrile/water=10% to 80% with 0.1% formic acid) to afford compound 313 (43.0 mg, 51% yield). MS-ESI m/z: [M+H]⁺ calcd for C₅₈H₈₃N₁₀O₂₂S, 1303.53; found, 1303.58.

### Example 159. Synthesis of (S)-1-benzyl 5-tert-butyl 2-(14-(benzyloxy)-14-oxotetradecanamido)pentanedioate (314)

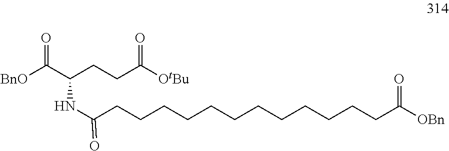 [SMILES: CC(C)(C)OC(=O)CC[C@H](NC(=O)CCCCCCCCCCCCC(=O)OCc1ccccc1)C(=O)OCc1ccccc1]

A solution of (S)-1-benzyl 5-tert-butyl 2-aminopentanedioate, HCl salt (8.70 g, 26.39 mmol), 14-(benzyloxy)-14-oxotetradecanoic acid (9.19 mmol), DIPEA (8.0 ml, 46.0 mmol) and EDC (15.3 g, 80.50 mmol) in CH₂Cl₂ (200 ml) was stirred at room temperature for 6 hour. The mixture was diluted with water (100 ml) and separated. The aqueous phase was extracted with CH₂Cl₂ (100 ml). The organic phases were combined, washed with brine, dried over Na₂SO₄, filtered, concentrated and purified on a silica gel column (dichloromethane/EtOAc=20:1 to 5:1) to give the title compound 314 (13.65 g, 83% yield). MS-ESI m/z: [M+H]⁺ calcd for C₃₇H₅₄NO₇, 624.38; found, 624.38.

### Example 160. Synthesis of (S)-5-(benzyloxy)-4-(14-(benzyloxy)-14-oxotetradecanamido)-5-oxopentanoic acid (315)

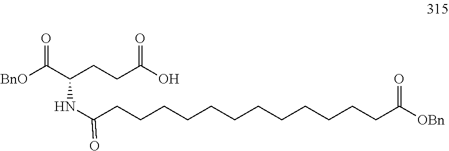 [SMILES: O=C(O)CC[C@H](NC(=O)CCCCCCCCCCCCC(=O)OCc1ccccc1)C(=O)OCc1ccccc1]

Compound 214 (12.50 g, 20.05 mmol) was dissolved in dioxane (30 mL) at 4° C., and treated with hydrochloric acid (10 mL, 36% conc) for 0.5 hours. The reaction mixture was diluted with toluene (20 ml) and DMF (20 ml), evaporated at 15° C. to give the title compound 315 (11.26 g, 99% yield). MS-ESI m/z: [M+H]⁺ calcd for C₃₃H₄₆NO₇, 568.32; found, 568.34.

### Example 161. Synthesis of (S)-35,49-dibenzyl 1-tert-butyl 16,32,37-trioxo-3,6,9,12,19,22,25,28-octaoxa-15,31,36-triazanonatetracontane-1,35,49-tricarboxylate (316)

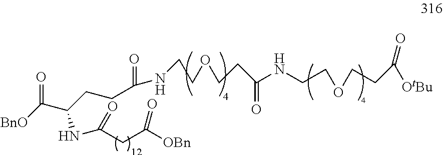

A mixture of compound 315 (10.70 g, 18.86 mmol), tert-butyl 1-amino-15-oxo-3,6,9,12,19,22,25,28-octaoxa-16-azahentriacontan-31-oate HCl salt (11.45 g, 18.93 mmol), EDC (9.51 g, 50.01 mmol) and DIPEA (4.00 ml, 23.00 mol) in CH₂Cl₂ (200 ml) was stirred overnight, diluted with brine (100 ml) and separated. The aqueous phase was extracted with CH₂Cl₂ (100 ml). The organic phases were combined, washed with brine, dried over Na₂SO₄, filtered, concentrated and purified on a silica gel column (dichloromethane/EtOAc=10:1 to 4:1) to give the title compound 316 (18.15 g, 86% yield). MS-ESI m/z: [M+H]⁺ calcd for C₅₉H₉₆N₃O₁₇, 1118.67; found, 1118.80.

### Example 162. Synthesis of(S)-18-((benzyloxy)carbonyl)-3,16,21,37-tetraoxo-1-phenyl-2,25,28,31,34,41,44,47,50-nonaoxa-17,22,38-triazatripentacontan-53-oic acid (317)

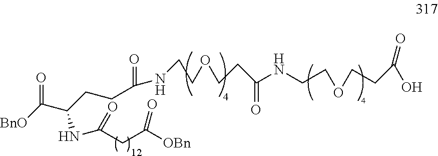

Compound 316 (10.50 g, 9.39 mmol) was dissolved in dioxane (45 mL) at 4° C., and treated with hydrochloric acid (15 mL, 36% conc) for 0.5 hours. The reaction mixture was diluted with toluene (20 ml) and DMF (20 ml), evaporated at 15° C. and purified on a silica gel column (dichloromethane/MeOH=10:1 to 6:1) to give the title compound 317 (8.67 g, 87% yield). MS-ESI m/z: [M+H]⁺ calcd for C₅₅H₈₈N₃O₁₇, 1062.60; found, 1062.68.

### Example 163. Synthesis of (18S,59S)-18-((benzyloxy)carbonyl)-59-((tert-butoxycarbonyl)amino)-3,16,21,37,53-pentaoxo-1-phenyl-2,25,28,31,34,41,44,47,50-nonaoxa-17,22,38,54-tetraazahexacontan-60-oic acid (318)

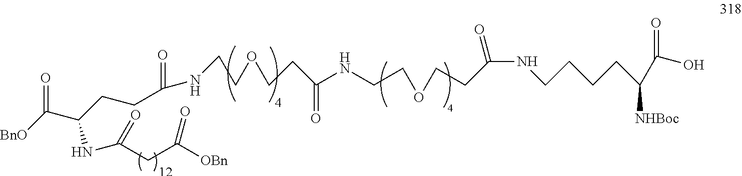

A solution of compound 316 (8.50 g, 8.01 mmol), N-hydroxysuccinimide (3.20 g, 27.82 mmol), EDC (10.28 g, 54.10 mmol) and DIPEA (6.00 ml, 34.51 mmol) in THF (150 ml) was stirred for 6 h and evaporated in vacuo to get a crude N-succinimidyl ester of (S)-18-((benzyloxy)carbonyl)-3,16,21,37-tetraoxo-1-phenyl-2,25,28,31,34,41,44,47,50-nonaoxa-17,22,38-triazatripentacontan-53-oic acid for use in next step without purification.

To a solution of (S)-6-amino-2-((tert-butoxycarbonyl)amino)hexanoic acid, HCl salt (2.75 g, 9.73 mmol) in DMF (100 mL) and 1.0 M Na₂PO₄ (pH 7.5, 55 mL), the above prepared N-succinimidyl ester was added in four portion in 1 h. The reaction mixture was stirred at room temperature for another 3 hour. After concentration, the residue was purified on a silica gel column (dichloromethane/MeOH=10:1 to 4:1) to give the title compound 318 (8.16 g, 79% yield). MS-ESI m/z: [M+H]⁺ calcd for C₆₆H₁₀₈N₅O₂₀, 1289.75; found, 1289.90.

### Example 164. Synthesis of (18S,59S)-59-amino-18-((benzyloxy)carbonyl)-3,16,21,37,53-pentaoxo-1-phenyl-2,25,28,31,34,41,44,47,50-nonaoxa-17,22,38,54-tetraazahexacontan-60-oic acid, HCl salt (319)

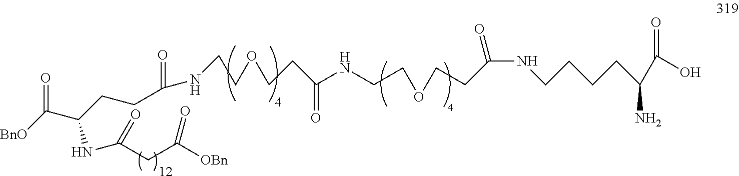

Compound 318 (8.10 g, 6.28 mmol) was dissolved in dioxane (40 mL) at 4° C., and treated with hydrochloric acid (15 mL, 36% conc) for 0.5 hours. The reaction mixture was diluted with toluene (20 ml) and DMF (20 ml), evaporated at 15° C. to give the crude title compound 319 (7.71 g, 100% yield) for next step without further purification. MS-ESI m/z: [M+H]⁺ calcd for C₆₁H₈₈N₃O₁₇, 1190.70; found, 1190.78.

### Example 165. Synthesis of (S)-2-(4-(2,5-dioxo-2,5-dihydro-1H-pyrrol-1-yl)butanamido)propanoic acid (320)

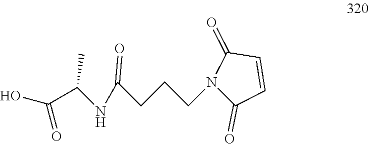 [SMILES: C[C@H](NC(=O)CCCN1C(=O)C=CC1=O)C(=O)O]

To a solution of compound 301 (7.10 g, 25.35 mmol) and alanine (3.01 g, 33.80 mmol) in DMF (50 mL) at 0° C., DIPEA (10 mL) was added. The reaction mixture was stirred at 0° C. for 0.5 h, followed by at room temperature for 1 h. Then the reaction mixture was concentrated and purified on SiO₂ column (mobile phase: DCM/MeOH=10:1 with 0.1% formic acid) to afford compound 320 (5.21 g, 81% yield). MS-ESI m/z: [M+H]⁺ calcd for C₁₁H₁₄N₂O₅, 255.09; found, 255.15.

### Example 166. Synthesis of (S)-2,5-dioxopyrrolidin-1-yl 2-(4-(2,5-dioxo-2,5-dihydro-1H-pyrrol-1-yl)butanamido)propanoate (126)

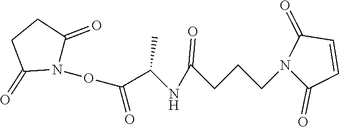 [SMILES: C[C@H](NC(=O)CCCN1C(=O)C=CC1=O)C(=O)ON1C(=O)CCC1=O]

A solution of compound 320 (5.15 g, 20.26 mmol), N-hydroxysuccinimide (2.80 g, 24.34 mmol), EDC (10.28 g, 54.10 mmol) and DIPEA (5.50 ml, 31.63 mmol) in DCM (70 ml) was stirred for 6 h, evaporated in vacuo and purified on SiO₂ column (mobile phase: DCM/EtOAc=10:1) to afford compound 126 (5.83 g, 82% yield). MS-ESI m/z: [M+H]⁺ calcd for C₁₅H₁₇N₃O₇, 351.11; found, 351.20.

### Example 167. Synthesis of (18S,59S)-18-((benzyloxy)carbonyl)-59-((S)-2-(4-(2,5-dioxo-2,5-dihydro-1H-pyrrol-1-yl)butanamido)propanamido)-3,16,21,37,53-pentaoxo-1-phenyl-2,25,28,31,34,41,44,47,50-nonaoxa-17,22,38,54-tetraazahexacontan-60-oic acid (127)

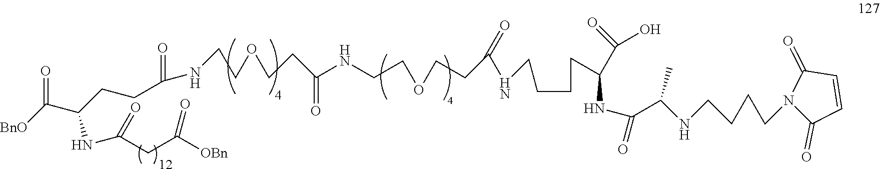

To a solution of compound 319 (7.61 g, 6.39 mmol) and compound 126 (2.90 g, 8.280 mmol) in DMF (40 mL) at 0° C., DIPEA (7 mL) was added. The reaction mixture was stirred at 0° C. for 0.5 h, followed by at room temperature for 1 h. Then the reaction mixture was concentrated and purified on SiO₂ column (mobile phase: DCM/MeOH=10:1 with 0.1% formic acid) to afford compound 127 (7.10 g, 78% yield). MS-ESI m/z: [M+H]⁺ calcd for C₇₂H₁₂N₇O₂₂, 1426.7782; found, 1426.7820.

### Example 168. Synthesis of (18S,59S)-18-((benzyloxy)carbonyl)-59-((S)-2-(4-(2,5-dioxo-2,5-dihydro-1H-pyrrol-1-yl)butanamido)propanamido)-3,16,21,37,53,60,63,66,69-nonaoxo-1-phenyl-2,25,28,31,34,41,44,47,50-nonaoxa-17,22,38,54,61,64,67,70-octaazadoheptacontan-72-oic acid (129)

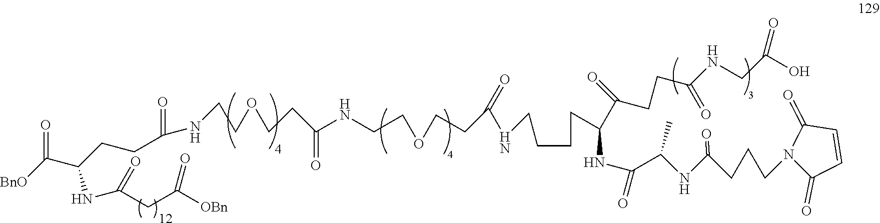

A solution of compound 127 (7.05 g, 4.94 mmol), N-hydroxysuccinimide (0.92 g, 8.00 mmol), EDC (3.01 g, 15.84 mmol) and DIPEA (1.00 ml, 5.75 mmol) in THF (50 ml) was stirred for 6 h and evaporated in vacuo to get a crude compound 128 (N-succinimidyl ester) for use in next step without purification.

To a solution of 2-(2-(2-aminoacetamido)acetamido)acetic acid (gly-gly-gly) HCl salt (1.67 g, 7.40 mmol) in DMF (40 mL) and 1.0 M Na₂PO₄ (pH 7.5, 15 mL), the above compound 128 was added in four portion in 1 h. The reaction mixture was stirred at room temperature for another 3 hour. After concentration, the residue was purified on a silica gel column (dichloromethane/MeOH=10:1 to 7:1) to give the title compound 129 (8.16 g, 79% yield). MS-ESI m/z: [M+H]⁺ calcd for C₇₈H₁₂₁N₁₀O₂₅, 1597.8426; found, 1597.8495.

### Example 169. Synthesis of Compound 130

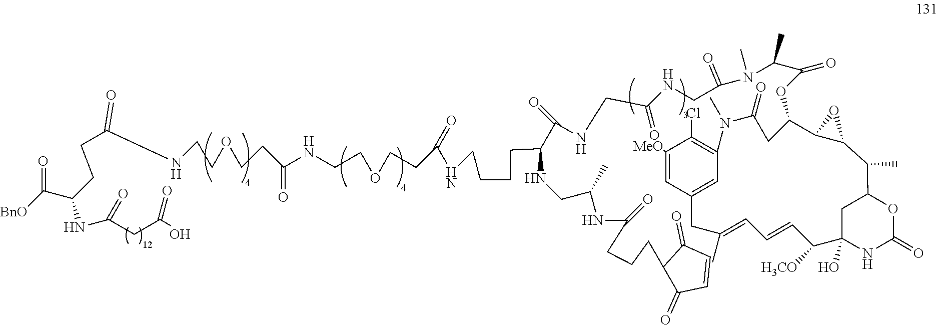

A solution of compound 129 (251 mg, 0.157 mmol), May-NMA (100 mg, ˜0.154 mmol), and DIPEA (0.10 ml, 0.575 mmol) in DMA (10 ml), BroP (bromo tris(dimethylamino) phosphonium hexafluorophosphate) (451 mg, 1.162 mmol) was added. The reaction mixture was stirred at room temperature for 6 h, evaporated in vacuo and purified on a silica gel column (dichloromethane/MeOH=10:1 to 7:1) to afford the compound 130 (212.6 mg, 62% yield). MS-ESI m/z: [M+H]⁺ calcd for C₁₁₀H₁₆₃ClN₁₃O₃₃, 2229.1088; found, 2229.1175.

### Example 170. Synthesis of Compound 131

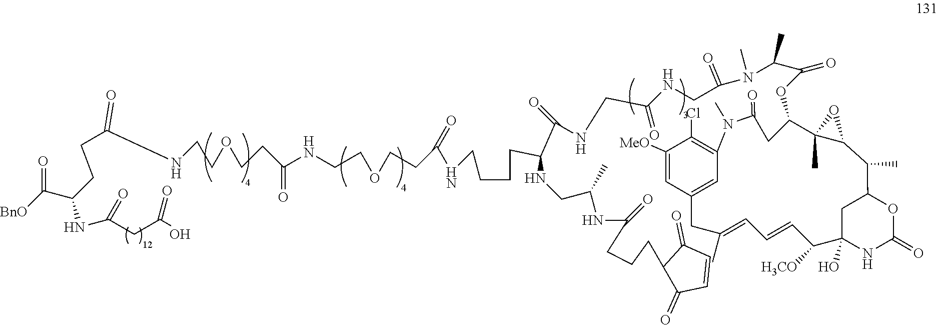

Compound 130 (105 mg, 0.0471 mmol) in DCM (2 mL) was treated with TFA (4 mL) for 1 hours. The reaction mixture was diluted with toluene (5 ml) and DMF (5 ml), evaporated, and purified by prep-HPLC (mobile phase: acetonitrile/water=10% to 80% with 0.10% formic acid) to afford compound 131 (69.0 mg, 72% yield). MS-ESI m/z: [M+H]⁺ calcd for C₉₆H₁₅₁ClN₁₃O₃₃, 2049.0149; found, 2049.0285.

### Example 171. Synthesis of Compound 134

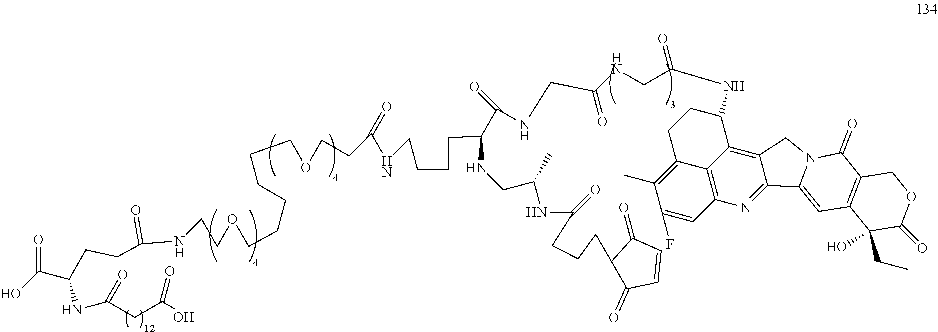

A solution of compound 129 (299.5 mg, 0.187 mmol), exatecan HCl salt (80.5 mg, 0.170 mmol), and DIPEA (0.050 ml, 0.287 mmol) in DMA (10 ml), EDC (200 mg, 1.052 mmol) was added. The reaction mixture was stirred at room temperature for 6 h, evaporated in vacuo, re-dissolved in EtOAc/DCM (1 ml: 4 ml) and passed through a short silica gel column with elution of EtOAc/DCM (1:2), evaporated in vacuo to afford a crude compound for next step. MS-ESI m/z: 2015.01.

The crude above compound in DCM (2 mL) was treated with TFA (4 mL) for 1 hours. The reaction mixture was diluted with toluene (5 ml) and DMF (5 ml), evaporated, and purified by prep-HPLC (mobile phase: 5% to 50% of acetonitrile in water containing 0.1% formic acid) to afford compound 134 (69.0 mg, 72% yield). MS-ESI m/z: [M+H]⁺ calcd for C₈₈H₁₂₈FN₁₃O₂₈, 1834.8980; found, 1834.9010.

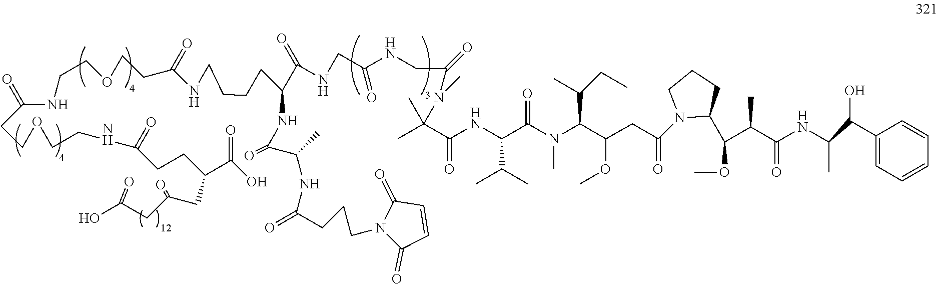

### Example 172. Synthesis of Compound 321

A solution of compound 129 (150.1 mg, 0.0935 mmol), MMAE HCl salt (50.1 mg, 0.0682 mmol), and DIPEA (0.030 ml, 0.172 mmol) in DMA (5 ml), BroP (bromo tris(dimethylamino) phosphonium hexafluorophosphate) (180.1 mg, 0.463 mmol) was added. The reaction mixture was stirred at room temperature for 6 h, evaporated in vacuo, re-dissolved in EtOAc/DCM (1 ml: 4 ml) and passed through a short silica gel column with elution of EtOAc/DCM (1:2), evaporated in vacuo to afford a crude compound for next step. MS-ESI m/z: 2283.3290.

The crude above compound in DCM (1 mL) was treated with TFA (3 mL) for 1 hours. The reaction mixture was diluted with toluene (3 ml) and DMF (2 ml), evaporated, and purified by prep-HPLC (mobile phase: 5% to 50% of acetonitrile in water containing 0.1% formic acid) to afford compound 321 (84.5 mg, 59% yield). MS-ESI m/z: [M+H]⁺ calcd for C₁₀₂H₁₇₂N₁₅O₃₁, 2103.2343; found, 2103.2425.

### Example 173. Synthesis of Compound 322

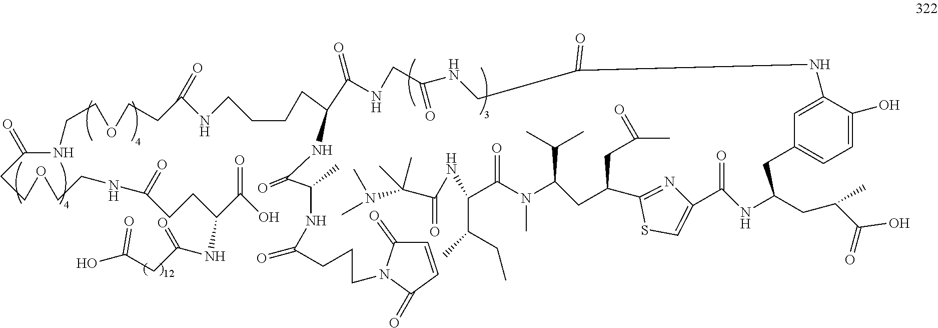

A solution of compound 129 (150.3 mg, 0.0935 mmol), Tub-039 HCl salt (60.2 mg, 0.0769 mmol), and DIPEA (0.030 ml, 0.172 mmol) in DMA (5 ml), EDC (100 mg, 0.526 mmol) was added. The reaction mixture was stirred at room temperature for 6 h, evaporated in vacuo, re-dissolved in MeOH/DCM (0.5 ml: 3 ml) and passed through a short silica gel column with elution of MeOH/DCM (1:3), evaporated in vacuo to afford a crude compound for next step. MS-ESI m/z: 2326.25.

The crude above compound in DCM (1 mL) was treated with TFA (3 mL) for 1 hours. The reaction mixture was diluted with toluene (3 ml) and DMF (3 ml), evaporated, and purified by prep-HPLC (mobile phase: 2% to 50% of acetonitrile in water containing 0.1% formic acid) to afford compound 322 (69.0 mg, 72% yield). MS-ESI m/z: [M+H]⁺ calcd for C₈₈H₁₂₈FN₁₃O₂₈, 2146.1497; found, 2146.1588.

### Example 174. General Method of Preparation of Conjugate 49 (C-30), 50 (C-40), 51 (C-48), 174 (C-173), C-238, C-247, C-255, C-260, C-265, C-271, C-273, C-276, C-279, C-280, C-281, C-312, C-313, 132 (C-131), 135 (C-134), C-321, and C-322

To a solution containing 2.0 mL of 10 mg/ml Herceptin in pH 6.0˜8.0 PBS buffer, 0.70˜2.0 mL of 100 mM NaH₂PO₄, pH 6.5˜8.5 buffers and TCEP (14˜45 μL, 20 mM in water), the compound 30, 40, 48, 173, 238, 247, 255, 260, 265, 271, 273, 276, 279, 281, 312, 313, 131, 134, 321, and 322 (14-60 μL, 20 mM in DMA) were added independently, followed by addition of 4-(azidomethyl)benzoic acid (14-70 μL, 20 mM in pH 7.5, PBS buffer). The mixture was incubated at RT for 4˜18 h, then DHAA (125˜160 μL, 50 mM) was added in. After continuous incubation at RT overnight, the mixture was purified on G-25 column eluted with 100 mM NaH₂PO₄, 50 mM NaCl pH 6.0˜7.5 buffer to afford 11.2˜18.5 mg of the conjugate compounds 49 (C-30), 50 (C-40), 51 (C-48), 174 (C-173), C-238, C-247, C-255, C-260, C-265, C-271, C-273, C-276, C-279, C-280, C-281, C-312, C-313, 132 (C-131), 135 (C-134), C-321, and C-322 (85%˜94% yield) accordingly in 13.4˜15.8 ml of the NaH₂PO₄, buffer. The drug/antibody ratio (DAR) was 3.5˜8.2 for conjugate, wherein DAR was determined via UPLC-QTOF mass spectrum. It was 95˜99% monomer analyzed by SEC HPLC (Tosoh Bioscience, Tskgel G3000SW, 7.8 mm ID×30 cm, 0.5 ml/min, 100 min). The structures of these conjugates: C-238, C-247, C-255, C-260, C-265, C-271, C-273, C-276, C-279, C-280, C-281, C-312, C-313, C-321, and C-322, which are not listed in the file of Figures, are illustrated below:

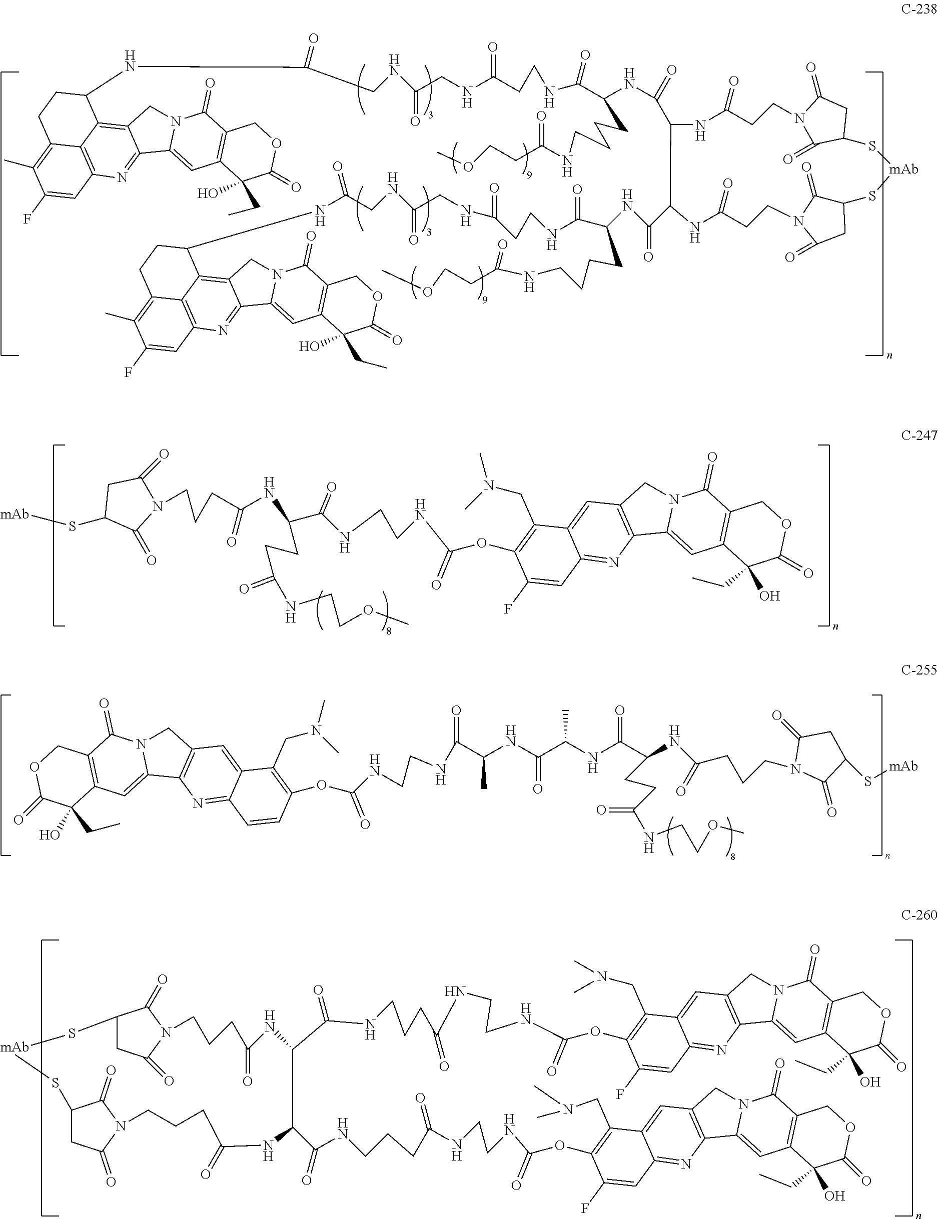

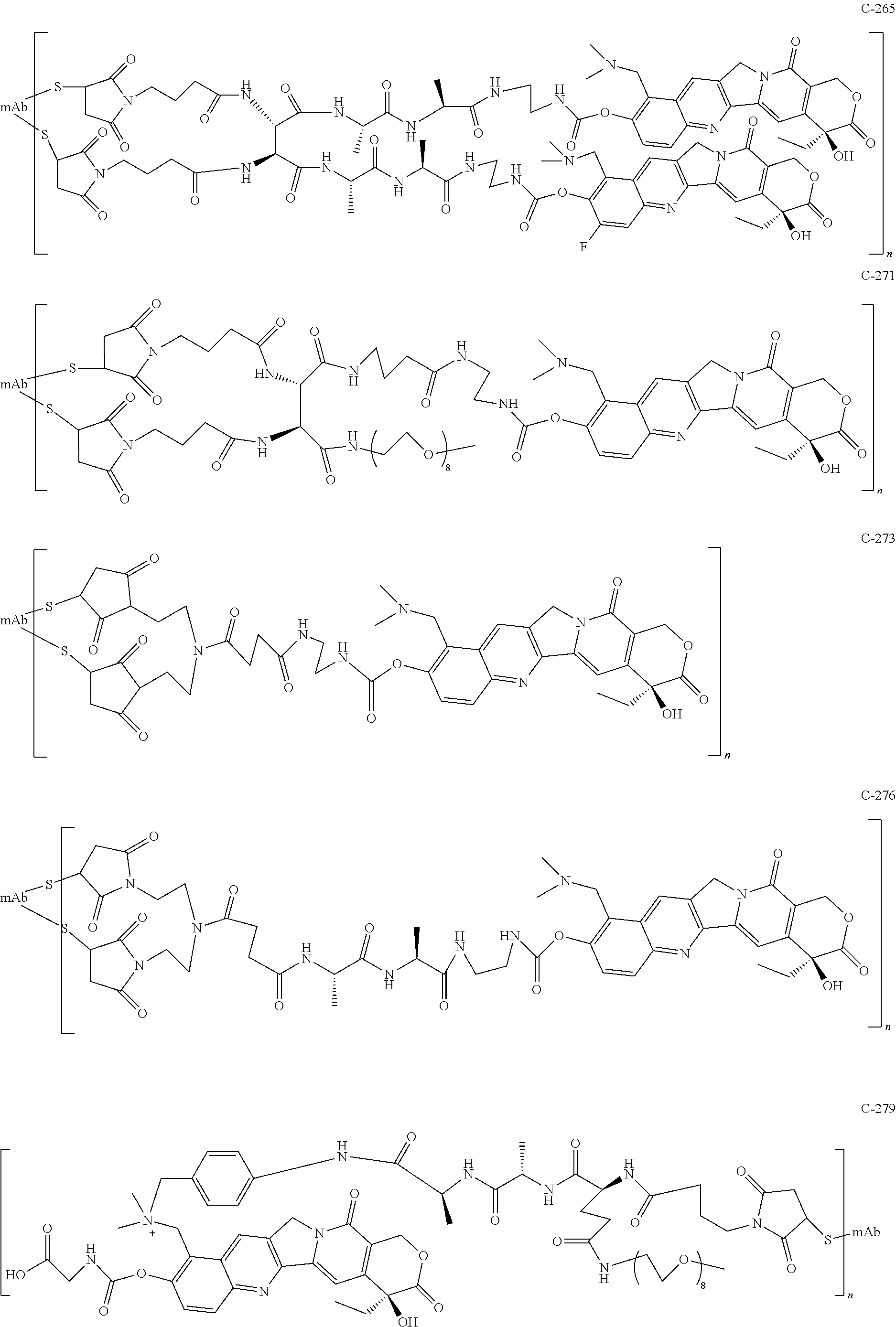

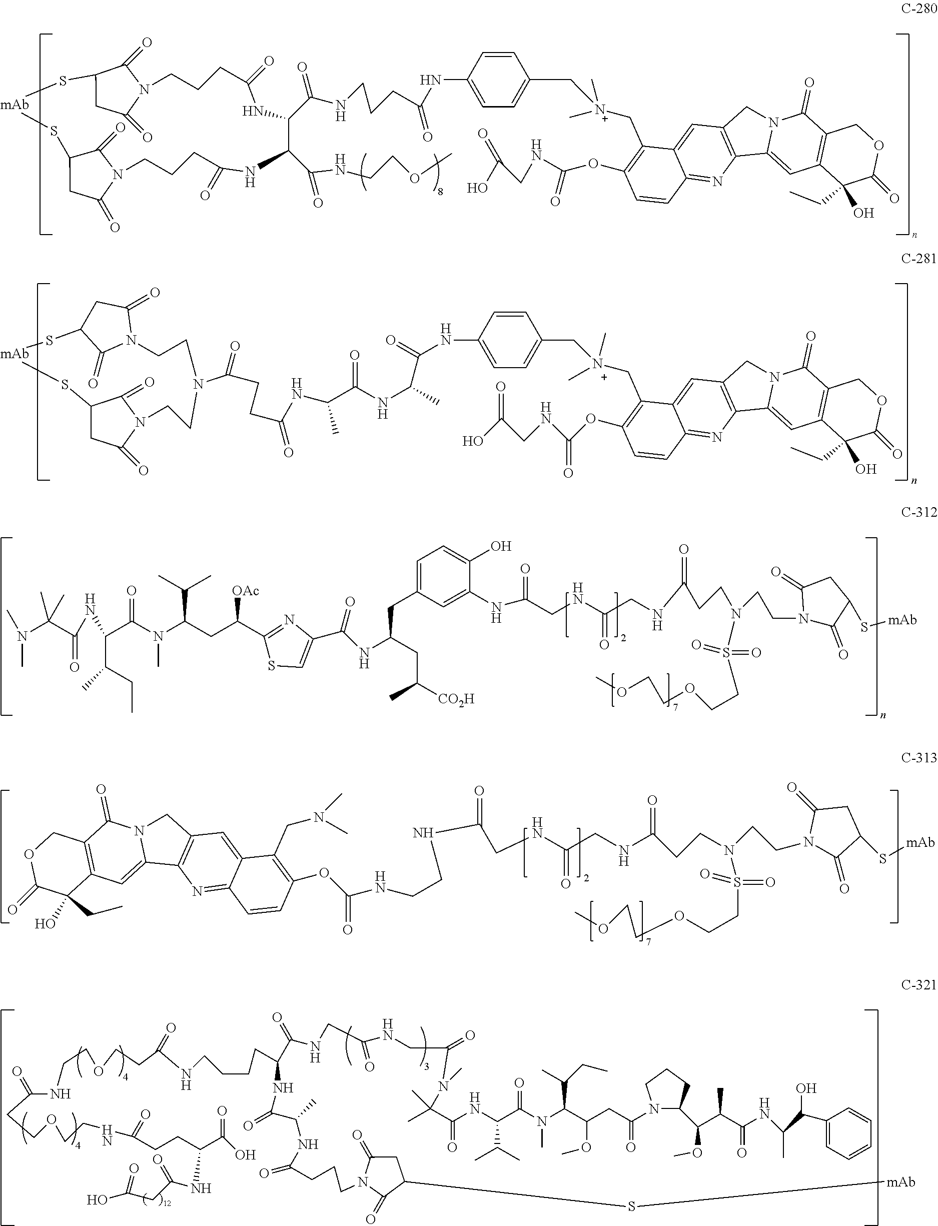

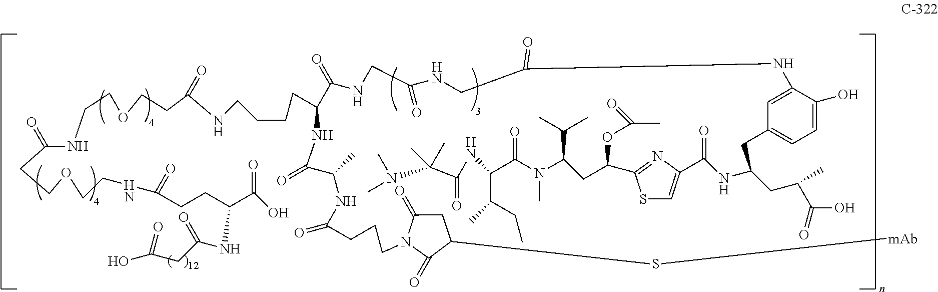

### Example 175. In Vitro Cytotoxicity Evaluation of 49 (C-30), 50 (C-40), 51 (C-48), 174 (C-173), C-238, C-247, C-255, C-260, C-265, C-271, C-273, C-276, C-279, C-280, C-281, C-312, C-313, 132 (C-131), 135 (C-134), C-321, and C-322, in comparison with T-DM1

The cell line used in the cytotoxicity assays was NCI-N87, a human gastric carcinoma cell line; The cells were grown in RPMI-1640 with 10% FBS. To run the assay, the cells (180 μl, 6000 cells) were added to each well in a 96-well plate and incubated for 24 hours at 37° C. with 5% CO₂. Next, the cells were treated with test compounds (20 μl) at various concentrations in appropriate cell culture medium (total volume, 0.2 mL). The control wells contain cells and the medium but lack the test compounds. The plates were incubated for 120 hours at 37° C. with 500 CO₂. MTT (5 mg/ml) was then added to the wells (20 μl) and the plates were incubated for 1.5 hr at 37° C. The medium was carefully removed and DMSO (180 μl) was added afterward. After it was shaken for 15 min, the absorbance was measured at 490 nm and 570 nm with a reference filter of 620 nm. The inhibition % was calculated according to the following equation: inhibition %=[1−(assay-blank)/(control-blank)]×100. The results are listed in Table 1.

TABLE 1
The Structures of the Her2-amatoxin analog conjugates of the patent
application along with their cytotoxicity IC₅₀ results:

| | DAR (drug/mAb | IC50 (nM) against |
|---|---|---|
| Compound | ratio) | NCI-N87 cells |
| C-30 | 3.9 | 0.16 |
| C-40 | 4.0 | 0.27 |
| C-48 | 3.8 | 0.12 |
| C-173 | 7.8 | 6.81 |
| C-238 | 7.6 | 1.58 |
| C-247 | 8.1 | 29.72 |
| C-255 | 8.0 | 21.3 |
| C-260 | 7.6 | 72.81 |
| C-265 | 7.7 | 16.53 |
| C-271 | 4.1 | 185.40 |
| C-273 | 5.4 | 121.72 |
| C-276 | 5.2 | 82.52 |
| C-279 | 7.8 | 7.21 |
| C-280 | 4.8 | 27.35 |
| C-281 | 4.6 | 29.20 |
| C-312 | 4.0 | 0.15 |
| C-313 | 8.1 | 11.10 |
| C-131 | 3.6 | 0.68 |
| C-134 | 7.8 | 1.32 |
| C-321 | 3.9 | 0.95 |
| C-322 | 3.9 | 0.25 |
| T-DM1 | 3.5 | 0.32 |

### Example 176. Antitumor Activity In Vivo (BALB/c Nude Mice Bearing NCI-N87 Xenograft Tumor)

The in vivo efficacy of conjugates 49 (C-30), 51 (C-48), C-173, C-238, C-312, 132 (C-131), 135 (C-134), C-321, and C-322, along with T-DM1 were evaluated in a human gastric carcinoma N-87 cell line tumor xenograft models. Five-week-old female BALB/c Nude mice (66 animals) were inoculated subcutaneously in the area under the right shoulder with N-87 carcinoma cells (5×10⁶ cells/mouse) in 0.1 mL of serum-free medium. The tumors were grown for 8 days to an average size of 140 mm³. The animals were then randomly divided into 10 groups (6 animals per group). The first group of mice served as the control group and was treated with the phosphate-buffered saline (PBS) vehicle. 10 groups were treated with conjugates 49 (C-30), 51 (C-48), C-238, C-312, 132 (C-131), 135 (C-134), C-321, C-322, and T-DM1 respectively at dose of 6 mg/Kg administered intravenously. Three dimensions of the tumor were measured every 3 or 4 days (twice a week) and the tumor volumes were calculated using the formula tumor volume=½(length×width×height). The weight of the animals was also measured at the same time. A mouse was sacrificed when any one of the following criteria was met: (1) loss of body weight of more than 20% from pretreatment weight, (2) tumor volume larger than 1500 mm³, (3) too sick to reach food and water, or (4) skin necrosis. A mouse was considered to be tumor-free if no tumor was palpable.

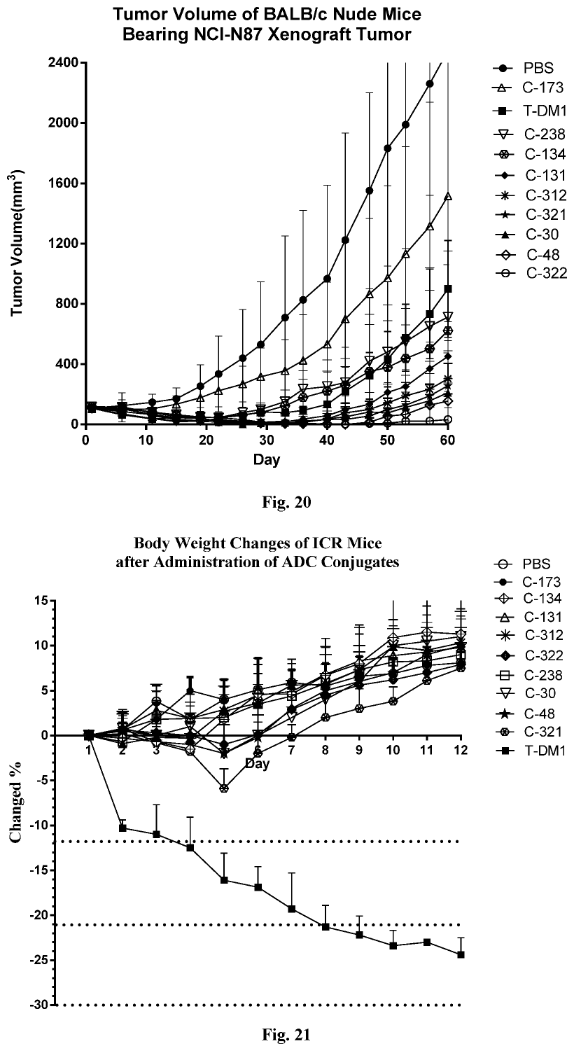

The results were plotted in FIG. 20. All the 10 conjugates did not cause the animal body weight loss at dose of 6.0 mg/Kg. All conjugates demonstrated antitumor activity as comparison with PBS buffer. All conjugates except C-173 had better antitumor activity in vivo than T-DM1. Conjugate C-131, which has the same payload of maytansinoid as T-DM1, delayed tumor growth for 40 days in comparison with 30 days by T-DM1, demonstrating the slow release ability of the side-chain linker.

Here all 6/6 animals at the groups of the tested conjugates had almost no tumor measurable at day 18 till day 32-48. The inhibition of the tumor growth at dose of 6 mg/Kg is:

| Conjugate | Tumor growth delay | |
|---|---|---|
| C-173 | 10 | |
| T-DM1 | 30 | days |
| C-238 | 30 | days |
| C-134 | 28 | days |
| C-131 | 40 | days |
| C-312 | 47 | days |
| C-321 | 48 | days |
| C-30 | 49 | days |
| C-48 | 52 | days |
| C-322 | >54 | days |

### Example 177. Toxicity Study of the Conjugates Having a Side Chain-Linkage in Comparison with T-DM1

Change (typically reduction) in body weight is animal's general response to drug toxicities. 88 female ICR mice, 6-7 weeks old, were separated into 11 groups. Each group included 8 mice and each mouse was given conjugates 49 (C-30), 51 (C-48), C-173, C-238, C-312, 132 (C-131), 135 (C-134), C-321, and C-322, and T-DM1, respectively at dose of 150 mg/Kg per mouse, i.v. bolus. A control group (n=8) was set by I.V. dosing vehicle solution, phosphate buffered saline (PBS). Body weight (BW) of the control mice and all conjugates except C-321 and T-DM1 were not reduced more than 5% in 12-days experiment. The maximum of BW reduction in conjugate C-321 was 5.5% at day 5 followed by a quick recovery. In contrast, BW in T-DM1 continued decreasing with a maximal reduction of 24% from pre-dosing value, and no recovery tendency was seen at the end of the study. The BW change experiments demonstrated greater tolerability for these cytotoxic agent conjugates containing a side-chain linker than that of T-DM1 having a regular mono-linker in these mice.

1-21. (canceled)

22. A side chain-linked conjugate compound of the Formula (I): 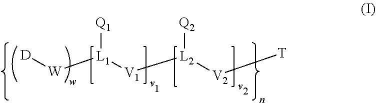
wherein
“ [symbol] ” represents a single bond; n is 1 to 30;
T is a cell-binding agent selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and/or a cell-binding peptide, protein, or small molecule attached on albumin, a polymer, a dendrimer, a liposome, a nanoparticle, a vesicle, or on a (viral) capsid;
L₁ and L₂ are, the same or different, independently O, NH, N, S, P, NNH, NHNH, N(R₃), N(R₁₂), N(R₁₂)N(R_{12′}), CH, CO, C(O)NH, C(O)O, NHC(O)NH, NHC(O)O, polyethyleneoxy unit of formula (OCH₂CH₂)ₚOR₁₂, (OCH₂CH—(CH₃))ₚOR₁₂, NH(CH₂CH₂O)ₚR₁₂, NH(CH₂CH(CH₃)O)ₚR₁₂, N[(CH₂CH₂O)ₚR₁₂]—[(CH₂CH₂O)_{p′}R_{12′}], (OCH₂CH₂)ₚCOOR₁₂, or CH₂CH₂(OCH₂CH₂)ₚCOOR₁₂, wherein p and p′ are independently an integer selected from 0 to about 1000, or a combination of two or more of the above groups; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or (Aa)ᵣ, r=1-12 (one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;
W is a stretcher unit having C₁-C₁₈, and a self-immolative spacer, a peptidyl unit, a hydrazone, a disulfide, a thioether, an ester, or an amide bond;
w is 1 or 2 or 3;
V₁ and V₂ are independently a spacer unit selected from O, NH, S, C₁-C₈ alkyl, C₂-C₈ heteroalkyl, alkenyl, or alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12 (1 to 12 amino acid units), which is composed from a natural or unnatural amino acid, or the same or different sequences of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000;
v₁ and v₂ are independently 0, 1 or 2, but v₁ and v₂ are not 0 at the same time; when v₁ or v₂ is 0, it means one of side chain Q₁ or Q₂ fragment is absent;
Q₁ and Q₂ are independently represented by Formula (I-q1): 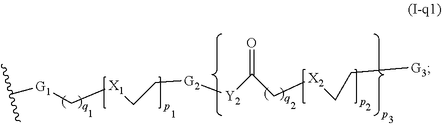
wherein [symbol] is a site linked to L₁ or L₂, G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1}; G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH₂, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}—P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing the same or different sequence of natural or unnatural amino acids; X₁ and X₂ are independently O, CH₂, S, S(O), NHNH, NH, N(R₁₂), ⁺NH(R₁₂), ⁺N(R₁₂)(R_{12′}), C(O), OC(O), OC(O)O, OC(O)NH, NHC(O)NH; Y₂ is O, NH, NR₁₂, CH₂, S, NHNH, or Ar; p₁, p₂ and p₃ are independently 0-100 but are not 0 at the same time; q₁ and q₂ are independently 0-24; R₁₂, R_{12′}, R₁₃ and R_{13′} are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl;
or Q₁ and Q₂ are independently, linear or branched, a C₂-C₁₀₀ polycarboxylacid, a C₂-C₉₀ polyalkylamine, a C₆-C₉₀ oligosaachride or polysaccharide, a C₆-C₁₀₀ zwitterionic betaines or zwitterionic poly(sulfobetaine)) (PSB)s that consist of a quaternary ammonium cation and/or a sulfonate anion, a C₆-C₁₀₀ biodegradable polymer, composed of poly (lactic/glycolic acid) (PLGA), poly(acrylates), chitosan, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), Poly(ethylene glycol) (PEG), poly(propylene glycol) (PPG), poly(lactide-co-glycolide), poly(ethylene glycol)-modified peptide, poly(ethylene glycol)-containing an amino acid or peptide, poly(ethylene glycol)-modified lipid, poly-glycine, poly-N-methyl-glycine, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;
or any one or more of W, Q₁, Q₂, L₁, L₂, V₁, or V₂ can be independently absent but Q₁ and Q₂ are not absent at the same time;
D is a cytotoxic agent that is independently selected from calicheamicins, maytansinoids, camptothecins, taxanes, anthracyclines (daunorubicin/doxorubicin), vinca alkaloids, auristatins, eribulins, (pyrrolo)benzodiazepines (PBDs), CC-106/duocarmycins, tubulysins, amatoxins (such as amanitins), protein kinase inhibitors, MEK inhibitors, KSP inhibitors, nicotinamide phosphoribosyltransferase (NAMPT) inhibitors, immunotoxins, and analogs or prodrugs of these compounds.

23. A side chain-linked conjugate compound of the Formula (II) or (III): 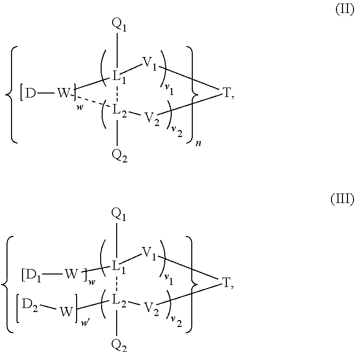
wherein:
“ [symbol] ” represents a single bond; n is 1 to 30;
T is a cell-binding agent selected from the group consisting of an antibody, a single chain antibody, an antibody fragment that binds to a target cell, a monoclonal antibody, a single chain monoclonal antibody, a monoclonal antibody fragment that binds to the target cell, a chimeric antibody, a chimeric antibody fragment that binds to the target cell, a domain antibody, a domain antibody fragment that binds to the target cell, an adnectin that mimics antibody, DARPins, a lymphokine, a hormone, a vitamin, a growth factor, a colony stimulating factor, a nutrient-transport molecule (a transferrin), and/or a cell-binding peptide, protein, or small molecule attached on albumin, a polymer, a dendrimer, a liposome, a nanoparticle, a vesicle, or on a (viral) capsid;
L₁ and L₂ are, the same or different, independently O, NH, N, S, P, NNH, NHNH, N(R₃), N(R₁₂), N(R₁₂)N(R_{12′}), CH, CO, C(O)NH, C(O)O, NHC(O)NH, NHC(O)O, polyethyleneoxy unit of formula (OCH₂CH₂)ₚOR₁₂, (OCH₂CH—(CH₃))ₚOR₁₂, NH(CH₂CH₂O)ₚR₁₂, NH(CH₂CH(CH₃)O)ₚR₁₂, N[(CH₂CH₂O)ₚR₁₂]—[(CH₂CH₂O)_{p′}R_{12′}], (OCH₂CH₂)ₚCOOR₁₂, or CH₂CH₂(OCH₂CH₂)ₚCOOR₁₂, wherein p and p′ are independently an integer selected from 0 to about 1000, or a combination of two or more of the above groups; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or (Aa)ᵣ, r=1-12 (one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;
W is a stretcher unit having C₁-C₁₈, and a self-immolative spacer, a peptidyl unit, a hydrazone, a disulfide, a thioether, an ester, or an amide bond;
V₁ and V₂ are independently a spacer unit selected from O, NH, S, C₁-C₈ alkyl, C₂-C₈ heteroalkyl, alkenyl, or alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12 (1 to 12 amino acid units), which is composed from a natural or unnatural amino acid, or the same or different sequences of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000;
v₁ and v₂ are independently 0, 1 or 2, but v₁ and v₂ are not 0 at the same time; when v₁ or v₂ is 0, it means one of side chain Q₁ or Q₂ fragment is absent;
Q₁ and Q₂ are independently represented by Formula (I-q1): 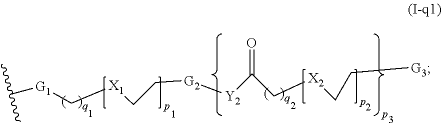
wherein [symbol] is a site linked to L₁ or L₂; G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1}; G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH₂, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}—P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing the same or different sequence of natural or unnatural amino acids; X₁ and X₂ are independently O, CH₂, S, S(O), NHNH, NH, N(R₁₂), ⁺NH(R₁₂), ⁺N(R₁₂)(R_{12′}), C(O), OC(O), OC(O)O, OC(O)NH, NHC(O)NH; Y₂ is O, NH, NR₁₂, CH₂, S, NHNH, or Ar; p₁, p₂ and p₃ are independently 0-100 but are not 0 at the same time; q₁ and q₂ are independently 0-24; R₁₂, R_{12′}, R₁₃ and R_{13′} are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl;
or Q₁ and Q₂ are independently, linear or branched, a C₂-C₁₀₀ polycarboxylacid, a C₂-C₉₀ polyalkylamine, a C₆-C₉₀ oligosaachride or polysaccharide, a C₆-C₁₀₀ zwitterionic betaines or zwitterionic poly(sulfobetaine)) (PSB)s that consist of a quaternary ammonium cation and/or a sulfonate anion, a C₆-C₁₀₀ biodegradable polymer, composed of poly (lactic/glycolic acid) (PLGA), poly(acrylates), chitosan, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), Poly(ethylene glycol) (PEG), poly(propylene glycol) (PPG), poly(lactide-co-glycolide), poly(ethylene glycol)-modified peptide, poly(ethylene glycol)-containing an amino acid or peptide, poly(ethylene glycol)-modified lipid, poly-glycine, poly-N-methyl-glycine, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;
or any one or more of W, Q₁, Q₂, L₁, L₂, V₁, or V₂ can be independently absent but Q₁ and Q₂ are not absent at the same time;
D is a cytotoxic agent that is independently selected from calicheamicins, maytansinoids, camptothecins, taxanes, anthracyclines (daunorubicin/doxorubicin), vinca alkaloids, auristatins, eribulins, (pyrrolo)benzodiazepines (PBDs), CC-106/duocarmycins, tubulysins, amatoxins (such as amanitins), protein kinase inhibitors, MEK inhibitors, KSP inhibitors, nicotinamide phosphoribosyltransferase (NAMPT) inhibitors, immunotoxins, and analogs or prodrugs of these compounds;
w and w′ are independently 1, 2 or 3; and [symbol] is a single bond, double bond or absent, D₁ and D₂ are the same or different, and are defined the same as D.

24. A side chain-linkage compound of Formula (IV), which is capable of reacting with a cell-binding molecule T to form a conjugate: 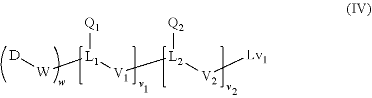
wherein:
“ [symbol] ” represents a single bond; n is 1 to 30;
L₁ and L₂ are, the same or different, independently O, NH, N, S, P, NNH, NHNH, N(R₃), N(R₁₂), N(R₁₂)N(R_{12′}), CH, CO, C(O)NH, C(O)O, NHC(O)NH, NHC(O)O, polyethyleneoxy unit of formula (OCH₂CH₂)ₚOR₁₂, (OCH₂CH—(CH₃))ₚOR₁₂, NH(CH₂CH₂O)ₚR₁₂, NH(CH₂CH(CH₃)O)ₚR₁₂, N[(CH₂CH₂O)ₚR₁₂]—[(CH₂CH₂O)_{p′}R_{12′}], (OCH₂CH₂)ₚCOOR₁₂, or CH₂CH₂(OCH₂CH₂)ₚCOOR₁₂, wherein p and p′ are independently an integer selected from 0 to about 1000, or a combination of two or more of the above groups; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or (Aa)ᵣ, r=1-12 (one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;
W is a stretcher unit having C₁-C₁₈, and a self-immolative spacer, a peptidyl unit, a hydrazone, a disulfide, a thioether, an ester, or an amide bond;
w is 1 or 2 or 3;
V₁ and V₂ are independently a spacer unit selected from O, NH, S, C₁-C₈ alkyl, C₂-C₈ heteroalkyl, alkenyl, or alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12 (1 to 12 amino acid units), which is composed from a natural or unnatural amino acid, or the same or different sequences of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000;
v₁ and v₂ are independently 0, 1 or 2, but v₁ and v₂ are not 0 at the same time; when v₁ or v₂ is 0, it means one of side chain Q₁ or Q₂ fragment is absent;
Q₁ and Q₂ are independently represented by Formula (I-q1): 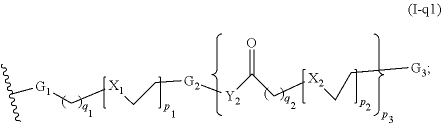
wherein “ [symbol] ” is a site linked to L₁ or L₂; G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1}; G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH₂, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}—P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing the same or different sequence of natural or unnatural amino acids; X₁ and X₂ are independently O, CH₂, S, S(O), NHNH, NH, N(R₁₂), ⁺NH(R₁₂), ⁺N(R₁₂)(R_{12′}), C(O), OC(O), OC(O)O, OC(O)NH, NHC(O)NH; Y₂ is O, NH, NR₁₂, CH₂, S, NHNH, or Ar; p₁, p₂ and p₃ are independently 0-100 but are not 0 at the same time; q₁ and q₂ are independently 0-24; R₁₂, R_{12′}, R₁₃ and R_{13′} are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl;
or Q₁ and Q₂ are independently, linear or branched, a C₂-C₁₀₀ polycarboxylacid, a C₂-C₉₀ polyalkylamine, a C₆-C₉₀ oligosaachride or polysaccharide, a C₆-C₁₀₀ zwitterionic betaines or zwitterionic poly(sulfobetaine)) (PSB)s that consist of a quaternary ammonium cation and/or a sulfonate anion, a C₆-C₁₀₀ biodegradable polymer, composed of poly (lactic/glycolic acid) (PLGA), poly(acrylates), chitosan, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), Poly(ethylene glycol) (PEG), poly(propylene glycol) (PPG), poly(lactide-co-glycolide), poly(ethylene glycol)-modified peptide, poly(ethylene glycol)-containing an amino acid or peptide, poly(ethylene glycol)-modified lipid, poly-glycine, poly-N-methyl-glycine, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;
or any one or more of W, Q₁, Q₂, L₁, L₂, V₁, or V₂ can be independently absent but Q₁ and Q₂ are not absent at the same time;
D is a cytotoxic agent that is independently selected from calicheamicins, maytansinoids, camptothecins, taxanes, anthracyclines (daunorubicin/doxorubicin), vinca alkaloids, auristatins, eribulins, (pyrrolo)benzodiazepines (PBDs), CC-106/duocarmycins, tubulysins, amatoxins (such as amanitins), protein kinase inhibitors, MEK inhibitors, KSP inhibitors, nicotinamide phosphoribosyltransferase (NAMPT) inhibitors, immunotoxins, and analogs or prodrugs of these compounds;
Lv₁ is a reacting group that can be reacted with a thiol, amine, carboxylic acid, selenol, phenol or hydroxyl group on a cell-binding molecule; Lv₁ is OH; F; Cl; Br; I; nitrophenol; N-hydroxysuccinimide (NHS); phenol; dinitrophenol; pentafluorophenol; tetrafluorophenol; difluorophenol; mono-fluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydride formed by acid themselves, or formed with another C₁-C₈ acid anhydride; or an intermediate molecule generated with a condensation reagent for peptide coupling reactions, or for Mitsunobu reactions; the condensation reagent is selected from the group consisting of EDC (N-(3-Dimethylaminopropyl)-N′-ethylcarbodiimide), DCC (Dicyclohexyl-carbodiimide), N,N′-Diisopropylcarbodiimide (DIC), N-Cyclohexyl-N′-(2-morpholino-ethyl)carbodiimide metho-p-toluenesulfonate (CMC, or CME-CDI), 1,1′-Carbonyldiimidazole (CDI), TBTU (0-(Benzotriazol-1-yl)-N,N,N′,N′-tetramethyluronium tetrafluoroborate), N,N,N′,N′-Tetramethyl-O-(1H-benzo-triazol-1-yl)-uronium hexafluoro-phosphate (HBTU), (Benzotriazol-1-yloxy)tris(dimethylamino)-phosphonium hexafluorophosphate (BOP), (Benzotriazol-1-yloxy)tripyrrolidinophosphonium hexafluorophosphate (PyBOP), Diethyl cyanophosphonate (DEPC), Chloro-N,N,N′,N′-tetra-methylformamidiniumhexafluorophosphate, 1-[Bis(dimethyl-amino)methylene]-1H-1,2,3-triazolo[4,5-b]pyridinium 3-oxid hexafluorophosphate (HATU), 1-[(Dimethylamino)-(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetra-methylene)-formamidinium hexafluorophosphate (BTFFH), N,N,N′,N′-Tetramethyl-S-(1-oxido-2-pyridyl)-thiuronium hexafluorophosphate, O-(2-Oxo-1(2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate (HOTU), (1-Cyano-2-ethoxy-2-oxoethylidenaminooxy) dimethylamino-morpholino-carbenium hexafluorophosphate (COMU), O-(Benzotriazol-1-yl)-N,N,N′,N′-bis(tetramethylene)-uronium hexafluorophosphate (HBPyU), N-Benzyl-N′-cyclohexyl-carbodiimide (with, or without polymer-bound), Dipyrrolidino(N-succinimidyl-oxy)carbenium hexafluoro-phosphate (HSPyU), Chlorodipyrrolidinocarbenium hexafluorophosphate (PyClU), 2-Chloro-1,3-dimethylimidazolidinium tetrafluoroborate (CIB), (Benzotriazol-1-yloxy)dipiperidino-carbenium hexafluorophosphate (HBPipU), O-(6-Chlorobenzotriazol-1-yl)-N,N,N′,N′-tetramethyluronium tetrafluoroborate (TCTU), Bromotris(dimethylamino)-phosphonium hexafluorophosphate (BroP), Propylphosphonic anhydride (PPACA, T3P©), 2-Morpholinoethyl isocyanide (MEI), N,N,N′,N′-Tetramethyl-O—(N-succinimidyl)uronium hexafluorophosphate (HSTU), 2-Bromo-1-ethyl-pyridinium tetrafluoroborate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholiniumchloride (MMTM, DMTMM), N,N,N′,N′-Tetramethyl-O—(N-succinimidyl)uronium tetrafluoroborate (TSTU), O-(3,4-Dihydro-4-oxo-1,2,3-benzotriazin-3-yl)-N,N,N′,N′-tetramethyluronium tetrafluoro-borate (TDBTU), 1,1′-(Azodicarbonyl)-dipiperidine (ADD), Di-(4-chlorobenzyl)azodi-carboxylate (DCAD), Di-tert-butyl azodicarboxylate (DBAD), Diisopropyl azodicarboxylate (DIAD), and Diethyl azodicarboxylate (DEAD); Lv₁ is 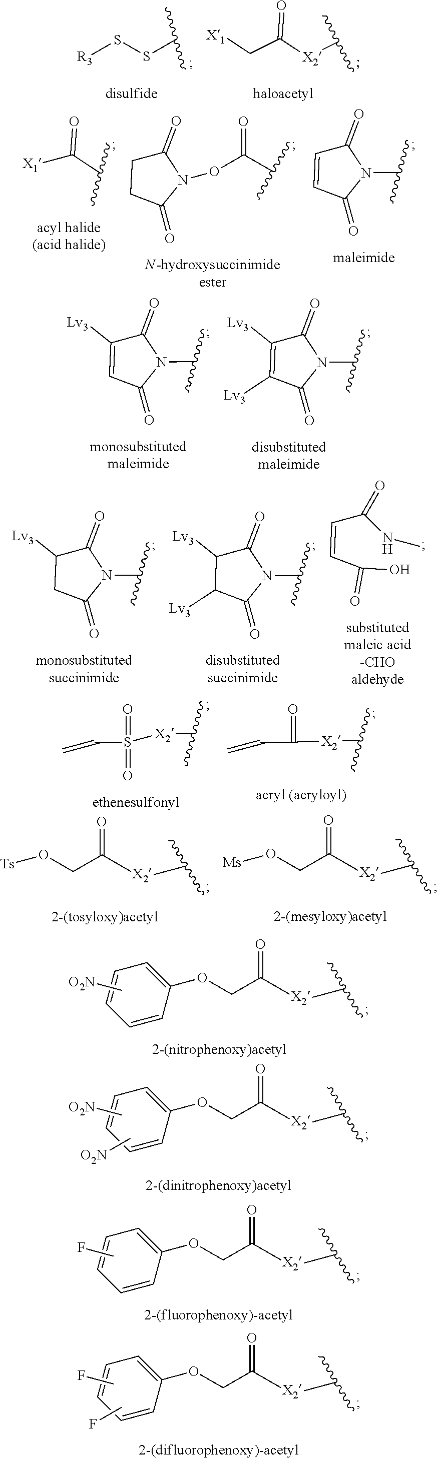 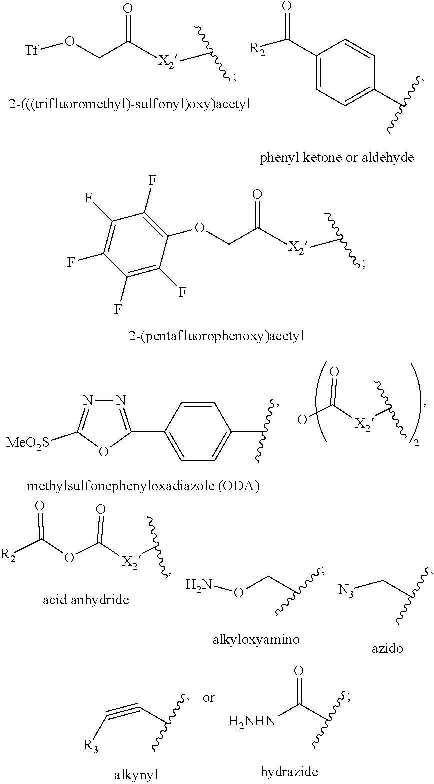
wherein X₁′ is F, Cl, Br, I or Lv₃; X₂′ is O, NH, N(R₁), or CH₂; R₃ is independently H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, an anhydride formed by acid itself, or formed with another anhydride of acetyl anhydride, or formyl anhydride; or an intermediate molecule generated with a condensation reagent for peptide coupling reactions or for Mitsunobu reactions.

25. A side chain-linkage compound of Formula (V) and (VI), which is capable of reacting with a cell-binding molecule T to form a conjugate: 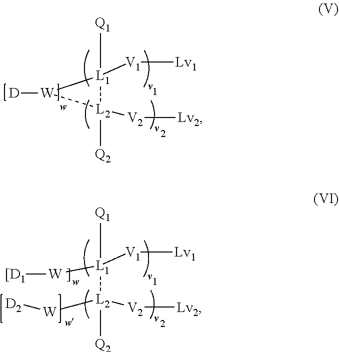
Wherein:
“ [symbol] ” represents a single bond; [symbol] is a single bond, double bond or absent; n is 1 to 30;
L₁ and L₂ are, the same or different, independently O, NH, N, S, P, NNH, NHNH, N(R₃), N(R₁₂), N(R₁₂)N(R_{12′}), CH, CO, C(O)NH, C(O)O, NHC(O)NH, NHC(O)O, polyethyleneoxy unit of formula (OCH₂CH₂)ₚOR₁₂, (OCH₂CH—(CH₃))ₚOR₁₂, NH(CH₂CH₂O)ₚR₁₂, NH(CH₂CH(CH₃)O)ₚR₁₂, N[(CH₂CH₂O)ₚR₁₂]—[(CH₂CH₂O)_{p′}R_{12′}], (OCH₂CH₂)ₚCOOR₁₂, or CH₂CH₂(OCH₂CH₂)ₚCOOR₁₂, wherein p and p′ are independently an integer selected from 0 to about 1000, or a combination of two or more of the above groups; C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or (Aa)ᵣ, r=1-12 (one to 12 amino acid units), which is composed from natural or unnatural amino acids, or the same or different sequences of dipeptide, tripeptide, tetrapeptide, pentapeptide, hexapeptide, heptapeptide, octapeptide, nonapeptide, decapeptide, undecapeptide or dodecapeptide unit;
W is a stretcher unit having C₁-C₁₈, and a self-immolative spacer, a peptidyl unit, a hydrazone, a disulfide, a thioether, an ester, or an amide bond;
w and w′ are independently 1 or 2 or 3;
V₁ and V₂ are independently a spacer unit selected from O, NH, S, C₁-C₈ alkyl, C₂-C₈ heteroalkyl, alkenyl, or alkynyl, C₃-C₈ aryl, heterocyclic, carbocyclic, cycloalkyl, alkylcycloalkyl, heterocycloalkyl, heteroaralkyl, heteroalkylcycloalkyl, or alkylcarbonyl, or (Aa)ᵣ, r=1-12 (1 to 12 amino acid units), which is composed from a natural or unnatural amino acid, or the same or different sequences of dipeptide, tripeptide, tetrapeptide, pentapeptide, hexapeptide, heptapeptide, octapeptide, nonapeptide, decapeptide, undecapeptide or dodecapeptide unit; or (CH₂CH₂O)ₚ, p is 0-1000;
v₁ and v₂ are independently 0, 1 or 2, but v₁ and v₂ are not 0 at the same time; when v₁ or v₂ is 0, it means one of side chain Q₁ or Q₂ fragment is absent;
Q₁ and Q₂ are independently represented by Formula (I-q1): 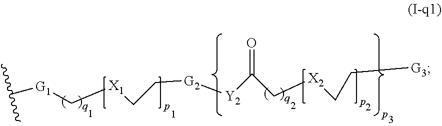
wherein “ [symbol] ” is a site linked to L₁ or L₂, G₁ and G₂ are independently OC(O), NHC(O), C(O), CH₂, NH, OC(O)NH, NHC(O)NH, O, S, B, P(O)(OH), NHP(O)(OH), NHP(O)(OH)NH, CH₂P(O)(OH)NH, OP(O)(OH)O, CH₂P(O)(OH)O, NHS(O)₂, NHS(O)₂NH, CH₂S(O)₂NH, OS(O)₂O, CH₂S(O)₂O, Ar, ArCH₂, ArO, ArNH, ArS, ArNR₁, or (Aa)_{q1}; G₃ is OH, SH, OR₁₂, SR₁₂, OC(O)R₁₂, NHC(O)R₁₂, C(O)R₁₂, CH₃, NH₂, NR₁₂, ⁺NH(R₁₂), ⁺N(R₁₂)(R_{12′}), C(O)OH, C(O)NH₂, NHC(O)NH₂, BH₂, BR₁₂R_{12′}, P(O)(OH)₂, NHP(O)(OH)₂, NHP(O)(NH₂)₂, S(O)₂(OH), (CH₂)_{q1}C(O)OH, (CH₂)_{q1}P(O)(OH)₂, C(O)(CH₂)_{q1}C(O)OH, OC(O)(CH₂)_{q1}C(O)OH, NHC(O)(CH₂)_{q1}C(O)OH, CO(CH₂)_{q1}P(O)(OH)₂, NHC(O)O(CH₂)_{q1}C(O)OH, OC(O)NH(CH₂)_{q1}C(O)OH, NHCO(CH₂)_{q1}—P(O)(OH)₂, NHC(O)(NH)(CH₂)_{q1}C(O)OH, CONH(CH₂)_{q1}P(O)(OH)₂, NHS(O)₂(CH₂)_{q1}C(O)OH, CO(CH₂)_{q1}S(O)₂(OH), NHS(O)₂NH(CH₂)_{q1}C(O)OH, OS(O)₂NH(CH₂)_{q1}C(O)OH, NHCO(CH₂)_{q1}S(O)₂(OH), NHP(O)(OH)(NH)(CH₂)_{q1}C(O)OH, CONH(CH₂)_{q1}S(O)(OH), OP(O)(OH)₂, (CH₂)_{q1}P(O)(NH)₂, NHS(O)₂(OH), NHS(O)₂NH₂, CH₂S(O)₂NH₂, OS(O)₂OH, OS(O)₂OR₁, CH₂S(O)₂OR₁₂, Ar, ArR₁₂, ArOH, ArNH₂, ArSH, ArNHR₁₂, or (Aa)_{q1}; (Aa)_{q1} is a peptide containing the same or different sequence of natural or unnatural amino acids; X₁ and X₂ are independently O, CH₂, S, S(O), NHNH, NH, N(R₁₂), ⁺NH(R₁₂), ⁺N(R₁₂)(R_{12′}), C(O), OC(O), OC(O)O, OC(O)NH, NHC(O)NH; Y₂ is O, NH, NR₁₂, CH₂, S, NHNH, or Ar; p₁, p₂ and p₃ are independently 0-100 but are not 0 at the same time; q₁ and q₂ are independently 0-24; R₁₂, R_{12′}, R₁₃ and R_{13′} are independently H, C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl;
or Q₁ and Q₂ are independently, linear or branched, a C₂-C₁₀₀ polycarboxylacid, a C₂-C₉₀ polyalkylamine, a C₆-C₉₀ oligosaachride or polysaccharide, a C₆-C₁₀₀ zwitterionic betaines or zwitterionic poly(sulfobetaine)) (PSB)s that consist of a quaternary ammonium cation and/or a sulfonate anion, a C₆-C₁₀₀ biodegradable polymer, composed of poly (lactic/glycolic acid) (PLGA), poly(acrylates), chitosan, copolymer of N-(2-hydroxypropyl)methacrylamide, poly[2-(methacryloyloxy)ethyl phosphorylcholine] (PMPC), poly-L-glutamic acid, poly(lactide-co-glycolide) (PLG), poly(lactide-co-glycolide), Poly(ethylene glycol) (PEG), poly(propylene glycol) (PPG), poly(lactide-co-glycolide), poly(ethylene glycol)-modified peptide, poly(ethylene glycol)-containing an amino acid or peptide, poly(ethylene glycol)-modified lipid, poly-glycine, poly-N-methyl-glycine, poly(ethylene glycol)-modified alkylcarboxic acid, poly(ethylene glycol)-modified alkylamine, poly(lactide-co-glycolide, hyaluronic acid (HA) (glycosaminoglycan), heparin/heparan sulfate (HSGAGs), chondroitin sulfate/dermatan sulfate (CSGAGs), poly(ethylene glycol)-modified alkylsulfate, poly(ethylene glycol)-modified alkylphosphate, or poly(ethylene glycol)-modified alkyl quaternary ammonium;
or any one or more of W, Q₁, Q₂, L₁, L₂, V₁, or V₂ can be independently absent but Q₁ and Q₂ are not absent at the same time;
D, D₁ and D₂ are independently a cytotoxic agent that is selected from calicheamicins, maytansinoids, camptothecins, taxanes, anthracyclines (daunorubicin/doxorubicin), vinca alkaloids, auristatins, eribulins, (pyrrolo)benzodiazepines (PBDs), CC-106/duocarmycins, tubulysins, amatoxins (such as amanitins), protein kinase inhibitors, MEK inhibitors, KSP inhibitors, nicotinamide phosphoribosyltransferase (NAMPT) inhibitors, immunotoxins, and analogs or prodrugs of these compounds;
Lv₁ and Lv₂ are independently a reacting group that can be reacted with a thiol, amine, carboxylic acid, selenol, phenol or hydroxyl group on a cell-binding molecule; Lv₁ is OH; F; Cl; Br; I; nitrophenol; N-hydroxysuccinimide (NHS); phenol; dinitrophenol; pentafluorophenol; tetrafluorophenol; difluorophenol; mono-fluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, anhydride formed by acid themselves, or formed with another C₁-C₈ acid anhydride; or an intermediate molecule generated with a condensation reagent for peptide coupling reactions, or for Mitsunobu reactions; the condensation reagent is selected from the group consisting of EDC (N-(3-Dimethylaminopropyl)-N′-ethylcarbodiimide), DCC (Dicyclohexyl-carbodiimide), N,N′-Diisopropylcarbodiimide (DIC), N-Cyclohexyl-N′-(2-morpholino-ethyl)carbodiimide metho-p-toluenesulfonate (CMC, or CME-CDI), 1,1′-Carbonyldiimidazole (CDI), TBTU (0-(Benzotriazol-1-yl)-N,N,N′,N′-tetramethyluronium tetrafluoroborate), N,N,N′,N′-Tetramethyl-O-(1H-benzo-triazol-1-yl)-uronium hexafluoro-phosphate (HBTU), (Benzotriazol-1-yloxy)tris(dimethylamino)-phosphonium hexafluorophosphate (BOP), (Benzotriazol-1-yloxy)tripyrrolidinophosphonium hexafluorophosphate (PyBOP), Diethyl cyanophosphonate (DEPC), Chloro-N,N,N′,N′-tetra-methylformamidiniumhexafluorophosphate, 1-[Bis(dimethyl-amino)methylene]-1H-1,2,3-triazolo[4,5-b]pyridinium 3-oxid hexafluorophosphate (HATU), 1-[(Dimethylamino)-(morpholino)methylene]-1H-[1,2,3]triazolo[4,5-b]pyridine-1-ium 3-oxide hexafluoro-phosphate (HDMA), 2-Chloro-1,3-dimethyl-imidazolidinium hexafluorophosphate (CIP), Chlorotripyrrolidinophosphonium hexafluorophosphate (PyCloP), Fluoro-N,N,N′,N′-bis(tetra-methylene)-formamidinium hexafluorophosphate (BTFFH), N,N,N′,N′-Tetramethyl-S-(1-oxido-2-pyridyl)-thiuronium hexafluorophosphate, O-(2-Oxo-1(2H)pyridyl)-N,N,N′,N′-tetramethyluronium tetrafluoroborate (TPTU), S-(1-Oxido-2-pyridyl)-N,N,N′,N′-tetramethylthiuronium tetrafluoroborate, O-[(Ethoxycarbonyl)-cyanomethylenamino]-N,N,N′,N′-tetramethyluronium hexafluorophosphate (HOTU), (1-Cyano-2-ethoxy-2-oxoethylidenaminooxy) dimethylamino-morpholino-carbenium hexafluorophosphate (COMU), 0-(Benzotriazol-1-yl)-N,N,N′,N′-bis(tetramethylene)-uronium hexafluorophosphate (HBPyU), N-Benzyl-N′-cyclohexyl-carbodiimide (with, or without polymer-bound), Dipyrrolidino(N-succinimidyl-oxy)carbenium hexafluoro-phosphate (HSPyU), Chlorodipyrrolidinocarbenium hexafluorophosphate (PyClU), 2-Chloro-1,3-dimethylimidazolidinium tetrafluoroborate (CIB), (Benzotriazol-1-yloxy)dipiperidino-carbenium hexafluorophosphate (HBPipU), O-(6-Chlorobenzotriazol-1-yl)-N,N,N′,N′-tetramethyluronium tetrafluoroborate (TCTU), Bromotris(dimethylamino)-phosphonium hexafluorophosphate (BroP), Propylphosphonic anhydride (PPACA, T3P©), 2-Morpholinoethyl isocyanide (MEI), N,N,N′,N′-Tetramethyl-O—(N-succinimidyl)uronium hexafluorophosphate (HSTU), 2-Bromo-1-ethyl-pyridinium tetrafluoroborate (BEP), O-[(Ethoxycarbonyl)cyano-methylenamino]-N,N,N′,N′-tetra-methyluronium tetrafluoroborate (TOTU), 4-(4,6-Dimethoxy-1,3,5-triazin-2-yl)-4-methylmorpholiniumchloride (MMTM, DMTMM), N,N,N′,N′-Tetramethyl-O—(N-succinimidyl)uronium tetrafluoroborate (TSTU), O-(3,4-Dihydro-4-oxo-1,2,3-benzotriazin-3-yl)-N,N,N′,N′-tetramethyluronium tetrafluoro-borate (TDBTU), 1,1′-(Azodicarbonyl)-dipiperidine (ADD), Di-(4-chlorobenzyl)azodi-carboxylate (DCAD), Di-tert-butyl azodicarboxylate (DBAD), Diisopropyl azodicarboxylate (DIAD), and Diethyl azodicarboxylate (DEAD); Lv₁ and Lv₂ are independently 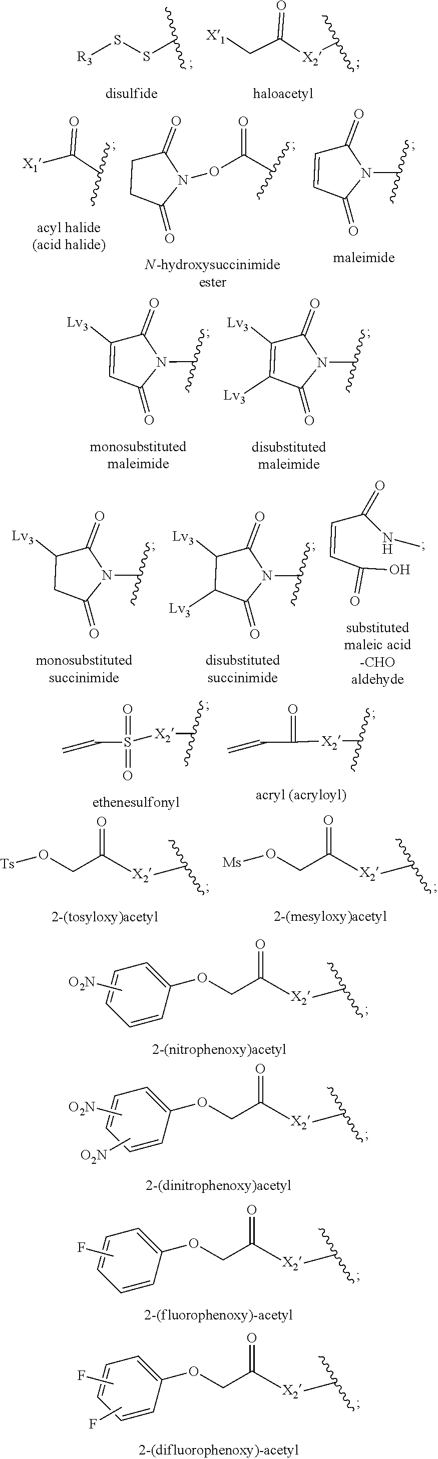 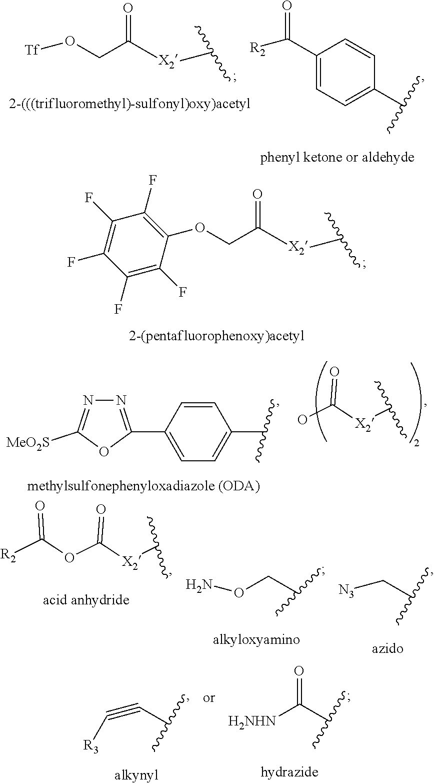
wherein X₁′ is F, Cl, Br, I or Lv₃; X₂′ is O, NH, N(R₁), or CH₂; R₃ is independently H, aromatic, heteroaromatic, or aromatic group wherein one or several H atoms are replaced independently by —R₁, -halogen, —OR₁, —SR₁, —NR₁R₂, —NO₂, —S(O)R₁, —S(O)₂R₁, or —COOR₁; Lv₃ is a leaving group selected from F, Cl, Br, I, nitrophenol; N-hydroxysuccinimide (NHS); phenol; dinitrophenol; pentafluorophenol; tetrafluorophenol; difluorophenol; monofluorophenol; pentachlorophenol; triflate; imidazole; dichlorophenol; tetrachlorophenol; 1-hydroxybenzotriazole; tosylate; mesylate; 2-ethyl-5-phenylisoxazolium-3′-sulfonate, an anhydride formed by acid itself, or formed with another anhydride of acetyl anhydride, or formyl anhydride; or an intermediate molecule generated with a condensation reagent for peptide coupling reactions or for Mitsunobu reactions.

26. The side chain-linked conjugate compound of claim 22, wherein the side chain Q₁ and Q₂ are independently selected from Iq-01 to Iq-36 below: 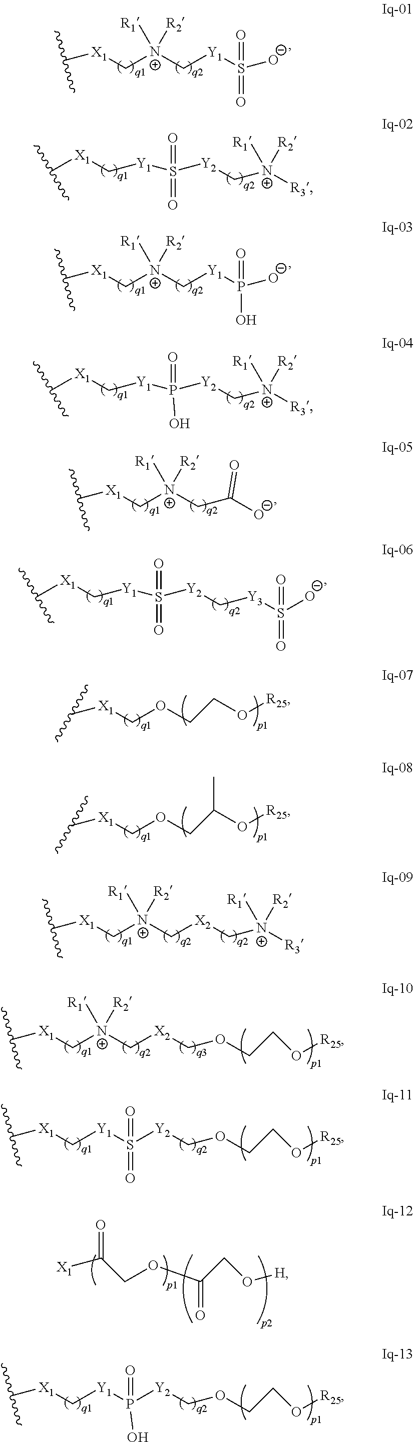 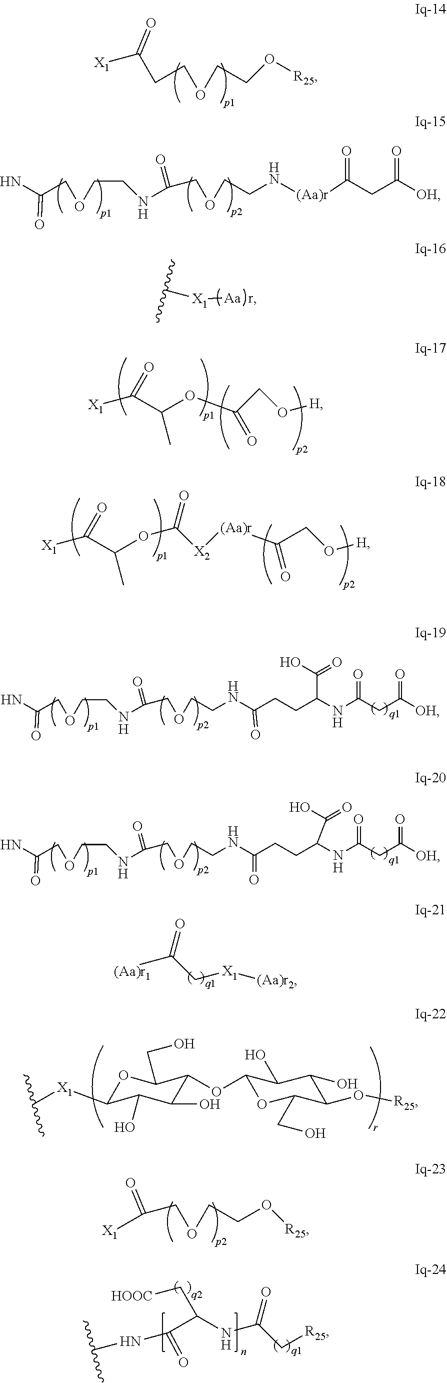 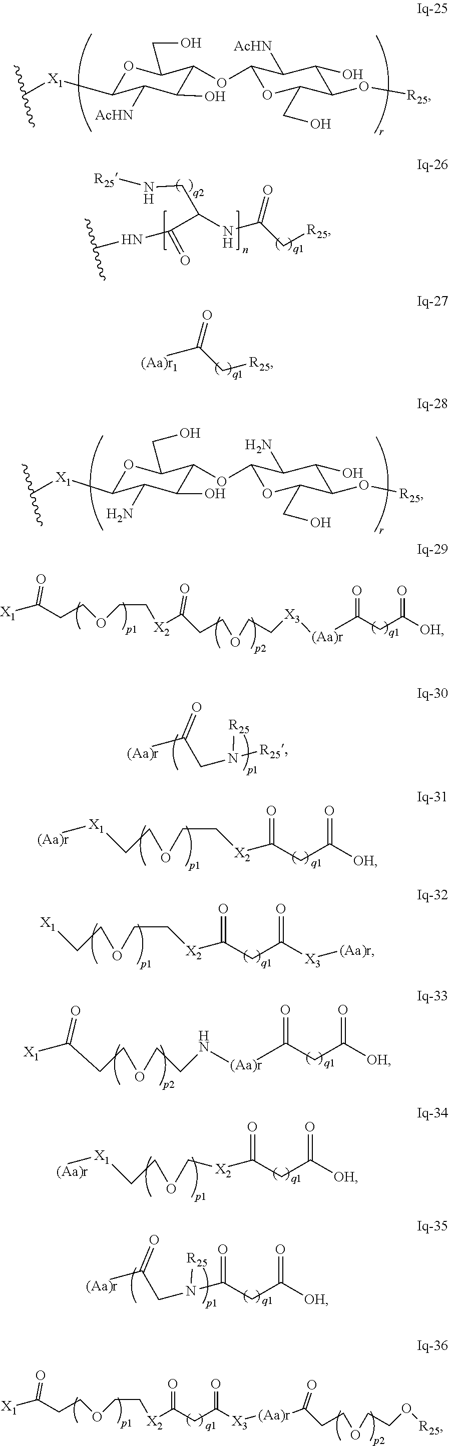 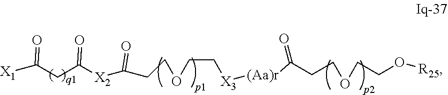
wherein R₂₅ and R_{25′} are independently H; HC(O), CH₃C(O), CH₃C(NH), NHCH₃, COOH, CONH₂, CONHCH₃, C₁-C₁₈ alkyl, C₁-C₁₈ alkyl, alkyl-Y₁—SO₃H, C₁-C₁₈ alkyl-Y₁—PO₃H₂, C₁-C₁₈ alkyl-Y₁—CO₂H, C₁-C₁₈ alkyl-Y₁—N⁺R₁₂R₁₃R₁₃′R₁₄, C₁-C₁₈ alkyl-Y₁—CONH₂, C₂-C₁₈ alkylene, C₂-C₁₈ ester, C₂-C₁₈ ether, C₂-C₁₈ amine, C₂-C₁₈ alkyl carboxylamide, C₃-C₁₈ Aryl, C₃-C₁₈ cyclic alkyl, C₃-C₁₈ hyterocyclic, 1-24 amino acids; C₂-C₁₈ lipid, a C₂-C₁₈ fatty acid or a C₂-C₁₈ fatty ammonium lipid; X₁ and X₂ are independently NH, N(R₁₂′), O, CH₂, S, C(O), S(O), S(O₂), P(O)(OH), NHNH, CH═CH, Ar or (Aa)q₁, q₁=0-24 (0-24 amino acids, q₁=0 means absent); X₃, X₄, Y₁, Y₂ and Y₃ are independently NH, N(R₁₂′), O, C(O), CH₂, S, S(O), NHNH, C(O), OC(O), OC(O)O, OC(O)NH, NHC(O)NH, Ar or (Aa)q₁, X₁, X₂, X₃, X₄, Y₁, Y₂ and Y₃ can be independently absent; p₁, p₂ and p₃ are independently 0-100 but are not 0 at the same time; q₁, q₂ and q₃ are independently 0-24; R₁₂, R₁₃, R₁₃′ and R₁₄′ are independently H or C₁-C₆ alkyl; Aa is natural or unnatural amino acid; Ar or (Aa)q₁, is the same or different sequence of peptide; q₁=0 means (Aa)q₁ absent.

27. The compound according to claim 22, wherein D is one selected from the following formulae:
(A) a Calicheamicin analog: 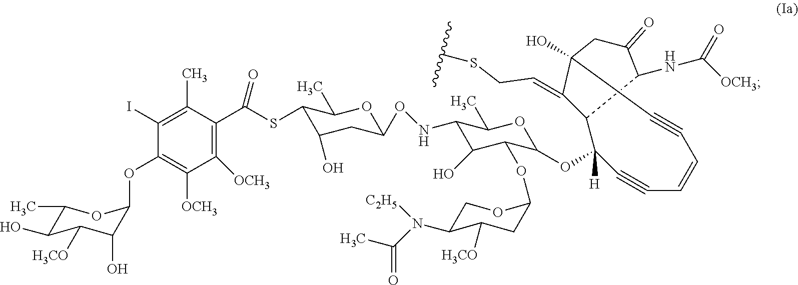
wherein [symbol] is a connecting site;
(B) a Maytansinoid: 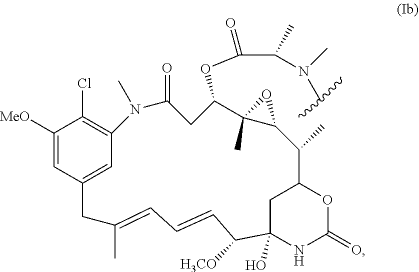
wherein [symbol] is a connecting site;
(C) a Camptothecin (CPTs) and its derivatives: 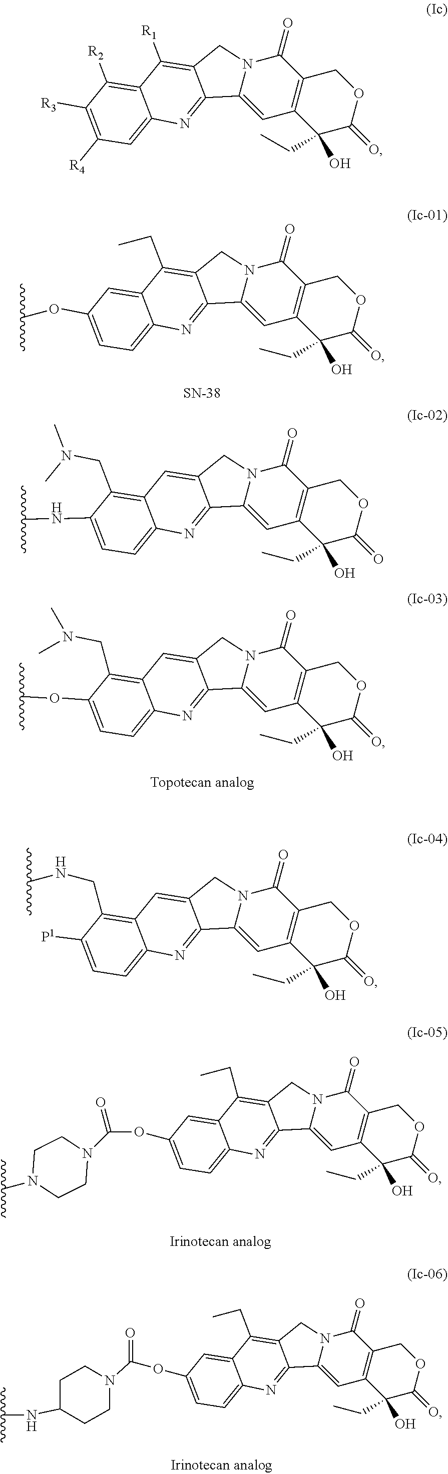 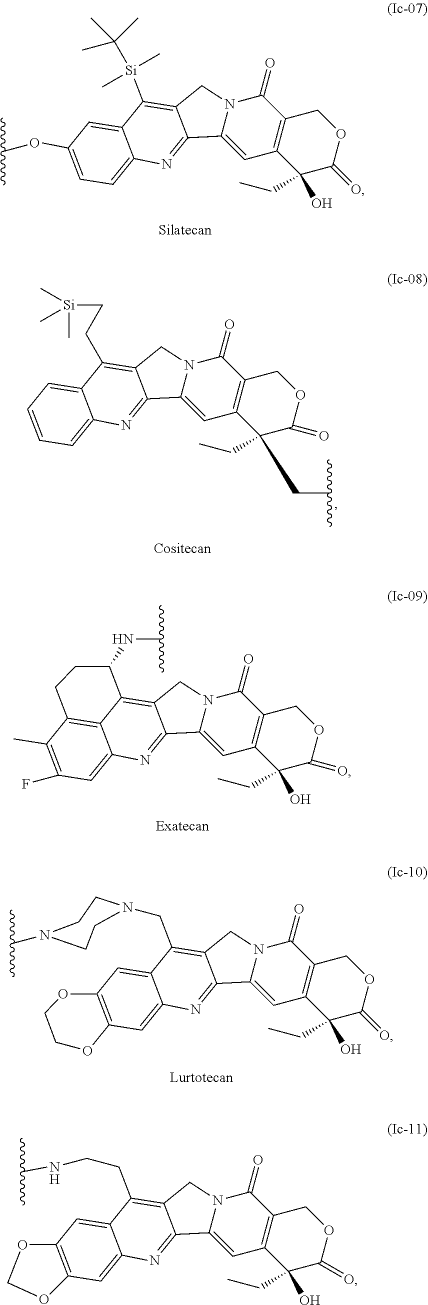 [SMILES: *Oc1ccc2nc3c(c([Si](C)(C)C(C)(C)C)c2c1)Cn1c-3cc2c(c1=O)COC(=O)[C@]2(O)CC ; *C[C@]1(CC)C(=O)OCc2c1cc1n(c2=O)Cc2c-1nc1ccccc1c2CC[Si](C)(C)C ; *N[C@H]1CCc2c(C)c(F)cc3nc4c(c1c23)Cn1c-4cc2c(c1=O)COC(=O)[C@]2(O)CC ; *N1CCN(Cc2c3c(nc4cc5c(cc24)OCCO5)-c2cc4c(c(=O)n2C3)COC(=O)[C@]4(O)CC)CC1 ; *NCCc1c2c(nc3cc4c(cc13)OCO4)-c1cc3c(c(=O)n1C2)COC(=O)[C@]3(O)CC] 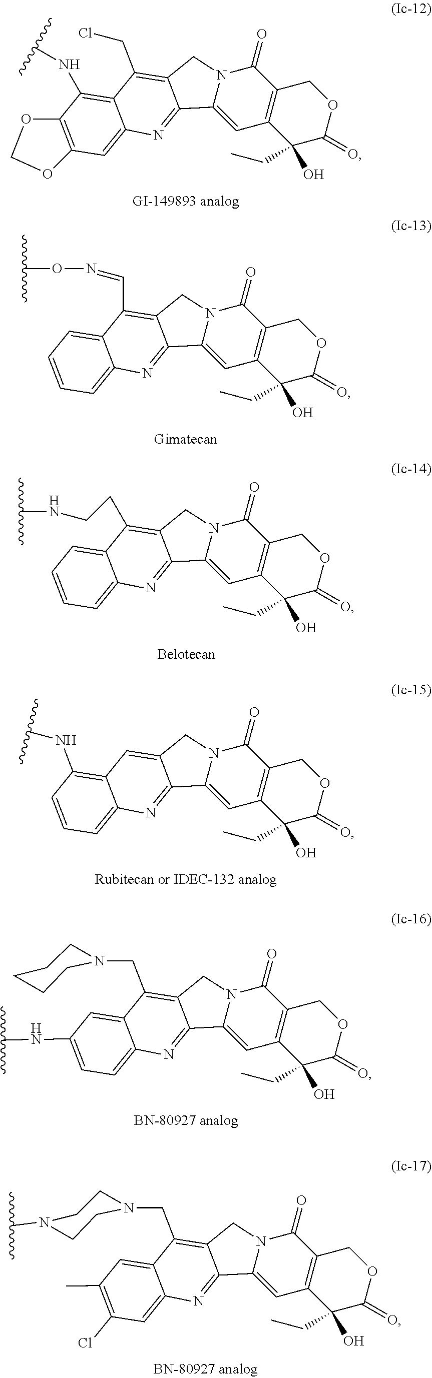 [SMILES: *Nc1c2c(cc3nc4c(c(CCl)c13)Cn1c-4cc3c(c1=O)COC(=O)[C@]3(O)CC)OCO2 ; *O/N=C\c1c2c(nc3ccccc13)-c1cc3c(c(=O)n1C2)COC(=O)[C@]3(O)CC ; *NCCc1c2c(nc3ccccc13)-c1cc3c(c(=O)n1C2)COC(=O)[C@]3(O)CC ; *Nc1cccc2nc3c(cc12)Cn1c-3cc2c(c1=O)COC(=O)[C@]2(O)CC ; *Nc1ccc2nc3c(c(CN4CCCCC4)c2c1)Cn1c-3cc2c(c1=O)COC(=O)[C@]2(O)CC ; *N1CCN(Cc2c3c(nc4cc(Cl)c(C)cc24)-c2cc4c(c(=O)n2C3)COC(=O)[C@]4(O)CC)CC1]
or a derivative thereof with one or more isotopes, or a pharmaceutically acceptable salt, hydrate, or hydrated salt thereof; or a polymorphic crystalline structure thereof; or an optical isomer, racemate, diastereomer or enantiomer thereof; wherein [symbol] is a connecting site;
wherein R₁, R₂ and R₄ are independently selected from H, F, Cl, Br, CN, NO₂, C₁-C₈ alkyl; O—C₁-C₈ alkyl; NH—C₁-C₈ alkyl; C₂-C₈ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₂-C₈ ester, ether, amide, carbonate, urea, or carbamate; R3 is H, OH, NH₂, C₁-C₈ alkyl; O—C₁-C₈ alkyl; NH—C₁-C₈ alkyl; C₂-C₈ heteroalkyl, alkylcycloalkyl, or heterocycloalkyl; or C₂-C₈ ester, ether, amide, carbonate, urea, or carbamate; or R₁ and R₂, R₂ and R₃ or R₃ and R₄ independently form a 5-7 membered carbocyclic, heterocyclic, heterocycloalkyl, aromatic or heteroaromatic ring system; P¹ is H, OH, NH₂, COOH, C(O)NH₂, OCH₂OP(O)(OR¹⁸)₂, OC(O)OP(O)(OR¹⁸)₂, OPO(OR¹⁸)₂, NHPO(OR¹⁸)₂, OC(O)R¹⁸, O P(O)(OR¹⁸)OP(O)(OR¹⁸)₂, OC(O)NHR¹⁸, OC(O)N(C₂H₄)₂NCH₃, OSO₂(OR¹⁸), O—(C₄-C₁₂-glycoside), OC(O)N(C₂H₄)₂CH₂N(C₂H₄)₂CH₃, C₁-C₈ linear or branched alkyl or heteroalkyl; C₂-C₈ linear or branched alkenyl, alkynyl, alkylcycloalkyl, or heterocycloalkyl; C₃-C₈ linear or branched aryl, Ar-alkyl, heterocyclic, carbocyclic, cycloalkyl, heteroalkylcycloalkyl, alkylcarbonyl, or heteroaryl; carbonate (—C(O)OR¹⁷), carbamate (—C(O)NR¹⁷R¹⁸); R¹⁷ and R¹⁸ are independently H, C₁-C₈ linear or branched alkyl or heteroalkyl; C₂-C₈ linear or branched alkenyl, alkynyl, alkylcycloalkyl, or heterocycloalkyl; C₃-C₈ linear or branched aryl, Ar-alkyl, heterocyclic, carbocyclic, cycloalkyl, heteroalkylcycloalkyl, alkylcarbonyl, or heteroaryl; carbonate (—C(O)OR¹⁷), or carbamate (—C(O)NR¹⁷R¹⁸);
(D) a Taxane and its analogs: 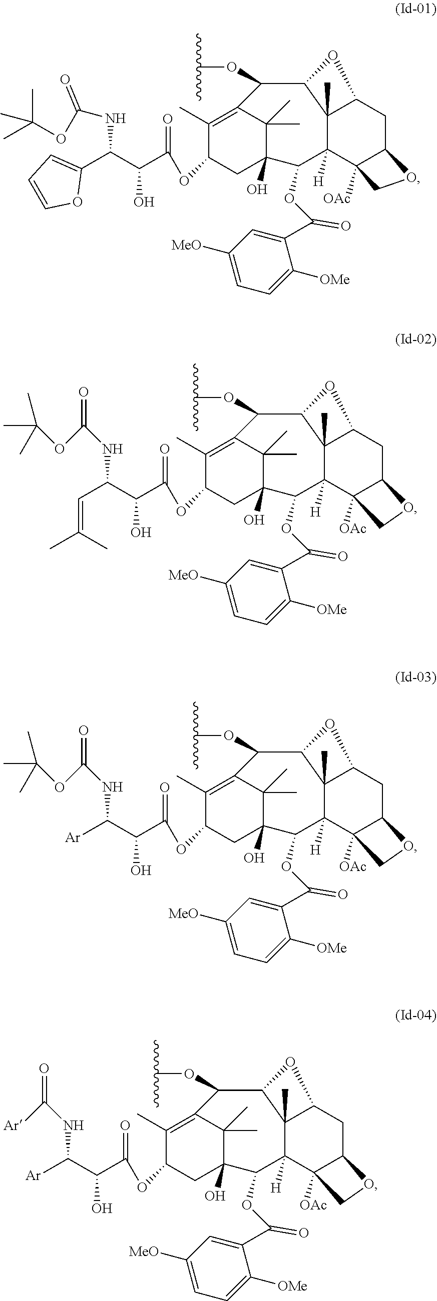
wherein [symbol] is a connecting site; Ar and Ar′ are independently aryl or heteroaryl;
(E) an Anthracycline and its analog: 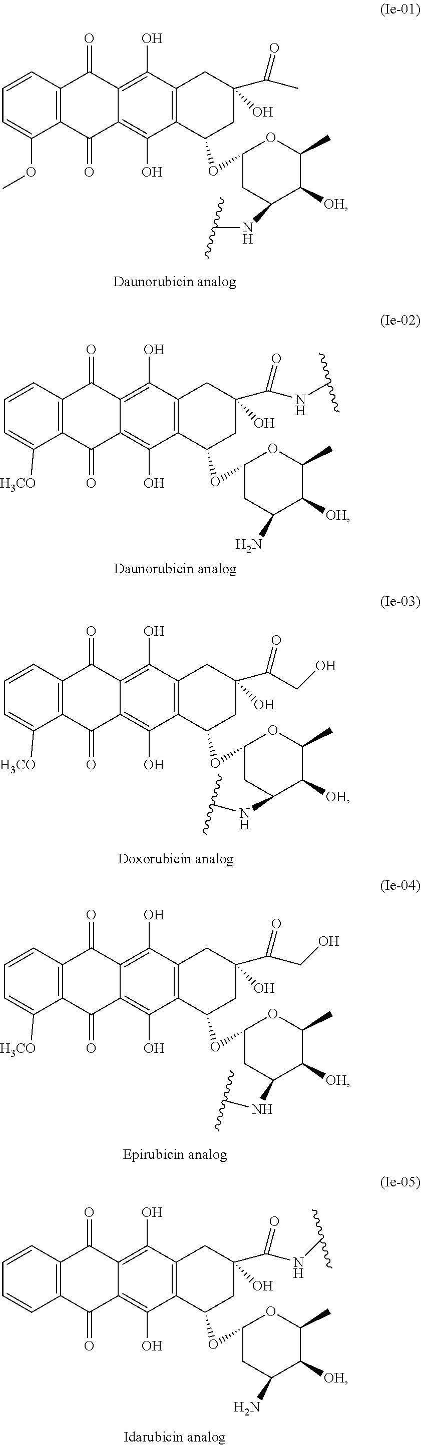 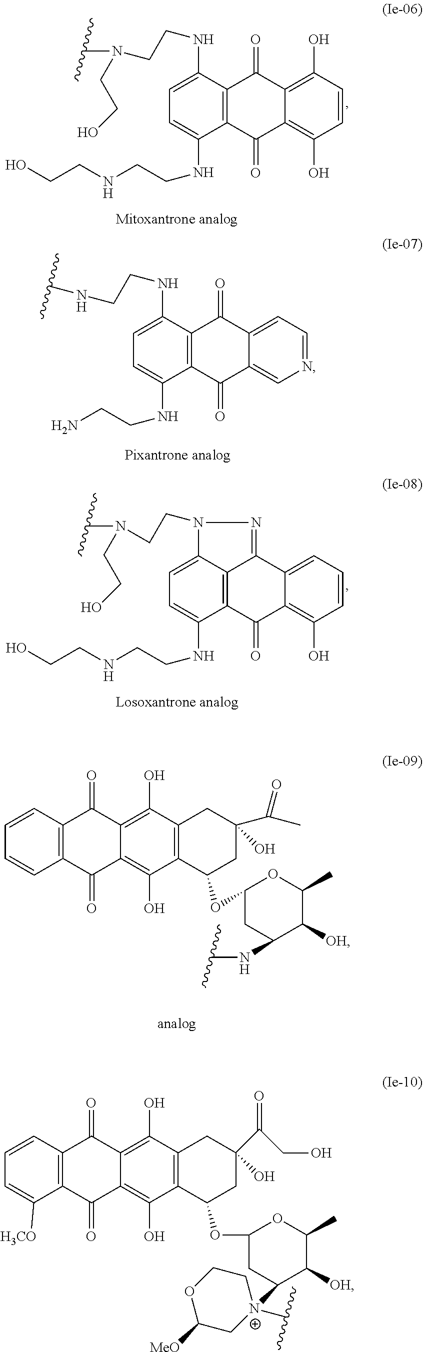 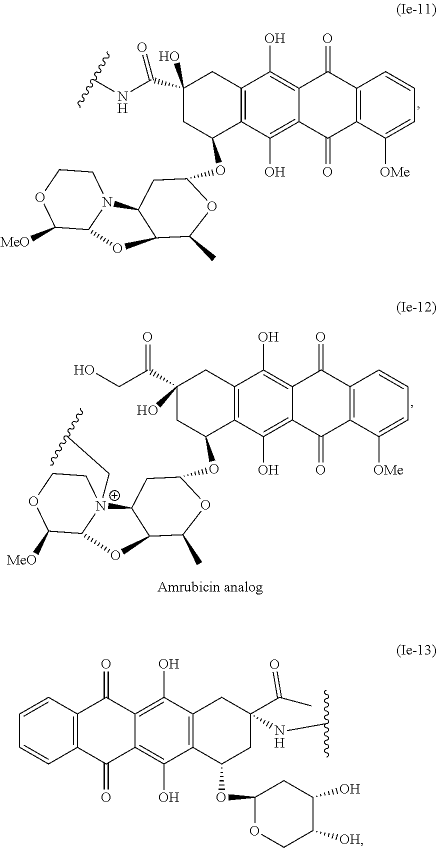
wherein [symbol] is a connecting site;
(F) a Vinca alkaloid: 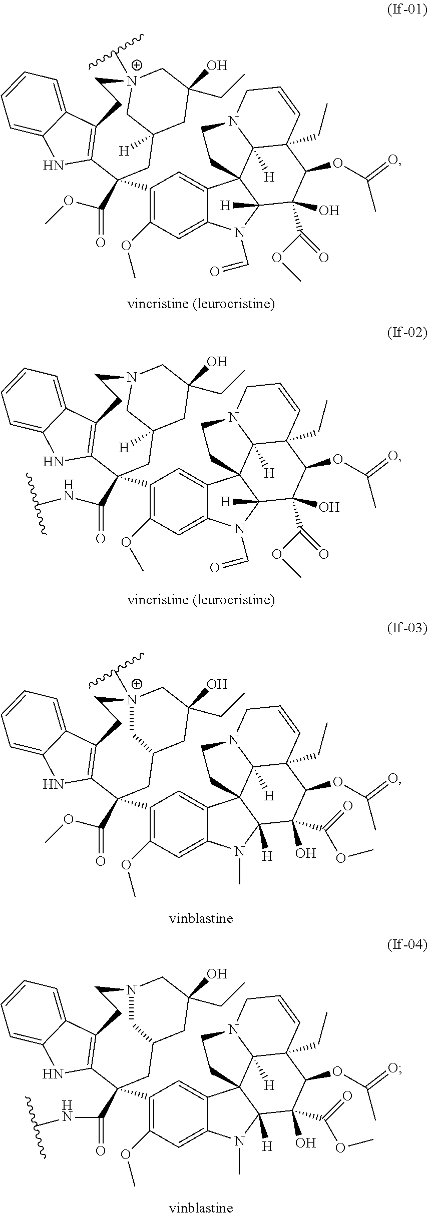 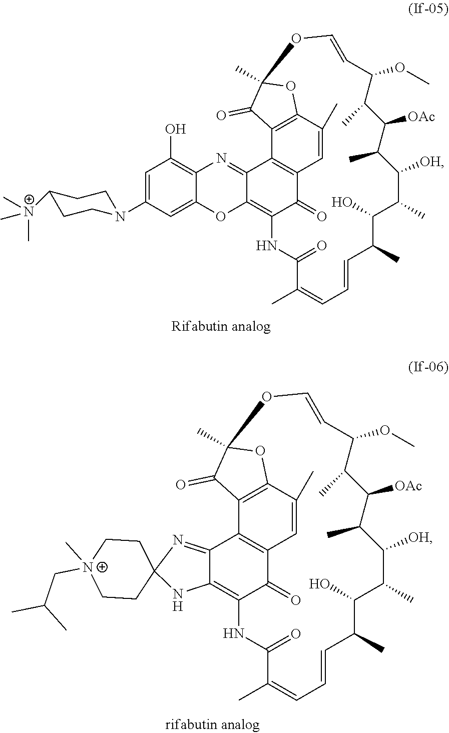
(G) an Auristatin or dolastatin analog: 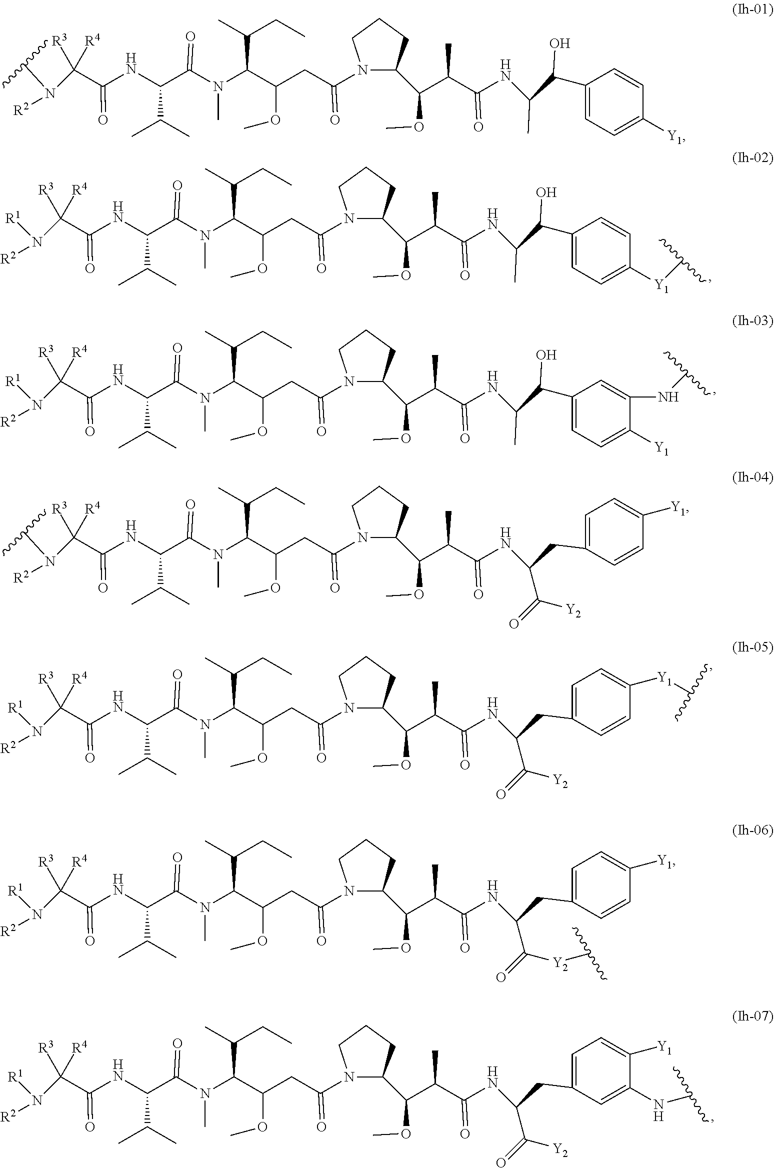
or a derivative thereof with one or more isotopes, or a pharmaceutically acceptable salt, hydrate, or hydrated salt thereof; or a polymorphic crystalline structure thereof; or an optical isomer, racemate, diastereomer or enantiomer thereof;
wherein R¹, R², R³, R⁴ and R⁵ are independently H; C₁-C₈ linear or branched alkyl, aryl, heteroaryl, heteroalkyl, alkylcycloalkyl, ester, ether, amide, amine, heterocycloalkyl, or acyloxylamine; or peptide containing 1-8 aminoacids, or polyethyleneoxy unit having formula (OCH₂CH₂)ₚ or (OCH₂CH(CH₃))ₚ, wherein p is an integer from 1 to about 5000; or two Rs: R¹ and R², R² and R³, R¹ and R³ or R³ and R⁴ can form a 3-8 member cyclic ring of alkyl, aryl, heteroaryl, heteroalkyl, or alkylcycloalkyl group; X₃ is H, CH₃ or X₁′R₁′, wherein X₁′ is NH, N(CH₃), NHNH, O, or S, and R₁′ is H or C₁-C₈ linear or branched alkyl, aryl, heteroaryl, heteroalkyl, alkylcycloalkyl, or acyloxylamines; R₃′ is H or C₁-C₆ linear or branched alkyl; Z₃′ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, or O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, or NR₁R₂R₃; Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) or C(O)NR₁ when linked to a connecting site “ [symbol] ”; or OH, NH₂, NHNH₂, NHR₅, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH or C(O)NHR₁ when not linked to the connecting site “ [symbol] ”; R₁₂ is OH, NH₂, NHR₁, NHNH₂, NHNCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R¹′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NH—SO₃H, NH(CH₂CH₂O)ₚCH₂CH₂NHSO₃H, R₁—NHSO₃H, NH—R₁—NHSO₃H, O(CH₂CH₂O)ₚCH₂—CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂—CH₂NH₂, NH(CH₂CH₂S)ₚCH₂CH₂NH₂, NH(CH₂CH₂NH)ₚCH₂CH₂OH, NH(CH₂CH₂S)ₚCH₂—CH₂OH, NH—R₁—NH₂, or NH(CH₂CH₂O)ₚCH₂CH₂NHPO₃H₂, wherein Aa is 1-8 the same or different aminoacids; p is 1-5000; R₁, R₂, R₃, R₄, R₅, R₅′, Z₁, Z₂, and n are defined the same above;
(H) Eribulin: 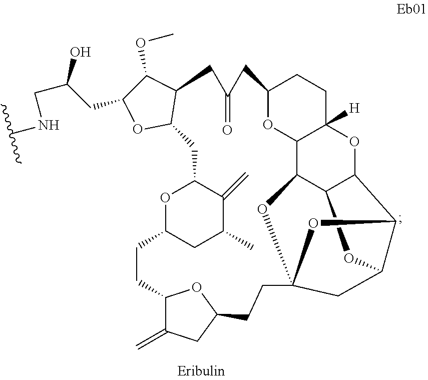 [SMILES: *NC[C@@H](O)C[C@H]1O[C@H]2C[C@H]3O[C@@H](CC[C@@H]4O[C@@H](CC[C@@]56C[C@H]7O[C@@H]8C(O[C@H]9CC[C@H](CC(=O)C[C@@H]2[C@H]1OC)OC9[C@@H]8O5)[C@H]7O6)CC4=C)C[C@@H](C)C3=C]
(I) an Inhibitor of nicotinamide phosphoribosyltransferases (NAMPT): 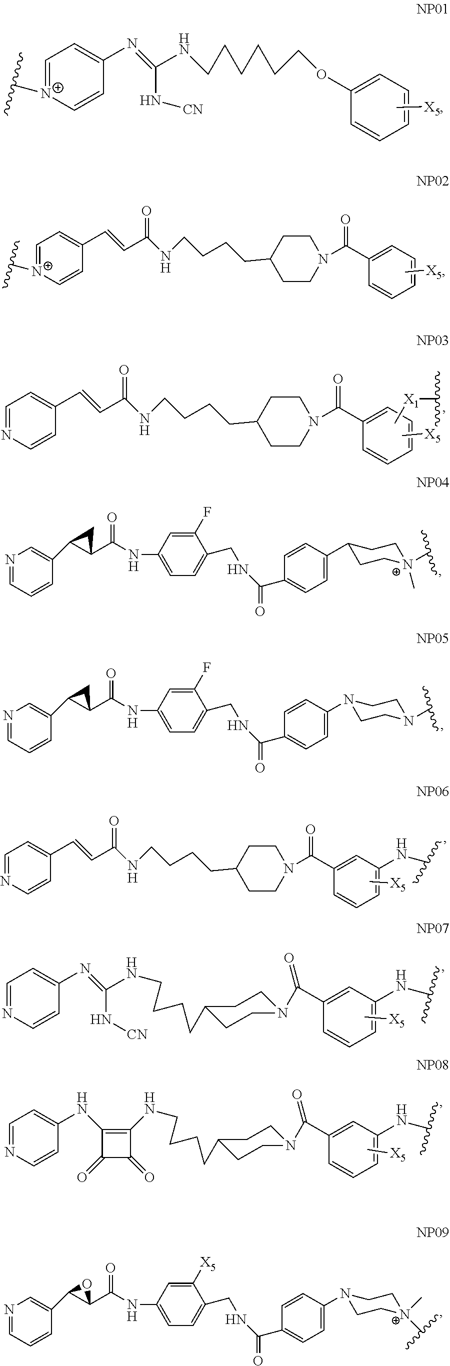
or a derivative thereof with one or more isotopes, or a pharmaceutically acceptable salt, hydrate, or hydrated salt thereof; or a polymorphic crystalline structure thereof; or an optical isomer, racemate, diastereomer or enantiomer thereof; wherein “ [symbol] ” and X₁ are defined the same above; X₅ is F, Cl, Br, I, OH, OR₁, R₁, OPO₃H₂, OSO₃H, NHR₁, OCOR₁, or NHCOR₁;
(J) a benzodiazepine dimer and its analog: 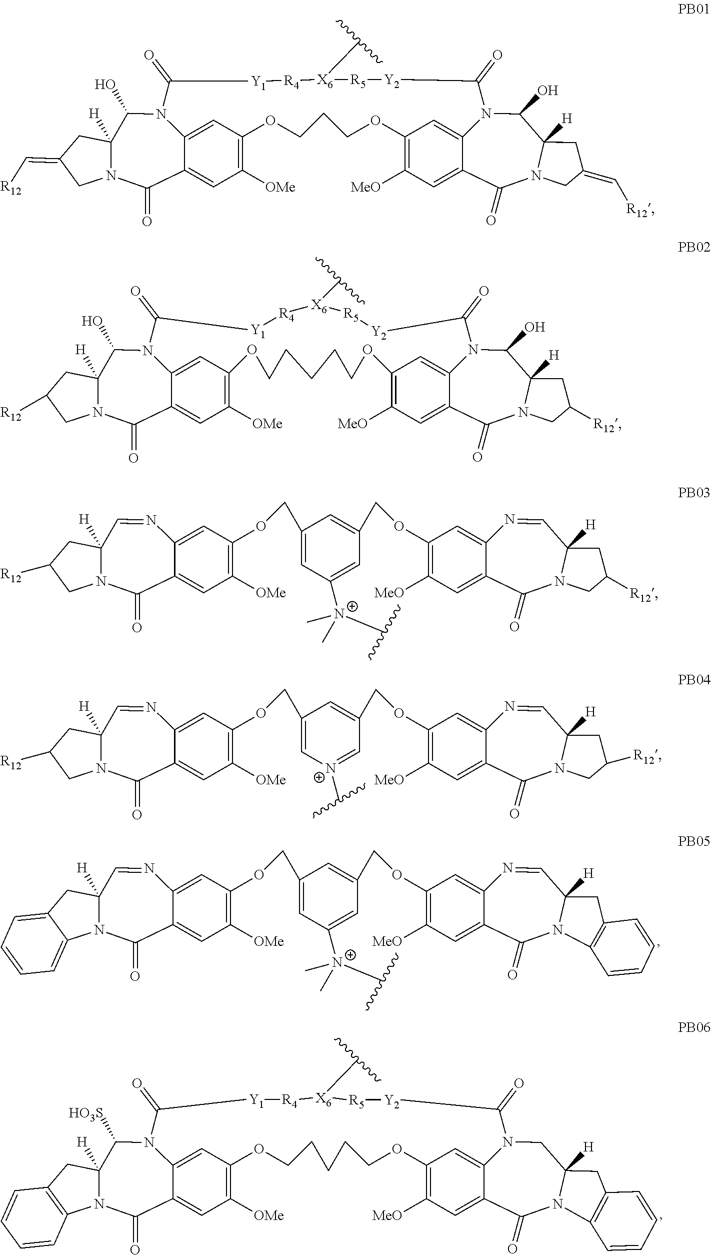 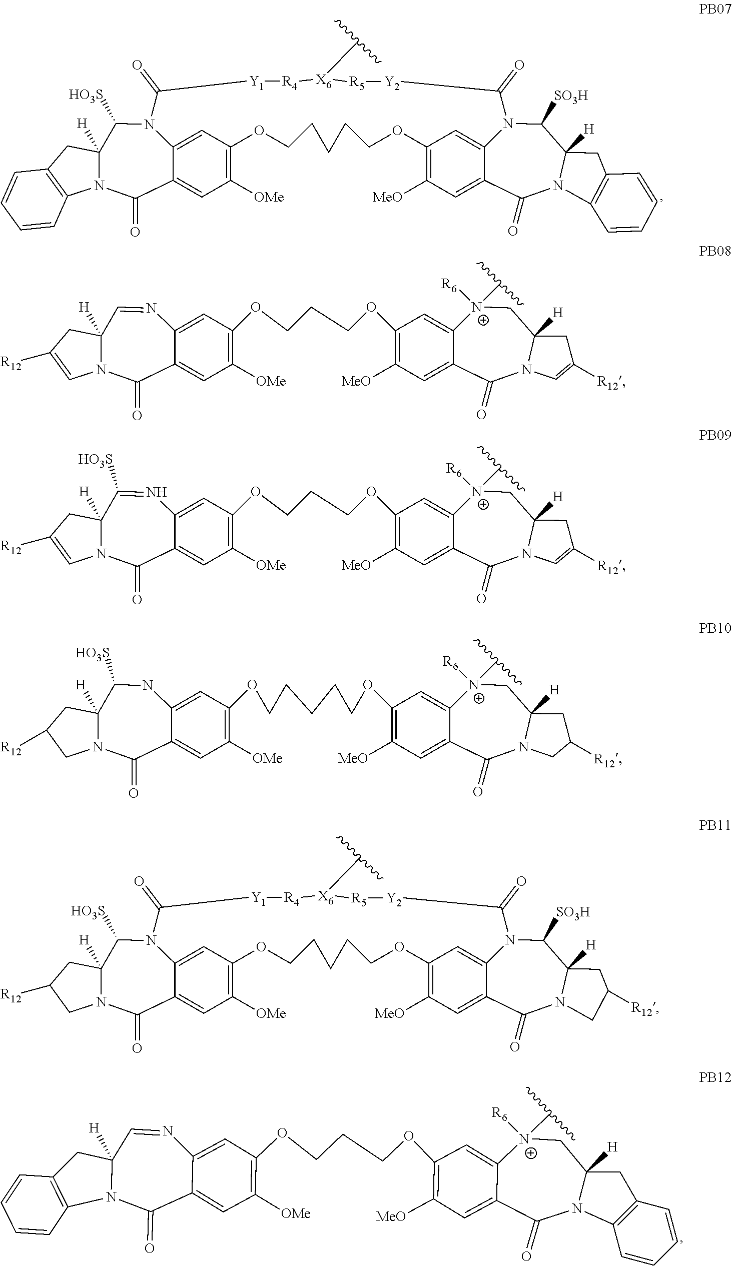 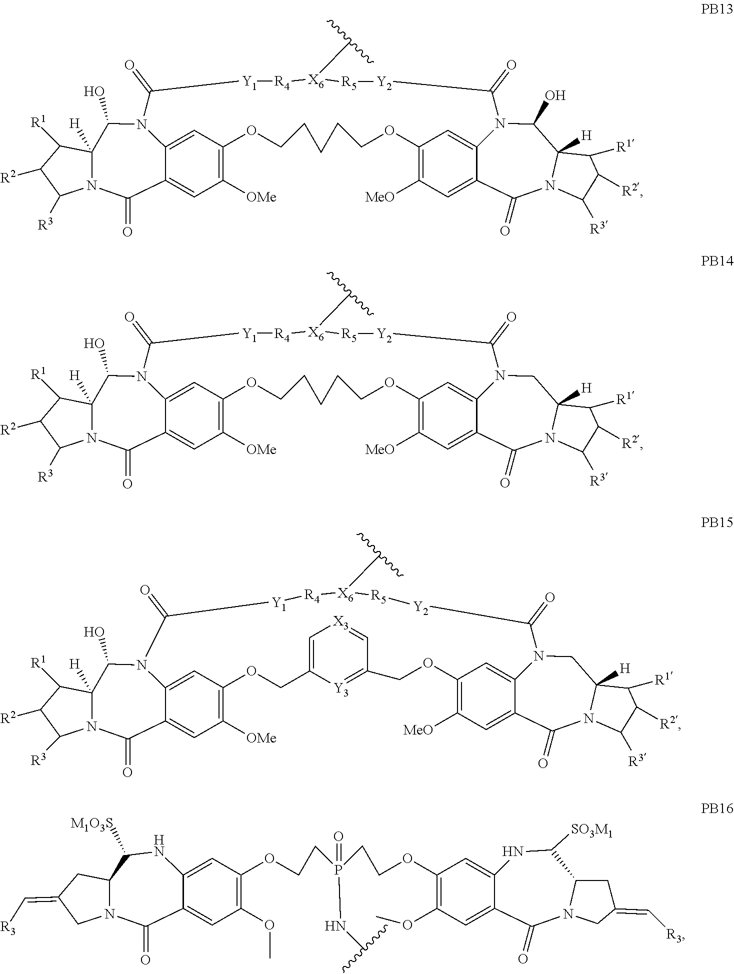
or a derivative thereof with one or more isotopes, or a pharmaceutically acceptable salt, hydrate, or hydrated salt thereof; or a polymorphic crystalline structure thereof; or an optical isomer, racemate, diastereomer or enantiomer thereof; wherein X₁, X₂, Y₁ and Y₂ are independently O, N, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; R¹, R², R³, R^{1′}, R^{2′}, and R^{3′} are independently H; F; Cl; ═O; ═S; OH; SH; C₁-C₈ linear or branched alkyl, aryl, alkenyl, heteroaryl, heteroalkyl, alkylcycloalkyl, ester (COOR₅ or —OC(O)R₅), ether (OR₅), amide (CONR₅), carbamate (OCONR₅), amine (NHR₅, NR₅R₅′), heterocycloalkyl, or acyloxylamine (—C(O)NHOH, —ONHC(O)R₅); or peptide containing 1-20 natural or unnatural aminoacids, or polyethyleneoxy unit of formula (OCH₂CH₂)ₚ or (OCH₂CH(CH₃))ₚ, wherein p is an integer from 1 to about 5000; or two Rs: R¹ and R², R² and R³, R¹ and R³, R^{1′} and R^{2′}, R^{2′} and R^{3′}, or R^{1′} and R^{3′} can independently form a 3-8 member cyclic ring of alkyl, aryl, heteroaryl, heteroalkyl, or alkylcycloalkyl group; X₃ and Y₃ are independently N, NH, CH₂ or CR₅, wherein R₄, R₅, R₆, R₁₂ and R₁₂′ are independently H, OH, NH₂, NH(CH₃), NHNH₂, COOH, SH, OZ₃, SZ₃, F, Cl, or C₁-C₈ linear or branched alkyl, aryl, heteroaryl, heteroalkyl, alkylcycloalkyl, or acyloxylamines; Z₃ is H, OP(O)(OM₁)(OM₂), OCH₂OP(O)(OM₁)(OM₂), OSO₃M₁, or O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, or NR₁R₂R₃;
(K) an CC-1065 analog and doucarmycin analogs: 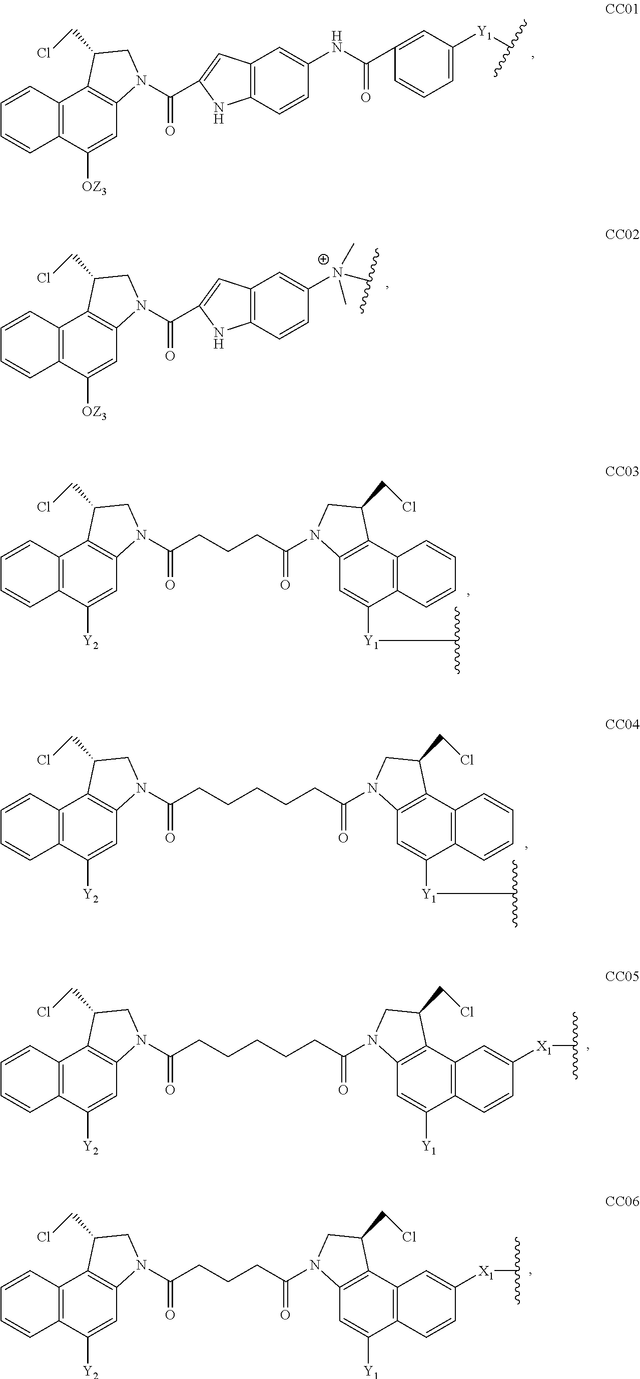 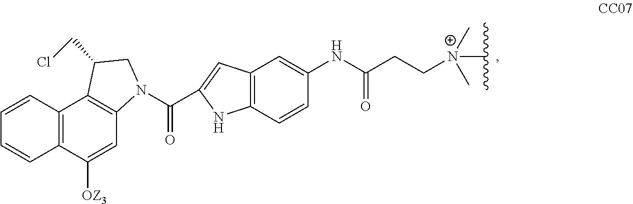
wherein X₁, X₂, Y₁ and Y₂ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₂), C(O)NHNHC(O) or C(O)NR₁ when linked to a connecting site “ [symbol] ”; or OH, NH₂, NHNH₂, NHR₁, SH, C(O)OH, C(O)NH₂, OC(O)NH₂, OC(O)OH, NHC(O)NH₂, NHC(O)SH, OC(O)NH(R₁), N(R₁)C(O)NH(R₂), C(O)NHNHC(O)OH or C(O)NHR₁ when not linked to the connecting site “ [symbol] ”; Z₃ is H, PO(OM₁)(OM₂), SO₃M₁, CH₂PO(OM₁)(OM₂), CH₃N(CH₂CH₂)₂NC(O)—, O(CH₂CH₂)₂NC(O)—, R₁, or glycoside; wherein R₁, R₂, R₃, M₁, M₂, and n are defined the same above;
(L) a Tubulysin and its analogs: 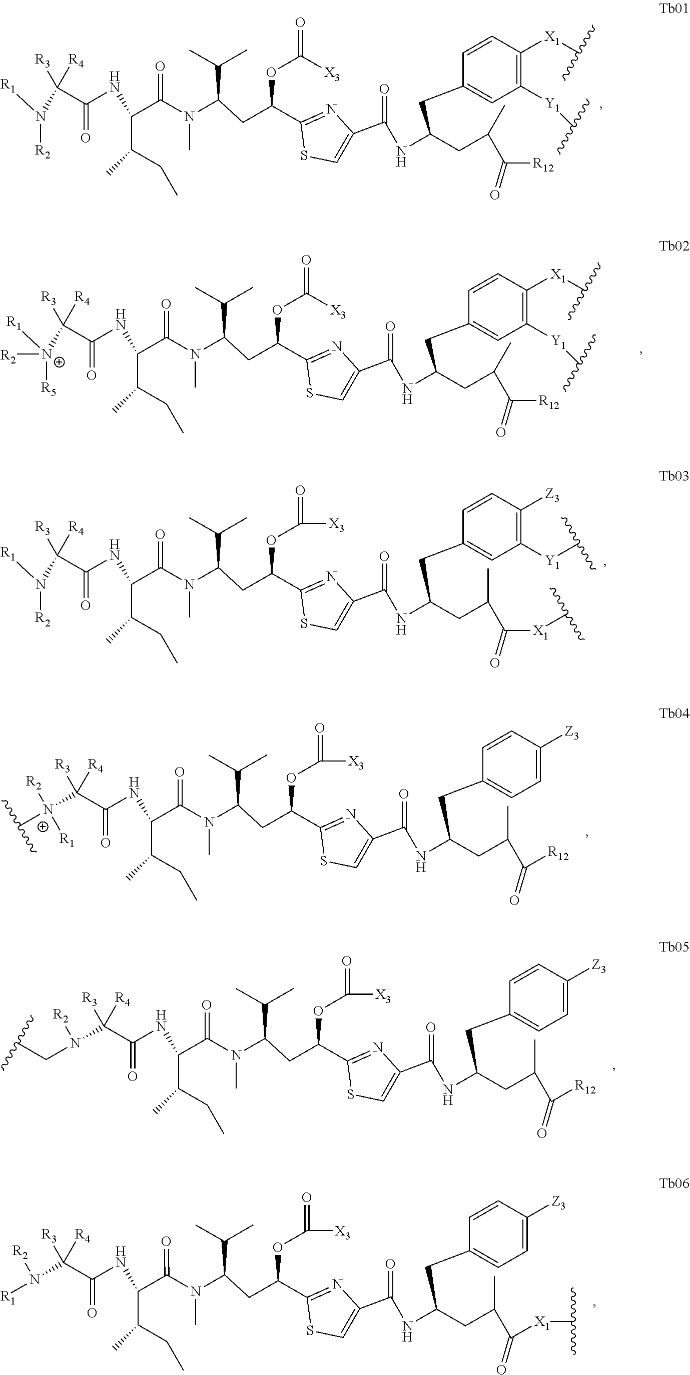 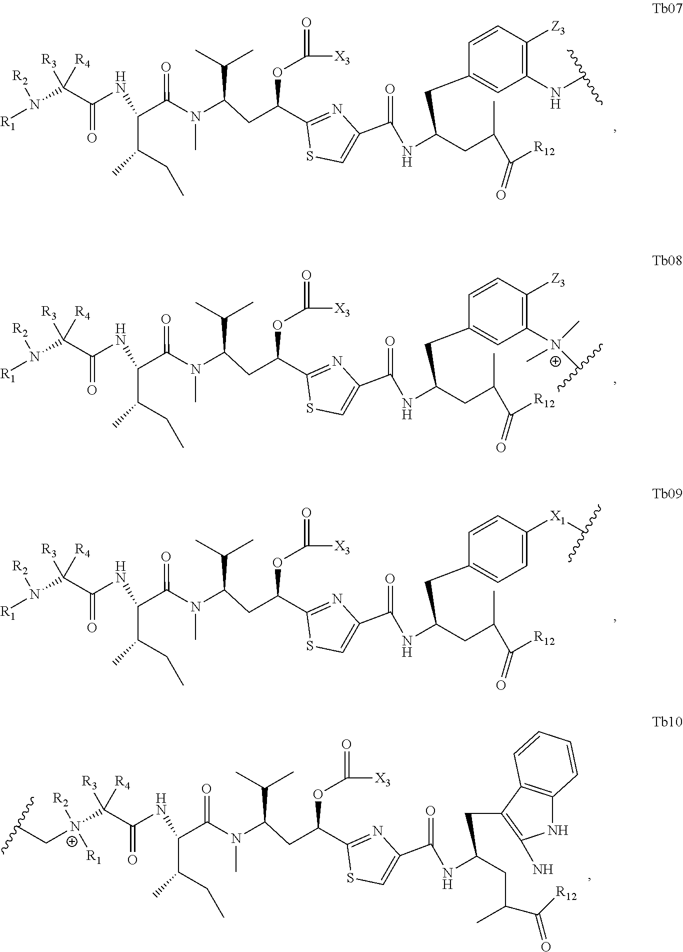
or a derivative thereof with one or more isotopes, or a pharmaceutically acceptable salt, hydrate, or hydrated salt thereof; or a polymorphic crystalline structure thereof; or an optical isomer, racemate, diastereomer or enantiomer thereof; wherein X₁ and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH C(O)NHNHC(O) or C(O)NR₁; mAb is an antibody; R₁₂ is OH, NH₂, NHR₁, NHNH₂, NHNHCOOH, O—R₁—COOH, NH—R₁—COOH, NH-(Aa)ₙCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R¹′, NHOH, NHOR₁, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NHPO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂CH₂OPO₃H₂, OR₁—NHPO₃H₂, NH—R₁—NHPO₃H₂, NH(CH₂CH₂NH)ₚCH₂CH₂NH₂, NH(CH₂CH₂S)ₚCH₂CH₂NH₂, NH(CH₂CH₂NH)ₚCH₂CH₂OH, NH(CH₂CH₂S)ₚCH₂CH₂OH, NH—R₁—NH₂, or NH(CH₂CH₂O)ₚCH₂CH₂NHPO₃H₂, wherein Aa is 1-8 aminoacids; n and mi are independently 1-20; p is 1-5000; R₁, R₁′, R₂, R₃, and R₄ are independently H, C₁-C₈ linear or branched alkyl, amide, or amine; C₂-C₈ aryl, alkenyl, alkynyl, heteroaryl, heteroalkyl, alkylcycloalkyl, ester, ether, heterocycloalkyl, or acyloxylamine; or peptide containing 1-8 aminoacids, or polyethyleneoxy unit having formula (OCH₂CH₂)ₚ or (OCH₂CH(CH₃))ₚ, wherein p is an integer from 1 to about 5000; or two Rs: R₁ and R₂, R₂ and R₃, R₁ and R₃ or R₃ and R₄ can form a 3-8 member cyclic ring of alkyl, aryl, heteroaryl, heteroalkyl, or alkylcycloalkyl group; X₃ is H, CH₃, CH₂CH₃, C₃H₇, or X₁′R₁′, wherein X₁′ is NH, N(CH₃), NHNH, O, or S; R₁′ is H or C₁-C₈ linear or branched alkyl, aryl, heteroaryl, heteroalkyl, alkylcycloalkyl, or acyloxylamine; R₃′ is H or C₁-C₆ linear or branched alkyl; Z₃ is H, COOR₁, NH₂, NHR₁, OR₁, CONHR₁, NHCOR₁, OCOR₁, OP(O)(OM₁)(OM₂), OCH₂OP(O)(OM₁)(OM₂), OSO₃M₁, R₁, O-glycoside (glucoside, galactoside, mannoside, glucuronoside/glucuronide, alloside, fructoside), NH-glycoside, S-glycoside or CH₂-glycoside; M₁ and M₂ are independently H, Na, K, Ca, Mg, NH₄, or NR₁R₂R₃;
(M) an Amatoxin and its analogs: 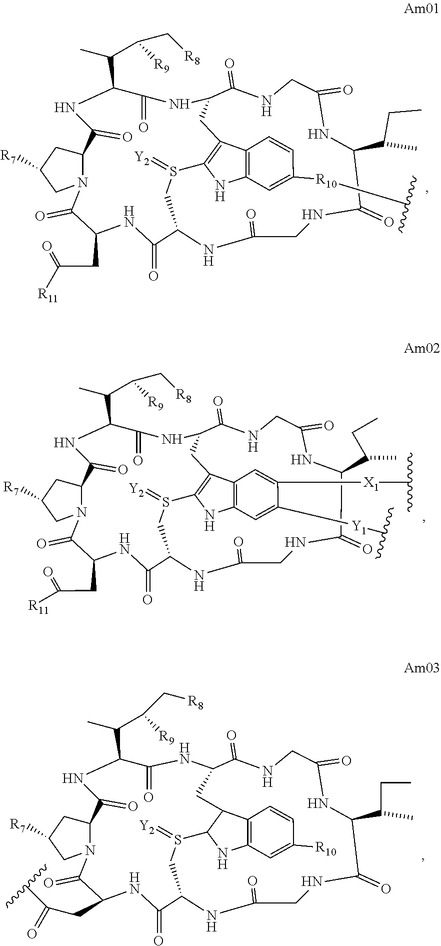
or a derivative thereof with one or more isotopes, or a pharmaceutically acceptable salt, hydrate, or hydrated salt thereof; or a polymorphic crystalline structure thereof; or an optical isomer, racemate, diastereomer or enantiomer thereof; wherein X₁ and Y₁ are independently O, NH, NHNH, NR₅, S, C(O)O, C(O)NH, OC(O)NH, OC(O)O, NHC(O)NH, NHC(O)S, OC(O)N(R₁), N(R₁)C(O)N(R₁), CH, C(O)NHNHC(O) or C(O)NR₁; R₇, R₈, and R₉ are independently H, OH, OR₁, NH₂, NHR₁, C₁-C₆ alkyl, or absent; Y₂ is O, O₂, NR₁, NH, or absent; R₁₀ is CH₂, O, NH, NR₁, NHC(O), NHC(O)NH, NHC(O)O, OC(O)O, C(O), OC(O), OC(O)(NR₁), (NR₁)C(O)(NR₁), C(O)R₁ or absent; R₁₁ is OH, NH₂, NHR₁, NHNH₂, NHNHCOOH, O—R₁—COOH, NH—R₁—COOH, NH-(Aa)ᵣCOOH, O(CH₂CH₂O)ₚCH₂CH₂OH, O(CH₂CH₂O)ₚCH₂CH₂NH₂, NH(CH₂CH₂O)ₚCH₂CH₂NH₂, NR₁R¹′, O(CH₂CH₂O)ₚCH₂CH₂COOH, NH(CH₂CH₂O)ₚCH₂CH₂COOH, NH—Ar—COOH, NH—Ar—NH₂, O(CH₂CH₂O)ₚCH₂CH₂NHSO₃H, NH(CH₂CH₂O)ₚCH₂CH₂NHSO₃H, R₁—NHSO₃H, NH—R₁—NHSO₃H, O(CH₂CH₂O)ₚCH₂CH₂NH—PO₃H₂, NH(CH₂CH₂O)ₚCH₂CH₂NHPO₃H₂, OR₁, R₁—NHPO₃H₂, R₁—OPO₃H₂, O(CH₂CH₂O)ₚCH₂—CH₂OPO₃H₂, OR₁—NHPO₃H₂, NH—R₁—NHPO₃H₂, or NH(CH₂CH₂O)ₚCH₂CH₂NHPO₃H₂, wherein (Aa)ᵣ is 1-8 aminoacids; n and mi are independently 1-20; p is 1-5000; R₁ and Ar, are the same defined in claim 22;
(N) a Protein kinase inhibitor: 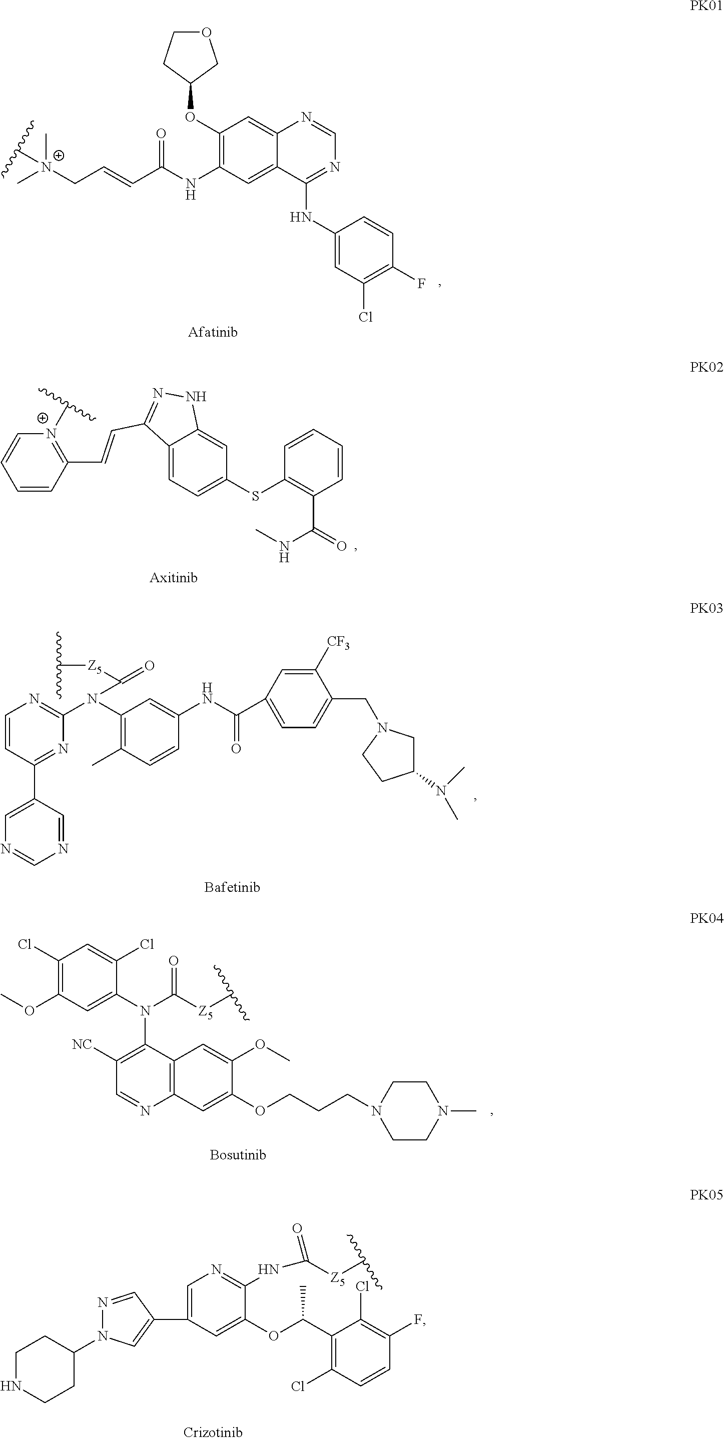 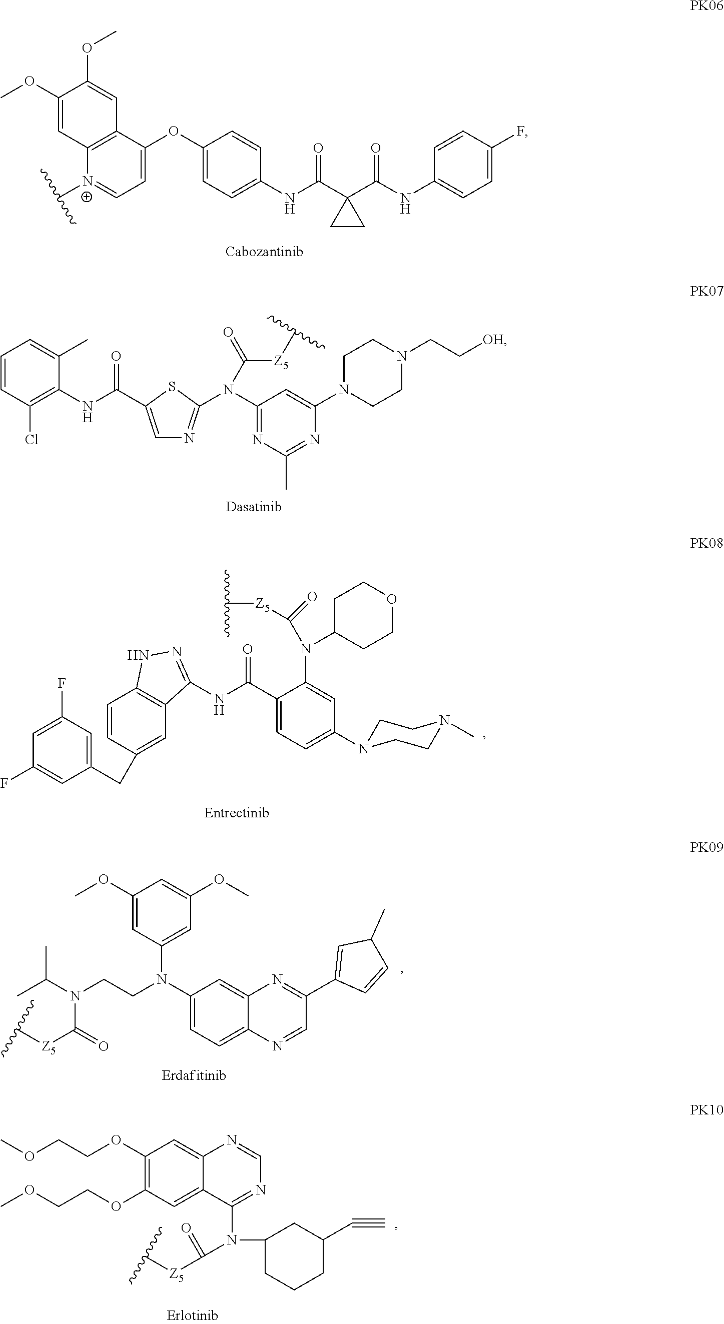 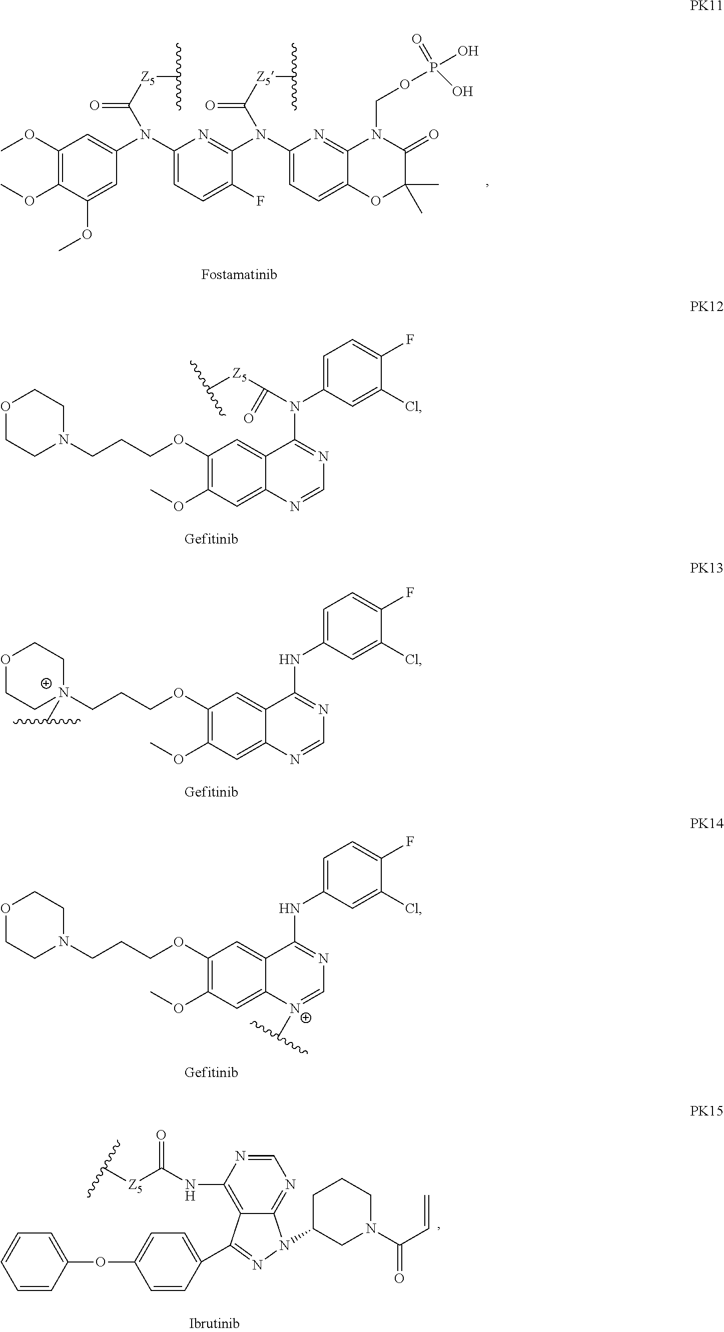 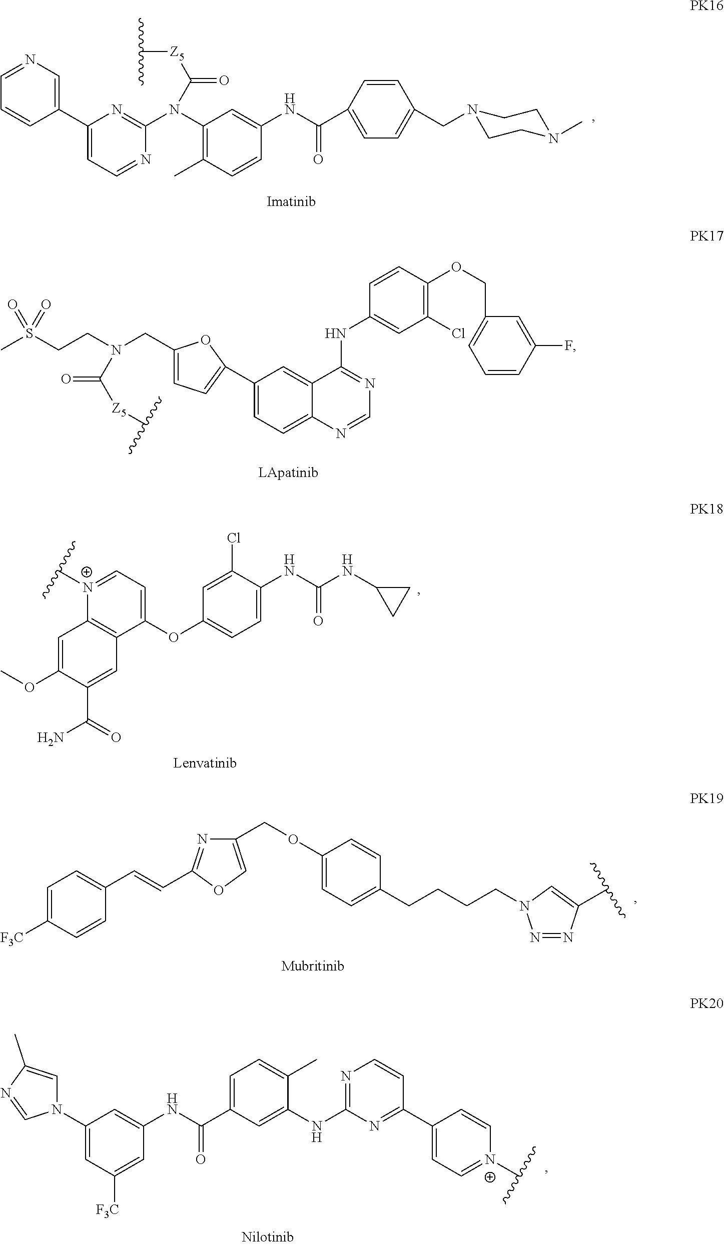 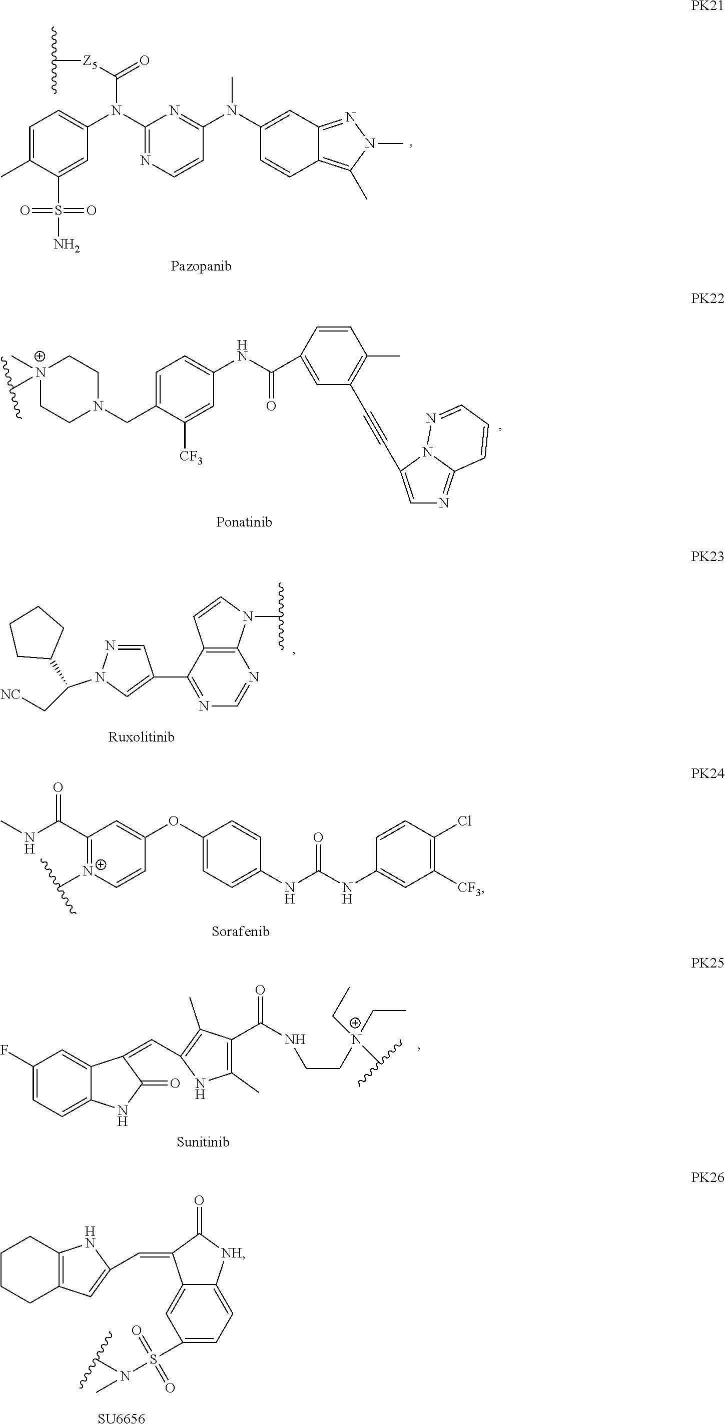 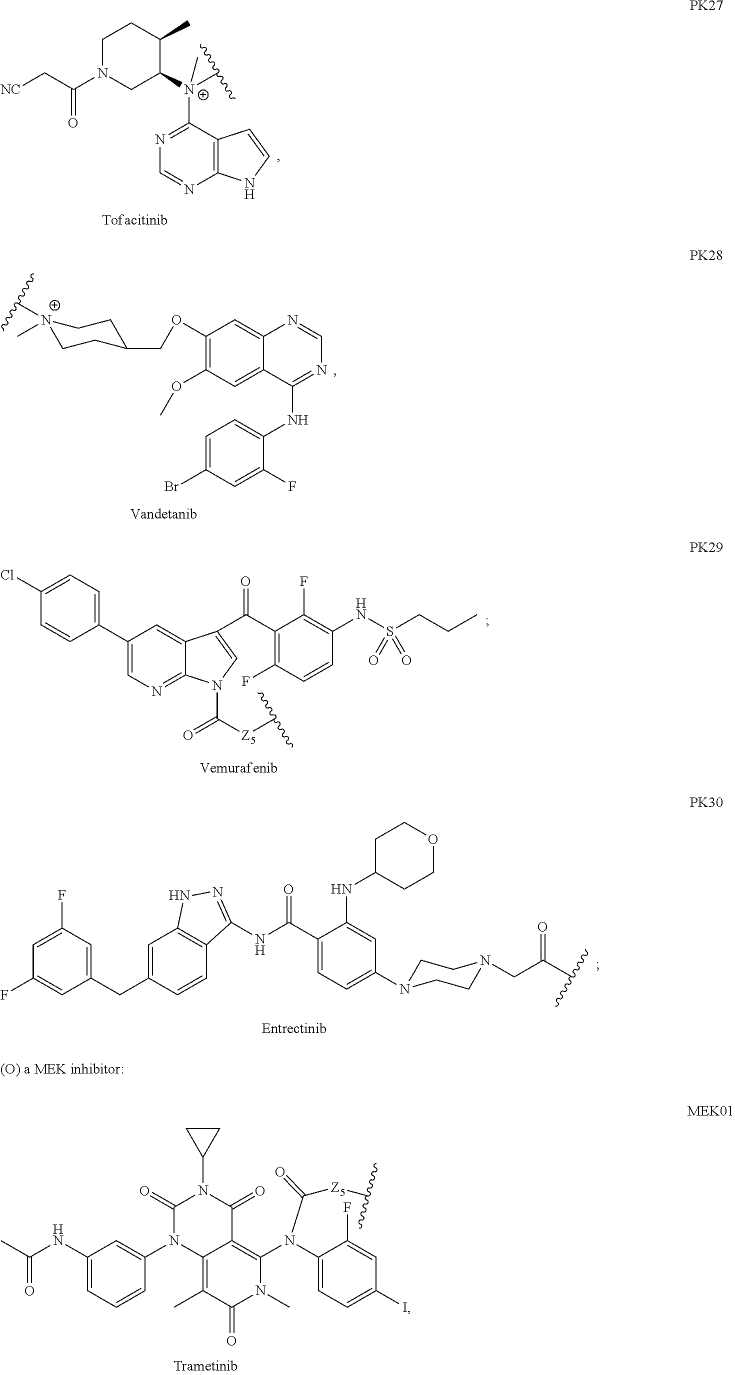 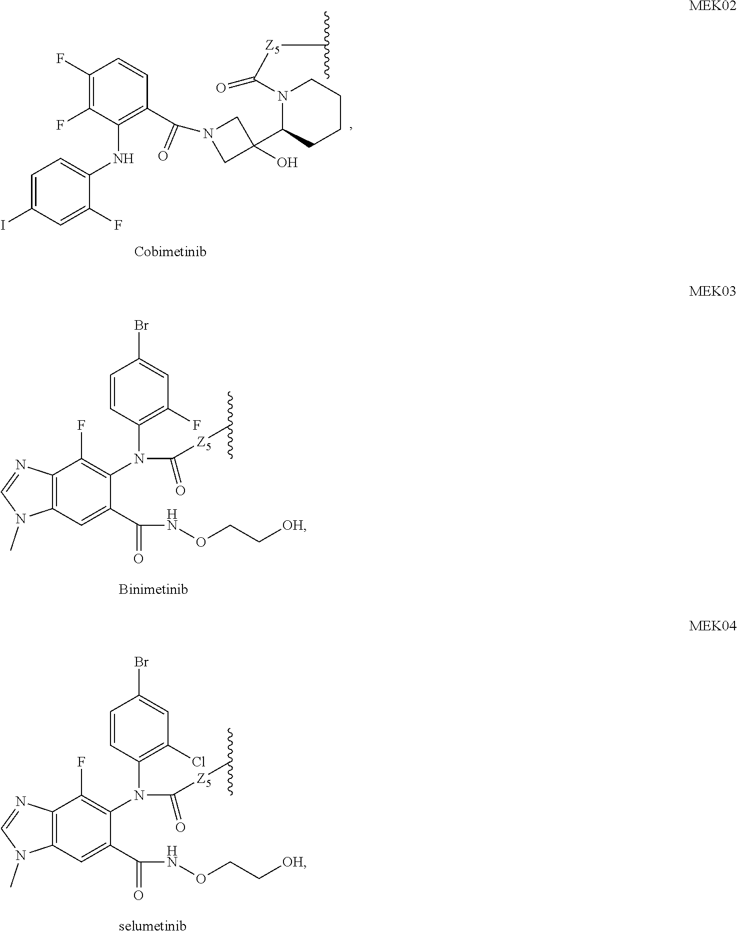
wherein Z₅ is O, NH, NHNH, NR₅, S, C(O)O, C(O)NH, OC(O)NH, OC(O)O, NHC(O)O, NHC(O)NH, NHC(O)S, OC(O)N(R₁), N(R₁)C(O)N(R₂), C(O)NHNHC(O) or C(O)NR₁;
(P) a proteinase inhibitor: 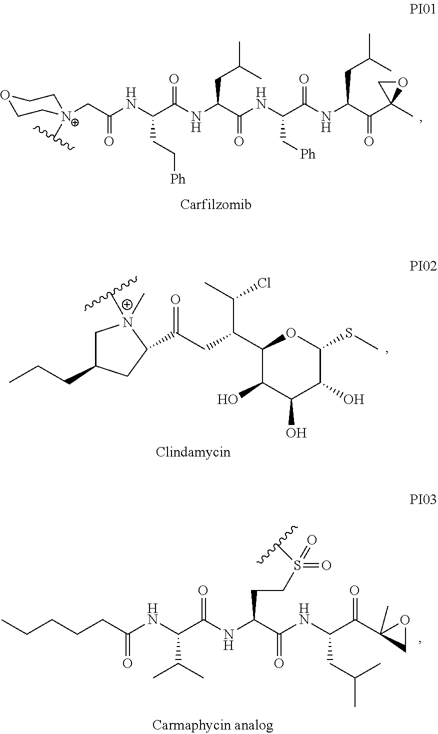
(Q) an immunotoxin that is a macromolecular drug which is a cytotoxic protein derived from a bacterial or plant protein, and is Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins, proaerolysin, or topsalysin, and the immunotoxin conjugates via the side-chain linker through its amino acid having an amine, thiol or carboxylic acid group.

28. The compound according to claim 22, wherein W, L₁, L₂, V₁, and V₂ independently contain one or more linker components of the following structures: 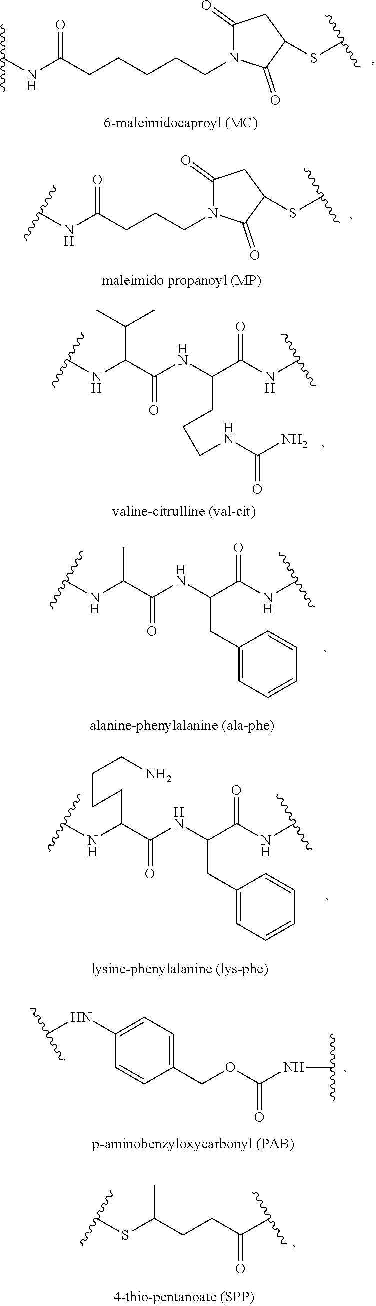 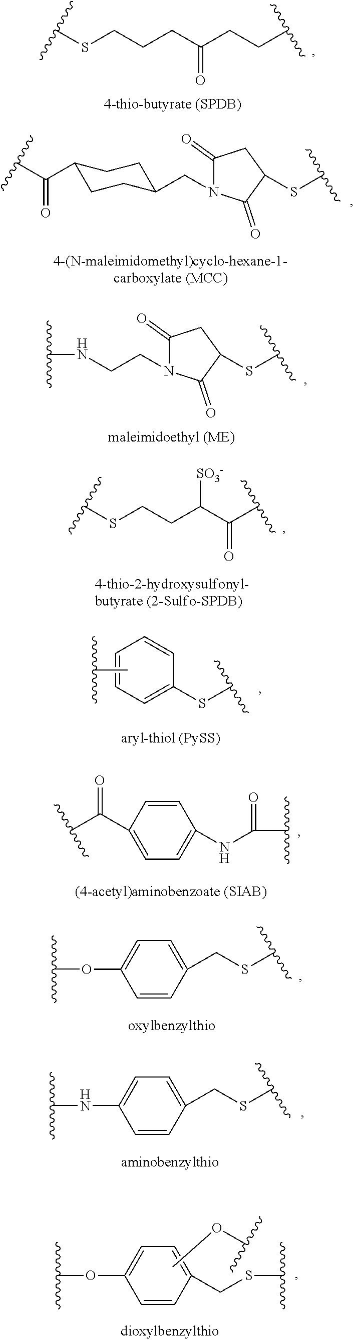 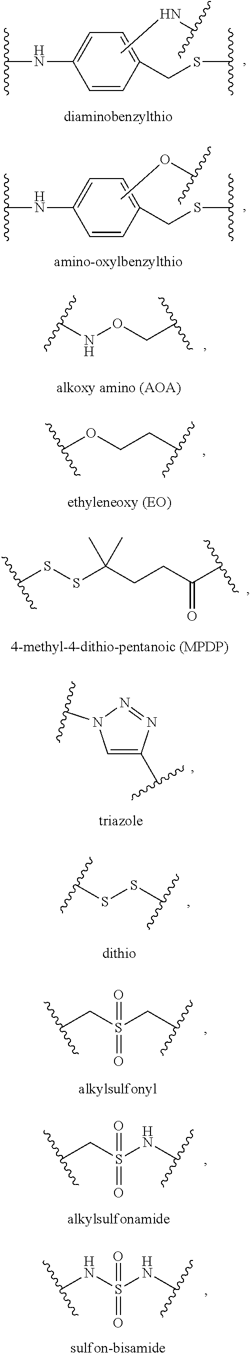 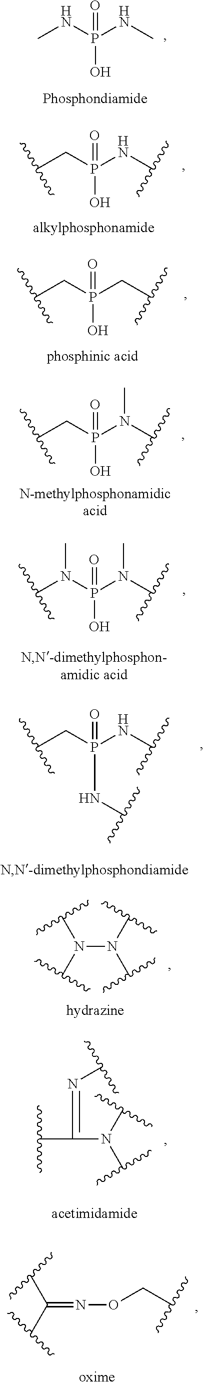 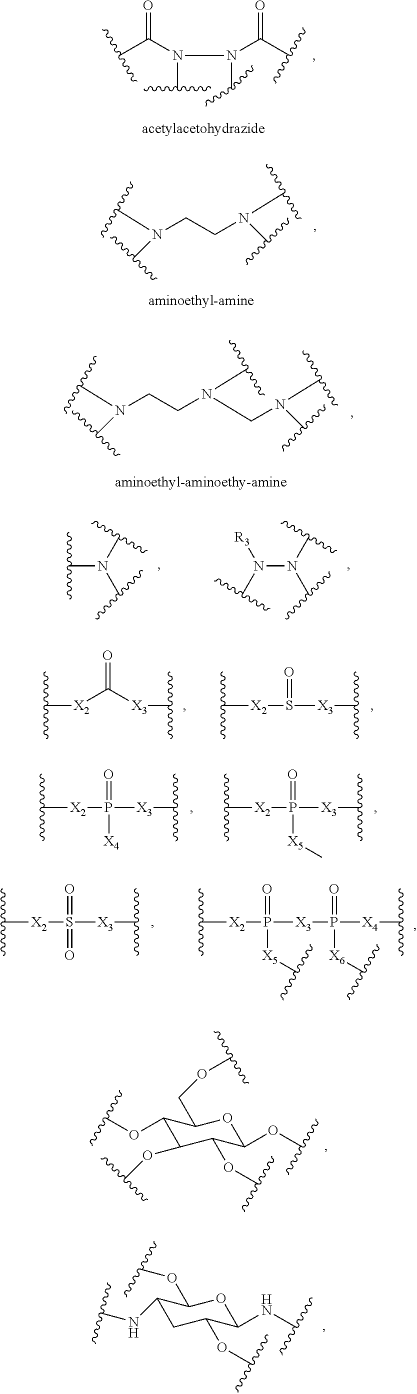 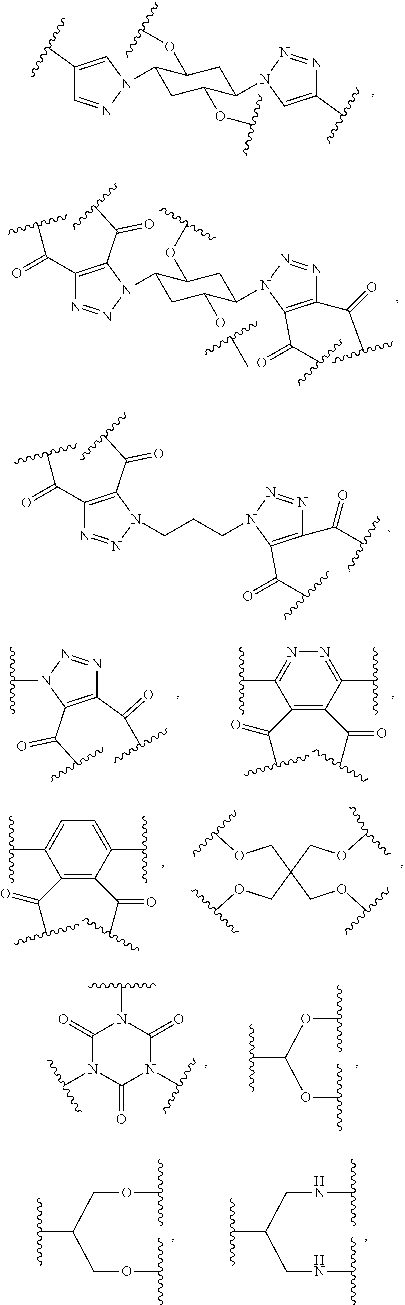 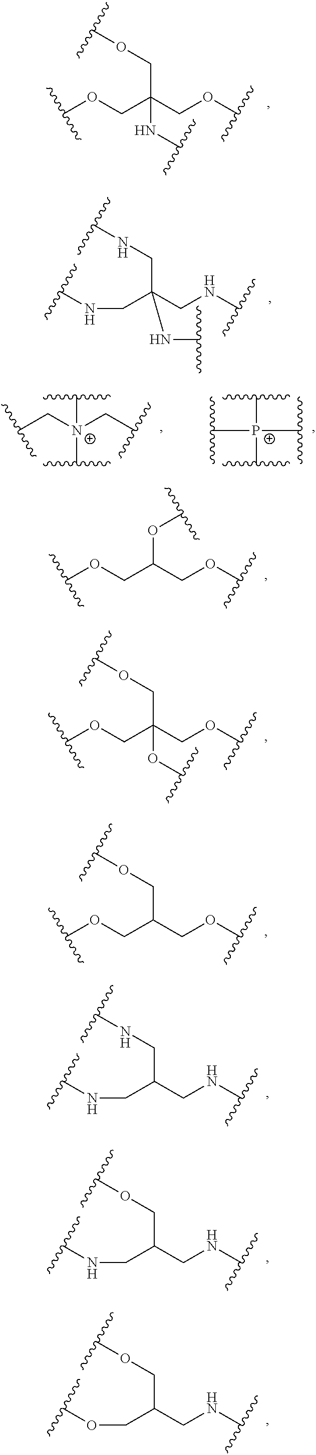
or L- or D-, natural or unnatural peptides containing 1-20 the same or different amino acids; wherein [symbol] is a site of linkage; X₂, X₃, X₄, X₅, and X₆, are independently NH; NHNH; N(R₁₂); N(R₁₂)N(R_{12′}); O; S; C₁-C₆ alkyl; C₂-C₆ heteroalkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, heteroaryl; CH₂OR₁₂, CH₂SR₁₂, CH₂NHR₁₂, or 1-8 amino acids; wherein R₁₂ and R_{12′} are independently H; C₁-C₈ alkyl; C₂-C₈ hetero-alkyl, alkylcycloalkyl, or heterocycloalkyl; C₃-C₈ aryl, Ar-alkyl, heterocyclic, carbocyclic, cycloalkyl, heteroalkylcycloalkyl, alkylcarbonyl, or heteroaryl; or C₁-C₈ ester, ether, or amide; or polyethyleneoxy unit of formula (OCH₂CH₂)ₚ or (OCH₂CH(CH₃))ₚ, wherein p is an integer from 0 to about 1000, or a combination of two or more of the above groups.

29. The compound according to claim 22, wherein W, L₁, L₂, V₁, and V₂ independently contain:
(A): a self-immolative component, peptidic unit, a hydrazone bond, a disulfide, an ester, an oxime, an amide, or a thioether bond; the self-immolative unit includes an aromatic compound that is electronically similar to para-aminobenzyl-carbamoyl (PAB) groups, 2-aminoimidazol-5-methanol derivative, heterocyclic PAB analog, beta-glucuronide, or ortho or para-aminobenzylacetal; or one of the following structures: 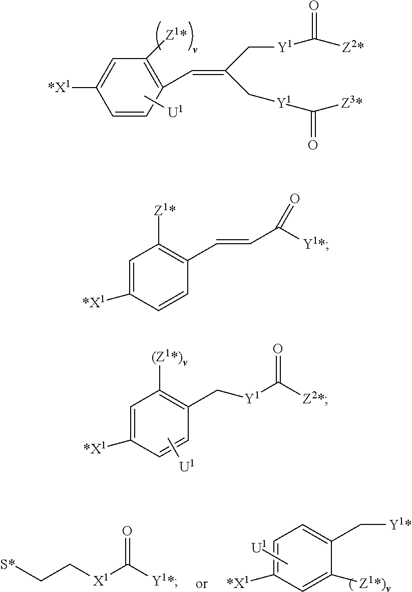
wherein a (*) atom is a point of attachment of another component; X¹, Y¹, Z² and Z³ are independently NH, O, or S; Z¹ is independently H, NHR₁, OR₁, SR₁, or COX₁R₁, wherein X₁ and R₁ are defined above; v is 0 or 1; U¹ is independently H, OH, C₁-C₆ alkyl, (OCH₂CH₂)ₙ, F, Cl, Br, I, OR₅, SR₅, NR₅R₅′, N═NR₅, N═R₅, NR₅R₅′, NO₂, SOR₅R₅′, SO₂R₅, SO₃R₅, OSO₃R₅, PR₅R₅′, POR₅R₅′, PO₂R₅R₅′, OPO(OR₅)(OR₅′), or OCH₂PO(OR₅(OR₅′), wherein R₅ and R₅′ are independently H, C₁-C₈ alkyl; C₂-C₈ alkenyl, alkynyl, heteroalkyl, or amino acid; C₃-C₈ aryl, heterocyclic, carbocyclic, cycloalkyl, heterocycloalkyl, heteroaralkyl, alkylcarbonyl, or glycoside; or a pharmaceutical cation salt thereof;
(B): a non-self-immolative linker component containing one of the following structures: 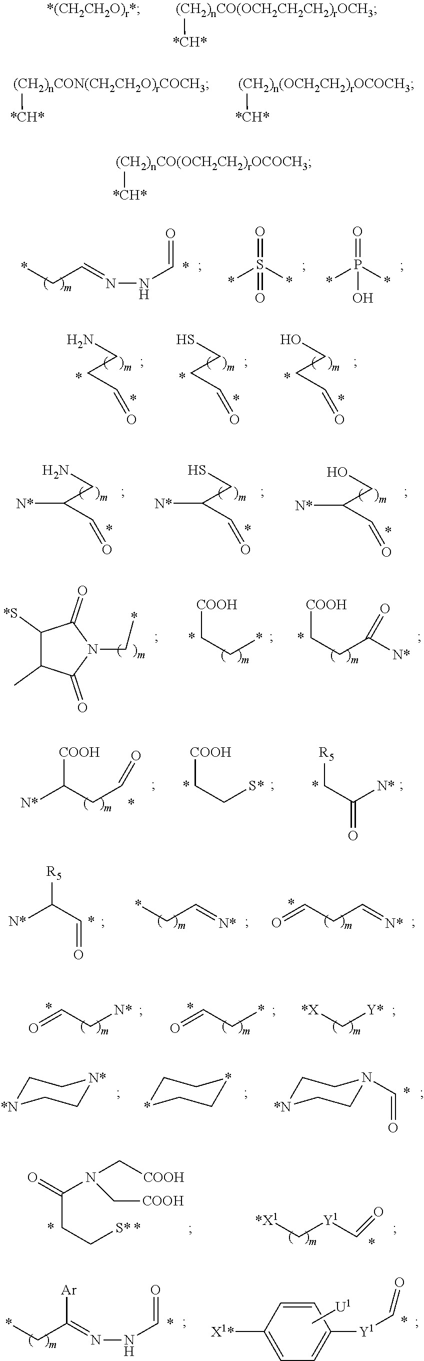 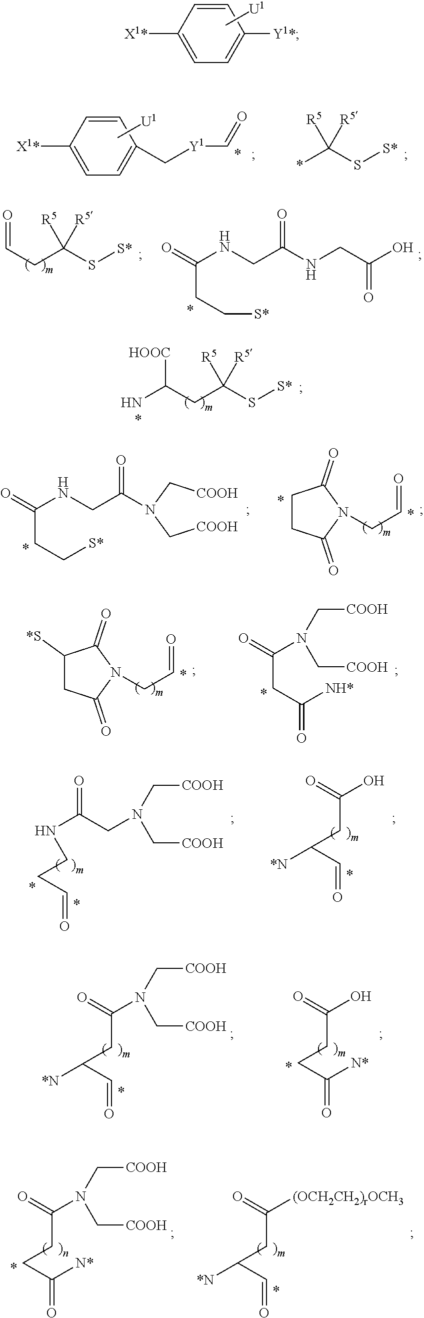 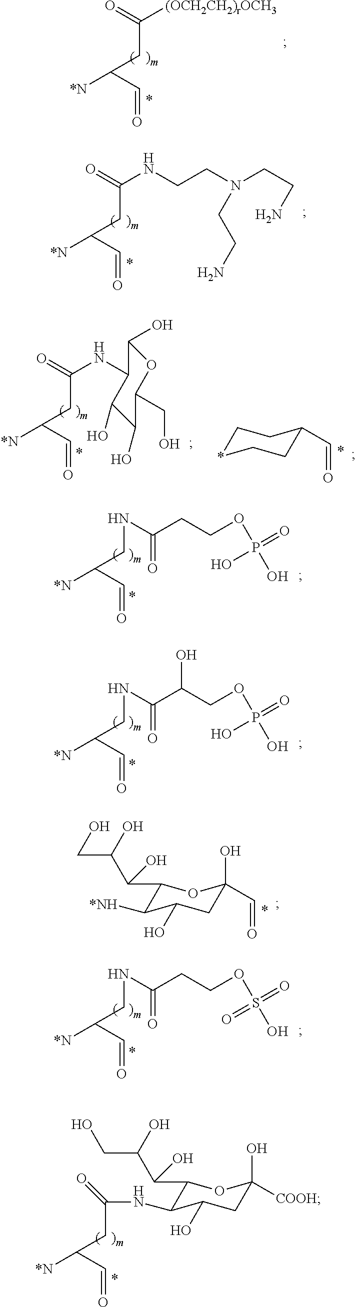 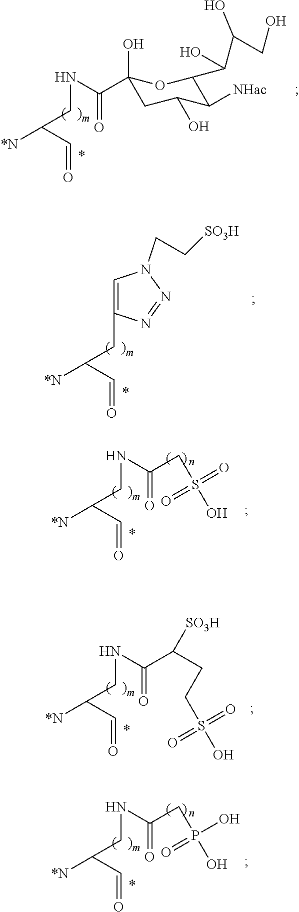
wherein a (*) atom is a point of attachment of additional spacer or releasable linker, the cytotoxic agent, and/or the binding molecule; X¹, Y¹, U¹, R⁵, R^{5′} are defined as above; r is 0-100; m and n are 0-20 independently;
(C): a releasable component that contains at least one bond that can be broken under physiological conditions: a pH-labile, acid-labile, base-labile, oxidatively labile, metabolically labile, biochemically labile or enzyme-labile bond, and has one of the following structures:
—(CR₁₅R₁₆)ₘ(Aa)r(CR₁₇R₁₈)ₙ(OCH₂CH₂)ₜ—, —(CR₁₅R₁₆)ₘ(CR₁₇R₁₈)ₙ(Aa)ᵣ(OCH₂CH₂)ₜ—, - (Aa)ᵣ-(CR₁₅R₁₆)ₘ(CR₁₇R₁₈)ₙ(OCH₂CH₂)ₜ—, —(CR₁₅R₁₆)ₘ(CR₁₇R₁₈)ₙ(OCH₂CH₂)ᵣ(Aa)ₜ-, —(CR₁₅R₁₆)ₘ(CR₁₇═CR₁₈)(CR₁₉R₂₀)ₙ(Aa)ₜ(OCH₂CH₂)ᵣ—, —(CR₁₅R₁₆)ₘ(NR₁₁CO)(Aa)ₜ(CR₁₉R₂₀)ₙ—(OCH₂CH₂)ᵣ—, —(CR₁₅R₁₆)ₘ(Aa)ₜ(NR₂₁CO)(CR₁₉R₂₀)ₙ(OCH₂CH₂)ᵣ—, —(CR₁₅R₁₆)ₘ(OCO)(Aa)ₜ-(CR₁₉R₂₀)ₙ(OCH₂CH₂)ᵣ—, —(CR₁₅R₁₆)ₘ(OCNR₁₇)(Aa)ₜ(CR₁₉R₂₀)ₙ(OCH₂CH₂)ᵣ—, —(CR₁₅R₁₆)ₘ—(CO)(Aa)ₜ-(CR₁₉R₂₀)ₙ(OCH₂CH₂)ᵣ—, —(CR₁₅R₁₆)ₘ(NR₂₁CO)(Aa)ₜ(CR₁₉R₂₀)ₙ(OCH₂CH₂)ᵣ—, —(CR₁₅R₁₆)ₘ—(OCO)(Aa)ₜ(CR₁₉R₂₀)ₙ—(OCH₂CH₂)ᵣ—, —(CR₁₅R₁₆)ₘ(OCNR₁₇)(Aa)ₜ(CR₁₉R₂₀)ₙ—(OCH₂CH₂)ᵣ—, —(CR₁₅R₁₆)ₘ(CO)(Aa)ₜ(CR₁₉R₂₀)ₙ—(OCH₂CH₂)ᵣ—, —(CR₁₅R₁₆)ₘ-phenyl-CO(Aa)ₜ-(CR₁₇R₁₈)ₙ—, —(CR₁₅R₁₆)ₘ-furyl-CO(Aa)ₜ(CR₁₇R₁₈)ₙ—, —(CR₁₅R₁₆)ₘ-oxazolyl-CO(Aa)ₜ(CR₁₇R₁₈)ₙ—, —(CR₁₅R₁₆)ₘ-thiazolyl-CO(Aa)ₜ(CCR₁₇R₁₈)ₙ—, —(CR₁₅R₁₆)ₜ-thienyl-CO(CR₁₇R₁₈)ₙ—, —(CR₁₅R₁₆)ₜ-imidazolyl-CO—(CR₁₇R₁₈)ₙ—, —(CR₁₅R₁₆)ₜ-morpholino-CO(Aa)ₜ-(CR₁₇R₁₈)ₙ—, —(CR₁₅R₁₆)ₜ-piperazino-CO(Aa)ₜ(CR₁₇R₁₈)ₙ—, —(CR₁₅R₁₆)ₜ—N-methylpiperazin-CO(Aa)ₜ(CR₁₇R₁₈)ₙ—, —(CR₁₅R₁₆)ₘ-(Aa)ₜphenyl-, —(CR₁₅R₁₆)ₘ-(Aa)ₜfuryl-, —(CR₁₅R₁₆)ₘ-oxazolyl(Aa)ₜ-, —(CR₁₅R₁₆)ₘ-thiazolyl(Aa)ₜ-, —(CR₁₅R₁₆)ₘ-thienyl-(Aa)ₜ-, —(CR₁₅R₁₆)ₘ-imidazolyl(Aa)ₜ-, —(CR₁₅R₁₆)ₘ-morpholino-(Aa)ₜ-, —(CR₁₅R₁₆)ₘ-piperazino-(Aa)ₜ-, —(CR₁₅R₁₆)ₘ—N-methylpiperazino-(Aa)ₜ-,
—K(CR₁₅R₁₆)ₘ(Aa)r(CR₁₇R₁₈)ₙ(OCH₂CH₂)ₜ—, —K(CR₁₅R₁₆)ₘ(CR₁₇R₁₈)ₙ(Aa)ᵣ(OCH₂CH₂)ₜ—, —K(Aa)ᵣ-(CR₁₅R₁₆)ₘ(CR₁₇R₁₈)ₙ(OCH₂CH₂)ₜ—, —K(CR₁₅R₁₆)ₘ(CR₁₇R₁₈)ₙ(OCH₂CH₂)ᵣ(Aa)ₜ-, —K(CR₁₅R₁₆)ₘ—(CR₁₇═CR₁₈)(CR₁₉R₂₀)ₙ(Aa)ₜ(OCH₂CH₂)ᵣ, —K(CR₁₅R₁₆)ₘ(NR₁₁CO)(Aa)ₜ-(CR₁₉R₂₀)ₙ(OCH₂CH₂)ᵣ—, —K(CR₁₅R₁₆)ₘ(Aa)ₜ(NR₂₁CO)(CR₁₉R₂₀)ₙ(OCH₂CH₂)ᵣ—, —K(CR₁₅R₁₆)ₘ(OCO)(Aa)ₜ(CR₁₉R₂₀)ₙ—(OCH₂CH₂)ᵣ—, —K(CR₁₅R₁₆)ₘ(OCNR₁₇)(Aa)ₜ(CR₁₉R₂₀)ₙ(OCH₂CH₂)ᵣ—, —K(CR₁₅R₁₆)ₘ(CO)(Aa)ₜ-(CR₁₉R₂₀)ₙ(OCH₂CH₂)ᵣ—, —K(CR₁₅R₁₆)ₘ(NR₂₁CO)(Aa)ₜ(CR₁₉R₂₀)ₙ—(OCH₂CH₂)ᵣ—, —K(CR₁₅R₁₆)ₘ—(OCO)(Aa)ₜ(CR₁₉R₂₀)ₙ(OCH₂CH₂)ᵣ—, —K(CR₁₅R₁₆)ₘ(OCNR₁₇)(Aa)ₜ-(CR₁₉R₂₀)ₙ(OCH₂CH₂)ᵣ—, —K—(CR₁₅R₁₆)ₘ(CO)(Aa)ₜ(CR₁₉R₂₀)ₙ(OCH₂CH₂)ᵣ—, —K(CR₁₅R₁₆)ₘ-phenyl-CO(Aa)ₜ(CR₁₇R₁₈)ₙ—, —K—(CR₁₅R₁₆)ₘ-furyl-CO(Aa)ₜ(CR₁₇R₁₈)ₙ—, —K(CR₁₅R₁₆)ₘ-oxazolyl-CO(Aa)ₜ(CR₁₇R₁₈)ₙ—, —K(CR₁₅R₁₆)ₘ-thiazolyl-CO(Aa)ₜ-(CR₁₇R₁₈)ₙ—, —K(CR₁₅R₁₆)ₜ-thienyl-CO(CR₁₇R¹⁸)ₙ—, —K(CR₁₅R₁₆)ₜimidazolyl-CO—(CR₁₇R₁₈)ₙ—, —K(CR₅R₆)ₜmorpholino-CO(Aa)ₜ-(CR₁₇R₁₈)ₙ—, —K(CR₁₅R₁₆)ₜ-piperazino-CO(Aa)ₜ-(CR₁₇R₁₈)ₙ—, —K(CR₁₅SR₁₆)ₜ—N-methylpiperazin-CO(Aa)ₜ(CR₁₇R₁₈)ₙ—, —K(CR₁₅R₁₆)ₘ-(Aa)ₜphenyl, —K—(CR₁₅R₁₆)ₘ-(Aa)ₜfuryl-, —K(CR₁₅R₁₆)ₘ-oxazolyl-(Aa)ₜ-, —K(CR₁₅R₁₆)ₘ-thiazolyl(Aa)ₜ-, —K(CR₁₅R₁₆)ₘ-thienyl-(Aa)ₜ-, —K(CR₁₅R₁₆)ₘ-imidazolyl(Aa)ₜ-, —K(CR₁₅R₁₆)ₘ-morpholino(Aa)ₜ-, —K(CR₁₅R₁₆)ₘ-piperazino(Aa)ₜG, —K(CR₁₅R₁₆)ₘ—N-methyl-piperazino(Aa)ₜ-; wherein m, Aa, m, n, R₁₃, R₁₄, and R₁₅ are described above; t and r are 0-100 independently; R₁₆, R₁₇, R₁₈, R₁₉, and R₂₀ are independently H; halide; C₁-C₈ of alkyl or heteroalkkyl, C₂-C₈ aryl, alkenyl, alkynyl, ether, ester, amine or amide, C₃-C₈ aryl, which optionally substituted by one or more halide, CN, NR₁₂R_{12′}, CF₃, OR₁₂, Aryl, heterocycle, S(O)R₁₂, SO₂R₁₂, —CO₂H, —SO₃H, —OR₁₂, —CO₂R₁₂, —CONR₁₂, —PO₂R₁₂R₁₃, —PO₃H or P(O)R₁₂R_{12′}R₁₃; K is NR₁₂, —SS—, —C(═O)—, —C(═O)NH—, —C(═O)O—, —C═NH—O—, —C═N—NH—, —C(═O)NH—NH—, O, S, Se, B, Het (heterocyclic or heteroaromatic ring having C₃-C₁₂); or peptide containing the same or different 1-20 amino acids.

30. The conjugate compound according to claim 22 having one of the following structures of a001 to a198, 49 (C-30), 50 (C-40), 51 (C-48), 78, 125, 141, 149, 163, 171, 174 (C-173), 179, 187, C-238, C-247, C-255, C-271, C-279, C-312, C-313, 132 (C-131), 135 (C-134), C-321, and C-322 illustrated below: 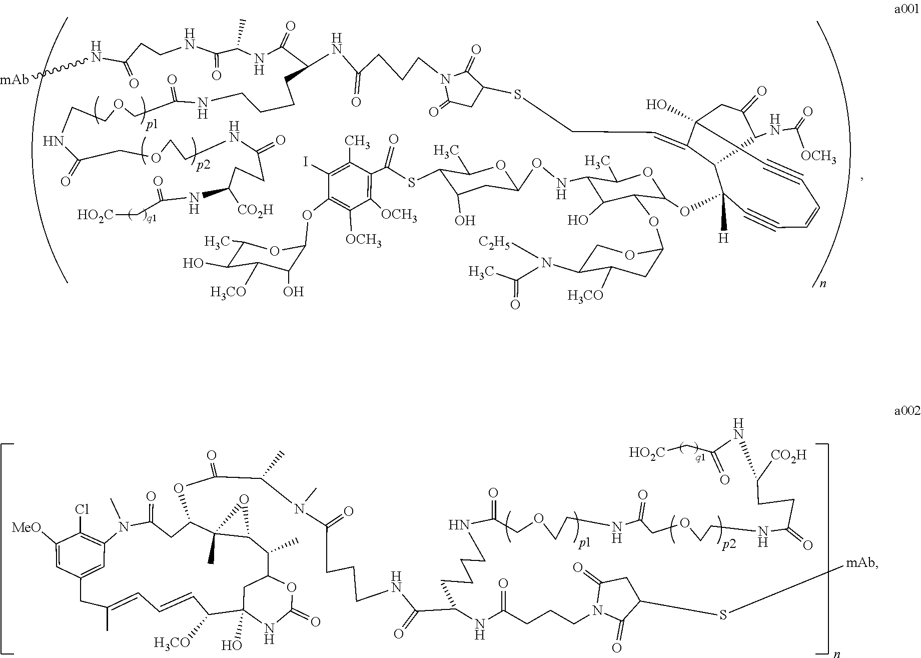 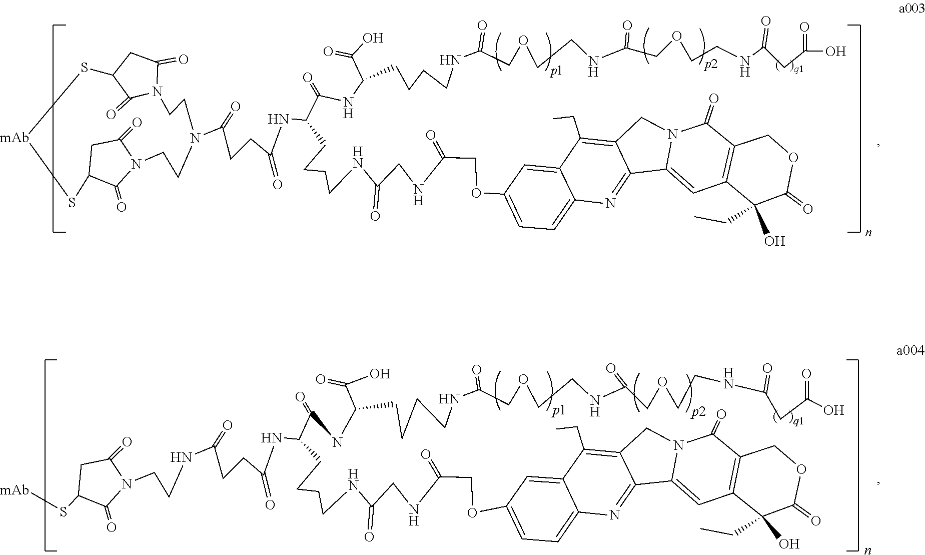 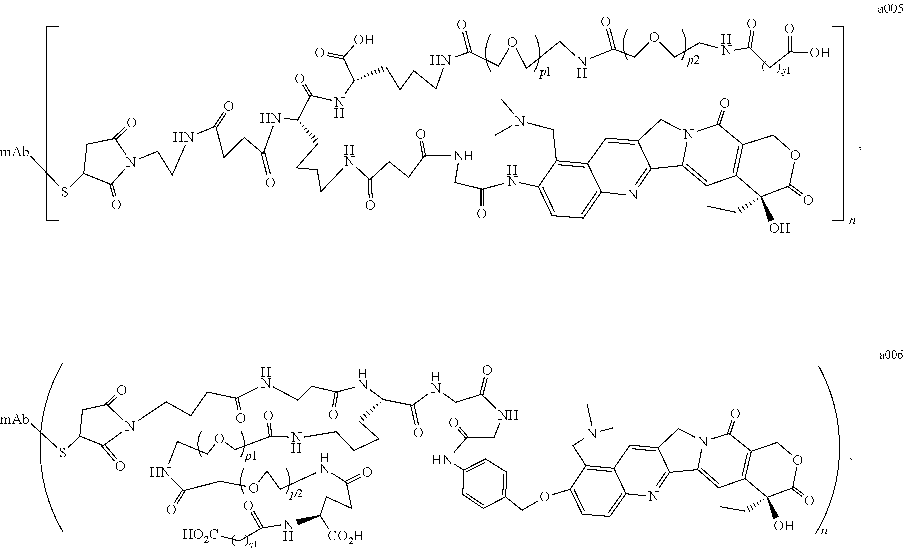 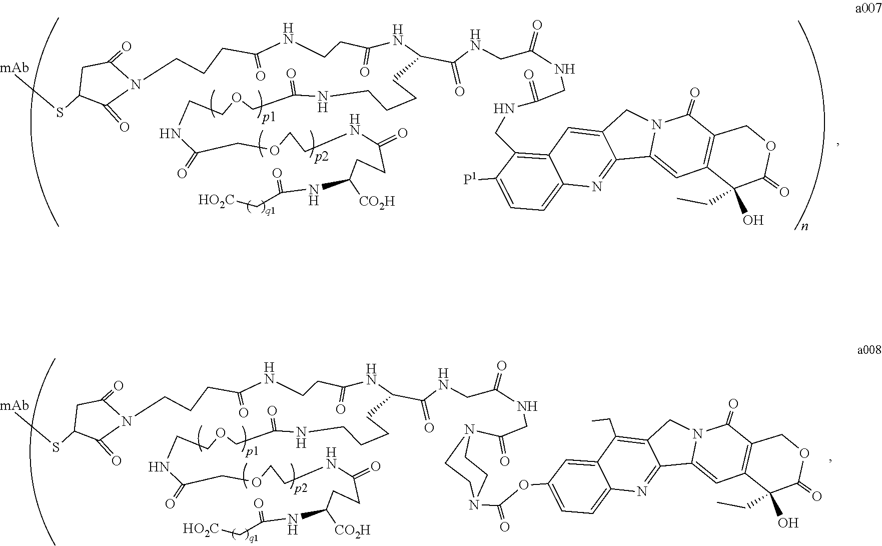 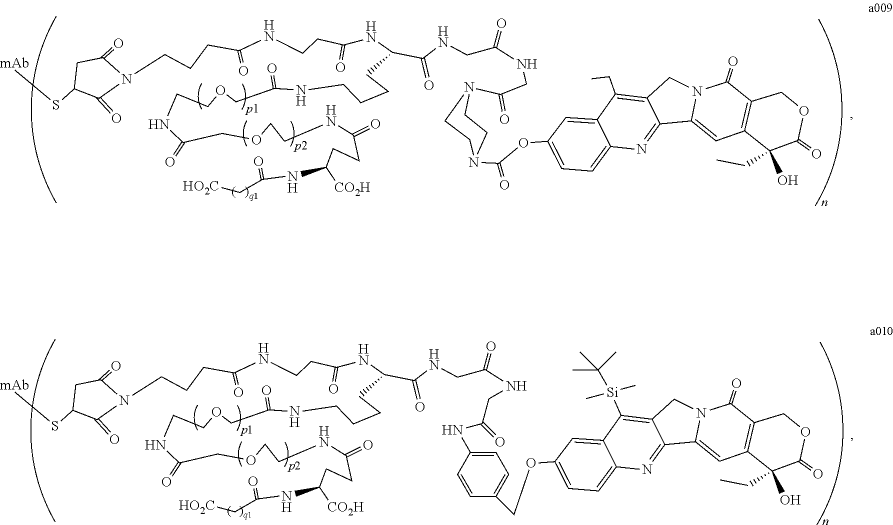 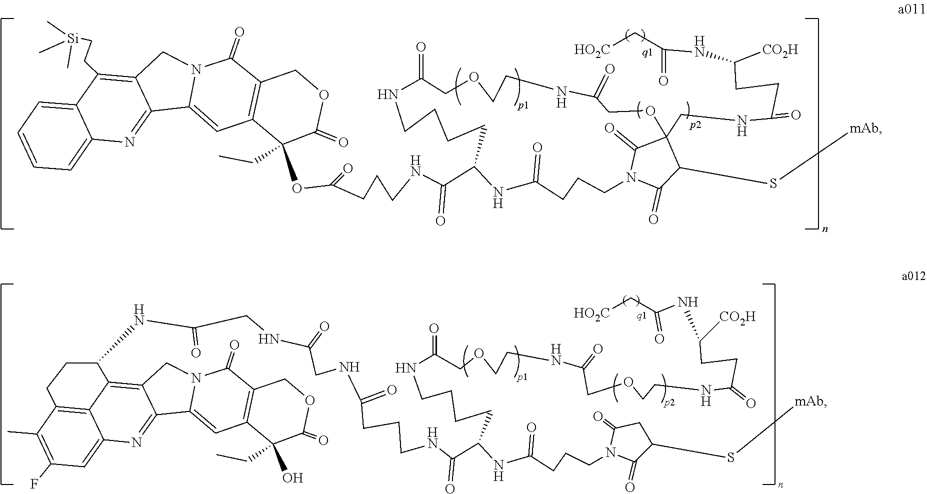 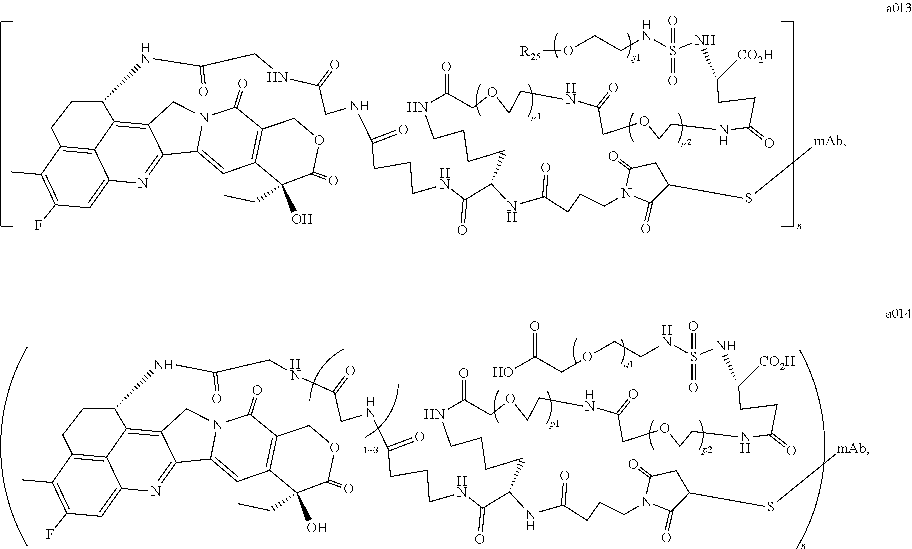 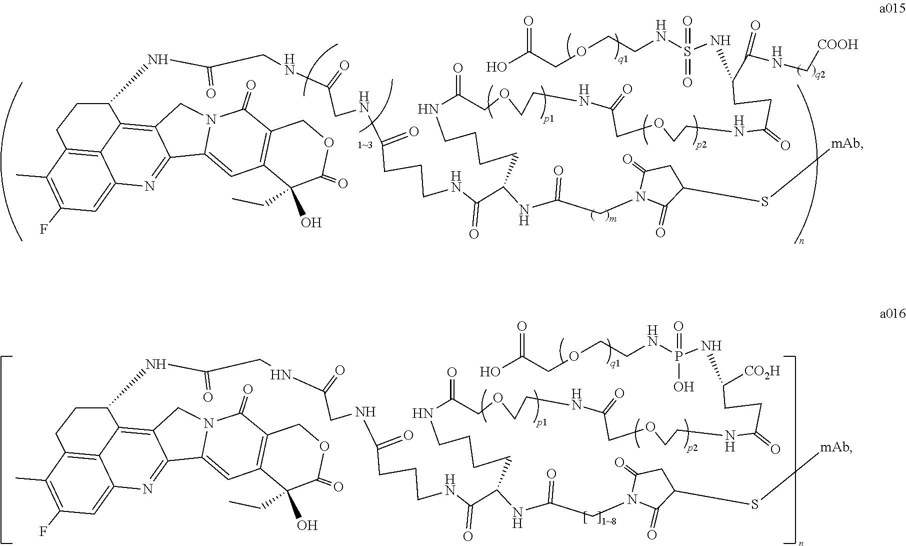 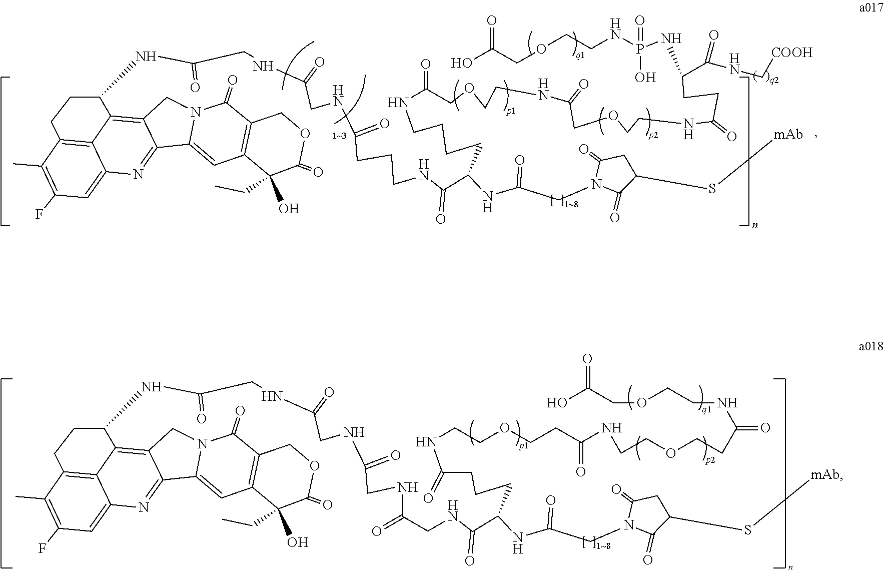 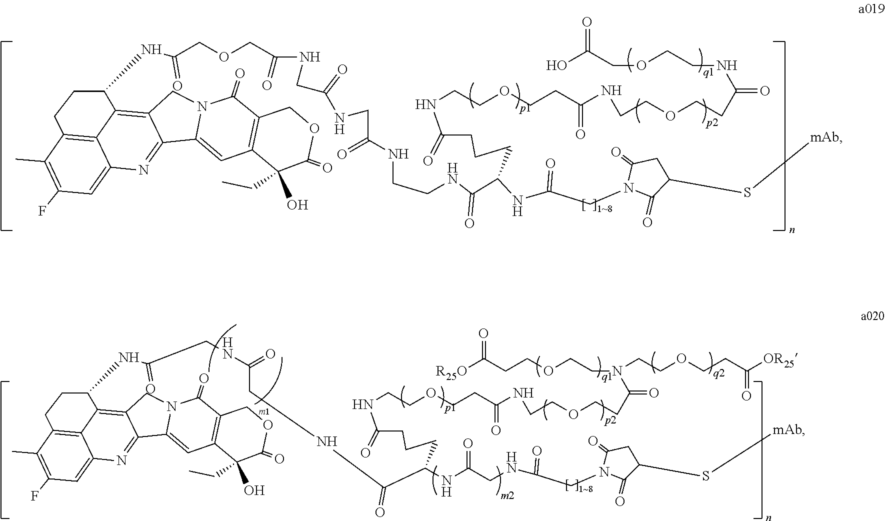 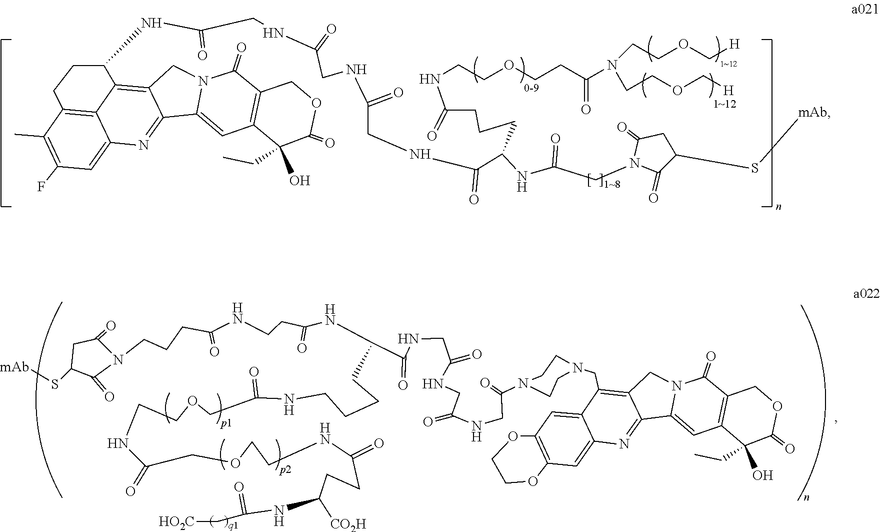 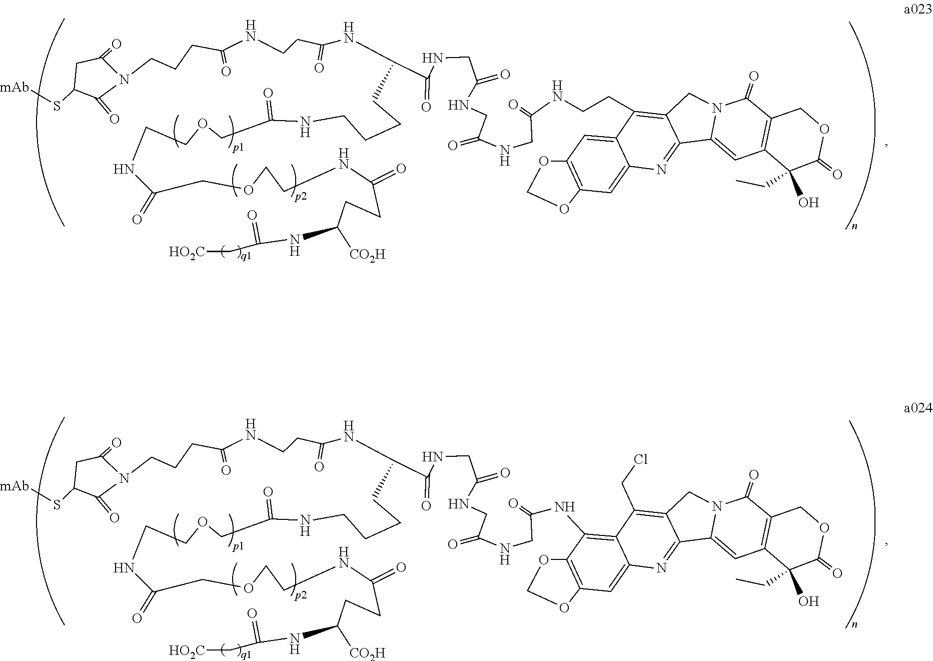 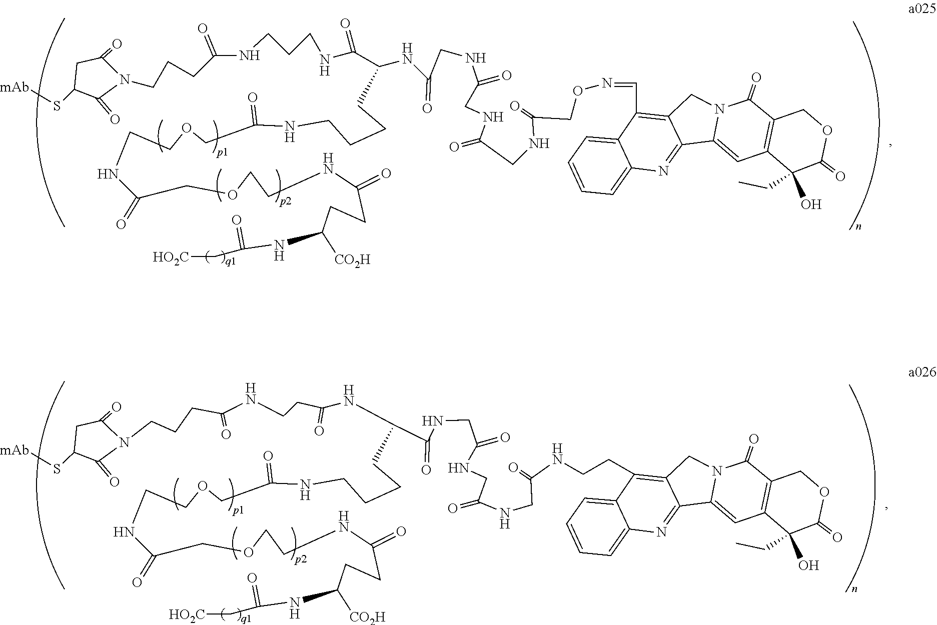 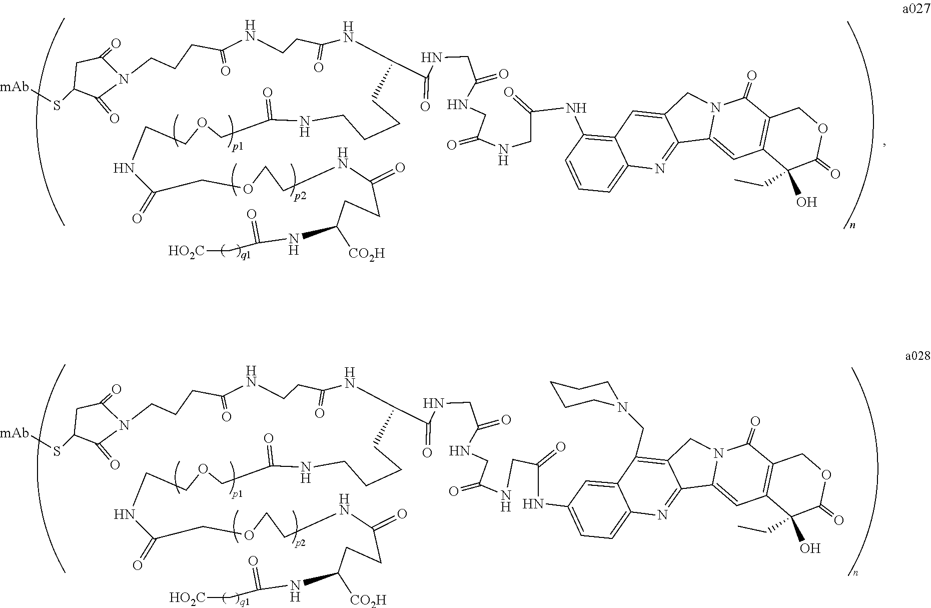 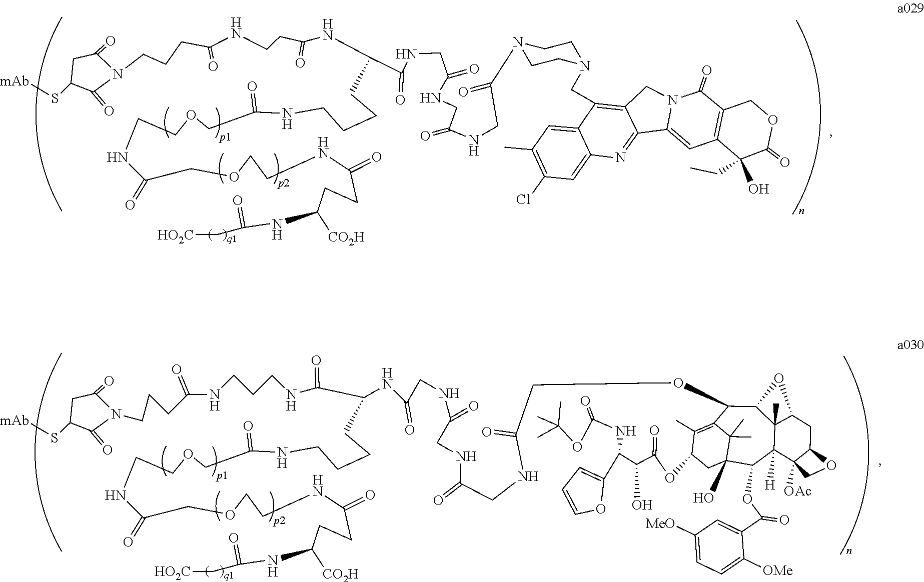 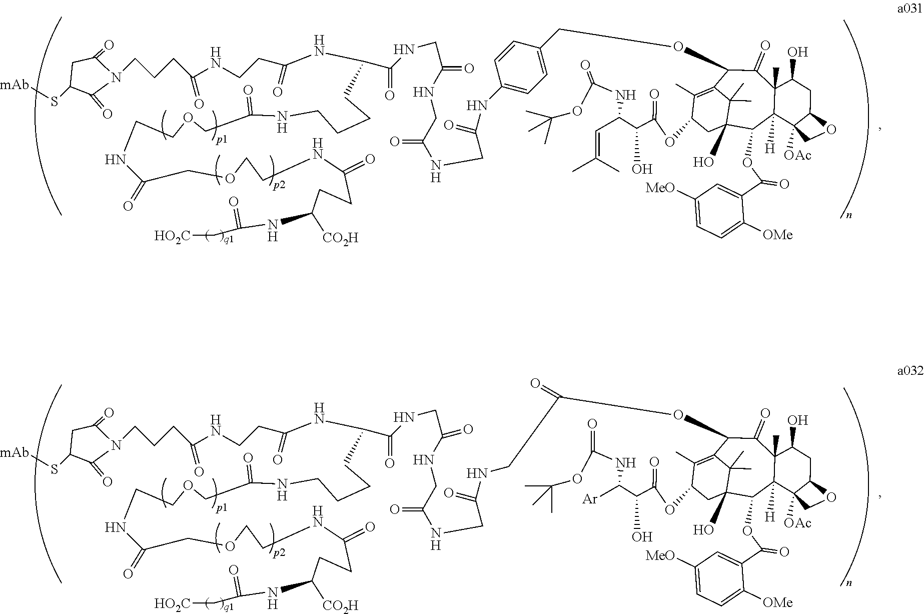 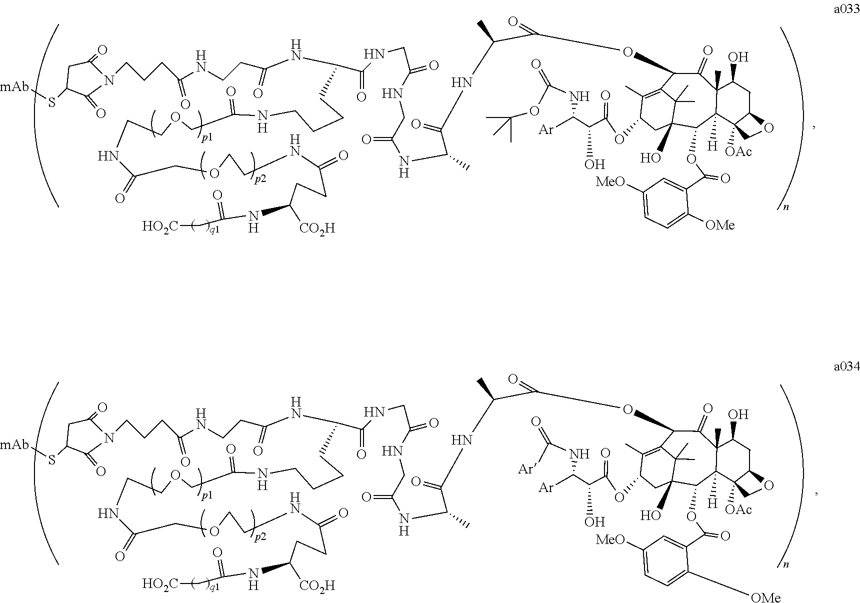 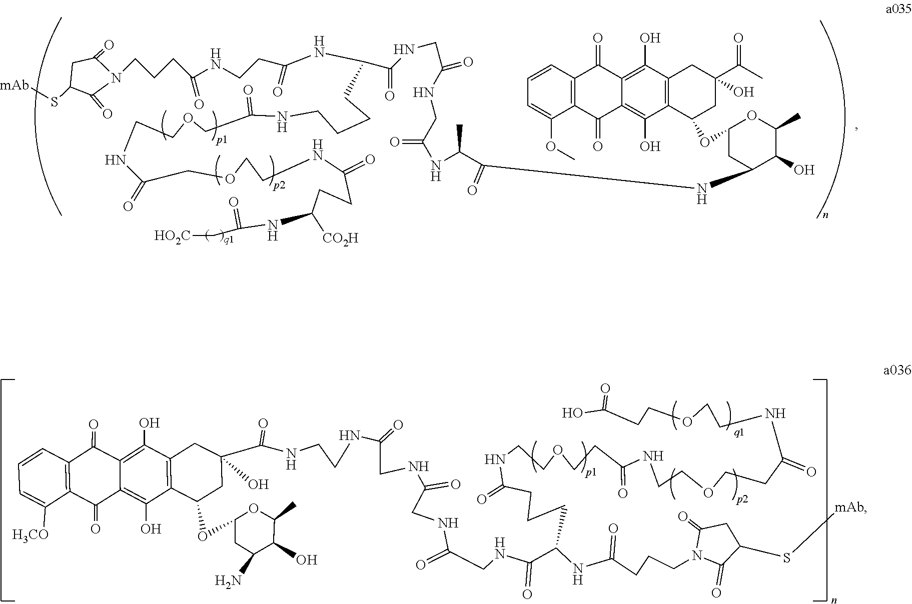 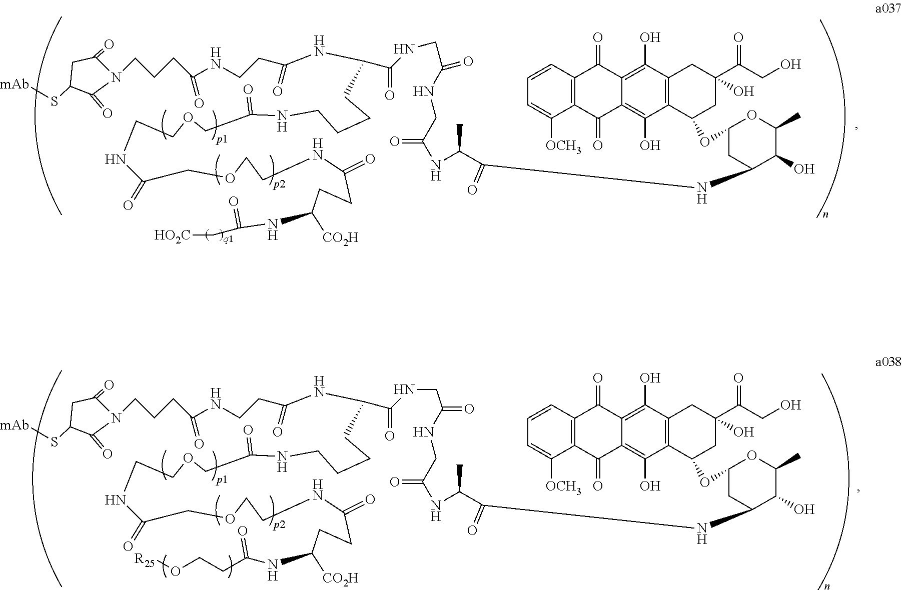 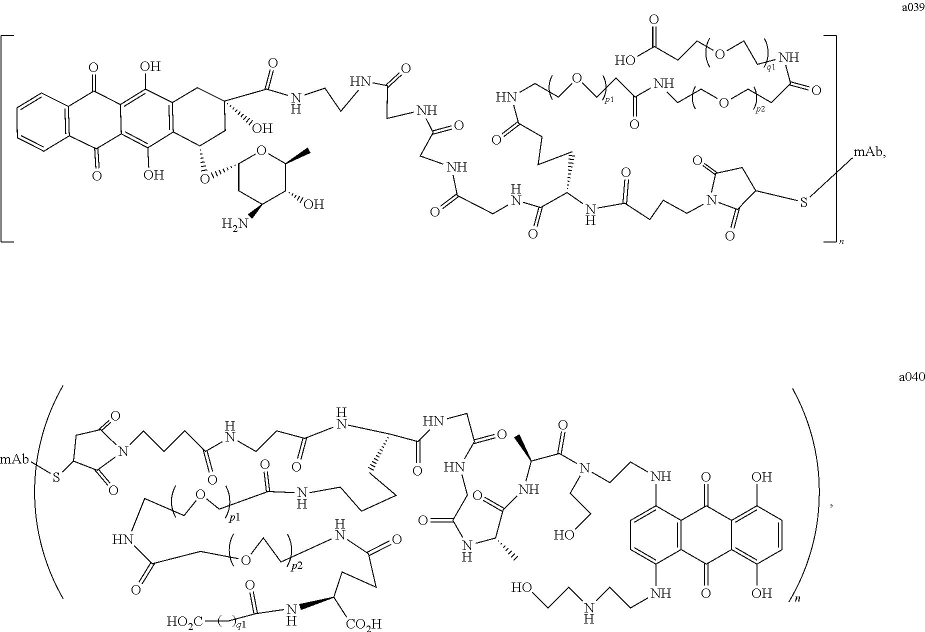 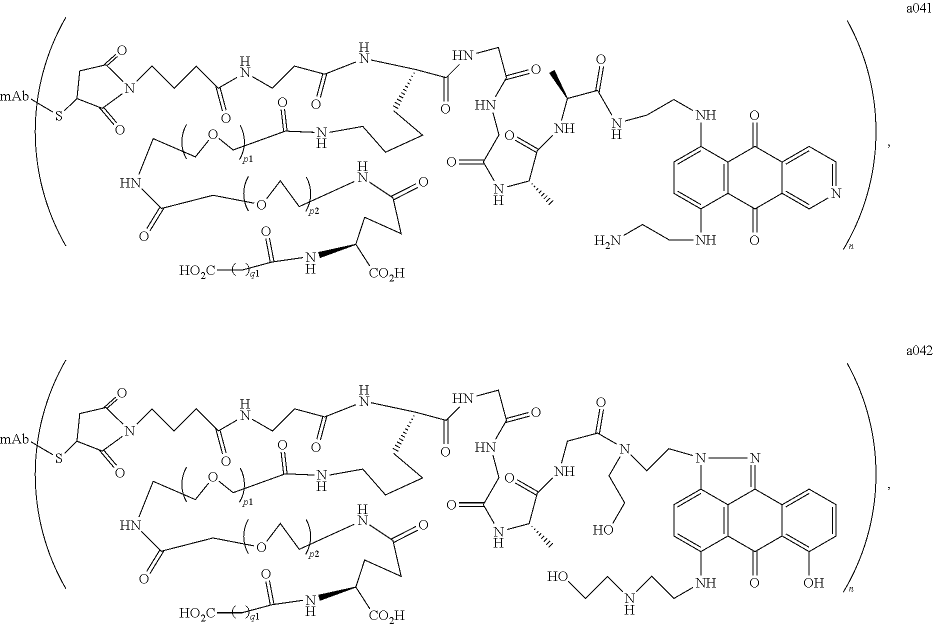 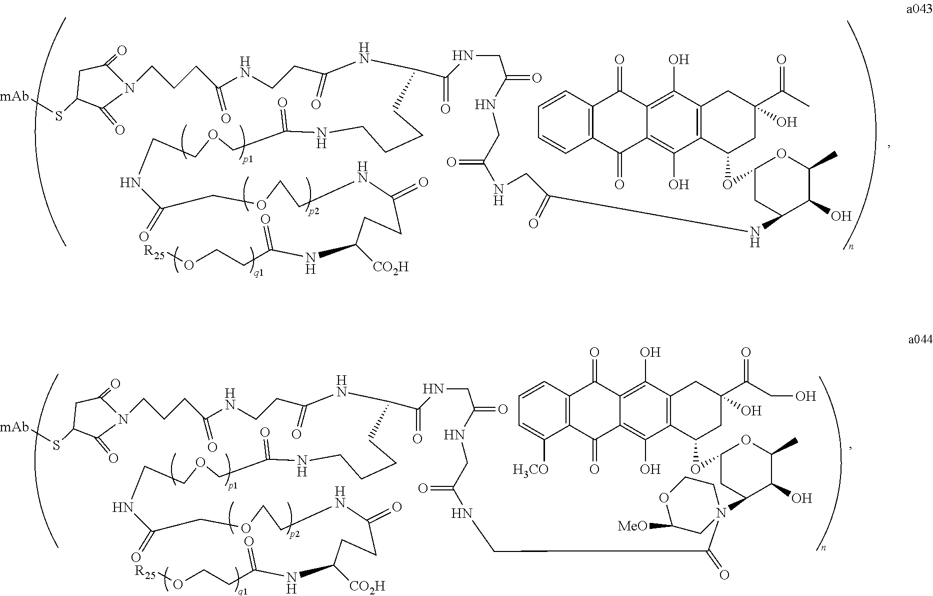 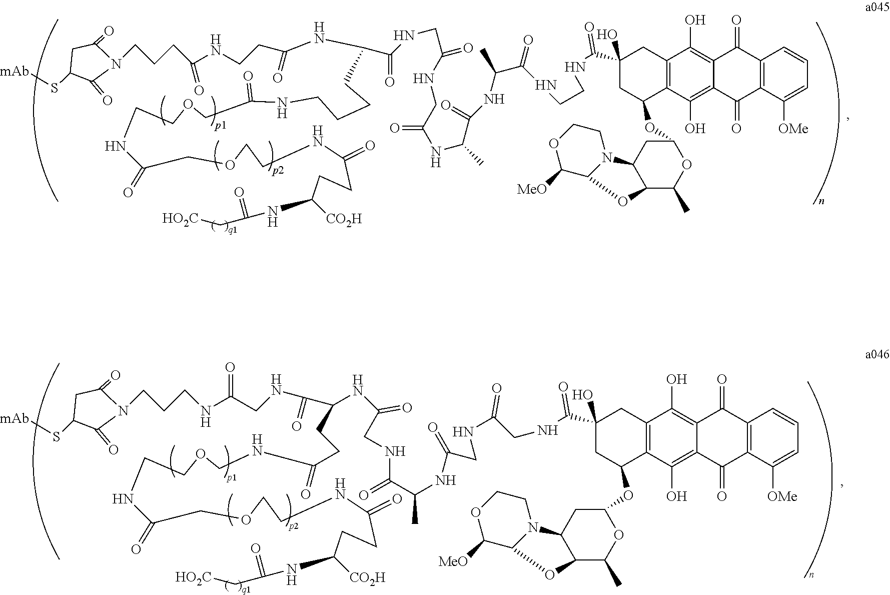 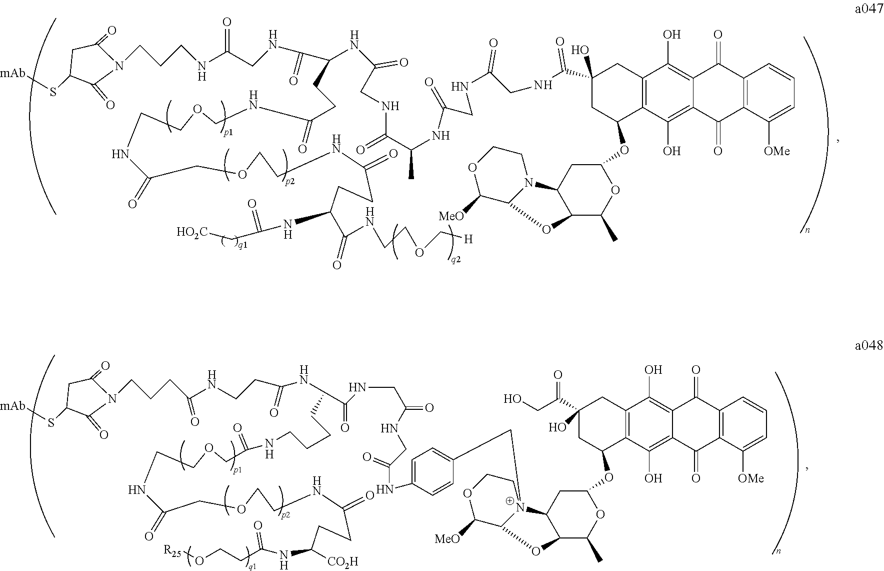 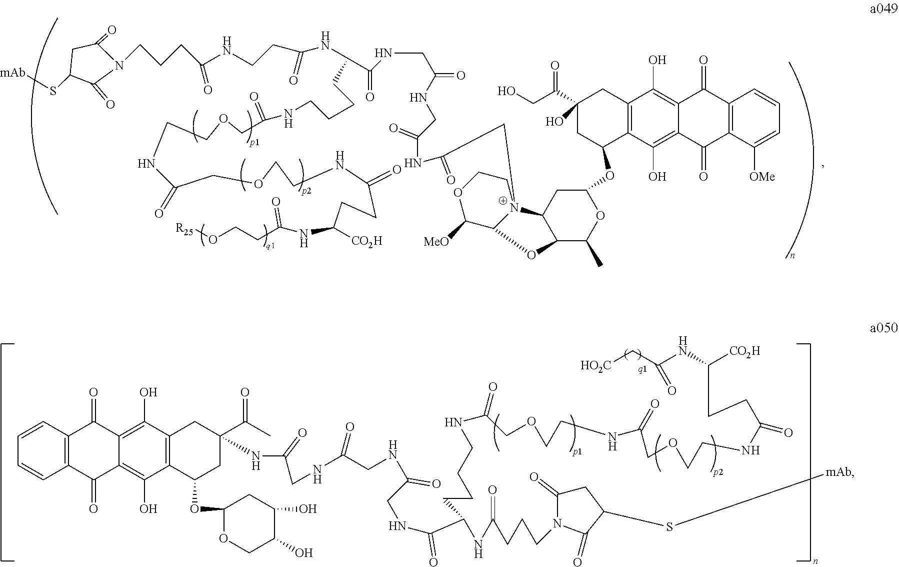 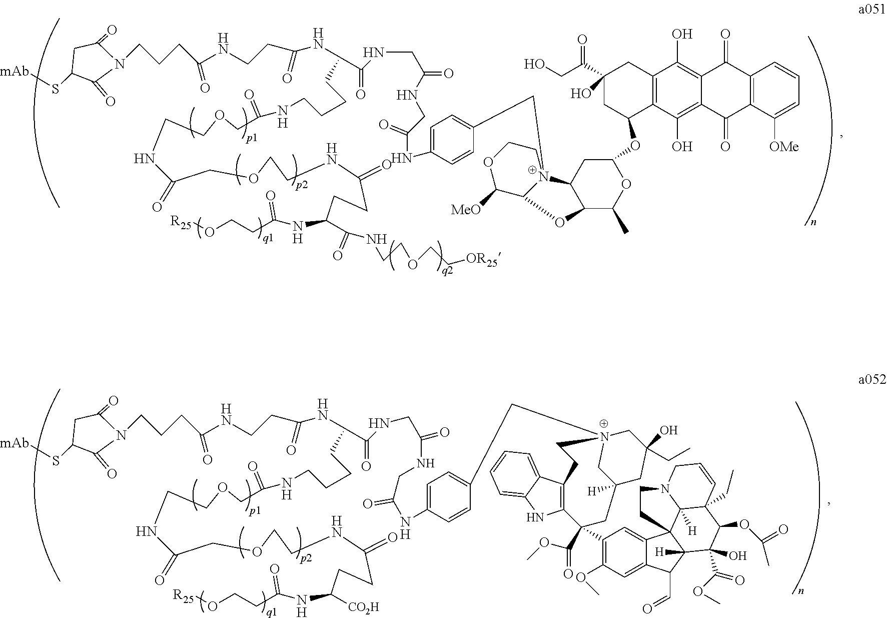 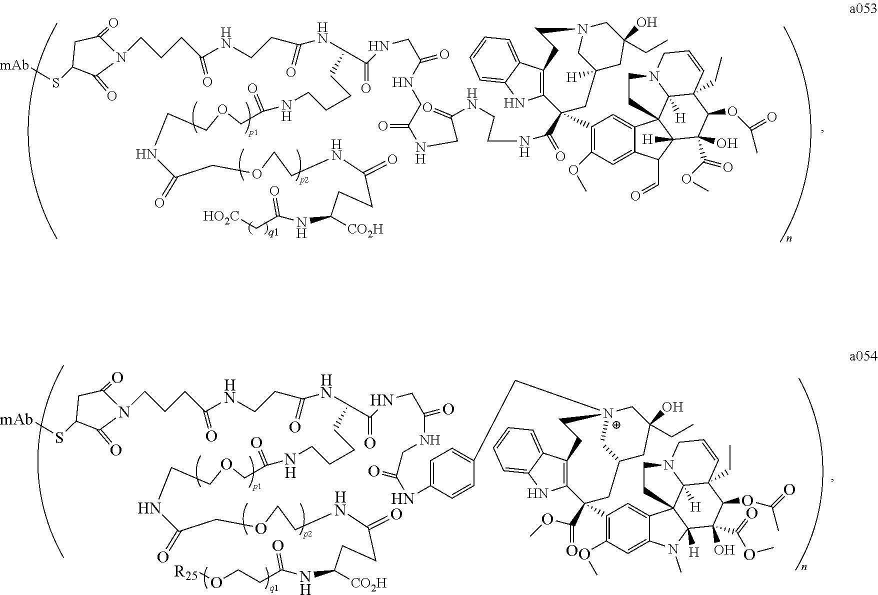 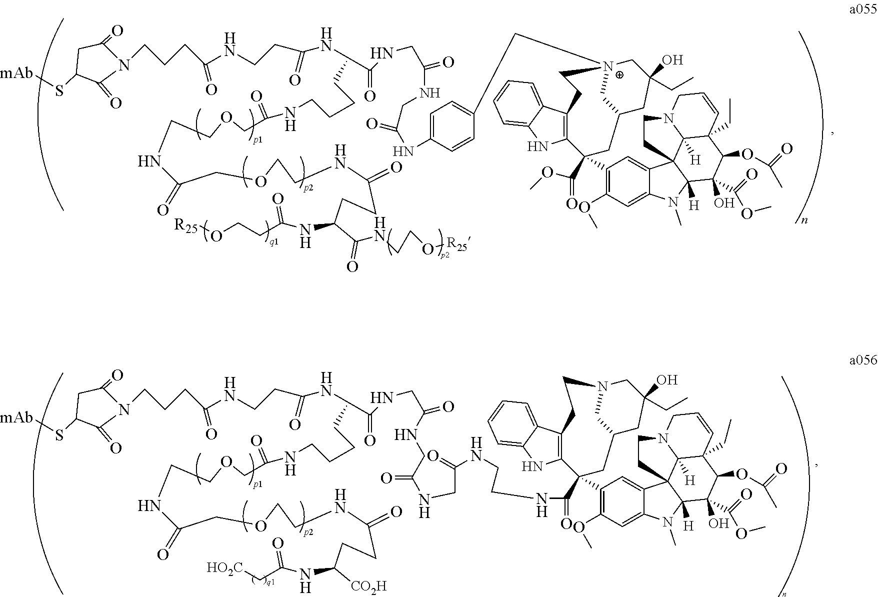 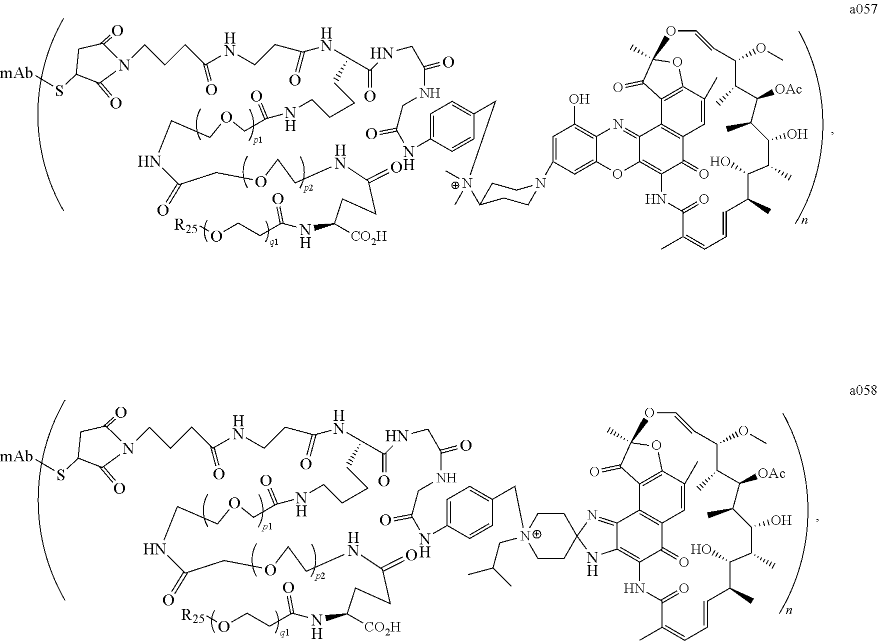 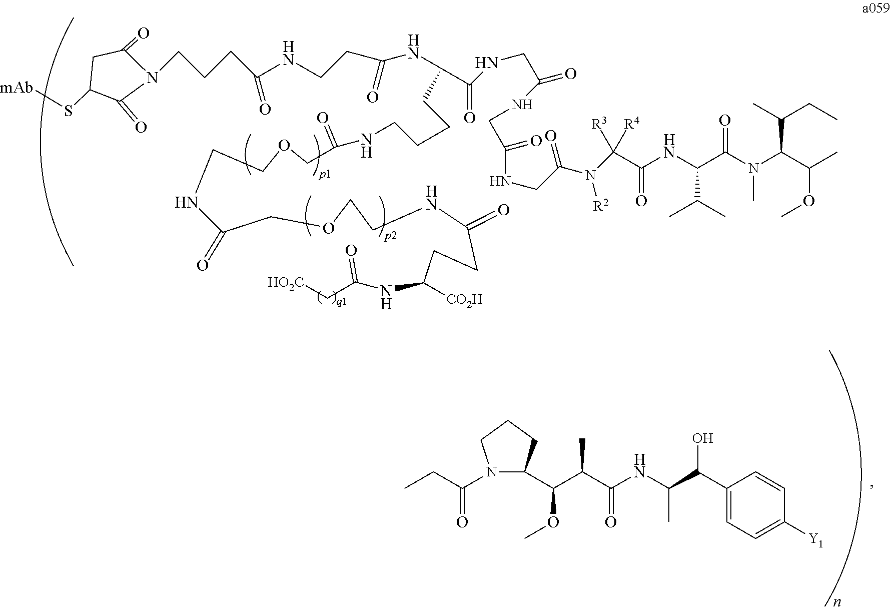 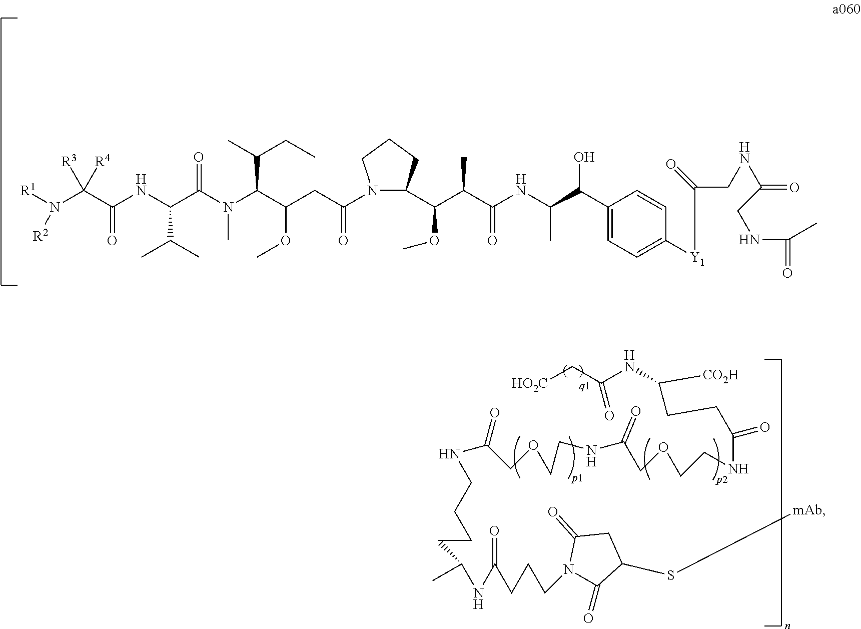 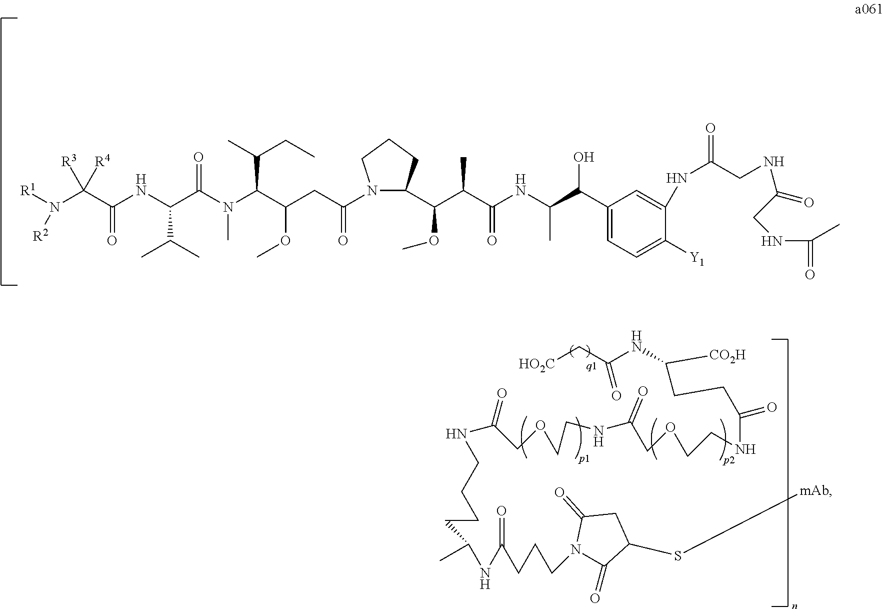 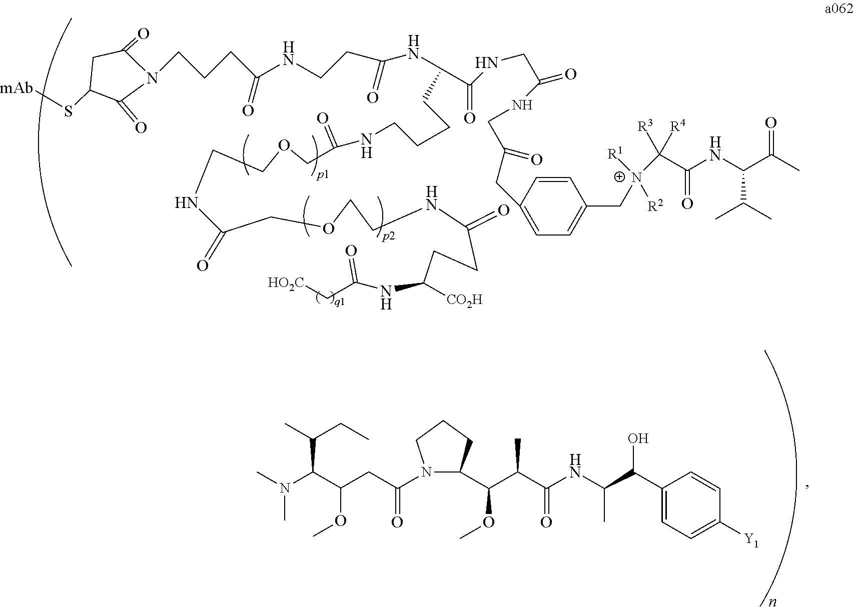 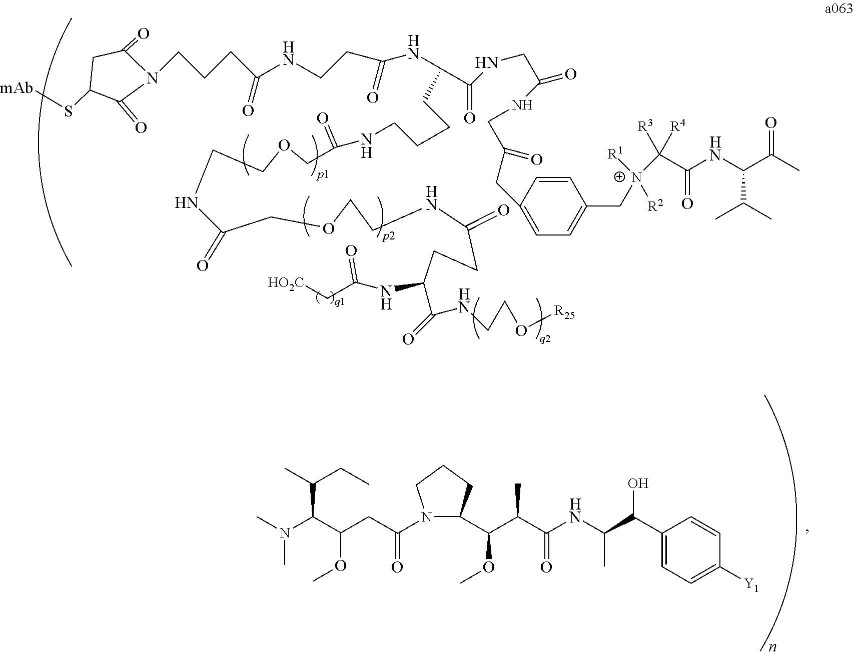 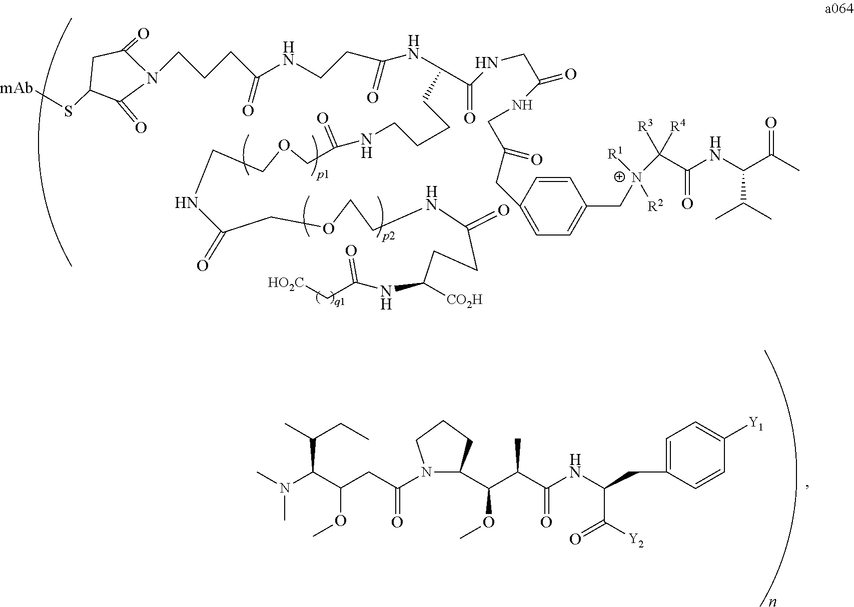 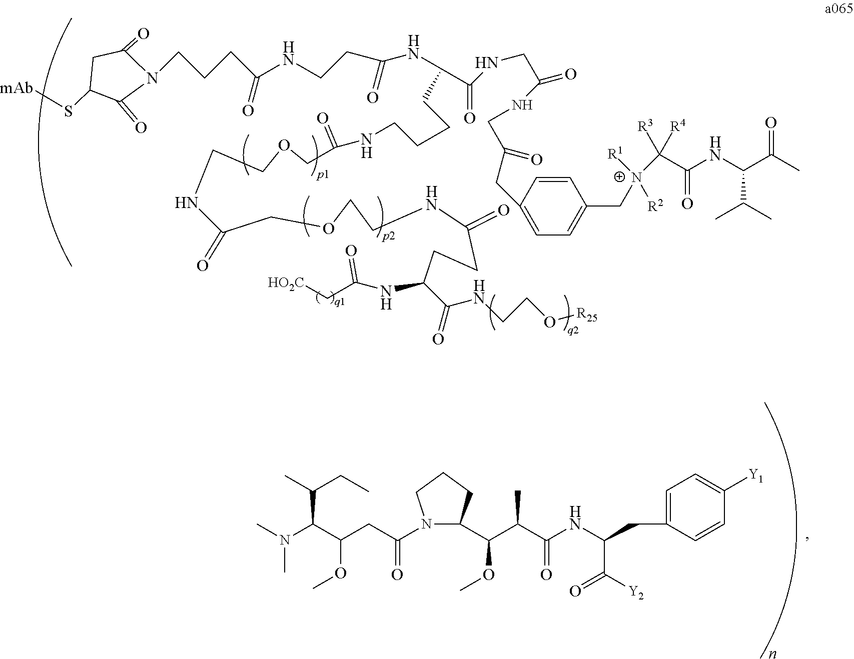 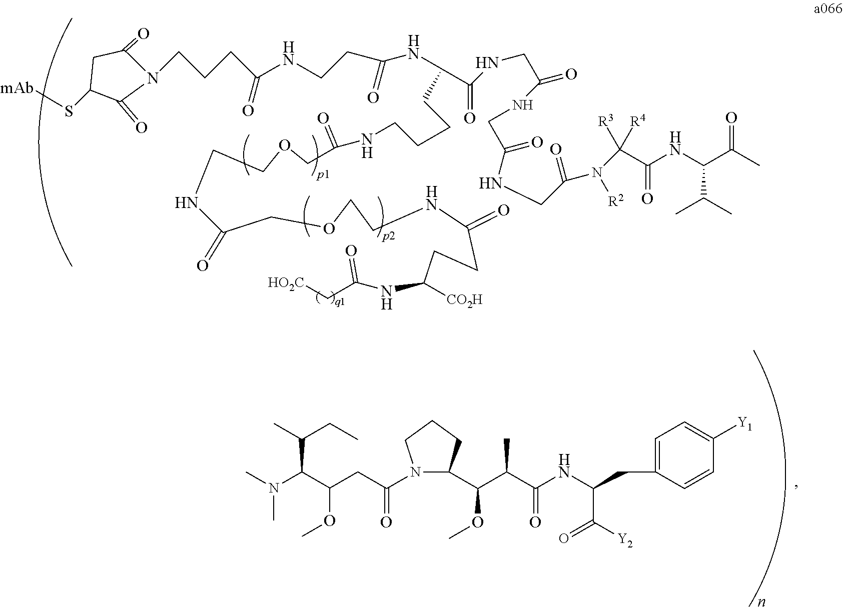 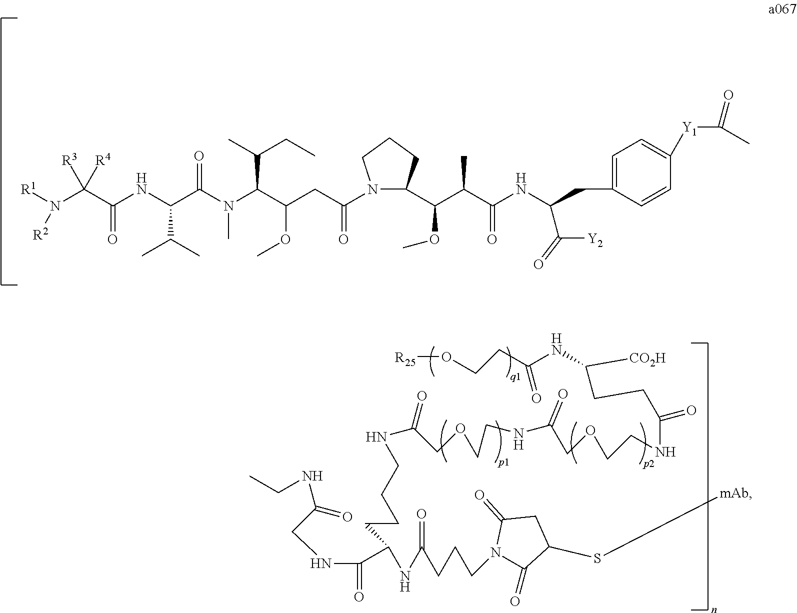 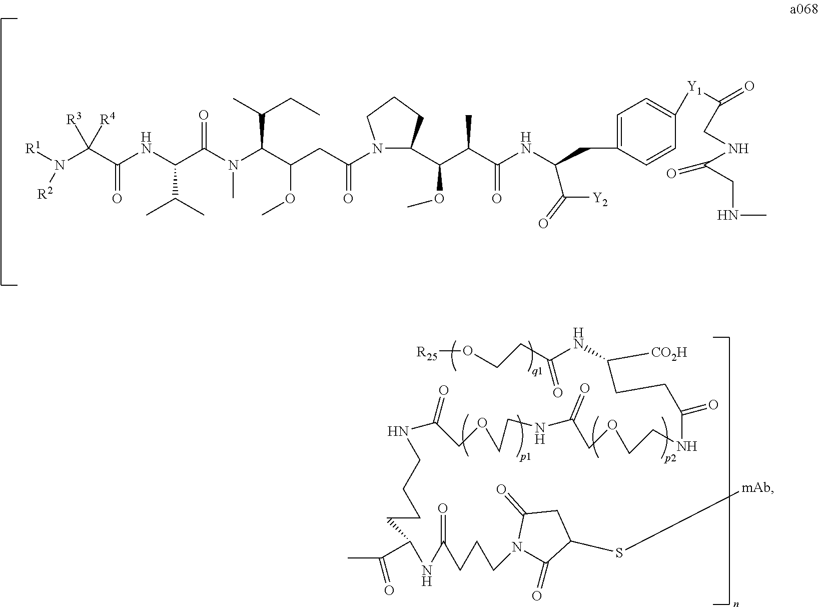 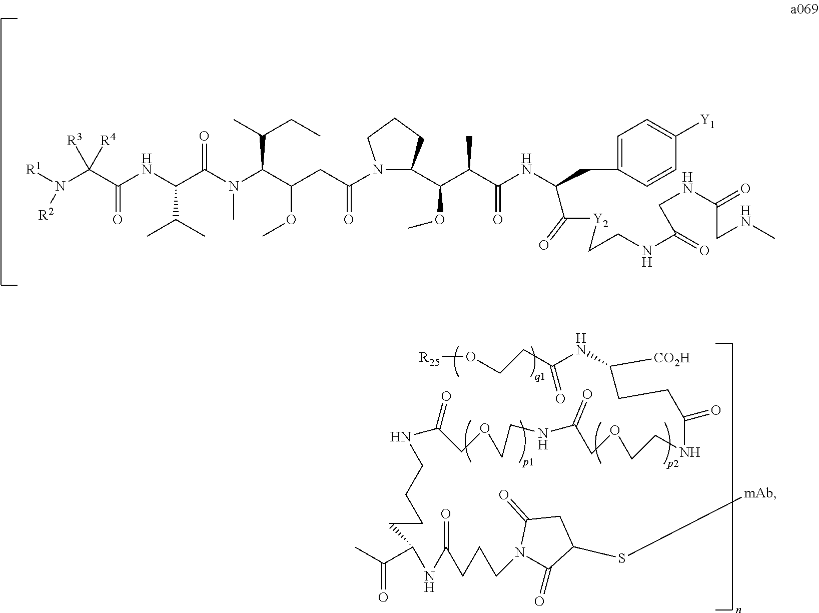 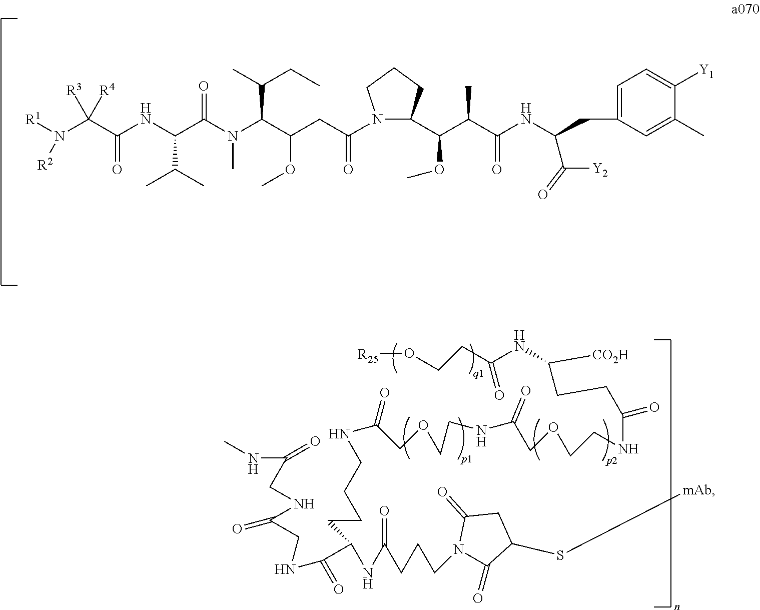 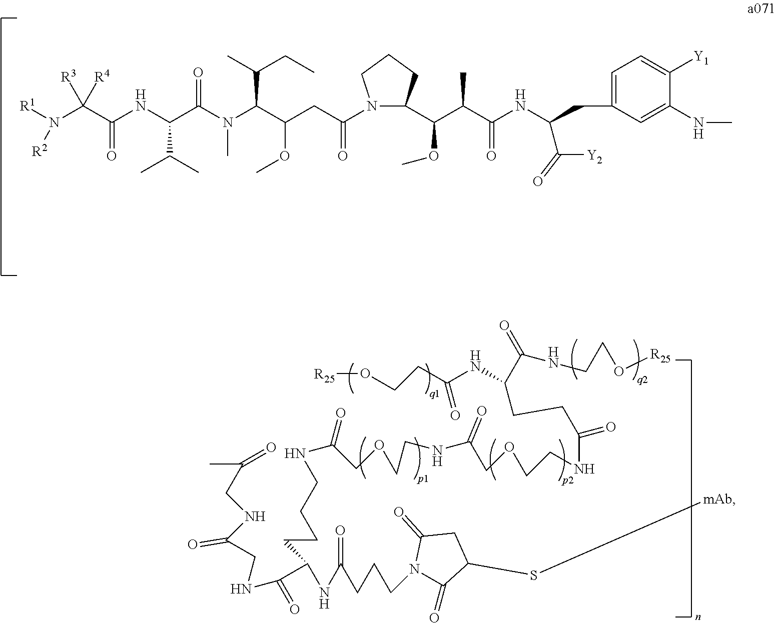 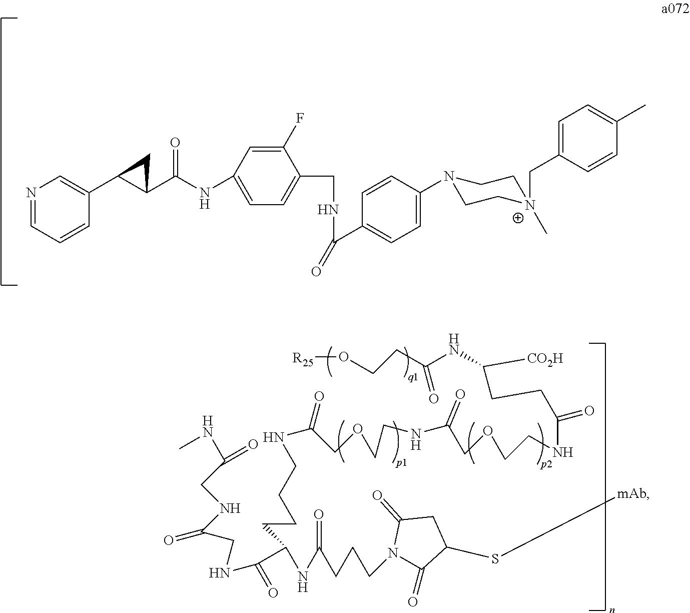 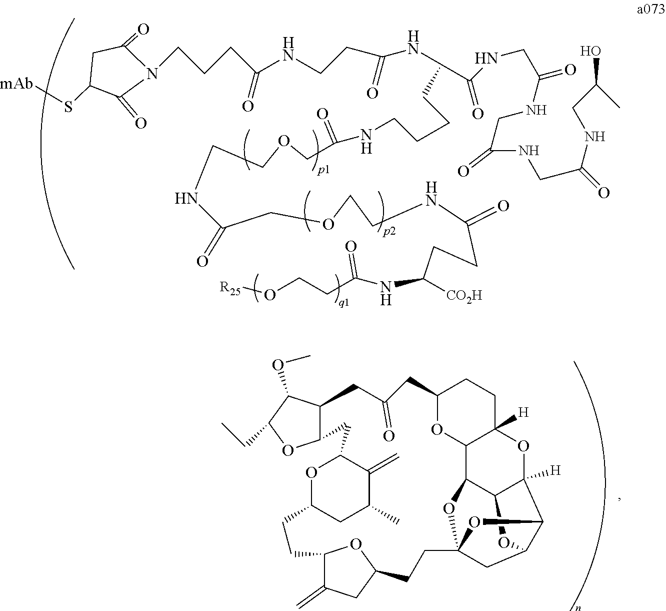 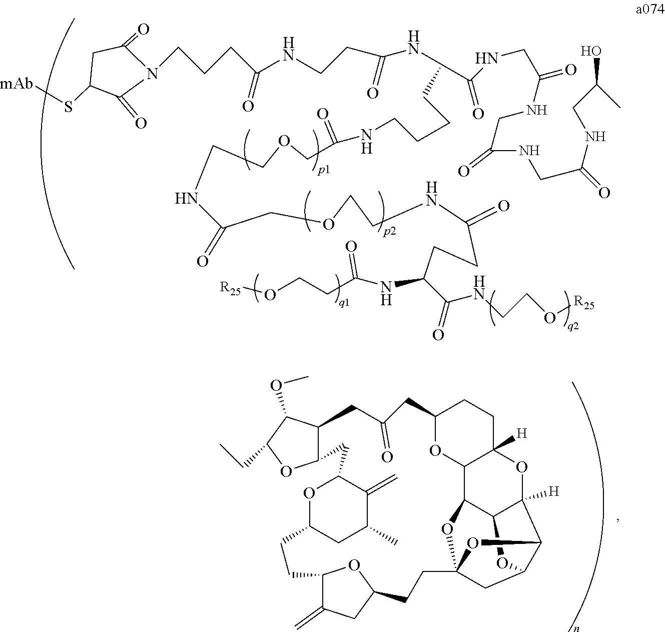 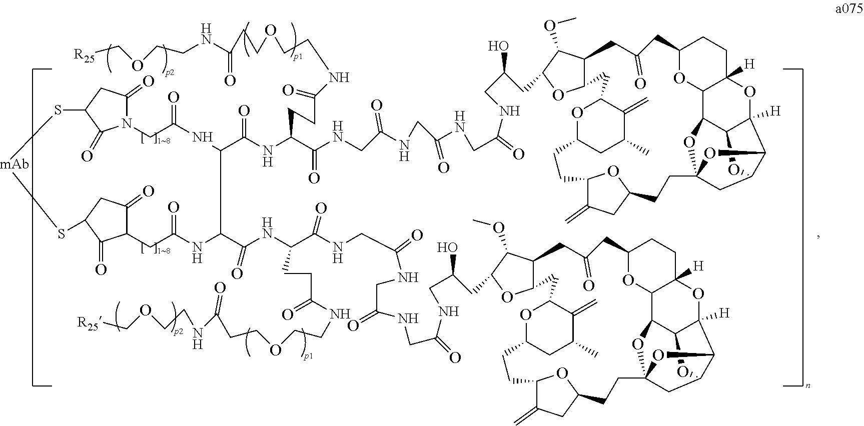 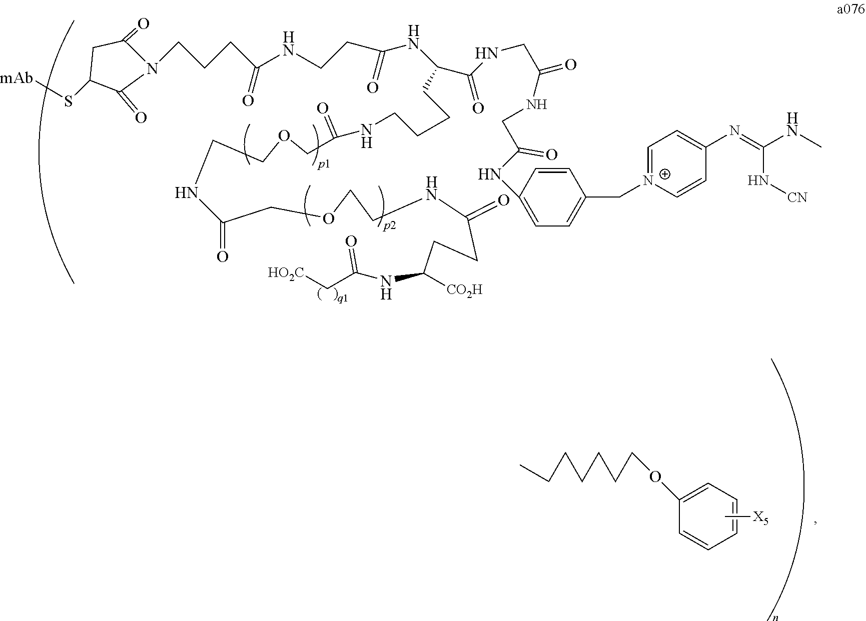 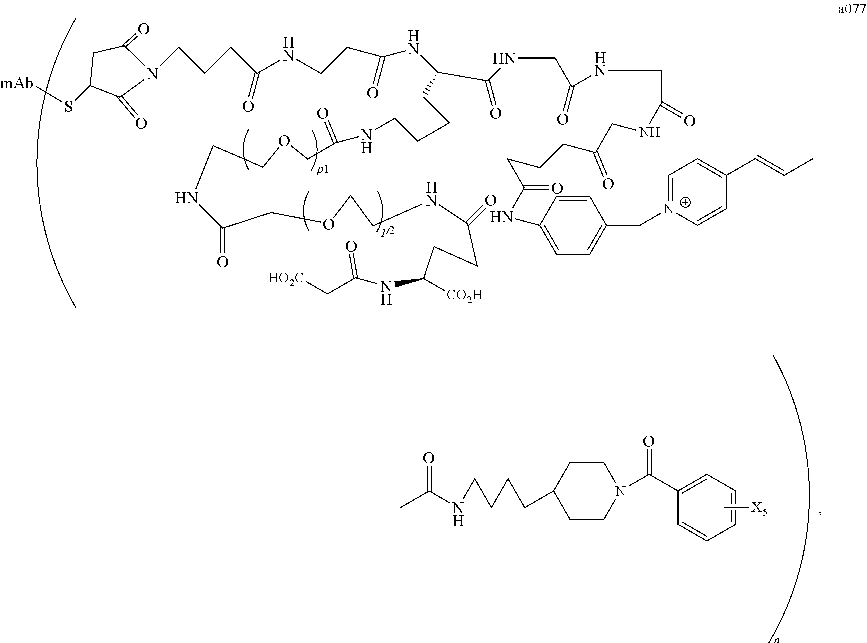 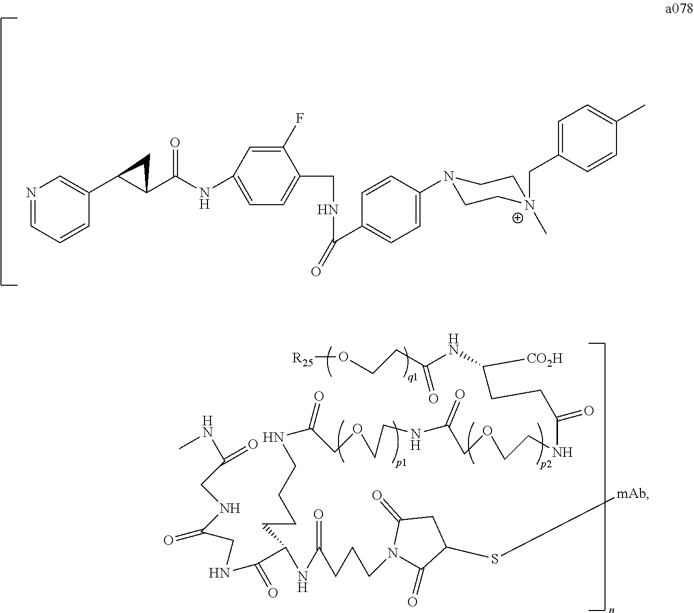 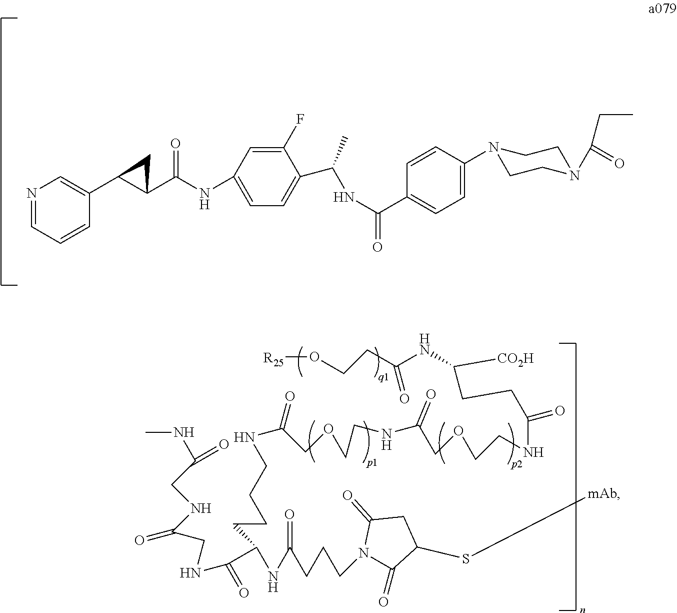 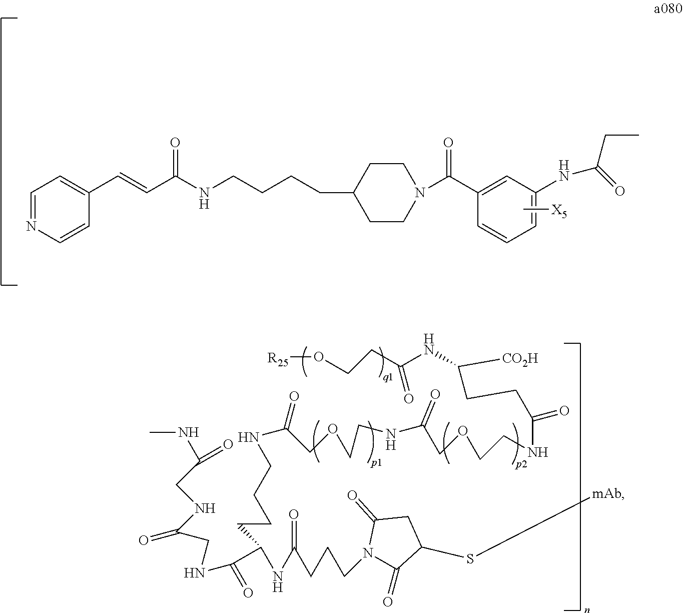 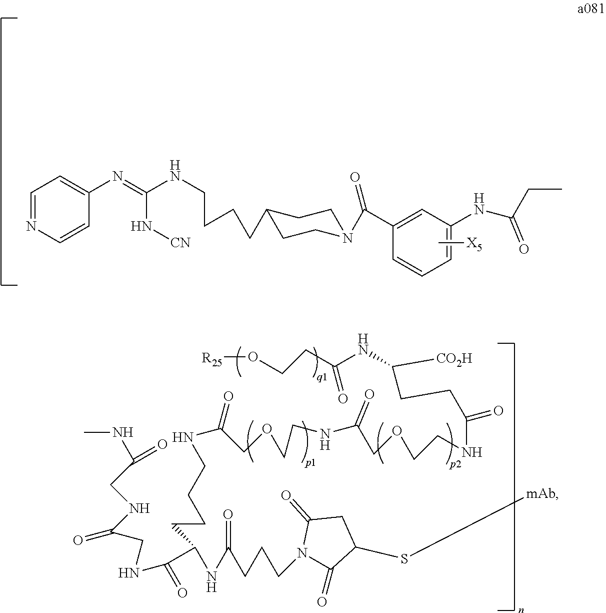 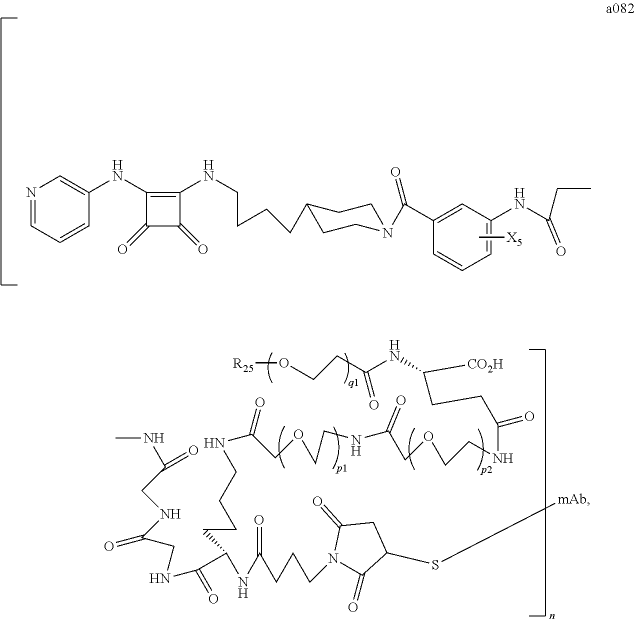 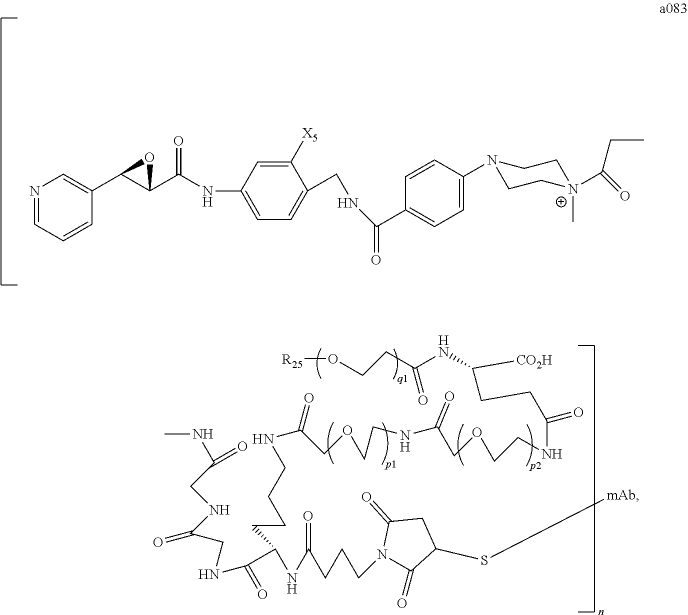 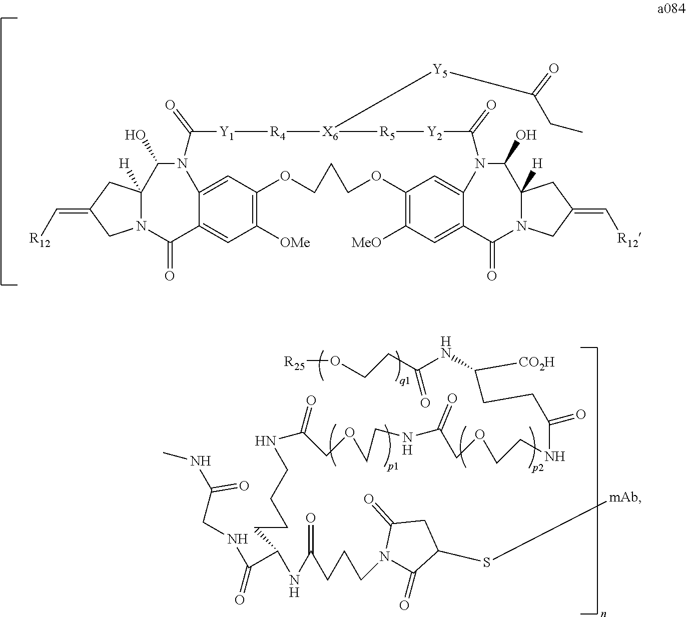 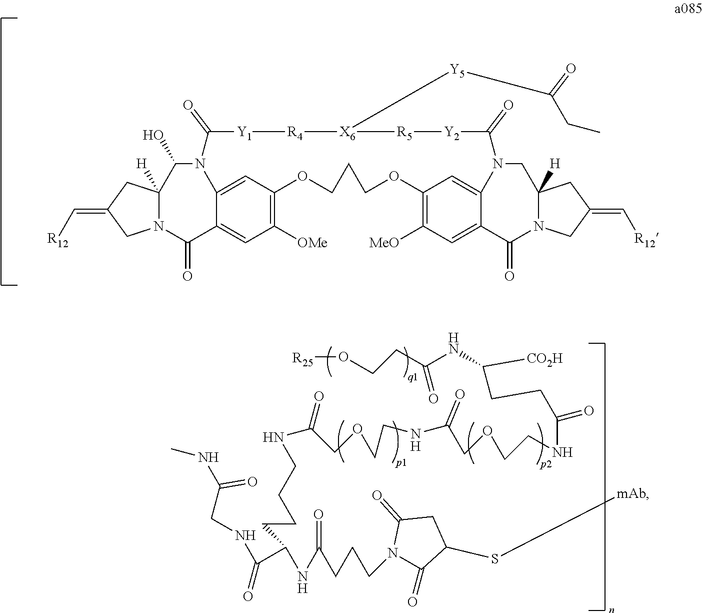 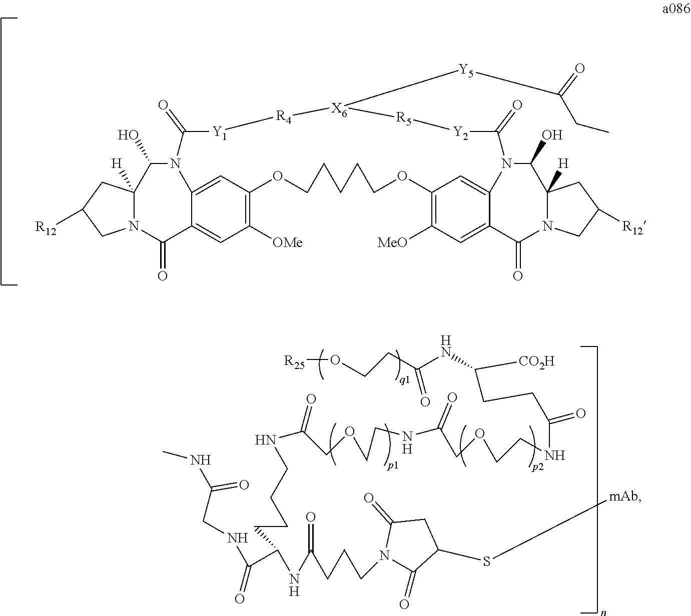 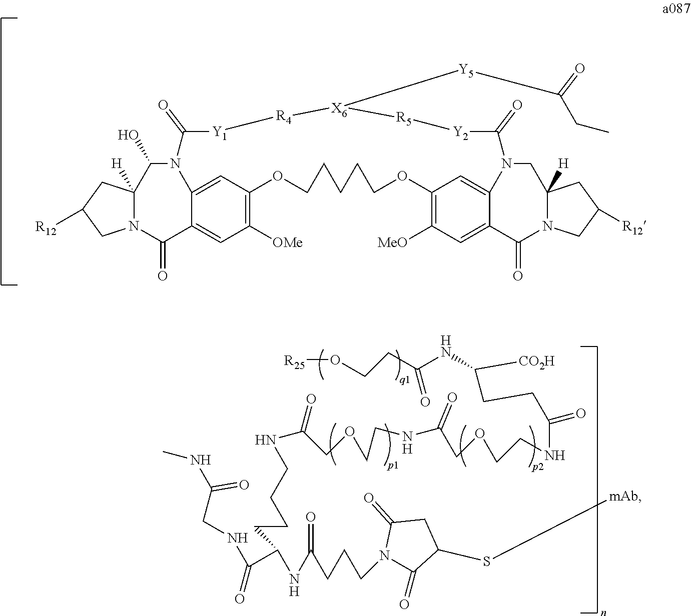 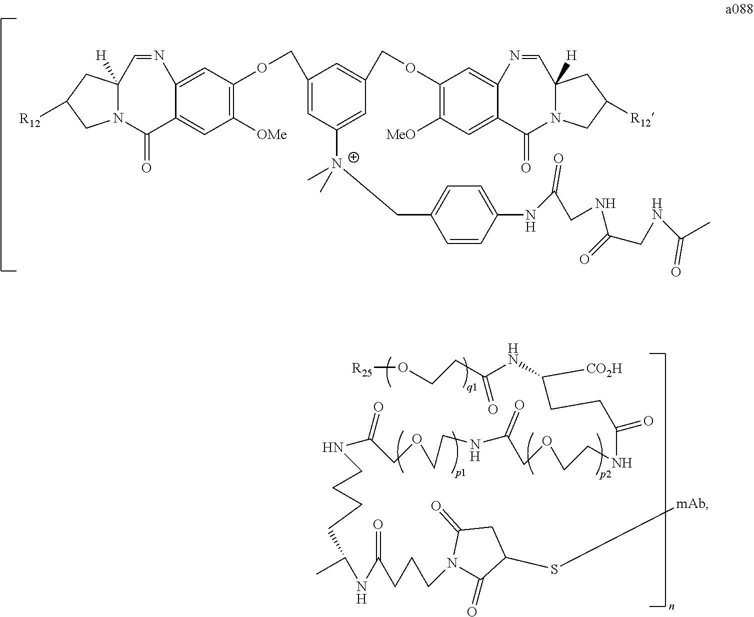 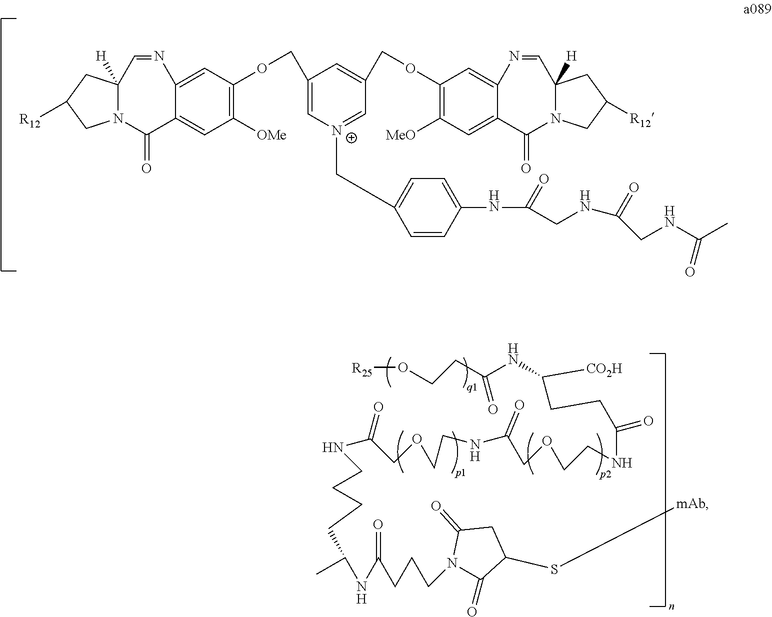 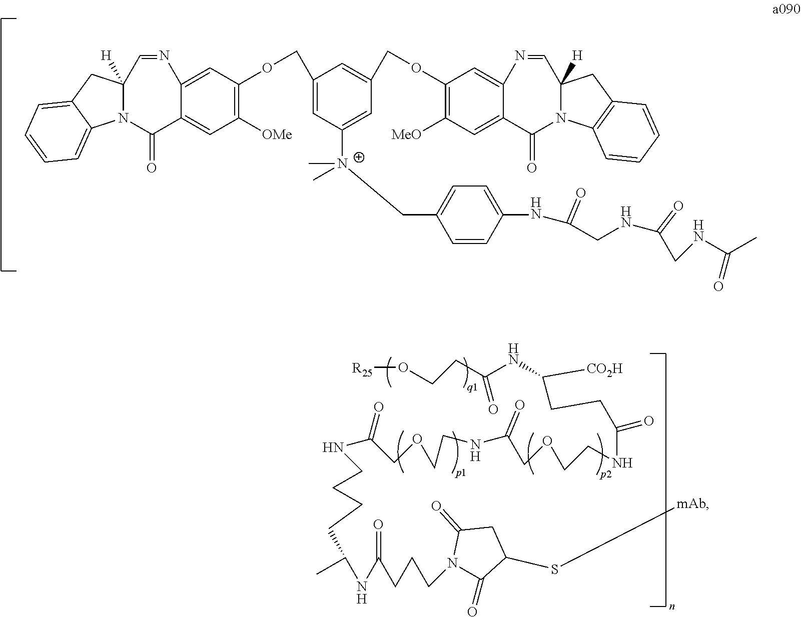 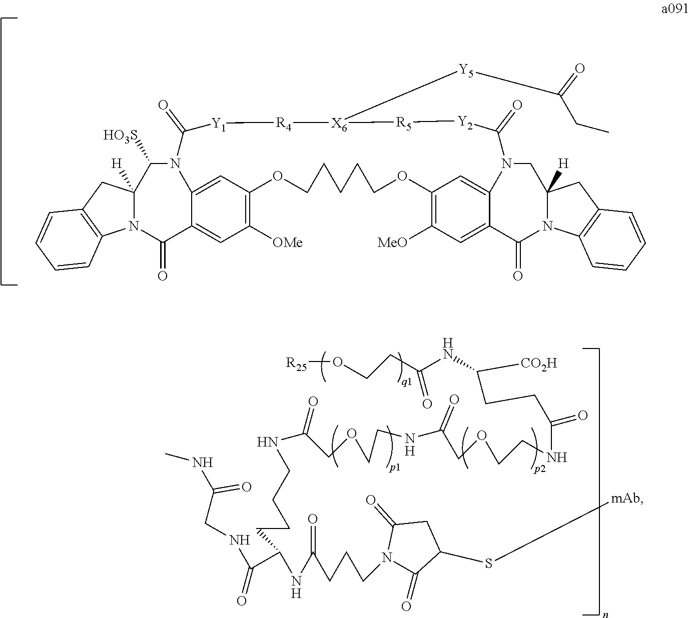 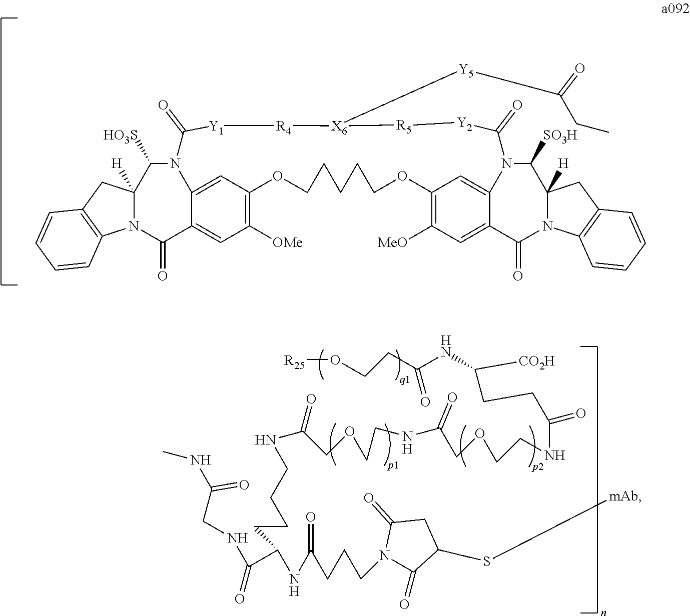 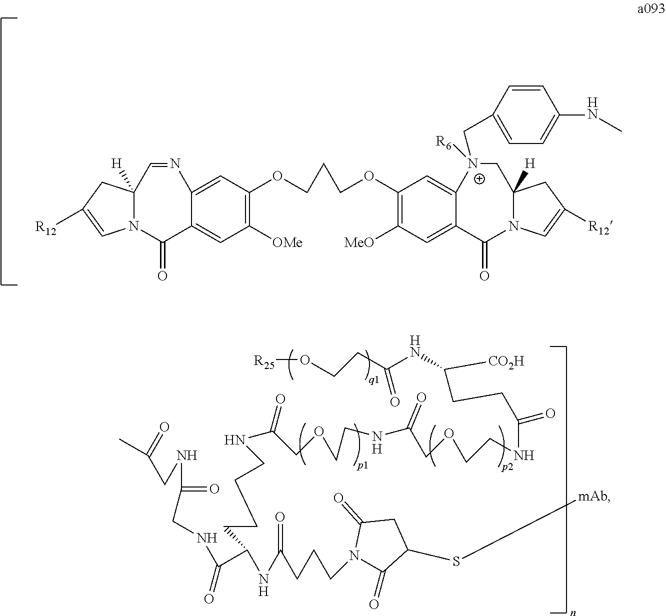 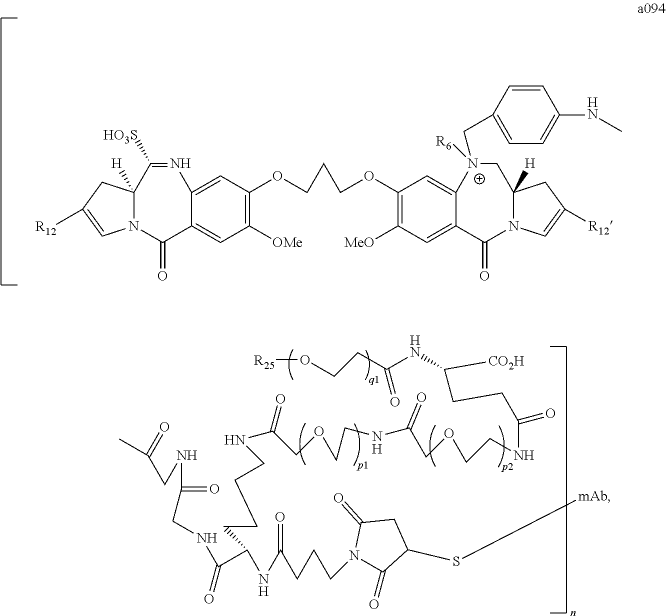 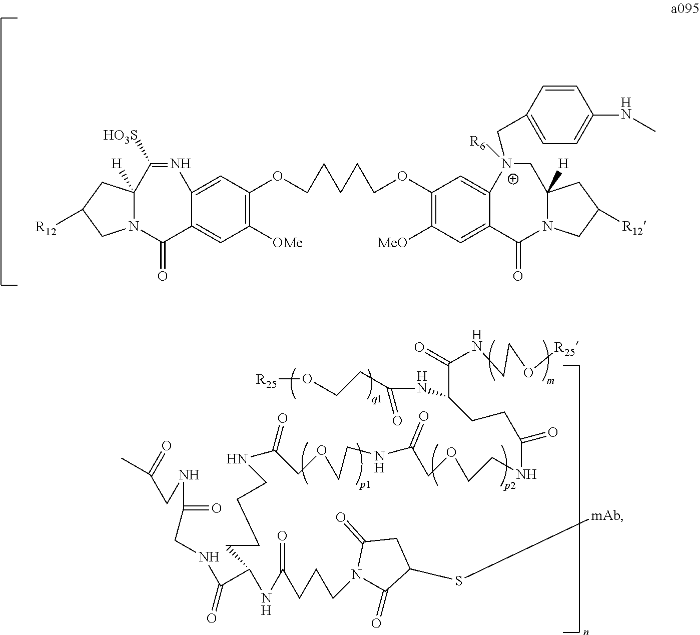 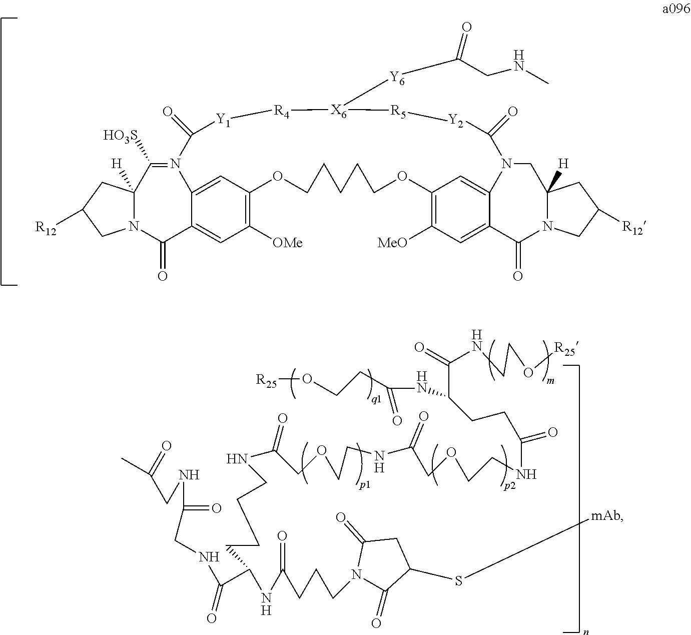 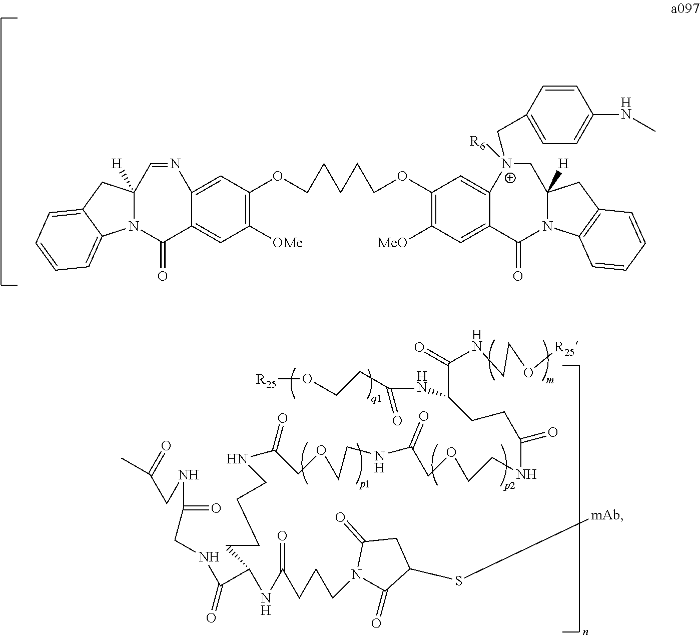 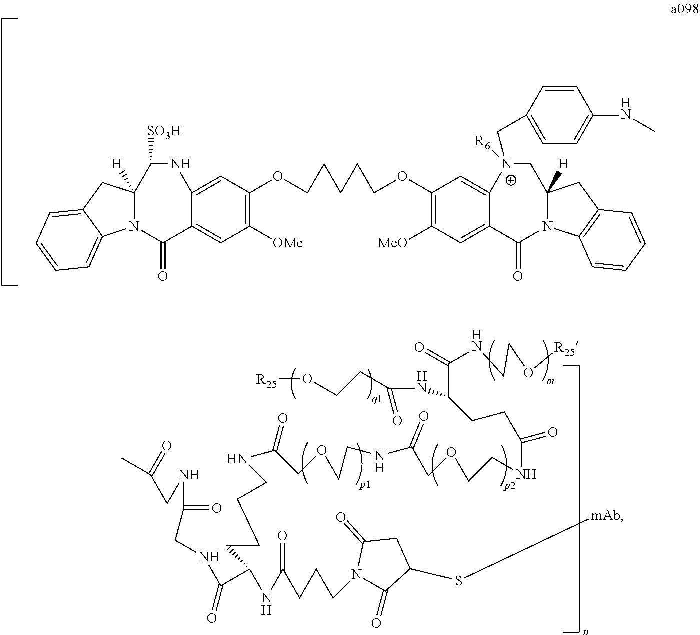 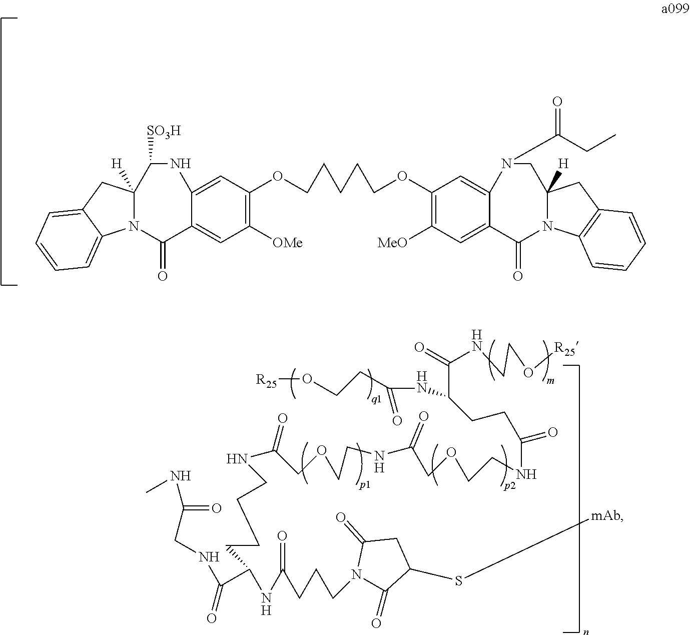 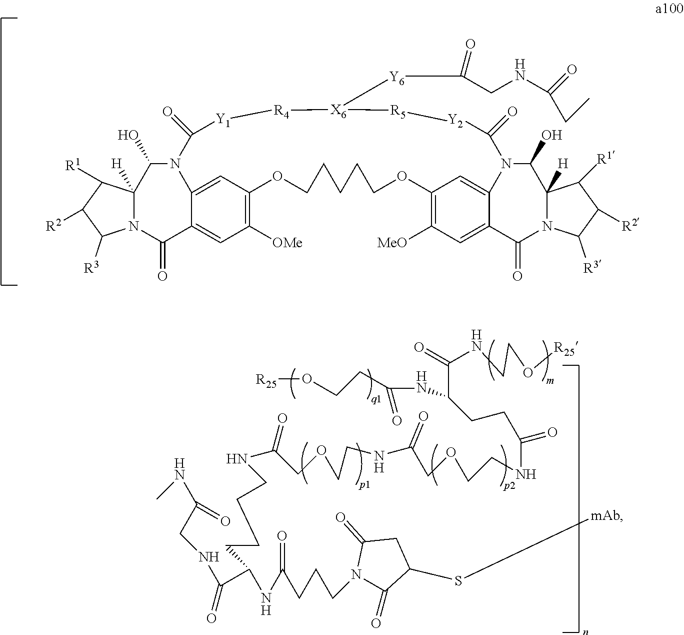 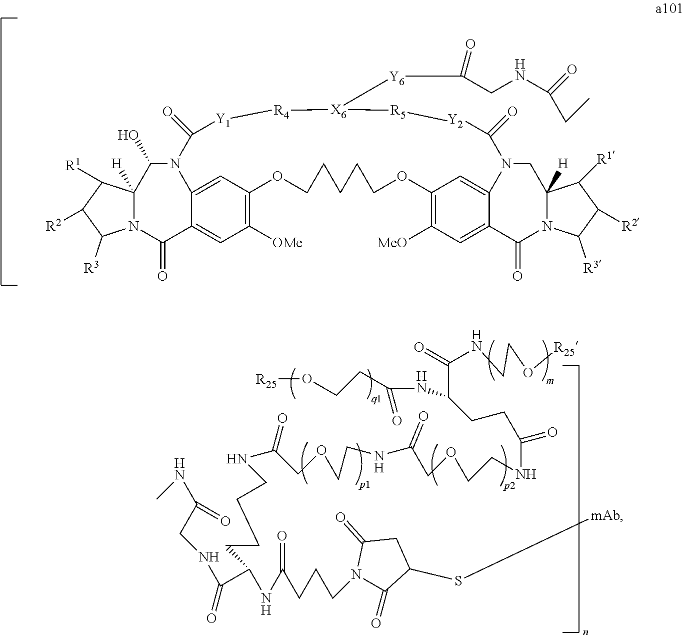 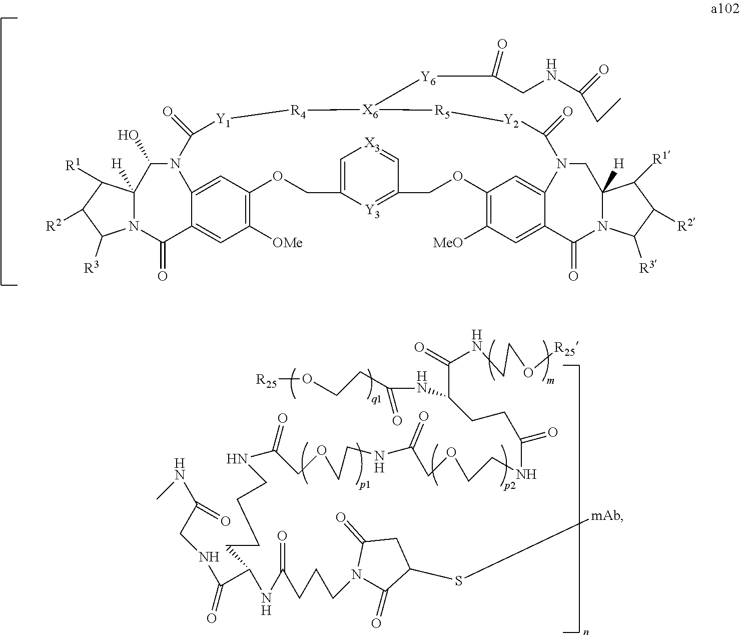 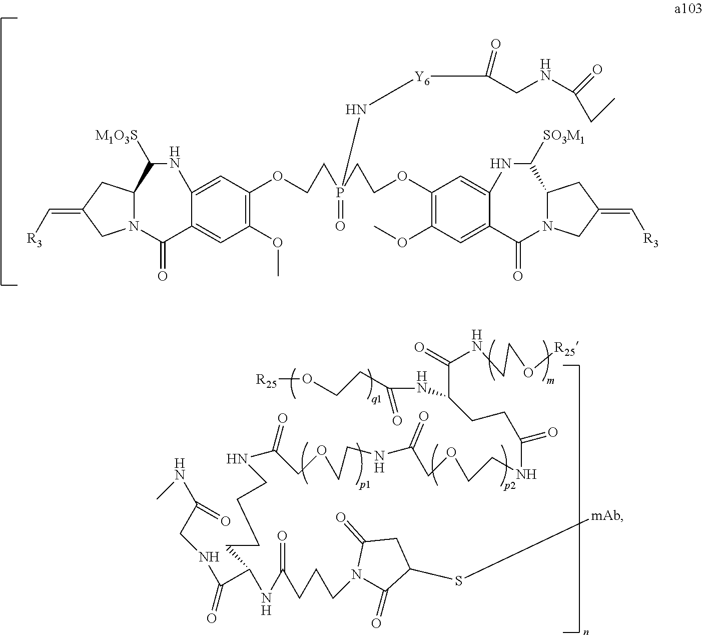 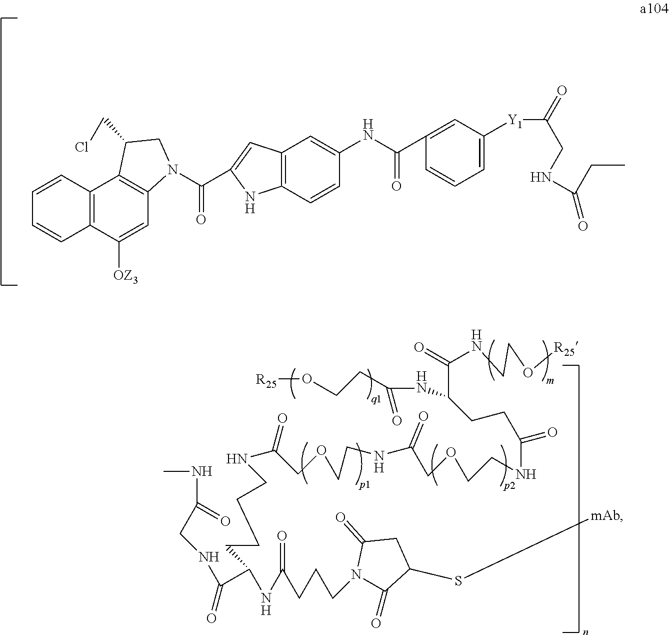 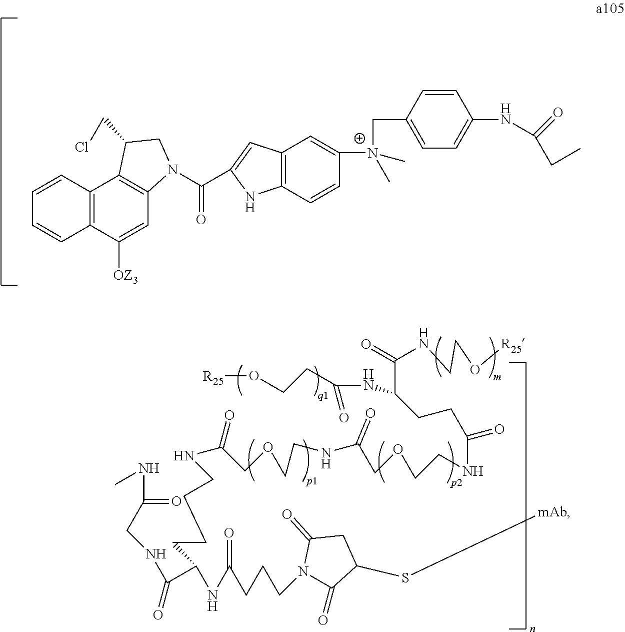 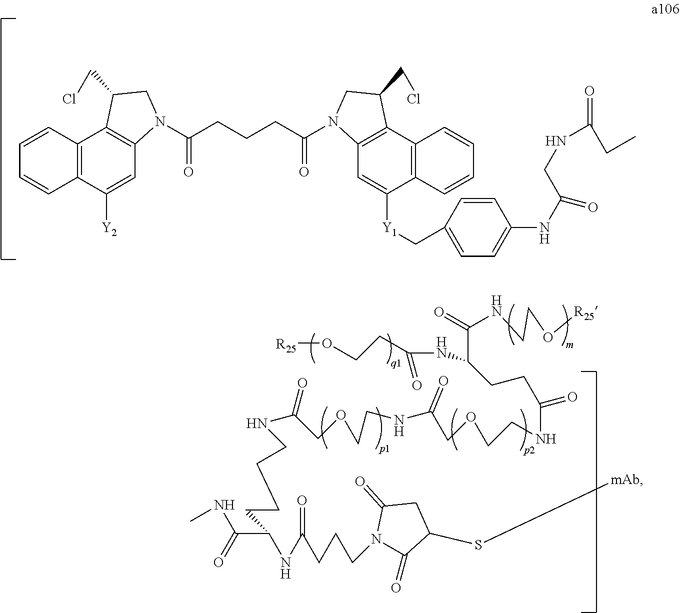 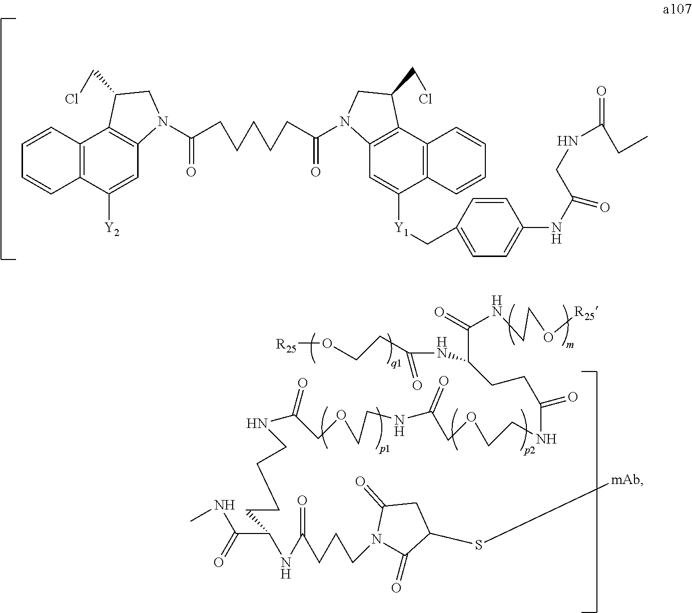 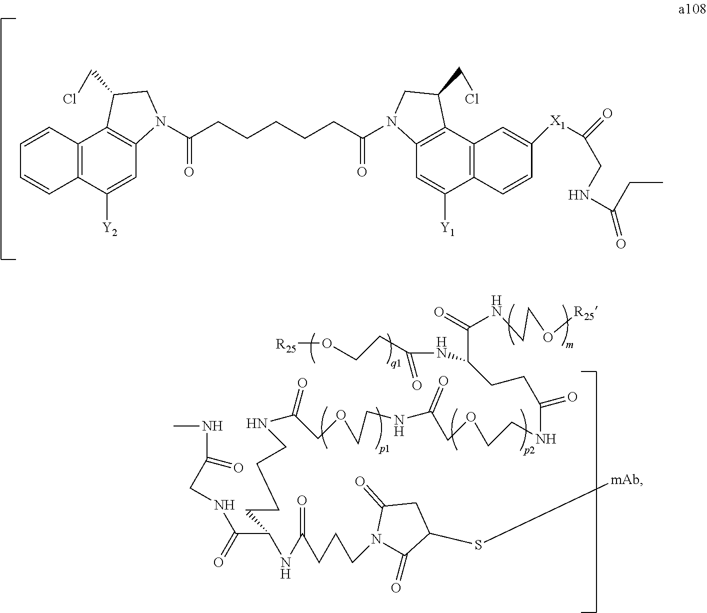 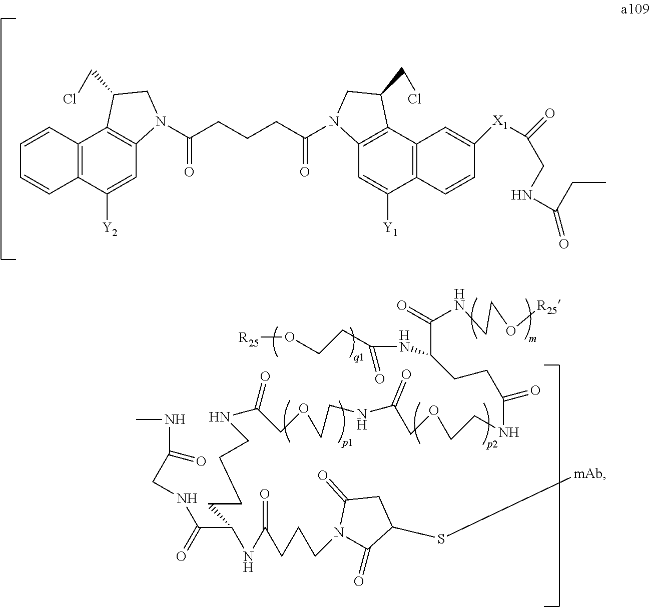 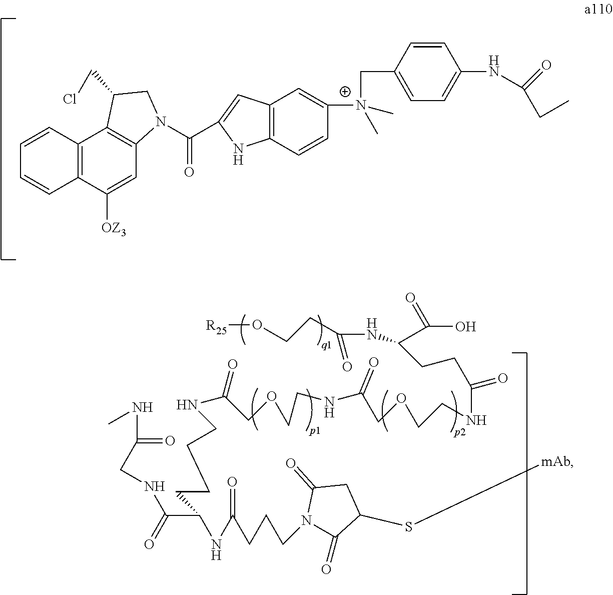 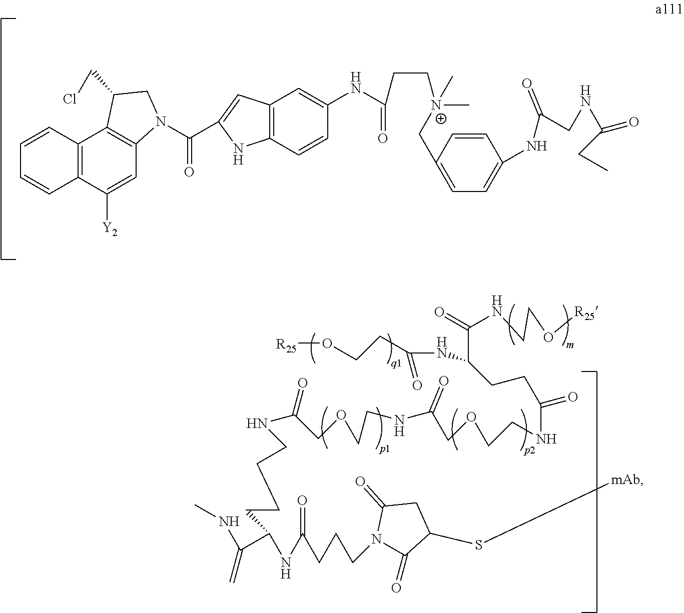 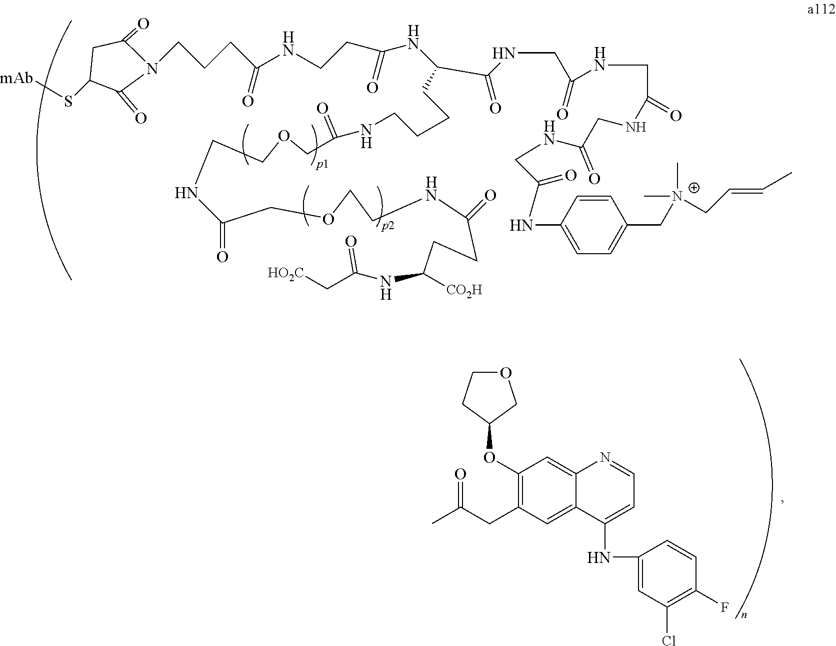 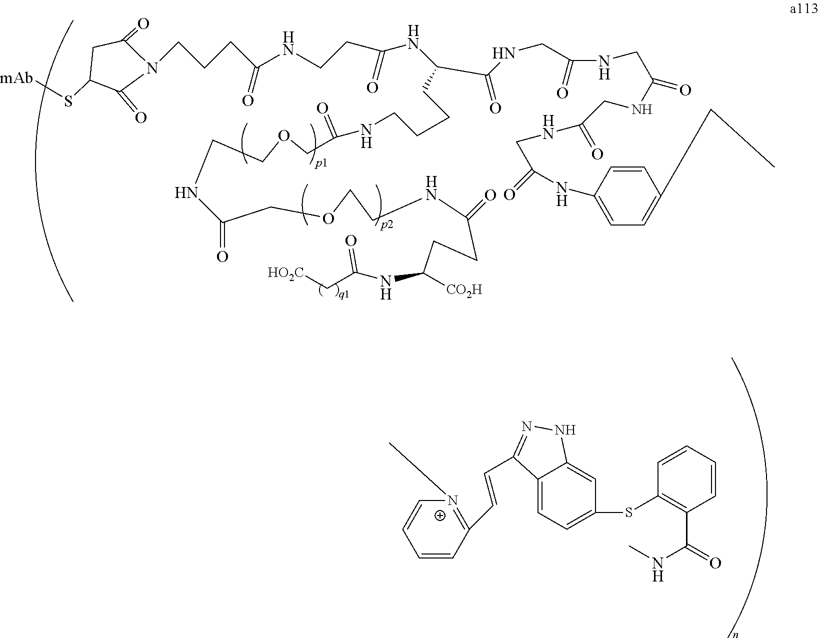 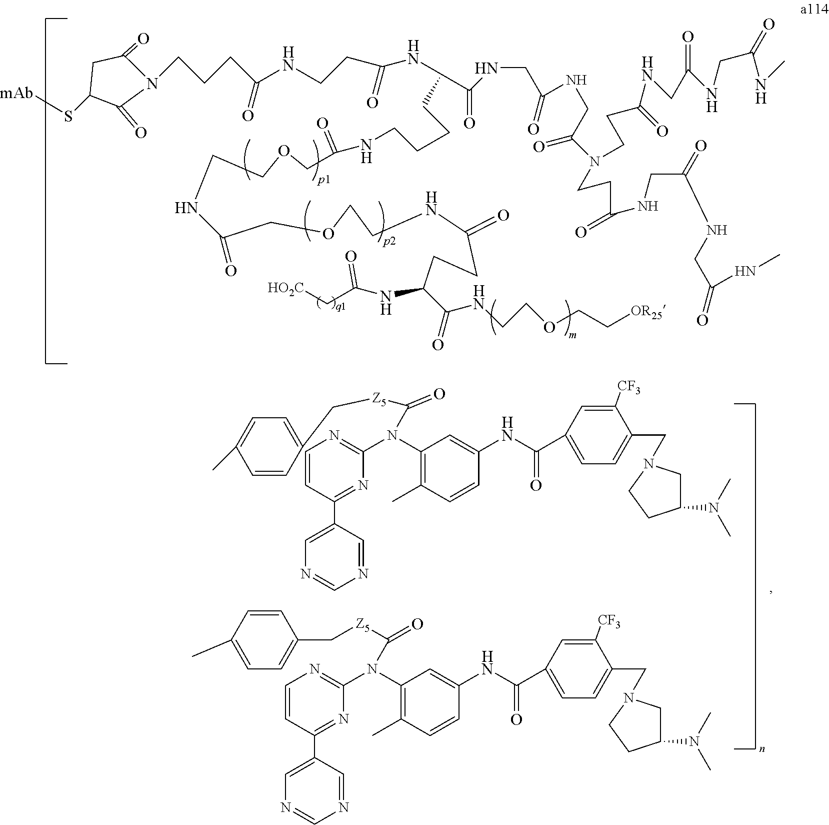 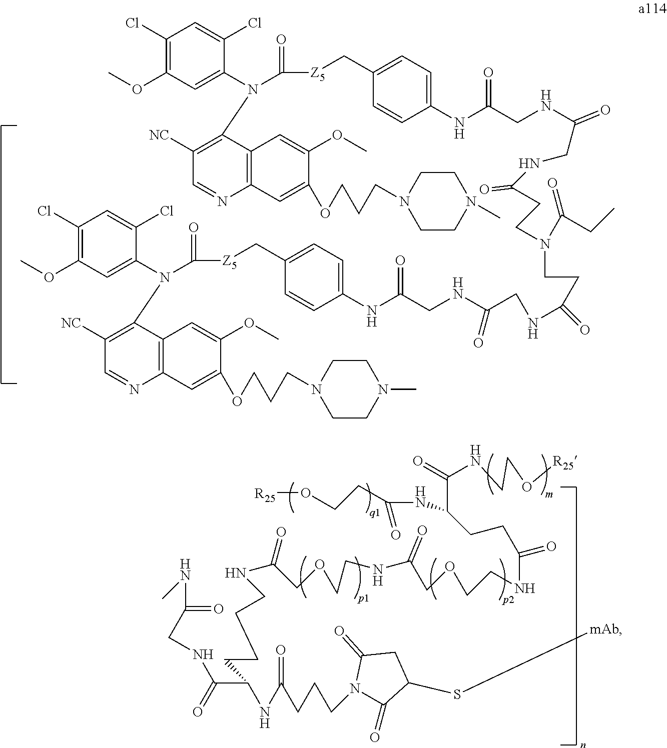 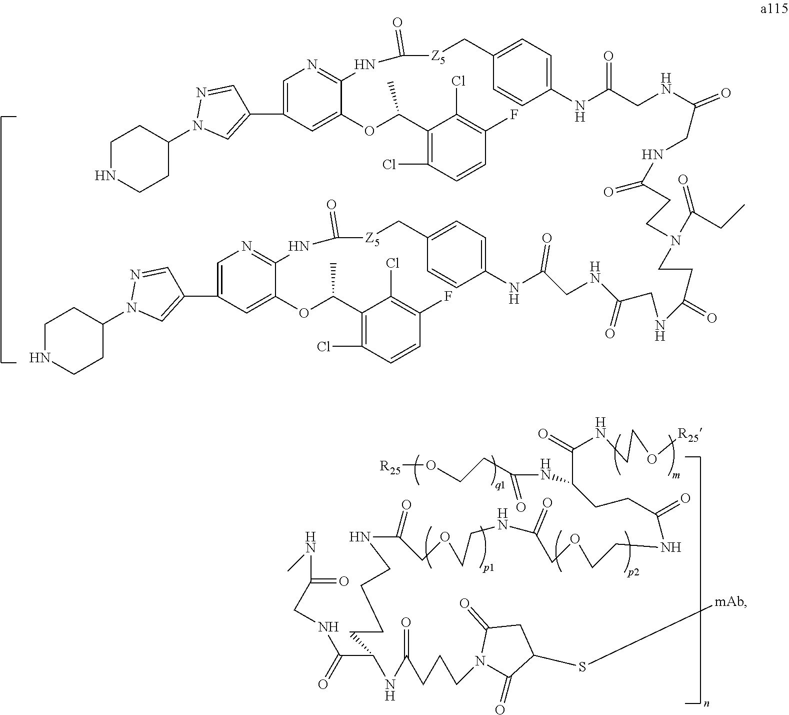 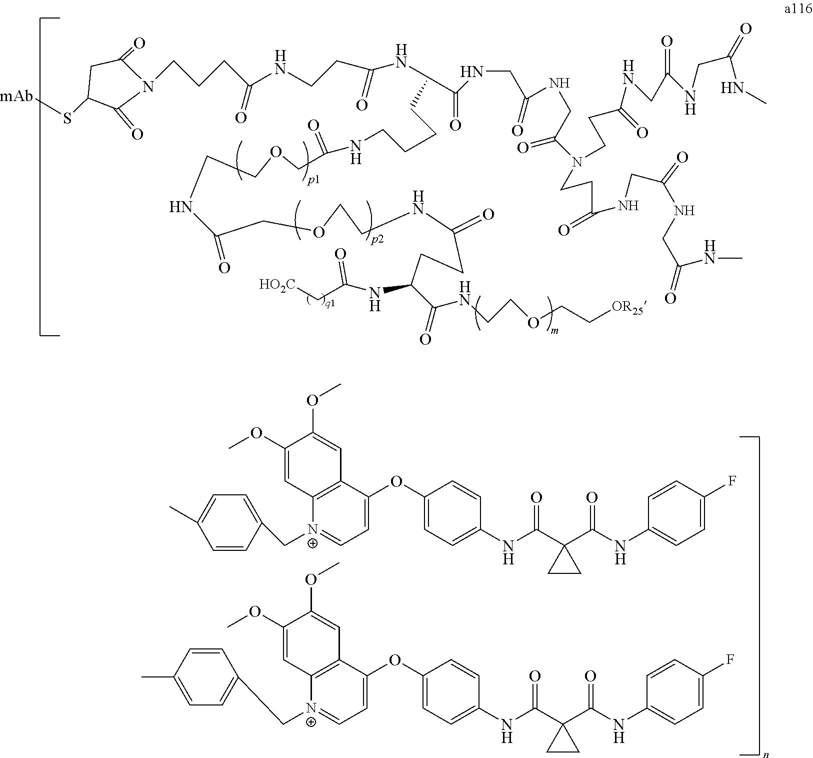 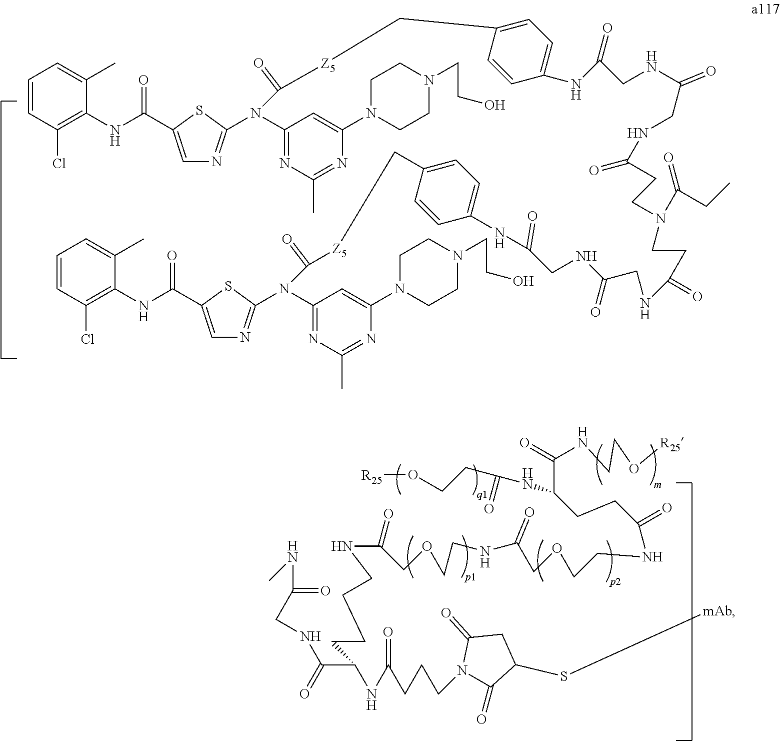 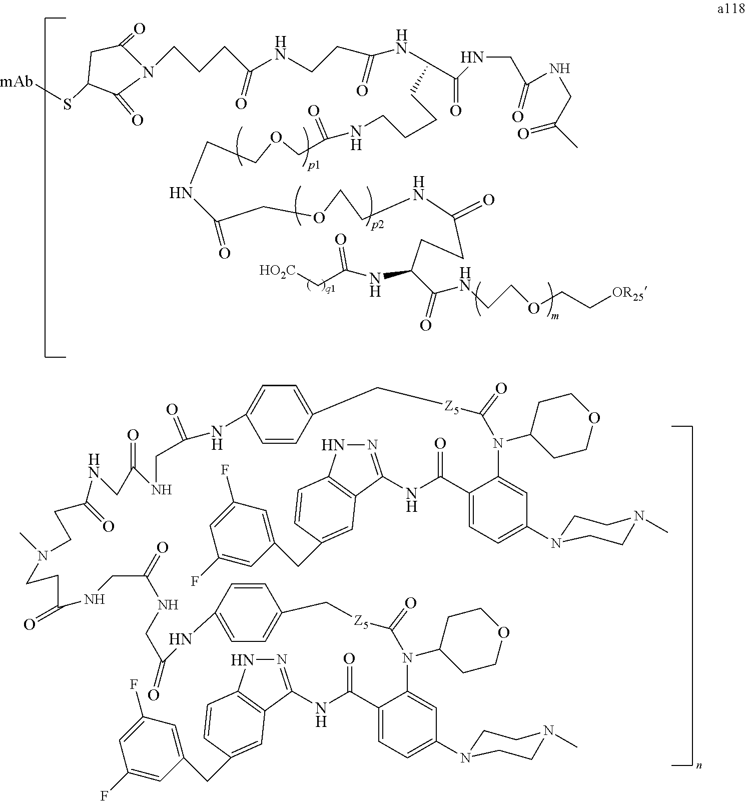 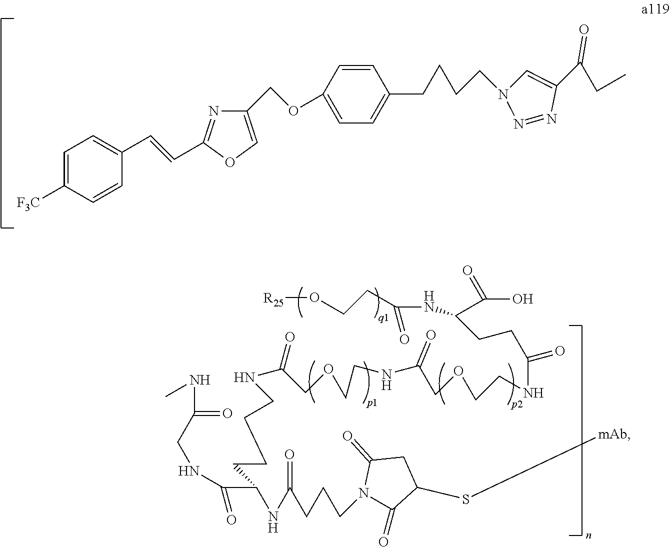 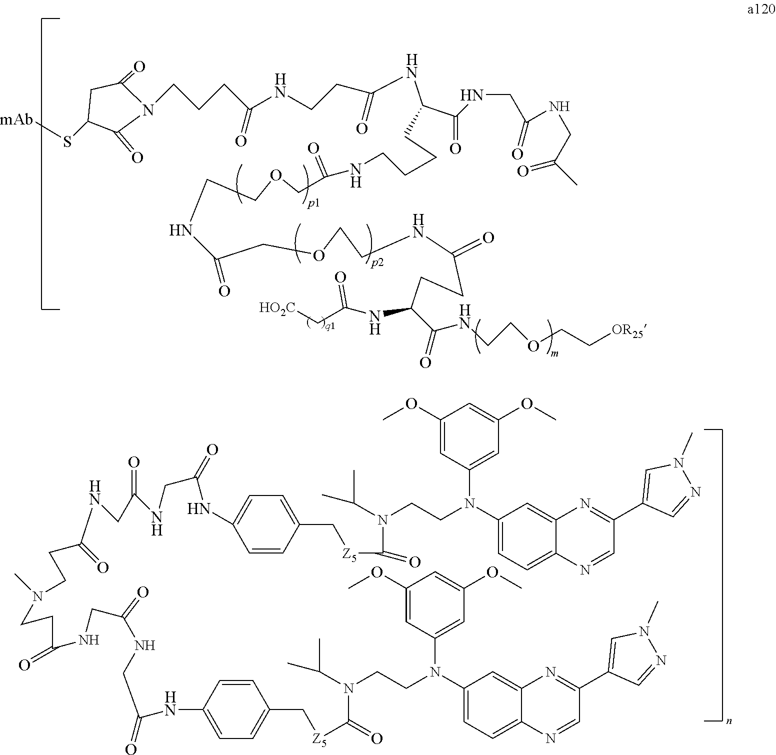 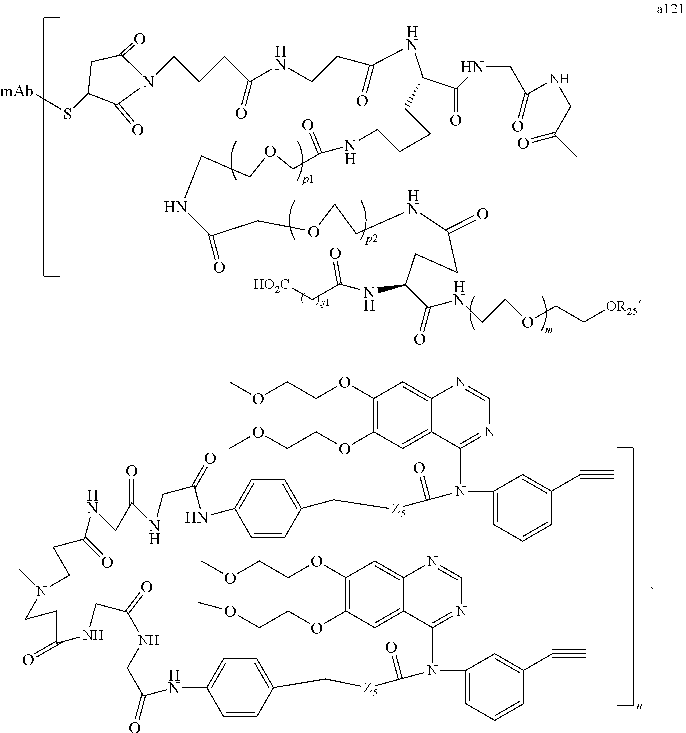 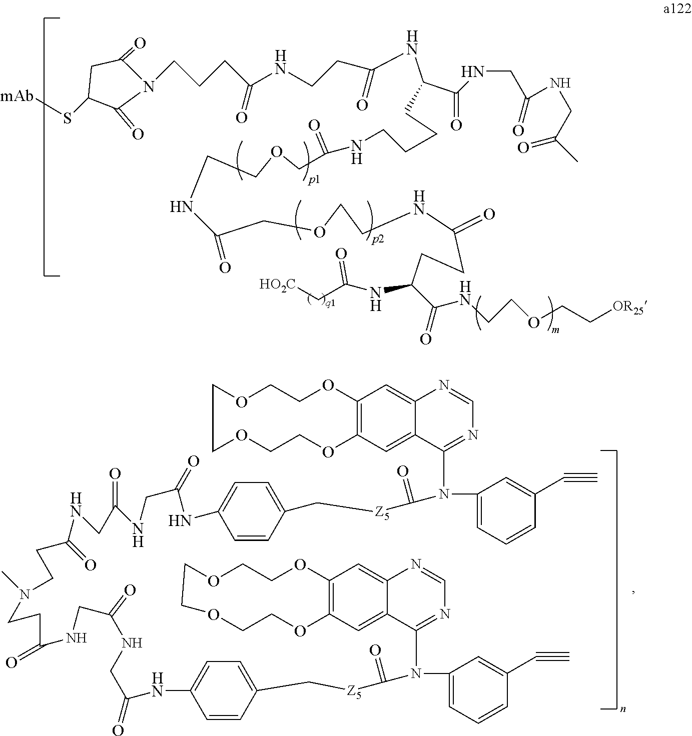 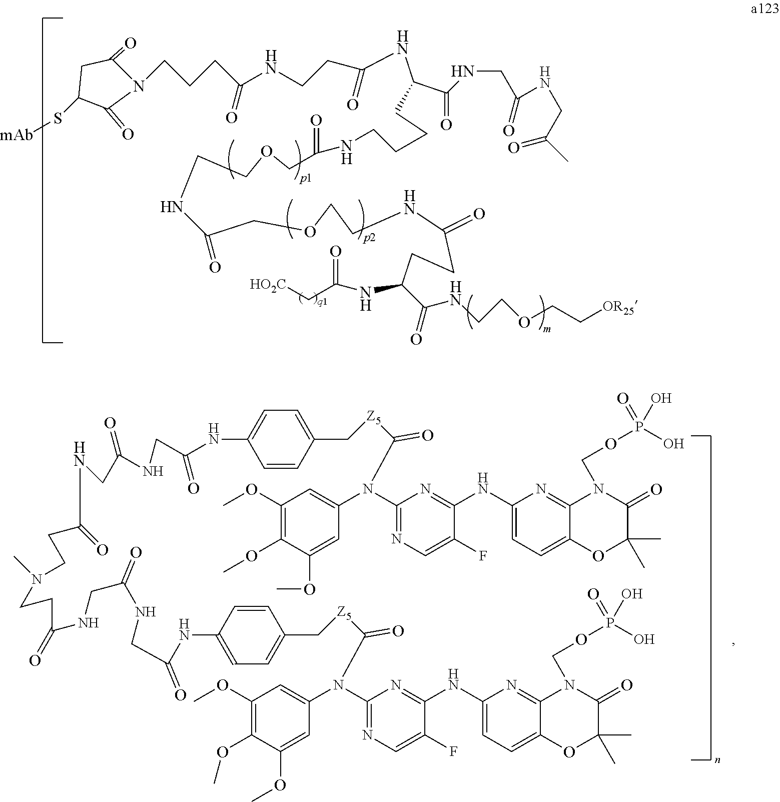 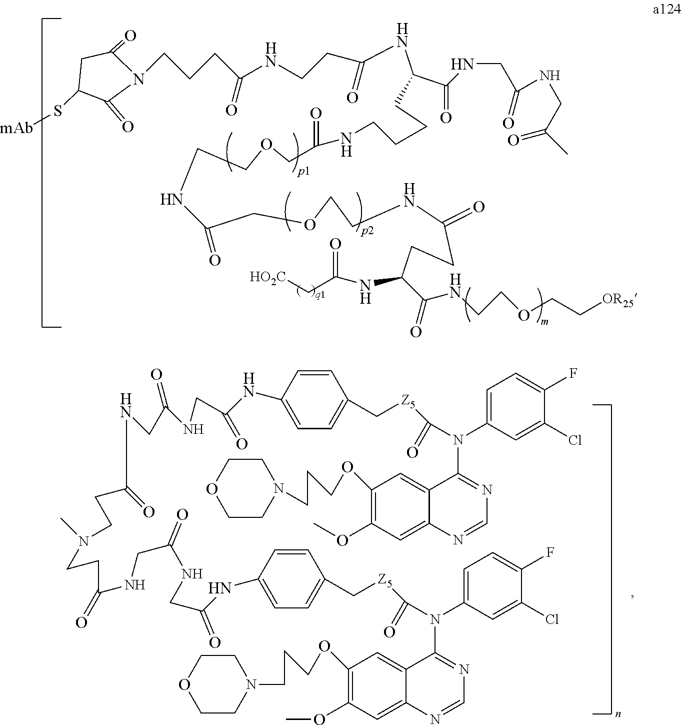 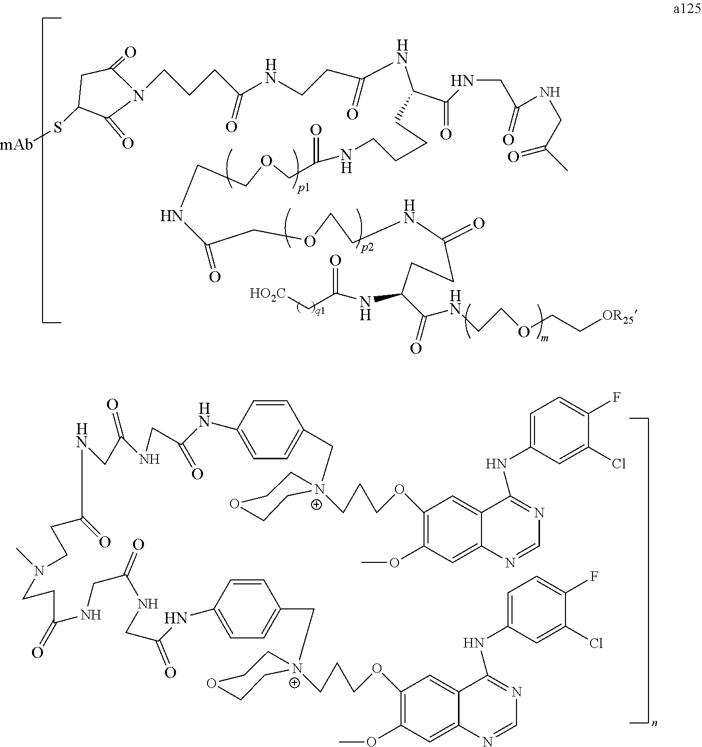 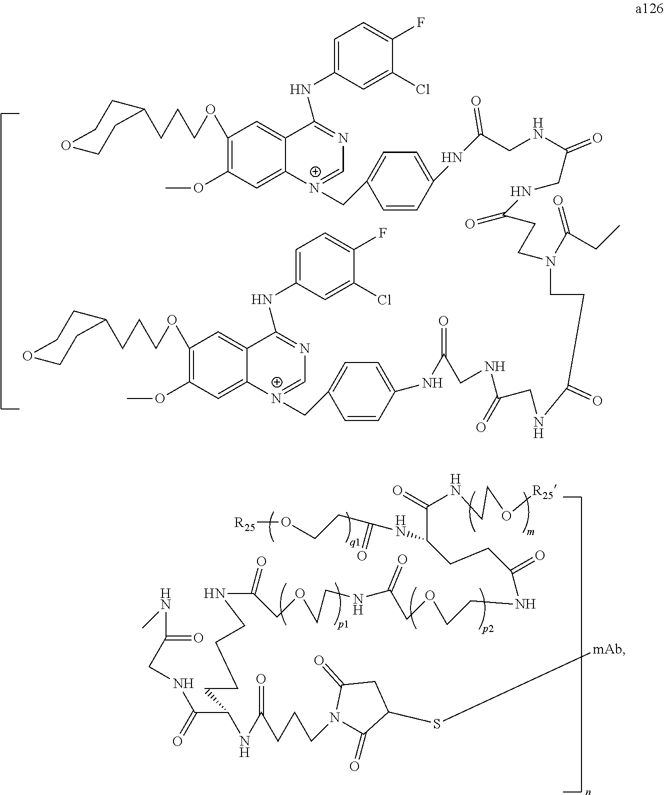 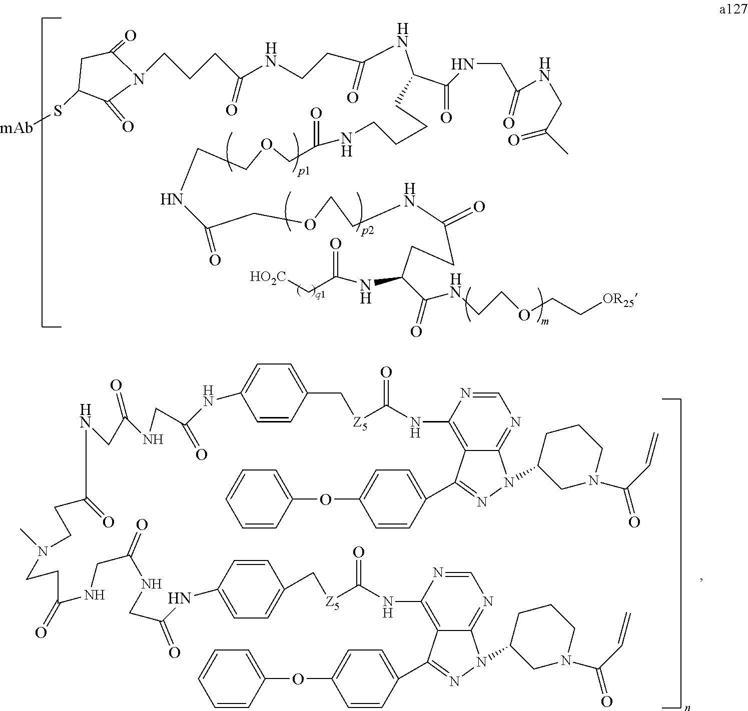 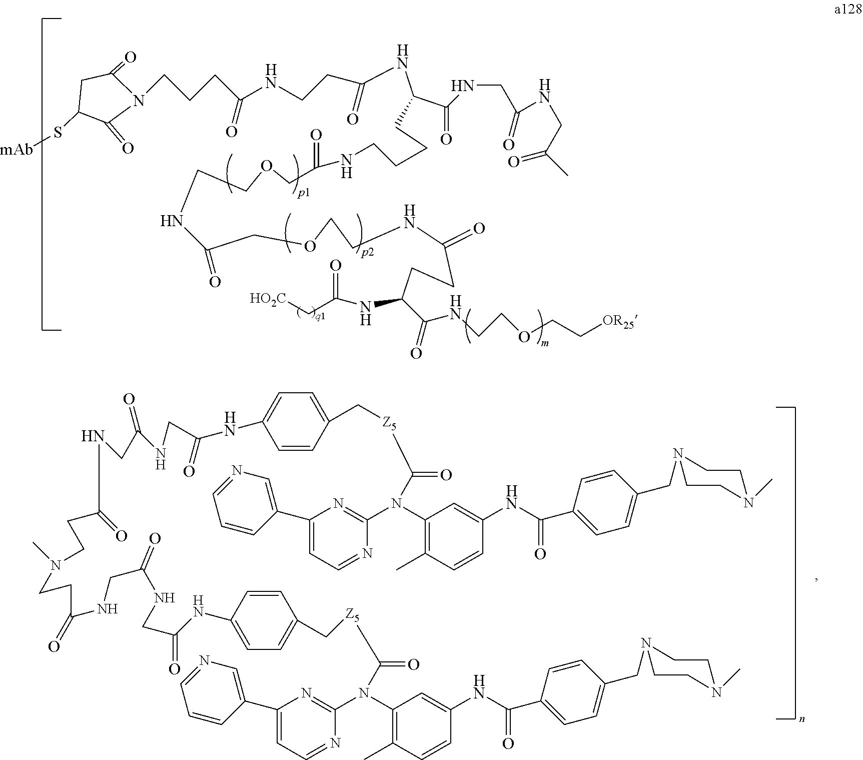 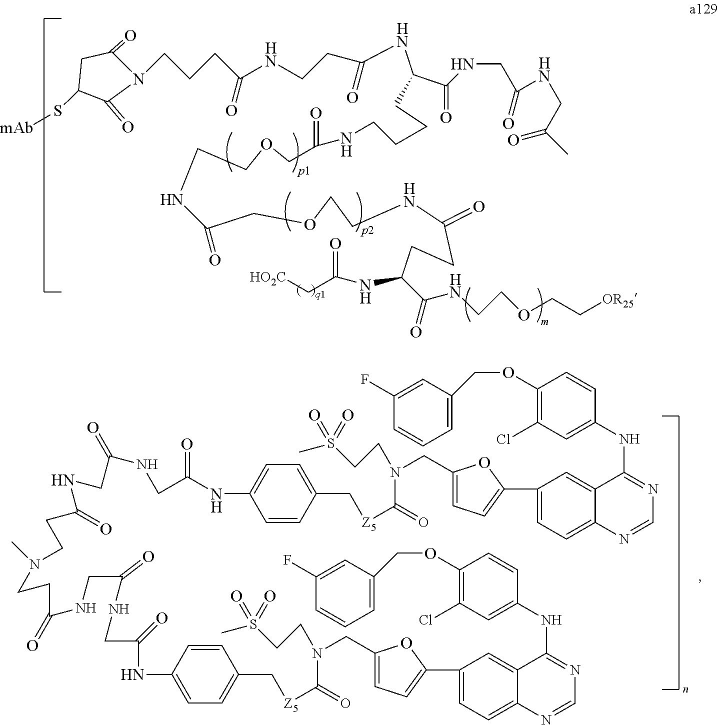 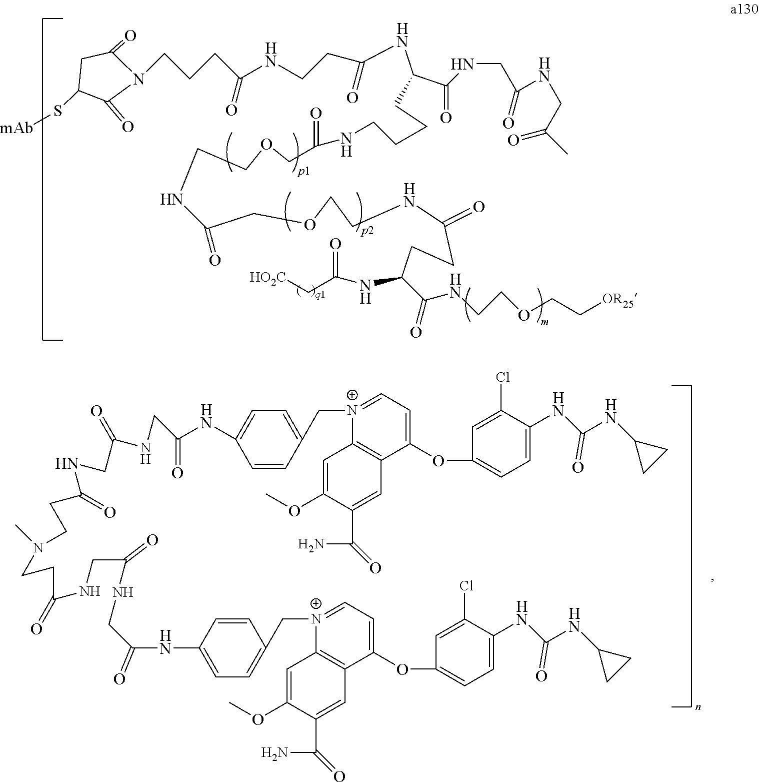 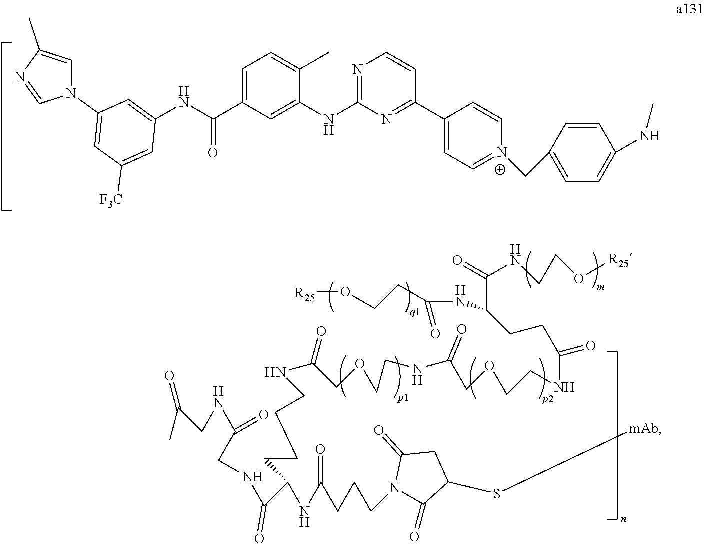 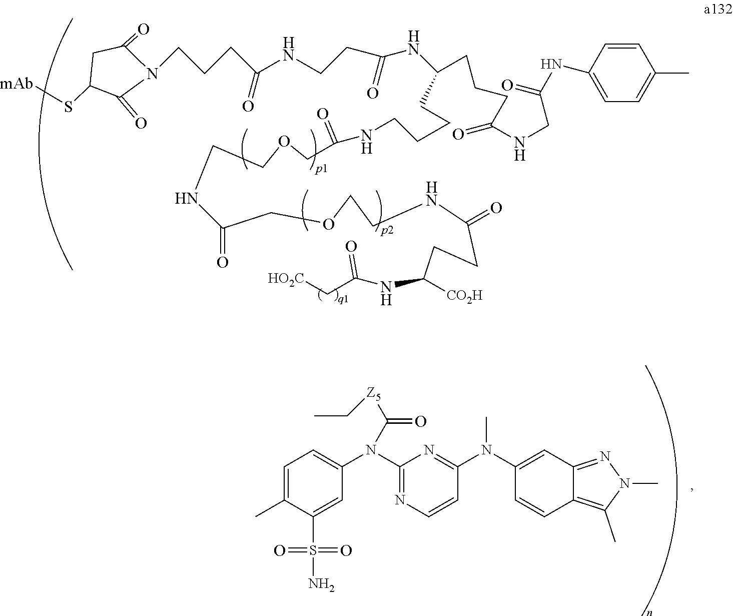 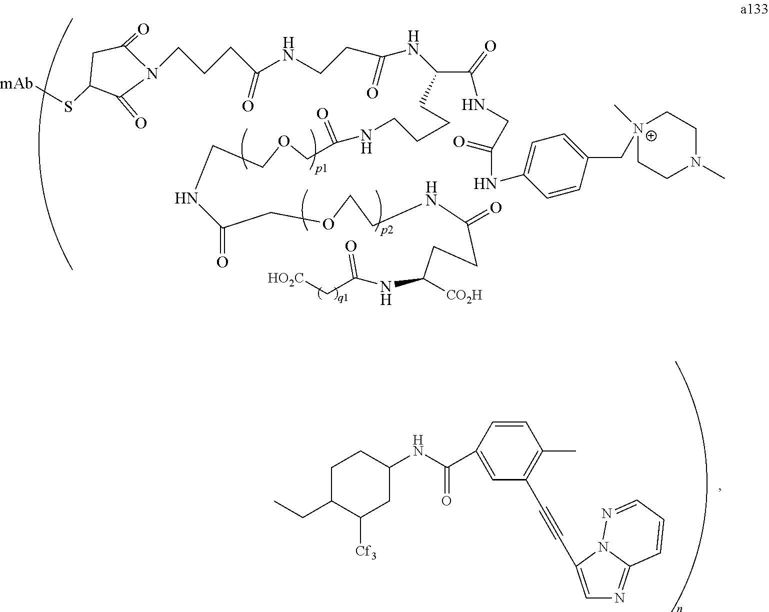 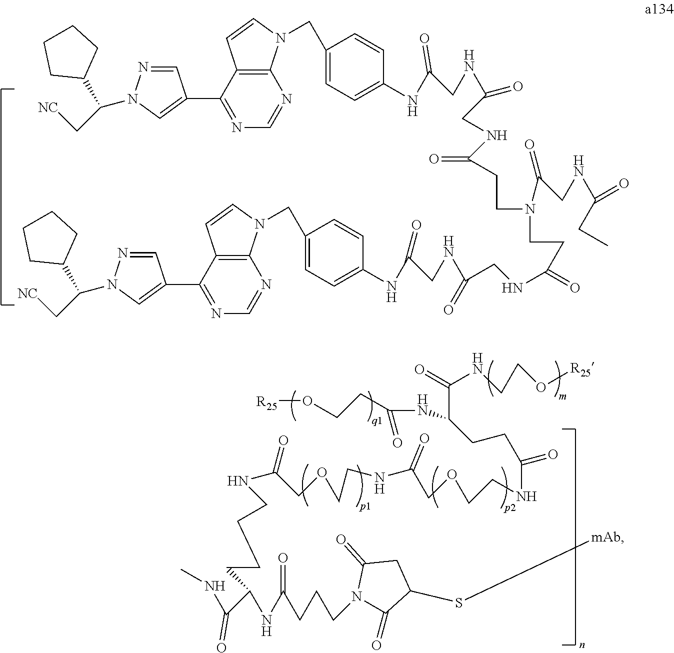 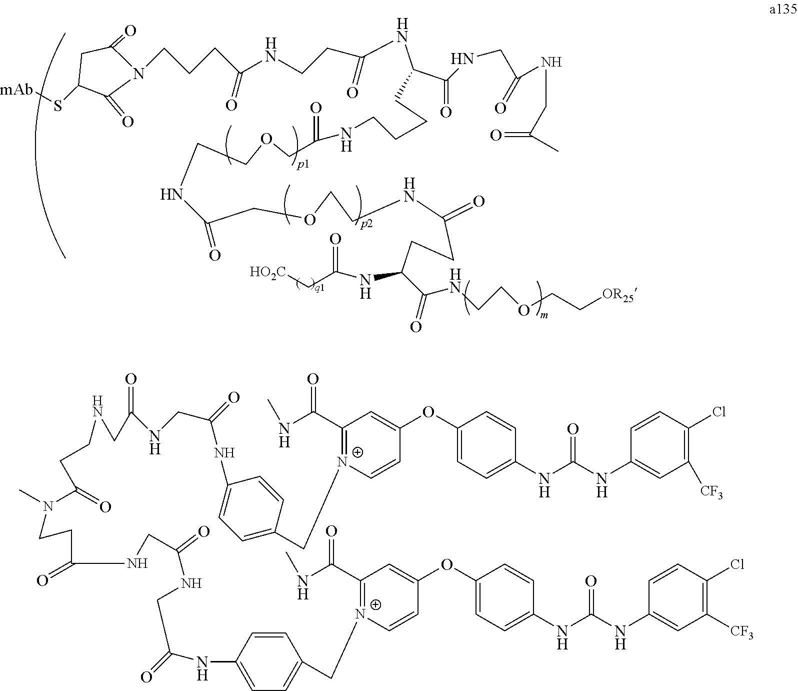 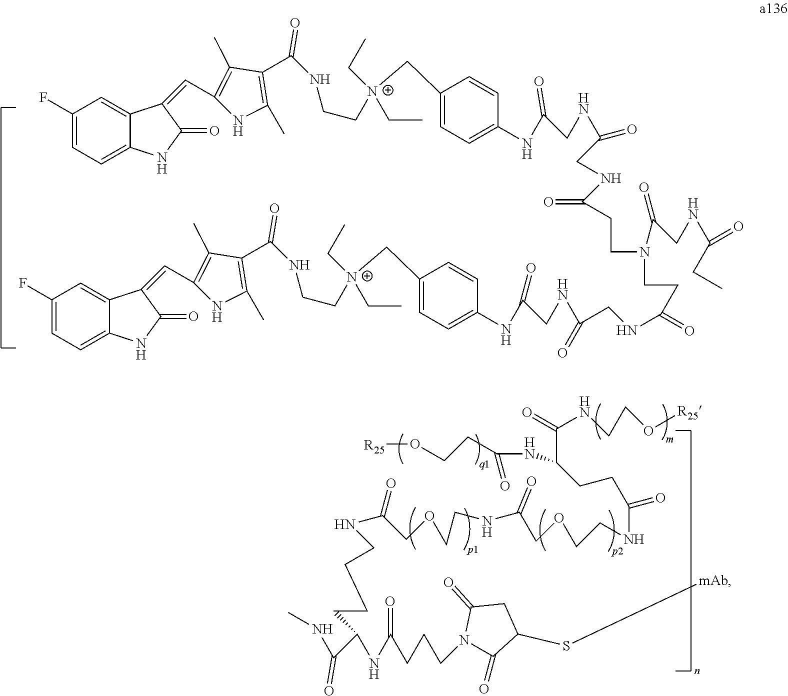 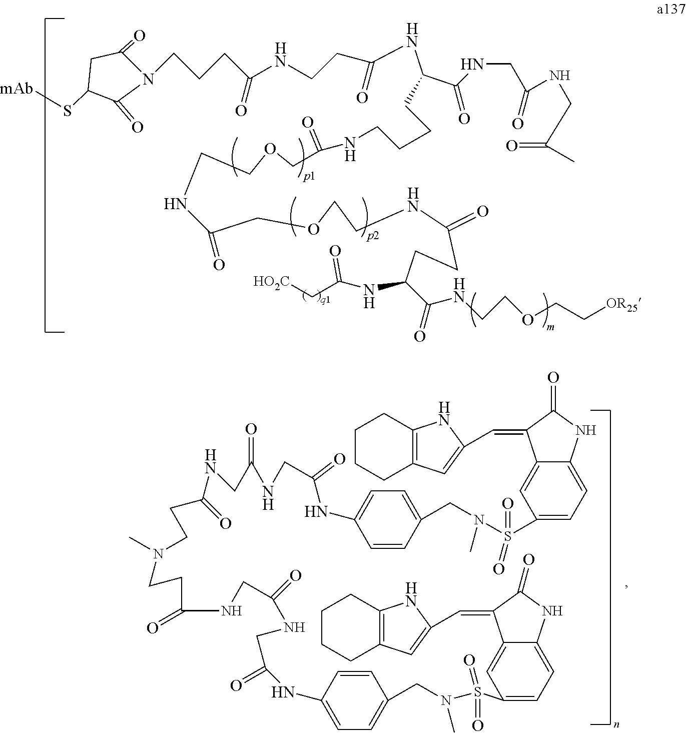 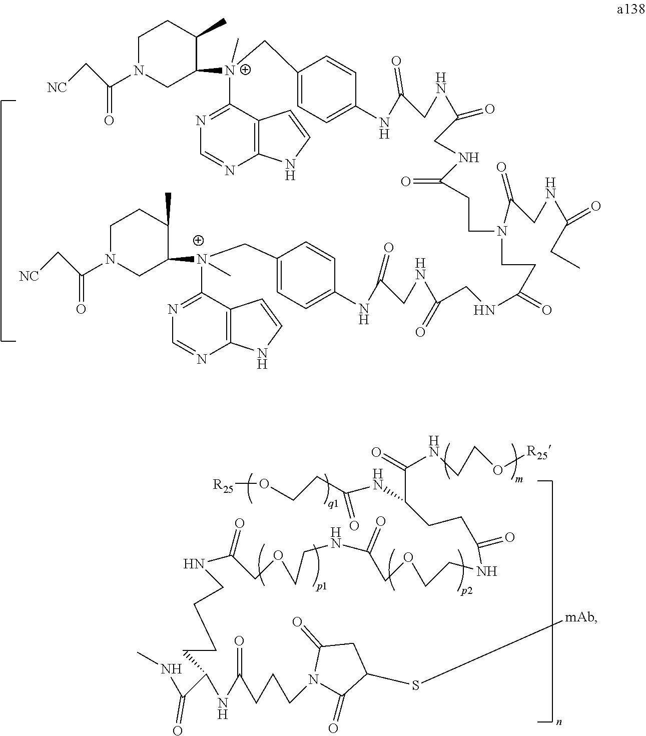 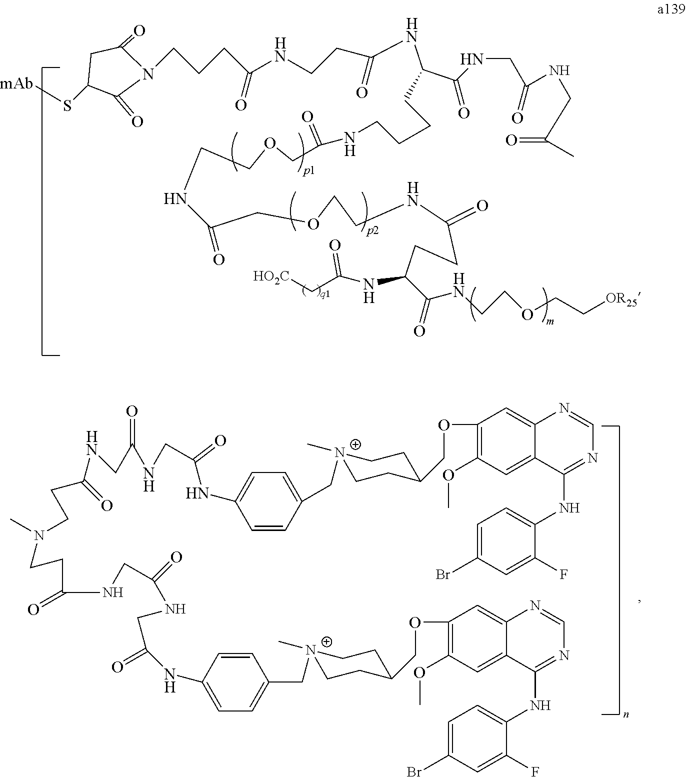 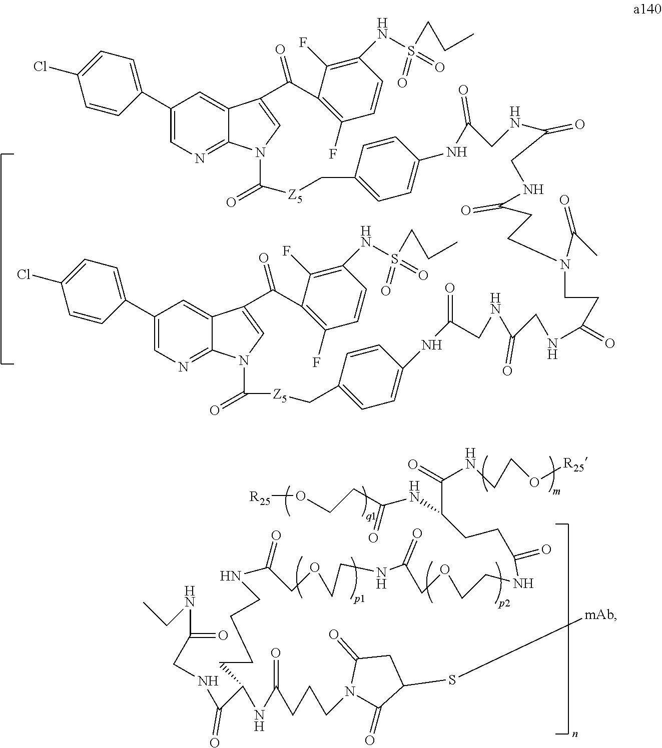 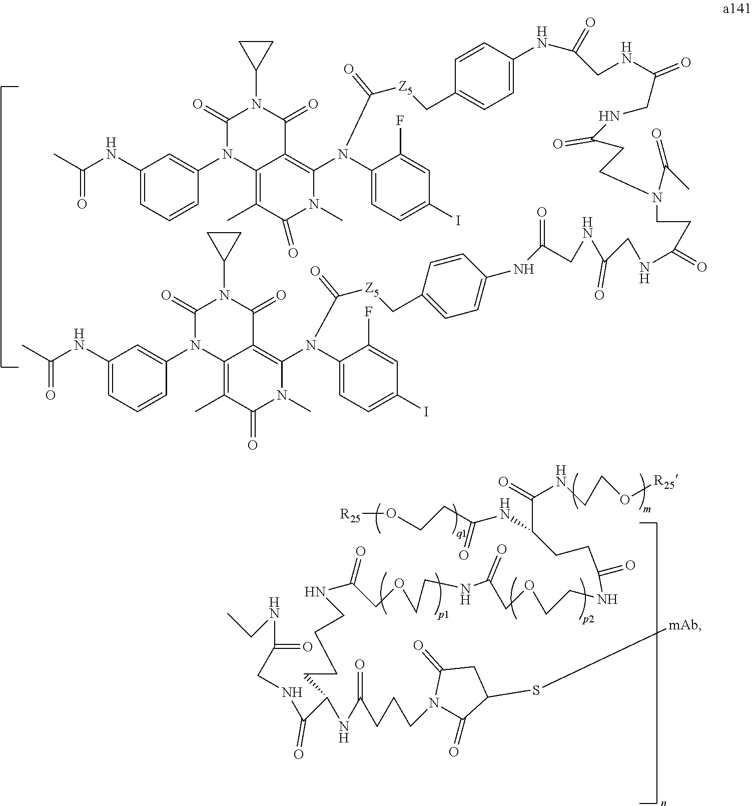 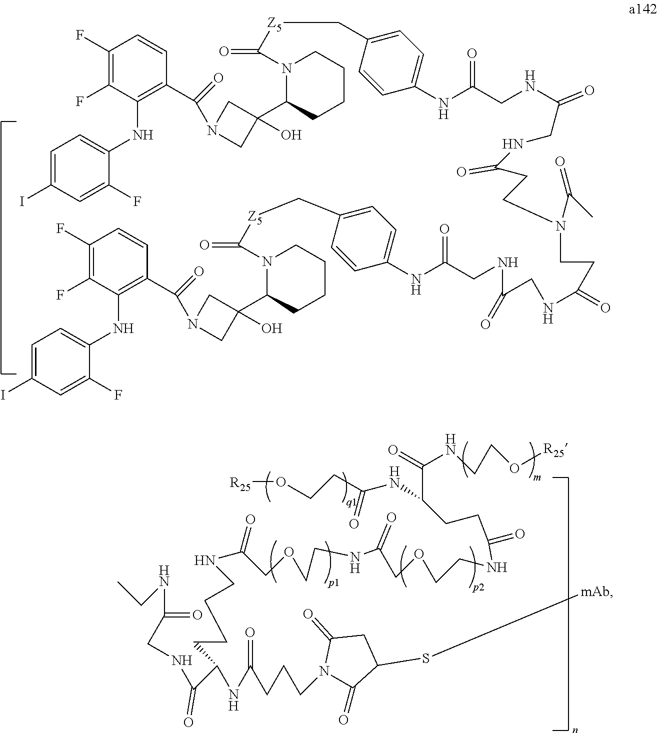 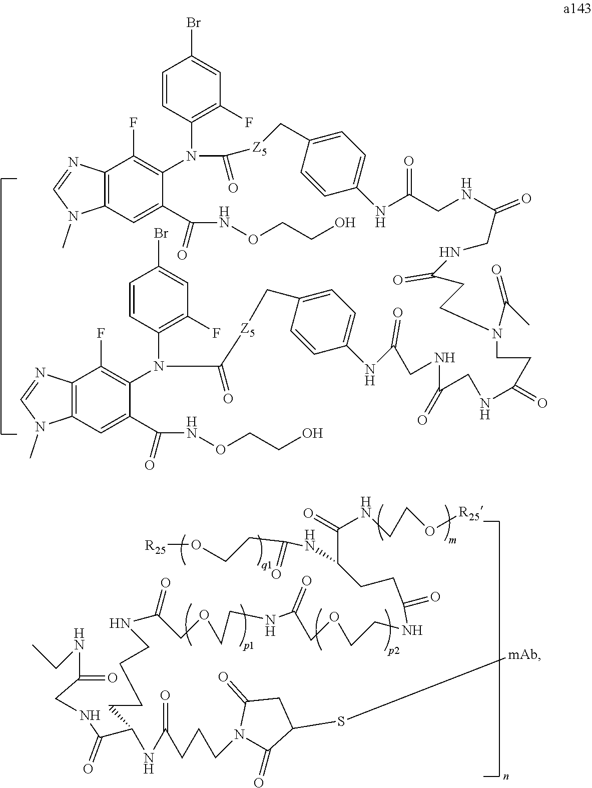 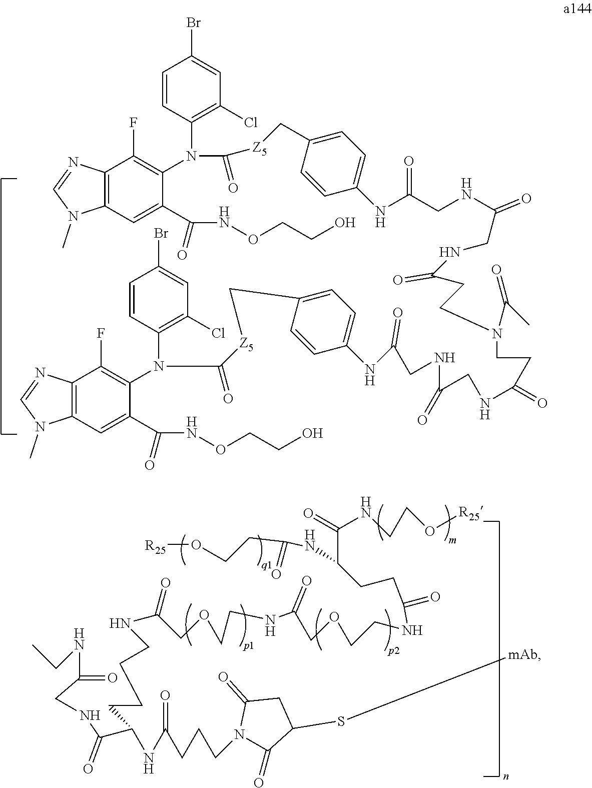 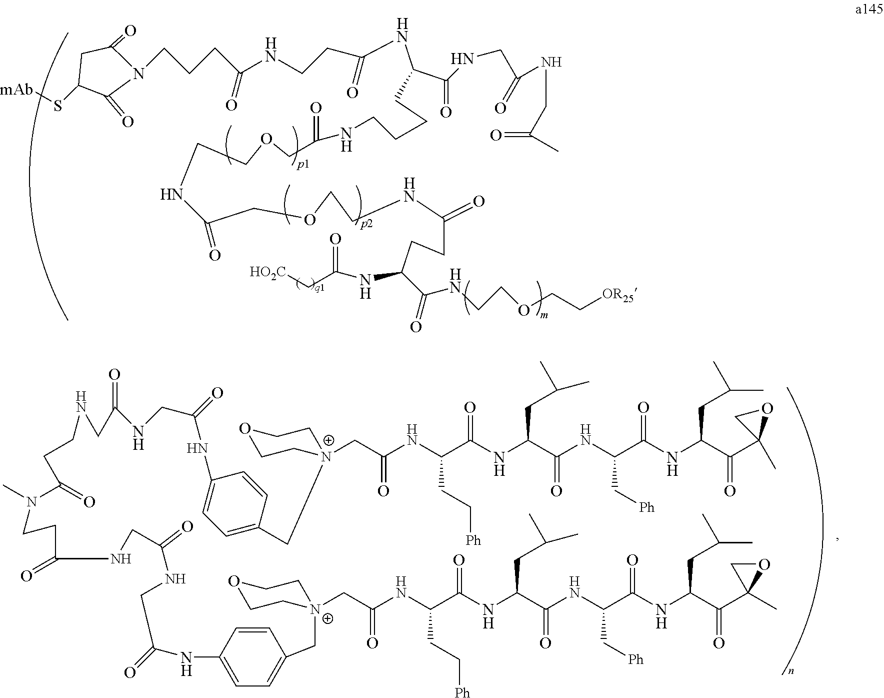 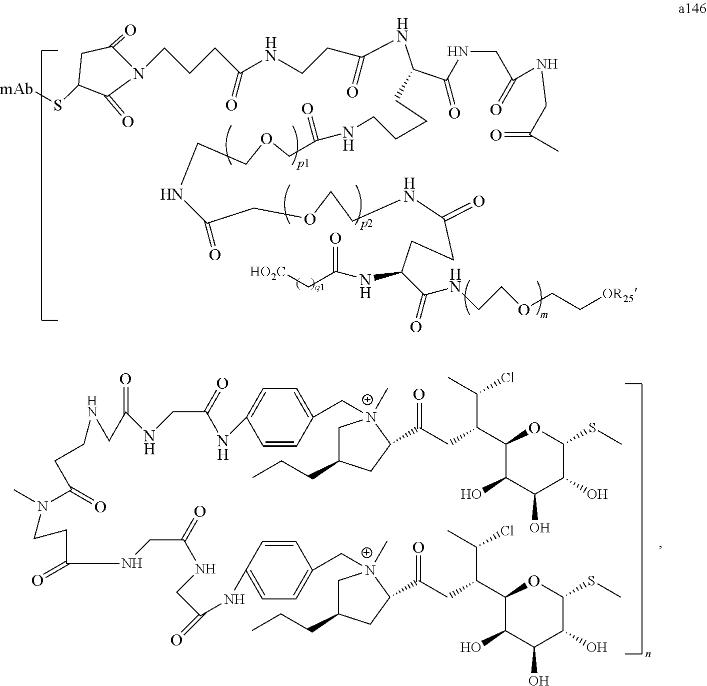 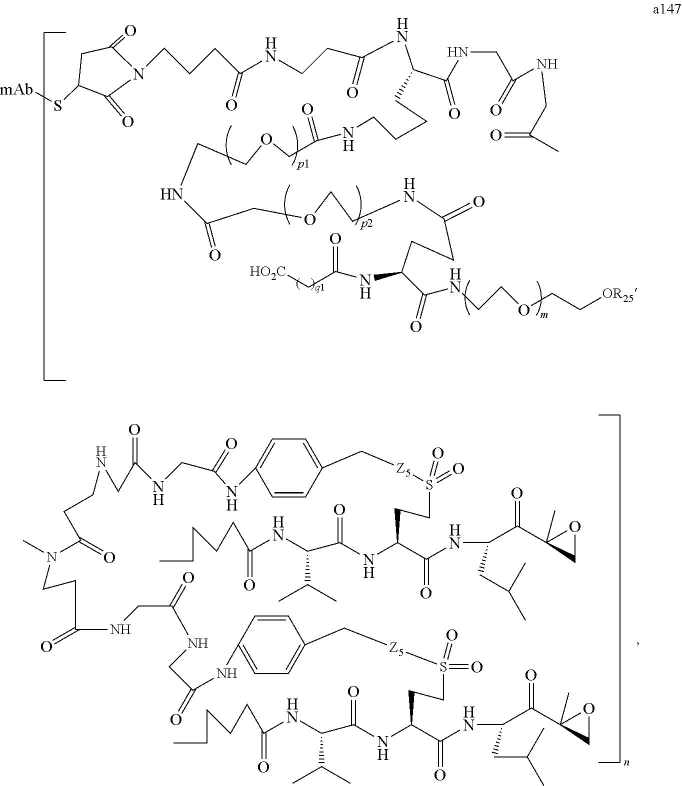 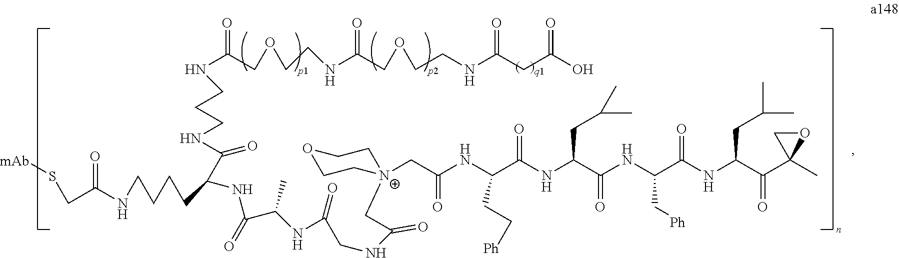 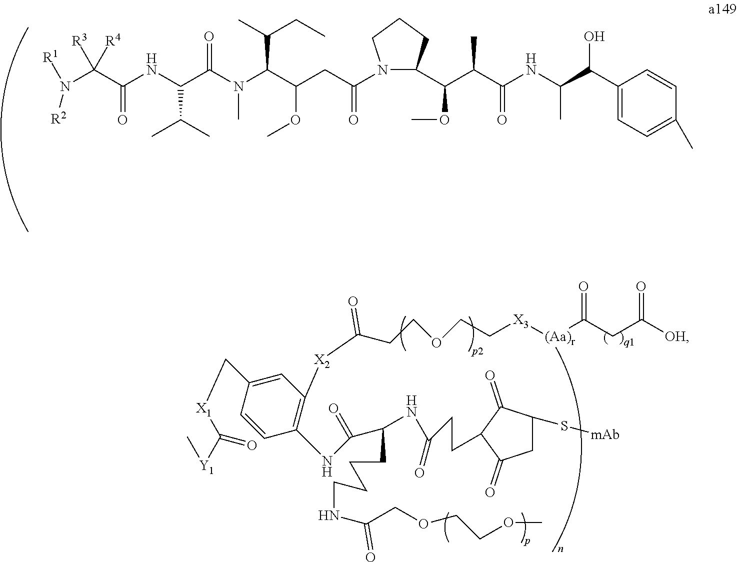 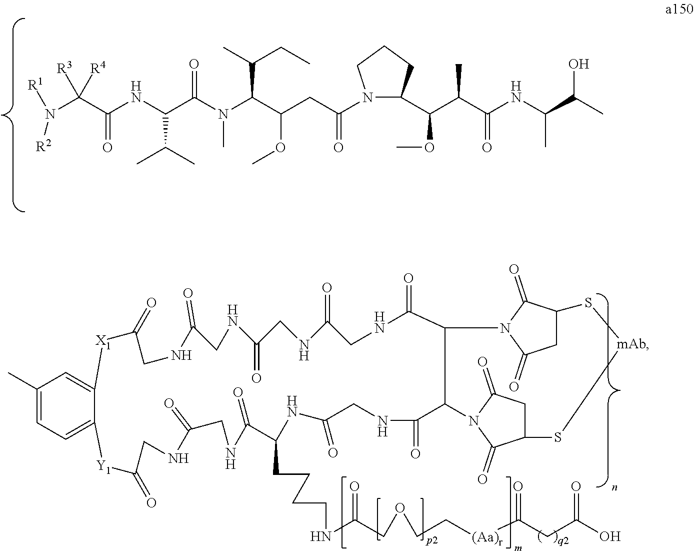 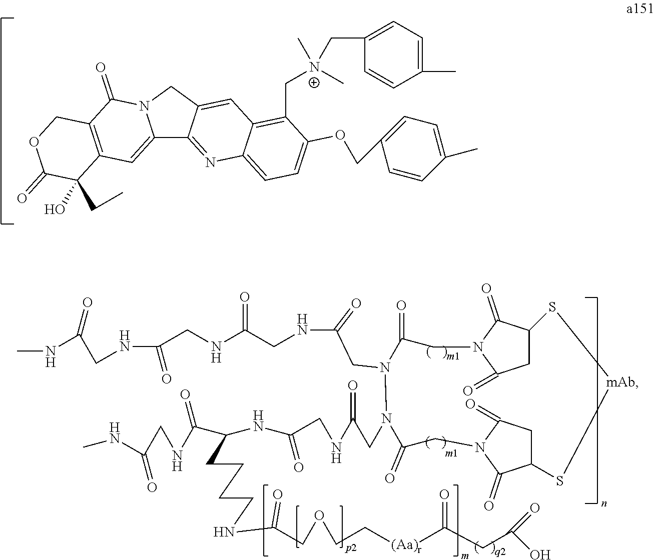 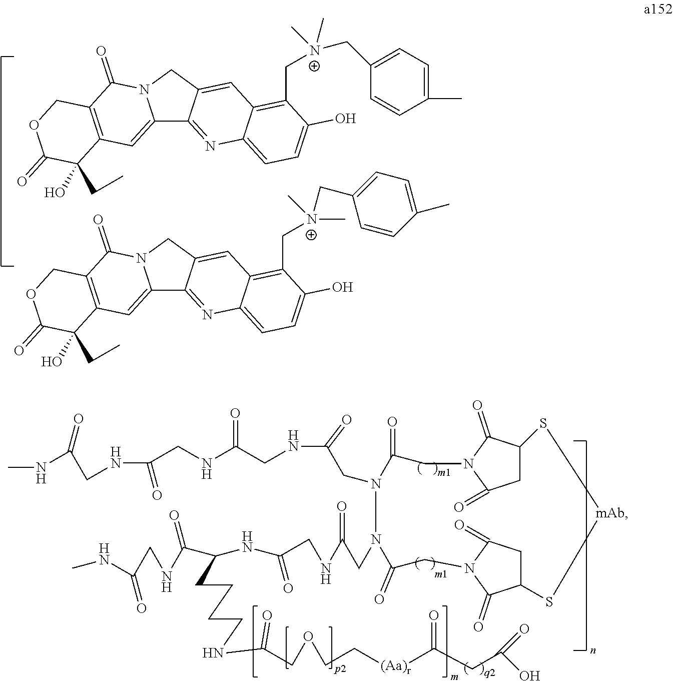 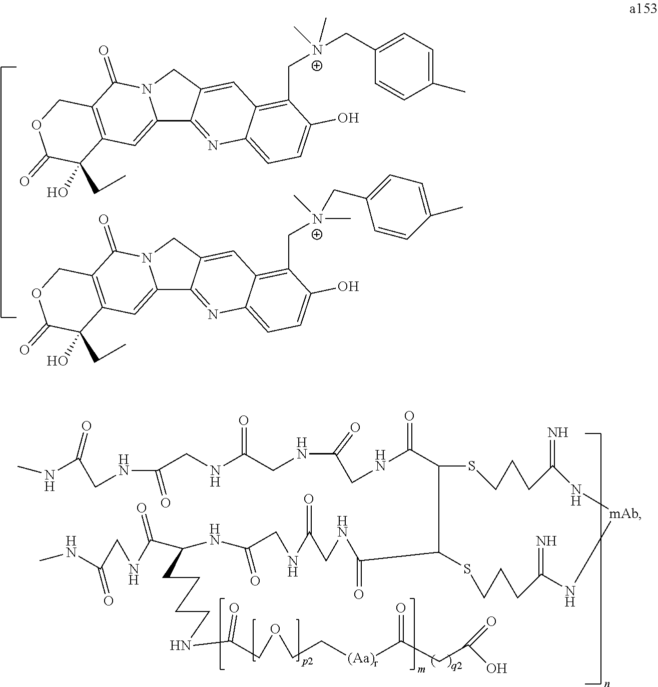 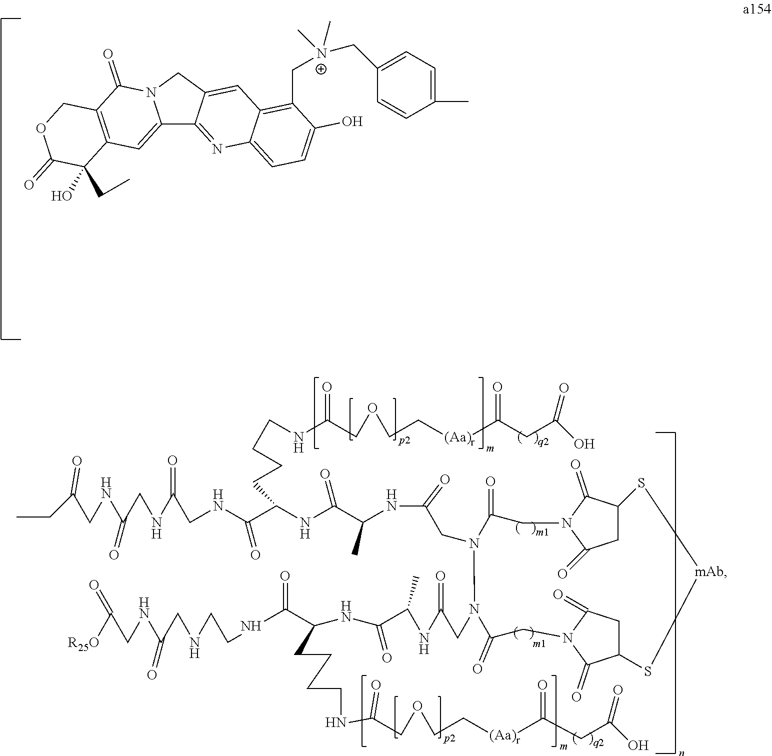 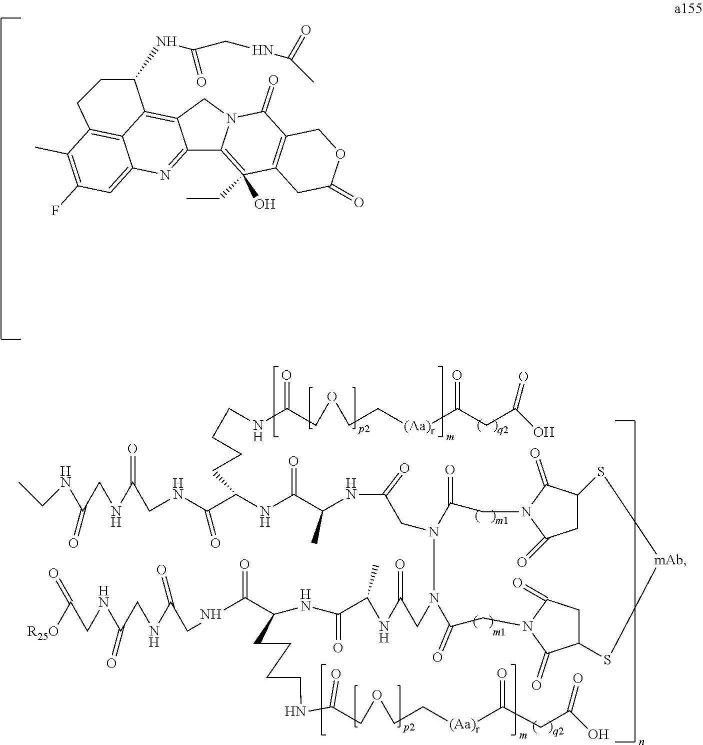 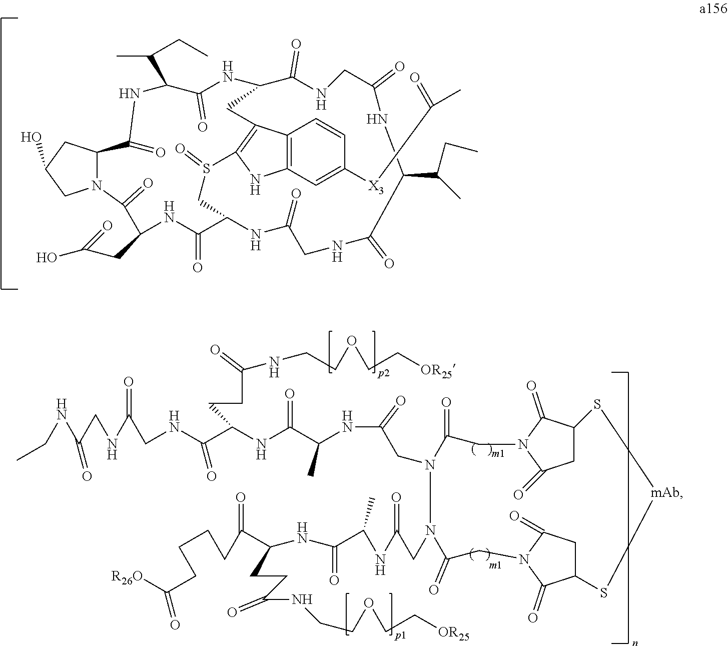 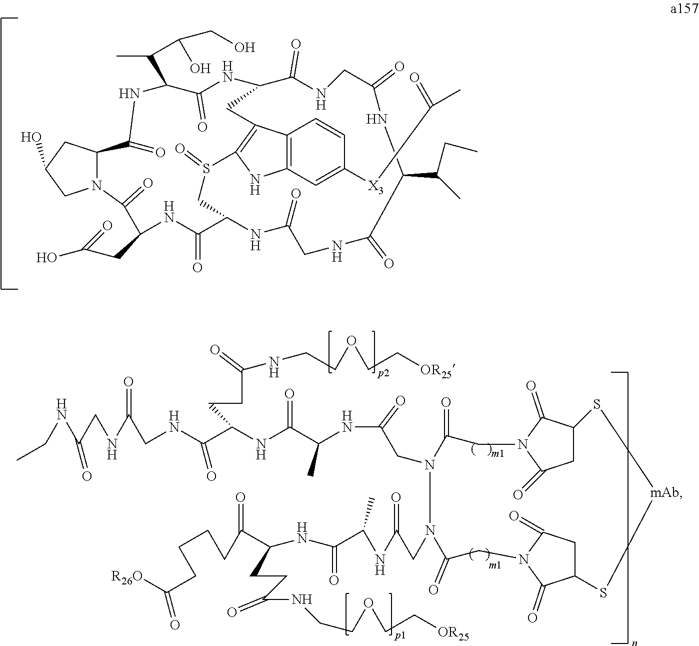 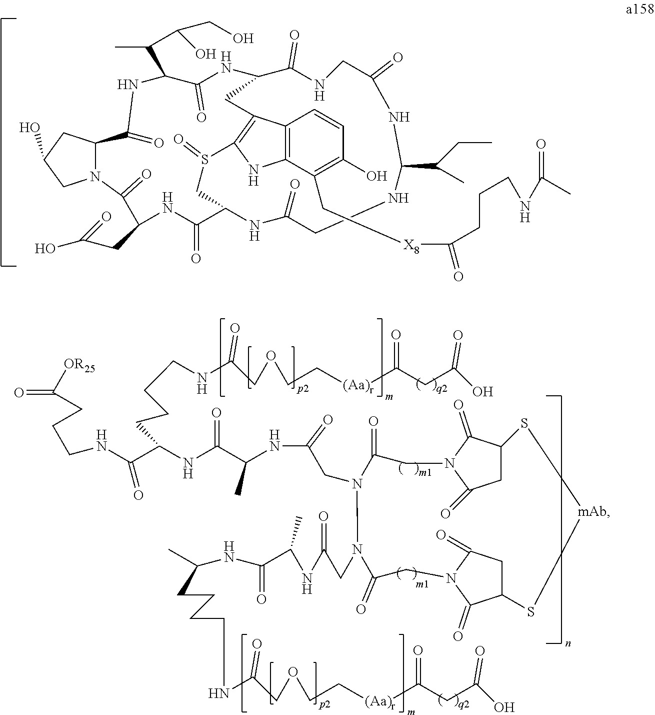 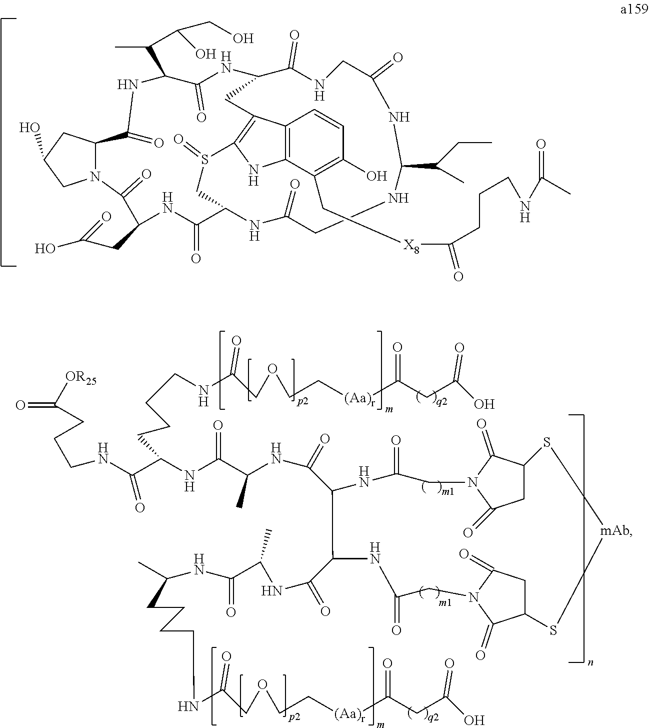 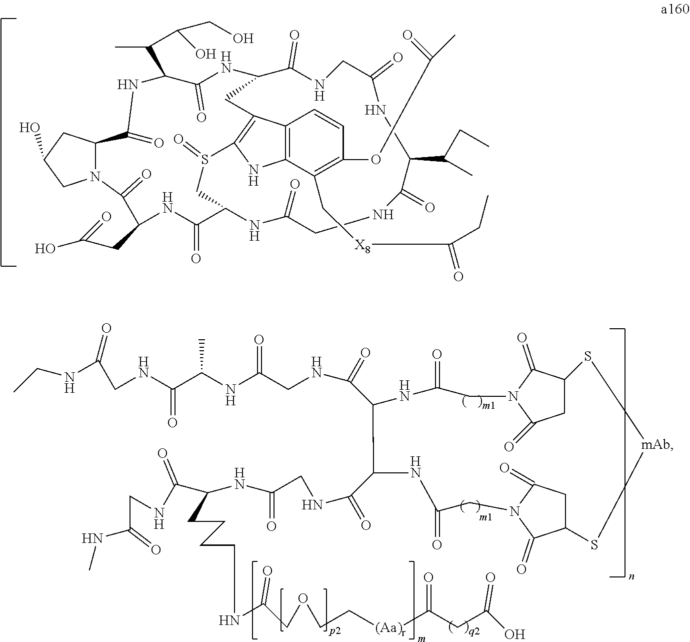 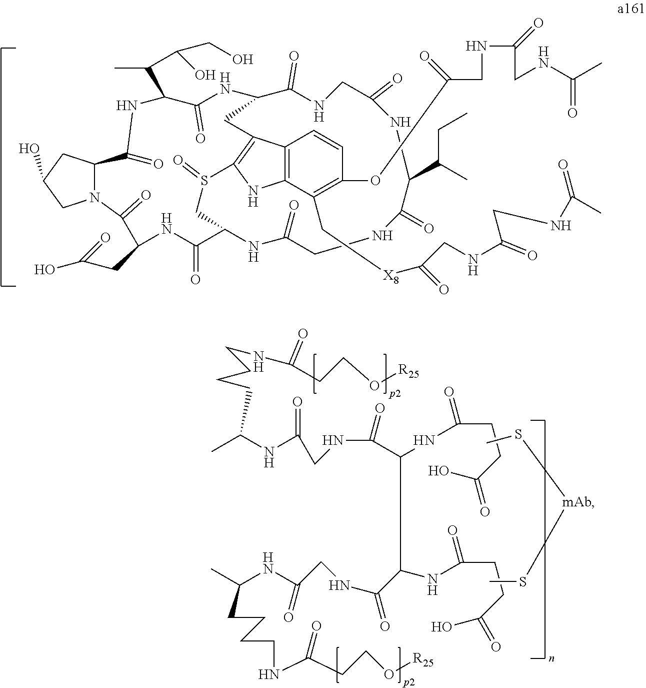 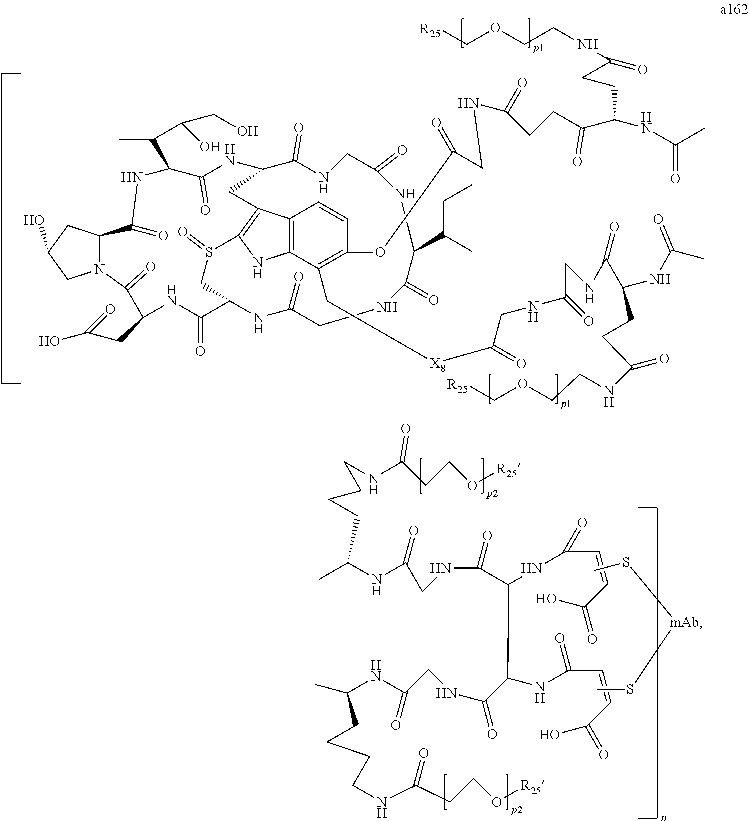 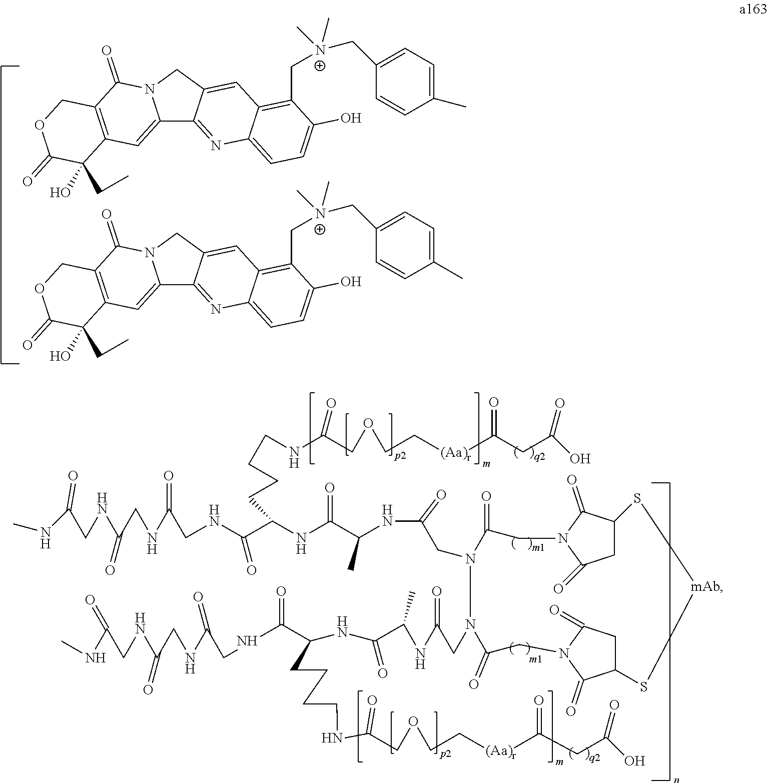 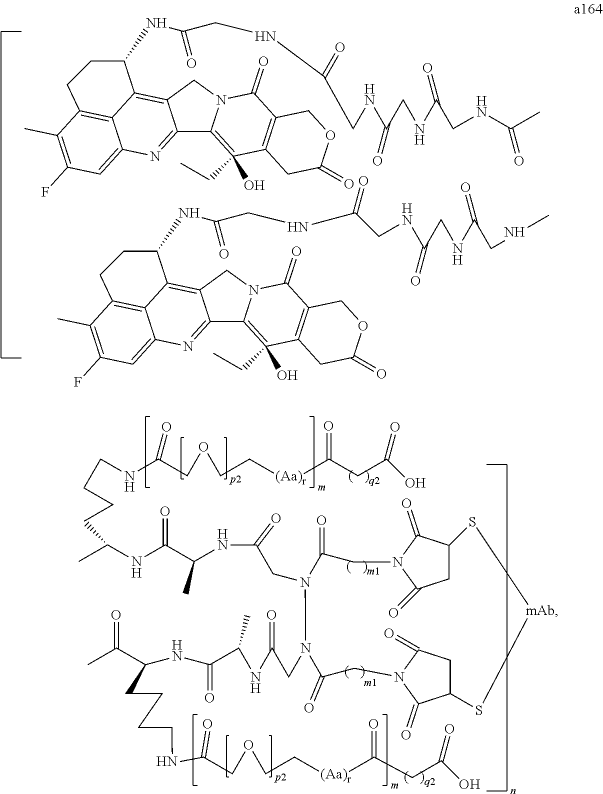 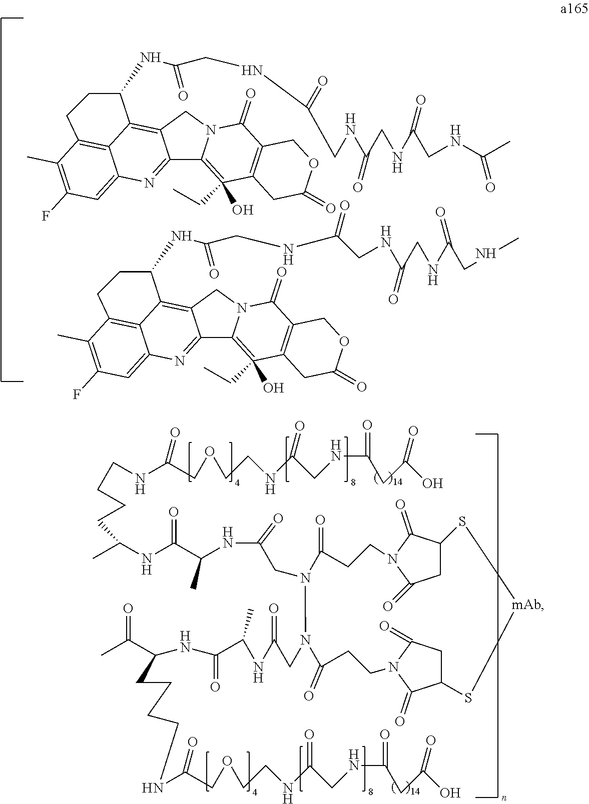 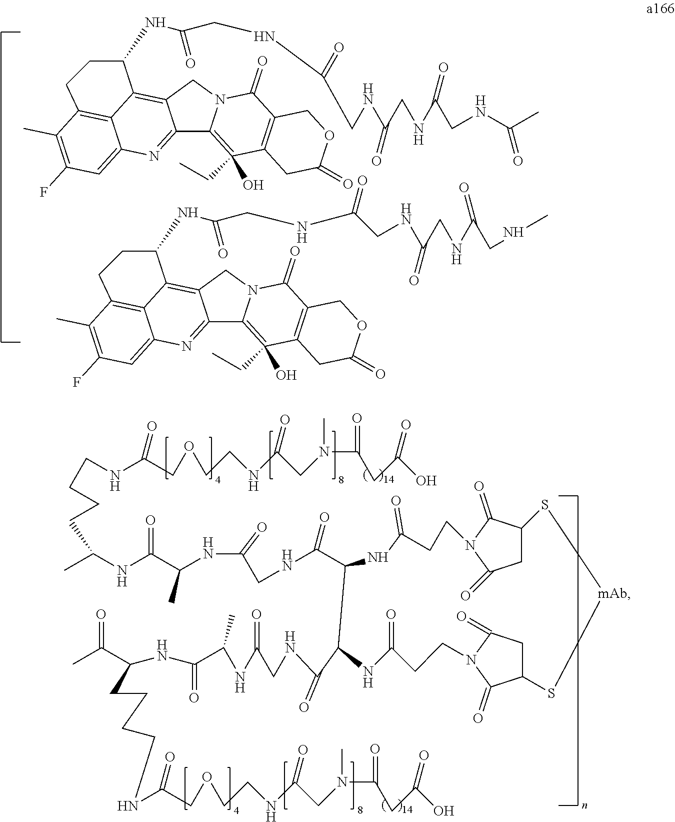 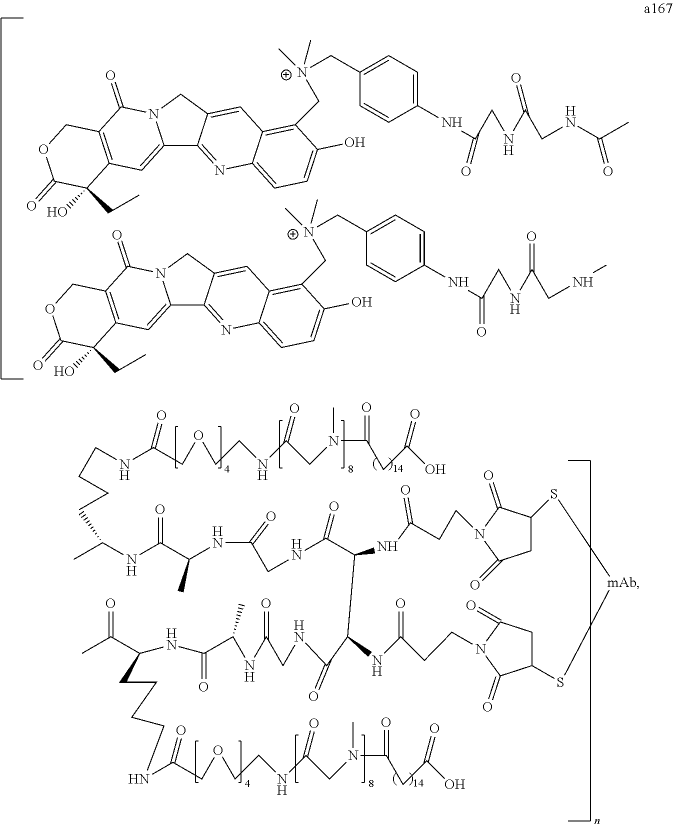 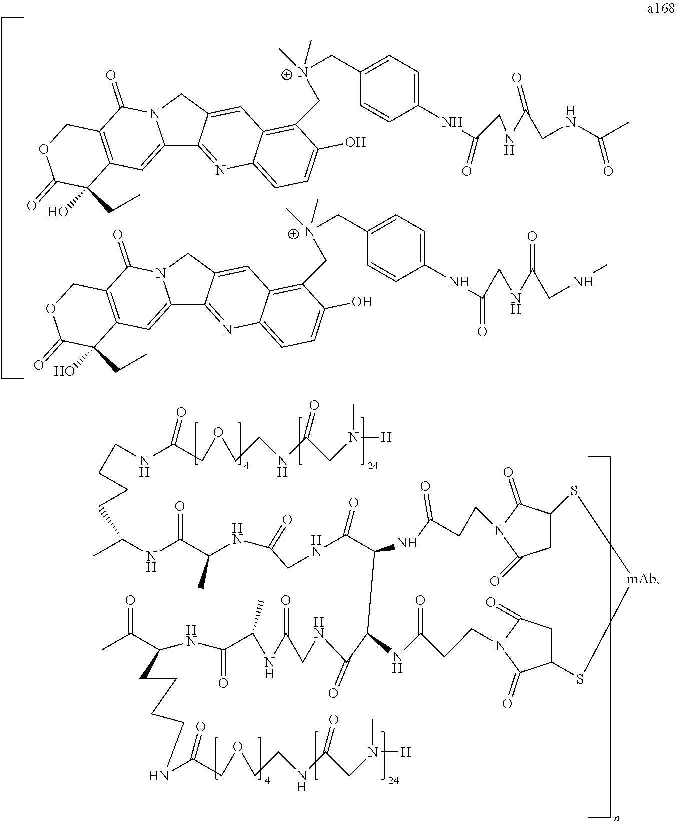 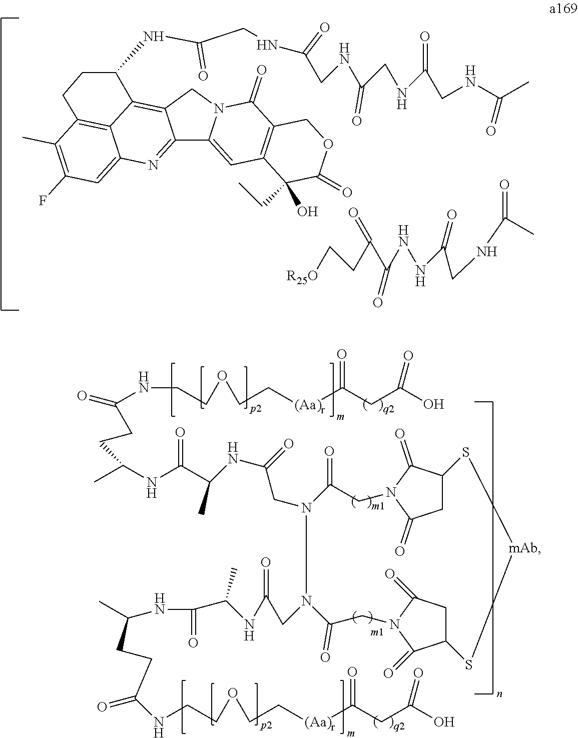 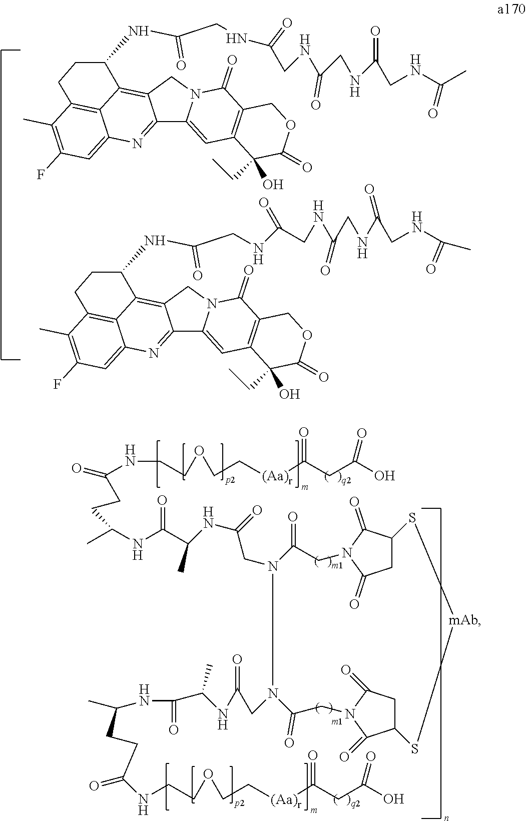 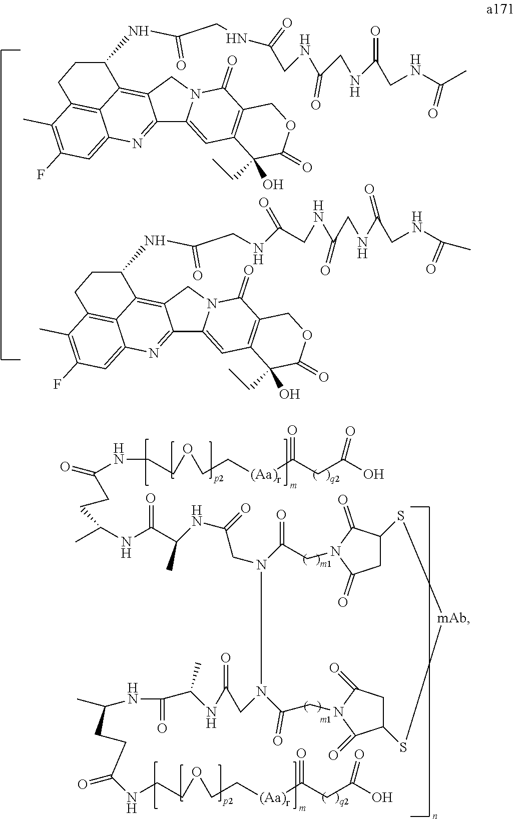 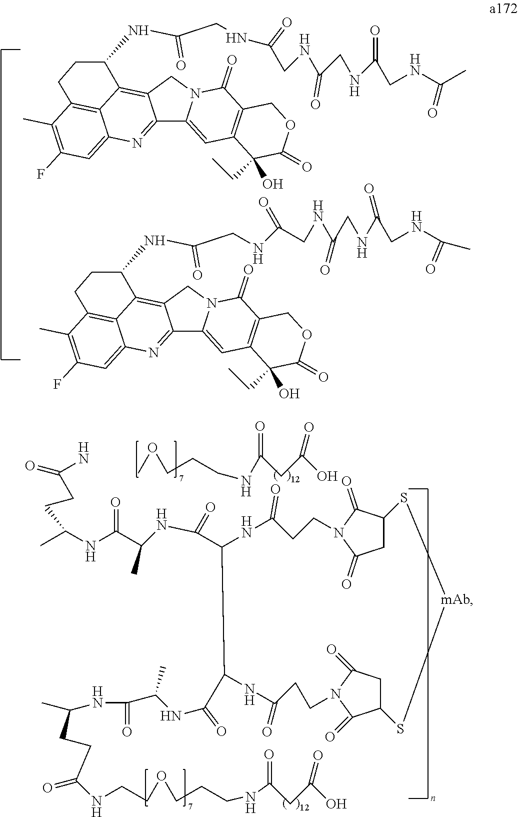 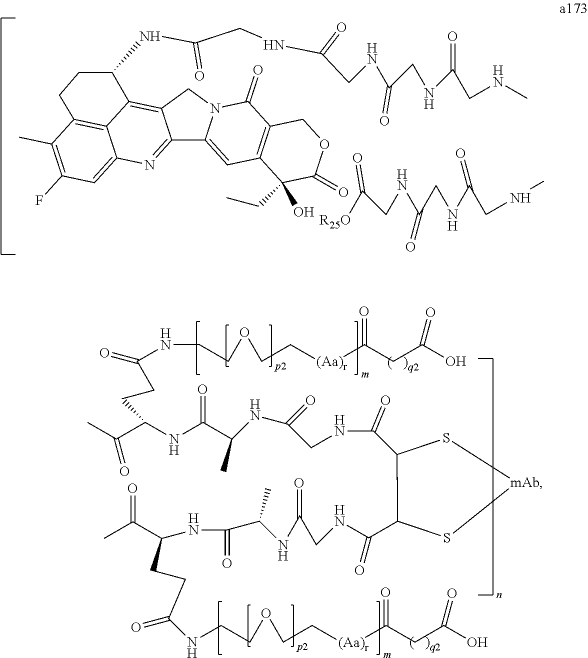 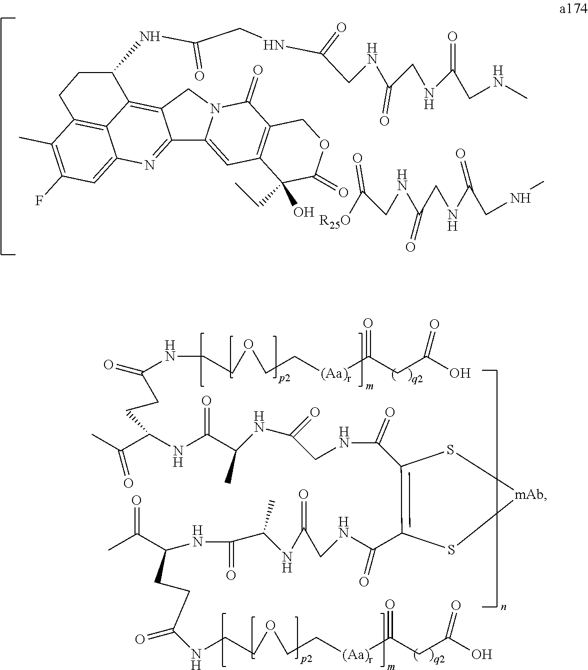 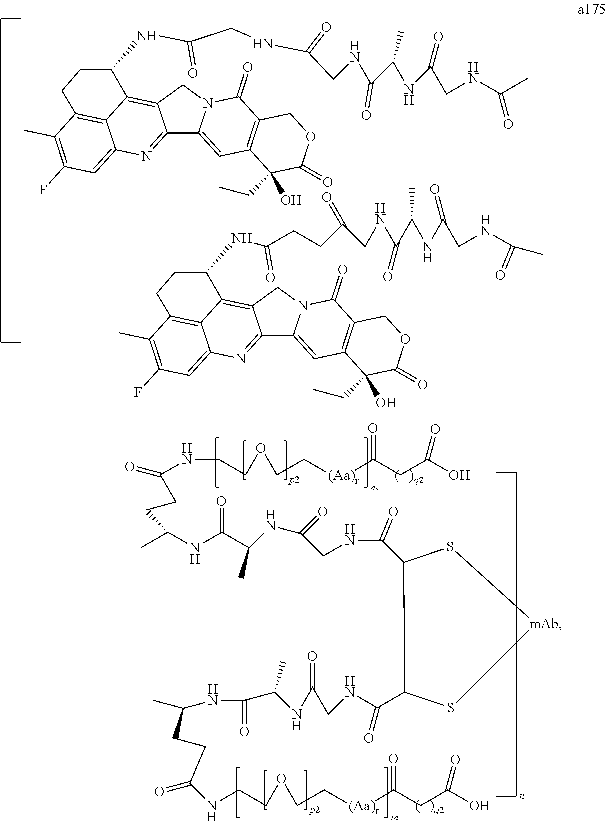 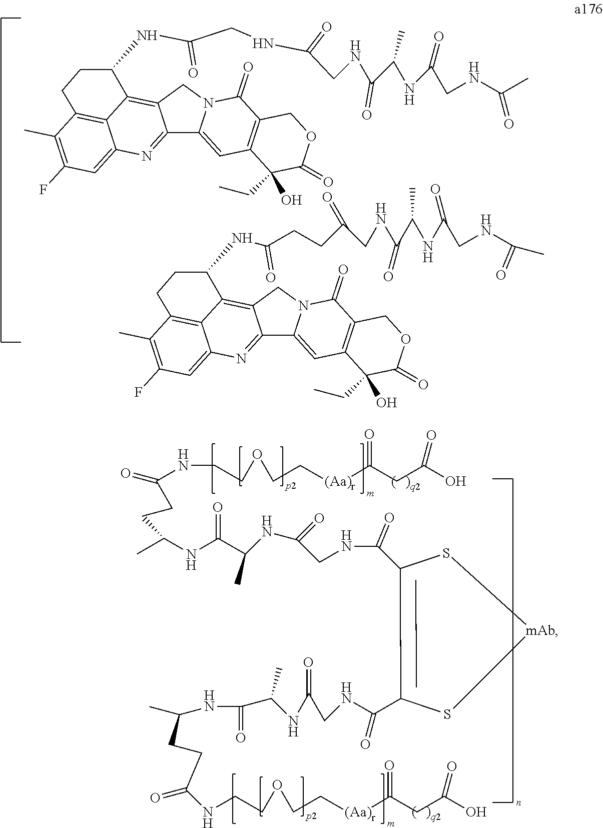 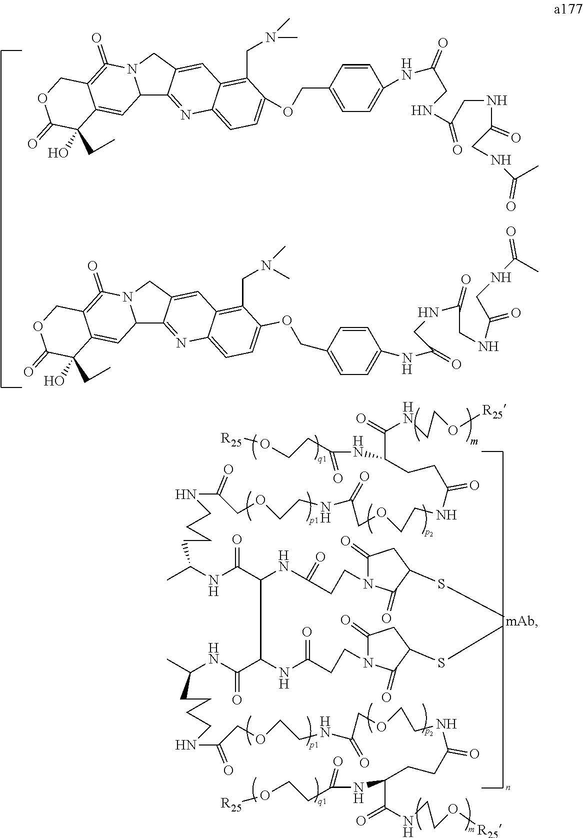 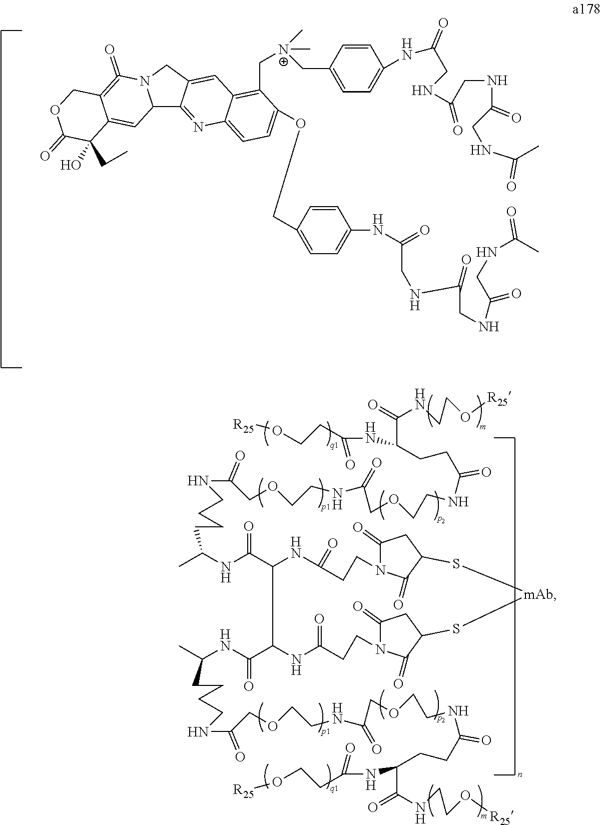 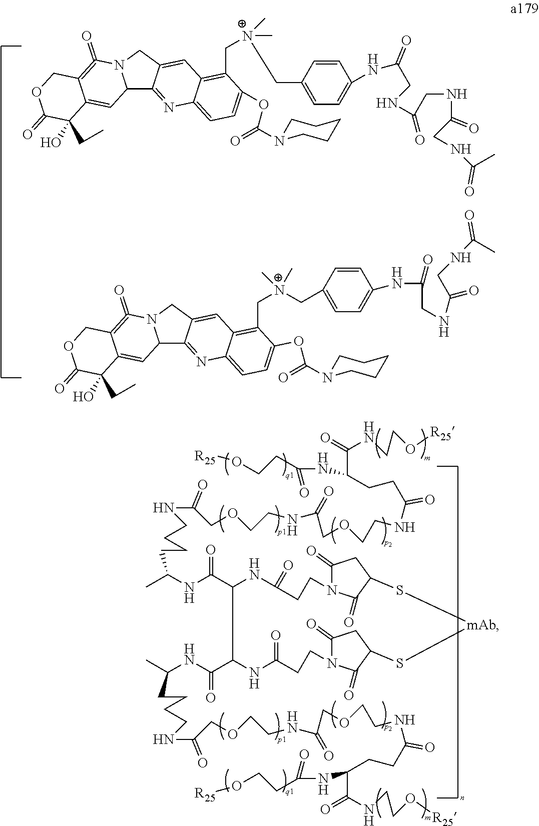 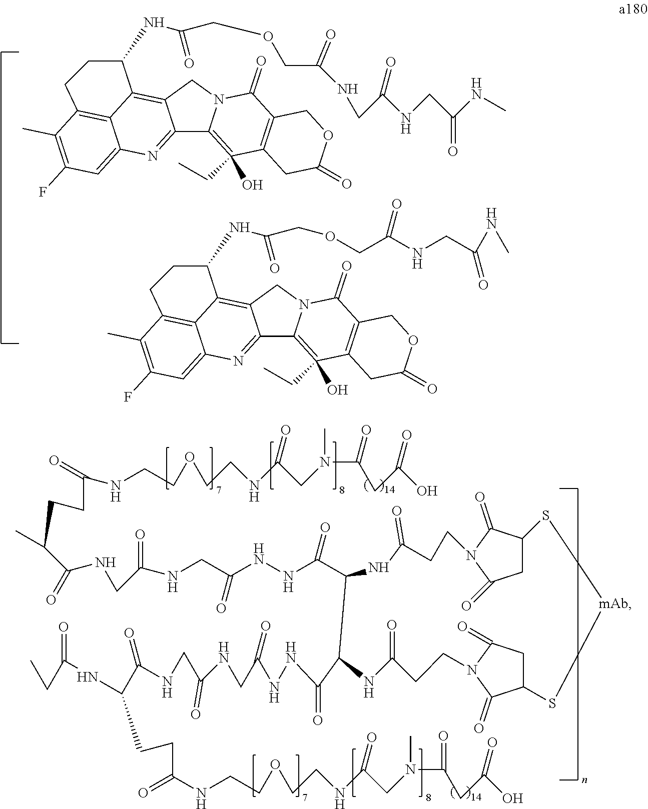 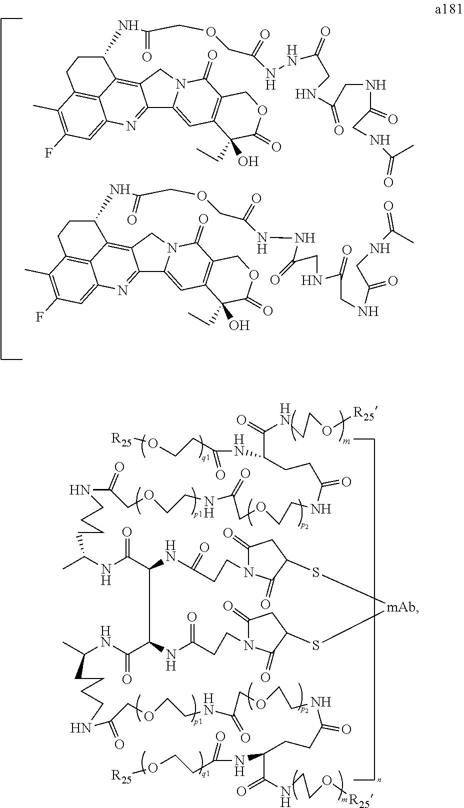 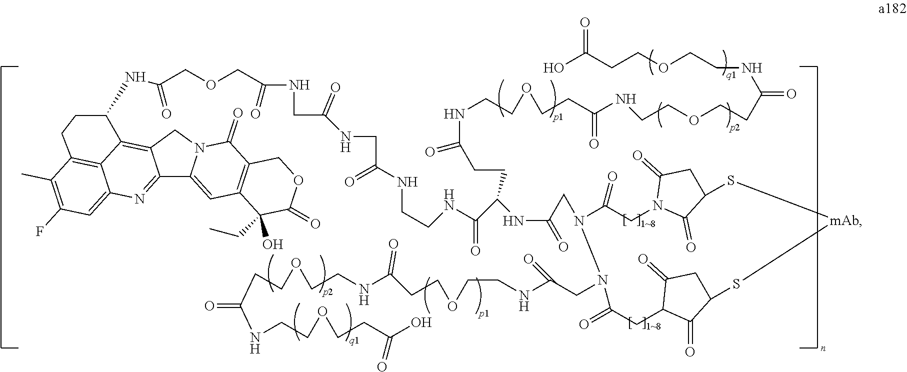 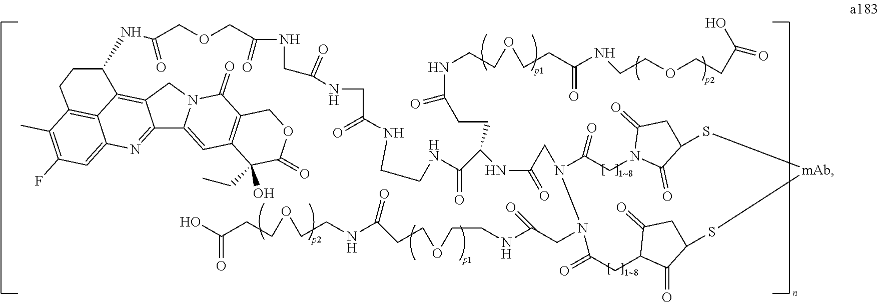 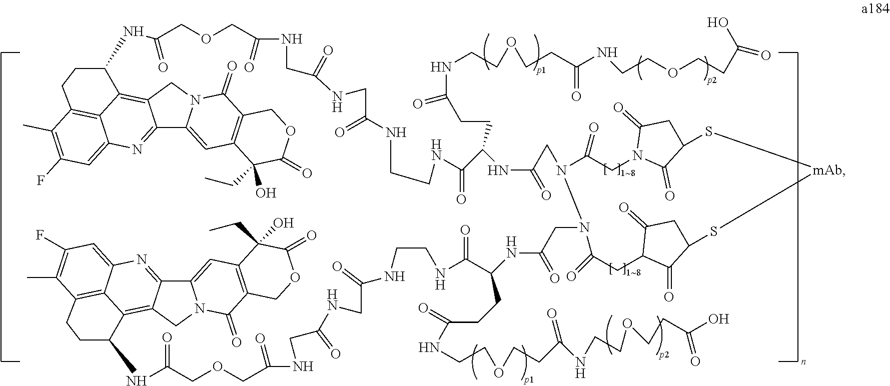 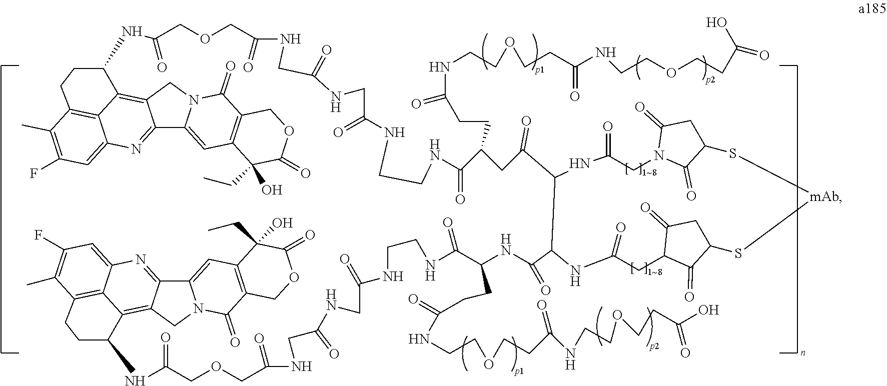 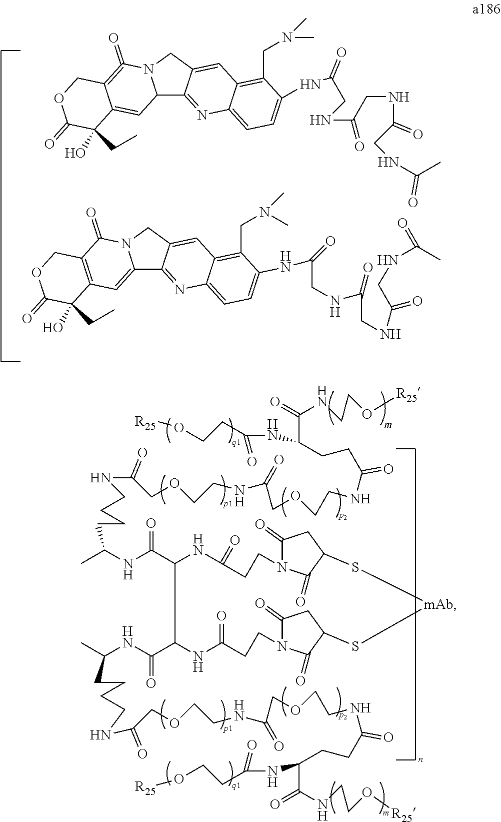 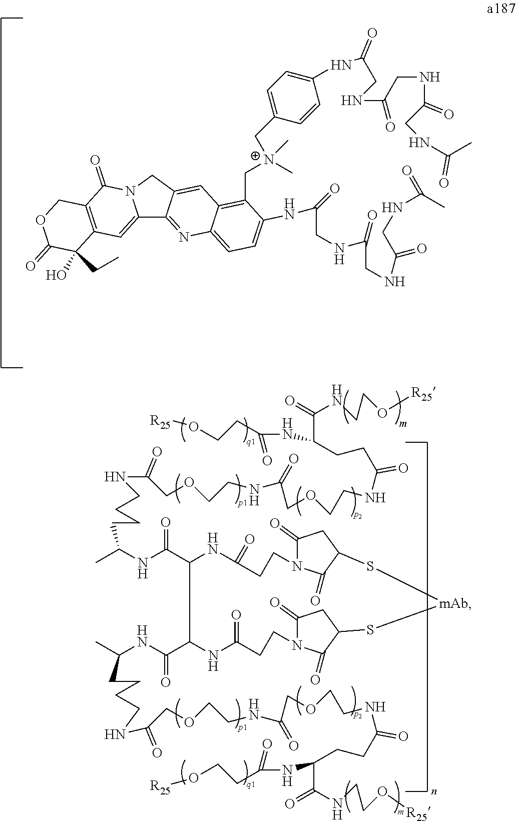 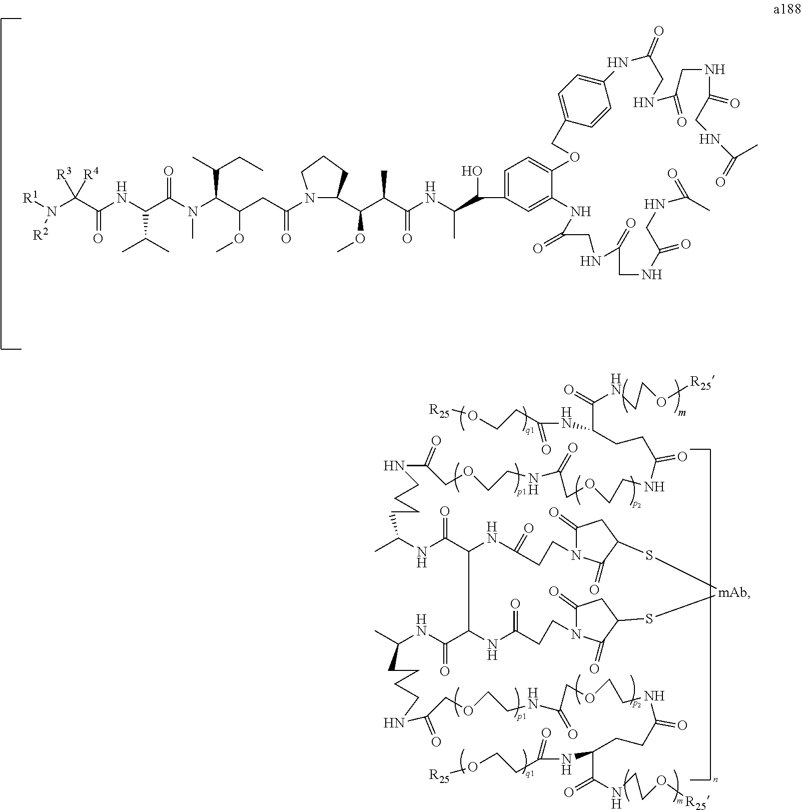 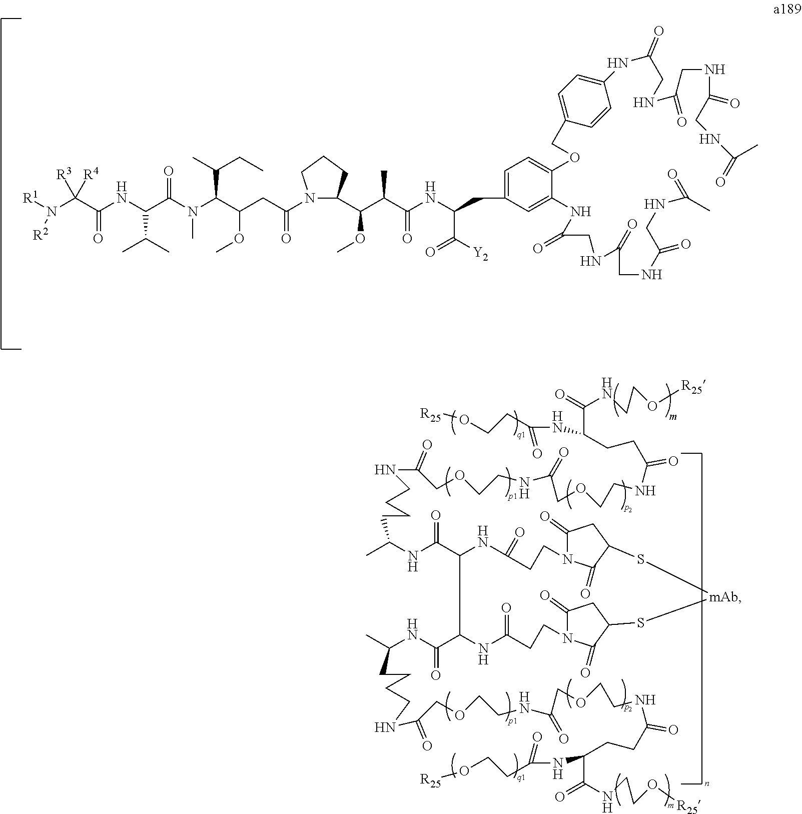 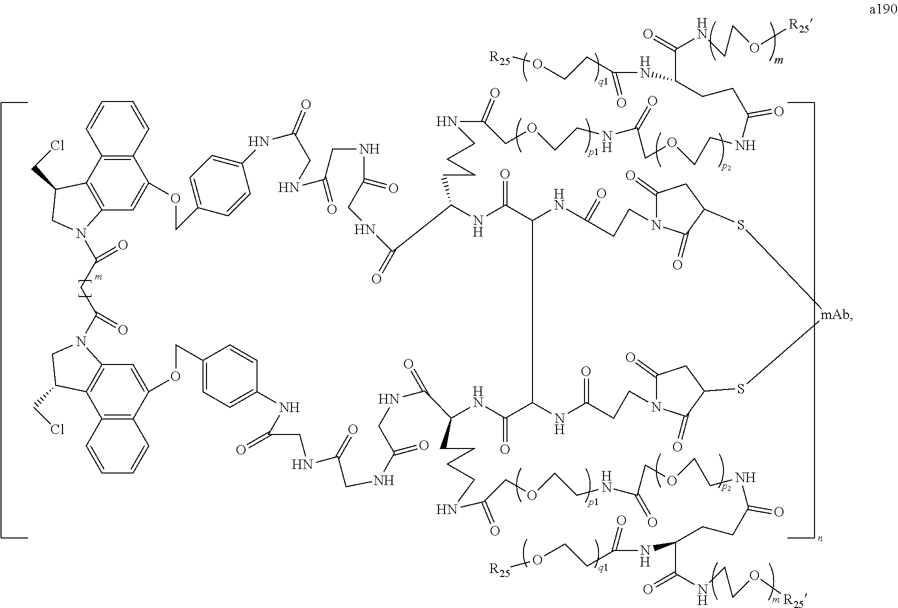 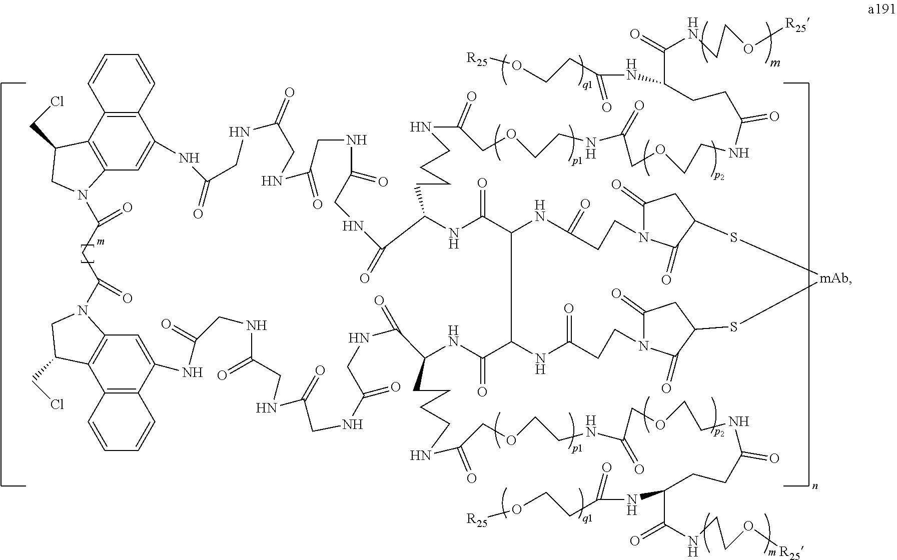 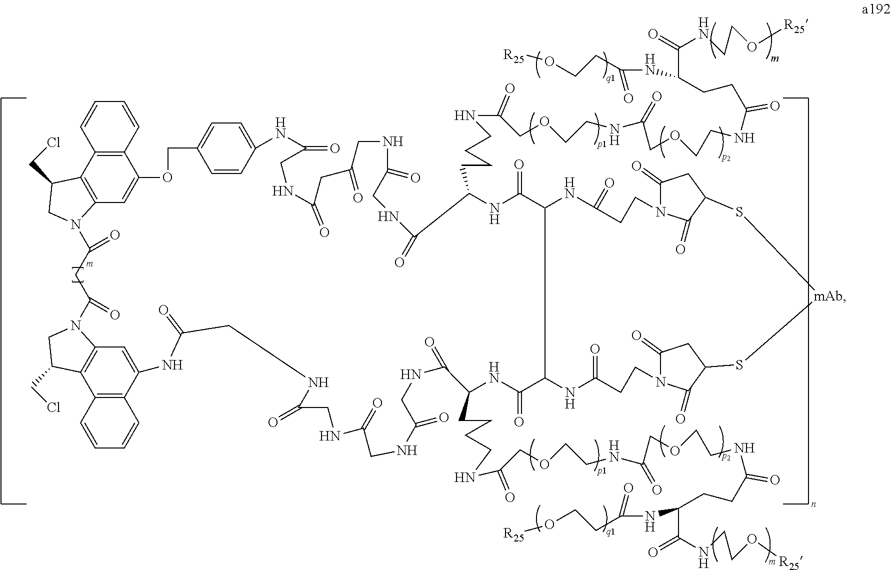 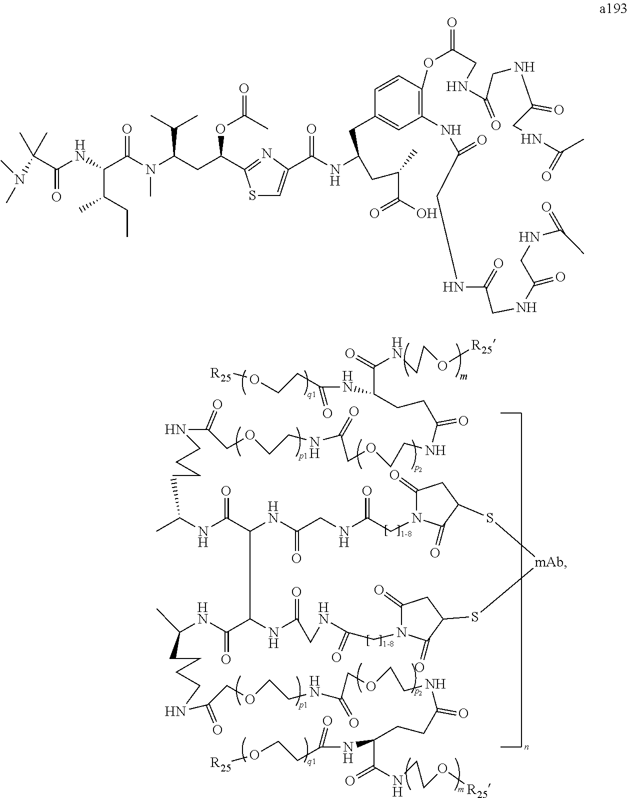 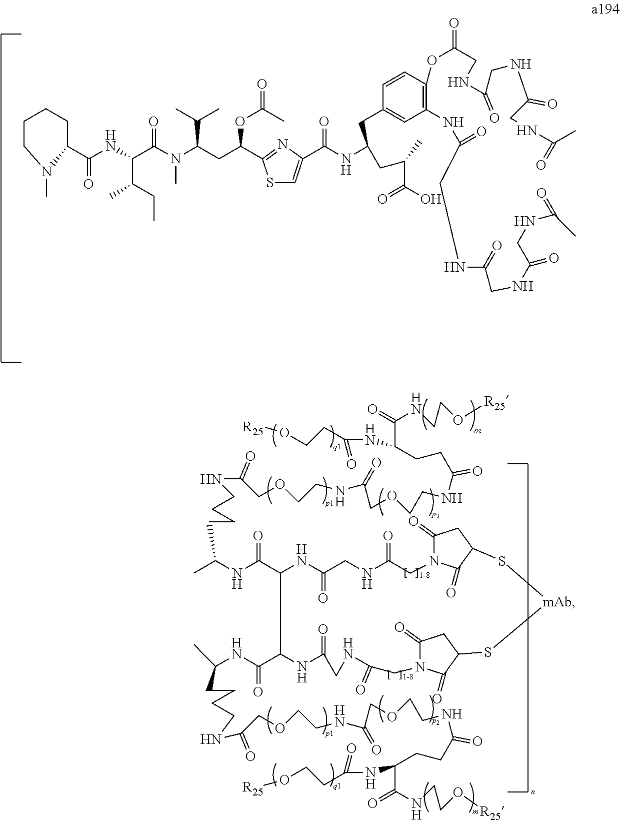 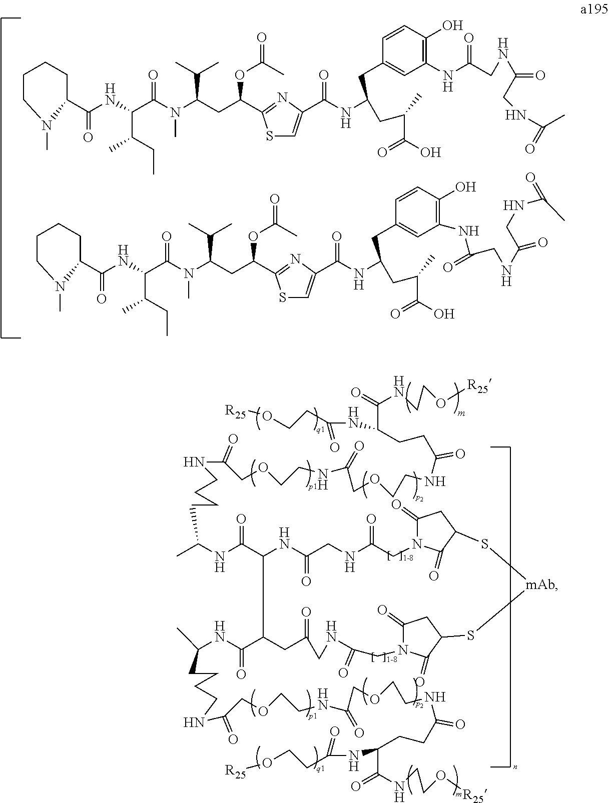 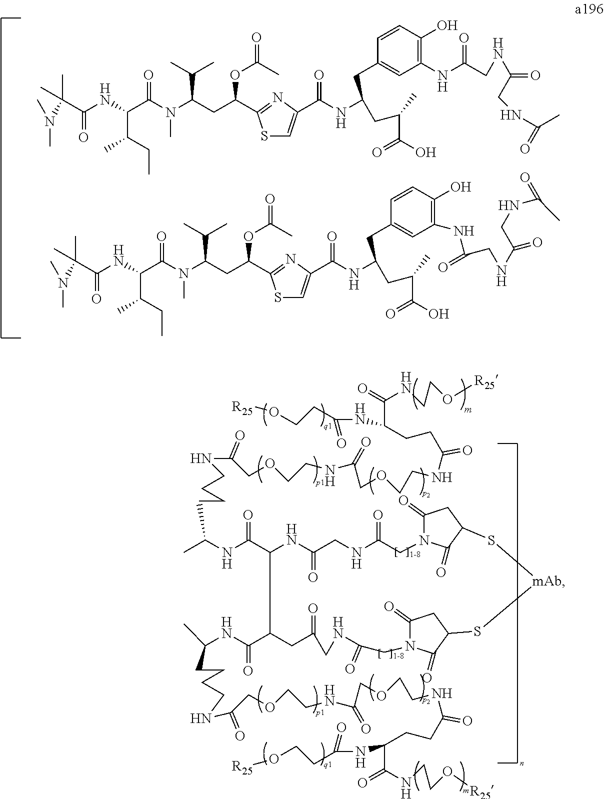 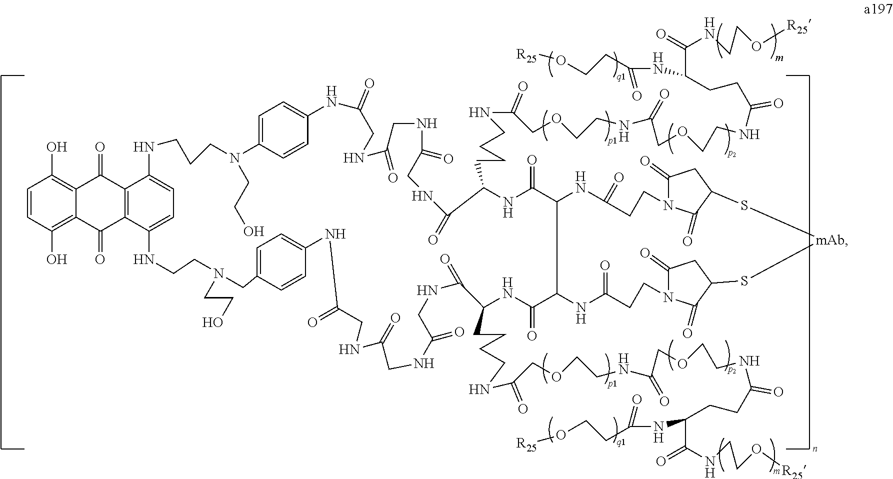 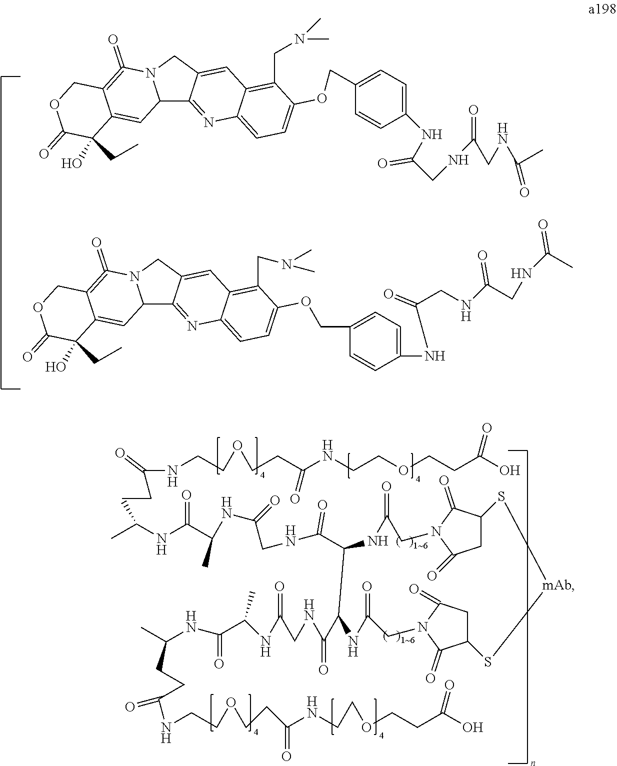 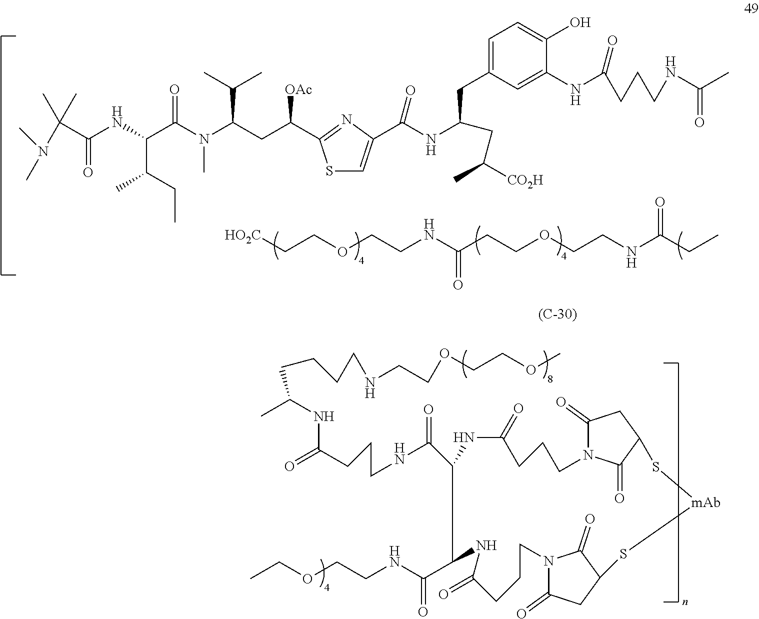 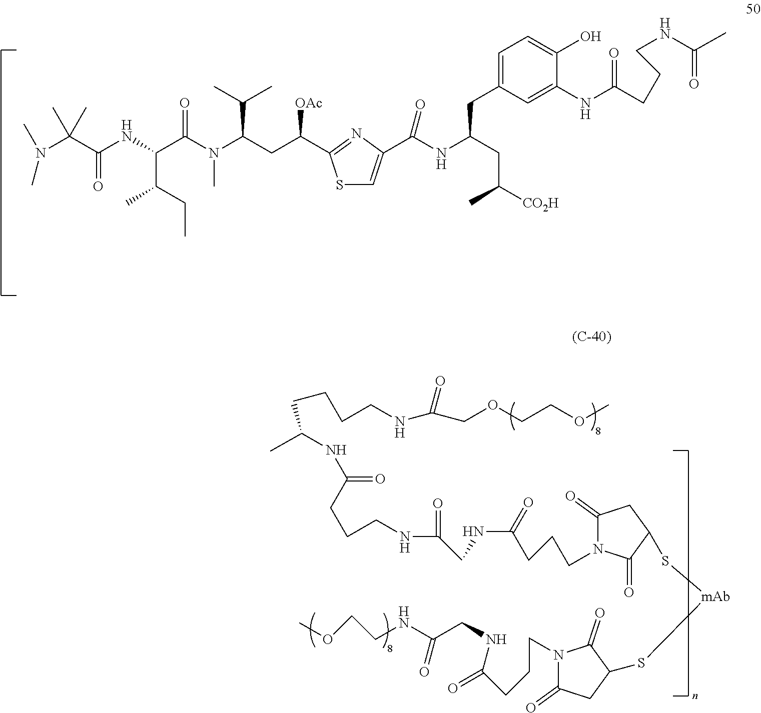 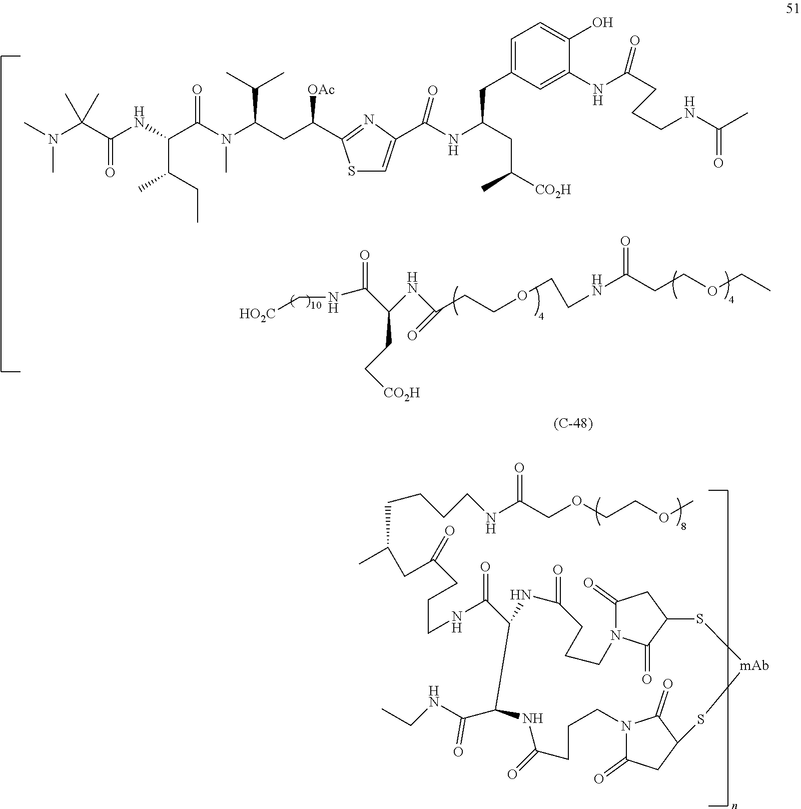 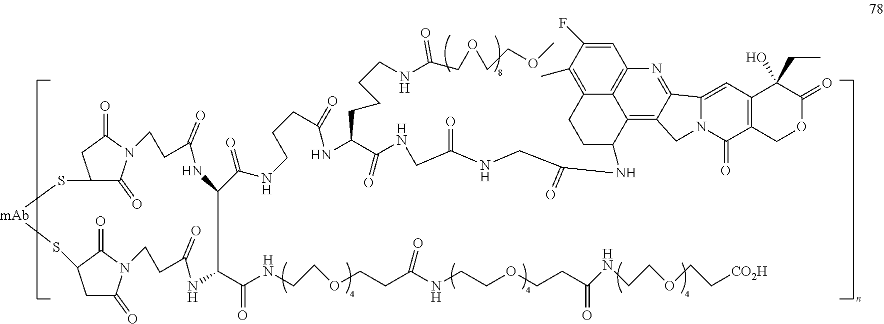 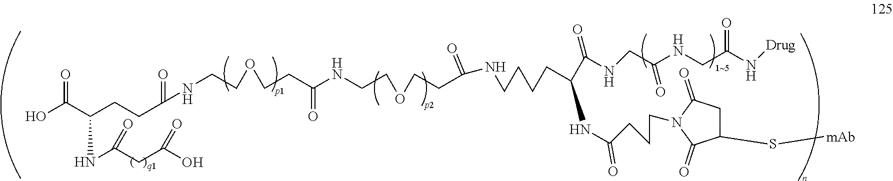 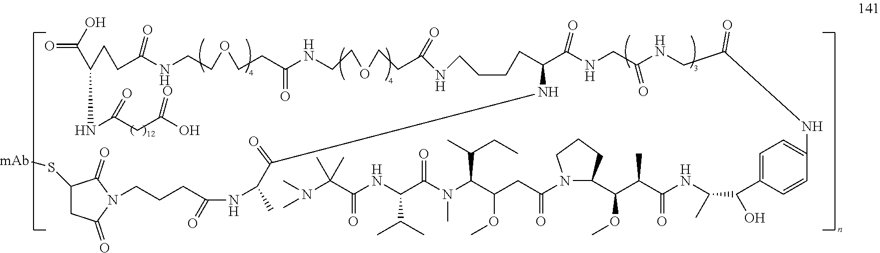 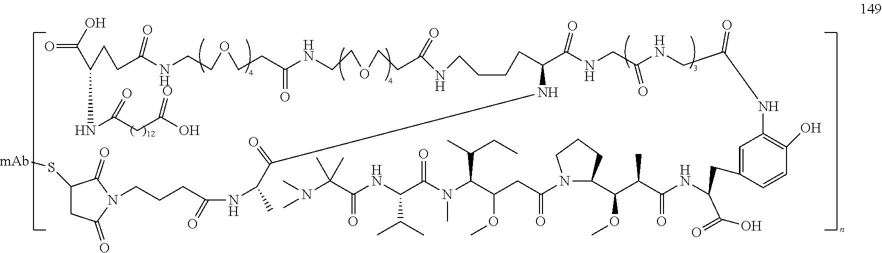 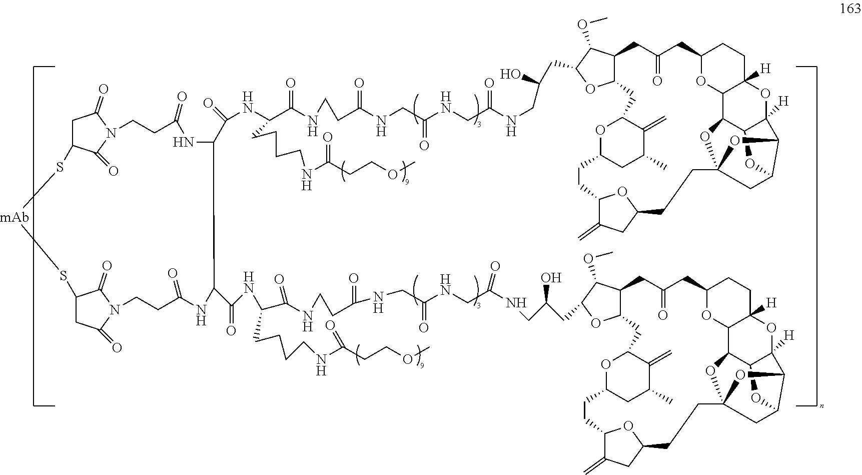 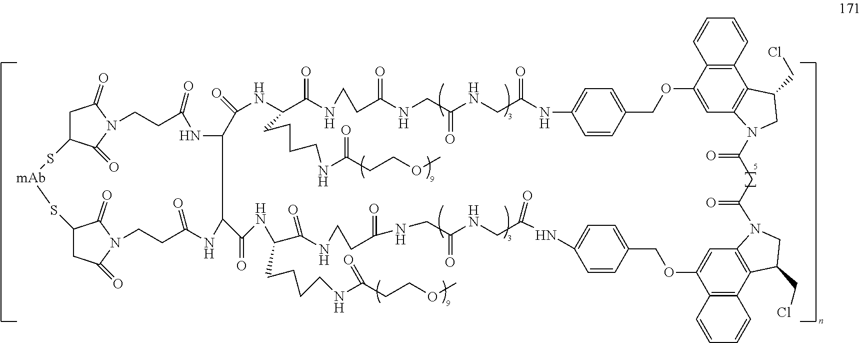 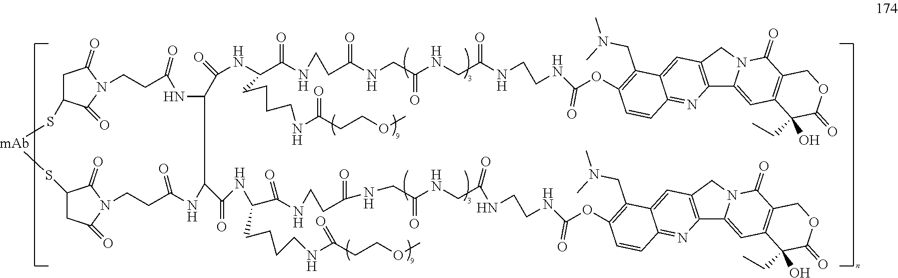 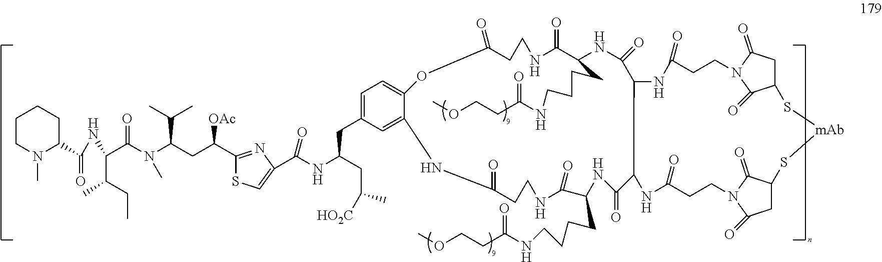 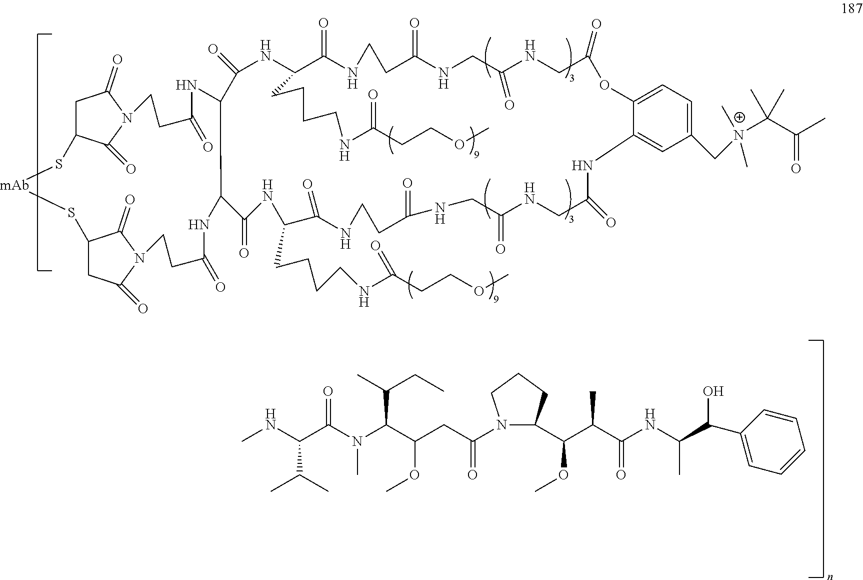 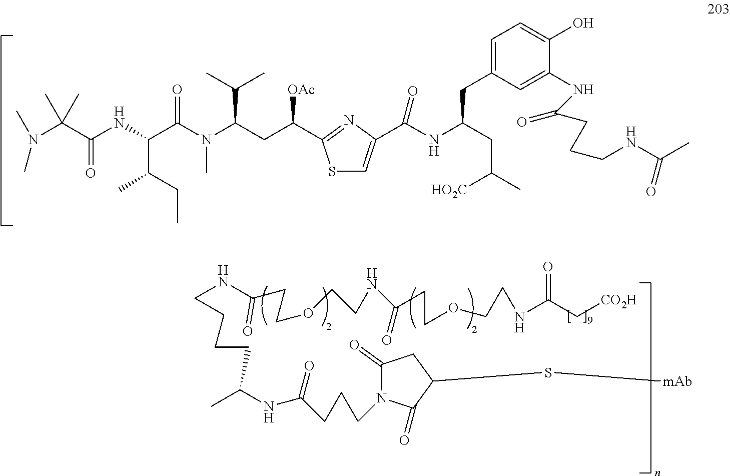 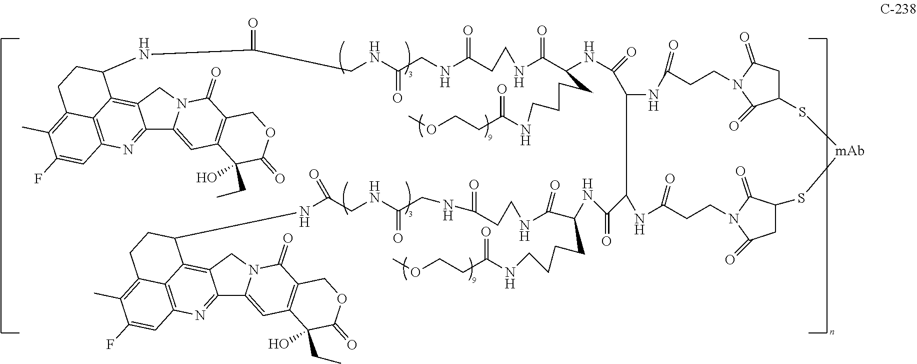 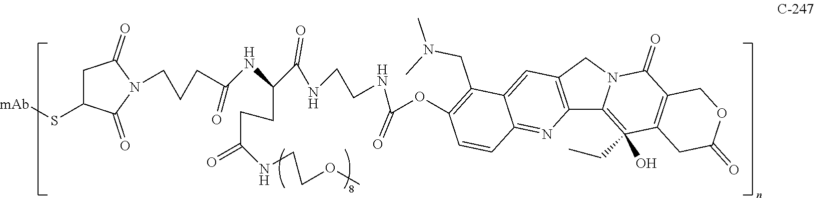 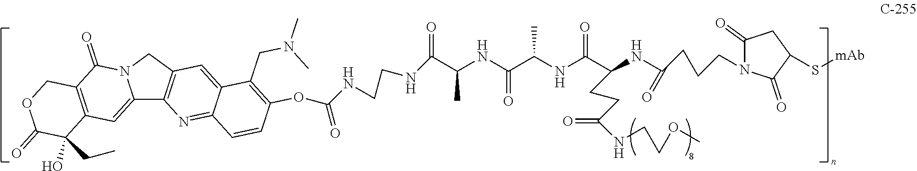 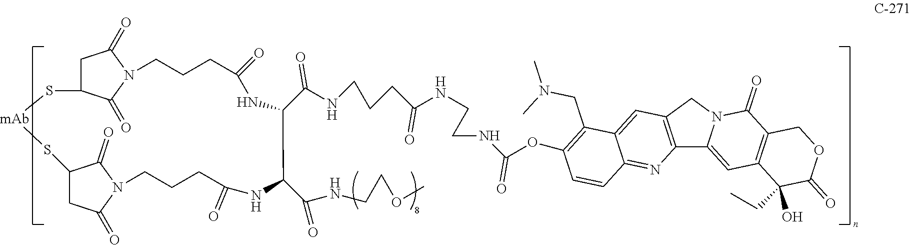 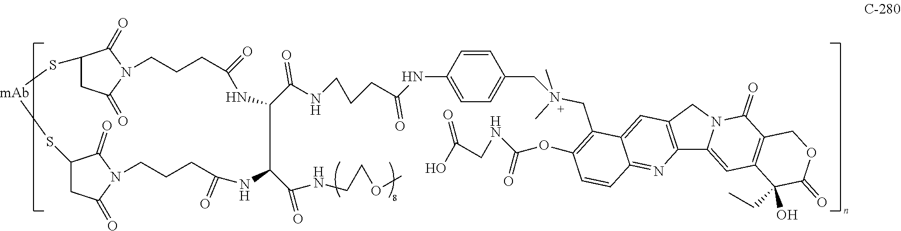 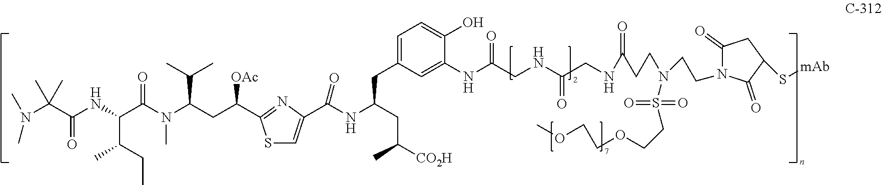 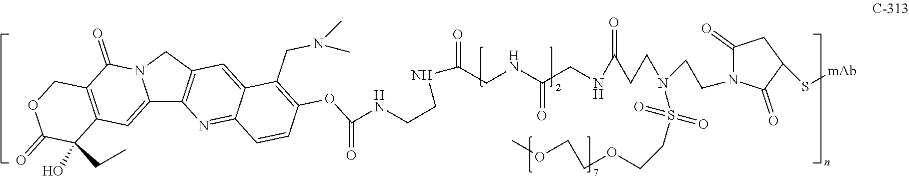 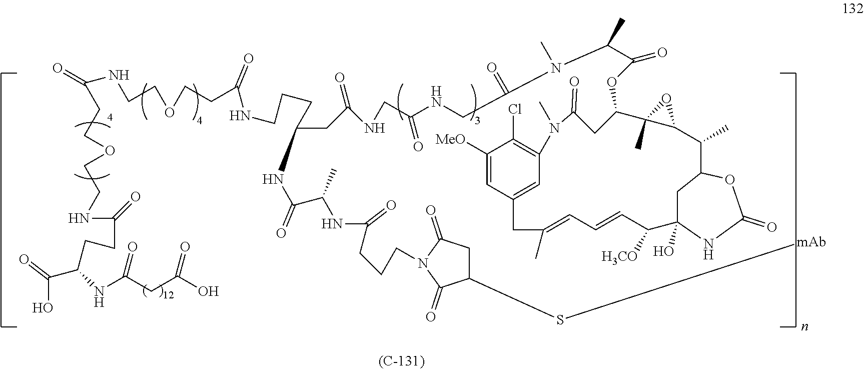 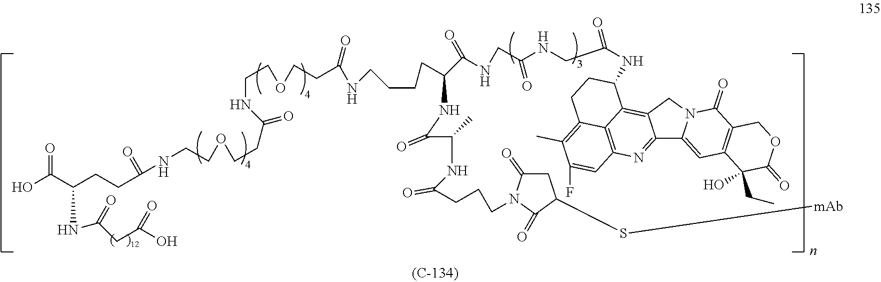 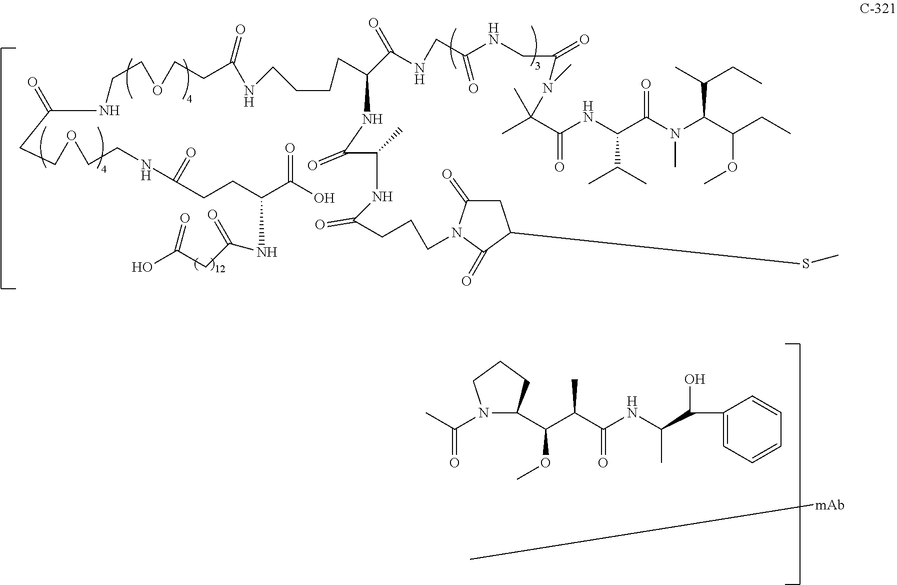 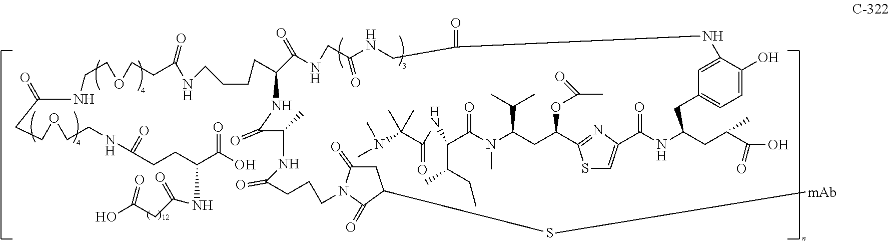
or a derivative thereof with one or more isotopes, or a pharmaceutically acceptable salt, hydrate, or hydrated salt thereof; or a polymorphic crystalline structure thereof; or an optical isomer, racemate, diastereomer or enantiomer thereof; wherein R¹, R², R³, R⁴, R⁵, R₄, R₅, R₇, R₈, R₉, R₁₀, X₁, X₂, X₃, X₄, X₅, X₆, X₈, Y₁, Y₂, Y₃, Y₅, R₁₂, R_{12′}, R₁₃, R₁₃′, R₂₅, R₂₅′, p₁, p₂, q₁, q₂, m, m₁, n, and mAb are described the same in claim 22; Aa is natural or unnatural amino acid; r is 0-12; (Aa)r is a peptide containing the same or different sequence of amino acids when r>2; r=0 means (Aa)r absent.

31. The compound according to claim 24 having one of the following structures b001 to b197, 30, 40, 48, 77, 124, 140, 148, 162, 170, 173, 178, 186, 202, 238, 247, 255, 271, 279, 312, 313, 131, 134, 321, and 322 illustrated below: 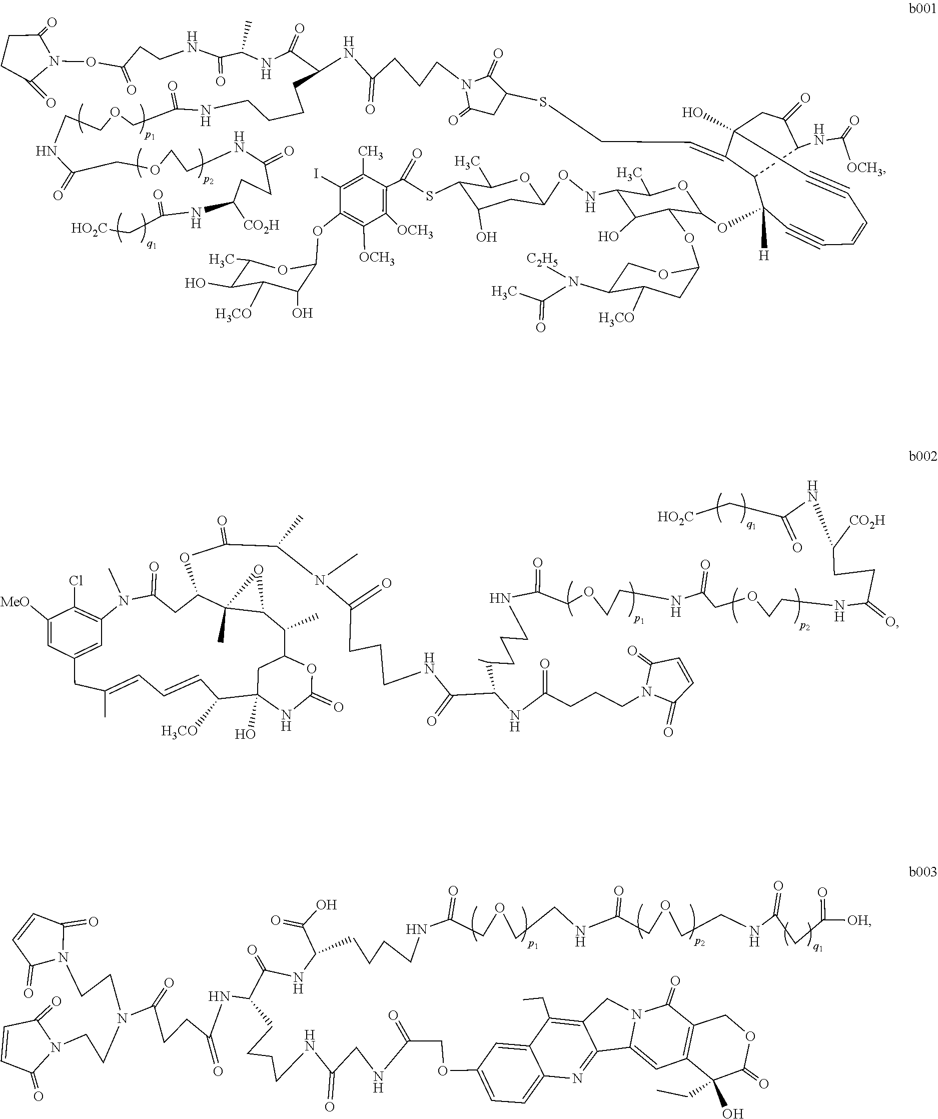 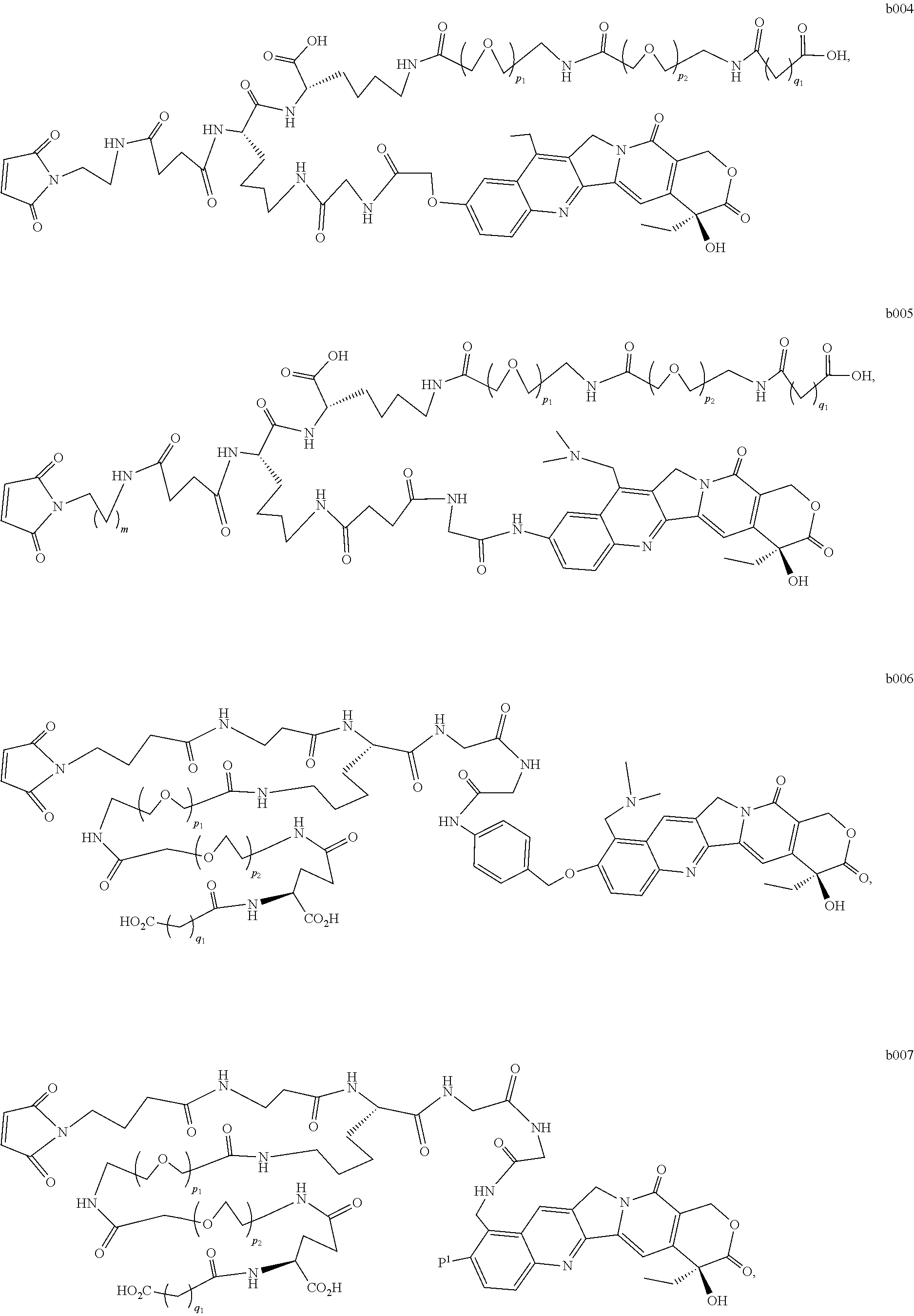 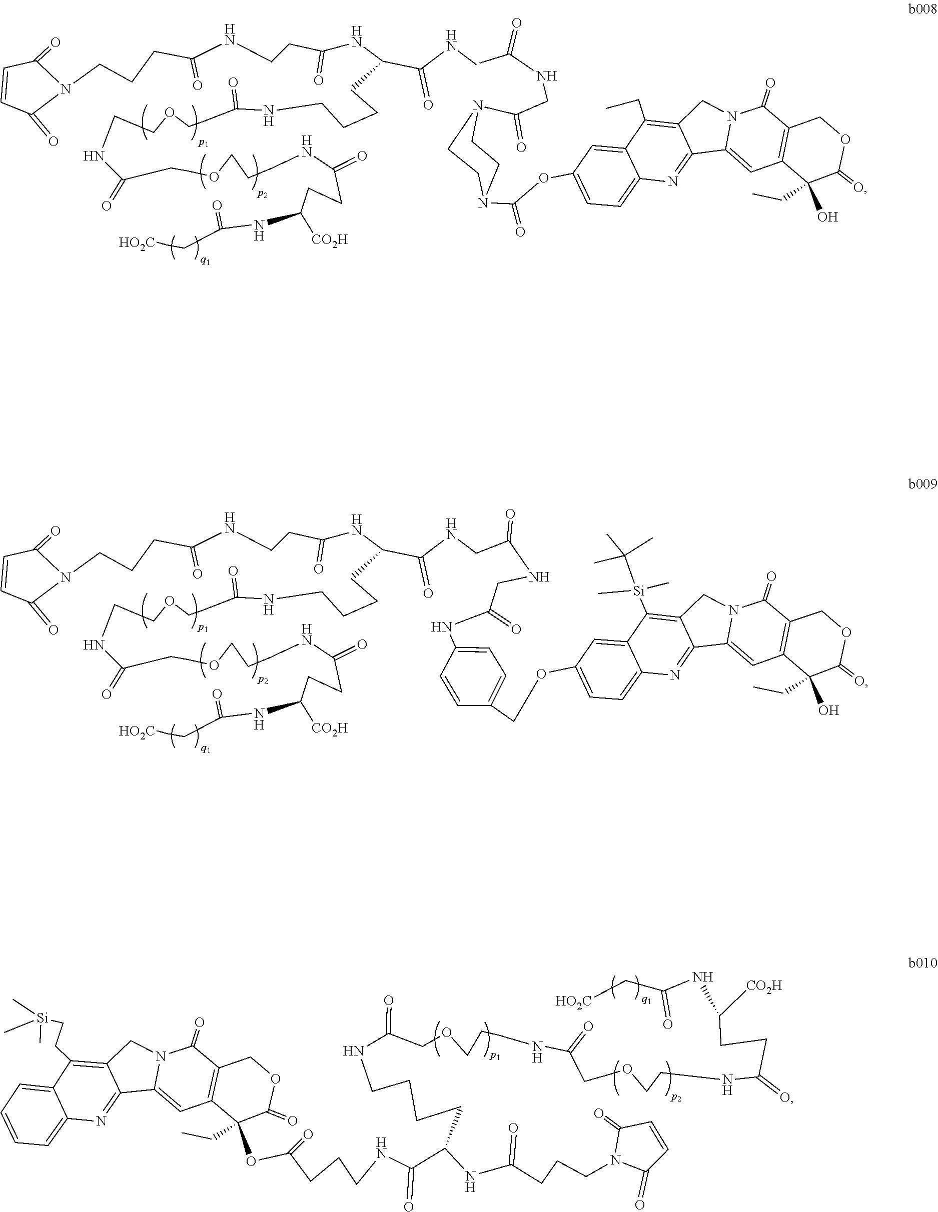 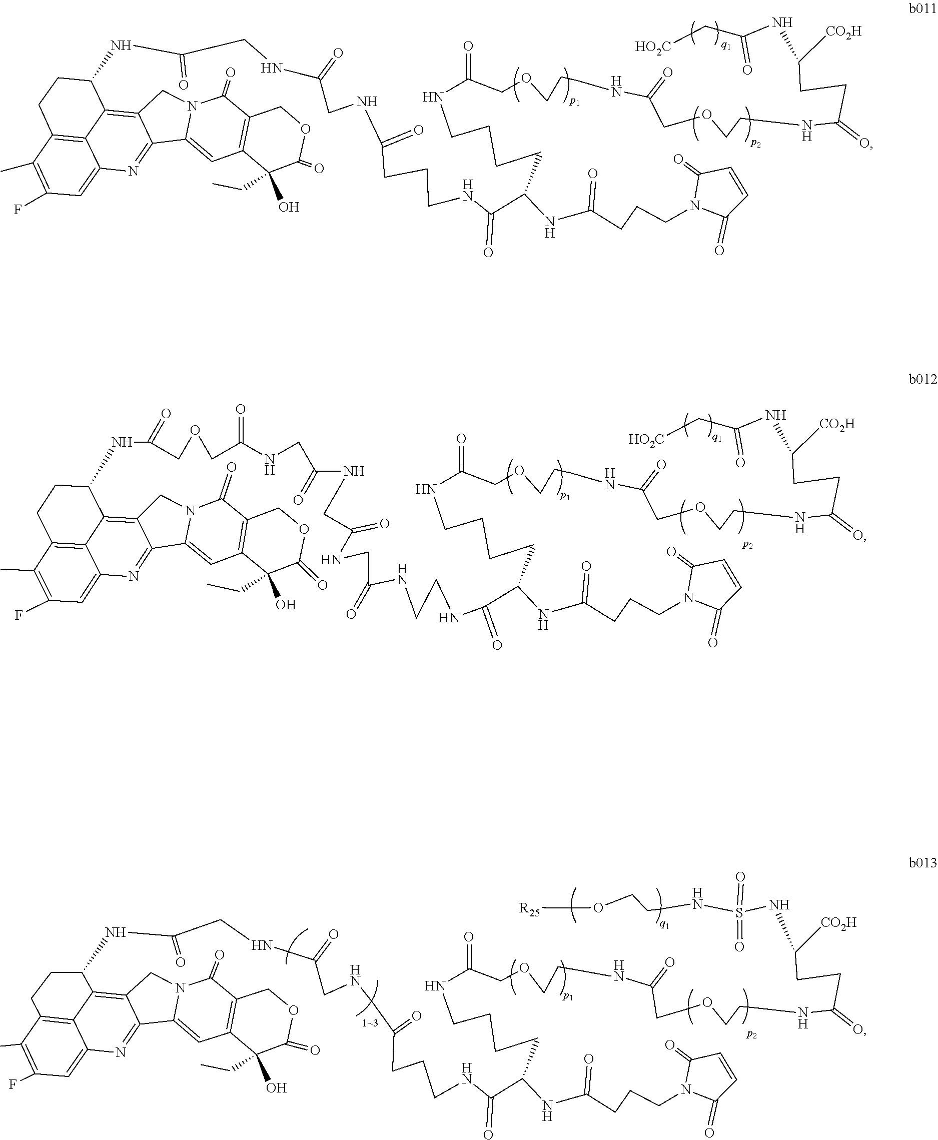 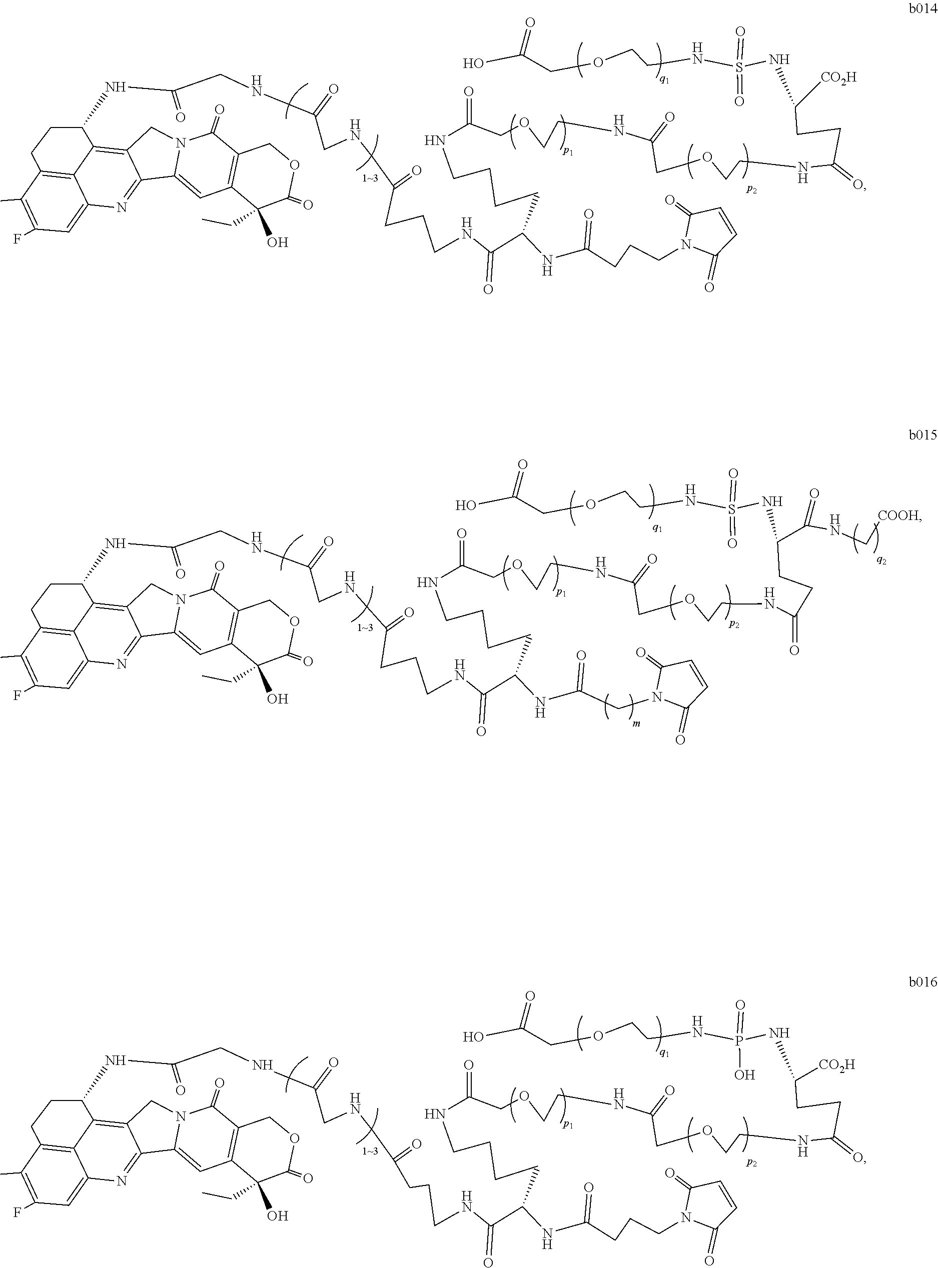 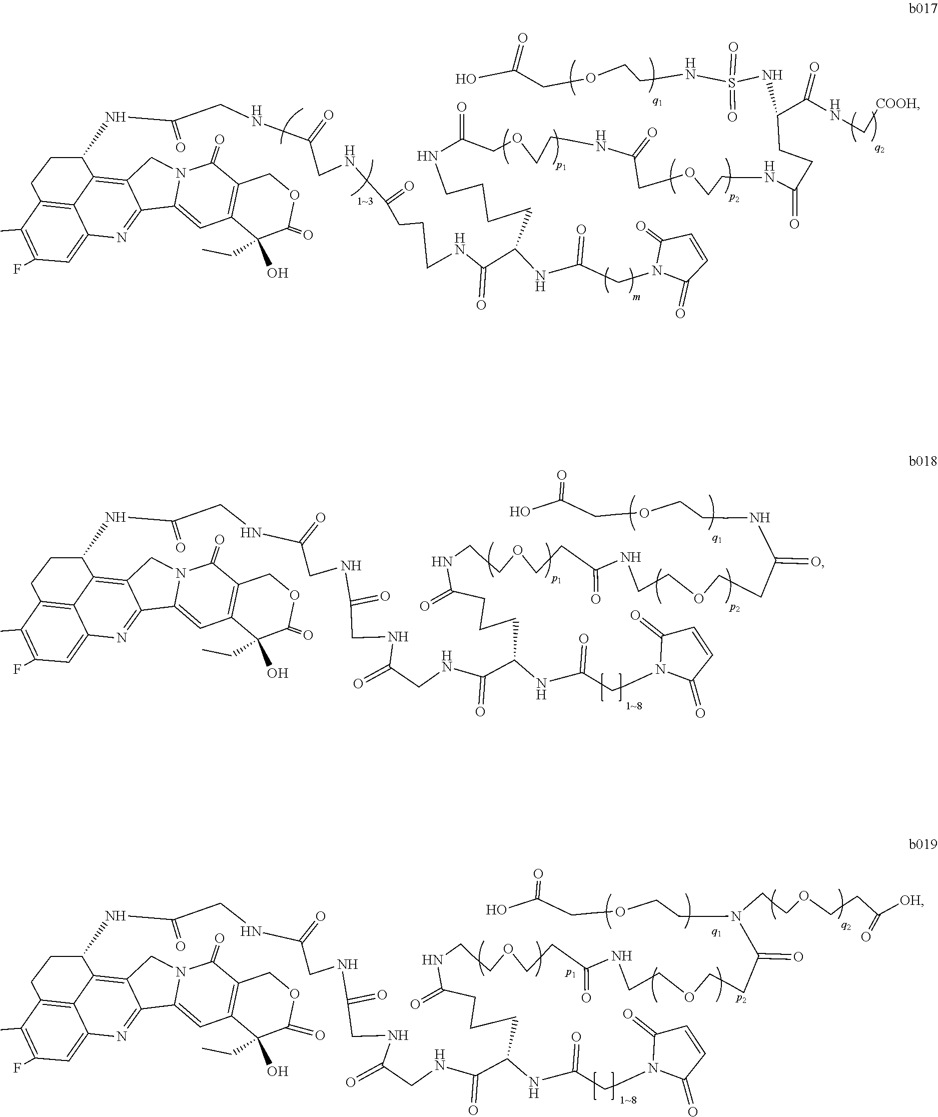 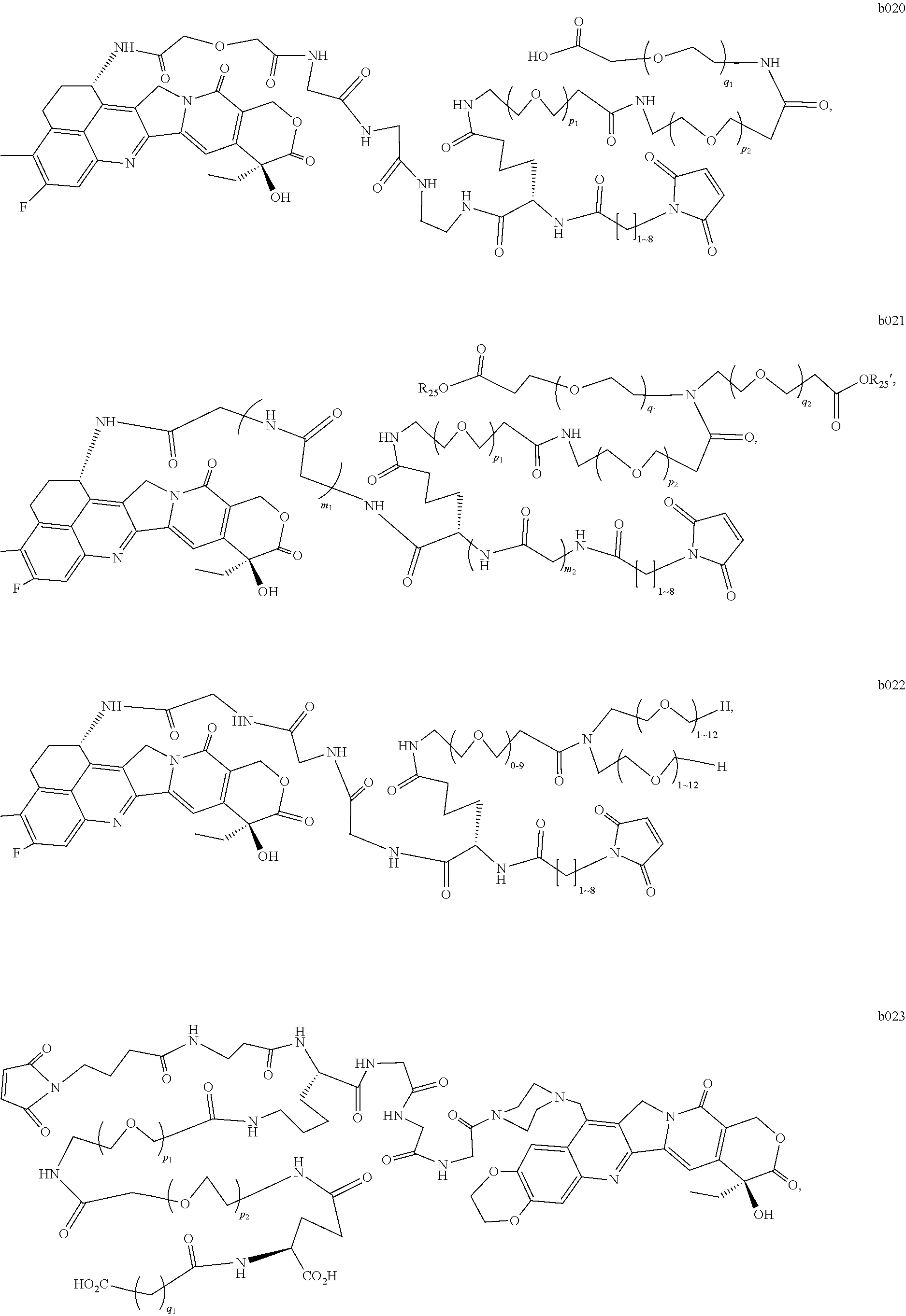 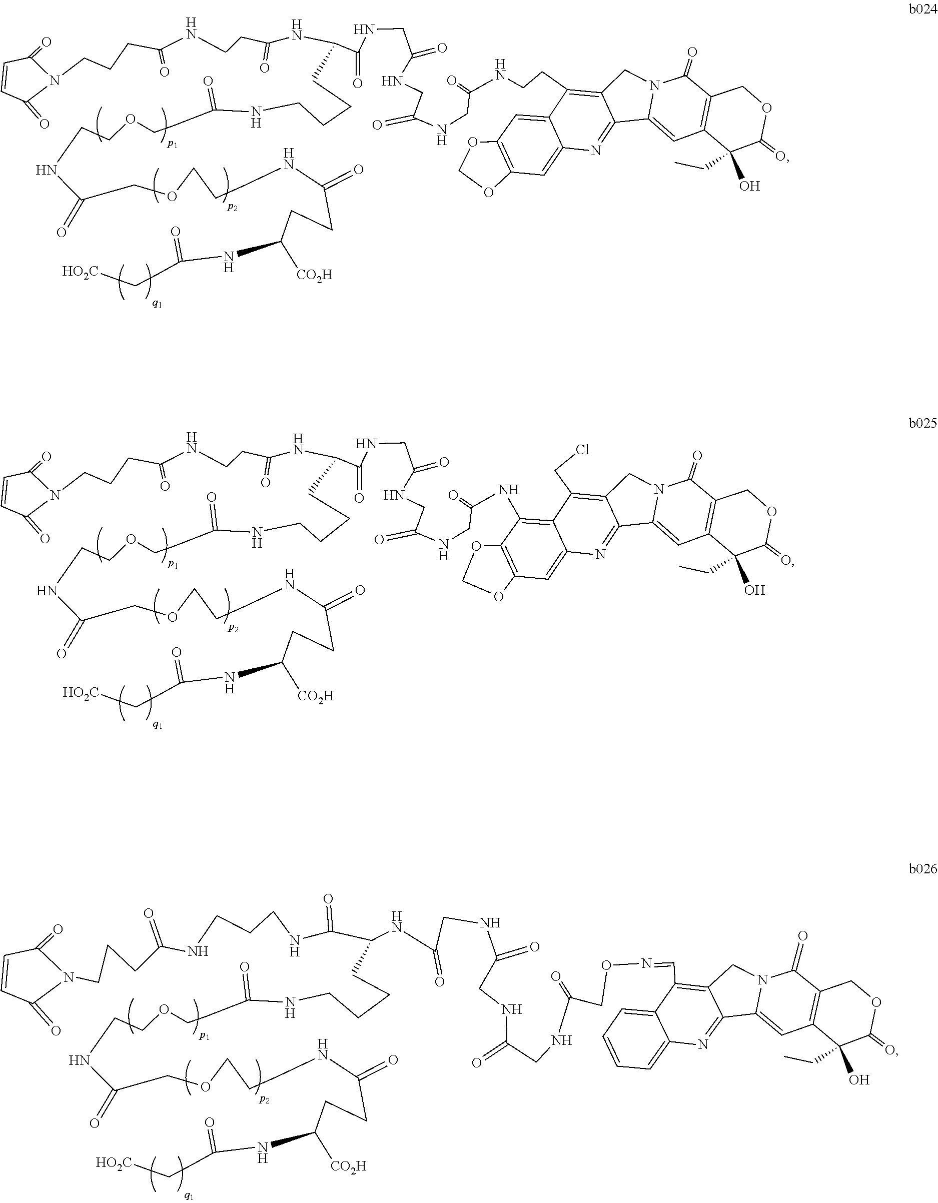 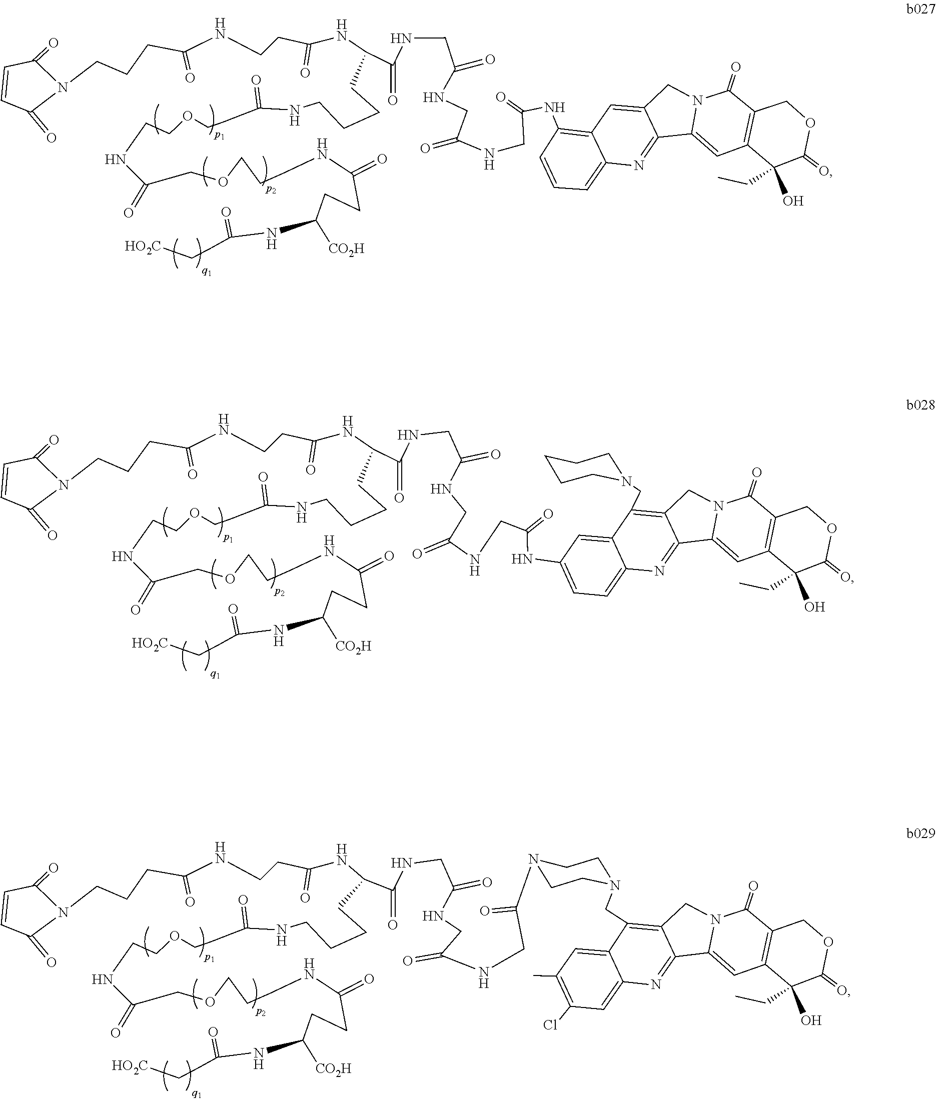 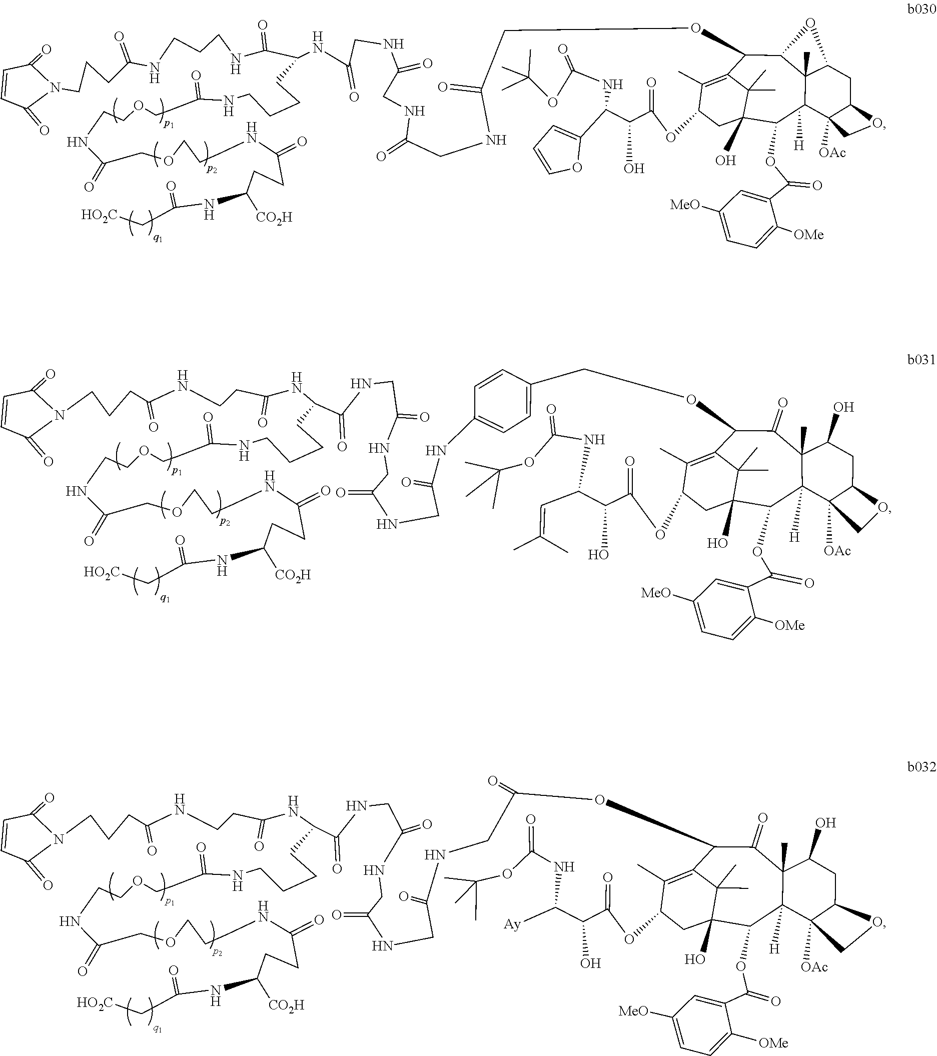 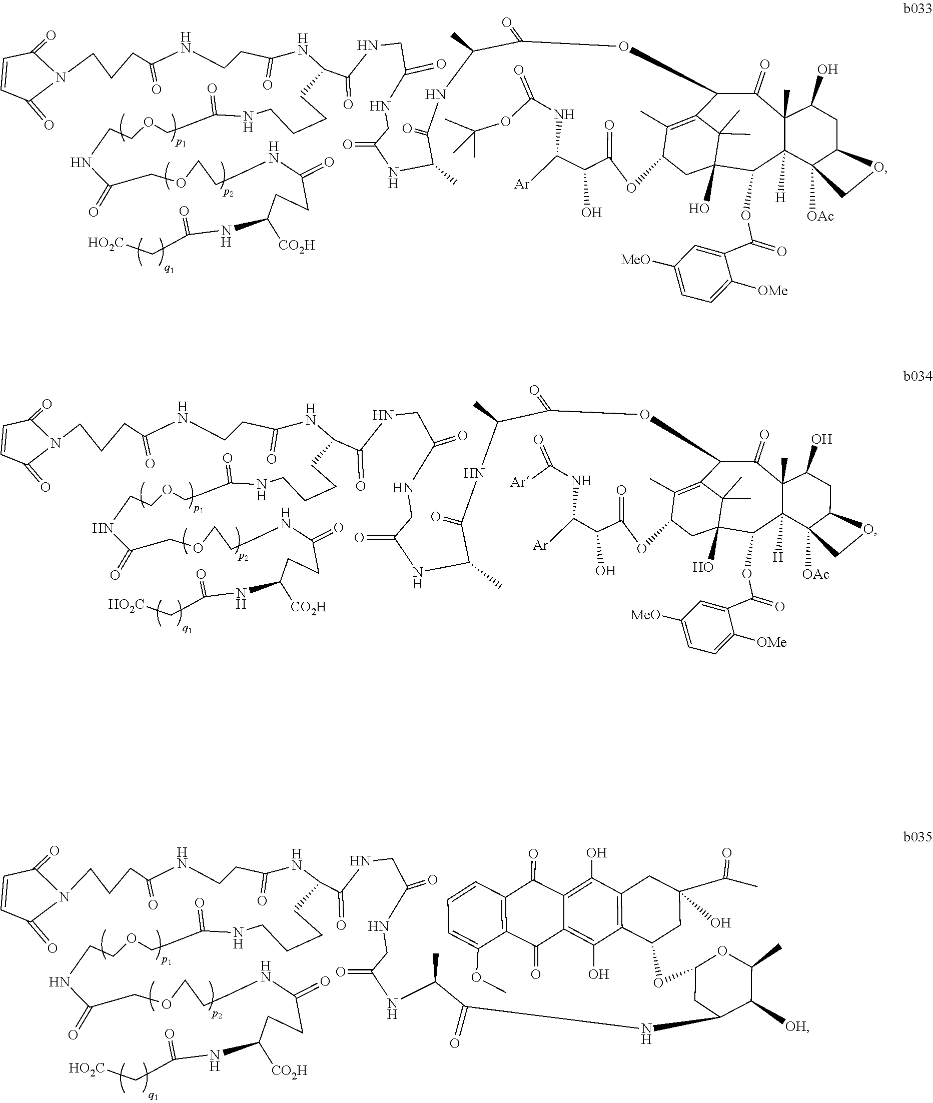 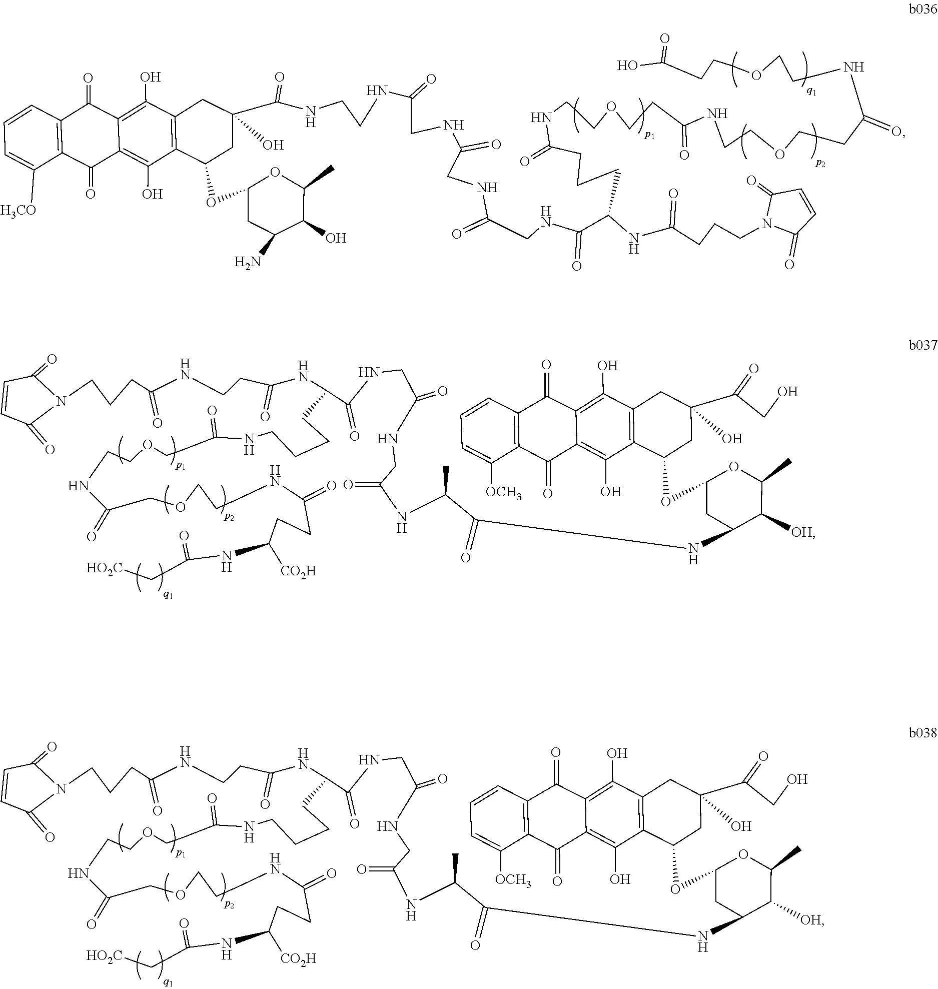 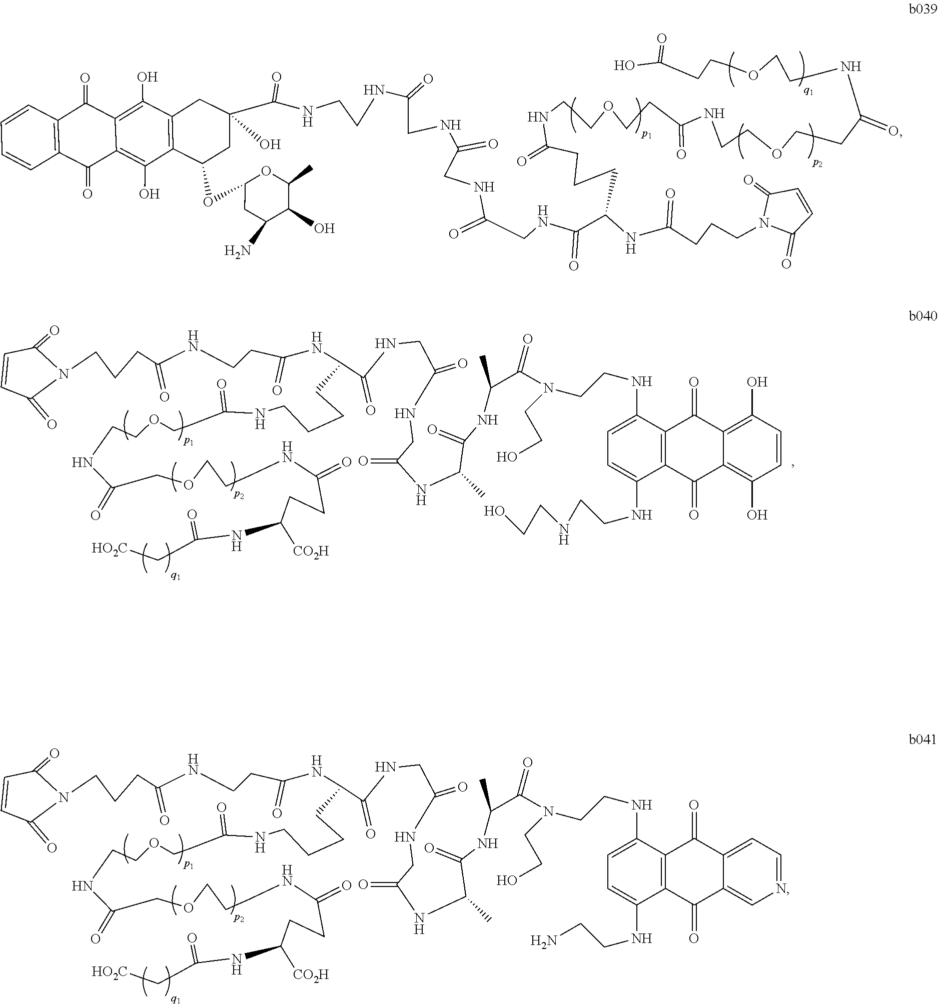 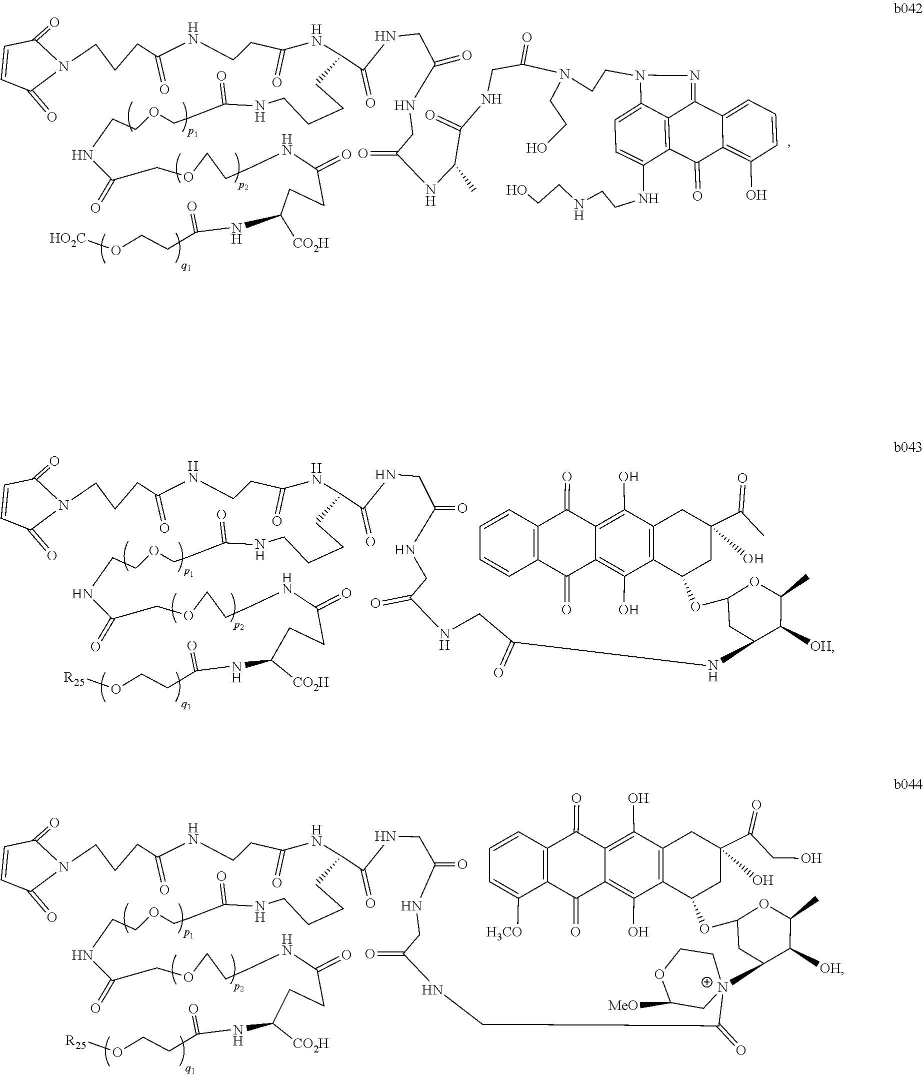 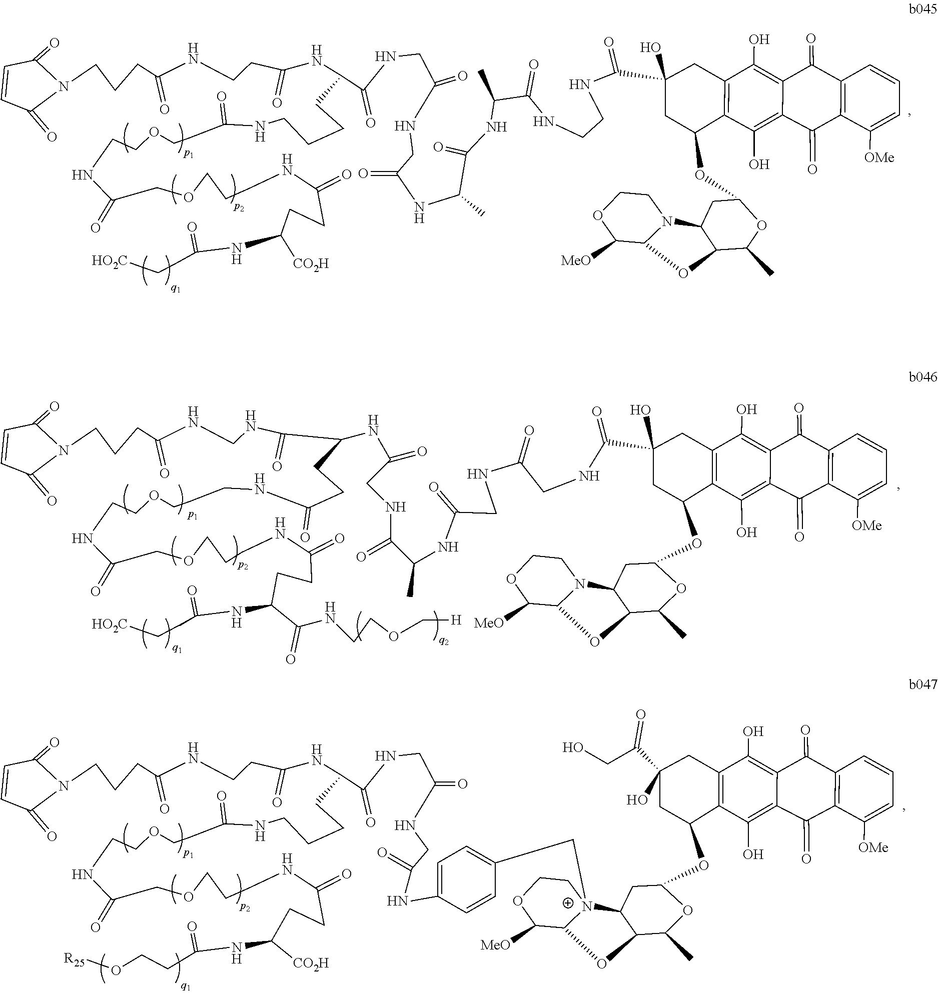 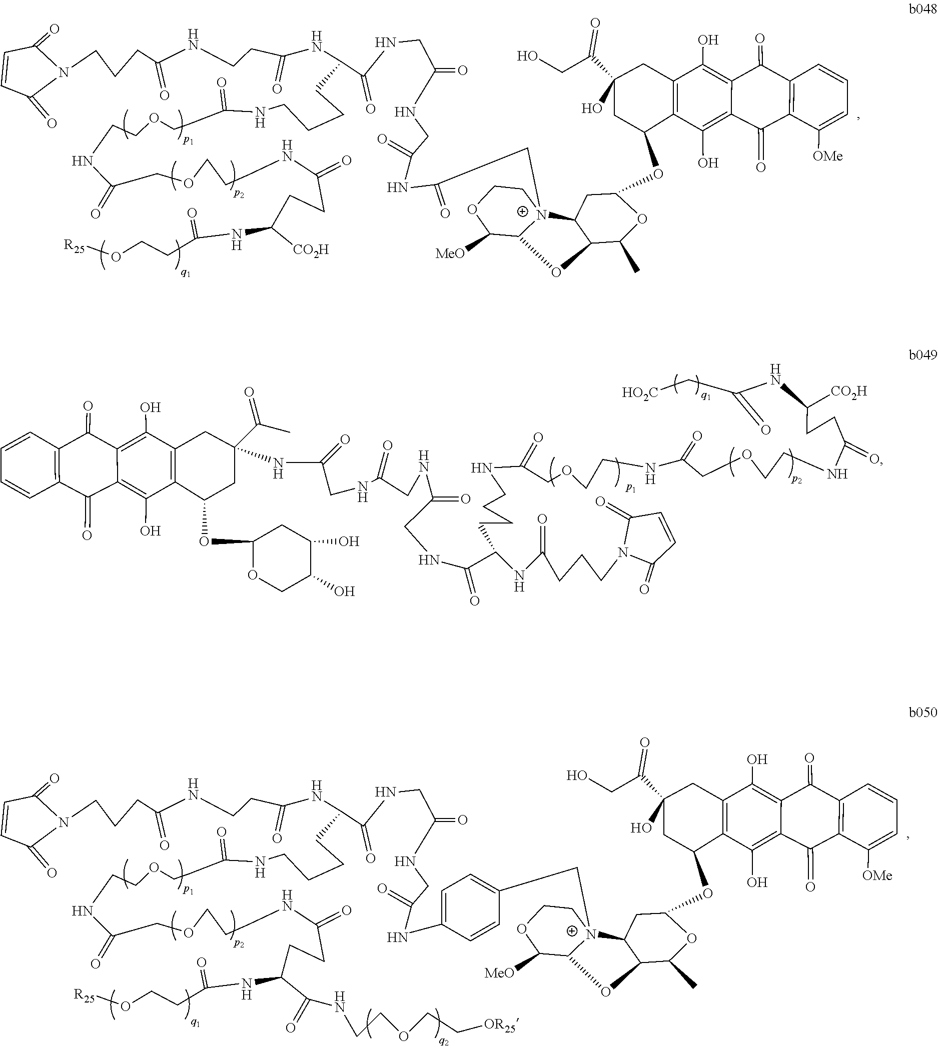 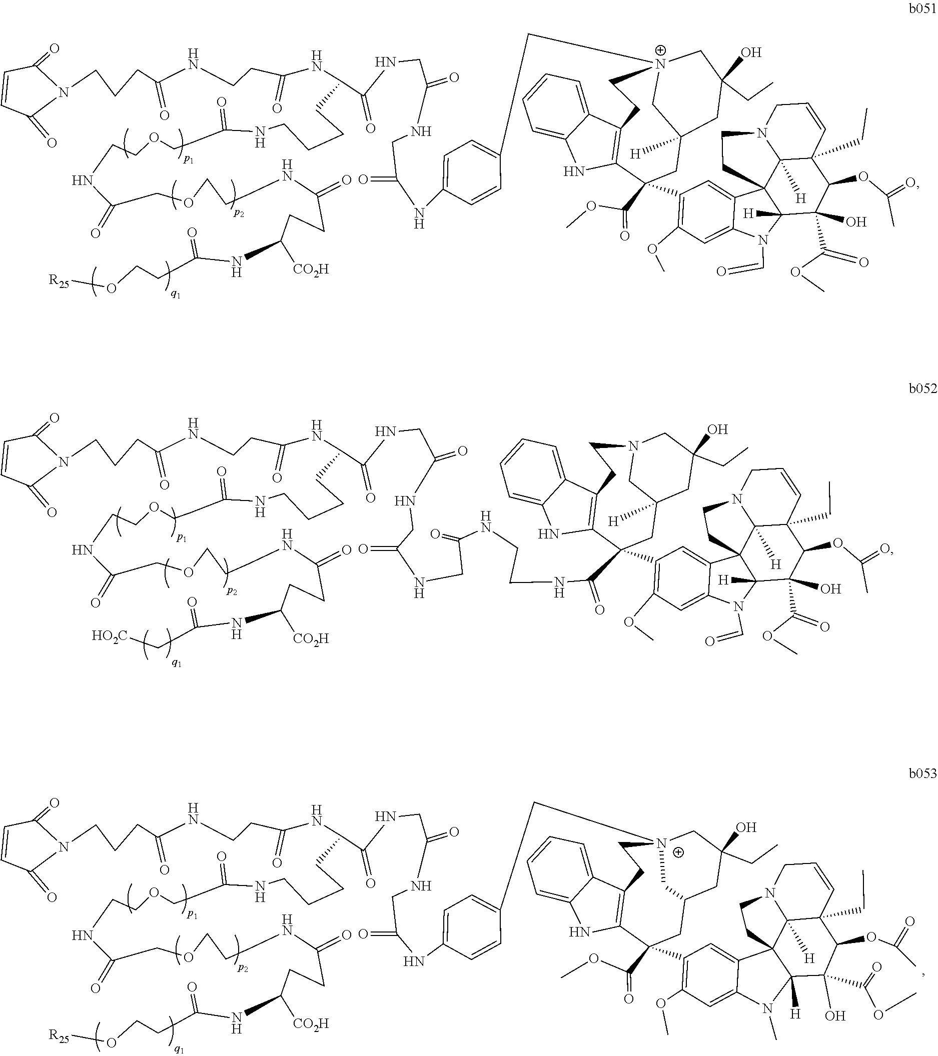 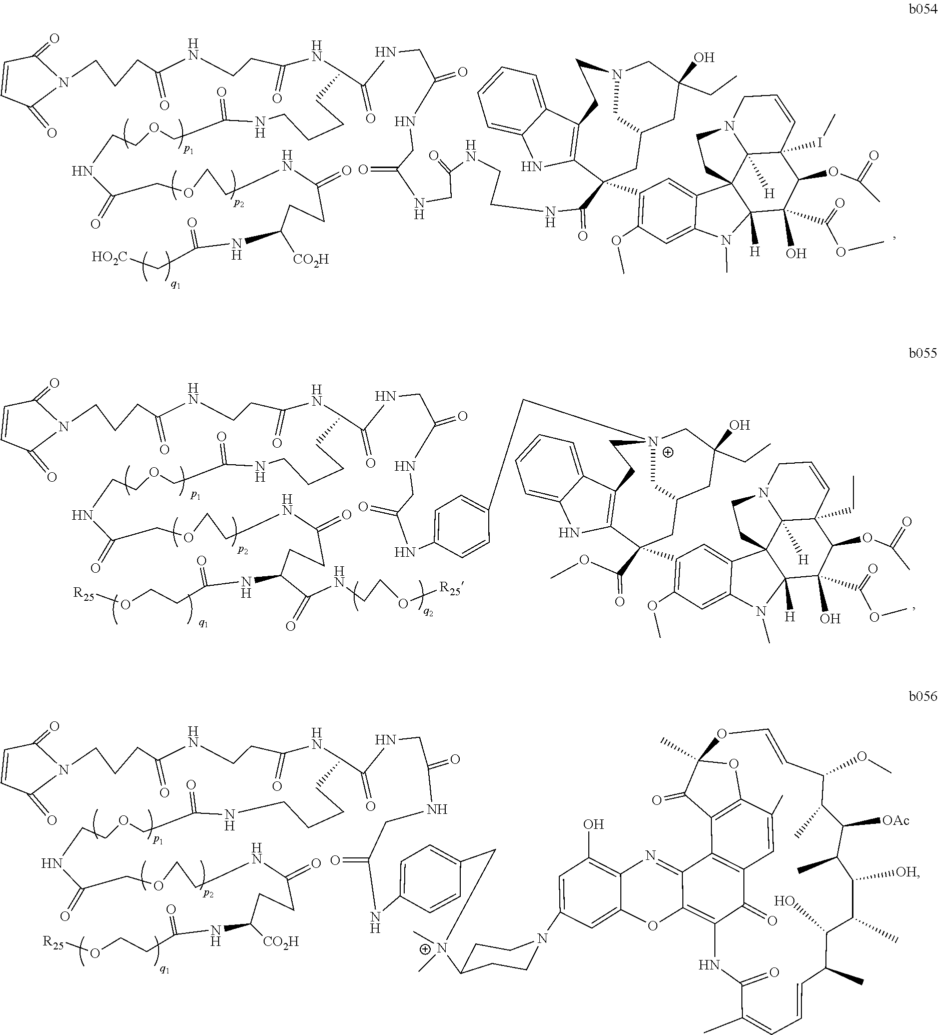 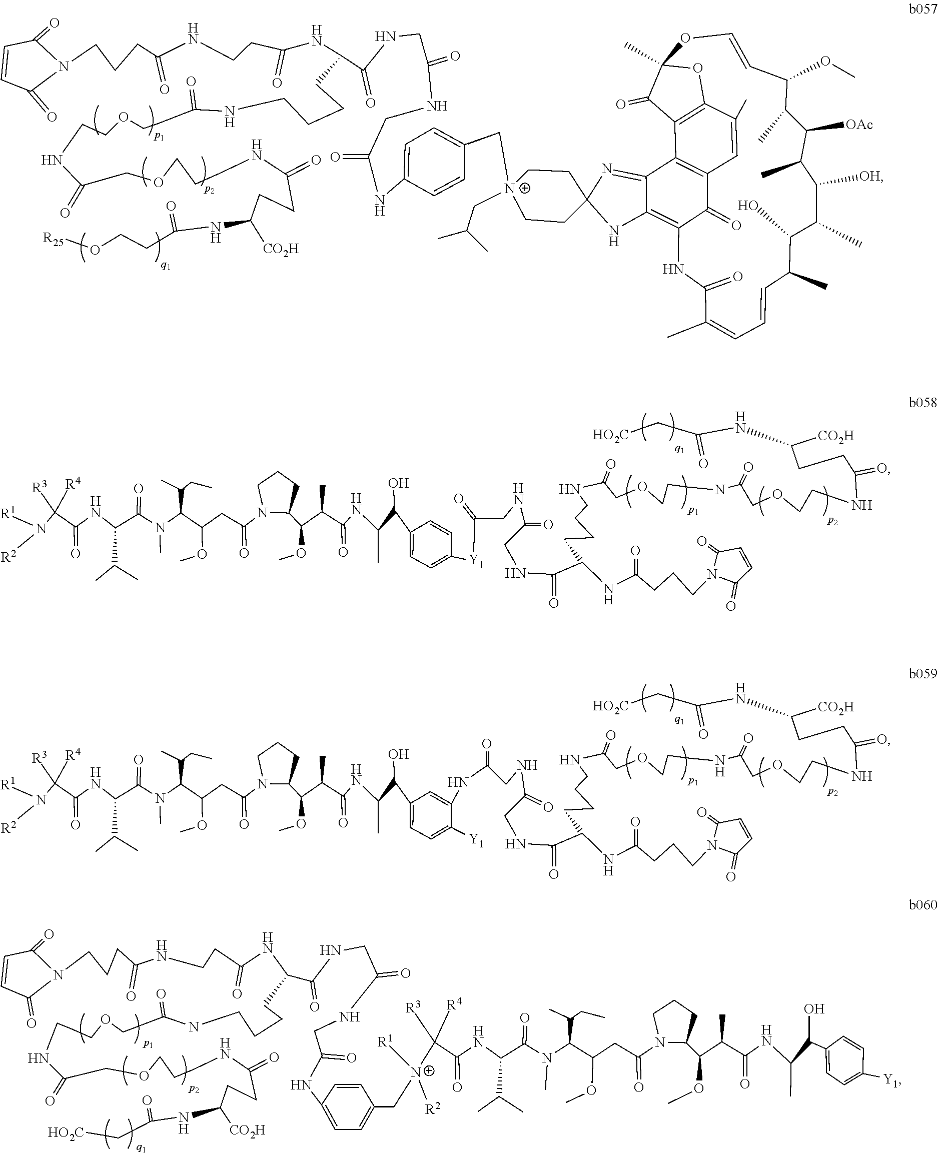 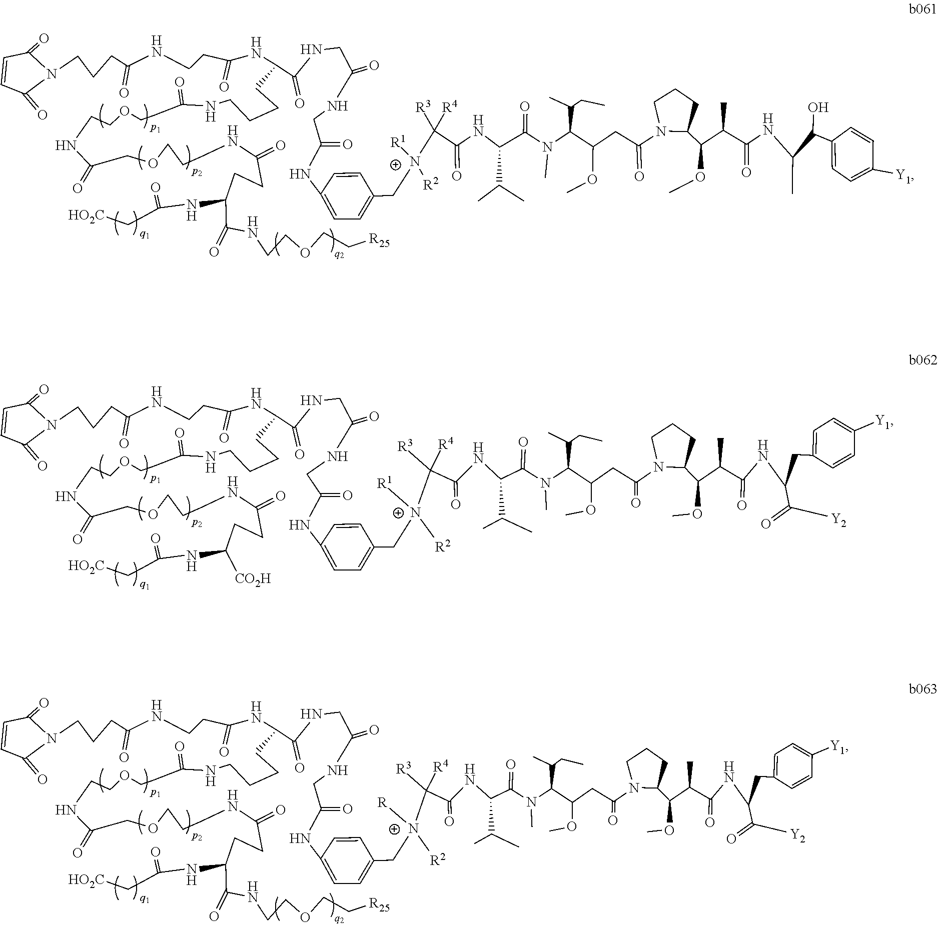 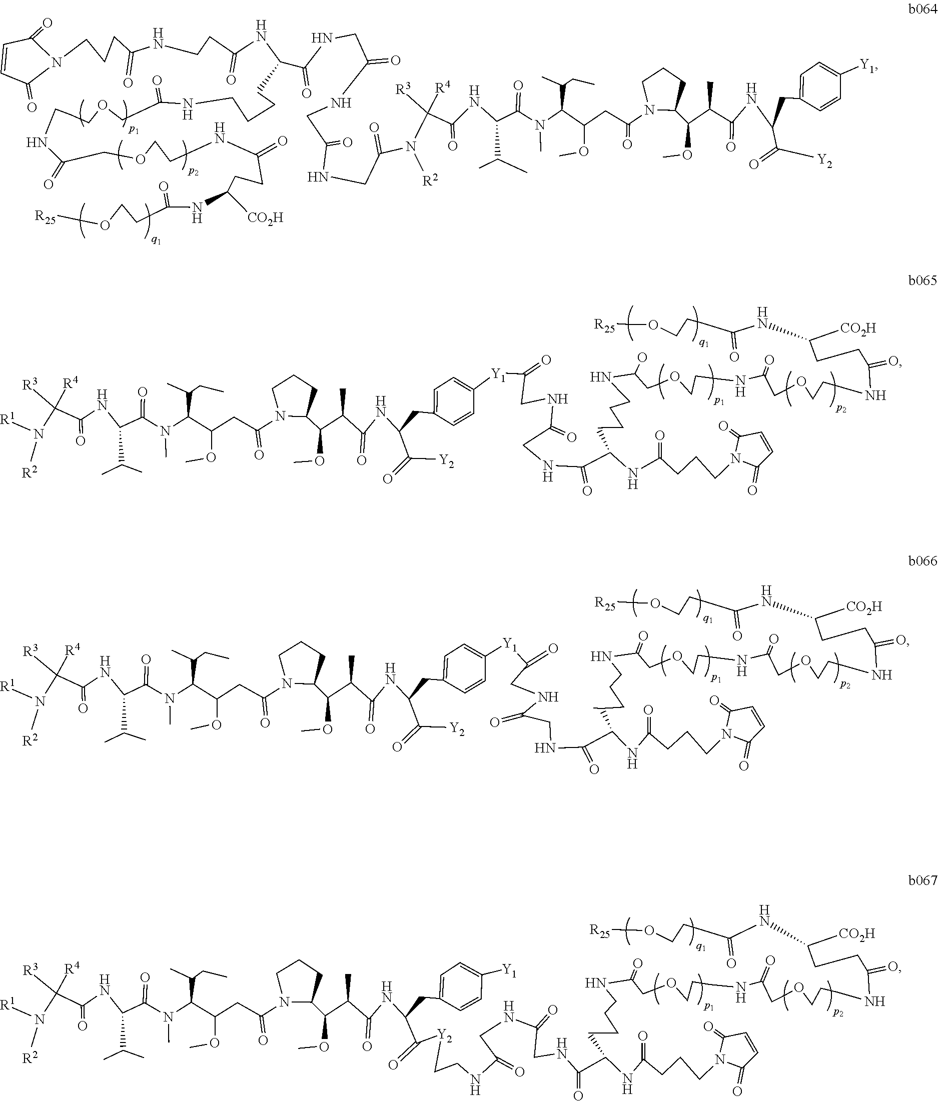 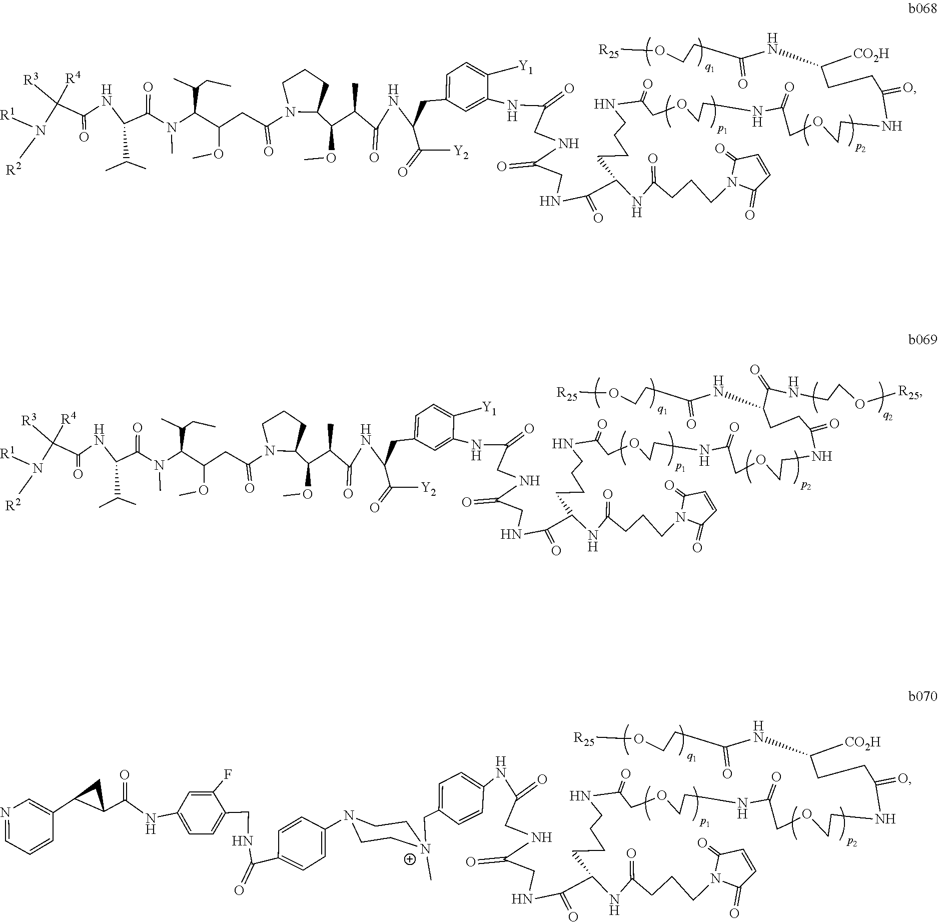 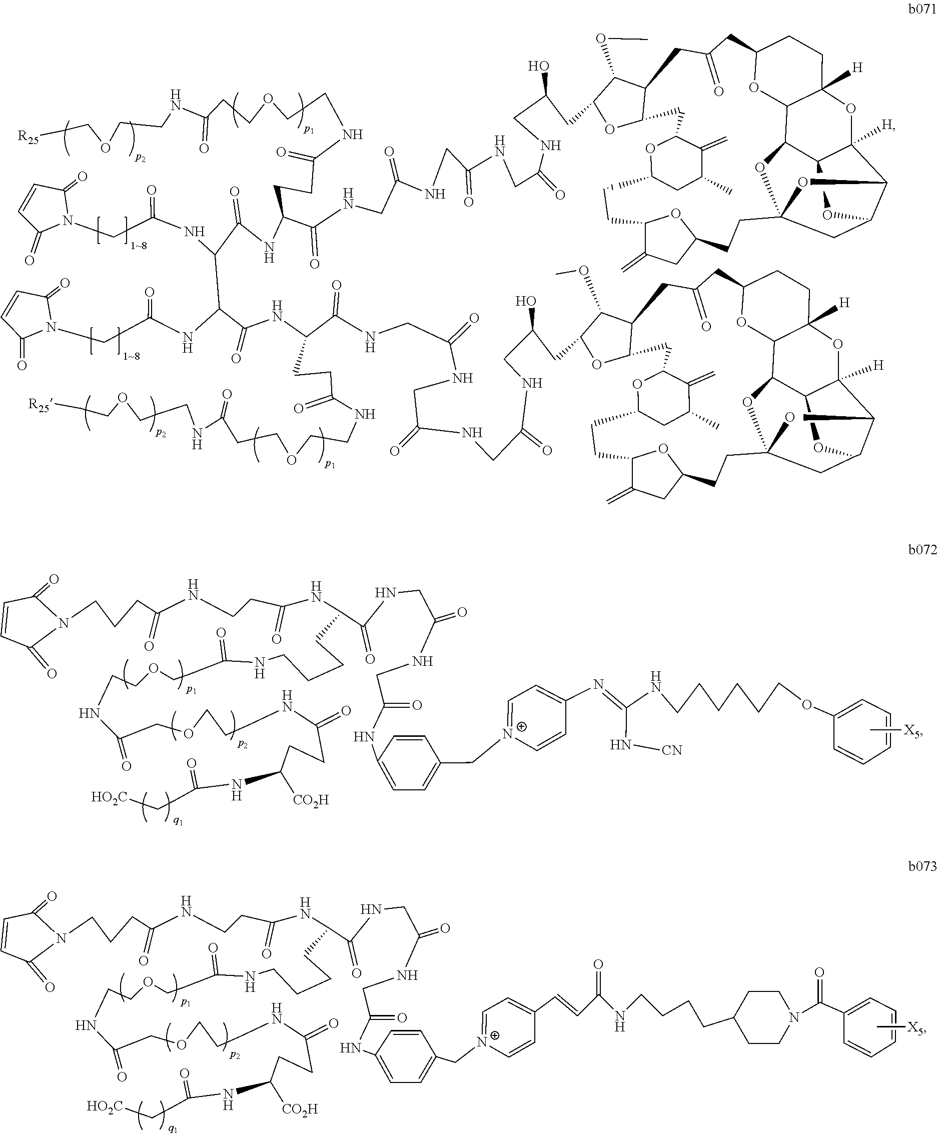 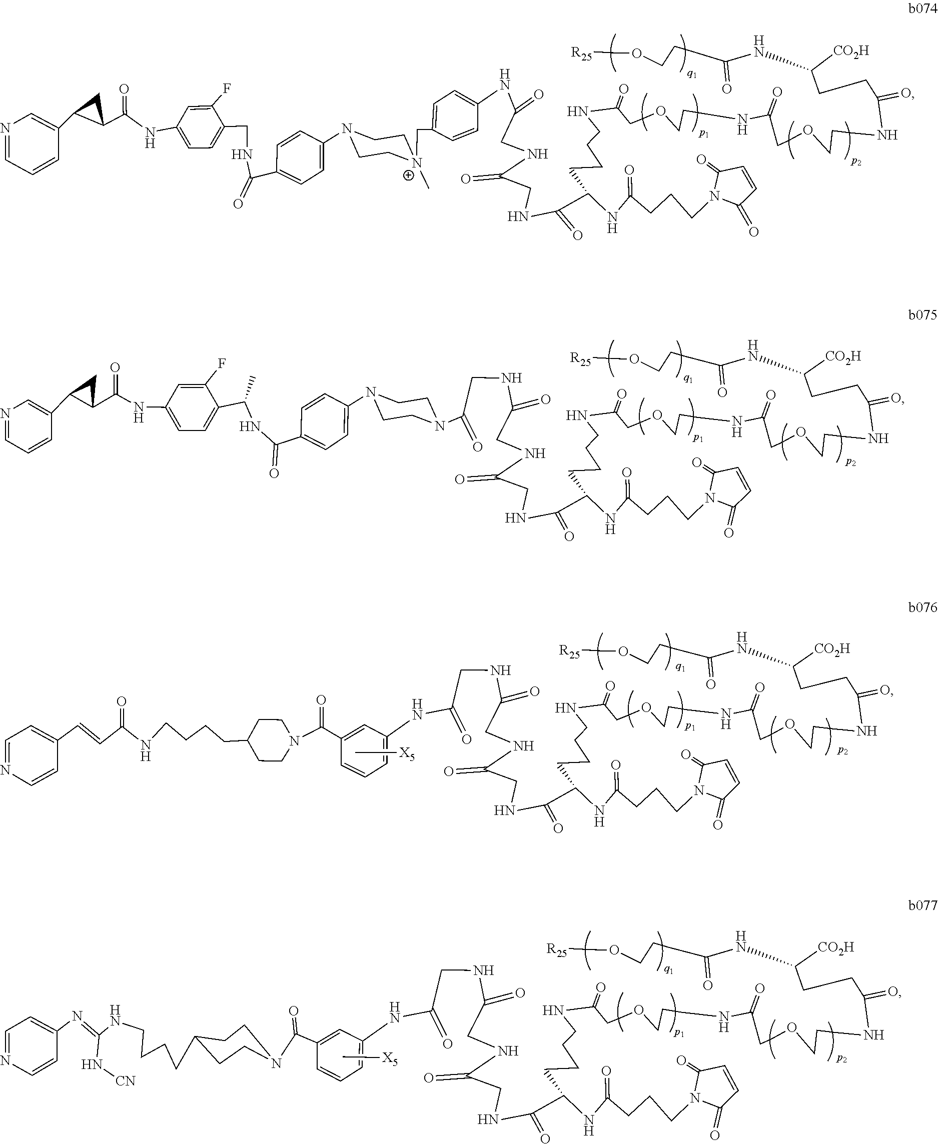 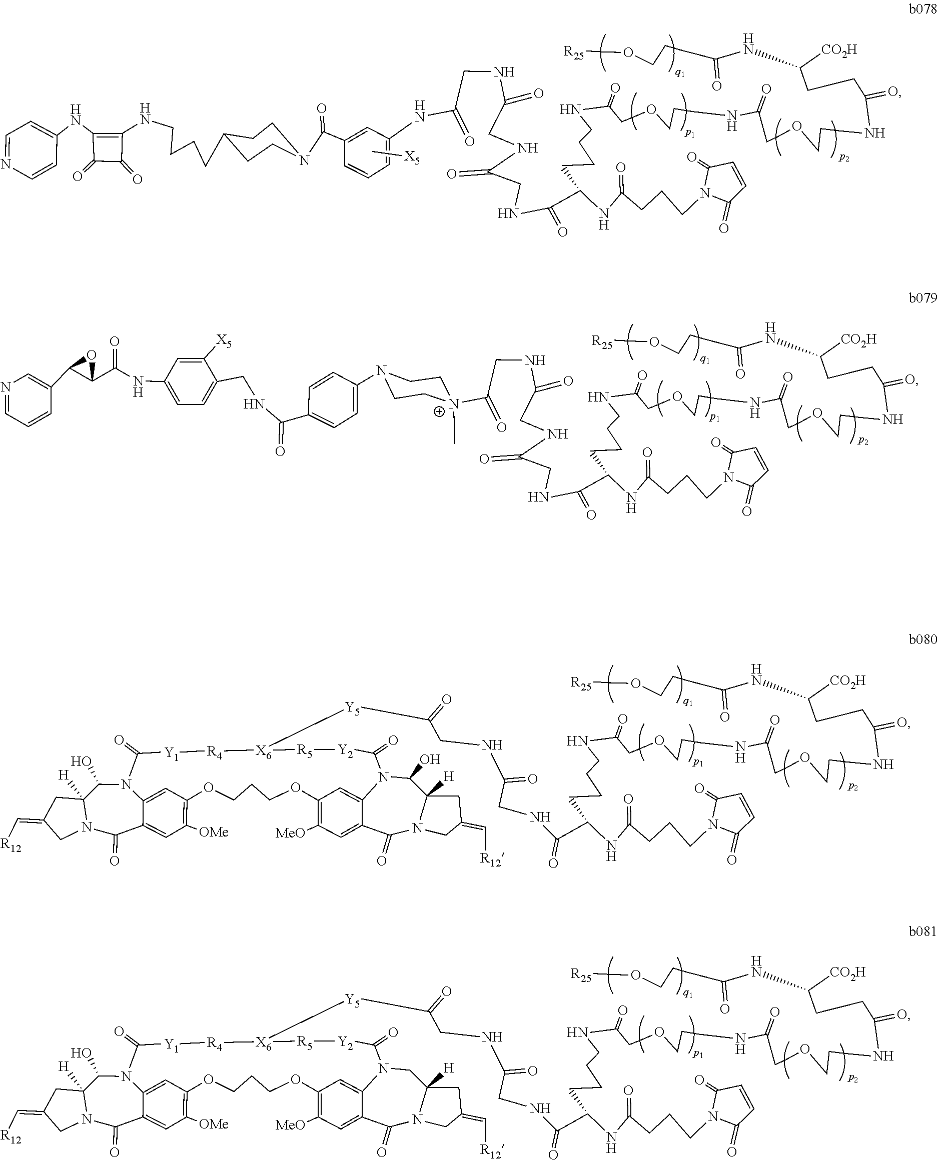 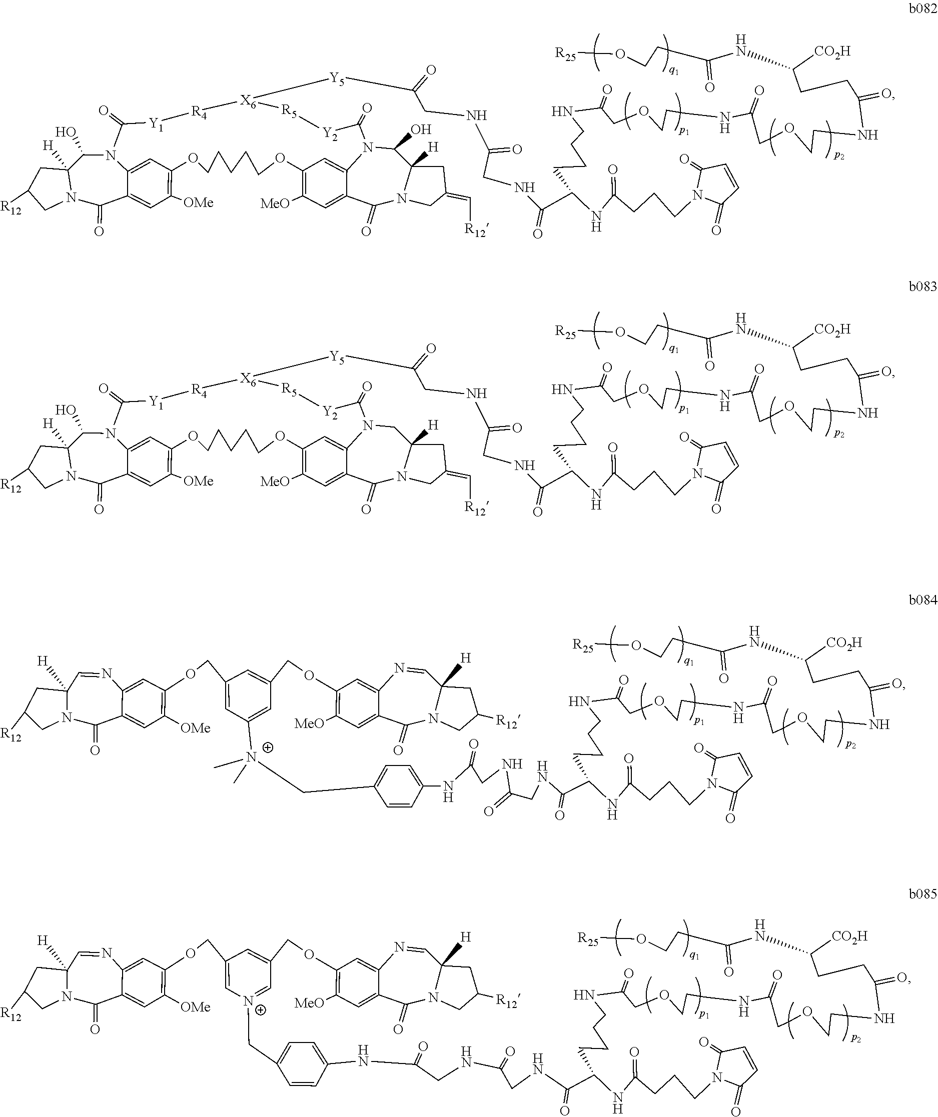 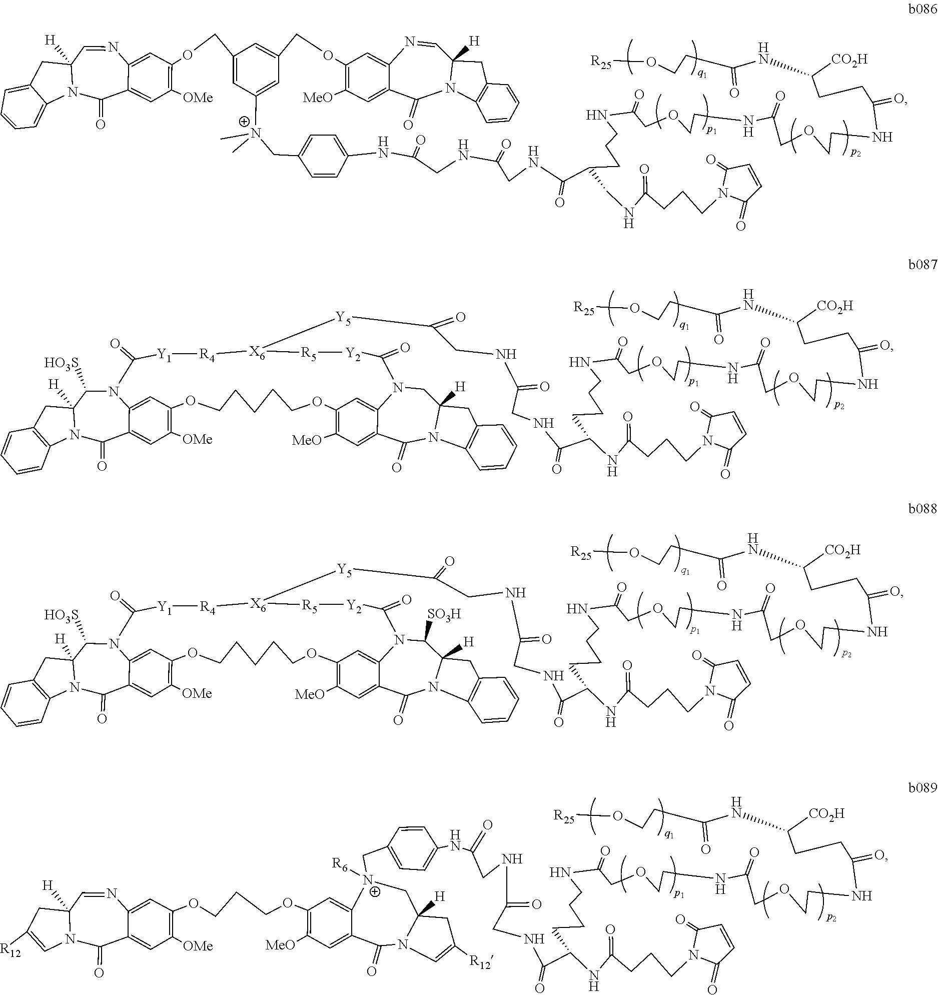 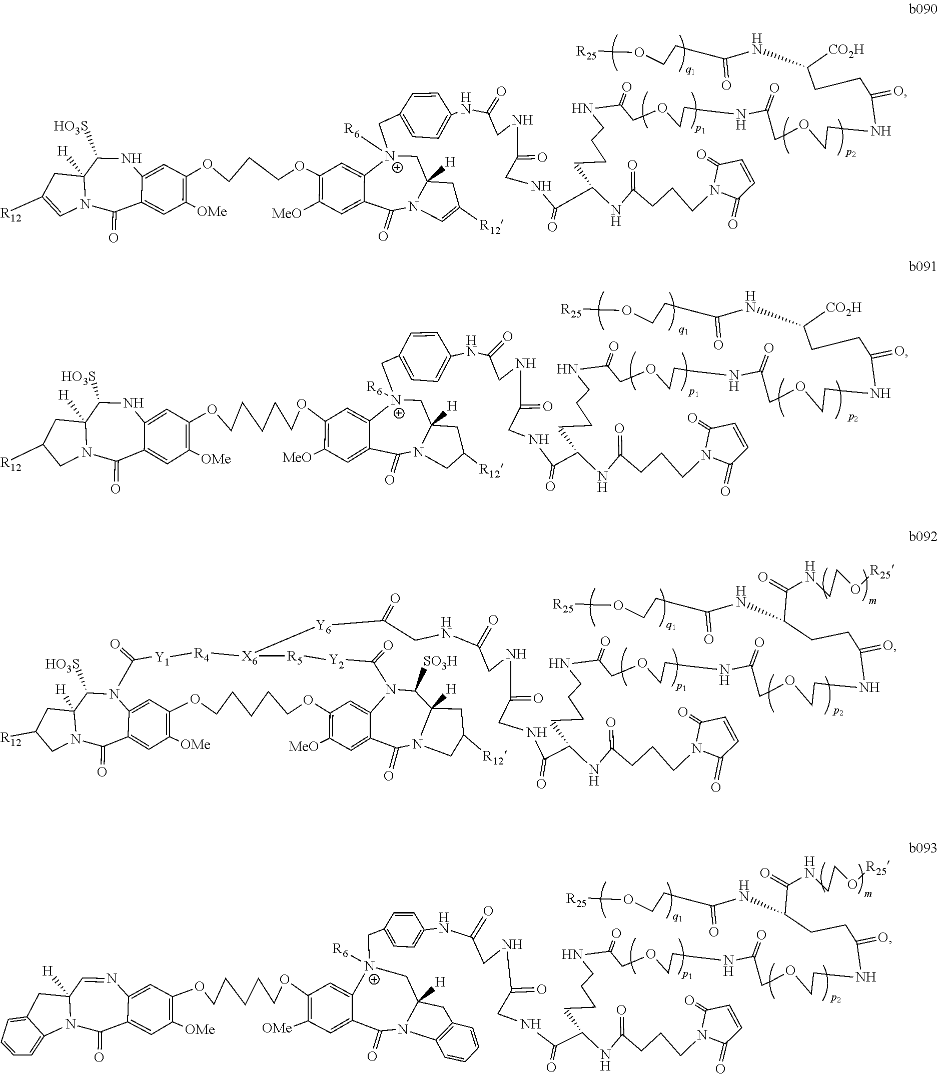 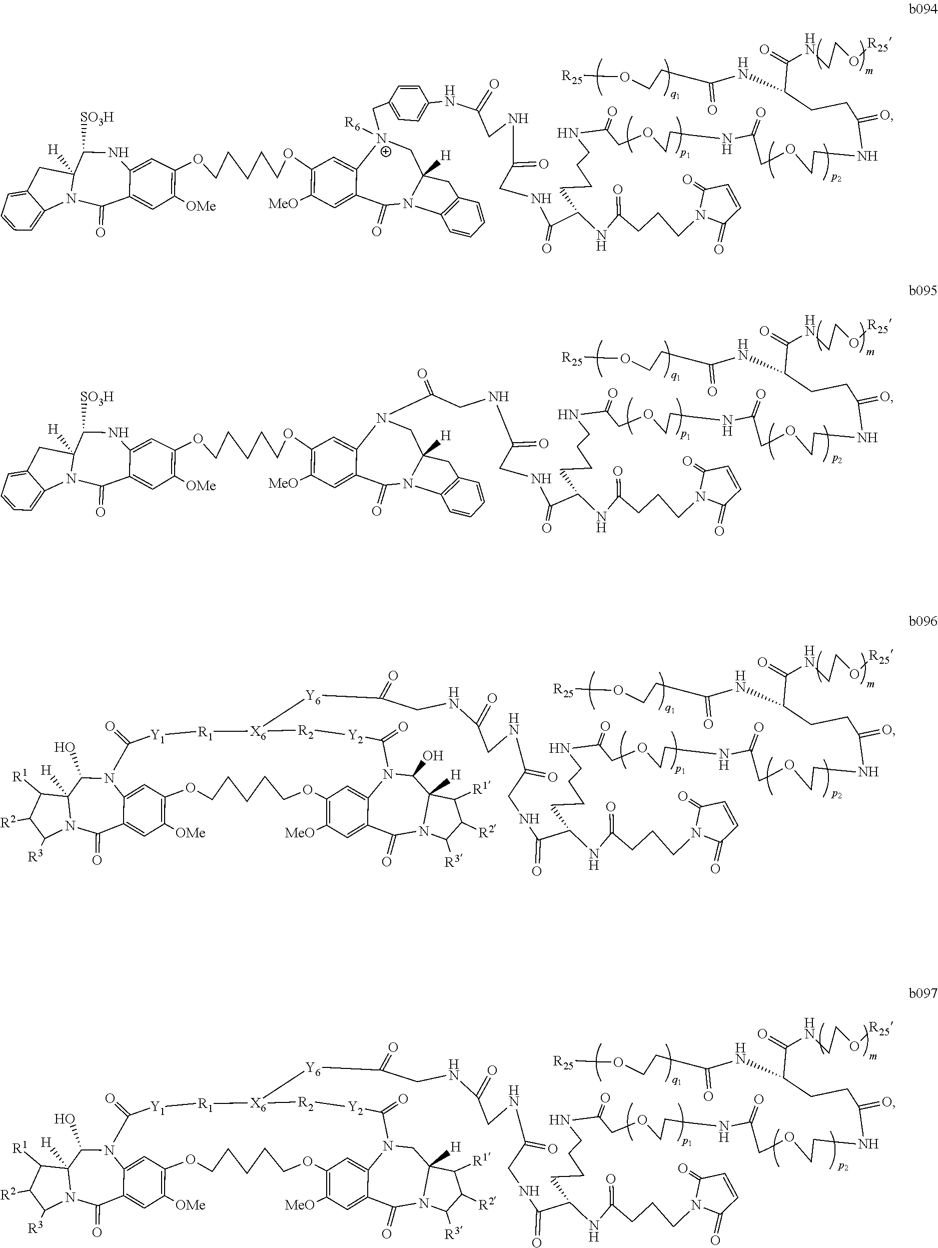 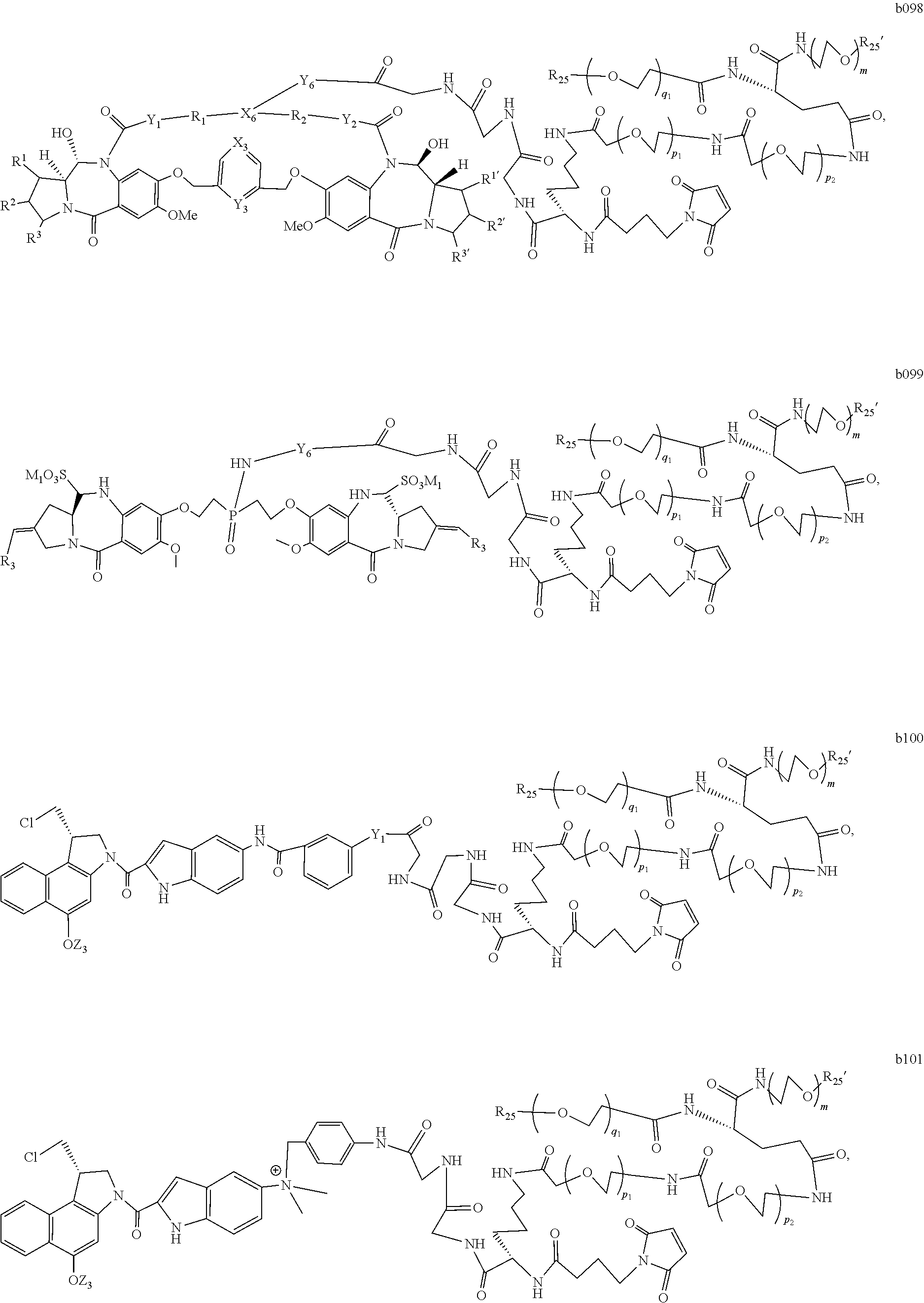 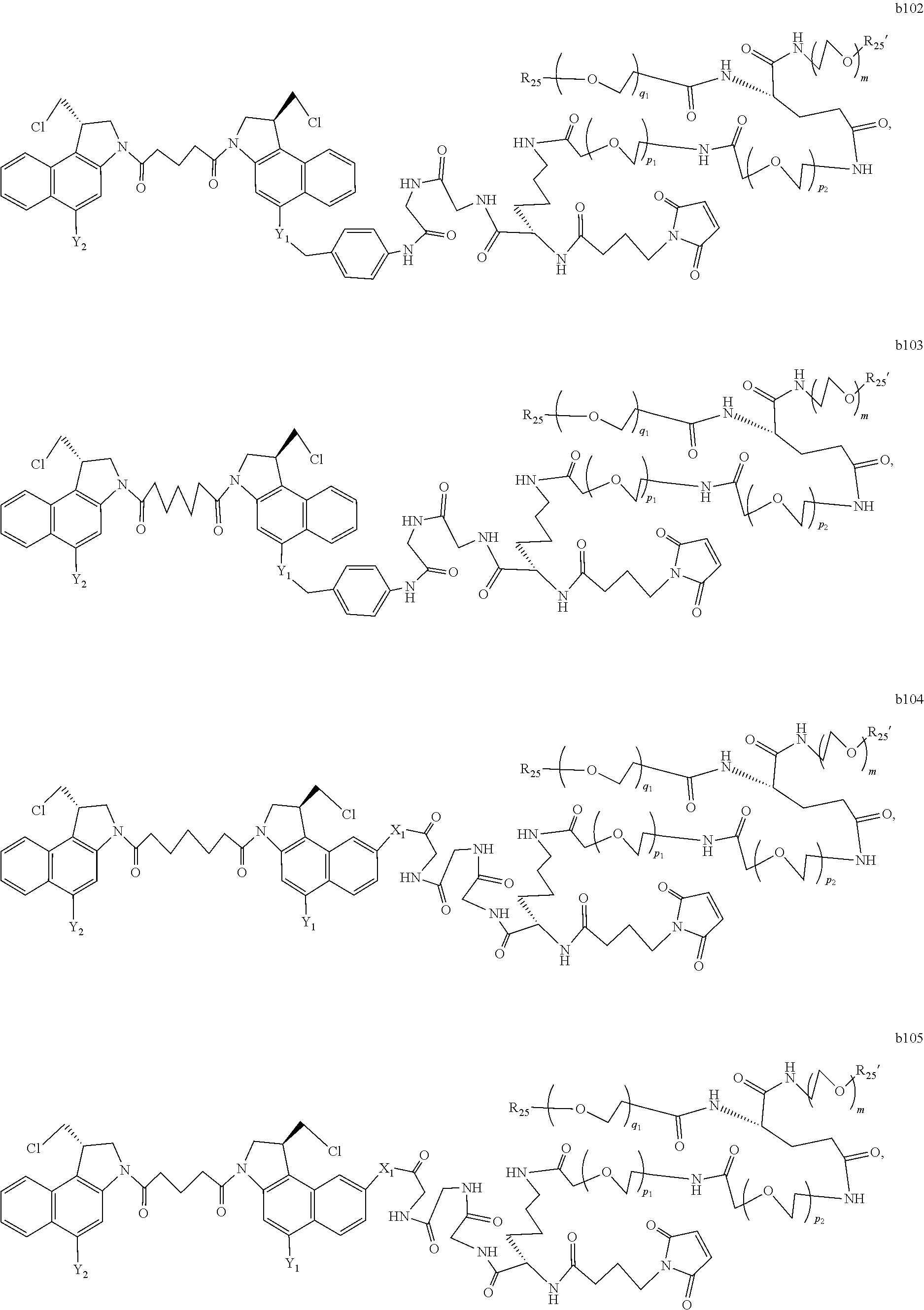 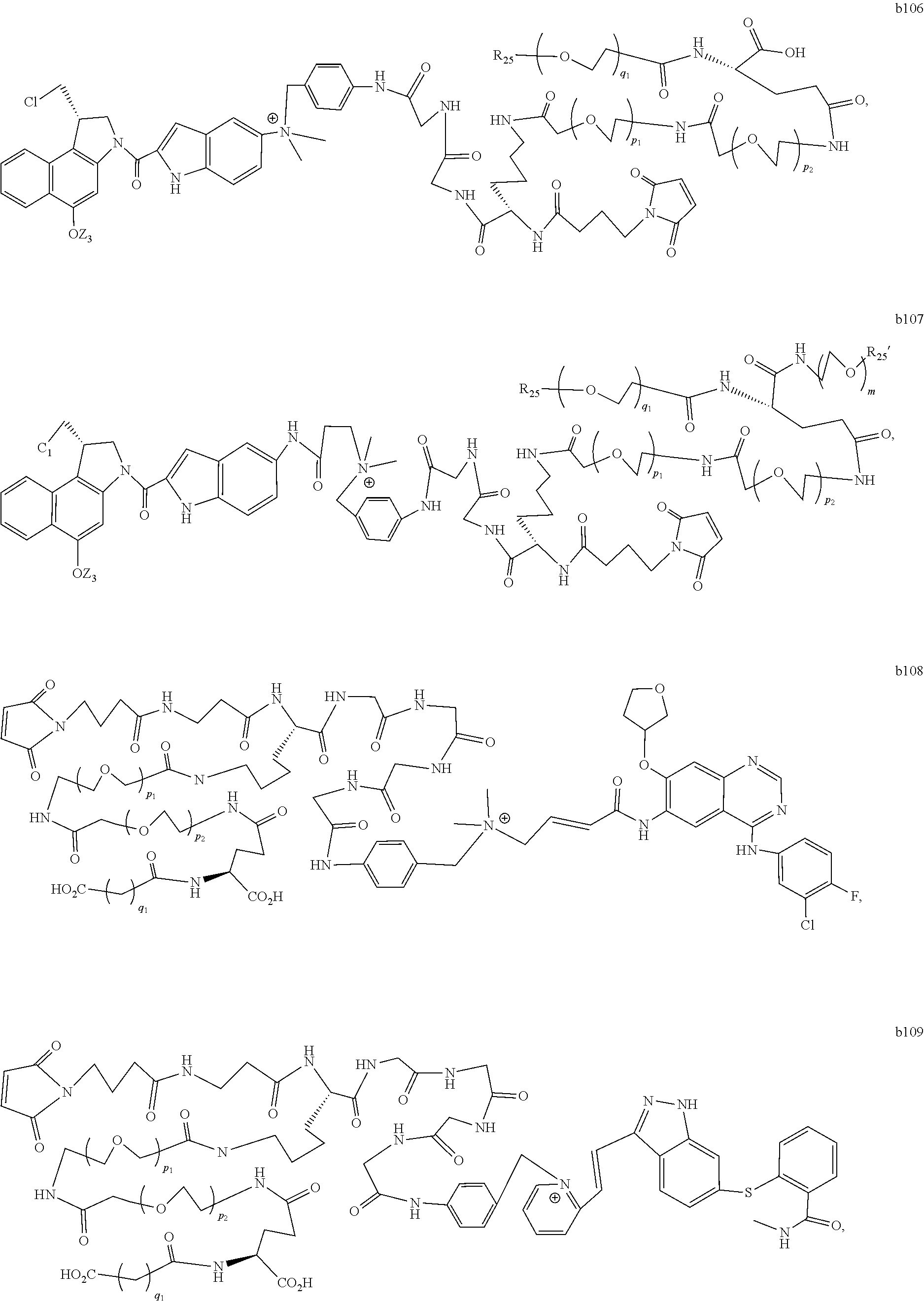 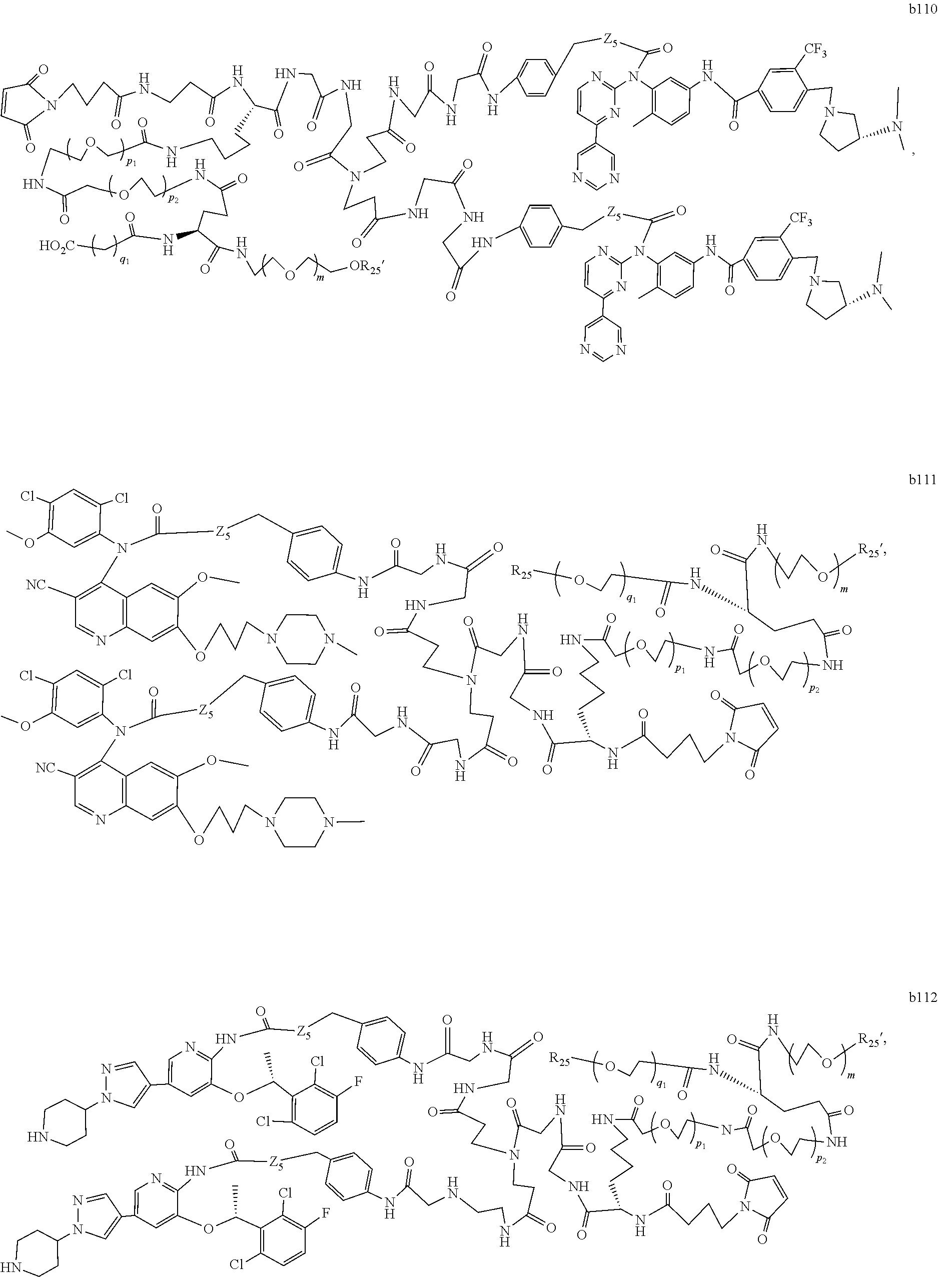 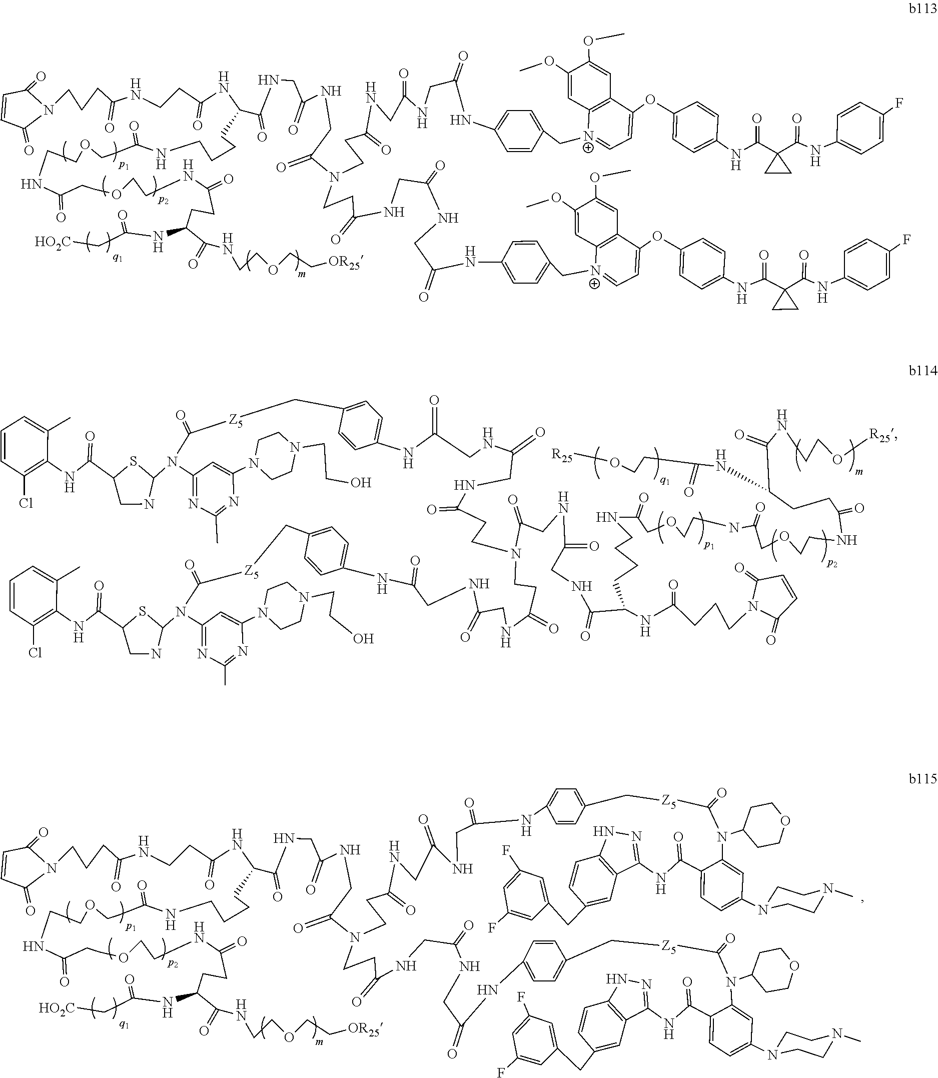 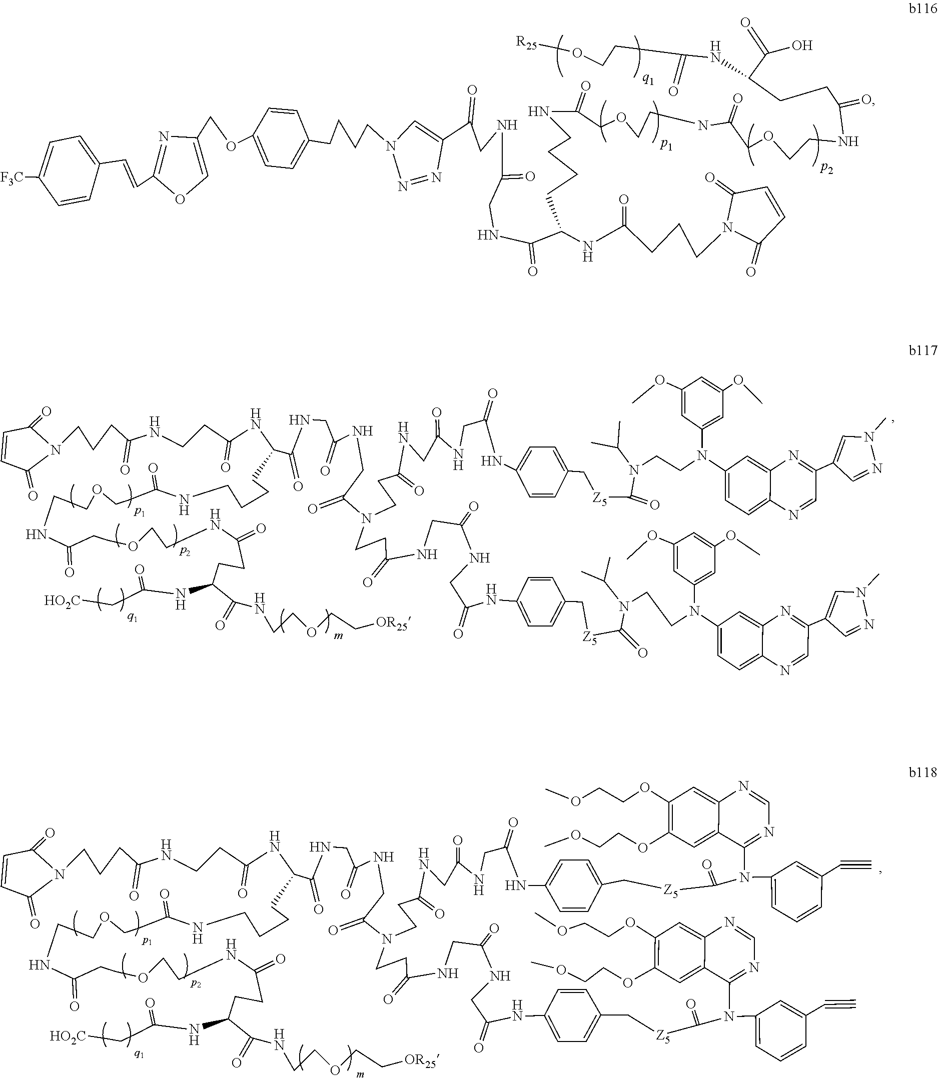 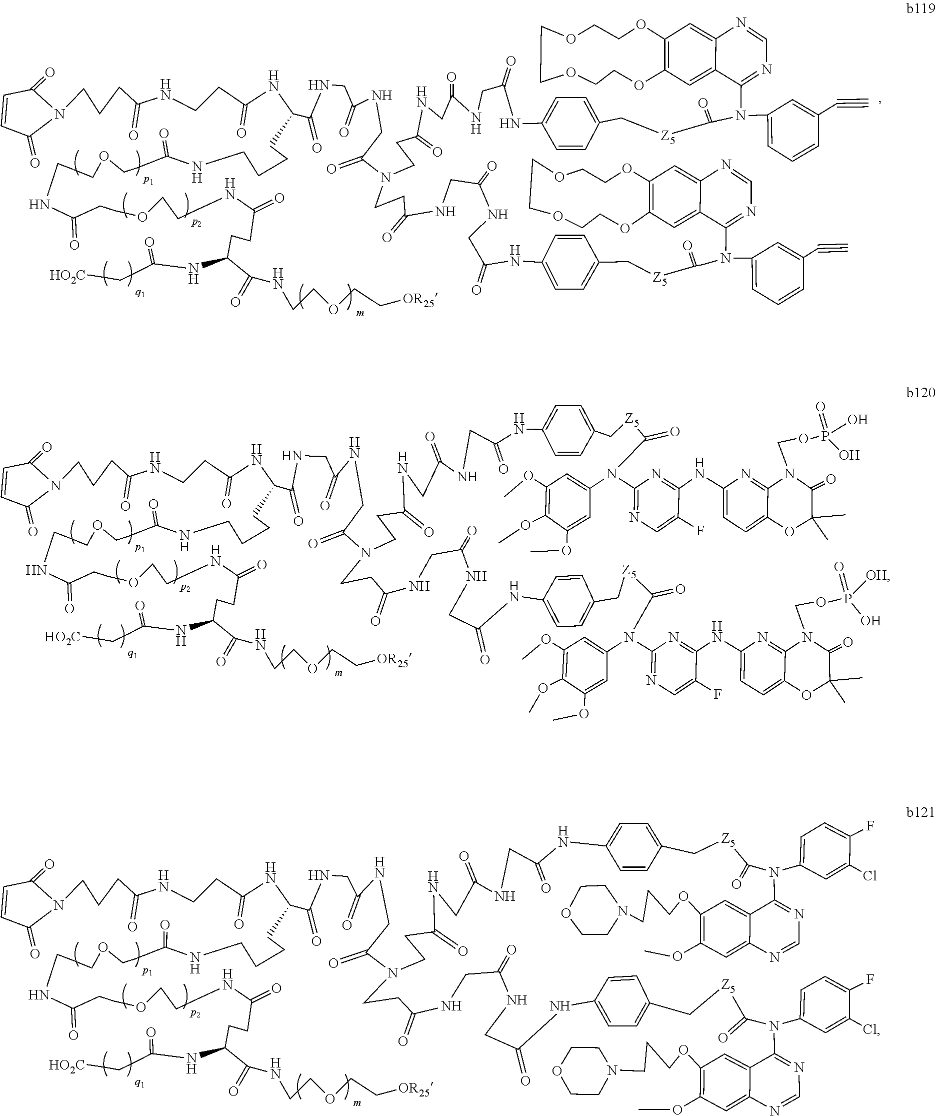 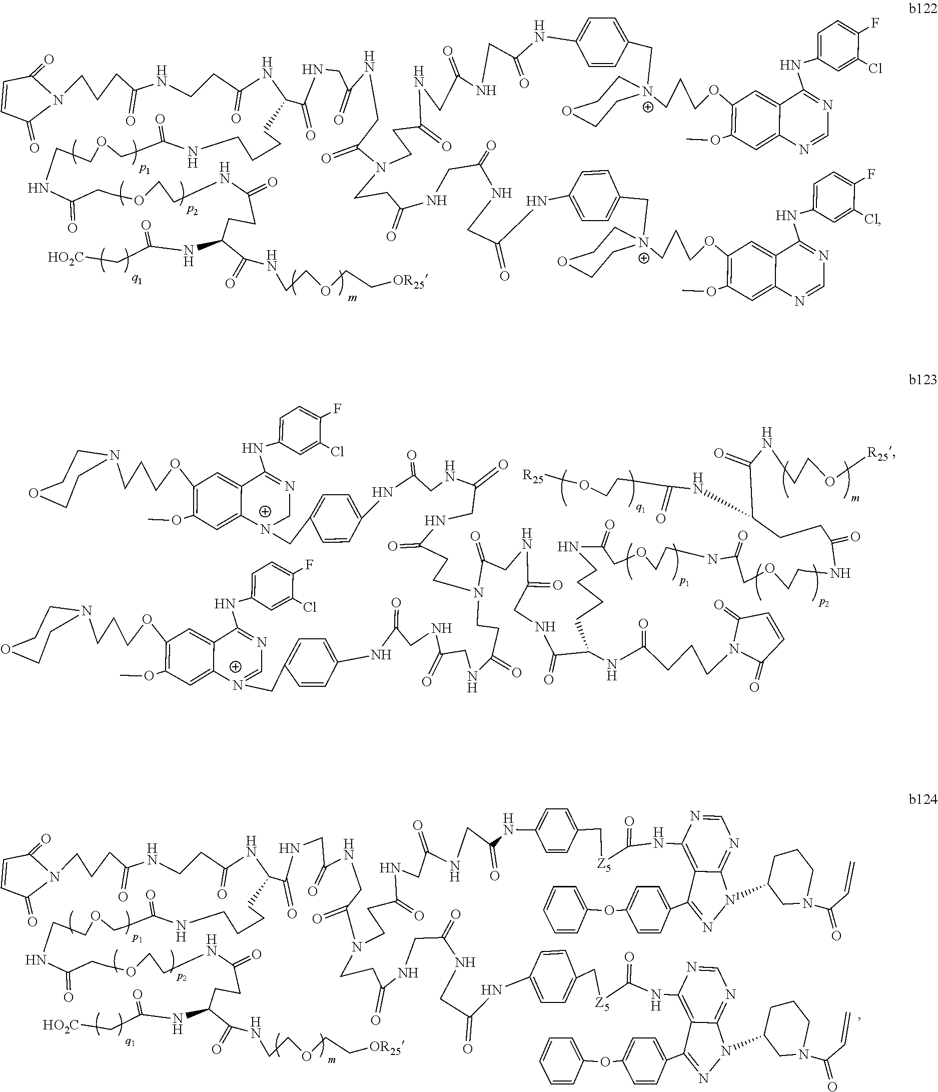 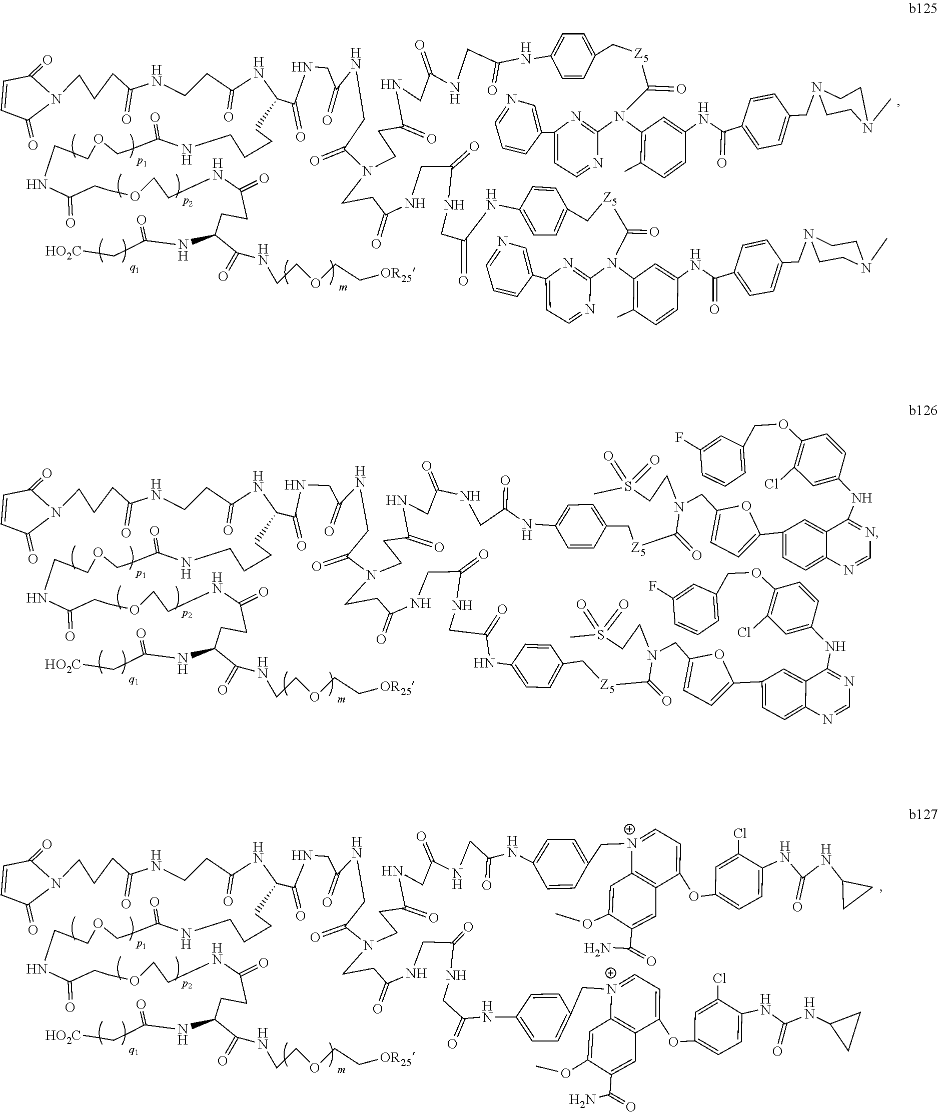 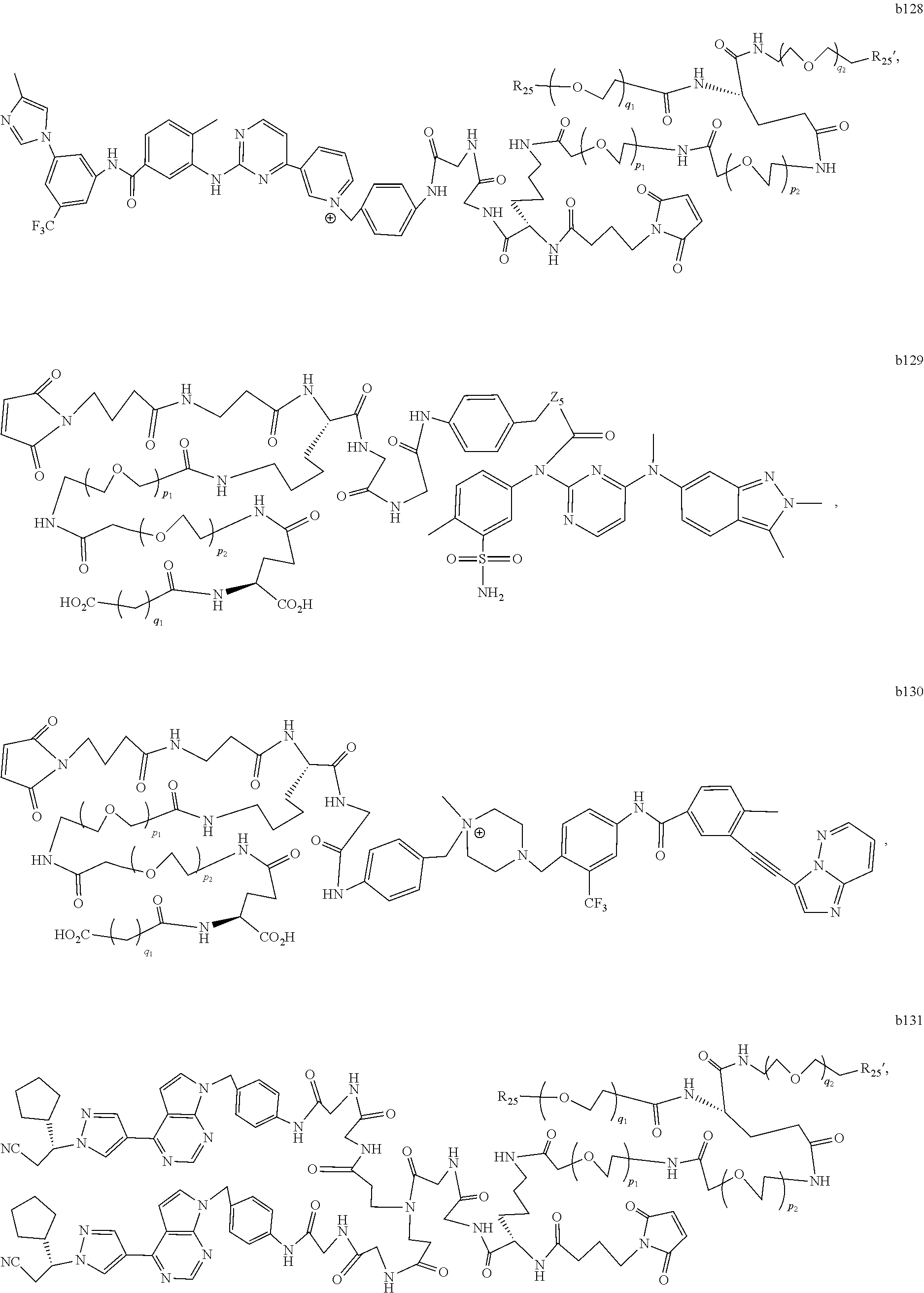 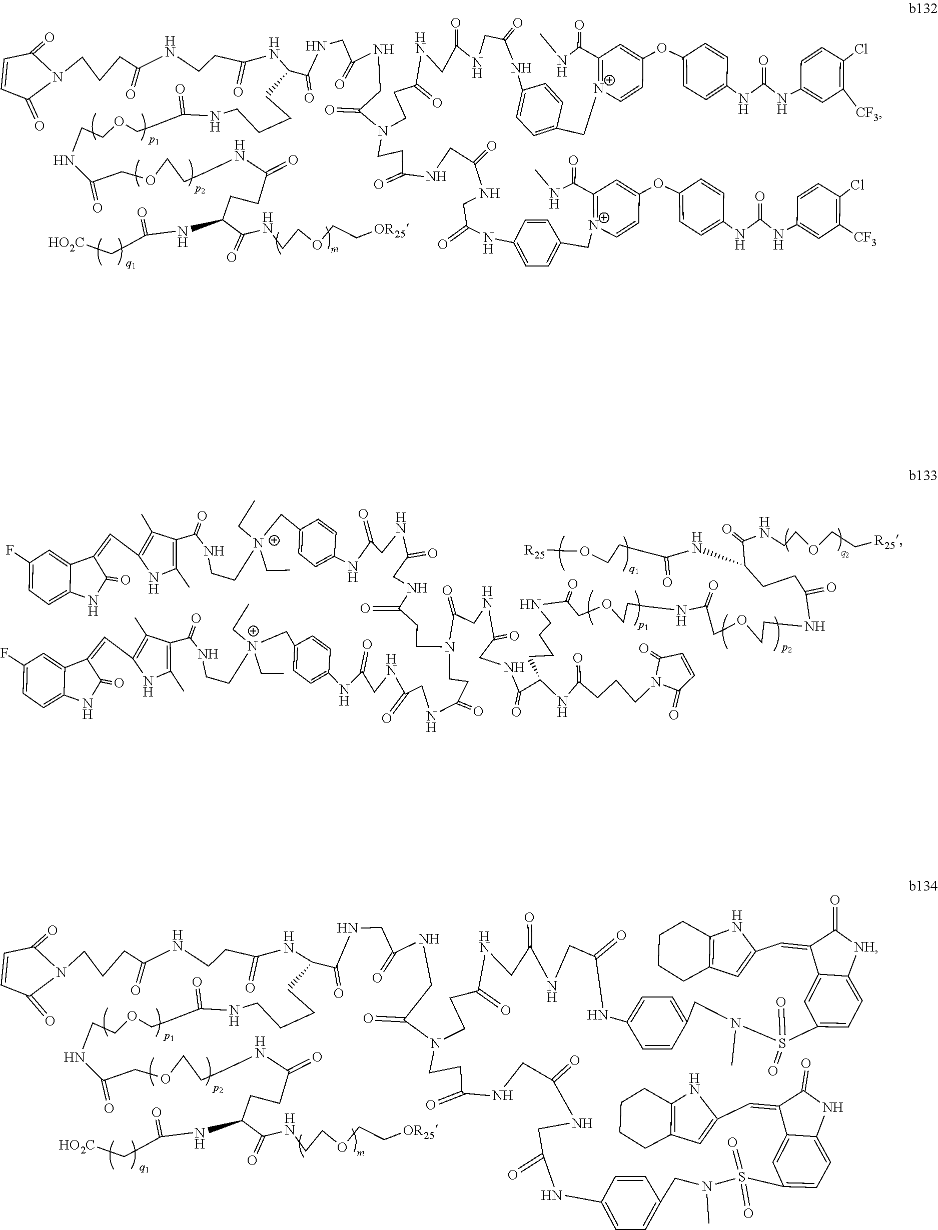 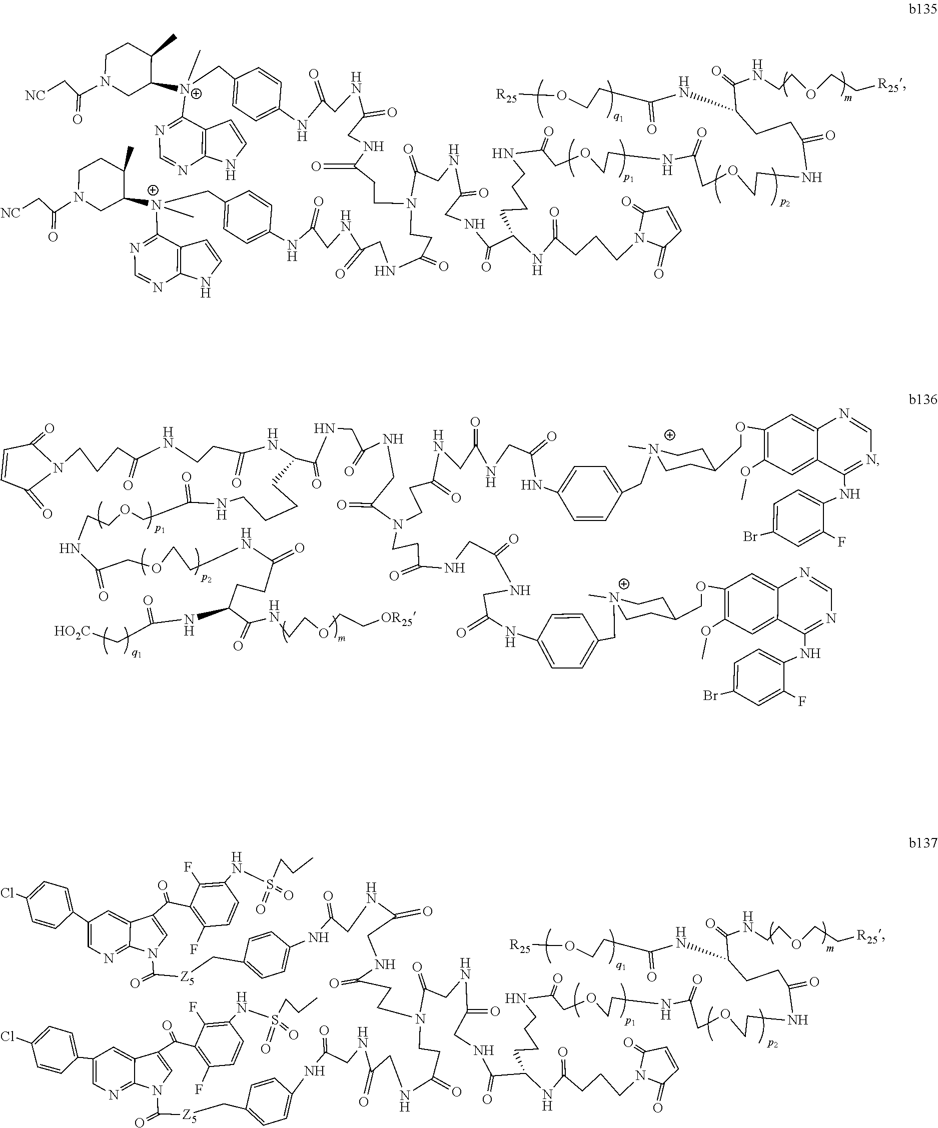 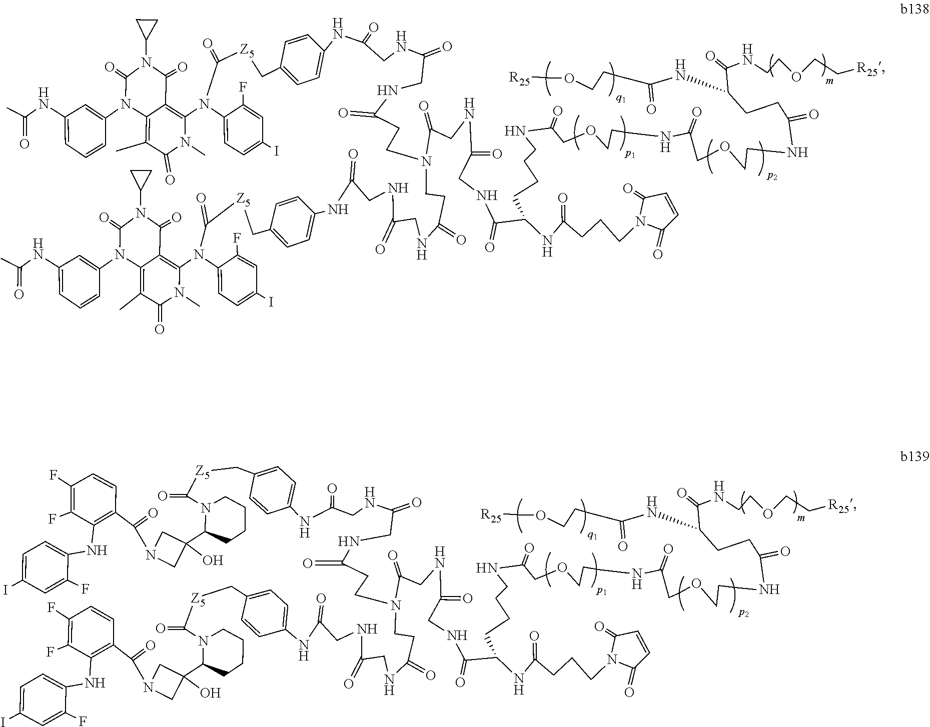 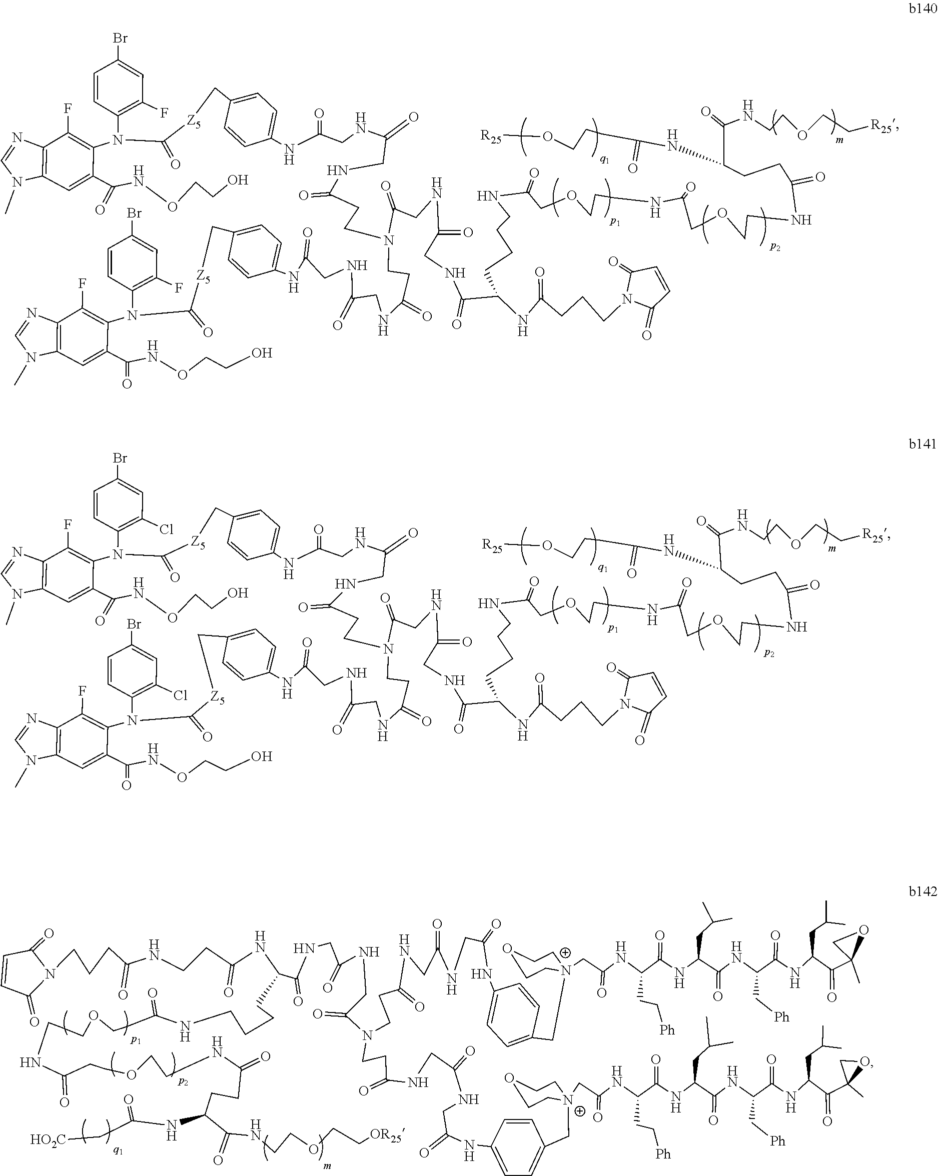 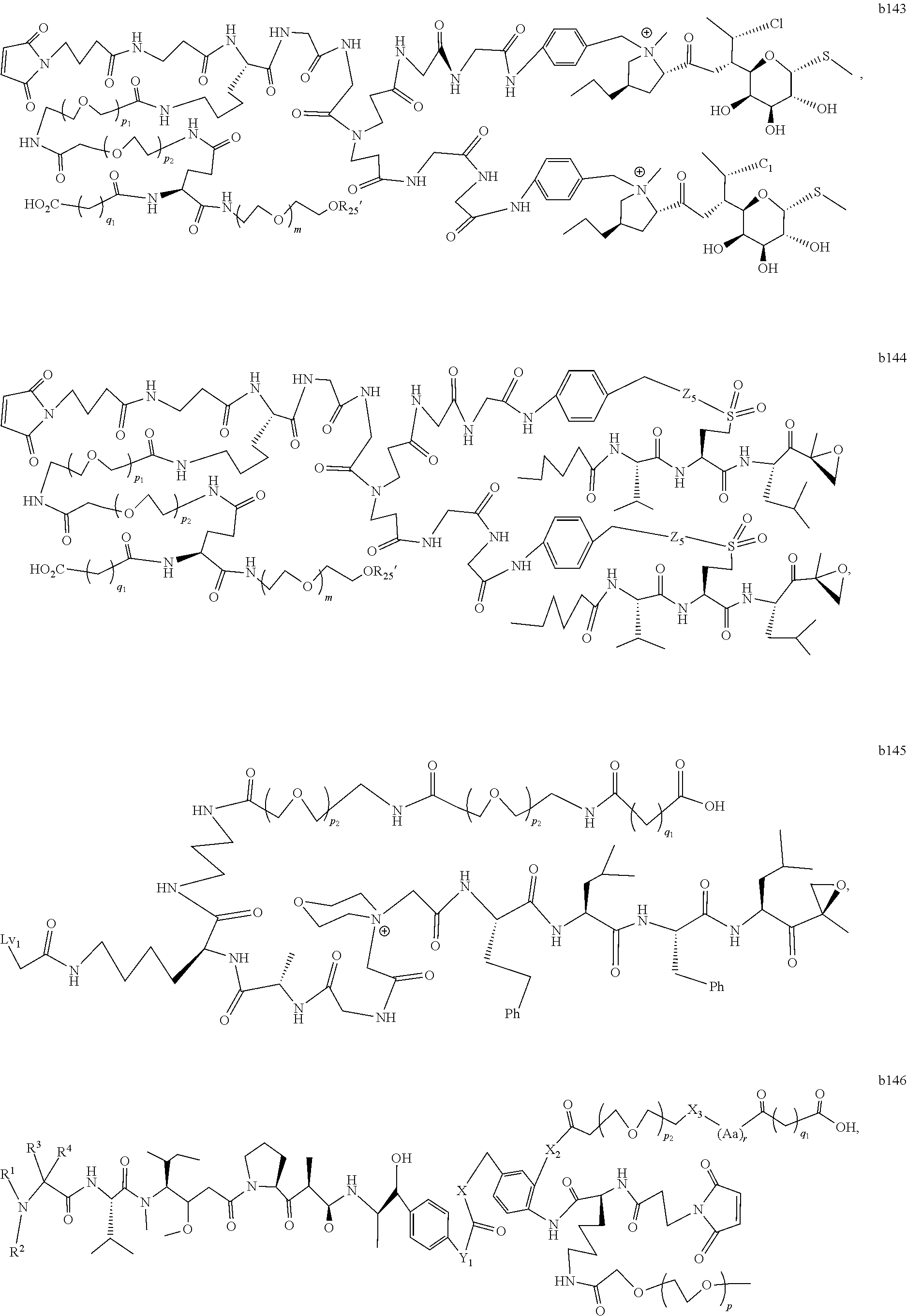 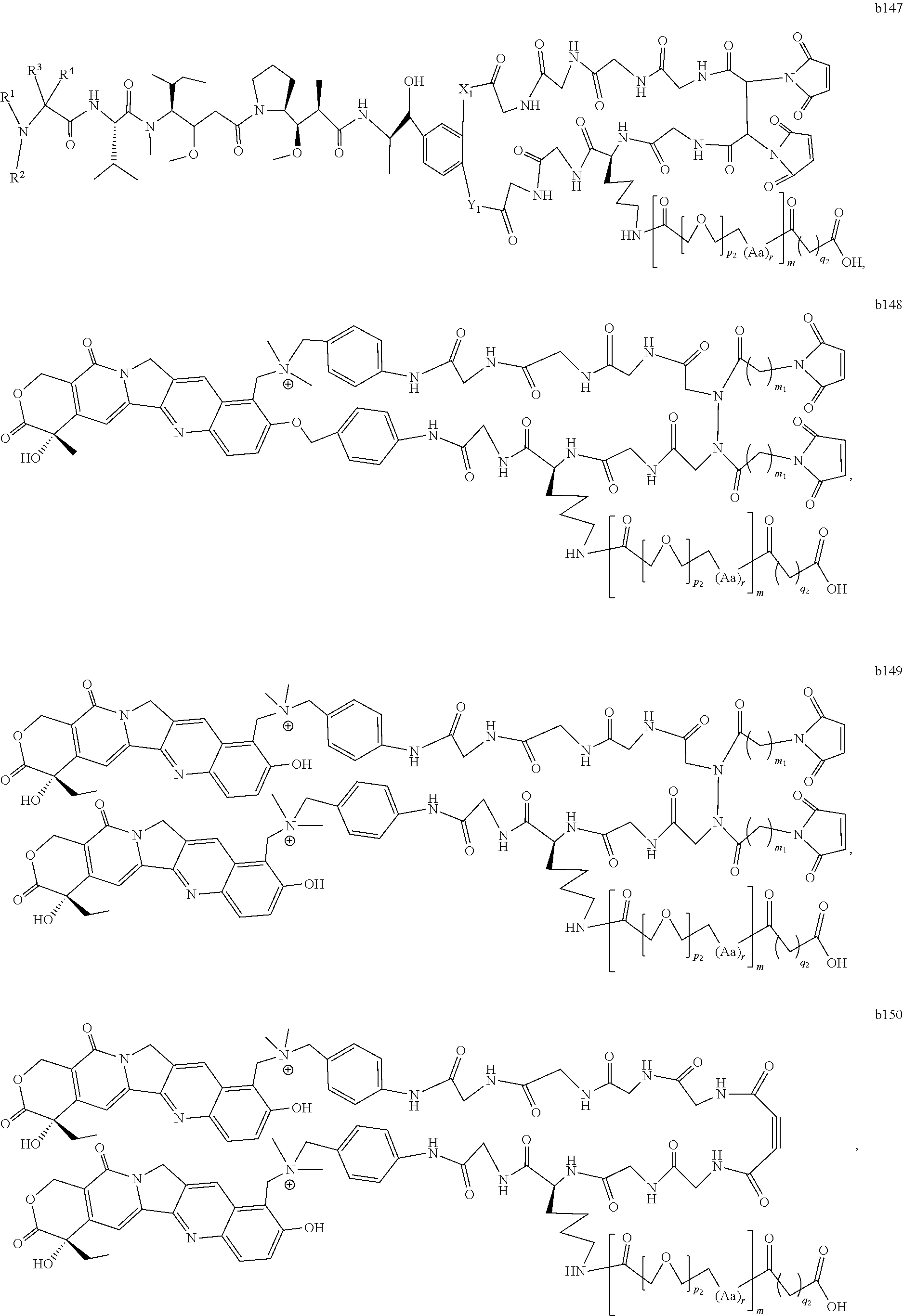 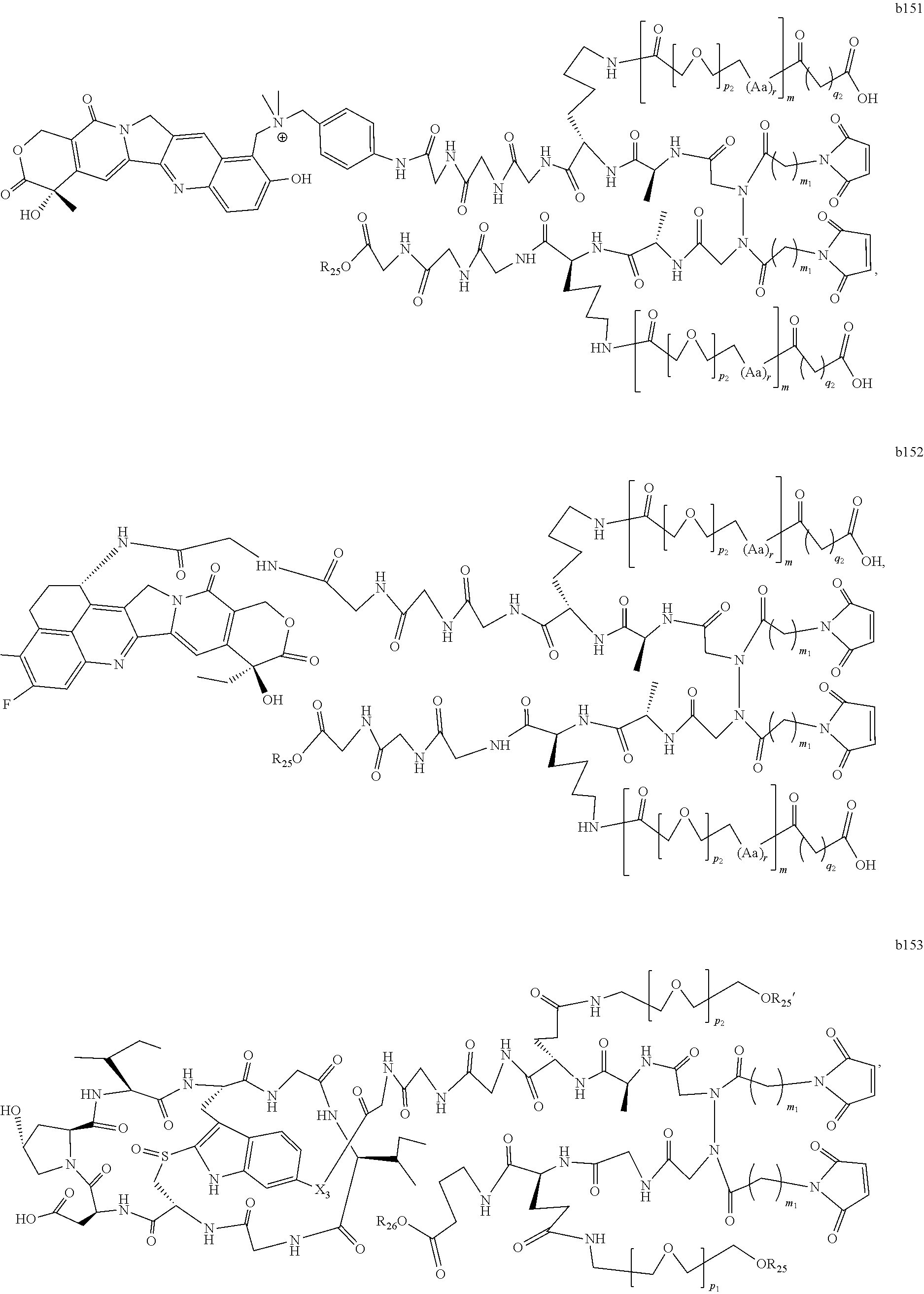 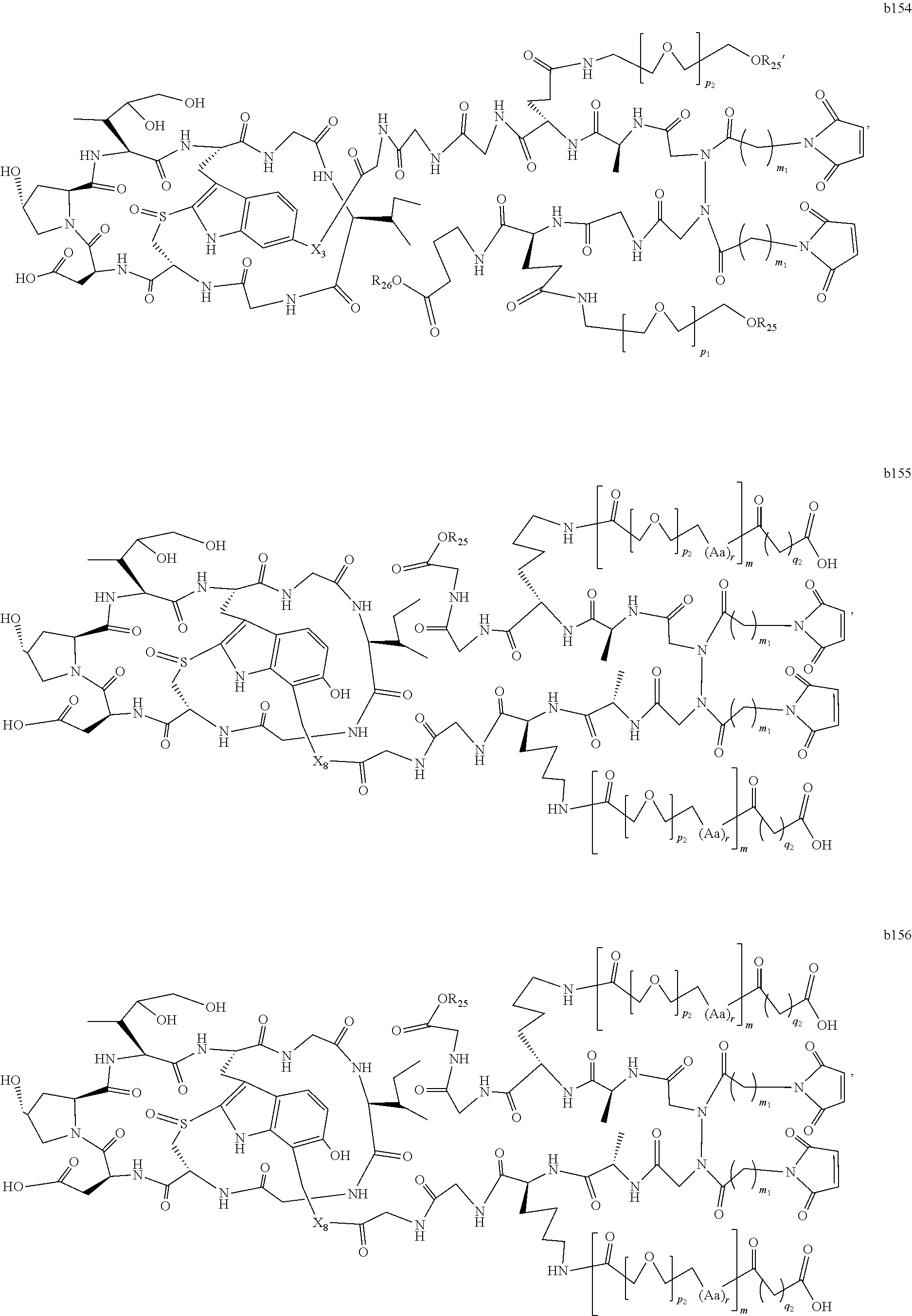 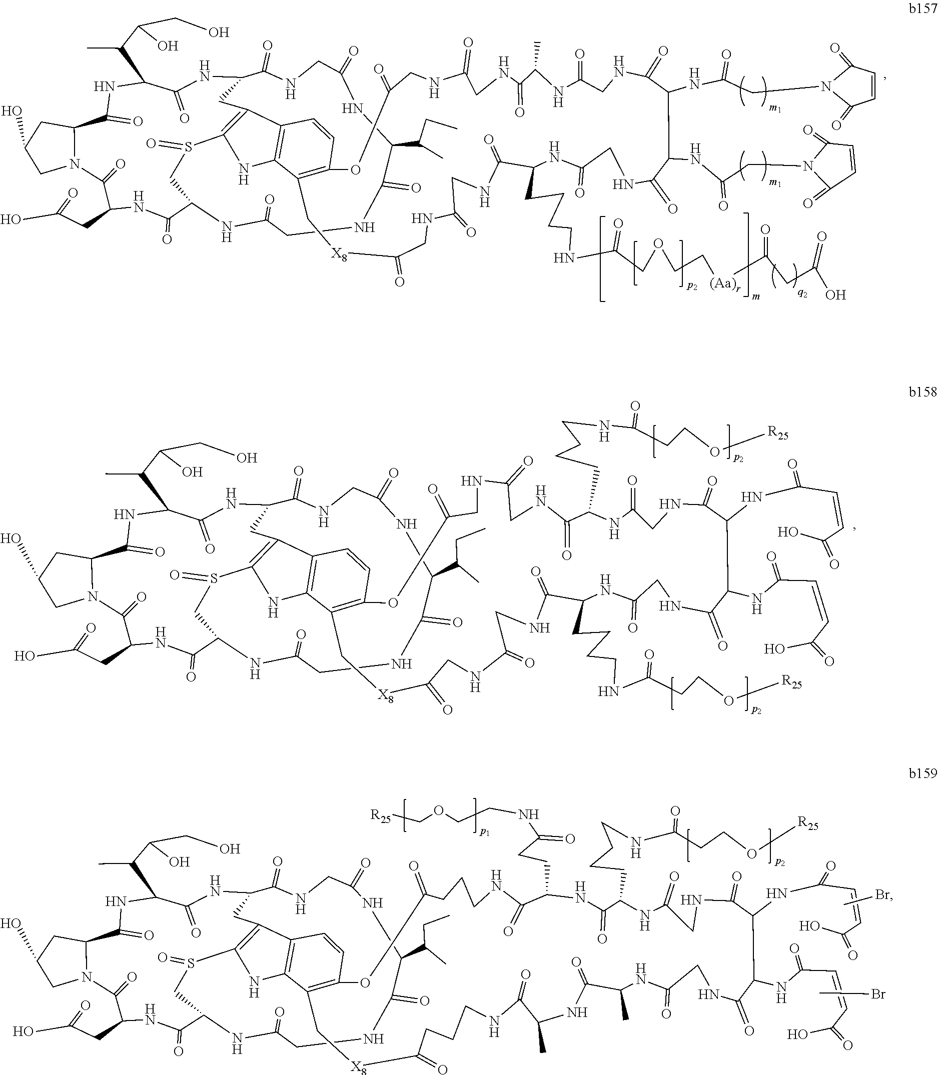 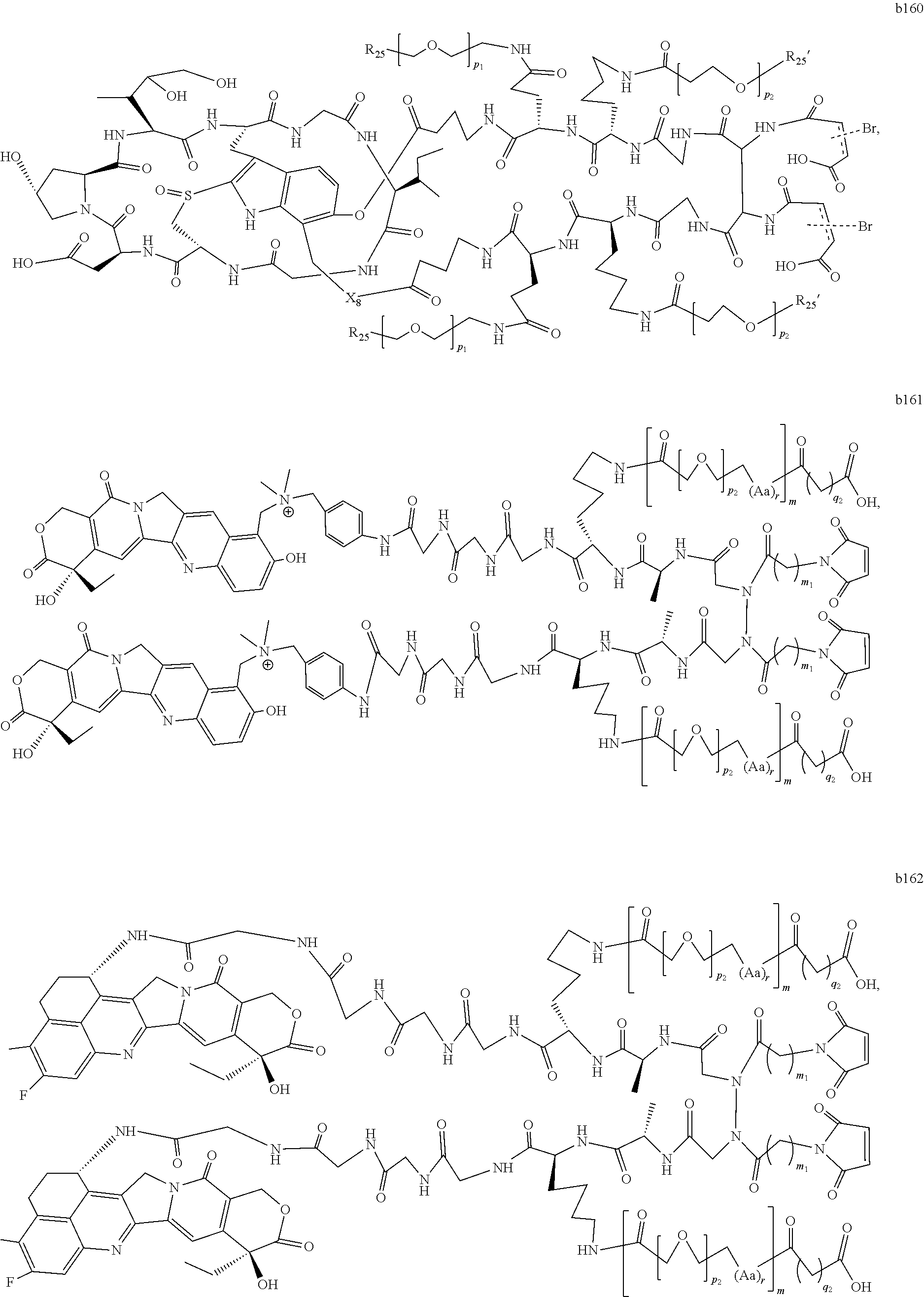 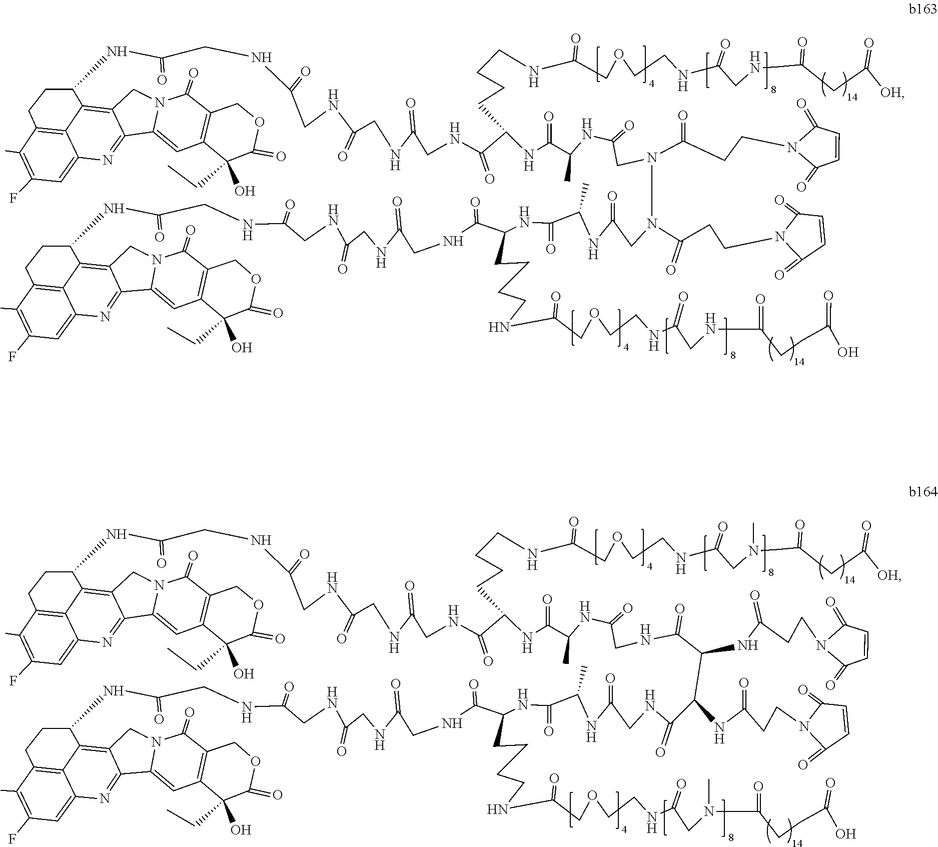 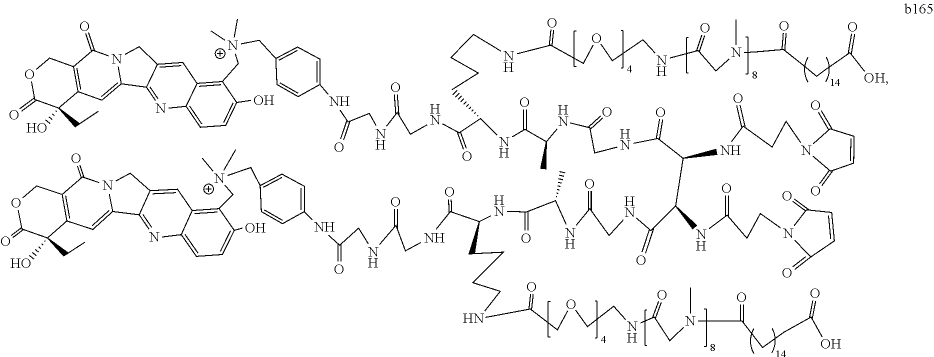 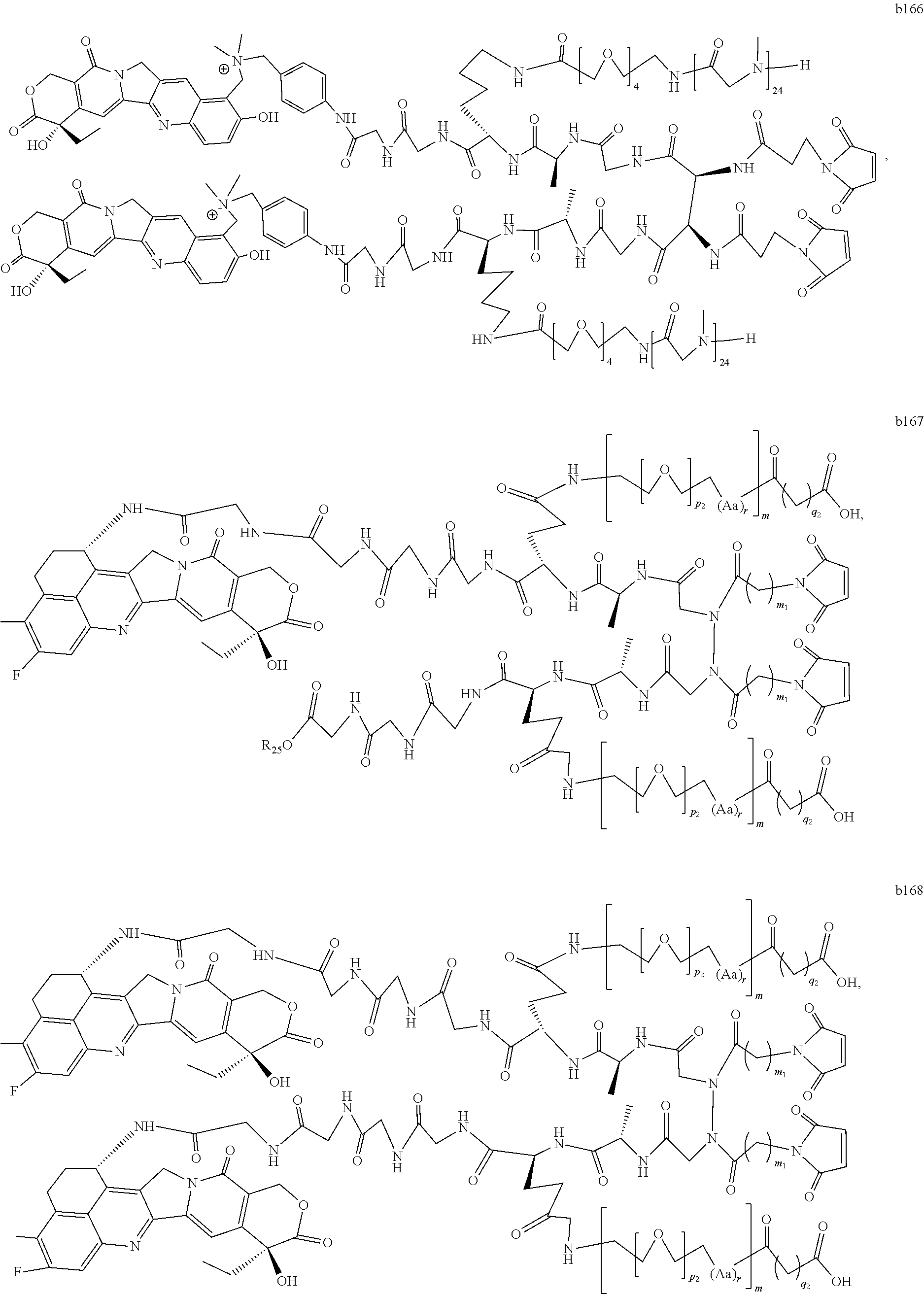 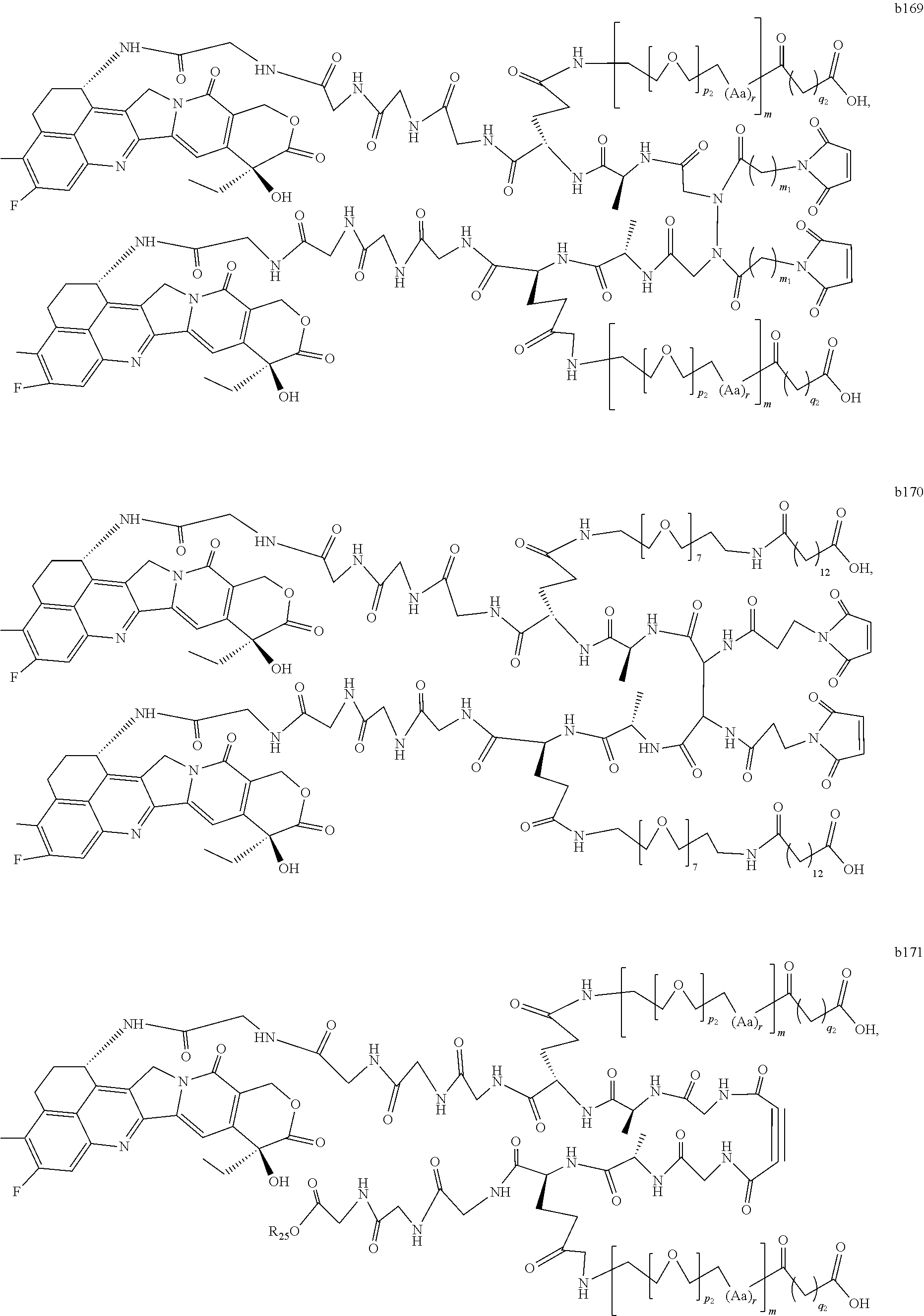 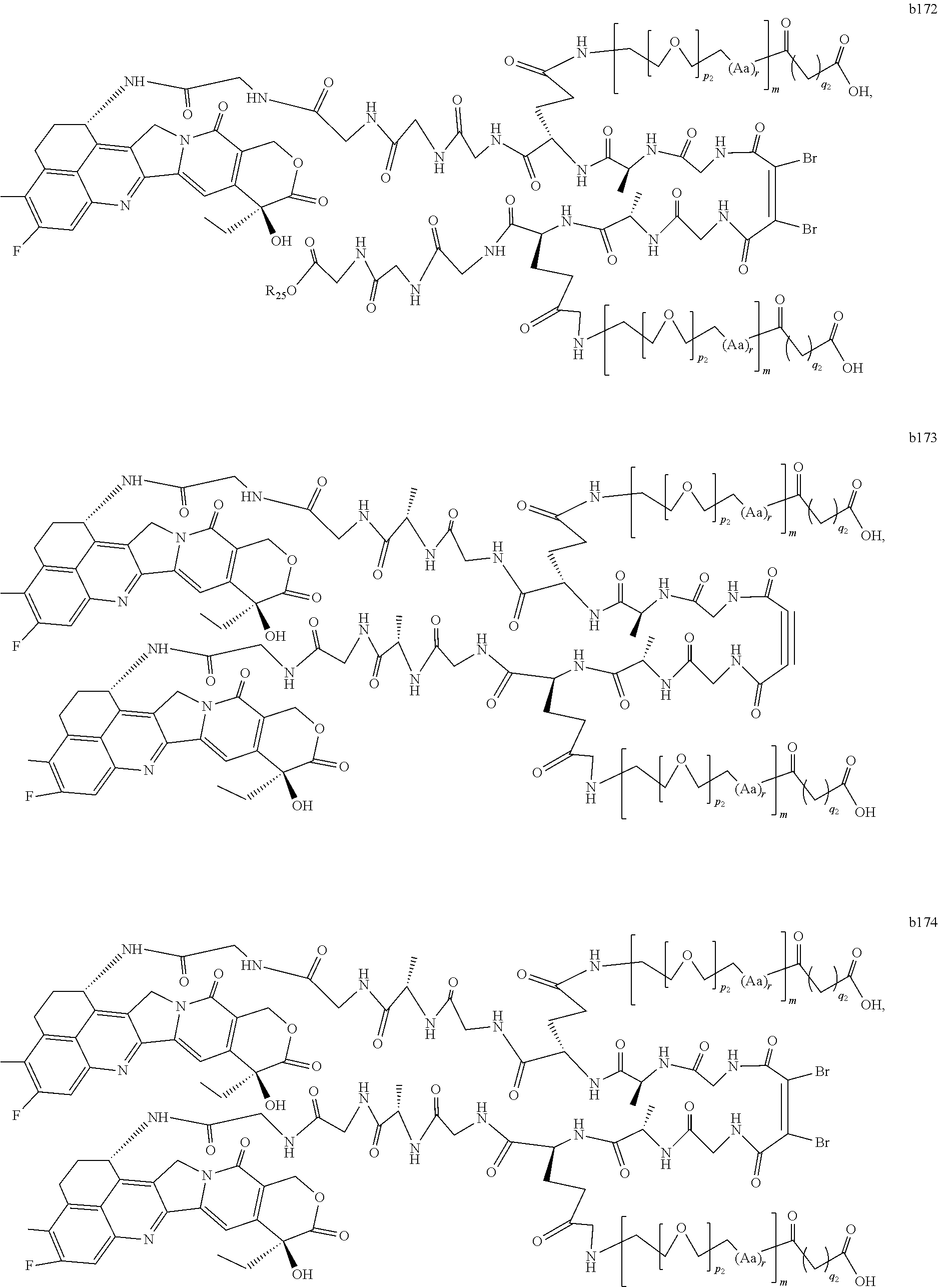 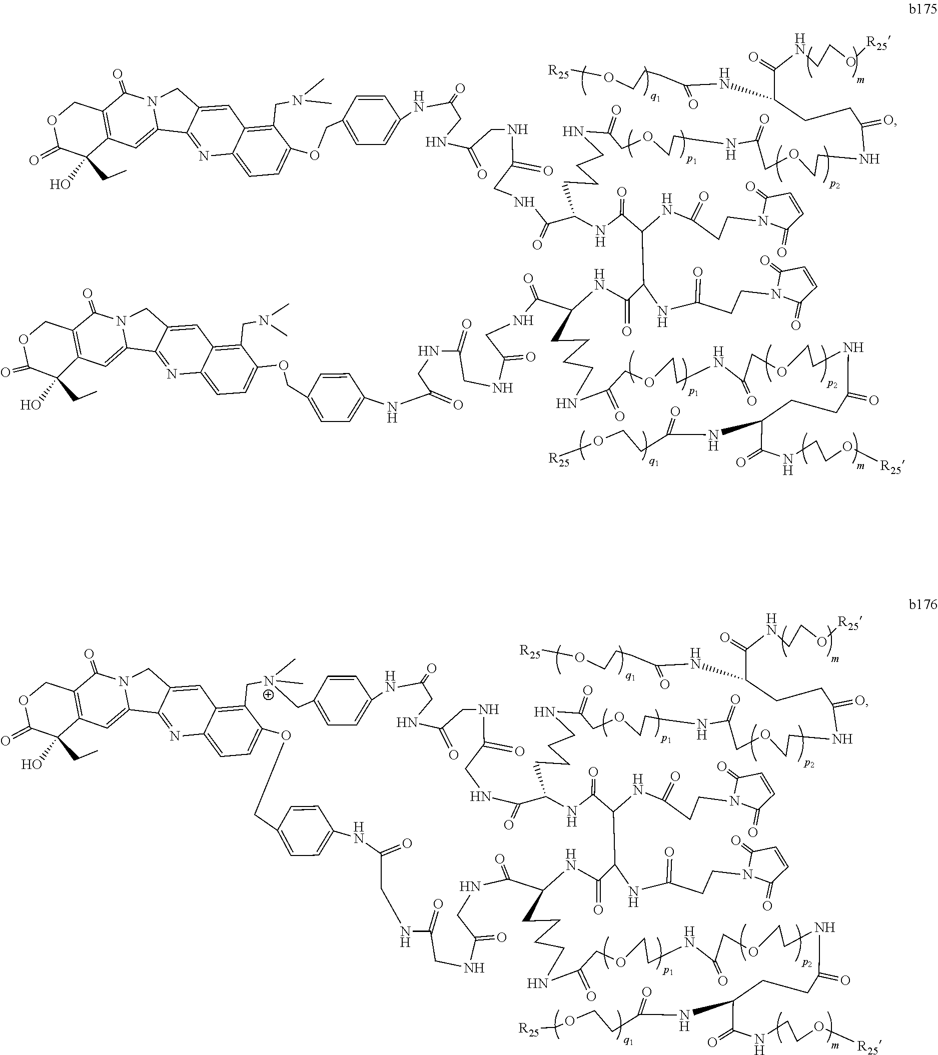 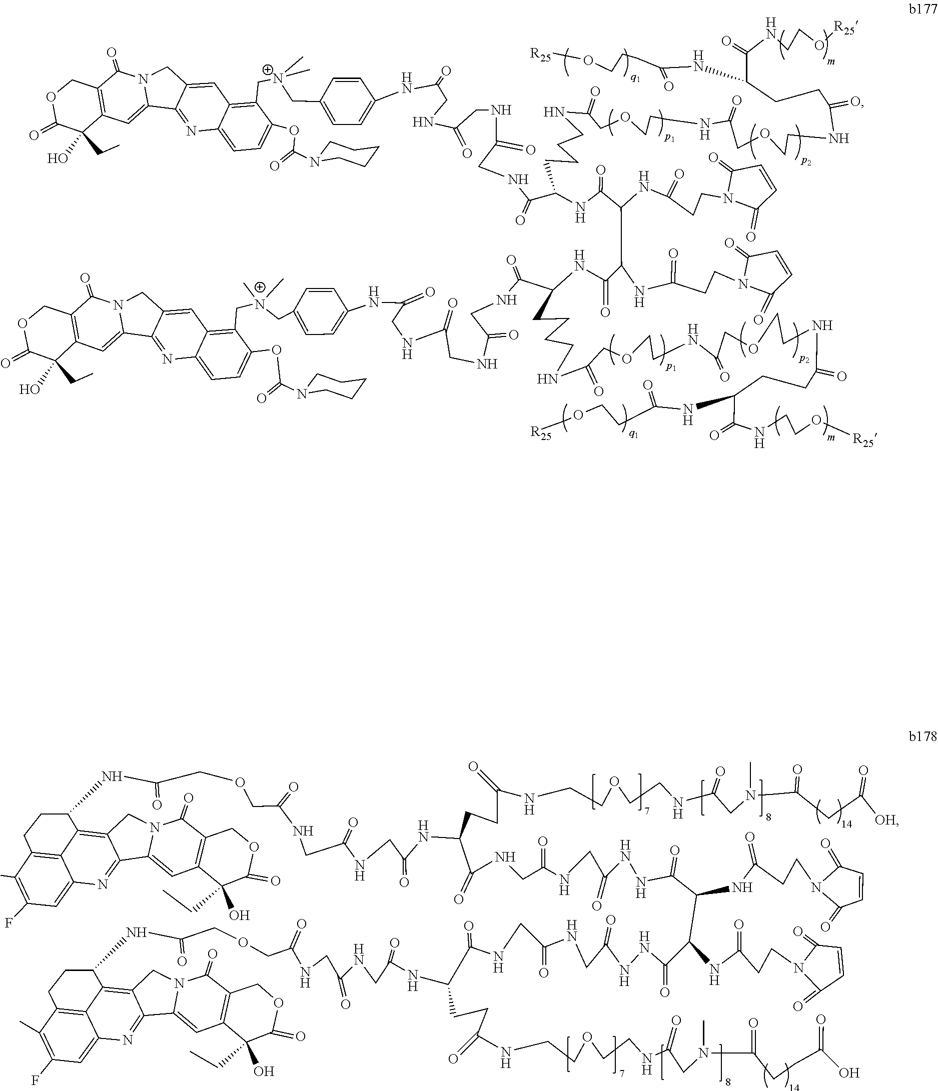 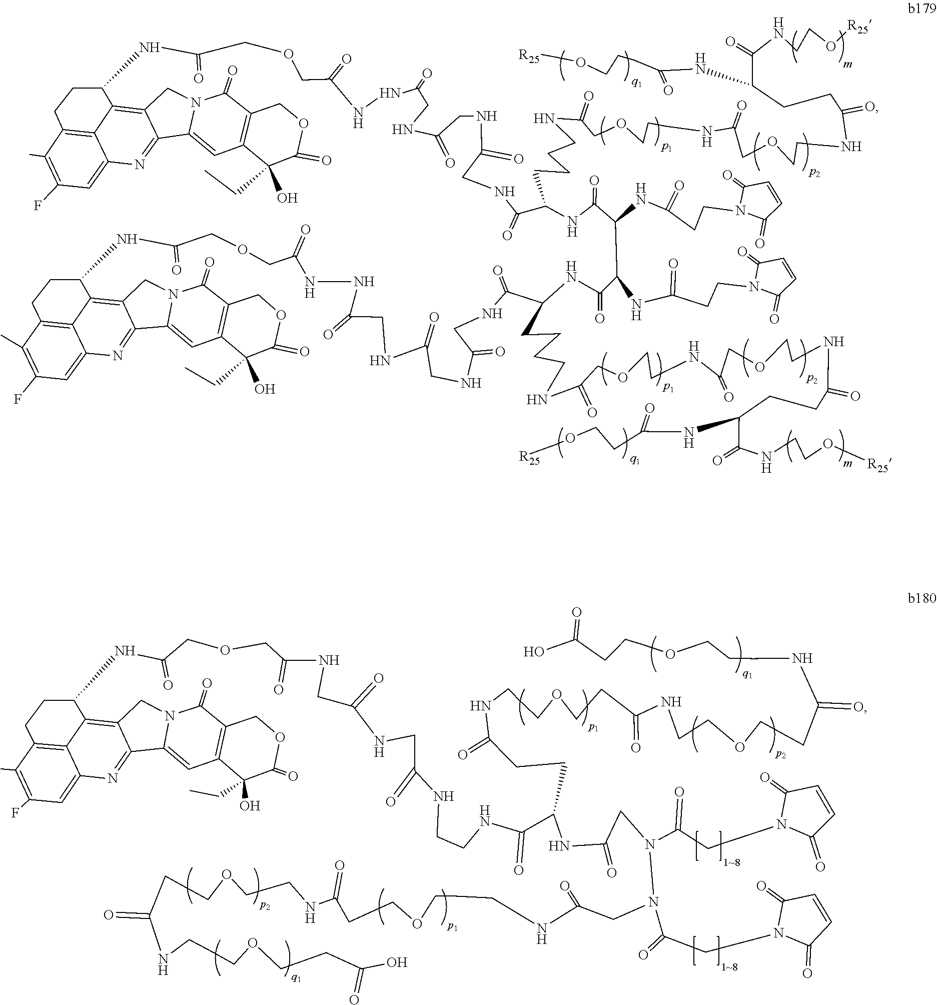 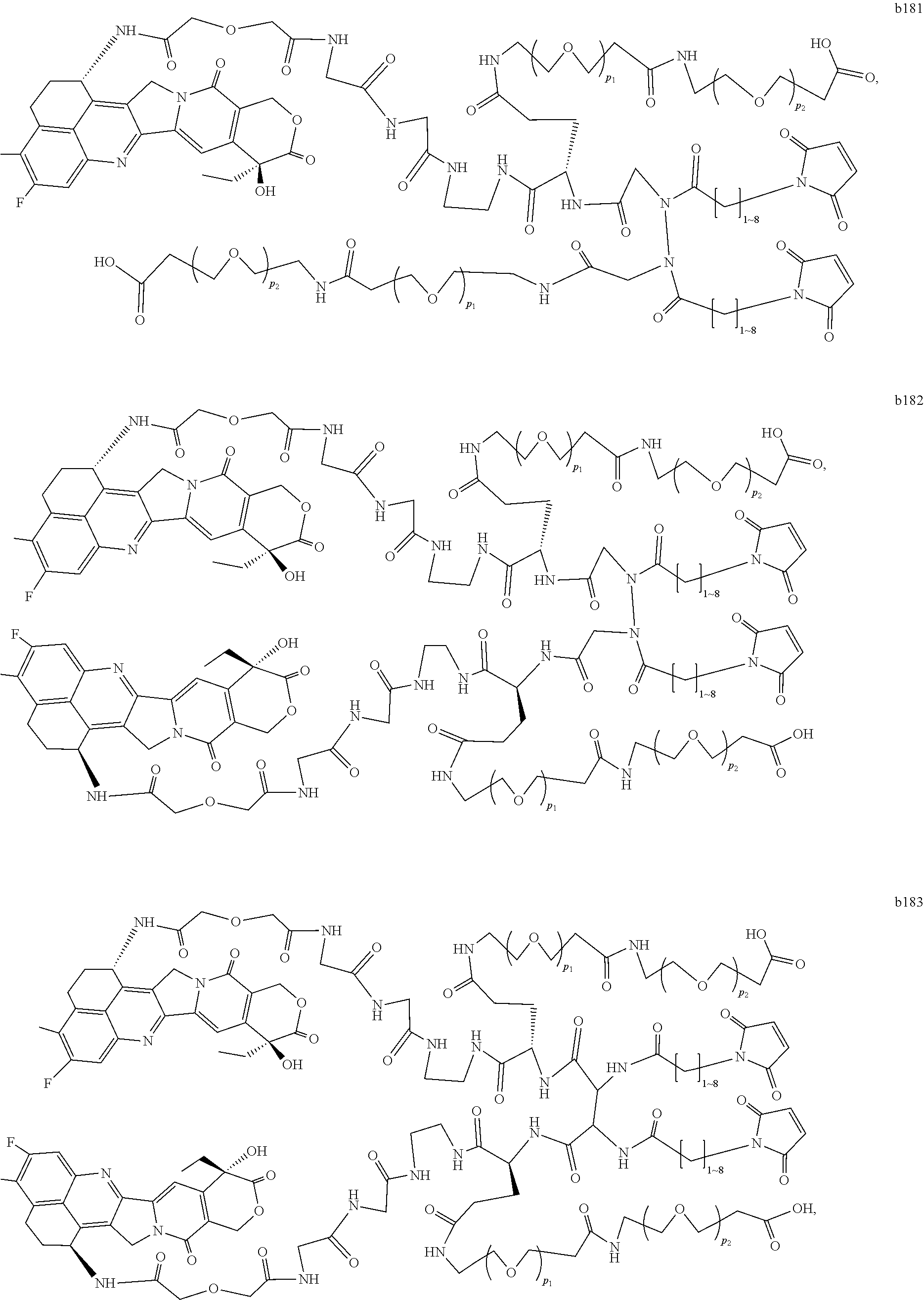 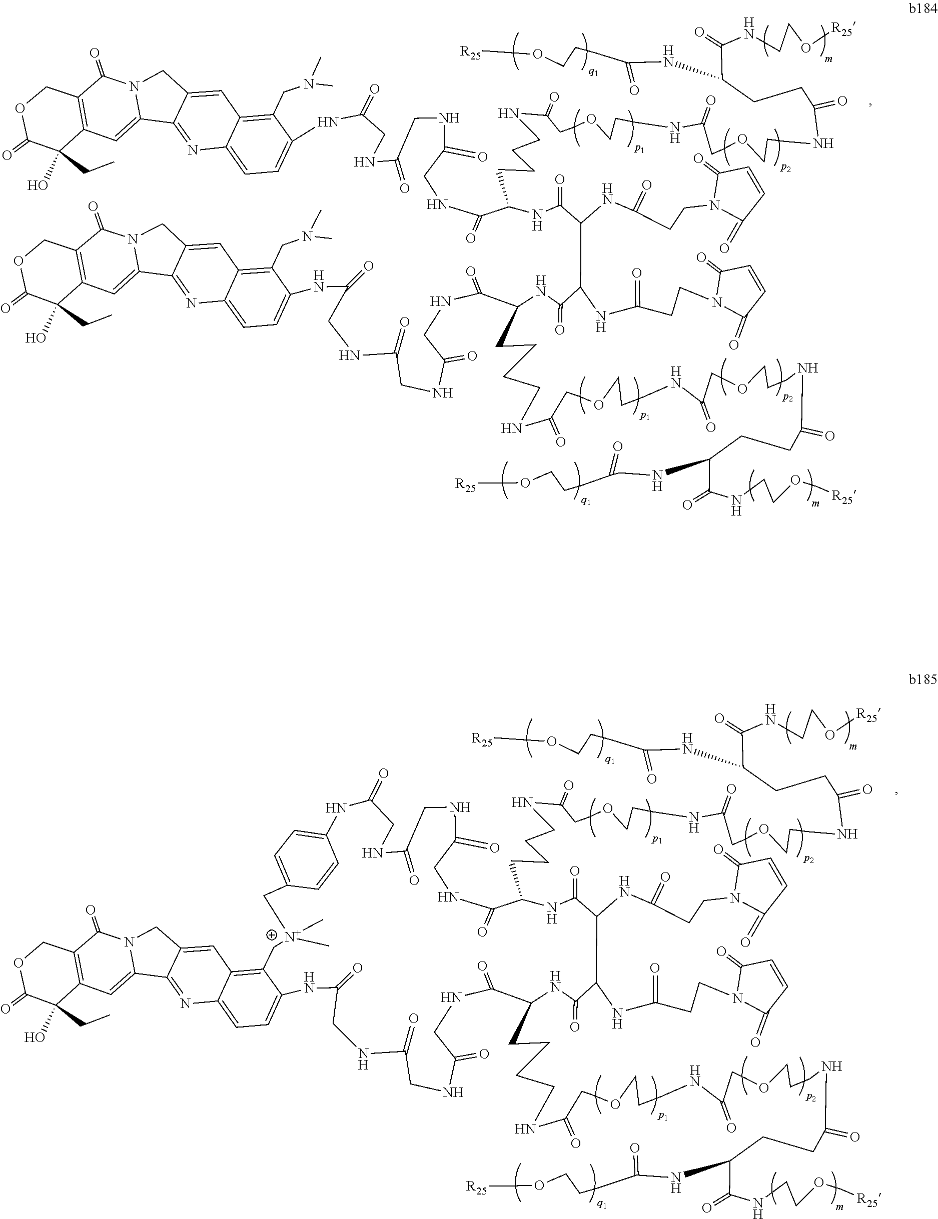 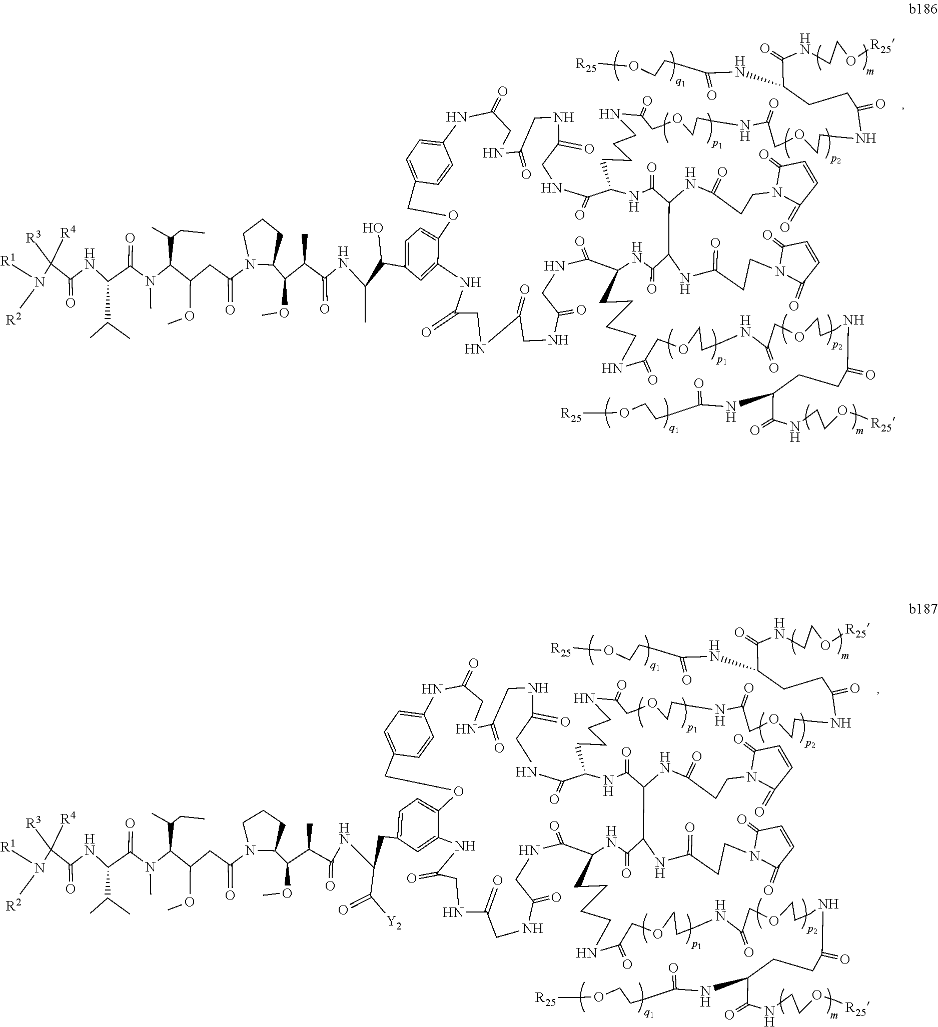 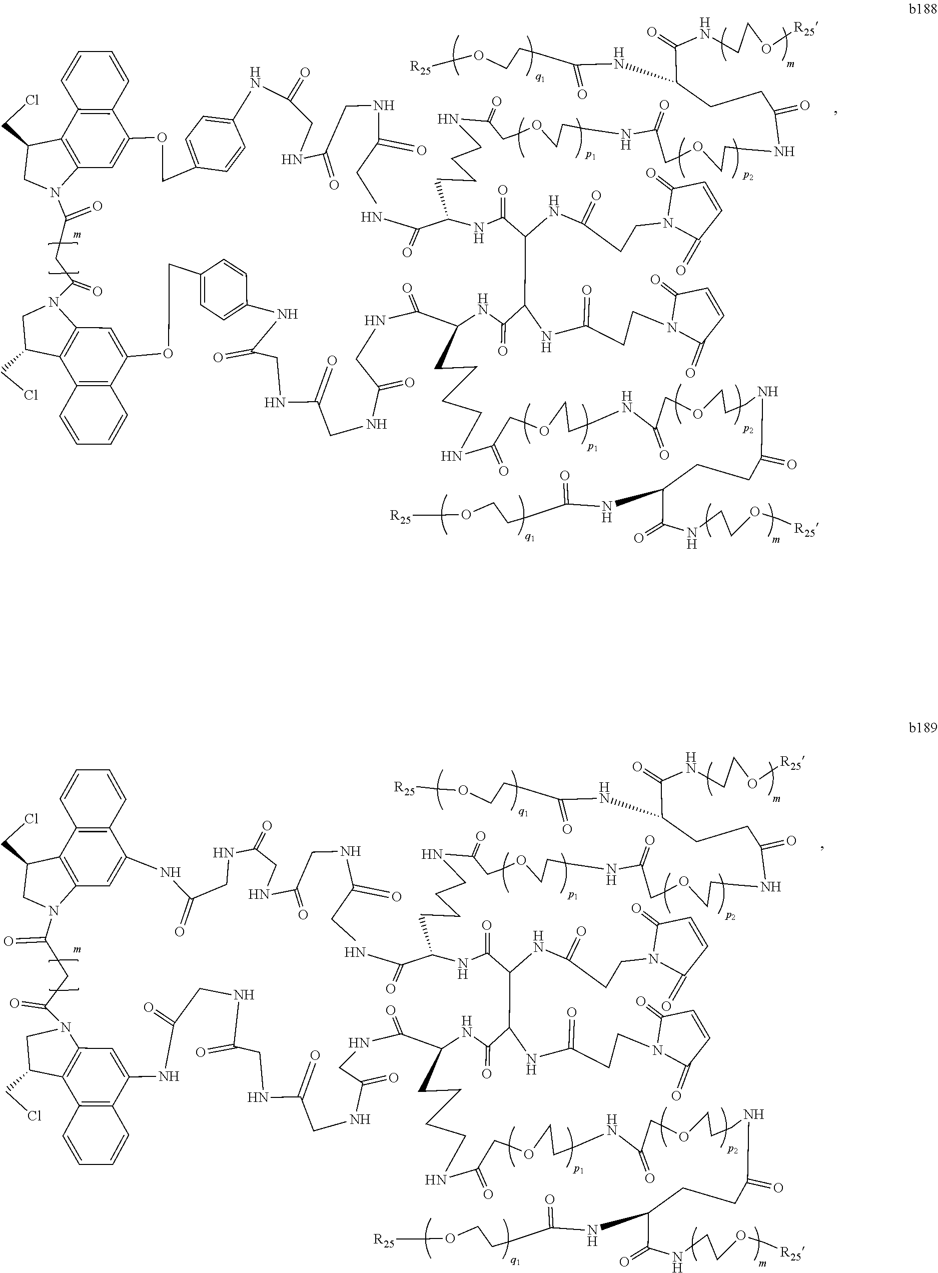 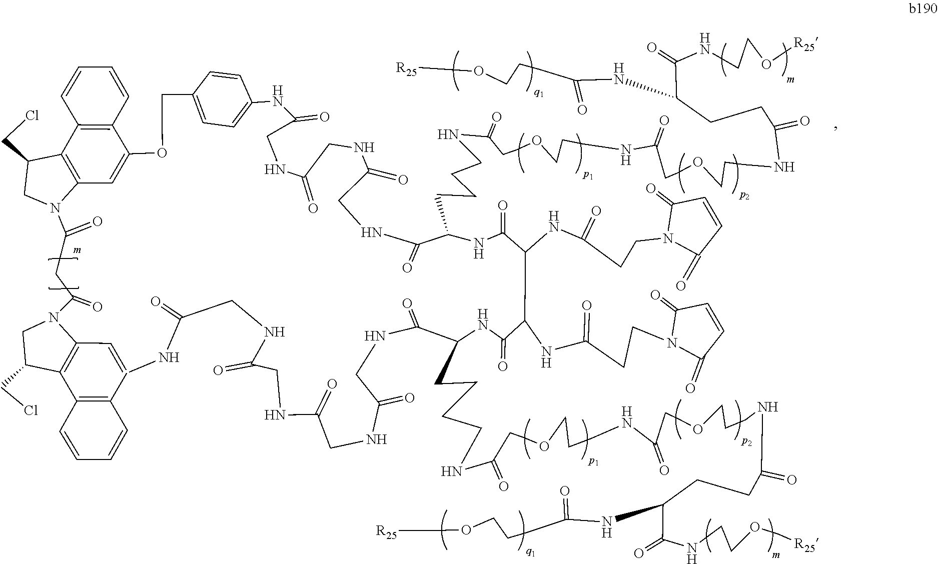 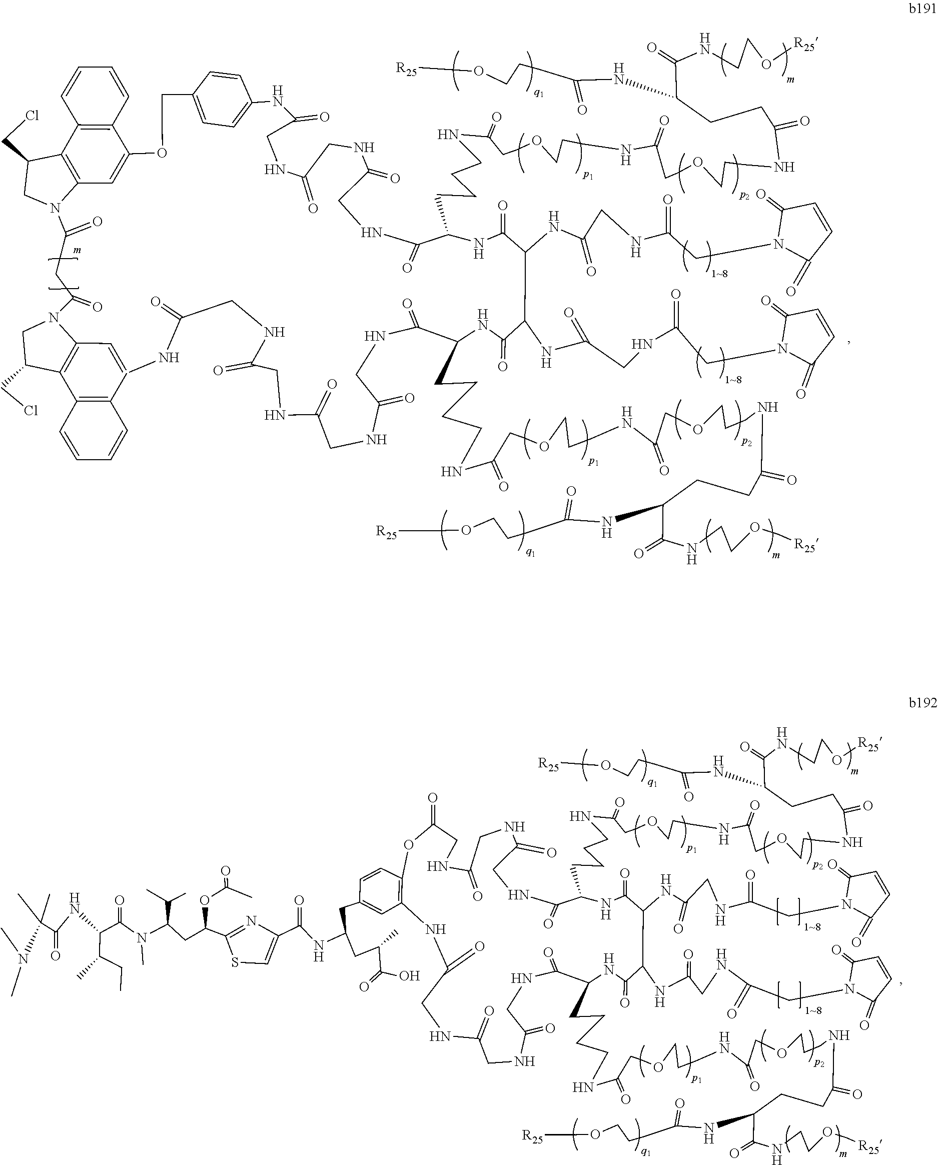 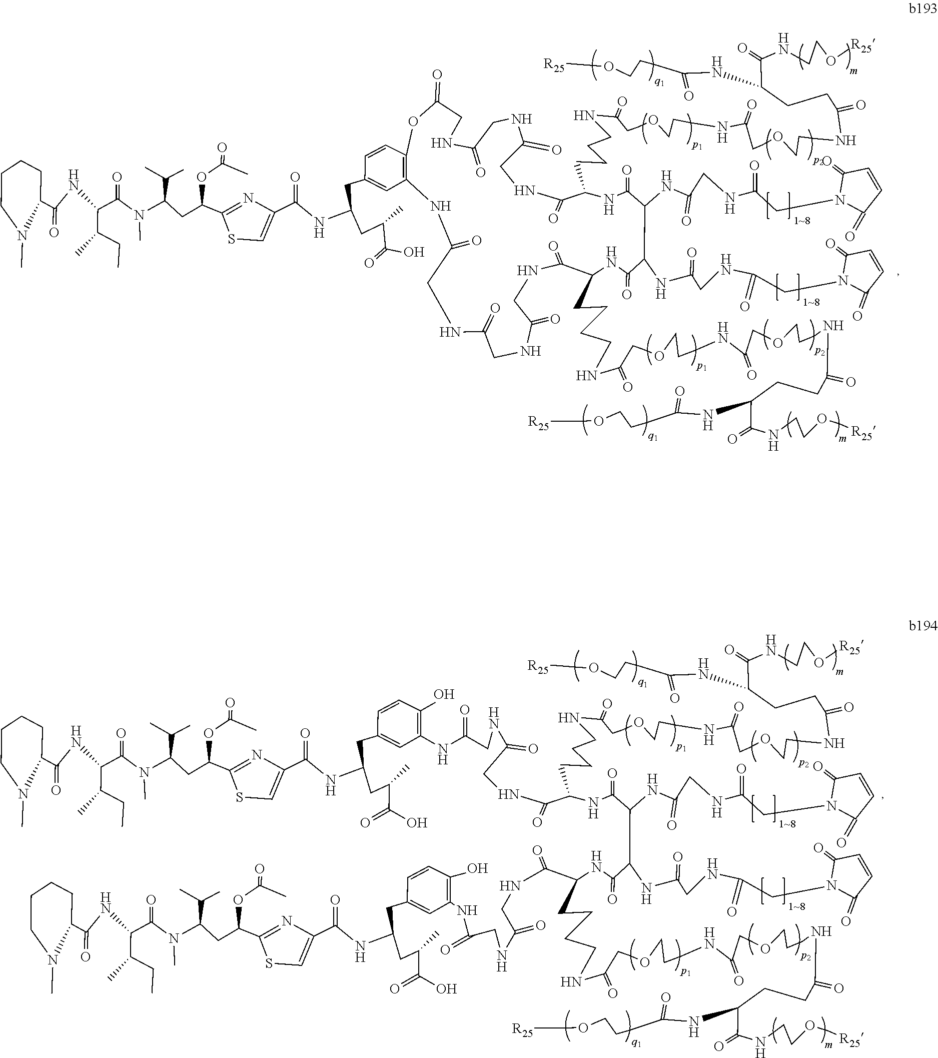 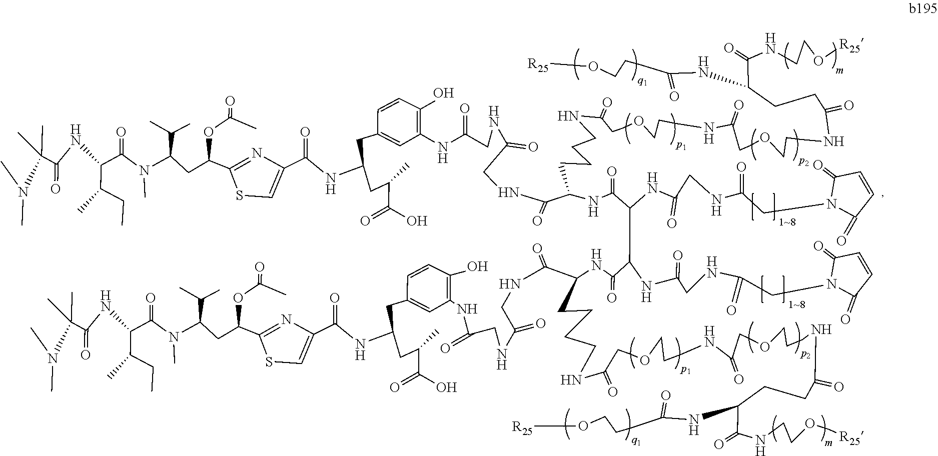 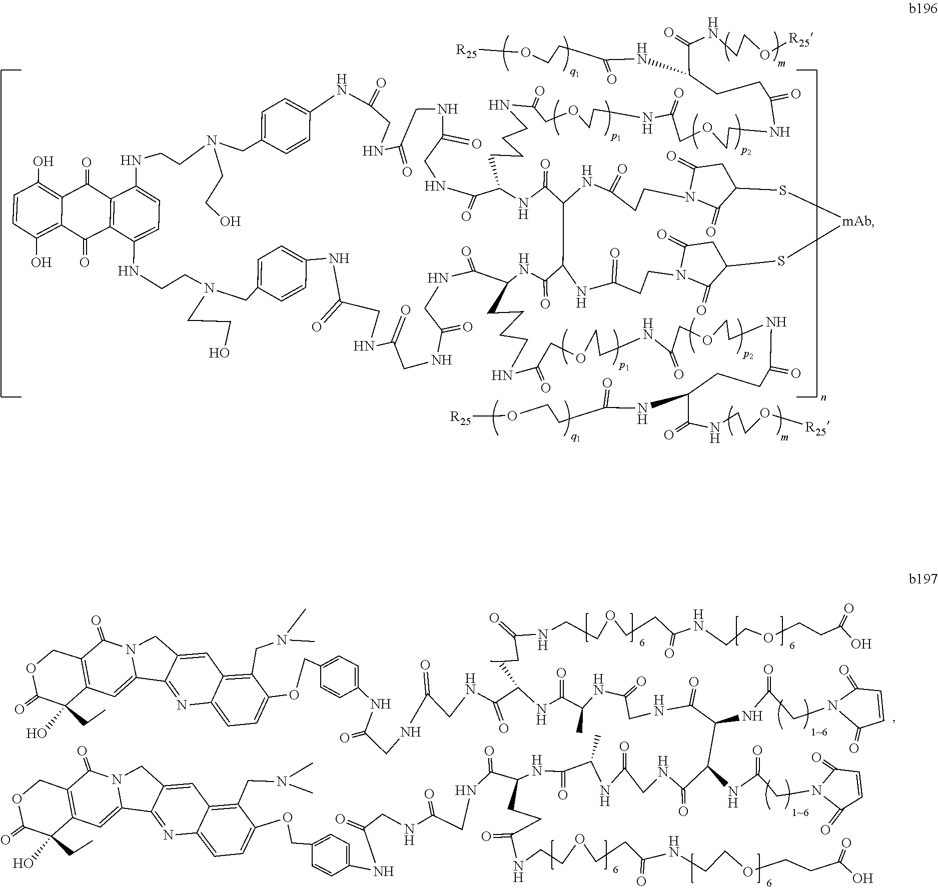 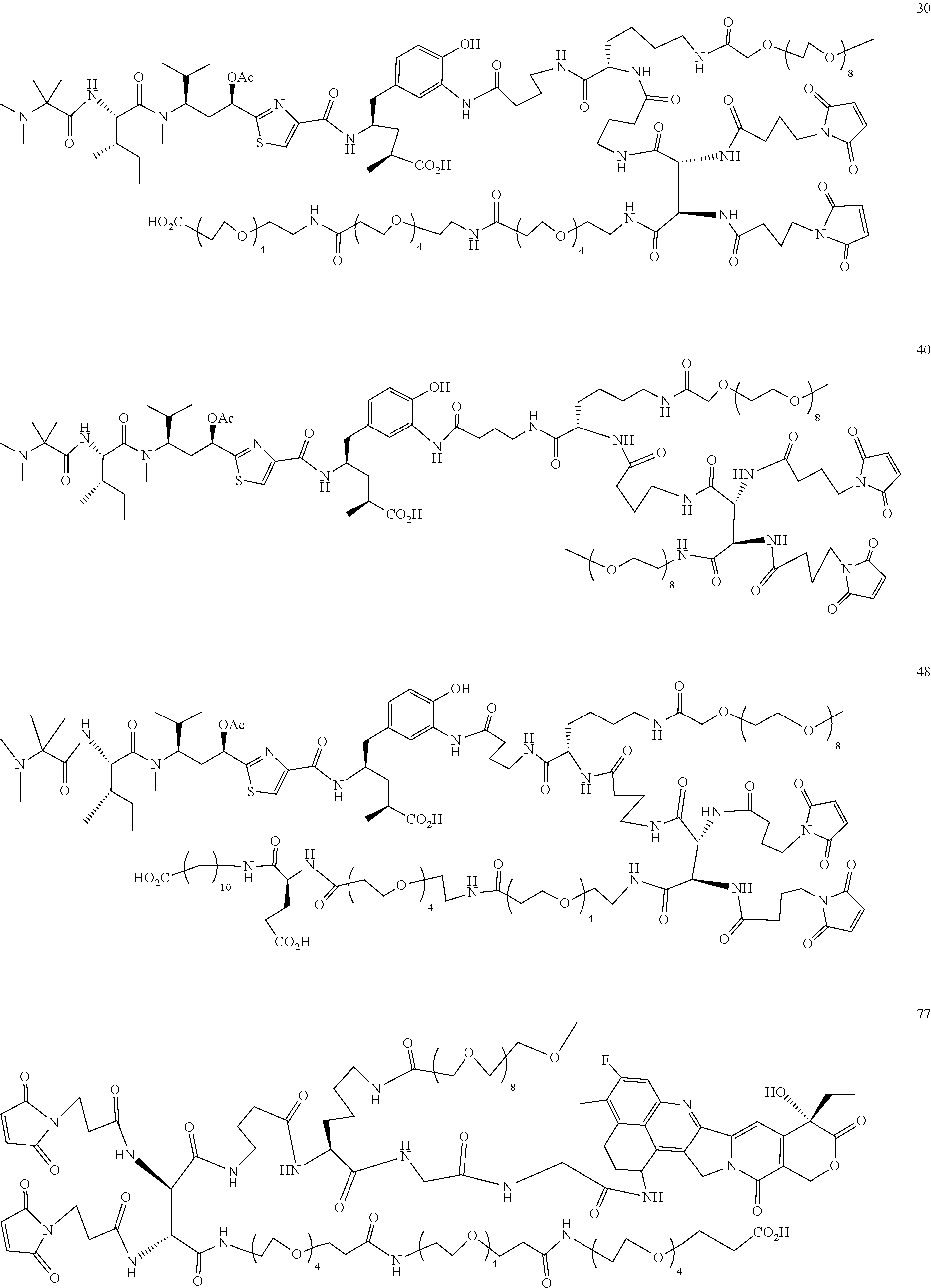 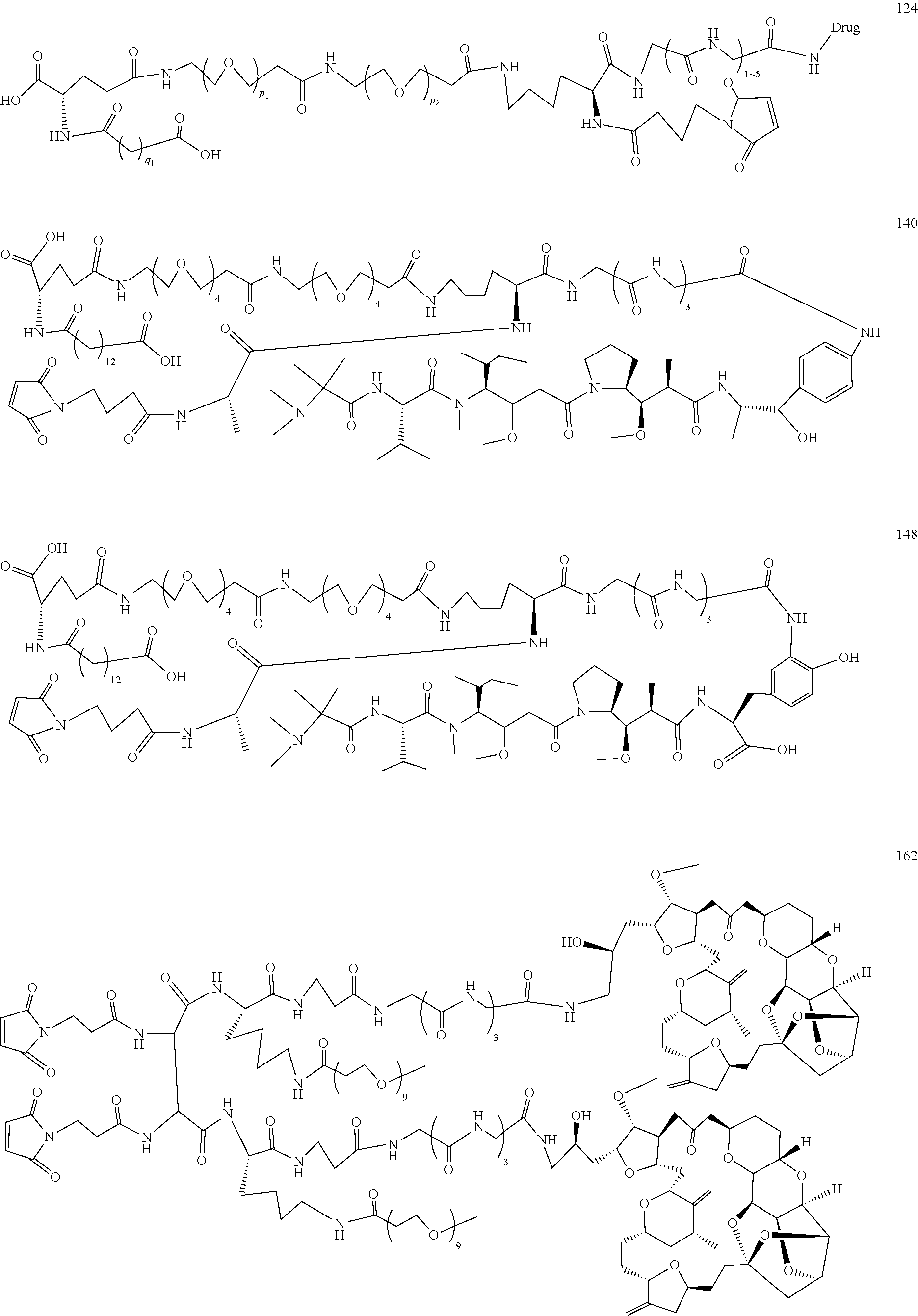 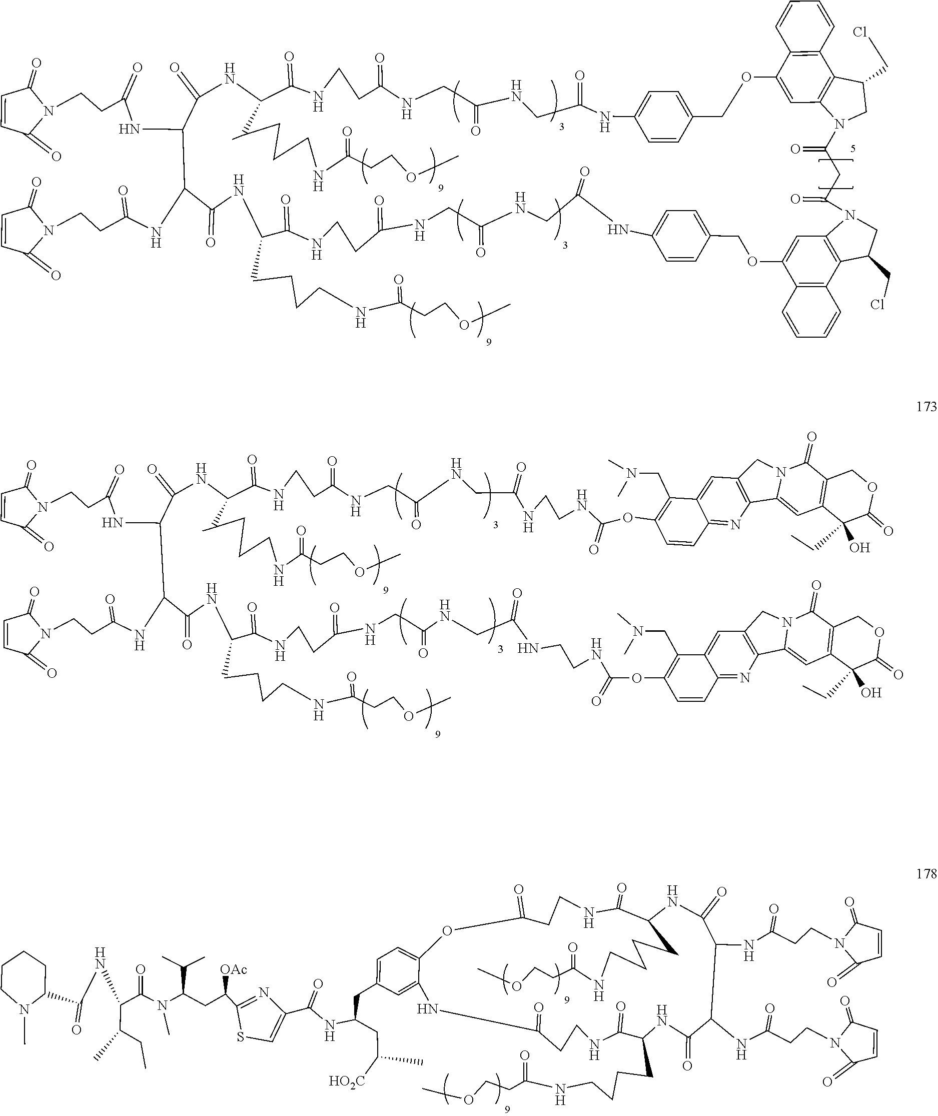 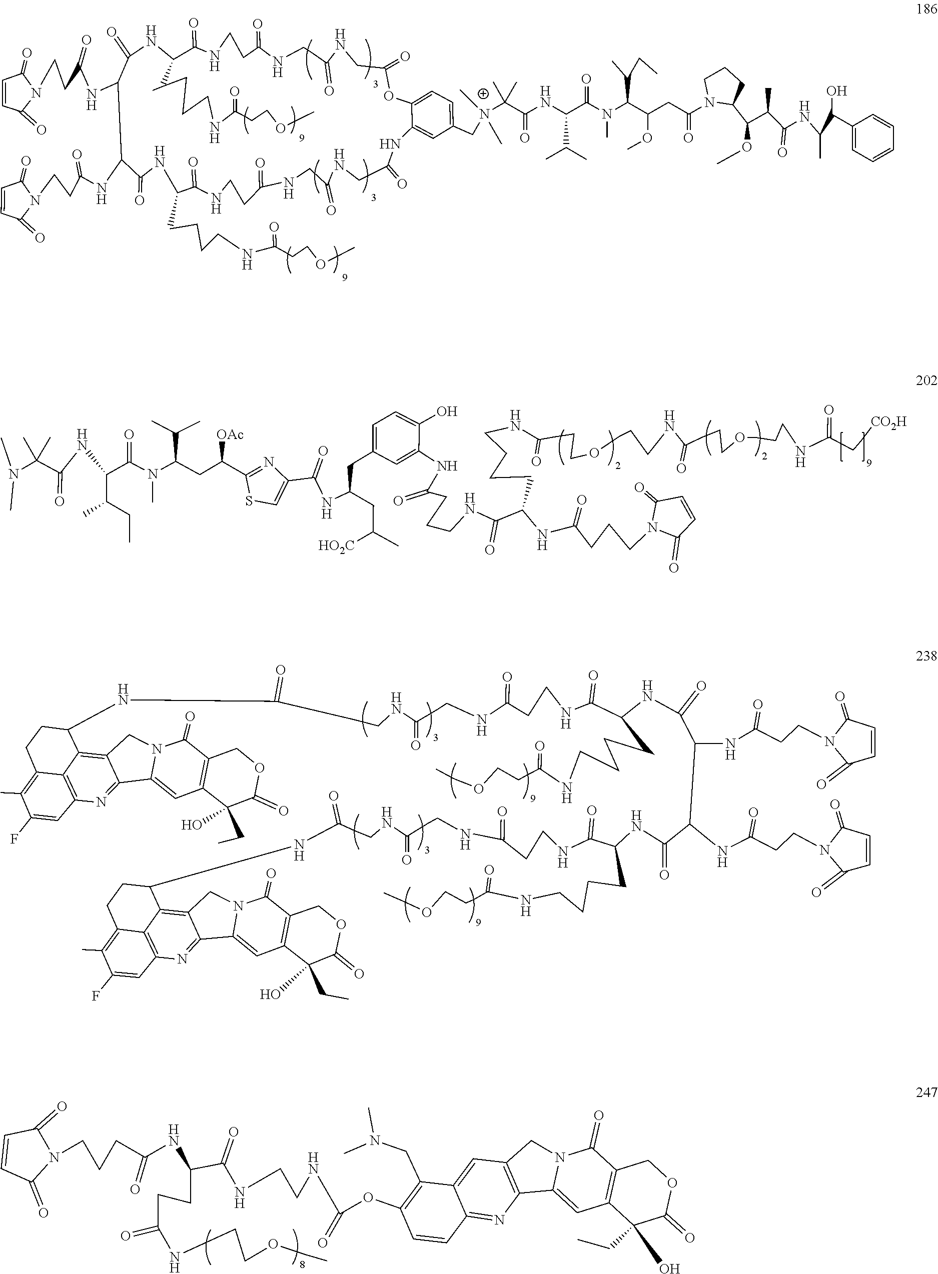 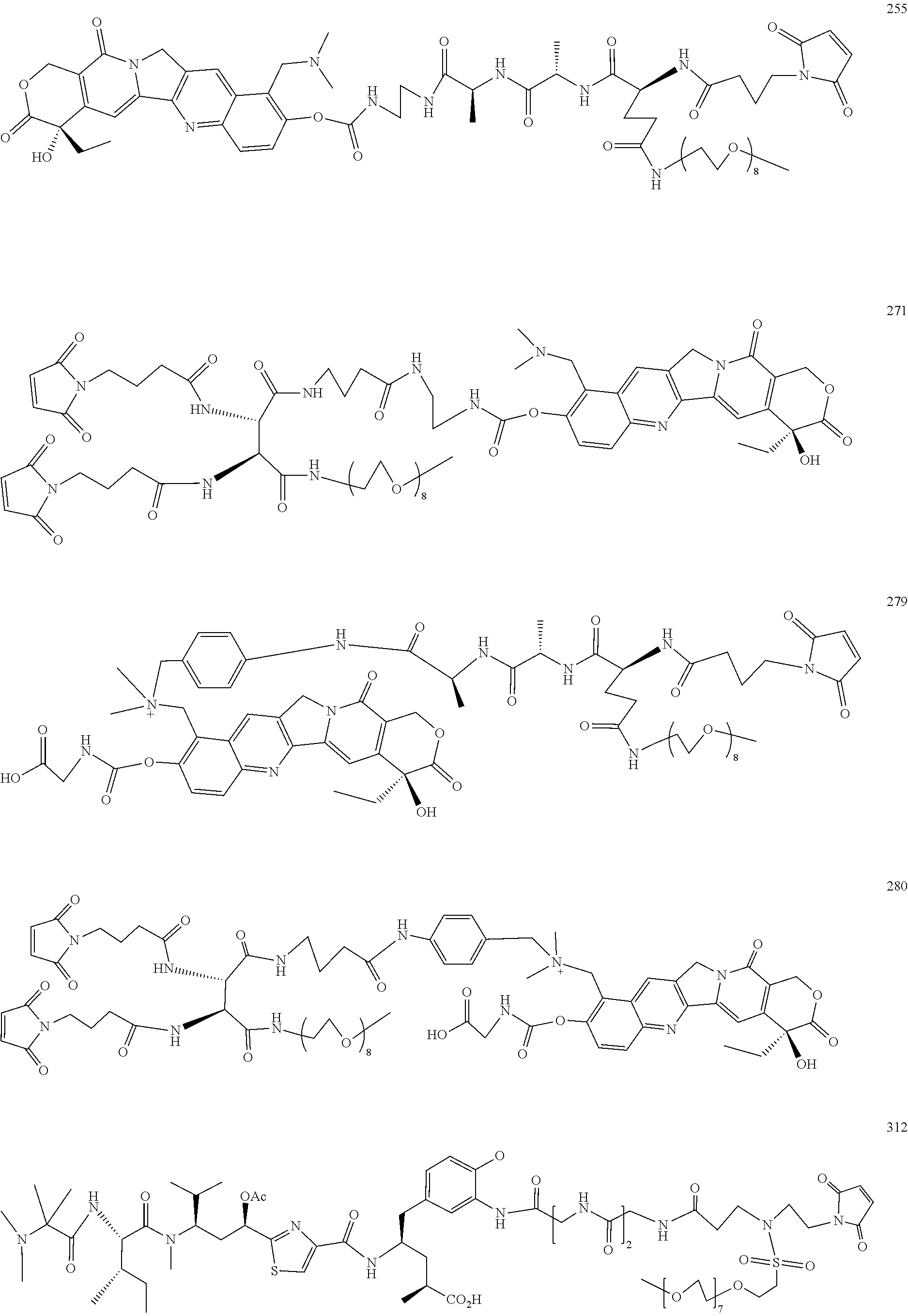 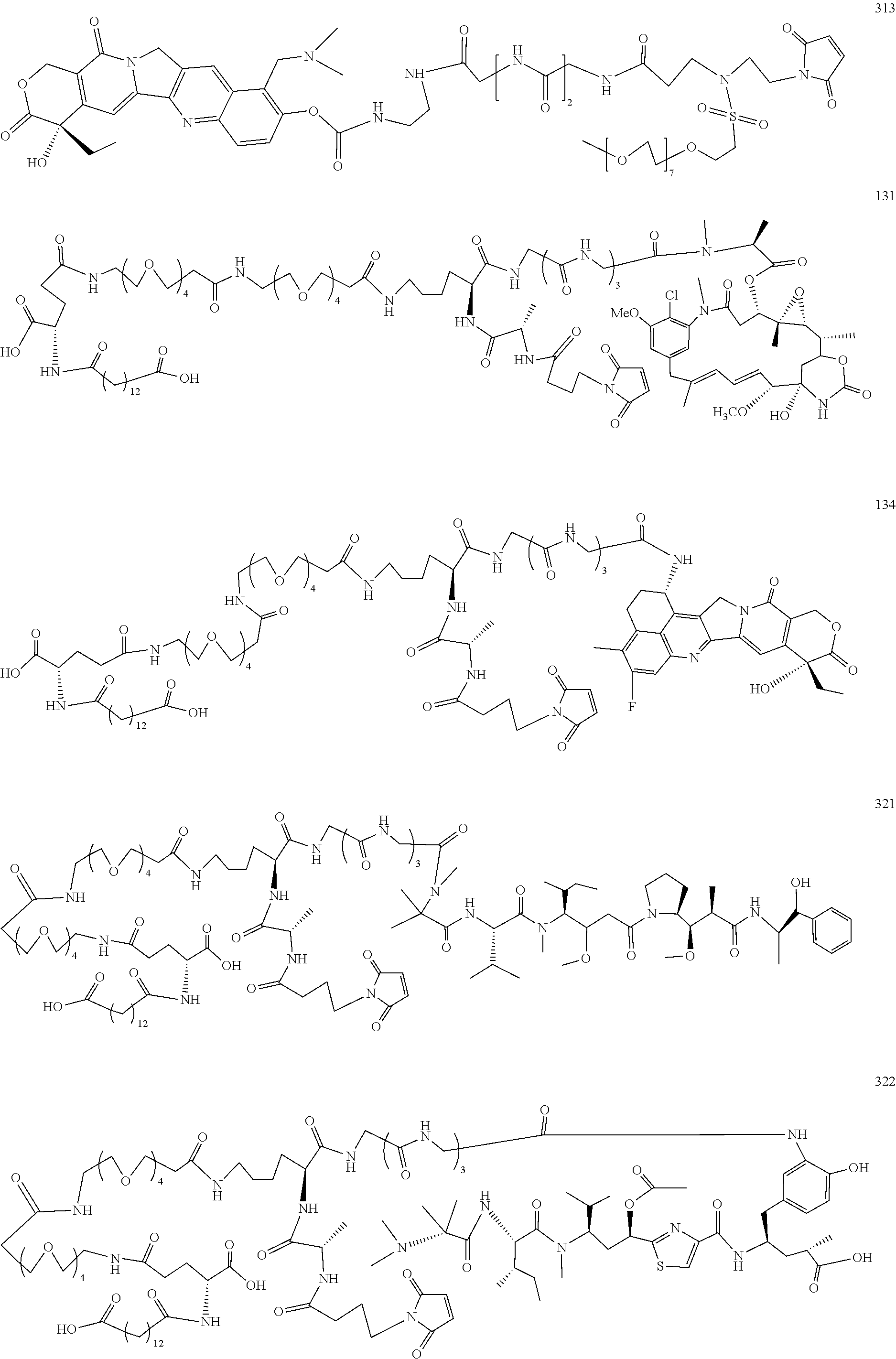
or a derivative thereof with one or more isotopes, or a pharmaceutically acceptable salt, hydrate, or hydrated salt thereof; or a polymorphic crystalline structure thereof; or an optical isomer, racemate, diastereomer or enantiomer thereof; wherein R¹, R², R³, R⁴, R⁵, R₄, R₅, R₇, R₈, R₉, R₁₀, X₁, X₂, X₃, X₄, X₅, X₆, X₈, Y₁, Y₂, Y₃, Y₅, R₁₂, R_{12′}, R₁₃, R₁₃′, R₂₅, R₂₅′, Z₂, Z₃, p, p₁, p₂, p₃, q₁, q₂, Lv₁, Lv₂, Lv₃, Lv_{3′}, m, m₁, n, and mAb are described the same as in claim 24; Aa is natural or unnatural amino acid; r is 0-12; (Aa)r is a peptide containing the same or different sequence of amino acids when r>2; r=0 means (Aa)r absent.

32. The compound according to claim 22, wherein the cell binding agent is selected from:
(A): the group consisting of an antibody, a protein, probody, nanobody, a vitamin (including folate), peptide, a polymeric micelle, a liposome, a lipoprotein-based drug carrier, a nano-particle drug carrier, a dendrimer, and a molecule or a particle of any of said above coating or linking with a cell-binding ligand, or a combination of two or more of the above;
(B): an antibody-like protein, a full-length antibody (polyclonal antibody, monoclonal antibody, antibody dimer, antibody multimer), multispecific antibody (selected from, bispecific antibody, trispecific antibody, or tetraspecific antibody); a single chain antibody, an antibody fragment that binds to the target cell, a monoclonal antibody, a single chain monoclonal antibody, a monoclonal antibody fragment that binds the target cell, a chimeric antibody, a chimeric antibody fragment that binds to the target cell, a domain antibody, a domain antibody fragment that binds to the target cell, a resurfaced antibody, a resurfaced single chain antibody, or a resurfaced antibody fragment that binds to the target cell, a humanized antibody or a resurfaced antibody, a humanized single chain antibody, or a humanized antibody fragment that binds to the target cell, anti-idiotypic (anti-Id) antibodies, CDR's, diabody, triabody, tetrabody, miniantibody, a probody, a probody fragment, small immune proteins (SIP), a lymphokine, a hormone, a vitamin, a growth factor, a colony stimulating factor, a nutrient-transport molecule, large molecular weight protein, fusion protein, kinase inhibitor, gene-targeting agent, nanoparticle or polymer modified with antibody or large molecular weight protein;
(C): a cell-binding ligand or receptor agonist selected from: Folate derivatives; Glutamic acid urea derivatives; Somatostatin and its analogs (selected from the group consisting of octreotide (Sandostatin) and lanreotide (Somatuline)); Aromatic sulfonamides; Pituitary adenylate cyclase activating peptides (PACAP) (PAC1); Vasoactive intestinal peptides (VIP/PACAP) (VPAC1, VPAC2); Melanocyte-stimulating hormones (α-MSH); Cholecystokinins (CCK)/gastrin receptor agonists; Bombesins (selected from the group consisting of Pyr-Gln-Arg-Leu-Gly-Asn-Gln-Trp-Ala-Val-Gly-His-Leu-Met-NH₂)/gastrin-releasing peptide (GRP); Neurotensin receptor ligands (NTR1, NTR2, NTR3); Substance P (NK1 receptor) ligands; Neuropeptide Y (Y1-Y6); Homing Peptides include RGD (Arg-Gly-Asp), NGR (Asn-Gly-Arg), dimeric and multimeric cyclic RGD peptides (selected from cRGDfV), TAASGVRSMH and LTLRWVGLMS (Chondroitin sulfate proteoglycan NG2 receptor ligands) and F3 peptides; Cell Penetrating Peptides (CPPs); Peptide Hormones selected from the group consisting of luteinizing hormone-releasing hormone (LHRH) agonists and antagonists, and gonadotropin-releasing hormone (GnRH) agonist, selected from the group consisting of buserelin (Pyr-His-Trp-Ser-Tyr-D-Ser(OtBu)-Leu-Arg-Pro-NHEt), Gonadorelin (Pyr-His-Trp-Ser-Tyr-Gly-Leu-Arg-Pro-Gly-NH₂), Goserelin (Pyr-His-Trp-Ser-Tyr-D-Ser(OtBu)-Leu-Arg-Pro-AzGly-NH₂), Histrelin (Pyr-His-Trp-Ser-Tyr-D-His(N-benzyl)-Leu-Arg-Pro-NHEt), leuprolide (Pyr-His-Trp-Ser-Tyr-D-Leu-Leu-Arg-Pro-NHEt), Nafarelin (Pyr-His-Trp-Ser-Tyr-2Nal-Leu-Arg-Pro-Gly-NH₂), Triptorelin (Pyr-His-Trp-Ser-Tyr-D-Trp-Leu-Arg-Pro-Gly-NH₂), Nafarelin, Deslorelin, Abarelix (Ac-D-2Nal-D-4-chloroPhe-D-3-(3-pyridyl)Ala-Ser-(N-Me)Tyr-D-Asn-Leu-isopropylLys-Pro-DAla-NH₂), Cetrorelix (Ac-D-2Nal-D-4-chloroPhe-D-3-(3-pyridyl)Ala-Ser-Tyr-D-Cit-Leu-Arg-Pro-D-Ala-NH₂), Degarelix (Ac-D-2Nal-D-4-chloroPhe-D-3-(3-pyridyl)Ala-Ser-4-aminoPhe(L-hydroorotyl)-D-4-aminoPhe(carba-moyl)-Leu-isopropylLys-Pro-D-Ala-NH₂), and Ganirelix (Ac-D-2Nal-D-4-chloroPhe-D-3-(3-pyridyl)Ala-Ser-Tyr-D-(N9, N10-diethyl)-homoArg-Leu-(N9, N10-diethyl)-homoArg-Pro-D-Ala-NH₂); Pattern Recognition Receptor (PRRs), selected from the group consisting of Toll-like receptors' (TLRs) ligands, C-type lectins and Nodlike Receptors' (NLRs) ligands; Calcitonin receptor agonists; integrin receptors' and their receptor subtypes' (selected from the group consisting of α_{V}β₁, α_{V}β₃, α_{V}β₅, α_{V}β₆, α₆β₄, α₇β₁, α_{L}β₂, α_{IIb}β₃) agonists (selected from the group consisting of GRGDSPK, cyclo(RGDfV) (L1) and its derives [cyclo(—N(Me)R-GDfV), cyclo(R-Sar-DfV), cyclo(RG-N(Me)D-fV), cyclo(RGD-N(Me)f-V), cyclo(RGDf-N(Me)V-)(Cilengitide)]; Nanobody (a derivative of VHH (camelid Ig)); Domain antibodies (dAb, a derivative of VH or VL domain); Bispecific T cell Engager (BiTE, a bispecific diabody); Dual Affinity ReTargeting (DART, a bispecific diabody); Tetravalent tandem antibodies (TandAb, a dimerized bispecific diabody); Anticalin (a derivative of Lipocalins); Adnectins (10th FN3 (Fibronectin)); Designed Ankyrin Repeat Proteins (DARPins); Avimers; EGF receptors, or VEGF receptors' agonists;
(D): a small molecule of cell-binding molecule/ligand or a cell receptor agonist selected from the group consisting of LB01 (Folate), LB02 (PMSA ligand), LB03 (PMSA ligand), LB04 (PMSA ligand), LB05 (Somatostatin), LB06 (Somatostatin), LB07 (Octreotide, a Somatostatin analog), LB08 (Lanreotide, a Somatostatin analog), LB09 (Vapreotide (Sanvar), a Somatostatin analog), LB10 (CAIX ligand), LB11 (CAIX ligand), LB12 (Gastrin releasing peptide receptor (GRPr), MBA), LB13 (luteinizing hormone-releasing hormone (LH-RH) ligand and GnRH), LB14 (luteinizing hormone-releasing hormone (LH-RH) and GnRH ligand), LB15 (GnRH antagonist, Abarelix), LB16 (cobalamin, vitamin B12 analog), LB17 (cobalamin, vitamin B12 analog), LB18 (for αᵥβ₃ integrin receptor, cyclic RGD pentapeptide), LB19 (hetero-bivalent peptide ligand for VEGF receptor), LB20 (Neuromedin B), LB21 (bombesin for a G-protein coupled receptor), LB22 (TLR₂ for a Toll-like receptor,), LB23 (for an androgen receptor), LB24 (Cilengitide/cyclo(-RGDfV-) for an αᵥ integrin receptor, LB23 (Fludrocortisone), LB25 (Rifabutin analog), LB26 (Rifabutin analog), LB27 (Rifabutin analog), LB28 (Fludrocortisone), LB29 (Dexamethasone), LB30 (fluticasone propionate), LB31 (Beclometasone dipropionate), LB32 (Triamcinolone acetonide), LB33 (Prednisone), LB34 (Prednisolone), LB35 (Methylprednisolone), LB36 (Betamethasone), LB37 (Irinotecan analog), LB38 (Crizotinib analog), LB39 (Bortezomib analog), LB40 (Carfilzomib analog), LB41 (Carfilzomib analog), LB42 (Leuprolide analog), LB43 (Triptorelin analog), LB44 (Clindamycin), LB45 (Liraglutide analog), LB46 (Semaglutide analog), LB47 (Retapamulin analog), LB48 (Indibulin analog), LB49 (Vinblastine analog), LB50 (Lixisenatide analog), LB51 (Osimertinib analog), LB52 (a nucleoside analog), LB53 (Erlotinib analog) and LB54 (Lapatinib analog) which are shown in the following structures: 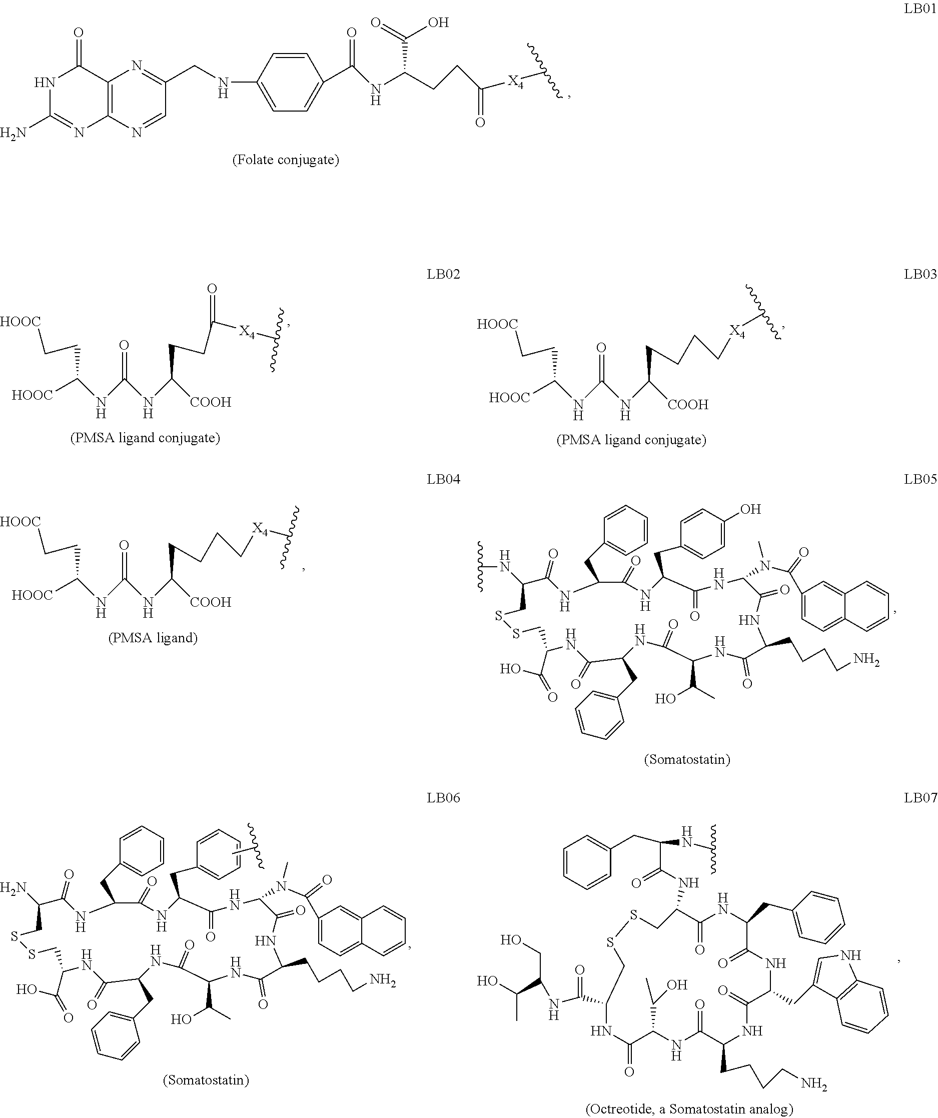 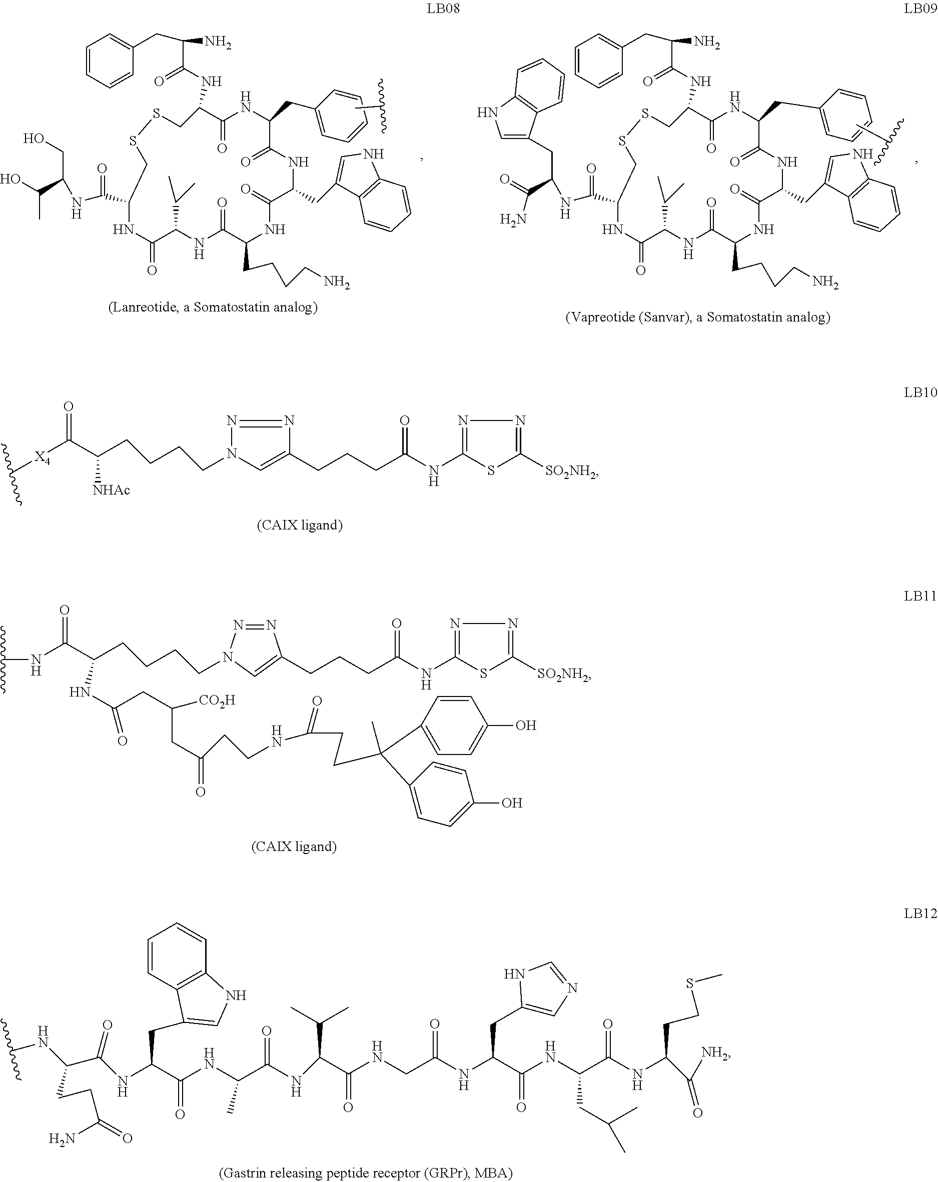 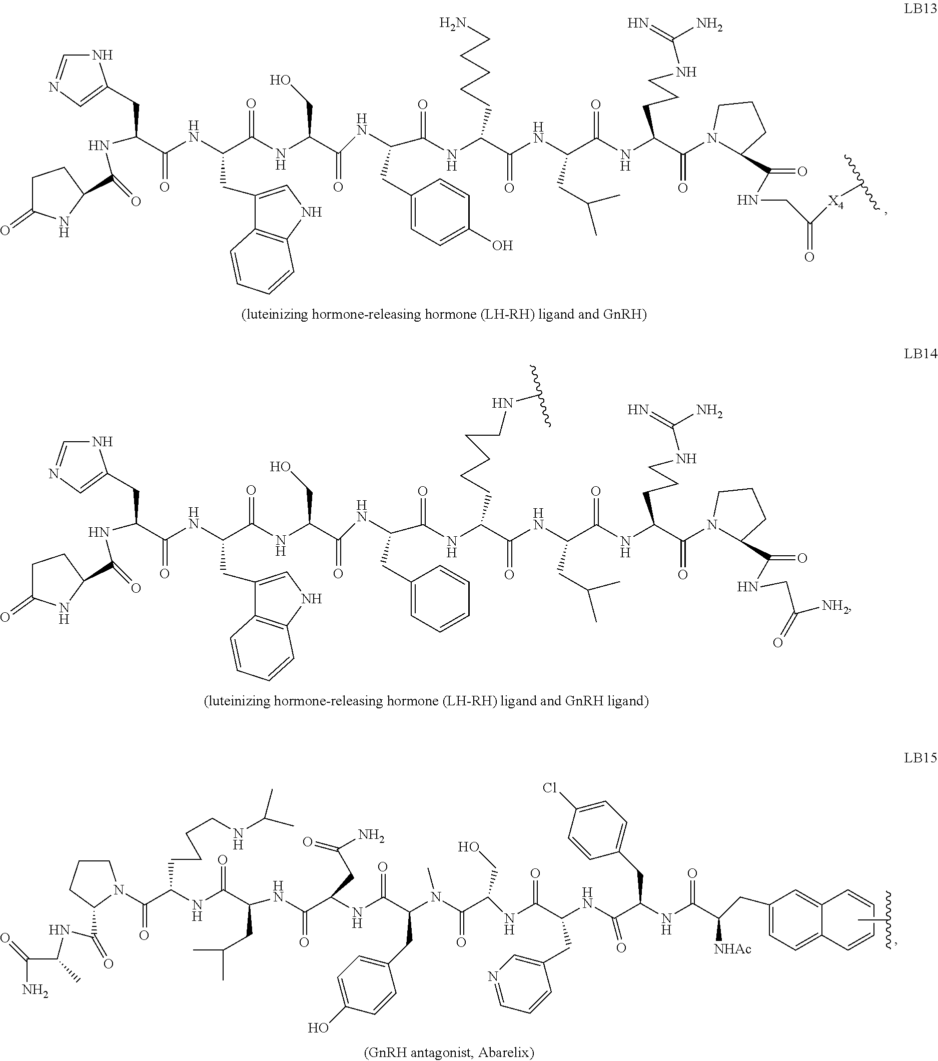 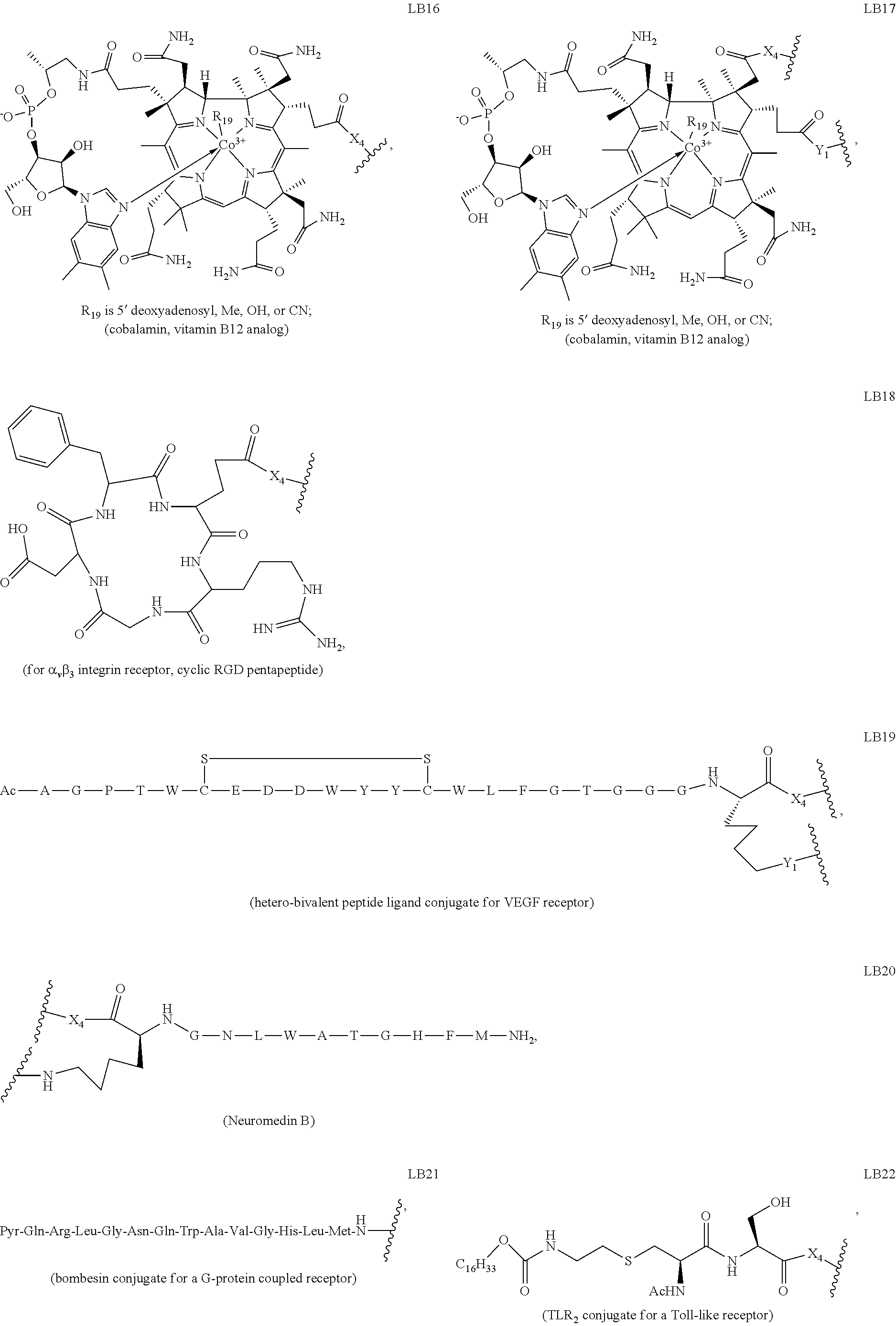 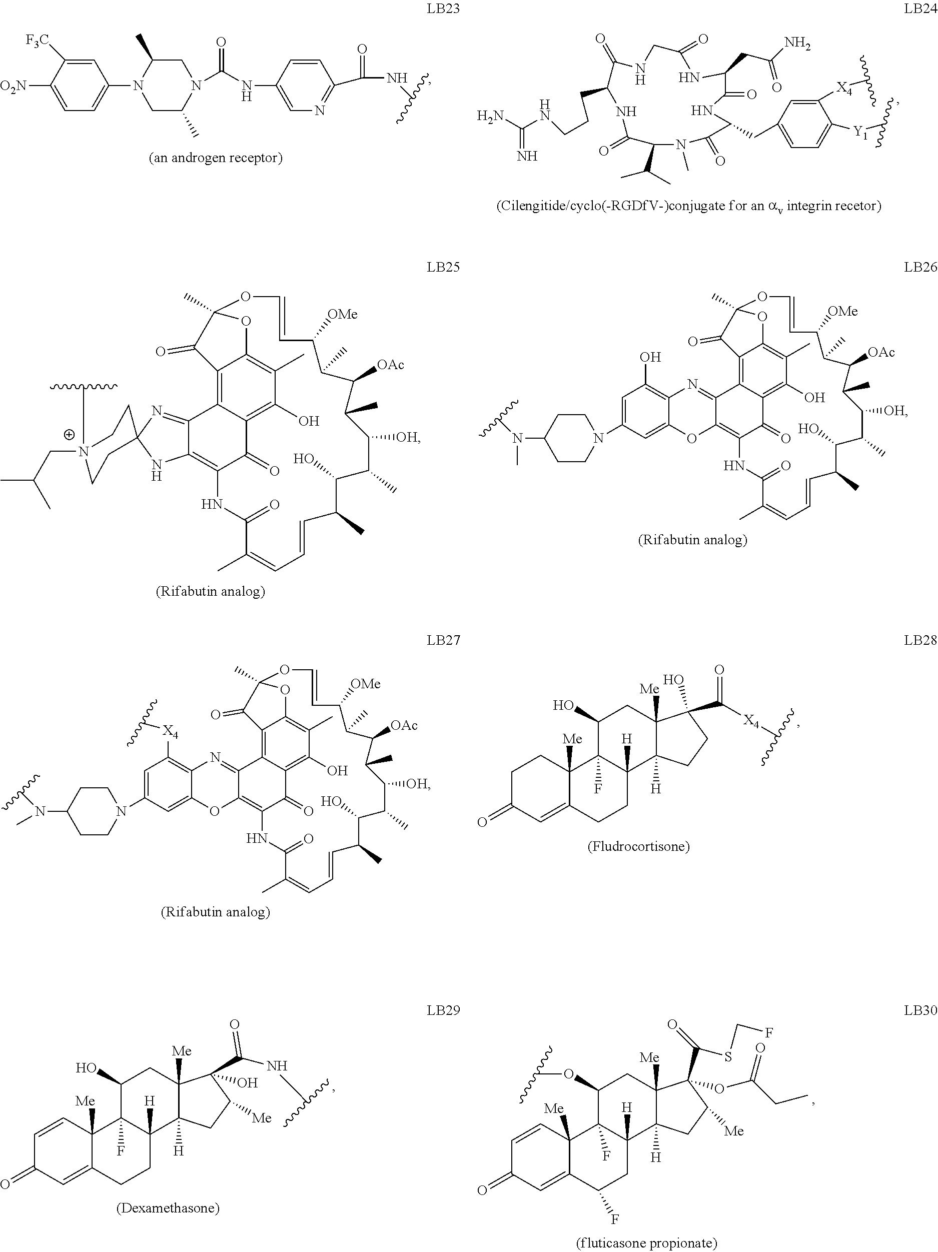 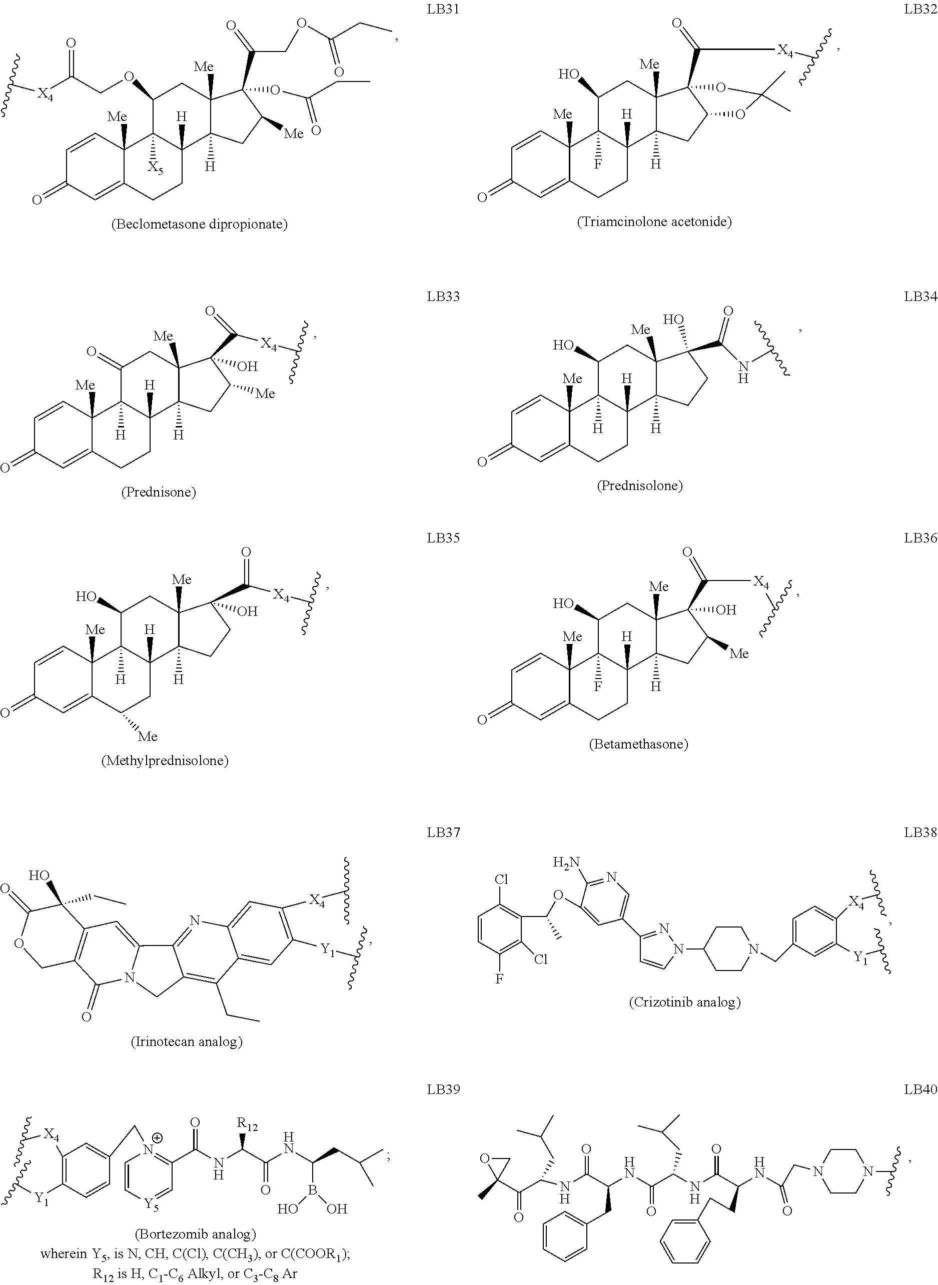 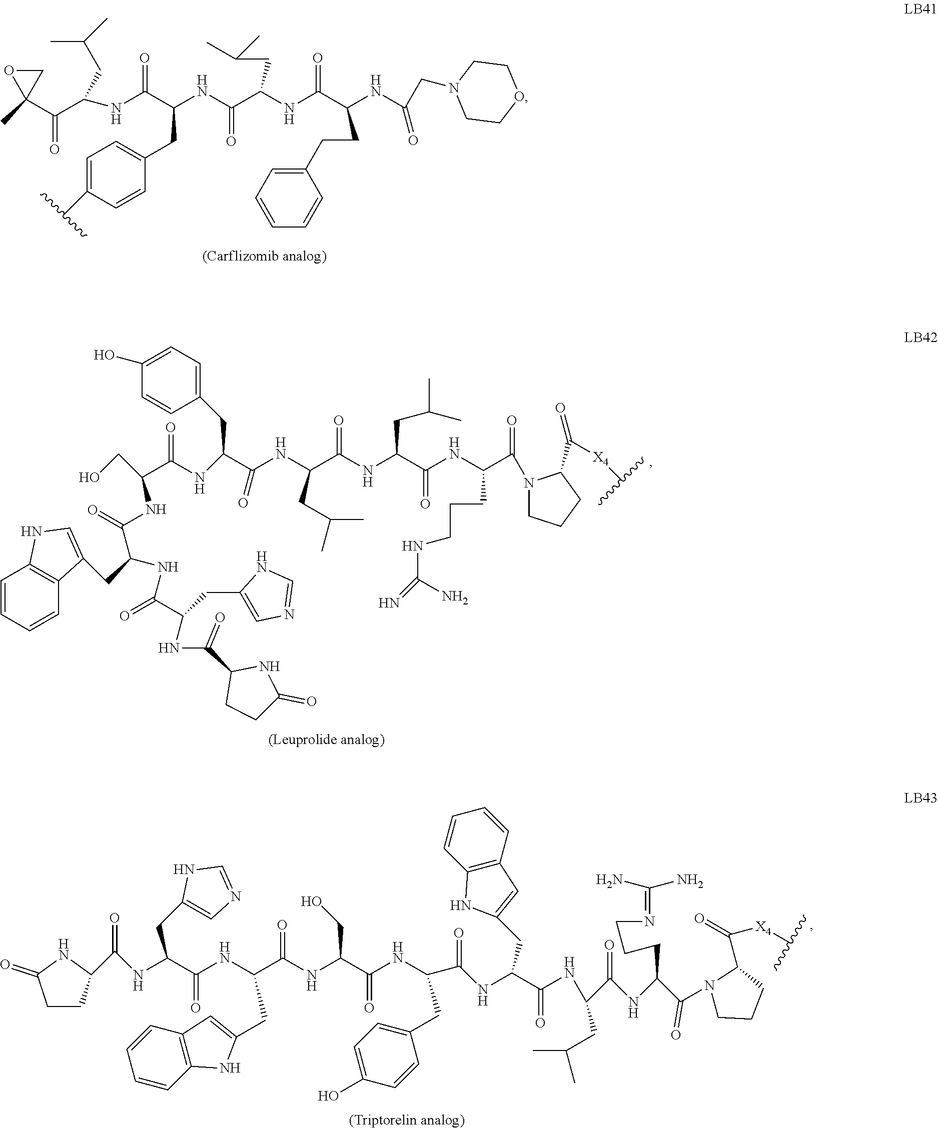 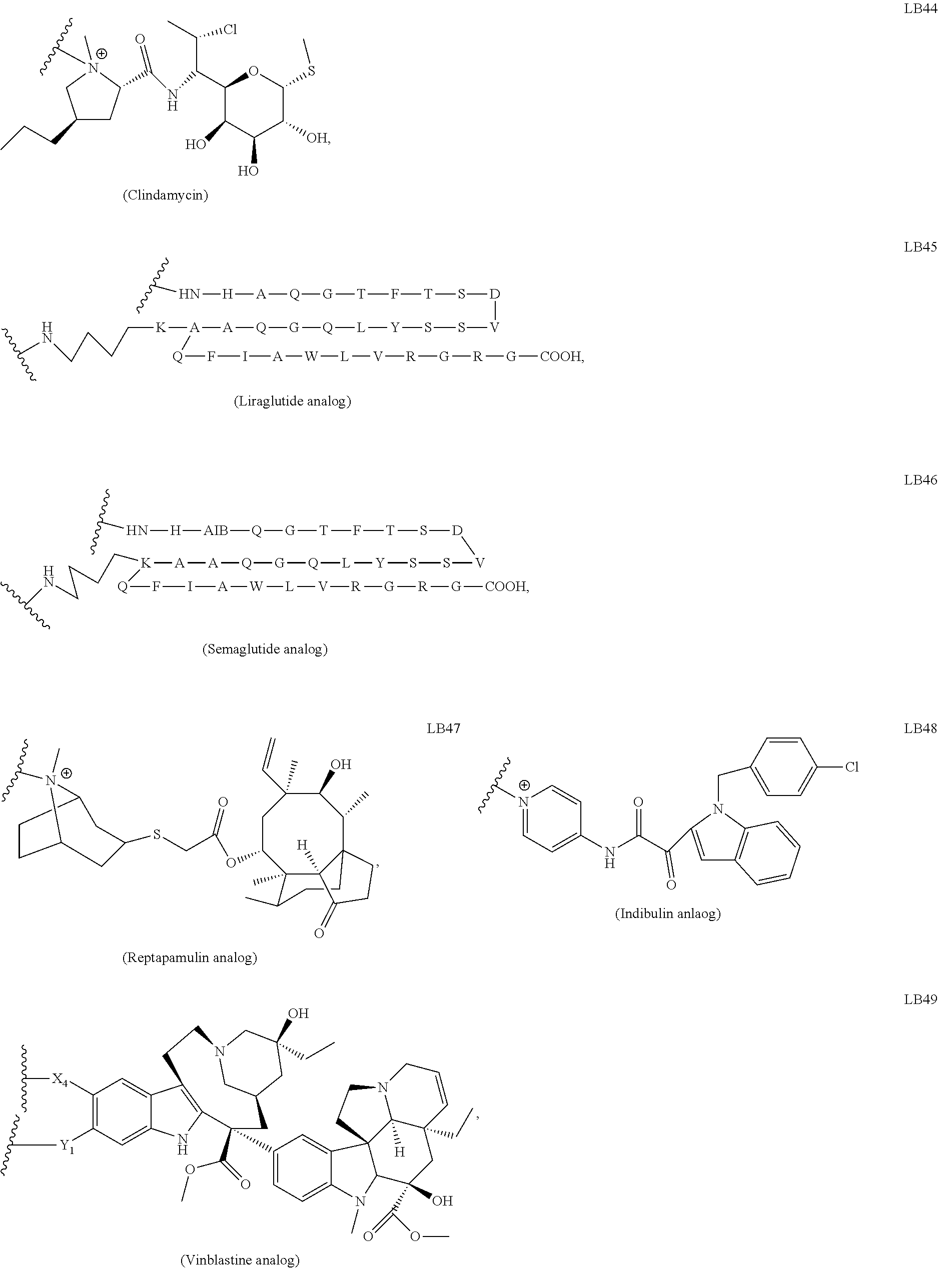 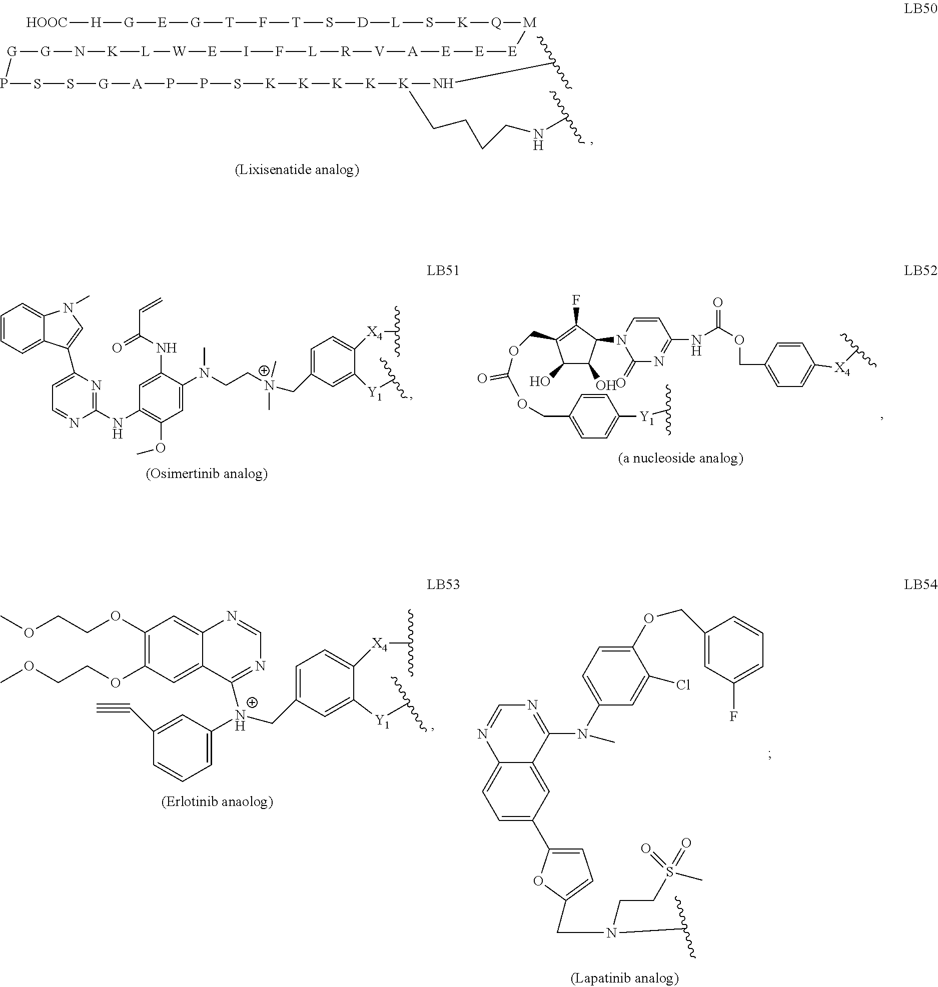
(E): one, two or more DNA, RNA, mRNA, small interfering RNA (siRNA), microRNA (miRNA), and PIWI interacting RNAs (piRNA): 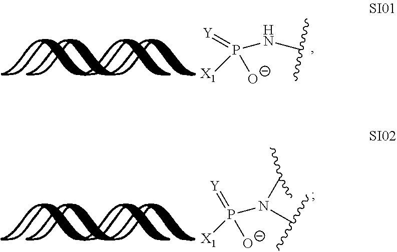
(F): an immunotoxin: Diphtheria toxin (DT), Cholera toxin (CT), Trichosanthin (TCS), Dianthin, Pseudomonas exotoxin A (ETA′), Erythrogenic toxins, Diphtheria toxin, AB toxins, Type III exotoxins;
wherein “ [symbol] ” is a site to link the side chain linker; [symbol] is single or double strands of DNA, RNA, mRNA, siRNA, miRNA, or piRNA; X₄ and Y₁ are independently O, NH, NHNH, NR₁, S, C(O)O, C(O)NH, OC(O)NH, OC(O)O, NHC(O)NH, NHC(O)S, OC(O)N(R₁), N(R₁)C(O)N(R₁), CH₂, C(O)NHNHC(O) or C(O)NR₁; X₁ is H, CH₂, OH, O, C(O), C(O)NH, C(O)N(R₁), R₁, NHR₁, NR₁, C(O)R₁ or C(O)O; X₅ is H, CH₃, F, or Cl; M₁ and M₂ are independently H, Na, K, Ca, Mg, NH₄, or N(R₁₂R_{12′}R₁₃R_{13′}); R₁₂, R_{12′}, R₁₃ and R_{13′} are defined in claim 22.

33. The compound according to claim 22, wherein when the cell-binding agent links to V₁ and/or V₂, of Formula (I), or when the cell-binding agent directly links to L₁ and/or L₂ of Formula (I), wherein V₁ and/or V₂ are absent, the conjugate compound has one or more of the following linkage structures: 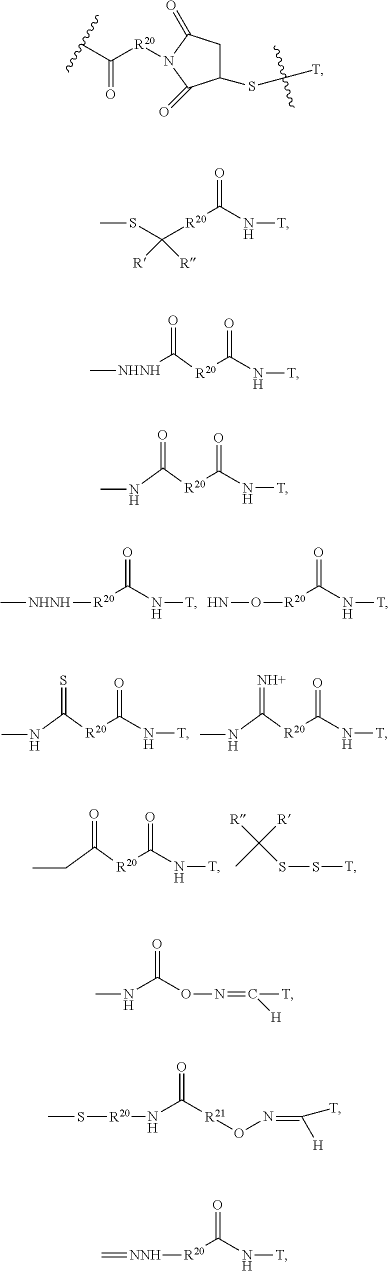 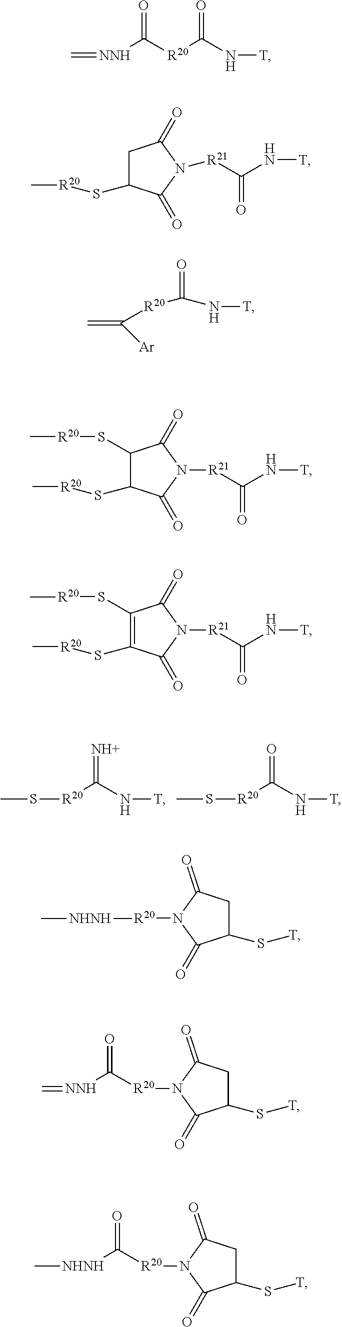 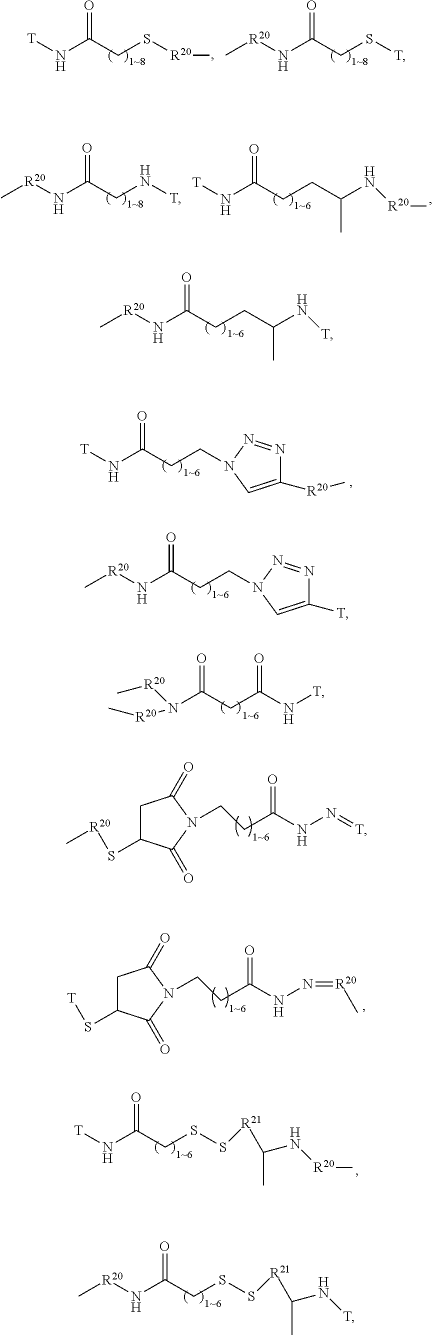 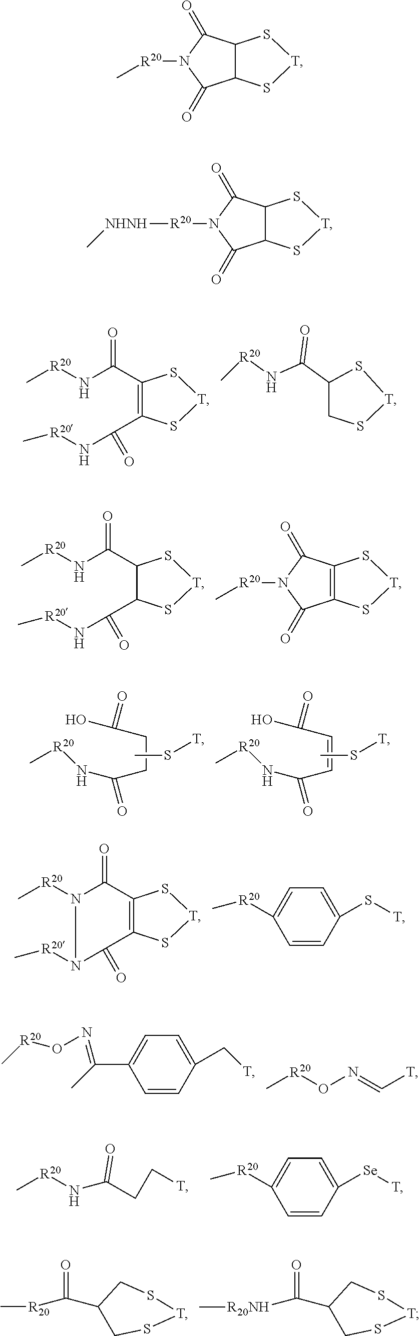 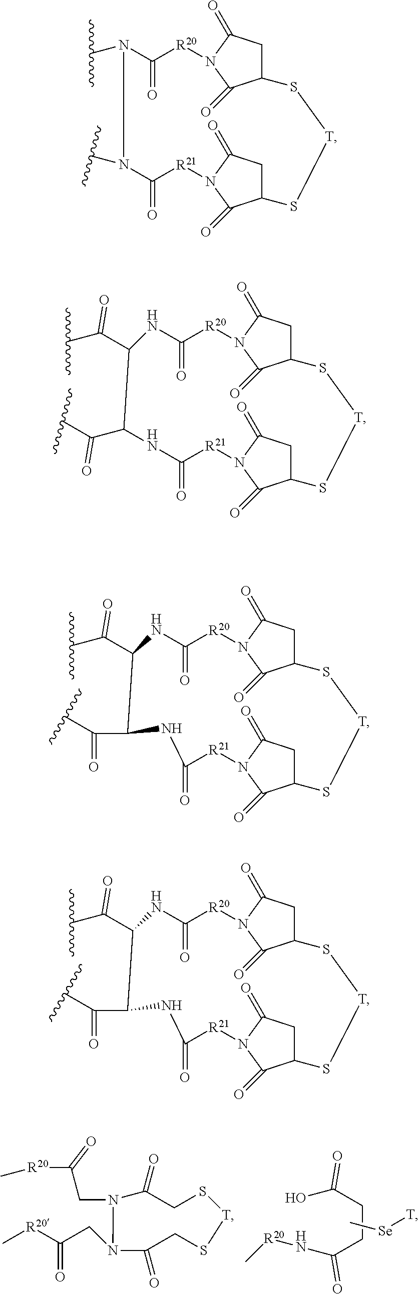 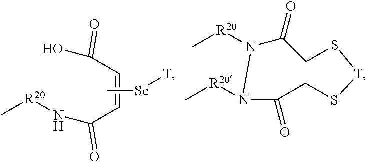
wherein R²⁰ and R²¹ are independently C₁-C₈ alkyl; C₂-C₈ heteroalkyl, or heterocyclic; C₃-C₈ aryl, Ar-alkyl, cycloalkyl, alkylcycloalkyl, heterocycloalkyl, heteroalkylcycloalkyl, carbocyclic, or alkylcarbonyl; or C₂-C₁₀₀ polyethylene glycol having formula of (CH₂CH₂O)ₚ.

34. The compound according to claim 22, wherein the cell binding agent is capable of targeting against a tumor cell, a virus infected cell, a microorganism infected cell, a parasite infected cell, an autoimmune disease cell, an activated tumor cells, a myeloid cell, an activated T-cell, an affecting B cell, or a melanocyte, or cells expressing any one of the following antigens or receptors: CD1, CD1a, CD1b, CD1c, CD1d, CD1e, CD2, CD3, CD3d, CD3e, CD3g, CD4, CD5, CD6, CD7, CD8, CD8a, CD8b, CD9, CD10, CD11a, CD11b, CD11c, CD11d, CD12w, CD14, CD15, CD16, CD16a, CD16b, CDw17, CD18, CD19, CD20, CD21, CD22, CD23, CD24, CD25, CD26, CD27, CD28, CD29, CD30, CD31, CD32, CD32a, CD32b, CD33, CD34, CD35, CD36, CD37, CD38, CD39, CD40, CD41, CD42, CD42a, CD42b, CD42c, CD42d, CD43, CD44, CD45, CD46, CD47, CD48, CD49b, CD49c, CD49c, CD49d, CD49f, CD50, CD51, CD52, CD53, CD54, CD55, CD56, CD57, CD58, CD59, CD60, CD60a, CD60b, CD60c, CD61, CD62E, CD62L, CD62P, CD63, CD64, CD65, CD65s, CD66, CD66a, CD66b, CD66c, CD66d, CD66e, CD66f, CD67, CD68, CD69, CD70, CD71, CD72, CD73, CD74, CD75, CD75s, CD76, CD77, CD78, CD79, CD79a, CD79b, CD80, CD81, CD82, CD83, CD84, CD85, CD85a, CD85b, CD85c, CD85d, CD85e, CD85f, CD85g, CD85g, CD85i, CD85j, CD85k, CD85m, CD86, CD87, CD88, CD89, CD90, CD91, CD92, CD93, CD94, CD95, CD96, CD97, CD98, CD99, CD100, CD101, CD102, CD103, CD104, CD105, CD106, CD107, CD107a, CD107b, CD108, CD109, CD110, CD111, CD112, CD113, CD114, CD115, CD116, CD117, CD118, CD119, CD120, CD120a, CD120b, CD121, CD121a, CD121b, CD122, CD123, CD123a, CD124, CD125, CD126, CD127, CD128, CD129, CD130, CD131, CD132, CD133, CD134, CD135, CD136, CD137, CD138, CD139, CD140, CD140a, CD140b, CD141, CD142, CD143, CD144, CD145, CDw145, CD146, CD147, CD148, CD149, CD150, CD151, CD152, CD153, CD154, CD155, CD156, CD156a, CD156b, CD156c, CD156d, CD157, CD158, CD158a, CD158b1, CD158b2, CD158c, CD158d, CD158e1, CD158e2, CD158f2, CD158g, CD158h, CD158i, CD158j, CD158k, CD159, CD159a, CD159b, CD159c, CD160, CD161, CD162, CD163, CD164, CD165, CD166, CD167, CD167a, CD167b, CD168, CD169, CD170, CD171, CD172, CD172a, CD172b, CD172g, CD173, CD174, CD175, CD175s, CD176, CD177, CD178, CD179, CD179a, CD179b, CD180, CD181, CD182, CD183, CD184, CD185, CD186, CDw186, CD187, CD188, CD189, CD190, CD191, CD192, CD193, CD194, CD195, CD196, CD197, CD198, CD199, CDw198, CDw199, CD200, CD201, CD202, CD202 (a, b), CD203, CD203c, CD204, CD205, CD206, CD207, CD208, CD209, CD210, CDw210a, CDw210b, CD211, CD212, CD213, CD213a1, CD213a2, CD214, CD215, CD216, CD217, CD218, CD218a, CD218, CD219b, CD220, CD221, CD222, CD223, CD224, CD225, CD226, CD227, CD228, CD229, CD230, CD231, CD232, CD233, CD234, CD235, CD235a, CD235b, CD236, CD237, CD238, CD239, CD240, CD240ce, CD240d, CD241, CD242, CD243, CD244, CD245, CD246, CD247, CD248, CD249, CD250, CD251, CD252, CD253, CD254, CD255, CD256, CD257, CD258, CD259, CD260, CD261, CD262, CD263, CD264, CD265, CD266, CD267, CD268, CD269, CD270, CD271, CD272, CD273, CD274, CD275, CD276, CD277, CD278, CD279, CD281, CD282, CD283, CD284, CD285, CD286, CD287, CD288, CD289, CD290, CD291, CD292, CD293, CD294, CD295, CD296, CD297, CD298, CD299, CD300, CD300a, CD300b, CD300c, CD301, CD302, CD303, CD304, CD305, CD306, CD307, CD307a, CD307b, CD307c, CD307d, CD307e, CD307f, CD308, CD309, CD310, CD311, CD312, CD313, CD314, CD315, CD316, CD317, CD318, CD319, CD320, CD321, CD322, CD323, CD324, CD325, CD326, CD327, CD328, CD329, CD330, CD331, CD332, CD333, CD334, CD335, CD336, CD337, CD338, CD339, CD340, CD341, CD342, CD343, CD344, CD345, CD346, CD347, CD348, CD349, CD350, CD351, CD352, CD353, CD354, CD355, CD356, CD357, CD358, CD359, CD360, CD361, CD362, CD363, CD364, CD365, CD366, CD367, CD368, CD369, CD370, CD371, CD372, CD373, CD374, CD375, CD376, CD377, CD378, CD379, CD381, CD382, CD383, CD384, CD385, CD386, CD387, CD388, CD389, CRIPTO, CRIPTO, CR, CR1, CRGF, CRIPTO, CXCR5, LY64, TDGF1, 4-1BB, APO2, ASLG659, BMPR1B, 4-1BB, 5AC, 5T4 (Trophoblastic glycoprotein, TPBG, 5T4, Wnt-Activated Inhibitory Factor 1 or WAIF1), Adenocarcinoma antigen, AGS-5, AGS-22M6, Activin receptor-like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), BCMA, B-lymphoma cell, bcr-abl, Bombesin, BORIS, C5, C242 antigen, CA125 (carbohydrate antigen 125, MUC16), CA-IX (or CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4), CCR5, CD3E (epsilon), CEA (Carcinoembryonic antigen), CEACAM3, CEACAM5 (carcino-embryonic antigen), CFD (Factor D), Ch4D5, Cholecystokinin 2 (CCK2R), CLDN18 (Claudin-18), Clumping factor A, cMet, CRIPTO, FCSF1R (Colony stimulating factor 1 receptor), CSF2 (colony stimulating factor 2, Granulocyte-macrophage colony-stimulating factor (GM-CSF)), CSP4, CTLA4 (cytotoxic T-lymphocyte-associated protein 4), CTAA16.88 tumor antigen, CXCR4, C-X-C chemokine receptor type 4, cyclic ADP ribose hydrolase, Cyclin B1, CYP1B1, Cytomegalovirus, Cytomegalovirus glycoprotein B, Dabigatran, DLL3 (delta-like-ligand 3), DLL4 (delta-like-ligand 4), DPP4 (Dipeptidyl-peptidase 4), DR5 (Death receptor 5), E. coli shiga toxin type-1, E. coli shiga toxin type-2, ED-B, EGFL7 (EGF-like domain-containing protein 7), EGFR, EGFRII, EGFRvIII, Endoglin, Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1F protein of respiratory syncytial virus, Frizzled receptor, Fucosyl GM1, GD2 ganglioside, G-28 (a cell surface antigen glyvolipid), GD3 idiotype, GloboH, Glypican 3, N-glycolylneuraminic acid, GM3, GMCSF receptor α-chain, Growth differentiation factor 8, GP100, GPNMB (Trans-membrane glycoprotein NMB), GUCY2C (Guanylate cyclase 2C, guanylyl cyclase C(GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, interleukins (comprising IL-1, IL-2, IL-3, IL-4, IL-5, IL-6, IL-6R, IL-7, IL-8, IL-9, IL-10, IL-11, IL-12, IL-13, IL-15, IL-17, IL-17A, IL-18, IL-19, IL-20, IL-21, IL-22, IL-23, IL-27, or IL-28), IL31RA, ILGF2 (Insulin-like growth factor 2), Integrins (α4, α_{IIb}β₃, αvβ3, α₄β₇, α5β1, α6β4, α7β7, αllβ3, α5β5, αvβ5), Interferon gamma-induced protein, ITGA2, ITGB2, KIR2D, Kappa Ig, LCK, Le, Legumain, Lewis-Y antigen, LFA-1 (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MART1, MCP-1, MIF (Macrophage migration inhibitory factor, or glycosylation-inhibiting factor (GIF)), MS4A1 (membrane-spanning 4-domains subfamily A member 1), MSLN (mesothelin), MUC1 (Mucin 1, cell surface associated (MUC1) or polymorphic epithelial mucin (PEM)), MUC1-KLH, MUC16 (CA125), MCP1 (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES1, P21, p53 nonmutant, P97, Page4, PAP, Paratope of anti-(N-glycolylneuraminic acid), PAX3, PAX5, PCSK9, PDCD1 (PD-1, Programmed cell death protein 1), PDGF-Rα (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI)), Rhesus factor, RANKL, RhoC, Ras mutant, RGS5, ROBO4, Respiratory syncytial virus, RON, ROR1, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-α, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF-13B (tumor necrosis factor receptor superfamily member 13B), TPBG (trophoblast glycoprotein), TRAIL-R₁ (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2 (Trop2), Tyrosinase, VCAM-1, VEGF, VEGF-A, VEGF-2, VEGFR-1, VEGFR2, or vimentin, WT1, XAGE 1, or cells expressing any insulin growth factor receptors, or any epidermal growth factor receptors.

35. The compound according to claim 34, wherein the tumor cell is selected from the group consisting of lymphoma cells, myeloma cells, renal cells, breast cancer cells, prostate cancer cells, ovarian cancer cells, colorectal cancer cells, gastric cancer cells, squamous cancer cells, small-cell lung cancer cells, none small-cell lung cancer cells, testicular cancer cells, malignant cells, and cells that grow and divide at an unregulated, quickened pace to cause cancers.

36. A pharmaceutical composition comprising a therapeutically effective amount of the conjugate compound of claim 22, or a combination of the conjugate compounds thereof, and a pharmaceutically acceptable salt, carrier, diluent, or excipient therefor, for treatment or prevention of a cancer, or an autoimmune disease, or an infectious disease.

37. The pharmaceutical composition according to claim 36, which is either in a liquid formula or in a formulated lyophilized solid/powder, comprising by weight of: 0.01%-99% of one or more of the conjugate compounds; 0.0%-20.0% of one or more polyols; 0.0%-2.0% of one or more surfactants; 0.0%-5.0% of one or more preservatives; 0.0%-30% of one or more amino acids; 0.0%-5.0% of one or more antioxidants; 0.0%-0.3% of one or more metal chelating agents; 0.0%-30.0% of one or more buffer salts for adjusting pH of the composition to pH 4.5 to 7.5; and 0.0%-30.0% of one or more of isotonic agent for adjusting osmotic pressure between about 250 to 350 mOsm when reconstituted for administration to a patient;
wherein the polyol is selected from the group consisting of fructose, mannose, maltose, lactose, arabinose, xylose, ribose, rhamnose, galactose, glucose, sucrose, trehalose, sorbose, melezitose, raffinose, mannitol, xylitol, erythritol, maltitol, lactitol, erythritol, threitol, sorbitol, glycerol, and L-gluconate and its metallic salts;
wherein the surfactant is selected from the group consisting of polysorbate 20, polysorbate 40, polysorbate 65, polysorbate 80, polysorbate 81, or polysorbate 85, poloxamer, poly(ethylene oxide)-poly(propylene oxide), polyethylene-polypropylene, Triton; sodium dodecyl sulfate (SDS), sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (lauroamidopropyl); myristamidopropyl-, palmidopropyl-, or isostearamidopropyl-dimethylamine; sodium methyl cocoyl-, or disodium methyl oleyl-taurate; dodecyl betaine, dodecyl dimethylamine oxide, cocamidopropyl betaine and coco ampho glycinate; or isostearyl ethylimidonium ethosulfate; polyethyl glycol, polypropyl glycol, and copolymers of ethylene and propylene glycol;
wherein the preservative is benzyl alcohol, octadecyldimethylbenzyl ammonium chloride, hexamethonium chloride, benzalkonium chloride, benzethonium chloride, phenol, butyl and benzyl alcohol, alkyl parabens such as methyl or propyl paraben, catechol, resorcinol, cyclohexanol, 3-pentanol, or m-cresol;
wherein the amino acid is arginine, cystine, glycine, lysine, histidine, ornithine, isoleucine, leucine, alanine, glycine glutamic acid or aspartic acid;
wherein the antioxidant is ascorbic acid, glutathione, cystine or methionine;
wherein the chelating agent is EDTA or EGTA;
wherein the buffer salt is sodium, potassium, ammonium, or trihydroxyethylamino salts of citric acid, ascorbic acid, gluconic acid, carbonic acid, tartaric acid, succinic acid, acetic acid or phthalic acid; Tris or tromethamine hydrochloride, phosphate or sulfate; arginine, glycine, glycylglycine, or histidine with anionic acetate, chloride, phosphate, sulfate, or succinate salts;
wherein the tonicity agent is mannitol, sorbitol, sodium acetate, potassium chloride, sodium phosphate, potassium phosphate, trisodium citrate, or sodium chloride.

38. The pharmaceutical composition according to claim 36, which is packed in a vial, bottle, pre-filled syringe, or pre-filled auto-injector syringe, in a form of a liquid or lyophilized solid.

39. The compound of claim 22, having in vitro, in vivo or ex vivo cell killing activity.

40. A method for synergistical treatment or prevention of a cancer, or an autoimmune disease, or an infectious disease, comprising administering to a subject in need thereof the pharmaceutical composition according to claim 36, concurrently with a synergistic agent comprising a chemotherapeutic agent, a radiation therapy agent, an immunotherapy agent, an autoimmune disorder agent, an anti-infectious agent or another conjugate.

41. The method according to claim 40, wherein the chemotherapeutic agent is one or more selected from the group consisting of:
(1) a) an alkylating agent selected from nitrogen mustards: chlorambucil, chlornaphazine, cyclophosphamide, dacarbazine, estramustine, ifosfamide, mechlorethamine, mechlorethamine oxide hydrochloride, mannomustine, mitobronitol, melphalan, mitolactol, pipobroman, novembichin, phenesterine, prednimustine, thiotepa, trofosfamide, uracil mustard; CC-1065 and adozelesin, carzelesin, bizelesin or their synthetic analogues; duocarmycin and its synthetic analogues, KW-2189, CBI-TMI, or CBI dimers; benzodiazepine dimers or pyrrolobenzodiazepine (PBD) dimers, tomaymycin dimers, indolinobenzodiazepine dimers, imidazobenzothiadiazepine dimers, or oxazolidinobenzodiazepine dimers; Nitrosoureas: comprising carmustine, lomustine, chlorozotocin, fotemustine, nimustine, ranimustine; Alkylsulphonates: including busulfan, treosulfan, improsulfan and piposulfan); Triazenes or dacarbazine; Platinum containing compounds: comprising carboplatin, cisplatin, and oxaliplatin; aziridines, benzodopa, carboquone, meturedopa, or uredopa; ethylenimines and methylamelamines including altretamine, triethylenemelamine, trietylenephosphoramide, triethylenethiophosphoramide and trimethylolomelamine;
b) a plant alkaloid selected from the group consisting of Vinca alkaloids: including vincristine, vinblastine, vindesine, vinorelbine, and navelbin; Taxoids: comprising paclitaxel, docetaxol and their analogs, Maytansinoids including DM1, DM2, DM3, DM4, DM5, DM6, DM7, maytansine, ansamitocins and their analogs, cryptophycins (including the group of cryptophycin 1 and cryptophycin 8); epothilones, eleutherobin, discodermolide, bryostatins, dolostatins, auristatins, tubulysins, cephalostatins; pancratistatin; a sarcodictyin; spongistatin;
c) a DNA Topoisomerase Inhibitor: selected from the groups of Epipodophyllins: comprising 9-aminocamptothecin, camptothecin, crisnatol, daunomycin, etoposide, etoposide phosphate, irinotecan, mitoxantrone, novantrone, retinoic acids (or retinols), teniposide, topotecan, 9-nitrocamptothecin or RFS 2000; and mitomycins and their analogs;
d) an antimetabolite selected from the group consisting of {[Anti-folate: (DHFR inhibitors: comprising methotrexate, trimetrexate, denopterin, pteropterin, aminopterin (4-aminopteroic acid) or folic acid analogues); IMP dehydrogenase Inhibitors: (including mycophenolic acid, tiazofurin, ribavirin, EICAR); Ribonucleotide reductase Inhibitors: (including hydroxyurea, deferoxamine)]; [Pyrimidine analogs: Uracil analogs: (including ancitabine, azacitidine, 6-azauridine, capecitabine, carmofur, cytarabine, dideoxyuridine, doxifluridine, enocitabine, 5-Fluorouracil, floxuridine, ratitrexed); Cytosine analogs: (including cytarabine, cytosine arabinoside, fludarabine); Purine analogs: (including azathioprine, fludarabine, mercaptopurine, thiamiprine, thioguanine)]; folic acid replenisher, frolinic acid};
e) a hormonal therapy selected from Receptor antagonists: [Anti-estrogen: (including megestrol, raloxifene, tamoxifen); LHRH agonists: (including goscrclin, leuprolide acetate); Anti-androgens: (including bicalutamide, flutamide, calusterone, dromostanolone propionate, epitiostanol, goserelin, leuprolide, mepitiostane, nilutamide, testolactone, trilostane and other androgens inhibitors)]; Retinoids/Deltoids: [Vitamin D3 analogs: (including CB 1093, EB 1089 KH 1060, cholecalciferol, ergocalciferol); Photodynamic therapies: (including verteporfin, phthalocyanine, photosensitizer Pc4, demethoxyhypocrellin A); Cytokines: (comprising Interferon-alpha, Interferon-gamma, tumor necrosis factor (TNFs), human proteins containing a TNF domain)]};
f) a kinase inhibitor selected from the group consisting of BIBW 2992 (anti-EGFR/Erb2), imatinib, gefitinib, pegaptanib, sorafenib, dasatinib, sunitinib, erlotinib, nilotinib, lapatinib, axitinib, pazopanib, vandetanib, E7080 (anti-VEGFR2), mubritinib, ponatinib, bafetinib, bosutinib, cabozantinib, vismodegib, iniparib, ruxolitinib, CYT387, axitinib, tivozanib, sorafenib, bevacizumab, cetuximab, Trastuzumab, Ranibizumab, Panitumumab, and ispinesib;
g) a poly (ADP-ribose) polymerase (PARP) inhibitor selected from the group of olaparib, niraparib, iniparib, talazoparib, veliparib, CEP 9722, E7016, BGB-290, and 3-aminobenzamide;
h) an antibiotic selected from the group consisting of an enediyne antibiotic (selected from the group of calicheamicin, calicheamicin γ1, δ1, β1 or β1; dynemicin, including dynemicin A and deoxydynemicin; esperamicin, kedarcidin, C-1027, maduropeptin, or neocarzinostatin chromophore and related chromoprotein enediyne antibiotic chromomophores), aclacinomycins, actinomycin, authramycin, azaserine, bleomycins, cactinomycin, carabicin, carminomycin, carzinophilin; chromomycins, dactinomycin, daunorubicin, detorubicin, 6-diazo-5-oxo-L-norleucine, doxorubicin, morpholino-doxorubicin, cyanomorpholino-doxorubicin, 2-pyrrolino-doxorubicin and deoxydoxorubicin, epirubicin, eribulin, esorubicin, idarubicin, marcellomycin, nitomycins, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin;
i) a polyketide (acetogenin), bullatacin and bullatacinone; gemcitabine, epoxomicins andcarfilzomib, bortezomib, thalidomide, lenalidomide, pomalidomide, tosedostat, zybrestat, PLX4032, STA-9090, Stimuvax, allovectin-7, Xegeva, Provenge, Yervoy, Isoprenylation inhibitors and Lovastatin, Dopaminergic neurotoxins and 1-methyl-4-phenylpyridinium ion, Cell cycle inhibitors (including staurosporine), Actinomycins (including Actinomycin D, dactinomycin), amanitins, Bleomycins (including bleomycin A2, bleomycin B2, peplomycin), Anthracyclines (including daunorubicin, doxorubicin (adriamycin), idarubicin, epirubicin, pirarubicin, zorubicin, mtoxantrone, MDR inhibitors or verapamil, Ca²⁺ ATPase inhibitors or thapsigargin, Histone deacetylase inhibitors ((including Vorinostat, Romidepsin, Panobinostat, Valproic acid, Mocetinostat (MGCD0103), Belinostat, PCI-24781, Entinostat, SB939, Resminostat, Givinostat, AR-42, CUDC-101, sulforaphane, Trichostatin A); Thapsigargin, Celecoxib, glitazones, epigallocatechin gallate, Disulfiram, Salinosporamide A; Anti-adrenals, selected from the group of aminoglutethimide, mitotane, trilostane; aceglatone; aldophosphamide glycoside; aminolevulinic acid; amsacrine; arabinoside, bestrabucil; bisantrene; edatraxate; defofamine; demecolcine; diaziquone; eflornithine (DFMO), elfomithine; elliptinium acetate, etoglucid; gallium nitrate; gacytosine, hydroxyurea; ibandronate, lentinan; lonidamine; mitoguazone; mitoxantrone; mopidamol; nitracrine; pentostatin; phenamet; pirarubicin; podophyllinic acid; 2-ethylhydrazide; procarbazine; PSK©; razoxane; rhizoxin; sizofiran; spirogermanium; tenuazonic acid; triaziquone; 2, 2′,2″-trichlorotriethylamine; trichothecenes (including the group of T-2 toxin, verrucarin A, roridin A and anguidine); urethane, siRNA, antisense drugs;
(2) an anti-autoimmune disease agent: cyclosporine, cyclosporine A, aminocaproic acid, azathioprine, bromocriptine, chlorambucil, chloroquine, cyclophosphamide, corticosteroids (including the group consisting of amcinonide, betamethasone, budesonide, hydrocortisone, flunisolide, fluticasone propionate, fluocortolone danazol, dexamethasone, Triamcinolone acetonide, beclometasone dipropionate), DHEA, enanercept, hydroxychloroquine, infliximab, meloxicam, methotrexate, mofetil, mycophenylate, prednisone, sirolimus, tacrolimus;
(3) an anti-infectious disease agents comprising:
a) aminoglycosides: amikacin, astromicin, gentamicin (netilmicin, sisomicin, isepamicin), hygromycin B, kanamycin (amikacin, arbekacin, bekanamycin, dibekacin, tobramycin), neomycin (framycetin, paromomycin, ribostamycin), netilmicin, spectinomycin, streptomycin, tobramycin, verdamicin;
b) amphenicols: azidamfenicol, chloramphenicol, florfenicol, thiamphenicol;
c) ansamycins: geldanamycin, herbimycin;
d) Carbapenems: biapenem, doripenem, ertapenem, imipenem, cilastatin, meropenem, panipenem;
e) Cephems: carbacephem (loracarbef), cefacetrile, cefaclor, cefradine, cefadroxil, cefalonium, cefaloridine, cefalotin or cefalothin, cefalexin, cefaloglycin, cefamandole, cefapirin, cefatrizine, cefazaflur, cefazedone, cefazolin, cefbuperazone, cefcapene, cefdaloxime, cefepime, cefminox, cefoxitin, cefprozil, cefroxadine, ceftezole, cefuroxime, cefixime, cefdinir, cefditoren, cefepime, cefetamet, cefmenoxime, cefodizime, cefonicid, cefoperazone, ceforanide, cefotaxime, cefotiam, cefozopran, cephalexin, cefpimizole, cefpiramide, cefpirome, cefpodoxime, cefprozil, cefquinome, cefsulodin, ceftazidime, cefteram, ceftibuten, ceftiolene, ceftizoxime, ceftobiprole, ceftriaxone, cefuroxime, cefuzonam, cephamycin (including cefoxitin, cefotetan, cefmetazole), oxacephem (flomoxef, latamoxef);
f) Glycopeptides: bleomycin, vancomycin (including oritavancin, telavancin), teicoplanin (dalbavancin), ramoplanin;
g) Glycylcyclines: tigecycline;
h) β-Lactamase inhibitors: penam (sulbactam, tazobactam), clavam (clavulanic acid);
i) Lincosamides: clindamycin, lincomycin;
j) Lipopeptides: daptomycin, A54145, calcium-dependent antibiotics (CDA);
k) Macrolides: azithromycin, cethromycin, clarithromycin, dirithromycin, erythromycin, flurithromycin, josamycin, ketolide (telithromycin, cethromycin), midecamycin, miocamycin, oleandomycin, rifamycins (rifampicin, rifampin, rifabutin, rifapentine), rokitamycin, roxithromycin, spectinomycin, spiramycin, tacrolimus (FK506), troleandomycin, telithromycin;
l) Monobactams: aztreonam, tigemonam;
m) Oxazolidinones: linezolid;
n) Penicillins: amoxicillin, ampicillin, pivampicillin, hetacillin, bacampicillin, metampicillin, talampicillin, azidocillin, azlocillin, benzylpenicillin, benzathine benzylpenicillin, benzathine phenoxymethylpenicillin, clometocillin, procaine benzylpenicillin, carbenicillin (carindacillin), cloxacillin, dicloxacillin, epicillin, flucloxacillin, mecillinam (pivmecillinam), mezlocillin, meticillin, nafcillin, oxacillin, penamecillin, penicillin, pheneticillin, phenoxymethylpenicillin, piperacillin, propicillin, sulbenicillin, temocillin, ticarcillin;
o) Polypeptides: bacitracin, colistin, polymyxin B;
p) Quinolones: alatrofloxacin, balofloxacin, ciprofloxacin, clinafloxacin, danofloxacin, difloxacin, enoxacin, enrofloxacin, floxin, garenoxacin, gatifloxacin, gemifloxacin, grepafloxacin, kano trovafloxacin, levofloxacin, lomefloxacin, marbofloxacin, moxifloxacin, nadifloxacin, norfloxacin, orbifloxacin, ofloxacin, pefloxacin, trovafloxacin, grepafloxacin, sitafloxacin, sparfloxacin, temafloxacin, tosufloxacin, trovafloxacin;
q) Streptogramins: pristinamycin, quinupristin/dalfopristin;
r) Sulfonamides: mafenide, prontosil, sulfacetamide, sulfamethizole, sulfanilimide, sulfasalazine, sulfisoxazole, trimethoprim, trimethoprim-sulfamethoxazole (co-trimoxazole);
s) Steroid antibacterials: selected from fusidic acid;
t) Tetracyclines: doxycycline, chlortetracycline, clomocycline, demeclocycline, lymecycline, meclocycline, metacycline, minocycline, oxytetracycline, penimepicycline, rolitetracycline, tetracycline, glycylcyclines (including tigecycline);
u) antibiotics: annonacin, arsphenamine, bactoprenol inhibitors (Bacitracin), DADAL/AR inhibitors (cycloserine), dictyostatin, discodermolide, eleutherobin, epothilone, ethambutol, etoposide, faropenem, fusidic acid, furazolidone, isoniazid, laulimalide, metronidazole, mupirocin, mycolactone, NAM synthesis inhibitors (fosfomycin), nitrofurantoin, paclitaxel, platensimycin, pyrazinamide, quinupristin/dalfopristin, rifampicin (rifampin), tazobactam tinidazole, uvaricin;
(4) anti-viral drugs comprising:
a) Entry/fusion inhibitors: aplaviroc, maraviroc, vicriviroc, gp41 (enfuvirtide), PRO 140, CD4 (ibalizumab);
b) Integrase inhibitors: raltegravir, elvitegravir, globoidnan A;
c) Maturation inhibitors: bevirimat, vivecon;
d) Neuraminidase inhibitors: oseltamivir, zanamivir, peramivir;
e) Nucleosides & nucleotides: abacavir, aciclovir, adefovir, amdoxovir, apricitabine, brivudine, cidofovir, clevudine, dexelvucitabine, didanosine (ddI), elvucitabine, emtricitabine (FTC), entecavir, famciclovir, fluorouracil (5-FU), 3′-fluoro-substituted 2′, 3′-dideoxynucleoside analogues (including the group consisting of 3′-fluoro-2′,3′-dideoxythymidine (FLT) and 3′-fluoro-2′,3′-dideoxyguanosine (FLG), fomivirsen, ganciclovir, idoxuridine, lamivudine (3TC), 1-nucleosides (including the group consisting of β-1-thymidine and β-1-2′-deoxycytidine), penciclovir, racivir, ribavirin, stampidine, stavudine (d4T), taribavirin (viramidine), telbivudine, tenofovir, trifluridine valaciclovir, valganciclovir, zalcitabine (ddC), zidovudine (AZT);
f) Non-nucleosides: amantadine, ateviridine, capravirine, diarylpyrimidines (etravirine, rilpivirine), delavirdine, docosanol, emivirine, efavirenz, foscarnet (phosphonoformic acid), imiquimod, interferon alfa, loviride, lodenosine, methisazone, nevirapine, NOV-205, peginterferon alfa, podophyllotoxin, rifampicin, rimantadine, resiquimod (R-848), tromantadine;
g) Protease inhibitors: amprenavir, atazanavir, boceprevir, darunavir, fosamprenavir, indinavir, lopinavir, nelfinavir, pleconaril, ritonavir, saquinavir, telaprevir (VX-950), tipranavir;
h) anti-virus drugs: abzyme, arbidol, calanolide a, ceragenin, cyanovirin-n, diarylpyrimidines, epigallocatechin gallate (EGCG), foscarnet, griffithsin, taribavirin (viramidine), hydroxyurea, KP-1461, miltefosine, pleconaril, portmanteau inhibitors, ribavirin, seliciclib;
(5) a pharmaceutically acceptable salt, acid, derivative, hydrate or hydrated salt; or a crystalline structure; or an optical isomer, racemate, diastereomer or enantiomer of any of the above drugs.

42. The pharmaceutical composition according to claim 40, wherein the synergistic agent is one or more selected from the following drugs: Abatacept, abemaciclib, Abiraterone acetate, Abraxane, Acetaminophen/hydrocodone, Acalabrutinib, aducanumab, Adalimumab, ADXS31-142, ADXS-HER2, afatinib dimaleate, aldesleukin, alectinib, alemtuzumab, Alitretinoin, ado-trastuzumab emtansine, Amphetamine/dextroamphetamine, anastrozole, Aripiprazole, anthracyclines, Aripiprazole, Atazanavir, Atezolizumab, Atorvastatin, Avelumab, Axicabtagene ciloleucel, axitinib, belinostat, BCG Live, Bevacizumab, bexarotene, blinatumomab, Bortezomib, bosutinib, brentuximab vedotin, brigatinib, Budesonide, Budesonide/formoterol, Buprenorphine, Cabazitaxel, Cabozantinib, capmatinib, Capecitabine, carfilzomib, chimeric antigen receptor-engineered T (CAR-T) cells, Celecoxib, ceritinib, Cetuximab, Chidamide, Ciclosporin, Cinacalcet, crizotinib, Cobimetinib, Cosentyx, crizotinib, CTL019, Dabigatran, dabrafenib, dacarbazine, daclizumab, dacomotinib, daptomycin, Daratumumab, Darbepoetin alfa, Darunavir, dasatinib, denileukin diftitox, Denosumab, Depakote, Dexlansoprazole, Dexmethylphenidate, Dexamethasone, DigniCap Cooling System, Dinutuximab, Doxycycline, Duloxetine, Duvelisib, durvalumab, elotuzumab, Emtricibine/Rilpivirine/Tenofovir, disoproxil fumarate, Emtricitbine/tenofovir/efavirenz, Enoxaparin, ensartinib, Enzalutamide, Epoetin alfa, erlotinib, Esomeprazole, Eszopiclone, Etanercept, Everolimus, exemestane, everolimus, exenatide ER, Ezetimibe, Ezetimibe/simvastatin, Fenofibrate, Filgrastim, fingolimod, Fluticasone propionate, Fluticasone/salmeterol, fulvestrant, gazyva, gefitinib, Glatiramer, Goserelin acetate, Icotinib, Imatinib, Ibritumomab tiuxetan, ibrutinib, idelalisib, ifosfamide, Infliximab, imiquimod, ImmuCyst, Immuno BCG, iniparib, Insulin aspart, Insulin detemir, Insulin glargine, Insulin lispro, Interferon alfa, Interferon alfa-1b, Interferon alfa-2a, Interferon alfa-2b, Interferon beta, Interferon beta 1a, Interferon beta 1b, Interferon gamma-1a, lapatinib, Ipilimumab, Ipratropium bromide/salbutamol, Ixazomib, Kanuma, Lanreotide acetate, lenalidomide, lenaliomide, lenvatinib mesylate, letrozole, Levothyroxine, Levothyroxine, Lidocaine, Linezolid, Liraglutide, Lisdexamfetamine, LN-144, lorlatinib, Memantine, Methylphenidate, Metoprolol, Mekinist, mericitabine/Rilpivirine/Tenofovir, Modafinil, Mometasone, Mycidac-C, Necitumumab, neratinib, Nilotinib, niraparib, Nivolumab, ofatumumab, obinutuzumab, olaparib, Olmesartan, Olmesartan/hydrochlorothiazide, Omalizumab, Omega-3 fatty acid ethyl esters, Oncorine, Oseltamivir, Osimertinib, Oxycodone, palbociclib, Palivizumab, panitumumab, panobinostat, pazopanib, pembrolizumab, PD-1 antibody, PD-L1 antibody, Pemetrexed, pertuzumab, Pneumococcal conjugate vaccine, pomalidomide, Pregabalin, ProscaVax, Propranolol, Quetiapine, Rabeprazole, radium 223 chloride, Raloxifene, Raltegravir, ramucirumab, Ranibizumab, regorafenib, ribociclib, Rituximab, Rivaroxaban, romidepsin, Rosuvastatin, ruxolitinib phosphate, Salbutamol, savolitinib, semaglutide, Sevelamer, Sildenafil, siltuximab, Sipuleucel-T, Sitagliptin, Sitagliptin/metformin, Solifenacin, solanezumab, Sonidegib, Sorafenib, Sunitinib, tacrolimus, tacrimus, Tadalafil, tamoxifen, Tafinlar, Talimogene laherparepvec, talazoparib, Telaprevir, talazoparib, Temozolomide, temsirolimus, Tenofovir/emtricitabine, tenofovir disoproxil fumarate, Testosterone gel, Thalidomide, TICE BCG, Tiotropium bromide, Tisagenlecleucel, toremifene, trametinib, Trastuzumab, Trabectedin (ecteinascidin 743), trametinib, tremelimumab, Trifluridine/tipiracil, Tretinoin, Uro-BCG, Ustekinumab, Valsartan, veliparib, vandetanib, vemurafenib, venetoclax, vorinostat, ziv-aflibercept, Zostavax, and their analogs, derivatives, pharmaceutically acceptable salts.
